SEQUENCE LISTING (34 sequences)

SEQ ID NO: 1 - protein, 75 aa, Shigella sonnei
METTVFLSNRSQAVRLPKAVALPENVKRVEVIAVGRTRIITPAGETWDEWFDGHSVSADFMDNREQPGMQERESF

SEQ ID NO: 2 - protein, 75 aa, Shigella flexneri
METTVFLSNRSQAVRLPKAVALPENVKRVEVIAVGRTRIITPAGETWDEWFDGHSVSTDFMDNREQPGMQERESF

SEQ ID NO: 3 - protein, 75 aa, Artificial Sequence
  VapB
METTVFLSNRSLAVRLPKAVALPENVKRVEVIAVGRTRIITPAGETWDEWFDGHSVSADFMDNREQPGMQERESF

SEQ ID NO: 4 - protein, 75 aa, Artificial Sequence
  VapB
METTVFLSNRSAAVRLPKAVALPENVKRVEVIAVGRTRIITPAGETWDEWFDGHSVSADFMDNREQPGMQERESF

SEQ ID NO: 5 - protein, 75 aa, Artificial Sequence
  VapB
METTVALSNRSQAVRLPKAVALPENVKRVEVIAVGRTRIITPAGETWDEWFDGHSVSADFMDNREQPGMQERESF

SEQ ID NO: 6 - protein, 75 aa, Artificial Sequence
  VapB
METTVFLSNRSQAVRLPKAVALPENVKRVEVIAVGRTRIITPAGETWDEWFDGHSVSADAMDNREQPGMQERESF

SEQ ID NO: 7 - protein, 75 aa, Artificial Sequence
  VapB
METTVFLSNRSQAVRLPKAVALPENVKRVEVIAVGRTRIITPAGETWDEWADGHSVSADFMDNREQPGMQERESF

SEQ ID NO: 8 - protein, 75 aa, Artificial Sequence
  VapB
METTVALSNRSAAVRLPKAVALPENVKRVEVIAVGRTRIITPAGETWDEWFDGHSVSADFMDNREQPGMQERESF

SEQ ID NO: 9 - protein, 75 aa, Artificial Sequence
  VapB
METTVFLSNRSAAVRLPKAVALPENVKRVEVIAVGRTRIITPAGETWDEWADGHSVSADFMDNREQPGMQERESF

SEQ ID NO: 10 - protein, 75 aa, Artificial Sequence
  VapB
METTVFLSNRSAAVRLPKAVALPENVKRVEVIAVGRTRIITPAGETWDEWFDGHSVSADAMDNREQPGMQERESF

SEQ ID NO: 11 - protein, 75 aa, Artificial Sequence
  VapB
METTVALSNRSLAVRLPKAVALPENVKRVEVIAVGRTRIITPAGETWDEWFDGHSVSADFMDNREQPGMQERESF

SEQ ID NO: 12 - protein, 75 aa, Artificial Sequence
  VapB
METTVFLSNRSLAVRLPKAVALPENVKRVEVIAVGRTRIITPAGETWDEWADGHSVSADFMDNREQPGMQERESF

SEQ ID NO: 13 - protein, 75 aa, Artificial Sequence
  VapB
METTVFLSNRSLAVRLPKAVALPENVKRVEVIAVGRTRIITPAGETWDEWFDGHSVSADAMDNREQPGMQERESF

SEQ ID NO: 14 - protein, 75 aa, Artificial Sequence
  VapB
METTVFLSNRSLAVRLPKAVALPENVKRVEVIAVGRTRIITPAGETWDEWFDGHSVSTDFMDNREQPGMQERESF

SEQ ID NO: 15 - protein, 75 aa, Artificial Sequence
  VapB
METTVFLSNRSAAVRLPKAVALPENVKRVEVIAVGRTRIITPAGETWDEWFDGHSVSTDFMDNREQPGMQERESF

SEQ ID NO: 16 - protein, 75 aa, Artificial Sequence
  VapB
METTVALSNRSQAVRLPKAVALPENVKRVEVIAVGRTRIITPAGETWDEWFDGHSVSTDFMDNREQPGMQERESF

SEQ ID NO: 17 - protein, 75 aa, Artificial Sequence
  VapB
METTVFLSNRSQAVRLPKAVALPENVKRVEVIAVGRTRIITPAGETWDEWFDGHSVSTDAMDNREQPGMQERESF

SEQ ID NO: 18 - protein, 75 aa, Artificial Sequence; identical to SEQ ID NO: 7
  VapB

SEQ ID NO: 19 - protein, 75 aa, Artificial Sequence
  VapB
METTVALSNRSAAVRLPKAVALPENVKRVEVIAVGRTRIITPAGETWDEWFDGHSVSTDFMDNREQPGMQERESF

SEQ ID NO: 20 - protein, 75 aa, Artificial Sequence
  VapB
METTVFLSNRSAAVRLPKAVALPENVKRVEVIAVGRTRIITPAGETWDEWADGHSVSTDFMDNREQPGMQERESF

SEQ ID NO: 21 - protein, 75 aa, Artificial Sequence
  VapB
METTVFLSNRSAAVRLPKAVALPENVKRVEVIAVGRTRIITPAGETWDEWFDGHSVSTDAMDNREQPGMQERESF

SEQ ID NO: 22 - protein, 75 aa, Artificial Sequence
  VapB
METTVALSNRSLAVRLPKAVALPENVKRVEVIAVGRTRIITPAGETWDEWFDGHSVSTDFMDNREQPGMQERESF

SEQ ID NO: 23 - protein, 75 aa, Artificial Sequence
  VapB
METTVFLSNRSLAVRLPKAVALPENVKRVEVIAVGRTRIITPAGETWDEWADGHSVSTDFMDNREQPGMQERESF

SEQ ID NO: 24 - protein, 75 aa, Artificial Sequence
  VapB
METTVFLSNRSLAVRLPKAVALPENVKRVEVIAVGRTRIITPAGETWDEWFDGHSVSTDAMDNREQPGMQERESF

SEQ ID NO: 25 - protein, 17 aa, Artificial Sequence
  artificial
FMAEREDVVSDEGRFEP

SEQ ID NO: 26 - protein, 17 aa, Artificial Sequence
  artificial
FMAEREDIVSDEGRFDL

SEQ ID NO: 27 - protein, 17 aa, Artificial Sequence
  artificial
FMAEREDIVSDEGRFDL

SEQ ID NO: 28 - protein, 16 aa, Artificial Sequence
  artificial
FMSTREQPAVQEREGF

SEQ ID NO: 29 - protein, 16 aa, Artificial Sequence
  artificial
FMDNREQPGMQERESF

SEQ ID NO: 30 - protein, 16 aa, Artificial Sequence
  artificial
FMDNREQPGMQERESF

SEQ ID NO: 31 - protein, 16 aa, Artificial Sequence
  artificial
FMDNREQPGMQERESF

SEQ ID NO: 32 - protein, 16 aa, Artificial Sequence
  artificial
FMDNREQPGMQERESF

SEQ ID NO: 33 - DNA, 214396 bp, Artificial Sequence
  pINV_CS14
  misc_feature (149402)..(149453): n is a, c, g, or t
  misc_feature (154188)..(154189): n is a, c, g, or t
gtccctgatagcactgttccatcagcgctttcaggcgataatagtagaccttgggtagcctctgcccatgtgtaacttgataccccgagaacagccacgatgatctaagcggttcttatttctccggtctcagggtcggtaatgccaacagtaagtccacagtaatcaacgaagagttttttgaaccgccccggaaatcctggagactaaacgccctgagaaagaggtaaacagaatgactaaaaatacacgtttttcccccgaagtccgtcaacgggcagttcgtatggttctggaaagtcagggcgaatatgactcacaatgggcggcaatttttagccatgaaaacgtaatcaactcggtgaaccaattcaaaaaatatactttatacctgcggaagtgagaaaaccaatatattcaatacttgcactatttttaatggtgccagatatattggtactttgctcataaagagatgtatcaccttttttattagaaatgtaatttctggagccttctatataaaattttgcactgggggtgacatagtatccaatattcgcagcaacagagcagtaattttggttttttatttcatctttaaaaattgtctcagtctgatagtgtttatctgtatcagatgaacgaacccaaccactatatttcagttctgctccaaactcaaaattctcatggcggtaattagcagttaatccaatataaggtattttgaagagctgtttgtaatctattgttctttcaccactaggatgtgccccttttttattcctgttccccccgttttcactataaatataactccctcccattgcattaaaactgtaacgactctcttggtaacctgctattagtccaagtcgataatccaaattatttaataaccaacctttaagattcagctcgaattcgttagcatcacggagatgcgtattgggatgccagctctggtcggtccatacctgaggggtattggaattgttccagtctttatcaaccatgcttgctttctggttacctaaagtagtccaaccggaaactccgaaagacacttttggaattagcttccaatcgatgccacctctaacaatagtcgcattactgtatttccagtccagttgactaatttttctccccccttccttgggatgataaacgcgttcttttgttttgccactcagagatcctagactaatatccgtactgatgttgtccggtatgaaaagtggatagttagtggtagcatgtgtagtaaagatcgctggtacacaaagtgcaagtacaaggaattttaatttcatcgagaactctacttttttggttgaaatgtccatgatcttcttactttataagataaacgtcaatgccaagcattattttaattaaatatttgtttatgttaccaagaacatatcttttattataatatttattgaacttttgtaaaaataaaaaaaatacctcattgtgcgaataaagtaacgggggccatagatttctctacctaccttttggtgttcatacctgaggctgatagtttcaggattccgcgacaattctactcaccttcttgttctctcgccaatatgaaatcaatcagaaaagcgttccataagaacaacgataagacaataaattgtcatcacggctgcaatagccaacgtgcctttcagtaccaacatgacaacctcctgtatttgacgtaacgtgtgctgataaatatgtatttgtatctcaactgactcaatacaacacttaagtatagtttttcagattttgtttttcccgtgaaatattatggaaatggcagaagcaatacattgaattccgattcgagtgaatacggctagcgggcacctcactttagcactgaagccatcaacttttatttgacttatttcaatggaaagctttctcactatcagtacaggtagttcccatccttttaaagtcgagagtcagactgcgactaatacagttaaagtgaccaaactcagttatcctctacacattttacctcatgtttacttggacaattcattggtgcttttataaaagaataaatattctactaatttaatgcatcacctaaaatagtattttttataatagaactgtatctatagtatattattaaagcttaattatttattatgctttaatgtggttgtccgattaaggactaatccttgaaaacgccgtatagggcgggagttcactctattgcgataacgtaaccgtacaatattgtgcgtttcccaaattgaccactactgaactattctgggatttaaatgcaataaaatggttggttgaaggtcgtggtgatgatgttagtccttttgagaatattccagtgccagatcttacttataataaagaggcttggcagtttggataagctattgtccttattccactaatacaaactttttgtaaagtatattaaatatacttcatttcatactgaaatccccggaagtattcaatcaaacaattacaccataaaaggaagagggagccccaattaagttatcaagcatgtttatgatacctgcagggtataaattttctggctgcaagcagacacatataaaacgtctttattgatcctacccacataatatggacgcagtcgtaagtgaggctctgtgagcctacccacgtaatatggacacaggcctaagcgaggttctggttttcaaattgttccggactgaggccgccacaccaacggtgccgtcgccagcgattgtaatcgcactcgatataattaaacactgttgcccgcactatttcccgatagataaagtgttctccgtggagacattccacttaggaacaattatattgttcatggtaatgtttatatttgagtatagagattaaaatgattagtcccatcaagaatattaagaacgtatttccaataaatactgcaaatactgaatatatagtgagaaatatttatcctagagttgaacatggttactttaatgaatcaccaaacatatatggcaagaagtatatctctggtataaccagaagtatggcacaattaaaaatagaggagtttattaatgagaaatccagacgacttaattatatgaagacaatgtattctccatgtccggaagattttcagcctatttccagagacgaagccagtacaccggaaggaagctggttgacagttatatctggaaaacgtcctatggggcaattttctgtggatagtttgtaccatcctgacttacatgcattatgtgagctcccagagattagttgtaagatcttccctaaagaaaatagtgattttttgtatataattgttgtattcagaaatgattctccccaaggagaactacgggcaaatcggtttatagaactatatgatataaaaagagagattatgcaagtgttaagagatgagtcgccagagttaaagtctattaaatctgagattattattgctcgtgaaatgggagaactttttagttatgcatcagaggaaatagatagctacataaaacaaatgaatgacaggctttctcaaatcaaggctcgaatgcctgttacttaatgtatattaattgtawrmrrwkswmmmmmmmmmmwrrmmrmmrmrwwmrmswwwwwmsrrwmwmrwywwwmyrmwwmkwwwrrsrrwmmmmmmmmrwwmwmwwmrwrmrrymwwmkyryrswkwmmymwsmmmyrmwwwmrymmrwwmkrrmswrmrmwrmmwmkmwrmmrmwwmmrwmmymsmmmrymrrmmwmmmwwmrwwmwwmmkmmwmmwrmmrrmkmswwmsrwwrrrmyrwwmwmmmmrmwrwywmyrmrmmrrywmwwmswmwkyswrmwmwsrwwymrmmmmmmymrmysmyrrmmmwkmmwsmymrwrwmmwkmmwrmmmwrmwmkmkkyrrmmymwmmmmmsrmmrrrywwmmmwmrrrwkwymswwrmmrwwwmmmrmrmywwmmwrrwrrrmmwrmkkwmmrrmrwrmmmrmmmwsrmmmmrmmmmmwrrwwwwmwwryrmmmrrryyrrrmrmmmmrrmrmrrymmrsmymrmrmwymmmmrkmmmrmymwmrsymmymwmrmmrmmmmmmmmmwrmwwkrrmwmsrwmmmrkmrmmmwrmmkwwmmmrrkmrwwmrrrmwrrmmmmmwrwwmmwrmmmmmmryskwwmwrmmrryrmrwsrrmwrywrmmmrmwrmmmmrmmmmmrmwmwwwmmwrmrmmkkmmmsmmmrmmrrsrwmmmmwmkrkwmrsmkswmmrrrwwksmmwkrwrrmrmwrmwwswwrmwmmrkmmrmrmmrwrrmwrmwsmmmyrwmrywymmkwmmmwkmmwkyswmmwmymrmmmrkmwrmmrymwrrwwywwmrrrmmrrwwwwwmmmrwmwywkwmrmwrsyrmrmwrmmmmmmcgagtaggcagcctgacggctgcggcttgtcatggtctgcaattaccgttataaaaacaggcaatgtcattgtctttcaggtggttatatggcccgttcagctaaaccccgtaaacgtaaacccgcctctcaaagaagcaaacttccccgctatgtcgtgaagcttcacgaagatgatttttttgacgaagaagacgcagaagttctgcgctttgatagttttgacgatgcmrttgamwrmwrmrmmrmmmwrmmwmwkmmrwwcwwwrtggatgcgggaaacaaaaagctggtcttctggtttgtacgtgttgatgacgaagggtatccggaaatcgcccgctgcacggagcgggaatttgcgaccattctttccggtatcagcgccggcggtatgtactgcccggagtgtggcacggttcactggccggacggagtcgccccgcccttctgatgcttccccgttttgccgatatctttcagcagggaaaccgctggcttaactggctggagaagcagccggaargggcagtacgtccggtggtgattgagtcggtgaccaaaatcatggcgtgcgggaccacactgatggggtatacacagtggtgctgttcatcaccggactgttgccatacaaaaaaggtctgcttccggtgtaaaagccgctcctgcccgcactgcggggtgaaggctggcgcacagtggatacagtatctgctgagcctggtccccgactgcccgtggcagcatattgtgttcacacttccctgccagtactggtccctggtgttccacaaccggtggttactgacagagatgagccgcattgcagcggatgtgatactggaaatctgccatcaggcagatgtggagccggggatattcacggtgatccacacatgggggcgtgaccagcagtggcatccgcatatccatttatcgacaactgccggtggtgtgacgtcgggccacacctggaaaaatcttcatttttacgcccgtaaggtgatgagcatgtggcgctaccggataacacggttgctgtcacggaaataccctgacctggtgatgccggatgcgctggcagcagaaggaagcagtaaacgggaatggaatcgcttcctggacacgcattaccgccgtggctggaatgtcaacgtatcccgggtgatggataatgccacccatgtggcggtgtactttggctcttacctgaaaaagccaccagtgccgatgagtcgtctggagcattatgccggtcaggatgaaatcggtctgcgttacaacagtcaccgaacaaaacgggaagaatacctgttgatgagtggagatgagttcatggaaaggttctcctggcatgtggcggataaggggttccgtatggtgaggtactacggttttttgagtccggcgaaacggcggttactggaagaagtggtgtacatcataacggagacagtgagaaaaacggcgatgcaaatcacctggagagggatgtatcagaggttactgaaggttgacccgctgaagtgcgtgctgtgcgggagtcagatgcgttttacggggctgaagcggggctaccgtctggcagagcaggtcctgatgcatgagctgctggcccggatgcggtggtgcggctgagagccgcagaggggaagttgcgtccattttgagggaaacggagcaaaaaaccgccattcataccctgaatcaatcagagccatccagtttaatcgtcggttccgttcatatggagcaaaagtggtgttgaagaaacatccgttttgtggtgtttttttaatctttttggggttttaattcctatcgatgaacgcaacctgacaagagagagaactcaggtcgggcttcaagcagcacgagccagaggacgtaagggcggcaggccgaaaacactgagtaaggataaacaggcccttgctgttcaactctataacgagaaaaaacatacagttgcgcagatttgtgtgttgatggggatatcgaggccaacgctgtacaaatacatagagtcagccaggttgttcaagaaatgaacttagcgtacgttttcgtacggttgggcttcaaatccctaatacgttgacggggcaatatgcagttcgctgcatagcggtccgaccccgtactgctcacgcagcttatccagcagtggcatcattttttccagaggcggtcgaactccgccttcgcaaaataagcggaagcttggcgaaggatatcgttactgcggcgcagttcacgattttcacgctccagctctttcagacgctgacgttcagcggtggtgagccctccatcaccgcccccggtatcccgctcatgctggcgaacccggacacgcagagtctccggcgtacagccaatctttggagcaatggaacaaattgtcgcccattgtgagtcatattcgccctgactttccagaaccatacggactgcccgttgacggacttcgggggaaaaacgagtatttttagtcatcctgtttacctctttctcaggaagtttagtctccaggattcccggggcggttcatttgaggatcgccatgatttggctgtcggtaaattttgatgttttcataaagaatctcctgcgtttatggtacgagaaaattctacttttgacgctggttaatttacggggtgattaccagaccaatcgccagtggcgcaaagcccgcaggagcgcgtttttctgtcgctccggacaaaaaacgcactgaggacaaactcgctcagcatacaggcgcttaacgcatagccgccaggtgaatggaggccataaccattagccgcaaaaccgctggttgtcgcatcaaacccagccttaccgctggcgataccatacagcactgccgcagcggcaataccgccgataacctgagcgataatgtaaggtaacacgcgcgctgctgggaagcgtccaccggcccacagacccaaggtcaccgcggggttaaaatgcgcaccagagatgtgcccgaccgcataagccatggtgactactgtcagaccaaacgccaacgccacgcccaaaaagcctatacctaactgtgggaaagcagcagagatcaaggcgctaccacagccacccagaaccagccagaaagtgccgaaaaattcggcgctaaaaggtttaaacatacactgatgtcctgtttgttaaagagcaggcatcagggtaaaggcggtcacgatgtaccgcctagcggggatttgtaagagaggtgaagaattggtgtggaaggttaaacctaagcaatcacaggcgaatgacattgctcgcttgtgataacagcgattttttctgctgccaaactagatcgcaaatatcatcagttggattgaatgccgctggtgcttgtaacaacaaactgcgaattcgctcatgatcaaagtcatgacaagcttgatccagctctttaagtaagagcgatagatcctgccacggtagcatcacttcgttggccgtcataattcgtggatgagaggtaccttgaactgaatcaccaatcagtaattcttcatacagtttctcacctggacgtaatcccgtaacttcaatggcaatatcgccatctggatttttatcatcccgtacactcaagccacttaaccggatcatgcgtttggctaagtcataaatcttgaccggatcgcccatgtctaagacaaagacatcgccgccatgccccatcgcccccgcttgaatcaccaactgtgatgcttccggaattgtcatgaaatagcgaataatgtcacgatgagtcaaggtaactggcccaccttgggcaatctgtttttcaaacaacggcacgacagagcccgaagaaccgagtacattaccaaatcgcaccatacaaaagcgagtttggctttgccgtgcagacaaagcctgcaataccaattcggccagacgcttagttgtccccatagtgttggtcgggcgcaccgctttatcggtggaaatcaacacaaaagtttcaacgccactttcgatcgctgactcagcgcagtacaaggtaccaaacacgttattacgtaccccttccaccacattatgctcaaccagaggcacatgtttataagcggccgcatgataaaccgtttgaataccaaaggacttcatcaccatctgtaagcgattctgacgctgcaccgagcccaacagagggatcactggaacattcaaaacttctttgtcacacagcgccgagagctctttctcaatagcgtagagggcatattcagacagttcaaatagaaccaatttggccggcttatatcgaacaatttgacgacaaagctcagagccgatcgagcctcccgccccagtgaccataacggctttgccagtaatgttttcggccatcaatttggcatcaggagcaaccggatcacgacctagtaagtcatcaatcgatactttttttagattactgatttgtgctcgaccttcgactaaatcgaccatacccggaatagagagaacttcacacggtagatgctctaaacgagtaatcactttctgcctttgtgacttactgacgctcggcatcgccaaaagaattttctttataccatagcgatctacaaggtaactcagtttatccgagggataaactgttacaccatgaatgacagccttatgcaagcgaggattatcatccacaaatgccacaggaaaatattcttttgcctgcattaatgctggcaataattgtcggccagattcaccagcgccataaatcaaaacaggcgcactacccttaactccatagttaagtatcattctaaaaagcaatctagagcctgatgtcagtaaaagtaaaaccaaaaaataaattccgacaacagtgcgcggcaacattatggatagcgaatatgatataacgacaagagataacgttgaggccagaataccaactggtattagcaacattatctttgcactaacataccgtaagactgcccgataaagcccaatcctgataaatataaacacggtaaatatggtaagaatagctgcagcaaaccacatcggtgcactaagaaaagccgtttgctcatcaagcctcaaccaataagacaaccaaaatgagaatatcaccataactacatcaatgcagatgatgatacctctcttaacaattcttggcagctccaatattctatcaatcatcctacactaccttattatcgcgacaaaatcttaataatagttttccaaattatcacacaatcatactttactgaattgttagcaacataatctaaataatatctttgcttgattggaagaattatatctatataagccctacgtgggtcatcataactagacaaaatctcattttcatctaccatttctatagatgctaagtcagttatccctggcctaaccgataaaactttttcccttatatcatcaggatactcatcaataaactccctcacttccggtcttgggccaaccaaactcattgttccagaaagaacatctatcaattgaggaagctcatcgattttgtacttccgtaaataccatccaactctggttacccgagcatcttgaccaactgttatccgtccttttttttctgaatcgataaacatcgttctaaatttatgtatcctaaaatctttcccaaatctcccaactctatactgcctaaaaaaaaccccccctttagaatcagcaacaattaacattgacacaatgataaaaacaggaaatagaaaaagcaggcctaagcctgccacgataacatcaaaaatacgtttcatttaatataaaataccattacataaataattgtctaatcgatttgataacgaacaactgatcttgatccgtcattttagtataaagaggaatagatatttcatttaaataacactcctcagaaactggaaagtcactggcgttcaaattatatgtatcacgccaaaccggttgcttatgcaacggtataaaatggacagaacaaccaattccaagatctgataacttcttaataaaatcatcgcgattaatagccgaatcagtttttaagcggataggatatagatgccaagcatgaatatcactagcattagtaggccattcaggcaattcaagtggcaattcttttaacgcatcatcgtaaatttttgccattcgttgacgttttttctgaaaatcatcgatctttctaagttgatgaataccgattgccgcacagatatcaggcatattgtatttaaaccctggagctacaacctcataaaaccaagaaggagttttagactggtaccggtcaaaagcgtcacgactgattccatgtaaacgcattaccttacaacgctcaattatatctttattttttgaaacaaccattccgccttcaccggttgtcatagttttattggcgtagaagctaaaaaccgtagcatctgaatcaagcgttcctatcttacttcctttatatgttgtaggaaatgcatgagcggcatcctcgacaacctttaggtcatattttttagcaattgataagattgaatccatgtcacaagctaatccagcgaagtgtactggaataatcgcctttgtttcattagtaatagccttctcaatggcatcaactgatatattaaatgttttacgatctacatcaacaattacaggatcagcaccaaggtacctgacaatttcggcagtagcagtgaatgtatatgatgggacaataacttggtctcccggctttacgccaacagcttcaagagccaaatgtaagcccgacgtagcagagttaacagctaatgattgaacgttcgctcctaggtaattagaaaattcttgttcaaattgcttagccttaggacctgtcgtaatccaacctgaacgtaaagagtcaattacctctgcaatttcttcttcgccaatttcaggtaacgcaaaaggaataaaatttttcatttaaaataccttttcaaaaaagataataacgatccacatccataagaaatatgataagacaaaaatataaaaggaagaaatactctacctataaaacttcgcccatctcgaattgaaaacaaaatagaaataagaaaataaaaaaataaaaaagataaatatatataatcaccagtagcaaagaaaagagaaaaagatacaattaaatacaaaacaaaaaaaagtggcactatatgtctaaaataatatgctccagacataacaacccaaaaaccatcaagaaaacctttctttatgaaatctttaaaattatctctaacataatactcaacaaataaactattatctgtatataatgacaaattattgcttaaaactctcttatgaaaatcaatatcttgatttctatctaatacttcattaaaaagtccaacatcaaaaaacacatctttattatataaagcaaagacagctgtatctgttttttttagtcgattatttttgtctacgcacctaaatggagaattaccaacaccaaatttcgatgcataaaaatccgctacaattgaaccaaacttagttttataagaattagttaatacacctccaacaccaacaacgctatcatcaaactgcataatattaaaataatcatctaataatttagatatatagttatttggaatatcactatgtgcatcaactctacacacaaacttaccattagcttctagcaccccaatattccaacccgtagcaagagttctttttttattattgataattttatgcttaatgttgctatgtttagaaaaaacatcctgaacaatagatattgtattatcagatgaatccccatctacaataatcaattcaaatttatctaatccaacatcttgattaataaatgccaaaagagatttttcaatatacttctctgcattaaagcaaactataattaaagatactagaacatcactcacaaataccactcctcataaaatatagttttcttttccctgagtgaactgaatcaacaaacaaaaatctattatcaaccgagatccttggatgtaagtcacatcgagaataagaaaaaaaactcattgactcataaaataatccaagctcgcgataatcatttttgacaaggtcataaacaaacaactttttaattctatttttatctgggtaagtatcagatataatatatctattatgaactatagtaggatgtccatcagaatattttcttgcatcaaaaaacaatttctctgtattacaggattcaattgacaatctataataatttagcgaatctatttctgcaccaaaaaaacctataatttcatcattcccattccaacaacaatgactaataatcgattcatttattaggactcgaacattatcatttattaaattccaatgtattaacctttcacgctttcctttaggtgtatagtatcgatgtatgaacataacggaacgtccatcgggagcaaacataaaatgattgaccacatgttcaacatcaatattacatttaaaattagttctctttagcatttcgtctaaggataaaaataattctattccccccgtattcaaattaatgattgaaatatctgtagaaaatgattttttattataataaccataatcgctatcatatttagccagccatgagaaatctaacgtcacagccttgtaattattcacatcataaatcggatagtttattttttttataaccttcatcaaagacaaagaatagacaacactaatgtatccattattttcatagtcattaaaaattatattgtcatcatcaatccacattaatcggcttccctgctgccaattaaatgctcgcgtatcatatagcttataaactttgttattaagaaggtcttttacgcaaatagatatatacttaactttctttggatgtcgtttagtgctaaacgcactagagtggaacaatacgtaccgcccattagagctcattggactatggtcataatagccaaaaaatgattcttcagaatcatcaattgaaatctcgtatatttttgattgaataaccgtcttatttttattcaaaataattccgaataatgatgcgatatataaataaaagaacttaataacatcccttaccttaggatacttactcaaggctaaggaaatttttctttcaacatattctaacaacatattctcatcctaaatagctgcgcttaataccgctacacttttgccagcccatgtttgcctccggggaatgggctgacggtttccataaaatggcgaacttttttcaacagttgccacattgagctgcactgatgattacgcgttattgtgtcgtgaagtgcctgccatagccgttcaacatgattcatccatggcgagtaaaccggctgataaatgaccctgaacttcgggttctccttcagccagctctgtgtttcccggcttttgtggataatgtagttgtccacgatcagcgtgatggttttcgcccgacggtatgtcgctttaagccgcttcagcaggctgatgaacagcgccgaacttttgctgttgccgcccacacagctgactttacctgtcccgctgtgcagcgctccggccagataatatttttcattctgtcccggcgtgaccacccgtttttgctgtccgcgcagttgccagtccgcaccgattttgggattaagatggatatccacttcatcttcataaaagaccggatgctctgcgctgcattcgtccagtgctttatggattgctgccatcttttcatctttatgcgggtcacggatacgcagagttggcgcagcccttcgccacacaatccccgcagacggcaaccagcggcgaacggttccggcatttaactggcaaccggttatctcattgatttttattgccagcagttctgtactccagcgtgaacgctggtagccaaagtcgccgggagaatgttttaccagctcacgtaacagtgtgcagatatgctcaaacggccagcgacgggcacgcccggcaggtaatgatttcagtccctcaacacccgactgcgtgaaccagttaatccagcgtccaacagaggaacgggcgcagcagagcgttctggcaacgtcgctgacacggtcgccccggtgcagcatcagcatggcagtcagtctgcgggcataatttttatcgtgtgttttatggatggctttctgcatcaggcgtcgttcgtcacgggaaattggtgctatgatcggcattgctcagtccggttggtgatttgttttgatttggcgattgatcagatcgcacaatccgggctaagttccctcaaagtgatctactattccgcgcagctatttagaattacgtataaaaaccaccgataaaaaaatagaaatataaatatatatcgctggcgtcaatatatactcattaaaaaagacatagaaagccaaagcaaaaacgcatcctttaccaatataccctccgcatttaaaaattggagacagcattatataaaatacagaaataagcaaaataaaaccaataatccctaactgagccgaataagataaataaaaattatgcgccaacccaaagttggtacttcctaaaaactttttatctatagcctttacataatttgcaacacctaatccagtgccaattaaataattttcttttaaaacatcaaccccaataaatactaactcaactcgacgataatcattctgactttttgtaagagtgttaccaccagagaatataacctgtgacgaagacagcattcgttcaagacgagtgcctatatcagtaaaagaaatacctaaagttagaagaggaacaaaaaacaatgatattaataacttttccttctttgttaactgaagaaaaacaaaaaacaacgatatagcaaatgatattattcctgctcggctatccagaaaaagaaagaacagagagtacaatacagatgagacataccaagactttcgaccacgataaaagtatgaatatacacaataaaagcacactactataattgcggtggcatttctatttaaataaactataaaattactatcttcagaataaccaataaataattttacaactccacagataataccaaaacacattacaacatttatatttctaaccgaatgagttaaataaagctcatctttaccaatgcaaaatgacaccaaaagaaccaaagctataaaattagaaaaaaacgataataaaattacgacacctgatatatccgctgataacaacaacatctgaatgctgatataaatcaggaatatgatgcctaaaaataataatttctttgtccacagagaaacgtgaaacctcaacgttattatagatgttaaaacaacaaagaaataaaatattagatctggaatatagccaataggaactccaaaaaaacgctctaaatcagagttcatttgtccataaaaaaaaaccggaaggatattaaataacaaaagtacaggatataggtatattaacatacttaacctttattaggcttccaatcgagtctaaacatattaatggaacgatataaaaccacaacatattaatataacaaccgctgctgaccattaataataaaataaaagaaaattgggacaaagatcatcattagtgaatacttatcatgaagaattttcccacaatcttttttcacagaaataatataaaatattccaattactaatgttgcaatggcacttccaaaaaccgccccactccatccataccgaggtataaaataaaagaaaaccgccattgaaatgaccacaccaatacaatacgatttaactcgctcgctttgatttccactacacacaagataaagttcatttattagccaaaaattatatttaataagagccataaacattatcaaaaatgttgcagaagatattaccttatactcatcaccaaacaatatccctacgaaatagatgccaaaaaaataatagcataaacatagtaataagactaacagtgatatatatcgttgtaatttccttatattaccccattcagaatttttcagataataagaaaatgaaggcaataaaacagtataaataacattaaacactattattagaccggtattaaccctagttaccacctgatatatagcaacactattataagagaaatattcttttaatataaaaaggtcaatactagataaaacaacatttagtaacaccagaatagcgaacggagttgcatccaaaaaatctttataaactaaaatacttggtctttttatatgaaaacatggcacattgcatttatgtgatgagaaaaccaaaagcgaaattgagtatatgaatattgatatcgcactataatatagaggttcgataaacagagataaaaataaattacacccaatatatataattgttgagtagatatttatatataattcacatctaaaccgaccaacagcctgtagcaagctaaatctaccacttgaaatacaaacaaaatatgcagatgaaatataaacaagcagagttaatgaaattgaatcagaaacataagaataaaacatgaagattataaaaatgaagaaaaaaacatatgttttaactccatcacttcttgataataattctgatgcagaataactattatcagaaatatctttcactaattttgttctaaatccataatcagagaagttaagcacaaaccaataaacagtaaacacataactaataataccaaatgtagtaatatctgacacatgtgtgaaataaacaaaaactcctatttgaaatattgctttaagtaaaaatgtaccacctgcatcaatcatttttctaaacatacactgaccatttagaataaatacatggattttcacccctgcattaataacagaggtgaaatagattatttattcatgttaatataatttagccttttaaaaatctcacataccacggcattgaaagtcgtaatccctctctgatttttatatttggtctatactttagtaaatctatagccttagtaacatcagcctgagaatgcctaacatctccagatctaaactctctatacttaatgctcaatttatcgatatggtgaattaaattaagctcatcataaatgtaaccagataattcatttaacgttgttctatcaccaactgcaacattatatatattatctttagcactgtccttcgctaatgcagataatatattcatttgtataacattatctatataacaaaaatcacgactcgtttcaccatcgccattaatatatacgtcatcacctttaagcattgctgctgtccattttggaattactgcagcataagctccattaggatcttgacgacgaccaaatacattaaaataacgtaatccaatagttttaaaaccatatgttcgagcatatacctgagcataaatctcgttaacatatttagtaactgcataaggagaaagtggattaccaatgttttcctctacttttggtagtgcgggatgatctccataagttgagcttgatgcagcataagtaaaactttgtacttgtgcatttttagccgcatgtaagatattcaaaaatccagtaatattagttgcattggttgttataggatcaacaattgaacgaggtacagaacctagcgcagcctgatgtaagacatgatcaacacctttcataacttgctcacaggtagtgagatctcgaatatcaccttctataaagcaaaatcgactccactgttcagtggaaactaatgttttaacttcatcaagattatattgatgtcccgtggaaaagttatctaacccaataacaacctggtttaatttaagtaacttttctaaaagatttgatcctataaagccagcgacaccagtaattaaccaagttttcggtgaaaaaattaactgctgtgtaatctcttcatagcgagacatataaatatccatgtatattaatcaagtgctaaacctagcataaaatgctgaggtgtatcatatcaaagaggatttgatttttttaatcactcttacaagcgcatatcgatactttttttatcaagcacatattttaaatcgaacaaaacgtgctcgtctttacctaatgcacgaatagcgctctctcccatctcgcgaaactcattgtgtgcaacagcgataattattgcatcataatgattaactttaggatcttcacataaagttaatccatattcatgttgtgcctcatcggtagaacaccatggatctataatatctatattgatattgtattcttttaactctgaaataatatcaatcactttagtgtttcgtagatctgggcaattctctttaaatgtaagccccatcactaacacattcgccccttcaacttgaatccgttgtttcaacatttttttgactaactgggaaacgacatactgccccatactatcatttaaacgacgtccggctaaaatcatctccggatgatagccgacactttgcgctttatgtgtaagataataaggatctacacctatacagtggccacctactaaaccgggcctaaaaggtaaaaaattccacttcgtacctgcagcctcaagaacctctaaggtatcaatccctaacttattaaatataatagataactcattaatcaatgcaatattgacatctcgctgcgtgttttcaattacttttgcagcctctgctacttttatcgatgatgctttatgcgtaccgacagtaattattaatttatatagctgatctacatactcagcaacatccggtgtagatccactggtcaccttaaggatattagttacacgatgctctttatccccaggattaatacgctcaggtgaataaccggcaaaaaaatcaatattaaacttaagacctgatactttctctacaactggtatacaatcttcttctgtcgctccagggtaaactgttgactcataaataataacatcgcctttctttattatcttacccaatgtttcagatgctttaattagaggtgttagatctggctgtttatgtttatcaattggagttggaacagttacaataaatacattacactcttttagtgcatctaatgaacaggcgtacgtcaatttagttgcttctaacaactctaaatttgagcactctaatgtactatcgtgaccatttcgtaattcatcaatacgagacttattaatatcaaatccaatcgtcgttactttctttccaaactcaacagcaagaggcaatccaacataaccaaggcctataatcccaattttcgcattcaaagtatcgaatttcattttcttatccatcataaagtgcacatagaacatctatttcgcattgacacaacaatacgtaacccagtttaatctaacttggttttatcttttaatgtcgcataaactaatgtaccaactatagcaaacattgcccctggtaatgcaccaagaatcaaaattaatgctttgttgggtttgtcttttataactggctcagatggcttcatttgatatcgatatgcttgcgcctgaatgttatcgcgtaacattccttctaactcatcaatttgacgtttcacttcgtaatatctaaatgggtaaataattggcgcatctttagttgcttttagctctgctgataacagtttttcgccgagcaagaacttatagctactttgcgcattatcgaagccgcctttgctagagcgataatcaatgattccggcttgttccgcagttgttaaatctcgctgaatatgagctatttgatcaagcttttctgattttctctggaaatctatgtcaataatttctttatttagattaaccaaaagagtattaacattttgaccaaactcatcaagacttaatgttacagcttgattattaacattctttagatatccctgcagtagtttatatgcctcttgcgctgagtctgccgatgcttgtattatgtagtaacttgtatttcccttttcctttggtggcgtaatcacaagacctttattcgcttgctcagctaaccaaattttcttatcttgttcagtagataaattatttgtttcctttttataatactcagtactctctagaaatttaactttggtgtctggcgattcagcaatcaaaatcaaatcggaaaacaatccattagccaacgcattcgtatctaacggttgatttaatattcgggcatacgcttggtgaaatttaagatagctgcctaattcagcaatacgtggtgcttgaatataggttttagccgtccactgctcttttgcggtaaaggcatacactgctgctaaccccgtagtcagcaacgtgacacataaaataacccattttttcttccacaaggtgcctaaaagctcgaaaagatcgatttcatcctctgcttttggcatataagttggatagaccccttgcgggatcagataaggggtctgttgtggttcagatgcttttgacatgaaaccaatccattatttatcaatgggttaaaaataaaaatgaagaataagcctgacaaaaaagtacgctaccgcccttgagtttcagctcccaaagcgctgcgcagaaacaggatatagccaactgcgatacaacaagtgatatctgggattagaggacataatcgagaacactccgtgtgaagagacatatacagtcccacacttccctgctgcaaaccggcggatagcccgcagaatttggagtgcagattataaagcaatgcctgtataagaccaaatctgccttcaaaatgataacagtatgtttgtaaattctgtgaggctaaatagctgcgcttaataccgctacacttttgccagcccatgtttgcctccggggaatgggctgacggtttccataaaatggcgaacttttttcaacagttgccacattgagctgcactgatgattacgcgttattgtgtcgtgaagtgcctgccatagccgttcaacatggttcacccatggcgagtaaaccggctgataaatgaccctgaaccacggcagcctggcggctgcaaatcggggtgtggcagaatatgagctttcagagtaaccatccagaatgagttcccatgtcacacaacctggagcaccagaaagtccacaccagaatggtgaaagaggttctgaaagccgtcgccagagccaataatcacccttatcagtccgtatttgcagactttatcaccggtcatccctcctgcacggtctgtttctgggaaaccttccacaaaatgtacccggattcaccgtacgaatacgtcacgttctgccatacctgccgtcgttttgatttatacgaaacagaagcggagatgaaggcggatgacccgaagtggtggtgattttcagccccgaccgctgaaacgcctgttcacagccaaccagtgctggacgtccttcctggatgcgggcggtctgcgcgatatcgaggttgaggctgtcacaaaaatgctggcctgcggcacgcggatactgggcgtgaaggaatacaactgcgataaacctgagtgcccccacgtcagatacgtcactaactcatgcggcagccgtgcctgcccgtcctgcggaaaaaaggccacagacctgtggatagcgacacagctgaatcgtcttcctgactgcgactgggtacatctggtcttcaccctgccggacacgctgtggccggtgttcgaaagcaaccggtggctgctgaatgacgtgtgccgtctggcggtggagaatctgctgtatgccgcccggaaacgggggcaggaacccggtatcttctgcgccatccacacgtatggccgtcgtctcaactggcatccgcatgtacatgtgtctgtaacctgtggatgtctgaataagcatggtcagtggaaaaagctgagcttcctgaaagacgcgatgcgttcgcggtggatgtggaatatgcggcaacggcttctgaaagcgtggtcagaggggctggcgatgccggagtcgttgtcacatatcacgacggaatcacagtggagaagcctggtgctgaaagccggaggaaaatactggcatgtgtacatgtcgaaaaaaacggccggcgggaggaatacggcgcgctacctgggtcgttatctgaagaagccgccgatagcggcctcccggctggcacattacaacggaggggcgagcctgagcttccgttacctggaccacaaaacgggagaaacggcgacggaaacgctgacacagcgtgagctggtcgcgaggctgaaacagcacatcccggagaagttttttaagatggtgaggtacttcgggttccttgccaaccgggtgtgtggagagaagctgccgcaggtgtaccgtgctctggggatggataaaccggaaccagtggcgaaagtgtgctatgtacaaatggtgaaacagttcctgagtcgtgacccgttcgaatgcgtgctgtgtggcggccggatggtataccgccgggccattgcggggctgaatgtggaggggctgaagaaaaacgcgcgggatatcagtctgctgaggtatatgccggcctgacacaggctaagtgtgcccgggaatcggtgcaagtataaaaaacagcctgttgcggtataagaatcgaccatgcagagagagaaaacaccggagtggtggggaaaaacagaaaggctcacgtggcataaggcgcggacaggggtatcgcttagtttttcaatttcctatacgtgaacttcgggttctccttcagccagctctgtgtttcccggcttttgtggataatgtagttgtccacgatcagcgtgatggttttcgcccgacggtatgtcgctttaagccgcttcagcaggctgatcgagtaggcagcctgacggctgcggcttgtcatggtctgcaattaccgttataaaaacaggcaatgtcattgtctttcaggtggttatatggcccgttcagctaaaccccgtaaacgtaaacccgcctctcaaagaagcaaacttccccgctatgtcgtgaagcttcacgaagatgatttttttgacgaagaagacgcagaagttctgcgctttgatagttttgacgatgccgttgaatgctgcgcagacctgaatattccgtgaggtagcctgagtttaacggacactccttcctgaaatagaatggcatcagaaggagctaataatgagcagaaaaacccaacgttactctaaagagttcaaagccgaagctgtcagaacggttcttgaaaatcaactttcgatcagtgaaggcgcttcccgattatctcttcctgaaggcactttaggacaatgggttaccgccgccagaaaagggctcggtactcctggttcccgcacggtggctgaactggaatctgaaattctgcaactgcgtaaggcgttaaatgaagctcgccttgagcgagatatattaaaaaaagcaacagcgtattttgcacaggagtcgctgaaaaatacgcgttaatcgaacaatggcgacaacaatttcccattgaagcgatgtgtcaggtatttggtgtatccaggagcggttattacaactgggtacagcatgaaccctcagacagaaaacaaagtgatgagcggctaaaactggagattaaggtggcacatatccgcactcgcgaaacatatggaacccggcggctccagacggagctggcagagaatggcatcatcgttggtcgtgaccgactggcacgtcttcgtaaggagctaaggctacgctgtaagcagaaacgcaagttcagagcgactacgaactcgaaccacaatctgccagttgcgccaaatctgctgaaccagacgttcactcctacagcaccaaatcaggtctgggtggcggacctgacgtatgttgccacacaggagggatggttgtacctcgctggcatcaaagatgtttatacgtgcgaaattgtcggctacgccatgggagagcgcatgacaaaagagctgacaggtaaagccctgtttatggcgctcaggagccagcgcccacctgccgggctaatccaccactctgatcgaggttcacagtactgcgcatacgattaccgggtcatacaggagcagtctggtctgaaaacatcaatgtcgcgtaaaggtaactgttacgacaacgctccgatggaaagcttctggggaacgctgaaaaatgagagcctgagccactatcgttttaataaccgggatgaagccatctcagtaatacgggaatacattgagattttctacaatcgtcagcgtcgtcactctcgtctggggaatatctccccggcagccttcagggaaaaatatcatcagatggctgcttaaaaaaagaacaaatggtagtgtccgctattgccagtacacctcaccgttctwtrtggatgcgggaaacaaaaagctggtcttctggtttgtacgtgttgatgacgaagggtatccggaaatcgcccgctgcacggagcgggaatttgcgaccattctttccggtatcagcgccggcggtatgtactgcccggagtgtggcacggttcactggccggacggagtcgccccgcccttctgatgcttccccgttttgccgatatctttcagcagggaaaccgctggcttaactggctggagaagcagccggaaggggcagtacgtccggtggtgattgagtcggtgaccaaaatcatggcgtgcgggaccacactgatggggtatacacagtggtgctgttcatcaccggactgttgccatacaaaaaaggtctgcttccggtgtaaaagccgctcctgcccgcactgcggggtgaaggctggcgcacagtggatacagtatctgctgagcctggtccccgactgcccgtggcagcatattgtgttcacacttccctgccagtactggtccctggtgttccacaaccggtggttactgacagagatgagccgcattgcagcggatgtgatactggaaatctgccatcarrmmrmyrwrrmrwmrrrrmwmwwmacggtgatccacacatgggggcgtgaccagcagtggcatccgcatatccatttatcgacaactgccggtggtgtgacgtcgggccacacctggaaaaatcttcatttttacgcccgtaaggtgatgagcatgtggcgctaccggataacacggttgctgtcacggaaataccctgacctggtgatgccggatgcgctggcagcagaaggaagcagtaaacgggaatggaatcgcttcctggacacgcattaccgccgtggctggaatgtcaacgtatcccgggtgatggataatgccacccatgtggcggtgtactttggctcttacctgaaaaagccaccagtgccgatgagtcgtctggagcattatgccggtcaggatgaaatcggtctgcgttacaacagtcaccgaacaaaacgggaagaatacctgttgatgagtggagatgagttcatggaaaggttctcctggcatgtggcggataaggggttccgtatggtgaggtactacggttttttgagtccggcgaaacggcggttactggaagaagtggtgtacatcataacggagacagtgagaaaaacggcgatgcaaatcacctggagagggatgtatcagaggttactgaaggttgacccgctgaagtgcgtgctgtgcgggagtcagatgcgttttacggggctgaagcggggctaccgtctggcagagcaggtcctgatgcatgagctgctggcccggatgcggtggtgcggctgagagccgcagaggggaagttgcgtccattttgagggaaacggagcaaaaaaccgccattcataccctgaatcaatcagagccatccagtttaatcgtcggttccgttcatatggagcaaaagtggtgttgaagaaacatccgttttgtggtgtttttttaatctttttggggttttaattcctatcgatcaagcggctggcacgtaaaacaatctgcttctcgcgttccgtggaaatccacgaaaagtcatcggctccttcattgaaaaacacatattctactgattggaagcgccacccgaaaaagtatcgtttttttacagtaaacttcatttagccgactataatgtaaaaaaataaattttttaccaaaatataacataaggaagtaatatgcttacacaaactatattcccttgtcttcccaaaaaataagaaaatatcatccctgaagtatcaaatactatattattatcttctactgttactacagatggatatactgtttttaataaaaaaaacagccatatacgaattgcaaatacctgctaccaaccgaacgaaaaggttgaaattcacagctacagaaatacaatggttgacaaagataaatgaagccggaattgataaaaaacaaagccaacggtattctgatttctgaagttaatttcagactcgtttaagctctattttcaaaaaaatatatcagggtttgttactaataacttctgaaaacagaaaaaaatagagcagtaattctatttcagagaggagtaactccatctatcctcgatacgtactgaacgttatcgtgatatattcatcatgctttcttcaccattattcgagtacagggagagacgcaatgaccttaaatacctttttatggctgaattaccattctcaggaataacagcagactccttcccatagacggcttagggggaaagcaatacccggagaaagggtacaaagcaaactgttttaaaattaataagctatattctatttcactagaattcttatcattgataagaaattttagttataatgattaaatcaaccaatatacaggcaatcggttctagtattatgcatcaaataaacaatatatactcgttaactccattttctttacctatggaactgactccatcttgtaatgaattttatttaaaagcctggagtgaatgggaaaagaacggtaccccaggcgagcaacgcaatatcgccttcaataggctgaaaatatgtttacaaaatcaagaggcagaattaaatttatctgagttagatttaaaaacattaccagatttaccgcctcagataacaacactggaaataagaaaaaacctattaacacatctccctgatttaccaccaatgcttaaggtaatacatgctcaatttaatcaactggaaagcttacctgccttacccgagacgttagaagagcttaatgcgggtgataacaagataaaagaattaccatttcttcctgaaaatctaactcatttacgggttcataataaccgattgcatattctgccactattgccaccggaactaaaattactggtagtttctggaaacagattagacagcattcccccctttccagataagcttgaagggctggctctggctaataattttatagaacaactaccggaattaccttttagtatgaacagggctgtgctaatgaataataatctgacaacacttccggaaagtgtcctgagattagctcagaatgccttcgtaaatgttgcaggtaatccattgtctggccataccatgcgtacactacaacaaataaccaccggaccagattattctggtcctcgaatatttttctctatgggaaattctgccacaatttccgctccagaacactccctggctgatgccgtgacagcatggttcccggaaaacaaacaatctgatgtatcacagatatggcatgcttttgaacatgaagagcatgccaacaccttttccgcgttccttgaccgcctttccgataccgtctctgcacgcaatacctccggattccgtgaacaggtcgctgcatggctggaaaaactcagtgcctctgcggagcttcgacagcagtctttcgctgttgctgctgatgccactgagagctgtgaggaccgtgtcgcgctcacatggaacaatctccggaaaaccctcctggtccatcaggcatcagaaggccttttcgataatgataccggcgctctgctctccctgggcagggaaatgttccgcctcgaaattctggaggacattgcccgggataaagtcagaactctccattttgtggatgagatagaagtctacctggccttccagaccatgctcgcagagaaacttcagctctccactgccgtgaaggaaatgcgtttctatggcgtgtcgggagtgacagcaaatgacctccgcactgccgaagccatggtcagaagccgtgaagagaatgaatttacggactggttctccctctggggaccatggcatgctgtactgaagcgtacggaagctgaccgctgggcgcaggcagaagagcagaagtatgagatgctggagaatgagtactctcagagggtggctgaccggctgaaagcatcaggtctgagcggtgatgcggatgcggagagggaagccggtgcacaggtgatgcgtgagactgaacagcagatttaccgtcagctgactgacgaggtactggccctgcgattgtctgaaaacggctcaaatcatatcgcataacccactgatmmtgtttactaccggatgtagcttttttcatgcccgagatggtcagttagttccgcattaagcaccgtttcgacagttaatttcgtctgcatacgggaaaactgattgaggtcggcctcggttttgaggcctttagccagttcagccgcaagggctttaagcttcttttcgtccataatttacctgtctccgttgttggagtgaacatatcaaaaacaggcaattacacaatttaaattacagtctcaaaacaaaaccaactgtcaataatagtttaccagcaagtttactcgcagagtgccaggctgtgtcccttaattacgtaagcgttgataaaagagtcggatgcagcccagtttcaccatcgccaggtaatttctcgccgttttgtcgtaacgcgtggcgatgcggcgatattctttcagccgcccaaagcaccgttcaacgacattacgattgcagtacgcatcacgatgaaccacggcagcctgacggctgcaaattgaaaaccgcccctgttcgtcctgttgttgccgcacgctccgcagaatatgtatgctctgaggacctgttttattaaggactcatcctcttatgttgacccgcaaaagtatagataccgttctgctctctgttggtgctgagaagctctcacaacgtgaatgggactggatgaagatgctcaaacccatggacccaccacctgcgatggtcgcggcctccatcctggagcgccgtggcgacacagcagcccttacccggctacaggataccgggggttaatccgtcctcatgttgtccgcttttactccccgcccgctgaaacgcctgttcacagccaaccagtgctggacgtccttcctggatgcgggcggtctgcgcgatatcgaggttgaggctgtcacaaaaatgctggcctgcggcacgcggatactgggagtgaaggaatacaactgcgataaacctgagtgcccccacgtcagatacgtcactaactcatgcggcagccgtgcctgcccgtcctgcggaaaaaaggccacagacctgtggatagcgacacagctgaatcgtcttcctgactgcgactgggtacatctggtcttcaccctgccggacacgctgtggccggtgttcgaaagcaaccggtggctgctgaatgacgtgtgccgtctggcggtggagaatctgctgtatgccgcccggaaacgggggcaggaacccggtatcttctgcgccatccacacgtatggccgtcgtctcaactggcatccgcatgtacatgtgtctgtaacctgtggatgtctgaataagcatggtcagtggaaaaagctgagcttcctgaaagacgcgatgcgttcgcggtggatgtggaatatgcggcaacggcttctgaaagcgtggtcagaggggctggcgatgccggagtcgttgtcacatatcacgacggaatcacagtggagaagcctggtgctgaaagccggaggaaaatactggcatgtgtacatgtcgaaaaaaacggccggcgggaggaatacggcgcgctacctgggtcgttatctgaagaagccgccgatagcggcctcccggctggcacattacaacggaggggcgagcctgagcttccgttacctggaccacaaaacgggagaaacggcgacggaaacgctgacacagcgtgagctggtcgcgaggctgaaacagcacatcccggagaagttttttaagatggtgaggtacttcgggttccttgccaaccgggtgtgtggagagaagctgccgcaggtgtaccgtgctctggggatggataaaccggaaccagtggcgaaagtgtgctatgcacaaatggtgaaacagttcctgagtcgtgacccgttcgaatgcgtgctgtgtggcggccggatggtataccgccgggccattgcggggctgaatgtggaggggctgaagaaaaacgcgcgggatatcagtctgctgaggtatatgccggcctgacacaggctaagtgtgcccgggaatcggtgcaagtataaaaaacagcctgttgcggtataagaatcgaccatgcagagagagaaaacaccggagtggtggggaaaaacagaaaggctcacgtggcataaggcgcggacaggggtatcgcttagtttttcaatttcctatacgtcgagtccacagagaaaataacaattctgagttatctgcgggaaaggttatttatatgttactatctcattctctcactggcgagattataaacgttactacgttctcgttttcctaccgcaacaaccgcaataattaacatatcgtcaatcacctgataaaccagacgaaatcccgatgcacgtagtttaattttgtagcagtcctttagccctcttagttttgccgacggtatatgaggattttcgctgcatttttttagttttttcacaaactgttgttgaatggtcttatccagtttcagccattccttgagcgcatcatccctgaattttaccgtataagtcataaataactgtccaggtccacatcatgcagtggctgattgctgcgttcagtaaccagtttcaccagttcctggtcatccagagcatcaagcatcctttcatacagttcagcaggcacacagtagaatgccggctgattccggttaagaatggcgacagggtaaccatctccggcactgactgtagccataggattttttttcagttcactgacacttgcactggtctcagacagaatgatgttaggcataacgcacctttgagactgtttaacaagtcttaacgatatccctgaaacgaccacttaacaagtctttattactcacacttttgcaaatgcattaaatgctgcttgtattattctgatgccgcgccagcggcatgtaaggcggcacgctgcggttacatgtgataccggagtaaaaccgccgaagcccggcgtwagccggtactgattgayagatttcaccttacccatccccagccctgctaagccatacccgttttcagccatgacagagcttctgtgcgcggtcggagtggtcccgacgagggtttacccgaagtcggggcgtatctccgcgttagcgggccgtsagggccgcttacgagcgtgtactcratacktccagcsagaagactgacagcgatgacgrwgtagttacaacattcataattaaaagcgaykytgttcccgcccttygggcgggggcrgcskcgcttttcagttatgaggaggggcgttgtggtttcagttctgcgtcggacttgggttttttaggggtttatgtttgcatgttgtaactaaatcggcttcggtcggggcccgccgcttgcggtgggaggtgcatatctgtctgtccacaggacaggcagtgaacaggttttctttttaaatggatgtaattaagtagtttaaaggagatataaacagctgtttaaaagatacattgcaccctgtaaagctaacggctggcgctttatgacatgaacggttgtaaccttatggggaagtcccttgcagtttaatgtggataagcaaaattccccgtctgtgaggcgtgttttgtatcaaaaacaggggggaccggatgcaccagaaggtggatgatgaggttgttttttgtatgtagcgctgattttttgtgcactggcgggcttcaggcgtgcgaatgcctccggcgcgtgccggattattctgaggaggtcactttcagggggaagctgtggccagccggctgtaattacggttacgtgacagaatcatgcgctccttcacacgacgctccacttcacgttttaccgcctcacgactggcagtgaagcgcccttccgagatttcacgcgtcagttgccgtttcaccagggtgacaatatcctgacgctgcctgtccgcatcacgacgcgcacgggcacgcttcattccacgggacttaagctctgtctggtaactgcggaaacgctcacgcacaaaacgccaggctttcgccatcagttcatccatacccagggtatccagtccctgcttcttgcgctgtctgttttcccattccacacgactgcggcgcgcggacgccaccgcctcctctgatacatcgagggcggcaaacagcgccggtgtgaacgtgatgtcggtcggaatgttgcagccgataagcgggtcatattccgtctggtaggtaatcagccccagctctgccaggaacgtcagggcacgcgtggcacgggtgatggaaagtgttccggcatcagactccgtcgccagtccgcactcaatggccagcgtggtgatggagcactgaacgcggttggccagcgggtcatagtggaaacacagcccctgcagcagcgcatcaatggcccggcgacgtagtaccggtggcatgcgccgacgcagaccaagcgaacgggcatgtgccacatgaatggcgaaatcaaaacgggaggtgaagcccaccgccttttccatcagtttcccgcagaacggaagcgtccgggcaccttcacggggtgtgaacaccggattcgggttctttacctggcggtaataagtttgttgaagatcagtcacaccatcctgcacttacaatgcgcagaaggagcgagcacagaaagaagtcttgaacttttccgggcatataactatactccccgcatagcttgattgttggctatacggtttaagtgggccccggtaatctttctgactcgccaaagttaaagaagattatcggggtttttgcttttctggctcctgtacatccacatcagaaccagttccctgccaccttatggcgtggccagccataaaattccttaaacgatcagtaatctagcatgctacgccacaaagtaaagtctttactttagtatatccagtctctgcagttcatctttgatgattttctcaacgaactgagcctgtgttatcccctctctctcgcagtactcaaccatgagatctttcagaggatttttgacaaaaacttttatctctttgtgtgtaagacgttttcttgcaactgcggccatttgtttctcagagtcagtcataggcttacctctgcgcacaaaccgcttttgactcaatgaggaagtcactgcattttctgtctgcgacatctcgcctcctcaatactcaaacagggatcgtttcgcagaggatactacagttttttgaaatcagcgacttgagaattgtggcgaagatctgtgttggcgcacaaatcaacggggattactgtcgtttaatgtgatttaaactgtgaaatagtatggttttcagttatggaaacgccgggagcgaggaaaacttgctttttcccgtttccggggttggacaactaagcaacgcgaaggcgtcagctacgatgttccggtgactgctgatcagcccctgttgtattcagccattctccggtcatctgcagcagttttctggcatcgtggcttctgagcaacggaataccagcctgcctcagaacctcttccagaagaatatcccggcgtctgcgttccggtcgtaaatggctggcatcatccagctcwamwrmwrmmmmmmwmmwrmmmrmmmrmmrmwmmmmrmwmmmmmmmwmmmymwrmmmmwrmrmmmmmmkmmwrmmwmmmwrmmmmmmmwrkmrwrmwmrwrrmmrrmwmwwmmmrwkmmkmwrrmmrmwmwmmrmrmmmmrwmmmwrmrsmmmmmrmwmmmmmmryttcggtgatgctgccaacttactgatttagtgtatgatggtgtttttgaggtgctccagtggcttctgtttctatcagctgtccctcctgttcagctactgacggggtggtgcgtaacggcaaaagcactgccggacatcagcgctatctctgctctcactgccgtaaaacatggcaactgcagttcacttacaccgcttcymaacccggtacgcaccagaaaatcattgatatggccatgaatggcgttggatgccgggcaactgcacgcattatgggcgttggcctcaacacgattttacgtcacttaaaaaactcaggccgcagtcggtaacctcgcgcatacagccgggcagtgacgtcatcgtctgcgcggaaatggacgaacagtggggctatgtcggggctaaatcgcgccagcgctggctgttttacgcgtatgacagtctccggaagacggttgttgcgcacgtattcggtgaacgcactatggcgacgctggggcgtcttatgagcctgctgtcaccctttgacgtggtgatatggatgacggatggctggccgctgtatgaatcccgcctgaagggaaagctgcacgtaatcagcaagcgatatacgcagcgaattgagcggcataacctgaatctgaggcagcacctggcacggctgggacggaagtcgctgtcgttctcaaaatcggtggagctgcatgacaaagtcatcgggcattatctgaacataaaacactatcaataagttggagymmwwrmmmmymkmwwmmwmwmmmmmmwwmwrmmmmrwmswwymmwmmmmmwrrmwwmmmwwwmwkwmrrmmrmrwmwwmwrmmrwwrmymrwmwwmammrmwrrmmmrwmwwmrmwmwrmrwmammrrtmmgwatacmtgccgcatgcaattgcatcagcaaaggagaatgctgatttacacttctgaggataatccggataagtagcgctgcaataatcagaacaatgagaataaaaataatcaacttcattatttatctgctcctggcaattaattattgctgaattattaccttacatatttccatatctcttttggtatcctgtcatataactttccggtgattaattccgctaaccggtggtctgctgctgaattaaatgattcacgttcactgtttgataaactgtctctgtttttatgaataattttttccagcgtattgactgatgaacatcgccgtaactgatacagccattcctgttttgtttttgccatttttattcccgctgagaacagatgaatgataggattgccatcgctttatttacggtgcatccatatccaggggggaaccggatcagcatctgaccagagcccacgtttttgttgtctggcttcgttctgaaggacaggtagcacgggatctgtgttgtactgctcatatacccaggcagccccggcccggaccataaactgattaatattcattccgtctggtgcataaacctgcccgagaatacgaccataacgatctcgctgaaaatatgtgacggtaaccgtttttcctgcgaccagtgatttcatcatgtctgttgaccaccggccataatcctgtttcttttccggtgcatcaatatttaccagcctgatccgcaccgccttcagtgagtccatgacctcgatggtatcaccgtccagtacacgaacaacccgtccatgaatatctgcagacaataccggccatgaaaagataaataataccagtggaatatatttcctcawgttmwwmtmsmwmwmmrmwmmmwrrmmymyrwrwwwmmrscmycctgaatakkgcagcmcacaytgaatmacamcttattttgtgaaattaatcacaaaatataaaawmwcgacttaatrcggaaaaractctgtgctactcagaactgtccggtcatactgcccgaccggatattaaraagcaaatctmataaactcctttcagccttctgactcctggagtaatgaaggctgcttttttcgttacacccttccccggcagcgccggtggmagtgttmagccctwwwtcgggcaaactgctgccagtctgtmmtrrrmrmmmmmmargcarmmcgtmgsmawgmttmtgmmgmttmmatmmmgmrwmmmmwramwtmmmrmmtmamrgmmcgmmmgmmrrmamrmkrrtmmmgmmmrammwrrmmrwwmmrwrmmmwgkwwmwrmmymmmywmmwwwwmwwgtcmccctttagttgtcagtattattgtgataaataattcmgtggactggctwmmtwwcggaagcgramrtwawwwcmwwwwamrmmwwawwwmmmwrmmwmmtmtmwgatggargctwtwtwwtatttctcatcaagcacggcctgaagttctgcctttatccggttctggcggcggattttatccagacgcgctgccgcatacgcttcytcctcctgagaaatatgctccgtcacatacccttccgtgtcataccggcaggcaccggctttcatggcacacagatagctttctgaacgggtgatggcmttcagcgccctgcgcagtwwtttatgcgagagcgggatawtcmgctrmgccacgtcttccagtaacacgtcccggataccgcaggccagaagccggggggtgtcaccgtcaaataatcccggccaccagggtttcagggtawtcamggcctcatcmagmgtgggcaggttmarmwmmmwrrmmmrtrmmwkwmwrgmmtgcgcwwwwwwwkmmrmmmrmwmwrmwwmmmrrrmwrmwwwmwmggmmmrcwwswgctwwwtcaccttccactttggtggcgtggtgammwwrmwratgrttttccrgctgcrramarrmrtmtmmmmwtmmrwwwkmmrmwwmmrwrwmmrwmmmrrwmrmwwswrmkmwrwmmwrmwrwrwwmwrmmwmwrmmrmmwmmwmmwrwwmmrrwmmmmrmwammmyammsrwmmmagmgcmagaacgcccagcacagccagatgacaggcgtagaataccgggaaaaaatccccgggccagaaacggggtaatgattttcctgcacacgacaccagaccaacggtcagcaccgtcatcacaagaccggccaccgccgctgcatcactggtggcaaggttaagcgccggaataacagccagcaggcaggcgaccagcgccagacgttccgctctgtcttccgcccggtacagtcggtggctgactgccagcatcagcagcccggcaatgccgtaactggtgccggacaaaggcccccaacagggccatcagaagaatggccgctgctgtacgccacmmactgcgcgtwtmacammacgtcagcammtgtrsmrmmmmwrmmmmrrmmmmmmrrmwmwwmmmmwmmwmmmmrrrmmmrmmwrmmwrmwmmwmwrmrmmmwrrrmmmwmmwwmmmmmtmmmmmmmrmmrrwwmmwrrmmrrwwsmmkrmwmwrcgmmwrmmrrrrcmrmwwmmrmmmmmacaccagggcmammagcrrmmmrrmwmmwmrmmmrrmmmrmmmmmwmmmwwmmwrmwwmmrmwrrmmwrwmmrmwwrmwrwrrwmmmrwmmmmwmmrmmmmmrwrmmmmmrwwwwrmwrmwmwmmmrmwrtmmrrrtrmmmmrmmmmrmmmwrmmwrmmmmmmrrymwmrrwmmrkrmmrmmrrwrmmwmrwwmmrwrwmrwrwwmrwrwkmwmmrkwrwmmwmmrwrwwwrwmwwmmwrwmwmmrymwmmrmmmrrrrwwwwmwmwwtmtgmmrrtcamgmammmwmtmmmgmtmmatctgctgcagamgctcctgcagcagattttcmcgggcaacctcccggaccgcttcccgctcccgttgtgattcacgcagcacactctccggcagcgcggcttcccgttcagttatggcagcccggtcacgctcctgcccggcaatttccmtgacagccagctctgwwttgccgtccggcarrtmmrrwwwrwwmymwrmmawwwwamrrmmmmwwwmmwmtrmrmrmmrwwmmrwwwmmmrrmsrmwrrmmwmwwmarmmtrmmrmwrwrmmmrwammtmwrmmrwrammrgtwcgccatttcmggctcccggctggacgctgttgtccgggatatcccccmacamgcrgccatcggtgatrgcacmrrrawwmmmmrramrrmmwwmmmmrrmmrwrmwwmmymmmmmmmmmmwrwmmrrmwwmwmwmrrrmmmwmmrrmmrmmmwmmwrmmwmwwmwmmrmmmrmmrrmwwwmwmmrwwmmrrmwrmmmtrmarrrmmamrammwrmrcmwmmmmgmtgcmtwwwacmcgacmttcaccgctgaatccmmgmagcccgwwtcmgtcatccgtggtcagcgrrttcarccasawammrrmrrmmwwrmmrwwmmrrwmmmmmrmmrrmmrwrsmmmmwmmrrwwrmrsmwmwywmmrwmmmrkmrmmmwmmmmmrwrmrrractrtmtmmmmcggccagcmccgcctgacggagaacggcacggmmtgmmrmmamrtmmmrmarmtmmmgmgmmgtaytrmammsmmgmwmwrmmwwmmwrmmmwmmmrrwmmrrwwwmrrmwmmmmmmmmwmrwrrrmmrwwmmwwwykrwmmrrmmwwrwwrmwrrmmwmmrwmmmrmmmwrmmrrywmwmrrwrwmmmmmwrmwmmwrmwrmwwmmwrmrmkmmmrrrmmmmrwmsrmwrmmwmmmmrmmrrsmmtcatgaggtgtactggcaatagcggacactaccatttgttctttttttaagcagccatctgatgatatttttccctgaaggctgccggggagatattccccagacgagagtgacgacgctgacgattgtagaaaatctcaatgtattcccgtattactgagatggcttcatcccggttattaaaacgatagtggctcaggctctcatttttcagcgttccccagaagctttccatcggagcgttgtcgtaacagttacctttacgcgacattgatgttttcagaccagactgctcctgtatgacccggtaatcgtatgcgcagtactgtgaacctcgatcagagtggtggattagcccggcaggtgggcgctggctcctgagcgccataaacagggctttacctgtcagctcttttgtcatgcgctctcccatggcgtagccgacaatttcgcacgtataaacatctttgatgccagcgaggtacaaccatccctcctgtgtggcaacatacgtcaggtccgccacccagacctgatttggtgctgtaggagcgaacgtctggttcagcagatttggcgcaactggcagattgtggttcgagttcgtagtcgctctgaacttgcgtttctgcttacagcgtagccttagctccttacgaagacgtgccagtcggtcacgaccaacgatgatgccattctctgccagctccgtctggagccgccgggttccatatgtttcgcgagtgcggatatgtgccaccttaatctccagttttagccgctcatcactttgttttctgtctgagggttcatgctgtacccagttgtaataaccgctcctggatacaccaaatacctgacacatcgcttcaatgggaaattgttgtcgccattgttcgattaacgcgtatttttcagcgactcctgtgcaaaatacgctgttgctttttttaatatatctcgctcaaggcgagcttcatttaacgccttacgcagttgcagaatttcagattccagttcagccaccgtgcgggaaccaggagtaccgagcccttttctggcggcggtaacccattgtcctaaagtgccttcaggaagagataatcgggaagcgccttcactgatcgaaagttgattttcaggaaccgttctgacagcttcggctttgaactctttagagtaacgttgggtttttctgctcattattagctccttctgatgccattctatttcaggaaggagtgtccgttaaactcaggctacctcatymrwwwmmrrwwmmmwwmmrwrmmwwmmmrmrmrmwrrmmmmrrwwwmmmwmrmmmmmwrwrmamcgtgggcggtgatggcataggccaggtcaatatgttgctctgcccgctcctgaccggggcgaatcacacgggtctgctgaccgtcagaaagtgtgacactgtcaccggaaaccgctgtcaccgtccagacgctgttggccacataaccgcgctcccggtcactcttcgtgaagcgtatcctgtcaccggtccccacccggatggtgtccggggtgtacagcgtgacgccttcagccaccgcctcccggggcgaaatcagccgcgtgttaccttccgcatcctgcagggttatcagcccgtcatccttgctgataccggcaatccggtgatacacattatccaccagggcaagggcatccgggttattctcccaggtggagagacgacgcagctccccgtcgcgtatattcgctgtgttcaggacaggcaccatgacctgcacctgccccagttctccggccttttcccgtgcatcatgaatcatgctgttcagtacgcgccgatcctcattcaggtgcgtgacaatcagcgtctgctcccgtgcttccggtgtcctgccggtatagtcgcggacaatggcttcatacagtgtcatggggacatccggaaaagcctcgcctttcagcatcgccttctgctgcgcttctgccagtttcgcttcctggctgtgactgaactccgtcacggagtgctcmggtgcccatgcgccctccagacgtggcacctgagacggtttcacacgctcaagcccggacagcgccctttccacatcccggttaatcaggctgtataccgcctcccgcagttccggcgtctgacgcacaatctccttcatgatgaccacatcggcagcactgcgcgtctgctggagacggaaaggctgaccaggcgcgatggcctgcagctggtccgtgtcaccgctggccacagcccgaccgccaccgaccgcaatcagggcgtatgcccgtgccatgtcggtattgcccaccattgagctctcatcgagcaggaacagcgtgttgctgaaatccggcgtttctccgctgcgctgctgcagctgcgtgtcatgcagaaaggacgccagtgtctgcgcatccacgccggcgctgcgcatctccccgaccgcgcggtgcgtgggacccagcccaacgacacggggacgttcactctccggcagcatgttcacggctgacatcaccgcccggaactgtgtggtcttacccacaccggcatagccctgcaccaccgtgaaacggtcggacgtttccagtatcatacgggtggcggcgcgctgtcctgatgtcagtttctccatgagttcgccaggtactctctccatcagcggcgtgaccgcctccttaccttcgagaatatggcgaagaatgctcttttctgcctcatacgacgcacgggaaaccagcaggcctgtgccatagccttttgccacatccacatacagtaaatcaccccgttttatctgcgcattgatttcccctcccaggtcagcaaaaccggttccttctgcagcaaacgacttcgcctctgtcagcaggtccaccatgctgaaggccaggtttttactttccagcacaggaagggccagatgaatggcctgctgtgccggcgtgtgaagcccggctttctgcagactgatagcggtttccagcaatgtctcaccggcacgcgcctttatctgctcagaaaccaccgtaaaggagggatggcgggcaagtttttccgcagtacgggtttcatccagtgaggaatacagccggacatcacgaccactgcgggccagaccgttcagggtggcattgtccattgccatctgtgtgacggaggcaaaaaccgtcgcactgtcgctgacggaatgcccgggcgtttccacccagccgttctccagcttcagtgccgtgaacggtgaatcgctcacaggcagggtggccggttcagcccgtcccggcacaacaaccgtcatcgcatcttcactgacggatgccacctgcaggcggtcaccgccggagacgcggagtccgggaattttccctgtcaccctcagacgctcaccgtctgccaccggcattttttccggccggaacagcgaccagctgctgtccagggaagaaatacgcaccacctgcgtttcgccctgcgcattccgcagggtcaggctgtgactctgcgccgtcacacgttctgtcacatagcggtcatgactgcgtgtctccgggttccactgctccatcaccattcccgggcggtacatatcccgcagataacggctccggctgtccagccagaccggtgacagggcggtcatggttacctccgggtgtccgagcacgccctgtgttttcagctcactgcgaatggcctgtgtcagtatggcctgttcccgtactccgctgacctgtgccacgctctcttctccggctttcacgctggccgcaaaatctccggccagccgggcatagcggacattacggtccggttcactgatgatggtggccggtcgctgttctcccccctgccagcgatatgtgttcaccccggcatccttcatggccatcagtgcactgcctgtaccggttcgctgcccgctgtmggtwawmmggammtgtacgttatgacgtgcggcaccatccagcagggttaacgtctctttcagggagagtttttcgccctggtcgacgataacggtactgcccggcgtgaaggccatgccttccagcaactgacgacgtccggttatcagttcaccggacagccgttcatcctgcttcaggttcatctgcgagcgacggtcagcagcgataatctgcacctcccgcccctgctcccgggccatcatgaccagttcagccacccgctcacgctgcccggctgcaccgccctgcccggacacaatggccagcgacggacggtcctgtgccagaacactgacggcatcgctgtaaccggccgtccgggggacacttttctccggatgtacggtcacccggttctgtttcatgatgtcacgactgagtgcccggactgacagctcatcgagcatatgaatcccggacgtgaacagccccttctcacgatcgagggggataagctgctcacggctgatggcctcatcgataccggcgcgtgcccgttcaatcacaccattttccggcggcagtatgccaaccgtcctggccagcaggtccgtatacataaactgcactttgcgttcacttaatccggcaatggcctgtgtcaccgcctgctgcacatccggcccgtcctgtgaagcgggtccgggtgtctgcgtgcgggtatatgcccgttgttccgctgcgtcacgatatgcccggatgtcgaacccggtttccttcagcgtctgcatccactcggccattctgacttcaggatcgacgtgctgtttggatttacgcgtatccagcgccgccacatcccgggatttcagcgaggcgtcttccccgacggcctcccggatagtctgtgagcgtccggaaaaggcctccaccggtacacccggcatttcccacataccgtgcttaccgaccacttcagtttcatagcccagcgcctcaacctgctctttccgtttttcccggtagagcctgccaaaggcaatctgattagcgtacacattctcaatgaaccccgttttccccactttgtcactgctcagtgtcttccattcgccgttatgctgcgtaacattagtcaccaccgcatgcgtgtgtaactgtggttcctgatcgcgactggtgtcgtggttaaacagtgccatcaccagattaccggtcagcaccgtttctgactgtccgtccgtcatcacccgtgtggaggccagtgcctccacctgacggacagcaaaatccacggcctggttatgtgcttcaataagacgtttgtcccccccgagcatggccatcatggagacacttttgggagcggagaaggtcaggtcgtagccgggacgatgcctgttactgccatcctgcatgcggcttaaatccgccccgtccggcagcctgccctccagaaggcgggtaaagacatccttatcgacactgccctgcagccccagttgttcagccccctggccggcccagcgttctcccatgctgcccagcacatagtaattatccttgtcggtataataattatcggcacttccggccgatctgacctgcgcgatactcatcataaacgtatatccctttgacatatcccggcatcaatcccacaatagatatacacaagacatatccacataaggaggcaaataatggaaaccaccgtatttctcagcaaccgcagcctggcggtcagactgccaaaagcggttgcattgccggaaaacgtaaaacgcgttgaagtgattgctgtcggacgaaccagaatcattacgccagccggagagacatgggacgaatggttcgacggacacagcgtcagcgccgattttatggataacagggaacagcccggcatgcaggaaagggagtcattctgatgctgaagtttatgctcgataccaacatctgcatttttacgataaagaacaaacccgccagcgtcagggagcgttttaacctgaaccaggggagaatgtgtatcagttcggtcaccctgatggagctgatatatggtgcagaaaaaagccagatgcctgaacgtaatctcgctgtgatcgagggatttgtttcccgcattgatgttctggattacgacgctgctgcagccacacacaccggccagataagagcagaacttgcccgtcagggacgccctgtcgggccatttgatcaaatgatcgcaggtcatgcccgcagtcgggggctgattattgtgactaataacacccgggaatttgaacgtgtgggcggcctgagaactgaagactggagctgacctgttcatcagaaatcatctcccggctcaacatcctccccgcgttcccggtgcacattgatgttcacctcttcacgacgctgcatctgctgccaggtgtccggatgattttcctgctgccatgcctcataagcggccatatccaccacttcaccggattcactgatcccgggtggcagttgctgttccatctcttcttccggtttcattttcagctcctgctcaatcccccctgccggaacattcacacctgaatctgacttcttatcgttgatgacaggagacaccggctgttgcggctgttgcggctgttgcggctgttgcggctgttgcggctgttgcggctgttgcggctgttgcggctgttgcggctgttgcggctgttgcggctgttgcggctgttgcggctgttcagcctgagtcacgtcttctccggaaacaacctccgggacatccggttcgaagaggctggccatctgacgaccttctgcttcccttgcggcaagtacggcactcagacggttctccatttccgggttgatgtcacgcggaataaactccggagcgaccttcggacgttcctgatatttcagtgagagtttgactgccggatacggtccgggcagggtgacatagcaggtcagatccggcagggactgaatgtcggaataactgaccagcgtctggcgctccatatccttaccggtcgataccccgtcacgtaccggatcagcaccgtaggaatactgttcgctggctttcaggtgctctttctcaccaatttcaccgacagcgaactccgcaatcttatggctgggagaacggaaaaaggcacgggtgttcatgacgtcaaacagcgtggcagccgctttctcaccgtagatatcttccagctgggcataggactggataccaaacacataacagccaccgaacttacgggcttccggcaggatctccaccaggtccggcagtttgtgtaatgtgggtaactcatcacagaagaaccacacacgacgattacggttttctcccattgccagcagaccacgaatggcaatggacagccacatggagatcaccggcttcagggaggcatgagtgtcggcattcgacgagataaacagccagccgtttttctgatcctcccggacaccacgcatccagtcacggatggtgaagggctcaccgttatgctcaatcccctgcaggtaacggatggctttcacatagttggtcagcacagcacggatggaaatcgccgttttctcaattttctcttccaccaggttggccgccggtgaattacgcaggtaggtacgcagtttttcaattttgatggaaagcagtgtgtccaccagtttgctgtagctgcggttggggtcattacgcatcagatacgccgcttccgcaaaaatggtacgtcctgaaccctgccagaacgggtcctcttttgtgcccatcgggatcagagtatttgcggtattatcaaaatccggctgtgtcaggcactccttccacaaatcccaggcggcacagcgtgcatccagcggattcaggatcttatcgatggagggatcatagtaacttttaacaaattcccctgaacggtcataaatcaccaccatatcaccacgctgacgggcgtagttggccagacgacggataacctccgacttaccggccccgaccgtgccgtgcagacagaagttctggatttcggaatcccggataatcggcagatcgccaatccggatatcggagtccctgccgtcttttttcagcatccgggcaacgtctttcggattgtctgtcagttgacgaccacctgtgacttcattttcgctctgttgtttaccctgacgccccagaatccaggagacaacaaagaaggtaatcaggcaaataaccagtgccacaactgtggccagaacgaatgcagaccataactgctcaccgcaccagatcatatatttatcatgcagtacctgagcagcgttcatccggaaggttttgccgtaatactgaatctcatataccggctgagactwgawwmmmtmmmgmatgcmttmmmgcgtggtacacmaccagtmmmtacagccattcacaaacgtctgccagcttatttttacccmwmmmmmmmrmmmmmmrmrwmwmmmrmmmmmmmwmmmmmrrmrrwmmmrmmwmmwmwwrrsrmwmwrrmwrmmmmwrmrrmwmmrmrrwmmwrrmwmmmmmtwattgatagtgttttatgttcagataatgcccgatgactttgtcatgcagctccaccgattttgagaacgacagcgacttccgtcccagccgtgccaggtgctgcctcagattcaggttatgccgctmaatgaggtgtactggcaatagcggacactaccatttgttctttttttaagcagccatctgatgatatttttccctgaaggctgccggggagatattccccagacgagagtgacgacgctgacgattgtagaaaatctcaatgtattcccgtattactgagatggcttcatcccggttattaaaacgatagtggctcaggctctcatttttcagcgttccccagaagctttccatcggagcgttgtcgtaacagttacctttacgcgacattgatgttttcagaccagactgctcctgtatgacccggtaatcgtatgcgcagtactgtgaacctcgatcagagtggtggattagcccggcaggtgggcgctggctcctgagcgccataaacagggctttacctgtcagctcttttgtcatgcgctctcccatggcgtagccgacaatttcgcacgtataaacatctttgatgccagcgaggtacaaccatccctcctgtgtggcaacatacgtcaggtccgccacccagacctgatttggtgctgtaggagcgaacgtctggttcagcagatttggcgcaactggcagattgtggttcgagttcgtagtcgctctgaacttgcgtttctgcttacagcgtagccttagctccttacgaagacgtgccagtcggtcacgaccaacgatgatgccattctctgccagctccgtctggagccgccgggttccatatgtttcgcgagtgcggatatgtgccaccttaatctccagttttagccgctcatcactttgttttctgtctgagggttcatgctgtacccagttgtaataaccgctcctggatacaccaaatacctgacacatcgcttcaatgggaaattgttgtcgccattgttcgattaacgcgtatttttcagcgactcctgtgcaaaatacgctgttgctttttttaatatatctcgctcaaggcgagcttcatttaacgccttacgcagttgcagaatttcagattccagttcagccaccgtgcgggaaccaggagtaccgagcccttttctggcggcggtaacccattgtcctaaagtgccttcaggaagagataatcgggaagcgccttcactgatcgaaagttgattttcaggaaccgttctgacagcttcggctttgaactctttagagtaacgttgggtttttctgctcattattagctccttctgatgccattctatttcaggaaggagtgtccgttaaactcaggctacctcagggtcagtmmwrmmwmmmmrmwrwmrwrrwmmmrymmmmmmwrrmrrwwmmwmmmmmrrgargtmagrmwrmwrmmwrmrrmmwmmrmrmwmmmsrwmmrrwmmmmmmmrwwwrmmmmrwwmmymmwrwrrmrmmrrwmrmmmwsmrrmmwmmwrwmammgmmammrtmmrrmmrrmrrawrmmrtrmmmrmmkmmgccytsatattccggcatcattttwtkcgccagcrmrtmyacmkacgcywcrgcawmmmgammatcswkrmmmmmmrrrwmmrrymrmmmrrmsmrmmrrwmrmmmwmwwmmmmrwwmmrmrwrmmmrrwwwmrmmwmwwrmmwrmwrkwwwcmtmcgttgtwmmmmwrwwmyrmrmmammrrrmrrtgccgtgcctccatcttwgccgggcswtaaggcrgggatgcacarrrcrcmmrrrmmrmmrmmrmmyrmmrmrmmmkrmyrrmrrrarwwrrmrrrwrmrmmmrmrmmmwmkrmrmrrrwrmmmkmmmrrmmmmtcacrrcagacrrgaargarrgaccgggtgaccgcagcggcgaagcgtacccttgcatcaacgccagcccggctamgatggaggcacggcacarmmmgcammcgrcgctgaggcyacakarctgaaaacccacaaggccatgccccgcatggccgggtaaagcggagcckgcggcgacwtcctttgtgctctktccggggaccggcwggaaaaaggcggggatttaccccataagcgctaacttaagggttgaaccatctgaagaacgcgacgcctcggtgcctcgttaagacgatgcctcgcgttcttcaattgcgttttgtaggctgtcagggatactgtcccacgaatggccacctgtaagctccagatgaccatttttgttattctccacaacgcgttagttcttcttttcggatccggcacttctggggggaaaatccagcgatggctggattatgtcgtcaattaaaaatgcggcgagtagattagcaaatatccacgctttcgcgagttcaagttcctttgcacgcaaagcatccaagtgcagcaaacttttgagccgcttaaaagccagttcaatttgccatcgcagacggtaacaatcagccacttgctctgctgaatattcatcttccgataatgatgttagcaatagcacatggcccgctgcttccagcgtttccgcctgaactactcgtccttttcgacgattctcgctgagcagtcgggttttactgattaatgctttttcgggaggaagtgatacggcaatgagacgtgcaggaaagggagctccggcttttttattacctgaattgcctatcattacagtggtttcaccgttcttaccgcaatccagcccgcgcagaaaatccatcatgtcaaagcgcattccttctgcagttaaccagcgcaatcctcgccagtaaacccggacgatataatcagcttctccaaaagcaagtgagcggatacattcgggacgcgaaccgaatccccggtcagcaattcgtatctcgtctgccgtttgcgcaaatcgatccagccgttcagcgtctctgctgtcggttagctcaaaatcagtgaactgacaggtatgaggatcatatcccatatgtagtcgccattcagcgctgccgcccccgggcgcgctgattgctgttccatcgacaagacgcaatcaaggtgcggcgggagtcgatggcatgcgcgtaagtgaactgccggactacctgaggcaccactggccagagctgaaagcgcaactgctgtccggcagataccgcccatcccctgtgagaagagtatccatactgaagcccggcggcggcgaacgtctgctgggcatccagatggtggtggatcgcttcatccagcaggctatgatgcaggtattgcaggcactgtgagatagctcgttcagcgacaacagctacgggttccgtcccggtcgttcggcccaccaggcagtgatacaggcccaggaatatatcggggccgggtatcactgggtggtcgacctcgatctggagaagttcttcgaccggataaatcacgacgtgctgatgagccggatagagaaacgagtatcggataaacgggtgttgtcacttatccgcaggttcctgaatgcaggggtaatggacgcaggtctggtaaggccggtgacagaaggaacgccgcagggcggcgtgatctcgcccctgctctcaaatctgttcctgcactatgcctttgatatgtggatgcaacgtcagtgtcccgacgttcccttcgaaaggtacgccgatgatggtgtgccacgaaggcgcgcaccagggacgtgagtccctgtgcagccatgctgcacaagagatgagggaaggcccctcggtgtcggcgtgcaggcgcaggcaccaaaccacccactgctgctgcatttaagagatgtggtggtgagcgagatgggaaaaggtcggacgtgagccgatcaggtatggatcgaagagacgaacgaaagtgaaccgctgatgatgtgtcgaaagcatatagatgatgtcaaaaccatggttatccggctccggcgttaacgtcacgggaagcagtgccggcatattttttccggaaggcagcaggccacctttccggtattgatggcgccagttgaagagcaggttatcgttgattccattttcacgggcaatctgcgccacacaggctccgggctgcagtgactgctccactaaggcgattttgaactcataagggaagttggggcgccggggacgcttttttaccccggggacttcgggcataacggtactttcagtaagtacgacaggtactgtggaggcactcccgtaaagacgggcgtcaagttcccgctccgacatacctgcgggcagaggccatgaaaggccagctttgcggaagcgcacaaacatactacaaactgttgattttggtacgcccaggcgacgtccggctacaacgaggggtaaatgttcttcaaagtgaaggcgtaaagtgtggatggtgcagaagatgccgatttccagcccccgttttcgggctgcatacagcagatactccaccgccagacgacacacgtcattgcagccgtcaagctgccgtgrttcmgtgccgttctaaatagctgcgcggaatagtagatcactttgagggaacttagcccggattgtgcgatctgatcaatcgccaaatcaaaacaaatcaccaaccggactgagcaatgccgatcatagcaccaatttcccgtgacgaacgacgcctgatgcagaaagccatccataaaacacacgataaaaattatgcccgcagactgactgccatgctgatgctgcaccggggcgaccgtgtcagcgacgttgccagaacgctctgctgcgcccgctcctctgttggacgctggattaactggttcacgcagtcgggtgttgagggactgaaatcattacctgccgggcgtgcccgtcgctggccgtttgagcatatctgcacactgttacgtgagctggtaaaacattctcccggcgactttggctaccagcgttcacgctggagtacagaactgctggcaataaaaatcaatgagataaccggttgccagttaaatgccggaaccgttcgccgctggttgccgtctgcggggattgtgtggcgaagggctgcgccaactctgcgtatccgtgacccgcataaagatgaaaagatggcagcaatccataaagcactggacgaatgcagcgcagaacatcaggtcttttatgaagatgaagtggatatccatcttaatcccaaaatcggtgcggactggcaactgcgcggacagcaaaaacgggtggtcacgccgggacagaatgaaaaatattatctggccggagcgctgcacagcgggacaggtaaagtcagctgtgtgggcggcaacagcaaaagttcggcgctgttcatcagcctgctgaagcggcttaaagcgacataccgtcgggcgaaaaccatcacgctgatcgtggacaactacattatccacaaaagccgggaaacacagagctggctgaaggagaacccgaagttcagggtcatttatcagccggtttactcgccatgggtgaatcatgttgaacggctatggcaggcacttcacgacacaataacgcgtaatcatcagtgcagctcaatgtggcaactgttgaaaaaagttcgccattttatggaaaccgtcagcccattccccggaggcaaacatggactggcaaaagtgtagcggtattaagcgcagctatttaggcataaattaaatttaacgaactgcatctacacagtcctccgtgccaaacgggcaacacacggaagggggatgtgtttcgattggtaacctgtaagaaacaactagtggaaatacctgtactttataatttcaagggtacgggtccgcttcgtttgatttgcgcgtttttaacaattttaaaacccagatatactgttccggatgtgaccccaccaaaatttccactgttttgttcatttgtcgggcaatcattttattgtccgcgctggcaatgcatgcatccattggtggccgaacttcaatagtcaggaaatgttttttttcatcataaaccgggaaaagcggtataatcatagcctgactgatgttcatcagacgtccaataattggtaatgtcgctttataggtcgcaaaaaaatcagcaaattcactgtattcaggaccatgatcctgatctggaaggtaatacccccagtatccctggcgtactgactgaataaatggttttatcccatcctcccgggaatgtaagcgccccccgaatttacgccgtacagaattccagacataatcaatcactggatttcgttgctgatgaaacatggcggccattttttccccctgagctgccagcaacattgcaggaatatccacgctccagccatggggaaccagaaaaatcacatttctgttattgtgccggatttcctcaagaatttcgaggcctttccagaacactcgtttctggaacttttccggaccacgaattgccagttctgccatcatcactatggattgcaatgctgtggcaaacatattgtcgacaattatttcccgttctgtatcactcttttccgggaaacaaagcgataaattaatcgtcgccctgcgacgagcttttctgctcaatctgcccacccaacgccctattttggccaagacaggatctctgaatgaagggggaaacattgaaatacctgcaatcattcccaaaacgaaccatgtaaaccagtaaacaggggaaagataatttttcttaaattcaggaataaactcggatttgtattttttcattatttagtgcgaatgcccctaccaaaatcatcattaatattttattcagagaatacatcaaaccttaatttaagtcctgatgttctgagtttacaggatatgcatccaaagagcggatagcagtttcatggatataatccaacaaagcgtatcgttttcgatactcagagcgttttttactagctatattgcttagcggctttcgaaccacctgtgttggtaatttgtaccagtcaccacaggttttccctgctacagactcccagaaatcactgtagacagcaacaaaggttttgcgtttctgataccagtatctcaattgccggaaaacatgactgtgctcactgactgccagacactcagagacattacaagctttcatcaacgcacaaaatgcttcaaaaataatccgttttgggaaaagcccataacaggcccgggttgcgcaccgaataacatcaggtccggtatttttgggggctccctgtaatccgccaataaaagcggtgtgtccattttgagtcagaatgactgaaaatgttaaccgggcaatcactattttattgaaaaacaatgacaacatcaactctccttcccggtcaaaaccactgggagaacactggatatcaaaatcctctccatctttcccgatgaattggactaatgataccgatttttgtggaaacaaaaattcagaatatttttctggcaaattttgcacaaagcgataatgctcaagaatatattgcccccgggaccaggctcgcccccctttatgcaaataaggtcgatgaattcttgctggcaacgtgggttgtattgccagaatatcctcaatgcactgcaaggaacgcagttcgtaataatatcgaatactggcaaccggatttatagcacatcgcaaaatgtacatgaaccggaattttctgtgttcccacttccccgggagtgaatctgaggaaaaaacaatccttttaaggaaaccgataaatgataagttacttacagaaaacatattgttacttgtgcttcgctaatgtgagtatttttaacgcagttttattaatatcaaaccgctcttctatatcctgttttgcctggcgagccatctgataaaaacgcccagggtcattaaaaaaatcattcagggcgcacatccatgcgtgcttattacctgctggtaacaatgtcccggtctggttgttctgaataacatcagggattccaccaatctgactggccagtacaggcacagaaaatgccgatgcttctgccagtaccataccaaacgattcgttttctgaaggcagaaccaccagactggcaacccgatatacgggggcggcaggaaaaacattgtcagcaataaaaacatcatcatgcatgcccatactgtcaatctgatactgtaaatgctcccgcagttcaggcgaaccagaccccacaatcaaccaacagaactgtcgtccattcatttttaaatggaacagtaaattcaacataaattcgtgccctttttccgggcgcaacattcctacctgagcaataacggggcatccccttcgggacgccagccaagaaagaacatttggaggcaaactgtttcgggaatcaacgtataatttctggaaatcaaagcctggaggcacaacagtaacccgggttcgacatccttcctgctccagatgtgtcttcatacttgttccgggaacaatcacctcatcgcaaaaatgatttattgaaaaaacttttgtttttcgtgtcaaatatgtcttttgcctgataattctgaatggatgtttccaagtgaataaccgtactaaaccaacaatattgctatcatgcccactgtgacaaacgattgcattgggctgaaaaccatgaactattccgagtaatctccatgcagtagggatgtgaagactgtttcgaaataacgcgaatgtgatatcaattcccaatttactggcttcaaaagcaattttgctgttttccctgcagacaagcagaactgaatgccccattttcttcagggccttcatttgagcaacagcctgtaattcctgaccacctatattcggtgatgattccgtaaatagtatattcattattaatcatccggtaatcttggccccttatgaaaggccagcaacgatgaaaaaaacggtgttgtataataaaaaagtcgtcttttcagccatgcgcaactttcccgttctttggtgcttattcttcctatccgtgtagttcctttagcaggagcattcattcttctttctgttgtatataaataatagaatccaagttcttcagcgacctgaatataatccttgttataataaccttccggccaacacaaatgtttactgcattttcctgtcatttctttcaaatattctctcccgctcaacaagtcctgccgaagatgtttacactgttcttctcgtgaactgaactttttatcccagcgtgtatgagtatgcgtatgaacatgaaattcgaccagaccagattgcaacatttcgtttacttcagaccagcgaagcataacattatcagcattccctgtcgctatttgatgttcgcaatcgcgatgagaatattcctttccaggagagtgacgaaccggaccattcccaataaaaccggtaattaaaaaaatatgtgctttgagattaaactcttttaataaaggatatacctgaaaccagttgtcaaggtatccatcatcaaatgtcaacataacgcttttccgggggagttttccgccccgataaaaaaactccagttcatctgatgacagtgttttccagttgttttcagccaaccatttcatttgcttccggaatgtcacgggagaaagtgtaaccagtccggggcaatgactgacatgatgatacattaataccggaacatgattggctttgaacaaaatgcccccttcgtttaacatttaaagtttctggcacctagtaagcagagacaaaaggaaaacccaataaagctggtgcattttatttccagatactggatgaataacatagcaaacccatggcatatttgatataatttactaaattaaacatcaacaattcataaacaatgatatattatcatatcttttaaaaacgactcactaagaaccttgaggtggcacactgaatttggctacctgaacagtgaggtgtactggcaatagcggacactaccatttgttctttttttaagcagccatctgatgatatttttccctgaaggctgccggggagatattccccagacgagagtgacgacgctgacgattgtagaaaatctcaatgtattcccgtattactgagatggcttcatcccggttattaaaacgatagtggctcaggctctcatttttcagcgttccccagaagctttccatcggagcgttgtcgtaacagttacctttacgcgacattgatgtttwmrrmmmrrwmyrmwycwstatgacccggtaatcgtatgcgcagtactgtgaacctcgatcagagtggtggattagcccggcaggtgggcgctggctcctgagcgccataaacagggctttacctgtcagctcttttgtcatgcgctctcccatggcgtagccgacaatttcgcacgtataaacatctttgatgccagcgaggtacaaccatccctcctgtgtggcaacatacgtcaggtccgccacccagacctgatttggtgctgtaggagcgaacgtctggttcagcagatttggcgcaactggcagattgtggttcgagttcgtagtcgctctgaacttgcgtttctgcttacagcgtagccttagctccttacgaagacgtgccagtcggtcacgaccaacgatgatgccattctctgccagctccgtctggagccgccgggttccatatgtttcgcgagtgcggatatgtgccaccttaatctccagttttagccgctcatcactttgttttctgtctgagggttcatgctgtacccagttgtaataaccgctcctggatacaccaaatacctgacacatcgcttcaatgggaaattgttgtcgccattgttcgattaacgcgtatttttcagcgactcctgtgcaaaatacgctgttgctttttttaatatatctcgctcaaggcgagcttcatttaacgccttacgcagttgcagaatttcagattccagttcagccaccgtgcgggaaccaggagtaccgagcccttttctggcggcggtaacccattgtcctaaagtgccttcaggaagagataatcgggaagcgccttcactgatcgaaagttgattttcaggaaccgttctgacagcttcggctttgaactctttagagtaacgttgggtttttctgctcattattagctccttctgatgccattctatttcaggaaggagtgtccgttaaactcaggctacctcacakmrrwrmymwsywmrmmwmmkwmmmmsmmmkskrmwmmmmwrmmmmmmmrrmmwwwymrmrmmrmrwwwmtttacggggtgacgctttcattaacctgtacacgaacttctattcckgcatcrtgacaggcckgcagcyactgcgccayttcmagmrgatmgcmmtmmmggcgtacmaccmmrccttctttattccataactgcagagcatttttctgattgttccatctgatttcggggggtattccgggcaaggttgttcagtaaggcattccttcgtaaaatcagcatgttttttgcacgacggaaaccggcagccatgaaaatctgtctttatcatactctgaatcctgacacgatcccgggttataaaaaatttgcacaggctattgccaccgataactttgttcaggctgatgttcgtaaaattgacaccaatctttaccgcgcccggctcagtatccgggaccgattactgttttcgctgtatcgctaccacggtgaaactatctgccttgttctggaatatattcgcaatcatgcctataacacttcacgttttcttcgcagaaacgtggtgattgatgaagggcgcttacaacaacaaccagtgccggatcctgttgatatcgcgactgaagcgctgacgtatatcaatccgtctcatgggcgtttccaccgcctggataaaatgctgtcctttgatgatgatcagcaggcactgtacgaacacccgctaccgctggtgattgttggctcggcgggaagtggtaagacggcgctggtactggaaaaaatgaagcaggctgctggtgatatactgtatctcagcctttcctcatttctggtggaaaaagcgcgtactctctatgacgcttcaggggaagggagtgaggtacagaatattgactttctttcgctgacggaatttctggaaactctgcgcattccgaaaggcagggaggtgactttcagtgcgttcagcgactggctgccccgcaacagggctatcgccgcactgggggctgcccataccctgtatgaggagttcaggggcgtgatcggtgcggttgcctccgggaatgggccgctgtcccgggaggcgtatctgtcactggggatacggcagtcgctctatggcatggaggaccgcccgacggtctatgtcctgtttgaacgttatatcgcatggctgaaacagtcgcatcagtatgacagtaatctgctcagtcatcagtatctttcgttggcaactccccgttatgatgtcatttttgttgatgaagttcaggatatgacgcctgttcaactgcaactggtactgaaaacgctacgtcatcccggtcagtttttactctgtggtgatgccaatcagatagttcatccgagctttttctcatggagttcgctcaaaagtctctttttccggcagcaacagggaaacgacacaaccgttaatatcctgcaggcgaattaccgtaacgggcaccacgtcactgcactggctaaccggttgctgagactcaaacaggtccgctttagtgctattgatcgggaaagtcaccattttgtcagaagctgcggtcaggcagaagggaccatccggttgctggacgacagggaagagacaaagcaggaactgaatgccaaaacctcactttcgaatcgtgtagcggtcattgtgatgcatccggaacagaaagcacaggcgcgatgctggttcagtacgccgctggtcttttcggttcaggaggttaaggggctggaatacgaaacggtgatcctttacaatatagtaagcgctgcccggcaggcatttgatgatatttgcgagggtcttacaccggctgatcttgagggcgaggcccgttattcccgtccgcgggacaggcaggatcgttcagcggaaatctataaattttttaccaacgcgctgtatgttgcgctcacccgtgctacacacaatgtgtatcttgttgaacagcaggtggagcatcctctgtggagtctgcttgcgctcacgcatcaggaagagcctctgaatctgcaggaagagatttccagccgtgatgagtggcagaaaaccgcacatctgcttgaaaaacaggggaagcaggagcaggcggacaccatccgtagccggatcctgcagacatctgagatgccatggcagattatcaccgcagaggatgcacgacagtggaaacagcatatcctcgcaggtacagccgataaaaccatacaactgcaggcgctggagtacagtctgatttattccctgttcccgctgtataatgcactgtaccgggaagattttaagcccacaaggcagcccagaacgaagactctgcagttactggagctgaagtatttccggccatactcaatgaataacccggttgccgtgttgcgggatattgaacgttatggtgttgatcaccgttcgccgtttaatcttactccgctgatgtcagcagcccgggcaggtaatattgcactggttcagttactgctggagcgtggtgcagatcccttgctgaccggtaatgacgggctggcggcttatcatcaagttctgtcagcggcggtcagtacgccccgttatgcgcaacagaaaagtgcgcagttgtataccctgctgaaacctgaaagtctgtcactgcaggtggaaggacgcctcatcaaactggacaaccggcagatggcaatgtttctggtgatcctgatgcaggcgctgttccacacgcatctgggaagtgctctgtttttttcggaagccttcagtgcggcacgactggcagagtgtgtggttcatcttcctgaagcgttgctgccggaacgccgcaaacgaaggagttatatcagcagccagctctctcaacatgaagttaacagcaaaaatccttatggaaaaaagctttttctgaggctgaaccacggacagtatattcttaacccggggcttaaaataaggcagggtgatgtgtggcgcgcggtttatgagttgcagtcaccggaagatctggggcatgatttgcagacttatctgcaggatatgtcaccggaattagtggatatgctgggagggaagaagggtttttacgaacgcagtgaaaagtctgtcggatactgggtgggggggatccgtcgtgccgctcaaaaagcgtgaggttctgtgctgacaggatgtaaccgaacttcatctgtacgtaagtacgtgatgtctcccgcccacttctggttcggaccactggcgtaaaaatcctgctccagcagattttctgacacgggcaggccatgtgcgcggtaactgaccgggctgaacttccgggaggcctttgccctcagcccctgacggcgcaggcttgccgccacggtttttacgttaaagtggtaaccctgagcacgcagttcatccgtcaggcgtggtgcaccgcaacgctgttttgaccgggtaaagaacgctgtcgcagtgctgacggaactgttgacgagggcttatccctgtccgccgctgacaccacgttcaacagtttgtggactgttcgctcgcttccggaaatcagggctttcatggcctttgcctgcaggtatgtcggagcaggaacttgatgcccttctttacggaagtgcctccacagtacctgtcgtacttactgaaagcaccgttatgcccaaactcccggtggtaaaaaaacgtccccggcggccttaatgccagtcagttaagcaactgactggctctttttcggggctgtggggtatttccagggcctctcctttaccactctcgggaaggccctttcccttctcgtcggtaatttcacaagttgtcccatacttgcaagatcgcgcatcagctccggtatacgtcccggtgaagcgccctgcaatgtcatcagcattctcatcaccattccgcatgattctgagaaactcagttgattcggccagtaacctttcagatgttccgccattttaatcatctgatatctcaccagattataagccagtaagacaccccacagctcttgctccacaagctccggctttttacttctcagcgtcagcctgctcagttgcatcgtctgttttatctccctgtatcccagttcgatttcccagcgatgactgtacagatccgccatttctcctccggggaagcgcatggcgtccgtcatcgacgtcagcagawrrcmgmctykwmmwywgmgmgtcryrrtmmkyrrkmrkrykkymrywwmmwwwmymmrwmmmkkmyamwwykwwmstssmygcrrrmtrkwwwwmrrmkwymmyrrmwrmwmrmmywymmmymrwwyymkrmrswmywmmwmwwryrywmmywykmkrrkmkkwmkyrwmmrrwrkmskkkywykymysmmmrrmwmmmsrymwwwwmsmrwsmmmryrmrwmrymmsmwkwrkmmmywrmmrwmmrrkwkywrymrmmkrywyswwmymwmmsyyrmkmmkmrmrmkmrkywwmkmwswwmykmmyyrykyyrrrsrmwsmrkmmskmmryrrmwrkmyrryymkywssmymysrmmrmmsmkwywrmmmwkmrrrwmsrkmrmmrkskwmmmrrmmyryrkmyrrmrrrrrrmksmysmryyrwwswmykrkrymymmrrkryryrmmmrmrmmymmmrymrrkkrmymkmwskskmrskmmsmmmymskksrkmkkyrkmkkkrsskymwkmmmsrkswrykmkkywwyyswkrmymsrmkksrrmymrmmymmmkyymsmrrmrswrsyrrrsmyrmmwmrmrrsrmkkrsrksrmmrmmsskrmrrkksyymkssrkmrwrmyrkymmrkskrwymrmrmkmyrkykmmkmrrywmywwmmrmymrrrmrmymykmymmmrrkrymyyrkrmsrkmmywkmrrrskrrwrmysrmrmkkkmrwrkyrkwmsmswwmmykmyymrsymrrsmmmmrrkmrmysmswymrrkrkmkwkswrmyyymmmrrrrmmrkswrkkrkwwmykmrkrkmwwykyrmsrrrmyrymrkwwmrmkrrmykrymymmyryrmrkrwmmmmwysmkrrmmmmmrkrmkskkkwmyrsrykykwmsmsrrmmwywkrrymkskmmmmwrwkmmwymrmwrmwmrrmmwwmskmmgccggggacgtttttttaccaccgggagtttgggcataacggtgctttcagtaagtacgacaggtactgtggaggcacttccgtaaagaagggcatcaagttcctgctccgacatacctgcgggcagaggccatgaaaggccagcgttgcggaagcgcacgaacataccacagacagttgattttggtacgcccaggcgacgtccggccacaacccgtggtaaatgttcttcgaaatgaaggcgtaaagcttcagtgattcaggtccggtattccatacgatagtgtccacttcaaaatgatggacattatttttgtggaaccggaggtaacaaaccagacggtttaaatgagccggttacgtttcaggtgtctctggctggaaatccgtgcgtgattcgccttgctgcacaggtatgcatgtggttaaaatttatcatacgcgatcgcggcggtttttcgggtggtttgttgctatttttacctgtctgctgccgtgatcgcactgaacgcattctggcggtgcatacaattaagatattacgttaatatcgcgagggaatggtccccatattctcatagaagagcctctggcttatagcatcaacaaatacggctataaatatttcctgtttaataacgaataaaaatcatttgtacgcatttgtaatcgtcggtcgataatctctttatcttttttggttgcagcgtaatattcagcataaatattgcgaaaatctattgggaataaactttcactttctttatcatacagcatattacctgtattaagatcgtaatggattatactcaactcattcaatttacatatcaactgtagataaaggctctctatattatcagggatatcagctgtatcaatgtcactaaggggagtcccaggcacgcgcagcattcggaggtacacatctccgccatactgtataacaacggatgcttcatcgccataaaaagcattaaaaagctcagattcttgccaagccatttccagaatctcattgtactggttgccaataagatcatactttttatacaaagcagatgaatcattcacatcttcaaagatttctgctgtcgacccctgacctattaacttaccgagaatgggctccgttactatgccagcatgagaattattattctcaaatggaaaaggtgtttgaataatggtagatgttattttcatgtcatacctttgttacgatatttaaaaatcacaagagattttcacacaaaataacgttacacgctaccttgtgtggtccagatgttccttccacacgaccgttgcggcctttatacggctgccctgccaggctgctggctgaataaccaatccgttcgtccggttcaatggcggccatcaggttctgctccgcaatgtactacgaatgcgcttcctgtcctggatggccagcatggtccagaacatmtccatcatttatcagcaatcttcctgctcatttcatcccgccatgcagcagcagaggattcgaccatgtcatggggaagcacatcaccgcattcgagttgctgacggcctgccgcaacctggtcacggaaccaagaattgtgctgccgttcaacggtctggcgcataaaatcccggattaactgagagccattacggtccatgctttttgcagcggccataaaagcatccttcagttctgcgtcgattcttacactcatattaacctgtgccatttgccacctcgtcattaccacgtatatacacagtatataactgttcagatatcgtcaccaggatatgccgcgccagcggcatgtaaggcggcactccgttgttgcatatggcagaggagtaaaaccgccgaagcccggcgtaagccggtacgtattgacaggtttcacctgtcctatcactggcgctgacagtwwwmwrmrmwrwrmwwmwrmmwrrwrmmrwwrwrmrmmrwwwwmmrrmmmwmrmmrrrmmrrwmsmwmgtcagtcagctgacggtaaatctgctgttcagtctcacgcatcacctgtgcaccggcttccctctccgcatccgcatcaccgctcagacctgatgctttcagccggtcagccaccctctgagrgtactcattctccagcatctcatayttctgctcttctgccwgcgcccagcggtcagcttccgtacgcttcagtacagcatgccatggtccccagagggagaaccagtccgtaaattcattctcttcacggcttctgaccatggcttcggcagtgcggaggtcatttgctgtcactcccgacacgccatagaaacgcatttccttcacggcagtggagagctgaagtttctctgcgagcatggtctggaaggccaggtagacttctatctcatccacaaaatggagagttctgactttatcccgggcaatgtcctccagaatttcgaggcggaacatttccctgcccagggagagcagagcgccggtatcattatcgaaaaggccttcagatgcctgatggaccaggagggttttccggagattgttccatgtgagcgcgacacggtcctcacagctctcagtggcatcagcagcaacagcgaaagactgctgtcgaagctccgcagaggcactgagtttttccagccatgcagcgacctgttcacggaatccggaggtattgcgtgcagagacggtatcggaaaggcggtcaaggaacgcggaaaaggtgttggcrtgctcttcatgttcaaaagcatgccatatctgtgatacatcagattgtttgttttccgggaaccatgctgtcacggcatcagccagggggcgatggarwrwmwwmwrwyrwmyrwmmmwmmwrrmrmmrkmmmwmyrysrrmmrwrrwmrwmmrryrmmrmrrwwmmwmywwrymrrrmwwrmmrmrmmwrrrmwgmymmwrrrwwmkmmmwmmwmwswmwwrmmmmwwmmwwcmtcarmtwmmrmmwrmwttccgggatatgacttatctgatttctatcaaaaaaatactctctgacaacgggattatttccctcagaaaatgctggtagctcagtgaggaaattacgggtcgctctgagatttttaagagcctgtgggagcgatggtaaggagactatttcgttatagctgatattcatagtcaataacgaatcgggtaaagctggcaggttttccagctcattgtgttgtacctgaagtgactccagcgcaggaggtagcacgggcaattctgataatttattgctggcggaatataatttttttagcgtaacaggcagttcaggtaggttagttaattgattatatgatacattgagcagcgttatctgagctggtaagttgtcaggtagcgaggacagatttaaacggtccagtcgaagttcatcggaattattgatragacattctttaagtmgggagacagcctcatcacgctcttcaccggggagcgcttgtttttcccatttatcccatgctgaaaagtaatcagcatatgtaccggaaatagtgttataaaaagaattttgggrcaatgaaaagttattatttatcggtaacatgatggrcatycagttaaagccgrtggacaaatggctttgttaaaagaaataagtaggagtttttatgaagttcattccatgaaatgaacttcagcacttttttattataataaggayaaacaaaattggctgtaaaaaaacgatactgtttcagttaacygtgaatggcctgtttgcaccgtatgtgacagtaatacgctgcttcatgataagccctcaaaaacatacttatgatatgtatatgagtatatattaatggtacaaaccatcaatatccataaaaaatacaccattaattcgctataagtttttaatacaattttttctgctgcgatttttttatacgataattagcatacttataacaccgtaagtaaattacatgaaagcaggcacagcgtgatccgatttttctgcgtataacgattttccggaagtgtaatacggttgcgccccgggccgggcccccttttctgttgttgtgtacgggcccacagagaatggtaccggaggcgggctcagaagtactgagtcgggtgatcgcgtcaggccgcgaagtactgacacgggtattggtgactggtgatgaagtactgccccgggtatgatttcaggctgattcaggttctgattctctgtcagtccgcgtggttgctggcctggctgatttcctgttgtcaggcgttcaacaaccgactgctggttatagcccggttcacgaacacctgacggcaggtatcactcacccggtgaaggtaaatccctcaagaggcagcaactctgccagtcgctcctccggccattccggcagacgcgtcaggacgtccgtcagccaggcatgtggctccagaccattacgttttgctgtttacagaaggctcattatctgtgccgcccggtttccggccatcagtgaaccggcgaacagccacgctttccgtcccataacaaccgggcgaatggcacgctcgcagatattgttgtccaggggcaccgctccgtcacccaggaagcagctcagttcatcgcgccggttcagaatatagttaatcgccttatttaatggcccgtcgggcggacagcacggctcctgctcttcaagccaggcaaacaggtcattcactatcggccgggagtaccgttatcgccactgccgtattttttccacagggcgctgtcagcgccagtgatataagacggtaattcgccatttggattgtccgctccacccaacatgttgtttccttaaggttctcacaccagaaaggacatcaacatgctgagcagagaggacttttacatgataaagcaaatgcrmmmrcmrrrmrmrtactgaggtgtactggcaatagcggacactaccatttgttctttttttaagcagccatctgatgatatttttccctgaaggctgccggggagatattccccagacgagagtgacgacgctgacgattgtagaaaatctcaatgtattcccgtattactgagatggcttcatcccggttattaaaacgatagtggctcaggctctcatttttcagcgttccccagaagctttccatcggagcgttgtcgtaacagttacctttacgcgacattgatgttttcagaccagactgctcctgtatgacccggtaatcgtatgcgcagtactgtgaacctcgatcagagtggtggattagcccggcaggtgggcgctggctcctgagcgccataaacagggctttacctgtcagctcttttgtcatgcgctctcccatggcgtagccgacaatttcgcacgtataaacatctttgatgccagcgaggtacaaccatccctcctgtgtggcaacatacgtcaggtccgccacccagacctgatttggtgctgtaggagtgaacgtctggttcagcagatttggcgcaactggcagattgtggttcgagttcgtagtcgctctgaacttgcgtttctgcttacagcgtagccttagctccttacgaagacgtgccagtcggtcacgaccaacgatgatgccattctctgccagctccgtctggagccgccgggttccatatgtttcgcgagtgcggatatgtgccaccttaatctccagttttagccgctcatcactttgttttctgtctgagggttcatgctgtacccagttgtaataaccgctcctggatacaccaaatacctgacacatcgcttcaatgggaaattgttgtcgccattgttcgattaacgcgtatttttcagcgactcctgtgcaaaatacgctgttgctttttttaatatatctcgctcaaggcgagcttcatttaacgccttacgcagttgcagaatttcagattccagttcagccaccgtgcgggaaccaggagtaccgagcccttttctggcggcggtaacccattgtcctaaagtgccttcaggaagagataatcgggaagcgccttcactgatcgaaagttgattttcaagaaccgttctgacagcttcggctttgaactctttagagtaacgttgggtttttctgctcattattagctccttctgatgccattctatttcaggaaggagtgtccgttaaactcaggctacctcatacatcgtcgatattgcgactcagattggttgctctgaacggacggtcagacgctacctcaaataccctgaaccgccagccagaaagacccgccacaaaatggttaagctgaaaccgtttatggattacatcgacatgcgcctggcagagaatgtctggaatagcgaggttatcttcgcggagattaaggcaatgggttatacgggcggacgttccatgctgcgttactacatccagcccaaacgtaaaatgcgtccgtcaaaaagaacagttcgcttcgaaactcagcctggataccagctccagcatgactggggcgaagttgaggtggaggttgccgggcaacggtgcaaagttaactttgcggttaatacgctggggttctcccgcagcttccatgtcttcgccgcaccaaaacaggatgctgagcatacctacgaatcactggttcgcgccttccgctacttcggtggttgtgtgaaaacggtgctggttgataaccagaaggctgcggtgctgaagaataacaacgggaaagtcgtgttcaactccggattcctgttgctggctgaccactataacttcctgccacgggcatgccgtccacgcagggccagaacaaaaggtaaggttgagcggatggtgaaatacctcaaggagaacttcttcgtccggtaccgcaggttcgacagcttcactcatgtcaatcaacaactggagcaatggatagccgatgtggctgacaaacgggagcttcgccagttcaaagaaacgccggaacagcgcttcgcgctggagcaggaacatctgcagccgttaccggatacagacttcgataccagttacttcgatatccgccatgtgtcctgggacagctatatcgaggttggtggtaatcgttacagcgttcccgaagcgctgtgtggtcagccggtatcgatacgaatatcgctggatgacgagttgcggatctacagtaatgagaaactggtggcctcacatcgcctctgttcagcatcgtctggctggcagacagtgccggagcatcacgccccgctctggcagcaggtcagtcaggtggaacatcgaccactgagtgcctatgaggagctgttgtgatgcatgaactggaagtcctgctgagtcgcctgaaaatggagcatctgagttatcacgttgaaagcctgctggaacaggcagctaaaaaagagctgaactaccgggagttcctgtgcatggcgctacagcaggaatggaacggcaggcatcagcgcggtatggagtccaggctgaagcaggctcgtctgccgtgggtcaaaacgctggagcagttcgactttaccttccagccgggcatcgaccgtaaggttgtccgggaactggctggtctggcgttcgtggagcgcagcgaaaacgtgatcctgctgggccctcctggtgtcggaaaaactcatctggccatagctcttggcgtgaaagcggtggatgcgggacatcgggtactgtttatgccactggacagactgatcgcgacactgatgaaagcgaaacaggaaaatcggctggagcgtcagctacagcaactgagttatgcccgggtgttgatcctggatgaaataggctatctgccgatgaacagagaggaagccagtctgttcttccggctactgaaccgtcgatatgaaaaagcgagcatcatgctgacgtcaaacaaagggttcgcagactggggagaaatgttcggagatcacgtactggcaacagcgatcctggatcggctgctacatcactcaaccacgctgaatatcaaaggagagagttaccggttaaaagagaaacgtaaagctggagtgctgaccaaaaacacaacgccaatcagtgatgatgaaatggtggaaagcggacagcatcagtaacgaaagtatttagcgggcatgaaaatggcaaataacggtcaaacatcgtggcgttgacagcgctmamrrmtrarwtwmtcaatgcggtaaagacccgcgatttttctcagctccacgcctgcacggggctccctgctggccttatacagatttgcgaagccgcgccctgcgtgactccagcaacaggctgatgtgatgccggggtgattatccgccagagatttgtacacgctgtatccgtcactcaccagcgtaccgccacaccagcactgcagccaggtctcagggtatctgagggcccgtccggtctgcgagtcaaaacagacgaccggcggaccgcttctttcaccgctgacgtaagcacacagccagccggagcaggatttaccgcctttccgggtatcaaggagcctcagggacgtttcatctgcatgaataacatcccgtgtcagaagctcatggcgtagcagtttcaccagcggggcmagtgcagctccggcaacammrmmmmwmtmmrmmmwyrwmmwrmmrrrrmrmwymmmmmmmmwwyrrrmrmmmwwrwrmwrmwrrykmwmmmkrrrmmrmwrrwmrrwmwmwwwrmwrmwrmmmmymwrrrwmmwsrctccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgtgcagttgcccggcatccaacgccattcatggccatatcaatgattttctggtgcgtaccgggttgagaagcggtgtaagtgaactgcatttgccatgttttacggcagtgagagcagagatcgcgctgatgtccggcagtgcttttgccgttacgcaccaccccgtcagtagctgaacaggagggacagctgatagaaacagaagccactggagcacctcaaaaacaccatcatacactaaatcagtaagttggcagcatcaccccssmwkmrwwrwwmmwmmwmwwkmmwmrwmrwrmwwmmwwwmmrmmrrwmmmmrrmmmmwmmkrmrmrwmmrmrymwrwmmrrmrrmmwrmrrtrgwtacatgtagcacaggtgtraaaccgccgaagcccggcgyaagccggtacggtcgtccgaatatcaccagtccccctccggctctgcgaggccatgcggcctgtcagcgatgaaaatgcctctgtgcgcggtcggagtggtcccgacaagggtttacccgcagtcggggcgtatctccgcgttagcgggccgtgagggccgcttacgagcgtgtatgagagacgtacagtagttttgtccgctcacaccagtacaaaccagtcgaaataccggtaacgacgattactcttgtctctgatgccggtggactacgagattttatttactggcgtcctgatgcggtcaggactggtatggaacctgtttatgtgatacttaatgctcttctggattccgggcgatttacgcgtaaacttattctgttagggctgtcaggctcattttcctatatcttcggctcaattgttgcgacactgggaatgggacttgttgtcgattatctgggatggggggccacatttatcgtactgatactgtctgctgtttttgccatcattttcacactgatgagtcgtgaacgctcactggagttcgaaaaagagtaattttttcaggaaaaacataaaattactgactaaatatttattttttgaatcgcagggcattccggataaggttgatatcgtgtcacggctgaagacctatggatacagtatttctggtgtggagacggatgacggatataaggcgcttgtccgcgcttttcagcttcatttccggcagaaaaattatgacggcataatggatgctgaaaccgctgccatcctgtatgcattgctggagaagtatttcccgggtaaataaggagtgagtcgtcagagctgcgatatatccccttcctccatgggctgtctcttatacacaaatccccacccggggatttgtgatgtcagatgtcccctgtaacactatgttttcattgcgctctatgaagctgagtgagcgtaacgactggagctgcttctgcggtgctccggtggcgaacgtgaagtcatactcttcgaacattttcaccgccgggaaggctaccattcgggtatacatcgcctgtttacgttgatgacgtgccagtttttcttcatgaagcatgatgcttcaagaagggcactgttgcaaagttatcgatgaggtagctgaccatcactgctgtcggaatcgatcttgcccttctgtttccagtcgcctcagcaaactatttttcagttaccacctgtcctgcggtaagcgtaaaccgaccaccgtatgtagccattagacaagaattggtaacttagacgcccatctgacatagacgaacacctaagtatggaattacaggactggcgaaaagaacctcgtaaaaagtattcgaatgaattcaaacttcgggtgatgtcttcaatcgacagaagtttctggtaatctactggtatttaaggttttccatggctaccgttactgttcattgtccgcgctgtcattccgatgaagtttatcgacatggccgtagttgttcccggcatgagcgtttccgttgtcgctcatgtaaacgtgttttccagctcacttactcttatgaagccagaaaacccggcgttaaggagcagatcgttgaaatggcacataacggcgcagggggccgtgataccgccaggacactgaaaatcggcatcaataccgtcatacggacattaaaaagctcccgccccggcggataacatctgcgccagtcgctcacgctgatgttgcccttatctgtgaacttgatgagcagtggcgcttcgttggcagcaaggctgcaacaacactggctctggtatgcgtacaacactaaaactggcggtgtgttggcttacactttcgggccacggaatgatgagacctgtcgggaactgttagccctgtttactccgttctgtatatattaggccgttggtggtaagtggatcactttttagactcgaatagctgcagttcacaaaaacaggaatccggctccgttttttgttgtcatatcagagggatatttgttatgaagaaaatatagtgagtatatatactgtcaggatacaatatgattaatggggtgtcgttacagggaactgctggatatgaggcacatacagaggagggaaatgtaaatgttaaaaaattgctggaatctttaaactctaaaagtcttggtgatatggataaagattctgaattggcagcgactttacagaaaatgataaatccgtctggtggggatggtaactgttcagggtgtgcgcttcatgcttgtatggctatgttagggtatggagtaagagaggctcccgtacctaatgaaatatcagagtatatgaccggattttttcataggcatttagagcaaattgatagtgaaggcatagtgtcacatcctaatgaaacttatagtaaatttcgtgagcgtattgcagaaaatattttacaaaacacttcaaaaggaagtgtagtaatgatttctattgaacaggccactcattggatagctggatttaacgatggtgaaaaaattatgtttttggatgttcaaacaggaaagggatttaatctttatgatcctgtagaaaaatcgccagatgcatttgtagatgaaaatagttcggttcaggtaattcatgtatctgatcaggaatttgatcactatgctaacagtagtagttggaagagtaagaggctttgctgatgctgagagcctgtacatagatttgagccggccccttgaaacgccagacagtggatgtatcttaaaataagagataggcttggatatatgtctaacactagttctaattttgagatgactggaactttgctggggctggagcttcgtaaacgtaaaactcctcaggaaaaaattgccattattcagcagacaatggagcctggcatgacggtgtcccatgtggcgcgtttgcatggtattcagcccagcctgttgcttaagtggaaaaaataatatctggaaggaagtctcactgctgttgctgccggagaggatgtcgttcctgcctcagaactggctgccgctataaaacaaataaaagagttacagcgcttgctgggtagactggccccctgaatctccagacaaccagtatcacttaaataagtgatagtcttaatactagtttttagactagtcattggagtacagatgattgatgtcttagggccggagaaacgcagacggcgtaccacacaggaaaagatcgcaattgttcagcagagctttgaaccggggatgacggtctccctcgttgcccggcaacatggtgtagcagccagccagttatttctctggcgtaagcaataccaggaaggaagtcttactgctgtcgccgccggagaacaggttgttcctgcctctgaacttgctgccgccatgaagcagattaaagaactccagcgcctgctcggcaagaaaacgatggaaaatgaactcctcaaagaagccgttgaatatggacgggcaaaaaagtggatagcgcacgcgcccttattgcccggggatggggagtaagcttagtcagccgttgtctccgggtgtcgcgtgcgcagttgcacgtcattctcagacgaaccgatgactggatggatggccgccgcagtcgtcacactgatgatacggatgtgcttctccgtatacaccatgttatcggagagctgcccacgtatggttatcgtcgggtatgggcgctgcttcgcagacaggcagaacttgatggtatgcctgcgatcaatgccaaacgtgtttaccggatcatgcgccagaatgcgctgttgcttgagcgaaaacctgctgtaccgccatcgaaacgggcacatacaggcagagtggccgtgaaagaaagcaatcagcgatggtgctctgacgggttcgagttctgctgtgataacggagagagactgcgtgtcacgttcgcgctggactgctgtgatcgtgaggcactgcactgggcggtgactaccggcggcttcaacagtgaaacagtacaggacgtcatgctgggagcggtggaacgccgcttcggcaacgatcttccgtcgtctccagtggagtggctgacggataatggttcatgctaccgggctaatgaaacacgccagttcgcccggatgttgggacttgaaccgaagaacacggcggtgcggagtccggagagtaacggaatagcagagagcttcgtgaaaacgataaagcgtgactacatcagtatcatgcccaaaccagacgggttaacggcagcaaagaaccttgcagaggcgttcgagcattataacgaatggcatccgcatagtgcgctgggttatcgctcgccacgggaatatctgcggcagcgggcttgtaatgggttaagtgataacagatgtctggaaatataggggcaaatccaamwwmrwmrmmmmwmwrwwwwwmrmwmmmmrmmmmrmwmmmwwwttgaggtagcctgagtttaacggacactccttcctgaaatmgmmwrrmmwmmrmmrrmrmymmwmwwrmmrrrwmmmrmmmkrwwwmwrryrmwmmrrmrmmmrmrmmmammwrmmrrrmwmmwmmmmmmmwmyrmwmrmrrwwmmmmrwmmwrmrmmwmmrmwwmmmrrrwmmwmmmrrmrmmrwywrgtctgaaaacmtmmmwrtmrmrwmmmrgtaamtgttrmrammmmrmtcmrmwrrmmmrmwwmwrrrrmmmrmwrmmmrmwrrwmmmrrrmmrrmrwmmmmmrwmrwmrmrrtgaargaccgtaatgggtccggagcaaagggcttgccccattgtgctgatggtgcggcggcaaccacgggcgacaatgccgatgggaggactgctgtaaaatcggcaaagccatttcctgtaagtaaacgacaggtatgggaagcttataaacgagtaaaagccaaccgtggggcggcaggcattgacgggcagacactggccgggtttgacgagaatgttacagacaatctctacaaactgtggaacagaatggcttccggcagttatatgccgcaggcggtaaggcgtgtggacattcccaaagctgacggtggtgtacgtccgctcggtattccggccgtgtcggaccggatcgcccagatggtcgttaagcagatactcgagccggtactggaacccctgttccatgcagactcgtatggttatcgaccgggtaaatccgcacaccaggcgattgcgcaggcgaggaaacgctgctggaagtttgactgggtggtggaagtcgatattaaaggcttctttgatgatatcgatcatgacctgctactgaagacagtgcagcatcatactcaggcacgctgggtggtgatgtacattgagcgcaggctgaaggcaccggtgcaaatgccggatggagcaatgctggcacggggtcgaggaacgccgcagggcggcgtaatctcccccctgctctcaaatctgttcctgcactatgcctttgatatgtggatgcaacgtcagtttcccggcgttcccttcgaaaggtacgccgatgatgttgtatgccactgtcacagccagtggcaggctgacgctctgataagtggactgaggcagcggcttgcgcaatgcgggctgcagctgcatccgcaaaaaacacgaatagtttactgtaaggatgctgaccgacggggggactacccggaaacgagcttcgactttctgggctatacattccggccgcgtctgtcgatgaatcgctggggtaagaccttcgttaacttcagtccggcgatgagtgccagagcaggaaaagcaatccgtcaggaggtgaggagaatcgcagtgacaagtccttgtacgagctggcgcatatgttcaatgcgaaaatcagaggctggataaagtactacggtgctttttataaatcagcactgtatctgacgctgaggcaaatcgatcgcaaactcgtgttatggctgccccgtaaacacaagcgacttaggggccaccgacgacgggccagccactggctggcccgtgttgcgcggagtgaaacgaggttgtttgcacactggccgttactatggggacaggcctcaatgagaagagccggatgagctgagaggttcacgtccggttccgtgagcaccagagggggaggttccctcgggtgactcgacttgtagtacattgttcacgaatgagtgatgcaacaaggctgaagaacaggttatcggtgcgcttctcggaagtcgggttggtcctgaacgcagggaagacgaatatcgcctacattgacacgtttaaaaggagaaacgtcgctacgagtttctcctttctcggatatgacttcaaagtgcgtactctgaagaatttcaaaggcgaattgtacagaaaatgtatgccgggtgcatcaaatgcagcaatgtgcaaaataacagaaacaatcaagaagtggcgtatacatcgctcaacagctgagagtttgctggattttgcgaggcgctacaatgcgatagtgagaggctggattgagtactatggaaagttctggtccagaaacttcagctatcgactgtggagtgcaatgcagtcacgtttgcttaagtggatgcagtctaaatacagactttcgaaccggaaggctcagcgaaagctggcgctggtaaggaaacagtatccgaagctatttgcccactggtatttactacgtgcatcgaatgagtgatcaagagccgtatgacgcgagagtgtcacgtacggttccgtgagcagccggagagtgaaactctctccggcggggttccgatagcggtggcgaacatgcggcggtgttgtactcgctgatcggcacatgccgtctgaacaatgtggagccagaaaaatggctgcgttacgtcattgagcatatccaggactgaccggcaaaccgggtacgcgatctgttgccctggaaagttgatctgagctcttagtaaatatcaatacggttctgacgagccgcttacgcagatcatgccataatgaataaactttaatcagcgaaattgacgaagaagctggcagcgctccaacatmmrwwmmrmmrmmmrrwymmmwwmmrmmmmmmmwrrmmrmmrmmrmmmmrmmmmmwmmmrmrmmwrmmwymmyrwmwmrmmmmymwmmrmmrrgactactttcacaaagtcacagcgactgtcagcaaaaaacacgtgatgatcgtcaattacgttatcactgccaactgacttcaccttttggttcatatgaagcagcatccagcggtgaatgttcccggatatattcgataagtacttcagcatcaataaatccggtatttatgtatccgggtctattatcaatacgtggatagccatccccaccagtagcattaaagctaaatgttgtcatacgatattttttgttaaggtcaaccgatttaccttttatttttagcatttcgatttttccttttttgactacaaagctggtattcaaaaattgtgggtaggctccggaatccggtttcatctgtgccactgtagctaggtagtcagctacttctttccccgtcaaatcaatacttactaccgtattaccaaatggctgaactttcagtatatctttgtatgtgatgttaccactctctatggaatctcgaatttccccaccactgacaacagcaaaatctgcgtcaatacgttctgtcagagcgctcagaatcagatggcccatactggtctgaacaaaacgaactttacttctgtccccttccagacgtccgttcacaaccccaaccttcacatccagttgtgctttgcctttattctggaatggtgtcagtaaggataacatctgtggatcttcagtaatttcgggggtgtaaaaagaaaattccgtttttccatcttctctggtaattctcattttgagattaactgggtgtaactgataattaaccagtttcattgtaccgttacagaattcaaaatcagcctgaccgacatattttccccactcatgcgcttgtacaatccagatgccattttgtttatctggtacacaggttgtccccggaatataattccactgttttttattatcgctggccatgcagacaggagcttgtgaatgtcctcccacaatcattgctaatgagccagtcggcaaactgcgagccatttcaacatcaccaggagcattcgaacctgactcgccattgtcatagtgacccatatgtgttgccgcaataataatatctggcttttcctgctgattaagttcatcaatgacactcctcgcctcagcagcaggttgacggaactcaatatcagtaaaatattcagaatttcctgttttcgcagtatcatctgtcgtcaagccaatgactgcaattttcaaattctgacggatgaaaattttccatggactgaataaacgcctccctgtacttttttgatatatgttagcagaaaggaaaggaaatgtggcccatttttcctgctgacgtaatatatttagaggattatcaaattcatgattaccgacggccattgcatcataaccaattagattcattccacgaatatcgggttcagccttctgaaggtctgattcaggtacaccggtgttaatatcaccaccagataataatagaacacttccgccattatttattactttctgccggatattgtccaccagagttttttgggaggataacccgtactcaccatgattgttacgccaaaagtgtccatggtggtcatttgtatgcagaacggttattttatatactttatctgtttcatacgctatagcaggatttcctgtcagtaatgacagggtgaacattattagcgtaatgtttttttttaacggtatcattgtacctgtatgaacgcatccccgtttgcaaggcttccgttgttctgacatcgacgagtagttgctgctctgtatccggccttattgcagtcagagctgcgggccactataagattcgctgacctgagccttattcccttcacggactttatgtctaggacacaattcaggtttatcagrwmacggtcttacctgtcgttgtcatcacgtcgatcaccatagccccctttcagcattcacccttctataaggtttgatgtttatcagtttcagcaattaatatggtctgatactgacgtgaaccgccccaagtttcctggagaatgttttatctgtgaactcaggctgccagatcatcgtttccgatagaagcataataagctttttctgcttctgccggagggatgtggcccagcctttccagcaatcgtcgattattataccagtccacccacgtgagtgtggccagttccacttctgcacggtttttccagctcttacggtgtattacctccgctttgtaaagaccattgatgctctccgccatcgcgttgtcatacgagtcgcctgtacttcctgttgatgccagtaatccggcttccttaagccgctgtgtgtaggatacatactgagaacctttatcactgtgatggaccgtgccggacggtcgacgggcccataacgcctgctccagtgcatccagcacgaatgtcgtttccatggacgatgagacccgccaccccacgatgtatccggcaaacacatcaatgatgaacgccacatagacgaagccctgccatgtgctgacgtaagtaaaatcagccacccacagctggtcaggtcgttctgccacgaactgacggtttacgcggtcgcctgcggcaacggctttccggctgatggtcgtacggacctttttaccccggagaacaccggcaagtcccataaccgccatgagacgtgccacagtgcatctggccactctgataccttcccgtaacaactramrmmmrmmwwwmmmmmswmymmmwkgtgattttcatcgtatacgcgctgtatctctttcttcagccagtcatcgcgctgcgcacgggcactgcgtttatccggatgatgtcgctgttgctgacagtggtaatacgttgacggggcaatatgcagttcgctgcatagcggtccgaccccgtactgctcacgcagcttatccagcagtggcatcattttttccagaggcggtcaaactccgccttcgcaaaataagcggaagcctggcgaaggatatcgttactgcggcgcagttcacgattttcacgctccagctctttcagacgctgacgttcagcggtggtgagcccwccatcaccgcccccggtatcccgctcatgctggcgaacccrgacacgcagagtctccggcgtacagccaatctttggggcaatggaacaaattgccgcccactgtgagtcatattcatcctgactttccagaaccatacgaatcgcccgctgacggacttcgggggaaaaacgagtatttttagtcatcctgtttacctctttctcaggragtttagtctccaggattcccggggcagttcagttcaggctgtacgcttccccatgtaatttgcctcgccacttcaccgggctcaggaccccgtaggatcttcaccgaaaaaaatgcatcaccagttgctttattatttgttttaggtagacctgttactaatttatacctactctgaccaagtctggataagaaaggcacacctgcacatttctacttggaaatgttttcttttaccattttgctcaaaaatttcaacatccaatattcctcgtacagaactactcaagtcctgaatagaaaaagttccagctggtacagtagagtcatatattactcgaccttgttgagatacaacaacacgtgcattagtttctgcaataccagtaatctgaggcgcataaccacgaatatttggcggtagcattcgttcatcgctttctaatgcaattcctgtgtaacgccaagcaacagcgcattctggcgcatgatctggtaaacacgtttggcattgatcgcaggcataccatcatgtgcacaaaaacccaccgagcttttacacggtgggtcccagagaaatgcaggacatcaacacgccctgcatttttattatcagaaggtatatttcacacccaaaataccttgggtgtctctgtaactgttattacccaattgctgagatacgtttccccagatgcttaggttatctgcaagacgcccccccacaccagttttgagttcaatcacattacggttgcctatctggtgatttgacatgccattcataattacaccatattgctttgagctccatttccaattcacttccaaaaatggttcgaacttacgaagagtctccttatcgatacaactctgactaattaaggatgtacgcaatcccaattttgcttgaatattattgaccccagaggatgtgaccatagtgccattgtgttcaatatagtcgtttgcttttaccccatgcctagttatctggacttgtggattgagatacaatgcgttgtcaatattattatgagcagtccagcgtatggttgggtagatatagccagcttccaaagctcctgttattcctttgctggaatatttctcaccagttaacccatctcctttgactgtattattaaaccaaccgtactgcaaccaagcatccatatatagacctgttcgttctttttcattctgaaaccaagaactgtacaaaccggcagtataaccgctcacagttcctcgtgaactataactagtatacttattatgagttttgctatcctgatatccataagctcccataatacccacatgcaagccatcatgatcagtgaagtttgtcttcaaaatatctccaccaatctgataaaccatcctgttagctgtggtttttaattgtctatcagctaaattatgctcattatgtccaccaacagtacgaatccacatggtggtttcgctgagctgacctgtagtaggatcaacaatagtgctaccgtgtcggtcatagagagaaggactaaacatcgtgttcattgctatcaggttagcaatataactgccagcttcagggcgccatgtgtgaatactaccttcacccaaattaagtgaagatacaagctgtgaactagctcggcgactactcatttgagtagactcttgattgctcatctgttttgattcttgattatccatctgactagttagataccacttattggtattcagtccagatacagtgccctgtttcaggcgatagtcataactaccagcaacaatacggcctttctgaataaaagcattcttatcagaagaatctgtcgaaataatttgaacaccttcaagagttttatctccagagcctccctcatttgtcactgcaacataggtaataccagacgcatggcctgcaatttccagccggtcagttttactgttgtcattgccgagaaccgcgcccaaagataatattccgccctttccatgccaattacctttctgaacataggtctgaccgacatttgacgaactattattgagaataacatgaccgtttttctccagagtcatgttacctgtgacaatgacggggtcaggcaaaaaaggggcattaatattattgattagcacagtcccactatttaataaagtgttaacagtttggttcatgcctgaaaaattcaaggtagcacctttattaacaataacaccagcggtacgtgtcatagcttcaactgtccccatttttagaatgcctgcattgatgttggtgaaaccagagtaggtatttttttccgccaaaatgagagttccctcccctgattttgttagggattttccgtcccagctatttatattatgatggctgagattatctgccagaatagtactaacagtaagttcagcgttttctttcagattgaactctccatgactgtcgccatctgtatcattccagctcaaaccgtagatcaatttattctggtcactgatatagctttttgtatagtcaatagcaccagtagtgagtaacggattgatgatagatatattagaaacactgaaacctgttatccctttatctgcacttagcagtttatagtccttctgcgcccagttatttataatagcttccacttttacattgttgccaaatgacatatttccagaagcttccagactactgattccagctaaatttaaagaactgttctcaccaaaagaaacgtcacctgaaacagtgaatttgttacctgatagtgaaacagttgtacaatcaccagcacataaatcaccttcaatagttggtgtggtcccagtgtaatcattgttgataattttcagaatggatgaattgttaacctgtacatcaccagtaatgacagaaccttcatggagtattaatgaattgtttccaccagtgatgtggatagcattacctactacattttgtgttccatcatcttctttacctgaaattatatatccgctattagcaatggtcatattgctaccatttatagcatcccccmaatgtccammmwwrmmrmmwmmmmwmrctmmawwgttgattatagacaggttatctcctgtgatagcattaccaccgtagccatcataaccataaccgtaagcagcacctccgttgcctccagcgaacgtgcccttattgattacagagaggctgcttcctgtgatggcatcacctmmawwacmgmmatmamcmtmammawamwwgwwammtmmmwwmmmammrrmmmwmrtmsmtttgttgattatgggtagawtgamtmmtgtgamggcatcacmtmmattmmmmmmatcactatcactataactatcgccacmmwgtccacctgaaawmmwttctccatttattataaacagattgtctcctgtaacagcctcaccaccatcaccgttatggtcagcaccgctaccgccgttaccaccaagaatcatgccttgattgattatagacaagtcagatccggtaatagagtcaccaccattaccgccacaactattaccatcattattatcaccagcaccaccatgacctgcgctaatcataatatcagagttaatccgtagcttagttctagatgcatgagaggggttggaagaagttatataccacgcatccattccatcttcaccagctcccaagggagaaagtccatcaacaggtgttaataatttttcataattgtcctctgaaaaatgaagttcttgagtacccgaaagaggaatagcaaaagctattggccccccgagcaacagggatgcaccaacaacaaaaggagaaaaagacaatttgcatgttttttcctttaccgtaggtaattctccggcccccccctgtgaacattgggtcatattacaaaaaaatttgtgaatttgattcatgcactatattatcagtaagtggttgataaacccctaaaaagattccgttttatccggaataaagggacgatatatgcaaaaatatattaaacaaagcctcaaccaaaacatgagagctgtgttatatatcaatgcaacgggagaaatttacctgtgtcttgctttgaaaaaaagagggggaaagctatatgttcgataataaattaataattttactatgatagtctgcttcagaccgcaacttttcagttctttctgtacgcttgcccaaagtttaaccactttgtttttctatacacccaatggttctatctgccccttgatgttaatccccaaagccaataacatgaatgtattcatatcacaactttcctaccagactctccaatgcaataactgcataattgtttttagtggaaatgtatcgttttcttaaagagaagaataacattccattttattatgttcccaatttaattatattaatatatattggcattaataggaaaatacatcaggagaaatcaaatgcagacatcaaacataactaaccatgaaagaaatgactcatcatggatgtcaactgtcaaatcaactacagaagtaagttggaataaattgagtttttgtgacgttttactgaaaataataactttcggcatatattctccacacgaaactctagccgaaaaatatagcgaaaaaaaactaatggatagtttctcaccatcactatcacaagataaaatggacggagagtttgcccacgcaaacatagatggcatttcaatacggctatgccttaacaaaggcatatgttcggttttttatcttgatggagataaaatacaatccactcaactttcatcaaaagaatataataacttgttaagttcactcccccccaaacaattcaacttaggaaaagtacatactattacagctccggtatcagggaattttaaaactcacaagcctgcaccagaagttattgaaacagccattaattgctgtacatcgataataccaaacgatgattacttccccgttaaagacactgattttaatagtgtttggcatgatatttacagagatattagagcatctaacagcaatagtacaaaaatatatttcaacaatattgaaataccactaaaattaattgcagatctaattaatgagttaggaattaatgaattcattgatagcaaaaaagagcttcaaatgctaagttacaaccaagtaaataaaataatcaattcaaacttcccccaacaagacctatgttttcaaacagaaaaacttttgtttacttcactctttcaagaccctgcatttatatctgcactaacatctgcattctggcaatctcttcacatcacgtcttcctctgtggaacacatatatgcccaaattatgtccgaaaacatagaaaacaggcttaatttcatgcctgaacaacgagttattaacaattgtggacatattataaaaatcaacgcagttgtccctaaaaacgacactgcaatctctgcctctggggggagggcttacgaagttagctcatcaatattaccatcgcacataacatgtaatggtgttggtattaataaaatagaaacaagctatctcgttcatgctggaacacttccaagcagtgaagggcttcgcaatgctatccctccagaaagtcgtcaagtatcatttgccatcatatctcctgatgtttaagccaatattatattggctgccagactgtaaaaaaacgttcttcagaacagctctataaactattaaaccaaagctaaaaggatggggaccaatagaatctcagcccatcctttttatatcattatgaaccgccccggaaatcctggagactaaactccctgagaaagaggtaaacaggatgactaaaaatacacgtttttcccctgaagtccgtcaacgggcagttcgtatggttctggaaagtcagggcgaatatgactcacaatgggcagcgatttgttccattgccccaaagattggctgtatactggagactctgcgtgtctgggtacgccagcatgagcgggataccgggggcggtgaggtgggctcaccaccgctgaacgtcagcgtctgaaagagccggaacgtgaaaatcgtgaactgcgccgcagtaacaatatccttcgcctggcttccgcttattttgcaaaggcggagttcgaccgcctctggaaaaaataatgccactgctggataagctgcgtgagcagtacggggtcggaycggtatgcagtgaactgcatattgccccgtcaacgtattaccactgtcagcaacagcgacatcatcatgataaacgcagtgcccgtgcgcagcgcgatgactggctgaagaaagagatactgcgcgtatacgatgagaatcatcaggtatacgctgtgcgtaaagtctggcatcagttgttacgggaaggtatcagagtggccagatgcactgtggcacgtctcatggcggttatggggcttgccggtgttctccggggtaaaaaggtccacactaccgtcagccggaaagccgttgccgcaggcgaccgcgtaaaccgtcatcaaggcaatatgccgcgtactccgggtggcccgcagcggctggtatacgtggtgtcagcggcggacaaggataaacacacgtcagcagttccgtcagcactgcgacagcgttgtccgcaaggcttttacccagtccagcgttacggtgccccacgcctgacggatgaactgtgtgctctggttaccactttaacgtaaaaaccgtggcagccagtctgcgccgtcaggaactgagcgcaaaagcctcccagaagttcagcccgatcagttaccgtgcacacggcctgcctgtatcagaaaatctgctgacgcaggatttttacgccagtggcccgaaccagaagtgggcgggagacatcacgtactattacagctcaccgactgcggggaaacacggggcgccgggttattgaaggtgacctgtccagctacttcgacacagtacatcaccggctgctgatgaaagccgtctgccgcaggatcagtgatgcacgttttatgagactgctgtggaaaacgccgtgttgatgttggtctcttccgggcggtcagtgaaagtatgccacagggcggtgtgatctcgccgttgctgtcgaacatcatactgaatgagttcgaccagtatctgaataaacgctacctgagtggaaaagccagaaaggatcgctggtactggaatcacagtatccagcgaggccgaagtactgctgtcaaagaaaactggcaatggaaacctgccgttgcttactgttgctatgccgatgactgtgtgccacgaaggcgcgtcctggggacgtgagtccctgtgcagccatgcggcacaagagatgagggaaggcccctcggtgtcggcgtgcaggcgcaggcaccaaaccatccactgctgctgcatttaagagatgtggtggtgagcgagatgggaaaaggtcggacgtgagccgatcaggtatggttcgaagagacgaacgamagtgaaccgctgatgatgtrwmramagmmwmwmratgatgtcamaacaatggtggagtcgttcctgtgggatatgtttactgcctgcgcggcatccggcataaaagcggcgtgaagtcgattcaggctcttgtggggaacgtggacctgtcaccccgatgtgaagggagaagcgcaagtggaagccccatgagcgccagagtaccaatgcggggtacagggacggagcaccaagcgattgcgcagtaaacatgcttgacaacttaattggggccccctcttccttttatggtgtaattgtttgattgaatacttccggggatttcagtatgaaatgaagtatatttaatatactttacaaaaagtttgtattagtggaataaggacaatagtttatccaaactgccaagcccctttattataagtaagatctggtactaaataagcgttctaaaccgccccgagtaggcggcctgacggctgcggcttgtcatggcctgtgattaccgttataaaaacagacaatatcattgtctttcaggtagttatatggcccgttcagctaaaccacgcaaacgaaaacctgcacctcaaagaagcaaactcctccgctatgtcgtaaaacttcacgaagatgatttttttgacgaagaagaagctgaggttctgcgctttgataattttgacgatgccgttgagtgttgcgcagacctgaatattccgttctttgtggatgccggaaacaaaaaactggtcttctggtttgtacgtgttgatgacgaagggtatccggaaattgcccgctgcacggagcgggaatttgcgacaattcctgccggtatcagcgccgacggcatgtattgccctgaatgcggtacggttcactggccggatggcgttattccgcctttctgatgcttccccgttttgccgatatttttcagcagggaaaccgatggcttaactggctggagaagcagccggtgcagatgagccgcctggaacattatgccggtcaggatgaaattggcctgcgttacaacagccaccggacaaaacgagaagaaaaccttgtgatgagtggcgacgagtttatggaaaggttctcctggcatgtggcggataaggggttccggatggtgatacggggtcctgagtccggtgaagcggcgattactggaagatgtggtgtacgccataacggagacagtgagaaaaacggcgaggcaaatcacaaggagagggatgtatcagcagtgactgaaggttgacccgctgaagtgcgagctgtgcggaggtcagatgcggtttacggggctgaagaggggttaccgtctggcagagcaggttatgatgcatgagccgctggctcgaatacggtggtgcggctgagagtcgcagaggggaagttgcgtccatgttkmrrrrmmmrrmrmmmmmmmmmmwmagagatacattgaatcaatcaatgtaagcaatattaagtgtctataccgattacttgatgatcaggttgtgttgaataaacgcgcattcttcggtgcttttttaatcttttggggatttaaattcctatcgatggacatgaatgcaggctatataagagcagcgcgcatccacttacccaatgcggttgagaaaatagccttcgatcgctttcatgtggcgaaacaaccgggtgaggtggtggataaaacccgccagaatgaaccgccccgggtttcctggagagtgttttatctgtgaactcaggctgccagatcatcgtttccgatggaagcataataagctttttctgcttctgccggaggagtatggcccagcctttccagcaatcgtcgattgttataccagtccacccacgtgagtgtggccagttccacttctgcacggtttttccagctcttacggtgtattacctccgctttgtaaagaccattgatgmtctcygccatcgcgttgtcatacgagtcgcctgtacttcctgttgatgccagtaatccggcttccttaagccgctgtgtgtaggccagcgatacatactgagaacctttatcactgtgatggaccgtgccggacggtcgacgggcccataacgcctgctccagtgcatccagcacgaatgtcgtttccatggacgatgagacccgccaccccacgatgtatccggcaaacacatcaatgatgaacgccammtmrmmrmmgmmmwrmmwtgtgtcaacgccacgatgtttgaccgttatttgccattttcatgcccgctaaatactttcgttactgatgctgtccgctttccaccatttcatcatcactgattggcgttgtgtttttggtcagcactccagctttacgtttctcttttaaccggtaactctctcctttgatattcagcgtggttgagtgatgtagcagccgatccaggatcgctgttgccagtacgtgatctccgaacatttctccccagtctgcgaaccctttgtttgacgtcagtatgatgctcgctttttcatatcgacggttcagtagccggaagaacagactggcttcctctctgttcatcggcagatagcctatttcatccaggatcaacacccgggcataactcagttgctgtagctgacgctccagccgattttcctgtttcgctttcatcagtgtcgcgatcagtctgtccagtggcataaacagtacccgatgtcccgcatccaccgctttcacgccaagagctatggccagatgagtttttccgacaccaggagggcccagcaggatcacgttttcgctgcgctccacgaacgccagaccagccagttcccggacaaccttacggtcgatgcccggctggaaggtaaagtcgaactgctccagcgttttgacccacggcagacgagcctgcttcagcctggactccataccgcgctgatgcctgccgttccattcctgctgtagcgccatgcacaggaactcccggtagttcagctcttttttagctgcctgttccagcaggctttcaacgtgataactcagatgctccattttcaggcgactcagcaggacttccagttcatgcatcacaacagctcctcataggcactcagtggtcgatgttccacctgactgacctgctgccagagcggggcgtgatgctccggcactgtctgccagccagacgatgctgaacagaggcgatgtgaggccaccagtttctcattactgtagatccgcaactcgtcatccagcgatattcgtatcgataccggctgaccacacagcgcttcgggaacgctgtaacgattaccaccaacctcgatatagctgtcccaggacacatggcggatatcgaagtaactggtatcgaagtctgtatccggtaacggctgcagatgttcctgctccagcgcgaagcgctgttccggcgtttctttgaactggcgaagctcccgtttgtcagccacatcggctatccattgctccagttgttgattgacatgagtgaagctgtcgaacctgcggtaccggacgaagaagttctccttgaggtatttcaccatccgctcaaccttaccttttgttctggccctgcgtggacggcatgcccgtggcaggaagttatagtggtcagccagcaacaggaatccggagttgaacacgactttcccgttgttattcttcagcaccgcagccttctggttatcaaccagcaccgttttcacacaaccaccgaagtagcggaaggcgcgaaccagtgattcgtaggtatgctcagcatcctgttttggtgcggcgaagacatggaagctgcgggagaaccccagcgtattaaccgcaaagttaactttgcaccgttgcccggcaacctccacctcaacttcgccccagtcatgctggagctggtatccaggctgagtttcgaagcgaactgttctttttgacggacgcattttacgtttgggctggatgtagtaacgcagcatggaacgtccgcccgtataacccattgccttaatctccgcgaagataacctcgctattccagacattctctgccaggcgcatgtcgatgtaatccataaacggtttcagcttaaccattttgtggcgggtctttctggctggcggttcagggtatttgaggtagcgtctgaccgtccgttcagagcaaccaatctgagtcgcaatatcgacgatgtacgcgccctgctggcgcatttgctttatcatgtaaaagtcctctctgctcagcatgttgatgtcctttctggtgtgagaaccttaaggaaacaacatgttgggtggagcggacaatccaaatggcgaattaccgtcttatatcactggcgctgacacatgtgctgacgtaagtaaaatcagccacccacagctggtcaggtcgttctgccacgaactgacggtttacgcggtcgcctgcggcaaaggctttccggctgatggtcgtacggacctttttaccccggagaacaccggcaagtcccataaccgccatgagacgtgccacagtgcatctggccactctgataccttcccgtaacaactgacgccagactttacgcacaccgtataccttgtgattttcatcgtatacgcgctgtatctctttcttcagccagtcatcgcgctgcgcacgggcactgcgtttatccggatgatgtcgctgttgctgacagtggtaatacgttgacggggcaatatgcagttcgctgcatagcggtccaaccccgtactgctcacgcagcttatccagcagtggcatcattttttccagaggcggtcgaactccgccttcgcaaaataagcggaagcctggcgaaggatatcgttactgcggcgcagttcacgattttcacgctccagctctttcagacgctgacgttcagcggtggtgagccctccatcaccgcccccggtatcccgctcatgctggcgaacccggacacgcagagtctccggcgtacagccaatctttggagcaatggaacaaattgtcgcccattgtgagtcatattcgccctgactttccagaaccatacggactgcccgttgacggacttcgggggaaaaacgagtatttttagtcatcctgtttacctctttctcaggaagtttagtctccaggattcccggggcggttcagaatgaacatccgcatcttcctgttgaaagtcgtcgtcaggcaaagggaactcgcttcctgtggcagcacagcgataaatggatgaccgaatccaggcaggaaaagctgatatggctgcgtgcgcagatgaagctgacgagcctgtgctgggcgctgaaagaactggcaaaggatatctggagcagaccatggagcgaggaaagacgcaacgactggcagagatggctgtcgctggcggccaacagtgacgtccccatgatgaagaatgtggctaaaacgatagggaaaaggttgtacggtatcctgaacgccatgcgccatggcgtatcaaacggaaatgcggaagcactgaacagcaagatcaggctgctgagaataaaagccaagggctaccgcaaccgggagcgctttaagctgggagtgatgttccactatgggaaactgaatatggcgttctaagccttcccaccatgatcggggaagacccagatatgaacaggcacattcagagttactttaaagagcttactgaacgtctgaggtgttagagccatgactgccgcagcattgctggcggaaatgcctgaatcgagcagcctctcgagacgtgaaatcagcgcacttgtcggtgttgctcaggtaaaccgggactcgggaactctgcgcggtcgacgaaccattttcggtgattgtgctggggaagaacagctctgtacatggcggcgcttgaggcaccccggtttaatctggtgataaaggcgttttacatgcgcctacttgcagccggaaacgccaaaaaagtggcactggttgcctgtatgcgtaagttgctgaccattatgaacgcaatgctcagaaaaaacgaagaatggaatgaatcgtatttatgaaaactggattgtgggccataaatattccaaaaagatagcccgctccgtttattaccggaacgggcgagagcatgacattgtctgtaattataatgagactgctgattctacttatttttaatttcaagaaaagcgttttctacaaagaattatgagatttaatgttttgtttagacaatattatcattctgttcaataatgaaagtcttcatcaacaacaagtaaaaacaacgtccctctcatcgaaccggacttgcagatttcccgcatccggctctcttgcaacacatcagaccttcacgttcgggcggttatggggaaggtccagcaagctgtatgagccctaactgtttgaataccagtagattaccagaaacttctatcgattgaagacattaccaactagcgttactaaatgttgaccaagttttgttcacttgtctggttttgatagtagcattctgcagagtattttgatatgataaatatgcattaacaatgatattattgttaatttcctagcatgaaaattaaaagatcaactttttatctcgaatattttttacattatcagttggtttcttatgaatgacaattcattgcttagaaattcttcattattcatcgcctatatgggatgtgtagggtgggtcagtgcttattcttatggctggggaacctctttttactatggatttccatggtgggttgttggtgcaggtttggatgatgttgccagaagtttactctatgctatcatagtaatgggtatactttttaccggttgggggattggtattttattttttcttcttataaagaaaagaagtaaaatacaagacttgagtttttttagactattttttgctattaccctactgtttttccctgtaatattcgaacttttaattctcaaacaatatttcatacttccattatcactttcatgcattattagttcattggttatttcaataataattcgtatatatggacggatcttttctgtatcatgtttttcagatatcccttttgttagagaacaccgcattaagttaattatggctggttttttggtttatttttggcttttttcctttcttgttggctggtataaacctcaattaaaaaaagaatatcagatgctttgttataataatagttggtattatattcttgctcgttatgattcacggctcgttttgtctagttcatttaaagatgacagtaacagattcttaatatttaatacagaacagtcgggattttatgaaatcaatgatgtttatgtcagaaagtaataatcctctcccacaaagtgaattaaatgaatatataatgcgttcgaagtatatgtattgagctgtcatactgagaaacaaaccttagggcagcacaattaaagccagaaacaactatgcaagaaagttactaaataagtgctcggaacagtaaaacactcaggggggactcagtccggattatgtaatcagataaattgccaaacatcacaaatcaccagccggactgagtgatgccgagcattgcaccaataccccgtgatgaacgatgcctgatgcagaaagctccctataaaacaaagaacaaaaactacgaccggcgattcaccgtcatactgatgctgcatcgtggtgacactgtctgtcactttcatgttgactgaacaactctgctgcgaagaaggtgtaccgccggcgggatttgattgtccggcgtctagcttccacttccctgatggaactgaaagttgttatggccggtcagcttgccctgtccgcaggacatgaggtgctgtgacggacagcaccagtaacggaaagtgacaatgaaaatgaatactgcgctgatagttgcactaatgtgtatgtggtatgcggtacccgctgctgcaaaagagactttactggctatgccacgtaattcaacagaacattgctatgcggagattaatgtgcacggaccatatggggtgtatttccgagtggtcccgcatccccccgggggaaagagctgggtcgaatgtaacagtgactactattactctgataaaccaccgggagtacagatactcggaacccgtgcagggtgtcgggtatacggaatctgcgggaccacctcgacactgcatgtggctggaagaggggttgtgtgtattaaaaatatatgcagtccacgtgggatgataattcacaggatacggaaacgtcctgtagtggctgtaagcgatgagatgtgatcttacccagcaatagtggacacgcggctaagtgagtaaactctcagtcagaggtgactcacatgacaaaaacagtatcaaccagcaaaaaaacacgtaaacagcattcgcctgaattttgcagtgaagccctgaagcttgctgaacgcatcggtgttgctgccgcagcccgtgaactcagcctgtatgaatctcagctctatgcgtggcgcagcaaactgcagcaacagatgacctcgtctgagcgtgaaagtgaactggccgctgaaaatgcccgcctgaagcggcaactggcggagcaggcagaggaactggctatcctccaaaaggccgcgacatactttgcgaagcgcctgaaatgaagtatgtctttattgaaaaacatcaggctgagttcagcatcaaggcaatgtgccgcgtgctccgggtggcccgcagcggctggtatacgtggtgtcagcggcggacagggataagccctcgtcagcagttccgtcagcactgcgacagcgttgtcctcgcggcggcctttacccggtcaaaacagcgttacggtgcaccacgcctgacggatgaactgcgtgctcagggttaccactttaacgtaaaaaccgtggcggcaagcctgcgccgtcaggggctgagggcaaaggcctcccggaagttcagcccggtcagttaccgcgcacatggcctgaggtgtactggcaatagcggacaccaccatttgttctttttctaagcagtcatctgataatattttatcctgaaggctgccggggagatattccccagacgagagtgacgacgctgacgattgtagaaaatctcaatgtattcccgtattactgagatggcctcatcccggctcttaaaacgatagtggctcaggctttcatttttcagcgttccccagaagctttccatcggagcgttgtcgtaacagtttgaggtagcmwrmswkmrwmwwmmrmwmymwwmrmwmmsrrmwwrrmmwyrmmrwwmrmwmmkmrmwwmrmmwmmmwwmrmwmmmmmrrrmmmywmmmwwkwwmwrmmrmwwwwwrmmwmrwwwwwwyrmwrmywwmmmmwrwyrrrrrmwwwwwmswmmrrrmmwmwsmmwrmmrrmmmmmwmwrwrwrwrwwmwrwmmymrwmwrwmmrrrmwrrmmwmwrmwrwwrwwrrrwwmwmmmwmmrmmmmwrmmmwwwmwmwmmmrrrmmmwrrmwwrsymswmwmryrmwkmwwwrwmmrymmmrmwmmwmwrwrmwmwmmmrmmrrwmmwwmmmmwrrrmrwwmmwwmmmwrwkmmmrmmmmmrmwyrwmmmrkmywmrmrmmwkwmwwwwwwrmmmmyrwwwmmrmymwwwmwrmmmmmwrmymwmmmmmmrwmmmwrmrmmmmmwrwwmmmrwmmwmrmmmrrwymwmmmrwmmmsrmsrmwrwmwwwmwrkmkrmrwrmmwrkwmrmmmmrmrmmwwrrmwmwmmmrmwmwwmmmrmmwmmwrwmrrmmmrrwwmwmmrwmrrwwwwwmyyryrrmwwwwrwmrwrrymrwwmmrmmrkwmmmwmmwmwrwwmmrmmsrmmrmmmwmwwrmrrmmsrrwrmmrmmrsmmwymmrkwrwwrmmwrmwmwwwmwrrmmrrmmwwwywwmwmmmmmrkrmmmmmmrrmwrwwwkmkmwrmwmmrmmrmwmrwmmwmmmmmmrmmrwswmmrmmywwmmwmrmmmrmmmrwwmrwmrymrrwmwrmrrmkwwwmmsrwwrmsrwrwmmrrmymmmrrmmmymrsmrrrrrwwkmmrwwrrrkmmrwwwrwwwmrmmrrwwmwmrmwmmymyrrywrwwmmwwmwrwmrrwrrwmrymrmmmrrmmmmmrmrrrwwwwmryrmwmwrmmrmmwmwrmmrrmwrwwrwwmrmmmwrrmrkrmwmmsrrrmrmwymmmrwwmwmmmrrwrmwrmmrmmsmmsmwwrwyrmwmmmrmkwmwswmmrrrryrwwmwmrwrmmrrrmmmyrwmmmmrrmmwkwwmmmrmmkwwmmrrrmmrwmywwwwkmyrmmrwrmmmrmmrmmmmmrrmmwrmmmrrymmrmmmrmymmrrwwrrrrwwwrrywwymmrmmwrrmwwmmmmmmmmmrrmmwrwrrmmrrmyyyskmmkrywwmmmwrswmrmwmmrrmrwwmmrrkrwmmmwrwmmmmwrmmsmmrrwrrmrwkmkrrmmwrwrmmrmrrmwwrrwwrwrwrwwwmwmwwmmwrwrwrmwrmsmrrmyrwwrmrmymmmmmsyrymsmwkkwrsymrmrmwrwmmymmmrmmwrmmmmmmmwkmmrwrmmrrmsrmwrmmmrmmsrrwwmrwrwrrmmmrrmrwmmmmrwrwmrrrmmwmkwmmmmrwrrwmrwmmsssmmmmmmwwmrsrrmwwwrwmwwmmkwrwmywmmwrmrrywmmmrmrrmrrmrrmrmrmrykmwsrwrmmwmmrrmmwryrsmmrmrwwmwrmrmrrrwwwrmwwmmsrwmwmymywmrwwrrwmrrwwmwwmrwrwmmrwmmrmmmmymmmrmmrrmrysmmwwmmrmrmmmwrmrmrwrwrrmmrrwwwsmwymmrrwksmkrrmmmsmmrmmmwmrmwsrmmrmwrmrrrmrmmrwwwkmkrmwwwwrrwwwmwmwwwmrrywwrmrmrrrmrrrsmrrrmwmrwmmmmrwrmmmwmrwrmwmmrmmmrmrmmkrmwmmmwrymrwwrmwrmmmkrmymrwmrmrmwmsrmwwwywwmrmwrmrmwwmwrmrkwwmkmrkrrmwrmrmmsmwwmrrwmmrmmkmmmmrkmmrrwmwrrmmmmrrmwrkrrrmmwwwmmmmmwrrmmwymwrwmmyrmmmwwmkmmmrmywmrmmmwmrrwmwmrwyrrmyrmmmmymwymmmrmmmmrmmrmwwkmmmsrmrrmrwmwmwmwmmrrwrwymrrmwwmmwmmmmrrrmmmrwmrrmmmmmmrmmrwmrmwmmmrmrmkwwkmrmmmrmmmrmkrmwmmwrymsmmwrrmrmrrmrwmrmrwwwwwwwmmrmwwmmrrwmrmmmmrrymrmmrwryrrrwmwmmmmwmrmwwwrwmrmmmrmmwrwrmmwwkwmywmmmwmmmmmmmmswkrryrmrwmmmrmmmrrwwkyywmwwwwyyrkmwmrmmrwmsmkmmwrkmmrrmmkwmmmmrmwmwrmmrmmrmrrwrwmwwwrrkmrrrmmmmwrmrwwwmwmmmrmrwrwmmwrrrmwmwmwmmrmmrrwmmyrrmmmyrmsmrmrmmrmrkmwwwkmmryrrrmmmrmmmmwmmmmmmsrrwwrmrrmrmwrmwrwrwmmmkwmmrwmmrmwmmmmmrrwwmswmmrmwwmwwmwmrmmmwwrwrmwrmmrkmwmwwmrwmmmmrwywmmmwmrwrmwmmwrrmwwwwmwmwwwwwrkmmmwwrmmrwmwmmmmwrrwwymmmmmmrrmmmwrrmrmmwrwrrwrmmmwrmrkwmmwwmmmrrmryrmmwrrmrmmwwmmmrwmmmyrmwmrrmmrwmwrmmmmmwmrwrmwmrmwwrwswyrmmmmmmwryrmwmrrmrmwrkmwrmmmmmwmrwkmwmrmwwskrmwrmmrmmmkrsrmwmsmwwmmrmwrmmmwwwwrmmsmctgagtttaacggacactccttcctgaaatagaatggcatcagaaggagccaataatgagcagaaaaacccaacgttactctacggagtttaaagccgaagccgtcaaaacggttcctgaaaatcaactttcgatcagtgaaggcgcttcccgattatctgttcctgaaggcactttaggacaatgggttaccgccgcaagaaaagggctcaatacaccaggctcccgcacagtggctgagctggaatctgaagttatgcaactgcgtaaggctttaaatgaagcacgacttgagcgagatatattaaaaaaagcaacagcgtattttgcmcaggagtcgctgaaaaatacgcgttaatcgaacaatggcgacaacaatttcccattgaagcgatgtgtcaggtatttggtgtatccaggagcggttatwmcgaccgggtacmrcmtgccccctcagacagaaaacaaagtgatgagcggctaaaactggagattaaggtggcacatatccgcactcgcgaaacatatggaacccggcggctccagacggagctggcagataacggaattatcgtcggtcgtgacagactggcacgtcttcgtaaggagctgaggctgcactgtaagcagaaacgcaagttcagggcgaccacaagctctgaccacaatctgccggttacgccgaatctgctgaatcagaactttacgcccacagcacctaatcaggtctgggtcgcagatataacgtatgttgccacccgggaaggatggctgtatctcgmwrrmrwmmmmrmwrwwkmwmmrwrmrmmmwwrkmrrwwmmrmmmkrrrmrmmmrmmwrmmrmmmrmrywgmmgggtaaagccctrttwmwrgmrmtmmrrmrmmarmammmammtrmmrgrmwmmwmmmmmrmwmyrmwmrwrrwwmmmmrwmmtrmgcmwacgattaccgggtcatacaggagcagtctggtctgaaaacatcaatgtcgcgtaaaggtaactgttacgacaacgctccgatggaaatcttctggggaacgctgaaaaatgaaagcctgagccactatcgttttaagagccgggatatttcgtcagcatacgggaaaactgattgaggtcggcctcagttttgaggcctttagccagttcagccgcaagggctttaagcttcttttcgtccataatttacctgtctccgttgttggagtaaacatatcaaaaacaggcaattacacaatttaaattacagtctcaaaacaaacccaacggtcaatatagtttaccagcaagcttactagcaaagtgctaatcatttggtatctttcgtgctattctccttttacgtaagtacttaaggggaacgtatttactacttttaaagaaagctccgttactatctgaaatgcaatgagataataaacgattactctcggttagtatctcatgatcatttacagactttgcataatcgcgaatactatataacagagttttaacattacaggatgaatagtctctcttatcaactcctaataaaagaaggattatggtcagtcgtttttgctgtcttatcatatattatttagtgcagttcctaacaaaaacataacaattttctacaattgatcctgtgactacataaatatcttcaacaaaaatatttatatcagcaaaagagttattatgaatgattataaatcatcaggtgttttttcaacgaccaactccagagcacaacgataaatgtcttttctaaccaggtcacattcagatgaaatctttctgagtaaaacatttattatatccttattgctaaggaaagatattgggttcaatacctcattagcaatggagttaatgatggaagattctatttcaaattcagactgatcaatacctattttattacagaaattaactcccatataacacatctcctttacttattaatgagtgttttcaacctgctcaagccaaacaataagacgtaggacttcatccaagtgttcaaaatcaacaaaactatattttgtatgtgttttatatagttttctagctaatttcacatcacgcacagttggtataccaacttcatttgcatattttctgacagccaaggcacactggttagtttcaatgagagaaataaaaggtgcaggcgctatttctggattaaaataaataccaattgcaatatgagtcgggttcattaccactaattttgaattacgtatatcagatttagtctgctctgaaagaatctcgatatgcaactcacgccttctcgactttgtctcaaagtgtccctcttgctctatatattctctttttatctcctgtttatccatcatcatatctttcatgtataaaatgaactcaatcacaaagtcaagaataataacaaaaatagaaaatgccaagaaaaataatattatatccttaaacagccccccccaaataagatataagccatcaacactagaaaacacctgagaaaaaattatttttcggtcattaatccaaaagaaataggttgttaatgctagaataataagaagcagaatgcttttgaaaaattcttttattgtctttatactaaatattttttttaaccctttaacagggtttaatactgaaaaatcaatcttgatagctttagtcgcaagaacaaactttgtttgaaccaatgttggcaacacagccgaaagaacacagaaaaaaagtgggaagccaattatcttaaaaaagacaataaccactgcaaaaaagtatttaccctcattaatttcgaagtcattaattattacatatctgtataagagcattacatcacttaaggaaaagaatgatattatagtaaatgtccctaccagaataataacaacagtcgttaaatccttaaatttaaatgactgtcctttttttgcggcatcctttagttttttaggtgtcggcttttctgttttatttgccataatattttatctaacaaatagatttgtgaaaaatttatgttcaccgaggaaaaattgaactttagaaaagtaaatagtagaagaacagatgaaaataataaatattgcaagtaaacttttaatagttaatgatacggaaaaagcattcatctgcggagcaaatctcgataatacaccaagtaatacttctgatagtaacaatactatcataacaggactggctaaaataacagcctgacttgccaataaagtcagaaatgttaagatatttgagacacggaaagaacattgagaaaataacgggcatatattataagacaattgtatggattctaaaataaagaccataccaccactgtatagaaatacaactgcagaaaaaagattgaaaaattttgccaactcagacgtatcaacaccattggcaggatcaattgaactactaagcgttgccccacgctggttgtcaataatgctaccaacagcatgaaatatccaaaagggaagagacaagcaaaaagagagaaggaggccaacaattatttccttgaacataaggaaataaacatgttcaaaaacagaagaacctatgtctaccttaccagaagtaattaatcctgatgcaacaagaaaaatcacaggtattctaatagatggagaaattataccgttatttaaaaatggaagaaagaaaaataatggagccaatctaaaaaaaacgccgttcaggagtattaaaaacacatgaatactttcgaaccagcttgaaatgtccatcaaaccccactcttaattaaaaacattatttcatgacaaaaagacaataaaacctcaccataccatccagaaagaagtagcaaagatattgagacacctataagctttataccaaaaggaagtgtctgctcttggagttgagtcactgtctgtaacaaaccaatagataatccaataaccgtagctatacctactggccataaggaaaagataaggattaaataaagagccttattacccatataaactatatcagacacattacctccatcatctaagcaggaacattgatatattgttcaatcaaggccttagacaggataccccacccatcaagcgcaacaaataatacaagttttattggtactgatattgtgattggactcatcatcatcataccaagtgcaagtaaaatgcttgaaataacaagatcaacaacaacaaatggcaaatataaataaaaacctattttgaacgcatctttgatttcacttagcgcatatgcaggcaagagggaaaacaatgaataatcattattctctgcactctttaaatcagcattttcttcttcagacctttggaaaaaacgtgctaactcaagatctgtatgctttttcaagtattgcttatattccatcaagccagaatcagaaaaacggacaatatcagatattgtatcaaatttttgagggccatttagataattttcataaccagcttcaataatgggtttcatcacgaacaaagccataatcaatgcaataccattgagagtcatatttgatggaacctgctgcaaaccaagtgcatttcgtaccattacaaaaacaatagaaaactttatataacaggtaccagcagcaactaaaaatggtaatagcgtaaaaaaagaaagtgtagcgatgagggacatgtcactcagcatgagattactcctttaccatccaagaactaatctcgataccataaccatcctcaatagaaacaagttccccatgcccaaccaaagaaccatttactttaatatttacatgtttaactatgtcatcgggaaacttgaataattcgttttctacatacatttttagttcattgattgtcatatttttttctaaaagaataaagtccacttttacattgatatcatcataattgaataatgccaaagaaacttctgtgtgttctgactcaccattactttctgacagattaatttttgcctcattattatcattcacaatataatccccaataataacttgattacacaataataaattataaagtttctgaactatgattatatcaccagtgcgaattctttttaaagtagccagccttacattagatgaacctaagcaaaactctaaaagatgatgcaataagtcaagagaaacttgattacgtaataccggcatcttgtcaaaataaataatacctaaagaagaggaaaaacagcataaatgactcccagtagttgcatactggatttcattcgtgatccggaaagtaatcttatcagacagttctggaatcgaaaatacagatgacgactctataaagacacattttgataaatgcagtaaagattcttgtgtagaaaatcctgaaaatacctcgtatttttttttcaaaaaagataatgtatcaatacgaattacccaatccccacacacggaagtaagagtgactaaactttcactgcctgtaaatttatttttgaatgtctgaatcgaaaaacgctcgcagagttgctttgcatataaaatctgtagtttttcgttagcgtcaaaatgtttaattcttagcattcttcttcactctgttcattatatcctgtactattatttttattccccttattatgttgactgctatcaaatctataattaccctcagcatcttgtttcaaaattaagtcaagtttatttcctacaaattgatctgacggttttagaacaaaagaaccagactccactgaaatactaacagaatgtccagccccccactgtttaaagtggtaagtcatttcactatttttttgaaaaacatttttatcattttcaactattgatttttcttgcaatgacatggatggggttatgtctgcagtatttttattcattacaggcagctcaaatgatttagcaacacttccatttagtaaagaaatgatgtcagcaatattcttatcaatagacaagctatgattttttaaaccaatttgaccatttctattatcagattctagcagattatcagggtttacagaagaagtatgacttctttcgaagttcttgtctgcatttccactcttattatatgaaaagatccccctcatatcgccgatatttattttttcccttgcttttatatcttgatctggtatcaaaatattttctttaatggccattgattctggaatatcagaatatgtcgctcgttcgtttctaagttcgaacgcatttttaatgccactataaatttctcctttatcatgcttatgttcaaagtgcttatcaatagtaaaatagttagatgcaattaaagagtactcaggaatttcaaccttcctcttcttcttcaaaacgagactatcaatattatctattgaagatagtttctcacatttttctatctgtttgtcactactaaaatttaggtttatattatctaatgccatacttcatatcctcttgtgataactcatcaagtatcaactgtttggcaaagttatttcttttaatcaagccaatccgtttacatattttactctctttatgaagcagttttcttttagaagcatttgcctcatcaatatctttttgaatttcttcaattcttccctttagaagaattcgttcagactgatattggataatatgcttttttactatagcgatcttacgtcttgtattgaaaagctgactaacactctgagcgcctacaggttctaagtcattctccagttgcaggaagtatataatccttttatctaatgcctccacatctcgatacaaagagttttttttttagtgttaatatgtgtgcttcaattaatttaacagaccgttgatatttatctttgatttttaaaaccttatctaattgtttcaccaataagctccattgtctgctcataagtaaaaccgagtcgataatcttgcttcagaaaactctcaacaacagatattttgttgtagattttatcctgagaggcattctcccccggtttgtattcaccaaagtcaataattgtccttagctcttctatttcagaaagtagctcccgaaatgcagcggccataatacgatgtttttcatcaacaacctgtgtaaataccctgcttatactttttaaggaatcaattgcaggaaattgtcctttttgggctagatttctgctcaaatatatatgtccatctaaaatggatcttacctcttcagctaatggatcagcaaaatcatcatcctccaaaagaacagtgtaaaatgcagtaatagagccacctgcctttaactttcctggcctttcaagaagtctgggtaagctatcaaaaaccgaaaccggatagcctcttctggcaggtgattctccagcggctaaggccacatcacgtaatgctctggcataccttgttaatgaatcaataaaaagcgctactttatgtccttcagtcctaaaaaattcggctatagcagtggctatatatgcagcattacaacgatcaaccgaagagtaatccgaagttgcatatactaaaacacacctgttttttttctcagagtttttcaaataatcaaccgtttctgtaacctctcgacctcgctcaccaattaacccaataacatatatatcagcaccactatgttcaatgagcatattcatgagaaaagttttgccacaaccagctgacgcaaaaatccccattcgctgtccttcaccacacgtgagtaaagaatcaataaccttaatacctgttaaaaaaggcttctcaattgcagccctttcactatatagcggaggagcattatctacaggtcgataaagaatttcactgttatccgtaacagcaaacttatcagtaacctcccctaaaggattgactactgctcccaataatccacggccgacttgcgtatgaagaaactgggcagtgggcttaatcaacgtttgccgtgaaagtccacgagaatttccaatcaagcttaatattgttttttcatcatgaaatcctacaacctgagctcttgcaacaatttcattactttcaattccagcctgaatgttacaaatctcaccaatcgaaacatcgctaagacttgtttccaagattggccccgagattctattaggaaaagataattgagtgagcaattttgtatagctcattgattataagacctcatttaagatttccatcctctgataaaactcatgcagaatctctgcaaaaacaatcccatcgtgaacgtagtcatctttaataacaactcttaactgtaagtactcatcagaacgatgaagctctaccagctcattaatgctataactaaagttcattaacattaaatttaatatgttataagcagatgactgaagttttacatcacttggtgcatcaaaattagcccataacataacctgctcattgacaagagctatattaatagcaggcatactatccaaagaaatggtaatcgctgaatggctgtctaaatctgtaataattgatggaggacaaccaatcgtgaaaagactatctctaactaattgaactaaattaatgttactcatatttaaacctcactaaatagtctttaatacattaatggtatatgcttcatcaatctcagcatacgatataacgagtatactcttaaatctgttatctataagtcttttaacaaacctacgtatatctactgataccaaaagaacgaaatttttttttgcatttctcaattctctcaaagcatgtgctaaagtttccattacctcatccgaaacctctatatccatattcaagaaagagccaccagaggtttgccttatcccttttcttattgcatcctcaatatatccggaaagcatcacaactttaatctcaccagaaacagctattttactacaaatatacctagaaagtgatgcacggacatgttcaactaatgttatgacatctttttctctgggagcccaaagcgccaaagactccataataagttttaaattgcgaacagaaatattttctccaagcaatctttgtaatacctcagaaattctctgtatagtcacatgtcggaagacttcctttaatagatcaggataccgattttcaaactgatctaacatattttttgtttcttgtataccaaaaatctcatttatgttgtttaataaagcttgtgacaactggtgataaaattcatcttgagcacttttaattttagcatcaatattagttagattttctgtatatgaaacgtctacccaagaaataacacgttcattatatgaagttgaaacaacaggaatacctagagcatctatatctccattttcatctgtaatacacactttgtcaaaatatatattaaaactatcggcacgcacctcatttattaacaaaacaatatcatcgaccttaagctcattgcttgttctatataaaatagttggaagcctgacaccataatcaataaagaactgacttcttatcctttcaataagaccttccatatcatttgcatttatcttattttcggcaaataataaaattaaaggaacggtttctgaactaatagcatccagattttctatcattgccaaagatgagtcatggctattatcaatatcaaatgtaccagtatagccactagaatcagactcagacagacttttttctttttctacgacctttttataatagaataaagccgtcaaagtaactgctatcaggaaaaaaacaaaaaacggaaaacctggcaacatccctatcgccaaggcaagagctgatgtaacaatgagaacaaaaggattcccaaatatctgggacattatgttacgtcccatattatcgctatcaccattaactcttgtaacgataaatcctgcacttatagaaattaacagagcaggaatctgagagaccaatccgtcaccaatagttagtatggtataagtagataatgcaccagatagggacattccatgttgactcataccaacagaaatacctcctattaaattgacaaaaataataatgatgccagcaattgcatcacctttaatgaacttcattgctccatcaaatgaaccatacaattgactctcacgttccagaatactgcgtctttctttagctcctgcggcatcaataattccagcctttaaatccgcatcaatgctcatttgtttccccggcataccgtcaagagaaaagcgagcagctacctccgcaaccctttcagatccttttgttataacaatgaactgcacaacagttactattgaaaaaataacaaaacctacagccagactatcaccaatgacaaactggccaaatgtagtaataattttacccgcatccgcatcaactaaaattagtcgactagtacttattgataaagcgagacgaaataatgtcgttatcaacaaaaccgaagggaatgtagaaaaactcaatattctttcaatataaaatgatcccataaaaacgagaatagcaagtacaatattaagccctattaaaaaatcaacaaggtatgtaggtaatggtatgatcaacatagcaatgatcataaccataagcactaatataattaattcaggcttagtacttacttgcttaagaaaagactggatcactttcatctcctgttatctagaaagctctttcttgtatgcacttgtgacaagctctcgtagaatattgatcgcaatttctctttcactttcacttttaaatatatcatgaggactttcattaacagaagatagatatgtttgatacactacagaatcctcatgactggctcgtgatacttctataacagaagtaagaatttgctcactttcacttggatattgaaaaatatataataaagttagcaatagctgctctgcacttaaactgccatcttttagcaagcttgaggattccagtttcttcataaaaattacgtctgaagttctaatcatcttaattgcaattaatttagataacacaaaaccaaactcataagcattacagctcggtttctcagaatccatgtctacaatcagtgactgctctacaaaattcacaattgtatgtctgtgattaaaaccaaaattaccaagccattctatatactgatctgttgttgatatgttccccatgataaaaccacgataacatgcacgcaaaagctgtggttcaagtttcattttgttgccaaataatttagcctgtatagctgaatttattccagcattaacacttttttcactacctgatattatctcattaataattttagttaatgattcgacttgttcagcagtaagatctttttcattcaaaagttcccttaatgctaagacttgatcacttggatctttaaaatatctctttagtctgtctataaaatcccgatctagaggaaggttatcttttaacgttctctttaaatcaaaaatcttgtgattaatttcatcttcttccccatctaaaatacgttcctgactatcactatttgttcctttcagtttctcaaggtctcttctgtttataaatgacgaaagagcggaagccatctcatcagatgagtcaatatacttgctattagccaagccagaatcaagctctgcatcagcagtctcatctccatcatctgaatttgtcattgcattcaacttatcaatgaatgcagagctaaaaactcctgtatttttaacatcaagcatacatcacctactttatagttagtaatttaagtatgaaaccaatgatttttcgtcttccagcaaagtagtttcagatgggattatttgtgtagtcttttgtatttctctttcagatatgaggcttttatattctgcagtattataataacttgactcctttatctcacggggttgaatcagaaaaacccttacaatattactgatattacttgttttatacttgaacacattaccgattacaggaatagaggagagaaaaggaatagatactatttcgttagagttcgtttcatgcgtataaccacctatcaacaaacttttgccttgtggaactctcgctattgtactgattttggtccttccaacttctggaaggacagatgtattttcattattataattgtaatttgactgactatttcctgttccatcctctatcgttaaagacatctcaatttgtccccgagatgaaaagcgaggtataacatttatcaatgttccatacgtaacatgctccaacgaagaattgcgctcaccaactaatgacacataaaaagtcctgttattatcaaatatagccggaatattctcttgtgtcaaaatcaccggccttgaaactacatttgctttctttttctgatttaatgccataactgaagcaataaacttattgccgtccaaagtgcttatcgatgctgatgagctcatattaaatgacgcaccaaacgaatcaccaaatgaagccgttccttgccaattaacaccaaggttatttaattcagatttatcaatatcgattatccataatgataattctatatgtcgtttggctacatctaactgagtgataatatcgcgaataatctgtatttgctgatcattacctttgactaatatagaattggtttctggatatgcaataagtgatacatcctctaatatagaactattggtaactgaactaaatgaaaagtcattactatcctcagatactttctgtgtaatattaaagggtggcatcggatcattttgggcttgtctgttactcagagccttaccattatttaataaacgttctacaacagtagccacccctgggatgacaatatcctcccctctcatattataagttctgtcactaacaaatgtattttttaattttataaccccaaaattaactttatctgtcccaatagaagaaacctgcttatcaagcaaagttgctgtattagctaccagttcaaccaatgccggaggaccagatatatagaatgttttatcactaatatttcctcttataggatatctgtgatcataaaggttagcatccttcagatactgaataagataattaagtgatatattctccagcaaaataactttgcttattaattctccagagtcatatatgtataatgcatttccatctttataccatatgagtcccacaagtaatgtcagtttttcaagcatttcttctggattagataaatcaaactccccagatattctcttttttgcagcttgtttgctaacaacaataggataatttaataacgcagaaaaacggtcaaaaaaagatcctaccgtatcactttgagcaacatatttcgctatgtcattttgttctaataaatgagagtcaatattattagcattaactatcaatggtaacaatacaatcaagagagtcaaagatttaatattaaattttttcatttatttttttcactaaaaaagtaatattcgataattctcttgcagaaaagcctagtcgtgtttttatctcatttgaaaaatgggatgttgatgcatacccattattcatggcagcagaagtgattgtttgattgtgcaaaaatacatctaataaaccatttactaaccgccaagtgttcagttgttctttgacttttgcccctaaagcttttctacataaagatctaaaataagcctccgacacaccataatgctctgtcagactctttattcttacctccttttctttctcaatagttctcagaagataaagaaccaaatgatatgactcagtcattcgtatcatagccagtgcattacttacattctggtcagatgaaaagatgaatcttaacattaacttatcaagcatcgattcggtattacaactatcccgaagatcattctcatgatagaaatgaatacatttttcagcatggctatgctctactagagcaccactgaaatagtcccaaaaagcaagaaattttgcacaactattaatgttaaagcgataatgatagttcacattatcactgaattttatattttgatttcggacaaaaaaaaacccttccatgctggagccagctcgattatgttacgttctttatcttttacaagctcaacgttaactggttcatgtccactagagtaaatgtaaaacatattacttgtatttagacctttatatttactcacaatacaattatatccctcgaattaattttaatttaacttccccatccttctctataaggatggtatcatttatcttattaccttttaaaacaaccccgtctttcagcatagaaaatagtttatctatagctatcttttgtgcatccatgaagcacccatctacttccttaacattaaagttgcccatgaatttacctgcattaaaactataaaccgccttaccactaatacatttagagtaaacatttatacttttattggttgtattaaacgaccataataaatcatttggaatagaaaaataatccttacttacaggaacaatatgccattctttttcagaattagatgatgactttaaagcacacccactcagtgttaatagcaatatacttaaaataaatattttcaacttattactaccatgtcgaatcatctgcctctctccctgcttcaaagacactaattaatgaatcgaaggttgtagtcttaatatagaaatcaatattgttatatatggacaatgatgcataaaatccctcatgcaaatcgacatattgatttgcatctatgagtttattaagaagtgaacttttttgtgcagctgtaagataaggaaaacgtgaattgaataattgttcaatcttattgtgcagttcatcattatctatggaatttaattcactctgtgagaaatattgcattatttcatagattatatcctcatcaaatgattgcgaagatgataatctctcatttaaattaacaaattcttcactatttaatctttgctgcagagcattgcttgcatttatttgattaatcattaacaggattctcttttaacaagtcttctgcaatttgtctcagtttcttatgtgctaactctttgaaatattttttatcattttttgttaatttttccgcaagttctattttaacacctgatattacatcctgaggactaaactccactacttgattaccactacagaatatataattattcccttcagaatattttaaaataatcttgacatctgagcgatatttatggagatcaagttctaactttttcctcagtgctaaattacacttgggcaaaaccacttttaattctgttactgtattacgcaacgaactaagccactgctccaataaagtgctaatgatcctctcatcatgtaaattttctgaaagcaaactagttatatagtcctcaatatttcttctgttttcattttgctttaacttccattcacaaatatagtcaataatatgctcaaaagcagtctgtataccaatttgataaccatcacatacagcatctatacgcagtgattcagcttttttcgtagcatcctttataacacaaattgctttcttcttactctcagtgtctctatcttttatggttttacataattccttttcaattctctttataacaacaccatcataggcatgatgtctggataccggtaaagtgcctttttgcatattgcataccttcatttaatcgggacaaatttctttctacaggcgtataatttccctccaaagcaattggattaaataataagtttattctttcatatataccaacagatatattttccttccacgcaataagatgccaaaaaccacttcgaatagcggcctccccttgcttactgattttgcccccataataactaataaccatctcactatgattaattaaccgagactcttgactaccgataaaataagcaacagctggaagtaaattccagttggcaacaaacatagcggttaccccgcttaaaggttcaatatcgcaatttagatcatatttattaattatgagattattaatctcacttctgattacgccattttctaacagttcagaaggcaaattcaaccgatttctatttatatagaacgctggatcaaaaattattgaaagatattttttataaattccatccattcttatcatattttgtttctcttgaaccaccctgttttgaatgcccataccaaaagaatgacaactagtacagccatcccgagaaataaatatattacttcatttgttaaaaattccgatttaatttccttaacaggttgtacatttgtataaacatattcttctttcggagttaatatgacagatatattttcatacttaacatcagaaaaggtgtttttcaaaaatcgcttaatattactaactaatagttcagactctttcggtgagtcatatatagcgattactgatatatgcatcggttttgaagatatatttttttcttcaagatcatagctaacatgtatttttgccgaaataacaccaccaatagaaactaaagactgttccagccgttgctcaatagcactatataaacgggccttttcagctcttggagaagacactaatgaatctgtaggaaacatttgtgagatatcaactctctccggatttggcaaatcgtacatgcgcatcaaatcaactgccgatgcaaatgtccccttttcgacttgtaccgagatcccctgtttacctccatcaacttttctagcagtaatattatggcgttctagcacagatattatttcatttgcctgtctttgagataaattagaaattaactcttcacgttgctcacatccaatcaacatcagcaacaagaataaaataaaacctttatacctaatcatataaattaagactttaataaagtttcaacagccgaaactgtttttcttgctaacgtgccagctaatgatactcctattgaataatttgagagcgtcgtctgtaacttggccaaagattctgggtttaatgattctggattgcttaccatctccattatttgtgatacttgaatatctgtatccatcttaatatcactatatttagccgacacgacatcttccagacttgatatctcttgagattgaaaatcactggctttgataatatcaacctgattgactggataaatgtaattcatgaatgctccctattatctgaagttttgaataattgcagcatcaacatccttaatcactttcactgtattggattgcgcgttcctatataatgtatattcagataatttactttggtattcagccagcaactgtggattcgaaggatttttagctaatttatctagtgccaatgttagttcaccttgtaatgtttgagttccatcatcaaaagtttcagataatgaactcagtgtccaatcatcattcggtactgtaacactcattttatcctcacttatttttatctaaaaaaaaccagtgtttatcattcaacattacataactgtctttgctgttaagatatgatttacctttaaaatcatcatctatcaaaagtacagaaaactcaatatactgatctccatatatatttttatgttcagaaatcagtctattaatgagctcaagcttttcatctgtcagttcttctctgacagaatatgtaactttattattttcacaaatctctttatactggacatttacctttgtgaaaatacctttagcatccccccttgcattatgatcgctaagatagttgaatttaatatttttgatatacggaaattcaacaatcagagcatttttcaatttatcaagttcggtatcttttgaactatttctttctttacttaaaaacaccagtaattccgctttatcactcactaaattcaacacatacaaattgataaatgggaatcgttgattgatatattgttgaattctatttatttctttgtttgacaccagaatatatctgcatttggtaaatccggttttatttaaagctacagatgcccataccgcatcattttgtgtttcggcaagaatataattgcactgtccagtgttgactattccatatctcttatcatctataatttcagcaatttcttttacttcatctttctttaaagagagggaaaaaattataattaacacaacaataacagcaaagaaaaaaaacaccgagtggttcttatacattccgtttagtatatggcctctcgatttatcctccctcatatttttaaggtgaaaagaaatcccgtcatattcaaatgaattaaattgtgaattgatatcaatttgctcacctttttcatcactaaattgacagatattatctttgcttatggtcaaataaattataccatccgatttactatctgttacaggaataatattttctgggaaattgtcgtttccaaactcaagtgtttctatcgtccgtccgagaacaattaagttattcccataatataacggaaactcatccccgacaccgtttgtgagtttcactaataacctgaatggtgcaagatttgagttctttgcctcagacataaatcctcttatatccttcgattattctgcttgctctctgctgctatccgcaaatagcgtctatagatatcctctgcgtactttaatctttcttttttctttttcggagacgttcctgcattataggctccaacagcctcccaccctcttccatacatcatcataaactcatttaataactttgctgcataatgtacagaaagacatggatgactaattagcatttcctcagaatatcccatttctctaagtcttttagaatgaaagtcatttatttgcattataccataatctttacttccattgttattaacattgactgcaaatttattaaatccggattctttttccgcaatcgcttttaacaaacttgatggaatattgtacctttcaccagccttatcccaacaatcagctctccctaaatatgggataaaacacagcaaaataaatacgaaacgggacattaataccccttcattcttcgcgcaaattcatccttcagccaacgcacacgcgaaatataatattctgtattaacatatacatcctcgaattcatttatcccggtaagatctaatcgcgcaattagatttccgccttcatcatcagtagctaagcatatcttttctgagtaatttaggctcaacgcatatataagatcatttatattttcaggcaatgcacaaaaagggcataataaatttattgaatctccttctttttcaatatatattagcacatcatcatcaagcatgattgcttgatcatcgatatcaattaaagggatacccaaggctcggcaaataacatctgctaaatcttccatatattcctcttatacaaatgacgaataccctttcaccatattccatatttttgagtcccctattctttcagaataagatagctctaacgaagatagcggcaattttttcatgactttatttccgggcacaccagtattcatctcttggatttccatattaccactattcattagaatagtagaaaataatcttttctcttctgaggataatttactatttaattgagaaaactgacctgtttcatgttttcttattatttctcttttaatttcagcatcctgcatacctgttctgtccttcccactcttacaattccagcaaggtacagcacctatagtataagccaatagagtcattctttgtgacaatttatacggctctccattatcatttttttgtaacttcttgtttataatctcttttatctggttagccagatatatcacatcattcttacatggtggatttttttctattgcttctgcagcccaacctccaatcacaccatttttgaggaaattatcccccaacaaagaacatattgattcatcattaagcttgtcaacattcctccagcctaagcccatttttagcgctaattcatttacaccaaagttaaatgtcaccacttttagattaacgcttacttctttcagaagaccatcactattccgaattaataatttcgttggctccccccttttagagtttaacccttttaatgcgttcacctgatcttttaacatactctcctctcccccggttaaactggtcggcgtcagaagagaagtcgaaacaatctttaaatctactgttttaccagacaaagcctgtgataataactctggtctgctatataatgctgcgcttactaactcctcagctttattacgagcagcaacagctctttcagatgagttttttttcaatccataggctgatataactccatgtctgatacccgaaaaaatttccttcccctcgtcatccaccacttttgaaagccacatattcgcgatatgatcactctctctcgtagaagcacaacaaattccttttccattatactctttgacaaatatatttttattccctatcttcatatgagaggcaggtgttaattgaaaaccataatttttcccatgatgactaatattcttgttgaccggtccccaattcttattatttaaataatcagtaaatatttgttttattttcgtttgcaattcctttttatttttaatgcctagctgtgctcttatctcttcacttattactttattaagctcattaatatgtcttttattaacttcaccaagtttataggtatctaatcgttccatcatggctttaatttttgacgtatcaccacctgccaactttattgcctcgtaaaaaacttcagctctgtcacgcgcaaacaaaaggctttttcctgttaagcttgtttggttctgtaaatataatcgattaagcgcttcgatccccttatttctttctccatttttaactctttgatatgaaatcacgcttacacctttgtgctttccggtttcttctgcgcctttataggaatctccgctttgaaatgaaacctgatgcaatcccaaattagttatgtgcatttaattatcctcagtcataaatggggcattgattgcagttttgtacctcgttttacaacaaaaaagatacttattcaacagggttctctctttttctataacactccgagcctgatatatcagacactttaacttgtgggatttcatgatgaaacgagcactacatatgctcacgaggtacatattatgtacaacaaaagaaaatttgattatgttaacaggctcaagtttctaaatttattaattgaatatattatatatcaaatacattttacatccccacaatcaccacgtaatggagagttaataaagtatgatcctcaaaattagcaatttcattgacgcaagcaatacaaaagggcctatacgcgttgaagatacagagcatggacctatattggtagcacagaaattcaacctaaaggatctctttttcagaacattaagcactattaatgcaaagataaattcacagatattaaatgaacaactaaaaaattatcgactagcgaatcaaaaatccttattgcttttcctcaagacattagctagcgaaaaatctgcagaaagtgcgtttgccgcctatgaagcagtcaaaaattcaattcaacattcttttacaggtaaagacatcaaattaatgttgaataccgcagagcgttttcatggcatcggtacagccaaaaatcttgaaagacatttagtttttcgttgctggggaaatagaggcataacccatttagggcataccagtatcagtataaaaaacaatttactacaggaaccaactcatacctatctcagctggtatcccggcgggaatgttacaaaagatacagaaataaactacctttttgaaaaacgctcaggttacagtgtcgatacctataaacaagacaaactaaatatgatttcagaccaaacagccgaaagacttgatgcaggccaagaggttagaaacttattaaattcaaaacaagatcaaaataacaacaaaaaaatattttttcccagagctaatcaaaaaaaagacccctatggttattggggggtcagtgctgataaagtatacattccattatcaggtgataataaaacaaaggatggtaaaatcagctataacctttttggccttgatgaaacaaacatgtcaaaatttatatgccaaaagaaagcagatgctttcagacaattggcaaattataaattaatcagtaaatctgaaaactgtgctggtatggcacttaatgttcttaaagcaggaaattctgaaatatactttccactccctgacgttaaattggttgctacacctaacaatgtatacgcgtatgcaaataaagttaggcaacgcattgaaagtctcaatcagtcttataacgagataatgaagtatattgaatctgattttgatctttccagattgactcaattacgacgtagctatcttaaaagtttcaataaaattaaccttatccacaccccaaaaacattcaagcctttatcaatttcactttacaaacatcctactgaaaatgtatcttcagaagactttgatgcggtcatcaacgcttgccactcatacttagtaaaatcagcacccagcaatatgtctcgtgtattaaacgaattaaaaaccggagcaacagataaaaaagaagaaattatcgaaaagtcaataaaaactattgattattacaattctcttaaatcgccagatcttggaaccaaactttacattcacgacttattacaggtcaataagcttttattgaataactctcattctaatatatagaaggccatagaaatgtgtcgcaaactatatgataaactttatgaaataacaggtgcaaagcttgattttaacgataaaaatcaagcctttatattgctcgaagaacaaatacctgtttgtataacagataatgatgaatacatctttttgaccggacttcttaatgaacatgaactttttacagaaaatattattaatccagaacacatactcatccttaactactctttgagtagagattatggttcttctatctgtcttctccctgatactcatcaatgtgttttgaccaagaaacactacaaaaaatatctttcacctgatgaactcattgaatctttgtatgaatttttattttgtattaaactaaccattgcaaacatcacttcagaagtgaactaacatatagggggtatcatgcaaattctaaacaaaatacttccacaggttgaattcgcgattcctcgcccatcatttaattcgttaagttacaataagcttgtaaagaaaatattatctgtttttaacttaaaacaacgctttcctcagaagaattttggatgcccggtgaatataaataaaatcagggacaacgtcatagacaaaataaaagatagtaattcggggaatcaactattttgttggatgagccaagagcgaacctcttatgtctcttcaatgataaacagaagtatagatgaaatggccatacacaatggagttgttttgacctcagataacaaaaaaaacatatttgcagctattgaaaaaaaattcccggatataaaacttgatgaaaaatcagcacaaacatctatcagtcatacagcactaaacgagattgcctcatctggcctcagagcaaagattttaaaacgctatagcagtaacatggatttgtttaacacacaaatgaaggatcttacaaatctagtatccagttccgtctacgataaaatatttaatgaatcaacaaaagttttacaaatagaaataagcgcagaggttctaaaggccgtataccgtcaaagcaatacaaattaaatctgtttatattaaatcttatacttattaaagtaaagcagacaaaaaggagaccttatgtctttaaatatcactgaaaatgaaagcatctctactgcagtaattgatgcaattaactctggcgctacactgaaagatattaatgcaattcctgatgatatgatggatgacatttattcatatgcttatgacttttacaacaaaggaagaatagaggaagctgaagttttcttcagatttttatgtatatacgacttttacaatgtagactacattatgggactcgcagctatttatcagataaaagaacagttccaacaagcagcagacctttatgctgtcgcttttgcattaggaaaaaatgactatacaccggtattccatactggacaatgccagcttcggttgaaagcccccttgaaagccaaagagtgcttcgaactcgtaattcaacacagcaatgatgaaaaattaaaaataaaagcacaatcatacttggacgcaattcaggatatcaaggagtaattattatgcataatgtaagtaccacaaccactggtttgtctcttgccaaaatattggcttccactgagcttggagacaatactatccaggctgcaaatgatgcagctaacaaattattttctcttacaattgctgatcttactgctaacaaaaatattaatacaactaattcacactcaacttcaaatatattaatccctgaacttaaagcaccaaagtcattaaatgcaagttcccaactaacgcttttaattggaaaccttattcaaatactcggtgaaaaatctttaactgcattaacaaataaaattactgcttggaagtctcagcaacaggcaagacagcaaaaaaacctagaattctccgataaaattaatactcttctatctgaaactgaaggactaaccagagactatgaaaaacaaattaataaactaaaaaacgcagattctaaaataaaagacctagaaaataaaattaaccaaattcaaacaagattatccgaactcgacccagattcaccagaaaagaaaaaattaagccgggaagaaatacaactcactatcaaaaaagacgcagcagttaaagacaggacattgattgagcagaaaaccctgtcaattcatagcaaacttacagataaatcaatgcaactcgaaaaagaaatagactctttttctgcattttcaaacacagcatctgctgaacagctatcaacccagcagaaatcattaaccggacttgccagtgttactcaattgatggcaacctttattcaactagttggaaaaaataatgaagaatctttaaaaaatgatctggctctattccagtctctccaagaatcaagaaaaactgaaatggagagaaaatctgatgagtatgctgctgaagtacgtaaagcagaagaactcaacagagtaatgggttgtgttgggaaaatacttggggcacttttaactatcgttagtgttgttgcagcagctttttctggaggagcctctctagcactggcagctgttggtttagctcttatggttacggatgctatagtacaagcagcgaccggcaattccttcatggaacaagccctgaatccgatcatgaaagcagtcattgaacccttaatcaaactcctttcagatgcatttacaaaaatgctcgaaggcttgggcgtcgactcgaaaaaagccaaaatgattggctctattctgggggcaatcgcaggcgctcttgtcctggttgcagcagtagttctcgtagccactgttggtaaacaggcagcagcaaaacttgcagagaatattggcaaaataataggtaaaaccctcacagaccttataccaaagtttctcaagaatttttcttctcaactggacgatttaatcactaatgctgttgccagattaaataaatttcttggtgcagcgggtgatgaagtaatatccaaacaaattatttccacccatttaaaccaagcagttttattaggagaaagtgttaactctgccacacaagcgggaggaagtgtcgcttctgctgttttccagaacagcgcgtctacaaatctagcagacctgacattatcgaaatatcaagttgaacaactgtcaaaatatatcagtgaagcaatagaaaaattcggccaattgcaggaagtaattgcagatctattagcctcaatgtccaactctcaggctaatagaactgatgttgcaaaagcaattttgcaacaaactactgcttgatacaaataaggagaatgttatggaaattcaaaacacaaaatcaacccagattttatatacagatatatccacaaaacaaactcaaagttcttccgaaacacaaaaatcacaaaattatcagcagattgcagcgcatattccacttaatgtcggtaaaaatcccgtattaacaaccacattaaatgatgatcaacttttaaagttatcagagcaggttcagcatgattcagaaatcattgctcgccttactgacaaaaagatgaaagatctttcagagatgagtcacacccttactccagagaacactctggatatttccagtctttcttctaatgctgtttctttaattattagtgtagccgttctactttctgctctccgcactgcagaaactaaattgggctctcaattgtcattgattgcgttcgatgctacaaaatcagctgcagagaacattgttcggcaaggcctggcagccctatcatcaagcattactggagcagtcacacaagtaggtataacgggtatcggtgccaaaaaaacgcattcagggattagcgaccaaaaaggagccttaagaaagaaccttgccactgctcaatctcttgaaaaagagcttgcaggttctaaattagggttaaataaacaaatagatacaaatatcacctcaccacaaactaactctagcacaaaatttttaggtaaaaataaactggcgccagataatatatccctgtcaactgaacataaaacttctcttagttctcccgatatttctttgcaggataaaattgacacccagagaagaacttacgagctcaataccctttctgcgcagcaaaaacaaaatattggccgtgcaacaatggacacatcagccgttgctggtaatatatccacatcaggagggcgttatgcatctgctcttgaagaagaagaacaactaatcagtcaggccagcagtaaacaagcagaggaagcatcccaagtatctaaagaagcatcccaagcgacaaatcaattaatacaaaaattattgaatataattgacagcatcacccaatcaaggaattcgacagccagtcagattgctggtaacattcgagcttaatatatccaagagccataataatatatggctcttcctgtaaggaaataaccatgaatataacaactctgactaatagtatttccacctcatcattcagtccaaacaataccaacggttcatcaaccgaaacagttaattctgatataaaaacaacgaccagttctcatcctgtaagttcccttactatgctcaacgacacccttcataatatcagaacaacaaatcaggcattaaagaaagagctttcacaaaaaacgttgactaaaacatcgctagaagaaatagcattacattcatctcagattagcatggatgtaaataaatccgctcaactattggatattctttccaggaacgaatatccaattaataaagacgcaagagaattattacattcagccccgaaagaagccgagcttgatggagatcaaatgatatctcatagagaactgtgggctaaaattgcaaactccatcaatgatattaatgaacagtatctgaaagtatatgaacatgccgttagttcatatactcaaatgtatcaagattttagcgctgttctttccagtcttgccggctggatctctcccggaggtaacgacggaaactccgtgaaattacaagtcaactcgcttaaaaaagcattggaagaactcaagaaaaaatatgaagataaaccgctatatccagcaactaatactgttagtcagaaagaagcagataaatggcttacagaattaggtggaacaatcggcaaggtatctaaaaaaaacgggggatatgttgtcaatataaacatgaccccaatagacaatatgttaaaaagcttaaataatctaggtggaaatggcgaggttgtgctagataatgcaaaatatcaggcatggaatgccggattctctgccgaagatgaaacaatgaaaaataatcttcaaactttagttcaaaaatacagtaatgccaatagtatttttgataatttagtaaaggttttgagtagtacaataagctcatgtacagatacagataaactttttctccatttctgaggtgcgccatgcataatgtaaataatactcaagcgccaacattcttatataaggcaacttcaccatcatcaacagaatacagcgagttaaaaagcaaaatatccgatatccatagttcgcaaacttctctaaaaacaccagcatcagtgtctgaaaaagaaagctttgcaacgtcttttaatcagaaatgtcttgattttttattttcttcctcagggaaagaagatgtgttaagaagcatttattccaactcaatgaatgcgtatgccaaaagcgagattctcgaattttcaaatgttttgtactccttagtacatcaaaatggtcttaattttgaaaacgaaaagggacttcaaaaaattgtcgcacagtattcggaactaattataaaagataaattatcccaagattctgcctttggaccatggtcggcaaagaataagaaactccatcaattacgacaaaacattgagcacagacttgcactattagcacaacaacacacatctggtgaagctttatcattgggacaaaaactcctcaatactgaagtatcatcatttatcaagaataatattcttgctgaattaaagttaagtaatgaaactgtttcatctctcaaactagatgatttagttgacgcacaggcaaaacttgcctttgatagtttgcgcaatcaacgtaaaaatactattgatagtaaaggatttggtataggtaaactgtcaagagacttaaatacagtagccgtgtttcctgagctgttgagaaaagtccttaatgatattttagaagatataaaagattcgcatcctatccaagatggcctccctacacctcccgaagatatgccagatggcggaccaacccccggagccaatgagaaaacatcccaacctgtaattcactatcatataaataatgataatagaacttacgataatagagtttttgacaacagagtatatgacaatagctatcacgagaacccagaaaatgatgcacagtctcctacttctcagacaaacgatctattatcccgtaacggaaactcattactaaatccacaaagagcactagttcaaaaagtaacttccgttctaccacactctatatcagatactgtccagacatttgcaaataattcagctttagaaaaggttttcaaccatactccagataattcggatggaataggttcagacctgttaactacgagtagtcaagaaagatctgcaaataactctctttctcggggacacaggcctctgaacatacagaactcttcaaccaccccccctctccacccggaaggagtgacaagcagtaatgataactcatcagatacaactaaaagtagcgcttctctttctcatagagtagcttcgcaaatcaataaattcaactcaaacactgattcaaaagtacttcagactgattttttctcaagaaatggagacacatatttaacacgggaaacgatatttgaagcgtcaaaaaaagtaacaaactccctaagtaatcttatatctctcattggaactaaatcaggaacacaagaacgagagttacaggaaaaatcaaaggacattacaaaatccacaacagaacacagaataaacaacaaattaaaagttacagatgcaaatacaataaactacgtaacagaaaccaacgcagatacaattgataaaaatcatgcgatctatgaaaaggcaaaagaagtatctagcgccctcagcaaggtattgtcaaaaattgacgatacctctgcagaattacttacagatgatatatctgatttaaaaaataacaatgatattacagctgaaaacaataatatatataaagcagcaaaagatgtaaccacttccctatcaaaagtattaaagaatatcaataaggattaaaatatgataaaagaaaaaatattatcaatagtggccttttgctatggtatagcgtacagcaaactatctgaagaaactaaatttattgaagatctttctgccgattctctttctctgattgaaatgctggatatgattagttttgagttcaatctacgtatagatgaatctgcattagaacacattattactattggagatcttatttctgtagtcaaaaatagtacaaaatcaatatagtaattgataagcattttttcatctatggagctctcacatcagagctccacaagaatattattcttttatccaataaagataaattgcatcaatccagctattaaaatagtaatttttaagactaccgttgactatcatcaacagttacatacaatcttgctcacactgcatttaacttttgtcaatataaatcaatatgaataaacagggtgtgatatggtggatttgtgcaacgacttgttaagtataaaggaaggccaaaagaaagagtttacactccattctggtaataaagtttcctttatcaaagccaagattcctcataaaaggatccaagatttaaccttcgtcaaccaaaaaacgaatgtacgcgatcaagaatccctaacagaagaatcattagccgatatcataaaaactataaagctacaacaattcttccctgtaataggaagggagattgatggtagaattgaaattctggatggcactcgtagaagagcatctgcaatatatgcaggagcagatcttgaagttctatattcaaaagaatatatatctactcttgatgccagaaaactagcaaacgatatacaaacagcaaaagagcatagcatccgagaacttggtatcggtcttaattttctgaaagtatcagggatgtcctataaagacatagccaaaaaagagaatctgtctcgcgcgaaagtcactcgtgcctttcaggcagcaagcgttccacaggaaataatatctctatttccaatcgcgtcagaacttaactttaatgactacaagatattattcaattattataaaggacttgaaaaggctaatgaatctcttagttctacactaccaatattaaaggaagaaataaaagaccttgatacaaatttgcccccggacatatataaaaaagaaattttaaacatcataaagaaaagcaaaaacagaaagcaaaacccttcgctaaaagttgactcgttatttatttctaaagacaaacggacttacataaaaagaaaagagaataaaacaaacagaactctaatatttacattatctaaaataaacaaaacagttcagagagaaatagatgaagctattcgggatataatttctcgccatctatcgtcttcataaacatcgaacactgatgtaaactgcccaattcagataataaaaatcttcgttgaacttgactttcagccggtggggtaattccgtcggctatctgttattcaatccccacgcactgtgtttccttaaaattacggtgaaaactcataccctgctgaccacgagaacattatctggtgaaattagtaatattcatatcattgaaaatgaattgtcaactaagggatgcggcatgatggtcaaaattttatgtgaagatgagtgactggccgaaaatattcagcgtatttttaattgtttcaataaaaacagcgaggattagcagtcaacgcttttactactcaatttttttacatggtgataaacaaaaaatccaccattaacagacttcgttcacagtatcaaaaaagctttaagaatatattagcagatttctcaaatataattactgttttattctcctaatcttcccataaaatgaccgaattacattattttataattataaacaagtttgattttaaaatcatattaattattaacaagagatttatttttctaaccccaaaagttatgcttagtcaaagagtaacttataatatccatacccctcatagggcttacagtttaaattgcgggtataaaatacatctattttcaaataaaataaagcatactccaattacaacgagtgactaaaataaaaatatcatccaagtcaatcactgtttgctctgcaaaatatgtcagccttctcaaacttaccagcacttaaatatttaagcacacaatcattccttgagggagttcacttatttcaagagaaacatttcgtcttcccacacaactaatccgcccaatatagctaaatttgtactcagtaatatcatttgttatgccccccgcccccaaaatattcaagctccttcacagataaaaaacgacaatttacaaagcctcattagttataactatggaaatacctattttagtatatcctataaagttagaattacttcttcttgcacctaattccatagttgaaaaacaagaataattttctcctacagctggatccatatagcttccatcaggtctacacaacacccagtggaggccgacaggaactccaataaaggatacagcaacagcttccaaaactctttggttagaagataacacatctcgttgatgtactatattacatcccatgccaattaataagtcccttgcatcgggataaataaaactaattacttgcgaaaaaatgttactttcttcaacgacataagcattcaatccaagaagcctcgtagccatgactatcttatcaggcatagaataaccggcatccgcgatattatctttgtgtattctcggattataattaccactagttattaagtataaatcccgctcacaacgattatccaaatcaagagaactcttccttgtcatttcggacatagaccctttgtattgtggtattttatcaacccctaattcctttgcagcacagagtaaactggcagctccacaagaattgtcatacatctgcttcctcgccaaaaaacaaccttgttcgtttactctattctcatttaccctcacatcagtctgatgagatatcatatattctcttggtatagccccctgcaataaaaccccatataacataacacctgtatgaatacatccccgtttgcaaggcttccgttgttccgacatcgacgagtaattgcagcacagtattgcatccagcaatgcgaactactatgagaattagctgaccgggtccttattccccgaacgaactttaaatccaaaacctcatactcttttccgtcgtcatcacgtccgtcaccataacaacctttcagcatccacccttttattaagagttaatgtccatcagcatcagcaattaataccgcctgatactgacatatttttgtcgtatttacgtccataatcggttattgtgattcaggtcacaccagcttatttgctattgttacaataaccaccttggttgagagctgttttttcaactttgttatcgactcccccaactccttggtcagtaaggctgccgaccgagcacagtgaatagatagtatccgcaggtaccaccatgcttacttaatactcagaaggcgaagtgctctccaattaactctgccgcactatgcttataagctgcttttcaatgccgtctatcaacatgtccagttcattcaatctgttgtactggtaatccaacgcagtaatgatatatgcaggtattctttcatccactccagtaacgtttcatgcaaagcattaatttacgcgctacggaacttcaccagttgactgagcaaacgatgtgggaccagtactgcctgcagttcttctattgttacagcgatttctttaccaaacagctgaaccaccatcaagataaagatagcctgaacattcatcgcaccattttgttaccaatggcaaaagatttaacgaaccgtgcctgcatcaggcggactttttatccctgctttgttagctccctaaccttgtacagagagcctccataagctcccatatcaagcagacatggttcgcagttactgacatacaccagaaaactctgacagcagactcacttatcctccacttcactcatatccttattggtgagatgaacmwrmmwrmmmwrwwwwrmrmymwmmmmrmmmmmmrrwrwmwmwwwmmcctgtagagattctacaatctgtagcgttatgcatctaatgttaggctctatgatgtcagaaatctgcgagactmwacaccttctgcatttacctttatttatcaccatgctcctcagttaacggagaacgtgttcattgcattttgtcacttacatttcgcctttggtaccggggggatataggttccaacgttccgcatgagaacctgaaccggcttcacgccaccttgataccagatgccgcacaggcagtcaacaagtgtcacgaacggtgcaatagtgatccacacccaacgcctgaaatcagatccagggggtaatctgctctcctgattcaggagagcttatggtcacttttgagacagttatggaaattaaaatcctgcacaagcagggaatgagtagccgggcgattgccagagaactggggctctcccgcaataccgttaaacgttatttgcaggcaaaatctgagccgccaaaatatacgccgcgacctgctgttgcttcactcctggatgaataccgggattatattcgtcaacgcatcgccgatgctcatccttacaaaatcccggcaacggtaatcgctcgcgagatcagagaccagggatatcgtggcggaatgaccattctcagggcattcattcgttctctctcggttcctcaggagcaggagcctgccgttcggttcgaaactgaacccggacgacagatgcaggttgactggggcactatgcgtaatggtcgctcaccgcttcacgtgttcgttgctgttcccggatacagccgaatgttgtacatcgaattcactgacaatatgcgttatgacacgctggagacctgccatcgtaatgcgttccgcttctttggtggtgtgccgcgcgaagtgttgtatgacaatatgaaaactgtggttctgcaacgtgacgcatatcagaccggtcagcaccggttccatccttcgctgtggcagttcggcaaggagatgggcttctctccccgactgtgtcgccccttcagggcacagactaaaggtaaggtggaacggatggtgcagtacacccgtaacagtttttacatcccactaatgactcgcctgcgcccgatggggagcactgtcgatgttgaaacagccaaccgccacggtctgcgctggctgcacgatgtcgctaaccaacgaaagcatgaaacaatccaggcccgtccctgtgatcgctggctcgaagagcagcagtccatgctggcactgcctccggagaaaaaagagtatgacgtgcatcctggtgaaaatctggtgagcttcgacaaccccgttactttattcgtaccccttataatggggtgttagccagccagacccggcatgattactgcccccagtcgtccatgatccggggggtgatgtcaccgggtctggtggggcgctggtaaccgctaataggggtcaggtcaggcacttttgccgggaccgtctgtaacgtggatgccggtacctgctccccgtggttatctggttaacccatatacaagggagacagaatgaccgaatccagcgattacgaatccgtccaggtctttatcggcgttgatgtcggtaaagatacgcatcacgctgttgccattaatcgttcaggtaaacgcctgttcgataaagcattacccaacgacgaaaacaaactcaggtcgctaatatctgacctgaaacaacatggtcagatactgctggttgttgatcagccagctaccatcggtgcgttacctgtcgccgttgcccgctcagaaggagtccttgtcggatacctccctggactggccatgcgccgcatagccgacttacacgccggtgaagctaaaactgatgctcgtgacgctgccatcattgccgaagctgcccgtaccctgcctcacgcgctacgcacgctgaaactggctgacgagcaaatcgccgaactctccatgctctgcggcttcgatgatgatcttgccgcacagacaacgcaggccagcaaccgtatccgcggccttctgacccagatacatccggcactggagcgcgttctcggtccgagacttgagcacccggcggtactcgatcttctccagcgatatccctcaccagaaaaactcgcttcgctgggtgagaagaagctggcagcccagctctgcaaacttgcgcctcgtctgggtaaacgccttgcagcagacatagctcaggcactggccgaacaaaccgtcgtcgttcccggcacgaatgccgctgccgtagtactgccacgtctggcactccagctcatcacgctgcgtaagcaaagagacgaggtggcgcttgaggtagaacagcgagttcttgctcaccctctttacccggtcctgaccagtatgcccggagtcggtgtcaggaccgcagccagactcctcaccgaggtcgcctgccgcgccttcgcctctgccgcacatctcgctgcttatgctggccttgcgccggtaactcggcgatccggctcgtcaatacgcggtgagcatccctcgcgacggggtaataaagctctcaaacgggcgttgttcctgtcggccttcgccgcgctcagggatccgctctccagggcttactacacccgcaaaatgagtcagggaaaacgacacaatcaggcgcttatcgccctggcgagacgacgctgcgacgttctgttcgccatgatgcgcgacgggactttttataccccgcaggggtcataacatgcttgacaacttaataggggcaccccccaaagctgctgtctcttgatcagagctcctgatcaagagacttcatcaccaggtaaccctcaaccatatcctgaagtctgaccagccatcccacatgacgacccaaccgggacgacctgtgcgtttgtagactggccccctgaatctccagacaaccagtatcacttaaataagtgatagtcttaatactagtttttagactagtcattggagtacagatgattgatgtcttagggccggagaaacgcagacggcgtaccacacaggaaaagatcgcaattgttcagcagagctttgaaccggggatgacggtctccctcgttgcccggcaacatggtgtagcagccagccagttatttctctggcgtaagcaataccaggaaggaagtcttactgctgtcgccgccggagaacaggttgttcctgcctctgaacttgctgccgccatgaagcagattaaagaactccagcgcctgctcggcaagaaaacgatggaaaatgaactcctcaaagaagccgttgaatatggacgggcaaaaaagtggatagcgcacgcgcccttattgcccggggatggggagtaagcttagtcagccgttgtctccgggtgtcgcgtgcgcagttgcacgtcattctcagacgaaccgatgactggatggatggccgccgcagtcgtcacactgatgatacggatgtgcttctccgtatacaccatgttatcggagagctgcccacgtatggttatcgtcgggtatgggcgctgcttcgcagacaggcagaacttgatggtatgcctgcgatcaatgccaaacgtgtttaccggatcatgcgccagaatgcgctgttgcttgagcgaaaacctgctgtaccgccatcgaaacgggcacatacaggcagagtggccgtgaaagaaagcaatcagcgatggtgctctgacgggttcgagttctgctgtgataacggagagagactgcgtgtcacgttcgcgctggactgctgtgatcgtgaggcactgcactgggcggtgactaccggcggcttcaacagtgaaacagtacaggacgtcatgctgggagcggtggaacgccgcttcggcaacgatcttccgtcgtctccagtggagtggctgacggataatggttcatgctaccgggctaatgaaacacgccagttcgcccggatgttgggacttgaaccgaagaacacggcggtgcggagtccggagagtaacggaatagcagagagcttcgtgaaaacgataaagcgtgactacatcagtatcatgcccaaaccagacgggttaacggcagcaaagaaccttgcagaggcgttcgagcattataacgaatggcatccgcatagtgcgctgggttatcgctcgccacgggaatatctgcggcagcgggcttgtaatgggttaagtgataacagatgtctggaaatataggggcaaatccagttwkmwrwmmwrmymwmgmmmmargcccatttcagggtcggtgcctgcaccggaagcggctttccttccagcttaacccacagtagtttccactctgtcggcgtcagtattttctcacagctgtcattttgtgtttcttcactgacacctccctgccgcaggcccagcacccgtaccgctataaacgcctggataaccaccatgcgctcaaggttatcccgggtctgcattcgcagcgattccatgtaccaccacttttccacgccttgtggtattcctctgtcagccagcgtcgctcgtaatggctgacaatacgtcgtgcatcggcggcactcgccgcttttcctgacgtcagcagatgccagcaggcaccgtcctctgcctgctcccggcaacagacatacgtgagcgggagcgcctggccgctgttgtcgggattttttatgctgacttcgctgtaactgatgaacatccgggcctgacggtctgccagccgtatgcagccgaacagagacggtgcgatgctacctgttgctcattactgtagatccgcagttcatcatccagcgagatccgtattgataccagctggccacacaggctttccggcacactgtaacggttcccgccaacctcgatatagccatcccaggagacatgacggatatcgaagtccgtacccagcagcggctgcagatgctcctgttcctgcgtgaagcgctgttccggtgtctgcctgaactggcgaagttcccgcctgtcggcaacgtcagccatccactgcgcagcatgagagtttcctttgtggtgtaggaaccttaaggaaacaacatgttgggtggagcggacaatccaaatggtgaaggggggatgcggacgatttcctggactgctttatcattccagcccgcattgttggcggtacattttggggctgacggcacccagcgataattttatacgccgctcgttataccatctgatgtaagcatccacttgctgcatgaacttttctggcgtgatgcccgaccatttacgcccataatacatttccgttttcagtctgccgaaaaagccttcgcatgcagcattatcaggtgaacatcctttacgggacattgagcgaataagacctgctgcattcactctttccagccagcctggccagcgataatgcccacctctgtcactgtgtatcaccggtcgttcgccagcatttaacgtttcgacagcgctatccagcatagtgttgaccagttcagcatcagggcgtgtactgagagaccagctcacaacttttccatcgaagcagtccaccactggtgacagccagactttacctgcagggagctggaactccgtgatatctgtcagctatttctgattaggttgctccgccttaaaatctctggcgataaggttatccggagccggtccgatttctccgcaatatgagctgtagcgacgacgacggttacgactgacgacaagctgttcctccaccataagtctgcgtacaaccttttctgatagccgccccccctcatgcctgagcatcgcatgcaggcgacgatagccataacactggtaattactgttaaagatatctgtcagcatcttacgtatcgtggcgtatttatcaccggctttcagtgcagcccggtgataaaaataactgctacgggcaaggcccagaactgaggtgtactggcaatagcggacaccaccatttgttctttttctaagcagtcatctgataatattttatcctgaaggctgccggggagatattccccagacgagagtgacgacgctgacgattgtagaaaatctcaatgtattcccgtattactgagatggcctcatcccggctcttaaaacgatagtggctcaggctttcatttttcagcgttccccagaagctttccatcggagcgttgtcgtaacagttacctttacgcgacattgatgttttcagaccagactgctcctgtatgacccggtaatcgtatgcgcagwmmwrwrmmmmtmrawmrrtrwrrtrrattagmccggcaggtggrcgctggctcctgagcgcmataaacmrrrmwwwrmmyrwmmrmwmwwwyrwmmwrmrywmwmmmmwrrmrwmrmmrammmwwwmrmmmrwmwmmmmmwmwwtgacgccggcgagatacagccatccttcccgggtggcaacatacgttatatctgcgacccagacctgattaggtgctgtgggcgtaaagttctgattcagcagattcggcgtaaccggcagattgtggtcagagtttgtggtcgccctgaacttgcgtttctgcttacagtgcagcctcagctccttacgaagacgtgccagtctgtcacgaccgacgataattccgttatctgccagctccgtctggagccgccgggttccatatgtttcgcgagtgcggatatgtgccaccttaatctccagttttagccgctcatcactttgttttctgtctgaggggacatgctgtacccggtcgtaataaccgctcctggatacaccaggtamsmrmmammwmrmwwmmmmmrrrmmkwrwwrwmrmmmwwrwwcgactaacgcgtatttttcagcgactcctgggcaaaatacgctgttgctttttttaatatatctcgctcaagtcgtgcttcatttaaagccttacgcagttgcataacttcagattccagctcagccactgtgcgggagcctggtgtattgagcccttttcttgcggcggtaacccattgtcctaaagtgccttcaggaacagataatcgggaagcgccttcactgatcgaaagctgattttcaggaaccgttttgacggcttcggctttaaactccgtagagtaacgttgggtttttctgctcattattggctccttctgatgccattctatttcaggaaggagtgtccgttaaactcaggctacctcactcccgtaaagacgggagtcaagttcccgctccgacataactgcgtgcaatggattttgaaggaaccgtcagcccattccccggaggcaaacatgggctggcaaaagtgtagcggtattaggcacagctatttaggaaagagggaccatctccatctcattaaatggaccacgccagacaattcactgtcatgctgatgctgcactgtggggacagaatcactcatgatgacttcgataatcgacgacattattatttggccgagcttttaggtcatatctcaaatcgtttgcctaatattgctccattttttaataaaaaatcaatcatttttgaatctttgcttttcattgcattgtcaagcatagtgtccccactatttgctttttgaaattttttgtttacatcaaccaatccattattgataaaatattctagcactttatagttggcaccatctttactaagtaagtattcaacatcatacagattacaatatctattgttgttcattttctccatctctgcgacatcttgttttgtaaaattaaatttagatagtaaataaagtgctatatcagcttttttattgcctaaagaataagcaagggcctgatgagccatttttttatcagtcactttattgttaatctcttcataattagatgctttaatcgcatcctccaaagatatgtctctgagttttatttttttgaagttatctgtacttagcattcttggtatatgatactcaagctgaaacacaaagtttataattctatccagactaacaggatcaatatttttgttataacaaaactctctgaatccttgatctttactctttcttataaaatcaataagatgcaacccaattccaagcctcatatctttagtgttaaatattggaacatcagttttcccctgattaccatgcacatacctgctaacttcatccacaacctctttaaagttatctaagaaaaatgattggtgctcatggtaaaaaacaggtggaatagcattatcaaagtgatcggttaatgtcatatatccatatggagagtcatgatcaatgatatacgcattggcgccaaaatctacagtatgatgtgttttatttagtgggagtttttcttgatgattggaaatttcaaccccaaaaaaaacaaaatcgtggttagagatttctttaatatcctcagagtatgtattcttatgtgtttgtatattttcagactgaattttatcaaaggatttaatattaagaatattatttgacagattagagttactttgatgtctgaagttaaaattagaattaataataatagaaagtaagtgacgttcgtctgaagaaagaggatgaactttttgtattatctcagagatatagtgtctggacaccttatcgagatgctcaagtgaccgaaatgatgaccgtgctccagatggcgcttcaatagaaagatttagtgatttgaaactggtcccttgagaaaacattttgctataactttgcgctgctatagacttccttaaaaagtttttcacagtattagcacagtgatctaaatgtttttcacttatgccagaagaagatgagtttgttacgcgtaatatatttttttgtaatgcttttgttgctttattcggattcgtttttccatcaacaagatcaatattattctgaacattaatatgatttactgcttcaggtattttcatcccccccccaattatgtgcgtttttatctgtggttatttagataacattatctaactggacactttcttgtatgtattatagctaaatgaacagattttaatatgttgtatggttttcactctgaaatcccctgaatttaatcacgtttcctttactgaaggcaattttaaccgtagtatcaattattaacccttgggcttgatatccctctcctcttctgggcacgggggatgtcagattacttattacagaaaccaaaagaattgtaagcgtcatctcagtgtcgtctatttcaatgtctgatgattggcgatgatagagtcctcttataagtaagcgccccatcagcgacgtcttgtgaaaattgtcctgtctggcaacaatcgcgcccatctatattgatggacacgaacgatgaattcccagacaacaaaagatattccctgcttccgttcttatttgcctgatgccctgcgtttaagatttgaagataaactgaccatccgggccatcgctcagcgtctgggtctcagtcattccacaatacatacgctttttcagcgatttcttgcatccggtatcgcatggccattgcccgattcagtttcattcgctcaacttgacgccatcctttatgccaacagaaagaaggaattaacagagcctcaaatcagagaaggctcatggcgaaaagaacggcgaaccagctatagccgtgaatttaaggtccgtctggctaagcaggcattacagcctggtgctgttgttgcccggatcgccagagaacacgatatcaatgataacctgctgtttaaatggaaaagccagtacgaggacggcttactgagcgatgatgacatacaggaatgcatgcctgtcccggtggcactgactgatacgccggagccgaccagaccagttacaaatcccttctggcgtaagaaccacggcagcctggcggctgcaaatcggggtgtggcagaatatgagctttcagagtaaccatccagaatgagttcccatgtcacacaacctggagcaccagaaagtccacaccagaatggtgaaagaggttctgaaagccgtcgccagagccaataatcacccttatcagtccgtatttacagactttatcgccggtcatccctcctgcacggtctgtttctgggaaaccttccacaaaatgtacccggattcaccgtacgaatacgtcacgttctgccatacctgccgtcgttttgatttatacgaaacagaagcggagatgaaggcggatgacccgaagtggtggtgattttcagccccgaccgctgaaacgtctgttcacggccaaccagtgctggacatccttcctggatgcgggcggtctgcgcgatatcgaggttgaggctgtcacaaaaatgctggcctgcggcacgcggatactgggagtgaaggaatacaactgcgataaacctgagtgcccccacgtcagatacgtcactaactcatgcggcagccgtgcctgcccgtcctgcggaaaaaaggccacagacctgtggatagcgacacagctgaatcgtcttcctgactgcgactgggtacatctggtcttcaccctgccggacacgctgtggccggtgttcgaaagcaaccggtggctgctgaatgacgtgtgccgtctggcggtggagaatctgctgtatgccgcccggaaacgggggcaggaacccggtatcttctgcgccatccacacgtatggccgtcgtctcaactggcatccgcatgtacatgtgtctgtaacctgtggatgtctgaataagcatggtcagtggaaaaagctgagcttcctgaaagacgcgatgcgttcgcggtggatgtggaatatgcggcaacggcttctgaaagcgtggtcagaggggctggcgatgccggagtcgttgtcacatatcacgacggaatcacagtggagaagcctggtgctgaaagccggaggaaaatactggcatgtgtacatgtcgaaaaaaacggccggcgggaggaatacggcgcgctacctgggtcgttatctgaagaagccgccgatagcggcctcccggctggcacattacaacggaggggcgagcctgagcttccgttacctggaccacaaaacgggagaaacggcgacggaaacatgcacaaatggtgaaacagttcctgagtcgtgacccgttcgaatgcgtgctgtgtggcggccggatggtataccgccgggccattgcggggctgaatgtggaggggctgaagaaaaacgcgcgggatatcagtctgctgaggtatatgccggcctgacacaggctaagtgtgcccgggaatcggtgcaagtataaaaaacagcctgttrcnnnnnnnnnnnnnnnnnnnnnnnnnnnnnnnnnnnnnnnnnnnnnnnnnnnncagaaaggctcacgtggcataaggcgcggacaggggtatcgcttagtttttcaatttcctatacgtcaagctgtgcacgtccatccgatgccattttctcatttgactttcgggggattactgcctttataccgttatttttcagctcattgcgcaatgcgcgccctgaatacgctttatcagccagtaccgcatgtccacggcgtttcatgctgccgttctgacgctgaacgccaatcccgtccagaagtcgttgcgcgaactggctttcatgagcttgtccggggcttagcacgatatttaacgggaggccacttccgtctgttgccagatggattttggtgccaaaaccgccgcgagagcgacccggcccattatctccgtcgatatcgggatgtttttttgagcaccggcggcacatttcagcgcmmkrmwmwwmmyrmmmwmmmrmrmrgtrrcaracmartsaawmmmrmmrwwwrmmymmmgrrtrmykaytccarcttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgtgcagttgcccggcatccaacgccattcatggccatatcaatgattttctggtgcgtaccgggttgagaagcggtgtaagtgaactgcagttgccatgttttacggcagtgagagcagagatagcgctgatgtccggcagtgcttttgccgttacgcaccaccccgtcagtagctgaacaggagggacagctgatagaaacagaagccactggagcacctcaaaaacaccatcatacactaaatcagtaagttggcagcatcacctgcwkymmrwmrmrmmrrmmmmmwrwwrmmmmwmmwmywmmwmmsrmmwrmmwwwrmmmmwmrrwwmrmrmrrwwmwmmmmmrwtwtccmtgacccatatcgttcgggtaaatcgcgccatggtgcaccggaacataacacccagaacatgccattgatgattttacggtgctcagcccatgggcgtccggcccgtggtgtggcgggttcaggaggtaacaggggcttgatgatggtccatgcctcatcgggaaggtcgtagcaagccatagttcaatatgttgtgtaaacagacagttactataactcagatgattaagggacacagcctactattccgcgcagctatttagctctctttttgttgagcgcccatgattctttcatttattgttactaattttttatgttatcatcatgttatgagcatgccctgttctgcgtctctattttatctatcatattctgagcattaactacataacaaggcgcaagatatatatccaaaaacaaataatactgttgcaataattcctttcagtaaaggaagtgtgattaaactcaggggatttcagagtgaaaatcgtacaacatattaaaatatgttcgttataatacatacaagaaaatattaagttagataatattattctaaataactgcagataaaaacgcacataatgggggatgttttatgaaaatacctgaagcagtaaatcatattaatgttcagaacaatattgatcttgttgatggaaaaataaatccgaacaaagatacaaaagcattacaaaaaaacatatcatgcgtaacaaactcatcctcttctggcataagtgaaaaacatttagatcactgtgctgatactgtgaaaagctttttaaggaagtctatagcagcgcaaagttatagtaaaatgttctctcaagggaccagtttcaaatcattaaatctttctattgaagcgccatctggagcacggtcatcatttcggtcacttgagcacctcgataaggtgtccagacactatctttctgaaataatacaaaaaactcatcctctttcttcggatgaacgtcacttgctctctattattattaattctgattttaacttcagacatcaaagtaacgctaacctgtcaaataatactcttaatattaagtcctttgataaaattaagtctgagaatatacaaacatataaaaacacattctctgaggatattgaagaaatagctaaccacgattttgttttttttggggttgaaatttccaatcatcaagaaacactcccactaaataaaacacatcatactgtagattttggtgctaatgcgtatatcattgatcatgactctccatatggatatatgacattaaccgaccactttgataatgctattccacctgttttttaccatgagcaccaatcatttttcttagataactttaaagaggttgtggatgaagttagcaggtatgtgcatggtaatcagggaaaaactgatgtgccaatatttaatactaaagatatgaggcttggaattgggttgcatcttattgattttataagaaagagtaaagatcaaagattccgagagttttgttataataaaaacattgatcctgttagtctggatagaattataaactttgtgtttcagcttgagtatcatataccaagaatgctaagcacagataacttcaaaaagataagactcagagatatatctttggaggatgccattaaagcatccaattatgaagaaattaacaataaggtgactgataaaaaaatggctcatcaagctcttgcttattctctaggtaatgcaaaatctgatatggcactttatttactatctaaatttaattttacaaaacaagatgtcgcagagatggagaaaatgaacaacaatatgtattgtgagctgtatgatgttgagtatttacttagtgaagatagtgccaactataaagtgctagaatattttattaataatggattggttgatgtaaacaaaagatttcaaaaagcaaatagtggggacactatgcttgacaatgcaatgaaaagcaaagattcaaaaatgattgattttttattaaaaaatggagcggtatcaggcaaacgatttgggaggtgacctaaaatatatttaggttgtgccctgaatctgacgttgtcatttttgttatattgatcgatataggggatggtgcagaccgttcccctgcaacaacacattgcatgtggtgaacgccccgggaatcctggagactaggctgtgtcccttaattacgtaagcgttgataaaagagtcggatgcagcccagtttcaccatcgccaggtaatttctcgccgttttgtcgtaacgcgtggcgatgcggcgatattctttcagccgcccaaagcaccgttcaacgacattacgattgcagtacgcatcacgatcaagctgtgcacgtccatccgatgccattttctcatttgactttcgggggattactgcctttataccgttatttttcagctcgttgcgcaatgcgcgccctgaatacgctttatcagccagtaccgcatgtccacggcgtttcatgctgccgttctgacgctgaacgccaatcccgtccagaagtcgttgcgcgaactggctttcatgagcttgtccggggcttagcacgatatttaacgggaggccacttccgtctgttgccagatggattttggtgccaaaaccgccgcgagagcgacccggcccattatctccgtcgatatcgggatgtttttttgagcaccggcggcacatttcagcgcccggatattactgccatccagcgcggtggcagaccagtcaataaagccgtttgcatcaagtagcgaaagcaacctgttgaaaataatattaatcacgcctgactttgaccatcggttaaagcggttataaacagttttccatgacccatatcgttcgggtaaatcgcgccatggtgcaccggaacataacacccagaacatgccattgatgattttacggtgctcagcccatgggcgtccggcccgtggtgtggcgggttcaggaggtaacagaggcttgatgatggtccatgcctcatcgggaaggtcgtagcgagccatagttcacgtataggaaattgaaaaactaagcgatacccctgtccgcgccttatgccacgtgagcctttctgtttttccccaccactccggtgttttctctctctgcatggtcgattcttataccgcaacaggctgttttttatacttgcaccgattcccgggcacacttagcctgtgtcaggccggcatatacctcagcagactgatatcccgcgcgtttttcttcagcccctccacattcagccccgcaatggcccggcggtataccatccggccgccacacagcacgcattcgaacgggtcacgactcaggaactgtttcaccatttgtgcatnnttccgtcgccgtttctcccgttttgtggtccaggtaacggaagctcaggctcgcccctccgttgtaatgtgccagccgggaggccgctatcggcggcttcttcagataacgacccaggtagcgcgccgtattcctcccgccggccgtttttttcgacatgtacacatgccagtattttcctccggctttcagcaccaggcttctccactgtgattccgtcgtgatatgtgacaacgactccggcatcgccagcccctctgaccacgctttcagaagccgttgccgcatattccacatccaccgcgaacgcatcgcgtctttcaggaagctcagctttttccactgaccatgcttattcagacatccacaggttacagacacatgtacatgcggatgccagttgagacgacggccatacgtgtggatggcgcagaagataccgggttcctgcccccgtttccgggcggcatacagcagattctccaccgccagacggcacacgtcattcagcagccaccggttgctttcgaacaccggccacagcgtgtccggcagggtgaagaccagatgtacccagtcgcagtcaggaagacgattcagctgtgtcgctatccacaggtctgtggccttttttccgcaggacgggcaggcacggctgccgcatgagttagtgacgtatctgacgtgggggcactcaggtttatcgcagttgtattccttcactcccagtatccgcgtgccgcaggccagcatttttgtgacagcctcaacctcgatatcgcgcagaccgcccgcatccaggaaggacgtccarmmmwrrwwrrymrwrmmmrrmmrwwwmmrmrrkmkrrragtaaaagcggacaacatgaggacggattaacccccggtatcctgtagccgggtaagggctgctgtgtcgccacggcgctccaggatggaggccgcgaccatcgcaggtggtgggtccatgggtttgagcatcttcatccagtcccattcacgttgtgagagcttctcagcaccaacagagagcagaacggtatctatacttttgcgggtcaacataagaggatgagtccttaataaaacaggtcctcagagcatacatattctgcggagcgtgcggcaacaacaggacgaacaggggcggttttcaatttgcagccgtcaggctgccgtggttcaatatgttgtgtaaacagacagttactataactcagatgattaagggacacagcctggaacccaggatgttgcagggaagagaacttcattcaagcttcatcatgagaagccgatcagccaaaatggtggtgtctatgatatggataacatcagcgtggtaacacctaaaaggcatattgatattcaccgaggtgaaccgccccgggtttcctggagagtgttttatctgtgaactcaggctgccagatcatcgtttccgatggaagcataataagctttttctgcttctgccggaggagtatggcccagcctttccagcaatcgtcgattgttataccagtccacccacgtgagtgtggccagttccacttctgcacggtttttccagctcttacggtgtattaccttcgctttgtaaagaccattgatgctctccgccatcgcgttgtcatacgagtcgcctgtacttcctgttgatgccagtaatccggcttccttaagccgctgtgtgtaggccagcgatacatactgagaacctttatcactgtgatggaccgtgccggacggtcgacgggcccataacgcctgctccagtgcatccagcacgaatgtcgtttccatggacgatgagacccgccaccccacgatgtatccggcaaacacatcaatgatgaacgccacatagacgaagccctgccatgtgctgacgtaagtaaaatcagccacccacagctggtcaggtcgttctgccacgaactgacggtttacgcggtcgcctgcggcaacggctttccggctgatggtcgtacggacctttttaccccggagaacaccggcaagtcccataaccgccatgagacgtgccacagtgcatctggccactctgataccttcccgtaacaactgacgccagactttacgcacaccgtataccttgtgattttcatcgtatacgcgctgtatctctttcttcagccagtcatcgcgctgcgcacgggcactgcgtttatccggatgatgtcgctgttgctgacagtggtaatacgttgacggggcaatatgcagttcgctgcatagcggtccgaccccgtactgctcacgcagcttatccagcagtggcatcattttttccagaggcggtcaaactccgccttcgcaaaataagcggaagcctggcgaaggatatcgttactgcggcgcagttcacgattttcacgctccagctctttcagacgctgacgttcagcggtggtgagccctccatcaccgcccccggtatcccgctcatgctggcgaacccggacacgcagagtctccggcgtacagccaatctttggagcaatggaacaaattgtcgcccattgtgagtcatattcgccctgactttccagaaccatacggactgcccgttgacggacttcgggggaaaaacgagtatttttagtcatcctgtttacctctttctcaggragtttagtctccaggattcccggggcggttcaggccagtacctcgtcagtcaactgacggtaaatctgctgttcagtctcacgcatcacctgtgcaccggcttccctctccgcatccgcatcaccgctcagacctgatgctttcagccggtcagccaccctctgagagtactcattctccagcatctcatacttctgctcttctgcctgcgcccagcggtcagcttccgtacgcttcagtacagcatgccatggtccccagagggagaaccagtccgtaaattcattctcttcacggcttctgaccatggcttcggcagtgcggaggtcatttgctgtcactcccgacacgccatagaaacgcatttccttcacggcagtggagagctgaagtttctctgcgagcatggtctggaaggccaggtagacttctatctcatccacaaaatggagagttctgactttatcccgggcaatgtcctccagaatttcgaggcggaacatttccctgcccagggagagcagagcgccggtatcattatcgaaaaggccttctgatgcctgatggaccaggagggttttccggagattgttccatgtgagcgcgacacggtcctcacagctctcagtggcatcagcagcaacagcgaaagactgctgtcgaagctccgcagaggcactgagtttttccagccatgcagcgacctgttcacggaatccggaggtattgcgtgcagagacggtatcggaaaggcggtcaaggaacgcggaaaaggtgttggcatgctcttcatgttcaaaagcatgccatatctgtgatacatcagattgtttgttttccgggaaccatgctgtcacggcatcagccagggggcgatggagtgtattctgttgtccgtcactcatggagaagtaaatctgcgggccgtggtagtccggcgaagaggttaatctttgcagggattgcagaacgcgagtcgacaatggattccctgaaatgttaacattcgtacaaatgggcaatgtgattatattttcgggtaatgaagttagtaggttattggaaacatccagtgccaatagttcttgcggtaatctggataatctgcgtagttgattttcacctaccttcaaatattttaagcgctgaggaaattctggtagttcctgtagtctgttgccggaaacaaagagttcttgtagtctatgaggcaattcaggcaagacagtaaggcgattaccttcaatacgtaatattgttagcgttgatggaagctctggtaacgtctccagactattaaaacgtgccgagagtgattgtaaatatggtggtaatgatggcagacatgacaaatttcgattgaatgaaatattaagcttaatcagttgagatggaagtgtcgggagtatattcaagttgttaccatttacatgaagttctgtcaaaagcggagaattttcaggtagggagattaactcattgttggaaatattaagttcacgtatatgcaaagggagtggtggtaaagaacgtagtttcagtaagcttaaattgagaacaggctcccgggtatccagacatattttcaataatcgaaccgcctggatgcgattttctgttgttgttccttccctagcccagtcatcccagatgcggtggtaatctctggtacactgttcttctaccgataaacgagatatacatgataagccacaaagggaacgaaaaaaagaatgattgttgatcggtttcatttattaacccctcactattcatatcttggtctattaatgcaaataaaacgagaaagtctttaaaaaaacaatcctagagctttatcggcgttttcattccagctggaggaatatgttcctgtctttccagtaaccgtcgattgttatatcagtctacccatgttaatgtgaccagttctactttcatccagtttggattgatctttatcccctatattttcaaacaatttttatcatttaactcattactgtctcgatattcccatagcaaacgatctggttggttagcccctcaaaaaagtcagttgcacgccgtaaatccctgccggtattgagcgaggatttacgggttgttgttaaggtccgtaatcgggtcacarwwwwtccggagtcarwcmrgtctryggtttatgcttatgcgacgtgattatgaatggtgcagtcgtgagccgttttcagacaatcgcagggccagtacctcgtcagtcaactgacggtaaatctgctgttcagtctcacgcatcacctgtgcaccggcttccctctccgcatccgcatcaccgctcagacctgatgctttcagccggtcagccaccctctgagrgtactcattctccagcatctcatacttctgctcttctgcctgcgcccagcggtcagcttccgtacgcttcagtacagcatgccatggtccccagagggagaaccagtccgtaaattcattctcttcacggcttctgaccatggcttcggcagtgcggaggtcatttgctgtcactcccgacacgccatagaaacgcatttccttcacggcagtggagagctgaagtttctctgcgagcatggtctggaaggccaggtagacttctatctcatccacaaaatggagagttctgactttatcccgggcaatgtcctccagaatttcgaggcggaacatttccctgcccagggagagcagagcgccggtatcattatcgaaaaggccttctgatgcctgatggaccaggagggttttccggagattgttccatgtgagcgcgacacggtcctcacagctctcagtggcatcagcagcaacagcgaaagactgctgtcgaagctccgcagaggcactgagtttttccagccatgcagcgacctgttcacggaatccggaggtattgcgtgcagagacggtatcggaaaggcggtcaaggaacgcggaaaaggtgttggcatgctcttcatgttcaaaagcatgccatatctgtgatacatcagattgtttgttttccgggaaccatgctgtcacggcatcagccagggggcgatggagtgtattctgttgtccgtcactcatggagaagtaaatctgcgggccgtggtagtccggcgaagaggttaatctttgcagggattgcagaacgcgagtcgacaatggattccctgaaatattaatcctgtaacttgaatctaattgtgaaaaactctccggaagagtggttatctgattaaaatgaaaataatattccttttgtctgatattgtttctatcagaaaattgaggaaaagtaaccagattattgcgctgagcacagagaatttccagtttatccggtaagtcgggtaatgttgtcagcctgttataataaacatgtaaaaatttcaatttttcgggaagagccggtaagttatatagcctgttattttcaacaaataatgatgttaaatggtttggcaaaacaggaaggttttccagttgattagaacgaacacttaaagttgttaatgactcaggaagttctggtatcacagaaagcctgttattatcagcattaagttctttgagaaatgcaggcagctttggcaatgatgttaatttattattttctacaatcaaagcagaaatatgctttggtaattcgggaagagacgtcaaatttaggtctgataaatttaatactgcttcttgattctgaaagcactgggagagtctgttaaaagcaatgcctcgctcttccccgggagaagagttcttttcccattcagtcaggattctcagataatattcattactggttgagtttgagttaatagagggggagcaagaaactgatgagtgtgtattatttacagagaacataatgctaggtcaaccttatatttgtgagaataaatatttaaaattgtatatttatacaaatttcacttaccatttccgatattaagttttaatagtttccattttacacataacagaagttagttgtaatttatttattgaggttttctatggaattctttttttattgtgactgcagtcaataataacactgaattcaacattccagtgagcaaggaaatgtaatccgagtaagtatatttgttttacatcatattttagccaagctcatttattcctattatagaatgataaattaaaattactgtaaaaaaacgatacacatttaatacgagggaggccggcagaaaaacggcttgcggtaaggcaggagaaatgtgtgccgcgaatgaagtccttgtatgactggataaagctccagaggaaaactttgtcaaaccatgctgaaatggtaaaggcgttcaactatatcctgaatcactggaatgcactgaatgagttttgccgggtagtacttccaatcattagaatatgtgcttttcaatgaaggaaccgatgactttttcgtggatttccacggaacgcgagaagcagattgttttacgtgccagccgcttgatgcgggggcggagggtcaggttgttacgttcatacgttgcgtgaagattttgccggtcaggtgtttctcctccggcacttctctggcgtagctgccccagtcatcgctggtcaccatgccaccatgccgatacagaacggagtcaacagggctaacagttcccgacaggtctcatcattccgtggcccgcaagtgtaagccaacacaccgccagttttagtgttgtacgcataccagggccagtgttgttgcgccttgctgccaacgaagcgccactgctcatcaagttcacagataagggcaacatcagcgtgagcgactggcgcagatgttatccgccggggcgagagctttttaatgtccgtatgacggtattgatgccgattttcagtgttctggcggtataacagccccctgcgccgttatgtgccatttcaacgatctgctccttaacgccgggttttctggcttcataagagtaagtgagctggaaaacacgtttacatgagcgacaacggaaacgctcatgccgggaacaactacggccatgtcgataaacttcatcggaatgacagcgcggacaatgaacagtaacggtagtcatggaaaacctcaaataccagtagattaccagaaacttctatcgattgaagacattaccttttgccgcaataggtggggtaacattggtgaaaacgcattacgccgggtggcagttggaaaaaaattatcttttttcggctctgatcgtggtggagaaaatgcggcaataatctaaatagctgcgcttaataccgctacacttttgccagcccatgtttgcctccggggaatgggctgacggtttccataaaatggcgaacttttttcaacagttgccacattgagctgcactgatgattacgcgttattgtgtcgtgaagtgcctgccatagccgttcaacatgattcacccatggcgagtaaaccggctgataaatgaccctgaacttcgggttctccttcagccagctctgtgtttcccggcttttgtggataatgtagttgtccacgatcagcgtgatggttttcgcccgacggtatgtcgctttaagccgcttcagcaggctgatgaacagcgccgaacttttgctgttgccgcccacacagctgactttacctgtcccgctgtgcagcgctccggccagataatatttttcattctgtcccggcgtgaccacccgtttttgctgtccgcgcagttgccagtccgcaccgattttgggattaagatggatatccacttcatcttcataaaagaccggatgctctgcgctgcattcgtccagtgctttatggattgctgccatcttttcatctttatgcgggtcacggatacgcagagttggcgcagcccttcgccacacaatccccgcagacggcaaccagcggcgaacggttccggcatttaactggcaaccggttatctcattgatttttattgccagcagttctgtactccagcgtgaacgctggtagccaaagtcgccgggagaatgttttaccagctcacgtaacagtgtgcagatatgctcaaacggccagcgacgggcacgcccggcaggtaatgatttcagtccctcaacacccgactgcgtgaaccagttaatccagcgtccaacagaggaacgggcgcagcagagcgttctggcaacgtcgctgacacggtcgccccggtgcagcatcagcatggcagtcagtctgcgggcataatttttatcgtgtgttttatggatggctttctgcatcaggcgtcgttcgtcacgggaaattggtgctatgatcggcattgctcagtccggttggtgatttgttttgatttggcgattgatcagatcgcacaatccgggctaagttccctcaaagtgatctactattccgcgcagctatttaggywrwktcccwwmattacgtaagcgttgataaaagagtcggatgcagcccagtttcaccatcgccaggtaatttctcgccgttttgtcgtaacgcgtggcgatgcggcgatattctttcagccgcccaaagcaccgttcaacgacattacgattgcagtacgcatcacgatcaagctgtgcacgtccatccgatgccattttctcatttgactttcgggggattactgcctttataccgttatttttcagctcgttgcgcaatgcgcgccctgaatacgctttatcagccagtaccgcatgtccacggcgtttcatgctgccgttctgacgctgaacgccaatcccgtccagaagtcgttgcgcgaactggctttcatgagcttgtccggggcttagcacgatatttaacgggaggccacttccgtctgttgccagatggattttggtgccaaaaccgccgcgagagcgacccggcccattatctccgtcgatatcgggatgtttttttgagcaccggcggcacatttcagcgcccggatattactgccatccagcgcggtggcagaccagtcaataaagccgtttgcatcaagtagcgaaagcaacctgttgaaaataatattaatcacgcctgactttgaccatcggttaaagcggttataaacagttttccatgacccatatcgttcgggtaaatcgcgccatggtgcaccggaacataacacccagaacatgccattgatgattttacggtgctcagcccatgggcgtccggcccgtggtgtggcgggttcaggaagtaacaggggcttgatgattcgagcattttctgcccgcagtgccgataattcatcgctgtcagatttgtgaaccatgccctgctgtaaatctccgcatagattatgtctgtttgatctcagtcatccggtgtctggcgagaagacgcaccacggtggaagggtgaacccgaaacagacgggcggcatcagcacctgttttctgccccgatgtgacgagcgagacaatctctttttgttgctggggtgttaactttggtcgccatccaccaacccgtcctggcgtgccgctgcaagtccactctttgtccgttcacgaagcatggcgcgttcaaattcagcaaaggaaccaacgatttgcaccaaatactttttacgaggttcttttcgccagtcctgtaattccatactcaggtgtccatctatgtcagatgggcgtttaagttaccaattatcgcttacttacggggtgagtatcgtaaaaaagcggctctgcaagtcctcctaattggtgacattaccgaccatttttcttccttatgttttgagggtatgtctttaaatatttaaagaaaactttagaaaaatacgatggtgaactgtaacctaactcagatgatatgacagaaatgggagtatttgtccttcgtaatttcctgatagcttctgtaagctttattttatgtacaaatgtacttatagaaccgacctctttgtacattcttctttttaaagaagattcactcattaaaaccgcactggccaactcggaattactccagttacgggagtaatttgccatcacaattctggcgattttttcagacacagaaacaaatgatggtgcatgaagaataagcccaacatttttgttttttttcagaatacatgaaagcaaataagaactccgaactttatcgtaaatggcatctgtctccgtctttttattatcagtgcactgaggtagcctgagtttaacggacactccttcctgaaatagaatggcatcagaaggagctaataatgagcagaaaaacccaacgttactctaaagagttcaaagccgaagctgtcagaacggttcctgaaaatcaactttcgatcagtgaaggcgcttcccgattatctcttcctgaaggcactttaggacaatgggttaccgccgccagaaaagggctcggtactcctggttcccgcacggtggctgaactggaatctgaaattctgcaactgcgtaaggcgttaaatgaagctcgccttgagcgagatatattaaaaaaagcaacagcgtattttgcacaggagtcgctgaaaaatacgcgttaatcgaacaatggcgacaacaatttcccattgaagcgatgtgtcaggtatttggtgtatccaggagcggttattacaactgggtacagcatgaaccctcagacagaaaacaaagtgatgagcggctaaaactggagattaaggtggcacatatccgcactcgcgaaacatatggaacccggcggctccagacggagctggcagagaatggcatcatcgttggtcgtgaccgactggcacgtcttcgtaaggagctaaggctacgctgtaagcagaaacgcaagttcagagcgactacgaactcgaaccacaatctgccagttgcgccaaatctgctgaaccagacgttcgctcctacagcaccaaatcaggtctgggtggcggacctgacgtatgttgccacacaggagggatggttgtacctcgctggcatcaaagatgtttatacgtgcgaaattgtcggctacgccatgggagagcgcatgacaaaagagctgacaggtaaagccctgtttatggcgctcaggagccagcgcccacctgccgggctaatccaccactctgatcgaggttcacagtactgcgcatacgattaccgggtcatacaggagcagtctggtctgaaaacatcaatgtcgcgtaaagrwmmmwwrwkmwymrmmmwrmmrrcrtmmmrcttcagtgatccaggtccggtattccatacgatagtgtccacttcaaaatgatggacattatttttgtggaaccggaggtaacagaccagacggtttaaatgagccggttacctcaaattcagtgtatcaccacgagataatattcagctttttatattcttcataatttccagaaaatcacttccattttttttgtccaagtgttctgctatttcttcaggtgtaatattttcattttttattatttccatataagtttttattgcctcaaaattcctattttgaaaagctataattaatccagataccccatttgggccttctgctgccagtaattttgatgcctgatgtggagtaaggtctgcagtgtgaagtattttcccataagcaataattgtttctacacatccattatgcaaggctacaaacaatccagataaaccattagaatctttcgcttctaacagctctgcttttttatccaagttaagttttgcctcattcaagatatcaccataagctaagattgcatcagcctgtccattattcaatgctaacagaagtccgggaacattgtcatatctccttgaggccagcaaatttacaatatcctctgagttgaggagaggggggctaagaatgagctcaccgtatgcgcgtatagcatctgcatgtccattttgaagagcaaacattagtccgggtatctgtctgtgtacttcatcataataataagcggtcagcaaatttttcagcttattgtattcgatatttattgctctcttcttgagaagccttccgtatgttttgatagtttctgcatgcccattttgaagtgcttgggataaacccgatattcgtcctcccattccttccgcagttagtagttctaccgttttatcaaggttaagtccagaagtttccaggagttctccgtatgctaagattgtatctgaatgcccattttgtaaagctaaaagcaatcctgacaaaccatcgtcatttttggctgccaaaagtaattctaacgtattgtctgaaaggtcagatagttgtacgactcttgttactttttggattagctcagtcaatcccatacgcatcgcgtgataaataactttggggtgaagtaaattattaggccatctgattatggaggacatgcttgttggagtatgtctgtccacaaatatagacataccgtcggatataagaccataacatttctgatgtttttcatcaagaaaattatctacagtcagtttttttattttagctaagttaaatttgttaaactctgctcgtaaatgagttgctgtatggttgggatcataaagctgaatgacaaaatcccccccatttttagtttctttaatcttcaagcctaatgccatattgtgatttgatgtacttatttctagtgctctgcttttttcggaatgaaaaatcatttcattgaaaatataaaaaataatatctccccatgaaccgttttcaacaaaataaataatagcaggcacattaatagccttttcttcttcactcactgaaacataatttttaatttcatccggaaaagcaaagtgatgataatccacgtgaccactacttttgttaaactgtgctatccagtaggaagctaaatgtcgacagacaataagtcttccagagtgatcggtaacttggcaatttagatttaataaaaaactcctcttgtcagaaaaatatggcaattgttttgagtgcaatagagactggattgtctcaacagagacgttgtctttatttatcatattttgcaaacatattttccttgatggaattaaatttactgatggcatatgtatatattcctttcattgatgggttgtgacgcagacttagtgttgttgtactctttgccctttggatgattgatggcaaagattgatgtggtttgtccccgttactctgaaactcaggggggtatccggaacggccattccagttcaggggggctgctctatcgctgtaagcagtgcctgaagaccttccagctcgactaccgctacaacggggcgaaacccatcaaactttccaactattatcacattgagccatactgacaataaacaatatatcgatattttgtgcaaatactgccttttgctaaacgatattcaattttgattaaatatatttattgtcagatgttgcaccatatttcaataatagttttatcatttctgcattctcatattttattgcgttatccaacatacaatccccactattagtttttttgaactttgtgtttacatcaaccaatcccttattgataaaatactcaagtactttacagcttgaattatgagcgctaagtagatactcaatatcatacaactcatgctttatggaaataacatcttgtctagttatctcaaaattagataatatatataaggcgacatcctctttttgattagcaatcgaaagaaaaagagcctggagagcaatttttttgttagtgacctgcttgttaatttcttcgtaattagatgctgtaacagcctcctctaaggatatttctctaatcttaacttttttgaagttttctgtacttaccatcctaggtatatggtactctggctgaaaaacaaagtttatgattctatccagatccacaggggcaagattttttccatagcaaaactccttgaagctttggtcttcactttttctaataaagtcaatcaggtataatccgagccctaacttcatatctttagtattgaatattggtacatctataattcctttacttccatgtacgtatcgactaacttctttattaacctctgaaaatttatctaaaaatgattggtgctcatggtaaaaaacaggtggaatagcattatcaaagtgatcggttaatgtcatatatccatatggagagtcatgatcaatgatatacgcatttgcaccaaaatcaacagtgtgatgttttgtattcagggggtgttttttctgatggctagagatttcaacgccaaaaaacacaaagtcatgattagctattttttttatatcatcagcgtatgtgtttcgttttgtgcatattccttcagactgaattttattgaatgacttaatgtttaaaattttatcagacaaatcagaattactttgatgtctgaagattaaatcagaattaataattagcgaaagcaaattgagttcctctgcagaaagaggatgaagtttttcccttatttcagatatataatgcttcgataatctgtcaaggtgctcaatagccttaaatgaggctttagcgtctgatggtgcatcaattgctatatttaaagatttaaagttagagccttgagagaacattctactataactctgtgctgctatagttttcctaaagaaatctttaataatatttgcactatcttttaaatttttttcatctggcatagtttgttgtgcagcgtttcgcactgatgtcacttttggaaacaatgtatgtaatctgttatccatagaatctatattgcattgggtatttgctgagttcagtgtttctgatatattcataaaatgaaccttatatgaaaacagttttaattaatatctttccctacaagatatagcagggtatgaattacaatttttgagtatatatacaatatactgcatgtaattagataagggtgttatatacttttgatgttatgtgatgccagatatttcaaaatatctaaaatccgatgcttaagaactcaagttaatcttttttgtgaaattattttcataatttaataactatatcaattttattgaacagcaatgaattttactataaaaaaacgatactttctctgattcctcttatggagtgcttccagttagtggaatgtgtattctgcaatgaaatcctcgatgattttttcatgaagctcaactaagtgtgaaaaactgatgtttttttaatagaggttagtttggccatatataattattgtacttttgcagtttttttgttacttcccttatgagttcaaaatcgccttaatggagcagtcactgcaacccagagcctgtgtggcgtagatcgcccgtgaaaatggaatctgccgttcggtttttgcgcgaagtttttctgagcttttaccgaactacatacgctgcagtttcgcaaccagcgccttcagtcggttgatttcggaagcataagccgccatccgctgtgagagcaggcggttgtattcagccatctggtggatagtgtcctgttgcgtctgcaacagtgcccgcaggcgggcgttcacgtataggaaattgaaaaactaagcgatacccctgtccgcgccttatgccacgtgagcctttctgtttttccccaccactccggtgttttctctctctgcatggtcgattcttataccgcaacaggctgttttttatacttgcaccgattcccgggcacacttagcctgtgtcaggccggcatatacctcagcagactgatatcccgcgcgtttttcttcagcccctccacattcagccccgcaatggcccggcggtataccatccggccgccacacagcacgcattcgaacgggtcacgactcaggaactgtttcaccatttgtacatagcacactttcgccactggttccggtttatccatccccagagcacggtacacctgcggcagcttctctccacacacccggttggcaaggaacccgaagtacctcaccatcttaaaaaacttctccgggatgtgctgtttcagcctcgcgaccagctcacgctgtgtcagcgtttccgtcgccgtttctcccgttttgtggtccaggtaacggaagctcaggctcgcccctccgttgtaatgtgccagccgggaggccgctatcggcggcttcttcagataacgacccaggtagcgcgccgtattcctcccgccggccgtttttttcgacatgtacacatgccagtattttcctccggctttcagcaccaggcttctccactgtgattccgtcgtgatatgtgacaacgactccggcatcgccagcccctctgaccacgctttcagaagccgttgccgcatattccacatccaccgcgaacgcatcgcgtctttcaggaagctcagctttttccactgaccatgcttattcagacatccacaggttacagacacatgtacatgcggatgccagttgagacgacggccatacgtgtggatggcgcagaagataccgggttcctgcccccgtttccgggcggcatacagcagattctccaccgccagacggcacacgtcattcagcagccaccggttgctttcgaacaccggccacagcgtgtccggcagggtgaagaccagatgtacccagtcgcagtcaggaagacgattcagctgtgtcgctatccacaggtctgtggccttttttccgcaggacgggcaggcacggctgccgcatgagttagtgacgtatctgacgtgggggcactcaggtttatcgcagttgtattccttcactcccagtatccgcgtgccgcaggccagcatttttgtgacagcctcaacctcgatatcgcgcagaccgcccgcatccaggaaggacgtccagcactggttggctgtgaacaggcgtttcagcggtcggggctgaaaatcaccaccacttcgggtcatccgccttcatctccgcttctgtttcgtataaatcaaaacgacggcaggtatggcagaacgtgacgtattcgtacggtgaatccgggtacattttgtggaaggtttcccagaaacagaccgtgcaggagggatgaccggtgataaagtctgcaaatacggactgataagggtgattattggctctggcgacggctttcagaacctctttcaccattctggtgtggactttctggtgctccaggttgtgtgacatgggaactcattctggatggttactctgaaagctcatattctgccacaccccgatttgcagccgccaggctgccgtggttcaggtaaacgcctgttcgataaagcattacccaacgacgaaaacaaactcaggtcgctaatatctgacctgaaacaacatggtcagatactgctggttgttgatcagccagctaccatcggtgcgttacctgtcgccgttgcccgctcagaaggagtccttgtcggatacctccctggactggccatgcgccgcatagccgacttacacgccggtgaagctaaaactgatgctcgtgacgctgccatcattgccgaagctgcccgtaccctgcctcacgcgctacgcacgctgaaactggctgacgagcaaatcgccgaactctccatgctctgcggcttcgatgatgatcttgccgcacagacaacgcaggccagcaaccgtatccgcggccttctgacccagatacatccggcactggagcgcgttctcggtccgagacttgagcacccggcggtactcgatcttctccagcgatatccctcaccagaaaaactcgcttcgctgggcttcgctgggtgagaagaagctggcagcccagctctgcaaacttgcgcctcgtctgggtaaacgccttgcagcagacatagctcaggcactggccgaacaaaccgtcgtcgttcccggcacgaatgccgctgccgtagtactgccacgtctggcactccagctcatcacgctgcgtaagcaaagagacgaggtggcgcttgaggtagaacagcgagttcttgctcaccctctttacccggtcctgaccagtatgcccggagtcggtgtcaggaccgcagccagactcctcaccgaggtcgcctgccgcgccttcgcctctgccgcacatctcgctgcttatgctggccttgckccggtaactcggcgatccggctcgtcaatacgcggtgagcatccctcgcgacggggtaataaagctctcaaacgggcgttgttcctgtcggccttcgccgcgctcagggatccgctctccagggcttactacacccgcaaaatgagtcagggaaaacgacacaatcaggcgcttatcgccctggcgagacgacgctgcgacgttctgttcgccatgatgcgcgacgggactttttataccccgcaggggtcataacatgcttgacaacttaataggggcccccccctaagccgtctatgggaaggagtctgctgttattccagagaatggtaattcagtcatatatttctccatctatgacttgtcaataccatcctgatatttgtgggtcagcaatatattctactaaaaggtatttaagatcattgcgtccctgcactcgaataatggtgaagaaagcatgatgaatatatcacgataacgttcagtatgtatcgaggatagatggagttactcctctctgaaatagaattactgctctactttttctgtttccagaaattactagtaacaaaccctgatatattcttttgaaaatggagcttaaacgagtctgaaattaacttcagatattagaatgccgttggctttgtttttcaacaattcctacttcatttatctttgtcaaccattgcatttctgtagcagtgaatttcaacgttttcgtccggttggcagcaggtatttgtaattcgtatatagctgcttttttattgaaaacagtatatccatctgtagtaacagtagaagataataatacaggatttgatacttcaaggatgatattttcttgtttttgtggaaggcaagggaatatagtttgtgtaagcatattgcttccttatgttatattttagtaaagaatttattttttacattatagtcggctaaatgaagtttactgtaaaaaaacgatacttttttcgtaagcgatgagatgtggagccagaaaaatggctgcgttacgtcattgaactgtccttgacccacgatattcagacagtcgttgtatcttaagttgacattacccgctgacgtcgatattcccttggtgagagaymmmymarmrmmmwrwrmrrmwrrwwwwmmwwrwmmwkywmrmwmrmmmmwrmmmrrwwmwrmmmwrmmrwwmwyrrwwwmrrmmkrmmmrmrmwrymrwmwysmwwymwmrwwwwmmmrmmmmkwwmwrmymwymmmwwmmwmwrmkrrmwrmwrmywrmmrwyrwkmmrrrmwmymrmwwmmmmwmwmwrrmrmmmwrmmrwrwwwmmwragccgtataagccgaaccgttatcggccactggattggccgttccggtcctttatcgccatatcgctgacatgacatcctgcacygtygctttrtcrtaayctcccgtgcytgckgmmmmrwmwmwgrmttcacgatcgcaatatggaacgtaacgctgtcatcgggagaatcgtcaatgtggcattcggttmasrywrmwmwrmmwwwmmrwrwymmmmmrrmmwmwmkmmwwwwrmyrmwwwwmwwwwwmrrwwwmmwwmmmwrmrrrmrmmrwmkmmrwmmmkmyrmmrrwrrmwmmmrmwrmwmwwmrmrrrwwrkmwmsmgmmwtacagaggtccgtcatatgagcctgccactttcttacgtcgcgaatcagctttgttccgccgtcgccaagcgctaacgtgsgttactcttgtaccgggctacgtcgccttcagggaactggttattctcattttttatttgtgacattgtggttgttgtgcgacagacattgctattcggcccatatcccagataaaacccgccagctcacgtgcaacagcaacaattgtaatattgacattctttcctttggcctgaagttttcgatacctcttacaaagcctgagttgagcatcccatgctctgtcaatgacggggcggggtaaattttcctgccttttttgtattgccggactgatgcgtgccggatgacggtaactccacgctgcctcgaccaatagctttctggcatagctgtttccggctttggtaatgctgccctgatgtctgcttccacccgatgaatattctgatggcgtgataccaaaccatgacatcagttgtcgtggatgttcaaaacgggtcagatcacccagttcagaaattaatccaaccgcagtaataaactgaatccctcgcatggcttgcagcgcctctactaccggatacagcctccattcggtaacggcctccttcagggcggattccagccgttcacattgtgcctgccgatcttctattgtacgacggtgttcgtcaaaagctaattgtcgccagggactttcgaatgagtatttgctgagccagcgtcggtgtgcagggccccaatctgctcgcccgacataatggaccccatgaaccagaagaaacgatttcagacgttgccttgcatgccgtaaatcatcgcgggcagatgcccatgcccgggccaaatcccggaatgcctcatcttcaatgccgggtacgtagacagcagaaagatctcctgcacgcagtgaacgcacaagtctgatggcatcacggcgatcggttttaacccgctcccccggttttttcgggataagtgagggggcgcagaccatgcagtcaaaaccggatttcaccagccgacgataaagtccataaccacagggccccgcttcataaacgatactgacctgcgatgactttgacctgagacgtttacaaagattctgaatatcagtaggtgatgtgccaattttacccatcagttcaacaggttctgaattgatagcataagcgacagtaatcgattctttgtgaacatctaacccgacgtacaatgtgttattgttgttcatgctggcctccgtggtggaaataccggggtgtagcaacagctcacccatgcggctctgacaacgatgttaacccgcgttaaatatcccacgttggccagcatctgtctcacggaaagtcatactgccttaccaatatcaacaccaaccggatttgtggagcctccagtctgaaggggctttatgcgttccgtataggatattatgatgctggagttttgcgaagctccactcctttccgaacatttacacagatattgcctaagaaaagtgaaaccataatatggttatagtccctttcagtgcaaatacttagcttgttgcacagagaaatagaagctgcataagctctttcttcaaaaaatgtaaataaagttaaatataggaaaaattacttaatctatcttaataacggaaagattttgtatacaatcactgttacagaaatttcttagttactctgtaaacactaaatatagtttggtatattctgttgaatttatgatggatatgggacataaaaacaaaatagatataaaggttcgcttgcataactatattattttatatgcaaaaaggtgttcaatgacggttagctcaggcaatgaaactttgactatcgatgaagggcaaattgcttttatagagcgaaatatacaaataaacgtctccataaaaaaatctgatagcattaatccatttgagattataagccttgacagaaatttattattaagcattattagaataatggaaccaatttattcatttcaacactcctattctgaggagaaaagggggttaaacaaaaaaatattcctcctctctgaggaggaggtttctatcgatttgttcaaatctataaaagagatgcctttcggcaaaagaaagatctatagtttagcttgccttttatcagctgtttctgatgaggaagctttatatacttcgatatcgatagcttcttctcttagtttttctgatcagataaggaagattgttgaaaaaaacatcgagaagagatggcgtctttctgatatttcaaataacttgaatttatcagaaatagctgttagaaaacgattggagagtgaaaaattaacatttcaacaaatccttcttgatattcgcatgcatcatgcagcaaagcttttattgaatagtcaaagctatattaatgatgtatcaagacttatcggaatatcaagcccatcttattttataaggaaatttaatgaatattatggtataactccaaagaaattttacttatatcataaaaaattttaaatgcttcatagcccatcgctattgccagatgggttttccccacacctgatggccccagtaatacgatattttcattacgttcacgtataggaaattgaaaaactaagcgatacccctgtccgcgccttatgccacgtgagcctttctgtttttccccaccactccggtgttttctctctctgcatggtcgattcttataccgcaacaggctgttttttatacttgcaccgattcccgggcacacttagcctgtgtcaggccggcatatacctcagcagactgatatcccgcgcgtttttcttcagcccctccacattcagccccgcaatggcccggcggtataccatccggccgccacacagcacgcattcgaacgggtcacgactcaggaactgtttcaccatttgtgcatagcacactttcgccactggttccggtttatccatccccagagcacggtacacctgcggcagcttctctccacacacccggttggcaaggaacccgaagtacctcaccatcttaaaaaacttctccgggatgtgctgtttcagcctcgcgaccagctcacgctgtgtcagcgtttccgtcgccgtttctcccgttttgtggtccaggtaacggaagctcaggctcgcccctccgttgtaatgtgccagccgggaggccgctatcggcggcttcttcagataacgacccaggtagcgcgccgtattcctcccgccggccgtttttttcgacatgtacacatgccagtattttcctccggctttcagcaccaggcttctccactgtgattccgtcgtgatatgtgacaacgactccggcatcgccagcccctctgaccacgctttcagaagccgttgccgcatattccacatccaccgcgaacgcatcgcgtctttcaggaagctcagctttttccactgaccatgcttattcagacatccacaggttacagacacatgtacatgcggatgccagttgagacgacggccatacgtgtggatggcgcagaagataccgggttcctgcccccgtttccgggcggcatacagcagattctccaccgccagacggcacacgtcattcagcagccaccggttgctttcgaacaccggccacagcgtgtccggcagggtgaagaccagatgtacccagtcgcagtcaggaagacgattcagctgtgtcgctatccacaggtctgtggccttttttccgcaggacgggcaggcacggctgccgcatgagttagtgacgtatctgacgtgggggcactcaggtttatcgcagttgtattccttcactcccagtatccgcgtgccgcaggccagcatttttgtgacagcctcaacctcgatatcgcgcagaccgcccgcatccaggaaggacgtccagcactggttggccgtgaacagacgtttcagcgggcggggagtaaaagcggacaacatgaggacggattaacccccggtatcctgtagccgggtaagggctgctgtgtcgccacggcgctccaggatggaggccgcgaccatcgcaggtggtgggtccatgggtttgagcatcttcatccagtcccattcacgttgtgagagcttctcagcaccaacagagagcagaacggtatctatacttttgcgggtcaacataagaggatgagtccttaataaaacaggtcctcagagcatacatattctgcggagcgtgcggcaacaacaggacgaacaggggcggttttcaatttgcagccgtcaggctgccgtggttcagcgcctgatgggaatcctctcacttaaggcagcgattaaggtcaagcgataccgctcttacagaggagaggtagggcaaaccgcccccaatgttctccaaagagatttcaaggctacgcggccaaacgagaagtgggttaccgatgttactgaatttgcagtcaatgggcgcaagctgtatttgtctccagtaatagatctcttcaacaacgaagttatttcttacagcctttcggaaagaccagtgatgaacatggtcgagaatatgctcgatcaggcattcaaaaagcttaatgaaccgccccgggaatcctggagactaaacttcctgagaaagaggtaaacaggatgactaaaaatactcgtttttcccccgaagtccgtcaacgggcagtccgtatggttctggaaagtcagggcgaatatgactcacaatgggcgacaatttgttccattgctccaaagattggctgtacgccggagactctgcgtgtccgggttcgccagcatgagcgggataccgggggcggtgatggagggctcaccaccgctgaacgtcagcgtctgaaagagctggagcgtgaaaatcgtgaactgcgccgcagtaacgatatccttcgccaggcttccgcttattttgcgaaggcggagtttgaccgcctctggaaaaaatgatgccactgctggataagctgcgtgagcagtacggggtcggaccgctatgcagcgaactgcatattgccccgtcaacgtattaccactgtcagcaacagcgacatcatccggataaacgcagtgcccgtgcgcagcgcgatgactggctgaagaaagagatacagcgcgtatacgatgaaaatcacaaggtatacggtgtgcgtaaagtctggcgtcagttgttacgggaaggtatcagagtggccagatgcactgtggcacgtctcatggcggttatgggacttgccggtgttctccggggtaaaaaggtccgtacgaccatcagccggaaagccgttgccgcaggcgaccgcgtaaaccgtcagttcgtggcagaacgacctgaccagctgtgggtggctgattttacttacgtcagcacatggcagggcttcgtctatgtggcgttcatcattgatgtgtttgccggatacatcgtggggtggcgggtctcatcgtccatggaaacgacattcgtgctggatgcactggagcaggcgttatgggcccgtcgaccgtccggcacggtccatcacagtgataaaggttctcagtatgtatcgctggcctacacacagcggcttaaggaagccggattactggcatcaacaggaagtacaggcgactcgtatgacaacgcgatggcggagagcatcaatggtctttacaaagcgaaggtaatacaccgtaagagctggaaaaaccgtgcagaagtggaactggccacactcacgtgggtggactggtataacaatcgacgattgctggaaaggctgggccatactcctccggcagaagcagaaaaagcttattatgcttccatcggaaacgatgatctggcagcctgagttcacagataaaacactctccaggaaacccggggcggttcagttgctgaactgctggaaccgctgtatgacatcctgcgccagtatgtgcagatgcccggtaaagtccatgccgatgatatccccgttccggtccaggagccgggcagcggtaaaacaaggactgcccggctgtgggtctacgttcgtgatgaccgtaacgccggttcggaaattgcagcataagttatccatgctctgaggtgggtggaggtrggmmmtmmrrtcatgtcaacgccacgatgtttgaccgttatttgccattttcatgcccgctaaatactttcgttactgatgctgtccgctttccaccatttcatcatcactgattggcgttgtgtttttggtcagcactccagctttacgtttctcttttaaccggtaactctctcctttgatattcagcgtggttgagtgatgtagcagccgatccaggatcgctgttgccagtacgtgatctccgaacatttctccccagtctgcgaaccctttgtttgacgtcagtatgatgctcgctttttcatatcgacggttcagtagccggaagaacagactggcttcctctctgttcatcggcagatagcctatttcatccaggatcaacacccgggsmtmamtmrkwwrmkrymrmwrmmssymmmsmyrrywwwymwrwwwsrmwwwmmwmmrwgwmrcgatcagtctgtccagtggcataaacagtacccgatgtcccgcatccaccgctttcacgccaagagctatggccagatgagtttttccgacaccaggagggcccagcaggatcacgttttcgctgcgctccacgaacgccagaccagccagttcccggacaaccttacggtcgatgcccggctggaaggtaaagtcgaactgctccagcgttttgacccacggcagacgagcctgcttcagcctggactccataccgcgctgatgcctgccgttccattcctgctgtagcgccatgcacaggaactcccggtagttcagctcttttttagctgcctgttccagcaggctttcaacgtgataactcagatgctccattttcaggcgactcagcaggacttccagttcatgcatcacaacagctcctcataggcactcagtggtcgatgttccacctgactgacctgctgccagagcggggcgtgatgctccggcactgtctgccagccagacgatgctgaacagaggcgatgtgaggccaccagtttctcattactgtagatccgcaactcgtcatccagcgatattcgtatcgataccggctgaccacacagcgcttcgggaacgctgtaacgattaccaccaacctcgatatagctgtcccaggacacatggcggatatcgaagtaactggtatcgaagtctgtatccggtaacggctgcagatgttcctgctccagcgcgaagcgctgttccggcgtttctttgaactggcgaagctcccgtttgtcagccacatcggctatccattgctccagttgttgattgacatgagtgaagctgtcgaacctgcggtaccggacgaagaagttctccttgaggtatttcaccatccgctcaaccttaccttttgttctggccctgcgtggacggcatgcccgtggcaggaagttatagtggtcagccagcaacaggaatccggagttgaacacgactttcccgttgttattcttcagcaccgcagccttctggttatcaaccagcaccgttttcacacaaccaccgaagtagcggaaggcgcgaaccagtgattcgtaggtatgctcagcatcctgttttggtgcggcgaagacatggaagctgcgggagaaccccagcgtattaaccgcaaagttaactttgcaccgttgcccggcaacctccacctcaacttcgccccagtcatgctggagctggtatccaggctgagtttcgaagcgaactgttctttttgacggacgcattttacgtttgggctggatgtagtaacgcagcatggaacgtccgcccgtataacccattgccttaatctccgcgaagataacctcgctattccagacattctctgccaggcgcatgtcgatgtaatccataaacggtttcagcttaaccattttgtggcgggtctttctggctggcggttcagggtatttgaggtagcgtctgaccgtccgttcagagcaaccaatctgagtcgcaatatcgacgatgtacgcgccctgctggcgcatttgctttatcatgtaaaagtcctctctgctcagcatgttgatgtcctttctggtgtgagaaccttaaggaaacaacatgttgggtggagcggacaatccaaatggcgaattaccgtcttatatcactggcgctgacagtcagggtttacaggagcactgaccaaacgacccggtgctgcttcattctaaggaatgaggtggtgagcgactcgggaaaaggcagagatctactgtcacgtggggttcgggaagctgaacgaaagtaaactgtccgatgaagcatcgaaatcttagatgatgtcaaaatggggccgtacagccgccccctgataagcgcagaagacacctgcttactgactgcgcggcatccggtgtgaaggcagcaggatcccgggccagaccatctcagggaacgtgagaacctgtttccggcacgtatcgggaaacaggggcggacccattcgtagtagtgatgaaagatctgtaatgggtctggagcgaagggatggggatcacattcattgcagaagacacaacccaaaaggggatgacgtaactgatgcaacgaaaatcatttgaaataccaaaagcactggtctgggcatcatatctggatgtgcgtcgcaacaagggagcccccggatgcgacgggcagactttgaaaatgttcgaccagcaacgtgatggcaatctatacaagatctggaaccggctatgttcgggaacatggtttccgccaccagtgctggaaaaacggatcccgaagtcgaatggcaaggaacgcattctggggatcccaacagtatccgatcgaattgctcagggagcgataaaacttttcatggaagaaaagcttgacccgattttccatgctgattcatatggttaccgaccaggcaaatcagctcatgacgctctgaaacaatgtgccatccggtgctggcgttatagctggatactggaagtggacatcagtgcgtttttcgaccatgtgagacatgatctggtgctcaaagctctggaacaccacgggatgcctaaatgggtcatcctgtactgtcgacgctggatggaagcgccaatgcagagttgtgaaaatggagaattaataacccgaacacgaggcactccgcagggcggggtcattagtccgctacttgctaacttgttccatcactatgcatttgatttgtggatggaaagagaatatcgggggtaccgtttgagaggtacgctgacgatattgtagtacattgttcacgaatgagtgatgcaacaaggctgaagaacaggttatcggagcgcttctcggaagtcgggctggtcctgaatgcagggaaaacgaatatcgcctacattgacacgtttaaaaggagaaacgtcgcaacgagtttcacctttctcggatatgacttcaaagtgcgtacgctgaagaatttcaaaggcgaacggtaccgaaaatgcatgccgggtgcgtcaaatgcagcaatgcgcaaaataacagaaacaatcaagaagtggcgtatacatcgctcaacagctgagagtttgctggattttgcgaggcgctacaatgcgatagtgagaggctggatcgagtactacggaaagttctggtccagaaatttcaactatcgactgtggagtgcaatgcagtcacgtctgctcaagtggatgcagtctaaatacagactttcgaaccggaaggctcagcgaaagctgacgctggtaaggaaggagtatccgaagctatttgttcactggtatttactacgtgcatcaaatgagtgatcaagagccgtatgacgcgagagtgtcacgtacggttccgtgagcagccggggagtgaaattctctccggctgactcgactcgcgggttccgacagcgatgacgaacatgcggcggtgttgtactcgctgatcggcacatgccgtctgaacaatgtggaaccagaaaaatggctgcgttacgtcattgagcatatccaggactggctggcaaaccgggtacgtgatctgttgccctggaaagttgatctgagctctcagtaaatatcaatacggttctgacgagccgcttacttttggcgtaaatctgctgcgcctttactgaacacaatgccgtattcagcaagaagtccacgaatgtgatttatcagttgtgtcctttccattgaaaccaaccgacgcatattcaaaaaagtctggacgtcctgttgctccagagtttttattggtacgggtcgtatagttccatgttggcaggcctcagctattgcctgtgcgtcgttataatcatttttctggcaccgctgataagctttaacatgttgtgccggaagcaatattacatcatggtcgagtttcctgatttcccgggcccagtaatgagatgccccgccagcctccatcgcaacagtcattttaggctgttgcaccagccagttaagcagttgattgcgagaaagtttggtctttcctgcgggtttgccatgagcattgatactgaaaagataaaagttcgttttagccagatcaatgccggtgattttctgttcagacatggtattccgcttacttttgtggttaaagtttatccatcatggcgcaattgaaggctgccgtgaaagagggggaagactatctcatcggttactgttaaacggctaagagaaactatctttttcaagcaaagaagatacggtgaaacaggcgatttcaccgtgaaatactgttcaaaaaaaacgctaataaaagcaaaatttcttttaaaacagccaattaaaaactggtgagttgctggcgaaaaacttgaattttcacagcgaattcagattatcttttagtacaagagcaatcatacgatctaacttatcctgaacttccaacggtaatctgccgaattcatatgagaacgtacgtccctttactcgttttcgagcaaatttatctttactatcaaatttccataacaaagttacaaccgctttatcctttactcctgaatcaagcaatgaagacatttccttggttataagccgcataatactgtttttaacctcgtcttcagaaagattgtcatcagcagtaataagctcaatactcggcgagatattcttcacaacttcatcaatagatatatcacccagcctcagtcgttcacttaattcagccagttgcctgtaatcggcgtatgtcagctcagactgaacaggaaaaagagacacaaaatctttaggtacggacgctgcttggagggcacgagtcaccttagctgccgacaatccctctctttccgcaatttgcttctggttcataccggcctctttgagacgtactaaacgcaacccaatttcacgaatattatgttcaagtgacgtctgcaaatctttcgccagatgctgagcttcagaaacagtaagctcatcatccgttacaagtacacgtaatccaaccttgcatagcaaagctgctgcccgacgacgggaaccatccaaaatctcaataagatcgccagtccgaatcccaatacatggatagaattgttgtaaatgtatagttctggtgatgtcttttagtgagtccggcgtcagggctgtctgttcgcggccattcacctcttcaaccacaaaagtttgagtgtccactttgtcatgatcaaccttgatctcagtaaattttgcctttcggccagtattgagggtaaatacatgaactggagccgatatttcttcagtattattcaggatatttgtattcagcgtacggccaatagtcggtcggtgctttctgttttccatcagtagttggcccttataaattcaatacgatcaaaaactgctttagcgaaatcttctgctgccatacgtgcatttttcaaagcttcaccactgccaacataagtaacaggattcgcagatatcacggtatcaaacgactcgccgcttctctcaaaaccatcaagacgtggcattgaaacatcaagcatgtcaccaccgaaaatctcttttgtcaggctatggcagtatttatgatctgacttattcgccagtttcgacatgaagccaatatttgcctgcaaacggcatgaacatccatcctgttcaataatctgtacaagttcaggtaaacgagccaaatatttaagcgtggaatggaaatcaacttgtgctggcggcaccggtgtaaacatgatatctgcggcagcaatggcatttttcaaaaatgcgtccagatgcgggcctgtatcaataaggatgaagtcgaaatcatgcttcagcttatcaataatattctcacgcaatatcgcgtgtttgttttgtcctaatagatgctcctcgcacagagtatcccagtttgatgcaataaaggcgtcgtcaatagaagccggcataacataaacgccagggataactgacggaacgataaaatcttccagtagttcctcacgactcacgttctgcaacatagcctgcggtgcggttgtatcaaccagccccaccgcatgcaagtagttcagaaacatagtggctgatgactggggatctaaatcaaggagcagaatacgtaaatcttctgacagcaggtgaggatgagcacgcagggcgtgagctacggaaacactactcacggttttagacacaccgccttttagactaccgatgaatatggaatatgcctcactataacgatcgcgatatttaggaatgccacgatgtgcatagatatcgataatgttctggatggttaatgcatattttgtcgcggttcctgctgggcgtttttcaaactgatagccctgagcctccatctcaccgactgcaccatccacgcttgcgcgcgttaattttggaagcttagctacagcagactttgaatagacttgataaaaaacatcagcctgaaactcctgcttttgctcctgaatctgttttgttagggcgagcaacattttatctgcacgctgcgcaaccttacttaactgctcaaaactagtcattccccctccgtgctatgtaggattttaacttattacaaaaaaaactgcattagcagggtaatgcatgcaagtacatattgtacatgcttatattaaaaatgcgcgcattgctgatttattaatcatcggaatgaatattactgtaagatagtatgcctttccgggagaaagacgctgatttcccccgaataatccagttcaccaccacactccggacaacagctttcttcaggctccagatggtgtatttcgcgggaaacatgttccggtaacggacggcgatgacgtgactgtcgcaacaggcgggggtagtgcttccaatcagtagaacatgtgcttttcaatgaaggagccgatgactttttcgtggatttccacggaacgcgagaagcagattgttttacgtgccagccgcttgatgcgggtgcggagggtccggttgttacgttcaatgcgttgcgtgaagattttgccggtcaggtgtttctccttcggcacttctctggcgtagctgccccagtcatcgctggtcaccatgccaccatgccgatacagaacggagtcaacagggctaacagttcccgacaggtctcatcattccgtggcccgaaagtcgaaagtgtaagccaacacaccgccagttttagtgttgtacgcataccagagccagtgttgtgtcaacgacggatgaaaagtgatccacttatatctccaccaacggcccaatattgatccaccgttttactcaggattagcttctgctataaccccggcctttcgtttctgtctgagtcgatagctttctcctttgatttgaacgaaataatccgacggtgaatcacgctgatattcgcgggttggataccgcattacagaggtccgtcatatgagcctgccactttcttacgtcgcgaatcagctttgttccgccgtcgccaagcgctaacgtgggttactcttgtaccgggctacgtcgccttcagggaactggttattctcattttttatttgtgacattgtggttgttgtgcgacagacattgctattcggcccatatcccagataaaacccgccagctcacgtgcaacagcaacaattgtaatattgacattctttcctttggcctgaagttttcgatacctcttacaaagcctgagttgagcatcccatgctctgtcaatgacggggcggggtaaattttcctgccttttttgtattgccggactgatgcgtgccggatgacggtaactccacgctgcctcgaccaatagctttctggcatagctgtttccggctttggtaatgctgccctgatgtctgcttccacccgatgaatattctgatggcgtgataccaaaccatgacatcagttgtcgtggatgttcaaaacgggtcagatcacccagttcagaaattaatccaaccgcagtaataaactgaatccctcgcatggcttgcagcgcctctactaccggatacagcctccattcggtaacggcctccttcagggcggattccagccgttcacattgtgcctgccgatcttctattgtacgacggtgttcgtcaaaagctaattgtcgccagggactttcgaatgagtatttgctgagccagcgtcggtgtgcagggccccaatctgctcgcccgacataatggaccccatgaaccagaagaaacgatttcagacgttgccttgcatgccgtaaatcatcgcgggcagatgcccatgcccgggccaaatcccggaatgcctcatcttcaatgccgggtacgtagacagcagaaagatctcctgcacgcagtgaacgcacaagtctgatggcatcacggcgatcggttttaacccgctcccccggttttttcgggataagtgagggggcgcagaccatgcagtcaaaaccggatttcaccagccgacgataaagtccataaccacagggccccgcttcataaacgatactgacctgcgatgactttgacctgagacgtttacaaagattctgaatatcagtaggtgatgtgccaattttacccatcagttcaacaggttctgaattgatagcataagcgacagtaatcgattctttgtgaacatctaacccgacgtacaatgtgttattgttgttcatgctggcctccgtggtggaaataccggggtgtagcaacagctcacccatgcggctctgacaacgatgttaacccgcgttaaatatcccacgttggccagcatctgtctcacggaaagtcatactgccttataaggctttccagttgcaactgcccggcgagcgccatcagtcgttgatgttgcagttccatcatcacgccactcctctgcagaatgagtcgtagatggagagtggatgatgctggggggtgcccctattaagttgtcaagcatgttatgacccctgcggggtataaaaagtcccgtcgcgcatcatggcgaacagaacgtcgcagcgtcgtctcgccagggcgataagcgcctgattgtgtcgttttccctgactcattttgcgggtgtagtaagccctggagagcggatccctgagcgcggcgaaggccgacaggaacaacgcccgtttgagagctttattaccccgtcgcgagggatgctcaccgcgtattgacgagccggatcgccgagttaccggcgcaaggccagcataagcagcgagatgtgcggcagaggcgaaggcgcggcaggcgacctcggtgaggagtctggctgcggtcctgacaccgactccgggcatactggtcaggaccgggtaaagagggtgagcaagaactcgctgttctacctcaagcgccacctcgtctctttgcttacgcagcgtgatgagctggagtgccagacgtggcagtactacggcagcggcattcgtgccgggaacgacgacggtttgttcggccagtgcctgagctatgtctgctgcaaggcgtttacccagacgaggcgcaagtttgcagagctgggctgccagcttcttctcacccagcgaagcgagtttttctggtgagggatatcgctggagaagatcgagtaccgccgggtgctcaagtctcggaccgagaacgcgctccagtgccggatgtatctgggtcagaaggccgcggatacggttgctggcctgcgttgtctgtgcggcaagatcatcatcgaagccgcagagcatggagagttcggcgatttgctcgtcagccagtttcagcgtgcgtagcgcgtgaggcagggtacgggcagcttcggcaatgatggcagcgtcacgagcatcagttttagcttcaccggcgtgtaagtcggctatgcggcgcatggccagtccagggaggtatccgacaaggactccttctgagcgggcaacggcgacaggtaacgcaccgatggtagctggctgatcaacaaccagcagtatctgaccatgttgtttcaggtcagatattagcgacctgagtttgttttcgtcgttgggtaatgctttatcgaacaggcgtttacctgaacgattaatggcaacagcgtgatgcgtatctttaccgacatcaacgccgataaagacctggacggattcgtaatcgctggattcggtcattctgtctcccttgtatatgggttaaccagataaccacggggagcaggtaccggcatccacgttacagacggtcccggcaaaagtgcctgacctgacccctattagcggttaccagcgccccaccagacccggtgacatcaccccccggatcatggacgactgggggcagtaatcatgccgggtctggctggctaacaccccattataaggggtacgaataaagtaacggggttgtcgaagctcaccagattttcaccaggatgcacgtcatactcttttttctccggaggcagtgccagcatggactgctgctcttcgagccagcgatcacagggacgggcctggattgtttcatgctttcgttggttagcgacatcgtgcagccagcgcagaccgtggcggttggctgtttcaacatcgacagtgctccccatcgggcgcaggcgagtcattagtgggatgtaaaaactgttacgggtgtactgcaccatccgttccaccttacctttagtctgtgccctgaaggggcgacacagtcggggagagaagcccatctccttgccgaactgccacagcgaaggatggaaccggtgctgaccggtctgatatgcgtcacgttgcagaaccacagttttcatattgtcatacaacacttcgcgcggcacaccaccaaagaagcggaacgcattacgatggcaggtctccagcgtgtcataacgcatattgtcagtgaattcgatgtacaacattcggctgtatccgggaacagcaacgaacacgtgaagcggtgagcgaccattacgcatagtgccccagtcaacctgcatctgtcgtccgggttcagtttcgaaccgaacggcaggctcctgctcctgaggaaccgagagagaacgaatgaatgccctgagaatggtcattccgccacgatatccctggtctctgatctcgcgagcgattaccgttgccgggattttgtaaggatgagcatcggcgatgcgttgacgaatataatcccggtattcatccaggagtgaagcaacagcaggtcgcggcgtatattttggcggctcagattttgcctgcaaataacgtttaacggtattgcgggagagccccagttctctggcaatcgcccggctactcattccctgcttgtgcaggattttaatttccataactgtctcaaaagtgaccataagctctcctgaatcaggagagcagattaccccctggatctgatttcaggcgttgggtgtggatcactattgcaccgttcgtgacagtgttgttgcgccttgctgccaacgaagctccactgcccatcaagttcacagataagggcaacatcagcgtgagcgactggcgcagatgttatccgccggggcgggagctttttaatgtccgtatgackgtattratgccgattttcagcgtacgggcagtatcgcgaacyccggcaccgttgaaagycawtwmmryratttggtytttaactcccggtttacgggsctcgtaacagtmastgagctgaaayacccggtgacagtcacggcmrcggaatcgmtcatgtcyttttgggttctgcccgtgacggtaaacctgaacggactggcaacgggggcaatgaarrtwmmctrtgaccatgaaasaaccttaaaagcgrgtattatawccyctaattcaactaattggaggcattgtcatttttaatctatggatacacttaaaaatcctctcaccttacgttaaaaaccttctcatgtttagagttttttattatgtttatcataatgatatgacgatctgagtttttaaaatcaagaagaaaagataagttttttaaatgcacagtgcctgtggataactttgcgtaaatccggaggacagatcactctgaagcaagacaacagcatctgaaaacaatatgctcacgtttcaaccacagggcgtaagtgatcctacccacgtaatatggacacaggcctaagcgaggttctggttttcaaattgttccggactgaggccgccacaccaactgtgccgtcgccagcgattgtaatcacattcgatataattaaacactgttgcccgcattatttcccggctgataaagtgttctccgtggagacattccactttcagcgaatgaaagaagctttccacgcaggcattatcgtagcagcagccctttgcgctcatactgccacgcagattatgccacttcagcagcgcctgataatctgctgaacagtactgacttcctcggtccgagtgaacgataacgttccggggtctcttacgccgccacagcgccatctgcagggcatcgcaggccagttgcgccgtcaggcgtggtgacatggaccagccaataacggcacgtgaccacaggtcaatgaccacagccagatacagccagccttcatctgtacgtaagtacgtgatgtctcccgcccacttctggttcggaccactggcgtaaaaatcctgctccagcagattttctgacacaggcaggccatgtgcgcggtaactgaccgggctgaacttccgggaggcctttgccctcagcccctgacggcgcaggcttgccgccacggtttttacgttaaagtggtaaccctgagcacgcagttcatccgtcaggcgtggtgcaccgtaacgctgttttgaccgggtaaaggccgccgcgaggacaacgctgtcgcagtgctgacggaactgctgacgagggcttatccctgtccgccgctgacaccacgtataccagccgctgcgggccacccggagcacgcggcacattgccttgatgctgaactcagcctgatgtttttcaataaagacatacttcatttcaggcgcttcgcaaagtatgtcgcggccttttggaggatagccagttcctctgcctgctccgccagttgccgcttcaggcgggcattttcagcggccagttcactttcacgctcagacgaggtcatctgttgctgcagtttgctgcgccacgcatagagctgagattcatacaggctgagttcacgggctgcggcagcaacaccgatgcgttcagcaagcttcagggcttcactgcgaaattcaggcgaatgctgtttacgtgtttttttgctggttgatactgtttttgtcatgtgagtcacctctgactgagagtttactcacttagccgcgtgtccactattgctgggtaagatcaatttgctcactggtatttactacgtgcatcgaatgagtgatcaagagccgtatgacgcgagagtgtcacgtacggttccgtgagcagctggagagtgaaactctctccggctgactcgactcgcaggttccgacagcggtgacgaacatgcagcggtgttgttctcgctgatcggcacacgccgtctgaacaatgtggagccagaaaaatggctgcgttacgttattgaacatatccaggactgaccggcaaaccgggtacgcgatctgttgccttggaaagttgatctgacttctcagtaaatatcaatatgattctgacgagccgcttacctttgagcagagaaaggggagagggatcccacatccatgagctaggaaaatacaaaaactggctatacaacaattcaatatattaggacttacttgcgaggagaaatatagcaccgctcaggcaattcctgggtatttacttttcttagtttctttactcgggatgttctgtcatcagaaaggcgattctaaatttgtaatatagtcactcatattcccacgtaatttctttagatcaatatattgcacatttttctcaaaaaactgatcggaaataagatttgcaacattgcttgttgtatcatttttaacgcttctattcatcccttcacaaagttcgtccattttctttttgaaataactgcttcctgatacaagatgatttatggaactttgtgcagcattgatgtctagagaaatgcgataatctttctcaacctgtttaaatatctgtttacggaggttatcgccgatttctcggctgtgttctgcacaaaacttatcaatacattggtttaccacccgggatacgtagacttttttttcatcatttttccaatctgtaatattttcaccgatttgttctgaaacagagctcaaaatactagtatttattttggttgatgatatttttactatccttttccccattgtacagcggataatctcatccacctttcctgaaaaatatcttttgtgagataaactgattccaaaattattaaaagatgttccaagcattataatcactttgtcatgtaaaaatagtaacattgtcattattttagctgattcaaacaattctgtaaatgctttgagttctttactcccttggctcaatgatggaccgtctggaatcccagagactaaactctctgagaaagagagtatgaaaaaacttggggcagtccctaaatgaagggaccaacagcggtaaaataaccggcatcattcataatgatgccctgttagttgtagttatgaagatttacgtcattaaaattgctgttcgtagtgtcagtcctatggtctggcatcagctgaggatcgtagccgatacatcactggcttcacgacattcgtgttgaggctattcatgaaaactccacgctgaaaacgccgttttgtataaacggtcacggtatgcccggagccactgttgctgatgaattcgataaaaccttggcgtttcttgaagctatcgttaatgcggataatgaaacaacgattggtgagattcgctcttttgccgacgccctggacgctgtccggttcaatcgcaacaaaatcaaccggcagctgagcaaacccaaccttgcctctctggcgctggagcatgaagtgatttggctgggacgtagccgctgacagcttctataaaagtatgatcctacccacgtaatatgaacacaggcttaagtgaggttctggttttcaaattgttcaggactgagacctccacaccaactgtgccgtcgtcagcgattgtaatcgcacttgatataattaaacacwrymwmwwwmmwwwrwrrmrwmwmmmrwmwrmrrrrrmwwwmwrmmwymmrwmwwrrrmmmwmwrmwrmmrrmmmwmwryrmrrmwmmmmammytcctgtatttcccttcctctatcacagtgcactttacttaatagggtgggatgtgttcattacatttattgtgttttttatgattgtttttgcgtccagtgtcgttcgtcatgggaaactggttctatgggtagtatcgctcagtctagttaactgtttgtttctttttagaaattaagcagatcacacaattcggggagagatccttctaagtgcaattctttgcagctatttagcacacttaaaacaactcttgtattttgtcagaaataatattaaacatgtcttgttcattattgttaagtctgtctgctatttctgtaagttttatcccactatgatggcatatatttatatatgccatcatagcctcgatattccctttccctgcggccagaaataatcctggtaaaccctcattatctcttacatccaatagtttaatcgtttcttttggtgataaatcaaaagttttaataaaatctccataagcttgaatcgcagcggaatgaccttcttgaagagctatgaacaaagcagattccccagcattatttcgtgctgcaagaacttctagtttttcctctttgggtatttcggctgccaacagcgattcaccgtatttacgcgctgcatcggcatttccatgccaaatcaatgaaaacaaattatggcggttatactccttattattctttgtcggaatggatatgctctgtattttggccaattttctgaaagaaacaccactatatcccccccctgatttttcttcattgatggcgtgggatatagcggaatttgtcacgttaatgatattacttttcattcctttattttcattagcagtattgctgcctattattaggtgcatatttttgttgtttgctggatgtaatccatagttatttattgacatttttgttcatcctttttgctaaaaaaaacaataacctatttttgtaatgtaatagatattagtctattgccaatctaaaaaaaccattgccacattgaatggctatattcaccaataaattctttcgatttcgtatattgacatatcgtagatatatcttatccctaagattcaataatcgagtccgtttaccattctgtgcaagcgccttcactcagaagtgaccgtcaaggcgatcaccaaacttgataataaagcagctcattgccatacgccagtccttaaggggcgttgtccatttctgtgatgctgcctagatttaactgccagctccggcttatctacatctaaatatgtgagcctaataccgctacactttggccagactatgtttttctccgggaagtgggctaaccgtctccataaagtgacgaacttttttcaacagtggccatattgaacggcactgatgattacgggttattgtctcatgaagagccggccacatcactgacagcagtgacttcaggccttcagcatcagacagcctgaaccagttaatccatcgcccgacagatttagtaatctagggctggtttttcttcagctgttgcggttcgaaagtgccattacgatcacgcagagtacggatttccagaggttcatctccagtagtgataatcttcgttaagcaaccattgcgggaattgctgccagatctaagctgatttttctcgcacccaagatgatgagtcatattagcattgagtacggtttcattttcagcatccaatcaaactgactgagatcttcaggatttttgagacttttggctagttcattagctaaagcccgcaacttattagatgtcaactgtttttcattcataaattcaccttcatttgatatcaaattaaatagttcaacatcagagaattatacaaacttatgtacaggctcctatgatcttcgaatcctgaagattatagtgaaaataagttttcacgttgaaatcccacatagttagtatatgataaataatcatggatgacctgattaacctcttggggtagtgcttttatagaagaataaatattctactaatttaatacatcacctaaaatagtattgttttttatggttggttgaaggtcgtggtgatgatgttagtccttttgagaatattccagtgccagatcttacttataataaaggggcttggcagtttggataaactattgttcttattccactaatacaaactttttgtaaagtatattaaatatacttcatttcatactgaaatccccggaagtattcaatcaaacaattacaccataaaaggaagagggggccctaattaagttgtcaagcatgtttatgatacctgcagggtataaattttctggctgcaagcagacacatataaaacgtctttattgatcctacccacataatatggacacagtcgtaagtgaggctctggttttcaaattgttccggactgagaccrccacaccaackgtrccrtcgccagcgattgtaatcgcactcgatataactaaacactgttgcccgcactatttcccgatggataatagtgttctccgtggagacattccattttcagcgaatgaattttcatctgttgctgtatccagtcatacagtgactgcatcagtggcgcggctctggcttttcttgccgccagacgctgttctgctgaacatccccggacttctgcctcgatggcatacagttcaccgatacgctgctgggcttccgttattctgccggattcgtacaacgcccggtaactaccgtaagcatcggcctgaagcactccgctgtaaccggccagatgattctgtggatggatacctttccggtccggtgagtacgcgaaccagaccgccggaggcatttccgaaccggcgttacggtcatcacgaacgtagacccacagccgggcagtccttgttttaccgctgcccggctcctggaccggaacggggatatcatcggcatggactttaccgggcatcagcacatactggcgcaggatgtcatacagcggttccagcagttcagcaacagcacctgtccagcgccccagtgtggcacggctcagctccacgccctgacgacggtatatttctgactggcggtataacggcagatagtctgcatattttccagcgacaacatgggccagaagccccgctccggcataactgcgtgcaatgggttttgaaggcaccggtgcctgcacgatatggtcacaccggcaacaggccagtttcggacgttgtgtttcgataaccttaaaggcgctgctgataagctccagctgctctaacacatcacatcccagagaactgagttcgccaccacaggccgggcagcattcctcttccggacggatgacccgggtttcacggggaagtgaggcaggtaacggtttatgggctgaagactggcgcaggggagatggcagtaccgggtcatattgctcacccagcgtttccgccatttcttcctgaagtgcgctgattcgctcctgtgcttcctgtatctgccgttcggtttttgcgcgaagtttttctgagcttttaccgaactgcatgcgctgcagtttcgcaaccagcgccttcagccggttgatttcggaagcataagccgccacccgctgtgagagcaggcggttgtatttcgccatctggcggatggtgtcctgttgcgtctgcaacagtgcccgcaggcgggcattctcatgagcaagtgaggtgtccatatcctcactttacaacgggttatatgcggatgccagcgcgttccgttcgtttcgggtgtttccagtcgatgccctcaagaagcatggataactgggccggagtaaggtgcaccttgccgtcacgggtgaccggccagacgaagcggccccgctccaggcgtctggtaaagaggcacagtccgtcactgtcagcccacaacacttttatctggtcaccccggcgtccgcggaagatgaacagatgcccggagaacgggtcatccttcaggacgttctgaacttttgaggccaggccgttaaagccatttcgcatatcggtgatacctgcaaccagccagatacgcgaacctgcagggaaagatatcatcagtggctgctcccttttatttcgcggataagtatctgtaataacgccggagtcagtttacctttaagcctgaggaaagggagcgcaatatgaagagctcagaaaactgggtaaaggcgatcatctggtgaagctgaaaaccagcccgcaggcacgaaaaaagtggccgggactgggaaatgaagtgacagcccgcctgctgaccgtgacgcgcaaaggaaaagtctgccatctgctgacgtcgatgacggacgccatgcgcttccccggaggagaaatggcggatctgtacagtcatcgctgggaaatcgaactgggatacagggagataaaacagacgatgcaactgagcaggctgacgctgagaagtaaaaagccggagcttgtggagcaagagctgtggggtgtcttactggcttataatctggtgagatatcagatgattaaaatggcggaacatctgaaaggttactggccgaatcaactgagtttctcagaatcatgcggaatggtgatgagaatgctgatgacattgcagggcgcttcaccgggacgtataccggagctgatgcgcgatcttgcaagtatgggacaacttgtgaaattaccgacgagaagggaaagggccttcccgagagtggtaaaggagaggccctggaaataccccacagccccgaaaaagagccagtcagttgcttaactgactggcattaaggccgccggggacgtttttttaccaccgggagtttgggcataacggtgctttcagtaagtacgacaggtactgtggaggcacttccgtaaagaagggcatcaagttcctgctccgacatacctgcgggcagaggccatgaaaggccagcgttgcggaagcgcacgaacataccacagacagttgattttggtacgcccaggcgacgtccggccacaacccgtggtaaatgttcttcgaaatgaaggcgtaaagcttcagtgatccaggtccggtattccatacgatagtgtccacttcaaaatgatggacattatttttgtggagccggaggaaacagaccagacggtttaaatgagccggttacgaagaagctgaggttctgcgctttgataattttgacgatgccgttgagtgttgcgcagacctgaatattccgttctttgtggatgccggaaacaaaaaactggtcttctggtttgtacgtgttgatgacgaagggtatccggaaattgcccgctgcatggagcgggaatttgcgacaattcctgccggtatcagcgccgacggcatgtattgccctgaatgcggtacggttcactggccggatggcgttattccgcctttctgatgcttccccgttttgccgatatttttcagcagggaaaccgatggcttaactggctggagaagcagtcggtgcagatgagccgcctggaacattatgccggtcaggatgaaattggcctgcgttacaacagccaccggacaaaacgagaagaaaaccttgtgatgagtggcgacgagtttatggaaaggttctcctggcatgtggcggataaggggttccggatggtgatacggggtcctgagtccggtgaagcggcgattactggaagatgtggtgtacgccataacggagacagtgagaaaaacggcgaggcaaatcacaaggagagggatgtatcagcagtgactgaaggttgacccgctgaagtgcgagctgtgcggaggtcagatgcggtttacggggctgaagaggggttaccgtctggcagagcaggttatgatgcatgagccgctggctcgaatacggtggtgcggctgagagtcgcagaggggaagttgcgtccatgttgaggggaacggagcaaaaaaccatcagagatacattgaatcaatcaatgtaagcaatattaagtgtctataccgattacttgatgatcaggttgtgttgaataaacgcgcattcttcggtgcttttttaatcttttggggatttaaattcctatcgatgaactgtcgatatgaaaaagcgagcatcatactgacgtcaaacaaaggggtcgcagactggggagaaatgttcggagatcacgtgctggcaacagcgatcctaaatagctgcgcttaataccgctacacttttgccagcccatgtttgcctccggggaatgggctgacggtttccataaaatggcgaacttttttcaacagttgccacattgagctgcactgatgattacgcgttattgtgtcgtgaagtgcctgccatagccgttcaacatgattcacccatggcgagtaaaccggctgataaatgaccctgaacttcgggttctccttcagccagctctgtgtttcccggcttttgtggataatgtagttgtccacgatcagcgtgatggttttcgcccgacggtatgtcgctttaagccgcttcagcaggctgatgaacagcgccgaacttttgctgttgccgcccacacagctgactttacctgtcccgctgtgcagcgctccggccagataatatttttcattctgtcccggcgtgaccacccgtttttgctgtccgcgcagttgccagtccgcaccgattttgggattaagatggatatccacttcatcttcataaaagaccggatgctctgcgctgcattcgtccagtgctttatggattgctgccatcttttcatctttatgcgggtcacggatacgcagagttggcgcagcccttcgccacacaatccccgcagacggcaaccagcggcgaacggttccggcatttaactggcaaccggttatctcattgatttttattgccagcagttctgtactccagcgtgaacgctggtagccaaagtcgccgggagaatgttttaccagctcacgtaacagtgtgcagatatgctcaaacggccagcgacgggcacgcccggcaggtaatgatttcagtccctcaacacccgactgcgtgaaccagttaatccagcgtccaacagaggaacgggcgcagcagagcgttctggcaacgtcgctgacacggtcgccccggtgcagcatcagcatggcagtcagtctgcgggcataatttttatcgtgtgttttatggatggctttctgcatcaggcgtcgttcgtcacgggaaattggtgctatgatcggcattgctcagtccggttggtgatttgttttgatttggcgattgatcagatcgcacaatccgggctaagttccctcaaagtgatctactattccgcgcagctatttagatcgrmwrcwmmmmrmtgamtatmmmmrrarararwwacmgrtwmmaagagaaacgtaaagctggagtgctgaccaaaaacacaacgccaatcagtgatgatgaaatggtggaaaacggacarcatcagtmacrmmmrtmtcttagcaggcatrmraatcgtccgcatcccctaaacggtcammatgcttctgccgtgaaggagggagaatgccatctcatcacttatatctggcgaatgaaacacagatggtcggccttgcccccaaacacacggcggtgcaaaatggaatggcagaaaacttcgtcaaaacgatgaaatatgattaaatcagtgttatgttcgaacctgacgtgttaacagcagtaaagtagtctcgcgaaggtgttcaaacattataatgaatggcatcctcatagtgcgcagggttatcgcttgctgcggaaatatctgaagcgacgtaccagtaatggggaaaatgatagctggtttctgaaaatatagaggaaaatgtgatatatatatcctgttaatatttaaggagaatataaaataaaatgaaagtctcgtttaagtctttaggctatatttttcatgatatatataataaaaaacacactatcgatgagttcaacgatgtggtaagaaaagcagttttatcaggtaaaattaacgagctgaatgcatgtcataaagtggcaatttttcttgccgaaaaagacaatgagataacaaagaaagacaaagccaagataatagatacattaactgaaaactattccattgaatttcaacaattaatgaatattagtgagcgcacgttaaattcatctttatatattactcctggtgaaagtggttttgtcagttttgttaatcgtgaaggaaaaatatgtcatactgcatatgtaaaatcatcagataatagcatggcatattaccatgcaaattatagcagcattgacaaatatattacggatatgtgcggccttatttgtatgaggcatattgagtctactggcattattttttatatgcttgatgaaaaagttttatctgccattgctgaatttatgaatgaaaaaggatggcgggctgcgttttgttcagcaaagaacttatataaatgtgtttaattttagaaaaactatttggtaaaatccaacaagagtcgagaggttcgcgttgcttcagcagggcctcagcctgttcaaccagactcagttgcaggtcgtgacgcagatttagtggtggtggttgattgtcagataccagccgatgatcttgtagtgcatatcatcagactttgagaagcatatggttttgcgggtaagccgcttaagatgtgtccgcaggttcagattatgtcgctctatccgctgagtgcatttcttgcttataacatgactggttgaggacaataaagttcggtataccggctcaggcatcggtcatgtagaaggcaatgctaaactggctcagcaaactaagagcagcgcgcatccacttacccaatgcggttgagaaaatagccttcgatcgctttcatgtggcgaaacaaccgggtgaggtggtggataaaacccgccagaatgaacatccgcatcttcctgttgaaagtcgtcgtcaggcaaagggaactcgcttcctgtggcagcacagcaataaatggatgaccgaatccaggcaggaaaagctgatatggctgcgtgcgcagatgaagctgacgagcctgtgctgggcgctgaaagaactggcaaaggatatctggagcagaccatggagcgaggaaagacgcaacgactggcagagatggctgtcgctggcggccaacagtgacgtccccatgatgaagaatgtggctaaaacgatagggaaaaggttgtacggtatcctgaacgccatgcgccatggcgtatcaaacggaaatgcggaagcactgaacagcaagatcaggctgctgagaataaaagccaagggctaccgcaaccgggagcgctttaagctgggagtgatgttccactatgggaaactgaatatggcgttctaagccttcccaccatgatcggagaagacccttacgcttacatacattacatataatgtatcgttttataaaagatgatagacaatcaatataagataatatatctattttatagaggacgttttctatgcccataaaaaagccctgtctaaaactaaatttagattctttgaatgttgtgaggtctgaaataccgcagatgctttctgcgaatgaaagattgaaaaataacttcaatatattgtacaaccaaatacggcaatacccagcgtactatttcaaagtagcatcgaacgtgccaaattactctgatatatgtcaatttttttctgtcatgtaccaaggattccagattgtgaaccatagtggagatgtgtttattcatgcatgtcgtgaaaatcctcagagtaagggggattttgttggggacaagtttcatattagtatagctagggagcaagttcctctggcattccaaattttgtctggtttattattttcagaggacagtcctatagataaatggaagataactgatatgaatcgagtttctcagcaatctcgtgtggggataggagctcagtttacgctatatgtaaaatcagatcaggaatgctcgcaatatagtgctttattacttcataaaatacgacaatttataatgtgtctcgagtctaatctattaagaagcaaaatcgctcccggggagtatccggcgtcagatgttagacctgaagactggaaatatgtcagctatcgtaatgaactacgaagcgatcgagatggaagtgaaaggcaagagcaaatgttacgagaggaaccattttatcgtttgatgatagagtagagaagatgccagctacaccaccttgtagcttgaaaactggttagaataactacgattagatttggcactgcgtatgcagcctgattcctgcccgtgacagagcattcggctcataaactgctgagctactcatctgaaatccgaacagttgtccaccacttcagcgtgcatctcctgtttgagtcgtttgattgcttttcgggatattaacctgtcggattttgcattagttaatgctcccatcaggccttcatcttttccagtgcctattttcctgttctccttttgcacaatataatcagatcatctgtatcgttgacgagctcgctatcttataggagcattctgaataagctacctccttgtaaggaggcttttactattagtaactgcaatctaatagtattataacatctctatggttgtcttctatgccgttaaataaaacattttcatctagtatattttcaacaaaaaattcgttatcaacagatatgtctgttaacagagataatagaacaataacaagttctattatgagggtctcaaactccagtgaactaattcagtttaagaataaaacagcaccttatttttcagaaaagagaaatgtgaaggtaaatataaatggagtggcaaaagatatatatggaagacaaatagtttgccgtcacctagcttcctattgggaaatgaattttatggaaacaaatgggaaggtaaactatcagcttcttagcacccctgatgctattgcaaaaaatgtatgtttggaaaaaacggaagatttttcaaaatcaccagcttatatttattttgtggaaaataaaaagtggggaacagttattactaattttttttataacatgaaaaaaaatggagactttgtcagaacattatcagcatgcacattaaaccaccaaatggcgttagggttgaaaataaagcgcgtacaagaaagtgaaaaatgggttgttcagtttttcgatcctaatcgaactgttactcataaacgtactgtatttacttgtgatagccatttcgaactgagtcaattatccgctaaggatttttttgatgatttttattggaaaatttatggtctggagcaacccgggcaggtaatatttgaagataggcataattcccctctaacaaacacagtgaaacttttaccagatgaattgatcaattccagagttatatatcatgcgataacaaagaatctgacagaagtattatttatactgatggaaaaatacaaaaatggtgaaatttctcaatctaaactggtgaaccttctggccacgagaagctctgatggtactcctgcattttatatagcacttcaaaacggatgttcagacattattcaagtatatggtaagatattaaatatgtgcaatcttagtcaagaaacaatacttacattgcttgctgcggtaggggcaaacaatgtgccgggtttatgcatgtcttttatgaacggacatgtagacacaataaaagcctatggggaaatagtctttaaaaccccgttaacttccgataagagattatatctattagctgcgaaagacagccatgatttacctgggttattttttgcattgcagaatggacatgcagatagtattcgtatgttcggaagtctgcttaataagaaaatgcttagtagtgaacaaattaaagaattattgaaagtaaagcatgggttgtttatggcacttcaaaatgggcacacaaaggccattatggcgtatggagatatactaaaaatactcccccctcaccaggaatatatagatgaattattatggataaaaaatcccaacggtacatcaggactttttatggcattttataatggccataccgaaacaatacgtgccttttgtaatattctaaaaaactatagttttaccacaagacgattggtcgaaatgctgtccgcaactaataaagatggtattcctggtgtatttgtatctgtagtaaatagagacaaagaaacaatattagagtattgtagaataataaaagagaacaatctggaaccggatactatagcagaacaattttcaaagaagatgaaaaaaacgtttattgaaataataaatagatttaatcattttttatgagactgtaatttaaattgtgtaattacctgtttttgatatgttcactccaacaaggtgtagtgaccgggtaactgccaactttcaggcaaccacctcgattagcaaccttatttccatatcgagttcaaatatcccgtaggggtttacatgggcgtaaactgatgattgttcaggtttccatggtgtggccaaatcttcttcatggataaaggtcacatccagagggtgagtatgttccaatatttaatactaaagatatgaggcttggaattgggttgcatcttattgattttataagaaagagtaaagatcaaggattccgagagttttgttataacaaaaacattgatcctgttagtctggatagaattataaactttgtgtttcagcttgagtatcatataccaagaatgctaagtacagataacttcaaaaaaataaaactcagagacatatctttggaggatgcgattaaagcatctaattatgaagagattaacaataaagtgactgataaaaaaatggctcatcaggcccttgcttattctttaggcaataaaaaagctgatatagcactttatttactatctaaatttaattttacaaaacaagatgtcgcagagatggagaaaatgaacaacaatagatattgtaatctgtatgatgttgaatacttacttagtaaagatggtgccaactataaagtgctaaatagctgcgcttaataccgctacacttttgccagcccatgtttgcctccggggaatgggctgacggtttccataaaatggcgaacttttttcaacagttgccacattgagctgcactgatgattacgcgttattgtgtcgtgaagtgcctgccatagccgttcaacatgattcatccatggcgagtaaaccggctgataaatgaccctgaacttcgggttctccttcagccagctctgtgtttcccggcttttgtggataatgtagttgtccacgatcagcgtgatggttttcgcccgacggtatgtcgctttaagccgcttcagcaggctgatgaacagcgccgaacttttgctgttgccgcccacacagctgactttacctgtcccgctgtgcagcgctccggccagataatatttttcattctgtcccggcgtgaccacccgtttttgctgtccgcgcagttgccagtccgcaccgattttgggattaagatggatatccacttcatcttcataaaagaccggatgctctgcgctgcattcgtccagtgctttatggattgctgccatcttttcatctttatgcgggtcacggatacgcagagttggcgcagcccttcgccacacaatccccgcagacggcaaccagcggcgaacggttccggcatttaactggcaaccggttatctcattgatttttattgccagcagttctgtactccagcgtgaacgctggtagccaaagtcgccgggagaatgttttaccagctcacgtaacagtgtgcagatatgctcaaacggccagcgacgggcacgcccggcaggtaatgatttcagtccctcaacacccgactgcgtgaaccagttaatccagcgtccaacagaggaacgggcgcagcagagcgttctggcaacgtcgctgacacggtcgccccggtgcagcatcagcatggcagtcagtctgcgggcataatttttatcgtgtgttttatggatggctttctgcatcaggcgtcgttcgtcacgggaaattggtgctatgatcggcattgctcagtccggttggtgatttgttttgatttggcgattgatcagatcgcacaatccgggctaagttccctcaaagtgatctactattccgcgcagctatttagaatattttatcaataatggattggttgatgtaaacaaaaaatttcaaaaagcaaatagtggggacactatgcttgacaatgcaatgaaaagcaaagattcaaaaatgattgattttttattaaaaaatggagcggtatcaggcaaacgatttgagaggtgacctaaaatatatttaggttgtgccctgaatctgaagttgtcatttctgttatattgatcgacataggggatggtgcagaccgttcccctgcaacaccacattgcatgtggtgaacggaaccgccccgagtttcctagagaatgttttatctgtgaactcaggttgccagatcatcgtttccgatggcagcataataagctttttctgcttctgccggagggatatgtcccagcctttccagtaatcccttaaattcaggggtaaaacgtggtgtgcccatagactcctcctatgctcatatagggtagatttgtctatgggccctccatagcctgatacaggctgtccagctttatggggtcagttcattggtaggagtatttaattcttcaaactcttttttggcttcgctaaggcttttggtaaattcaggtgtattatgaagtactgcaacaaccgatgctcccattaaacgcccagcctctacatcgctttgccaatgcgcaccgcagatgacccgactttctccaaactcatatccacgtcgaagtatttccgctttacgttgaggattaatctccgcaagtatcagtgctactgcccaaccaaaggatgcatgaccagagggataagagccagtgatagccatgttctcatctttatcaggtgtacaggttgcgtctttataaataacgaatggacgaactcttttataatattctttggcgttacgcacggcgtaatcatggctatcctgtagtacttgtgttaacaactgatataagattggggtattttcccttgaaatatccatgccataagcatctgaaaatgcaagaccaaaattctcgtagtatgcatcttcgctagcgagacgtacacgattagcatcccggagcgagcggccgaattcataatgagctttgtcagcctgaaatactactgaatcctctgccggaggcggcggaagtattgacaaactgtctggtgaagtttgttgagtgagaaaaccttctgccttcagagcatttgctgaggggataaaaatcatatttgtagcaatacaaaaaagaagaaagtttttggttttcataatttatttccttagcataattaattgatgattttggtcagctgtaaaaaacgagctgtgttacaaaaaacacaagtcatgcggaaaaatttgaacagagtgatggaaaaacaaaaaatagtcttatattaaagtaataacattatttttcatattcacgaaaaataatgtggtaaaagaatgatgttttttatctatttgttcgtatcgttttgtctctgtattatgatgatttgtgattttccctcatagggaagcacttttctactaaagataaaataacttcatcttaaatgcaaacaattaactataggttaaaaattttatactaatccagttctttattaataataaaagtctttttctgagagcttctttttactgtatgctccggatcagcaggaattgaagttgaacgataatgtgagtagggaaaaagctcttgaccatagtgaactccatatccatgatagccggatatttttagaccatcactcaatgactcatcgttttcaaggtgtttttttatctgctctagcaaagattctttttccttaaaaaaaggcagagagttaaggatacaagaaagactttcagcaggggcattgttaaaatttttaggagccactttatcagcggagccgcatgaagtaaaacggatatctttcacatttagaataccacagtctttcattcttgaaatgattgtttgtacatcaataagttccgaaccaaggcctaattgatgagaaccaggactaccgtgcccaagaatataaaccttattatactctttaaaatcagtatcgtttactgtttttattttcgctttttttacagaaaaataagcagcagcattcaatgcgtcatcatcattatttattactattggttcactctgtggtagccatgattcagccatttttagaaaatcatttttacttttttcatttttaaaaacataatcatataacatcttgttcttaaatactcttggtatttgttgtcccataatagatagaacatcattgctatcacaaaaacctttctctcctaaaaaaaccaatgctgcttttgggtgagtacatgatgaattttttaccctttttattatatgtgcaaatgcaatatctgatatgctttcattctttttattgactattctacagtatggtctaacaccatctaaattcatacctacatgactccatatattgttttataaacatcatagtaaagggtgttatattttcatatagtatgcaagcttctttgtatactatgatttaaaaaaacggaacaaaaatgcaattatattctggcattgtttaatgctgtttgtcttttttattcattatttcaatgaaagaatttcaactacaaagttaattgatcctacctacataatatagacacagtacctaaagtaccgtggagatcatccggggaagccccgggtctgataccagtgcaggtagaccgctatcttgttccggtaatgttctgccgttctttacggcatcacctctagcagaaacatcgcgtaactgcgccaagtatgatgaacttctcttaccgctgcgatctggttcaacaatatgcagatggtacacaacctctttaaccagcccatccggattattcagagccgcttccgcaatccggaataaaagtgctgttttaccgtgaaccctctgaaaatcagcgagcagtgttttaaccagccgacgttcagagcgtactgacagacggtggacaatttgaataaacagttcaatcagaccgtagatgctctcccgttrgmracmtrctggrarcrrtrgaamammgcttcrgcaamgagcttccggcgtctccagtgaagtgactgacggataatggttcatgctaccgggctaatgaaacacgccagttcacccggatgttgggactttgttgaaccgamgmacacggcggtgcggagtccggagagtaacagaatagcaaagagcttcgtgaaaacaataaagtgtgactacatcagtatcatgcccaaacctgacgggttaacggcagcaaagaaccttgcagaggcattcgagcattataacgaatagcatccgcatagtgcgctggattatcgctcgccacgggaatatctgcggcagcgggccaatgataacaggtgtctggaaatataggggcaaagccacccacgaacgatccagcatataatcaagaaattagatttcatttaacataagatagattatgtgcaccaagaggcgtttccttaatttctgaatttacaggctcttttgctctctttgctatgctgtggatttggaaagatagagtgctggctgattatttaggtattttcatcagcat

SEQ ID NO: 34 - DNA, 215774 bp, Artificial Sequence
  pINV_53G
gtccctgatagcactgttccatcagcgctttcaggcgataatagtagaccttgggtagcctctgcccatgtgtaacttgataccccgagaacagccacgatgatctaagcggttcttatttctccggtctcagggtcggtaatgccaacagtaagtccacagtaatcaacgaagagttttttgaaccgccccggaaatcctggagactaaacgccctgagaaagaggtaaacagaatgactaaaaatacacgtttttcccccgaagtccgtcaacgggcagttcgtatggttctggaaagtcagggcgaatatgactcacaatgggcggcaatttttagccatgaaaacgtaatcaactcggtgaaccaattcaaaaaatatactttatacctgcggaagtgagaaaaccaatatattcaatacttgcactatttttaatggtgccagatatattggtactttgctcataaagagatgtatcaccttttttattagaaatgtaatttctggagccttctatataaaattttgcactgggggtgacatagtatccaatattcgcagcaacagagcagtaattttggttttttatttcatctttaaaaattgtctcagtctgatagtgtttatctgtatcagatgaacgaacccaaccactatatttcagttctgctccaaactcaaaattctcatggcggtaattagcagttaatccaatataaggtattttgaagagctgtttgtaatctattgttctttcaccactaggatgtgccccttttttattcctgttccccccgttttcactataaatataactccctcccattgcattaaaactgtaacgactctcttggtaacctgctattagtccaagtcgataatccaaattatttaataaccaacctttaagattcagctcgaattcgttagcatcacggagatgcgtattgggatgccagctctggtcggtccatacctgaggggtattggaattgttccagtctttatcaaccatgcttgctttctggttacctaaagtagtccaaccggaaactccgaaagacacttttggaattagcttccaatcgatgccacctctaacaatagtcgcattactgtatttccagtccagttgactaatttttctccccccttccttgggatgataaacgcgttcttttgttttgccactcagagatcctagactaatatccgtactgatgttgtccggtatgaaaagtggatagttagtggtagcatgtgtagtaaagatcgctggtacacaaagtgcaagtacaaggaattttaatttcatcgagaactctacttttttggttgaaatgtccatgatcttcttactttataagataaacgtcaatgccaagcattattttaattaaatatttgtttatgttaccaagaacatatcttttattataatatttattgaacttttgtaaaaataaaaaaaatacctcattgtgcgaataaagtaacgggggccatagatttctctacctaccttttggtgttcatacctgaggctgatagtttcaggattccgcgacaattctactcaccttcttgttctctcgccaatatgaaatcaatcagaaaagcgttccataagaacaacgataagacaataaattgtcatcacggctgcaatagccaacgtgcctttcagtaccaacatgacaacctcctgtatttgacgtaacgtgtgctgataaatatgtatttgtatctcaactgactcaatacaacacttaagtatagtttttcagattttgtttttcccgtgaaatattatggaaatggcagaagcaatacattgaattccgattcgagtgaatacggctagcgggcacctcactttagcactgaagccatcaacttttatttgacttatttcaatggaaagctttctcactatcagtacaggtaattcccatccttttaaagtcgagagtcagactgcgactaatacagttaaagtgaccaaactcagttatcctctacacattttacctcatgtttacttggacaattcattggtgcttttataaaagaataaatattctactaatttaatgcatcacctaaaatagtattttttataatagaactgtatctatagtatattattaaagcttaattatttattatgctttaatgtggttgtccgattaaggactaatccttgaaaacgccgtatagggcgggagttcactctattgcgataacgtaaccgtacaatattgtgcgtttcccaaattgaccactactgaactattctgggatttaaatgcaataaaatggttggttgaaggtcgtggtgatgatgttagtccttttgagaatattccagtgccagatcttacttataataaagaggcttggcagtttggataagctattgtccttattccactaatacaaactttttgtaaagtatattaaatatacttcatttcatactgaaatccccggaagtattcaatcaaacaattacaccataaaaggaagagggagccccaattaagttatcaagcatgtttatgatacctgcagggtataaattttctggctgcaagcagacacatataaaacgtctttattgatcctacccacataatatggacgcagtcgtaagtgaggctctgtgagcctacccacgtaatatggacacaggcctaagcgaggttctggttttcaaattgttccggactgaggccgccacaccaacggtgccgtcgccagcgattgtaatcgcactcgatataattaaacactgttgcccgcactatttcccgatagataaagtgttctccgtggagacattccacttaggaacaattatattgttcatggtaatgtttatatttgagtatagagattaaaatgattagtcccatcaagaatattaagaacgtatttccaataaatactgcaaatactgaatatatagtgagaaatatttatcctagagttgaacatggttactttaatgaatcaccaaacatatatgacaagaagtatatctctggtataaccagaagtatggcacaattaaaaatagaggagtttattaatgagaaatccagacgacttaattatatgaagacaatgtattctccatgtccggaagattttcagcctatttccagagacgaagccagtacaccggaaggaagctggttgacagttatatctggaaaacgtcctatggggcaattttctgtggatagtttgtaccatcctgacttacatgcattatgtgagctcccagagattagttgtaagatcttccctaaagaaaatagtgattttttgtatataattgttgtattcagaaatgattctccccaaggagaactacgggcaaatcggtttatagaactatatgatataaaaagagagattatgcaagtgttaagagatgagtcgccagagttaaagtctattaaatctgagattattattgctcgtgaaatgggagaactttttagttatgcatcagaggaaatagatagctacataaaacaaatgaatgacaggctttctcaaatcaaggctcgaatgcctgttacttaatgtatattaattgtatgaagtggtcaacaaaaactggccaccgagttagagtttttccagtatcgattttccgattcgtttgggggtaacccaccgttatattcgtgcggtcttagtgcgctgtaatatccaacgatatagtccgttatggcgtgagctgcctcgctgaagcttacgtaacccaccatcggcatccattcgttcttcagactcctgaagaagcgttccattgggctgttatcccagcagtttccgcgccggctcatactctgtctgatctggtatcgccacaataactgccggaactgcctgctcgtataatgactgccctgatcgctgtggaacatcaccccgccgggcttaccacgggtttcccatgccatttccagcgctttcatggtgagcctgctgtccggcgagaacgacatggcccagcccactggttttcttgcgaacaggtcgagaacaacggcgaggtacgcccagcgcttacccgtccagatataggtcacatcaccgcaccacacctgatttggctcggtcacggcgaactgcctttcaaggtagttagggatagcaacatgttcatgaccaccacgtttataccggtgagtcggctgctgacagctgaccagccccagctctttcatgagcctgccagcaagccagcgtcccatctggtagcctctccgggttgccattgtggcgatgcttcttgctccggccgaaccgtggctgatgccatgtagctcaagtacctgactgcgtaatacagcccgtctgccgtctggtttttcaggacggtttttccagtatctgtagctgctgcgatgaacccccgagtaggcagcctgacggctgcggcttgtcatggtctgcaattaccgttataaaaacaggcaatgtcattgtctttcaggtggttatatggcccgttcagctaaaccccgtaaacgtaaacccgcctctcaaagaagcaaacttccccgctatgtcgtgaagcttcacgaagatgatttttttgacgaagaagacgcagaagttctgcgctttgatagttttgacgatgccgttgaatgctgcgcagacctgaatattccgttctttgtggatgcgggaaacaaaaagctggtcttctggtttgtacgtgttgatgacgaagggtatccggaaatcgcccgctgcacggagcgggaatttgcgaccattctttccggtatcagcgccggcggtatgtactgcccggagtgtggcacggttcactggccggacggagtcgccccgcccttctgatgcttccccgttttgccgatatctttcagcagggaaaccgctggcttaactggctggagaagcagccggaaggggcagtacgtccggtggtgattgagtcggtgaccaaaatcatggcgtgcgggaccacactgatggggtatacacagtggtgctgttcatcaccggactgttgccatacaaaaaaggtctgcttccggtgtaaaagccgctcctgcccgcactgcggggtgaaggctggcgcacagtggatacagtatctgctgagcctggtccccgactgcccgtggcagcatattgtgttcacacttccctgccagtactggtccctggtgttccacaaccggtggttactgacagagatgagccgcattgcagcggatgtgatactggaaatctgccatcaggcagatgtggagccggggatattcacggtgatccacacatgggggcgtgaccagcagtggcatccgcatatccatttatcgacaactgccggtggtgtgacgtcgggccacacctggaaaaatcttcatttttacgcccgtaaggtgatgagcatgtggcgctaccggataacacggttgctgtcacggaaataccctgacctggtgatgccggatgcgctggcagcagaaggaagcagtaaacgggaatggaatcgcttcctggacacgcattaccgccgtggctggaatgtcaacgtatcccgggtgatggataatgccacccatgtggcggtgtactttggctcttacctgaaaaagccaccagtgccgatgagtcgtctggagcattatgccggtcaggatgaaatcggtctgcgttacaacagtcaccgaacaaaacgggaagaatacctgttgatgagtggagatgagttcatggaaaggttctcctggcatgtggcggataaggggttccgtatggtgaggtactacggttttttgagtccggcgaaacggcggttactggaagaagtggtgtacatcataacggagacagtgagaaaaacggcgatgcaaatcacctggagagggatgtatcagaggttactgaaggttgacccgctgaagtgcgtgctgtgcgggagtcagatgcgttttacggggctgaagcggggctaccgtctggcagagcaggtcctgatgcatgagctgctggcccggatgcggtggtgcggctgagagccgcagaggggaagttgcgtccattttgagggaaacggagcaaaaaaccgccattcataccctgaatcaatcagagccatccagtttaatcgtcggttccgttcatatggagcaaaagtggtgttgaagaaacatccgttttgtggtgtttttttaatctttttggggttttaattcctatcgatgaacgcaacctgacaagagagagaactcaggtcgggcttcaagcagcacgagccagaggacgtaagggcggcaggccgaaaacactgagtaaggataaacaggcccttgctgttcaactctataacgagaaaaaacatacagttgcgcagatttgtgtgttgatggggatatcgaggccaacgctgtacaaatacatagagtcagccaggttgttcaagaaatgaacttagcgtacgttttcgtacggttgggcttcaaatccctaatacgttgacggggcaatatgcagttcgctgcatagcggtccgaccccgtactgctcacgcagcttatccagcagtggcatcattttttccagaggcggtcgaactccgccttcgcaaaataagcggaagcttggcgaaggatatcgttactgcggcgcagttcacgattttcacgctccagctctttcagacgctgacgttcagcggtggtgagccctccatcaccgcccccggtatcccgctcatgctggcgaacccggacacgcagagtctccggcgtacagccaatctttggagcaatggaacaaattgtcgcccattgtgagtcatattcgccctgactttccagaaccatacggactgcccgttgacggacttcgggggaaaaacgagtatttttagtcatcctgtttacctctttctcaggaagtttagtctccaggattcccggggcggttcatttgaggatcgccatgatttggctgtcggtaaattttgatgttttcataaagaatctcctgcgtttatggtacgagaaaattctacttttgacgctggttaatttacggggtgattaccagaccaatcgccagtggcgcaaagcccgcaggagcgcgtttttctgtcgctccggacaaaaaacgcactgaggacaaactcgctcagcatacaggcgcttaacgcatagccgccaggtgaatggaggccataaccattagccgcaaaaccgctggttgtcgcatcaaacccagccttaccgctggcgataccatacagcactgccgcagcggcaataccgccgataacctgagcgataatgtaaggtaacacgcgcgctgcagggaagcgtccaccggcccacagacccaaggtcaccgcggggttaaaatgcgcaccagagatgtgcccgaccgcataagccatggtgactactgtcagaccaaacgccaacgccacgcccaaaaagcctatacctaactgtgggaaagcagcagagatcaaggcgctaccacagccacccagaaccagccagaaagtgccgaaaaattcggcgctaaaaggtttaaacatacactgatgtcctgtttgttaaagagcaggcatcagggtaaaggcggtcacgatgtaccgcctagcggggatttgtaagagaggtgaagaattggtgtggaaggttaaacctaagcaatcacaggcgaatgacattgctcgcttgtgataacagcgattttttctgctgccaaactagatcgcaaatatcatcagttggattgaatgccgctggtgcttgtaacaacaaactgcgaattcgctcatgatcaaagtcatgacaagcttgatccagctctttaagtaagagcgatagatcctgccacggtagcatcacttcgttggccgtcataattcgtggatgagaggtaccttgaactgaatcaccaatcagtaattcttcatacagtttctcacctggacgtaatcccgtaacttcaatggcaatatcgccatctggatttttatcatcccgtacactcaagccacttaaccggatcatgcgtttggctaagtcataaatcttgaccggatcgcccatgtctaagacaaagacatcgccgccatgccccatcgcccccgcttgaatcaccaactgtgatgcttccggaattgtcatgaaatagcgaataatgtcacgatgagtcaaggtaactggcccaccttgggcaatctgtttttcaaacaacggcacgacagagcccgaagaaccgagtacattaccaaatcgcaccatacaaaagcgagtttggctttgccgtgcagacaaagcctgcaataccaattcggccagacgcttagttgtccccatagtgttggtcgggcgcaccgctttatcggtggaaatcaacacaaaagtttcaacgccactttcgatcgctgactcagcgcagtacaaggtaccaaacacgttattacgtaccccttccaccacattatgctcaaccagaggcacatgtttataagcggccgcatgataaaccgtttgaataccaaaggacttcatcaccatctgtaagcgattctgacgctgcaccgagcccaacagagggatcactggaacattcaaaacttctttgtcacacagcgccgagagctctttctcaatagcgtagagggcatattcagacagttcaaatagaaccaatttggccggcttatatcgaacaatttgacgacaaagctcagagccgatcgagcctcccgccccagtgaccataacggctttgccagtaatgttttcggccatcaatttggcatcaggagcaaccggatcacgacctagtaagtcatcaatcgatactttttttagattactgatttgtgctcgaccttcgactaaatcgaccatacccggaatagagagaacttcacacggtagatgctctaaacgagtaatcactttctgcctttgtgacttactgacgctcggcatcgccaaaagaattttctttataccatagcgatctacaaggtaactcagtttatccgagggataaactgttacaccatgaatgacagccttatgcaagcgaggattatcatccacaaatgccacaggaaaatattcttttgcctgcattaatgctggcaataattgtcggccagattcaccagcgccataaatcaaaacaggcgcactacccttaactccatagttaagtatcattctaaaaagcaatctagagcctgatgtcagtaaaagtaaaaccaaaaaataaattccgacaacagtgcgcggcaacattatggatagcgaatatgatataacgacaagagataacgttgaggccagaataccaactggtattagcaacattatctttgcactaacataccgtaagactgcccgataaagcccaatcctgataaatataaacacggtaaatatggtaagaatagctgcagcaaaccacatcggtgcactaagaaaagccgtttgctcatcaagcctcaaccaataagacaaccaaaatgagaatatcaccataactacatcaatgcagatgatgatacctctcttaacaattcttggcagctccaatattctatcaatcatcctacactaccttattatcgcgacaaaatcttaataatagttttccaaattatcacacaatcatactttactgaattgttagcaacataatctaaataatatctttgcttgattggaagaattatatctatataagccctacgtgggtcatcataactagacaaaatctcattttcatctaccatttctatagatgctaagtcagttatccctggcctaaccgataaaactttttcccttatatcatcaggatactcatcaataaactccctcacttccggtcttgggccaaccaaactcattgttccagaaagaacatctatcaattgaggaagctcatcgattttgtacttccgtaaataccatccaactctggttacccgagcatcttgaccaactgttatccgtccttttttttctgaatcgataaacatcgttctaaatttatgtatcctaaaatctttcccaaatctcccaactctatactgcctaaaaaaaaccccccctttagaatcagcaacaattaacattgacacaatgataaaaacaggaaatagaaaaagcaggcctaagcctgccacgataacatcaaaaatacgtttcatttaatataaaataccattacataaataattgtctaatcgatttgataacgaacaactgatcttgatccgtcattttagtataaagaggaatagatatttcatttaaataacactcctcagaaactggaaagtcactggcgttcaaattatatgtatcacgccaaaccggttgcttatgcaacggtataaaatggacagaacaaccaattccaagatctgataacttcttaataaaatcatcgcgattaatagccgaatcagtttttaagcggataggatatagatgccaagcatgaatatcactagcattagtaggccattcaggcaattcaagtggcaattcttttaacgcatcatcgtaaatttttgccattcgttgacgttttttctgaaaatcatcgatctttctaagttgatgaataccgattgccgcacagatatcaggcatattgtatttaaaccctggagctacaacctcataaaaccaagaaggagttttagactggtaccggtcaaaagcgtcacgactgattccatgtaaacgcattaccttacaacgctcaattatatctttattttttgaaacaaccattccgccttcaccggttgtcatagttttattggcgtagaagctaaaaaccgtagcatctgaatcaagcgttcctatcttacttcctttatatgttgtaggaaatgcatgagcggcatcctcgacaacctttaggtcatattttttagcaattgataagattgaatccatgtcacaagctaatccagcgaagtgtactggaataatcgcctttgtttcattagtaatagccttctcaatggcatcaactgatatattaaatgttttacgatctacatcaacaattacaggatcagcaccaaggtacctgacaatttcggcagtagcagtgaatgtatatgatgggacaataacttggtctcccggctttacgccaacagcttcaagagccaaatgtaagcccgacgtagcagagttaacagctaatgattgaacgttcgctcctaggtaattagaaaattcttgttcaaattgcttagccttaggacctgtcgtaatccaacctgaacgtaaagagtcaattacctctgcaatttcttcttcgccaatttcaggtaacgcaaaaggaataaaatttttcatttaaaataccttttcaaaaaagataataacgatccacatccataagaaatatgataagacaaaaatataaaaggaagaaatactctacctataaaacttcgcccatctcgaattgaaaacaaaatagaaataagaaaataaaaaaataaaaaagataaatatatataatcaccagtagcaaagaaaagagaaaaagatacaattaaatacaaaacaaaaaaaagtggcactatatgtctaaaataatatgctccagacataacaacccaaaaaccatcaagaaaacctttctttatgaaatctttaaaattatctctaacataatactcaacaaataaactattatctgtatataatgacaaattattgcttaaaactctcttatgaaaatcaatatcttgatttctatctaatacttcattaaaaagtccaacatcaaaaaacacatctttattatataaagcaaagacagccgtatctgttttttttagtcgattatttttgtctacgcacctaaatggagaattaccaacaccaaatttcgatgcataaaaatccgctacaattgaaccaaacttagttttataagaattagttaatacacctccaacaccaacaacgctatcatcaaactgcataatattaaaataatcatctaataatttagatatatagttatttggaatatcactatgtgcatcaactctacacacaaacttaccattagcttctagcaccccaatattccaacccgtagcaagagttctttttttattattgataattttatgcttaatgttgctatgtttagaaaaaacatcctgaacaatagatattgtattatcagatgaatccccatctacaataatcaattcaaatttatctaatccaacatcttgattaataaatgccaaaagagatttttcaatatacttctctgcattaaagcaaactataattaaagatactagaacatcactcacaaataccactcctcataaaatatagttttcttttccctgagtgaactgaatcaacaaacaaaaatctattatcaaccgagatccttggatgtaagtcacatcgagaataagaaaaaaaactcattgactcataaaataatccaagctcgcgataatcatttttgacaaggtcataaacaaacaactttttaattctatttttatctgggtaagtatcagatataatatatctattatgaactatagtaggatgtccatcagaatattttcttgcatcaaaaaacaatttctctgtattacaggattcaattgacaatctataataatttagcgaatctatttctgcaccaaaaaaacctataatttcatcattcccattccaacaacaatgactaataatcgattcatttattaggactcgaacattatcatttattaaattccaatgtattaacctttcacgctttcctttaggtgtatagtatcgatgtatgaacataacggaacgtccatcgggagcaaacataaaatgattgaccacatgttcaacatcaatattacatttaaaattagttctctttagcatttcgtctaaggataaaaataattctattccccccgtgttcaaattaatgattgaaatatctgtagaaaatgattttttattataataaccataatcgctatcatatttagccagccatgagaaatctaacgtcacagccttgtaattattcacatcataaatcggatagtttattttttttataaccttcatcaaagacaaagaatagacaacactaatgtatccattattttcatagtcattaaaaattatattgtcatcatcaatccacattaatcggcttccctgctgccaattaaatgctcgcgtatcatatagcttataaactttgttattaagaaggtcttttacgcaaatagatatatacttaactttctttggatgtcgtttagtgctaaacgcactagagtggaacaatacgtaccgcccattagagctcattggactatggtcataatagccaaaaaatgattcttcagaatcatcaattgaaatctcgtatatttttgattgaataaccgtcttatttttattcaaaataattccgaataatgatgcgatatataaataaaagaacttaataacatcccttaccttaggatacttactcaaggctaaggaaatttttctttcaacatattctaacaacatattctcatcctaaatagctgcgcttaataccgctacacttttgccagcccatgtttgcctccggggaatgggctgacggtttccataaaatggcgaacttttttcaacagttgccacattgagctgcactgatgattacgcgttattgtgtcgtgaagtgcctgccatagccgttcaacatgattcatccatggcgagtaaaccggctgataaatgaccctgaacttcgggttctccttcagccagctctgtgtttcccggcttttgtggataatgtagttgtccacgatcagcgtgatggttttcgcccgacggtatgtcgctttaagccgcttcagcaggctgatgaacagcgccgaacttttgctgttgccgcccacacagctgactttacctgtcccgctgtgcagcgctccggccagataatatttttcattctgtcccggcgtgaccacccgtttttgctgtccgcgcagttgccagtccgcaccgattttgggattaagatggatatccacttcatcttcataaaagaccggatgctctgcgctgcattcgtccagtgctttatggattgctgccatcttttcatctttatgcgggtcacggatacgcagagttggcgcagcccttcgccacacaatccccgcagacggcaaccagcggcgaacggttccggcatttaactggcaaccggttatctcattgatttttattgccagcagttctgtactccagcgtgaacgctggtagccaaagtcgccgggagaatgttttaccagctcacgtaacagtgtgcagatatgctcaaacggccagcgacgggcacgcccggcaggtaatgatttcagtccctcaacacccgactgcgtgaaccagttaatccagcgtccaacagaggaacgggcgcagcagagcgttctggcaacgtcgctgacacggtcgccccggtgcagcatcagcatggcagtcagtctgcgggcataatttttatcgtgtgttttatggatggctttctgcatcaggcgtcgttcgtcacgggaaattggtgctatgatcggcattgctcagtccggttggtgatttgttttgatttggcgattgatcagatcgcacaatccgggctaagttccctcaaagtgatctactattccgcgcagctatttagaattacgtataaaaaccaccgataaaaaaatagaaatataaatatatatcgctggcgtcaatatatactcattaaaaaagacatagaaagccaaagcaaaaacgcatcctttaccaatataccctccgcatttaaaaattggagacagcattatataaaatacagaaataagcaaaataaaaccaataatccctaactgagccgaataagataaataaaaattatgcgccaacccaaagttggtacttcctaaaaactttttatctatagcctttacataatttgcaacacctaatccagtgccaattaaataattttcttttaaaacatcaaccccaataaatactaactcaactcgacgataatcattctgactttttgtaagagtgttaccaccagagaatataacctgtgacgaagacagcattcgttcaagacgagtgcctatatcagtaaaagaaatacctaaagttagaagaggaacaaaaaacaatgatattaataacttttccttctttgttaactgaagaaaaacaaaaaacaacgatatagcaaatgatattattcctgctcggctatccagaaaaagaaagaacagagagtacaatacagatgagacataccaagactttcgaccacgataaaagtatgaatatacacaataaaagcacactactataattgcggtggcatttctatttaaataaactataaaattactatcttcagaataaccaataaataattttacaactccacagataataccaaaacacattacaacatttatatttctaaccgaatgagttaaataaagctcatctttaccaatgcaaaatgacaccaaaagaaccaaagctataaaattagaaaaaaacgataataaaattacgacacctgatatatccgctgataacaacaacatctgaatgctgatataaatcaggaatatgatgcctaaaaataataatttctttgtccacagagaaacgtgaaacctcaacgttattatagatgttaaaacaacaaagaaataaaatattagatctggaatatagccaataggaactccaaaaaaacgctctaaatcagagttcatttgtccataaaaaaaaaccggaaggatattaaataacaaaagtacaggatataggtatattaacatacttaacctttattaggcttccaatcgagtctaaacatattaatggaacgatataaaaccacaacatattaatataacaaccgctgctgaccattaataataaaataaaagaaaattgggacaaagatcatcattagtgaatacttatcatgaagaattttcccacaatcttttttcacagaaataatataaaatattccaattactaatgttgcaatggcacttccaaaaaccgccccactccatccataccgaggtataaaataaaagaaaaccgccattgaaatgaccacaccaatacaatacgatttaactcgctcgctttgatttccactacacacaagataaagttcatttattagccaaaaattatatttaataagagccataaacattatcaaaaatgttgcagaagatattaccttatactcatcaccaaacaatatccctacgaaatagatgccaaaaaaataatagcataaacatagtaataagactaacagtgatatatatcgttgtaatttccttatattaccccattcagaatttttcagataataagaaaatgaaggcaataaaacagtataaataacattaaacactattattagaccggtattaaccctagttaccacctgatatatagcaacactattataagagaaatattcttttaatataaaaaggtcaatactagataaaacaacatttagtaacaccagaatagcgaacggagttgcatccaaaaaatctttataaactaaaatacttggtctttttatatgaaaacatggcacattgcatttatgtgatgagaaaaccaaaagcgaaattgagtatatgaatattgatatcgcactataatatagaggttcgataaacagagataaaaataaattacacccaatatatataattgttgagtagatatttatatataattcacatctaaaccgaccaacagcctgtagcaagctaaatctaccacttgaaatacaaacaaaatatgcagatgaaatataaacaagcagagttaatgaaattgaatcagaaacataagaataaaacatgaagattataaaaatgaagaaaaaaacatatgttttaactccatcacttcttgataataattctgatgcagaataactattatcagaaatatctttcactaattttgttctaaatccataatcagagaagttaagcacaaaccaataaacagtaaacacataactaataataccaaatgtagtaatatctgacacatgtgtgaaataaacaaaaactcctatttgaaatattgctttaagtaaaaatgtaccacctgcatcaatcatttttctaaacatacactgaccatttagaataaatacatggattttcacccctgcattaataacagaggtgaaatagattatttattcatgttaatataatttagccttttaaaaatctcacataccacggcattgaaagtcgtaatccctctctgatttttatatttggtctatactttagtaaatctatagccttagtaacatcagcctgagaatgcctaacatctccagatctaaactctctatacttaatgctcaatttatcgatatggtgaattaaattaagctcatcataaatgtaaccagataattcatttaacgttgttctatcaccaactgcaacattatatatattatctttagcactgtccttcgctaatgcagataatatattcatttgtataacattatctatataacaaaaatcacgactcgtttcaccatcgccattaatatatacgtcatcacctttaagcattgctgctgtccattttggaattactgcagcataagctccattaggatcttgacgacgaccaaatacattaaaataacgtaatccaatagttttaaaaccatatgttcgagcatatacctgagcataaatctcgttaacatatttagtaactgcataaggagaaagtggattaccaatgttttcctctacttttggtagtgcgggatgatctccataagttgagcttgatgcagcataagtaaaactttgtacttgtgcatttttagccgcatgtaagatattcaaaaatccagtaatattagttgcattggttgttataggatcaacaattgaacgaggtacagaacctagcgcagcctgatgtaagacatgatcaacacctttcataacttgctcacaggtagtgagatctcgaatatcaccttctataaagcaaaatcgactccactgttcagtggaaactaatgttttaacttcatcaagattatattgatgtcccgtggaaaagttatctaacccaataacaacctggtttaatttaagtaacttttctaaaagatttgatcctataaagccagcgacaccagtaattaaccaagttttcggtgaaaaaattaactgctgtgtaatctcttcatagcgagacatataaatatccatgtatattaatcaagtgctaaacctagcataaaatgctgaggtgtatcatatcaaagaggatttgatttttttaatcactcttacaagcgcatatcgatactttttttatcaagcacatattttaaatcgaacaaaacgtgctcgtctttacctaatgcacgaatagcgctctctcccatctcgcgaaactcattgtgtgcaacagcgataattattgcatcataatgattaactttaggatcttcacataaagttaatccatattcatgttgtgcctcatcggtagaacaccatggatctataatatctatattgatattgtattcttttaactctgaaataatatcaatcactttagtgtttcgtagatctgggcaattctctttaaatgtaagccccatcactaacacattcgccccttcaacttgaatccgttgtttcaacatttttttgactaactgggaaacgacatactgccccatactatcatttaaacgacgtccggctaaaatcatctccggatgatagccgacactttgcgctttatgtgtaagataataaggatctacacctatacagtggccacctactaaaccgggcctaaaaggtaaaaaattccacttcgtacctgcagcctcaagaacctctaaggtatcaatccctaacttattaaatataatagataactcattaatcaatgcaatattgacatctcgctgcgtgttttcaattacttttgcagcctctgctacttttatcgatgatgctttatgcgtaccgacagtaattattaatttatatagctgatctacatactcagcaacatccggtgtagatccactggtcaccttaaggatattagttacacgatgctctttatccccaggattaatacgctcaggtgaataaccggcaaaaaaatcaatattaaacttaagacctgatactttctctacaactggtatacaatcttcttctgtcgctccagggtaaactgttgactcataaataataacatcgcctttctttattatcttacccaatgtttcagatgctttaattagaggtgttagatctggctgtttatgtttatcaattggagttggaacagttacaataaatacattacactcttttagtgcatctaatgaacaggcgtacgtcaatttagttgcttctaacaactctaaatttgagcactctaatgtactatcgtgaccatttcgtaattcatcaatacgagacttattaatatcaaatccaatcgtcgttactttctttccaaactcaacagcaagaggcaatccaacataaccaaggcctataatcccaattttcgcattcaaagtatcgaatttcattttcttatccatcataaagtgcacatagaacatctatttcgcattgacacaacaatacgtaacccagtttaatctaacttggttttatcttttaatgtcgcataaactaatgtaccaactatagcaaacattgcccctggtaatgcaccaagaatcaaaattaatgctttgttgggtttgtcttttataactggctcagatggcttcatttgatatcgatatgcttgcgcctgaatgttatcgcgtaacattccttctaactcatcaatttgacgtttcacttcgtaatatctaaatgggtaaataattggcgcatctttagttgcttttagctctgctgataacagtttttcgccgagcaagaacttatagctactttgcgcattatcgaagccgcctttgctagagcgataatcaatgattccggcttgttccgcagttgttaaatctcgctgaatatgagctatttgatcaagcttttctgattttctctggaaatctatgtcaataatttctttatttagattaaccaaaagagtattaacattttgaccaaactcatcaagacttaatgttacagcttgattattaacattctttagatatccctgcagtagtttatatgcctcttgcgctgagtctgccgatgcttgtattatgtagtaacttgtatttcccttttcctttggtggcgtaatcacaagacctttattcgcttgctcagctaaccaaattttcttatcttgttcagtagataaattatttgtttcctttttataatactcagtactctctagaaatttaactttggtgtctggcgattcagcaatcaaaatcaaatcggaaaacaatccattagccaacgcattcgtatctaacggttgatttaatattcgggcatacgcttggtgaaatttaagatagctgcctaattcagcaatacgtggtgcttgaatataggttttagccgtccactgctcttttgcggtaaaggcatacactgctgctaaccccgtagtcagcaacgtgacacataaaataacccattttttcttccacaaggtgcctaaaagctcgaaaagatcgatttcatcctctgcttttggcatataagttggatagaccccttgcgggatcagataaggggtctgttgtggttcagatgcttttgacatgaaaccaatccattatttatcaatgggttaaaaataaaaatgaagaataagcctgacaaaaaagtacgctaccgcccttgagtttcagctcccaaagcgctgcgcagaaacaggatatagccaactgcgatacaacaagtgatatctgggattagaggacataatcgagaacactccgtgtgaagagacatatacagtcccacacttccctgctgcaaaccggcggatagcccgcagaatttggagtgcagattataaagcaatgcctgtataagaccaaatctgccttcaaaatgataacagtatgtttgtaaattctgtgaggctaaatagctgcgcttaataccgctacacttttgccagcccatgtttgcctccggggaatgggctgacggtttccataaaatggcgaacttttttcaacagttgccacattgagctgcactgatgattacgcgttattgtgtcgtgaagtgcctgccatagccgttcaacatgattcacccatggcgagtaaaccggctgataaatgaccctgaacttcgggttctccttcagccagctctgtgtttcccggcttttgtggataatgtagttgtccacgatcagcgtgatggttttcgcccgacggtatgtcgctttaagccgcttcagcaggctgatcgagtaggcagcctgacggctgcggcttgtcatggtctgcaattaccgttataaaaacaggcaatgtcattgtctttcaggtggttatatggcccgttcagctaaaccccgtaaacgtaaacccgcctctcaaagaagcaaacttccccgctatgtcgtgaagcttcacgaagatgatttttttgacgaagaagacgcagaagttctgcgctttgatagttttgacgatgccgttgaatgctgcgcagacctgaatattccgtgaggtagcctgagtttaacggacactccttcctgaaatagaatggcatcagaaggagctaataatgagcagaaaaacccaacgttactctaaagagttcaaagccgaagctgtcagaacagttcctgaaaatcaactttcgatcagtgaaggcgcttcccgattatctcttcctgaaggcactttaggacaatgggttaccgccgccagaaaagggctcggtactcctggttcccgcacggtggctgaactggaatctgaaattctgcaactgcgtaaggcgttaaatgaagctcgccttgagcgagatatattaaaaaaagcaacagcgtattttgcacaggagtcgctgaaaaatacgcgttaatcgaacaatggcgacaacaatttcccattgaagcgatgtgtcaggtatttggtgtatccaggagcggttattacaactgggtacagcatgaaccctcagacagaaaacaaagtgatgagcggctaaaactggagattaaggtggcacatatccgcactcgcgaaacatatggaacccggcggctccagacggagctggcagagaatggcatcatcgttggtcgtgaccgactggcacgtcttcgtaaggagctaaggctacgctgtaagcagaaacgcaagttcagagcgactacgaactcgaaccacaatctgccagttgcgccaaatctgctgaaccagacgttcactcctacagcaccaaatcaggtctgggtggcggacctgacgtatgttgccacacaggagggatggttgtacctcgctggcatcaaagatgtttatacgtgcgaaattgtcggctacgccatgggagagcgcatgacaaaagagctgacaggtaaagccctgtttatggcgctcaggagccagcgcccacctgccgggctaatccaccactctgatcgaggttcacagtactgcgcatacgattaccgggtcatacaggagcagtctggtctgaaaacatcaatgtcgcgtaaaggtaactgttacgacaacgctccgatggaaagcttctggggaacgctgaaaaatgagagcctgagccactatcgttttaataaccgggatgaagccatctcagtaatacgggaatacattgagattttctacaatcgtcagcgtcgtcactctcgtctggggaatatctccccggcagccttcagggaaaaatatcatcagatggctgcttaaaaaaagaacaaatggtagtgtccgctattgccagtacacctcaccgttctttgtggatgcgggaaacaaaaagctggtcttctggtttgtacgtgttgatgacgaagggtatccggaaatcgcccgctgcacggagcgggaatttgcgaccattctttccggtatcagcgccggcggtatgtactgcccggagtgtggcacggttcactggccggacggagtcgccccgcccttctgatgcttccccgttttgccgatatctttcagcagggaaaccgctggcttaactggctggagaagcagccggaaggggcagtacgtccggtggtgattgagtcggtgaccaaaatcatggcgtgcgggaccacactgatggggtatacacagtggtgctgttcatcaccggactgttgccatacaaaaaaggtctgcttccggtgtaaaagccgctcctgcccgcactgcggggtgaaggctggcgcacagtggatacagtatctgctgagcctggtccccgactgcccgtggcagcatattgtgttcacacttccctgccagtactggtccctggtgttccacaaccggtggttactgacagagatgagccgcattgcagcggatgtgatactggaaatctgccatcaggcagatgtggagtcggggatattcacggtgatccacacatgggggcgtgaccagcagtggcatccgcatatccatttatcgacaactgccggtggtgtgacgtcgggccacacctggaaaaatcttcatttttacgcccgtaaggtgatgagcatgtggcgctaccggataacacggttgctgtcacggaaataccctgacctggtgatgccggatgcgctggcagcagaaggaagcagtaaacgggaatggaatcgcttcctggacacgcattaccgccgtggctggaatgtcaacgtatcccgggtgatggataatgccacccatgtggcggtgtactttggctcttacctgaaaaagccaccagtgccgatgagtcgtctggagcattatgccggtcaggatgaaatcggtctgcgttacaacagtcaccgaacaaaacgggaagaatacctgttgatgagtggagatgagttcatggaaaggttctcctggcatgtggcggataaggggttccgtatggtgaggtactacggttttttgagtccggcgaaacggcggttactggaagaagtggtgtacatcataacggagacagtgagaaaaacggcgatgcaaatcacctggagagggatgtatcagaggttactgaaggttgacccgctgaagtgcgtgctgtgcgggagtcagatgcgttttacggggctgaagcgaggctaccgtctggcagagcaggtcctgatgcatgagctgctggcccggatgcggtggtgcggctgagagccgcagaggggaagttgcgtccattttgagggaaacggagcaaaaaaccgccattcataccctgaatcaatcagagccatccagtttaatcgtcggttccgttcatatggagcaaaagtggtgttgaagaaacatccgttttgtggtgtttttttaatctttttggggttttaattcctatcgatcaagcggctggcacgtaaaacaatctgcttctcgcgttccgtggaaatccacgaaaagtcatcggctccttcattgaaaaacacatattctactgattggaagcgccacccgaaaaagtatcgtttttttacagtaaacttcatttagccgactataatgtaaaaaaataaattttttaccaaaatataacataaggaagtaatatgcttacacaaactatattcccttgtcttcccaaaaaaataagaaaatatcatccctgaagtatcaaatactatattattatcttctactgttactacagatggatatactgtttttaataaaaaaaacagccatatacgaattgcaaatatctgctaccaaccgaacgaaaaggttgaaattcacagctacagaaatacaatggttgacaaagataaatgaagccggaattgataaaaaacaaagccaacggtattctgatttctgaagttaatttcagactcgtttaagctctattttcaaaaaaaatatatcagggtttgttactaataacttctgaaaacagaaaaaaaatagagcagtaattctatttcagagaggagtaactccatctatcctcgatacgtactgaacgttatcgtgatatattcatcatgctttcttcaccattattcgagtacagggagagacgcaatgaccttaaatacctttttatggctgaattaccattctcaggaataacagcagactccttcccatagacggcttagggggaaagcaatacccggagaaagggtacaaagcaaactgttttaaaattaataagctatattctatttcactagaattcttatcattgataagaaattttagttataatgattaaatcaaccaatatacaggcaatcggttctggtattatgcatcaaataaacaatatatactcgttaactccattttctttacctatggaactgactccatcttgtaatgaattttatttaaaagcctggagtgaatgggaaaagaacggtaccccaggcgagcaacgcaatatcgccttcaataggctgaaaatatgtttacaaaatcaagaggcagaattaaatttatctgagttagatttaaaaacattaccagatttaccgcctcagataacaacactggaaataagaaaaaacctattaacacatctccctgatttaccaccaatgcttaaggtaatacatgctcaatttaatcaactggaaagcttacctgccttacccgagacgttagaagagcttaatgcgggtgataacaagataaaagaattaccatttcttcctgaaaatctaactcatttacgggttcataataaccgattgcatattctgccactattgccaccggaactaaaattactggtagtttctggaaacagattagacagcattcccccctttccagataagcttgaagggctggctctggctaataattttatagaacaactaccggaattaccttttagtatgaacagggctgtgctaatgaataataatctgacaacacttccggaaagtgtcctgagattagctcagaatgccttcgtaaatgttgcaggtaatccattgtctggccataccatgcgtacactacaacaaataaccaccggaccagattattctggtcctcgaatatttttctctatgggaaattctgccacaatttccgctccagaacactccctggctgatgccgtgacagcatggttcccggaaaacaaacaatctgatgtatcacagatatggcatgcttttgaacatgaagagcatgccaacaccttttccgcgttccttgaccgcctttccgataccgtctctgcacgcaatacctccggattccgtgaacaggtcgctgcatggctggaaaaactcagtgcctctgcggagcttcgacagcagtctttcgctgttgctgctgatgccactgagagctgtgaggaccgtgtcgcgctcacatggaacaatctccggaaaaccctcctggtccatcaggcatcagaaggccttttcgataatgataccggcgctctgctctccctgggcagggaaatgttccgcctcgaaattctggaggacattgcccgggataaagtcagaactctccattttgtggatgagatagaagtctacctggccttccagaccatgctcgcagagaaacttcagctctccactgccgtgaaggaaatgcgtttctatggcgtgtcgggagtgacagcaaatgacctccgcactgccgaagccatggtcagaagccgtgaagagaatgaatttacggactggttctccctctggggaccatggcatgctgtactgaagcgtacggaagctgaccgctgggcgcaggcagaagagcagaagtatgagatgctggagaatgagtactctcagagggtggctgaccggctgaaagcatcaggtctgagcggtgatgcggatgcggagagggaagccggtgcacaggtgatgcgtgagactgaacagcagatttaccgtcagctgactgacgaggtactggccctgcgattgtctgaaaacggctcaaatcatatcgcataacccactgataatgtttactaccggatgtagcttttttcatgcccgagatggtcagttagttccgcattaagcaccgtttcgacagttaatttcgtctgcatacgggaaaactgattgaggtcggcctcggttttgaggcctttagccagttcagccgcaagggctttaagcttcttttcgtccataatttacctgtctccgttgttggagtgaacatatcaaaaacaggcaattacacaatttaaattacagtctcaaaacaaaaccaactgtcaataatagtttaccagcaagtttactcgcagagtgctaggctgtgtcccttaattacgtaagcgttgataaaagagtcggatgcagcccagtttcaccatcgccaggtaatttctcgccgttttgtcgtaacgcgtggcgatgcggcgatattctttcagccgcccaaagcaccgttcaacgacattacgattgcagtacgcatcacgatgaaccacggcagcctgacggctgcaaattgaaaaccgcccctgttcgtcctgttgttgccgcacgctccgcagaatatgtatgctctgaggacctgttttattaaggactcatcctcttatgttgacccgcaaaagtatagataccgttctgctctctgttggtgctgagaagctctcacaacgtgaatgggactggatgaagatgctcaaacccatggacccaccacctgcgatggtcgcggcctccatcctggagcgccgtggcgacacagcagcccttacccggctacaggataccgggggttaatccgtcctcatgttgtccgcttttactccccgcccgctgaaacgtctgttcacggccaaccagtgctggacgtccttcctggatgcgggcggtctgcgcgatatcgaggttgaggctgtcacaaaaatgctggcctgcggcacgcggatactgggagtgaaggaatacaactgcgataaacctgagtgcccccacgtcagatacgtcactaactcatgcggcagccgtgcctgcccgtcctgcggaaaaaaggccacagacctgtggatagcgacacagctgaatcgtcttcctgactgcgactgggtacatctggtcttcaccctgccggacacgctgtggccggtgttcgaaagcaaccggtggctgctgaatgacgtgtgccgtctggcggtggagaatctgctgtatgccgcccggaaacgggggcaggaacccggtatcttctgcgccatccacacgtatggccgtcgtctcaactggcatccgcatgtacatgtgtctgtaacctgtggatgtctgaataagcatggtcagtggaaaaagctgagcttcctgaaagacgcgatgcgttcgcggtggatgtggaatatgcggcaacggcttctgaaagcgtggtcagaggggctggcgatgccggagtcgttgtcacatatcacgacggaatcacagtggagaagcctggtgctgaaagccggaggaaaatactggcatgtgtacatgtcgaaaaaaacggccggcgggaggaatacggcgcgctacctgggtcgttatctgaagaagccgccgatagcggcctcccggctggcacattacaacggaggggcgagcctgagcttccgttacctggaccacaaaacgggagaaacggcgacggaaacgctgacacagcgtgagctggtcgcgaggctgaaacagcacatcccggagaagttttttaagatggtgaggtacttcgggttccttgccaaccgggtgtgtggagagaagctgccgcaggtgtaccgtgctctggggatggataaaccggaaccagtggcgaaagtgtgctatgcacaaatggtgaaacagttcctgagtcgtgacccgttcgaatgcgtgctgtgtggcggccggatggtataccgccgggccattgcggggctgaatgtggaggggctgaagaaaaacgcgcgggatatcagtctgctgaggtatatgccggcctgacacaggctaagtgtgcccgggaatcggtgcaagtataaaaaacagcctgttgcggtataagaatcgaccatgcagagagagaaaacaccggagtggtggggaaaaacagaaaggctcacgtggcataaggcgcggacaggggtatcgcttagtttttcaatttcctatacgtcgagtccacagagaaaataacaattctgagttatctgcgggaaaggttatttatatgttactatctcattctctcactggcgagattataaacgttactacgttctcgttttcctaccgcaacaaccgcaataattaacatatcgtcaatcacctgataaaccagacgaaatcccgatgcacgtagtttaattttgtagcagtcctttagccctcttagttttgccgacggtatatgaggattttcgctgcatttttttagttttttcacaaactgttgttgaatggtcttatccagtttcagccattccttgagcgcatcatccctgaattttaccgtataagtcataaataactgtccaggtccacatcatgcagtggctgattgctgcgttcagtaaccagtttcaccagttcctggtcatccagagcatcaagcatcctttcatacagttcagcaggcacacagtagaatgccggctgattccggttaagaatggcgacagggtaaccatctccggcactgactgtagccataggattttttttcagttcactgacacttgcactggtctcagacagaatgatgttaggcataacgcacctttgagactgtttaacaagtcttaacgatatccctgaaacgaccacttaacaagtctttattactcacacttttgcaaatgcattaaatgctgcttgtattattctgatgccgcgccagcggcatgtaaggcggcacgctgcggttacatgtgataccggagtaaaaccgccgaagcccggcgtcagccggtactgattgacagatttcaccttacccatccccagccctgctaagccatacccgttttcagccatgacagagcttctgtgcgcggtcggagtggtcccgacgagggtttacccgaagtcggggcgtatctccgcgttagcgggccgtcagggccgcttacgagcgtgtactcgatacgtccagccagaagactgacagcgatgatgatgtagttacaacattcataattaaaagcgactctgttcccgcccttcgggcgggggcagcggcgcttttcagttatgaggaggggcgttgtggtttcagttctgcgtcggacttgggttttttaggggtttatgtttgcatgttgtaactaaatcggcttcggtcggggcccgccgcttgcggtgggaggtgcatatctgtctgtccacaggacaggcagtgaacaggttttctttttaaatggatgtaattaagtagtttaaaggagatataaacaggtgtttaaaagatacattgcaccctgtaaggctgacggctggcgctttatgacatgaacggttgtaaccttatggggaagtcccttgcagtttaatgtggataagcaaaattccccgtctgtgaggcgtgttttgtatcaaaaacaggggggaccggatgcaccagaaggtggatgatgaggttgttttttgtatgtagcgctgattttttgtgcactggcgggcttcaggcgtgcgaatgcctccggcgcgtgccggattattctgaggaggtcactttcagggggaagctgtggccagccggctgtaattacggttacgtgacagaatcatgcgctccttcacacgacgctccacttcacgttttaccgcctcacgactggcagtgaagcgcccttccgagatttcacgcgtcagttgccgtttcaccagggtgacaatatcctgacgctgcctgtccgcatcacgacgcgcacgggcacgcttcattccacgggacttaagctctgtctggtaactgcggaaacgctcacgcacaaaacgccaggctttcgccatcagttcatccatacccagggtatccagtccctgcttcttgcgctgtctgttttcccattccacacgactgcggcgcgcggacgccaccgcctcctctgatacatcgagggcggcaaacagcgccggtgtgaacgtgatgtcggtcggaatgttgcagccgataagcgggtcatattccgtctggtaggtaatcagccccagctctgccaggaacgtcagggcacgcgtggcacgggtgatggaaagtgttccggcatcagactccgtcgccagtccgcactcaatggccagcgtggtgatggagcactgaacgcggttggccagcgggtcatagtggaaacacagcccctgcagcagcgcatcaatggcccggcgacgtagtaccggtggcatgcgccgacgcagaccaagcgaacgggcatgtgccacatgaatggcgaaatcaaaacgggaggtgaagcccaccgccttttccatcagtttcccgcagaacggaagcgtccgggcaccttcacggggtgtgaacaccggattcgggttctttacctggcggtaataagtttgttgaagatcagtcacaccatcctgcacttacaatgcgcagaaggagcgagcacagaaagaagtcttgaacttttccgggcatataactatactccccgcatagcttgattgttggctatacggtttaagtgggccccggtaatctttctgactcgccaaagttaaagaagattatcggggtttttgcttttctggctcctgtacatccacatcagaaccagttccctgccaccttatggcgtggccagccataaaattccttaaacgatcagtaatctagcatgctacgccacaaagtaaagtctttactttagtatatccagtctctgcagttcatctttgatgattttctcaacgaactgagcctgtgttatcccctctctctcgcagtactcaaccatgagatctttcagaggatttttgacaaaaacttttatctctttgtgtgtaagacgttttcttgcaactgcggccatttgtttctcagagtcagtcataggcttacctctgcgcacaaaccgcttttgactcaatgaggaagtcactgcattttctgtctgcgacatctcgcctcctcaatactcaaacagggatcgtttcgcagaggatactacagttttttgaaatcagcgacttgagaattgtggcgaagatctgtgttggcgcacaaatcaacgaggattactgtcgtttaatgtgatttaaactgtgaaatagtatggttttcagttatggaaacgccgggagcgaggaaaacttgctttttcccgtttccggggttggacaactaagcaacgcgaaggcgtcagctacgatgttccggtgactgctgatcagcccctgttgtattcagccattctccggtcatctgcagcagttttctggcatcgtggcttctgagcaacggaataccagcctgcctcagaacctcttccagaagaatatcccggcgtctgcgttccggtcgtaaatggctggcatcatccagctctactgctgcaacaatactgaacgaacgcagctcaacgataaccacatcaacatgccactgagacaccatcctgaataactgccaccactggcgtgatcgtggccggatattcccgttcagctggacgatatccgcgacccgtacctgcggacacagataccaccgcttcggtgatgctgccaacttactgatttagtgtatgatggtgtttttgaggtgctccagtggcttctgtttctatcagctgtccctcctgttcagctactgacggggtggtgcgtaacggcaaaagcactgccggacatcagcgctatctctgctctcactgccgtaaaacatggcaaatgcagttcacttacaccgcttctcaacccggtacgcaccagaaaatcattgatatggccatgaatggcgttggatgccgggcaactgcacgcattatgggcgttggcctcaacacgattttacgtcacttaaaaaactcaggccgcagtcggtaacctcgcgcatacagccgggcagtgacgtcatcgtctgcgcggaaatggacgaacagtggggctatgtcggggctaaatcgcgccagcgctggctgttttacgcgtatgacagtctccggaagacggttgttgcgcacgtattcggtgaacgcactatggcgacgctggggcgtcttatgagcctgctgtcaccctttgacgtggtgatatggatgacggatggctggccgctgtatgaatcccgcctgaagggaaagctgcacgtaatcagcaagcgatatacgcagcgaattgagcggcataacctgaatctgaggcagcacctggcacggctgggacggaagtcgctgtcgttctcaaaatcggtggagctgcatgacaaagtcatcgggcattatctgaacataaaacactatcaataagttggagtcattacccaccgcttcatatccacaattctgaacagtcctttcataaaactgacttccctttctgtcagcagcgtcttctgacgttgccagtattcaccgctggacagtattcgctctgcatcaccggtccgtatacctgccgcatgcaattgcatcagcaaaggagaatgctgatttacacttctgaggataatccggataagtagcgctgcaataatcagaacaatgagaataaaaataatcaacttcattatttatctgctcctggcaattaattattgctgaattattaccttacatatttccatatctcttttggtatcctgtcatataactttccggtgattaattccgctaaccggtggtctgctgctgaattaaatgattcacgttcactgtttgataaactgtctctgtttttatgaataattttttccagcgtattgactgatgaacatcgccgtaactgatacagccattcctgttttgtttttgccatttttattcccgctgagaacagatgaatgataggattgccatcgctttatttacggtgcctccatatccaggggggaaccggatcagcatctgaccagagcccacgtttttgttgtctggcttcgttctgaaggacaggtagcacgggatctgtgttgtactgctcatatacccaggcagccccggcccggaccataaactgattaatattcattccgtctggtgcataaacctgcccgagaatacgaccataacgatctcgctgaaaatatgtgacggtaaccgtttttcctgcgaccagtgatttcatcatgtctgttgaccaccggccataatcctgtttcttttccggtgcatcaatatttaccagcctgatccgcaccgccttcagtgagtccatgacctcgatggtatcaccgtccagtacacgaacaacccgtccatgaatatctgcagacaataccggccatgaaaagataaataataccagtggaatatatttcctcatgttattctccatatcagataaatggcacctgtatttcagaccccctgaatagggcagcacacattgaataacaccttattttgtgaaattaatcacaaaatataaaaaatcgacttaatgcggaaaagactctgtgctactcagaactgtccggtcatactgcccgaccggatattaagaagcaaatctcataaactcctttcagccttctgactcctggagtaatgaaggctgcttttttcgttacacccttccccggcagcgccggtggcagtgttcagccctttttcgggcaaactgctgccagtctgtcctgggcgcaacacaggcagcccgtcgccatgcttctgccgcttccatccagcgtccactgacttccagaataacggcccgcccgccggcacgctggtaacgccagacctggccgttccgtgaactgtttctgaatccattcattttcttgtccccctttagttgtcagtattattgtgataaataattcagtggactggcttcctttcggaagcggaagttatttccttttaagccttatttccctgcctcctctctgatggaggcttttttttatttctcatcaagcacggcctgaagttctgcctttatccggttctggcggcggattttatccagacgcgctgccgcatacgcttcctcctcctgagaaatatgctccgtcacatacccttccgtgtcataccggcaggcaccggctttcatggcacacagatagctttctgaacgggtgatggccttcagcgccctgcgcagttttttatgcgagagcgggatattccgctgcgccacgtcttccagtaacacgtcccggataccgcaggccagaagccggagggtgtcaccgtcaaataatcccggccaccagggtttcagggtattcacggcctcatccagcgtgggcaggttcagataaatggacagtgcctgtctggcctgcgctttttttgccgccagctctgcttcccgggctgctttctcggccagcttctgctttttcaccttccactttggtggcgtggtgacattgatgatggttttccggctgcggacaggcgtctccccttccgttttccgcttcagtgtcagtaccggtcgcttctgctctgtcatgctgtgttctgcctctgacgaatcatcctgttacggtcaaagataccacacccgtcacagcgccagaacgcccagcacagccagatgacaggcgtagaataccgggaaaaaatccccgggccagaaacggggtaatgattttcctgcacacgacaccagaccaacggtcagcaccgtcatcacaagaccggccaccgccgctgcatcactggtggcaaggttaagcgccggaataacagccagcaggcaggcgaccagcgccagacgttccgctctgtcttccgcccggtacagtcggtggctgactgccagcatcagcagcccggcaatgccgtaactggtgccggacaaaggcccccaacagggccatcagaagaatggccgctgctgtacgccacccactgcgcgtttcacaccacgtcagcacctgtgccgccactgcaaaggcaaacaggatattcccctcataccagggaaagcctgccagataatatgcgaactgggcaataattccccatccccacagccggttaatggccggttgccggatatgcgcatgacgggacagattcagcccccacaccagggcaaaaagcggaaaggctcctcgcccggccagaaacatccattcctgcttcagctggaatatcagattgatgtggtccagtaccatcagcaccagtgccaccgttttgatgatatcccgctgtccgggtgaccagaccagaaatgactgcaaccaggtatcggtccgtgcagccagtgaatcgttacgtgtcgtgttcgtgttatccgttgtcatcagtgtttgtcttcctgtatcagtctccgcccggggttttctctttctgcaggtcacgaaccatatcccgctccatctgctgcagacgctcctgcagcagattttcccgggcaacctcccggaccgcttcccgctcccgttgtgattcacgcagcacactctccggcagcgcggcttcccgttcagttatggcagcccggtcacgctcctgcccggcaatttccctgacagccagctctgttttgccgtccggcaggtcaggtttgttctctgccattttacggacaatttcatctgcgcgacgttccgtttcacggcggatggcctcttcagcctgccgctgtgccagtacctctgccgtgaccggttcgccatttccggctcccggctggacgctgttgtccgggatatccccccacacgcggccaccggtgatggcaccgggattccacggacggccttcaccggcaatgcttaccaccacaccactgtcaggattatcacgggcaatccggacgccatcctgcatattatcagccagcaggctttctccgttacggctgccctgcagggccacgaactgcgcatccccgctgccttttacccgaccttcaccgctgaatccccgcagcccgtttccgtcatccgtggtcagcgggttcagccagataccggcggacttgccgttacggtcaaacgccggcagtgccacatacggttgcggatatttacgtcccggagcaataaaccgtgcaggactgtctcccccggccagccccgcctgacggagaacggcacggcctgccgccacgtcccgcagctcccgcgccgtactgaacagccgctctgcattcatgacctcccggtccggtttcggctcaaacacatcgtgggcagttcctttctgtacggcattgttgatggcatccgtccagccctgacggttatcggtgtacacctgcacatgctgcttcatacgcgacagggccacgtaggctgactcaaagccggccatcagtttccggttaccttccgtgccttcaagcgcgatggcaaaggtttcactcgccccctgtgcaccgtgggcggtgatggcataggccaggtcaatatgttgctctgcccgctcctgaccggggcgaatcacacgggtctgctgaccgtcagaaagtgtgacactgtcaccggaaaccgctgtcaccgtccagacgctgttggccacataaccgcgctcccggtcactcttcgtgaagcgtatcctgtcaccggtccccacccggatggtgtccggggtgtacagcgtgacgccttcagccaccgcctcccggggcgaaatcagccgcgtgttaccttccgcatcctgcagggttatcagcccgtcatccttgctgataccggcaatccggtgatacacattatccaccagggcaagggcatccgggttattctcccaggtggagagacgacgcagctccccgtcgcgtatattcgctgtgttcaggacaggcaccatgacctgcacctgccccagttctccggccttttcccgtgcatcatgaatcatgctgttcagtacgcgccgatcctcattcaggtgcgtgacaatcagcgtctgctcccgtgcttccggtgtcctgccggtatagtcgcggacaatggcttcatacagtgtcatggggacatccggaaaagcctcgcctttcagcatcgccttctgctgcgcttctgccagtttcgcttcctggctgtgactgaactccgtcacggagtgctccggtgcccatgcgccctccagacgtggcacctgagacggtttcacacgctcaagcccggacagcgccctttccacatcccggttaatcaggctgtataccgcctcccgcagttccggcgtctgacgcacaatctccttcatgatgaccacatcggcagcactgcgcgtctgctggagacggaaaggctgaccaggcgcgatggcctgcagctggtccgtgtcaccgctggccacagcccgaccgccaccgaccgcaatcagggcgtatgcccgtgccatgtcggtattgcccaccattgagctctcatcgagcaggaacagcgtgttgctgaaatccggcgtttctccgctgcgctgctgcagctgcgtgtcatgcagaaaggacgccagtgtctgcgcatccacgccggcgctgcgcatctccccgaccgcgcggtgcgtgggacccagcccaacgacacggggacgttcactctccggcagcatgttcacggctgacatcaccgcccggaactgtgtggtcttacccacaccggcatagccctgcaccaccgtgaaacggtcggacgtttccagtatcatacgggtggcggcgcgctgtcctgatgtcagtttctccatgagttcgccaggtactctctccatcagcggcgtgaccgcctccttaccttcgagaatatggcgaagaatgctcttttctgcctcatacgacgcacgggaaaccagcaggcctgtgccatagccttttgccacatccacatacagtaaatcaccccgttttatctgcgcattgatttcccctcccaggtcagcaaaaccggttccttctgcagcaaacgacttcgcctctgtcagcaggtccaccatgctgaaggccaggtttttactttccagcacaggaagggccagatgaatggcctgctgtgccggcgtgtgaagcccggctttctgcagactgatagcggtttccagcaatgtctcaccggcacgcgcctttatctgctcagaaaccaccgtaaaggagggatggcgggcaagtttttccgcagtacgggtttcatccagtgaggaatacagccggacatcacgaccactgcgggccagaccgttcagggtggcattgtccattgccatctgtgtgacggaggcaaaaaccgtcgcactgtcgctgacggaatgcccgggcgtttccacccagccgttctccagcttcagtgccgtgaacggtgaatcgctcacaggcagggtggccggttcagcccgtcccggcacaacaaccgtcatcgcatcttcactgacggatgccacctgcaggcggtcaccgccggagacgcggagtccgggaattttccctgtcaccctcagacgctcaccgtctgccaccggcattttttccggccggaacagcgaccagctgctgtccagggaagaaatacgcaccacctgcgtttcgccctgcgcattccgcagggtcaggctgtgactctgcgccgtcacacgttctgtcacatagcggtcatgactgcgtgtctccgggttccactgctccatcaccattcccgggcggtacatatcccgcagataacggctccggctgtccagccagaccggtgacagggcggtcatggttacctccgggtgtccgagcacgccctgtgttttcagctcactgcgaatggcctgtgtcagtatggcctgttcccgtactccgctgacctgtgccacgctctcttctccggctttcacgctggccgcaaaatctccggccagccgggcatagcggacattacggtccggttcactgatgatggtggccggtcgctgttctcccccctgccagcgatatgtgttcaccccggcatccttcatggccatcagtgcactgcctgtaccggttcgctgcccgctgtcggttatcaggacctgtacgttatgacgtgcggcaccatccagcagggttaacgtctctttcagggagagtttttcgccctggtcgacgataacggtactgcccggcgtgaaggccatgccttccagcaactgacgacgtccggttatcagttcaccggacagccgttcatcctgcttcaggttcatctgcgagcgacggtcagcagcgataatctgcacctcccgcccctgctcccgggccatcatgaccagttcagccacccgctcacgctgcccggctgcaccgccctgcccggacacaatggccagcgacggacggtcctgtgccagaacactgacggcatcgctgtaaccggccgtccgggggacacttttctccggatgtacggtcacccggttctgtttcatgatgtcacgactgagtgcccggactgacagctcatcgagcatatgaatcccggacgtgaacagccccttctcacgatcgagggggataagctgctcacggctgatggcctcatcgataccggcgcgtgcccgttcaatcacaccattttccggcggcagtatgccgaccgtcctggccagcaggtccgtatacataaactgcactttgcgttcacttaatccggcaatggcctgtgtcaccgcctgctgcacatccggcccgtcctgtgaagcgggtccgggtgtctgcgtgcgggtatatgcccgttgttccgctgcgtcacgatatgcccggatgtcgaacccggtttccttcagcgtctgcatccactcggccattctgacttcaggatcgacgtgctgtttggatttacgcgtatccagcgccgccacatcccgggatttcagcgaggcgtcttccccgacggcctcccggatagtctgtgagcgtccggaaaaggcctccaccggtacacccggcatttcccacataccgtgcttaccgaccacttcagtttcatagcccagcgcctcaacctgctctttccgtttttcccggtagagcctgccaaaggcaatctgattagcgtacacattctcaatgaaccccgttttccccactttgtcactgctcagtgtcttccattcgccgttatgctgcgtaacattagtcaccaccgcatgcgtgtgtaactgtggttcctgatcgcgactggtgtcgtggttaaacagtgccatcaccagattaccggtcagcaccgtttctgactgtccgtccgtcatcacccgtgtggaggccagtgcctccacctgacggacagcaaaatccacggcctggttatgtgcttcaataagacgtttgtcccccccgagcatggccatcatggagacacttttgggagcggagaaggtcaggtcgtagccgggacgatgcctgttactgccatcctgcatgcggcttaaatccgccccgtccggcagcctgccctccagaaggcgggtaaagacatccttatcgacactgccctgcagccccagttgttcagccccctggccggcccagcgttctcccatgctgcccagcacatagtaattatccttgtcggtataataattatcggcacttccggccgatctgacctgcgcgatactcatcataaacgtatatccctttgacatatcccggtatcaatcccacaatagatatacacaagacatatccacataaggaggcaaataatggaaaccaccgtatttctcagcaaccgcagccaggcggtcagactgccaaaagcggttgcattgccggaaaacgtaaaacgcgttgaagtgattgctgtcggacgaaccagaatcattacgccagccggagagacatgggacgaatggttcgacggacacagcgtcagcgccgattttatggataacagggaacagcccggcatgcaggaaagggagtcattctgatgctgaagtttatgctcgataccaacatctgcatttttacgataaagaacaaacccgccagcgtcagggagcgttttaacctgaaccaggggagaatgtgtatcagttcggtcaccctgatggagctgatatatggtgcagaaaaaagccagatgcctgaacgtaatctcgctgtgatcgagggatttgtttcccgcattgatgttctggattacgacgctgctgcagccacacacaccggccagataagagcagaacttgcccgtcagggacgccctgtcgggccatttgatcaaatgatcgcaggtcatgcccgcagtcgggggctgattattgtgactaataacacccgggaatttgaacgtgtgggcggcctgagaactgaagactggagctgacctgttcatcagaaatcatctcccggctcaacatcctccccgcgttcccggtgcacattgatgttcacctcttcacgacgctgcatctgctgccaggtgtccggatgattttcctgctgccatgcctcataagcggccatatccaccacttcaccggattcactgatcccgggtggcagttgctgttccatctcttcttccggtttcattttcagctcctgctcaatcccccctgccggaacattcacacctgaatctgacttcttatcgttgatgacaggagacaccggctgttgcggctgttgcggctgttgcggctgttgcggctgttgcggctgttgcggctgttgcggctgttgcggctgttgcggctgttcagcctgagtcacgtcttctccggaaacaacctccgggacatccggttcgaagaggctggccatctgacgaccttctgcttcccttgcggcaagtacggcactcagacggttctccatttccgggttgatgtcacgcggaataaactccggagcgaccttcggacgttcctgatatttcagtgagagtttgactgccggatacggtccgggcagggtgacatagcaggtcagatccggcagggactgaatgtcggaataactgaccagcgtctggcgctccatatccttaccggtcgataccccgtcacgtaccggatcagcaccgtaggaatactgttcgctggctttcaggtgctctttctcaccaatttcaccgacagcgaactccgcaatcttatggctgggagaacggaaaaaggcacgggtgttcatgacgtcaaacagcgtggcagccgctttctcaccgtagatatcttccagctgggcataggactggataccaaacacataacagccaccgaacttacgggcttccggcaggatctccaccaggtccggcagtttgtgtaatgtgggtaactcatcacagaagaaccacacacgacgattacggttttctcccattgccagcagaccacgaatggcaatggacagccacatggagatcaccggcttcagggaggcatgagtgtcggcattcgacgagataaacagccagccgtttttctgatcctcccggacaccacgcatccagtcacggatggtgaagggctcaccgttatgctcaatcccctgcaggtaacggatggctttcacatagttggtcagcacagcacggatggaaatcgccgttttctcaattttctcttccaccaggttggccgccggtgaattacgcaggtaggtacgcagtttttcaattttgatggaaagcagtgtgtccaccagtttgctgtagctgcggttggggtcattacgcatcagatacgccgcttccgcaaaaatggtacgtcctgaaccctgccagaacgggtcctcttttgtgcccatcgggatcagagtatttgcggtattatcaaaatccggctgtgtcaggcactccttccacaaatcccaggcggcacagcgtgcatccagcggattcaggatcttatcgatggagggatcatagtaacttttaacaaattcccctgaacggtcataaatcaccaccatatcaccacgctgacgggcgtagttggccagacgacggataacctccgacttaccggccccgaccgtgccgtgcagacagaagttctggatttcggaatcccggataatcggcagatcgccaatccggatatcggagtccctgccgtcttttttcagcatccgggcaacgtctttcggattgtctgtcagttgacgaccacctgtgacttcattttcgctctgttgtttaccctgacgccccagaatccaggagacaacaaagaaggtaatcaggcaaataaccagtgccacaactgtggccagaacgaatgcagaccataactgctcaccgcaccagatcatatatttatcatgcagtacctgagcagcgttcatccggaaggttttgccgtaatactgaatctcatataccggctgagacttgattaaatcccgcatgccttccagcgtggtacaccaccagtaaatacagccattcacaaacgtctgccagcttatttttacccataaaaccagaccaacgagtatccagaaaaaaataaacaggcagtaaagcataatattggcgatctggctgaacatgcggatacgcggtaatggctccaacttattgatagtgttttatgttcagataatgcccgatgactttgtcatgcagctccaccgattttgagaacgacagcgacttccgtcccagccgtgccaggtgctgcctcagattcaggttatgccgctcaatgaggtgtactggcaatagcggacactaccatttgttctttttttaagcagccatctgatgatatttttccctgaaggctgccggggagatattccccagacgagagtgacgacgctgacgattgtagaaaatctcaatgtattcccgtattactgagatggcttcatcccggttattaaaacgatagtggctcaggctctcatttttcagcgttccccagaagctttccatcggagcgttgtcgtaacagttacctttacgcgacattgatgttttcagaccaaactgctcctgtatgacccggtaatcgtatgcgcagtactgtgaacctcgatcagagtggtggattagcccggcaggtgggcgctggctcctgagcgccataaacagggctttacctgtcagctcttttgtcatgcgctctcccatggcgtagccgacaatttcgcacgtataaacatctttgatgccagcgaggtacaaccatccctcctgtgtggcaacatacgtcaggtccgccacccagacctgatttggtgctgtaggagcgaacgtctggttcagcagatttggcgcaactggcagattgtggttcgagttcgtagtcgctctgaacttgcgtttctgcttacagcgtagccttagctccttacgaagacgtgccagtcggtcacgaccaacgatgatgccattctctgccagctccgtctggagccgccgggttccatatgtttcgcgagtgcggatatgtgccaccttaatctccagttttagccgctcatcactttgttttctgtctgagggttcatgctgtacccagttgtaataaccgctcctggatacaccaaatacctgacacatcgcttcaatgggaaattgttgtcgccattgttcgattaacgcgtatttttcagcgactcctgtgcaaaatacgctgttgctttttttaatatatctcgctcaaggcgagcttcatttaacgccttacgcagttgcagaatttcagattccagttcagccaccgtgcgggaaccaggagtaccgagcccttttctggcggcggtaacccattgtcctaaagtgccttcaggaagagataatcgggaagcgccttcactgatcgaaagttgattttcaggaaccgttctgacagcttcggctttgaactctttagagtaacgttgggtttttctgctcattattagctccttctgatgccattctatttcaggaaggagtgtccgttaaactcaggctacctcagggtcagtcctgcatccccgctgtcgtggtacagccacaattggcggttaatcacaagagaagtcagaatgatgcctgcggcatcagcactcacggtacggtcaaaccagtttgcaccgttcaccatgtggaaacggtcgccatccggcatcatgtaaccgccaccgtccggcaggcggatgaagtgccagaagccgccctgatattccggcatcattttttgcgccagcgcgtacacatacgcctcggcatacagaaaatcgttgccaaacaggtaaggcagaaaggagagacggtcgcactcttcaacattcagagtgacaggtttcgcatattgcatgatgttttcctccgttgttcacatattccgcgacaccgggcggtgccgtgcctccatcttagccgggcgataaggcggggatgcacagggcgcaagggaagccgcagcctgacgcacacggctggcgggagttggagggtgaaaccgcgaaatctgagcgggtgaacgaccggaaaatcacggcagacgggaaggagggaccgggtgaccgcagcggcgaagcgtacccttgcatcaacgccagcccggctaagatggaggcacggcacagcccgcacacgacgctgaggctacagagctgaaaacccacaaggccatgccccgcatggccgggtaaagcggagccggcggcgacttcctttgtgctctgtccggggaccggcaggaaaaaggcggggatttaccccataagcgctaacttaagggttgaaccatctgaagaacgcgacgcctcggtgcctcgttaagacgatgcctcgcgttcttcaattgcgttttgtaggctgtcagggatactgtcccacgaatggccacctgtaagctccagatgaccatttttgttattctccacaacgcgttagttcttcttttcggatccggcacttctggggggaaaatccagcgatggctggattatgtcgtcaattaaaaatgcggcgagtagattagcaaatatccacgctttcgcgagttcaagttcctttgcacgcaaagcatccaagtgcagcaaacttttgagccgcttaaaagccagttcaatttgccatcgcagacggtaacaatcagccacttgctctgctgaatattcatcttccgataatgatgttagcaatagcacatggcccgctgcctccagcgtttccgcctgaactactcgtccttttcgacgattctcgctgagcagtcgggttttactgattaatgctttttcgggaggaagtgatacggcaatgagacgtgcaggaaagggagctccagcttttttattacctgaattgcctatcattacagtggtttcaccgttcttaccgcaatccagcccgcgcagaaaatccatcatgtcaaagcgcattccttctgcagttaaccagcgcaatcctcgccagtaaacccagacgatataatcagcttctccaaaagcaagtgagcggatacattcgggacgcgaaccgaatccccggtcagcaattcgtatctcgtctgccgtttgcgcaaatcgatccagccgttcagcgtctctgctgtcggttagctcaaaatcagtgaactgacaggtatgaggatcatatcccatatgtagtcgccattcagcgctgccgcccccgggcgcgctgattgctgttccatcgacaagacgcaatcaaggtgcggcgggagtcgatggcatgcgcgtaagtgaactgccggactacctgaggcaccactggccagagctgaaagcgcaactgctgtccggcagataccgcccatcccctgtgagaagagtatccatactgaagcccggcggcggcgaacgtctgctgggcatccagatggtggtggatcgcttcatccagcaggctatgatgcaggtattgcaggcactgtgagatagctcgttcagcgacaacagctacgggttccgtcccggtcgttcggcccaccaggcagtgatacaggcccaggaatatatcggggccgggtatcactgggtggtcgacctcgatctggagaagttcttcgaccggataaatcacgacgtgctgatgagccggatagagaaacgagtatcggataaacgggtgttgtcacttatccgcaggttcctgaatgcaggggtaatggacgcaggtctggtaaggccggtgacagaaggaacgccgcagggcggcgtgatctcgcccctgctctcaaatctgttcctgcactatgcctttgatatgtggatgcaacgtcagtgtcccgacgttcccttcgaaaggtacgccgatgatggtgtgccacgaaggcgcgcaccagggacgtgagtccctgtgcagccatgctgcacaagagatgagggaaggcccctcggtgtcggcgtgcaggcgcaggcaccaaaccacccactgctgctgcatttaagagatgtggtggtgagcgagatgggaaaaggtcggacgtgagccgatcaggtatggatcgaagagacgaacgaaagtgaaccgctgatgatgtgtcgaaagcatatagatgatgtcaaaaccatggttatccggctccggcgttaacgtcacgggaagcagtgccggcatattttttccggaaggcagcaggccacctttccggtattgatggcgccagttgaagagcaggttatcgttgattccattttcacgggcaatctgcgccacacaggctccgggctgcagtgactgctccactaaggcgattttgaactcataagggaagttggggcgccggggacgcttttttaccccggggacttcgggcataacggtactttcagtaagtacgacaggtactgtggaggcactcccgtaaagacgggcgtcaagttcccgctccgacatacctgcgggcagaggccatgaaaggccagctttgcggaagcgcacaaacatactacaaactgttgattttggtacgcccaggcgacgtccggctacaacgaggggtaaatgttcttcaaagtgaaggcgtaaagtgtggatggtgcagaagatgccgatttccagcccccgttttcgggctgcatacagcagattctccaccgccagacgacacacgtcattgcagccgtcaagctgccgtggttccgtgccgttctaaatagctgcgcggaatagtagatcactttgagggaacttagcccggattgtgcgatctgatcaatcgccaaatcaaaacaaatcaccaaccggactgagcaatgccgatcatagcaccaatttcccgtgacgaacgacgcctgatgcagaaagccatccataaaacacacgataaaaattatgcccgcagactgactgccatgctgatgctgcaccggggcgaccgtgtcagcgacgttgccagaacgctctgctgcgcccgctcctctgttggacgctggattaactggttcacgcagtcgggtgttgagggactgaaatcattacctgccgggcgtgcccgtcgctggccgtttgagcatatctgcacactgttacgtgagctggtaaaacattctcccggcgactttggctaccagcgttcacgctggagtacagaactgctggcaataaaaatcaatgagataaccggttgccagttaaatgccggaaccgttcgccgctggttgccgtctgcggggattgtgtggcgaagggctgcgccaactctgcgtatccgtgacccgcataaagatgaaaagatggcagcaatccataaagcactggacgaatgcagcgcagaacatcaggtcttttatgaagatgaagtggatatccatcttaatcccaaaatcggtgcggactggcaactgcgcggacagcaaaaacgggtggtcacgccgggacagaatgaaaaatattatctggccggagcgctgcacagcgggacaggtaaagtcagctgtgtgggcggcaacagcaaaagttcggcgctgttcatcagcctgctgaagcggcttaaagcgacataccgtcgggcgaaaaccatcacgctgatcgtggacaactacattatccacaaaagccgggaaacacagagctggctgaaggagaacccgaagttcagggtcatttatcagccggtttactcgccatgggtgaatcatgttgaacggctatggcaggcacttcacgacacaataacgcgtaatcatcagtgcagctcaatgtggcaactgttgaaaaaagttcgccattttatggaaaccgtcagcccattccccggaggcaaacatggactggcaaaagtgtagcggtattaagcgcagctatttaggcataaattaaatttaacgaactgcatctacacagtcctccgtgccaaacgggcaacacacggaagggggatgtgtttcgattggtaacctgtaagaaacaactagtggaaatacctgtactttataatttcaagggtacgggtccgcttcgtttgatttgcgcgtttttaacaattttaaaacccagatatactgttccggatgtgaccccaccaaaatttccactgttttgttcatttgtcgggcaatcattttattgtccgcgctggcaatgcatgcatccattggtggccgaacttcaatagtcaggaaatgttttttttcatcataaaccgggaaaagcggtataatcatagcctgactgatgttcatcagacgtccaataattggtaatgtcgctttataggtcgcaaaaaaatcagcaaattcactgtattcaggaccatgatcctgatctggaaggtaatacccccagtatccctggcgtactgactgaataaatggttttatcccatcctcccgggaatgtaagcgccccccgaatttacgccgtacagaattccagacataatcaatcactggatttcgttgctgatgaaacatggcggccattttttccccctgagctgccagcaacattgcaggaatatccacgctccagccatggggaaccagaaaaatcacatttctgttattgtgccggatttcctcaagaatttcgaggcctttccagaacactcgtttctggaacttttccggaccacgaattgccagttctgccatcatcactatggattgcaatgctgtggcaaacatattgtcgacaattatttcccgttctgtatcactcttttccgggaaacaaagcgataaattaatcgtcgccctgcgacgagcttttctgctcaatctgcccacccaacgccctattttggccaagacaggatctctgaatgaagggggaaacattgaaatacctgcaatcattcccaaaacgaaccatgtaaaccagtaaacaggggaaagataatttttcttaaattcaggaataaactcggatttgtattttttcattatttagtgcgaatgcccctaccaaaatcatcattaatattttattcagagaatacatcaaaccttaatttaagtcctgatgttctgagtttacaggatatgcatccaaagagcggatagcagtttcatggatataatccaacaaagcgtatcgttttcgatactcagagcgttttttactagctatattgcttagcggctttcgaaccacctgtgttggtaatttgtaccagtcaccacaggttttccctgctacagactcccagaaatcactgtagacagcaacaaaggttttgcgtttctgataccagtatctcaattgccggaaaacatgactgtgctcactgactgccagacactcagagacattacaagctttcatcaacgcacaaaatgcttcaaaaataatccgttttgggaaaagcccataacaggcccgggttgcgcaccgaataacatcaggtccggtatttttgggggctccctgtaatccgccaataaaagcggtgtgtccattttgagtcagaatgactgaaaatgttaaccgggcaatcactattttattgaaaaacaatgacaacatcaactctccttcccggtcaaaaccactgggagaacactggatatcaaaatcctctccatctttcccgatgaattggactaatgataccgatttttgtggaaacaaaaattcagaatatttttctggcaaattttgcacaaagcgataatgctcaagaatatattgcccccgggaccaggctcgcccccctttatgcaaataaggtcgatgaattcttgctggcaacgtgggttgtattgccagaatatcctcaatgcactgcaaggaacgcagttcgtaataatatcgaatactggcaaccggatttatagcacatcgcaaaatgtacatgaaccggaattttctgtgttcccacttccccgggagtgaatctgaggaaaaaacaatccttttaaggaaaccgataaatgataagttacttacagaaaacatattgttacttgtgcttcgctaatgtgagtatttttaacgcagttttattaatatcaaaccgctcttctatatcctgttttgcctggcgagccatctgataaaaacgcccagggtcattaaaaaaatcattcagggcgcacatccatgcgtgcttattacctgctggtaacaatgtcccggtctggttgttctgaataacatcagggattccaccaatctgactggccagtacaggcacagaaaatgccgatgcttctgccagtaccataccaaacgattcgttttctgaaggcagaaccaccagactggcaacccgatatacgggggcggcaggaaaaacattgtcagcaataaaaacatcatcatgcatgcccatactgtcaatctgatactgtaaatgctcccgcagttcaggcgaaccagaccccacaatcaaccaacagaactgtcgtccattcatttttaaatggaacagtaaattcaacataaattcgtgccctttttccgggcgcaacattcctacctgagcaataacggggcatccccttcgggacgccagccaagaaagaacatttggaggcaaactgtttcgggaatcaacgtataatttctggaaatcaaagcctggaggcacaacagtaacccgggttcgacacccttcctgctccagatgtgtcttcatacttgttccgggaacaatcacctcatcgcaaaaatgatttattgaaaaaacttttgtttttcgtgtcaaatatgtcttttgcctgataattctgaatggatgtttccaagtgaataaccgtactaaaccaacaatattgctatcatgcccactgtgacaaacgattgcattgggctgaaaaccatgaactattccgagtaatctccatgcagtagggatgtgaagactgtttcgaaataacgcgaatgtgatatcaattcccaatttactggcttcaaaagcaattttgctgttttccctgcagacaagcagaactgaatgccccattttcttcagggccttcatttgagcaacagcctgtaattcctgaccacctatattcggtgatgattccgtaaatagtatattcattattaatcatccggtaatcttggccccttatgaaaggccagcaacgatgaaaaaaacggtgttgtataataaaaaagtcgtcttttcagccatgcgcaactttcccgttctttggtgcttattcttcctatccgtgtagttcctttagcaggagcattcattcttctttctgttgtatataaataatagaatccaagttcttcagcgacctgaatataatccttgttataataaccttccggccaacacaaatgtttactgcattttcctgtcatttctttcaaatattctctcccgctcaacaagtcctgccgaagatgtttacactgttcttctcgtgaactgaactttttatcccagcgtgtatgagtatgcgtatgaacatgaaattcgaccagaccagattgcaacatttcgtttacttcagaccagcgaagcataacattatcagcattccctgtcgctatttgatgttcgcaatcgcgatgagaatattcctttccaggagagtgacgaaccggaccattcccaataaaaccggtaattaaaaaaatatgtgctttgagattaaactcttttaataaaggatatacctgaaaccagttgtcaaggtatccatcatcaaatgtcaacataacgcttttccgggggagttttccgccccgataaaaaaactccagttcatctgatgacagtgttttccagttgttttcagccaaccatttcatttgcttccggaatgtcacgggagaaagtgtaaccagtccggggcaatgactgacatgatgatacattaataccggaacatgattggctttgaacaaaatgcccccttcgtttaacatttaaagtttctggcacctagtaagcagagacaaaaggaaaacccaataaagctggtgcattttatttccagatactggatgaataacatagcaaacccatggcatatttgatataatttactaaattaaacatcaacaattcataaacaatgatatattatcatatcttttaaaaacgactcactaagaaccttgaggtggcacactgaatttggctacctgaacagtgaggtgtactggcaatagcggacactaccatttgttctttttttaagcagccatctgatgatatttttccctgaaggctgccggggagatattccccagacgagagtgacgacgctgacgattgtagaaaatctcaatgtattcccgtattactgagatggcttcatcccggttattaaaacgatagtggctcaggctctcatttttcagcgttccccagaagctttccatcggagcgttgtcgtaacagttacctttacgcgacattgatgttttcagaccaaactgctcctgtatgacccggtaatcgtatgcgcagtactgtgaacctcgatcagagtggtggattagcccggcaggtgggcgctggctcctgagcgccataaacagggctttacctgtcagctcttttgtcatgcgctctcccatggcgtagccgacaatttcgcacgtataaacatctttgatgccagcgaggtacaaccatccctcctgtgtggcaacatacgtcaggtccgccacccagacctgatttggtgctgtaggagcgaacgtctggttcagcagatttggcgcaactggcagattgtggttcgagttcgtagtcgctctgaacttgcgtttctgcttacagcgtagccttagctccttacgaagacgtgccagtcggtcacgaccaacgatgatgccattctctgccagctccgtctggagccgccgggttccatatgtttcgcgagtgcggatatgtgccaccttaatctccagttttagccgctcatcactttgttttctgtctgagggttcatgctgtacccagttgtaataaccgctcctggatacaccaaatacctgacacatcgcttcaatgggaaattgttgtcgccattgttcgattaacgcgtatttttcagcgactcctgtgcaaaatacgctgttgctttttttaatatatctcgctcaaggcgagcttcatttaacgccttacgcagttgcagaatttcagattccagttcagccaccgtgcgggaaccaggagtaccgagcccttttctggcggcggtaacccattgtcctaaagtgccttcaggaagagataatcgggaagcgccttcactgatcgaaagttgattttcaagaaccgttctgacagcttcggctttgaactctttagagtaacgttgggtttttctgctcattattagctccttctgatgccattctatttcaggaaggagtgtccgttaaactcaggctacctcacagaggtgatatgttcacctcagaacaacacaggtgctccaatgaaaaaaaggaatttcagcgcagagtttatttacggggtgacgctttcattaacctgtacacgaacttctattccggcatcatgacaggcctgcagctactgcgccatttccagcggatcgccctcccggcgtaccaccccaccttctttattccataactgcagagcatttttctgattgttccatctgatttcggggggtattccgggcaaggttgttcagtaaggcattccttcgtaaaatcagcatgttttttgcacgacggaaaccggcagccatgaaaatctgtctttatcatactctgaatcctgacacgatcccgggttataaaaaatttgcacaggctattgccaccgataactttgttcaggctgatgttcgtaaaattgacaccaatctttaccgcgcccggctcagtatccgggaccgattactgttttcgctgtatcgctaccacggtgaaactatctgccttgttctggaatatattcgcaatcatgcctataacacttcacgttttcttcgcagaaacgtggtgattgatgaagggcgcttacaacaacaaccagtgccggatcctgttgatatcgcgactgaagcgctgacgtatatcaatccgtctcatgggcgtttccaccgcctggataaaatgctgtcctttgatgatgatcagcaggcactgtacgaacacccgctaccgctggtgattgttggctcggcgggaagtggtaagacggcgctggtactggaaaaaatgaagcaggctgctggtgatatactgtatctcagcctttcctcatttctggtggaaaaagcgcgtactctctatgacgcttcaggggaagggagtgaggtacagaatattgactttctttcgctgacggaatttctggaaactctgcgcattccggaaggcagggaggtgactttcagtgcgttcagcgactggctgccccgcaacagggctatcgccgcactgggggctgcccataccctgtatgaggagttcaggggcgtgatcggtgcggttgcctccgggaatgggccgctgtcccgggaggcgtatctgtcactggggatacggcagtcgctctatggcatggaggaccgcccgacggtctatgtcctgtttgaacgttatatcgcatggctgaaacagtcgcatcagtatgacagtaatctgctcagtcatcagtatctttcgttggcaactccccgttatgatgtcatttttgttgatgaagttcaggatatgacgcctgttcaactgcaactggtactgaaaacgctacgtcatcccggtcagtttttactctgtggtgatgccaatcagatagttcatccgagctttttctcatggagttcgctcaaaagtctctttttccggcagcaacagggaaacgacacaaccgttaatatcctgcaggcgaattaccgtaacgggcaccacgtcactgcactggctaaccggttgctgagactcaaacaggtccgctttagtgctattgatcgggaaagtcaccattttgtcagaagctgcggtcaggcagaagggaccatccggttgctggacgacagggaagagacaaagcaggaactgaatgccaaaacctcactttcgaatcgtgtagcggtcattgtgatgcatccggaacagaaagcacaggcgcgatgctggttcagtacgccgctggtcttttcggttcaggaggttaaggggctggaatacgaaacggtgatcctttacaatatagtaagcgctgcccggcaggcatttgatgatatttgcgagggtcttacaccggctgatcttgagggcgaggcccgttattcccgtccgcgggacaggcaggatcgttcagcggaaatctataaattttttaccaacgcgctgtatgttgcgctcacccgtgctacacacaatgtgtatcttgttgaacagcaggtggagcatcctctgtggagtctgcttgcgctcacgcatcaggaagagcctctgaatctgcaggaagagatttccagccgtgatgagtggcagaaaaccgcacatctgcttgaaaaacaggggaagcaggagcaggcggacaccatccgtagccggatcctgcagacatctgagatgccatggcagattatcaccgcagaggatgcacgacagtggaaacagcatatcctcgcaggtacagccgataaaaccatacaactgcaggcgctggagtacagtctgatttattccctgttcccgctgtataatgcactgtaccgggaagattttaagcccacaaggcagcccagaacgaagactctgcagttactggagctgaagtatttccggccatactcaatgaataacccggttgccgtgttgcgggatattgaacgttatggtgttgatcaccgttcgccgtttaatcttactccgctgatgtcagcagcccgggcaggtaatattgcactggttcagttactgctggagcgtggtgcagatcccttgctgaccggtaatgacgggctggcggcttatcatcaagttctgtcagcggcggtcagtacgccccgttatgcgcaacagaaaagtgcgcagttgtataccctgctgaaacctgaaagtctgtcactgcaggtggaaggacgcctcatcaaactggacaaccggcagatggcaatgtttctggtgatcctgatgcaggcgctgttccacacgcatctgggaagtgctctgtttttttcggaagccttcagtgcggcacgactggcagagtgtgtggttcatcttcctgaagcgttgctgccggaacgccgcaaacgaaggagttatatcagcagccagctctctcaacatgaagttaacagcaaaaatccttatggaaaaaagctttttctgaggctgaaccacggacagtatattcttaacccggggcttaaaataaggcagggtgatgtgtggcgcgcggtttatgagttgcagtcaccggaagatctggggcatgatttgcagacttatctgcaggatatgtcaccggaattagtggatatgctgggagggaagaagggtttttacgaacgcagtgaaaagtctgtcggatactgggtgggggggatccgtcgtgccgctcaaaaagcgtgaggttccgtgctgacaggatgtaaccgaacttcatctgtacgtaagtacgtgatgtctcccgcccacttctggttcggaccactggcgtaaaaatcctgctccagcagattttctgacacgggcaggccatgtgcgcggtaactgaccgggctgaacttccgggaggcctttgccctcagcccctgacggcgcaggcttgccgccacggtttttacgttaaagtggtaaccctgagcacgcagttcatccgtcaggcgtggtgcaccgcaacgctgttttgaccgggtaaagaacgctgtcgcagtgctgacggaactgttgacgagggcttatccctgtccgccgctgacaccacgttcaacagtttgtggactgttcgctcgcttccggaaatcagggctttcatggcctttgcctgcaggtatgtcggagcaggaacttgatgcccttctttacggaagtgcctccacagtacctgtcgtacttactgaaagcaccgttatgcccaaactcccggtggtaaaaaaacgtccccggcggccttaatgccagtcagttaagcaactgactggctctttttcggggctgtggggtatttccagggcctctcctttaccactctcgggaaggccctttcccttctcgtcggtaatttcacaagttgtcccatacttgcaagatcgcgcatcagctccggtatacgtcccggtgaagcgccctgcaatgtcatcagcattctcatcaccattccgcatgattctgagaaactcagttgattcggccagtaacctttcagatgttccgccattttaatcatctgatatctcaccagattataagccagtaagacaccccacagctcttgctccacaagctccggctttttacttctcagcgtcagcctgctcagttgcatcgtctgttttatctccctgtatcccagttcgatttcccagcgatgactgtacagatccgccatttctcctccggggaagcgcatggcgtccgtcatcgacgtcagcagatggcagacttttcctttgcgcgtcacggtcagcaggcgggctgtcacttcatttcccagtcccggccacttttttcgtgcctgcgggctggttttcagcttcaccagatgatcgcctttacccagttttctgagctcttcatattgcgctccctttctgagaggtatcatccagtggcggtgttctcccgccaggctccaggcatttaacagtcccagtgagtaataacctttatccattaacgtcagagtgttatcgccggtttgttctataagttgctcagcaagctcattttcgctgttcttcatcgtgccgaaggctgcagccgtcagcagatggctggtcagttccatctggcagaccattttgacctgcgggtagagcgccgggttcccggcatgtgtctggcgggggaaggctgcatcgttctctggtgtatccggtgtgcgccagaacacaccatcgatggccagcagggtcaggccgcaccagtgcggatgcggcgtggcgttatgccagagctgcgctgttttcgtgaacacgcggcggacagcctcacttcccaggcgctggcgggcctgaataacggcactgggggcaacgaaggggcgattgcccggcagcatgatgtccaggcgattcacaatctggtgaagaggttctttacgctcaagcgccatgccaacaatacaccagaccatcatttcgaggggaagacggcgcttgcgtagcgttacagtacctgattcggcaaggcaacgagagatgagttcggggtcgaggtaatcccccagagaagtcagtgggttacgcagagaatcgtaacgggataccagatcaagagcctgtccaatgtgcataaaaaaatccggaaacaagtgagcgtttccggattcttacacagccactggatcggtcaactgatccttaactgatcggcattaggccgccggggacgtttttttaccaccgggagtttgggcataacggtgctttcagtaagtacgacaggtactgtggaggcacttccgtaaagaagggcatcaagttcctgctccgacatacctgcgggcagaggccatgaaaggccagcgttgcggaagcgcacgaacataccacagacagttgattttggtacgcccaggcgacgtccggccacaacccgtggtaaatgttcttcgaaatgaaggcgtaaagcttcagtgattcaggtccggtattccatacgatagtgtccacttcaaaatgatggacattatttttgtggaaccggaggtaacaaaccagacggtttaaatgagccggttacgtttcaggtgtctctggctggaaatccgtgcgtgattcgccttgctgcacaggtatgcatgtggttaaaatttatcatacgcgatcgcggcggtttttcgggtggtttgttgctatttttacctgtctgctgccgtgatcgcactgaacgcattctggcggtgcatacaattaagatattacgttaatatcgcgagggaatggtccccatattctcatagaagagcctctggcttatagcatcaacaaatacggctataaatatttcctgtttaataacgaataaaaatcatttgtacgcatttgtaatcgtcggtcgataatctctttatcttttttggttgcagcgtaatattcagcataaatattgcgaaaatctattgggaataaactttcactttctttatcatacagcatattacctgtattaagatcgtaatggattatactcaactcattcaatttacatatcaactgtagataaaggctctctatattatcagggatatcagctgtatcaatgtcactaaggggagtcccaggcacgcgcagcattcggaggtacacatctccgccatactgtataacaacggatgcttcatcgccataaaaagcattaaaaagctcagattcttgccaagccatttccagaatctcattgtactggttgccaataagatcatactttttatacaaagcagatgaatcattcacatcttcaaagatttctgctgtcgacccctgacctattaacttaccgagaatgggctccgttactatgccagcatgagaattattattctcaaatggaaaaggtgtttgaataatggtagatgttattttcatgtcatacctttgttacgatatttaaaaatcacaagagattttcacacaaaataacgttacacgctaccttgtgtggtccagatgttccttccacacgaccgttgcggcctttatacggctgccctgccaggctgctggctgaataaccaatccgttcgtccggttcaatggcggccatcaggttctgctccgcaatgtactacgaatgcgcttcctgtcctggatggccagcatggtccagaacatctccatcatttatcagcaatcttcctgctcatttcatcccgccatgcagcagcagaggattcgaccatgtcatggggaagcacatcaccgcattcgagttgctgacggcctgccgcaacctggtcacggaaccaagaattgtgctgccgttcaacggtctggcgcataaaatcccggattaactgagagccattacggtccatgctttttgcagcggccataaaagcatccttcagttctgcgtcgattcttacactcatattaacctgtgccatttgccacctcgtcattaccacgtatatacacagtatataactgttcagatatcgtcaccaggatatgccgcgccagcggcatgtaaggcggcactccgttgttgcatatggcagaggagtaaaaccgccgaagcccggcgtaagccggtacgtattgacaggtttcacctgtcctatcactggcgctgacagttttatgcgatgtgattatgaatggtgcagttgtgagccgttttcagacaatcgcagggccagtacctcgtcagtcagctgacggtaaatctgctgttcagtctcacgcatcacctgtgcaccggcttccctctccgcatccgcatcaccgctcagacctgatgctttcagccggtcagccaccctctgagggtactcattctccagcatctcatatttctgctcttctgccagcgcccagcggtcagcttccgtacgcttcagtacagcatgccatggtccccagagggagaaccagtccgtaaattcattctcttcacggcttctgaccgtggcttcggcagtgcggaggtcatttgctgtcactcccgacacgccatagaaacgcatttccttcacggcagtgaagagctgaagtttctctgcgagcatggtctggaaggccaggtagacttctatctcatccacaaaatggagagttctgactttatcccgggcaatgtcctccagaatttcgaggcggaacatttccctgcccagggagagcagagcgccggtatcattatcgaaaaggccttctgatgcctgatggaccaggagggttttccggagattgttccatgtgagcgcgacacggtcctcacagctctcagtggcatcagcagcaacagcgaaagactgctgtcgaagctccgcagaggcactgagtttttccagccatgcagcgacctgttcacggaatccggaggtattgcgtgcagagacggtatcggaaaggcggtcaaggaacgcggaaaaggtgttggcatgctcttcatgttcaaaagcatgccatatctgtgatacatcagattgtttgttttccgggaaccatgctgtcacggcatcagccagggggcgatggagtgtattctgttgtccgtcactcatggagaagtaaatccgtgggccgtggtagtccggcgaagaggttaatctttgcagggcttgcagagcatgggatgataatgggttatcactaatatgtattgaacattcattcctcagattaagaatgctttccgggatatgacttatctgatttctatcaaaaaaatactctctgacaacgggattatttccctcagaaaatgctggtagctcagtgaggaaattacgggtcgctctgagatttttaagagcctgtgggagcgatggtaaggagactatttcgttatagctgatattcatagtcaataacgaatcgggtaaagctggcaggttttccagctcattgtgttgtacctgaagtgactccagcgcaggaggtagcacgggcaattctgataatttattgctggcggaatataatttttttagcgtaacaggcagttcaggtaggttagttaattgattatatgatacattgagcagcgttatctgagctggtaagttgtcaggtagcgaggacagatttaaacggtccagtcgaagttcatcggaattattgataagacattctttaagtcgggagacagcctcatcacgctcttcaccggggagcgcttgtttttcccatttatcccatgctgaaaagtaatcagcatatgtaccggaaatagtgttataaaaagaattttggggcaatgaaaagttattatttatcggtaacatgatgggcatccagttaaagccggtggacaaatggctttgttaaaagaaataagtaggagtttttatgaagttcattccatgaaatgaacttcagcacttttttattataataaggataaacaaaattggctgtaaaaaaacgatactgtttcagttaaccgtgaatggcctgtttgcaccgtatgtgacagtaatacgctgcttcatgataagccctcaaaaacatacttatgatatgtatatgagtatatattaatggtacaaaccatcaatatccataaaaaatacaccattaattcgctataagtttttaatacaattttttctgctgcgatttttttatacgataattagcatacttataacaccgtaagtaaattacatgaaagcaggcacagcgtgatccgatttttctgcgtataacgattttccggaagtgtaatacggttgcgccccgggccgggcccccttttctgttgttgtgtacgggcccacagagaatggtaccggaggcgggctcagaagtactgagtcgggtgatcgcgtcaggccgcgaagtactgacacgggtattggtgactggtgatgaagtactgccccgggtatgatttcaggctgattcaggttctgattctctgtcagtccgcgtggttgctggcctggctgatttcctgttgtcaggcgttcaacaaccgactgcaggttatagcccggttcacgaacacctgacggcaggtatcactcacccggtgaaggtaaatccctcaagaggcagcaactctgccagtcgctcctccggccattccggcagacgcgtcaggacgtccgtcagccaggcatgtggctccagaccattacgttttgctgtttacagaaggctcattatctgtgccgcccggtttccggccatcagtgaaccggcgaacagccacgctttccgtcccataacaaccgggcgaatggcacgctcgcagatattgttgtccaggggcaccgctccgtcacccaggaagcagctcagttcatcgcgccggttcagaatatagttaatcgccttatttaatggcccgtcgggcggacagcacggctcctgctcttcaagccaggcaaacaggtcattcactatcggccgggagtaccgttatcgccactgccgtattttttccacagggcgctgtcagcgccagtgatataagacggtaattcgccatttggattgtccgctccacccaacatgttgtttccttaaggttctcacaccagaaaggacatcaacatgctgagcagagaggacttttacatgataaagcaaatgcgccagcagggcgcgtacatcgtcgatattgcgactcagattggttgctctgaacggacggtcagacgctacctcaaataccctgaaccgccagccagaaagacccgccacaaaatggttaagctgaaaccgtttatggattacatcgacatgcgcctggcagagaatgtctggaatagcgaggttatcttcgcggagattaaggcaatgggttatacgggcggacgttccatgctgcgttactacatccagcccaaacgtaaaatgcgtccgtcaaaaagaacagttcgcttcgaaactcagcctggataccagctccagcatgactggggcgaagttgaggtggaggttgccgggcaacggtgcaaagttaactttgcggttaatacgctggggttctcccgcagcttccatgtcttcgccgcaccaaaacaggatgctgagcatacctacgaatcactggttcgcgccttccgctacttcggtggttgtgtgaaaacggtgctggttgataaccagaaggctgcggtgctgaagaataacaacgggaaagtcgtgttcaactccggattcctgttgctggctgaccactataacttcctgccacgggcatgccgtccacgcagggccagaacaaaaggtaaggttgagcggatggtgaaatacctcaaggagaacttcttcgtccggtaccgcaggttcgacagcttcactcatgtcaatcaacaactggagcaatggatagccgatgtggctgacaaacgggagcttcgccagttcaaagaaacgccggaacagcgcttcgcgctggagcaggaacatctgcagccgttaccggatacagacttcgataccagttacttcgatatccgccatgtgtcctgggacagctatatcaaggttggtggtaatcgttacagcgttcccgaagcgctgtgtggtcagccggtatcgatacgaatatcgctggatgacgagttgcggatctacagtaatgagaaactggtggcctcacatcgcctctgttcagcatcgtctggctggcagacagtgccggagcatcacgccccgctctggcagcaggtcagtcaggtggaacatcgaccactgagtgcctatgaggagctgttgtgatgcatgaactggaagtcctgctgagtcgcctgaaaatggagcatctgagttatcacgttgaaagcctgctggaacaggcagctaaaaaagagctgaactaccgggagttcctgtgcatggcgctacagcaggaatggaacggcaggcatcagcgcggtatggagtccaggctgaagcaggctcgtctgccgtgggtcaaaacgctggagcagttcgactttaccttccagccgggcatcgaccgtaaagttgtccgggaactggctggtctggcgttcgtggagcgcagcgaaaacgtgatcctgctgggccctcctggtgtcggaaaaactcatctggccatagctcttggcgtgaaagcggtggatgcgggacatcgggtactgtttatgccactggacagactgatcgcgacactgatgaaagcgaaacaggaaaaccggctggagcgtcagctacagcaactgagttatgcccgggtgttgatcctggatgaaataggctatctgccgatgaacagagaggaagccagtctgttcttccggctactgaaccgtcgatatgaaaaagcgagcatcatactgacgtcaaacaaagggttcgcagactggggagaaatgttcggagatcacgtactggcaacagcgatcctggatcggctgctacatcactcaaccacgctgaatatcaaaggagagagttaccggttaaaagagaaacgtaaagctggagtgctgaccaaaaacacaacgccaatcagtgatgatgaaatggtggaaagcggacagcatcagtaacgaaagtatttagcgggcatgaaaatggcaaataacggtcaaacatcgtggcgttgacagcgctcacggatgagtttctcaatgcggtaaagacccgcgatttttctcagctccacgcctgcacggggctccctgctggccttatacagatttgcgaagccgcgccctgcgtgactccagcaacaggctgatgtgatgcaggggtgattatccgccagagatttgtacacgctgtatccgtcactcaccagcgtaccgccacaccagcactgcagccaggtctcagggtatctgagggcccgtccggtctgcgagtcaaaacagacgaccggcggaccgcttctttcaccgctgacgtaagcacacagccagccggagcaggatttaccgcctttccgggtatcaaggagcctcagggacgtttcatctgcatgaataacatcccgtgtcagaagctcatggcgtagcagtttcgccagcggggccagtgcagctccggcaacaccgaccatatccgccattgtactgacggggagctccacacccattcgggagaaaatgtgctgctggcgatacaggggcagatggtcggtatatttgctgatgacaacctgggtaatggctccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgtgcagttgcccggcatccaacgccattcatggccatatcaatgattttctggtgcgtaccgggttgagaagcggtgtaagtgaactgcagttgccatgttttacggcagtgagagcagagatagcgctgatgtccggcagtgcttttgccgttacgcaccaccccgtcagtagctgaacaggagggacagctgatagaaacagaagccactggagcacctcaaaaacaccatcatacactaaatcagtaagttggcagcatcacccccgattagttgttaatcatatttaatcatagtgattactttcagacggaacaaggcaaataatgcgagtcagcgcatataaggcggcatacggtggttacatgtagcacaggtgtaaaaccgccgaagcccggcgcaagccggtacggtcgtccgaatatcaccagtccccctccggctctgcgaggccatgcggcctgtcagcgatgaaaatgcctctgtgcgcggtcggagtggtcccgacaagggtttacccgcagtcggggcgtatctccgcgttagcgggccgtgagggccgcttacgagcgtgtatgagagacgtacagtagttttgtccgctcacaccagtacaaaccagtcgaaataccggtaacgacgattactcttgtctctgatgccggtggactacgagattttatttactggcgtcctgatgcggtcaggactggtatggaacctgtttatgtgatacttaatgctcttctggattccgggcgatttacgcgtaaacttattctgttagggctgtcaggctcattttcctatatcttcggctcaattgttgcgacactgggaatgggacttgttgtcgattatctgggatggggggccacatttatcgtactgatactgtctgctgtttttgccatcattttcacactgatgagtcgtgaacgctcactggagttcgaaaaagagtaattttttcaggaaaaacataaaattactgactaaatatttattttttgaatcgcagggcattccggataaggttgatatcgtgtcacggctgaagacctatggatacagtatttctggtgtggagacggatgacggatataaggcgcttgtccgcgcttttcagcttcatttccggcagaaaaattatgacggcataatggatgctgaaaccgctgccatcctgtatgcattgctggagaagtatttcccgggtaaataaggagtgagtcgtcagagctgcgatatatccccttcctccatgggctgtctcttatacacaaatccccacccggggatttgtgatgtcagatgtcccctgtaacactatgttttcattgcgctctatgaagctgagtgagcgtaacgactggagctgcttctgcggtgctccggtggcgaacgtgaagtcatactcttcgaacattttcaccgccgggaaggctaccattcgggtatacatcgcctgtttacgttgatgacgtgccagtttttcttcatgaagcatgatgcttcaagaagggcactgttgcaaagttatcgatgaggtagctgaccatcactgctgtcggaatcgatcttgcccttctgtttccagtcgcctcagcaaactatttttcagttaccacctgtcctgcggtaagcgtaaaccgaccaccgtatgtagccattagacaagaattggtaacttagacgcccatctgacatagacgaacacctaagtatggaattacaggactggcgaaaagaacctcgtaaaaagtattcgaatgaattcaaacttcgggtgatgtcttcaatcgacagaagtttctggtaatctactggtatttaaggttttccatggctaccgttactgttcattgtccgcgctgtcattccgatgaagtttatcgacatggccgtagttgttcccggcatgagcgtttccgttgtcgctcatgtaaacgtgttttccagctcacttactcttatgaagccagaaaacccggcgttaaggagcagatcgttgaaatggcacataacggcgcagggggccgtgataccgccaggacactgaaaatcggcatcaataccgtcatacggacattaaaaagctcccgccccggcggataacatctgcgccagtcgctcacgctgatgttgcccttatctgtgaacttgatgagcagtggcgcttcgttggcagcaaggctgcaacaacactggctctggtatgcgtacaacactaaaactggcggtgtgttggcttacactttcgggccacggaatgatgagacctgtcgggaactgttagccctgtttactccgttctgtatatattaggccgttggtggtaagtggatcactttttagactcgaatagctgcagttcacaaaaacaggaatccggctccgttttttgttgtcatatcagagggatatttgttatgaagaaaatatagtgagtatatatactgtcaggatacaatatgattaatggggtgtcgttacagggaactgctggatatgaggcacatacagaggagggaaatgtaaatgttaaaaaattgctggaatctttaaactctaaaagtcttggtgatatggataaagattctgaattggcagcgactttacagaaaatgataaatccgtctggtggggatggtaactgttcagggtgtgcgcttcatgcttgtatggctatgttagggtatggagtaagagaggctcccgtacctaatgaaatatcagagtatatgaccggattttttcataggcatttagagcaaattgatagtgaaggcatagtgtcacatcctaatgaaacttatagtaaatttcgtgagcgtattgcagaaaatattttacaaaacacttcaaaaggaagtgtagtaatgatttctattgaacaggccactcattggatagctggatttaacgatggtgaaaaaattatgtttttggatgttcaaacaggaaagggatttaatctttatgatcctgtagaaaaatcgccagatgcatttgtagatgaaaatagttcggttcaggtaattcatgtatctgatcaggaatttgatcactatgctaacagtagtagttggaagagtaagaggctttgctgatgctgagagcctgtacatagatttgagccggccccttgaaacgccagacagtggatgtatcttaaaataagagataggcttggatatatgtctaacactagttctaattttgagatgactggaactttgctggggctggagcttcgtaaacgtaaaactcctcaggaaaaaattgccattattcagcagacaatggagcctggcatgacggtgtcccatgtggcgcgtttgcatggtattcagcccagcctgttgcttaagtggaaaaaataatatctggaaggaagtctcactgctgttgctgccggagaggatgtcgttcctgcctcagaactggctgccgctataaaacaaataaaagagttacagcgcttgctgggtagactggccccctgaatctccagacaaccagtatcacttaaataagtgatagtcttaatactagtttttagactagtcattggagtacagatgattgatgtcttagggccggagaaacgcagacggcgtaccacacaggaaaagatcgcaattgttcagcagagctttgaaccggggatgacggtctccctcgttgcccggcaacatggtgtagcagccagccagttatttctctggcgtaagcaataccaggaaggaagtcttactgctgtcgccgccggagaacaggttgttcctgcctctgaacttgctgccgccatgaagcagattaaagaactccagcgcctgctcggcaagaaaacgatggaaaatgaactcctcaaagaagccgttgaatatggacgggcaaaaaagtggatagcgcacgcgcccttattgcccggggatggggagtaagcttagtcagccgttgtctccgggtgtcgcgtgcgcagttgcacgtcattctcagacgaaccgatgactggatggatggccgccgcagtcgtcacactgatgatacggatgtgcttctccgtatacaccatgttatcggagagctgcccacgtatggttatcgtcgggtatgggcgctgcttcgcagacaggcagaacttgatggtatgcctgcgatcaatgccaaacgtgtttaccggatcatgcgccagaatgcgctgttgcttgagcgaaaacctgctgtaccgccatcgaaacgggcacatacaggcagagtggccgtgaaagaaagcaatcagcgatggtgctctgacgggttcgagttctgctgtgataacggagagagactgcgtgtcacgttcgcgctggactgctgtgatcgtgaggcactgcactgggcggtgactaccggcggcttcaacagtgaaacagtacaggacgtcatgctgggagcggtggaacgccgcttcggcaacgatcttccgtcgtctccagtggagtggctgacggataatggttcatgctaccgggctaatgaaacacgccagttcgcccggatgttgggacttgaaccgaagaacacggcggtgcggagtccggagagtaacggaatagcagagagcttcgtgaaaacgataaagcgtgactacatcagtatcatgcccaaaccagacgggttaacggcagcaaagaaccttgcagaggcgttcgagcattataacgaatggcatccgcatagtgcgctgggttatcgctcgccacgggaatatctgcggcagcgggcttgtaatgggttaagtgataacagatgtctggaaatataggggcaaatccaaattagtagaacatatgtttttcgataaacgcaccgataactttttgaggtagcctgagtttaacggacactccttcctgaaatagaatggcatcagaaggagccaatattgacgggtaaagccctgtttatggtgctcaggagccagcgcccacctgccgggctaatccaccactccgatcgaggttcacagtactgcgcatacgattaccgggtcatacaggagcagtttggtctgaaaacatcaatgtcgcgtaaaggtaactgttacgacaacgctccgatggaaagcttctggggaacgctgaaaaatggtacagggacggagtaaaccgtagtagcggtgaaggaccgtaatgggtccggagcaaagggcttgccccattgtgctgatggtgcggcggcaaccacgggcgacaatgccgatgggaggactgctgtaaaatcggcaaagccatttcctgtaagtaaacgacaggtatgggaagcttataaacgagtaaaagccaaccgtggggcggcaggcattgacgggcagacactggccgggtttgacgagaatgttacagacaatctctacaaactgtggaacagaatggcttccggcagttatatgccgcaggcggtaaggcgtgtggacattcccaaagctgacggtggtgtacgtccgctcggtattccggccgtgtcggaccggatcgcccagatggtcgttaagcagatactcgagccggtactggaacccctgttccatgcagactcgtatggttatcgaccgggtaaatccgcacaccaggcgattgcgcaggcgaggaaacgctgctggaagtttgactgggtggtggaagtcgatattaaaggcttctttgatgatatcgatcatgacctgctactgaagacagtgcagcatcatactcaggcacgctgggtggtgatgtacattgagcgcaggctgaaggcaccggtgcaaatgccggatggagcaatgctggcacggggtcgaggaacgccgcagggcggcgtaatctcccccctgctctcaaatctgttcctgcactatgcctttgatatgtggatgcaacgtcagtttcccggcgttcccttcgaaaggtacgccgatgatgttgtatgccactgtcacagccagtggcaggctgacgctctgataagtggactgaggcagcggcttgcgcaatgcgggctgcagctgcatccgcaaaaaacacgaatagtttactgtaaggatgctgaccgacggggggactacccggaaacgagcttcgactttctgggctatacattccggccgcgtctgtcgatgaatcgctggggtaagaccttcgttaacttcagtccggcgatgagtgccagagcaggaaaagcaatccgtcaggaggtgaggagaatcgcagtgacaagtccttgtacgagctggcgcatatgttcaatgcgaaaatcagaggctggataaagtactacggtgctttttataaatcagcactgtatctgacgctgaggcaaatcgatcgcaaactcgtgttatggctgccccgtaaacacaagcgacttaggggccaccgacgacgggccagccactggctggcccgtgttgcgcggagtgaaacgaggttgtttgcacactggccgttactatggggacaggcctcaatgagaagagccggatgagctgagaggttcacgtccggttccgtgagcaccagagggggaggttccctcgggtgactcgacttgtagtacattgttcacgaatgagtgatgcaacaaggctgaagaacaggttatcggtgcgcttctcggaagtcgggttggtcctgaacgcagggaagacgaatatcgcctacattgacacgtttaaaaggagaaacgtcgctacgagtttctcctttctcggatatgacttcaaagtgcgtactctgaagaatttcaaaggcgaattgtacagaaaatgtatgccgggtgcatcaaatgcagcaatgtgcaaaataacagaaacaatcaagaagtggcgtatacatcgctcaacagctgagagtttgctggattttgcgaggcgctacaatgcgatagtgagaggctggattgagtactatggaaagttctggtccagaaacttcagctatcgactgtggagtgcaatgcagtcacgtttgcttaagtggatgcagtctaaatacagactttcgaaccggaaggctcagcgaaagctggcgctggtaaggaaacagtatccgaagctatttgcccactggtatttactacgtgcatcgaatgagtgatcaagagccgtatgacgcgagagtgtcacgtacggttccgtgagcagccggagagtgaaactctctccggcggggttccgatagcggtggcgaacatgcggcggtgttgtactcgctgatcggcacatgccgtctgaacaatgtggagccagaaaaatggctgcgttacgtcattgagcatatccaggactgaccggcaaaccgggtacgcgatctgttgccctggaaagttgatctgagctcttagtaaatatcaatacggttctgacgagccgcttacgcagatcatgccataatgaataaactttaatcagcgaaattgacgaagaagctggcagcgctccaacatcagttaagccgcaaggttaaattcagcaacaactggcagaagcagaaacacaaaatacagcaactgcattcccatatcgcaaacatccgcagagactactttcacaaagtcacagcgactgtcagcaaaaaacacgtgatgatcgtcaattacgttatcactgccaactgacttcaccttttggttcatatgaagcagcatccagcggtgaatgttcccggatatattcgataagtacttcagcatcaataaatccggtatttatgtatccgggtctattatcaatacgtggatagccatccccaccagtagcattaaagctaaatgttgtcatacgatattttttgttaaggtcaaccgatttaccttttatttttagcatttcgatttttccttttttgactacaaagctggtattcaaaaattgtgggtaggctccggaatccggtttcatctgtgccactgtagctaggtagtcagctacttctttccccgtcaaatcaatacttactaccgtattaccaaatggctgaactttcagtatatctttgtatgtgatgttaccactctctatggaatctcgaatttccccaccactgacaacagcaaaatctgcgtcaatacgttctgtcagagcgctcagaatcagatggcccatactggtctgaacaaaacgaactttacttctgtccccttccagacgtccgttcacaaccccaaccttcacatccagttgtgctttgcctttattctggaatggtgtcagtaaggataacatctgtggatcttcagtaatttcgggggtgtaaaaagaaaattccgtttttccatcttctctggtaattctcattttgagattaactgggtgtaactgataattaaccagtttcattgtaccgttacagaattcaaaatcagcctgaccgacatattttccccactcatgcgcttgtacaatccagatgccattttgtttatctggtacacaggttgtccccggaatataattccactgttttttattatcgctggccatgcagacaggagcttgtgaatgtcctcccacaatcattgctaatgagccagtcggcaaactgcgagccatttcaacatcaccaggagcattcgaacctgactcgccattgtcatagtgacccatatgtgttgccgcaataataatatctggcttttcctgctgattaagttcatcaatgacactcctcgcctcagcagcaggttgacggaactcaatatcagtaaaatattcagaatttcctgttttcgcagtatcatctgtcgtcaagccaatgactgcaattttcaaattctgacggatgaaaattttccatggactgaataaacgcctccctgtacttttttgatatatgttagcagaaaggaaaggaaatgtggcccatttttcctgctgacgtaatatatttagaggattatcaaattcatgattaccgacggccattgcatcataaccaattagattcattccacgaatatcgggttcagccttctgaaggtctgattcaggtacaccggtgttaatatcaccaccagataataatagaacacttccgccattatttattactttctgccggatattgtccaccagagttttttgggaggataacccgtactcaccatgattgttacgccaaaagtgtccatggtggtcatttgtatgcagaacggttattttatatactttatctgtttcatacgctatagcaggatttcctgtcagtaatgacagggtgaacattattagcgtaatgtttttttttaacggtatcattgtacctgtatgaacgcatccccgtttgcaaggcttccgttgttctgacatcgacgagtagttgctgctctgtatccggccttattgcagtcagagctgcgggccactataagattcgctgacctgagccttattcccttcacggactttatgtctaggacacaattcaggtttatcaggtcacggtcttacctgtcgttgtcatcacgtcgatcaccatagccccctttcagcattcacccttctataaggtttgatgtttatcagtttcagcaattaatatggtctgatactgacgtgaaccgccccaagtttcctggagaatgttttatctgtgaactcaggctgccagatcatcgtttccgatagaagcataataagctttttctgcttctgccggagggatgtggcccagcctttccagcaatcgtcgattattataccagtccacccacgtgagtgtggccagttccacttctgcacggtttttccagctcttacggtgtattacttccgctttgtaaagaccattgatgctctccgccatcgcgttgtcatacgagtcgcctgtacttcctgttgatgccagtaatccggcttccttaagccgctgtgtgtaggccagcgatacatactgagaacctttatcactgtgatggaccgtgccggacggtcgacgggcccataacgcctgctccagtgcatccagcacgaatgtcgtttccatggacgatgagacccgccaccccacgatgtatccggcaaacacatcaatgatgaacgccacatagacgaagccctgccatgtgctgacgtaagtaaaatcagccacccacagctggtcaggtcgttctgccacgaactgacggtttacgcggtcgcctgcggcaacggctttccggctgatggtcgtacggacctttttaccccggagaacaccggcaagtcccataaccgccatgagacgtgccacagtgcatctggccactctgataccttcccgtaacaactgacgccagactttacgcacaccgtataccttgtgattttcatcgtatacgcgctgtatctctttcttcagccagtcatcgcgctgcgcacgggcactgcgtttatccggatgatgtcgctgttgctgacagtggtaatacgttgacggggcaatatgcagttcgctgcatagcggtccgaccccgtactgctcacgcagcttatccagcagtggcatcattttttccagaggcggtcaaactccgccttcgcaaaataagcggaagcctggcgaaggatatcgttactgcggcgcagttcacgattttcacgctccagctctttcagacgctgacgttcagcggtggtgagccctccatcaccgcccccggtatcccgctcatgctggcgaacccggacacgcagagtctccggcgtacagccaatctttggagcaatggaacaaattgtcgcccattgtgagtcatattcgccctgactttccagaaccatacggactgcccgttgacggacttcgggggaaaaacgagtatttttagtcatcctgtttacctctttctcaggaagtttagtctccaggattcccggggcagttcagttcaggctgtacgcttccccatgtaatttgcctcgccacttcaccgggctcaggaccccgtaggatcttcaccgaaaaaaatgcatcaccagttgctttattatttgttttaggtagacctgttactaatttatacctactctgaccaagtctggataagaaaggcacacctgcacatttctacttggaaatgttttcttttaccattttgctcaaaaatttcaacatccaatattcctcgtacagaactactcaagtcctgaatagaaaaagttccagctggtacagtagagtcatatattactcgaccttgttgagatacaacaacacgtgcattagtttctgcaataccagtaatctgaggcgcataaccacgaatatttggcggtagcattcgttcatcgctttctaatgcaattcctgtgtaacgccaagcaacagcgcattctggcgcatgatctggtaaacacgtttggcattgatcgcaggcataccatcatgtgcacaaaaacccaccgagcttttacacggtgggtcccagagaaatgcaggacatcaacacgccctgcatttttattatcagaaggtatatttcacacccaaaataccttgggtgtctctgtaactgttattacccaattgctgagatacgtttccccagatgcttaggttatctgcaagacgcccccccacaccagttttgagttcaatcacattacggttgcctatctggtgatttgacatgccattcataattacaccatattgctttgagctccatttccaattcacttccaaaaatggttcgaacttacgaagagtctccttatcgatacaactctgactaattaaggatgtacgcaatcccaattttgcttgaatattattgaccccagaggatgtgaccatagtgccattgtgttcaatatagtcgtttgcttttaccccatgcctagttatctggacttgtggattgagatacaatgcgttgtcaatattattatgagcagtccagcgtatggttgggtagatatagccagcttccaaagctcctgttattcctttgctggaatatttctcaccagttaacccatctcctttgactgtattattaaaccaaccgtactgcaaccaagcatccatatatagacctgttcgttctttttcattctgaaaccaagaactgtacaaaccggcagtataaccgctcacagttcctcgtgaactataactagtatacttattatgagttttgctatcctgatatccataagctcccataatacccacatgcaagccatcatgatcagtgaagtttgtcttcaaaatatctccaccaatctgataaaccatcctgttagctgtggtttttaattgtctatcagctaaattatgctcattatgtccaccaacagtacgaatccacatggtggtttcgctgagctgacctgtagtaggatcaacaatagtgctaccgtgtcggtcatagagagaaggactaaacatcgtgttcattgctatcaggttagcaatataactgccagcttcagggcgccatgtgtgaatactaccttcacccaaattaagtgaagatacaagctgtgaactagctcggcgactactcatttgagtagactcttgattgctcatctgttttgattcttgattatccatctgactagttagataccacttattggtattcagtccagatacagtgccctgtttcaggcgatagtcataactaccagcaacaatacggcctttctgaataaaagcattcttatcagaagaatctgtcgaaataatttgaacaccttcaagagttttatctccagagcctccctcatttgtcactgcaacataggtaataccagacgcatggcctgcaatttccagccggtcagttttactgttgtcattgccgagaaccgcgcccaaagataatattccgccctttccatgccaattacctttctgaacataggtctgaccgacatttgacgaactattattgagaataacatgaccgtttttctccagagtcatgttacctgtgacaatgacggggtcaggcaaaaaaggggcattaatattattgattagcacagtcccactatttaataaagtgttaacagtttggttcatgcctgaaaaattcaaggtagcacctttattaacaataacaccagcggtacgtgtcatagcttcaactgtccccatttttagaatgcctgcattgatgttggtgaaaccagagtaggtatttttttccgccaaaatgagagttccctcccctgattttgttagggattttccgtcccagctatttatattatgatggctgagattatctgccagaatagtactaacagtaagttcagcgttttctttcagattgaactctccatgactgtcgccatctgtatcattccagctcaaaccgtagatcaatttattctggtcactgatatagctttttgtatagtcaatagcaccagtagtgagtaacggattgatgatagatatattagaaacactgaaacctgttatccctttatctgcacttagcagtttatagtccttctgcgcccagttatttataatagcttccacttttacattgttgccaaatgacatatttccagaagcttccagactactgattccagctaaatttaaagaactgttctcaccaaaagaaacgtcacctgaaacagtgaatttgttacctgatagtgaaacagttgtacaatcaccagcacataaatcaccttcaatagttggtgtggtcccagtgtaatcattgttgataattttcagaatggatgaattgttaacctgtacatcaccagtaatgacagaaccttcatggagtattaatgaattgtttccaccagtgatgtggatagcattacctactacattttgtgttccatcatcttctttacctgaaattatatatccgctattagcaatggtcatattgctaccatttatagcatccccccaatgtccaccattaccgcctaaaatagctccattgttgattatagacaggttatctcctgtgatagcattaccaccgtagccatcataaccataaccgtaagcagcacctccgttgcctccagcgaacgtgcccttattgattacagagaggctgcttcctgtgatggcatcacctccattaccgccatcaccctcaccataattgttacctccattaccaccggaaatagtccctttgttgattatgggtagattgactcctgtgacggcatcacctccattccccccatcactatcactataactatcgccaccatgtccacctgaaataatttctccatttattataaacagattgtctcctgtaacagcctcaccaccatcaccgttatggtcagcaccgctaccgccgctaccaccaagaatcatgccttgattgattatagacaagtcagatccggtaatagagtcaccaccattaccgccacaactattaccatcattattatcaccagcaccaccatgacctgcgctaatcataatatcagagttaatccgtagcttagttctagatgcatgagaggggttggaagaagttatataccacgcatccattccatcttcaccagctcccaagggagaaagtccatcaacaggtgttaataatttttcataattgtcctctgaaaaatgaagttcttgagtacccgaaagaggaatagcaaaagctattggccccccgagcaacagggatgcaccaacaacaaaaggagaaaaagacaatttgcatgttttttcctttaccgtaggtaattctccggcccccccctgtgaacattgggtcatattacaaaaaaatttgtgaatttgattcatgcactatattatcagtaagtggttgataaacccctaaaaagattccgttttatccggaataaagggacgatatatgcaaaaatatattaaacaaagcctcaaccaaaacatgagagctgtgttatatatcaatgcaacgggagaaatttacctgtgtcttgctttgaaaaaaagagggggaaagctatatgttcgataataaattaataattttactatgatagtctgcttcagaccgcaacttttcagttctttctgtacgcttgcccaaagtttaaccactttgtttttctatacacccaatggttctatctgccccttgatgttaatccccaaagccaataacatgaatgtattcatatcacaactttcctaccagactctccaatgcaataactgcataattgtttttagtggaaatgtatcgttttcttaaagagaagaataacattccattttattatgttcccaatttaattatattaatatatattggcattaataggaaaatacatcaggagaaatcaaatgcagacatcaaacataactaaccatgaaagaaatgactcatcatggatgtcaactgtcaaatcaactacagaagtaagttggaataaattgagtttttgtgacgttttactgaaaataataactttcggcatatattctccacacgaaactctagccgaaaaatatagcgaaaaaaaactaatggatagtttctcaccatcactatcacaagataaaatggacggagagtttgcccacgcaaacatagatggcatttcaatacggctatgccttaacaaaggcatatgttcggttttttatcttgatggagataaaatacaatccactcaactttcatcaaaagaatataataacttgttaagttcactcccccccaaacaattcaacttaggaaaagtacatactattacagctccggtatcagggaattttaaaactcacaagcctgcaccagaagttattgaaacagccattaattgctgtacatcgataataccaaacgatgattgcttccccgttaaagacactgattttaatagtgtttggcatgatatttacagagatattagagcatctaacagcaatagtacaaaaatatatttcaacaatattgaaataccactaaaattaattgcagatctaattaatgagttaggaattaatgaattcattgatagcaaaaaagagcttcaaatgctaagttacaaccaagtaaataaaataatcaattcaaacttcccccaacaagacctatgttttcaaacagaaaaacttttgtttacttcactctttcaagaccctgcatttatatctgcactaacatctgcattctggcaatctcttcacatcacgtcttcctctgtggaacacatatatgcccaaattatgtccgaaaacatagaaaacaggcttaatttcatgcctgaacaacgagttattaacaattgtggacatattataaaaatcaacgcagttgtccctaaaaacgacactgcaatctctgcctctggggggagggcttacgaagttagctcatcaatattaccatcgcacataacatgtaatggtgttggtattaataaaatagaaacaagctatctcgttcatgctggaacacttccaagcagtgaagggcttcgcaatgctatccctccagaaagtcgtcaagtatcatttgccatcatatctcctgatgtttaagccaatattatattggctgccagactgtaaaaaaacgttcttcagaacagctctataaactattaaaccaaagctaaaaggatggggaccaatagaatctcagcccatcctttttatatcattatgaaccgccccggaaatcctggagactaaactccctgagaaagaggtaaacaggatgactaaaaatacacgtttttcccctgaagtccgtcaacgggcagttcgtatggttctggaaagtcagggcgaatatgactcacaatgggcagcgatttgttccattgccccaaagattggctgtatactggagactctgcgtgtctgggtacgccagcatgagcgggataccgggggcggtgaggtgggctcaccaccgctgaacgtcagcgtctgaaagagccggaacgtgaaaatcgtgaactgcgccgcagtaacaatatccttcgcctggcttccgcttattttgcaaaggcggagttcgaccgcctctggaaaaaataatgccactgctggataagctgcgtgagcagtacggggtcggatcggtatgcagtgaactgcatattgccccgtcaacgtattaccactgtcagcaacagcgacatcatcatgataaacgcagtgcccgtgcgcagcgcgatgactggctgaagaaagagatactgcgcgtatacgatgagaatcatcaggtatacgctgtgcgtaaagtctggcatcagttgttacgggaaggtatcagagtggccagatgcactgtggcacgtctcatggcggttatggggcttgccggtgttctccggggtaaaaaggtccacactaccgtcagccggaaagccgttgccgcaggcgaccgcgtaaaccgtcatcaaggcaatatgccgcgtactccgggtggcccgcagcggctggtatacgtggtgtcagcggcggacaaggataaacacacgtcagcagttccgtcagcactgcgacagcgttgtccgcaaggcttttacccagtccagcgttacggtgccccacgcctgacggatgaactgtgtgctctggttaccactttaacgtaaaaaccgtggcagccagtctgcgccgtcaggaactgagcgcaaaagcctcccagaagttcagcccgatcagttaccgtgcacacggcctgcctgtatcagaaaatctgctgacgcaggatttttacgccagtggcccgaaccagaagtgggcgggagacatcacgtactattacagctcaccgactgcggggaaacacggggcgccgggttattgaaggtgacctgtccagctacttcgacacagtacatcaccggctgctgatgaaagccgtctgccgcaggatcagtgatgcacgttttatgagactgctgtggaaaacgccgtgttgatgttggtctcttccgggcggtcagtgaaagtatgccacagggcggtgtgatctcgccgttgctgtcgaacatcatactgaatgagttcgaccagtatctgaataaacgctacctgagtggaaaagccagaaaggatcgctggtactggaatcacagtatccagcgaggccgaagtactgctgtcaaagaaaactggcaatggaaacctgccgttgcttactgttgctatgccgatgactgtgtgccacgaaggcgcgtcctggggacgtgagtccctgtgcagccatgcggcacaagagatgagggaaggcccctcggtgtcggcgtgcaggcgcaggcaccaaaccatccactgctgctgcatttaagagatgtggtggtgagcgagatgggaaaaggtcggacgtgagccgatcaggtatggttcgaagagacgaacgaaagtgaaccgctgatgatgtgtcgaaagcatatagatgatgtcaaaacaatggtggagtcgttcctgtgggatatgtttactgcctgcgcggcatccggcataaaagcggcgtgaagtcgattcaggctcttgtggggaacgtggacctgtcaccccgatgtgaagggagaagcgcaagtggaagccccatgagcgccagagtaccaatgcggggtacagggatggagcaccaagcgattgcgcagtaaacatgcttgacaacttaattggggccccctcttccttttatggtgtaattgtttgattgaatacttccggggatttcagtatgaaatgaagtatatttaatatactttacaaaaagtttgtattagtggaataaggacaatagtttatccaaactgccaagcccctttattataagtaagatctggtactaaataagcgttctaaaccgccccgagtaggcggcctgacggctgcggcttgtcatggcctgtgattaccgttataaaaacagacaatatcattgtctttcaggtagttatatggcccgttcagctaaaccacgcaaacgaaaacctgcacctcaaagaagcaaactcctccgctatgtcgtaaaacttcacgaagatgatttttttgacgaagaagaagctgaggttctgcgctttgataattttgacgatgccgttgagtgttgcgcagacctgaatattccgttctttgtggatgccggaaacaaaaaactggtcttctggtttgtacgtgttgatgacgaagggtatccggaaattgcccgctgcacggagcgggaatttgcgacaattcctgccggtatcagcgccgacggcatgtattgccctgaatgcggtacggttcactggccggatggcgttattccgcctttctgatgcttccccgttttgccgatatttttcagcagggaaaccgatggcttaactggctggagaagcagccggtgcagatgagccgcctggaacattatgccggtcaggatgaaattggcctgcgttacaacagccaccggacaaaacgagaagaaaaccttgtgatgagtggcgacgagtttatggaaaggttctcctggcatgtggcggataaggggttccggatggtgatacggggtcctgagtccggtgaagcggcgattactggaagatgtggtgtacgccataacggagacagtgagaaaaacggcgaggcaaatcacaaggagagggatgtatcagcagtgactgaaggttgacccgctgaagtgcgagctgtgcggaggtcagatgcggtttacggggctgaagaggggttaccgtctggcagagcaggttatgatgcatgagccgctggctcgaatacggtggtgcggctgagagtcgcagaggggaagttgcgtccatgttgaggggaacggagcaaaaaaccatcagagatacattgaatcaatcaatgtaagcaatattaagtgtctataccgattacttgatgatcaggttgtgttgaataaacgcgcattcttcggtgcttttttaatcttttggggatttaaattcctatcgatggacatgaatgcaggctatataagagcagcgcgcatccacttacccaatgcggttgagaaaatagccttcgatcgctttcatgtggcgaaacaaccgggtgaggtggtggataaaacccgccagaatgaaccgccccgggtttcctggagagtgttttatctgtgaactcaggctgccagatcatcgtttccgatggaagcataataagctttttctgcttctgccggaggagtatggcccagcctttccagcaatcgtcgattgttataccagtccacccacgtgagtgtggccagttccacttctgcacggtttttccagctcttacggtgtattacctccgctttgtaaagaccattgatgctctccgccatcgcgttgtcatacgagtcgcctgtacttcctgttgatgccagtaatccggcttccttaagccgctgtgtgtaggccagcgatacatactgagaacctttatcactgtgatggaccgtgccggacggtcgacgggcccataacgcctgctccagtgcatccagcacgaatgtcgtttccatggacgatgagacccgccaccccacgatgtatccggcaaacacatcaatgatgaacgccacatagacgaagccctgccatgtgtcaacgccacgatgtttgaccgttatttgccattttcatgcccgctaaatactttcgttactgatgctgtccgctttccaccatttcatcatcactgattggcgttgtgtttttggtcagcactccagctttacgtttctcttttaaccggtaactctctcctttgatattcagcgtggttgagtgatgtagcagccgatccaggatcgctgttgccagtacgtgatctccgaacatttctccccagtctgcgaaccctttgtttgacgtcagtatgatgctcgctttttcatatcgacggttcagtagccggaagaacagactggcttcctctctgttcatcggcagatagcctatttcatccaggatcaacacccgggcataactcagttgctgtagctgacgctccagccgattttcctgtttcgctttcatcagtgtcgcgatcagtctgtccagtggcataaacagtacccgatgtcccgcatccaccgctttcacgccaagagctatggccagatgagtttttccgacaccaggagggcccagcaggatcacgttttcgctgcgctccacgaacgccagaccagccagttcccggacaaccttacggtcgatgcccggctggaaggtaaagtcgaactgctccagcgttttgacccacggcagacgagcctgcttcagcctggactccataccgcgctgatgcctgccgttccattcctgctgtagcgccatgcacaggaactcccggtagttcagctcttttttagctgcctgttccagcaggctttcaacgtgataactcagatgctccattttcaggcgactcagcaggacttccagttcatgcatcacaacagctcctcataggcactcagtggtcgatgttccacctgactgacctgctgccagagcggggcgtgatgctccggcactgtctgccagccagacgatgctgaacagaggcgatgtgaggccaccagtttctcattactgtagatccgcaactcgtcatccagcgatattcgtatcgataccggctgaccacacagcgcttcgggaacgctgtaacgattaccaccaacctcgatatagctgtcccaggacacatggcggatatcgaagtaactggtatcgaagtctgtatccggtaacggctgcagatgttcctgctccagcgcgaagcgctgttccggcgtttctttgaactggcgaagctcccgtttgtcagccacatcggctatccattgctccagttgttgattgacatgagtgaagctgtcgaacctgcggtaccggacgaagaagttctccttgaggtatttcaccatccgctcaaccttaccttttgttctggccctgcgtggacggcatgcccgtggcaggaagttatagtggtcagccagcaacaggaatccggagttgaacacgactttcccgttgttattcttcagcaccgcagccttctggttatcaaccagcaccgttttcacacaaccaccgaagtagcggaaggcgcgaaccagtgattcgtaggtatgctcagcatcctgttttggtgcggcgaagacatggaagctgcgggagaaccccagcgtattaaccgcaaagttaactttgcaccgttgcccggcaacctccacctcaacttcgccccagtcatgctggagctggtatccaggctgagtttcgaagcgaactgttctttttgacggacgcattttacgtttgggctggatgtagtaacgcagcatggaacgtccgcccgtataacccattgccttaatctccgcgaagataacctcgctattccagacattctctgccaggcgcatgtcgatgtaatccataaacggtttcagcttaaccattttgtggcgggtctttctggctggcggttcagggtatttgaggtagcgtctgaccgtccgttcagagcaaccaatctgagtcgcaatatcgacgatgtacgcgccctgctggcgcatttgctttatcatgtaaaagtcctctctgctcagcatgttgatgtcctttctggtgtgagaaccttaaggaaacaacatgttgggtggagtggacaatccaaatggcgaattaccgtcttatatcactggcgctgacacatgtgctgacgtaagtaaaatcagccacccacagctggtcaggtcgttctgccacgaactgacggtttacgcggtcgcctgcggcaaaggctttccggctgatggtcgtacggacctttttaccccggagaacaccggcaagtcccataaccgccatgagacgtgccacagtgcatctggccactctgataccttcccgtaacaactgacgccagactttacgcacaccgtataccttgtgattttcatcgtatacgcgctgtatctctttcttcagccagtcatcgcgctgcgcacgggcactgcgtttatccggatgatgtcgctgttgctgacagtggtaatacgttgacggggcaatatgcagttcgctgcatagcggtccgaccccgtactgctcacgcagcttatccagcagtggcatcattttttccagaggcggtcaaactccgccttcgcaaaataagcggaagcctggcgaaggatatcgttactgcggcgcagttcacgattttcacgctccagctctttcagacgctgacgttcagcggtggtgagccctccatcaccgcccccggtatcccgctcatgctggcgaacccagacacgcagagtctccggcgtacagccaatctttggagcaatggaacaaattgtcgcccattgtgagtcatattcgccctgactttccagaaccatacggactgcccgttgacggacttcgggggaaaaacgagtatttttagtcatcctgtttacctctttctcaggaagtttagtctccaggattcccggggcggttcagaatgaacatccgcatcttcctgttgaaagtcgtcgtcaggcaaagggaactcgcttcctgtggcagcacagcgataaatggatgaccgaatccaggcaggaaaagctgatatggctgcgtgcgcagatgaagctgacgagcctgtgctgggcgctgaaagaactggcaaaggatatctggagcagaccatggagcgaggaaagacgcaacgactggcagagatggctgtcgctggcggccaacagtgacgtccccatgatgaagaatgtggctaaaacgatagggaaaaggttgtacggtatcctgaacgccatgcgccatggcgtatcaaacggaaatgcggaagcactgaacagcaagatcaggctgctgagaataaaagccaagggctaccgcaaccgggagcgctttaagctgggagtgatgttccactatgggaaactgaatatggcgttctaagccttcccaccatgatcggggaagacccagatatgaacaggcacattcagagttactttaaagagcttactgaacgtctgaggtgttagagccatgactgccgcagcattgctggcggaaatgcctgaatcgagcagcctctcgagacgtgaaatcagcgcacttgtcggtgttgctcaggtaaaccgggactcgggaactctgcgcggtcgacgaaccattttcggtgattgtgctggggaagaacagctctgtacatggcggcgcttgaggcaccccggtttaatctggtgataaaggcgttttacatgcgcctacttgcagccggaaacgccaaaaaagtggcactggttgcctgtatgcgtaagttgctgaccattatgaacgcaatgctcagaaaaaacgaagaatggaatgaatcgtatttatgaaaactggattgtgggccataaatattccaaaaagatagcccgctccgtttattaccggaacgggcgagagcatgacattgtctgtaattataatgagactgctgattctacttatttttaatttcaagaaaagcgttttctacaaagaattatgagatttaatgttttgtttagacaatattatcattctgttcaataatgaaagtcttcatcaacaacaagtaaaaacaacgtccctctcatcgaaccggacttgcagatttcccgcatccggctctcttgcaacacatcagaccttcacgttcgggcggttatggggaaggtccagcaagctgtatgagccctaactgtttgaataccagtagattaccagaaacttctatcgattgaagacattaccaactagcgttactaaatgttgaccaagttttgttcacttgtctggttttgatagtagcattctgcagagtattttgatatgataaatatgcattaacaatgatattattgttaatttcctagcatgaaaattaaaagatcaactttttatctcgaatattttttacattatcagttggtttcttatgaatgacaattcattgcttagaaattcttcattattcatcgcctatatgggatgtgtagggtgggtcagtgcttattcttatggctggggaacctctttttactatggatttccatggtgggttgttggtgcaggtttggatgatgttgccagaagtttactctatgctatcatagtaatgggtatactttttaccggttgggggattggtattttattttttcttcttataaagaaaagaagtaaaatacaagacttgagtttttttagactattttttgctattaccctactgtttttccctgtaatattcgaacttttaattctcaaacaatatttcatacttccattatcactttcatgcattattagttcattggttatttcaataataattcgtatatatggacggatcttttctgtatcatgtttttcagatatcccttttgttagagaacaccgcattaagttaattatggctggttttttggtttatttttggcttttttcctttcttgttggctggtataaacctcaattaaaaaaagaatatcagatgctttgttataataatagttggtattatattcttgctcgttatgattcacggctcgttttgtctagttcatttaaagatgacagtaacagattcttaatatttaatacagaacagtcgggattttatgaaatcaatgatgtttatgtcagaaagtaataatcctctcccacaaagtgaattaaatgaatatataatgcgttcgaagtatatgtattgagctgtcatactgagaaacaaaccttagggcagcacaattaaagccagaaacaactatgcaagaaagttactaaataagtgctcggaacagtaaaacactcaggggggactcagtccggattatgtaatcagataaattgccaaacatcacaaatcaccagccggactgagtgatgccgagcattgcaccaataccccgtgatgaacgatgcctgatgcagaaagctccctataaaacaaagaacaaaaactacgaccggcgattcaccgtcatactgatgctgcatcgtggtgacactgtctgtcactttcatgttgactgaacaactctgctgcgaagaaggtgtaccgccggcgggatttgattgtccggcgtctagcttccacttccctgatggaactgaaagttgttatggccggtcagcttgccctgtccgcaggacatgaggtgctgtgacggacagcaccagtaacggaaagtgacaatgaaaatgaatactgcgctgatagttgcactaatgtgtatgtggtatgcggtacccgctgctgcaaaagagactttactggctatgccacgtaattcaacagaacattgctatgcggagattaatgtgcacggaccatatggggtgtatttccgagtggtcccgcatccccccgggggaaagagctgggtcgaatgtaacagtgactactattactctgataaaccaccgggagtacagatactcggaacccgtgcagggtgtcgggtatacggaatctgcgggaccacctcgacactgcatgtggctggaagaggggttgtgtgtattaaaaatatatgcagtccacgtgggatgataattcacaggatacggaaacgtcctgtagtggctgtaagcgatgagatgtgatcttacccagcaatagtggacacgcggctaagtgagtaaactctcagtcagaggtgactcacatgacaaaaacagtatcaaccagcaaaaaaacacgtaaacagcattcgcctgaattttgcagtgaagccctgaagcttgctgaacgcatcggtgttgctgccgcagcccgtgaactcagcctgtatgaatctcagctctatgcgtggcgcagcaaactgcagcaacagatgacctcgtctgagcgtgaaagtgaactggccgctgaaaatgcccgcctgaagcggcaactggcggagcaggcagaggaactggctatcctccaaaaggccgcgacatactttgcgaagcgcctgaaatgaagtatgtctttattgaaaaacatcaggctgagttcagcatcaaggcaatgtgccgcgtgctccgggtggcccgcagcggctggtatacgtggtgtcagcggcggacagggataagccctcgtcagcagttccgtcagcactgcgacagcgttgtcctcgcggcggcctttacccggtcaaaacagcgttacggtgcaccacgcctgacggatgaactgcgtgctcagggttaccactttaacgtaaaaaccgtggcggcaagcctgcgccgtcaggggctgagggcaaaggcctcccggaagttcagcccggtcagttaccgcgcacatggcctgaggtgtactggcaatagcggacaccaccatttgttctttttctaagcagtcatctgataatattttatcctgaaggctgccggggagatattccccagacgagagtgacgacgctgacgattgtagaaaatctcaatgtattcccgtattactgagatggcctcatcccggctcttaaaacgatagtggctcaggctttcatttttcagcgttccccagaagctttccatcggagcgttgtcgtaacagtttgaggtagcctgagtgcgtattccgctctattcgatcacggattggcatcgaagttcgatcagcgattcgcatccattcgatcaaccgggaacttaactttttctaccgctttttaactcgttttttcgctgctttccactgtagggggatttttcctcagggactctccctgaaggaccactctgtgtgagttatgtaccagtctgtccaggatggcatctgctgttgttgggttctccatcagaccatgccatttatctaccgggaactggcttaccactatcgtgctgctttgtccgtaacgatcctctgtgatctccagaaggtcattacactgggcgttacttaactgttccagccccagatcgtccaggattagaacctgtattttttgcaactgtttcagatatttcagccaactgccatcaacacgtccctgagccaactgttccagtaatcgcccggttctacagtaccggacgctgtatttctgtcgacatgcctgttcgcccagcgcattggctatccagctcttaccgcatcctgtcggaccggttatcagtaggtttttcctgtggattatgtagtggccgttcagcagttccctcatctgttcagcccgcagacctcttgaggccggataacgcagcccttccggtgttgcctgatatttcaggcaggcattttttctcaaccgtgccacacgactgttttctctgctcagaagctcgtcatcaaccagcagtgtcagcctttcctcgaacgacagttcgtcgtaagtctgaggcgtttcccgttgccgtatcaggctcccggccatcgccgggagtttcagttgggtcagtttgtttaacagattatcactcatacggttgttccttctgtcagtggtagtagccaggaccacgcaggttttcatgctctgaagcatatgccggatgttgttcgccctggcgggataacggaagctcaacgttatcccgatgatgacgcagcagattgtcgataaagcgtctgtccggggcgttatagtgcagggcaacgtaacaggccttttccagccgttccgggccgtattttttctgaagtgccagcagacccaggactgcacggtaagccagctccggatggggtttggctttcagcatggattcaaccacccggcctgtggacggacctgtacggtttccctgctcgataaggcgttccggtgtccatgtcccatgccgccggtggcgttctggcatgtgaagcggattggttgtgtgtttatattcatgtgtgctgcgcggatgttgcgccacacactgccccttgtgccagagctgtaccaccgactgccccaccttcagtgacagacgctgaccgaccagttcgtgtggaacggagtaccagtgacgggcatattcaacgtggtagtcagggccaaccttcacggctttgtattccgtgtattcataaggctccagaggcagcggagagagtgctggtgcatccagcatgcggaagcgttctgcgcgggtttgattaccgtatcccttcattggtcggttattcatgtccgtcagcaactcccgaaggcgtgcattcagcgccctgagcgtgtggaaggtttcattacggatgcgggcaagaagccatagctcgacgatgaggacgccgttttctgcttttggtttatctttaggtttgcgcgggcgggccgggataataaccgtgccatagtgctcagccagcgcctgataactgtcgttgatgacaggctcatagcgatcggcttttttcactgcgcttctgaggttatcggggatgagcagcttcggtacgccgcccaggaaggtcaggcaacggctgtgggcatttaaccatgacatcatgtcctgaccttcgcacgcttcaacataggtatagttggacgcccccattacagccacgaagattgccacgcggcgtatctctccggtgtccggattaataacagggaccgtaggaccacagaagtcgataaagagcttttcgccagcacgatgctcctgtcgcatggagaggcgtcgcgtttttttccacttccggtagcaacggcagaagtgtgagtatcccatcgctttgtcacccgcctgtgccttgtattccatccacaacagttggcgcgtcacgccaggttttctcatttccgtatcgaagtacagcatgtcaggcataacaagctctgacgcagaggtgtctttgggcgggaaaatgagtttctccagcgtgtcatgggatatatccgcaggtaatggccatgacagagaagaggctttgaagcgggcaagaacctcaaaaacggttgaggcgctgatgtgtaacgtcagtccgatacaacggttagtcagcttcttatcgaacttgagctgaaggatcttaaaacaagtttccatagtgctcctggattttctctttttggacattgacgtctccactggtttaaacacggcaatggagactgtggtgaaaagagttaattcccggagtgactggcgaattccaatcaatgatcggaagtatgccaaatcgtgatcgattgtgatgccaaactgtgatcggagatggatgccaaatcatgatcgattcgaatgccaaactgcgatcgattcagatgccattttgcagcctgagtttaacggacactccttcctgaaatagaatggcatcagaaggagccaataatgagcagaaaaacccaacgttactctacggagtttaaagccgaagccgtcaaaacggttcctgaaaatcaactttcgatcagtgaaggcgcttcccgattatctgttcctgaaggcactttaggacaatgggttaccgccgcaagaaaagggctcaatacaccaggctcccgcacagtggctgagctggaatctgaagttatgcaactgcgtaaggctttaaatgaagcacgacttgagcgagatatattaaaaaaagcaacagcgtattttgcccaggagtcgctgaaaaatacgcgttaatcgaacaatggcgacaacaatttcccattgaagcgatgtgtcaggtatttggtgtatccaggagcggttattacgaccgggtacagcatgccccctcagacagaaaacaaagtgatgagcggctaaaactggagattaaggtggcacatatccgcactcgcgaaacatatggaacccggcggctccagacggagctggcagataacggaattatcgtcggtcgtgacagactggcacgtcttcgtaaggagctgaggctgcactgtaagcagaaacgcaagttcagggcgaccacaagctctgaccacaatctgccggttacgccgaatctgctgaatcagaactttacgcccacagcacctaatcaggtctgggtcgcagatataacgtatgttgccacccgggaaggatggctgtatctcgctggcgtcaaagatgtttatacgtgcgaaattgtcggttacgccatgggagaacgcatgacgaaagagttgacgggtaaagccctgtttatggcgctcaggagccagcacccacctgccgggctaatccaccactccgatcgaggttcacagtactgcgcatacgattaccgggtcatacaggagcagtctggtctgaaaacatcaatgtcgcgtaaaggtaactgttacgacaacgctccgatggaaatcttctggggaacgctgaaaaatgaaagcctgagccactatcgttttaagagccgggatatttcgtcagcatacgggaaaactgattgaggtcggcctcagttttgaggcctttagccagttcagccgcaagggctttaagcttcttttcgtccataatttacctgtctccgttgttggagtaaacatatcaaaaacaggcaattacacaatttaaattacagtctcaaaacaaacccaacggtcaatatagtttaccagcaagcttactagcaaagtgctaatcatttggtatctttcgtgctattctccttttacgtaagtacttaaggggaacgtatttactacttttaaagaaagctccgttactatctgaaatgcaatgagataataaacgattactctcggttagtatctcatgatcatttacagactttgcataatcgcgaatactatataacagagttttaacattacaggatgaatagtctctcttatcaactcctaataaaagaaggattatggtcagtcgtttttgctgtcttatcatatattatttagtgcagttcctaacaaaaacataacaattttctacaattgatcctgtgactacataaatatcttcaacaaaaatatttatatcagcaaaagagttattatgaatgattataaatcatcaggtgttttttcaacgaccaactccagagcacaacgataaatgtcttttctaaccaggtcacattcagatgaaatctttctgagtaaaacatttattatatccttattgctaaggaaagatattgggttcaatacctcattagcaatggagttaatgatggaagattctatttcaaattcagactgatcaatacctattttattacagaaattaactcccatataacacatctcctttacttattaatgagtgttttcaacctgctcaagccaaacaataagacgtaggacttcatccaagtgttcaaaatcaacaaaactatattttgtatgtgttttatatagttttctagctaatttcacatcacgcacagttggtataccaacttcatttgcatattttctgacagccaaggcacactggttagtttcaatgagagaaataaaaggtgcaggcgctatttctggattaaaataaataccaattgcaatatgagtcgggttcattaccactaattttgaattacgtatatcagatttagtctgctctgaaagaatctcgatatgcaactcacgccttctcgactttgtctcaaaatgtccctcttgctctatatattctctttttatctcctgtttatccatcatcatatctttcatgtataaaatgaactcaatcacaaagtcaagaataataacaaaaatagaaaatgccaagaaaaataatattatatccttaaacagccccccccaaataagatataagccatcaacactagaaaacacctgagaaaaaattatttttcggtcattaatccaaaagaaataggttgttaatgctagaataataagaagcagaatgcttttgaaaaattcttttattgtctttatactaaatattttttttaaccctttaacagggtttaatactgaaaaatcaatcttgatagctttagtcgcaagaacaaactttgtttgaaccaatgttggcaacacagccgaaagaacacagaaaaaaagtgggaagccaattatcttaaaaaagacaataaccactgcaaaaaagtatttaccctcattaatttcgaagtcattaattattacatatctgtataagagcattacatcacttaaggaaaagaatgatattatagtaaatgtccctaccagaataataacaacagtcgttaaatccttaaatttaaatgactgtcctttttttgcggcatcctttagttttttaggtgtcggcttttctgttttatttgccataatattttatctaacaaatagatttgtgaaaaatttatgttcaccgaggaaaaattgaactttagaaaagtaaatagtagaagaacagatgaaaataataaatattgcaagtaaacttttaatagttaatgatacggaaaaagcattcatctgcggagcaaatctcgataatacaccaagtaatacttctgatagtaacaatactatcataacaggactggctaaaataacagcctgacttgccaataaagtcagaaatgttaagatatttgagacacggaaagaacattgagaaaataacgggcatatattataagacaattgtatggattctaaaataaagaccataccaccactgtatagaaatacaactgcagaaaaaagattgaaaaattttgccaactcagacgtatcaacaccattggcaggatcaattgaactactaagcgttgccccacgctggttgtcaataatgctaccaacagcatgaaatatccaaaagggaagagacaagcaaaaagagagaaggaggccaacaattatttccttgaacataaggaaataaacatgttcaaaaacagaagaacctatgtctaccttaccagaagtaattaatcctgatgcaacaagaaaaatcacaggtattctaatagatggagaaattataccgttatttaaaaatggaagaaagaaaaataatggagccaatctaaaaaaaacgccgttcaggagtattaaaaacacatgaatactttcgaaccagcttgaaatgtccatcaaaccccactcttaattaaaaacattatttcatgacaaaaagacaataaaacctcaccataccatccagaaagaagtagcaaagatattgagacacctataagctttataccaaaaggaagtgtctgctcttggagttgagtcactgtctgtaacaaaccaatagataatccaataaccgtagctatacctactggccataaggaaaagataaggattaaataaagagccttattacccatataaactatatcagacacattacctccatcatctaagcaggaacattgatatattgttcaatcaaggccttagacaggataccccacccatcaagcgcaacaaataatacaagttttattggtactgatattgtgattggactcatcatcatcataccaagtgcaagtaaaatgcttgaaataacaagatcaacaacaacaaatggcaaatataaataaaaacctattttgaacgcatctttgatttcacttagcgcatatgcaggcaagagggaaaacaatgaataatcattattctctgcactctttaaatcagcattttcttcttcagacctttggaaaaaacgtgctaactcaagatctgtatgctttttcaagtattgcttatattccatcaagccagaatcagaaaaacggacaatatcagatattgtatcaaatttttgagggccatttagataattttcataaccagcttcaataatgggtttcatcacgaacaaagccataatcaatgcaataccattgagagtcatatttgatggaacctgctgcaaaccaagtgcatttcgtaccattacaaaaacaatagaaaactttatataacaggtaccagcagcaactaaaaatggtaatagcgtaaaaaaagaaagtgtagcgatgagggacatgtcactcagcatgagattactcctttaccatccaagaactaatctcgataccataaccatcctcaatagaaacaagttccccatgcccaaccaaagaaccatttactttaatatttacatgtttaactatgtcatcgggaaacttgaataattcgttttctacatacatttttagttcattgattgtcatatttttttctaaaagaataaagtccacttttacattgatatcatcataattgaataatgccaaagaaacttctgtgtgttctgactcaccattactttctgacagattaatttttgcctcattattatcattcacaatataatccccaataataacttgattacacaataataaattataaagtttctgaactatgattatatcaccagtgcgaattctttttaaagtagccagccttacattagatgaacctaagcaaaactctaaaagatgatgcaataagtcaagagaaacttgattacgtaataccggcatcttgtcaaaataaataatacctaaagaagaggaaaaacagcataaatgactcccagtagttgcatactggatttcattcgtgatccggaaagtaatcttatcagacagttctggaatcgaaaatacagatgacgactctataaagacacattttgataaatgcagtaaagattcttgtgtagaaaatcctgaaaatacctcgtatttttttttcaaaaaagataatgtatcaatacgaattacccaatccccacacacggaagtaagagtgactaaactttcactgcctgtaaatttatttttgaatgtctgaatcgaaaaacgctcgcagagttgctttgcatataaaatctgtagtttttcgttagcgtcaaaatgtttaattcttagcattcttcttcactctgttcattatatcctgtactattatttttattccccttattatgttgactgctatcaaatctataattaccctcagcatcttgtttcaaaattaagtcaagtttatttcctacaaattgatctgacggttttagaacaaaagaaccagactccactgaaatactaacagaatgtccagccccccactgtttaaagtggtaagtcatttcactatttttttgaaaaacatttttatcattttcaactattgatttttcttgcaatgacatggatggggttatgtctgcagtatttttattcattacaggcagctcaaatgatttagcaacacttccatttagtaaagaaatgatgtcagcaatattcttatcaatagacaagctatgattttttaaaccaatttgaccatttctattatcagattctagcagattatcagggtttacagaagaagtatgacttctttcgaagttcttgtctgcatttccactcttattatatgaaaagatccccctcatatcgccgatatttattttttcccttgcttttatatcttgatctggtatcaaaatattttctttaatggccattgattctggaatatcagaatatgtcgctcgttcgtttctaagttcgaacgcatttttaatgccactataaatttctcctttatcatgcttatgttcaaagtgcttatcaatagtaaaatagttagatgcaattaaagagtactcaggaatttcaaccttcctcttcttcttcaaaacgagactatcaatattatctattgaagatagtttctcacatttttctatctgtttgtcactactaaaatttaggtttatattatctaatgccatacttcatatcctcttgtgataactcatcaagtatcaactgtttggcaaagttatttcttttaatcaagccaatccgtttacatattttactctctttatgaagcagttttcttttagaagcatttgcctcatcaatatctttttgaatttcttcaattcttccctttagaagaattcgttcagactgatattggataatatgcttttttactatagcgatcttacgtcttgtattgaaaagctgactaacactctgagcgcctacaggttctaagtcattctccagttgcaggaagtatataatccttttatctaatgcctccacatctcgatacaaagagttttttttttagtgttaatatgtgtgcttcaattaatttaacagaccgttgatatttatctttgatttttaaaaccttatctaattgtttcaccaataagctccattgtctgctcataagtaaaaccgagtcgataatcttgcttcagaaaactctcaacaacagatattttgttgtagattttatcctgagaggcattctcccccggtttgtattcaccaaagtcaataattgtccttagctcttctatttcagaaagtagctcccgaaatgcagcggccataatacgatgtttttcatcaacaacctgtgtaaataccctgcttatactttttaaggaatcaattgcaggaaattgtcctttttgggctagatttctgctcaaatatatatgtccatctaaaatggatcttacctcttcagctaatggatcagcaaaatcatcatcctccaaaagaacagtgtaaaatgcagtaatagagccacctgcctttaactttcctggcctttcaagaagtctgggtaagctatcaaaaaccgaaaccggatagcctcttctggcaggtgattctccagcggctaaggccacatcacgtaatgctctggcataccttgttaatgaatcaataaaaagcgctactttatgtccttcagtcctaaaaaattcggctatagcagtggctatatatgcagcattacaacgatcaaccgaagagtaatccgaagttgcatatactaaaacacacctgttttttttctcagagtttttcaaataatcaaccgtttctgtaacctctcgacctcgctcaccaattaacccaataacatatatatcagcaccactatgttcaatgagcatattcatgagaaaagttttgccacaaccagctgacgcaaaaatccccattcgctgtccttcaccacacgtgagtaaagaatcaataaccttaatacctgttaaaaaaggcttctcaattgcagccctttcactatatagcggaggagcattatctacaggtcgataaagaatttcactgttatccgtaacagcaaacttatcagtaacctcccctaaaggattgactactgcacccaataatccacggccgacttgcgtatgaagaaactgggcagtgggcttaatcaacgtttgccgtgaaagtccacgagaatttccaatcaagcttaatattgttttttcatcatgaaatcctacaacctgagctcttgcaacaatttcattactttcaattccagcctgaatgttacaaatctcaccaatcgaaacatcgctaagacttgtttccaagattggccccgagattctattaggaaaagataattgagtgagcaattttgtatagctcattgattataagacctcatttaagatttccatcctctgataaaactcatgcagaatctctgcaaaaacaatcccatcgtgaacgtagtcatctttaataacaactcttaactgtaagtactcatcagaacgatgaagctctaccagctcattaatgctataactaaagttcattaacattaaatttaatatgttataagcagatgactgaagttttacatcacttggtgcatcaaaattagcccataacataacctgctcattgacaagagctatattaatagcaggcatactatccaaagaaatggtaatcgctgaatggctgtctaaatctgtaataattgatggaggacaaccaatcgtgaaaagactatctctaactaattgaactaaattaatgttactcatatttaaacctcactaaatagtctttaatacattaatggtatatgcttcatcaatctcagcatacgatataacgagtatactcttaaatctgttatctataagtcttttaacaaacctacgtatatctactgataccaaaagaacgaaatttttttttgcatttctcaattctctcaaagcatgtgctaaagtttccattacctcatccgaaacctctatatccatattcaagaaagagccaccagaggtttgccttatcccttttcttattgcatcctcaatatatccggaaagcatcacaactttaatctcaccagaaacagctattttactacaaatatacctagaaagtgatgcacggacatgttcaactaatgttatgacatctttttctctgggagcccaaagcgccaaagactccataataagttttaaattgcgaacagaaatattttctccaagcaatctttgtaatacctcagaaattctctgtatagtcacatgtcggaagacttcctttaatagatcaggataccgattttcaaactgatctaacatattttttgtttcttgtataccaaaaatctcatttatgttgtttaataaagcttgtgacaactggtgataaaattcatcttgagcacttttaattttagcatcaatattagttagattttctgtatatgaaacgtctacccaagaaataacacgttcattatatgaagttgaaacaacaggaatacctagagcatctatatctccattttcatctgtaatacacactttgtcaaaatatatattaaaactatcggcacgcacctcatttattaacaaaacaatatcatcgaccttaagctcattgcttgttctatataaaatagttggaagcctgacaccataatcaataaagaactgacttcttatcctttcaataagaccttccatatcatttgcatttatcttattttcggcaaataataaaattaaaggaacggtttctgaactaatagcatccagattttctatcattgccaaagatgagtcatggctattatcaatatcaaatgtaccagtatagccactagaatcagactcagacagacttttttctttttctacgacctttttataatagaataaagccgtcaaagtaactgctatcaggaaaaaaacaaaaaacggaaaacctggcaacatccctatcgccaaggcaagagctgatgtaacaatgagaacaaaaggattcccaaatatctgggacattatgttacgtcccatattatcgctatcaccattaactcttgtaacgataaatcctgcacttatagaaattaacagagcaggaatctgagagaccaatccgtcaccaatagttagtatggtataagtagataatgcaccagatagggacattccatgttgactcataccaacagaaatacctcctattaaattgacaaaaataataatgatgccagcaattgcatcacctttaatgaacttcattgctccatcaaatgaaccatacaattgactctcacgttccagaatactgcgtctttctttagctcctgcggcatcaataattccagcctttaaatccgcatcaatgctcatttgtttccccggcataccgtcaagagaaaagcgagcagctacctccgcaaccctttcagatccttttgttataacaatgaactgcacaacagttactattgaaaaaataacaaaacctacagccagactatcaccaatgacaaactggccaaatgtagtaataattttacccgcatccgcatcaactaaaattagtcgactagtacttattgataaagcgagacgaaataatgtcgttatcaacaaaaccgaagggaatgtagaaaaactcaatattctttcaatataaaatgatcccataaaaacgagaatagcaagtacaatattaagccctattaaaaaatcaacaaggtatgtaggtaatggtatgatcaacatagcaatgatcataaccataagcactaatataattaattcaggcttagtacttacttgcttaagaaaagactggatcactttcatctcctgttatctagaaagctctttcttgtatgcacttgtgacaagctctcgtagaatattgatcgcaatttctctttcactttcacttttaaatatatcatgaggactttcattaacagaagatagatatgtttgatacactacagaatcctcatgactggctcgtgatacttctataacagaagtaagaatttgctcactttcacttggatattgaaaaatatataataaagttagcaatagctgctctgcacttaaactgccatcttttagcaagcttgaggattccagtttcttcataaaaattacgtctgaagttctaatcatcttaattgcaattaatttagataacacaaaaccaaactcataagcattacagctcggtttctcagaatccatgtctacaatcagtgactgctctacaaaattcacaattgtatgtctgtgattaaaaccaaaattaccaagccattctatatactgatctgttgttgatatgttccccatgataaaaccacgataacatgcacgcaaaagctgtggttcaagtttcattttgttgccaaataatttagcctgtatagctgaatttattccagcattaacacttttttcactacctgatattatctcattaataattttagttaatgattcgacttgttcagcagtaagatctttttcattcaaaagttcccttaatgctaagacttgatcacttggatctttaaaatatctctttagtctgtctataaaatcccgatctagaggaaggttatcttttaacgttctctttaaatcaaaaatcttgtgattaatttcatcttcttccccatctaaaatacgttcctgactatcactatttgttcctttcagtttctcaaggtctcttctgtttataaatgacgaaagagcggaagccatctcatcagatgagtcaatatacttgctattagccaagccagaatcaagctctgcatcagcagtctcatctccatcatctgaatttgtcattgcattcaacttatcaatgaatgcagagctaaaaactcctgtatttttaacatcaagcatacatcacctactttatagttagtaatttaagtatgaaaccaatgatttttcgtcttccagcaaagtagtttcagatgggattatttgtgtagtcttttgtatttctctttcagatatgaggcttttatattctgcagtattataataacttgactcctttatctcacggggttgaatcagaaaaacccttacaatattactgatattacttgttttatacttgaacacattaccgattacaggaatagaggagagaaaaggaatagatactatttcgttagagttcgtttcatgcgtataaccacctatcaacaaacttttgccttgtggaactctcgctattgtactgattttggtccttccaacttctggaaggacagatgtattttcattattataattgtaatttgactgactatttcctgttccatcctctatcgttaaagacatctcaatttgtccccgagatgaaaagcgaggtataacatttatcaatgttccatacgtaacatgctccaacgaagaattgcgctcaccaactaatgacacataaaaagtcctgttattatcaaatatagccggaatattctcttgtgtcaaaatcaccggccttgaaactacatttgctttctttttctgatttaatgccataactgaagcaataaacttattgccgtccaaagtgcttatcgatgctgatgagctcatattaaatgacgcaccaaacgaatcaccaaatgaagccgttccttgccaattaacaccaaggttatttaattcagatttatcaatatcgattatccataatgataattctatatgtcgtttggctacatctaactgagtgataatatcgcgaataatctgtatttgctgatcattacctttgactaatatagaattggtttctggatatgcaataagtgatacatcctctaatatagaactattggtaactgaactaaatgaaaagtcattactatcctcagatactttctgtgtaatattaaagggtggcatcggatcattttgggcttgtctgttactcagagccttaccattatttaataaacgttctacaacagtagccacccctgggatgacaatatcctcccctctcatattataagttctgtcactaacaaatgtattttttaattttataaccccaaaattaactttatctgtcccaatagaagaaacctgcttatcaagcaaagttgctgtattagctaccagttcaaccaatgccggaggaccagatatatagaatgttttatcactaatatttcctcttataggatatctgtgatcataaaggttagcatccttcagatactgaataagataattaagtgatatattctccagcaaaataactttgcttattaattctccagagtcatatatgtataatgcatttccatctttataccatatgagtcccacaagtaatgtcagtttttcaagcatttcttctggattagataaatcaaactccccagatattctcttttttgcagcttgtttgctaacaacaataggataatttaataacgcagaaaaacgttcaaaaaaagatcctaccgtatcactttgagcaacatatttcgctatgtcattttgttctaataaatgagagtcaatattattagcattaactatcaatggtaacaatacaatcaagagagtcaaagatttaatattaaattttttcatttatttttttcactaaaaaagtaatattcgataattctcttgcagaaaagcctagtcgtgtttttatctcatttgaaaaatgggatgttgatgcatacccattattcatggcagcagaagtgattgtttgattgtgcaaaaatacatctaataaaccatttactaaccgccaagtgttcagttgttctttgacttttgcccctaaagcttttctacataaagatctaaaataagcctccgacacaccataatgctctgtcagactctttattcttacctccttttctttctcaatagttctcagaagataaagaaccaaatgatatgactcagtcattcgtatcatagccagtgcattacttacattctggtcagatgaaaagatgaatcttaacattaacttatcaagcatcgattcggtattacaactatcccgaagatcattctcatgatagaaatgaatacatttttcagcatggctatgctctactagagcaccactgaaatagtcccaaaaagcaagaaattttgcacaactattaatgttaaagcgataatgatagttcacattatcactgaattttatattttgatttcggacaaaaaaaaacccttccatgctggagccagctcgattatgttacgttctttatcttttacaagctcaacgttaactggttcatgtccactagagtaaatgtaaaacatattacttgtatttagacctttatatttactcacaatacaattatatccctcgaattaattttaatttaacttccccatccttctctataaggatggtatcatttatcttattaccttttaaaacaaccccgtctttcagcatagaaaatagtttatctatagctatcttttgtgcatccatgaagcacccatctacttccttaacattaaagttgcccatgaatttacctgcattaaaactataaactgccttaccactaatacatttagagtaaacatttatacttttattggttgtattaaacgaccataataaatcatttggaatagaaaaataatccttacttacaggaacaatatgccattctttttcagaattagatgatgactttaaagcacacccactcagtgttaatagcaatatacttaaaataaatattttcaacttattactaccatgtcgaatcatctgcctctctccctgcttcaaagacactaattaatgaatcgaaggttgtagtcttaatatagaaatcaatattgttatatatggacaatgatgcataaaatccctcatgcaaatcgacatattgatttgcatctatgagtttattaagaagtgaacttttttgtgcagctgtaagataaggaaaacgtgaattgaataattgttcaatcttattgtgcagttcatcattatctatggaatttaattcactctgtgagaaatattgcattatttcatagattatatcctcatcaaatgattgcgaagatgataatctctcatttaaattaacaaattcttcactatttaatctttgctgcagagcattgcttgcatttatttgattaatcattaacaggattctcttttaacaagtcttctgcaatttgtctcagtttcttatgtgctaactctttgaaatattttttatcattttttgttaatttttccgcaagttctattttaacacctgatattacatcctgaggactaaactccactacttgattaccactacagaatatataattattcccttcagaatattttaaaataatcttgacatctgagcgatatttatggagatcaagttctaactttttcctcagtgctaaattacacttgggcaaaaccacttttaattctgttactgtattacgcaacgaactaagccactgctccaataaagtgctaatgatcctctcatcatgtaaattttctgaaagcaaactagttatatagtcctcaatatttcttctgttttcattttgctttaacttccattcacaaatatagtcaataatatgctcaaaagcagtctgtataccaatttgataaccatcacatacagcatctatacgcagtgattcagcttttttcgtagcatcctttataacacaaattgctttcttcttactctcagtgtctctatcttttatggttttacataattccttttcaattctctttataacaacaccatcataggcatgatgtctggataccggtaaagtgcctttttgcatattgcataccttcatttaatcgggacaaatttctttctacaggcgtataatttccctccaaagcaattggattaaataataagtttattctttcatatataccaacagatatattttccttccacgcaataagatgccaaaaaccacttcgaatagcggcctccccttgcttactgattttgcccccataataactaataaccatctcactatgattaattaaccgagactcttgactaccgataaaataagcaacagctggaagtaaattccagttggcaacaaacatagcggttaccccgcttaaaggttcaatatcgcaatttagatcatatttattaattatgagattattaatctcacttctgattacgccattttctaacagttcagaaggcaaattcaaccgatttctatttatatagaacgctggatcaaaaattattgaaagatattttttataaattccatccattcttatcatattttgtttctcttgaaccaccctgttttgaatgcccataccaaaagaatgacaactagtacagccatcccgagaaataaatatattacttcatttgttaaaaattccgatttaatttccttaacaggttgtacatttgtataaacatattcttctttcggagttaatatgacagatatattttcatacttaacatcagaaaaggtgtttttcaaaaatcgcttaatattactaactaatagttcagactctttcggtgagtcatatatagcgattactgatatatgcatcggttttgaagatatatttttttcttcaagatcatagctaacatgtatttttgccgaaataacaccaccaatagaaactaaagactgttccagccgttgctcaatagcactatataaacgggccttttcagctcttggagaagacactaatgaatctgtaggaaacatttgtgagatatcaactctctccggatttggcaaatcgtacatgcgcatcaaatcaactgccgatgcaaatgtccccttttcgacttgtaccgagatcccctgtttacctccatcaacttttctagcagtaatattatggcgttctagcacagatattatttcatttgcctgtctttgagataaattagaaattaactcttcacgttgctcacatccaatcaacatcagcaacaagaataaaataaaacctttatacctaatcatataaattaagactttaataaagtttcaacagccgaaactgtttttcttgctaacgtgccagctaatgatactcctattgaataatttgagagcgtcgtctgtaacttggccaaagattctgggtttaatgattctggattgcttaccatctccattatttgtgatacttgaatatctgtatccatcttaatatcactatatttagccgacacgacgtcttccagacttgatatctcttgagattgaaaatcactggctttgataatatcaacctgattgactggataaatgtaattcatgaatgctccctattatctgaagttttgaataattgcagcatcaacatccttaatcactttcactgtattggattgcgcgttcctatataatgtatattcagataatttactttggtattcagccagcaactgtggattcgaaggatttttagctaatttatctagtgccaatgttagttcaccttgtaatgtttgagttccatcatcaaaagtttcagataatgaactcagtgtccaatcatcattcggtactgtaacactcattttatcctcacttatttttatctaaaaaaaaccagtgtttatcattcaacattacataactgtctttgctgttaagatatgatttacctttaaaatcatcatctatcaaaagtacagaaaactcaatatactgatctccatatatatttttatgttcagaaatcagtctattaatgagctcaagcttttcatctgtcagttcttctctgacagaatatgtaactttattattttcacaaatctctttatactggacatttacctttgtgaaaatacctttagcatccccccttgcattatgatcgctaagatagttgaatttaatatttttgatatacggaaattcaacaatcagagcatttttcaatttatcaagttcggtatcttttgaactatttctttctttacttaaaaacaccagtaattccgctttatcactcactaaattcaacacatacaaattgataaatgggaatcgttgattgatatattgttgaattctatttatttctttgtttgacaccagaatatatctgcatttggtaaatccggttttatttaaagctacagatgcccataccgcatcattttgtgtttcggcaagaatataattgcactgtccagtgttgactattccatatctcttatcatctataatttcagcaatttcttttacttcatctttctttaaagagagggaaaaaattataattaacacaacaataacagcaaagaaaaaaaacaccgagtggttcttatacattccgtttagtatatggcctctcgatttatcctccctcatatttttaaggtgaaaagaaatcccgtcatattcaaatgaattaaattgtgaattgatatcaatttgctcacctttttcatcactaaattgacagatattatctttgcttatggtcaaataaattataccatccgatttactatctgttacaggaataatattttctgggaaattgtcgtttccaaactcaagtgtttctatcgtccgtccgagaacaattaagttattcccataatataacggaaactcatccccgacaccgtttgtgagtttcactaataacctgaatggtgcaagatttgagttctttgcctcagacataaatcctcttatatccttcgattattctgcttgctctctgctgctatccgcaaatagcgtctatagatatcctctgcgtactttaatctttcttttttctttttcggagacgttcctgcattataggctccaacagcctcccaccctcttccatacatcatcataaactcatttaataactttgctgcataatgtacagaaagacatggatgactaattagcatttcctcagaatatcccatttctctaagtcttttagaatgaaagtcatttatttgcattataccataatctttacttccattgttattaacattgactgcaaatttattaaatccggattctttttccgcaatcgcttttaacaaacttgatggaatattgtacctttcaccagccttatcccaacaatcagctctccctaaatatgggataaaacacagcaaaataaatacgaaacgggacattaataccccttcattcttcgcgcaaattcatccttcagccaacgcacacgcgaaatataatattctgtattaacatatacatcctcgaattcatttatcccggtaagatctaatcgcgcaattagatttccgccttcatcatcagtagctaagcatatcttttctgagtaatttaggctcaacgcatatataagatcatttatattttcaggcaatgcacaaaaagggcataataaatttattgaatctccttctttttcaatatatattagcacatcatcatcaagcatgattgcttgatcatcgatatcaattaaagggatacccaaggctcggcaaataacatctgctaaatcttccatatattcctcttatacaaatgacgaataccctttcaccatattccatatttttgagtcccctattctttcagaataagatagctctaacgaagatagcggcaattttttcatgactttatttccgggcacaccagtattcatctcttggatttccatattaccactattcattagaatagtagaaaataatcttttctcttctgaggataatttactatttaattgagaaaactgacctgtttcatgttttcttattatttctcttttaatttcagcatcctgcatacctgttctgtccttcccactcttacaattccagcaaggtacagcacctatagtataagccaatagagtcattctttgtgacaatttatacggctctccattatcatttttttgtaacttcttgtttataatctcttttatctggttagccagatatatcacatcattcttacatggtggatttttttctattgcttctgcagcccaacctccaatcacaccatttttgaggaaattatcccccaacaaagaacatattgattcatcattaagcttgtcaacattcctccagcctaagcccatttttagcgctaattcatttacaccaaagttaaatgtcaccacttttagattaacgcttacttctttcagaagaccatcactattccgaattaataatttcgttggctccccccttttagagtttaacccttttaatgcgttcacctgatcttttaacatactctcctctcccccggttaaactggtcggcgtcagaagagaagtcgaaacaatctttaaatctactgttttaccagacaaagcctgtgataataactctggtctgctatataatgctgcgcttactaactcctcagctttattacgagcagcaacagctctttcagatgagttttttttcaatccataggctgatataactccatgtctgatacccgaaaaaatttccttcccctcgtcatccaccacttttgaaagccacatattcgcgatatgatcactctctctcgtagaagcacaacaaattccttttccattatactctttgacaaatatatttttattccctatcttcatatgagaggcaggtgttaattgaaaaccataatttttcccatgatgactaatattcttgttgaccggtccccaattcttattatttaaataatcagtaaatatttgttttattttcgtttgcaattcctttttatttttaatgcctagctgtgctcttatctcttcacttattactttattaagctcattaatatgtcttttattaacttcaccaagtttataggtatctaatcgttccatcatggctttaatttttgacgtatcaccacctgccaactttattgcctcgtaaaaaacttcagctctgtcacgcgcaaacaaaaggctttttcctgttaagcttgtttggttctgtaaatataatcgattaagcgcttcgatccccttatttctttctccatttttaactctttgatatgaaatcacgcttacacctttgtgctttccggtttcttctgcgcctttataggaatctccgctttgaaatgaaacctgatgcaatcccaaattagttatgtgcatttaattatcctcagtcataaatggggcattgattgcagttttgtacctcgttttacaacaaaaaagatacttattcaacagggttctctctttttctataacactccgagcctgatatatcagacactttaacttgtgggatttcatgatgaaacgagcactacatatgctcacgaggtacatattatgtacaacaaaagaaaatttgattatgttaacaggctcaagtttctaaatttattaattgaatatattatatatcaaatacattttacatccccacaatcaccacgtaatggagagttaataaagtatgatcctcaaaattagcaatttcattgacgcaagcaatacaaaagggcctatacgcgttgaagatacagagcatggacctatattggtagcacagaaattcaacctaaaggatctctttttcagaacattaagcactattaatgcaaagataaattcacagatattaaatgaacaactaaaaaattatcgactagcgaatcaaaaatccttattgcttttcctcaagacattagctagcgaaaaatctgcagaaagtgcgtttgccgcctatgaagcagtcaaaaattcaattcaacattcttttacaggtaaagacatcaaattaatgttgaataccgcagagcgttttcatggcatcggtacagccaaaaatcttgaaagacatttagtttttcgttgctggggaaatagaggcataacccatttagggcataccagtatcagtataaaaaacaatttactacaggaaccaactcatacctatctcagctggtatcccggcgggaatgttacaaaagatacagaaataaactacctttttgaaaaacgctcaggttacagtgtcgatacctataaacaagacaaactaaatatgatttcagaccaaacagccgaaagacttgatgcaggccaagaggttagaaacttattaaattcaaaacaagatcaaaataacaacaaaaaaatattttttcccagagctaatcaaaaaaaagacccctatggttattggggggtcagtgctgataaagtatacattccattatcaggtgataataaaacaaaggatggtaaaatcagctataacctttttggccttgatgaaacaaacatgtcaaaatttatatgccaaaagaaagcagatgctttcagacaattggcaaattataaattaatcagtaaatctgaaaactgtgctggtatggcacttaatgttcttaaagcaggaaattctgaaatatactttccactccctgacgttaaattggttgctacacctaacaatgtatacgcgtatgcaaataaagttaggcaacgcattgaaagtctcaatcagtcttataacgagataatgaagtatattgaatctgattttgatctttccagattgactcaattacgacgtagctatcttaaaagtttcaataaaattaaccttatccacaccccaaaaacattcaagcctttatcaatttcactttacaaacatcctactgaaaatgtatcttcagaagactttgatgcggtcatcaacgcttgccactcatacttagtaaaatcagcacccagcaatatgtctcgtgtattaaacgaattaaaaaccggagcaacagataaaaaagaagaaattatcgaaaagtcaataaaaactattgattattacaattctcttaaatcgccagatcttggaaccaaactttacattcacgacttattacaggtcaataagcttttattgaataactctcattctaatatatagaaggccatagaaatgtgtcgcaaactatatgataaactttatgaaataacaggtgcaaagcttgattttaacgataaaaatcaagcctttatattgctcgaagaacaaatacctgtttgtataacagataatgatgaatacatctttttgaccggacttcttaatgaacatgaactttttacagaaaatattattaatccagaacacatactcatccttaactactctttgagtagagattatggttcttctatctgtcttctccctgatactcatcaatgtgttttgaccaagaaacactacaaaaaatatctttcacctgatgaactcattgaatctttgtatgaatttttattttgtattaaactaaccattgcaaacatcacttcagaagtgaactaacatatagggggtatcatgcaaattctaaacaaaatacttccacaggttgaattcgcgattcctcgcccatcatttaattcgttaagttacaataagcttgtaaagaaaatattatctgtttttaacttaaaacaacgctttcctcagaagaattttggatgcccggtgaatataaataaaatcagggacaacgtcatagacaaaataaaagatagtaattcggggaatcaactattttgttggatgagccaagagcgaacctcttatgtctcttcaatgataaacagaagtatagatgaaatggccatacacaatggagttgttttgacctcagataacaaaaaaaacatatttgcagctattgaaaaaaaattcccggatataaaacttgatgaaaaatcagcacaaacatctatcagtcatacagcactaaacgagattgcctcatctggcctcagagcaaagattttaaaacgctatagcagtaacatggatttgtttaacacacaaatgaaggatcttacaaatctagtatccagttccgtctacgataaaatatttaatgaatcaacaaaagttttacaaatagaaataagcgcagaggttctaaaggccgtataccgtcaaagcaatacaaattaaatctgtttatattaaatcttatacttattaaagtaaagcagacaaaaaggagaccttatgtctttaaatatcactgaaaatgaaagcatctctactgcagtaattgatgcaattaactctggcgctacactgaaagatattaatgcaattcctgatgatatgatggatgacatttattcatatgcttatgacttttacaacaaaggaagaatagaggaagctgaagttttcttcagatttttatgtatatacgacttttacaatgtagactacattatgggactcgcagctatttatcagataaaagaacagttccaacaagcagcagacctttatgctgtcgcttttgcattaggaaaaaatgactatacaccggtattccatactggacaatgccagcttcggttgaaagcccccttgaaagccaaagagtgcttcgaactcgtaattcaacacagcaatgatgaaaaattaaaaataaaagcacaatcatacttggacgcaattcaggatatcaaggagtaattattatgcataatgtaagtaccacaaccactggtttgtctcttgccaaaatattggcttccactgagcttggagacaatactatccaggctgcaaatgatgcagctaacaaattattttctcttacaattgctgatcttactgctaacaaaaatattaatacaactaattcacactcaacttcaaatatattaatccctgaacttaaagcaccaaagtcattaaatgcaagttcccaactaacgcttttaattggaaaccttattcaaatactcggtgaaaaatctttaactgcattaacaaataaaattactgcttggaagtctcagcaacaggcaagacagcaaaaaaacctagaattctccgataaaattaatactcttctatctgaaactgaaggactaaccagagactatgaaaaacaaattaataaactaaaaaacgcagattctaaaataaaagacctagaaaataaaattaaccaaattcaaacaagattatccgaactcgacccagattcaccagaaaagaaaaaattaagccgggaagaaatacaactcactatcaaaaaagacgcagcagttaaagacaggacattgattgagcagaaaaccctgtcaattcatagcaaacttacagataaatcaatgcaactcgaaaaagaaatagactctttttctgcattttcaaacacagcatctgctgaacagctatcaacccagcagaaatcattaaccggacttgccagtgttactcaattgatggcaacctttattcaactagttggaaaaaataatgaagaatctttaaaaaatgatctggctctattccagtctctccaagaatcaagaaaaactgaaatggagagaaaatctgatgagtatgctgctgaagtacgtaaagcagaagaactcaacagagtaatgggttgtgttgggaaaatacttggggcacttttaactatcgttagtgttgttgcagcagctttttctggaggagcctctctagcactggcagctgttggtttagctcttatggttacggatgctatagtacaagcagcgaccggcaattccttcatggaacaagccctgaatccgatcatgaaagcagtcattgaacccttaatcaaactcctttcagatgcatttacaaaaatgctcgaaggcttgggcgtcgactcgaaaaaagccaaaatgattggctctattctgggggcaatcgcaggcgctcttgtcctggttgcagcagtagttctcgtagccactgttggtaaacaggcagcagcaaaacttgcagagaatattggcaaaataataggtaaaaccctcacagaccttataccaaagtttctcaagaatttttcttctcaactggacgatttaatcactaatgctgttgccagattaaataaatttcttggtgcagcgggtgatgaagtaatatccaaacaaattatttccacccatttaaaccaagcagttttattaggagaaagtgttaactctgccacacaagcgggaggaagtgtcgcttctgctgttttccagaacagcgcgtcgacaaatctagcagacctgacattatcgaaatatcaagttgaacaactgtcaaaatatatcagtgaagcaatagaaaaattcggccaattgcaggaagtaattgcagatctattagcctcaatgtccaactctcaggctaatagaactgatgttgcaaaagcaattttgcaacaaactactgcttgatacaaataaggagaatgttatggaaattcaaaacacaaaatcaacccagattttatatacagatatatccacaaaacaaactcaaagttcttccgaaacacaaaaatcacaaaattatcagcagattgcagcgcatattccacttaatgtcggtaaaaatcccgtattaacaaccacattaaatgatgatcaacttttaaagttatcagagcaggttcagcatgattcagaaatcattgctcgccttactgacaaaaagatgaaagatctttcagagatgagtcacacccttactccagagaacactctggatatttccagtctttcttctaatgctgtttctttaattattagtgtagccgttctactttctgctctccgcactgcagaaactaaattgggctctcaattgtcattgattgcgttcgatgctacaaaatcagctgcagagaacattgttcggcaaggcctggcagccctatcatcaagcattactggagcagtcacacaagtaggtataacgggtatcggtgccaaaaaaacgcattcagggattagcgaccaaaaaggagccttaagaaagaaccttgccactgctcaatctcttgaaaaagagcttgcaggttctaaattagggttaaataaacaaatagatacaaatatcacctcaccacaaactaactctagcacaaaatttttaggtaaaaataaactggcgccagataatatatccctgtcaactgaacataaaacttctcttagttctcccgatatttctttgcaggataaaattgacacccagagaagaacttacgagctcaataccctttctgcgcagcaaaaacaaaatattggccgtgcaacaatggacacatcagccgttgctggtaatatatccacatcaggagggcgttatgcatctgctcttgaagaagaagaacaactaatcagtcaggccagcagtaaacaagcagaggaagcatcccaagtatctaaagaagcatcccaagcgacaaatcaattaatacaaaaattattgaatataattgacagcatcacccaatcaaggaattcgacagccagtcagattgctggtaacattcgagcttaatatatccaagagccataataatatatggctcttcctgtaaggaaataaccatgaatataacaactctgactaatagtatttccacctcatcattcagtccaaacaataccaacggttcatcaaccgaaacagttaattctgatataaaaacaacgaccagttctcatcctgtaagttcccttactatgctcaacgacacccttcataatatcagaacaacaaatcaggcattaaagaaagagctttcacaaaaaacgttgactaaaacatcgctagaagaaatagcattacattcatctcagattagcatggatgtaaataaatccgctcaactattggatattctttccaggaacgaatatccaattaataaagacgcaagagaattattacattcagccccgaaagaagccgagcttgatggagatcaaatgatatctcatagagaactgtgggctaaaattgcaaactccatcaatgatattaatgaacagtatctgaaagtatatgaacatgccgttagttcatatactcaaatgtatcaagattttagcgctgttctttccagtcttgccggctggatctctcccggaggtaacgacggaaactccgtgaaattacaagtcaactcgcttaaaaaagcattggaagaactcaagaaaaaatatgaagataaaccgctatatccagcaactaatactgttagtcagaaagaagcagataaatggcttacagaattaggtggaacaatcggcaaggtatctaaaaaaaacgggggatatgttgtcaatataaacatgaccccaatagacaatatgttaaaaagcttaaataatctaggtggaaatggcgaggttgtgctagataatgcaaaatatcaggcatggaatgccggattctctgccgaagatgaaacaatgaaaaataatcttcaaactttagttcaaaaatacagtaatgccaatagtatttttgataatttagtaaaggttttgagtagtacaataagctcatgtacagatacagataaactttttctccatttctgaggtgcgccatgcataatgtaaataatactcaagcgccaacattcttatataaggcaacttcaccatcatcaacagaatacagcgagttaaaaagcaaaatatccgatatccatagttcgcaaacttctctaaaaacaccagcatcagtgtctgaaaaagaaagctttgcaacgtcttttaatcagaaatgtcttgattttttattttcttcctcagggaaagaagatgtgttaagaagcatttattccaactcaatgaatgcgtatgccaaaagcgagattctcgaattttcaaatgttttgtactccttagtacatcaaaatggtcttaattttgaaaacgaaaagggacttcaaaaaattgtcgcacagtattcggaactaattataaaagataaattatcccaagattctgcctttggaccatggtcggcaaagaataagaaactccatcaattacgacaaaacattgagcacagacttgcactattagcacaacaacacacatctggtgaagctttatcattgggacaaaaactcctcaatactgaagtatcatcatttatcaagaataatattcttgctgaattaaagttaagtaatgaaactgtttcatctctcaaactagatgatttagttgacgcacaggcaaaacttgcctttgatagtttgcgcaatcaacgtaaaaatactattgatagtaaaggatttggtataggtaaactgtcaagagacttaaatacagtagccgtgtttcctgagctgttgagaaaagtccttaatgatattttagaagatataaaagattcgcatcctatccaagatggcctccctacacctcccgaagatatgccagatggcggaccaacccccggagccaatgagaaaacatcccaacctgtaattcactatcatataaataatgataatagaacttacgataatagagtttttgacaacagagtatatgacaatagctatcacgagaacccagaaaatgatgcacagtctcctacttctcagacaaacgatctattatcccgtaacggaaactcattactaaatccacaaagagcactagttcaaaaagtaacttccgttctaccacactctatatcagatactgtccagacatttgcaaataattcagctttagaaaaggttttcaaccatactccagataattcggatggaataggttcagacctgttaactacgagtagtcaagaaagatctgcaaataactctctttctcggggacacaggcctctgaacatacagaactcttcaaccaccccccctctccacccggaaggagtgacaagcagtaatgataactcatcagatacaactaaaagtagcgcttctctttctcatagagtagcttcgcaaatcaataaattcaactcaaacactgattcaaaagtacttcagactgattttttctcaagaaatggagacacatatttaacacgggaaacgatatttgaagcgtcaaaaaaagtaacaaactccctaagtaatcttatatctctcattggaactaaatcaggaacacaagaacgagagttacaggaaaaatcaaaggacattacaaaatccacaacagaacacagaataaacaacaaattaaaagttacagatgcaaatacaataaactacgtaacagaaaccaacgcagatacaattgataaaaatcatgcgatctatgaaaaggcaaaagaagtatctagcgccctcagcaaggtattgtcaaaaattgacgatacctctgcagaattacttacagatgatatatctgatttaaaaaataacaatgatattacagctgaaaacaataatatatataaagcagcaaaagatgtaaccacttccctatcaaaagtattaaagaatatcaataaggattaaaatatgataaaagaaaaaatattatcaatagtggccttttgctatggtatagcgtacagcaaactatctgaagaaactaaatttattgaagatctttctgccgattctctttctctgattgaaatgctggatatgattagttttgagttcaatctacgtatagatgaatctgcattagaacacattattactattggagatcttatttctgtagtcaaaaatagtacaaaatcaatatagtaattgataagcattttttcatctatggagctctcacatcagagctccacaagaatattattcttttatccaataaagataaattgcatcaatccagctattaaaatagtaatttttaagactaccgttgactatcatcaacagttacatacaatcttgctcacactgcatttaacttttgtcaatataaatcaatatgaataaacagggtgtgatatggtggatttgtgcaacgacttgttaagtataaaggaaggccaaaagaaagagtttacactccattctggtaataaagtttcctttatcaaagccaagattcctcataaaaggatccaagatttaaccttcgtcaaccaaaaaacgaatgtacgcgatcaagaatccctaacagaagaatcattagccgatatcataaaaactataaagctacaacaattcttccctgtaataggaagggagattgatggtagaattgaaattctggatggcactcgtagaagagcatctgcaatatatgcaggagcagatcttgaagttctatattcaaaagaatatatatctactcttgatgccagaaaactagcaaacgatatacaaacagcaaaagagcatagcatccgagaacttggtatcggtcttaattttctgaaagtatcagggatgtcctataaagacatagccaaaaaagagaatctgtctcgcgcgaaagtcactcgtgcctttcaggcagcaagcgttccacaggaaataatatctctatttccaatcgcgtcagaacttaactttaatgactacaagatattattcaattattataaaggacttgaaaaggctaatgaatctcttagttctacactaccaatattaaaggaagaaataaaagaccttgatacaaatttgcccccggacatatataaaaaagaaattttaaacatcataaagaaaagcaaaaacagaaagcaaaacccttcgctaaaagttgactcgttatttatttctaaagacaaacggacttacataaaaagaaaagagaataaaacaaacagaactctaatatttacattatctaaaataaacaaaacagttcagagagaaatagatgaagctattcgggatataatttctcgccatctatcgtcttcataaacatcgaacactgatgtaaactgcccaattcagataataaaaatcttcgttgaacttgactttcagccggtggggtaattccgtcggctatctgttattcaatccccacgcactgtgtttccttaaaattacggtgaaaactcataccctgctgaccacgagaacattatctggtgaaattagtaatattcatatcattgaaaatgaattgtcaactaagggatgcggcatgatggtcaaaattttatgtgaagatgagtgactggccgaaaatattcagcgtatttttaattgtttcaataaaaacagcgaggattagcagtcaacgcttttactactcaatttttttacatggtgataaacaaaaaatccaccattaacagacttcgttcacagtatcaaaaaagctttaagaatatattagcagatttctcaaatataattactgttttattctcctaatcttcccataaaatgaccgaattacattattttataattataaacaagtttgattttaaaatcatattaattattaacaagagatttatttttctaaccccaaaagttatgcttagtcaaagagtaacttataatatccatacccctcatagggcttacagtttaaattgcgggtataaaatacatctattttcaaataaaataaagcatactccaattacaacgagtgactaaaataaaaatatcatccaagtcaatcactgtttgctctgcaaaatatgtcagccttctcaaacttaccagcacttaaatatttaagcacacaatcattccttgagggagttcacttatttcaagagaaacatttcgtcttcccacacaactaatccgcccaatatagctaaatttgtactcagtaatatcatttgttatgccccccgcccccaaaatattcaagctccttcacagataaaaaacgacaatttacaaagcctcattagttataactatggaaatacctattttagtatatcctataaagttagaattacttcttcttgcacctaattccatagttgaaaaacaagaataattttctcctacagctggatccatatagcttccatcaggtctacacaacacccagtggaggccgacaggaactccaataaaggatacagcaacagcttccaaaactctttggttagaagataacacatctcgttgatgtactatattacatcccatgccaattaataagtcccttgcatcgggataaataaaactaattacttgcgaaaaaatgttactttcttcaacgacataagcattcaatccaagaagcctcgtagccatgactatcttatcaggcatagaataaccggcatccgcgatattatctttgtgtattctcggattataattaccactagttattaagtataaatcccgctcacaacgattatccaaatcaagagaactcttccttgtcatttcggacatagaccctttgtattgtggtattttatcaacccctaattcctttgcagcacagagtaaactggcagctccacaagaattgtcatacatctgcttcctcgccaaaaaacaaccttgttcgtttactctattctcatttaccctcacatcagtctgatgagatatcatatattctcttggtatagccccctgcaataaaaccccatataacataacacctgtatgaatacatccccgtttgcaaggcttccgttgttccgacatcgacgagtaattgcagcacagtattgcatccagcaatgcgaactactatgagaattagctgaccgggtccttattccccgaacgaactttaaatccaaaacctcatactcttttccgtcgtcatcacgtccgtcaccataacaacctttcagcatccacccttttattaagagttaatgtccatcagcatcagcaattaataccgcctgatactgacatatttttgtcgtatttacgtccataatcggttattgtgattcaggtcacaccagcttatttgctattgttacaataaccaccttggttgagagctgttttttcaactttgttatcaactcccccaactccttggtcagtaaggctgccgaccgagcacagtgaatagatagtatccgcaggtaccaccatgcttacttaatactcagaaggcgaagtgctctccaattaactctgccgcactatgcttataagctgcttttcaatgccgtctatcaacatgtccagttcattcaatctgttgtactggtaatccaacgcagtaatgatatatgcaggtattctttcatccactccagtaacgtttcatgcaaagcattaatttacgcgctacggaacttcaccagttgactgagcaaacgatgtgggaccagtactgcctgcagttcttctattgttacagcgatttctttaccaaacagctgaaccaccatcaagataaagatagcctgaacattcatcgcaccattttgttaccaatggcaaaagatttaacgaaccgtgcctgcatcaggcggactttttatccctgctttgttagctccctaaccttgtacagagagcctccataagctcccatatcaagcagacatggttcgcagttactgacatacaccagaaaactctgacagcagactcacttatcctccacttcactcatatccttattggtgagatgaacctgaatgacatgttttgcgacataaacgccaaccagtgtatatttcacctgtagagattctacaatctgtagcgttatgcatctaatgttaggctctatgatgtcagaaatctgcgagactctacaccttctgcatttacctttatttatcaccatgctcctcagttaacggagaacgtgttcattgcattttgtcacttacatttcgcctttggtaccggggggatataggttccaacgttccgcatgagaacctgaaccggcttcacgccaccttgataccagatgccgcacaggcagtcaacaagtgtcacgaacggtgcaatagtgatccacacccaacgcctgaaatcagatccagggggtaatctgctctcctgattcaggagagcttatggtcacttttgagacagttatggaaattaaaatcctgcacaagcagggaatgagtagccgggcgattgccagagaactggggctctcccgcaataccgttaaacgttatttgcaggcaaaatctgagccgccaaaatatacgccgcgacctgctgttgcttcactcctggatgaataccgggattatattcgtcaacgcatcgccgatgctcatccttacaaaatcccggcaacggtaatcgctcgcgagatcagagaccagggatatcgtggcggaatgaccattctcagggcattcattcgttctctctcggttcctcaggagcaggagcctgccgttcggttcgaaactgaacccggacgacagatgcaggttgactggggcactatgcgtaatggtcgctcaccgcttcacgtgttcgttgctgttcccggatacagccgaatgttgtacatcgaattcactgacaatatgcgttatgacacgctggagacctgccatcgtaatgcgttccgcttctttggtggtgtgccgcgcgaagtgttgtatgacaatatgaaaactgtggttctgcaacgtgacgcatatcagaccggtcagcaccggttccatccttcgctgtggcagttcggcaaggagatgggcttctctccccgactgtgtcgccccttcagggcacagactaaaggtaaggtggaacggatggtgcagtacacccgtaacagtttttacatcccactaatgactcgcctgcgcccgatggggagcactgtcgatgttgaaacagccaaccgccacggtctgcgctggctgcacgatgtcgctaaccaacgaaagcatgaaacaatccaggcccgtccctgtgatcgctggctcgaagagcagcagtccatgctggcactgcctccggagaaaaaagagtatgacgtgcatcctggtgaaaatctggtgagcttcgacaaccccgttactttattcgtaccccttataatggggtgttagccagcctgaggtgtactggcaatagcggacaccaccatttgttctttttctaagcagtcatctgataatattttatcctgaaggctgccggggagatattccccagacgagagtgacgacgctgacgattgtagaaaatctcaatgtattcccgtattactgagatggcctcatcccggctcttaaaacgatagtggctcaggctttcatttttcagcgttccccagaagctttccatcggagcgttgtcgtaacagttacctttacgcgacattgatgttttcagaccagactgctcctgtatgacccggtaatcgtatgcgcagtactgtgaacctcgatcggtgtggtggattagcccggcaggtgggcgctggctcctgagcgccataaacagggctttacccgtcaactctttcgtcatgcgctctcccatggcgtaaccgacaatttcgcacgtataaacatctttgacgccggcgagatacagccatccttcccgggtggcaacatacgttatatctgcgacccagacctgattaggtgctgtgggcgtaaagttctgattcagcagattcggcgtaaccggcagattgtggtcagagtttgtggtcgccctgaacttgcgtttctgcttacagtgcagcctcagctccttacgaagacgtgccagtctgtcacgaccgacgataattccgttatctgccagctccgtctggagccgccgggttccatatgtttcgcgagtgcggatatgtgccaccttaatctccagttttagccgctcatcactttgttttctgtctgaggggacatgctgtacccggtcgtaataaccgctcctggatacaccaggtacccgacacatcgcttcaacagggaattgttgtcgccattgttcgactaacgcgtatttttcagcgactcctgggcaaaatacgctgttgctttttttaatatatctcgctcaagtcgtgcttcatttaaagccttacgcagttgcataacttcagattccagctcagccactgtgcgggagcctggtgtattgagcccttttcttgcggcggtaacccattgtcctaaagtgccttcaggaacagataatcgggaagcgccttcactgatcgaaagctgattttcaggaaccgttttgacggcttcggctttaaactccgtagagtaacgttgggtttttctgctcattattggctccttctgatgccattctatttcaggaaggagtgtccgttaaactcaggctacctcactcccgtaaagacgggagtcaagttcccgctccgacataactgcgtgcaatggattttgaaggaaccgtcagcccattccccggaggcaaacatgggctggcaaaagtgtagcggtattaggcacagctatttaggaaagagggaccatctccatctcattaaatggaccacgccagacaattcactgtcatgctgatgctgcactgtggggacagaatcactcatgatgacttcgataatcgacgacattattatttggccgagcttttaggtcatatctcaaatcgtttgcctaatattgctccattttttaataaaaaatcaatcatttttgaatctttgcttttcattgcattgtcaagcatagtgtccccactatttgctttttgaaattttttgtttacatcaaccaatccattattgataaaatattctagcactttatagttggcaccatctttactaagtaagtattcaacatcatacagattacaatatctattgttgttcattttctccatctctgcgacatcttgttttgtaaaattaaatttagatagtaaataaagtgctatatcagcttttttattgcctaaagaataagcaagggcctgatgagccatttttttatcagtcactttattgttaatctcttcataattagatgctttaatcgcatcctccaaagatatgtctctgagttttatttttttgaagttatctgtacttagcattcttggtatatgatactcaagctgaaacacaaagtttataattctatccagactaacaggatcaatatttttgttataacaaaactctctgaatccttgatctttactctttcttataaaatcaataagatgcaacccaattccaagcctcatatctttagtgttaaatattggaacatcagttttcccctgattaccatgcacatacctgctaacttcatccacaacctctttaaagttatctaagaaaaatgattggtgctcatggtaaaaaacaggtggaatagcattatcaaagtgatcggttaatgtcatatatccatatggagagtcatgatcaatgatatacgcattggcgccaaaatctacagtatgatgtgttttatttagtgggagtttttcttgatgattggaaatttcaaccccaaaaaaaacaaaatcgtggttagagatttctttaatatcctcagagtatgtattcttatgtgtttgtatattttcagactgaattttatcaaaggatttaatattaagaatattatttgacagattagagttactttgatgtctgaagttaaaattagaattaataataatagaaagtaagtgacgttcgtctgaagaaagaggatgaactttttgtattatctcagagatatagtgtctggacaccttatcgagatgctcaagtgaccgaaatgatgaccgtgctccagatggcgcttcaatagaaagatttagtgatttgaaactggtcccttgagaaaacattttgctataactttgcgctgctatagacttccttaaaaagtttttcacagtattagcacagtgatctaaatgtttttcacttatgccagaagaagatgagtttgttacgcgtaatatatttttttgtaatgcttttgttgctttattcggattcgtttttccatcaacaagatcaatattattctgaacattaatatgatttactgcttcaggtattttcatccccccccccaattatgtgcgtttttatctgtggttatttagataacattatctaactggacactttcttgtatgtattatagctaaatgaacagattttaatatgttgtatggttttcactctgaaatcccctgaatttaatcacgtttcctttactgaaggcaattttaaccgtagtatcaattattaacccttgggcttgatatccctctcctcttctgggcacgggggatgtcagattacttattacagaaaccaaaagaattgtaagcgtcatctcagtgtcgtctatttcaatgtctgatgattggcgatgatagagtcctcttataagtaagcgccccatcagcgacgtcttgtgaaaattgtcctgtctggcaacaatcgcgcccatctatattgatggacacgaacgatgaattcccagacaacaaaagatattccctgcttccgttcttatttgcctgatgccctgcgtttaagatttgaagataaactgaccatccgggccatcgctcagcgtctgggtctcagtcattccacaatacatacgctttttcagcgatttcttgcatccggtatcgcatggccattgcccgattcagtttcattcgctcaacttgacgccatcctttatgccaacagaaagaaggaattaacagagcctcaaatcagagaaggctcatggcgaaaagaacggcgaaccagctatagccgtgaatttaaggtccgtctggctaagcaggcattacagcctggtgctgttgttgcccggatcgccagagaacacgatatcaatgataacctgctgtttaaatggaaaagccagtacgaggacggcttactgagcgatgatgacatacaggaatgcatgcctgtcccggtggcactgactgatacgccggagccgaccagaccagttacaaatcccttctggcgtaagaaccacggcagcctggcggctgcaaatcggggtgtggcagaatatgagctttcagagtaaccatccagaatgagttcccatgtcacacaacctggagcaccagaaagtccacaccagaatggtgaaagaggttctgaaagccgtcgccagagccaataatcacccttatcagtccgtatttacagactttatcgccggtcatccctcctgcacggtctgtttctgggaaaccttccacaaaatgtacccggattcaccgtacgaatacgtcacgttctgccatacctgccgtcgttttgatttatacgaaacagaagcggagatgaaggcggatgacccgaagtggtggtgattttcagccccgcccgctgaaacgtctgttcacggccaaccagtgctggacatccttcctggatgcgggcggtctgcgcgatatcgaggttgaggctgtcacaaaaatgctggcctgcggcacgcggatactgggagtgaaggaatacaactgcgataaacctgagtgcccccacgtcagatacgtcactaactcatgcggcagccgtgcctgcccgtcctgcggaaaaaaggccacagacctgtggatagcgacacagctgaatcgtcttcctgactgcgactgggtacatctggtcttcaccctgccggacacgctgtggccggtgttcgaaagcaaccggtggctgctgaatgacgtgtgccgtctggcggtggagaatctgctgtatgccgcccggaaacgggggcaggaacccggtatcttctgcgccatccacacgtatggccgtcgtctcaactggcatccgcatgtacatgtgtctgtaacctgtggatgtctgaataagcatggtcagtggaaaaagctgagcttcctgaaagacgcgatgcgttcgcggtggatgtggaatatgcggcaacggcttctgaaagcgtggtcagaggggctggcgatgccggagtcgttgtcacatatcacgacggaatcacagtggagaagcctggtgctgaaagccggaggaaaatactggcatgtgtacatgtcgaaaaaaacggccggcgggaggaatacggcgcgctacctgggtcgttatctgaagaagccgccgatagcggcctcccggctggcacattacaacggaggggcgagcctgagcttccgttacctggaccacaaaacgggagaaacggcgacggaaacgctgacacagcgtgagctggtcgcgaggctgaaacagcacatcccggagaagttttttaagatggtgaggtacttcgggttccttgccaaccgggtgtgtggagagaagctgccgcaggtgtaccgtgctctggggatggataaaccggaaccagtggcgaaagtgtgctatgcacaaatggtgaaacagttcctgagtcgtgacccgttcgaatgcgtgctgtgtggcggccggatggtataccgccgggccattgcggggctgaatgtggaggggctgaagaaaaacgcgcgggatatcagtctgctgaggtatatgccggcctgacacaggctaagtgtgcccgggaatcggtgcaagtataaaaaacagcctgttgcggtataagaatcgaccatgcagagagagaaaacaccggagtggtggggaaaaacagaaaggctcacgtggcataaggcgcggacaggggtatcgcttagtttttcaatttcctatacgtcaagctgtgcacgtccatccgatgccattttctcatttgactttcgggggattactgcctttataccgttatttttcagctcgttgcgcaatgcgcgccctgaatacgctttatcagccagtaccgcatgtccacggcgtttcatgctgccgttctgacgctgaacgccaatcccgtccagaagtcgttgcgcgaactggctttcatgagcttgtccggggcttagcacgatatttaacgggaggccacttccgtctgttgccagatggattttggtgccaaaaccgccgcgagagcgacccggcccattatctccgtcgatatcgggatgtttttttgagcaccggcggcacatttcagcgcccggatattactgccatccagcgcggtggcagaccagtcaataaagccgtttgcatcaagggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgtgcagttgcccggcatccaacgccattcatggccatatcaatgattttctggtgcgtaccgggttgagaagcggtgtaagtgaactgcagttgccatgttttacggcagtgagagcagagatagcgctgatgtccggcagtgcttttgccgttacgcaccaccccgtcagtagctgaacaggagggacagctgatagaaacagaagccactggagcacctcaaaaacaccatcatacactaaatcagtaagttggcagcatcacctgcatcaagtagcgaaagcaacctgttgaaaataatattaatcacgcctgactttgaccatcggttaaagcggttataaacagttttccatgacccatatcgttcgggtaaatcgcgccatggtgcaccggaacataacacccagaacatgccattgatgattttacggtgctcagcccatgggcgtccggcccgtggtgtggcgggttcaggaggtaacaggggcttgatgatggtccatgcctcatcgggaaggtcgtagcaagccatagttcaatatgttgtgtaaacagacagttactataactcagatgattaagggacacagcctactattccgcgcagctatttagctctctttttgttgagcgcccatgattctttcatttattgttactaattttttatgttatcatcatgttatgagcatgccctgttctgcgtctctattttatctatcatattctgagcattaactacataacaaggcgcaagatatatatccaaaaacaaataatactgttgcaataattcctttcagtaaaggaagtgtgattaaactcaggggatttcagagtgaaaatcgtacaacatattaaaatatgttcgttataatacatacaagaaaatattaagttagataatattattctaaataactgcagataaaaacgcacataatgggggatgttttatgaaaatacctgaagcagtaaatcatattaatgttcagaacaatattgatcttgttgatggaaaaataaatccgaacaaagatacaaaagcattacaaaaaaacatatcatgcgtaacaaactcatcctcttctggcataagtgaaaaacatttagatcactgtgctgatactgtgaaaagctttttaaggaagtctatagcagcgcaaagttatagtaaaatgttctctcaagggaccagtttcaaatcattaaatctttctattgaagcgccatctggagcacggtcatcatttcggtcacttgagcacctcgataaggtgtccagacactatctttctgaaataatacaaaaaactcatcctctttcttcggatgaacgtcacttgctctctattattattaattctgattttaacttcagacatcaaagtaacgctaacctgtcaaataatactcttaatattaagtcctttgataaaattaagtctgagaatatacaaacatataaaaacacattctctgaggatattgaagaaatagctaaccacgattttgttttttttggggttgaaatttccaatcatcaagaaacactcccactaaataaaacacatcatactgtagattttggtgctaatgcgtatatcattgatcatgactctccatatggatatatgacattaaccgaccactttgataatgctattccacctgttttttaccatgagcaccaatcatttttcttagataactttaaagaggttgtggatgaagttagcaggtatgtgcatggtaatcagggaaaaactgatgtgccaatatttaatactaaagatatgaggcttggaattgggttgcatcttattgattttataagaaagagtaaagatcaaagattccgagagttttgttataataaaaacattgatcctgttagtctggatagaattataaactttgtgtttcagcttgagtatcatataccaagaatgctaagcacagataacttcaaaaagataagactcagagatatatctttggaggatgccattaaagcatccaattatgaagaaattaacaataaggtgactgataaaaaaatggctcatcaagctcttgcttattctctaggtaatgcaaaatctgatatggcactttatttactatctaaatttaattttacaaaacaagatgtcgcagagatggagaaaatgaacaacaatatgtattgtgagctgtatgatgttgagtatttacttagtgaagatagtgccaactataaagtgctagaatattttattaataatggattggttgatgtaaacaaaagatttcaaaaagcaaatagtggggacactatgcttgacaatgcaatgaaaagcaaagattcaaaaatgattgattttttattaaaaaatggagcggtatcaggcaaacgatttgggaggtgacctaaaatatatttaggttgtgccctgaatctgacgttgtcatttttgttatattgatcgatataggggatggtgcagaccgttcccctgcaacaacacattgcatgtggtgaacgccccgggaatcctggagactaggctgtgtcccttaattacgtaagcgttgataaaagagtcggatgcagcccagtttcaccatcgccaggtaatttctcgccgttttgtcgtaacgcgtggcgatgcggcgatattctttcagccgcccaaagcaccgttcaacgacattacgattgcagtacgcatcacgatcaagctgtgcacgtccatccgatgccattttctcatttgactttcgggggattactgcctttataccgttatttttcagctcgttgcgcaatgcgcgccctgaatacgctttatcagccagtaccgcatgtccacggcgtttcatgctgccgttctgacgctgaacgccaatcccgtccagaagtcgttgcgcgaactggctttcatgagcttgtccggggcttagcacgatatttaacgggaggccacttccgtctgttgccagatggattttggtgccaaaaccgccgcgagagcgacccggcccattatctccgtcgatatcgggatgtttttttgagcaccggcggcacatttcagcgcccggatattactgccatccagcgcggtggcagaccagtcaataaagccgtttgcatcaagtagcgaaagcaacctgttgaaaataatattaatcacgcctgactttgaccatcggttaaagcggttataaacagttttccatgacccatatcgttcgggtaaatcgcgccatggtgcaccggaacataacacccagaacatgccattgatgattttacggtgctcagcccatgggcgtccggcccgtggtgtggcgggttcaggaggtaacagaggcttgatgatggtccatgcctcatcgggaaggtcgtagcgagccatagttcacgtataggaaattgaaaaactaagcgatacccctgtccgcgccttatgccacgtgagcctttctgtttttccccaccactccggtgttttctctctctgtatggtcggtttttatacagtaacaggctgttttttatacttgcaccgattcccgggcacacttagcctgtgtcaggccggcatatacctcagcagactgatatcccgcgcgtttttcttcagcccctccacattcagccccgcaatggcccggcggtataccatccggccgccacacagcacgcattcgaacgggtcacgactcaggaactgtttcaccatttgtgcatagcacactttcgccactggttccggtttatccatccccagagcacggtacacctgcggcagcttctctccacacacccggttggcaaggaacccgaagtacctcaccatcttaaaaaacttctccgggatgtgctgtttcagcctcgcgaccagctcacgctgtgtcagcgtttccgtcgccgtttctcccgttttgtggtccaggtaacggaagctcaggctcgcccctccgttgtaatgtgccagccgggaggccgctatcggcggcttcttcagataacgacccaggtagcgcgccgtattcctcccgccggccgtttttttcgacatgtacacatgccagtattttcctccggctttcagcaccaggcttctccactgtgattccgtcgtgatatgtgacaacgactccggcatcgccagcccctctgaccacgctttcagaagccgttgccgcatattccacatccaccgcgaacgcatcgcgtctttcaggaagctcagctttttccactgaccatgcttattcagacatccacaggttacagacacatgtacatgcggatgccagttgagacgacggccatacgtgtggatggcgcagaagataccgggttcctgcccccgtttccgggcggcatacagcagattctccaccgccagacggcacacgtcattcagcagccaccggttgctttcgaacaccggccacagcgtgtccggcagggtgaagaccagatgtacccagtcgcagtcaggaagacgattcagctgtgtcgctatccacaggtctgtggccttttttccgcaggacgggcaggcacggctgccgcatgagttagtgacgtatctgacgtgggggcactcaggtttatcgcagttgtattccttcactcccagtatccgcgtgccgcaggccagcatttttgtgacagcctcaacctcgatatcgcgcagaccgcccgcatccaggaaggatgtccagcactggttggccgtgaacagacgtttcagcgggcggggagtaaaagcggacaacatgaggacggattaacccccggtatcctgtagccgggtaagggctgctgtgtcgccacggcgctccaggatggaggccgcgaccatcgcaggtggtgggtccatgggtttgagcatcttcatccagtcccattcacgttgtgagagcttctcagcaccaacagagagcagaacggtatctatacttttgcgggtcaacataagaggatgagtccttaataaaacaggtcctcagagcatacatattctgcggagcgtgcggcaacaacaggacgaacaggggcggttttcaatttgcagccgtcaggctgccgtggttcaatatgttgtgtaaacagacagttactataactcagatgattaagggacacagcctggaacccaggatgttgcagggaagagaacttcattcaagcttcatcatgagaagccgatcagccaaaatggtggtgtctatgatatggataacatcagcgtggtaacacctaaaaggcatattgatattcaccgaggtgaaccgccccgggtttcctggagagtgttttatctgtgaactcaggctgccagatcatcgtttccgatggaagcataataagctttttctgcttctgccggaggagtatggcccagcctttccagcaatcgtcgattgttataccagtccacccacgtgagtgtggccagttccacttctgcacggtttttccagctcttacggtgtattacctccgctttgtaaagaccattgatgctctccgccatcgcgttgtcatacgagtcgcctgtacttcctgttgatgccagtaatccggcttccttaagccgctgtgtgtaggccagcgatacatactgagaacctttatcactgtgatggaccgtgccggacggtcgacgggcccataacgcctgctccagtgcatccagcacgaatgtcgtttccatggacgatgagacccgccaccccacgatgtatccggcaaacacatcaatgatgaacgccacatagacgaagccctgccatgtgctgacgtaagtaaaatcagccacccacagctggtcaggtcgttctgccacgaactgacggtttacgcggtcgcctgcggcaacggctttccggctgatggtcgtacggacctttttaccccggagaacaccggcaagtcccataaccgccatgagacgtgccacagtgcatctggccactctgataccttcccgtaacaactgacgccagactttacgcacaccgtataccttgtgattttcatcgtatacgcgctgtatctctttcttcagccagtcatcgcgctgcgcacgggcactgcgtttatccggatgatgtcgctgttgctgacagtggtaatacgttgacggggcaatatgcagttcgctgcatagcggtccgaccccgtactgctcacgcagcttatccagcagtggcatcattttttccagaggcggtcaaactccgccttcgcaaaataagcggaagcctggcgaaggatatcgttactgcggcgcagttcacgattttcacgctccagctctttcagacgctgacgttcagcggtggtgagccctccatcaccgcccccggtatcccgctcatgctggcgaacccggacacgcagagtctccggcgtacagccaatctttggagcaatggaacaaattgtcgcccattgtgagtcatattcgccctgactttccagaaccatacggactgcccgttgacggacttcgggggaaaaacgagtatttttagtcatcctgtttacctctttctcaggaagtttagtctccaggattcccggggcggttcaggccagtacctcgtcagtcaactgacggtaaatctgctgttcagtctcacgcatcacctgtgcaccggcttccctctccgcatccgcatcaccgctcagacctgatgctttcagccggtcagccaccctctgagagtactcattctccagcatctcatacttctgctcttctgcctgcgcccagcggtcagcttccgtacgcttcagtacagcatgccatggtccccagagggagaaccagtccgtaaattcattctcttcacggcttctgaccatggcttcggcagtgcggaggtcatttgctgtcactcccgacacgccatagaaacgcatttccttcacggcagtggagagctgaagtttctctgcgagcatggtctggaaggccaggtagacttctatctcatccacaaaatggagagttctgactttatcccgggcaatgtcctccagaatttcgaggcggaacatttccctgcccagggagagcagagcgccggtatcattatcgaaaaggccttctgatgcctgatggaccaggagggttttccggagattgttccatgtgagcgcgacacggtcctcacagctctcagtggcatcagcagcaacagcgaaagactgctgtcgaagctccgcagaggcactgagtttttccagccatgcagcgacctgttcacggaatccggaggtattgcgtgcagagacggtatcggaaaggcggtcaaggaacgcggaaaaggtgttggcatgctcttcatgttcaaaagcatgccatatctgtgatacatcagattgtttgttttccgggaaccatgctgtcacggcatcagccagggggcgatggagtgtattctgttgtccgtcactcatggagaagtaaatctgcgggccgtggtagtccggcgaagaggttaatctttgcagggattgcagaacgcgagtcgacaatggattccctgaaatgttaacattcgtacaaatgggcaatgtgattatattttcgggtaatgaagttagtaggttattggaaacatccagtgccaatagttcttgcggtaatctggataatctgcgtagttgattttcacctaccttcaaatattttaagcgctgaggaaattctggtagttcctgtagtctgttgccggaaacaaagagttcttgtagtctatgaggcaattcaggcaagacagtaaggcgattaccttcaatacgtaatattgttagcgttgatggaagctctggtaacgtctccagactattaaaacgtgccgagagtgattgtaaatatggtggtaatgatggcagacatgacaaatttcgattgaatgaaatattaagcttaatcagttgagatggaagtgtcgggagtatattcaagttgttaccatttacatgaagttctgtcaaaagcggagaattttcaggtagggagattaactcattgttggaaatattaagttcacgtatatgcaaagggagtggtggtaaagaacgtagtttcagtaagcttaaattgagaacaggctcccgggtatccagacatattttcaataatcgaaccgcctggatgcgattttctgttgttgttccttccctagcccagtcatcccagatgcggtggtaatctctggtacactgttcttctaccgataaacgagatatacatgataagccacaaagggaacgaaaaaaagaatgattgttgatcggtttcatttattaacccctcactattcatatcttggtctattaatgcaaataaaacgagaaagtctttaaaaaaacaatcctagagctttatcggcgttttcattccagctggaggaatatgttcctgtctttccagtaaccgtcgattgttatatcagtctacccatgttaatgtgaccagttctactttcatccagtttggattgatctttatcccctatattttcaaacaatttttatcatttaactcattactgtctcgatattcccatagcaaacgatctggttggttagcccctcaaaaaagtcagttgcacgccgtaaatccctgccggtattgagcgaggatttacgggttgttgttaaggtccgtaatcgggtcacagtttttccggagtcaatccggtctgcggtttatgcttatgcgacgtgattatgaatggtgcagtcgtgagccgttttcagacaatcgcagggccagtacctcgtcagtcaactgacggtaaatctgctgttcagtctcacgcatcacctgtgcaccggcttccctctccgcatccgcatcaccgctcagacctgatgctttcagccggtcagccaccctctgagagtactcattctccagcatctcatacttctgctcttctgcctgcgcccagcggtcagcttccgtacgcttcagtacagcatgccatggtccccagagggagaaccagtccgtaaattcattctcttcacggcttctgaccatggcttcggcagtgcggaggtcatttgctgtcactcccgacacgccatagaaacgcatttccttcacggcagtggagagctgaagtttctctgcgagcatggtctggaaggccaggtagacttctatctcatccacaaaatggagagttctgactttatcccgggcaatgtcctccagaatttcgaggcggaacatttccctgcccagggagagcagagcgccggtatcattatcgaaaaggccttctgatgcctgatggaccaggagggttttccggagattgttccatgtgagcgcgacacggtcctcacagctctcagtggcatcagcagcaacagcgaaagactgctgtcgaagctccgcagaggcactgagtttttccagccatgcagcgacctgttcacggaatccggaggtattgcgtgcagagacggtatcggaaaggcggtcaaggaacgcggaaaaggtgttggcatgctcttcatgttcaaaagcatgccatatctgtgatacatcagattgtttgttttccgggaaccatgctgtcacggcatcagccagggggcgatggagtgtattctgttgtccgtcactcatggagaagtaaatctgcgggccgtggtagtccggcgaagaggttaatctttgcagggattgcagaacgcgagtcgacaatggattccctgaaatattaatcctgtaacttgaatctaattgtgaaaaactctccggaagagtggttatctgattaaaatgaaaataatattccttttgtctgatattgtttctatcagaaaattgaggaaaagtaaccagattattgcgctgagcacagagaatttccagtttatccggtaagtcgggtaatgttgtcagcctgttataataaacatgtaaaaatttcaatttttcgggaagagccggtaagttatatagcctgttattttcaacaaataatgatgttaaatggtttggcaaaacaggaaggttttccagttgattagaacgaacacttaaagttgttaatgactcaggaagttctggtatcacagaaagcctgttattatcagcattaagttctttgagaaatgcaggcagctttggcaatgatgttaatttattattttctacaatcaaagcagaaatatgctttggtaattcgggaagagacgtcaaatttaggtctgataaatttaatactgcttcttgattctgaaagcactgggagagtctgttaaaagcaatgcctcgctcttccccgggagaagagttcttttcccattcagtcaggattctcagataatattcattactggttgagtttgagttaatagagggggagcaagaaactgatgagtgtgtattatttacagagaacataatgctaggtcaaccttatatttgtgagaataaatatttaaaattgtatatttatacaaatttcacttaccatttccgatattaagttttaatagtttccattttacacataacagaagttagttgtaatttatttattgaggttttctatggaattctttttttattgtgactgcagtcaataataacactgaattcaacattccagtgagcaaggaaatgtaatccgagtaagtatatttgttttacatcatattttagccaagctcatttattcctattatagaatgataaattaaaattactgtaaaaaaacgatacacatttaatacgagggaggccggcagaaaaacggcttgcggtaaggcaggagaaatgtgtgccgcgaatgaagtccttgtatgactggataaagctccagaggaaaactttgtcaaaccatgctgaaatggtaaaggcgttcaactatatcctgaatcactggaatgcactgaatgagttttgccgggtagtacttccaatcattagaatatgtgcttttcaatgaaggaaccgatgactttttcgtggatttccacggaacgcgagaagcagattgttttacgtgccagccgcttgatgcgggggcggagggtcaggttgttacgttcatacgttgcgtgaagattttgccggtcaggtgtttctcctccggcacttctctggcgtagctgccccagtcatcgctggtcaccatgccaccatgccgatacagaacggagtcaacagggctaacagttcccgacaggtctcatcattccgtggcccgcaagtgtaagccaacacaccgccagttttagtgttgtacgcataccagggccagtgttgttgcgccttgctgccaacgaagcgccactgctcatcaagttcacagataagggcaacatcagcgtgagcgactggcgcagatgttatccgccggggcgagagctttttaatgtccgtatgacggtattgatgccgattttcagtgttctggcggtataacagccccctgcgccgttatgtgccatttcaacgatctgctccttaacgccgggttttctggcttcataagagtaagtgagctggaaaacacgtttacatgagcgacaacggaaacgctcatgccgggaacaactacggccatgtcgataaacttcatcggaatgacagcgcggacaatgaacagtaacggtagtcatggaaaacctcaaataccagtagattaccagaaacttctatcgattgaagacattaccttttgccgcaataggtggggtaacattggtgaaaacgcattacgccgggtggcagttggaaaaaaattatcttttttcggctctgatcgtggtggagaaaatgcggcaataatctaaatagctgcgcttaataccgctacacttttgccagcccatgtttgcctccggggaatgggctgacggtttccataaaatggcgaacttttttcaacagttgccacattgagctgcactgatgattacgcgttattgtgtcgtgaagtgcctgccatagccgttcaacatgattcacccatggcgagtaaaccggctgataaatgaccctgaacttcgggttctccttcagccagctctgtgtttcccggcttttgtggataatgtagttgtccacgatcagcgtgatggttttcgcccgacggtatgtcgctttaagccgcttcagcaggctgatgaacagcgccgaacttttgctgttgccgcccacacagctgactttacctgtcccgctgtgcagcgctccggccagataatatttttcattctgtcccggcgtgaccacccgtttttgctgtccgcgcagttgccagtccgcaccgattttgggattaagatggatatccacttcatcttcataaaagacctgatgttctgcgctgcattcgtccagtgctttatggattgctgccatcttttcatctttatgcgggtcacggatacgcagagttggcgcagcccttcgccacacaatccccgcagacggcaaccagcggcgaacggttccggcatttaactggcaaccggttatctcattgatttttattgccagcagttctgtactccagcgtgaacgctggtagccaaagtcgccgggagaatgttttaccagctcacgtaacagtgtgcagatatgctcaaacggccagcgacgggcacgcccggcaggtaatgatttcagtccctcaacacccgactgcgtgaaccagttaatccagcgtccaacagaggaacgggcgcagcagagcgttctggcaacgtcgctgacacggtcgccccggtgcagcatcagcatggcagtcagtctgcgggcataatttttatcgtgtgttttatggatggctttctgcatcaggcgtcgttcgtcacgggaaattggtgctatgatcggcattgctcagtccggttggtgatttgttttgatttggcgattgatcagatcgcacaatccgggctaagttccctcaaagggatctactattccgcgcagctatttaggctgtgtcccttaattacgtaagcgttgataaaagagtcggatgcagcccagtttcaccatcgccaggtaatttctcgccgttttgtcgtaacgcgtggcgatgcggcgatattctttcagccgcccaaagcaccgttcaacgacattacgattgcagtacgcatcacgatcaagctgtgcacgtccatccgatgccattttctcatttgactttcgggggattactgcctttataccgttatttttcagctcgttgcgcaatgcgcgccctgaatacgctttatcagccagtaccgcatgtccacggcgtttcatgctgccgttctgacgctgaacgccaatcccgtccagaagtcgttgcgcgaactggctttcatgagcttgtccggggcttagcacgatatttaacgggaggccacttccgtctgttgccagatggattttggtgccaaaaccgccgcgagagcgacccggcccattatctccgtcgatatcgggatgtttttttgagcaccggcggcacatttcagcgcccggatattactgccatccagcgcggtggcagaccagtcaataaagccgtttgcatcaagtagcgaaagcaacctgttgaaaataatattaatcacgcctgactttgaccatcggttaaagcggttataaacagttttccatgacccatatcgttcgggtaaatcgcgccatggtgcaccggaacataacacccagaacatgccattgatgattttacggtgctcagcccatgggcgtccggcccgtggtgtggcgggttcaggaggtaacaggggcttgatgattcgagcattttctgcccgcagtgccgataattcatcgctgtcagatttgtgaaccatgccctgctgtaaatctccgcatagattatgtctgtttgatctcagtcatccggtgtctggcgagaagacgcaccacggtggaagggtgaacccgaaacagacgggcggcatcagcacctgttttctgccccgatgtgacgagcgagacaatctctttttgttgctggggtgttaactttggtcgccatccaccaacccgtcctggcgtgccgctgcaagtccactctttgtccgttcacgaagcatggcgcgttcaaattcagcaaaggaaccaacgatttgcaccaaatactttttacgaggttcttttcgccagtcctgtaattccatactcaggtgtccatctatgtcagatgggcgtttaagttaccaattatcgcttacttacggggtgagtatcgtaaaaaagcggctctgcaagtcctcctaattggtgacattaccgaccatttttcttccttatgttttgagggtatgtctttaaatatttaaagaaaactttagaaaaatacgatggtgaactgtaacctaactcagatgatatgacagaaatgggagtatttgtccttcgtaatttcctgatagcttctgtaagctttattttatgtacaaatgtacttatagaaccgacctctttgtacattcttctttttaaagaagattcactcattaaaaccgcactggccaactcggaattactccagttacgggagtaatttgccatcacaattctggcgattttttcagacacagaaacaaatgatggtgcatgaagaataagcccaacatttttgttttttttcagaatacatgaaagcaaataagaactccgaactttatcgtaaatggcatctgtctccgtctttttattatcagtgcactgatgagcacactgagatattaaccaggacattgcatagggttcatctccccccccactagaaaaatcaatcaaaacataatcgggagatataagctgagctttggaaattttttttaatcgttcaatgtcatgatcaaataaaacgttatgttcacttatttcctctgacgtaaatcgcacaacacaaatatctcttctttgaagaacagatatatccctgtcaaataaaagagaaaaaggaactaatttattaataaatataccattgtattttgatacataaataacagtattttggacagatatgtggaatggttcgcctgatgtatagataaagtagaaaaacggcagagggatttcaatgtatttaaatattttcgcgtgcgtccctgagtaagggactacaggaggtttattattaataaatatctgttttttcatatcaaagcttgatttttacttatacatctttagaatcaatcagattttgtaaatctgcgccatcatcacacctgatggtatgagaaaagcagctcatcttatcccgttatcaaagcctaagacctatggtgtccagtcacctgacaagcacatcctccatagttttcggtatcattttttctacccaaagtatcaactttcagaaataccgcccatatttaacaacatcaacgaaccgtccaagccgagctgaaatcctcttcctgaaacaactctgtcagcccgctaatctgtttcagcctcagcacttcatcctggtccattcccagctctgtaccaatacgttcattcgtccagcccagacgtgaaagatccctgacaatatcagacattaaagtgatctggtgtttccccctggcccggttatgtcgaatggtcgcggcaatacgtgcggcctgtccctttctctccggattaatacaggctacaggcaaccagcccctcaggcgttttcctgtatctgcttcccgccccagtaactgacgatgaaaaccatccaccaccagataatgattcttctcctctgacaccaccaccggttgtgtgaaaccatctttctccagagactgtttcagtagttttttctcacctagtgccatgacattagggttgtaatcgttggccaccacctcatccgctttcacccacaacacacagtccacaggctcctgtgcaaaaggactgacctcatgaagcactctgcgcagatgattcagagcctgtatgctctctttttcagttcgtacctgactgaaatactgacgaatcatgttgccaagtatctccggtgtcacagaatcccccattccttcctcctttccttcatacgctgcaggtaacgttcatagtgccggggtttgttcggactgaaggagagagtccggcaccagaaatcattctttatgagtgtcttacagatacgtcgccaggacgggatatcccggctccccaggtcattctcctgttcatccgggatgtcatccgggaagccccgcgtctgataccagcgcaggtagacagcaattttattacggtaatgttctgccgttctttccggcatcacatccagcaggaacatcgcatagctacgccaggtatgatgaggaggctttgtgatacgcttacgcagggcaaaataggcaccgctctcattggcataaagcgccccgctggcagcacccgacacccgctcacacatcctggcccaagtttccggctccagaacatgataaagccacagccccttacgctgttcaggaccaaaaggctcacagacacgcatgttgcgtaacggcacacaggcacggtgcatcaggtcatacaggggattgtagatagctcgggttctggcgttatatatccagatatcacgggctttccagtcatacaacggatacatggtgtaataaaacccttcaggtgatgccgtagtccagggtttatcatcggcatatctctgtttgcgacgggataccagtcccataaagcggttgagggactcatctgcccggactccggtcagaacagccaccaggtatttatccctggcaaaccaggaggaaaacgccgggataaattcctcaaaggtcatggcatatcggtaaaagggaaaatacgccatatccgtaatagcatattccggtggatggcgaacccaggtcacaccaggctcccagcatatccactccggctgaaactgagagacaccgtttaccgtagtcaggggaagggccacccagtaaaacctgtccgtcacatccaggtacatcttctgcatcttctgaatatgcgtaatggtgcattgatattgtgcttcccagtcaatgaacaataccgaaaaacgacgcctctttttgcgggccacttcggacacaagatggaaaagcacggtagaatccttaccaccagaaaaggacaagcagacagaaggaaacgttccaaatagccattcaatgcgctgaatggctgcagacagaacattttcacctgttttgattttcggtatggttcctgacataattgaacaaaatattcagtactggatgctattcagcaatccttgattagtaaatcacaaaaaacatattaataatattctggcacaaactcatttttcaagggggaatgcccataaaaaagcagaataattaataatattattctattttttgtgagctataaagcccatggagagcaggaaaaaactcactcaaatagtgatagttttaatttttatctatgtataccacttattccagccactcccgctatcaacacaactccccaatattacagtaaccgacgttccttatctccggattataaaacgcactcctgctccttactgatattcaatgtttactgacagtgacaccctccagtgttccggcatctcgccgttcagccgtaattcaccactgccagccacagtctgtgcaccatacacgccttcaacctggcgcaggaagtcatcactgaaatgactcacccctgaataaccactgagtgtcattgtctgaagccagccggggccttccccactgaacatcttctgcagaacgcccggaatatcagaaaccttcatggcagtgctttcacctggcaaaccattcgtattcaggtcgtcatacataccaacatacaccggttgtccgccctgcacatggcgcaacacccccacagcctgaattctgaaaacaaatgtgcctgcccgggcattattatctccggtgacaggtagcgtcacttcagccatgccaggcgctgtccatacaagtgaaaacccctcagtgccttttccgtaactgacccggggtgaaagcccctcacgtcctgccggtgtggtcagggtggtttttcccagtaaaacggtcatcgccggcagtgcgacctgaatcccccccccaccgccactctgtttaacctccagacctccatttccctgcccgactttccatacccacgggttacgggtactggtcaccgggccttccagcttaagctctccgctgaaattttctcctggcgtgttccaggccagtacacaggaaggcaggtggctggcagcaaacagaaacaacgtcacctttttcattctcttctctccttatatccctgcaaccatcactgtactgtaaccgtcacattcagccccgcctgccatgctgcaggtgtaccctctgccggcaaccgcagttcaaaatccgacacctcagaggcatatgccgcagccagatccatcacccgaccgtcagtcagtatcccctggttgataacctgtccgttatccaccgcattcatgccattgccaaaattctgattcatcagtaacagggaagagagtcgggctttcagcccctgtggcagacttcccggctgtacatccgtcacactctgtccactgaccagcgacatgccagccggtacggatgccccgtcgtcctgtctgccggtactgaccgccattccctgattcagggtaaaaaaaagctgtcccgacacgttccccgtctccgggtcacgcaccgggacagaggcgcgaaagtgctgtgctgaggcaccatttccttccgtcacagtaaaaggctgtccgttactgctgaaagtaataaatggcgtgaatccgggaccaccgcgctcagccacatcatgcagatgtccctccagaaatggcagagcccctttatcatgcaaattaaaaaccagttgcccgttctctgtacgggcatcggcagtatccacagaccaggtctggtcgggagaacttatctgccaggcccatttgggtgcttcatcagtaacatagccattaaactgaatttcgccgccatcaagaatatttgcatatggcactgccgaataaaatattgttaccaaaataacagcagaaaggaagaatttataatgatgcgttattttcatcggcattcattcctgtttatccggataaaacaaaacagctcaataaaataattttgttaaaaaaagcacagaaccaatatgcgtcacaagacactccaacagaaaaaacacctgttctgacataccggttctgtatattcaggtcgggtcaccggaaactgtgttctgaagttttaatcagcagtaaacgggtaagtgcttactgctgaacactgctgaattacatgtaggagataacaattggcagagaagctttccacggaactggggtagcagacgttatagtctgatcaagaactagttccatggggatatccttggcaaggccggatgcatagtaacctcggtaaagcttgtctccattactgaagtcggtttcatctgtatcgcccgccgtgtaaggaattttagtatcccacgagtcagcaatactgctaaaaaggtgggtagcccaagtataggcagcggcgtcactccagacacctttgtcatctttggcaactccaccaaagaaagctttaccttttgaactggcgaatagcatgttactctctaacttgtcgtcgccttcacccctcttttcgctggcctgacaggcaactcgcattacgaacgccagagtaccaattttcttaccatcagatgatgtgacattaacttgcgtaggtaaattccccttatccatataatccacatcagcggctttattataatcgatttgtggggcaatacccttttcccccatgaaaccagtcgggttgctacgaatgccaagtatcggtatgtctttttctaaaagaatgaaaacattcttctggtcaggtttaacggtcacatcaagcgctgttaccggagcacctatttccactgcccacggtactgttttcgcagctttgctcagagtaccacccatgctaaaggtgccaccgttgccaccaaggccagagggattccacgccatagcagagccggatactgcggcagaagcagccacagtcagtgcaatcagagtctttttcataaaataatcaccttgacgtttcataaataataaactgaatcggtaatccttattttcccggaattaccgcaaaaccatcccctgcggattcatttaaataaccgcatttcattaaacacagtgtttaactcagaatagttttacgttattcccgttttaaaagacggtaaatccctttaccaatatgacccgctccggtatcacgggatgtactgcatttataaaggaaatgaatcctcagtcctccacctgcagcgctatctgcagatttccccggtaactgacaggtacttccagctctgccggtgcatccgttcttaattcaatctgcttctgtgcttccggcaaagttatcaccatatcctcaccctgctgtctggcgctgattgccactttctttccgtcaaccatcagggccgtcttcagggtgatgcgggcttccccgttatccggcagcacctgtttcaccgtgatatacgccaagccaccagatgctgctctggattctgcattacggatggtcaggcggtcgggaagaggcgtcgtgtcaccactccggtacagcaccggatctgtcggtatgcgggttaccttcaccacgctgttacccaccacctggccattcaccacccccagatactgcgctttctcgctgtattcagcggcaagtgttgactgaatactgagtgcactcagggccaaggcgactaccatcattgttttcttcatttattacccctctgttccgtatgccggagggcgattattttttatcagatgaattaccctctgtatcagatgacttctgtgcagacgatattgctgatttcttctggttaatcgtccatgtctgcagttcaccgtaatcattcagagagtccactttcaccacatttcccttcacctgtacttcatcacccggcatgaacatcagcagtttctgcgccgtctcgttatccgcggcaattttctttccgtttccgtccagcaggctgccaatcgcaaaaatgtatggcgtggtgttcaccagcatggtcctgccatccgggcggctctgcagactgatgccttcttctgcccccttgcgaccttcaagcagtgccttcgggcgatagaacagtttcagtttcgtgcgcagcgccactgcaataccgctgccctccagagccggcggaatatcctgcaggttcagccagtacaccgactctttatccttcggcagatggtccgaggccatgatgatacgcagtgtctgctgaccattcggcttcaccttgaagaaggacggcgtcaccacaaaagtcggacgggtgtctttctccacgatattgtccacccatgcctgaccaccaaaggtacgttccttgtcattgttggtgaccgtgatggtcagcgcatccttgtccccgcgaaagatatagcgggtctggtctgccgccagggaagccaccgcactctgacagctcatcatcagcgccagcgtcattcccagtgcttttgtcacctggttagtgaaaaacgtcgttactgcgttacgcttactcacacgtaatctcctgtagtttctcagtatcctgcagtctggccgcaggtatccggcactggcctagggtgatatcacccggcgcatccaccgtgttaatcatcagcacaccattgttagctacaaagcccagcggtgtgttcttactgtcacgtgcccaggttccccctggcacaaactcaccgtcacgctgcttcacctgcagcaggtaacgcttaactttcagggcatcaaacggcatccagaccaccgccttgtccgtcggcaccaccttctgactggtgttcgtcagttcggtgctcagcggcagtgtgcccgcatcaatcgtcaccgtgttccagtcatagctgttcagcggtaccaccaggttgccgtcactgtcagtctgtccgtcaccggacgtcaccttcacacccggtgtatccttcacgttcaccacggccacggtgtccccggtcgtgcggctgaacataatgtctttcgccatcgggacgaccagtaccgaaccgctgactgatacagaaccactggttccgccggactgtgagtggttcagggcaccattcagataagcccggtcaccactgtacgacgcgtttagataactactcacaccaccaccgctgtcacgaccgccgcccaggctatagctgaaacggtcattcagcgagccggacacgccagtgctgaagcccgtgccaccgcctttactggtactcaccgatgtactgctgctgtagcgacgctcaaacagcgtgaatggcaccgagatagaggctgacgccgacctgttattctctccctgcgaattccgtgagccggaccagcccagattcagactgacgcccttaatctgtgtgctcagggtaccggtaatccctgtgtccagaccattgtcatcgctgtaccgataacggccagtcatctgtgccgggtaataactccgctgccagccggacaccgacagactgaacagcccccccaccgactgactgataccggcattccattcatccttcggccggcgaccatgatttgactgcgaccacagctctgtcgggtcaaaggaggacatgtcctcatattcctcactctgccgtgaccagctcacccgcagtccggtgttcgtcatctccagttgtttactccaggacaactgcaccttatttccgctgcggctgccgtcacggtattccgccatggcataagccccgtcagcagacactgcccccagataactcagccccagaacagccccggcgctaataccctgccagtcccggttaaacacgcccccggtgcgcagcgtcagtccatccagaccgtaaccgtatgacgccgcaaacacgccgcctttcaggtcactgtcatcgtcagacaaaccgactgccacgctgaactcgtgttgtcccgggtttaactgcccggccatcaccgacaacgggaagagctgagtctgctcacgaccgtcctcgccggtgattttcatggtcacatccccactggtgtacagcggcacatccgtgatggagaacggacctgccggcaccatctccgaatgcagcaaccgcccgttctgtgtcagtgtcaccctcgacgccccgttcgcaatgccgctgaacaccggggtataccccagattgcccggcttcatgctgttgttccggctcagcgacactccgtacgttcccgtactgcccagcagactgctcccggtggatgttttcccgaccgtcaggtccgcactcagtgcgcggatggcccgggtggccgtgaacgcgtttatcgtggcggagttatccccgctgtcaccaccgctggctgaggcagaagaattcaccacccagtgcccgatattggccttcaggtctgccgagccaaaggtcgaggtgttattccggccgatgttggcgttcgcgttatagttcacgcgaaatgcaccagtcccgtaatcccagtccacgttctccggcatcttcaccagccccttttggggaatggacagcgccagactctgggtggaaacatcaaaatccaccttcactgacggcgcttttgtcagcacataacactgccgtgtggcgtcatatccctcacgaaagtaatctgcactgacgtaaatcccggccttcgccagccatgcctctgtcaggcacagttcatcactgtcctgtggtgtcacatccagtatctgtttcccggcctccttcccgttcagggacaggtcaaccagataacgtcccggcgcatagctgccgttcagcgctgcccagacttccggattaaccccgccccccccccggataaaggacatatccagtttttcacctgcaacacctgtggtactggcgaaagcagctgatacagcaagcgcggcccgacgggaaaacaaagacgaaaccagctctgcaaccaccttctttcctgcacacacaaccctttcctctgttactgatacgtgaccacaaaggtcagcgcccctgtgtattcccctgccttcacgttcgccgggaacaccggctgtgcacgaaaacggaactgtcccttaacagcaaactccaccgggtaattcaccgaatgactgtcctgagtcagacgtccgcgataatccgcctgtgtatccaccgcccgtacatccacgcgaataccatcaccagcaccgcgagtgaccacgccctttgcgtccagttggcccgggttaccaaaatccagagttgcgacctgccccgctcgaaaggctgaacaaccttgaaccgtagcgccggactcatacaagcgcacaacaaagtcacgtggcaatacaggcacgccactggatttacggtaagtcccgaatatcagtcgaccactgttgccgaaaccatccgcatccaccaccacatgacaggcagaacctacaatatccacattgaaaacaactcgctgcccccccccttttcccgtattggaaattaataaagcagagacaaacacaatatatttaatactcactaatttagagttaataactcacaccccatttttatgaaaattacataaaaataatttagaaaaaaaataaacaaatatccacccgttaaaaacaaaaaaaccaaatagtgccaaaacatgaaccatttttactatttatacttgcgttgatttggcgtttacctgcgatgcaattatataacaattgattaatactttgattaattaacatttacatacattaataatgcaactctattgaggtataatatataatacgggagttgacatgggatcccgtatatcagcagttatttttactcggcagttcattgattattatcaataataaacgtaaaccacgtggccgaaatattgatgggataacaccaagaggaaaagggtgatgactctaggaacatatgtttttcgataaaagcaccgataactttttcatgaaattttacagagcgagagaaacagatggttctgcgagccaagcgtttgattcgggttctcaggggcagattgttgcgctcaatgcgctgagtaaaaatcttaccagtcagatacttctccttcggtaacttgaggtagcctgagtttaacggacactccttcctgaaatagaatggcatcagaaggagctaataatgagcagaaaaacccaacgttactctaaagagttcaaagccgaagctgtcagaacggttcctgaaaatcaactttcgatcagtgaaggcgcttcccgattatctcttcctgaaggcactttaggacaatgggttaccgccgccagaaaagggctcggtactcctggttcccgcacggtggctgaactggaatctgaaattctgcaactgcgtaaggcgttaaatgaagctcgccttgagcgagatatattaaaaaaagcaacagcgtattttgcacaggagtcgctgaaaaatacgcgttaatcgaacaatggcgacaacaatttcccattgaagcgatgtgtcaggtatttggtgtatccaggagcggttattacaactgggtacagcatgaaccctcagacagaaaacaaagtgatgagcggctaaaactggagattaaggtggcacatatccgcactcgcgaaacatatggaacccggcggctccagacggagctggcagagaatggcatcatcgttggtcgtgaccgactggcacgtcttcgtaaggagctaaggctacgctgtaagcagaaacgcaagttcagagcgactacgaactcgaaccacaatctgccagttgcgccaaatctgctgaaccagacgttcgctcctacagcaccaaatcaggtctgggtggcggacctgacgtatgttgccacacaggagggatggttgtacctcgctggcatcaaagatgtttatacgtgcgaaattgtcggctacgccatgggagagcgcatgacaaaagagctgacaggtaaagccctgtttatggcgctcaggagccagcgcccacctgccgggctaatccaccactctgatcgaggttcacagtactgcgcatacgattaccgggtcatacaggagcagtctggtctgaaaacatcaatgtcgcgtaaaggtaactgttacgacaacgctccgatggaaagcttctggggaacgctgaaaaatgagagcctgagccactatcgttttaataaccgggatgaagccatctcagtaatacgggaatacattgagattttctacaatcgtcagcgtcgtcactctcgtctggggaatatctccccggcagccttcagggaaaaatatcatcagatggctgcttaaaaaaagaacaaatggtagtgtccgctattgccagtacacctcaggcgtgtcagatcctgctctgatgtactgactctctcatagatgcgagcaattttcatttactgtagccaaaatgagtataggtaatttggctatcgtaaaaaaggtggttaaaaattacaacctgcaatgtaaattggctagttaatttttggctatttaaattgacttcttaacggcgtttactgttccattgctccccaacccccaagtacaggcgactcgtatgacaacgcgatggcggagagcatcaatggtctttacaaagcggaggtaatacaccgtaagagctggaaaaaccgtgcagaagtggaactagccacactcacgtgggtggactggtataacaatcgacgattgctggaaaggctgggccatactcctccggcagaagcagaaaaagcttattatgcttccatcggaaacgatgatctggcagcctgagttcacagataaaacactctccaggaaacccggggcggttcactgcgtgtctgggttcgccagcatgagcgggataccgggggcggttcacacgaacataccacagacagttgattttggtacgcccaggcgacgtccggccacaacccgtggtaaatgttcttcgaaatgaaggcgtaaagcttcagtgatccaggtccggtattccatacgatagtgtccacttcaaaatgatggacattatttttgtggaaccggaggtaacagaccagacggtttaaatgagccggttacctcaaattcagtgtatcaccacgagataatattcagctttttatattcttcataatttctagaaaatcacttccattttttttgtccaagtgttctgctatttcttcaggtgtaatattttcattttttattatttccatataagtttttattgcctcaaaattcctattttgaaaagctataattaatccagataccccatttgggccttctgctgccagtaattttgatgcctgatgtggagtaaggtctgcagtgtgaagtattttcccataagcaataattgtttctacacatccattatgcaaggctacaaacaatccagataaaccattagaatctttcgcttctaacagctctgcttttttatccaagttaagttttgcctcattcaagatatcaccataagctaagattgcatcagcctgtccattattcaatgctaacagaagtccgggaacattgtcatatctccttgaggccagcaaatttacaatatcctctgagttgaggagaggggggctaagaatgagctcaccgtatgcgcgtatagcatctgcatgtccattttgaagagcaaacattagtccgggtatctgtctgtgtacttcatcataataataagcggtcagcaaatttttcagcttattgtattcgatatttattgctctcttcttgagaagccttccgtatgttttgatagtttctgcatgcccattttgaagtgcttgggataaacccgatattcgtcctcccattccttccgcagttagtagttctaccgttttatcaaggttaagtccagaagtttccaggagttctccgtatgctaagattgtatctgaatgcccattttgtaaagctaaaagcaatcctgacaaaccatcgtcatttttggctgccaaaagtaattctaacgtattgtctgaaaggtcagatagttgtacgactcttgttactttttggattagctcagtcaatcccatacgcatcgcgtgataaataactttggggtgaagtaaattattaggccatctgattatggaggacatgcttgttggagtatgtctgtccacaaatatagacataccgtcggatataagaccataacatttctgatgtttttcatcaagaaaattatctacagtcagtttttttattttagctaagttaaatttgttaaactctgctcgtaaatgagttgctgtatggttgggatcataaagctgaatgacaaaatcccccccatttttagtttctttaatcttcaagcctaatgccatattgtgatttgatgtacttatttctagtgctctgcttttttcggaatgaaaaatcatttcattgaaaatataaaaaataatatctccccatgaaccgttttcaacaaaataaataatagcaggcacattaatagccttttcttcttcactcactgaaacataatttttaatttcatccggaaaagcaaagtgatgataatccacgtgaccactacttttgttaaactgtgctatccagtaggaagctaaatgtcgacagacaataagtcttccagagtgatcggtaacttggcaatttagatttaataaaaaactcctcttgtcagaaaaatatggcaattgttttgagtgcaatagagactggattgtctcaacagagacgttgtctttatttatcatattttgcaaacatattttccttgatggaattaaatttactgatggcatatgtatatattcctttcattgatgggttgtgacgcagacttagtgttgttgtactctttgccctttggatgattgatggcaaagattgatgtggtttgtccccgttactctgaaactcaggggggtatccggaacggccattccagttcaggggggctgctctatcgctgtaagcagtgcctgaagaccttccagctcgactaccgctacaacggggcgaaacccatcaaactttccaactattatcacattgagccatactgacaataaacaatatatcgatattttgtgcaaatactgccttttgctaaacgatattcaattttgattaaatatatttattgtcagatgttgcaccatatttcaataatagttttatcatttctgcattctcatattttattgcgttatccaacatacaatccccactattagtttttttgaactttgtgtttacatcaaccaatcccttattgataaaatactcaagtactttacagcttgaattatgagcgctaagtagatactcaatatcatacaactcatgctttatggaaataacatcttgtctagttatctcaaaattagataatatatataaggcgacatcctctttttgattagcaatcgaaagaaaaagagcctggagagcaatttttttgttagtgacctgcttgttaatttcttcgtaattagatgctgtaacagcctcctctaaggatatttctctaatcttaacttttttgaagttttctgtacttaccatcctaggtatatggtactctggctgaaaaacaaagtttatgattctatccagatccacaggggcaagattttttccatagcaaaactccttgaagctttggtcttcactttttctaataaagtcaatcaggtataatccgagccctaacttcatatctttagtattgaatattggtacatctataattcctttacttccatgtacgtatcgactaacttctttattaacctctgaaaatttatctaaaaatgattggtgctcatggtaaaaaacaggtggaatagcattatcaaagtgatcggttaatgtcatatatccatatggagagtcatgatcaatgatatacgcatttgcaccaaaatcaacagtgtgatgttttgtattcagggggtgttttttctgatggctagagatttcaacgccaaaaaacacaaagtcatgattagctattttttttatatcatcagcgtatgtgtttcgttttgtgcatattccttcagactgaattttattgaatgacttaatgtttaaaattttatcagacaaatcagaattactttgatgtctgaagattaaatcagaattaataattagcgaaagcaaattgagttcctctgcagaaagaggatgaagtttttcccttatttcagatatataatgcttcgataatctgtcaaggtgctcaatagccttaaatgaggctttagcgtctgatggtgcatcaattgctatatttaaagatttaaagttagagccttgagagaacattctactataactctgtgctgctatagttttcctaaagaaatctttaataatatttgcactatcttttaaatttttttcatctggcatagtttgttgtgcagcgtttcgcactgatgtcacttttggaaacaatgtatgtaatctgttatccatagaatctatattgcattgggtatttgctgagttcagtgtttctgatatattcataaaatgaaccttatatgaaaacagttttaattaatatctttccctacaagatatagcagggtatgaattacaatttttgagtatatatacaatatactgcatgtaattagataagggtgttatatacttttgatgttatgtgatgccagatatttcaaaatatctaaaatccgatgcttaagaactcaagttaatcttttttgtgaaattattttcataatttaataactatatcaattttattgaacagcaatgaattttactataaaaaaacgatactttctctgattcctcttatggagtgcttccagttagtggaatgtgtattctgcaatgaaatcctcgatgattttttcatgaagctcaactaagtgtgaaaaactgatgtttttttaatagaggttagtttggccatatataattattgtacttttgcagtttttttgttacttcccttatgagttcaaaatcgccttaatggagcagtcactgcaacccagagcctgtgtggcgtagatcgcccgtgaaaatggaatctgccgttcggtttttgcgcgaagtttttctgagcttttaccgaactacatacgctgcagtttcgcaaccagcgccttcagtcggttgatttcggaagcataagccgccatccgctgtgagagcaggcggttgtattcagccatctggtggatagtgtcctgttgcgtctgcaacagtgcccgcaggcgggcgttcacgtataggaaattgaaaaactaagcgatacccctgtccgcgccttatgccacgtgagcctttctgtttttccccaccactccggtgttttctctctctgcatggtcgattcttataccgcaacaggctgttttttatacttgcaccgattcccgggcacacttagcctgtgtcaggccggcatatacctcagcagactgatatcccgcgcgtttttcttcagcccctccacattcagccccgcaatggcccggcggtataccatccggccgccacacagcacgcattcgaacgggtcacgactcaggaactgtttcaccatttgtgcatagcacactttcgccactggttccggtttatccatccccagagcacggtacacctgcggcagcttctctccacacacccggttggcaaggaacccgaagtacctcaccatcttaaaaaacttctccgggatgtgctgtttcagcctcgcgaccagctcacgctgtgtcagcgtttccgtcgccgtttctcccgttttgtggtccaggtaacggaagctcaggctcgcccctccgttgtaatgtgccagccgggaggccgctatcggcggcttcttcagataacgacccaggtagcgcgccgtattcctcccgccggccgtttttttcgacatgtacacatgccagtattttcctccggctttcagcaccaggcttctccactgtgattccgtcgtgatatgtgacaacgactccggcatcgccagcccctctgaccacgctttcagaagccgttgccgcatattccacatccaccgcgaacgcatcgcgtctttcaggaagctcagctttttccactgaccatgcttattcagacatccacaggttacagacacatgtacatgcggatgccagttgagacgacggccatacgtgtggatggcgcagaagataccgggttcctgcccccgtttccgggcggcatacagcagattctccaccgccagacggcacacgtcattcagcagccaccggttgctttcgaacaccggccacagcgtgtccggcagggtgaagaccagatgtacccagtcgcagtcaggaagacgattcagctgtgtcgctatccacaggtctgtggccttttttccgcaggacgggcaggcacggctgccgcatgagttagtgacgtatctgacgtgggggcactcaggtttatcgcagttgtattccttcactcccagtatccgcgtgccgcaggccagcatttttgtgacagcctcaacctcgatatcgcgcagaccgcccgcatccaggaaggacgtccagcactggttggctgtgaacaggcgtttcagcggtcggggctgaaaatcaccaccacttcgggtcatccgccttcatctccgcttctgtttcgtataaatcaaaacgacggcaggtatggcagaacgtgacgtattcgtacggtgaatccgggtacattttgtggaaggtttcccagaaacagaccgtgcaggagggatgaccggtgataaagtctgcaaatacggactgataagggtgattattggctctggcgacggctttcagaacctctttcaccattctggtgtggactttctggtgctccaggttgtgtgacatgggaactcattctggatggttactctgaaagctcatattctgccacaccccgatttgcagccgccaggctgccgtggttcaggtaaacgcctgttcgataaagcattacccaacgacgaaaacaaactcaggtcgctaatatctgacctgaaacaacatggtcagatactgctggttgttgatcagccagctaccatcggtgcgttacctgtcgccgttgcccgctcagaaggagtccttgtcggatacctccctggactggccatgcgccgcatagccgacttacacgccggtgaagctaaaactgatgctcgtgacgctgccatcattgccgaagctgcccgtaccctgcctcacgcgctacgcacgctgaaactggctgacgagcaaatcgccgaactctccatgctctgcggcttcgatgatgatcttgccgcacagacaacgcaggccagcaaccgtatccgcggccttctgacccagatacatccggcactggagcgcgttctcggtccgagacttgagcacccggcggtactcgatcttctccagcgatatccctcaccagaaaaactcgcttcgctgggcttcgctgggtgagaagaagctggcagcccagctctgcaaacttgcgcctcgtctgggtaaacgccttgcagcagacatagctcaggcactggccgaacaaaccgtcgtcgttcccggcacgaatgccgctgccgtagtactgccacgtctggcactccagctcatcacgctgcgtaagcaaagagacgaggtggcgcttgaggtagaacagcgagttcttgctcaccctctttacccggtcctgaccagtatgcccggagtcggtgtcaggaccgcagccagactcctcaccgaggtcgcctgccgcgccttcgcctctgccgcacatctcgctgcttatgctggccttgcgccggtaactcggcgatccggctcgtcaatacgcggtgagcatccctcgcgacggggtaataaagctctcaaacgggcgttgttcctgtcggccttcgccgcgctcagggatccgctctccagggcttactacacccgcaaaatgagtcagggaaaacgacacaatcaggcgcttatcgccctggcgagacgacgctgcgacgttctgttcgccatgatgcgcgacgggactttttataccccgcaggggtcataacatgcttgacaacttaataggggcacccccctaagccgtctatgggaaggagtctgctgttattccagagaatggtaattcagtcatatatttctccatctatgacttgtcaataccatcctgatatttgtgggtcagcaatatattctactaaaaggtatttaaggtcattgcgtccctgcactcgaataatggtgaagaaagcatgatgaatatatcacgataacgttcagtatgtatcgaggacagatggagttactcctctctgaaatagaattactgctctactttttctgtttccagaaattattagtaacaaaccctgatatattcttttgaaaatggagcttaaacgagtctgaaattaacttcagatattagaatgccgttggctttgtttttcaacaattcctacttcatttatctttgtcaaccattgcatttctgtagcagtgaatttcaacgttttcgtccggttggcagcaggtatttgtaattcgtatatagctgcttttttattgaaaacagtatatccatctgtagtaacagtagaagataataatacaggatttgatacttcaaggatgatattttcttgtttttgtggaaggcaagggaatatagtttgtgtaagcatattgcttccttatgttatattttagtaaagaatttattttttacattatagtcggctaaatgaagtttactgtaaaaaaacgatacttttttcgtaagcgatgagatgtggagccagaaaaatggcagcgttacgtcattgaactgtccttgacccacgatattcagacagtcgttgtatcttaagttgacattacccgctgacgtcgatattcccttggtgagagataacccaacgcactatgcggatggttttcattgtaatgctcgatcgccactgcaaggttatgcaatgccgttcttggtttcggcatgaacgcgatgcagtcttccttcatcgttttcacgaaccgttctgctattccattactctgagggctactgactgccgtcgtgcagggctccagattcaactctctggcgaactgccgtgtttcatgagccgtataagccgaaccgttatcggccactggattggccgttccggtcctttatcgccatatcgctgacatgacatcctgcaccgttgctttgtcataatctcccgtgcctgctgcccagtctatggcttcacgatcgcaatatggaacgtaacgctgtcatcgggagaatcgtcaatgtggcattcggttaacgctgatatgactttccgtgtcaaaccggaatctctaattttgacgattttctttttaggtttaattccctgagggcgacgtagcagtaaatccgacggtgaatcacgctgatattcgcgggttggataccgcattacagaggtccgtcatatgagcctgccactttcttacgtcgcgaatcagctttgttccgccgtcgccaagcgctaacgtgcgttactcttgtaccgggctacgtcgccttcagggaactggttattctcattttttatttgtgacattgtggttgttgtgcgacagacattgctattcggcccatatcccagataaaacccgccagctcacgtgcaacagcaacaattgtaatattgacattctttcctttggcctgaagttttcgatacctcttacaaagcctgagttgagcatcccatgctctgtcaatgacggggcggggtaaattttcctgccttttttgtattgccggactgatgcgtgccggatgacggtaactccacgctgcctcgaccaatagctttctggcatagctgtttccggctttggtaatgctgccctgatgtctgcttccacccgatgaatattctgatggcgtgataccaaaccatgacatcagttgtcgtggatgttcaaaacgggtcagatcacccagttcagaaattaatccaaccgcagtaataaactgaatccctcgcatggcttgcagcgcctctactaccggatacagcctccattcggtaacggcctccttcagggcggattccagccgttcacattgtgcctgccgatcttctattgtacgacggtgttcgtcaaaagctaattgtcgccagggactttcgaatgagtatttgctgagccagcgtcggtgtgctgggccccaatctgctcgcccgacataatggaccccatgaaccagaagaaacgatttcagacgttgccttgcatgccgtaaatcatcgcgggcagatgcccatgcccgggccaaatcccggaatgcctcatcttcaatgccgggtacgtagacagcagaaagatctcctgcacgcagtgaacgcacaagtctgatggcatcacggcgatcggttttaacccgctcccccggttttttcgggataagtgagggggcgcagaccatgcagtcaaaaccggatttcaccagccgacgataaagtccataaccacagggccccgcttcataaacgatactgacctgcgatgactttgacctgagacgtttacaaagattctgaatatcagtaggtgatgtgccaattttacccatcagttcaacaggttctgaattgatagcataagcgacagtaatcgattctttgtgaacatctaacccgacgtacaatgtgttattgttgttcatgctggcctccgtggtggaaataccggggtgtagcaacagctcacccatgcggctctgacaacgatgttaacccgcgttaaatatcccacgttggccagcatctgtctcacggaaagtcatactgccttaccaatatcaacaccaaccggatttgtggagcctccagtctgaaggggctttatgcgttccgtataggatattatgatgctggagttttgcgaagctccactcctttccgaacatttacacagatattgcctaagaaaagtgaaaccataatatggttatagtccctttcagtgcaaatacttagcttgttgcacagagaaatagaagctgcataagctctttcttcaaaaaatgtaaataaagttaaatataggaaaaattacttaatctatcttaataacggaaagattttgtatacaatcactgttacagaaatttcttagttactctgtaaacactaaatatagtttggtatattctgttgaatttatgatggatatgggacataaaaacaaaatagatataaaggttcgcttgcataactatattattttatatgcaaaaaggtgttcaatgacggttagctcaggcaatgaaactttgactatcgatgaagggcaaattgcttttatagagcgaaatatacaaataaacgtctccataaaaaaatctgatagcattaatccatttgagattataagccttgacagaaatttattattaagcattattagaataatggaaccaatttattcatttcaacactcctattctgaggagaaaagggggttaaacaaaaaaatattcctcctctctgaggaggaggtttctatcgatttgttcaaatctataaaagagatgcctttcggcaaaagaaagatctatagtttagcttgccttttatcagctgtttctgatgaggaagctttatatacttcgatatcgatagcttcttctcttagtttttctgatcagataaggaagattgttgaaaaaaacatcgagaagagatggcgtctttctgatatttcaaataacttgaatttatcagaaatagctgttagaaaacgattggagagtgaaaaattaacatttcaacaaatccttcttgatattcgcatgcatcatgcagcaaagcttttattgaatagtcaaagctatattaatgatgtatcaagacttatcggaatatcaagcccatcttattttataaggaaatttaatgaatattatggtataactccaaagaaattttacttatatcataaaaaattttaaatgcttcatagcccatcgctattgccagatgggttttccccacacctgatggccccagtaatacgatattttcattacgttcacgtataggaaattgaaaaactaagcgatacccctgtccgcgccttatgccacgtgagcctttctgtttttccccaccactccggtgttttctctctctgcatggtcgattcttataccgcaacaggctgttttttatacttgcaccgattcccgggcacacttagcctgtgtcaggccggcatatacctcagcagactgatatcccgcgcgtttttcttcagcccctccacattcagccccgcaatggcccggcggtataccatccggccgccacacagcacgcattcgaacgggtcacgactcaggaactgtttcaccatttgtgcatagcacactttcgccactggttccggtttatccatccccagagcacggtacacctgcggcagcttctctccacacacccggttggcaaggaacccgaagtacctcaccatcttaaaaaacttctccgggatgtgctgtttcagcctcgcgaccagctcacgctgtgtcagcgtttccgtcgccgtttctcccgttttgtggtccaggtaacggaagctcaggctcgcccctccgttgtaatgtgccagccgggaggccgctatcggcggcttcttcagataacgacccaggtagcgcgccgtattcctcccgccggccgtttttttcgacatgtacacatgccagtattttcctccggctttcagcaccaggcttctccactgtgattccgtcgtgatatgtgacaacgactccggcatcgccagcccctctgaccacgctttcagaagccgttgccgcatattccacatccaccgcgaacgcatcgcgtctttcaggaagctcagctttttccactgaccatgcttattcagacatccacaggttacagacacatgtacatgcggatgccagttgagacgacggccatacgtgtggatggcgcagaagataccgggttcctgcccccgtttccgggcggcatacagcagattctccaccgccagacggcacacgtcattcagcagccaccggttgctttcgaacaccggccacagcgtgtccggcagggtgaagaccagatgtacccagtcgcagtcaggaagacgattcagctgtgtcgctatccacaggtctgtggccttttttccgcaggacgggcaggcacggctgccgcatgagttagtgacgtatctgacgtgggggcactcaggtttatcgcagttgtattccttcactcccagtatccgcgtgccgcaggccagcatttttgtgacagcctcaacctcgatatcgcgcagaccgcccgcatccaggaaggacgtccagcactggttggccgtgaacagacgtttcagcgggcggggagtaaaagcggacaacatgaggacggattaacccccggtatcctgtagccgggtaagggctgctgtgtcgccacggcgctccaggatggaggccgcgaccatcgcaggtggtgggtccatgggtttgagcatcttcatccagtcccattcacgttgtgagagcttctcagcaccaacagagagcagaacggtatctatacttttgcgggtcaacataagaggatgagtccttaataaaacaggtcctcagagcatacatattctgcggagcgtgcggcaacaacaggacgaacaggggcggttttcaatttgcagccgtcaggctgccgtggttcagcgcctgatgggaatcctctcacttaaggcagcgattaaggtcaagcgataccgctcttacagaggagaggtagggcaaaccgcccccaatgttctccaaagagatttcaaggctacgcggccaaacgagaagtgggttaccgatgttactgaatttgcagtcaatgggcgcaagctgtatttgtctccagtaatagatctcttcaacaacgaagttatttcttacagcctttcggaaagaccagtgatgaacatggtcgagaatatgctcgatcaggcattcaaaaagcttaatgaaccgccccgggaatcctggagactaaacttcctgagaaagaggtaaacaggatgactaaaaatactcgtttttcccccgaagtccgtcaacgggcagtccgtatggttctggaaagtcagggcgaatatgactcacaatgggcgacaatttgttccattgctccaaagattggctgtacgccggagactctgcgtgtccgggttcgccagcatgagcgggataccgggggcggtgatggagggctcaccaccgctgaacgtcagcgtctgaaagagctggagcgtgaaaatcgtgaactgcgccgcagtaacgatatccttcgccaggcttccgcttattttgcgaaggcggagtttgaccgcctctggaaaaaatgatgccactgctggataagctgcgtgagcagtacggggtcggaccgctatgcagcgaactgcatattgccccgtcaacgtattaccactgtcagcaacagcgacatcatccggataaacgcagtgcccgtgcgcagcgcgatgactggctgaagaaagagatacagcgcgtatacgatgaaaatcacaaggtatacggtgtgcgtaaagtctggcgtcagttgttacgggaaggtatcagagtggccagatgcactgtggcacgtctcatggcggttatgggacttgccggtgttctccggggtaaaaaggtccgtacgaccatcagccggaaagccgttgccgcaggcgaccgcgtaaaccgtcagttcgtggcagaacgacctgaccagctgtgggtggctgattttacttacgtcagcacatggcagggcttcgtctatgtggcgttcatcattgatgtgtttgccggatacatcgtggggtggcgggtctcatcgtccatggaaacgacattcgtgctggatgcactggagcaggcgttatgggcccgtcgaccgtccggcacggtccatcacagtgataaaggttctcagtatgtatcgctggcctacacacagcggcttaaggaagccggattactggcatcaacaggaagtacaggcgactcgtatgacaacgcgatggcggagagcatcaatggtctttacaaagcgaaggtaatacaccgtaagagctggaaaaaccgtgcagaagtggaactggccacactcacgtgggtggactggtataacaatcgacgattgctggaaaggctgggccatactcctccggcagaagcagaaaaagcttattatgcttccatcggaaacgatgatctggcagcctgagttcacagataaaacactctccaggaaacccggggcggttcagttgctgaactgctggaaccgctgtatgacatcctgcgccagtatgtgctgatgcccggtaaagtccatgccgatgatatccccgttccggtccaggagccgggcagcggtaaaacaaggactgcccggctgtgggtctacgttcgtgatgaccgtaacgccggttcggaaattgcagcataagttatccatgctctgaggtgggtggaggtgggccctccggtcatgtcaacgccacgatgtttgaccgttatttgccattttcatgcccgctaaatactttcgttactgatgctgtccgctttccaccatttcatcatcactgattggcgttgtgtttttggtcagcactccagctttacgtttctcttttaaccggtaactctctcctttgatattcagcgtggttgagtgatgtagcagccgatccaggatcgctgttgccagtacgtgatctccgaacatttctccccagtctgcgaaccctttgtttgacgtcagtatgatgctcgctttttcatatcgacggttcagtagccggaagaacagactggcttcctctctgttcatcggcagatagcctatttcatccaggatcaacacccgggcataactcagttgctgtagctgacgctccagccgattttcctgtttcgctttcatcagtgtcgcgatcagtctgtccagtggcataaacagtacccgatgtcccgcatccaccgctttcacgccaagagctatggccagatgagtttttccgacaccaggagggcccagcaggatcacgttttcgctgcgctccacgaacgccagaccagccagttcccggacaaccttacggtcgatgcccggctggaaggtaaagtcgaactgctccagcgttttgacccacggcagacgagcctgcttcagcctggactccataccgcgctgatgcctgccgttccattcctgctgtagcgccatgcacaggaactcccggtagttcagctcttttttagctgcctgttccagcaggctttcaacgtgataactcagatgctccattttcaggcgactcagcaggacttccagttcatgcatcacaacagctcctcataggcactcagtggtcgatgttccacctgactgacctgctgccagagcggggcgtgatgctccggcactgtctgccagccagacgatgctgaacagaggcgatgtgaggccaccagtttctcattactgtagatccgcaactcgtcatccagcgatattcgtatcgataccggctgaccacacagcgcttcgggaacgctgtaacgattaccaccaacctcgatatagctgtcccaggacacatggcggatatcgaagtaactggtatcgaagtctgtatccggtaacggctgcagatgttcctgctccagcgcgaagcgctgttccggcgtttctttgaactggcgaagctcccgtttgtcagccacatcggctatccattgctccagttgttgattgacatgagtgaagctgtcgaacctgcggtaccggacgaagaagttctccttgaggtatttcaccatccgctcaaccttaccttttgttctggccctgcgtggacggcatgcccgtggcaggaagttatagtggtcagccagcaacaggaatccggagttgaacacgactttcccgttgttattcttcagcaccgcagccttctggttatcaaccagcaccgttttcacacaaccaccgaagtagcggaaggcgcgaaccagtgattcgtaggtatgctcagcatcctgttttggtgcggcgaagacatggaagctgcgggagaaccccagcgtattaaccgcaaagttaactttgcaccgttgcccggcaacctccacctcaacttcgccccagtcatgctggagctggtatccaggctgagtttcgaagcgaactgttctttttgacggacgcattttacgtttgggctggatgtagtaacgcagcatggaacgtccgcccgtataacccattgccttaatctccgcgaagataacctcgctattccagacattctctgccaggcgcatgtcgatgtaatccataaacggtttcagcttaaccattttgtggcgggtctttctggctggcggttcagggtatttgaggtagcgtctgaccgtccgttcagagcaaccaatctgagtcgcaatatcgacgatgtacgcgccctgctggcgcatttgctttatcatgtaaaagtcctctctgctcagcatgttgatgtcctttctggtgtgagaaccttaaggaaacaacatgttgggtggagcggacaatccaaatggcgaattaccgtcttatatcactggcgctgacagtcagggtttacaggagcactgaccaaacgacccggtgctgcttcattctaaggaatgaggtggtgagcgactcgggaaaaggcagagatctactgtcacgtggggttcgggaagctgaacgaaagtaaactgtccgatgaagcatcgaaatcttagatgatgtcaaaatggggccgtacagccgccccctgataagcgcagaagacacctgcttactgactgcgcggcatccggtgtgaaggcagcaggatcccgggccagaccatctcagggaacgtgagaacctgtttccggcacgtatcgggaaacaggggcggacccattcgtagtagtgatgaaagatctgtaatgggtctggagcgaagggatggggatcacattcattgcagaagacacaacccaaaaggggatgacgtaactgatgcaacgaaaatcatttgaaataccaaaagcactggtctgggcatcatatctggatgtgcgtcgcaacaagggagcccccggatgcgacgggcagactttgaaaatgttcgaccagcaacgtgatggcaatctatacaagatctggaaccggctatgttcgggaacatggtttccgccaccagtgctggaaaaacggatcccgaagtcgaatggcaaggaacgcattctggggatcccaacagtatccgatcgaattgctcagggagcgataaaacttttcatggaagaaaagcttgacccgattttccatgctgattcatatggttaccgaccaggcaaatcagctcatgacgctctgaaacaatgtgccatccggtgctggcgttatagctggatactggaagtggacatcagtgcgtttttcgaccatgtgagacatgatctggtgctcaaagctctggaacaccacgggatgcctaaatgggtcatcctgtactgtcgacgctggatggaagcgccaatgcagagttgtgaaaatggagaattaataacccgaacacgaggcactccgcagggcggggtcattagtccgctacttgctaacttgttccatcactatgcatttgatttgtggatggaaagagaatatcgggggtaccgtttgagaggtacgctgacgatattgtagtacattgttcacgaatgagtgatgcaacaaggctgaagaacaggttatcggagcgcttctcggaagtcgggctggtcctgaatgcagggaaaacgaatatcgcctacattgacacgtttaaaaggagaaacgtcgcaacgagtttcacctttctcggatatgacttcaaagtgcgtacgctgaagaatttcaaaggcgaacggtaccgaaaatgcatgccgggtgcgtcaaatgcagcaatgcgcaaaataacagaaacaatcaagaagtggcgtatacatcgctcaacagctgagagtttgctggattttgcgaggcgctacaatgcgatagtgagaggctggatcgagtactacggaaagttctggtccagaaatttcaactatcgactgtggagtgcaatgcagtcacgtctgctcaagtggatgcagtctaaatacagactttcgaaccggaaggctcagcgaaagctgacgctggtaaggaaggagtatccgaagctatttgttcactggtatttactacgtgcatcaaatgagtgatcaagagccgtatgacgcgagagtgtcacgtacggttccgtgagcagccggggagtgaaattctctccggctgactcgactcgcgggttccgacagcgatgacgaacatgcggcggtgttgtactcgctgatcggcacatgccgtctgaacaatgtggaaccagaaaaatggctgcgttacgtcattgagcatatccaggactggctggcaaaccgggtacgtgatctgttgccctggaaagttgatctgagctctcagtaaatatcaatacggttctgacgagccgcttacttttggcgtaaatctgctgcgcctttactgaacacaatgccgtattcagcaagaagtccacgaatgtgatttatcagttgtgtcctttccattgaaaccaaccgacgcatattcaaaaaagtctggacgtcctgttgctccagagtttttattggtacgggtcgtatagttccatgttggcaggcctcagctattgcctgtgcgtcgttataatcatttttctggcaccgctgataagctttaacatgttgtgccggaagcaatattacatcatggtcgagtttcctgatttcccgggcccagtaatgagatgccccgccagcctccatcgcaacagtcattttaggctgttgcaccagccagttaagcagttgattgcgagaaagtttggtctttcctgcgggtttgccatgagcattgatactgaaaagataaaagttcgttttagccagatcaatgccggtgattttctgttcagacatggtatcccgcttacttttgtggttaaagtttatccatcatggcgcaattgaaggctgccgtgaaagagggggaagactatctcatcggttactgttaaacggctaagagaaactatctttttcaagcaaagaagatacggtgaaacaggcgatttcaccgtgaaatactgttcaaaaaaaacgctaataaaagcaaaatttcttttaaaacagccaattaaaaactggtgagttgctggcgaaaaacttgaattttcacagcgaattcagattatcttttagtacaagagcaatcatacgatctaacttatcctgaacttccaacggtaatctgccgaattcatatgagaacgtacgtccctttactcgttttcgagcaaatttatctttactatcaaatttccataacaaagttacaaccgctttatcctttactcctgaatcaagcaatgaagacatttccttggttataagccgcataatactgtttttaacctcgtcttcagaaagattgtcatcagcagtaataagctcaatactcggcgagatattcttcacaacttcatcaatagatatatcacccagcctcagtcgttcacttaattcagccagttgcctgtaatcggcgtatgtcagctcagactgaacaggaaaaagagacacaaaatctttaggtacggacgctgcttggagggcacgagtcaccttagctgccgacaatccctctctttccgcaatttgcttctggttcataccggcctctttgagacgtactaaacgcaacccaatttcacgaatattatgttcaagtgacgtctgcaaatctttcgccagatgctgagcttcagaaacagtaagctcatcatccgttacaagtacacgtaatccaaccttgcatagcaaagctgctgcccgacgacgggaaccatccaaaatctcaataagatcgccagtccgaatcccaatacatggatagaattgttgtaaatgtatagttctggtgatgtcttttagtgagtccggcgtcagggctgtctgttcgcggccattcacctcttcaaccacaaaagtttgagtgtccactttgtcatgatcaaccttgatctcagtaaattttgcctttcggccagtattgagggtaaatacatgaactggagccgatatttcttcagtattattcaggatatttgtattcagcgtacggccaatagtcggtcggtgctttctgttttccatcagtagttggcccttataaattcaatacgatcaaaaactgctttagcgaaatcttctgctgccatacgtgcatttttcaaagcttcaccactgccaacataagtaacaggattcgcagatatcacggtatcaaacgactcgccgcttctctcaaaaccatcaagacgtggcattgaaacatcaagcatgtcaccaccgaaaatctcttttgtcaggctatggcagtatttatgatctgacttattcgccagtttcgacatgaagccaatatttgcctgcaaacggcatgaacatccatcctgttcaataatctgtacaagttcaggtaaacgagccaaatatttaagcgtggaatggaaatcaacttgtgctggcggcaccggtgtaaacatgatatctgcggcagcaatggcatttttcaaaaatgcgtccagatgcgggcctgtatcaataaggatgaagtcgaaatcatgcttcagcttatcaataatattctcacgcaatatcgcgtgtttgttttgtcctaatagatgctcctcgcacagagtatcccagtttgatgcaataaaggcgtcgtcaatagaagccggcataacataaacgccagggataactgacggaacgataaaatcttccagtagttcctcacgactcacgttctgcaacatagcctgcggtgcggttgtatcaaccagccccaccgcatgcaagtagttcagaaacatagtggctgatgactggggatctaaatcaaggagcagaatacgtaaatcttctgacagcaggtgaggatgagcacgcagggcgtgagctacggaaacactactcacggttttagacacaccgccttttagactaccgatgaatatggaatatgcctcactataacgatcgcgatatttaggaatgccacgatgtgcatagatatcgataatgttctggatggttaatgcatattttgtcgcggttcctgctgggcgtttttcaaactgatagccctgagcctccatctcaccgactgcaccatccacgcttgcgcgcgttaattttggaagcttagctacagcagactttgaatagacttgataaaaaacatcagcctgaaactcctgcttttgctcctgaatctgttttgttagggcgagcaacattttatctgcacgctgcgcaaccttacttaactgctcaaaactagtcattccccctccgtgctatgtaggattttaacttattacaaaaaaaactgcattagcagggtaatgcatgcaagtacatattgtacatgcttatattaaaaatgcgcgcattgctgatttattaatcatcggaatgaatattactgtaagatagtatgcctttccgggagaaagacgctgatttcccccgaataatccagttcaccaccacactccggacaacagctttcttcaggctccagatggtgtatttcgcgggaaacatgttccggtaacggacggcgatgacgtgactgtcgcaacaggcgggggtagtgcttccaatcagtagaacatgtgcttttcaatgaaggagccgatgactttttcgtggatttccacggaacgcgagaagcagattgttttacgtgccagccgcttgatgcgggtgcggagggtccggttgttacgttcaatacgttgcgtgaagattttgccggtcaggtgtttctccttcggcacttctctggcgtagctgccccagtcatcgctggtcaccatgccgatacagaacggagtcaacagggctaacagttcccgacaggtctcatcattccgtggcccgaaagtcgaaagtgtaagccaacacaccgccagttttagtgttgtacgcataccagagccagtgttgtgtcaacgacggatgaaaagtgatccacttatatctccaccaacggcccaatattgatccaccgttttactcaggattagcttctgctataaccccggcctttcgtttctgtctgagtcgatagctttctcctttgatttgaacgaaataatccgacggtgaatcacgctgatattcgcgggttggataccgcattacagaggtccgtcatatgagcctgccactttcttacgtcgcgaatcagctttgttccgccgtcgccaagcgctaacgtgcgttactcttgtaccgggctacgtcgccttcagggaactggttattctcattttttatttgtgacattgtggttgttgtgcgacagacattgctattcggcccatatcccagataaaacccgccagctcacgtgcaacagcaacaattgtaatattgacattctttcctttggcctgaagttttcgatacctcttacaaagcctgagttgagcatcccatgctctgtcaatgacggggcggggtaaattttcctgccttttttgtattgccggactgatgcgtgccggatgacggtaactccacgctgcctcgaccaatagctttctggcatagctgtttccggctttggtaatgctgccctgatgtctgcttccacccgatgaatattctgatggcgtgataccaaaccatgacatcagttgtcgtggatgttcaaaacgggtcagatcacccagttcagaaattaatccaaccgcagtaataaactgaatccctcgcatggcttgcagcgcctctactaccggatacagcctccattcggtaacggcctccttcagggcggattccagccgttcacattgtgcctgccgatcttctattgtacgacggtgttcgtcaaaagctaattgtcgccagggactttcgaatgagtatttgctgagccagcgtcggtgtgctgggccccaatctgctcgcccgacataatggaccccatgaaccagaagaaacgatttcagacgttgccttgcatgccgtaaatcatcgcgggcagatgcccatgcccgggccaaatcccggaatgcctcatcttcaatgccgggtacgtagacagcagaaagatctcctgcacgcagtgaacgcacaagtctgatggcatcacggcgatcggttttaacccgctcccccggttttttcgggataagtgagggggcgcagaccatgcagtcaaaaccggatttcaccagccgacgataaagtccataaccacagggccccgcttcataaacgatactgacctgcgatgactttgacctgagacgtttacaaagattctgaatatcagtaggtgatgtgccaattttacccatcagttcaacaggttctgaattgatagcataagcgacagtaatcgattctttgtgaacatctaacccgacgtacaatgtgttattgttgttcatgctggcctccgtggtggaaataccggggtgtagcaacagctcacccatgcggctctgacaacgatgttaacccgcgttaaatatcccacgttggccagcatctgtctcacggaaagtcatactgccttataaggctttccagttgcaactgcccggcgagcgccatcagtcgttgatgttgcagttccatcatcacgccactcctctgcagaatgagtcgtagatggagagtggatgatgctggggggtgcccctattaagttgtcaagcatgttatgacccctgcggggtataaaaagtcccgtcgcgcatcatggcgaacagaacgtcgcagcgtcgtctcgccagggcgataagcgcctgattgtgtcgttttccctgactcattttgcgggtgtagtaagccctggagagcggatccctgagcgcggcgaaggccgacaggaacaacgcccgtttgagagctttattaccccgtcgcgagggatgctcaccgcgtattgacgagccggatcgccgagttaccggcgcaaggccagcataagcagcgagatgtgcggcagaggcgaaggcgcggcaggcgacctcggtgaggagtctggctgcggtcctgacaccgactccgggcatactggtcaggaccgggtaaagagggtgagcaagaactcgctgttctacctcaagcgccacctcgtctctttgcttacgcagcgtgatgagctggagtgccagacgtggcagtactacggcagcggcattcgtgccgggaacgacgacggtttgttcggccagtgcctgagctatgtctgctgcaaggcgtttacccagacgaggcgcaagtttgcagagctgggctgccagcttcttctcacccagcgaagcccagcgaagcgagtttttctggtgagggatatcgctggagaagatcgagtaccgccgggtgctcaagtctcggaccgagaacgcgctccagtgccggatgtatctgggtcagaaggccgcggatacggttgctggcctgcgttgtctgtgcggcaagatcatcatcgaagccgcagagcatggagagttcggcgatttgctcgtcagccagtttcagcgtgcgtagcgcgtgaggcagggtacgggcagcttcggcaatgatggcagcgtcacgagcatcagttttagcttcaccggcgtgtaagtcggctatgcggcgcatggccagtccagggaggtatccgacaaggactccttctgagcgggcaacggcgacaggtaacgcaccgatggtagctggctgatcaacaaccagcagtatctgaccatgttgtttcaggtcagatattagcgacctgagtttgttttcgtcgttgggtaatgctttatcgaacaggcgtttacctgaacgattaatggcaacagcgtgatgcgtatctttaccgacatcaacgccgataaagacctggacggattcgtaatcgctggattcggtcattctgtctcccttgtatatgggttaaccagataaccacggggagcaggtaccggcatccacgttacagacggtcccggcaaaagtgcctgacctgacccctattagcggttaccagcgccccaccagacccggtgacatcaccccccggatcatggacgactgggggcagtaatcatgccgggtctggctggctaacaccccattataaggggtacgaataaagtaacggggttgtcgaagctcaccagattttcaccaggatgcacgtcatactcttttttctccggaggcagtgccagcatggactgctgctcttcgagccagcgatcacagggacgggcctggattgtttcatgctttcgttggttagcgacatcgtgcagccagcgcagaccgtggcggttggctgtttcaacatcgacagtgctccccatcgggcgcaggcgagtcattagtgggatgtaaaaactgttacgggtgtactgcaccatccgttccaccttacctttagtctgtgccctgaaggggcgacacagtcggggagagaagcccatctccttgccgaactgccacagcgaaggatggaaccggtgctgaccggtctgatatgcgtcacgttgcagaaccacagttttcatattgtcatacaacacttcgcgcggcacaccaccaaagaagcggaacgcattacgatggcaggtctccagcgtgtcataacgcatattgtcagtgaattcgatgtacaacattcggctgtatccgggaacagcaacgaacacgtgaagcggtgagcgaccattacgcatagtgccccagtcaacctgcatctgtcgtccgggttcagtttcgaaccgaacggcaggctcctgctcctgaggaaccgagagagaacgaatgaatgccctgagaatggtcattccgccacgatatccctggtctctgatctcgcgagcgattaccgttgccgggattttgtaaggatgagcatcggcgatgcgttgacgaatataatcccggtattcatccaggagtgaagcaacagcaggtcgcggcgtatattttggcggctcagattttgcctgcaaataacgtttaacggtattgcgggagagccccagttctctggcaatcgcccggctactcattccctgcttgtgcaggattttaatttccataactgtctcaaaagtgaccataagctctcctgaatcaggagagcagattaccccctggatctgatttcaggcgttgggtgtggatcactattgcaccgttcgtgacagtgttgttgcgccttgctgccaacgaagctccactgcccatcaagttcacagataagggcaacatcagcgtgagcgactggcgcagatgttatccgccggggcgggagctttttaatgtccgtatgacggtattgatgccgattttcagcgtacgggcagtatcgcgaactccggcaccgttgaaagccatttcagtgatttggtttttaactcccggtttacgggcctcgtaacagtaagtgagctgaaacacccggtgacagtcacggcaacggaatcgatcatgtccttttgggttctgcccgtgacggtaaacctgaacggactggcaacgggggcaatgaaggttaactgtgaccatgaaagaaccttaaaagcgggtattataacccctaattcaactaattggaggcattgtcatttttaatctatggatacacttaaaaatcctctcaccttacgttaaaaaccttctcatgtttagagttttttattatgtttatcataatgatatgacgatctgagtttttaaaatcaagaagaaaagataagttttttaaatgcacagtgcctgtggataactttgcgtaaatccggaggacagatcactctgaagcaagacaacagcatctgaaaacaatatgctcacgtttcaaccacagggcgtaagtgatcctacccacgtaatatggacacaggcctaagcgaggttctggttttcaaattgttccggactgaggccgccacaccaactgtgccgtcgccagcgattgtaatcacattcgatataattaaacactgttgcccgcattatttcccggctgataaagtgttctccgtggagacattccactttcagcgaatgaaagaagctttccacgcaggcattatcgtagcagcagccctttgcgctcatactgccacgcagattatgccacttcagcagcgcctgataatctgctgaacagtactgacttcctcggtccgagtgaacgataacgttccggggtctcttacgccgccacagcgccatctgcagggcatcgcaggccagttgcgccgtcaggcgtggtgacatggaccagccaataacggcacgtgaccacaggtcaatgaccacagccagatacagccagccttcatctgtacgtaagtacgtgatgtctcccgcccacttctggttcggaccactggcgtaaaaatcctgctccagcagattttctgacacaggcaggccatgtgcgcggtaactgaccgggctgaacttccgggaggcctttgccctcagcccctgacggcgcaggcttgccgccacggtttttacgttaaagtggtaaccctgagcacgcagttcatccgtcaggcgtggtgcaccgtaacgctgttttgaccgggtaaaggccgccgcgaggacaacgctgtcgcagtgctgacggaactgctgacgagggcttatccctgtccgccgctgacaccacgtataccagccgctgcgggccacccggagcacgcggcacattgccttgatgctgaactcagcctgatgtttttcaataaagacatacttcatttcaggcgcttcgcaaagtatgtcgcggccttttggaggatagccagttcctctgcctgctccgccagttgccgcttcaggcgggcattttcagcggccagttcactttcacgctcagacgaggtcatctgttgctgcagtttgctgcgccacgcatagagctgagattcatacaggctgagttcacgggctgcggcagcaacaccgatgcgttcagcaagcttcagggcttcactgcgaaattcaggcgaatgctgtttacgtgtttttttgctggttgatactgtttttgtcatgtgagtcacctctgactgagagtttactcacttagccgcgtgtccactattgctgggtaagatcaatttgctcactggtatttactacgtgcatcgaatgagtgatcaagagccgtatgacgcgagagtgtcacgtacggttccgtgagcagctggagagtgaaactctctccggctgactcgactcgcaggttccgacagcggtgacgaacatgcagcggtgttgttctcgctgatcggcacacgccgtctgaacaatgtggagccagaaaaatggctgcgttacgttattgaacatatccaggactgaccggcaaaccgggtacgcgatctgttgccttggaaagttgatctgacttctcagtaaatatcaatatgattctgacgagccgcttacctttgagcagagaaaggggagagggatcccacatccatgagctaggaaaatacaaaaactggctatacaacaattcaatatattaggacttacttgcgaggagaaatatagcaccgctcaggcaattcctgggtatttacttttcttagtttctttactcgggatgttctgtcatcagaaaggcgattctaaatttgtaatatagtcactcatattcccacgtaatttctttagatcaatatattgcacatttttctcaaaaaactgatcggaaataagatttgcaacattgcttgttgtatcatttttaacgcttctattcatcccttcacaaagttcgtccattttctttttgaaataactgcttcctgatacaagatgatttatggaactttgtgcagcattgatgtctagagaaatgcgataatctttctcaacctgtttaaatatctgtttacggaggttatcgccgatttctcggctgtgttctgcacaaaacttatcaatacattggtttaccacccgggatacgtagacttttttttcatcatttttccaatctgtaatattttcaccgatttgttctgaaacagagctcaaaatactagtatttattttggttgatgatatttttactatccttttccccattgtacagcggataatctcatccacctttcctgaaaaatatcttttgtgagataaactgattccaaaattattaaaagatgttccaagcattataatcactttgtcatgtaaaaatagtaacattgtcattattttagctgattcaaacaattctgtaaatgctttgagttctttactcccttggctcaatgatggaccgtctggaatcccagagactaaactctctgagaaagagagtatgaaaaaacttggggcagtccctaaatgaagggaccaacagcggtaaaataaccggcatcattcataatgatgccctgttagttgtagttatgaagatttacgtcattaaaattgctgttcgtagtgtcagtcctatggtctggcatcagctgaggatcgtagccgatacatcactggcttcacgacattcgtgttgaggctattcatgaaaactccacgctgaaaacgccgttttgtataaacggtcacggtatgcccggagccactgttgctgatgaattcgataaaaccttggcgtttcttgaagctatcgttaatgcggataatgaaacaacgattggtgagattcgctcttttgccgacgccctggacgctgtccggttcaatcgcaacaaaatcaaccggcagctgagcaaacccaaccttgcctctctggcgctggagcatgaagtgatttggctgggacgtagccgctgacagcttctataaaagtatgatcctacccacgtaatatgaacacaggcttaagtgaggttctggttttcaaattgttcaggactgagacctccacaccaactgtgccgtcgtcagcgattgtaatcgcacttgatataattaaacactgtatatttcatttgtggagtctccagtctgagggggctttatgcattccgtattgggcactatgatgccagaaatctgcgaggctccacaccttcctgtatttcccttcctctatcacagtgcactttacttaatagggtgggatgtgttcattacatttattgtgttttttatgattgtttttgcgtccagtgtcgttcgtcatgggaaactggttctatgggtagtatcgctcagtctagttaactgtttgtttctttttagaaattaagcagatcacacaattcggggagagatccttctaagtgcaattctttgcagctatttagcacacttaaaacaactcttgtattttgtcagaaataatattaaacatgtcttgttcattattgttaagtctgtctgctatttctgtaagttttatcccactatgatggcatatatttatatatgccatcatagcctcgatattccctttccctgcggccagaaataatcctggtaaaccctcattatctcttacatccaatagtttaatcgtttcttttggtgataaatcaaaagttttaataaaatctccataagcttgaatcgcagcggaatgaccttcttgaagagctatgaacaaagcagattccccagcattatttcgtgctgcaagaacttctagtttttcctctttgggtatttcggctgccaacagcgattcaccgtatttacgcgctgcatcggcatttccatgccaaatcaatgaaaacaaattatggcggttatactccttattattctttgtcggaatggatatgctctgtattttggccaattttctgaaagaaacaccactatatcccccccctgatttttcttcattgatggcgtgggatatagcggaatttgtcacgttaatgatattacttttcattcctttattttcattagcagtattgctgtctattattaggtgcatatttttgttgtttgctggatgtaatccatagttatttattgacatttttgttcatcctttttgctaaaaaaaacaataacctatttttgtaatgtaatagatattagtctattgccaatctaaaaaaaccattgccacattgaatggctatattcaccaataaattctttcgatttcgtatattgacatatcgtagatatatcttatccctaagattcaataatcgagtccgtttaccattctgtgcaagcgccttcactcagaagtgaccgtcaaggcgatcaccaaacttgataataaagcagctcattgccatacgccagtccttaaggggcgttgtccatttctgtgatgctgcctagatttaactgccagctccggcttatctacatctaaatatgtgagcctaataccgctacactttggccagactatgtttttctccgggaagtgggctaaccgtctccataaagtgacgaacttttttcaacagtggccatattgaacggcactgatgattacgggttattgtctcatgaagagccggccacatcactgacagcagtgacttcaggccttcagcatcagacagcctgaaccagttaatccatcgcccgacagatttagtaataaaggggcttggcagtttggataaactattgttcttattccactaatacaaactttttgtaaagtatattaaatatacttcatttcatactgaaatccccggaagtattcaatcaaacaattacaccataaaaggaagagggggccctaattaagttgtcaagcatgtttatgatacctgcagggtataaattttctggctgcaagcagacacatataaaacgtctttattgatcctacccacataatatggacacagtcgtaagtgaggctctggttttcaaattgttccggactgagaccaccacaccaacggtgccgtcgccagcgattgtaatcgcactcgatataactaaacactgttgcccgcactatttcccgatggataatagtgttctccgtggagacattccattttcagcgaatgaattttcatctgttgctgtatccagtcatacagtgactgcatcagtggcgcggctctggcttttcttgccgccagacgctgttctgctgaacatccccggacttctgcctcgatggcatacagttcaccgatacgctgctgggcttccgtggtgatgtcggttggcactcttgcatgcacatcgtggatttttctccggacatgagccatacatgcggcttccgttattctgccggattcgtacaacgcccggtaactaccgtaagcatcggcctgaagcactccgctgtaaccggccagatgattctgtggatggatacctttccggtccggtgagtacgcgaaccagaccgccggaggcatttccgaaccggcgttacggtcatcacgaacgtagacccacagccgggcagtccttgttttaccgctgcccggctcctggaccggaacggggatatcatcggcatggactttaccgggcatcagcacatactggcgcaggatgtcatacagcggttccagcagttcagcaacagcacctgtccagcgccccagtgtggcacggctcagctccacgccctgacgacggtatatttctgactggcggtataacggcagatagtctgcatattttccagcgacaacatgggccagaagccccgctccggcataactgcgtgcaatgggttttgaaggcaccggtgcctgcacgatatggtcacaccggcaacaggccagtttcggacgttgtgtttcgataaccttaaaggcgctgctgataagctccagctgctctgacacatcacatcccagagaactgagttcgccaccacaggccgggcagcattcctcttccggacggatgacccgggtttcacggggaagtgaggcaggtaacggtttatgggctgaagactggcgcaggggagatggcagtaccgggtcatattgctcacccagcgtttccgccatttcttcctgaagtgcgctgattcgctcctgtgcttcctgtatctgccgttcggtttttgcgcgaagtttttctgagcttttaccgaactgcatgcgctgcagtttcgcaaccagcgccttcagccggttgatttcggaagcataagccgccacccgctgtgagagcaggcggttgtatttcgccatctggcggatggtgtcctgttgcgtctgcaacagtgcccgcaggcgggcattctcatgagcaagtgaggtgtccatatcctcactttacaacgggttatatgcggatgccagcgcgttccgttcgtttcgggtgtttccagtcgatgccctcaagaagcatggataactgggccggagtaaggtgcaccttgccgtcacgggtgaccggccagacgaagcggccccgctccaggcgtctggtaaagaggcacagtccgtcactgtcagcccacaacacttttatctggtcaccccggcgtccgcggaagatgaacagatgcccggagaacgggtcatccttcaggacgttctgaacttttgaggccaggccgttaaagccatttcgcatatcggtgatacctgcaaccagccagatacgcgaacctgcagggaaagatatcatcagtggctgctcccttttatttcgcggataagtatctgtaataacgccggagtcagtttacctttaagcctgaggaaagggagcgcaatatgaagagctcagaaaactgggtaaaggcgatcatctggtgaagctgaaaaccagcccgcaggcacgaaaaaagtggccgggactgggaaatgaagtgacagcccgcctgctgaccgtgacgcgcaaaggaaaagtctgccatctgctgacgtcgatgacggacgccatgcgcttccccggaggagaaatggcggatctgtacagtcatcgctgggaaatcgaactgggatacagggagataaaacagacgatgcaactgagcaggctgacgctgagaagtaaaaagccggagcttgtggagcaagagctgtggggtgtcttactggcttataatctggtgagatatcagatgattaaaatggcggaacatctgaaaggttactggccgaatcaactgagtttctcagaatcatgcggaatggtgatgagaatgctgatgacattgcagggcgcttcaccgggacgtataccggagctgatgcgcgatcttgcaagtatgggacaacttgtgaaattaccgacgagaagggaaagggccttcccgagagtggtaaaggagaggccctggaaataccccacagccccgaaaaagagccagtcagttgcttaactgactggcattaaggccgccggggacgtttttttaccaccgggagtttgggcataacggtgctttcagtaagtacgacaggtactgtggaggcacttccgtaaagaagggcatcaagttcctgctccgacatacctgcgggcagaggccatgaaaggccagcgttgcggaagcgcacgaacataccacagacagttgattttggtacgcccaggcgacgtccggccacaacccgtggtaaatgttcttcgaaatgaaggcgtaaagcttcagtgatccaggtccggtattccatacgatagtgtccacttcaaaatgatggacattatttttgtggagccggaggaaacagaccagacggtttaaatgagccggttacgaagaagctgaggttctgcgctttgataattttgacgatgccgttgagtgttgcgcagacctgaatattccgttctttgtggatgccggaaacaaaaaactggtcttctggtttgtacgtgttgatgacgaagggtatccggaaattgcccgctgcatggagcgggaatttgcgacaattcctgccggtatcagcgccgacggcatgtattgccctgaatgcggtacggttcactggccggatggcgttattccgcctttctgatgcttccccgttttgccgatatttttcagcagggaaaccgatggcttaactggctggagaagcagtcggtgcagatgagccgcctggaacattatgccggtcaggatgaaattggcctgcgttacaacagccaccggacaaaacgagaagaaaaccttgtgatgagtggcgacgagtttatggaaaggttctcctggcatgtggcggataaggggttccggatggtgatacggggtcctgagtccggtgaagcggcgattactggaagatgtggtgtacgccataacggagacagtgagaaaaacggcgaggcaaatcacaaggagagggatgtatcagcagtgactgaaggttgacccgctgaagtgcgagctgtgcggaggtcagatgcggtttacggggctgaagaggggttaccgtctggcagagcaggttatgatgcatgagccgctggctcgaatacggtggtgcggctgagagtcgcagaggggaagttgcgtccatgttgaggggaacggagcaaaaaaccatcagagatacattgaatcaatcaatgtaagcaatattaagtgtctataccgattacttgatgatcaggttgtgttgaataaacgcgcattcttcggtgcttttttaatcttttggggatttaaattcctatcgatgaactgtcgatatgaaaaagcgagcatcatactgacgtcaaacaaaggggtcgcagactggggagaaatgttcggagatcacgtgctggcaacagcgatcctagatcggctgctacacgctgaatatcaaaggagagagttaccggttaaaagagaaacgtaaagctggagtgctgaccaaaaacacaacgccaatcagtgatgatgaaatggtggaaaacggacagcatcagtaacgaaagtatcttagcaggcatgaaaatcgtccgcatcccctaaacggtcaaaatgcttctgccgtgaaggagggagaatgccatctcatcacttatatctggcgaatgaaacacagatggtcggccttgcccccaaacacacggcggtgcaaaatggaatggcagaaaacttcgtcaaaacgatgaaatatgattaaatcagtgttatgttcgaacctgacgtgttaacagcagtaaagtagtctcgcgaaggtgttcaaacattataatgaatggcatcctcatagtgcgcagggttatcgcttgctgcggaaatatctgaagcgacgtaccagtaatggggaaaatgatagctggtttctgaaaatatagaggaaaatgtgatatatatatcctgttaatatttaaggagaatataaaataaaatgaaagtctcgtttaagtctttaggctatatttttcatgatatatataataaaaaacacactatcgatgagttcaacgatgtggtaagaaaagcagttttatcaggtaaaattaacgagctgaatgcatgtcataaagtggcaatttttcttgccgaaaaagacaatgagataacaaagaaagacaaagccaagataatagatacattaactgaaaactattccattgaatttcaacaattaatgaatattagtgagcgcacgttaaattcatctttatatattactcctggtgaaagtggttttgtcagttttgttaatcgtgaaggaaaaatatgtcatactgcatatgtaaaatcatcagataatagcatggcatattaccatgcaaattatagcagcattgacaaatatattacggatatgtgcggccttatttgtatgaggcatattgagtctactggcattattttttatatgcttgatgaaaaagttttatctgccattgctgaatttatgaatgaaaaaggatggcgggctgcgttttgttcagcaaagaacttatataaatgtgtttaattttagaaaaactatttggtaaaatccaacaagagtcgagaggttcgcgttgcttcagcagggcctcagcctgttcaaccagactcagttgcaggtcgtgacgcagatttagtggtggtggttgattgtcagataccagccgatgatcttgtagtgcatatcatcagactttgagaagcatatggttttgcgggtaagccgcttaagatgtgtccgcaggttcagattatgtcgctctatccgctgagtgcatttcttgcttataacatgactggttgaggacaataaagttcggtataccggctcaggcatcggtcatgtagaaggcaatgctaaactggctcagcaaactaagagcagcgcgcatccacttacccaatgcggttgagaaaatagccttcgatcgctttcatgtggcgaaacaaccgggtgaggtggtggataaaacccgccagaatgaacatccgcatcttcctgttgaaagtcgtcgtcaggcaaagggaactcgcttcctgtggcagcacagcaataaatggatgaccgaatccaggcaggaaaagctgatatggctgcgtgcgcagatgaagctgacgagcctgtgctgggcgctgaaagaactggcaaaggatatctggagcagaccatggagcgaggaaagacgcaacgactggcagagatggctgtcgctggcggccaacagtgacgtccccatgatgaagaatgtggctaaaacgatagggaaaaggttgtacggtatcctgaacgccatgcgccatggcgtatcaaacggaaatgcggaagcactgaacagcaagatcaggctgctgagaataaaagccaagggctaccgcaaccgggagcgctttaagctgggagtgatgttccactatgggaaactgaatatggcgttctaagccttcccaccatgatcggagaagacccttacgcttacatacattacatataatgtatcgttttataaaagatgatagacaatcaatataagataatatatctattttatagaggacgttttctatgcccataaaaaagccctgtctaaaactaaatttagattctttgaatgttgtgaggtctgaaataccgcagatgctttctgcgaatgaaagattgaaaaataacttcaatatattgtacaaccaaatacggcaatacccagcgtactatttcaaagtagcatcgaacgtgccaaattactctgatatatgtcaatttttttctgtcatgtaccaaggattccagattgtgaaccatagtggagatgtgtttattcatgcatgtcgtgaaaatcctcagagtaagggggattttgttggggacaagtttcatattagtatagctagggagcaagttcctctggcattccaaattttgtctggtttattattttcagaggacagtcctatagataaatggaagataactgatatgaatcgagtttctcagcaatctcgtgtggggataggagctcagtttacgctatatgtaaaatcagatcaggaatgctcgcaatatagtgctttattacttcataaaatacgacaatttataatgtgtctcgagtctaatctattaagaagcaaaatcgctcccggggagtatccggcgtcagatgttagacctgaagactggaaatatgtcagctatcgtaatgaactacgaagcgatcgagatggaagtgaaaggcaagagcaaatgttacgagaggaaccattttatcgtttgatgatagagtagagaagatgccagctacaccaccttgtagcttgaaaactggttagaataactacgattagatttggcactgcgtatgcagcctgattcctgcccgtgacagagcattcggctcataaactgctgagctactcatctgaaatccgaacagttgtccaccacttcagcgtgcatctcctgtttgagtcgtttgattgcttttcgggatattaacctgtcggattttgcattagttaatgctcccatcaggccttcatcttttccagtgcctattttcctgttctccttttgcacaatataatcagatcatctgtatcgttgacgagctcgctatcttataggagcattctgaataagctacctccttgtaaggaggcttttactattagtaactgcaatctaatagtattataacatctctatggttgtcttctatgccgttaaataaaacattttcatctagtatattttcaacaaaaaattcgttatcaacagatatgtctgttaacagagataatagaacaataacaagttctattatgagggtctcaaactccagtgaactaattcagtttaagaataaaacagcaccttatttttcagaaaagagaaatgtgaaggtaaatataaatggagtggcaaaagatatatatggaagacaaatagtttgccgtcacctagcttcctattgggaaatgaattttatggaaacaaatgggaaggtaaactatcagcttcttagcacccctgatgctattgcaaaaaatgtatgtttggaaaaaacggaagatttttcaaaatcaccagcttatatttattttgtggaaaataaaaagtggggaacagttattactaattttttttataacatgaaaaaaaatggagactttatcagaacattatcagcatgcacattaaaccaccaaatggcgttagggttgaaaataaagcgcgtacaagaaagtgaaaaatgggttgttcagtttttcgatcctaatcgaactgttactcataaacgtactgtatttacttgtgatagccatttcgaactgagtcaattatccgctaaggatttttttgatgatttttattggaaaatttatggtctggagcaacccgggcaggtaatatttgaagataggcataattcccctctaacaaacacagtgaaacttttaccagatgaattgatcaattccagagttatatatcatgcgataacaaagaatctgacagaagtattatttatactgatggaaaaatacaaaaatggtgaaatttctcaatctaaactggtgaaccttctggccacgagaagctctgatggtactcctgcattttatatagcacttcaaaacggatgttcagacattattcaagtatatggtaagatattaaatatgtgcaatcttagtcaagaaacaatacttacattgcttgctgcggtaggggcaaacaatgtgccgggtttatgcatgtcttttatgaacggacatgtagacacaataaaagcctatggggaaatagtctttaaaaccccgttaacttccgataagagattatatctattagctgcgaaagacagccatgatttacctgggttattttttgcattgcagaatggacatgcagatagtattcgtatgttcggaagtctgcttaataagaaaatgcttagtagtgaacaaattaaagaattattgaaagtaaagcatgggttgtttatggcacttcaaaatgggcacacaaaggccattatggcgtatggagatatactaaaaatactcccccctcaccaggaatatatagatgaattattatggataaaaaatcccaacggtacatcaggactttttatggcattttataatggccataccgaaacaatacgtgccttttgtaatattctaaaaaactatagttttaccacaagacgattggtcgaaatgctgtccgcaactaataaagatggtattcctggtgtatttgtatctgtagtaaatagagacaaagaaacaatattagagtattgtagaataataaaagagaacaatctggaaccggatactatagcagaacaattttcaaagaagatgaaaaaaacgtttattgaaataataaatagatttaatcattttttatgagactgtaatttaaattgtgtaattacctgtttttgatatgttcactccaacaaggtgtagtgaccgggtaactgccaactttcaggcaaccacctcgattagcaaccttatttccatatcgagttcaaatatcccgtaggggtttacatgggcgtaaactgatgattgttcaggtttccatggtgtggccaaatcttcttcatggataaaggtcacatccagagggtgagtatgttccaatatttaatactaaagatatgaggcttggaattgggttgcatcttattgattttataagaaagagtaaagatcaaggattccgagagttttgttataacaaaaacattgatcctgttagtctggatagaattataaactttgtgtttcagcttgagtatcatataccaagaatgctaagtacagataacttcaaaaaaaataaaactcagagacatatctttggaggatggatttgcccctatatttccagacatctgttatcacttaacccattacaagcccgctgccgcagatattcccgtggcgagcgataacccagcgcactatgcggatgccattcgttataatgctcgaacgcctctgcaaggttctttgctgccgttaacccgtctggtttgggcatgatactgatgtagtcacgctttatcgttttcacgaagctctctgctattccgttactctccggactccgcaccgccgtgttcttcggttcaagtcccaacatccgggcgaactggcgtgtttcattagcccggtagcatgaaccattatccgtcagccactccactggagacgacggaagatcgttgccgaagcggcgttccaccgctcccagcatgacgtcctgtactgtttcactgttgaagccgccggtagtcaccgcccagtgcagtgcctcacgatcacagcagtccagcgcgaacgtgacacgcagtctctctccgttatcacagcagaactcgaacccgtcagagcaccatcgctgattgctttctttcacggccactctgcctgtatgtgcccgtttcgatggcggtacagcaggttttcgctcaagcaacagcgcattctggcgcatgatccggtaaacacgtttggcattgatcgcaggcataccatcaagttctgcctgtctgcgaagcaacgcccatacccgacgataaccatacgtgggcagctctccgataacatggtgtatacggagaagcacatccgtatcatcagtgtgacgactgcggcggccatccatccagtcatcggttcgtctgagaatgacgtgcaactgcgcacgcgacacccggagacaacggctgactaagcttactccccatccccgggcaataagggcgcgtgcgctatccacttttttgcccgtccatattcaacggcttctttgaggagttcattttccatcgttttcttgccgagcaggcgctggagttctttaatctgcttcatggcggcagcaagttcagaggcaggaacaacctgttctccggcggccacagcagtaagacttccttcctggtattgcttacgccagagaaataactggctggctgctacaccatgttgccgggcaacgagggagaccgtcatccccggttcaaagctctgctgaacaattgcgatcttttcctgtgtggtacgccgtctgcgtttctccggccctaagacatcaatcatctgtactccaatgactagtctaaaaactagtattaagactatcacttatttaagtgatactggttgtctggagattcagggggccagtctagaggatgcgattaaagcatctaattatgaagagattaacaataaagtgactgataaaaaaatggctcatcaggcccttgcttattctttaggcaataaaaaagctgatatagcactttatttactatctaaatttaattttacaaaacaagatgtcgcagagatggagaaaatgaacaacaatagatattgtaatctgtatgatgttgaatacttacttagtaaagatggtgccaactataaagtgctagaatattttatcaataatggattggttgatgtaaacaaaaaatttcaaaaagcaaatagtggggacactatgcttgacaatgcaatgaaaagcaaagattcaaaaatgattgattttttattaaaaaatggagcggtatcaggcaaacgatttgagaggtgacctaaaatatatttaggttgtgccctgaatctgaagttgtcatttctgttatattgatcgacataggggatggtgcagaccgttcccctgcaacaccacattgcatgtggtgaacggaaccgccccgagtttcctagagaatgttttatctgtgaactcaggttgccagatcatcgtttccgatggcagcataataagctttttctgcttctgccggagggatatgtcccagcctttccagtaatcccttaaattcaggggtaaaacgtggtgtgcccatagactcctcctatgctcatatagggtagatttgtctatgggccctccatagcctgatacaggctgtccagctttatggggtcagttcattggtaggagtatttaattcttcaaactcttttttggcttcgctaaggcttttggtaaattcaggtgtattatgaagtactgcaacaaccgatgctcccattaaacgcccagcctctacatcgctttgccaatgcgcaccgcagatgacccgactttctccaaactcatatccacgtcgaagtatttccgctttacgttgaggattaatctccgcaagtatcagtgctactgcccaaccaaaggatgcatgaccagagggataagagccagtgatagccattttctcatctttatcaggtgtacaggttgcgtctttataaataacgaatggacgaactcttttataatattctttggcgttacgcacggcgtaatcatggctatcctgtagtacttgtgttaacaactgatataagattggggtattttcccttgaaatatccatgccataagcatctgaaaatgcaagaccaaaattctcgtagtatgcatcttcgctagcgagacgtacacgattagcatcccggagcgagcggccgaattcataatgagctttgtcagcctgaaatactactgaatcctctgccggaggcggcggaagtattgacaaactgtctggtgaagtttgttgagtgagaaaaccttctgccttcagagcatttgctgaggggataaaaatcatatttgtagcaatacaaaaaagaagaaagtttttggttttcataatttatttccttagcataattaattgatgattttggtcagctgtaaaaaacgagctgtgttacaaaaaacacaagtcatgcggaaaaatttgaacagagtgatggaaaaacaaaaaatagtcttatattaaagtaataacattatttttcatattcacgaaaaataatgtggtaaaagaatgatgttttttatctatttgttcgtatcgttttgtctctgtattatgatgatttgtgattttccctcatagggaagcacttttctactaaagataaaataacttcatcttaaatgcaaacaattaactataggttaaaaattttatactaatccagttctttattaataataaaagtctttttctgagagcttctttttactgtatgctccggatcagcaggaattgaagttgaacgataatgtgagtagggaaaaagctcttgaccatagtgaactccatatccatgatagccggatatttttagaccatcactcaatgactcatcgttttcaaggtgtttttttatctgctctagcaaagattctttttccttaaaaaaaggcagagagttaaggatacaagaaagactttcagcaggggcattgttaaaatttttaggagccactttatcagcggagccgcatgaagtaaaacggatatctttcacatttagaataccacagtctttcattcttgaaatgattgtttgtacatcaataagttccgaaccaaggcctaattgatgagaaccaggactaccgtgcccaagaatataaaccttattatactctttaaaatcagtatcgtttactgtttttattttcgctttttttacagaaaaataagcagcagcattcaatgcgtcatcatcattatttattactattggttcactctgtggtagccatgattcagccatttttagaaaatcatttttacttttttcatttttaaaaacataatcatataacatcttgttcttaaatactcttggtatttgttgtcccataatagatagaacatcattgctatcacaaaaacctttctctcctaaaaaaaccaatgctgcttttgggtgagtacatgatgaattttttaccctttttattatatgtgcaaatgcaatatctgatatgctttcattctttttattgactattctacagtatggtctaacaccatctaaattcatacctacatgactccatatattgttttataaacatcatagtaaagggtgttatattttcatatagtatgcaagcttctttgtatactatgatttaaaaaaacggaacaaaaatgcaattatattctggcattgtttaatgctgtttgtcttttttattcattatttcaatgaaagaatttcaactacaaagttaattgatcctacctacataatatagacacagtacctaaagtaccgtggagatcatccggggaagccccgggtctgataccagtgcaggtagaccgctatcttgttccggtaatgttctgccgttctttacggcatcacctctagcagaaacatcgcgtaactgcgccaagtatgatgaacttctcttaccgctgcgatctggttcaacaatatgcagatggtacacaacctctttaaccagcccatccggattattcagagccgcttccgcaatccggaataaaagtgctgttttaccgtgaaccctctgaaaatcagcgagcagtgttttaaccagccgacgttcagagcgtactgacagacggtggacaatttgaataaacagttcaatcagaccgtagatgctctcccgttggcgacatgctgggagcggtggaacaccgcttcggcaacgagcttccggcgtctccagtgaagtgactgacggataatggttcatgctaccgggctaatgaaacacgccagttcacccggatgttgggactttgttgaaccgaagaacacggcggtgcggagtccggagagtaacagaatagcaaagagcttcgtgaaaacaataaagtgtgactacatcagtatcatgcccaaacctgacgggttaacggcagcaaagaaccttgcagaggcattcgagcattataacgaatggcatccgcatagtgcgctggattatcgctcgccacgggaatatctgcggcagcgggccaatgataacaggtgtctggaaatataggggcaaagccacccacgaacgatccagcatataatcaagaaattagatttcatttaacataagatagattatgtgcaccaagaggcgtttccttaatttctgaatttacaggctcttttgctctctttgctatgctgtggatttggaaagatagagtgctggctgattatttaggtattttcatcagcat

(19) United States
(12) Patent Application Publication
(10) Pub. No.: US 2023/0399650 A1
(43) Pub. Date: Dec. 14, 2023

(54) PLASMID STABILISATION

(71) Applicant: Oxford University Innovation Limited, Oxford (Oxfordshire (GB)

(72) Inventors: Christoph TANG, Oxford (Oxfordshire) (GB); Gareth MCVICKER, Oxford (Oxfordshire) (GB); Sarah HOLLINGSHEAD, Oxford (Oxfordshire) (GB)

(21) Appl. No.: 18/021,564

(22) PCT Filed: Aug. 17, 2021
(86) PCT No.: PCT/GB2021/052124

§ 371 Date: Feb. 16, 2023

(30) Foreign Application Priority Data
Aug. 17, 2020 (GB) 2012839.3

(51) Int. Cl. C12N 15/74; C12N 15/70

(52) U.S. Cl. CPC C12N 15/74; C12N 15/70

(57) ABSTRACT

The invention related to an isolated plasmid encoding a bacterial VapBC toxin-antitoxin system, wherein the VapBC toxin-antitoxin system comprises a modified VapB amino acid sequence arranged to increase the maintenance of the plasmid in a bacterial host, wherein the modification to VapB comprises or consists of a substitution of at least one of Q12, F6, F51 and F60 residues relative to Shigella sonnei wild-type VapB or Shigella flexneri wild-type VapB, or structurally equivalent residues of Shigella sonnei wild-type VapB or Shigella flexneri wild-type VapB in a homologue thereof; and associated methods, uses and host cells.

### CROSS-REFERENCE TO RELATED APPLICATIONS

This application is the National Stage of International Application No. PCT/GB2021/052124, filed Aug. 17, 2021, which claims priority to GB 2012839.3, filed Aug. 17, 2020, which are entirely incorporated herein by reference.

### SEQUENCE LISTING

This application contains a sequence listing filed in electronic form as an ASCII.txt file entitled “Seq listing 1054210WOP_ST25.TXT”, created on Nov. 16, 2021, and having a size of 566 kb. The content of the sequence listing is incorporated herein in its entirety.

### FIELD OF THE DISCLOSURE

The present invention relates to plasmids encoding a VapBC toxin-antitoxin system, wherein the VapBC toxin-antitoxin system is arranged to increase the plasmid stability in a bacterial host, and related nucleic acid, host bacterium, methods and uses thereof.

### BACKGROUND

Shigella spp. are the leading cause of bacillary dysentery worldwide with Shigella sonnei responsible for the majority of cases in developed countries. A Type III secretion system (T3SS) is critical for Shigella spp. virulence, and is encoded by genes located in a 30 kb pathogenicity island (PAI) on a ˜210 kb virulence plasmid, called pINV. This Shigella plasmid is understood to be present as a single copy and is absolutely critical for virulence, as pINV harbours genetic determinants that confer the ability to invade mucosal epithelial cells in the large intestine and cause disease. The other three species of Shigella (Shigella flexneri, Shigella dysenteriae, and Shigella boydii) and enteroinvasive E. coli all contain a related large virulence plasmid. Growth at 37° C. in the laboratory results in frequent loss of virulence in S. sonnei due to loss of the plasmid. Little is known about the mechanisms underlying the high rate of plasmid loss in this important pathogen, even though it hampers studies on the pathogenesis and prevention of S. sonnei infection, and infection with other species of Shigella.

The T3SS acts as a molecular syringe, forming a conduit between the bacterium and the host cell that allows translocation of effectors directly from the pathogen into the host cytoplasm. The T3SS imposes a significant fitness cost upon Shigella during growth in vitro and Shigella can lose expression of the T3SS either through loss of the PAI, loss of pINV in its entirety or integration of pINV into the chromosome. Thus to ensure PAI retention, pINV is naturally maintained in a growing bacterial population through post-segregational killing (PSK) mechanisms, which typically consist of one or more toxin-antitoxin (TA) systems.

TA systems are widespread across bacterial and archaeal genomes and are modules composed of a toxin that is inhibited by the presence of a cognate antitoxin. Protein antitoxins are labile and targeted for degradation by cellular proteases such as Lon and ClpP. For the antitoxin to counteract the effects of the toxin, it must be continually replenished by de novo expression from the TA operon. If the genes encoding the TA system are lost, for example due to failed segregation or following a recombination event, antitoxin levels fall, releasing the toxin, which in the case of Shigella, prevents the growth of plasmid free daughter cells, causing PSK.

The most abundant TA system is the Vap (Virulence associated protein) B and C family, which is broadly distributed across prokaryotes and particularly prevalent in pathogenic bacteria, either as a single copy or in multiple copies. For example, the major human pathogen, Mycobacterium tuberculosis, contains around 50 vapBC loci, while all species of Shigella have a single vapBC locus on the virulence plasmid. The toxin VapC is characterised by a PIN (PilT N-terminus) domain, which is a compact RNA-binding domain that co-ordinates one or more divalent metal ions and is essential for endoribonuclease activity. The antitoxin VapB is usually composed of an N-terminal DNA-binding domain and a C-terminal region that interacts with and inhibits VapC through displacement of divalent metal ions at the active site. The Shigella spp. vapBC allele is located on pINV, where it is essential for plasmid maintenance at 37° C. and functions as a PSK mechanism that acts to retain the pINV in growing bacterial populations. The antitoxin VapB is labile to degradation by Lon protease and less stable than the toxin VapC. In daughter cells that have not inherited pINV, PSK is activated when VapB is degraded by Lon, releasing toxic VapC. Once released from suppression by VapB, the Shigella spp. VapC specifically cleaves the prokaryote initiator tRNA, tRNA formyl methionine (tRNA^{fMet}), thus inhibiting protein translation and halting cell growth.

Understanding the different behaviours of S. sonnei and S. flexneri (which together account for around 90% of cases of shigellosis worldwide), would allow the construction of strains of S. sonnei which retain their virulence in the laboratory. This should facilitate studies of host:pathogen interactions and vaccine design in the future.

Therefore an aim of the present invention is to improve the stability of the S. sonnei and S. flexneri virulence plasmid pINV, and/or other plasmids in bacteria.

### SUMMARY OF THE DISCLOSURE

According to a first aspect of the present invention, there is provided an isolated plasmid encoding a bacterial VapBC toxin-antitoxin system, wherein the VapBC toxin-antitoxin system comprises a modified VapB amino acid sequence arranged to increase the maintenance of the plasmid in the bacterial host, wherein the modification to VapB comprises or consists of a substitution of at least one of Q12, F6, F51 and F60 residues relative to Shigella sonnei wild-type VapB, or structurally equivalent residues of Shigella sonnei wild-type VapB in a homologue thereof.

Advantageously, it has been found that the natural S. sonnei disease isolate CS14, harbours a point mutation in VapB that super-stabilises pINV, preventing its loss from in vitro grown bacterial populations at both environmental and host temperatures. Further modifications have been identified in VapB that also stabilise the plasmid significantly. These findings are relevant for understanding different behaviours of S. sonnei and S. flexneri (which together account for around 90% of cases of shigellosis worldwide), and allow the construction of strains of S. sonnei which retain their virulence in the laboratory. This can facilitate studies of S. sonnei host:pathogen interactions and vaccine design.

VapB

In one embodiment, the modification to VapB comprises or consists of a Q12 mutation relative to Shigella sonnei wild-type VapB, or a structurally equivalent residue of Shigella sonnei wild-type VapB Q12 in a homologue thereof. The homologue may comprise VapB of Shigella flexneri. Therefore, a structurally equivalent residue of Shigella sonnei wild-type VapB Q12 in a homologue thereof may comprise a VapB Q12 mutation, wherein the VapB is of Shigella flexneri. In one embodiment, the modification to VapB comprises or consists of a mutation corresponding to Q12 of SEQ ID NO: 1. Reference to a mutation corresponding to a residue in SEQ ID NO: 1 is understood to refer to SEQ ID NO: 1 as only a reference sequence used to identify the equivalent residue in a VapB homologue.

The Q12 mutation may be a substitution to another amino acid. In one embodiment, the Q12 mutation comprises or consists of a Q12L or Q12A mutation.

Additionally or alternatively, the modification to VapB may comprise or consist of one or more phenylalanine substitutions. The phenylalanine substitutions may comprise or consist of F6 and/or F51, or a structurally equivalent residue of Shigella sonnei wild-type VapB F6 and/or F51 in a homologue thereof. Additionally or alternatively, the phenylalanine substitutions may comprise or consist of F60, or a structurally equivalent residue of Shigella sonnei wild-type VapB F60 in a homologue thereof. In one embodiment, the modification to VapB comprises or consists of mutations corresponding to F6 and/or F51 of SEQ ID NO: 1. The homologue may comprise VapB of Shigella flexneri. Therefore, a structurally equivalent residue of Shigella sonnei wild-type VapB F6 and/or F51 in a homologue thereof may comprise a VapB F6 and/or F51 mutation, wherein the VapB is of Shigella flexneri. A structurally equivalent residue of Shigella sonnei wild-type VapB F60 in a homologue thereof may comprise a VapB F60 mutation, wherein the VapB is of Shigella flexneri.

The phenylalanine substitutions may comprise or consist of a substitution of one or more, or all residues of F6, F51 and F60.

The F6 mutation may be a substitution to another amino acid. In one embodiment the F6 mutation comprises F6A. In another embodiment the F6 mutation comprises F6S or F6G. The F51 mutation may be a substitution to another amino acid. In one embodiment the F51 mutation comprises F51A. In another embodiment the F51 mutation comprises F51S of F51G. The F60 mutation may be a substitution to another amino acid. In one embodiment the F60 mutation comprises F60A. In another embodiment the F60 mutation comprises F60S of F60G.

In one embodiment, the phenylalanine substitutions may comprise or consist of a F6A and/or F51A substitution, or a structurally equivalent substitution in a homologue of Shigella sonnei wild-type VapB. In another embodiment, the phenylalanine substitutions may comprise or consist of a F6S and/or F51S substitution, or a structurally equivalent substitution in a homologue of Shigella sonnei wild-type VapB. In another embodiment, the phenylalanine substitutions may comprise or consist of a F6G and/or F51G substitution, or a structurally equivalent substitution in a homologue of Shigella sonnei wild-type VapB.

In one embodiment, the Shigella sonnei wild-type VapB amino acid sequence comprises or consists of the sequence of SEQ ID NO: 1. In one embodiment, the wild-type VapB from Shigella flexneri may comprise or consist of the sequence of SEQ ID NO: 2.

In one embodiment, the modified VapB amino acid sequence comprises or consists of the sequence of SEQ ID NO: 3. In another embodiment, the modified VapB amino acid sequence comprises or consists of the sequence of SEQ ID NO: 4. In another embodiment, the modified VapB amino acid sequence comprises or consists of the sequence of SEQ ID NO: 5. In another embodiment, the modified VapB amino acid sequence comprises or consists of the sequence of SEQ ID NO: 6. In another embodiment, the modified VapB amino acid sequence comprises or consists of the sequence of SEQ ID NO: 7.

In another embodiment, the modified VapB amino acid sequence comprises or consists of the sequence of SEQ ID NO: 8. In another embodiment, the modified VapB amino acid sequence comprises or consists of the sequence of SEQ ID NO: 9. In another embodiment, the modified VapB amino acid sequence comprises or consists of the sequence of SEQ ID NO: 10. In another embodiment, the modified VapB amino acid sequence comprises or consists of the sequence of SEQ ID NO: 11. In another embodiment, the modified VapB amino acid sequence comprises or consists of the sequence of SEQ ID NO: 12. In another embodiment, the modified VapB amino acid sequence comprises or consists of the sequence of SEQ ID NO: 13.

The homologue of Shigella sonnei wild-type VapB may be a non-Shigella sonnei homologue. In one embodiment, the homologue of Shigella sonnei wild-type VapB may be a non-Shigella spp. homologue. In particular, the homologue may not be a Shigella spp. homologue.

The homologue of Shigella sonnei wild-type VapB may have at least 60% sequence identity with Shigella sonnei wild-type VapB (SEQ ID NO: 1). In another embodiment, the homologue of Shigella sonnei wild-type VapB may have at least 70% sequence identity with Shigella sonnei wild-type VapB (SEQ ID NO: 1). In another embodiment, the homologue of Shigella sonnei wild-type VapB may have at least 75% sequence identity with Shigella sonnei wild-type VapB (SEQ ID NO: 1). In another embodiment, the homologue of Shigella sonnei wild-type VapB may have at least 80% sequence identity with Shigella sonnei wild-type VapB (SEQ ID NO: 1). In another embodiment, the homologue of Shigella sonnei wild-type VapB may have at least 85% sequence identity with Shigella sonnei wild-type VapB (SEQ ID NO: 1). In another embodiment, the homologue of Shigella sonnei wild-type VapB may have at least 90% sequence identity with Shigella sonnei wild-type VapB (SEQ ID NO: 1). In another embodiment, the homologue of Shigella sonnei wild-type VapB may have at least 95% sequence identity with Shigella sonnei wild-type VapB (SEQ ID NO: 1). In another embodiment, the homologue of Shigella sonnei wild-type VapB may have at least 98% sequence identity with Shigella sonnei wild-type VapB (SEQ ID NO: 1). In another embodiment, the homologue of Shigella sonnei wild-type VapB may have at least 99% sequence identity with Shigella sonnei wild-type VapB (SEQ ID NO: 1).

VapB has homologues in other bacteria, including Salmonella spp. and other pathogenic E. coli; enteroaggregative E. coli (pAA), enteropathogenic E. coli, enteroinvasive E. coli (pINV 096). Therefore, such homologues are intended to be encompassed by the present invention, with equivalent amino acid modifications. pDE105 and pAA are resistance plasmids with VapB homologues. The skilled person will recognise that the charged R13 of the pDE105 or pAA VapB homologues can be an equivalent residue for Q12 discussed herein. Therefore, R13 can be a target for the same substitution to another residue, such as A. Similarly, one or more of the phenylalanine substitutions described herein may be provided in the equivalent residues of such homologues. In one embodiment the VapB homologue may comprise or consist of any of SEQ ID NOs: 25 to 32.

The Plasmid

The plasmid may be any plasmid, for example where it would be desirable to enhance stability in a bacterial host.

In one embodiment, the plasmid comprises pINV, for example a modified pINV from Shigella spp. encoding the modified VapBC toxin-antitoxin system according to the invention. Wild type pINV comprises the sequence of SEQ ID NO: 34. Therefore, in one embodiment, the plasmid may comprise the SEQ ID NO: 34, except wherein one or more sequences encoding the wild-type VapBC toxin-antitoxin system are substituted with a modified version thereof according to the invention. In one embodiment, the plasmid comprising the sequence of SEQ ID NO: 34 may be further modified with a sequence insert to encode a product, such as a polypeptide or nucleotide product.

In one embodiment, the plasmid comprises the sequence of SEQ ID NO: 33 (pINV of CS14). In another embodiment, the plasmid comprises the sequence of SEQ ID NO: 33 (pINV of CS14) and further modified to encode one or more phenylalanine substitutions as described herein (e.g. F6, F51 and/or F60). Additionally or alternatively, the plasmid comprising the sequence of SEQ ID NO: 33 may be further modified with a sequence insert to encode a product, such as a polypeptide or nucleotide product.

In another embodiment, the plasmid comprises pSTAB, for example a modified pSTAB encoding the modified VapBC toxin-antitoxin system according to the invention.

The plasmid may be any plasmid of E. coli, Shigella spp., or Salmonella spp., Enterobacter spp., Pseudomonas spp. or Yersinia spp., or other pathogenic bacterial species.

The plasmid may be a recombinant plasmid. In one embodiment, the plasmid is not isolated from wild-type S. sonnei CS14. In one embodiment, the plasmid is not wild-type S. sonnei CS14 pINV.

The plasmid may be an expression plasmid, for example a homologous polypeptide expression plasmid. In one embodiment, the plasmid encodes one or more polypeptides for expression, such as overexpression or delivery of vaccine antigens. The polypeptide for expression may be a homologous polypeptide (i.e. homologous to the bacterial host). The plasmid may comprise a promoter, such as a heterologous promoter, for expression of a polypeptide or nucleotide product.

The plasmid may be an expression plasmid, for example a heterologous polypeptide expression plasmid. In one embodiment, the plasmid encodes one or more polypeptides for expression, such as overexpression or delivery of vaccine antigens. The polypeptide for expression may be a heterologous polypeptide (i.e. heterologous to the bacterial host).

The polypeptide for expression may otherwise be known as an “expression product”. The expression product may be a pharma product, such as a therapeutic molecule. The expression product may comprise a drug peptide, a pro-drug, a toxin, or an antigen for a vaccine. The expression product may comprise a biologic.

The plasmid may be provided in a circular form or a linearised form. The plasmid may be between about 6 kb and about 210 kb in size, or more.

The plasmid may be isolated, or in another embodiment, the plasmid may be in a host bacterial cell. The plasmid may be heterologous to the host bacterial cell.

The Bacterial Host

The bacterial host may comprise any bacterial species that may be used to host a plasmid. The bacterial host may be a Gram-negative bacterium. In one embodiment, the bacterial host is of the phylum: Proteobacteria. In one embodiment, the bacterial host is of the class: Gammaproteobacteria. In one embodiment, the bacterial host is of the order: Enterobacterales. In one embodiment, the bacterial host is of the family: Enterobacteriaceae. The bacterial host may be of the genus: Shigella. The bacterial host may be a modified bacteria (e.g. transformed with the plasmid according to the invention).

In one embodiment the bacterial host comprises a Shigella spp. The Shigella spp. may be Shigella sonnei. In one embodiment, the Shigella spp. is S. sonnei 53G. The Shigella spp. may not be Shigella flexneri. The Shigella spp. may not be S. sonnei CS14, or any other wild-type strain having natural amino acid residue variations that are equivalent to the modifications described herein. The naturally amino acid residues may not comprise one or more of Q12, F6, F51 and F60 (i.e. one or more of such residues may naturally (i.e. wild-type) be an alternative amino acid such as alanine). In another embodiment, the bacterial host is E. coli. The bacterial host may be any bacteria known to have unstable plasmids, such as any of Salmonella, Enterobacter, Pseudomonas, and Yersinia species.

Other Aspects

According to another aspect of the invention, there is provided a bacterial host cell containing a plasmid according to the invention.

According to another aspect of the invention, there is provided a modified bacterial host cell, wherein the modified bacterial host cell is modified to comprise a pINV of a wild-type Shigella spp., such as S. sonnei CS14, wherein one or more of the modifications to the VapBC toxin-antitoxin system described herein are naturally provided.

The modified bacterial host cell may not be a wild-type carrier of the pINV wherein one or more of the modifications to the VapBC toxin-antitoxin system described herein are naturally provided. In one embodiment, the bacterial host cell is not S. sonnei CS14 or wild-type S. sonnei CS14. The bacterial host cell may be selected from Escherichia, Salmonella, Enterobacter, Pseudomonas, and Yersinia species. The bacterial host cell may be Shigella spp. but optionally not S. sonnei CS14. In one embodiment, the bacterial host cell may express lon protease. The bacterial host cell may be a lon+ strain.

According to another aspect of the present invention, there is provided a nucleic acid encoding:
the modified VapBC toxin-antitoxin system described herein; or
the modified VapB described herein.

The nucleic acid may be isolated nucleic acid. The nucleic acid may be recombinant. The nucleic acid may not be derived from S. sonnei CS14.

In one embodiment, the modified VapB comprises or consists of a substitution of at least one of Q12, F6, F51 and F60 residues relative to Shigella sonnei wild-type VapB, or structurally equivalent residues of Shigella sonnei wild-type VapB or in a homologue thereof.

The nucleic acid encoding the modified VapBC toxin-antitoxin system described herein or the modified VapB described herein may or may not be a plasmid. For example the nucleic acid may be a construct encoding the modified VapBC toxin-antitoxin system for insertion into a plasmid. In another embodiment, the nucleic acid may be chromosomal DNA. For example, the VapBC toxin-antitoxin system according to the invention may be chromosomally inserted, for example for use to stabilise/maintain a chromosomal element in a bacterial host cell.

According to another aspect of the present invention, there is provided a modified VapB, the modification comprising or consisting of a substitution of at least one of Q12, F6, F51 and F60 residues relative to Shigella sonnei wild-type VapB, or structurally equivalent residues of Shigella sonnei wild-type VapB in a homologue thereof.

The modified VapB according to the invention may be isolated (i.e. from a cell) or in a host cell.

According to another aspect of the present invention, there is provided a bacterial host cell comprising modified VapB according to the invention.

According to another aspect of the present invention, there is provided the use of
i) a plasmid or nucleic acid according to the invention herein, or
ii) a pINV of a wild-type Shigella spp., such as S. sonnei CS14, wherein one or more of the modifications to the VapBC toxin-antitoxin system described herein are naturally provided;
for stabilising/enhancing the virulence of a bacterial host.

According to another aspect of the present invention, there is provided the use of
i) a plasmid or nucleic acid according to the invention herein, or
ii) a pINV of a wild-type Shigella spp., such as S. sonnei CS14, wherein one or more of the modifications to the VapBC toxin-antitoxin system described herein are naturally provided;
for expression of a protein, e.g. in a host bacterial cell.

According to another aspect of the present invention, there is provided the use of
i) a plasmid or nucleic acid according to the invention herein, or
ii) a pINV of a wild-type Shigella spp., such as S. sonnei CS14, wherein one or more of the modifications to the VapBC toxin-antitoxin system described herein are naturally provided;
for delivery of vaccine antigens encoded on the plasmid of i) or ii).

According to another aspect of the present invention, there is provided the use of the nucleic acid or modified VapB according to the invention for stabilising a plasmid in a bacterial host.

The bacterial host may be engineered to contain the nucleic acid according to the invention, The plasmid to be stabilised/maintained may be recombined with the nucleic acid according to the invention.

The modified bacterial host cell may not be a wild-type carrier of the pINV wherein one or more of the modifications to the VapBC toxin-antitoxin system described herein are naturally provided. In one embodiment, the bacterial host cell is not S. sonnei CS14 or wild-type S. sonnei CS14. The bacterial host cell may be selected from Escherichia, Salmonella, Enterobacter, Pseudomonas, and Yersinia species. The bacterial host cell may be Shigella spp. but optionally not S. sonnei CS14.

The sensitive screening of agents to target plasmids in bacteria requires use of a highly stable plasmid, which has low/negligible levels of plasmid loss.

Therefore, according to another aspect of the present invention, there is provided the use of a modified bacterial host according to the invention herein for screening of antibiotics.

The screening of antibiotics may be for identifying antibiotics that can eliminate or at least reduce plasmids in a bacterial host which confer virulence or resistance to antimicrobials, or both.

According to another aspect of the present invention, there is provided the use of a nucleic acid according to the invention for enhancing the stabilisation of a plasmid in a bacterial host.

According to another aspect of the present invention, there is provided the use of a modified bacterial host according to the invention herein for plasmid-based expression of a product.

According to another aspect of the present invention, there is provided a method of producing a polypeptide or nucleic acid product, the method comprising the cultivation of a bacterial host according to the invention wherein the bacterial host encodes and expresses the polypeptide or nucleic acid product.

The polypeptide or nucleic acid product may be encoded on a plasmid according to the invention, for example, encoding a modified VapB described herein, or encoding a VapBC toxin-antitoxin system described herein. The polypeptide or nucleic acid product may be encoded on a modified pINV of S. sonnei CS14.

According to another aspect of the present invention, there is provided a method of antibiotic screening, the method comprising exposing a modified bacterial host according to the invention herein to one or more agents, and determining if the agents inhibit or kill the bacterial host.

The use or method of antibiotic screening may comprise the determination of the inhibition or killing efficiency of the agent.

The agent may be a small molecule (e.g. less than about 700 Da), a peptide or polypeptide. In one embodiment, the agent may be a known antibiotic. In another embodiment, the agent may be an antibody, antibody mimetic, or antibody fragment.

According to another aspect of the present invention, there is provided a method of modifying a VapBC toxin-antitoxin system encoded in a nucleic acid, the method comprising modifying the nuclei acid to encode a modified VapB described herein.

According to another aspect of the present invention, there is provided a method of stabilising a plasmid in a bacterial host, the method comprising the insertion of the nucleic acid according to the invention into the plasmid; or wherein the plasmid encodes a VapBC toxin-antitoxin system, the method comprising modifying the nuclei acid to encode a modified VapB described herein.

According to another aspect of the present invention, there is provided a nucleic acid encoding any one of the modified VapB polypeptides described herein.

According to another aspect of the present invention, there is provided a bacterial host cell expressing any one of the modified VapB polypeptides described herein.

According to another aspect of the present invention, there is provided the use of the VapBC toxin-antitoxin system described herein to prevent plasmid loss in a bacterial host cell.

The use to prevent plasmid loss may also be known as use as a selection marker.

The use of the VapBC toxin-antitoxin system as a selection marker may be instead of an antibiotic selection marker. The use of the VapBC toxin-antitoxin system as a selection marker may be in combination with an expression of a protein product from a bacterial host cell in the absence of antibiotic supplementation.

According to another aspect of the present invention, there is provided a method of protein product expression from a bacterial host cell, wherein the expression product is encoded on a plasmid that further encodes the VapBC toxin-antitoxin system, for example as described herein.

The method may comprise the culturing of the bacterial host cell to express the protein product. The culturing of the bacterial host cell may be in the absence of an antibiotic to provide selection pressure for the plasmid (i.e. to prevent plasmid loss). The bacterial host cell may be as described herein. The bacterial host cell may be lon+. The method may comprise harvesting the expressed protein product, for example by purification or isolation.

### Definitions

Reference to a “homologue” herein is intended to refer to a polypeptide from another species having shared ancestry and shared function.

Reference to “stabilising” or “stabilisation” of a plasmid herein is intended to refer to the enhanced retention/maintenance of a plasmid by a bacterial host during culture of the bacterial host. The retention may be enhanced relative to the same plasmid not having the modified VapBC toxin-antitoxin system described herein, or relative to a plasmid having a S. sonnei wild-type form of the VapBC toxin-antitoxin system which is not from strain CS14. In one embodiment, the stabilisation is determined by the percentage loss of the plasmid over at least 25 doublings, where at least 99.9% of the population retains the plasmid.

It will be appreciated that the invention extends to any nucleic acid or peptide or variant, derivative or homologue thereof, which comprises substantially the amino acid or nucleic acid sequences of any of the sequences referred to herein, including variants or fragments thereof. The terms “substantially the amino acid nucleotide peptide sequence”, “variant” and “homologue”, can be a sequence that has at least 80% sequence identity with the amino acid/nucleotide/peptide sequences of any one of the sequences referred to herein, for example 80% identity with the nucleic acids or polypeptides described herein. Amino acid/polynucleotide/polypeptide sequences with a sequence identity which is greater than 85%, more preferably greater than 90%, 95%, 98%, and still more preferably greater than 99% sequence identity to any of the sequences referred to are also envisaged. Preferably, the amino acid/polynucleotide/polypeptide sequence has at least 85% identity with any of the sequences referred to, more preferably at least 90%, 92%, 95%, 97%, 98%, and most preferably at least 99% identity with any of the sequences referred to herein. The amino acid/polynucleotide/polypeptide sequence may have 100% identity with any of the sequences referred to herein. Homologues of wild-type VapB of SEQ ID NO: 1 may have at least 80%, 85%, 90%, 95%, 98%, 99% identity with SEQ ID NO: 1. The homologues of wild-type VapB of SEQ ID NO: 1 may have substantially the same antitoxin functional activity as wild-type VapB of SEQ ID NO: 1.

Minor variants (which still have antitoxin function) of the described VapB polypeptides may be provided. Variants of the described nucleotides, including the described plasmids, encoding the VapB polypeptide may be provided. Where reference is made to a variant polypeptide or nucleotide sequence, or a homologue, the skilled person will understand that one or more amino acid residue or nucleotide substitutions, deletions or additions, may be tolerated, optionally two substitutions may be tolerated in the sequence, such that it maintains its function. The skilled person will appreciate that 1, 2, 3, 4, 5 or more amino acid residues or nucleotides may be substituted, added or removed without affecting function. Homologues of wild-type VapB of SEQ ID NO: 1 may include those having 1, 2, 3, 4, 5 or more amino acid residues substituted, added or removed without affecting antitoxin function.

References to sequence identity may be determined by BLAST sequence alignment (www.ncbi.nlm.nih.gov/BLAST/) using standard/default parameters. For example, the sequence may have at least 99% identity and still function according to the invention. In other embodiments, the sequence may have at least 98% identity and still function according to the invention. In another embodiment, the sequence may have at least 95% identity and still function according to the invention. In another embodiment, the sequence may have at least 90%, 85%, or 80% identity and still function according to the invention. In one embodiment, the variation and sequence identity may be according the full length sequence. In other embodiments, the variation may be limited to non-conserved sequences and/or sequences outside of active sites, such as binding domains. Therefore, an active site or binding site of a protein may be 100% identical, whereas the flanking sequences may comprise the stated variations in identity. Such variants may be termed “conserved active site variants”.

Amino acid substitutions apart from the stated substitutions according to the invention may be conservative substitutions (e.g. the substitutions for stabilisation of VapB according to the invention may be non-conservative, and optionally any other variations in amino acid residues may be conservative). For example, a modified residue may comprise substantially similar properties as the wild-type substituted residue. For example, a substituted residue may comprise substantially similar or equal charge or hydrophobicity as the wild-type substituted residue. For example, a substituted residue may comprise substantially similar molecular weight or steric bulk as the wild-type substituted residue. With reference to “variant” nucleic acid sequences, the skilled person will appreciate that 1, 2, 3, 4, 5 or more codons may be substituted, added or removed without affecting function. For example, conservative substitutions may be considered.

The skilled technician will appreciate how to calculate the percentage identity between two amino acid/polynucleotide/polypeptide sequences. In order to calculate the percentage identity between two amino acid/polynucleotide/polypeptide sequences, an alignment of the two sequences must first be prepared, followed by calculation of the sequence identity value. The percentage identity for two sequences may take different values depending on:—(i) the method used to align the sequences, for example, ClustalW, BLAST, FASTA, Smith-Waterman (implemented in different programs), or structural alignment from 3D comparison; and (ii) the parameters used by the alignment method, for example, local versus global alignment, the pair-score matrix used (e.g. BLOSUM62, PAM250, Gonnet etc.), and gap-penalty, e.g. functional form and constants.

Having made the alignment, there are many different ways of calculating percentage identity between the two sequences. For example, one may divide the number of identities by: (i) the length of shortest sequence; (ii) the length of alignment; (iii) the mean length of sequence; (iv) the number of non-gap positions; or (iv) the number of equivalenced positions excluding overhangs. Furthermore, it will be appreciated that percentage identity is also strongly length dependent. Therefore, the shorter a pair of sequences is, the higher the sequence identity one may expect to occur by chance.

Hence, it will be appreciated that the accurate alignment of protein or DNA sequences is a complex process. The popular multiple alignment program ClustalW (Thompson et al., 1994, Nucleic Acids Research, 22, 4673-4680; Thompson et al., 1997, Nucleic Acids Research, 24, 4876-4882) is a preferred way for generating multiple alignments of proteins or DNA in accordance with the invention. Suitable parameters for ClustalW may be as follows: For DNA alignments: Gap Open Penalty=15.0, Gap Extension Penalty=6.66, and Matrix=Identity. For protein alignments: Gap Open Penalty=10.0, Gap Extension Penalty=0.2, and Matrix=Gonnet. For DNA and Protein alignments: ENDGAP=−1, and GAPDIST=4. Those skilled in the art will be aware that it may be necessary to vary these and other parameters for optimal sequence alignment.

Preferably, calculation of percentage identities between two amino acid/polynucleotide/polypeptide sequences may then be calculated from such an alignment as (N/T)*100, where N is the number of positions at which the sequences share an identical residue, and T is the total number of positions compared including gaps but excluding overhangs. Hence, a most preferred method for calculating percentage identity between two sequences comprises (i) preparing a sequence alignment using the ClustalW program using a suitable set of parameters, for example, as set out above; and (ii) inserting the values of N and T into the following formula: −Sequence Identity=(N/T)*100. Alternative methods for identifying similar sequences will be known to those skilled in the art. For example, a substantially similar nucleotide sequence will be encoded by a sequence which hybridizes to any sequences referred to herein or their complements under stringent conditions. By stringent conditions, we mean the nucleotide hybridises to filter-bound DNA or RNA in 3× sodium chloride/sodium citrate (SSC) at approximately 45° C. followed by at least one wash in o.2×SSC/0.1% SDS at approximately 20-65° C. Alternatively, a substantially similar polypeptide may differ by at least 1, but less than 5, 10, 20, 50 or 100 amino acids from the polypeptide sequences described herein.

Due to the degeneracy of the genetic code, it is clear that any nucleic acid sequence described herein could be varied or changed without substantially affecting the sequence of the protein encoded thereby, to provide a variant thereof. Suitable nucleotide variants are those having a sequence altered by the substitution of different codons that encode the same amino acid within the sequence, thus producing a silent change. Other suitable variants are those having homologous nucleotide sequences but comprising all, or portions of, sequence, which are altered by the substitution of different codons that encode an amino acid with a side chain of similar biophysical properties to the amino acid it substitutes, to produce a conservative change. For example, small non-polar, hydrophobic amino acids include glycine, alanine, leucine, isoleucine, valine, proline, and methionine. Large non-polar, hydrophobic amino acids include phenylalanine, tryptophan and tyrosine. The polar neutral amino acids include serine, threonine, cysteine, asparagine and glutamine. The positively charged (basic) amino acids include lysine, arginine and histidine. The negatively charged (acidic) amino acids include aspartic acid and glutamic acid. It will therefore be appreciated which amino acids may be replaced with an amino acid having similar biophysical properties, and the skilled technician will know the nucleotide sequences encoding these amino acids.

Reference to “structurally equivalent” residues in a homologue is intended to refer to residues that will align with the residue of interest in a sequence alignment with S. sonnei wild-type of SEQ ID NO: 1. The aligned residue may be different to the residue of S. sonnei wild-type of SEQ ID NO: 1, but may have the same or similar function. For example it may be a conservative substitution/variation. Additionally or alternatively, the residue may be in a different position in the sequence (i.e. different residue number), but such residue will align in a sequence alignment. Alternatively, the structurally equivalent residue may be the same amino acid, but in a position that is not aligned with S. sonnei wild-type of SEQ ID NO: 1, and no more than 1 or 2 residues away from the residue of interest in S. sonnei wild-type of SEQ ID NO: 1.

The skilled person will understand that optional features of one embodiment or aspect of the invention may be applicable, where appropriate, to other embodiments or aspects ofthe invention.

### BRIEF DESCRIPTION OF THE DRAWINGS

Embodiments of the invention will now be described in more detail, by way of example only, with reference to the accompanying drawings.

FIG. 1. Stabilisation of S. sonnei CS14 pINV is mediated by an amino acid substitution in the antitoxin VapB. (A) Emergence of avirulent CR⁻ colonies at 21° C. during liquid growth of S. sonnei 53G and S. sonnei CS14. The effect of the VapB alleles VapB^{Q12} and VapB^{L12} on PAI loss in S. sonnei 53G and S. sonnei CS14 (B) and on pSTAB stability in S. flexneri M90T and S. sonnei 53G lacking pINV at 37° C. (C) and 21° C. (D). Loss of the PAI or pSTAB was measured by the sacB-neo^{R} assay in strains with indicated VapBC alleles grown at 37° C. and 21° C. (n=9 colonies from at least three independent experiments). Statistical significance determined using one-way and two-way ANOVA: * p<0.0001, ***p=0.0001, ** p=0.0027 and ns p=0.4391.

FIG. 2. VapB^{L12}C has impaired binding to promoter DNA. (A) Schematic of the auto-regulated VapBC bicistronic operon, including the promoter, which contains two VapBC operator sites (O1 and O2). (B) Structure of S. sonnei VapBC hetero-octamer (VapC, blue; VapB, gold; PDB ID: 6SD6). The VapB dimer is indicated, a close-up is shown of the hydrogen bonds made between residue Q12 and the backbone of residue L22. EMSA demonstrating interactions between VapB^{Q12}C or VapB^{L12}C and ds-DNA corresponding to the S. sonnei 53G native vapBC promoter (C) or the constitutive promoter J23101 (D). The effect of auto-regulation on pSTAB stability in S. flexneri M90T and S. sonnei 53G lacking pINV (E). pSTAB loss measured by the sacB-neo^{R} assay in strains expressing indicated VapBC alleles under the control of the native vapBC promoter or the constitutive promoter J23101 (n=9 colonies from at least three independent experiments). Statistical significance calculated using two-way ANOVA: **** p<0.0001.

FIG. 3. VapBC degradation by Lon protease. Stability of pSTAB expressing indicated VapBC alleles in S. flexneri M90T, S. flexneri M90T Δlon (A) and S. flexneri M90T ΔclpP (B) (n=9 colonies from at least three independent experiments). Statistical significance calculated by two-way ANOVA of S. flexneri M90T vs S. flexneri M90T Δlon or S. flexneri M90T vs S. flexneri M90T ΔclpP carrying equivalent vectors: *** p=0.0008 and **** p<0.0001. (C) In vitro proteolysis assays conducted at 37° C. with indicated VapBC proteins and purified Shigella Lon₆ protease in the presence of polyphosphate, vapBC promoter DNA or control DNA (n=3 from three independent experiments). (D) In vivo degradation of VapB; Western blot using anti-FLAG to detect 3×FLAG-tagged VapB^{Q12} and VapB^{L12} in whole cell extracts from S. sonnei 53G treated with rifampicin to arrest protein synthesis.

FIG. 4. Identification of VapB Lon cleavage sites. (A) Location of two Lon cleavage sites in VapB (gold) complexed with VapC (blue). Highlighted in green are the residues flanking the Lon cleavage site (pink dashed line), which is C′ of F51 and F60. (B) The effect of introducing point mutations F6A, F51A and F60A into VapB^{Q12}C and VapB^{L12}C on pSTAB stability in S. flexneri. Each strain was tested on three independent occasions, two-way ANOVA was conducted in GraphPad Prism V8 to show statistical significance of pSTAB loss when expressing different VapBC alleles.

FIG. 5. Sequence alignment of the VapBC locus in S. sonnei 53G and CS14. Alignment of promoter and coding regions of VapBC in S. sonnei strains 53G and CS14. The two VapBC operator sites (O1 and O2; orange), T>C base substitution in VapBC promoter region (grey), VapB coding region (blue), three predicted Lon cleavage sites C′ of phenylalanine (green) and VapC coding region (pink).

FIG. 6. Assessment of VapBC proteins from C. cresecentus and R. felis. (A-D) Plasmid loss assays conducted in E. coli MG1655 at both 21° C. and 37° C. Each dot represents the result for a single colony (n=9 for each strain). Each strain was tested on three independent occasions; horizontal line shows the mean. (A) pSTAB containing S. sonnei VapB^{Q12}C or VapB^{L12}C under the control of either the native VapBC promoter or the constitutive promoter J23101. (B) pSTAB containing C. crescentus wild type VapBC1, VapB^{E15L}C1 or VapB^{E15A}C1 under the control of constitutive promoter J23101. (C) pSTAB containing either R. felis wild type VapBC2, VapB^{Q13L}C1 or VapB^{Q13A}C1 under the control of constitutive promoter J23101.

FIG. 7. Structural comparison of VapBC DNA binding domains and EMSAs conducted with VapBC mutants. (A) Overlay of C. crescentus (pink; PDB ID: 5K8J), R. felis (green; PDB ID: 3ZVK) and S. sonnei 53G (gold; PDB ID: 6SD6) DNA binding domains. EMSAs demonstrating binding of VapB^{Q12}C (B), VapB^{L12}C (C) and VapB^{A12}C (D) to ds-DNA encoding operator site 1 (OS 1).

FIG. 8. Surface plasmon resonance. SPR traces of VapBC binding to operator site 1 for VapB^{Q12}C (A), VapB^{L12}C (B) and VapB^{A12}C (C). (D) Estimation of Kd for VapBC binding to operator site 1.

FIG. 9. Degradation of VapBC by Lon protease. (A) Stability of pSTAB expressing indicated VapBC alleles in S. flexneri M90T and S. flexneri M90T ΔclpP (n=9 colonies from three independent experiments). (B) In vitro proteolysis assays conducted at 37° C. with indicated VapBC proteins and purified Shigella Lon₆ protease with indicated combinations of ATP, polyphosphate, vapBC promoter DNA and control DNA. Each proteolysis experiment was conducted on three separate occasions (n=3). (C) Representative SDS-PAGE gels from Lon proteolysis experiments, VapBC proteins and proteolysis conditions are indicated.

FIG. 10. Intact mass analysis of VapBC proteins. MS spectra of indicated VapBC proteins analysed by intact mass.

FIG. 11: Plasmid map of the pET28a-His-MBP-TEV vector engineered to co-express the VapBC TA system from S. sonnei CS14 and fHbp V1.1 from N. meningitidis.

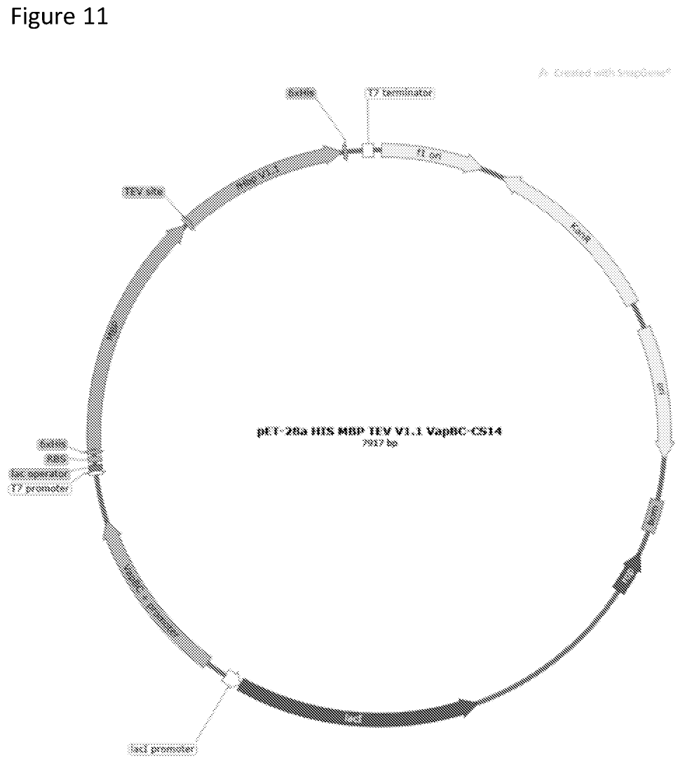

FIG. 12: Initial step of plasmid loss assay. The difference between the number of colonies on a plate with and a plate without the antibiotic, obtained from the same dilution sample, provides a rough estimate of plasmid loss.

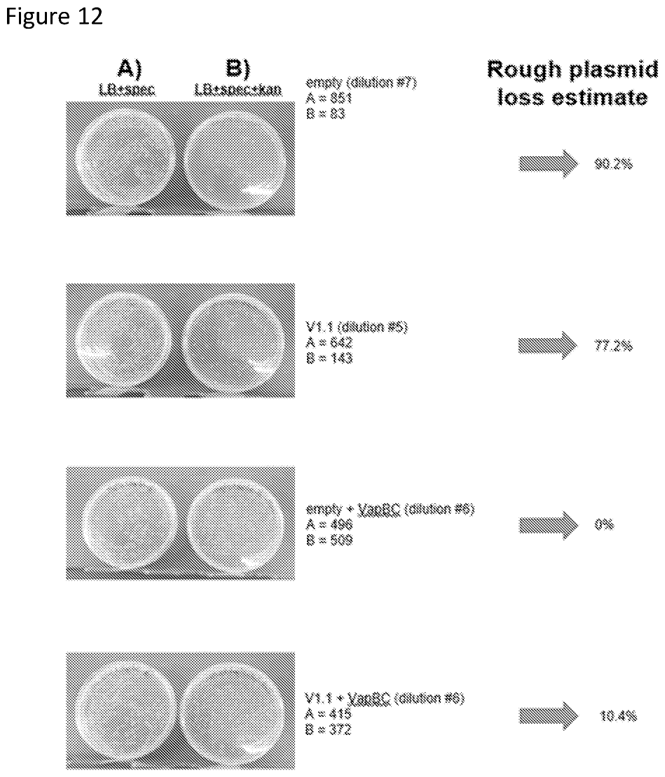

FIG. 13: Final step of plasmid loss assay. The same colonies are streaked onto a plate with and one without antibiotic. The difference between the number of colonies provides a more precise estimate of plasmid loss than the initial step of the experiment.

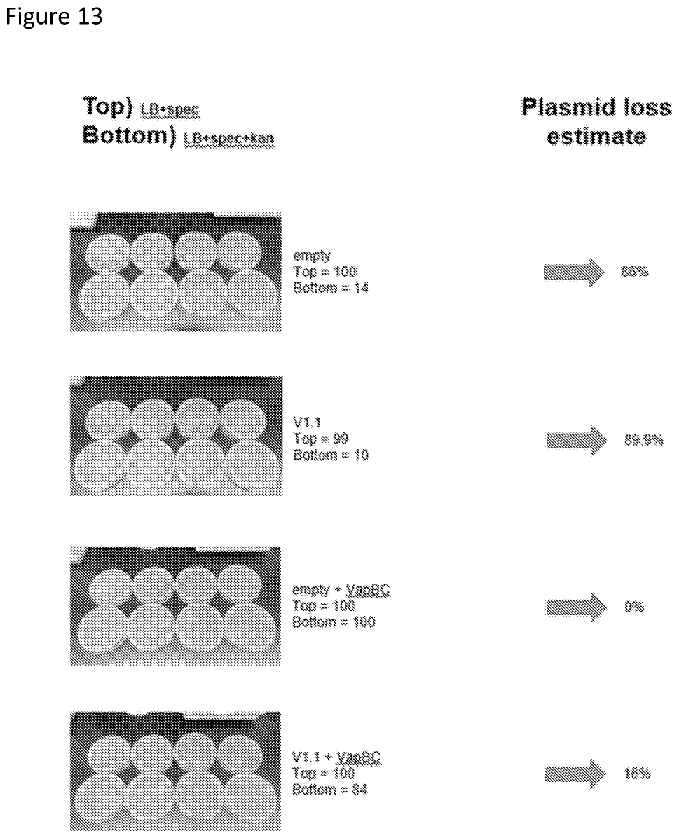

### EXAMPLE 1—MOLECULAR MECHANISMS GOVERNING STABILISATION OF THE S. SONNEI VIRULENCE PLASMID

Summary

Here, we show that the S. sonnei CS14 clinical isolate has a highly stable virulence plasmid that is retained by almost 100% of the growing population. Super-stabilisation is enabled by an amino acid substitution in VapB that prevents loss of pINV from cultures grown in vitro at temperatures found both inside and outside of the host environment.

Materials and Methods

Strains and Growth Media

The bacteria and plasmids used in this study are shown in Supplementary Tables 1 and 2, respectively. Escherichia coli and Shigella spp. were propagated in Luria broth (LB, Invitrogen) or on solid media containing 1.5% (w/v) agar (Oxoid). Antibiotics were used at the following concentrations: carbenicillin, 50 μg ml⁻¹; chloramphenicol, 34 μg ml⁻¹; kanamycin, 50 μg ml⁻¹. Screening for Congo Red positive (CR⁺) and negative (CR⁻) colonies was performed on tryptic soy broth (TSB, Fluka) solid media containing 0.01% (w/v) Congo red (Sigma). Sucrose selection was performed on solid media containing 1% (w/v) tryptone (Fluka), 0.5% (w/v) yeast extract (Fluka), 1.5% (w/v) agar, and 10% sucrose (w/v).

Strain and Plasmid Construction

DNA manipulations were performed in E. coli DH5α. Plasmids were assembled by restriction digestion and ligation, or with NEBuilder HiFi DNA assembly master mix (New England Biolabs). Primers used in this study are shown in Supplementary Table 3. Electroporation was used to transform Shigella with plasmids or linear DNA, while chemically competent E. coli were used for transformation with plasmid DNA. To introduce mutations into Shigella spp., λ Red recombination³⁷⁻³⁸ was performed using PCR products including 1 kb of upstream and downstream flanking sequence. Following λ Red recombination in S. flexneri, P1vir transduction was used to transfer mutations into a clean genetic background⁷; the cat cassette was excised using FLP recombinase encoded by pCP20³⁷. For inserting mutations into the S. sonnei chromosome, pCONJ4s was employed³⁹, using 500 bn of upstream and downstream flanking sequence, with E. coli λpir as the donor. Fragments were amplified from S. sonnei 53G genomic DNA and inserted into pET28a using NdeI/XhoI digestion. Rickettsia felis VapBC1 and Caulobacter crescentus VapBC2 were obtained as codon optimised fragments (GeneArt, Invitrogen). Constructs expressing VapB^{F6A}, VapB^{F51A} or VapB^{F60A} were obtained by overlap PCR.

Protein Purification

VapBC and VapB were purified as previously¹⁶. Following Ni affinity chromatography, proteins were passed over an S200 10/30 Increase column equilibrated with 50 mM Tris pH 8.0, 300 mM NaCl and 1 mM dithiothreitol (DTT).

His-MBP tagged Lon was expressed in E. coli B834. Cultures were grown at 37° C. to an OD₆₀₀˜1, induced with 1 mM IPTG and incubated at 21° C. for 3 hours prior to harvesting. Cell pellets were resuspended in buffer A (50 mM Tris pH8.0, 500 mM NaCl, 1 mM DTT, 10 mM imidazole and 10% glycerol) and lysed by five passes through an Avestin C5 homogeniser at 15,000 p.s.i. Clarified supernatant was passed over a chelating sepharose fast flow column (GE Healthcare) equilibrated with nickel sulphate and buffer A. Resin was washed with 20 column volumes of buffer A, followed by 20 column volumes of buffer A supplemented with 30 mM imidazole. Proteins were eluted with 10 column volumes of buffer A supplemented with 300 mM imidazole. The His-MBP tag was removed by overnight dialysis at 4° C. with TEV protease in buffer A, and passed back through the nickel affinity column to remove the cleaved tag and protease. Flow through containing untagged Lon was concentrated to 1 ml and run over a S200 10/30 Increase gel filtration column equilibrated with 20 mM Tris pH8.0, 200 mM NaCl, 1 mM DTT, 25 mM MgCl₂ and 20% glycerol. Fractions containing Lon were pooled, flash frozen in liquid N₂ and stored at −80° C. until required.

Virulence Plasmid Stability Assays

Plasmid stability assays were performed by red-white screening of colonies on solid media containing Congo red, or with strains containing sacB-neo^{R} in mxiH (which is located in the T3SS PAI), or using pSTAB as described previously 7. For red-white screening, bacteria were grown in liquid culture at 21° C. with subculturing into fresh LB every 24 hours for a total of 96 hours, followed by serial dilution and plating onto solid media containing Congo red at relevant time points. For sacB-neo^{R} assays, bacteria retaining pINV or pSTAB after ˜25 generations of growth on solid media were detected by serially diluting colonies in PBS and plating onto media selecting for kanamycin resistant or sucrose resistant colonies to detect the presence (pSTAB⁺ or PAI⁺) or absence (pSTAB⁻ or PAI⁻) of the sacB-neo^{R} cassette, respectively.

Toxicity Assays

To assess the toxicity of R. felis VapC1 and C. crescentus VapC2, genes encoding the proteins were ligated into pBAD33 and expressed under the control of an arabinose inducible promoter. Cognate VapB proteins were cloned into pET28a under the control of an IPTG inducible promoter. E. coli B834 containing both plasmids was grown overnight at 37° C. in LB supplemented with 1% glucose (v/v), diluted to an optical density (OD₆₀₀) of 0.01, then grown to an OD₆₀₀ of 0.1 at either 21° C. or 37° C. Bacteria were pelleted, washed with LB to remove glucose, resuspended in LB containing arabinose and IPTG to induce VapC and VapB expression, respectively, then incubated at 21° C. or 37° C. Time points were taken at T=0, 15, 30, 60, 120, 180 and 240 minutes (at 21° C., time points at 300 and 360 minutes were included because of slower growth). The number of colony forming units (CFU) was determined by plating serial dilutions of bacteria onto solid medium containing glucose and IPTG, and incubation at 37° C. overnight.

Sensitivity to Lon Proteolysis

In vitro hexameric Lon (Lon₆; 166 nM) was combined with 2.5 μM VapBC octamer, 8 mM adenosine triphosphate (ATP, Sigma), 10 mM MgCl₂ and 50 mM Tris pH 8.0 in a total volume of 100 μl. Assays were incubated at 37° C. and samples immediately combined with SDS-PAGE loading dye then incubated at 100° C. for 10 minutes⁴⁰. To assess proteolysis in the presence of DNA and polyphosphate, assays were supplemented with 1.5 μM double stranded DNA (dS-DNA) or 1 mM polyphosphate 700 (Kerafast). Samples were resolved by SDS-PAGE, and the gels were stained with Coomassie blue.

To monitor degradation of VapB in vivo, overnight cultures of S. flexneri M90T wild-type and Δlon were used to inoculate 25 ml Luria broth to an OD₆₀₀ 0.01. Cultures were grown to an OD₆₀₀ of ˜2.0 at 37° C. shaking at 180 r.p.m. Translation was arrested by addition of rifampicin (50 μg ml⁻¹), and samples were collected at 0, 5, 10, 20, 30 and 60 minutes post translation inhibition, pellets harvested and flash frozen in liquid N₂. Samples were resolved on 16% SDS-PAGE and transferred to PVDF membranes by dry blotting. To detect FLAG tagged VapB and RecA, blots were probed with α-FLAG (1 in 1000) and α-RecA (1 in 5000). Following incubation with a HRP-conjugated secondary antibody, blots were visualised using an LAS4000 with ECL reagent.

Statistical and Computational Methods

Data analysis was performed in GraphPad Prism. Data were log-transformed and assessed for normal distribution. Data were then analysed using one-way or two-way ANOVA with appropriate multiple comparisons tests as indicated in the Figure legends. Statistical significance was assumed if p<0.05. Quantification of Coomassie stained SDS-PAGE gels was performed using the BioRad gel doc and Image Lab software.

DNA Binding Assays

ds-DNA was generated by annealing equimolar amounts of complementary primers over a gradually reducing temperature from 99° C. to 20° C. Assays were performed as described¹⁶ with VapBC (final concentration range, 0 to 605 nM) and ds-DNA (final concentration, 88 nM) diluted in 20 mM Tris pH7.5, 100 mM KCl, 2 mM MgCl₂, 1 mM DTT, 10% glycerol. Reactions were incubated at 37° C. for 20 minutes, resolved on 7% TBE acrylamide gels supplemented with 10% glycerol, stained with SybrSafe and imaged on a GelDoc system.

Surface plasmon resonance was performed on a Biacore® 3000 instrument at 25° C. Biotinylated ds-DNA (SH167 annealed with SH181) was coupled to streptavidin on the sensor surface (GE Healthcare) at equivalent levels, resulting in a 100 to 150-RU increase. VapBC was then flowed over the chip surface in 50 mM Tris pH8.0, 150 mM NaCl, and 0.005% (v/v) Tween-20 at a flow rate of 40 μl min⁻¹ followed by injection of 10 μL 0.5M NaCl to remove any residual nonspecific interactions. A control trace was collected using an uncoupled channel. Kinetic fits were performed on data from two separate chips, and two batches of purified VapBC (one starting at 3.2 μM and the other at 2.7 μM). A K_{d} estimate for VapBC binding to DNA was taken as the average value after kinetic fits.

Results

A Point Mutation in vapB Enhances Plasmid Stability Conferred by VapBC

Virulent Shigella spp. express the T3SS during growth at 37° C., but can become avirulent through loss of the virulence plasmid pINV or by failing to express the T3SS^{4-5, 41} Virulent and avirulent Shigella can be differentiated by their colony morphology on media containing the dye Congo-red (CR); virulent bacteria bind CR giving rise to red (CR⁺) colonies, whilst avirulent Shigella cannot bind CR and form white (CR⁻) colonies⁴². When plated onto CR-containing agar at 37° C., a significant proportion of S. sonnei colonies are CR⁻ with around 10% of S. sonnei 53G colonies CR⁻ and avirulent⁷. Therefore we investigated whether S. sonnei isolates vary in their spontaneous rate of avirulence, and examined the phenotype of a number of clinical isolates on media containing CR. Intriguingly, we noticed that the clinical isolate S. sonnei CS14 formed no CR⁻ colonies when plated onto CR-agar⁴³.

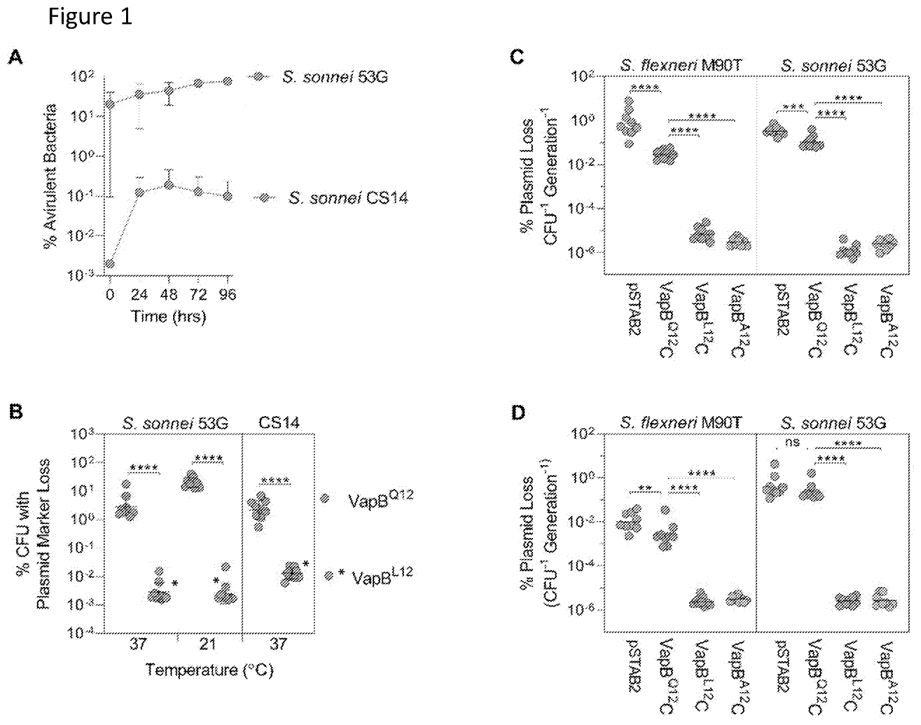

In S. sonnei, pINV is typically retained to a higher degree in bacteria growing at 37° C. than at 21° C.; incubation at 21° C. results in rapid loss of pINV from a growing population⁷. To investigate plasmid maintenance in more detail, we examined S. sonnei CS14 after growth at 21° C. for 96 hours and monitored the appearance of CR⁻ colonies. After 96 hours of continuous culture, almost all S. sonnei CS14 colonies expressed the T3SS with ≤0.1% of colonies appearing CR⁻. In contrast. ≥25% of S. sonnei 53G colonies were CR⁻ (FIG. 1A). Therefore, we sought to define the mechanism that leads to pINV maintenance by S. sonnei CS14.

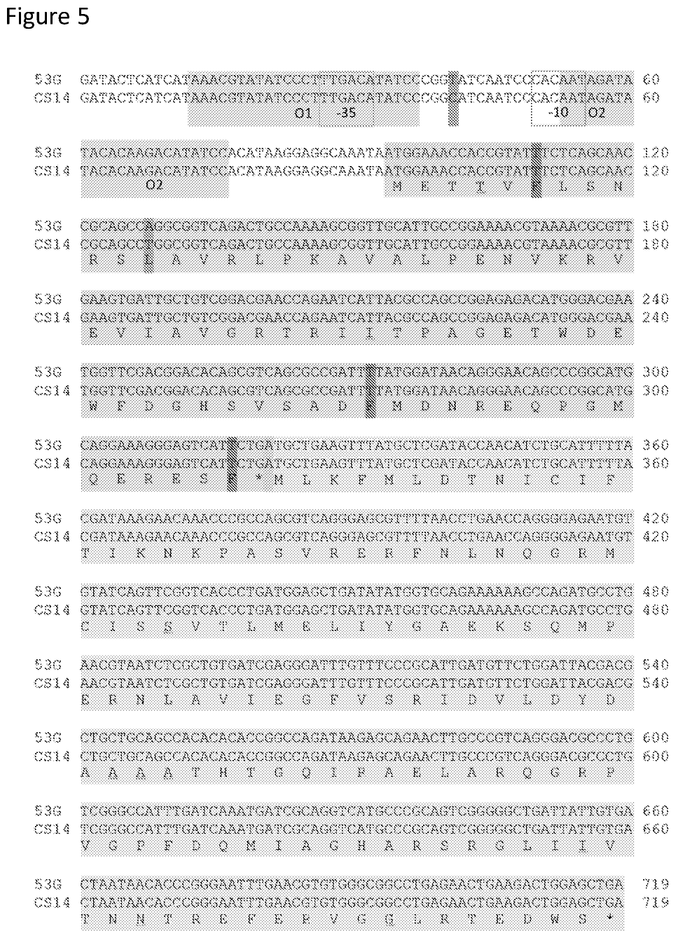

As TA systems are essential for maintenance of pINV within a growing population, we speculated that a change in TA function or repertoire could stabilise S. sonnei CS14 pINV. S. sonnei pINV contains two TA systems, VapBC and RelBE, with VapBC essential for pINV maintenance under laboratory conditions at 37° C. 7. Comparison of the vapBC loci in S. sonnei 53G and CS14 revealed two single nucleotide polymorphisms (SNPs, FIG. 5); there is a T/C substitution located at nucleotide −24 between the −10 and −35 promoter sites, and a non-synonymous A/T SNP, which leads to a Q12L substitution in VapB (generating VapB^{L12}). To whether either/both of these SNPs is responsible for pINV stability, we switched vapB alleles in S. sonnei CS14 (introducing VapB^{L12}) and S. sonnei 53G (introducing VapB^{Q12}), and left the promoter unchanged; to allow for selection, a chloramphenicol resistance cassette (cat) was inserted downstream of vapC. As controls, we generated strains carrying their native vapB allele with cat at the identical position. Stability of the pINV PAI was monitored at 37° C. by placing a sacB-neo^{R} marker in mixH, which is located in the T3SS PAI 7. Remarkably, we found that inserting VapB^{L12} into S. sonnei 53G reduced PAI loss during growth at 37° C. and 21° C. by approximately 1,000- and 10,000-fold, respectively (both p<0.0001; FIG. 1B). Similarly, introduction of VapB^{Q12} into S. sonnei CS14 significantly increased PAI loss at 37° C. by over 100-fold (p<0.0001; FIG. 1B). At 21° C., sucrose-based selection was unsuccessful in S. sonnei CS14. Instead we measured the emergence of CR⁻ colonies to assess plasmid loss at this temperature. Loss of pINV in S. sonnei CS14 carrying the VapB^{Q12} allele reached approximately 10% over 25 generations, approximately four-fold higher than at 37° C., while there was no detectable plasmid loss in the strain carrying the VapB^{L12} allele. Together these data demonstrate that the VapB alleles in S. sonnei CS14 and 53G determine the rate of plasmid loss, with the non-synonymous SNP in VapB from S. sonnei CS14 is necessary and sufficient to stabilise pINV at both 37° C. and 21° C.

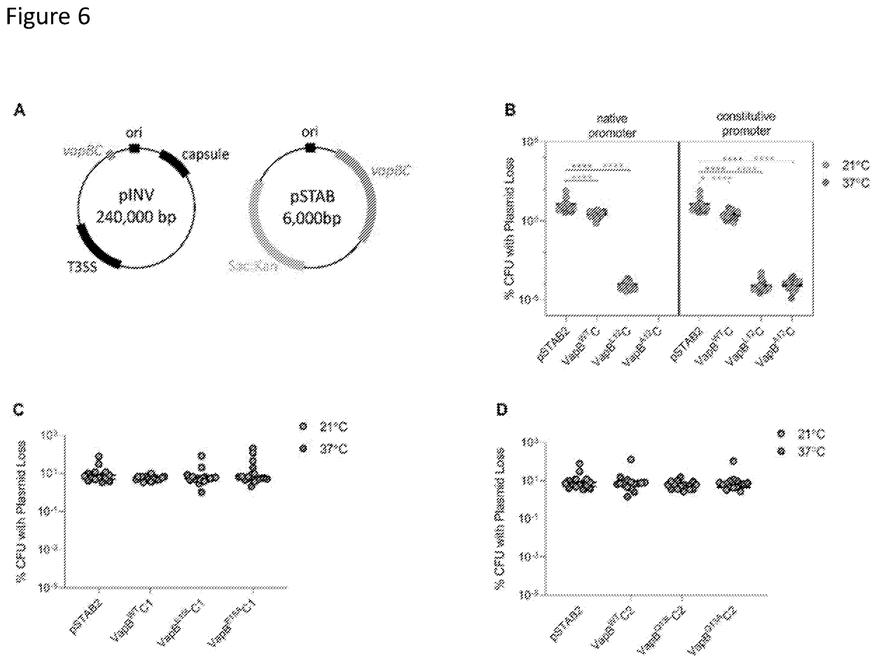

To further investigate the effect of VapBC alleles on plasmid maintenance, we employed the model vector pSTAB (FIG. 6)¹¹, which harbours the on from pINV and a sacB-neo^{R} marker; this allows for testing of individual plasmid maintenance systems in isolation¹¹. We inserted the VapBC alleles from S. sonnei 53G or S. sonnei CS14 (vapB^{Q12}C and vapB^{L12}C, respectively) into pSTAB; we also constructed vapB^{A12}C, which lacks the leucine R group at this residue. Plasmid loss was subsequently monitored in S. flexneri M90T and S. sonnei 53G lacking pINV at 37° C. and 21° C. (FIGS. 1C and 1D). We found that both vapB^{L12}C and vapB^{A12}C significantly stabilised pSTAB in both S. sonnei 53G and S. flexneri M90T by over 10,000-fold at 37° C. and 21° C. compared with vapB^{Q12}C (all p<0.0001; FIG. 1C). Loss of pSTAB containing vapB^{L12}C or vapB^{A12}C is at the limit of detection, so we were unable to identify differences in plasmid stabilisation conferred by these two alleles.

Consistent with previous work, we found that effect of VapBC in stabilising pSTAB varies with the temperature¹¹. In S. flexneri M90T, VapB^{Q12}C significantly stabilised pSTAB at both 21° C. and 37° C. (p<0.0001 and p=0.0027, respectively), with 10-fold greater stabilisation at 37° C. than 21° C. (FIGS. 1C and 1D). In S. sonnei 53G, VapB^{Q12}C significantly stabilised pSTAB at 37° C., but not at 21° C. (p=0.0001 and p=0.4391, respectively; FIGS. 1C and 1D).

To investigate the ability of VapB^{L12}C to stabilise plasmids in other species, pSTAB expressing containing vapB^{Q12}C or vapB^{L12}C was introduced into E. coli MG1655. Plasmid loss assays demonstrate that VapB^{Q12}C and VapB^{L12}C significantly stabilised pSTAB at 37° C. (p=0.051 and p<0.0001) and 21° C. (both p<0.0001; FIG. 6). Taken together our data demonstrate that VapB^{L12}C dramatically stabilises pSTAB independent of the genetic background and temperature (FIGS. 1C and 1D and FIG. 6).

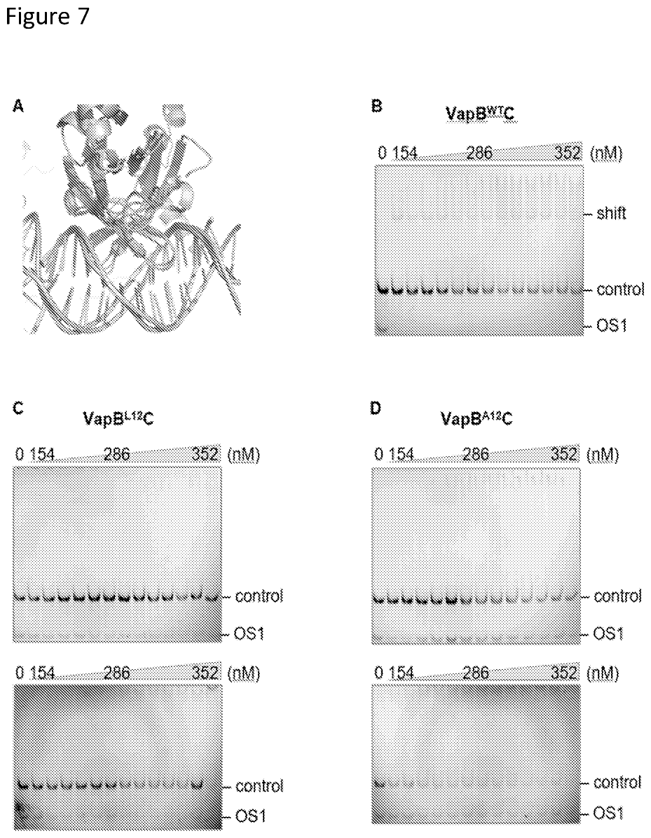

The VapBC TA system is widespread across prokaryotes. Two well characterised VapBC systems are VapBC2 from Rickettsia felis and VapBC1 from Caulobacter crescentus, structures of both these systems bound to their corresponding promoter DNA are available in the Protein Data Bank (PDB; FIG. 7)^{16, 44}. To ascertain if plasmid maintenance via PSK is a common function shared by other VapBC TA systems, we investigated if VapBC1 or VapBC2 could stabilise pSTAB in E. coli. To avoid promoter incompatibility, both VapBC1 and VapBC2 were placed under the control of the constitutive promoter J23101 and point mutations corresponding to L12 or A12 were inserted into both VapB1 and VapB2. Although VapC2 and VapC1 are toxic when expressed in E. coli, we observed that neither VapBC2 nor VapBC1 could induce maintenance of pSTAB in E. coli (FIG. 6). As such, it would appear that the Shigella spp. VapBC TA system is unique its ability to promote plasmid maintenance via PSK.

VapB^{L12}C Exhibits Reduced Binding to the vapBC Promoter, but this does not Affect PSK

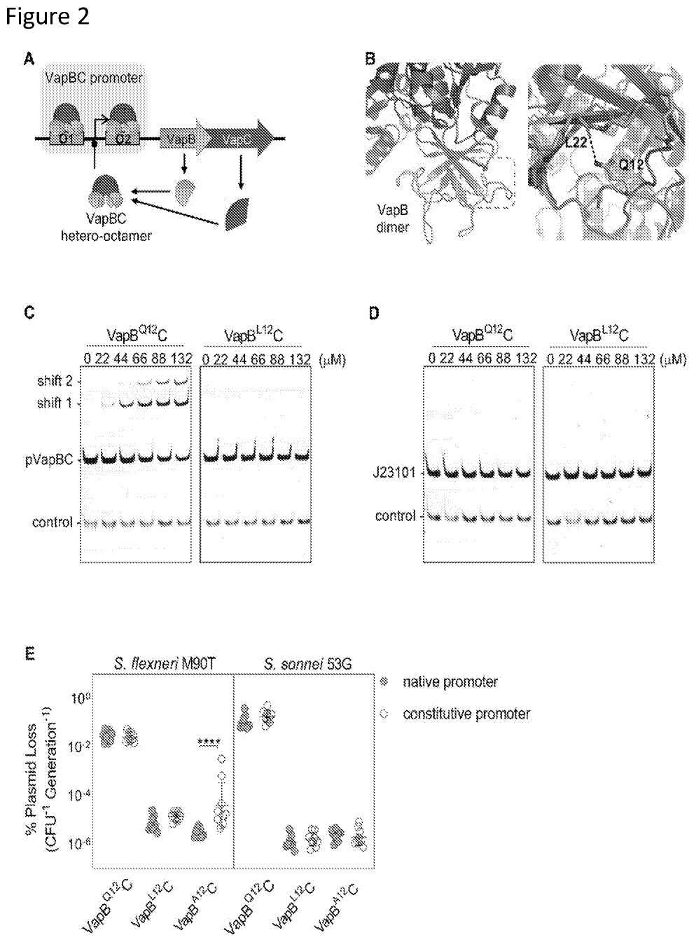

Similar to many Type II TA systems, VapBC is auto-regulated at the transcriptional level through direct binding of the antitoxin to two operator sequences located within the vapBC promoter (FIG. 2A)⁴⁵. Within the VapBC hetero-octamer, two distinct DNA binding domains are formed by VapB dimers making a swapped 0-hairpin fold (FIG. 2B and FIG. 7)¹⁶. The Q12 residue in one VapB forms a hydrogen bond with the backbone of residue L22 in another VapB, stabilising the two interweaved VapB molecules (FIG. 2B). Given the role of Q12 in assembly of the DNA binding domain, we next examined whether VapB^{L12}C and VapB^{A12}C are impaired in DNA binding by electrophoretic mobility shift assays (EMSAs) and surface plasmon resonance (SPR).

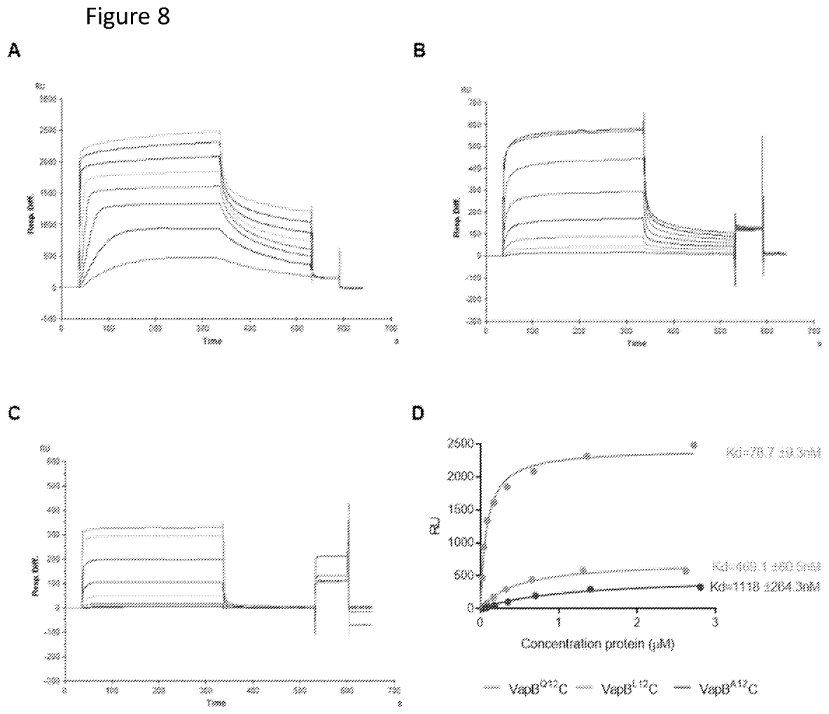

EMSAs were performed with ds-DNA incorporating the two VapBC operator sites (FIG. 2C and FIG. 7), while SPR was performed with two ds-DNA fragments including Operator Site 1 (OS1) or Operator Site 2 (OS2; FIG. 8). EMSAs demonstrate both VapB^{L12}C and VapB^{A12}C are greatly impaired in binding to promoter DNA in comparison to VapB^{Q12}C (FIG. 7). We sought to determine the affinity of the different VapBC proteins for operator DNA by SPR; as the traces for VapB^{Q12}C and VapB^{L12}C did not reach equilibrium (likely due to non-specific binding), we were unable to determine the Kₐ(FIG. 8). However, these data do suggest that the affinity of VapB^{L12}C and VapB^{A12}C for OS1 is reduced by around four-fold and ten-fold, respectively, in agreement with EMSAs (FIG. 8).

These observations led to the following model; reduced binding of VapB^{L12}C or VapB^{A12}C to the promoter would relieve auto-repression, resulting in higher VapBC levels within bacteria, and the increased VapBC concentrations could increase PSK and thence enhance plasmid maintenance. To test this hypothesis we inserted the three vapBC alleles (vapB^{Q12}C, vapB^{L12}C and vap^{A12}C) into pSTAB but under the control of the constitutive promoter J23101⁴⁶; this promoter is not bound by VapBC (FIG. 2C) so would not be subject to autoregulation. Subsequent plasmid loss assays conducted in S. flexneri M90T and S. sonnei 53G lacking pINV showed that VapB^{L12} and VapB^{A12} enhance plasmid maintenance compared to VapB^{Q12}C irrespective of the promoter (FIG. 2E), demonstrating that the loss of auto-regulation is not the mechanism by which VapB^{L12} and VapB^{A12} increase PSK.

VapB Degradation by Lon Protease

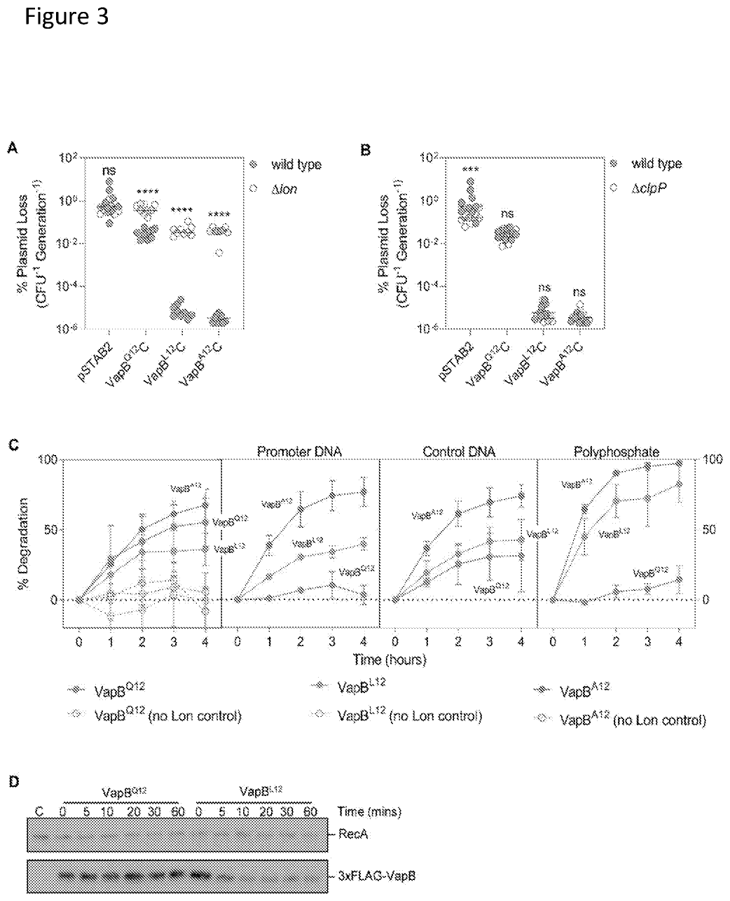

The contribution of TA systems to PSK depends on the preferential degradation of antitoxin by cellular proteases following plasmid loss. This will liberate free toxin to arrest cell growth/cause death, and culminate in PSK. As loss of auto-regulation appears not to mediate plasmid stabilisation, we next examined if VapB^{Q12} and VapB^{L12} exhibit altered degradation. Shigella VapB is degraded by Lon, an ATP-dependent, AAA⁺ protease^{11, 23}. To investigate the role of Lon in the maintenance of pSTAB expressing VapB^{Q12}C, VapB^{L12}C or VapB^{A12}C, we compared the level of plasmid loss between strains S. flexneri M90T and S. flexneri M90T lacking Lon (S. flexneri Δlon, FIG. 3A). The resulting plasmid loss assays demonstrate that pSTAB expressing any version of VapBC is lost more frequently in the absence of Lon, indicating that Lon is essential for PSK (for all plasmid, p<0.0001 comparing wild-type S. flexneri with S. flexneri Δlon; FIG. 3A). However, although loss of Lon is sufficient to abolish VapB^{Q12}C mediated maintenance of pSTAB, VapB^{L12}C or VapB^{A12}C still contribute to pSTAB maintenance in comparison to empty pSTAB (both p<0.0001). Therefore, to investigate if another cellular protease contributes to the maintenance of pSTAB expressing VapB^{L12}C or VapB^{A12}C, we performed plasmid loss assays in S. flexneri M90T ΔclpP; results demonstrate that ClpXP nor ClpAP impact VapBC maintenance of pSTAB (all p≥0.6900; FIG. 3B).

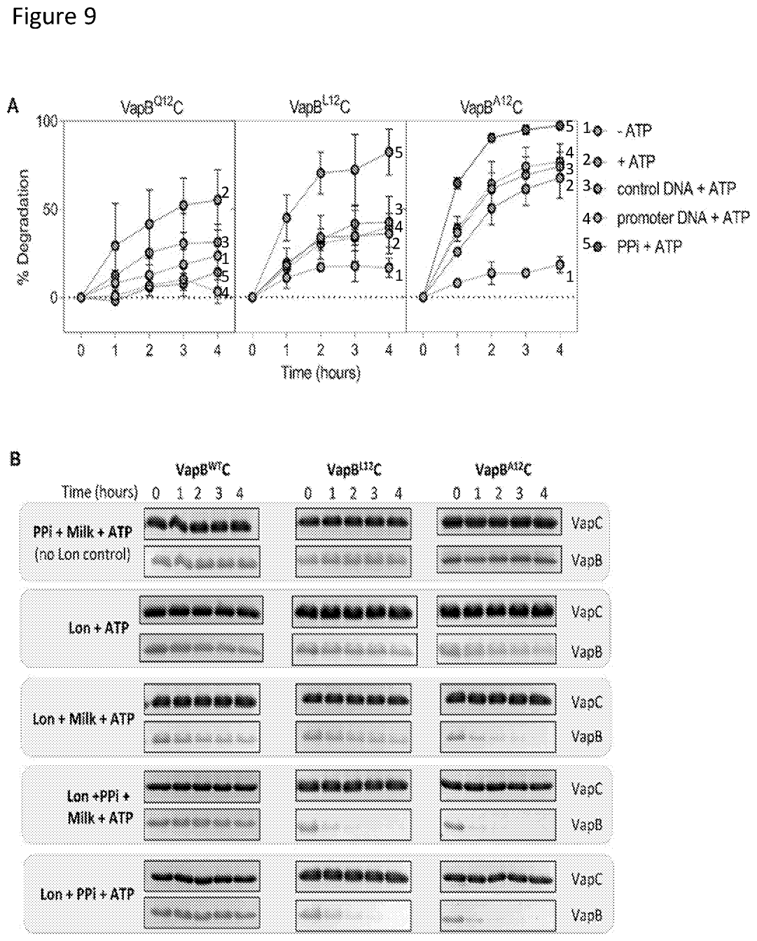

The plasmid loss assays suggest that VapB^{L12}C and VapB^{A12}C could be more sensitive to proteolytic cleavage than VapB^{Q12}C. Therefore, we performed in vitro degradation assays using purified hexameric Lon (Lon₆) and VapBC hetero-octamers, monitoring levels of VapB and VapC hourly for a total of four hours by SDS-PAGE; assays were performed in the presence or absence of ATP with results demonstrating that VapB degradation by Lon protease is ATP dependent (FIG. 9). However there was no significant difference in the degradation of VapB^{Q12} and VapB^{L12} by Lon protease over the course of assays (p>0.12; FIG. 3C), although after two hours we observed that VapB^{A12} was degraded significantly more than VapB^{L12} (p<0.03; FIG. 3C).

As VapBC hetero-octamers bind DNA via two VapB DNA binding domains (FIG. 2A-B), we hypothesised that the presence of DNA could sterically inhibit Lon, impairing the degradation of VapB. To test this, we performed proteolysis assays in the presence of DNA containing the two VapBC operator sites (operator DNA; FIG. 2B) or control DNA (taken from a non-promoter intergenic region). In the presence of operator DNA, we observed virtually no degradation of VapB^{Q12} over the course of assays (p≥0.67; FIG. 3C), whilst in the presence of control DNA, significant degradation of VapB^{Q12} was observed within two hours (p<0.037; FIG. 3C). As expected, neither operator DNA nor control DNA affect Lon degradation of VapB^{L12} or VapB^{A12} (which fail to bind operator sites, p≥0.12; FIG. 3C). In vivo Lon protease can bind DNA which modulates Lon activity. In our assays, we noticed that less VapB^{Q12} is degraded in the presence of control DNA compared to without DNA (31% vs 55% degradation after 4 hours, p=0.036; FIG. 9). This suggests that in vivo DNA could non-specifically affect Lon degradation of VapB^{Q12} that is complexed in hetero-octamers not bound to DNA.

Polyphosphate is known to influence the activity of Lon, changing its substrates specificity²⁵. To determine whether polyphosphate influences VapB degradation, proteolysis assays were performed in the presence of long-chain polyphosphate (average chain length, 700 units). We found that polyphosphate mediated striking differences in Lon degradation of the different VapB proteins (FIG. 3C). Polyphosphate significantly reduced Lon degradation of VapB^{Q12} (p<0.0338, after one hour of degradation±polyphosphate) but significantly enhanced Lon degradation of both VapB^{L12} and VapB^{A12} (p<0.0058 and p<0.0001 after two hours of degradation polyphosphate, respectively; FIG. 9).

To further investigate the turnover of VapB in vivo, we monitored the stability of FLAG-VapB^{Q12} and FLAG-VapB^{A12} in transcription arrested S. sonnei 53G. The resulting Western blots demonstrate that over a time course of 60 minutes FLAG-VapB^{L12} is considerably less stable in vivo than FLAG-VapB^{Q12} (FIG. 3D). The differential degradation of VapB^{Q12} and VapB^{L12} in vivo suggests that cellular factors, such as DNA or polyphosphate, influence Lon mediated degradation of VapB in vivo.

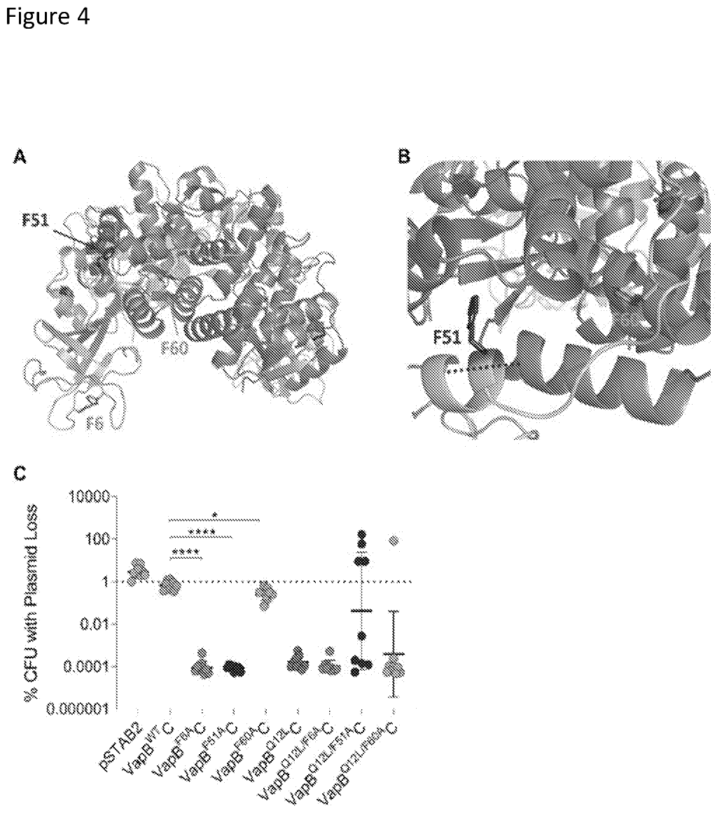

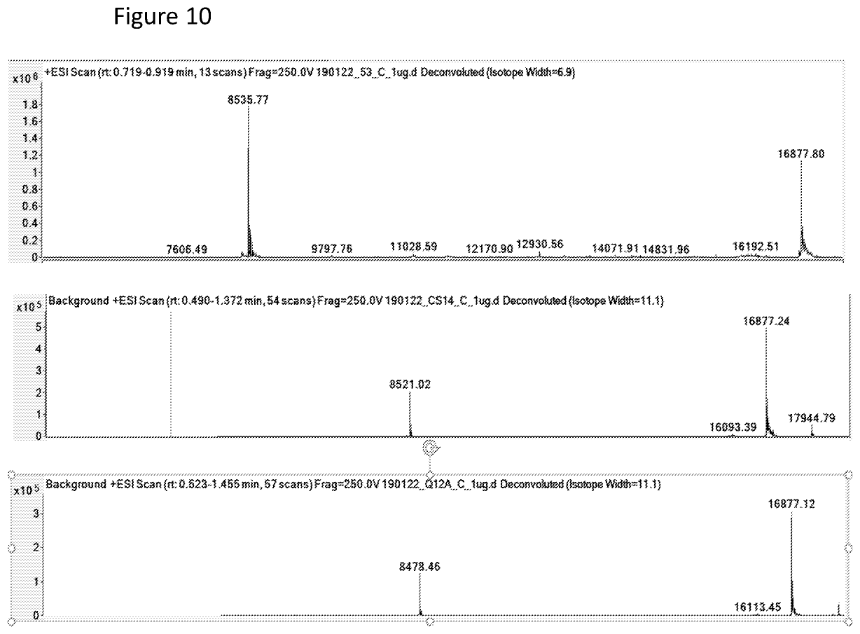

VapB is cleaved by Lon protease¹¹, but the sites of cleavage are unknown. We used mass spectrometry to analyse tryptic peptides of VapB cleaved by Lon. Our analysis shows that all the VapB variants (VapB^{Q12}, VapB^{L12} and VapB^{A12}) contain two Lon cleavage sites situated at the C′ side of phenylalanine residues F51 and F60 (FIGS. 4A and B, and FIG. 10). These data indicate that the substitution of Q12 for an alanine or a leucine does not alter or generate new Lon cleavage sites in VapB. To confirm F51 and F60 as sites of Lon cleavage, we generated a set of pSTAB vectors expressing VapB^{Q12}C or VapB^{L12}C, in which F51 and F60 were mutated to alanine. In addition we also substituted F6 with an alanine, as we obtained MS data that suggests the presence of a third Lon cleavage site C′ of this residue. The resulting plasmid loss assays were unexpected, as VapB^{F6A}C and VapB^{F51}A, significantly stabilised pSTAB in S. flexneri, while the introduction of F60A into VapB^{Q12}C and VapB^{L12}C did not alter plasmid stability (FIG. 4C); we were unable to generate a double F51A/F60A mutant. Examination of the structure of VapBC indicates that F6 and F51 form key hydrophobic interactions that could stabilise the DNA binding domain and interactions between VapB and VapC, respectively. Thus, alanine substitution of these amino acids are likely to destabilise the VapBC hetero-octamer, potentially resulting in the release of toxic VapC regardless of the occurrence of Lon cleavage, which would increase in plasmid stability.

### DISCUSSION

Many enteric pathogens, including the four species of Shigella harbour large, low-copy number plasmids that are essential for virulence. The Shigella virulence plasmid pINV is non-conjugative, so the only way the plasmid is maintained in a bacterial population is by its vertical transmission from mother to daughter cells during cell division. To ensure that important plasmid-encoded genetic traits are retained, plasmids encode PSK mechanisms that act to eliminate bacteria that fail to inherit a plasmid during division. However, the mechanisms mediating PSK, which are typically TA systems, are often incompletely effective, as is the case for S. sonnei, which loses pINV at a high rate during growth in vitro. Here, we identified a disease isolate of S. sonnei, which remarkably almost never loses pINV, even during prolonged and rapid growth. The spontaneous emergence of an invasive enteric pathogen from which the virulence plasmid, is almost never lost has important implications regarding both the intensity and duration of disease, and also, the spread and persistence of disease within the human population.

Based on clinical severity, antibiotic resistance and disease burden, shigellosis caused by Shigella spp. is a prime target for vaccine development⁴⁷⁻⁴⁸. Indeed, Shigella has proven highly effective in acquiring antibiotic resistance to antimicrobials used clinically to reduce both the severity of disease and excretion of the pathogen²⁹⁻³³. The development of a Shigella vaccine would contribute significantly towards reducing the burden of disease, especially in nations where delivery of effective antimicrobials is challenging. To provide effective coverage, any vaccine developed should elicit protection against the most epidemiologically important Shigella serotypes, one of which is S. sonnei. This species is the most important serotype in industrialised counties and accounts for ˜23% of disease isolates in less wealthy countries 47. Unfortunately, vaccine development is limited by the high proportion of S. sonnei cells that spontaneously loose pINV during in vitro growth. For example, vaccines containing live-attenuated strains and lipopolysaccharide 0-antigens both require homogenous expression of plasmid encoded antigens from in vitro grown S. sonnei 35. The inclusion of the pINV stabilising mutations VapB^{L12} or VapB^{A12} in Shigella strains used for vaccine production could contribute significantly towards vaccine development.

The protease Lon belongs to the AAA⁺ superfamily and is required for maintaining protein homeostasis across all domains of life⁴⁹. Lon proteases are classified as Lon A or Lon B, with Shigella Lon an example of the former⁵⁰. Lon A proteases are composed of three functional domains: N-terminal, ATPase and protease. Where, the N-terminal domain is thought to be involved in substrate binding⁵, the ATPase actively unfolds substrates and translocates them into the protease domain⁵², which preferentially cleaves sequences rich in aromatic residues²³⁻²⁴. In this work we demonstrate that Lon can cleave VapB when it is in a complex with VapC, similar to the HipBA TA system⁵³. However, in the CcdAB TA system, sequestration of the antitoxin (CcdA) by the toxin (CcdB) prevents Lon degradation of CcdA²⁴. Therefore, the activation of TA systems is not necessarily dependent on the dissociation of antitoxin and toxin, prior to antitoxin degradation. Nevertheless, in common with the antitoxins CcdA, HipB and VapB is that their sequestration in higher order complexes does block Lon degradation. In the case of CcdA (as mentioned earlier) this is through its association with CcdB, whilst with HipB and VapBC this occurs upon formation of a higher order TA:DNA complex^{24, 53}. Furthermore, our findings demonstrate that polyphosphate modulates Lon substrate specificity for VapB. Our bioinformatic analysis detected no structural homology between Lon (PDB ID: 6U5Z) and either of the known polyphosphate binding domains, the C-terminus of PPX1⁵⁴ or the CHAD domain⁵⁵ (PDB ID: 2FLO and 3E0S, respectively), suggesting that Lon may bind polyphosphate via an as yet uncharacterised domain or mechanism. Importantly our data highlights that polyphosphate can modulate both the substrate specificity and proteolytic activity of Lon on highly similar variants of the same protein, which could have implications for how SNPs can influence the Lon regulon under conditions in which polyphosphate is produced.

TABLES
Supplementary Table 1: Bacterial strains used in this study

| Strain | Relevant genotype/description | Reference |
|---|---|---|
| S. sonnei 53G | Wild-type S. sonnei 53G | 56 |
| S. flexneri M90T | Wild type S. flexneri serotype 5a | 57 |
| BS176 | S. flexneri lacking pINV | 57 |
| S. sonnei pINV | S. sonnei lacking pINV | 7 |
| S. sonnei CS14 | Wild type S. sonnei CS14 | 43 |
| E. coli MG1655 | Wild type E. coli K-12 | 58 |
| E. coli C41 | ompT hsdSB (rB− mB−) gal dcm (DE3) | 59 |
| E. coli DH5α | fhuA2 Δ(argF- | 60 |
| | lacZ) U169 phoA glnV44 Φ80 Δ(lacZ)M15 gyrA96 | |
| | recA1 relA1 endA1 thi-1 hsdR17 | |
| E. coli B834 | F⁻ ompT hsdS_{B}(r_{B}⁻ m_{B}⁻) gal dcm met (DE3) | 61 |
| S. flexneri M90T | Δlon | This work |
| Δlon | | |
| S. flexneri M90T | ΔclpP | This work |
| ΔclpP | | |
| S. flexneri M90T | Δppk::cat | This work |
| Δppk | | |
| S. sonnei 53G Δppk | Δppk | This work |
| S. sonnei CS14 | Δppk | This work |
| Δppk | | |
| GMCT307 & | S. sonnei 53G mxiH::sacB-neo^{R} VapB^{Q12}-cat | This work |
| GMCT311 | (two independently clones) | |
| GMCT309 & | S. sonnei 53G mxiH::sacB-neo^{R} VapB^{L12}-cat | This work |
| GMCT312 | (two independently-created clones) | |
| GMCT332 & | S. sonnei CS14 mxiH::sacB-neo^{R} VapB^{L12}-cat | This work |
| GMCT333 | (two independently-created clones) | |
| GMCT334 & | S. sonnei CS14 mxiH::sacB-neo^{R} VapB^{Q12}-cat | This work |
| GMCT335 | (two independently-created clones) | |

SUPPLEMENTARY TABLE 2
Plasmids used in this study

| Plasmid | Description | Reference |
|---|---|---|
| pET28a-VapCB | Expression of His-VapC and VapB^{Q12/L12/A12} | This work |
| pET28a- | | |
| VapCB^{L12} | | |
| pET28a- | | |
| VapCB^{A12} | | |
| pET28a-MBP- | For expression of His-MBP-Lon | This work |
| Lon | | |
| pSTAB2 | 6.3 kb plasmid containing the pINV ori and | ¹¹ |
| | sacB-neo | |
| pSTAB2-VapBC | pSTAB with S. sonnei VapB^{Q12/L12/A12}C | This work |
| pSTAB2- | | |
| VapB^{A12}C | | |
| pSTAB2- | | |
| VapB^{A12}C | | |
| pSTAB2- | pSTAB with S. sonnei VapB^{F6A/F51A/F60A}C | This work |
| VapB^{F6A}C | | |
| pSTAB2- | | |
| VapB^{F51A}C | | |
| pSTAB2- | | |
| VapB^{F60A}C | | |
| pUC19-Lon:cam^{R} | For disruption of Lon protease with cam cassette | ¹¹ |
| pCP20 | FLP recombinase for λ Red cassette excision | ³⁷ |
| pKD3 | Source of camR cassette for λ Red recombination | ³⁸ |
| pKD46 | Helper plasmid for λ Red recombination | ³⁸ |
| pUC19 | High-copy cloning vector | Ref: PMID |
| | | 2985470 |
| pGM195 | pUC19 with VapB^{Q12}-cat and 53G flanking sequence | This study |
| | for creation of GMCT307 & GMCT311 | |
| pGM197 | pUC19 with VapB^{L12}-cat and 53G flanking sequence | This study |
| | for creation of GMCT309 & GMCT312 | |
| pGM203 | pUC19 with VapB^{L12}-cat and CS14 flanking sequence | This study |
| | for creation of GMCT332 & GMCT333 | |
| pGM205 | pUC19 with VapB^{Q12}-cat and CS14 flanking sequence | This study |
| | for creation of GMCT334 & GMCT335 | |

Primers used in this study

| | | Vector |
|---|---|---|
| Primer | Sequence (5′ - 3′) | constructed |
| SH10 | CTGGTGCCGCGCGGCAGCCATATGCTGAAGTTTATGCTC | pET28a-VapCB |
| | G | |
| SH11 | CCATTATTTGCCTCCTTATCAGCTCCAGTCTTCAGTTC | |
| SH12 | CTGGAGCTGATAAGGAGGCAAATAATGGAAACCACCGT | |
| | ATTTC | |
| SH13 | AGTGGTGGTGGTGGTGGTGCTCGAGTCAGAATGACTCCC | |
| | TTTC | |
| | CAGCAACCGCAGCCTGGCGGTCAGACTGCCA | pET28a-VapCB^{L12} |
| | GTCTGACCGCCAGGCTGCGGTTGCTGAGAAATAC | |
| SH134 | GTCTGACCGCCGCGCTGCGGTTGCTGAGAAATAC | pET28a-VapCB^{A12} |
| SH135 | CAGCAACCGCAGCGCGGCGGTCAGACTGCCA | |
| SH24 | CTTGCGGCCGCATGAATCCTGAGCGTTCTGA | pET28a-MBP-Lon |
| SH25 | GTGCTCGAGCTATTTTGCAGTCACAACCTG | |
| GM415 | CCATATGGCTAGCATGAGCGGATCCGTAATTATCCTTGT | pSTAB2-VapBC |
| | CGGTATAATA | |
| GM394 | GGTGCTCGAGTGCGGCCGCAAGCTTGAACAGGTCAGCT | |
| | CCAG | |
| GP65 | AACCGCAGCGCGGCGGTCAGACTGCCAAAA | pSTAB2-VapB^{A12}C |
| GP66 | TCTGACCGCCGCGCTGCGGTTGCTGAGAAATA | |
| GP67 | AACCGCAGCCAGGCGGTCAGACTGCCAAAA | pSTAB2-VapB^{L12}C |
| GP68 | TCTGACCGCCTGGCTGCGGTTGCTGAGAAATA | |
| SH101 | GTTGCTGAGAGCTACGGTGGTTTCCATTATT | pSTAB2-VapB^{F6A}C |
| SH102 | AACCACCGTAGCTCTCAGCAACCGCAGC | |
| SH103 | GTGTCCGTCGGCCCATTCGTCCCATGTCTCTC | pSTAB2-VapB^{F51A}C |
| SH104 | GGACGAATGGGCCGACGGACACAGCGTCAGCG | |
| SH105 | GTTATCCATAGCATCGGCGCTGACGCTGTGTC | pSTAB2-VapB^{F60A}C |
| SH106 | CAGCGCCGATGCTATGGATAACAGGGAACAGC | |
| SH136 | CCATATGGCTAGCATGAGCGGATCCTTTACAGCTAGCTC | pSTAB2-J23101- |
| | AGTCCTAGGTATTATGCTAGCACCCGTTTTTTTGGGCTA | VapBC |
| | GC | |
| SH137 | CCATATGGCTAGCATGAGCGGATCCTTTACAGCTAGCTC | |
| | AGTCCTAGGTATTATGCTAGCACCCGTTTTTTTGGGCTA | |
| | GC | |
| SH85 | GTAAAACGACGGCCAGTGTTTAAACGAAGTGTTCAACT | pCONJ4-Δppk |
| | GGCTGC | |
| SH86 | ATCGTGTATTGGCATAGGGTTATCATTACCACTTTTACTC | |
| | CGTTTTATTCAC | |
| SH87 | TGAATAAAACGGAGTAAAAGTGGTAATGATAACCCTAT | |
| | GCCAATACACGAT | |
| SH88 | GCAGGAAACAGCTATGACGTTTAAACGTACATTTGCATA | |
| | GTGGTATCCA | |
| SH107 | GTAAAACGACGGCCAGTGTTTGAATCGGAAATCGATCT | pCONJ4-lon^{K362Q} |
| | GCT | |
| SH108 | TGGACTGACCAAGAGAGGTTTGACCTACCCCCGGCGGC | |
| | CCTAC | |
| SH109 | GTAGGGCCGCCGGGGGTAGGTCAAACCTCTCTTGGTC | |
| SH110 | GCAGGAAACAGCTATGACGTTTATAATGGCGCTATCGTC | |
| | G | |
| SH111 | AGTAAAACGACGGCCAGTGTTTAAATTCAGTCCCGGGTG | pCONJ4-ppk^{E245K} |
| | C | |
| SH112 | AGACTGGAAGACATCAACTTCATCAGGCTGGCTTCCAT | |
| SH113 | GAGATGGAAGCCAGCCTGATGAAGTTGATGTCTTCCAGT | |
| SH114 | GCAGGAAACAGCTATGACGTTTATGTTGGCTTCTTCGTC | |
| | GA | |
| SH1 | TGGGCTAGCGAATTCGAGCTCAAGAAGGAGATATACCA | pBAD33-C. |
| | TGGCCTATGTTCTGGATACCAATGTTG | crescentus VapC1 |
| SH2 | TGCATGCCTGCAGGTCGACTCTAGATTACCATGCCAGCA | |
| | GGCTC | |
| SH3 | TGGGCTAGCGAATTCGAGCTCAAGAAGGAGATATACCA | pBAD33-R. felis |
| | TGATCTATATGCTGGATACCAAC | VapC2 |
| SH4 | TGCATGCCTGCAGGTCGACTCTAGATCATTTATCCCAGT | |
| | TTTCC | |
| SH5 | ACTTTAAGAAGGAGATATACATGGCACGTGCAACCGGT | pET28a-C. crescentus |
| SH6 | AGTGGTGGTGGTGGTGGTGCTCGAGTTACAGACCAGGA | VapB1 |
| | CGTTCCG | |
| SH7 | ACTTTAAGAAGGAGATATACATGAACAAAGCCAAAATC | pET28a-R. felis |
| | TTTATG | VapB2 |
| SH8 | AGTGGTGGTGGTGGTGGTGCTCGAGTTATTCAAAGTATT | |
| | TACGTTTCTG | |
| SH138 | CCATATGGCTAGCATGAGCGGATCCGAAGGCAGAAAAC | pSTAB-R. felis |
| | TAAGAAAATTAG | VapBC2 |
| SH139 | GGTGCTCGAGTGCGGCCGCAAGCTTATTTATCCCAATTT | |
| | TCTAAAATTAAATTAG | |
| SH140 | TCAAAGTCTGGCAGTAAGATTACCTAAGG | pSTAB-R. felis |
| SH141 | ATCTTACTGCCAGACTTTGACCATTCATAAATATTTTTG | VapB^{Q13L}C2 |
| SH142 | TCAAAGTGCAGCAGTAAGATTACCTAAGG | pSTAB-R. felis |
| SH143 | ATCTTACTGCTGCACTTTGACCATTCATAAATATTTTTG | VapB^{Q13A}C2 |
| SH144 | TGGGCTAGCGAATTCGAGCTCGGCGCGGGGGCGATCTC | pSTAB-C. crescentus |
| | G | VapBC1 |
| SH145 | GGTGCTCGAGTGCGGCCGCAAGCTTCACCAGGCCAGCA | |
| | GTGACAGG | |
| SH146 | CAATAGCCTGGCCGTACGTCTGCCGCGC | pSTAB-C. crescentus |
| SH147 | GACGTACGGCCAGGCTATTGCCGCTTCTGAACGTTTTGC | VapB^{E15L}C1 |
| SH148 | CAATAGCGCAGCCGTACGTCTGCCGCGC | pSTAB-C. crescentus |
| SH149 | GACGTACGGCTGCGCTATTGCCGCTTCTGAACGTTTTGC | VapB^{E15A}C1 |
| SH150 | TCATCATAAACGTATATCCCTTTGACATATCCCGGTATC | EMSA: VapBC |
| | AATCCCACAATAGATATACACAAGACATATCCACATAA | promoter region |
| | GGA | |
| SH159 | TATTGGCTCCTTCTGATGCCATTCTATTTCAGGAAGGAG | EMSA: control DNA |
| | TGTCCGTTAAACTCAGGCTACCTCAGACT | |
| SH152 | ATCATAAACGTATATCCCTTTGACATATCCCG | EMSA: binding site 1 |
| SH156 | CCCACAATAGATATACACAAGACATATCCACAT | EMSA: binding site 2 |
| GP007 | ACGGCCAGTGAATTCGAGCTCGTGAAGCGGGTCCGGGT | Upstream sequence for |
| | G | integration of vapB |
| GP010 | CCATGGCTAATTCCCATTCAGCTCCAGTCTTCAGTTCTCA | mutations |
| | G | |
| GM174 | TGTGTAGGCTGGAGCTGCTT | cat cassette from |
| GM175 | ATGGGAATTAGCCATGGTCC | pKD3 |
| GM292 | CAGCTCCAGCCTACACACCTGTTCATCAGAAATCATCTC | Downstream sequence |
| | C | for integration of |
| GM293 | CTATGACCATGATTACGCCAAGCTTGGAAGCCCGTAAGT | vapB mutations |
| | TCGG | |
| GM294 | GGCAGTCTGACCGCCAGGCTGCGGTTGCTGAGAAATAC | Mutagenic primers for |
| GM295 | CTCAGCAACCGCAGCCTGGCGGTCAGACTGCCAAAAGC | vapB^{L12} construction |
| | G | |
| SH176 | ATGTGGATATGTCTTGTGTATATCTATTGTGGG | 5′ Biotinylated |
| SH177 | AGTCTGAGGTAGCCTGAGTTTAACGGACACTCCTTCCTG | primers |
| | AAATAGAATGGCATCAGAAGGAGCCAATA | for SPR |
| SH180 | ATCATAAACGTATATCCCTTTGACATATCCCG | |
| SH181 | CCCACAATAGATATACACAAGACATATCCACAT | |

### REFERENCES

1. Frost, L. S.; Leplae, R.; Summers, A. O.; Toussaint, A., Mobile genetic elements: the agents of open source evolution. Nature Reviews Microbiology 2005, 3 (9), 722-732.
2. Johnson, T. J.; Nolan, L. K., Pathogenomics of the Virulence Plasmids of <em>Escherichia coli</em>. Microbiology and Molecular Biology Reviews 2009, 73 (4), 750.
3. Lan, R.; Lumb, B.; Ryan, D.; Reeves, P. R., Molecular evolution of large virulence plasmid in Shigella clones and enteroinvasive Escherichia coli. Infect Immun 2001, 69 (10), 6303-9.
4. Schroeder, G. N.; Hilbi, H., Molecular pathogenesis of Shigella spp.: controlling host cell signaling, invasion, and death by type III secretion. Clin Microbiol Rev 2008, 21 (1), 134-56.
5. Buchrieser, C.; Glaser, P.; Rusniok, C.; Nedjari, H.; D'Hauteville, H.; Kunst, F.; Sansonetti, P.; Parsot, C., The virulence plasmid pWR100 and the repertoire of proteins secreted by the type III secretion apparatus of Shigella flexneri. Mol Microbiol 2000, 38 (4), 760-71.
6. Pilla, G.; Tang, C. M., Going around in circles: virulence plasmids in enteric pathogens. Nature Reviews Microbiology 2018, 16 (8), 484-495.
7. McVicker, G.; Tang, C. M., Deletion of toxin-antitoxin systems in the evolution of Shigella sonnei as a host-adapted pathogen. Nat Microbiol 2016, 2, 16204.
8. Tam, J. E.; Kline, B. C., The F plasmid ccd autorepressor is a complex of CcdA and CcdB proteins. Mol Gen Genet 1989, 219 (1-2), 26-32.
9. Gerdes, K.; Christensen, S. K.; Lobner-Olesen, A., Prokaryotic toxin-antitoxin stress response loci. Nat Rev Microbiol 2005, 3 (5), 371-82.
10. Harms, A.; Brodersen, D. E.; Mitarai, N.; Gerdes, K., Toxins, Targets, and Triggers: An Overview of Toxin-Antitoxin Biology. Mol Cell 2018, 70 (5), 768-784.
11. McVicker, G.; Hollingshead, S.; Pilla, G.; Tang, C. M., Maintenance of the virulence plasmid in Shigella flexneri is influenced by Lon and two functional partitioning systems. Mol Microbiol 2019, 111 (5), 1355-1366.
12. Dubiel, A.; Wegrzyn, K.; Kupinski, A. P.; Konieczny, I., ClpAP protease is a universal factor that activates the parDE toxin-antitoxin system from a broad host range RK2 plasmid. Sci Rep 2018, 8 (1), 15287.
13. Sala, A.; Bordes, P.; Genevaux, P., Multiple toxin-antitoxin systems in Mycobacterium tuberculosis. Toxins (Basel) 2014, 6 (3), 1002-20.
14. Sayeed, S.; Reaves, L.; Radnedge, L.; Austin, S., The stability region of the large virulence plasmid of Shigella flexneri encodes an efficient postsegregational killing system. J Bacteriol 2000, 182 (9), 2416-21.
15. Senissar, M.; Manav, M. C.; Brodersen, D. E., Structural conservation of the PIN domain active site across all domains of life. Protein Sci 2017, 26 (8), 1474-1492.
16. Dienemann, C.; Boggild, A.; Winther, K. S.; Gerdes, K.; Brodersen, D. E., Crystal structure of the VapBC toxin-antitoxin complex from Shigella flexneri reveals a hetero-octameric DNA-binding assembly. J Mol Biol 2011, 414 (5), 713-22.
17. Sayeed, S.; Brendler, T.; Davis, M.; Reaves, L.; Austin, S., Surprising dependence on postsegregational killing of host cells for maintenance of the large virulence plasmid of Shigella flexneri. J Bacteriol 2005, 187 (8), 2768-73.
18. Winther, K. S.; Gerdes, K., Enteric virulence associated protein VapC inhibits translation by cleavage of initiator tRNA. Proc Natl Acad Sci USA 2011, 108 (18), 7403-7.
19. Gerdes, K.; Maisonneuve, E., Bacterial persistence and toxin-antitoxin loci. Annu Rev Microbiol 2012, 66, 103-23.
20. Bonnefoy, E.; Almeida, A.; Rouviere-Yaniv, J., Lon-dependent regulation of the DNA binding protein HU in Escherichia coli. Proc Natl Acad Sci USA 1989, 86 (20), 7691-5.
21. Torres-Cabassa, A. S.; Gottesman, S., Capsule synthesis in Escherichia coli K-12 is regulated by proteolysis. J Bacteriol 1987, 169 (3), 981-9.
22. Ishii, Y.; Amano, F., Regulation of SulA cleavage by Lon protease by the C-terminal amino acid of SulA, histidine. Biochem J 2001, 358 (Pt 2), 473-80.
23. Nishii, W.; Suzuki, T.; Nakada, M.; Kim, Y. T.; Muramatsu, T.; Takahashi, K., Cleavage mechanism of ATP-dependent Lon protease toward ribosomal S2 protein. FEBS Lett 2005, 579 (30), 6846-50.
24. Van Melderen, L.; Thi, M. H.; Lecchi, P.; Gottesman, S.; Couturier, M.; Maurizi, M. R., ATP-dependent degradation of CcdA by Lon protease. Effects of secondary structure and heterologous subunit interactions. J Biol Chem 1996, 271 (44), 27730-8.
25. Kuroda, A.; Nomura, K.; Ohtomo, R.; Kato, J.; Ikeda, T.; Takiguchi, N.; Ohtake, H.; Kornberg, A., Role of inorganic polyphosphate in promoting ribosomal protein degradation by the Lon protease in E. coli. Science 2001, 293 (5530), 705-8.
26. Kuroda, A.; Murphy, H.; Cashel, M.; Kornberg, A., Guanosine tetra- and pentaphosphate promote accumulation of inorganic polyphosphate in Escherichia coli. J Biol Chem 1997, 272 (34), 21240-3.
27. Kotloff, K. L.; Winickoff, J. P.; Ivanoff, B.; Clemens, J. D.; Swerdlow, D. L.; Sansonetti, P. J.; Adak, G. K.; Levine, M. M., Global burden of Shigella infections: implications for vaccine development and implementation of control strategies. Bull World Health Organ 1999, 77 (8), 651-66.
28. Lima, I. F.; Havt, A.; Lima, A. A., Update on molecular epidemiology of Shigella infection. Curr Opin Gastroenterol 2015, 31 (1), 30-7.
29. Wong, M. R.; Reddy, V.; Hanson, H.; Johnson, K. M.; Tsoi, B.; Cokes, C.; Gallagher, L.; Lee, L.; Plentsova, A.; Dang, T.; Krueger, A.; Joyce, K.; Balter, S., Antimicrobial Resistance Trends of Shigella Serotypes in New York City, 2006-2009. Microbial Drug Resistance 2010, 16 (2), 155-161.
30. Mamishi, S.; Arab Yazdi, Z.; Mahmoudi, S.; Moradzadeh, M.; Taghi Haghi Ashtiani, M.; Pourakbari, B., Antimicrobial-resistance pattern of Shigella species in children: a six-year study in an Iranian referral Hospital. Ann Ig 2019, 31 (4), 356-364.
31. Sethuvel, D. P. M.; Perumalla, S.; Anandan, S.; Michael, J. S.; Ragupathi, N. K. D.; Gajendran, R.; Walia, K.; Veeraraghavan, B., Antimicrobial resistance, virulence & plasmid profiles among clinical isolates of Shigella serogroups. Indian J Med Res 2019, 149 (2), 247-256.
32. Kang, H.; Wang, L.; Li, Y.; Lu, Y.; Fan, W.; Bi, R.; Qian, H.; Gu, B., Dissemination of Multidrug-Resistant Shigella flexneri and Shigella sonnei with Class 1, Class 2, and Atypical Class 1 Integrons in China. Microb Drug Resist 2019.
33. Bhattacharya, D.; Sugunan, A. P.; Bhattacharjee, H.; Thamizhmani, R.; Sayi, D. S.; Thanasekaran, K.; Manimunda, S. P.; Ghosh, A. R.; Bharadwaj, A. P.; Singhania, M.; Roy, S., Antimicrobial resistance in Shigella—rapid increase & widening of spectrum in Andaman Islands, India. The Indian journal of medical research 2012, 135 (3), 365-370.
34. Bhattacharya, S. K.; Sur, D., An evaluation of current shigellosis treatment. Expert Opin Pharmacother 2003, 4 (8), 1315-20.
35. Gerke, C.; Colucci, A. M.; Giannelli, C.; Sanzone, S.; Vitali, C. G.; Sollai, L.; Rossi, O.; Martin, L. B.; Auerbach, J.; Di Cioccio, V.; Saul, A., Production of a Shigella sonnei Vaccine Based on Generalized Modules for Membrane Antigens (GMMA), 1790GAHB. PloS one 2015, 10 (8), e0134478-e0134478.
36. Hartman, A. B.; Venkatesan, M. M., Construction of a stable attenuated Shigella sonnei DeltavirG vaccine strain, WRSS1, and protective efficacy and immunogenicity in the guinea pig keratoconjunctivitis model. Infection and immunity 1998, 66 (9), 4572-4576.
37. Cherepanov, P. P.; Wackernagel, W., Gene disruption in Escherichia coli: TcR and KmR cassettes with the option of Flp-catalyzed excision of the antibiotic-resistance determinant. Gene 1995, 158 (1), 9-14.
38. Datsenko, K. A.; Wanner, B. L., One-step inactivation of chromosomal genes in Escherichia coli K-12 using PCR products. Proc Natl Acad Sci USA 2000, 97 (12), 6640-5.
39. Liu, G.; Beaton, S. E.; Grieve, A. G.; Evans, R.; Rogers, M.; Strisovsky, K.; Armstrong, F. A.; Freeman, M.; Exley, R. M.; Tang, C. M., Bacterial rhomboid proteases mediate quality control of orphan membrane proteins. Embo j 2020, e102922.
40. SDS-PAGE sample loading buffer (4×). Cold Spring Harbor Protocols 2015.
41. Sansonetti, P. J.; Kopecko, D. J.; Formal, S. B., Shigella sonnei plasmids: evidence that a large plasmid is necessary for virulence. Infect Immun 1981, 34 (1), 75-83.
42. Qadri, F.; Hossain, S. A.; Ciznir, I.; Haider, K.; Ljungh, A.; Wadstrom, T.; Sack, D. A., Congo red binding and salt aggregation as indicators of virulence in Shigella species. Journal of clinical microbiology 1988, 26 (7), 1343-1348.
43. Angelini, M.; Stehling, E. G.; Moretti, M. L.; da Silveira, W. D., Molecular epidemiology of Shigella spp strains isolated in two different metropolitam areas of southeast Brazil. Braz J Microbiol 2009, 40 (3), 685-92.
44. Maté, M. J.; Vincentelli, R.; Foos, N.; Raoult, D.; Cambillau, C.; Ortiz-Lombardia, M., Crystal structure of the DNA-bound VapBC2 antitoxin/toxin pair from Rickettsia felis. Nucleic Acids Research 2012, 40 (7), 3245-3258.
45. Winther, K. S.; Gerdes, K., Regulation of enteric vapBC transcription: induction by VapC toxin dimer-breaking. Nucleic Acids Res 2012, 40 (10), 4347-57.
46. Anderson, C. Anderson series of promoters iGEM.
47. Livio, S.; Strockbine, N. A.; Panchalingam, S.; Tennant, S. M.; Barry, E. M.; Marohn, M. E.; Antonio, M.; Hossain, A.; Mandomando, I.; Ochieng, J. B.; Oundo, J. O.; Qureshi, S.; Ramamurthy, T.; Tamboura, B.; Adegbola, R. A.; Hossain, M. J.; Saha, D.; Sen, S.; Faruque, A. S. G.; Alonso, P. L.; Breiman, R. F.; Zaidi, A. K. M.; Sur, D.; Sow, S. O.; Berkeley, L. Y.; O'Reilly, C. E.; Mintz, E. D.; Biswas, K.; Cohen, D.; Farag, T. H.; Nasrin, D.; Wu, Y.; Blackwelder, W. C.; Kotloff, K. L.; Nataro, J. P.; Levine, M. M., Shigella isolates from the global enteric multicenter study inform vaccine development. Clinical infectious diseases: an official publication of the Infectious Diseases Society of America 2014, 59 (7), 933-941.
48. von Seidlein, L.; Kim, D. R.; Ali, M.; Lee, H.; Wang, X.; Thiem, V. D.; Canh, D. G.; Chaicumpa, W.; Agtini, M. D.; Hossain, A.; Bhutta, Z. A.; Mason, C.; Sethabutr, O.; Talukder, K.; Nair, G. B.; Deen, J. L.; Kotloff, K.; Clemens, J., A multicentre study of Shigella diarrhoea in six Asian countries: disease burden, clinical manifestations, and microbiology. PLoS medicine 2006, 3 (9), e353-e353.
49. Van Melderen, L.; Aertsen, A., Regulation and quality control by Lon-dependent proteolysis. Research in Microbiology 2009, 160 (9), 645-651.
50. Lupas, A. N.; Martin, J., AAA proteins. Curr Opin Struct Biol 2002, 12 (6), 746-53.
51. Melnikov, E. E.; Andrianova, A. G.; Morozkin, A. D.; Stepnov, A. A.; Makhovskaya, O. V.; Botos, I.; Gustchina, A.; Wlodawer, A.; Rotanova, T. V., Limited proteolysis of E. coli ATP-dependent protease Lon—a unified view of the subunit architecture and characterization of isolated enzyme fragments. Acta Biochim Pol 2008, 55 (2), 281-96.
52. Rasulova, F. S.; Dergousova, N. I.; Starkova, N. N.; Melnikov, E. E.; Rumsh, L. D.; Ginodman, L. M.; Rotanova, T. V., The isolated proteolytic domain of Escherichia coli ATP-dependent protease Lon exhibits the peptidase activity. FEBS Lett 1998, 432 (3), 179-81.
53. Hansen, S.; Vulid, M.; Min, J.; Yen, T.-J.; Schumacher, M. A.; Brennan, R. G.; Lewis, K., Regulation of the Escherichia coli HipBA Toxin-Antitoxin System by Proteolysis. PLOS ONE 2012, 7 (6), e39185.
54. Bolesch, D. G.; Keasling, J. D., Polyphosphate binding and chain length recognition of Escherichia coli exopolyphosphatase. J Biol Chem 2000, 275 (43), 33814-9.
55. Lorenzo-Orts, L.; Hohmann, U.; Zhu, J.; Hothorn, M., Molecular characterization of CHAD domains as inorganic polyphosphate-binding modules. Life Science Alliance 2019, 2 (3), e201900385.
56. Kopecko, D. J.; Washington, O.; Formal, S. B., Genetic and physical evidence for plasmid control of Shigella sonnei form I cell surface antigen. Infect Immun 1980, 29 (1), 207-14.
57. Zychlinsky, A.; Prevost, M. C.; Sansonetti, P. J., Shigella flexneri induces apoptosis in infected macrophages. Nature 1992, 358 (6382), 167-9.
58. Guyer, M. S.; Reed, R. R.; Steitz, J. A.; Low, K. B., Identification of a sex-factor-affinity site in E. coli as gamma delta. Cold Spring Harb Symp Quant Biol 1981, 45 Pt1, 135-40.
59. Miroux, B.; Walker, J. E., Over-production of proteins in Escherichia coli: mutant hosts that allow synthesis of some membrane proteins and globular proteins at high levels. J Mol Biol 1996, 260 (3), 289-98.
60. Hanahan, D., Studies on transformation of Escherichia coli with plasmids. J Mol Biol 1983, 166 (4), 557-80.
61. Wood, W. B., Host specificity of DNA produced by Escherichia coli: bacterial mutations affecting the restriction and modification of DNA. J Mol Biol 1966, 16 (1), 118-33.

### EXAMPLE 2—REDUCING THE EMERGENCE OF POTENTIAL ANTIBIOTIC RESISTANCE BY USING A TA SYSTEM TO AVOID PLASMID LOSS

Introduction

We have tested the effectiveness of a Toxin Antitoxin (TA) system, VapBC, to circumvent the need for antibiotics in growth media to prevent plasmid loss in bacterial cultures grown for protein expression.

Plasmids are mobile genetic elements that can both enter and also be lost from cells. In general, expression plasmids (those used for protein production in a heterologous bacterium such as E. coli) are present in multiple copies in the cell and impose a metabolic burden to bacteria, so the plasmids are readily lost. Bacteria that lose the plasmids are therefore at an advantage and can grow and reproduce more quickly and rapidly dominate a culture, which can be detrimental as it leads to a reduction of product (protein) yield. Currently, the standard method used to prevent plasmid loss is to use expression plasmids that confer antibiotic resistance to the bacteria that contain them. As a result, bacteria that lose these plasmids will die when grown in media that contain the appropriate antibiotics. A major problem of this system is that the disposal of antibiotic-containing waste products from large bacterial cultures, such as spent media generated by industrial biotech and pharmaceutical companies; the environmental discharge of antibiotics is known to contribute to the increase in antibiotic resistance of bacteria worldwide, and the FDA is urging companies to limit antibiotic use and release. New methods that can prevent plasmid loss are therefore needed to circumvent antibiotic use for this purpose.

TA systems are widespread genetic modules found on bacterial plasmids and chromosomes. These systems encode a stable toxin, capable of manipulating vital processes of bacterial cells, and a labile antitoxin that inhibits and even reverses these effects. TA systems prevent plasmid loss during cell division via post-segregational killing. In this process, if the daughter cell does not receive a TA system plasmid, the labile antitoxin is eventually degraded and the stable toxin expresses its toxicity, killing the cells that lack the plasmid. Meanwhile, the bacteria that do inherit the plasmid are unaffected by toxin activation because of de novo and continuous antitoxin synthesis. In order to evaluate if a TA system could be used to inhibit plasmid loss instead of the usual antibiotic resistance approach, we first tested the VapBC (virulence associated proteins B and C) TA system as a possible candidate. VapBC is the largest family of type II TA systems (characterised by both the toxin and antitoxin being proteins), where VapC encodes the toxin and VapB encodes the matching antitoxin. VapB is degraded by the Lon protease and therefore the VapB pool needs to be replenished constantly to keep VapC from exerting its toxic effects, which requires the use of Lon⁺ expression strain for this experiment. Most E. coli strains used for protein expression have been engineered to be deficient in certain proteases that might degrade the proteins one tries to express in them, which includes the Lon protease. For this study we used E. coli SHuffle T7, a strain suitable for T7 protein expression with enhanced capacity to correctly fold proteins with multiple disulphide bonds in the cytoplasm.

Methods and Materials

Cloning

The module of the VapBC TA system, containing the toxin, antitoxin and its promoter region, was cloned from Shigella sonnei CS14 and introduced into a pET28a vector previously modified to express protein tagged on its N-terminus by a 6-His tag, fused to an MBP tag, followed by a TEV cleavage site (the original pET28a plasmid only codes for an uncleavable N-terminal 6His-tag). The module was introduced between the T7 promoter and the lacI operon, so that on its 3′-end it lies downstream of the over expression cassette and is not affected by the T7 promoter, and on its 5′end it runs in an antisense fashion to the lacI operon and is therefore not affected by its lacI promoter. The resulting plasmid vector (pET28a-His-MBP-TEV-VapBC-CS14) is kanamycin resistant and can be modified for the expression of proteins like any other pET28a vector. In order to test if the vector worked in the absence of antibiotic or if it had any adverse effects on transgenic protein production, a protein gene coding for factor H-binding protein variant 1.1 (fHbp V1.1) from Neisseria meningitidis was inserted into the cloning cassette of the new vector (FIG. 1x).

Plasmid Loss Assay

The plasmid loss assay was carried out with E. coli SHuffle T7 cells, which are lon⁺ and spectinomycin resistant. The spectinomycin resistance is encoded in a plasmid that allows these cells to properly fold proteins with multiple disulphide bonds and, in order to avoid potential growth differences between the cultures due to the loss of this particular plasmid instead of our plasmids of interest, all cultures were grown in the presence of spectinomycin.

The cells were transformed with the following vectors (which confer kanamycin resistance):
Empty=pET28a-His-MBP-TEV
Empty+VapBC=pET28a-His-MBP-TEV-VapBC
V1.1=pET28a-His-MBP-TEV-V1.1
V1.1+VapBC=pET28a-His-MBP-TEV-V1.1-VapBC

Four 5 ml starter cultures of SHuffle T7 cells, each one transformed with one of the above plasmids, were grown in LB+spec+kan at 37° C. over night. 2 ml of each starter culture was then used to inoculate 100 ml of LB+spec. All four cultures were then grown at 37° C. until OD₆₀₀=0.8, at which point they were induced with IPTG (0.5 mM final concentration in each culture) and grown on at 25° C. over night. The cultures had the following OD₆₀₀ after 18 hours:

| | | | | Time to | |
|---|---|---|---|---|---|
| Plasmid | Cell type | Antibiotic | OD₆₀₀ | reach OD₆₀₀ | |
| V1.1 | SHuffle T7 | +spec | 0.795 | 4 | hours |
| V1.1 | SHuffle T7 | +kan + spec | 0.798 | 4.5 | hours |
| V1.1 + VapBC | SHuffle T7 | +spec | 0.781 | 4 | hours |
| V1.1 + VapBC | SHuffle T7 | +kan + spec | 0.793 | 4.5 | hours |

After equalising the OD₆₀₀ to 1.0, serial dilutions (1 in 10) were prepared for each culture and plated onto the following media then grown overnight at 37° C. in order to obtain some plates with sufficient single (as opposed to confluent) colonies that could be picked for the next step:
A) LB+spec (to select for all bacteria)
B) LB+spec+kan (to select for bacteria containing the expression plasmid)

Colonies were counted using the Promega Colony Counter app (FIG. 2x) on an android mobile phone. Comparing the number of colonies between plates with (B) and without (A) the antibiotic provided an estimate of plasmid loss, but this number is only a rough approximation.

100 colonies from each culture were picked from the LB+spec plates (those obtained from a dilution that produced single, non-confluent colonies). Each colony was streaked onto both an LB+spec and an LB+spec+kan plate, which were incubated at 37° C. over night, to check if the same colony could grow on both media.

Plasmid Loss was calculated by counting the colonies that grew on the LB+spec plates, subtracting the number of colonies that grew on the LB+spec+kan plates, and then expressing the result as a percentage (FIG. 3x).

Results demonstrate that in the absence of antibitoics, VapBC on the expression plasmid significantly prevented plasmid loss in the absence of antibiotics.

Results:

The plasmid loss assay reveals that there is a clear difference between growing cells with plasmid containing the VapBC TA system and growing cells with the same plasmids but without the VapBC TA system. While all of the colonies containing the empty plasmid with VapBC survived on the antibiotic-containing plates (0% plasmid loss), the majority of the colonies containing empty plasmid without VapBC died (86% plasmid loss). Similarly, most of the colonies containing the plasmid with V1.1 and without VapBC died (89.9%) but, interestingly, not all the colonies containing the plasmid with V1.1 and VapBC survived on the antibiotic plates (16% plasmid loss). It may be that expressing V1.1 provides a small amount of protection against the toxic effect of VapC.

In summary, the results clearly indicate that the VApBC TA system is an effective tool that helps prevent plasmid loss and in one of our experiments it actually managed to completely stop plasmid loss.

Sequences

All non-wild-type/modified sequences described herein are intended to be encompassed in the invention.

| Wild-type VapB sequence of Shigella sonnei: | | |
|---|---|---|
| (SEQ ID NO: 1) | | |
| METTVFLSNRSQAVRLPKAVALPENVKRVEVIAVGRTRIITPAGETWDEWFDGHSVSADF | | |
| MDNREQPGMQERESF | | |
| Wild-type VapB sequence of Shigella flexneri: | | |
| (SEQ ID NO: 2) | | |
| METTVFLSNRSQAVRLPKAVALPENVKRVEVIAVGRTRIITPAGETWDEWFDGHSVSTDF | | |
| MDNREQPGMQERESF | | |
| Modified VapB sequences based on Shigella sonnei | | |
| Modified residues are bold and underlined. | | |
| (SEQ ID NO: 3) | | |
| METTVFLSNRSLAVRLPKAVALPENVKRVEVIAVGRTRIITPAGETWDEWFDGHSVSADF | | |
| MDNREQPGMQERESF | | |
| (SEQ ID NO: 4) | | |
| METTVFLSNRSAAVRLPKAVALPENVKRVEVIAVGRTRIITPAGETWDEWFDGHSVSADF | | |
| MDNREQPGMQERESF | | |
| (SEQ ID NO: 5) | | |
| METTVALSNRSQAVRLPKAVALPENVKRVEVIAVGRTRIITPAGETWDEWFDGHSVSAD | | |
| FMDNREQPGMQERESF | | |
| (SEQ ID NO: 6) | | |
| METTVFLSNRSQAVRLPKAVALPENVKRVEVIAVGRTRIITPAGETWDEWFDGHSVSAD | | |
| AMDNREQPGMQERESF | | |
| (SEQ ID NO: 7) | | |
| METTVFLSNRSQAVRLPKAVALPENVKRVEVIAVGRTRIITPAGETWDEWADGHSVSAD | | |
| FMDNREQPGMQERESF | | |
| (SEQ ID NO: 8) | | |
| METTVALSNRSAAVRLPKAVALPENVKRVEVIAVGRTRIITPAGETWDEWFDGHSVSAD | | |
| FMDNREQPGMQERESF | | |
| (SEQ ID NO: 9) | | |
| METTVFLSNRSAAVRLPKAVALPENVKRVEVIAVGRTRIITPAGETWDEWADGHSVSAD | | |
| FMDNREQPGMQERESF | | |
| (SEQ ID NO: 10) | | |
| METTVFLSNRSAAVRLPKAVALPENVKRVEVIAVGRTRIITPAGETWDEWFDGHSVSAD | | |
| AMDNREQPGMQERESF | | |
| (SEQ ID NO: 11) | | |
| METTVALSNRSLAVRLPKAVALPENVKRVEVIAVGRTRIITPAGETWDEWFDGHSVSADF | | |
| MDNREQPGMQERESF | | |
| (SEQ ID NO: 12) | | |
| METTVFLSNRSLAVRLPKAVALPENVKRVEVIAVGRTRIITPAGETWDEWADGHSVSADF | | |
| MDNREQPGMQERESF | | |
| (SEQ ID NO: 13) | | |
| METTVFLSNRSLAVRLPKAVALPENVKRVEVIAVGRTRIITPAGETWDEWFDGHSVSADA | | |
| MDNREQPGMQERESF | | |
| Modified VapB sequences based on Shigella flexneri | | |
| Modified residues are bold and underlined. | | |
| (SEQ ID NO: 14) | | |
| METTVFLSNRSLAVRLPKAVALPENVKRVEVIAVGRTRIITPAGETWDEWFDGHSVSTDF | | |
| MDNREQPGMQERESF | | |
| (SEQ ID NO: 15) | | |
| METTVFLSNRSAAVRLPKAVALPENVKRVEVIAVGRTRIITPAGETWDEWFDGHSVSTDF | | |
| MDNREQPGMQERESF | | |
| (SEQ ID NO: 16) | | |
| METTVALSNRSQAVRLPKAVALPENVKRVEVIAVGRTRIITPAGETWDEWFDGHSVSTDF | | |
| MDNREQPGMQERESF | | |
| (SEQ ID NO: 17) | | |
| METTVFLSNRSQAVRLPKAVALPENVKRVEVIAVGRTRIITPAGETWDEWFDGHSVSTDA | | |
| MDNREQPGMQERESF | | |
| (SEQ ID NO: 18) | | |
| METTVFLSNRSQAVRLPKAVALPENVKRVEVIAVGRTRIITPAGETWDEWADGHSVSAD | | |
| FMDNREQPGMQERESF | | |
| (SEQ ID NO: 19) | | |
| METTVALSNRSAAVRLPKAVALPENVKRVEVIAVGRTRIITPAGETWDEWFDGHSVSTDF | | |
| MDNREQPGMQERESF | | |
| (SEQ ID NO: 20) | | |
| METTVFLSNRSAAVRLPKAVALPENVKRVEVIAVGRTRIITPAGETWDEWADGHSVSTDF | | |
| MDNREQPGMQERESF | | |
| (SEQ ID NO: 21) | | |
| METTVFLSNRSAAVRLPKAVALPENVKRVEVIAVGRTRIITPAGETWDEWFDGHSVSTDA | | |
| MDNREQPGMQERESF | | |
| (SEQ ID NO: 22) | | |
| METTVALSNRSLAVRLPKAVALPENVKRVEVIAVGRTRIITPAGETWDEWFDGHSVSTDF | | |
| MDNREQPGMQERESF | | |
| (SEQ ID NO: 23) | | |
| METTVFLSNRSLAVRLPKAVALPENVKRVEVIAVGRTRIITPAGETWDEWADGHSVSTDF | | |
| MDNREQPGMQERESF | | |
| (SEQ ID NO: 24) | | |
| METTVFLSNRSLAVRLPKAVALPENVKRVEVIAVGRTRIITPAGETWDEWFDGHSVSTDA | | |
| MDNREQPGMQERESF | | |
| Wild-type VapB homologues | | |
| Residues that may be modified according to the invention are | | |
| bold and underlined. | | |
| pDE105 | MRTVSIFKNGNNRAIRLPRDLDFD-GVSELEIVREGDSIILRPVRPTWGSFAQLDRADPD | 59 |
| pAA_O104 | MRTVSIFKNGNNRAIRLPRDLDFE-GVSELEIVREGDSIILRPVRPTWSSFAALEKADAD | 59 |
| pAA_Other | MRTVSIFKNGNNRAIRLPRDLDFE-GVSELEIVREGDSIILRPVRPTWSSFAALEKADAD | 59 |
| Salmonella_LT2 | MH-TTLFFSNRTQAVRLPKSISFPEDVKHVEIIAVGRSRIITPVGESWDSWFDGEGASTD | 59 |
| F-plasmid_K12 | ME-TTVFLSNRSQAVRLPKAVALPENVKRVEVIAVGRTRIITPAGETWDEWFDGNSVSAD | 59 |
| pINV_Sflex | ME-TTVFLSNRSQAVRLPKAVALPENVKRVEVIAVGRTRIITPAGETWDEWFDGHSVSTD | 59 |
| pINV_Sson | ME-TTVFLSNRSQAVRLPKAVALPENVKRVEVIAVGRTRIITPAGETWDEWFDGHSVSAD | 59 |
| PINV_096 | ME-TTVFLSNRSQAVRLPKAVALPENVKRVEVIAVGRTRIITPAGETWDEWFDGHSVSAD | 59 |
| | *. .::* ....:*:***: : : .*..:*:: * : *: *. :*..: . .. * | |

| | | |
|---|---|---|
| pDE105 | FMAEREDVVSDE-GRFEP* | 76 (SEQ ID NO: 25) |
| pAA_O104 | FMAEREDIVSDE-GREDL* | 76 (SEQ ID NO: 26) |
| pAA_Other | FMAEREDIVSDE-GRFDL* | 76 (SEQ ID NO: 27) |
| Salmonella_LT2 | FMSTREQPAVQEREGF*-- | 75 (SEQ ID NO: 28) |
| F-plasmid_K12 | FMDNREQPGMQERESF*-- | 75 (SEQ ID NO: 29) |
| pINV_Sflex | FMDNREQPGMQERESF*-- | 75 (SEQ ID NO: 30) |
| pINV_Sson | FMDNREQPGMQERESF*-- | 75 (SEQ ID NO: 31) |
| PINV_096 | FMDNREQPGMQERESF*-- | 75 (SEQ ID NO: 32) |

Plasmid Sequences

vapB open reading frame=underlined

vapC open reading frame=dotted underlined

overlapping nucleotide between vapB and vapC open reading frame=double underlined

vapB point mutation (T in CS14, A in 53G)=marked by \hat{ }\hat{ }

PINV_CS14 sequence
(SEQ ID NO: 33)
GTCCCTGATAGCACTGTTCCATCAGCGCTTTCAGGCGATAATAGTAGACCTTGGGTAGCCTCTGCCCAT
GTGTAACTTGATACCCCGAGAACAGCCACGATGATCTAAGCGGTTCTTATTTCTCCGGTCTCAGGGTCG
GTAATGCCAACAGTAAGTCCACAGTAATCAACGAAGAGTTTTTTGAACCGCCCCGGAAATCCTGGAGA
CTAAACGCCCTGAGAAAGAGGTAAACAGAATGACTAAAAATACACGTTTTTCCCCCGAAGTCCGTCAAC
GGGCAGTTCGTATGGTTCTGGAAAGTCAGGGCGAATATGACTCACAATGGGCGGCAATTTTTAGCCAT
GAAAACGTAATCAACTCGGTGAACCAATTCAAAAAATATACTTTATACCTGCGGAAGTGAGAAAACCAA
TATATTCAATACTTGCACTATTTTTAATGGTGCCAGATATATTGGTACTTTGCTCATAAAGAGATGTATCA
CCTTTTTTATTAGAAATGTAATTTCTGGAGCCTTCTATATAAAATTTTGCACTGGGGGTGACATAGTATC
CAATATTCGCAGCAACAGAGCAGTAATTTTGGTTTTTTATTTCATCTTTAAAAATTGTCTCAGTCTGATAG
TGTTTATCTGTATCAGATGAACGAACCCAACCACTATATTTCAGTTCTGCTCCAAACTCAAAATTCTCATG
GCGGTAATTAGCAGTTAATCCAATATAAGGTATTTTGAAGAGCTGTTTGTAATCTATTGTTCTTTCACCA
CTAGGATGTGCCCCTTTTTTATTCCTGTTCCCCCCGTTTTCACTATAAATATAACTCCCTCCCATTGCATTA
AAACTGTAACGACTCTCTTGGTAACCTGCTATTAGTCCAAGTCGATAATCCAAATTATTTAATAACCAAC
CTTTAAGATTCAGCTCGAATTCGTTAGCATCACGGAGATGCGTATTGGGATGCCAGCTCTGGTCGGTCC
ATACCTGAGGGGTATTGGAATTGTTCCAGTCTTTATCAACCATGCTTGCTTTCTGGTTACCTAAAGTAGT
CCAACCGGAAACTCCGAAAGACACTTTTGGAATTAGCTTCCAATCGATGCCACCTCTAACAATAGTCGC
ATTACTGTATTTCCAGTCCAGTTGACTAATTTTTCTCCCCCCTTCCTTGGGATGATAAACGCGTTCTTTTG
TTTTGCCACTCAGAGATCCTAGACTAATATCCGTACTGATGTTGTCCGGTATGAAAAGTGGATAGTTAG
TGGTAGCATGTGTAGTAAAGATCGCTGGTACACAAAGTGCAAGTACAAGGAATTTTAATTTCATCGAG
AACTCTACTTTTTTGGTTGAAATGTCCATGATCTTCTTACTTTATAAGATAAACGTCAATGCCAAGCATTA
TTTTAATTAAATATTTGTTTATGTTACCAAGAACATATCTTTTATTATAATATTTATTGAACTTTTGTAAAA
ATAAAAAAAATACCTCATTGTGCGAATAAAGTAACGGGGGCCATAGATTTCTCTACCTACCTTTTGGTG
TTCATACCTGAGGCTGATAGTTTCAGGATTCCGCGACAATTCTACTCACCTTCTTGTTCTCTCGCCAATAT
GAAATCAATCAGAAAAGCGTTCCATAAGAACAACGATAAGACAATAAATTGTCATCACGGCTGCAATA
GCCAACGTGCCTTTCAGTACCAACATGACAACCTCCTGTATTTGACGTAACGTGTGCTGATAAATATGTA
TTTGTATCTCAACTGACTCAATACAACACTTAAGTATAGTTTTTCAGATTTTGTTTTTCCCGTGAAATATT
ATGGAAATGGCAGAAGCAATACATTGAATTCCGATTCGAGTGAATACGGCTAGCGGGCACCTCACTTT
AGCACTGAAGCCATCAACTITTATTTGACTTATTTCAATGGAAAGCTTTCTCACTATCAGTACAGGTAGT
TCCCATCCTTTTAAAGTCGAGAGTCAGACTGCGACTAATACAGTTAAAGTGACCAAACTCAGTTATCCTC
TACACATTTTACCTCATGTTTACTTGGACAATTCATTGGTGCTTTTATAAAAGAATAAATATTCTACTAAT
TTAATGCATCACCTAAAATAGTATTTTTTATAATAGAACTGTATCTATAGTATATTATTAAAGCTTAATTA
TTTATTATGCTTTAATGTGGTTGTCCGATTAAGGACTAATCCTTGAAAACGCCGTATAGGGCGGGAGTT
CACTCTATTGCGATAACGTAACCGTACAATATTGTGCGTTTCCCAAATTGACCACTACTGAACTATTCTG
GGATTTAAATGCAATAAAATGGTTGGTTGAAGGTCGTGGTGATGATGTTAGTCCTTTTGAGAATATTCC
AGTGCCAGATCTTACTTATAATAAAGAGGCTTGGCAGTTTGGATAAGCTATTGTCCTTATTCCACTAATA
CAAACTTTTTGTAAAGTATATTAAATATACTTCATTTCATACTGAAATCCCCGGAAGTATTCAATCAAACA
ATTACACCATAAAAGGAAGAGGGAGCCCCAATTAAGTTATCAAGCATGTTTATGATACCTGCAGGGTAT
AAATTTTCTGGCTGCAAGCAGACACATATAAAACGTCTTTATTGATCCTACCCACATAATATGGACGCAG
TCGTAAGTGAGGCTCTGTGAGCCTACCCACGTAATATGGACACAGGCCTAAGCGAGGTTCTGGTTTTCA
AATTGTTCCGGACTGAGGCCGCCACACCAACGGTGCCGTCGCCAGCGATTGTAATCGCACTCGATATAA
TTAAACACTGTTGCCCGCACTATTTCCCGATAGATAAAGTGTTCTCCGTGGAGACATTCCACTTAGGAAC
AATTATATTGTTCATGGTAATGTTTATATTTGAGTATAGAGATTAAAATGATTAGTCCCATCAAGAATAT
TAAGAACGTATTTCCAATAAATACTGCAAATACTGAATATATAGTGAGAAATATTTATCCTAGAGTTGAA
CATGGTTACTTTAATGAATCACCAAACATATATGGCAAGAAGTATATCTCTGGTATAACCAGAAGTATG
GCACAATTAAAAATAGAGGAGTTTATTAATGAGAAATCCAGACGACTTAATTATATGAAGACAATGTAT
TCTCCATGTCCGGAAGATTTTCAGCCTATTTCCAGAGACGAAGCCAGTACACCGGAAGGAAGCTGGTTG
ACAGTTATATCTGGAAAACGTCCTATGGGGCAATTTTCTGTGGATAGTTTGTACCATCCTGACTTACATG
CATTATGTGAGCTCCCAGAGATTAGTTGTAAGATCTTCCCTAAAGAAAATAGTGATTTTTTGTATATAAT
TGTTGTATTCAGAAATGATTCTCCCCAAGGAGAACTACGGGCAAATCGGTTTATAGAACTATATGATAT
AAAAAGAGAGATTATGCAAGTGTTAAGAGATGAGTCGCCAGAGTTAAAGTCTATTAAATCTGAGATTA
TTATTGCTCGTGAAATGGGAGAACTTTTTAGTTATGCATCAGAGGAAATAGATAGCTACATAAAACAAA
TGAATGACAGGCTTTCTCAAATCAAGGCTCGAATGCCTGTTACTTAATGTATATTAATTGTAWRMRRW
KSWMMMMMMMMMMWRRMMRMMRMRWWMRMSWWWWWMSRRWMWMRWYWWWM
YRMWWMKWWWRRSRRWMMMMMMMMRWWMWMWWMRWRMRRYMWWMKYRYRSWK
WMMYMWSMMMYRMWWWMRYMMRWWMKRRMSWRMRMWRMMWMKMWRMMRMWW
MMRWMMYMSMMMRYMRRMMWMMMWWMRWWMWWMMKMMWMMWRMMRRMKMS
WWMSRWWRRRMYRWWMWMMMMRMWRWYWMYRMRMMRRYWMWWMSWMWKYSWR
MWMWSRWWYMRMMMMMMYMRMYSMYRRMMMWKMMWSMYMRWRWMMWKMMWRM
MMWRMWMKMKKYRRMMYMWMMMMMSRMMRRRYWWMMMWMRRRWKWYMSWWRMM
RWWWMMMRMRMYWWMMWRRWRRRMMWRMKKWMMRRMRWRMMMRMMMWSRMM
MMRMMMMMWRRWWWWMWWRYRMMMRRRYYRRRMRMMMMRRMRMRRYMMRSMYMR
MRMWYMMMMRKMMMRMYMWMRSYMMYMWMRMMRMMMMMMMMMWRMWWKRRM
WMSRWMMMRKMRMMMWRMMKWWMMMRRKMRWWMRRRMWRRMMMMMWRWWMM
WRMMMMMMRYSKWWMWRMMRRYRMRWSRRMWRYWRMMMRMWRMMMMRMMMMM
RMWMWWWMMWRMRMMKKMMMSMMMRMMRRSRWMMMMWMKRKWMRSMKSWMMR
RRWWKSMMWKRWRRMRMWRMWWSWWRMWMMRKMMRMRMMRWRRMWRMWSMMMY
RWMRYWYMMKWMMMWKMMWKYSWMMWMYMRMMMRKMWRMMRYMWRRWWYWWM
RRRMMRRWWWWWMMMRWMWYWKWMRMWRSYRMRMWRMMMMMMCGAGTAGGCAGC
CTGACGGCTGCGGCTTGTCATGGTCTGCAATTACCGTTATAAAAACAGGCAATGTCATTGTCTTTCAGG
TGGTTATATGGCCCGTTCAGCTAAACCCCGTAAACGTAAACCCGCCTCTCAAAGAAGCAAACTTCCCCG
CTATGTCGTGAAGCTTCACGAAGATGATTTTTTTGACGAAGAAGACGCAGAAGTTCTGCGCTTTGATAG
TTTTGACGATGCMRTTGAMWRMWRMRMMRMMMWRMMWMWKMMRWWCWWWRTGGATG
CGGGAAACAAAAAGCTGGTCTTCTGGTTTGTACGTGTTGATGACGAAGGGTATCCGGAAATCGCCCGC
TGCACGGAGCGGGAATTTGCGACCATTCTTTCCGGTATCAGCGCCGGCGGTATGTACTGCCCGGAGTG
TGGCACGGTTCACTGGCCGGACGGAGTCGCCCCGCCCTTCTGATGCTTCCCCGTTTTGCCGATATCTTTC
AGCAGGGAAACCGCTGGCTTAACTGGCTGGAGAAGCAGCCGGAARGGGCAGTACGTCCGGTGGTGAT
TGAGTCGGTGACCAAAATCATGGCGTGCGGGACCACACTGATGGGGTATACACAGTGGTGCTGTTCAT
CACCGGACTGTTGCCATACAAAAAAGGTCTGCTTCCGGTGTAAAAGCCGCTCCTGCCCGCACTGCGGG
GTGAAGGCTGGCGCACAGTGGATACAGTATCTGCTGAGCCTGGTCCCCGACTGCCCGTGGCAGCATAT
TGTGTTCACACTTCCCTGCCAGTACTGGTCCCTGGTGTTCCACAACCGGTGGTTACTGACAGAGATGAG
CCGCATTGCAGCGGATGTGATACTGGAAATCTGCCATCAGGCAGATGTGGAGCCGGGGATATTCACGG
TGATCCACACATGGGGGCGTGACCAGCAGTGGCATCCGCATATCCATTTATCGACAACTGCCGGTGGT
GTGACGTCGGGCCACACCTGGAAAAATCTTCATTTTTACGCCCGTAAGGTGATGAGCATGTGGCGCTAC
CGGATAACACGGTTGCTGTCACGGAAATACCCTGACCTGGTGATGCCGGATGCGCTGGCAGCAGAAG
GAAGCAGTAAACGGGAATGGAATCGCTTCCTGGACACGCATTACCGCCGTGGCTGGAATGTCAACGTA
TCCCGGGTGATGGATAATGCCACCCATGTGGCGGTGTACTTTGGCTCTTACCTGAAAAAGCCACCAGTG
CCGATGAGTCGTCTGGAGCATTATGCCGGTCAGGATGAAATCGGTCTGCGTTACAACAGTCACCGAAC
AAAACGGGAAGAATACCTGTTGATGAGTGGAGATGAGTTCATGGAAAGGTTCTCCTGGCATGTGGCG
GATAAGGGGTTCCGTATGGTGAGGTACTACGGTTTTTTGAGTCCGGCGAAACGGCGGTTACTGGAAGA
AGTGGTGTACATCATAACGGAGACAGTGAGAAAAACGGCGATGCAAATCACCTGGAGAGGGATGTAT
CAGAGGTTACTGAAGGTTGACCCGCTGAAGTGCGTGCTGTGCGGGAGTCAGATGCGTTTTACGGGGCT
GAAGCGGGGCTACCGTCTGGCAGAGCAGGTCCTGATGCATGAGCTGCTGGCCCGGATGCGGTGGTGC
GGCTGAGAGCCGCAGAGGGGAAGTTGCGTCCATTTTGAGGGAAACGGAGCAAAAAACCGCCATTCAT
ACCCTGAATCAATCAGAGCCATCCAGTTTAATCGTCGGTTCCGTTCATATGGAGCAAAAGTGGTGTTGA
AGAAACATCCGTTTTGTGGTGTTTTTTTAATCTTTTTGGGGTTTTAATTCCTATCGATGAACGCAACCTGA
CAAGAGAGAGAACTCAGGTCGGGCTTCAAGCAGCACGAGCCAGAGGACGTAAGGGCGGCAGGCCGA
AAACACTGAGTAAGGATAAACAGGCCCTTGCTGTTCAACTCTATAACGAGAAAAAACATACAGTTGCGC
AGATTTGTGTGTTGATGGGGATATCGAGGCCAACGCTGTACAAATACATAGAGTCAGCCAGGTTGTTC
AAGAAATGAACTTAGCGTACGTTTTCGTACGGTTGGGCTTCAAATCCCTAATACGTTGACGGGGCAATA
TGCAGTTCGCTGCATAGCGGTCCGACCCCGTACTGCTCACGCAGCTTATCCAGCAGTGGCATCATTTTTT
CCAGAGGCGGTCGAACTCCGCCTTCGCAAAATAAGCGGAAGCTTGGCGAAGGATATCGTTACTGCGGC
GCAGTTCACGATTTTCACGCTCCAGCTCTTTCAGACGCTGACGTTCAGCGGTGGTGAGCCCTCCATCACC
GCCCCCGGTATCCCGCTCATGCTGGCGAACCCGGACACGCAGAGTCTCCGGCGTACAGCCAATCTTTGG
AGCAATGGAACAAATTGTCGCCCATTGTGAGTCATATTCGCCCTGACTTTCCAGAACCATACGGACTGC
CCGTTGACGGACTTCGGGGGAAAAACGAGTATTTTTAGTCATCCTGTTTACCTCTTTCTCAGGAAGTTTA
GTCTCCAGGATTCCCGGGGCGGTTCATTTGAGGATCGCCATGATTTGGCTGTCGGTAAATTTTGATGTT
TTCATAAAGAATCTCCTGCGTTTATGGTACGAGAAAATTCTACTTTTGACGCTGGTTAATTTACGGGGTG
ATTACCAGACCAATCGCCAGTGGCGCAAAGCCCGCAGGAGCGCGTTTTTCTGTCGCTCCGGACAAAAA
ACGCACTGAGGACAAACTCGCTCAGCATACAGGCGCTTAACGCATAGCCGCCAGGTGAATGGAGGCCA
TAACCATTAGCCGCAAAACCGCTGGTTGTCGCATCAAACCCAGCCTTACCGCTGGCGATACCATACAGC
ACTGCCGCAGCGGCAATACCGCCGATAACCTGAGCGATAATGTAAGGTAACACGCGCGCTGCTGGGAA
GCGTCCACCGGCCCACAGACCCAAGGTCACCGCGGGGTTAAAATGCGCACCAGAGATGTGCCCGACCG
CATAAGCCATGGTGACTACTGTCAGACCAAACGCCAACGCCACGCCCAAAAAGCCTATACCTAACTGTG
GGAAAGCAGCAGAGATCAAGGCGCTACCACAGCCACCCAGAACCAGCCAGAAAGTGCCGAAAAATTC
GGCGCTAAAAGGTTTAAACATACACTGATGTCCTGTTTGTTAAAGAGCAGGCATCAGGGTAAAGGCGG
TCACGATGTACCGCCTAGCGGGGATTTGTAAGAGAGGTGAAGAATTGGTGTGGAAGGTTAAACCTAA
GCAATCACAGGCGAATGACATTGCTCGCTTGTGATAACAGCGATTTTTTCTGCTGCCAAACTAGATCGC
AAATATCATCAGTTGGATTGAATGCCGCTGGTGCTTGTAACAACAAACTGCGAATTCGCTCATGATCAA
AGTCATGACAAGCTTGATCCAGCTCTTTAAGTAAGAGCGATAGATCCTGCCACGGTAGCATCACTTCGT
TGGCCGTCATAATTCGTGGATGAGAGGTACCTTGAACTGAATCACCAATCAGTAATTCTTCATACAGTTT
CTCACCTGGACGTAATCCCGTAACTTCAATGGCAATATCGCCATCTGGATTTTTATCATCCCGTACACTC
AAGCCACTTAACCGGATCATGCGTTTGGCTAAGTCATAAATCTTGACCGGATCGCCCATGTCTAAGACA
AAGACATCGCCGCCATGCCCCATCGCCCCCGCTTGAATCACCAACTGTGATGCTTCCGGAATTGTCATG
AAATAGCGAATAATGTCACGATGAGTCAAGGTAACTGGCCCACCTTGGGCAATCTGTTTTTCAAACAAC
GGCACGACAGAGCCCGAAGAACCGAGTACATTACCAAATCGCACCATACAAAAGCGAGTTTGGCTTTG
CCGTGCAGACAAAGCCTGCAATACCAATTCGGCCAGACGCTTAGTTGTCCCCATAGTGTTGGTCGGGCG
CACCGCTTTATCGGTGGAAATCAACACAAAAGTTTCAACGCCACTTTCGATCGCTGACTCAGCGCAGTA
CAAGGTACCAAACACGTTATTACGTACCCCTTCCACCACATTATGCTCAACCAGAGGCACATGTTTATAA
GCGGCCGCATGATAAACCGTTTGAATACCAAAGGACTTCATCACCATCTGTAAGCGATTCTGACGCTGC
ACCGAGCCCAACAGAGGGATCACTGGAACATTCAAAACTTCTTTGTCACACAGCGCCGAGAGCTCTTTC
TCAATAGCGTAGAGGGCATATTCAGACAGTTCAAATAGAACCAATTTGGCCGGCTTATATCGAACAATT
TGACGACAAAGCTCAGAGCCGATCGAGCCTCCCGCCCCAGTGACCATAACGGCTTTGCCAGTAATGTTT
TCGGCCATCAATTTGGCATCAGGAGCAACCGGATCACGACCTAGTAAGTCATCAATCGATACTTTTTTTA
GATTACTGATTTGTGCTCGACCTTCGACTAAATCGACCATACCCGGAATAGAGAGAACTTCACACGGTA
GATGCTCTAAACGAGTAATCACTTTCTGCCTTTGTGACTTACTGACGCTCGGCATCGCCAAAAGAATTTT
CTTTATACCATAGCGATCTACAAGGTAACTCAGTTTATCCGAGGGATAAACTGTTACACCATGAATGAC
AGCCTTATGCAAGCGAGGATTATCATCCACAAATGCCACAGGAAAATATTCTTTTGCCTGCATTAATGCT
GGCAATAATTGTCGGCCAGATTCACCAGCGCCATAAATCAAAACAGGCGCACTACCCTTAACTCCATAG
TTAAGTATCATTCTAAAAAGCAATCTAGAGCCTGATGTCAGTAAAAGTAAAACCAAAAAATAAATTCCG
ACAACAGTGCGCGGCAACATTATGGATAGCGAATATGATATAACGACAAGAGATAACGTTGAGGCCAG
AATACCAACTGGTATTAGCAACATTATCTTTGCACTAACATACCGTAAGACTGCCCGATAAAGCCCAATC
CTGATAAATATAAACACGGTAAATATGGTAAGAATAGCTGCAGCAAACCACATCGGTGCACTAAGAAA
AGCCGTTTGCTCATCAAGCCTCAACCAATAAGACAACCAAAATGAGAATATCACCATAACTACATCAAT
GCAGATGATGATACCTCTCTTAACAATTCTTGGCAGCTCCAATATTCTATCAATCATCCTACACTACCTTA
TTATCGCGACAAAATCTTAATAATAGTTTTCCAAATTATCACACAATCATACTTTACTGAATTGTTAGCAA
CATAATCTAAATAATATCTTTGCTTGATTGGAAGAATTATATCTATATAAGCCCTACGTGGGTCATCATA
ACTAGACAAAATCTCATTTTCATCTACCATTTCTATAGATGCTAAGTCAGTTATCCCTGGCCTAACCGATA
AAACTTTTTCCCTTATATCATCAGGATACTCATCAATAAACTCCCTCACTTCCGGTCTTGGGCCAACCAAA
CTCATTGTTCCAGAAAGAACATCTATCAATTGAGGAAGCTCATCGATTTTGTACTTCCGTAAATACCATC
CAACTCTGGTTACCCGAGCATCTTGACCAACTGTTATCCGTCCTTTTTTTTCTGAATCGATAAACATCGTT
CTAAATTTATGTATCCTAAAATCTTTCCCAAATCTCCCAACTCTATACTGCCTAAAAAAAACCCCCCCTTT
AGAATCAGCAACAATTAACATTGACACAATGATAAAAACAGGAAATAGAAAAAGCAGGCCTAAGCCTG
CCACGATAACATCAAAAATACGTTTCATTTAATATAAAATACCATTACATAAATAATTGTCTAATCGATTT
GATAACGAACAACTGATCTTGATCCGTCATTTTAGTATAAAGAGGAATAGATATTTCATTTAAATAACAC
TCCTCAGAAACTGGAAAGTCACTGGCGTTCAAATTATATGTATCACGCCAAACCGGTTGCTTATGCAAC
GGTATAAAATGGACAGAACAACCAATTCCAAGATCTGATAACTTCTTAATAAAATCATCGCGATTAATA
GCCGAATCAGTTTTTAAGCGGATAGGATATAGATGCCAAGCATGAATATCACTAGCATTAGTAGGCCAT
TCAGGCAATTCAAGTGGCAATTCTTTTAACGCATCATCGTAAATTTTTGCCATTCGTTGACGTTTTTTCTG
AAAATCATCGATCTTTCTAAGTTGATGAATACCGATTGCCGCACAGATATCAGGCATATTGTATTTAAAC
CCTGGAGCTACAACCTCATAAAACCAAGAAGGAGTTTTAGACTGGTACCGGTCAAAAGCGTCACGACT
GATTCCATGTAAACGCATTACCTTACAACGCTCAATTATATCTTTATTTTTTGAAACAACCATTCCGCCTT
CACCGGTTGTCATAGTTTTATTGGCGTAGAAGCTAAAAACCGTAGCATCTGAATCAAGCGTTCCTATCTT
ACTTCCTTTATATGTTGTAGGAAATGCATGAGCGGCATCCTCGACAACCTTTAGGTCATATTTTTTAGCA
ATTGATAAGATTGAATCCATGTCACAAGCTAATCCAGCGAAGTGTACTGGAATAATCGCCTTTGTTTCAT
TAGTAATAGCCTTCTCAATGGCATCAACTGATATATTAAATGTTTTACGATCTACATCAACAATTACAGG
ATCAGCACCAAGGTACCTGACAATTTCGGCAGTAGCAGTGAATGTATATGATGGGACAATAACTTGGT
CTCCCGGCTTTACGCCAACAGCTTCAAGAGCCAAATGTAAGCCCGACGTAGCAGAGTTAACAGCTAATG
ATTGAACGTTCGCTCCTAGGTAATTAGAAAATTCTTGTTCAAATTGCTTAGCCTTAGGACCTGTCGTAAT
CCAACCTGAACGTAAAGAGTCAATTACCTCTGCAATTTCTTCTTCGCCAATTTCAGGTAACGCAAAAGGA
ATAAAATTTTTCATTTAAAATACCTTTTCAAAAAAGATAATAACGATCCACATCCATAAGAAATATGATA
AGACAAAAATATAAAAGGAAGAAATACTCTACCTATAAAACTTCGCCCATCTCGAATTGAAAACAAAAT
AGAAATAAGAAAATAAAAAAATAAAAAAGATAAATATATATAATCACCAGTAGCAAAGAAAAGAGAA
AAAGATACAATTAAATACAAAACAAAAAAAAGTGGCACTATATGTCTAAAATAATATGCTCCAGACATA
ACAACCCAAAAACCATCAAGAAAACCTTTCTTTATGAAATCTTTAAAATTATCTCTAACATAATACTCAAC
AAATAAACTATTATCTGTATATAATGACAAATTATTGCTTAAAACTCTCTTATGAAAATCAATATCTTGAT
TTCTATCTAATACTTCATTAAAAAGTCCAACATCAAAAAACACATCTTTATTATATAAAGCAAAGACAGC
TGTATCTGTTTTTTTTAGTCGATTATTTTTGTCTACGCACCTAAATGGAGAATTACCAACACCAAATTTCG
ATGCATAAAAATCCGCTACAATTGAACCAAACTTAGTTTTATAAGAATTAGTTAATACACCTCCAACACC
AACAACGCTATCATCAAACTGCATAATATTAAAATAATCATCTAATAATTTAGATATATAGTTATTTGGA
ATATCACTATGTGCATCAACTCTACACACAAACTTACCATTAGCTTCTAGCACCCCAATATTCCAACCCGT
AGCAAGAGTTCTTTTTTTATTATTGATAATTTTATGCTTAATGTTGCTATGTTTAGAAAAAACATCCTGAA
CAATAGATATTGTATTATCAGATGAATCCCCATCTACAATAATCAATTCAAATTTATCTAATCCAACATCT
TGATTAATAAATGCCAAAAGAGATTTTTCAATATACTTCTCTGCATTAAAGCAAACTATAATTAAAGATA
CTAGAACATCACTCACAAATACCACTCCTCATAAAATATAGTTTTCTTTTCCCTGAGTGAACTGAATCAAC
AAACAAAAATCTATTATCAACCGAGATCCTTGGATGTAAGTCACATCGAGAATAAGAAAAAAAACTCAT
TGACTCATAAAATAATCCAAGCTCGCGATAATCATTTTTGACAAGGTCATAAACAAACAACTTTTTAATT
CTATTTTTATCTGGGTAAGTATCAGATATAATATATCTATTATGAACTATAGTAGGATGTCCATCAGAAT
ATTTTCTTGCATCAAAAAACAATTTCTCTGTATTACAGGATTCAATTGACAATCTATAATAATTTAGCGAA
TCTATTTCTGCACCAAAAAAACCTATAATTTCATCATTCCCATTCCAACAACAATGACTAATAATCGATTC
ATTTATTAGGACTCGAACATTATCATTTATTAAATTCCAATGTATTAACCTTTCACGCTTTCCTTTAGGTG
TATAGTATCGATGTATGAACATAACGGAACGTCCATCGGGAGCAAACATAAAATGATTGACCACATGTT
CAACATCAATATTACATTTAAAATTAGTTCTCTTTAGCATTTCGTCTAAGGATAAAAATAATTCTATTCCC
CCCGTATTCAAATTAATGATTGAAATATCTGTAGAAAATGATTTTTTATTATAATAACCATAATCGCTATC
ATATTTAGCCAGCCATGAGAAATCTAACGTCACAGCCTTGTAATTATTCACATCATAAATCGGATAGTTT
ATTTTTTTTATAACCTTCATCAAAGACAAAGAATAGACAACACTAATGTATCCATTATTTTCATAGTCATT
AAAAATTATATTGTCATCATCAATCCACATTAATCGGCTTCCCTGCTGCCAATTAAATGCTCGCGTATCAT
ATAGCTTATAAACTTTGTTATTAAGAAGGTCTTTTACGCAAATAGATATATACTTAACTTTCTTTGGATGT
CGTTTAGTGCTAAACGCACTAGAGTGGAACAATACGTACCGCCCATTAGAGCTCATTGGACTATGGTCA
TAATAGCCAAAAAATGATTCTTCAGAATCATCAATTGAAATCTCGTATATTTTTGATTGAATAACCGTCTT
ATTTTTATTCAAAATAATTCCGAATAATGATGCGATATATAAATAAAAGAACTTAATAACATCCCTTACCT
TAGGATACTTACTCAAGGCTAAGGAAATTTTTCTTTCAACATATTCTAACAACATATTCTCATCCTAAATA
GCTGCGCTTAATACCGCTACACTTTTGCCAGCCCATGTTTGCCTCCGGGGAATGGGCTGACGGTTTCCA
TAAAATGGCGAACTTTTTTCAACAGTTGCCACATTGAGCTGCACTGATGATTACGCGTTATTGTGTCGTG
AAGTGCCTGCCATAGCCGTTCAACATGATTCATCCATGGCGAGTAAACCGGCTGATAAATGACCCTGAA
CTTCGGGTTCTCCTTCAGCCAGCTCTGTGTTTCCCGGCTTTTGTGGATAATGTAGTTGTCCACGATCAGC
GTGATGGTTTTCGCCCGACGGTATGTCGCTTTAAGCCGCTTCAGCAGGCTGATGAACAGCGCCGAACTT
TTGCTGTTGCCGCCCACACAGCTGACTTTACCTGTCCCGCTGTGCAGCGCTCCGGCCAGATAATATTTTT
CATTCTGTCCCGGCGTGACCACCCGTTTTTGCTGTCCGCGCAGTTGCCAGTCCGCACCGATTTTGGGATT
AAGATGGATATCCACTTCATCTTCATAAAAGACCGGATGCTCTGCGCTGCATTCGTCCAGTGCTTTATGG
ATTGCTGCCATCTTTTCATCTTTATGCGGGTCACGGATACGCAGAGTTGGCGCAGCCCTTCGCCACACAA
TCCCCGCAGACGGCAACCAGCGGCGAACGGTTCCGGCATTTAACTGGCAACCGGTTATCTCATTGATTT
TTATTGCCAGCAGTTCTGTACTCCAGCGTGAACGCTGGTAGCCAAAGTCGCCGGGAGAATGTTTTACCA
GCTCACGTAACAGTGTGCAGATATGCTCAAACGGCCAGCGACGGGCACGCCCGGCAGGTAATGATTTC
AGTCCCTCAACACCCGACTGCGTGAACCAGTTAATCCAGCGTCCAACAGAGGAACGGGCGCAGCAGAG
CGTTCTGGCAACGTCGCTGACACGGTCGCCCCGGTGCAGCATCAGCATGGCAGTCAGTCTGCGGGCAT
AATTTTTATCGTGTGTTTTATGGATGGCTTTCTGCATCAGGCGTCGTTCGTCACGGGAAATTGGTGCTAT
GATCGGCATTGCTCAGTCCGGTTGGTGATTTGTTTTGATTTGGCGATTGATCAGATCGCACAATCCGGG
CTAAGTTCCCTCAAAGTGATCTACTATTCCGCGCAGCTATTTAGAATTACGTATAAAAACCACCGATAAA
AAAATAGAAATATAAATATATATCGCTGGCGTCAATATATACTCATTAAAAAAGACATAGAAAGCCAAA
GCAAAAACGCATCCTTTACCAATATACCCTCCGCATTTAAAAATTGGAGACAGCATTATATAAAATACAG
AAATAAGCAAAATAAAACCAATAATCCCTAACTGAGCCGAATAAGATAAATAAAAATTATGCGCCAACC
CAAAGTTGGTACTTCCTAAAAACTTTTTATCTATAGCCTTTACATAATTTGCAACACCTAATCCAGTGCCA
ATTAAATAATTTTCTTTTAAAACATCAACCCCAATAAATACTAACTCAACTCGACGATAATCATTCTGACT
TTTTGTAAGAGTGTTACCACCAGAGAATATAACCTGTGACGAAGACAGCATTCGTTCAAGACGAGTGCC
TATATCAGTAAAAGAAATACCTAAAGTTAGAAGAGGAACAAAAAACAATGATATTAATAACTTTTCCTT
CTTTGTTAACTGAAGAAAAACAAAAAACAACGATATAGCAAATGATATTATTCCTGCTCGGCTATCCAG
AAAAAGAAAGAACAGAGAGTACAATACAGATGAGACATACCAAGACTTTCGACCACGATAAAAGTATG
AATATACACAATAAAAGCACACTACTATAATTGCGGTGGCATTTCTATTTAAATAAACTATAAAATTACT
ATCTTCAGAATAACCAATAAATAATTTTACAACTCCACAGATAATACCAAAACACATTACAACATTTATA
TTTCTAACCGAATGAGTTAAATAAAGCTCATCTTTACCAATGCAAAATGACACCAAAAGAACCAAAGCT
ATAAAATTAGAAAAAAACGATAATAAAATTACGACACCTGATATATCCGCTGATAACAACAACATCTGA
ATGCTGATATAAATCAGGAATATGATGCCTAAAAATAATAATTTCTTTGTCCACAGAGAAACGTGAAAC
CTCAACGTTATTATAGATGTTAAAACAACAAAGAAATAAAATATTAGATCTGGAATATAGCCAATAGGA
ACTCCAAAAAAACGCTCTAAATCAGAGTTCATTTGTCCATAAAAAAAAACCGGAAGGATATTAAATAAC
AAAAGTACAGGATATAGGTATATTAACATACTTAACCTTTATTAGGCTTCCAATCGAGTCTAAACATATT
AATGGAACGATATAAAACCACAACATATTAATATAACAACCGCTGCTGACCATTAATAATAAAATAAAA
GAAAATTGGGACAAAGATCATCATTAGTGAATACTTATCATGAAGAATTTTCCCACAATCTTTTTTCACA
GAAATAATATAAAATATTCCAATTACTAATGTTGCAATGGCACTTCCAAAAACCGCCCCACTCCATCCAT
ACCGAGGTATAAAATAAAAGAAAACCGCCATTGAAATGACCACACCAATACAATACGATTTAACTCGCT
CGCTTTGATTTCCACTACACACAAGATAAAGTTCATTTATTAGCCAAAAATTATATTTAATAAGAGCCAT
AAACATTATCAAAAATGTTGCAGAAGATATTACCTTATACTCATCACCAAACAATATCCCTACGAAATAG
ATGCCAAAAAAATAATAGCATAAACATAGTAATAAGACTAACAGTGATATATATCGTTGTAATTTCCTTA
TATTACCCCATTCAGAATTTTTCAGATAATAAGAAAATGAAGGCAATAAAACAGTATAAATAACATTAA
ACACTATTATTAGACCGGTATTAACCCTAGTTACCACCTGATATATAGCAACACTATTATAAGAGAAATA
TTCTTTTAATATAAAAAGGTCAATACTAGATAAAACAACATTTAGTAACACCAGAATAGCGAACGGAGT
TGCATCCAAAAAATCTTTATAAACTAAAATACTTGGTCTTTTTATATGAAAACATGGCACATTGCATTTAT
GTGATGAGAAAACCAAAAGCGAAATTGAGTATATGAATATTGATATCGCACTATAATATAGAGGTTCG
ATAAACAGAGATAAAAATAAATTACACCCAATATATATAATTGTTGAGTAGATATTTATATATAATTCAC
ATCTAAACCGACCAACAGCCTGTAGCAAGCTAAATCTACCACTTGAAATACAAACAAAATATGCAGATG
AAATATAAACAAGCAGAGTTAATGAAATTGAATCAGAAACATAAGAATAAAACATGAAGATTATAAAA
ATGAAGAAAAAAACATATGTTTTAACTCCATCACTTCTTGATAATAATTCTGATGCAGAATAACTATTAT
CAGAAATATCTTTCACTAATTTTGTTCTAAATCCATAATCAGAGAAGTTAAGCACAAACCAATAAACAGT
AAACACATAACTAATAATACCAAATGTAGTAATATCTGACACATGTGTGAAATAAACAAAAACTCCTATT
TGAAATATTGCTTTAAGTAAAAATGTACCACCTGCATCAATCATTTTTCTAAACATACACTGACCATTTAG
AATAAATACATGGATTTTCACCCCTGCATTAATAACAGAGGTGAAATAGATTATTTATTCATGTTAATAT
AATTTAGCCTTTTAAAAATCTCACATACCACGGCATTGAAAGTCGTAATCCCTCTCTGATTTTTATATTTG
GTCTATACTTTAGTAAATCTATAGCCTTAGTAACATCAGCCTGAGAATGCCTAACATCTCCAGATCTAAA
CTCTCTATACTTAATGCTCAATTTATCGATATGGTGAATTAAATTAAGCTCATCATAAATGTAACCAGAT
AATTCATTTAACGTTGTTCTATCACCAACTGCAACATTATATATATTATCTTTAGCACTGTCCTTCGCTAAT
GCAGATAATATATTCATTTGTATAACATTATCTATATAACAAAAATCACGACTCGTTTCACCATCGCCATT
AATATATACGTCATCACCTTTAAGCATTGCTGCTGTCCATTTTGGAATTACTGCAGCATAAGCTCCATTA
GGATCTTGACGACGACCAAATACATTAAAATAACGTAATCCAATAGTTTTAAAACCATATGTTCGAGCA
TATACCTGAGCATAAATCTCGTTAACATATTTAGTAACTGCATAAGGAGAAAGTGGATTACCAATGTTTT
CCTCTACTTTTGGTAGTGCGGGATGATCTCCATAAGTTGAGCTTGATGCAGCATAAGTAAAACTTTGTAC
TTGTGCATTTTTAGCCGCATGTAAGATATTCAAAAATCCAGTAATATTAGTTGCATTGGTTGTTATAGGA
TCAACAATTGAACGAGGTACAGAACCTAGCGCAGCCTGATGTAAGACATGATCAACACCTTTCATAACT
TGCTCACAGGTAGTGAGATCTCGAATATCACCTTCTATAAAGCAAAATCGACTCCACTGTTCAGTGGAA
ACTAATGTTTTAACTTCATCAAGATTATATTGATGTCCCGTGGAAAAGTTATCTAACCCAATAACAACCT
GGTTTAATTTAAGTAACTTTTCTAAAAGATTTGATCCTATAAAGCCAGCGACACCAGTAATTAACCAAGT
TTTCGGTGAAAAAATTAACTGCTGTGTAATCTCTTCATAGCGAGACATATAAATATCCATGTATATTAAT
CAAGTGCTAAACCTAGCATAAAATGCTGAGGTGTATCATATCAAAGAGGATTTGATTTTTTTAATCACTC
TTACAAGCGCATATCGATACTTTTTTTATCAAGCACATATTTTAAATCGAACAAAACGTGCTCGTCTTTAC
CTAATGCACGAATAGCGCTCTCTCCCATCTCGCGAAACTCATTGTGTGCAACAGCGATAATTATTGCATC
ATAATGATTAACTTTAGGATCTTCACATAAAGTTAATCCATATTCATGTTGTGCCTCATCGGTAGAACAC
CATGGATCTATAATATCTATATTGATATTGTATTCTTTTAACTCTGAAATAATATCAATCACTTTAGTGTTT
CGTAGATCTGGGCAATTCTCTTTAAATGTAAGCCCCATCACTAACACATTCGCCCCTTCAACTTGAATCC
GTTGTTTCAACATTTTTTTGACTAACTGGGAAACGACATACTGCCCCATACTATCATTTAAACGACGTCC
GGCTAAAATCATCTCCGGATGATAGCCGACACTTTGCGCTTTATGTGTAAGATAATAAGGATCTACACC
TATACAGTGGCCACCTACTAAACCGGGCCTAAAAGGTAAAAAATTCCACTTCGTACCTGCAGCCTCAAG
AACCTCTAAGGTATCAATCCCTAACTTATTAAATATAATAGATAACTCATTAATCAATGCAATATTGACAT
CTCGCTGCGTGTTTTCAATTACTTTTGCAGCCTCTGCTACTTTTATCGATGATGCTTTATGCGTACCGACA
GTAATTATTAATTTATATAGCTGATCTACATACTCAGCAACATCCGGTGTAGATCCACTGGTCACCTTAA
GGATATTAGTTACACGATGCTCTTTATCCCCAGGATTAATACGCTCAGGTGAATAACCGGCAAAAAAAT
CAATATTAAACTTAAGACCTGATACTTTCTCTACAACTGGTATACAATCTTCTTCTGTCGCTCCAGGGTAA
ACTGTTGACTCATAAATAATAACATCGCCTTTCTTTATTATCTTACCCAATGTTTCAGATGCTTTAATTAG
AGGTGTTAGATCTGGCTGTTTATGTTTATCAATTGGAGTTGGAACAGTTACAATAAATACATTACACTCT
TTTAGTGCATCTAATGAACAGGCGTACGTCAATTTAGTTGCTTCTAACAACTCTAAATTTGAGCACTCTA
ATGTACTATCGTGACCATTTCGTAATTCATCAATACGAGACTTATTAATATCAAATCCAATCGTCGTTACT
TTCTTTCCAAACTCAACAGCAAGAGGCAATCCAACATAACCAAGGCCTATAATCCCAATTTTCGCATTCA
AAGTATCGAATTTCATTTTCTTATCCATCATAAAGTGCACATAGAACATCTATTTCGCATTGACACAACA
ATACGTAACCCAGTTTAATCTAACTTGGTTTTATCTTTTAATGTCGCATAAACTAATGTACCAACTATAGC
AAACATTGCCCCTGGTAATGCACCAAGAATCAAAATTAATGCTTTGTTGGGTTTGTCTTTTATAACTGGC
TCAGATGGCTTCATTTGATATCGATATGCTTGCGCCTGAATGTTATCGCGTAACATTCCTTCTAACTCATC
AATTTGACGTTTCACTTCGTAATATCTAAATGGGTAAATAATTGGCGCATCTTTAGTTGCTTTTAGCTCTG
CTGATAACAGTTTTTCGCCGAGCAAGAACTTATAGCTACTTTGCGCATTATCGAAGCCGCCTTTGCTAGA
GCGATAATCAATGATTCCGGCTTGTTCCGCAGTTGTTAAATCTCGCTGAATATGAGCTATTTGATCAAGC
TTTTCTGATTTTCTCTGGAAATCTATGTCAATAATTTCTTTATTTAGATTAACCAAAAGAGTATTAACATTT
TGACCAAACTCATCAAGACTTAATGTTACAGCTTGATTATTAACATTCTTTAGATATCCCTGCAGTAGTTT
ATATGCCTCTTGCGCTGAGTCTGCCGATGCTTGTATTATGTAGTAACTTGTATTTCCCTTTTCCTTTGGTG
GCGTAATCACAAGACCTTTATTCGCTTGCTCAGCTAACCAAATTTTCTTATCTTGTTCAGTAGATAAATTA
TTTGTTTCCTTTTTATAATACTCAGTACTCTCTAGAAATTTAACTTTGGTGTCTGGCGATTCAGCAATCAA
AATCAAATCGGAAAACAATCCATTAGCCAACGCATTCGTATCTAACGGTTGATTTAATATTCGGGCATA
CGCTTGGTGAAATTTAAGATAGCTGCCTAATTCAGCAATACGTGGTGCTTGAATATAGGTTTTAGCCGT
CCACTGCTCTTTTGCGGTAAAGGCATACACTGCTGCTAACCCCGTAGTCAGCAACGTGACACATAAAAT
AACCCATTTTTTCTTCCACAAGGTGCCTAAAAGCTCGAAAAGATCGATTTCATCCTCTGCTTTTGGCATAT
AAGTTGGATAGACCCCTTGCGGGATCAGATAAGGGGTCTGTTGTGGTTCAGATGCTTTTGACATGAAA
CCAATCCATTATTTATCAATGGGTTAAAAATAAAAATGAAGAATAAGCCTGACAAAAAAGTACGCTACC
GCCCTTGAGTTTCAGCTCCCAAAGCGCTGCGCAGAAACAGGATATAGCCAACTGCGATACAACAAGTG
ATATCTGGGATTAGAGGACATAATCGAGAACACTCCGTGTGAAGAGACATATACAGTCCCACACTTCCC
TGCTGCAAACCGGCGGATAGCCCGCAGAATTTGGAGTGCAGATTATAAAGCAATGCCTGTATAAGACC
AAATCTGCCTTCAAAATGATAACAGTATGTTTGTAAATTCTGTGAGGCTAAATAGCTGCGCTTAATACCG
CTACACTTTTGCCAGCCCATGTTTGCCTCCGGGGAATGGGCTGACGGTTTCCATAAAATGGCGAACTTT
TTTCAACAGTTGCCACATTGAGCTGCACTGATGATTACGCGTTATTGTGTCGTGAAGTGCCTGCCATAG
CCGTTCAACATGGTTCACCCATGGCGAGTAAACCGGCTGATAAATGACCCTGAACCACGGCAGCCTGG
CGGCTGCAAATCGGGGTGTGGCAGAATATGAGCTTTCAGAGTAACCATCCAGAATGAGTTCCCATGTC
ACACAACCTGGAGCACCAGAAAGTCCACACCAGAATGGTGAAAGAGGTTCTGAAAGCCGTCGCCAGA
GCCAATAATCACCCTTATCAGTCCGTATTTGCAGACTTTATCACCGGTCATCCCTCCTGCACGGTCTGTTT
CTGGGAAACCTTCCACAAAATGTACCCGGATTCACCGTACGAATACGTCACGTTCTGCCATACCTGCCG
TCGTTTTGATTTATACGAAACAGAAGCGGAGATGAAGGCGGATGACCCGAAGTGGTGGTGATTTTCAG
CCCCGACCGCTGAAACGCCTGTTCACAGCCAACCAGTGCTGGACGTCCTTCCTGGATGCGGGCGGTCTG
CGCGATATCGAGGTTGAGGCTGTCACAAAAATGCTGGCCTGCGGCACGCGGATACTGGGCGTGAAGG
AATACAACTGCGATAAACCTGAGTGCCCCCACGTCAGATACGTCACTAACTCATGCGGCAGCCGTGCCT
GCCCGTCCTGCGGAAAAAAGGCCACAGACCTGTGGATAGCGACACAGCTGAATCGTCTTCCTGACTGC
GACTGGGTACATCTGGTCTTCACCCTGCCGGACACGCTGTGGCCGGTGTTCGAAAGCAACCGGTGGCT
GCTGAATGACGTGTGCCGTCTGGCGGTGGAGAATCTGCTGTATGCCGCCCGGAAACGGGGGCAGGAA
CCCGGTATCTTCTGCGCCATCCACACGTATGGCCGTCGTCTCAACTGGCATCCGCATGTACATGTGTCTG
TAACCTGTGGATGTCTGAATAAGCATGGTCAGTGGAAAAAGCTGAGCTTCCTGAAAGACGCGATGCGT
TCGCGGTGGATGTGGAATATGCGGCAACGGCTTCTGAAAGCGTGGTCAGAGGGGCTGGCGATGCCGG
AGTCGTTGTCACATATCACGACGGAATCACAGTGGAGAAGCCTGGTGCTGAAAGCCGGAGGAAAATA
CTGGCATGTGTACATGTCGAAAAAAACGGCCGGCGGGAGGAATACGGCGCGCTACCTGGGTCGTTATC
TGAAGAAGCCGCCGATAGCGGCCTCCCGGCTGGCACATTACAACGGAGGGGCGAGCCTGAGCTTCCG
TTACCTGGACCACAAAACGGGAGAAACGGCGACGGAAACGCTGACACAGCGTGAGCTGGTCGCGAGG
CTGAAACAGCACATCCCGGAGAAGTTTTTTAAGATGGTGAGGTACTTCGGGTTCCTTGCCAACCGGGTG
TGTGGAGAGAAGCTGCCGCAGGTGTACCGTGCTCTGGGGATGGATAAACCGGAACCAGTGGCGAAAG
TGTGCTATGTACAAATGGTGAAACAGTTCCTGAGTCGTGACCCGTTCGAATGCGTGCTGTGTGGCGGC
CGGATGGTATACCGCCGGGCCATTGCGGGGCTGAATGTGGAGGGGCTGAAGAAAAACGCGCGGGAT
ATCAGTCTGCTGAGGTATATGCCGGCCTGACACAGGCTAAGTGTGCCCGGGAATCGGTGCAAGTATAA
AAAACAGCCTGTTGCGGTATAAGAATCGACCATGCAGAGAGAGAAAACACCGGAGTGGTGGGGAAAA
ACAGAAAGGCTCACGTGGCATAAGGCGCGGACAGGGGTATCGCTTAGTTTTTCAATTTCCTATACGTGA
ACTTCGGGTTCTCCTTCAGCCAGCTCTGTGTTTCCCGGCTTTTGTGGATAATGTAGTTGTCCACGATCAG
CGTGATGGTTTTCGCCCGACGGTATGTCGCTTTAAGCCGCTTCAGCAGGCTGATCGAGTAGGCAGCCTG
ACGGCTGCGGCTTGTCATGGTCTGCAATTACCGTTATAAAAACAGGCAATGTCATTGTCTTTCAGGTGG
TTATATGGCCCGTTCAGCTAAACCCCGTAAACGTAAACCCGCCTCTCAAAGAAGCAAACTTCCCCGCTAT
GTCGTGAAGCTTCACGAAGATGATTTTTTTGACGAAGAAGACGCAGAAGTTCTGCGCTTTGATAGTTTT
GACGATGCCGTTGAATGCTGCGCAGACCTGAATATTCCGTGAGGTAGCCTGAGTTTAACGGACACTCCT
TCCTGAAATAGAATGGCATCAGAAGGAGCTAATAATGAGCAGAAAAACCCAACGTTACTCTAAAGAGT
TCAAAGCCGAAGCTGTCAGAACGGTTCTTGAAAATCAACTTTCGATCAGTGAAGGCGCTTCCCGATTAT
CTCTTCCTGAAGGCACTTTAGGACAATGGGTTACCGCCGCCAGAAAAGGGCTCGGTACTCCTGGTTCCC
GCACGGTGGCTGAACTGGAATCTGAAATTCTGCAACTGCGTAAGGCGTTAAATGAAGCTCGCCTTGAG
CGAGATATATTAAAAAAAGCAACAGCGTATTTTGCACAGGAGTCGCTGAAAAATACGCGTTAATCGAA
CAATGGCGACAACAATTTCCCATTGAAGCGATGTGTCAGGTATTTGGTGTATCCAGGAGCGGTTATTAC
AACTGGGTACAGCATGAACCCTCAGACAGAAAACAAAGTGATGAGCGGCTAAAACTGGAGATTAAGG
TGGCACATATCCGCACTCGCGAAACATATGGAACCCGGCGGCTCCAGACGGAGCTGGCAGAGAATGG
CATCATCGTTGGTCGTGACCGACTGGCACGTCTTCGTAAGGAGCTAAGGCTACGCTGTAAGCAGAAAC
GCAAGTTCAGAGCGACTACGAACTCGAACCACAATCTGCCAGTTGCGCCAAATCTGCTGAACCAGACG
TTCACTCCTACAGCACCAAATCAGGTCTGGGTGGCGGACCTGACGTATGTTGCCACACAGGAGGGATG
GTTGTACCTCGCTGGCATCAAAGATGTTTATACGTGCGAAATTGTCGGCTACGCCATGGGAGAGCGCAT
GACAAAAGAGCTGACAGGTAAAGCCCTGTTTATGGCGCTCAGGAGCCAGCGCCCACCTGCCGGGCTAA
TCCACCACTCTGATCGAGGTTCACAGTACTGCGCATACGATTACCGGGTCATACAGGAGCAGTCTGGTC
TGAAAACATCAATGTCGCGTAAAGGTAACTGTTACGACAACGCTCCGATGGAAAGCTTCTGGGGAACG
CTGAAAAATGAGAGCCTGAGCCACTATCGTTTTAATAACCGGGATGAAGCCATCTCAGTAATACGGGA
ATACATTGAGATTTTCTACAATCGTCAGCGTCGTCACTCTCGTCTGGGGAATATCTCCCCGGCAGCCTTC
AGGGAAAAATATCATCAGATGGCTGCTTAAAAAAAGAACAAATGGTAGTGTCCGCTATTGCCAGTACA
CCTCACCGTTCTWTRTGGATGCGGGAAACAAAAAGCTGGTCTTCTGGTTTGTACGTGTTGATGACGAA
GGGTATCCGGAAATCGCCCGCTGCACGGAGCGGGAATTTGCGACCATTCTTTCCGGTATCAGCGCCGG
CGGTATGTACTGCCCGGAGTGTGGCACGGTTCACTGGCCGGACGGAGTCGCCCCGCCCTTCTGATGCT
TCCCCGTTTTGCCGATATCTTTCAGCAGGGAAACCGCTGGCTTAACTGGCTGGAGAAGCAGCCGGAAG
GGGCAGTACGTCCGGTGGTGATTGAGTCGGTGACCAAAATCATGGCGTGCGGGACCACACTGATGGG
GTATACACAGTGGTGCTGTTCATCACCGGACTGTTGCCATACAAAAAAGGTCTGCTTCCGGTGTAAAAG
CCGCTCCTGCCCGCACTGCGGGGTGAAGGCTGGCGCACAGTGGATACAGTATCTGCTGAGCCTGGTCC
CCGACTGCCCGTGGCAGCATATTGTGTTCACACTTCCCTGCCAGTACTGGTCCCTGGTGTTCCACAACCG
GTGGTTACTGACAGAGATGAGCCGCATTGCAGCGGATGTGATACTGGAAATCTGCCATCARRMMRMY
RWRRMRWMRRRRMWMWWMACGGTGATCCACACATGGGGGCGTGACCAGCAGTGGCATCCGCA
TATCCATTTATCGACAACTGCCGGTGGTGTGACGTCGGGCCACACCTGGAAAAATCTTCATTTTTACGCC
CGTAAGGTGATGAGCATGTGGCGCTACCGGATAACACGGTTGCTGTCACGGAAATACCCTGACCTGGT
GATGCCGGATGCGCTGGCAGCAGAAGGAAGCAGTAAACGGGAATGGAATCGCTTCCTGGACACGCAT
TACCGCCGTGGCTGGAATGTCAACGTATCCCGGGTGATGGATAATGCCACCCATGTGGCGGTGTACTTT
GGCTCTTACCTGAAAAAGCCACCAGTGCCGATGAGTCGTCTGGAGCATTATGCCGGTCAGGATGAAAT
CGGTCTGCGTTACAACAGTCACCGAACAAAACGGGAAGAATACCTGTTGATGAGTGGAGATGAGTTCA
TGGAAAGGTTCTCCTGGCATGTGGCGGATAAGGGGTTCCGTATGGTGAGGTACTACGGTTTTTTGAGT
CCGGCGAAACGGCGGTTACTGGAAGAAGTGGTGTACATCATAACGGAGACAGTGAGAAAAACGGCGA
TGCAAATCACCTGGAGAGGGATGTATCAGAGGTTACTGAAGGTTGACCCGCTGAAGTGCGTGCTGTGC
GGGAGTCAGATGCGTTTTACGGGGCTGAAGCGGGGCTACCGTCTGGCAGAGCAGGTCCTGATGCATG
AGCTGCTGGCCCGGATGCGGTGGTGCGGCTGAGAGCCGCAGAGGGGAAGTTGCGTCCATTTTGAGGG
AAACGGAGCAAAAAACCGCCATTCATACCCTGAATCAATCAGAGCCATCCAGTTTAATCGTCGGTTCCG
TTCATATGGAGCAAAAGTGGTGTTGAAGAAACATCCGTTTTGTGGTGTTTTTTTAATCTTTTTGGGGTTT
TAATTCCTATCGATCAAGCGGCTGGCACGTAAAACAATCTGCTTCTCGCGTTCCGTGGAAATCCACGAA
AAGTCATCGGCTCCTTCATTGAAAAACACATATTCTACTGATTGGAAGCGCCACCCGAAAAAGTATCGT
TTTTTTACAGTAAACTTCATTTAGCCGACTATAATGTAAAAAAATAAATTTTTTACCAAAATATAACATAA
GGAAGTAATATGCTTACACAAACTATATTCCCTTGTCTTCCCAAAAAATAAGAAAATATCATCCCTGAAG
TATCAAATACTATATTATTATCTTCTACTGTTACTACAGATGGATATACTGTTTTTAATAAAAAAAACAGC
CATATACGAATTGCAAATACCTGCTACCAACCGAACGAAAAGGTTGAAATTCACAGCTACAGAAATACA
ATGGTTGACAAAGATAAATGAAGCCGGAATTGATAAAAAACAAAGCCAACGGTATTCTGATTTCTGAA
GTTAATTTCAGACTCGTTTAAGCTCTATTTTCAAAAAAATATATCAGGGTTTGTTACTAATAACTTCTGAA
AACAGAAAAAAATAGAGCAGTAATTCTATTTCAGAGAGGAGTAACTCCATCTATCCTCGATACGTACTG
AACGTTATCGTGATATATTCATCATGCTTTCTTCACCATTATTCGAGTACAGGGAGAGACGCAATGACCT
TAAATACCTTTTTATGGCTGAATTACCATTCTCAGGAATAACAGCAGACTCCTTCCCATAGACGGCTTAG
GGGGAAAGCAATACCCGGAGAAAGGGTACAAAGCAAACTGTTTTAAAATTAATAAGCTATATTCTATTT
CACTAGAATTCTTATCATTGATAAGAAATTTTAGTTATAATGATTAAATCAACCAATATACAGGCAATCG
GTTCTAGTATTATGCATCAAATAAACAATATATACTCGTTAACTCCATTTTCTTTACCTATGGAACTGACT
CCATCTTGTAATGAATTTTATTTAAAAGCCTGGAGTGAATGGGAAAAGAACGGTACCCCAGGCGAGCA
ACGCAATATCGCCTTCAATAGGCTGAAAATATGTTTACAAAATCAAGAGGCAGAATTAAATTTATCTGA
GTTAGATTTAAAAACATTACCAGATTTACCGCCTCAGATAACAACACTGGAAATAAGAAAAAACCTATT
AACACATCTCCCTGATTTACCACCAATGCTTAAGGTAATACATGCTCAATTTAATCAACTGGAAAGCTTA
CCTGCCTTACCCGAGACGTTAGAAGAGCTTAATGCGGGTGATAACAAGATAAAAGAATTACCATTTCTT
CCTGAAAATCTAACTCATTTACGGGTTCATAATAACCGATTGCATATTCTGCCACTATTGCCACCGGAAC
TAAAATTACTGGTAGTTTCTGGAAACAGATTAGACAGCATTCCCCCCTTTCCAGATAAGCTTGAAGGGC
TGGCTCTGGCTAATAATTTTATAGAACAACTACCGGAATTACCTTTTAGTATGAACAGGGCTGTGCTAAT
GAATAATAATCTGACAACACTTCCGGAAAGTGTCCTGAGATTAGCTCAGAATGCCTTCGTAAATGTTGC
AGGTAATCCATTGTCTGGCCATACCATGCGTACACTACAACAAATAACCACCGGACCAGATTATTCTGG
TCCTCGAATATTTTTCTCTATGGGAAATTCTGCCACAATTTCCGCTCCAGAACACTCCCTGGCTGATGCC
GTGACAGCATGGTTCCCGGAAAACAAACAATCTGATGTATCACAGATATGGCATGCTTTTGAACATGAA
GAGCATGCCAACACCTTTTCCGCGTTCCTTGACCGCCTTTCCGATACCGTCTCTGCACGCAATACCTCCG
GATTCCGTGAACAGGTCGCTGCATGGCTGGAAAAACTCAGTGCCTCTGCGGAGCTTCGACAGCAGTCT
TTCGCTGTTGCTGCTGATGCCACTGAGAGCTGTGAGGACCGTGTCGCGCTCACATGGAACAATCTCCGG
AAAACCCTCCTGGTCCATCAGGCATCAGAAGGCCTTTTCGATAATGATACCGGCGCTCTGCTCTCCCTG
GGCAGGGAAATGTTCCGCCTCGAAATTCTGGAGGACATTGCCCGGGATAAAGTCAGAACTCTCCATTTT
GTGGATGAGATAGAAGTCTACCTGGCCTTCCAGACCATGCTCGCAGAGAAACTTCAGCTCTCCACTGCC
GTGAAGGAAATGCGTTTCTATGGCGTGTCGGGAGTGACAGCAAATGACCTCCGCACTGCCGAAGCCAT
GGTCAGAAGCCGTGAAGAGAATGAATTTACGGACTGGTTCTCCCTCTGGGGACCATGGCATGCTGTAC
TGAAGCGTACGGAAGCTGACCGCTGGGCGCAGGCAGAAGAGCAGAAGTATGAGATGCTGGAGAATG
AGTACTCTCAGAGGGTGGCTGACCGGCTGAAAGCATCAGGTCTGAGCGGTGATGCGGATGCGGAGAG
GGAAGCCGGTGCACAGGTGATGCGTGAGACTGAACAGCAGATTTACCGTCAGCTGACTGACGAGGTA
CTGGCCCTGCGATTGTCTGAAAACGGCTCAAATCATATCGCATAACCCACTGATMMTGTTTACTACCGG
ATGTAGCTTTTTTCATGCCCGAGATGGTCAGTTAGTTCCGCATTAAGCACCGTTTCGACAGTTAATTTCG
TCTGCATACGGGAAAACTGATTGAGGTCGGCCTCGGTTTTGAGGCCTTTAGCCAGTTCAGCCGCAAGG
GCTTTAAGCTTCTTTTCGTCCATAATTTACCTGTCTCCGTTGTTGGAGTGAACATATCAAAAACAGGCAA
TTACACAATTTAAATTACAGTCTCAAAACAAAACCAACTGTCAATAATAGTTTACCAGCAAGTTTACTCG
CAGAGTGCCAGGCTGTGTCCCTTAATTACGTAAGCGTTGATAAAAGAGTCGGATGCAGCCCAGTTTCAC
CATCGCCAGGTAATTTCTCGCCGTTTTGTCGTAACGCGTGGCGATGCGGCGATATTCTTTCAGCCGCCC
AAAGCACCGTTCAACGACATTACGATTGCAGTACGCATCACGATGAACCACGGCAGCCTGACGGCTGC
AAATTGAAAACCGCCCCTGTTCGTCCTGTTGTTGCCGCACGCTCCGCAGAATATGTATGCTCTGAGGAC
CTGTTTTATTAAGGACTCATCCTCTTATGTTGACCCGCAAAAGTATAGATACCGTTCTGCTCTCTGTTGGT
GCTGAGAAGCTCTCACAACGTGAATGGGACTGGATGAAGATGCTCAAACCCATGGACCCACCACCTGC
GATGGTCGCGGCCTCCATCCTGGAGCGCCGTGGCGACACAGCAGCCCTTACCCGGCTACAGGATACCG
GGGGTTAATCCGTCCTCATGTTGTCCGCTTTTACTCCCCGCCCGCTGAAACGCCTGTTCACAGCCAACCA
GTGCTGGACGTCCTTCCTGGATGCGGGCGGTCTGCGCGATATCGAGGTTGAGGCTGTCACAAAAATGC
TGGCCTGCGGCACGCGGATACTGGGAGTGAAGGAATACAACTGCGATAAACCTGAGTGCCCCCACGTC
AGATACGTCACTAACTCATGCGGCAGCCGTGCCTGCCCGTCCTGCGGAAAAAAGGCCACAGACCTGTG
GATAGCGACACAGCTGAATCGTCTTCCTGACTGCGACTGGGTACATCTGGTCTTCACCCTGCCGGACAC
GCTGTGGCCGGTGTTCGAAAGCAACCGGTGGCTGCTGAATGACGTGTGCCGTCTGGCGGTGGAGAAT
CTGCTGTATGCCGCCCGGAAACGGGGGCAGGAACCCGGTATCTTCTGCGCCATCCACACGTATGGCCG
TCGTCTCAACTGGCATCCGCATGTACATGTGTCTGTAACCTGTGGATGTCTGAATAAGCATGGTCAGTG
GAAAAAGCTGAGCTTCCTGAAAGACGCGATGCGTTCGCGGTGGATGTGGAATATGCGGCAACGGCTTC
TGAAAGCGTGGTCAGAGGGGCTGGCGATGCCGGAGTCGTTGTCACATATCACGACGGAATCACAGTG
GAGAAGCCTGGTGCTGAAAGCCGGAGGAAAATACTGGCATGTGTACATGTCGAAAAAAACGGCCGGC
GGGAGGAATACGGCGCGCTACCTGGGTCGTTATCTGAAGAAGCCGCCGATAGCGGCCTCCCGGCTGG
CACATTACAACGGAGGGGCGAGCCTGAGCTTCCGTTACCTGGACCACAAAACGGGAGAAACGGCGAC
GGAAACGCTGACACAGCGTGAGCTGGTCGCGAGGCTGAAACAGCACATCCCGGAGAAGTTTTTTAAG
ATGGTGAGGTACTTCGGGTTCCTTGCCAACCGGGTGTGTGGAGAGAAGCTGCCGCAGGTGTACCGTGC
TCTGGGGATGGATAAACCGGAACCAGTGGCGAAAGTGTGCTATGCACAAATGGTGAAACAGTTCCTGA
GTCGTGACCCGTTCGAATGCGTGCTGTGTGGCGGCCGGATGGTATACCGCCGGGCCATTGCGGGGCTG
AATGTGGAGGGGCTGAAGAAAAACGCGCGGGATATCAGTCTGCTGAGGTATATGCCGGCCTGACACA
GGCTAAGTGTGCCCGGGAATCGGTGCAAGTATAAAAAACAGCCTGTTGCGGTATAAGAATCGACCATG
CAGAGAGAGAAAACACCGGAGTGGTGGGGAAAAACAGAAAGGCTCACGTGGCATAAGGCGCGGACA
GGGGTATCGCTTAGTTTTTCAATTTCCTATACGTCGAGTCCACAGAGAAAATAACAATTCTGAGTTATCT
GCGGGAAAGGTTATTTATATGTTACTATCTCATTCTCTCACTGGCGAGATTATAAACGTTACTACGTTCT
CGTTTTCCTACCGCAACAACCGCAATAATTAACATATCGTCAATCACCTGATAAACCAGACGAAATCCCG
ATGCACGTAGTTTAATTTTGTAGCAGTCCTTTAGCCCTCTTAGTTTTGCCGACGGTATATGAGGATTTTC
GCTGCATTTTTTTAGTTTTTTCACAAACTGTTGTTGAATGGTCTTATCCAGTTTCAGCCATTCCTTGAGCG
CATCATCCCTGAATTTTACCGTATAAGTCATAAATAACTGTCCAGGTCCACATCATGCAGTGGCTGATTG
CTGCGTTCAGTAACCAGTTTCACCAGTTCCTGGTCATCCAGAGCATCAAGCATCCTTTCATACAGTTCAG
CAGGCACACAGTAGAATGCCGGCTGATTCCGGTTAAGAATGGCGACAGGGTAACCATCTCCGGCACTG
ACTGTAGCCATAGGATTTTTTTTCAGTTCACTGACACTTGCACTGGTCTCAGACAGAATGATGTTAGGCA
TAACGCACCTTTGAGACTGTTTAACAAGTCTTAACGATATCCCTGAAACGACCACTTAACAAGTCTTTAT
TACTCACACTTTTGCAAATGCATTAAATGCTGCTTGTATTATTCTGATGCCGCGCCAGCGGCATGTAAGG
CGGCACGCTGCGGTTACATGTGATACCGGAGTAAAACCGCCGAAGCCCGGCGTWAGCCGGTACTGAT
TGAYAGATTTCACCTTACCCATCCCCAGCCCTGCTAAGCCATACCCGTTTTCAGCCATGACAGAGCTTCT
GTGCGCGGTCGGAGTGGTCCCGACGAGGGTTTACCCGAAGTCGGGGCGTATCTCCGCGTTAGCGGGC
CGTSAGGGCCGCTTACGAGCGTGTACTCRATACKTCCAGCSAGAAGACTGACAGCGATGACGRWGTAG
TTACAACATTCATAATTAAAAGCGAYKYTGTTCCCGCCCTTYGGGCGGGGGCRGCSKCGCTTTTCAGTTA
TGAGGAGGGGCGTTGTGGTTTCAGTTCTGCGTCGGACTTGGGTTTTTTAGGGGTTTATGTTTGCATGTT
GTAACTAAATCGGCTTCGGTCGGGGCCCGCCGCTTGCGGTGGGAGGTGCATATCTGTCTGTCCACAGG
ACAGGCAGTGAACAGGTTTTCTTTTTAAATGGATGTAATTAAGTAGTTTAAAGGAGATATAAACAGCTG
TTTAAAAGATACATTGCACCCTGTAAAGCTAACGGCTGGCGCTTTATGACATGAACGGTTGTAACCTTA
TGGGGAAGTCCCTTGCAGTTTAATGTGGATAAGCAAAATTCCCCGTCTGTGAGGCGTGTTTTGTATCAA
AAACAGGGGGGACCGGATGCACCAGAAGGTGGATGATGAGGTTGTTTTTTGTATGTAGCGCTGATTTT
TTGTGCACTGGCGGGCTTCAGGCGTGCGAATGCCTCCGGCGCGTGCCGGATTATTCTGAGGAGGTCAC
TTTCAGGGGGAAGCTGTGGCCAGCCGGCTGTAATTACGGTTACGTGACAGAATCATGCGCTCCTTCACA
CGACGCTCCACTTCACGTTTTACCGCCTCACGACTGGCAGTGAAGCGCCCTTCCGAGATTTCACGCGTCA
GTTGCCGTTTCACCAGGGTGACAATATCCTGACGCTGCCTGTCCGCATCACGACGCGCACGGGCACGCT
TCATTCCACGGGACTTAAGCTCTGTCTGGTAACTGCGGAAACGCTCACGCACAAAACGCCAGGCTTTCG
CCATCAGTTCATCCATACCCAGGGTATCCAGTCCCTGCTTCTTGCGCTGTCTGTTTTCCCATTCCACACGA
CTGCGGCGCGCGGACGCCACCGCCTCCTCTGATACATCGAGGGCGGCAAACAGCGCCGGTGTGAACGT
GATGTCGGTCGGAATGTTGCAGCCGATAAGCGGGTCATATTCCGTCTGGTAGGTAATCAGCCCCAGCT
CTGCCAGGAACGTCAGGGCACGCGTGGCACGGGTGATGGAAAGTGTTCCGGCATCAGACTCCGTCGC
CAGTCCGCACTCAATGGCCAGCGTGGTGATGGAGCACTGAACGCGGTTGGCCAGCGGGTCATAGTGG
AAACACAGCCCCTGCAGCAGCGCATCAATGGCCCGGCGACGTAGTACCGGTGGCATGCGCCGACGCA
GACCAAGCGAACGGGCATGTGCCACATGAATGGCGAAATCAAAACGGGAGGTGAAGCCCACCGCCTT
TTCCATCAGTTTCCCGCAGAACGGAAGCGTCCGGGCACCTTCACGGGGTGTGAACACCGGATTCGGGT
TCTTTACCTGGCGGTAATAAGTTTGTTGAAGATCAGTCACACCATCCTGCACTTACAATGCGCAGAAGG
AGCGAGCACAGAAAGAAGTCTTGAACTTTTCCGGGCATATAACTATACTCCCCGCATAGCTTGATTGTT
GGCTATACGGTTTAAGTGGGCCCCGGTAATCTTTCTGACTCGCCAAAGTTAAAGAAGATTATCGGGGTT
TTTGCTTTTCTGGCTCCTGTACATCCACATCAGAACCAGTTCCCTGCCACCTTATGGCGTGGCCAGCCAT
AAAATTCCTTAAACGATCAGTAATCTAGCATGCTACGCCACAAAGTAAAGTCTTTACTTTAGTATATCCA
GTCTCTGCAGTTCATCTTTGATGATTTTCTCAACGAACTGAGCCTGTGTTATCCCCTCTCTCTCGCAGTAC
TCAACCATGAGATCTTTCAGAGGATTTTTGACAAAAACTTTTATCTCTTTGTGTGTAAGACGTTTTCTTGC
AACTGCGGCCATTTGTTTCTCAGAGTCAGTCATAGGCTTACCTCTGCGCACAAACCGCTTTTGACTCAAT
GAGGAAGTCACTGCATTTTCTGTCTGCGACATCTCGCCTCCTCAATACTCAAACAGGGATCGTTTCGCAG
AGGATACTACAGTTTTTTGAAATCAGCGACTTGAGAATTGTGGCGAAGATCTGTGTTGGCGCACAAATC
AACGGGGATTACTGTCGTTTAATGTGATTTAAACTGTGAAATAGTATGGTTTTCAGTTATGGAAACGCC
GGGAGCGAGGAAAACTTGCTTTTTCCCGTTTCCGGGGTTGGACAACTAAGCAACGCGAAGGCGTCAGC
TACGATGTTCCGGTGACTGCTGATCAGCCCCTGTTGTATTCAGCCATTCTCCGGTCATCTGCAGCAGTTT
TCTGGCATCGTGGCTTCTGAGCAACGGAATACCAGCCTGCCTCAGAACCTCTTCCAGAAGAATATCCCG
GCGTCTGCGTTCCGGTCGTAAATGGCTGGCATCATCCAGCTCWAMWRMWRMMMMMMWMMWR
MMMRMMMRMMRMWMMMMRMWMMMMMMMWMMMYMWRMMMMWRMRMMMM
MMKMMWRMMWMMMWRMMMMMMMWRKMRWRMWMRWRRMMRRMWMWWMMMR
WKMMKMWRRMMRMWMWMMRMRMMMMRWMMMWRMRSMMMMMRMWMMMMMM
RYTTCGGTGATGCTGCCAACTTACTGATTTAGTGTATGATGGTGTTTTTGAGGTGCTCCAGTGGCTTCTG
TTTCTATCAGCTGTCCCTCCTGTTCAGCTACTGACGGGGTGGTGCGTAACGGCAAAAGCACTGCCGGAC
ATCAGCGCTATCTCTGCTCTCACTGCCGTAAAACATGGCAACTGCAGTTCACTTACACCGCTTCYMAACC
CGGTACGCACCAGAAAATCATTGATATGGCCATGAATGGCGTTGGATGCCGGGCAACTGCACGCATTA
TGGGCGTTGGCCTCAACACGATTTTACGTCACTTAAAAAACTCAGGCCGCAGTCGGTAACCTCGCGCAT
ACAGCCGGGCAGTGACGTCATCGTCTGCGCGGAAATGGACGAACAGTGGGGCTATGTCGGGGCTAAA
TCGCGCCAGCGCTGGCTGTITTACGCGTATGACAGTCTCCGGAAGACGGTTGTTGCGCACGTATTCGGT
GAACGCACTATGGCGACGCTGGGGCGTCTTATGAGCCTGCTGTCACCCTTTGACGTGGTGATATGGAT
GACGGATGGCTGGCCGCTGTATGAATCCCGCCTGAAGGGAAAGCTGCACGTAATCAGCAAGCGATATA
CGCAGCGAATTGAGCGGCATAACCTGAATCTGAGGCAGCACCTGGCACGGCTGGGACGGAAGTCGCT
GTCGTTCTCAAAATCGGTGGAGCTGCATGACAAAGTCATCGGGCATTATCTGAACATAAAACACTATCA
ATAAGTTGGAGYMMWWRMMMMYMKMWWMMWMWMMMMMMWWMWRMMMMRWMS
WWYMMWMMMMMWRRMWWMMMWWWMWKWMRRMMRMRWMWWMWRMMRWWR
MYMRWMWWMAMMRMWRRMMMRWMWWMRMWMWRMRWMAMMRRTMMGWATACMT
GCCGCATGCAATTGCATCAGCAAAGGAGAATGCTGATTTACACTTCTGAGGATAATCCGGATAAGTAG
CGCTGCAATAATCAGAACAATGAGAATAAAAATAATCAACTTCATTATTTATCTGCTCCTGGCAATTAAT
TATTGCTGAATTATTACCTTACATATTTCCATATCTCTTTTGGTATCCTGTCATATAACTTTCCGGTGATTA
ATTCCGCTAACCGGTGGTCTGCTGCTGAATTAAATGATTCACGTTCACTGTTTGATAAACTGTCTCTGTT
TTTATGAATAATTTTTTCCAGCGTATTGACTGATGAACATCGCCGTAACTGATACAGCCATTCCTGTTTTG
TTTTTGCCATTTTTATTCCCGCTGAGAACAGATGAATGATAGGATTGCCATCGCTTTATTTACGGTGCAT
CCATATCCAGGGGGGAACCGGATCAGCATCTGACCAGAGCCCACGTTTTTGTTGTCTGGCTTCGTTCTG
AAGGACAGGTAGCACGGGATCTGTGTTGTACTGCTCATATACCCAGGCAGCCCCGGCCCGGACCATAA
ACTGATTAATATTCATTCCGTCTGGTGCATAAACCTGCCCGAGAATACGACCATAACGATCTCGCTGAA
AATATGTGACGGTAACCGTTTTTCCTGCGACCAGTGATTTCATCATGTCTGTTGACCACCGGCCATAATC
CTGTTTCTTTTCCGGTGCATCAATATTTACCAGCCTGATCCGCACCGCCTTCAGTGAGTCCATGACCTCG
ATGGTATCACCGTCCAGTACACGAACAACCCGTCCATGAATATCTGCAGACAATACCGGCCATGAAAAG
ATAAATAATACCAGTGGAATATATTTCCTCAWGTTMWWMTMSMWMWMMRMWMMMWRRMM
YMYRWRWWWMMRSCMYCCTGAATAKKGCAGCMCACAYTGAATMACAMCTTATTTTGTGAAATTA
ATCACAAAATATAAAAWMWCGACTTAATRCGGAAAARACTCTGTGCTACTCAGAACTGTCCGGTCATA
CTGCCCGACCGGATATTAARAAGCAAATCTMATAAACTCCTTTCAGCCTTCTGACTCCTGGAGTAATGA
AGGCTGCTTTTTTCGTTACACCCTTCCCCGGCAGCGCCGGTGGMAGTGTTMAGCCCTWWWTCGGGC
AAACTGCTGCCAGTCTGTMMTRRRMRMMMMMMARGCARMMCGTMGSMAWGMTTMTGMMG
MTTMMATMMMGMRWMMMMWRAMWTMMMRMMTMAMRGMMCGMMMGMMRRMAMR
MKRRTMMMGMMMRAMMWRRMMRWWMMRWRMMMWGKWWMWRMMYMMMYWMMW
WWWMWWGTCMCCCTTTAGTTGTCAGTATTATTGTGATAAATAATTCMGTGGACTGGCTWMMTW
WCGGAAGCGRAMRTWAWWWCMWWWWAMRMMWWAWWWMMMWRMMWMMTMTMW
GATGGARGCTWTWTWWTATTTCTCATCAAGCACGGCCTGAAGTTCTGCCTTTATCCGGTTCTGGCGG
CGGATTTTATCCAGACGCGCTGCCGCATACGCTTCYTCCTCCTGAGAAATATGCTCCGTCACATACCCTT
CCGTGTCATACCGGCAGGCACCGGCTTTCATGGCACACAGATAGCTTTCTGAACGGGTGATGGCMTTC
AGCGCCCTGCGCAGTWWTTTATGCGAGAGCGGGATAWTCMGCTRMGCCACGTCTTCCAGTAACACG
TCCCGGATACCGCAGGCCAGAAGCCGGGGGGTGTCACCGTCAAATAATCCCGGCCACCAGGGTTTCAG
GGTAWTCAMGGCCTCATCMAGMGTGGGCAGGTTMARMWMMMWRRMMMRTRMMWKWMWR
GMMTGCGCWWWWWWWKMMRMMMRMWMWRMWWMMMRRRMWRMWWWMWMGGM
MMRCWWSWGCTWWWTCACCTTCCACTTTGGTGGCGTGGTGAMMWWRMWRATGRTTTTCCRGC
TGCRRAMARRMRTMTMMMMWTMMRWWWKMMRMWWMMRWRWMMRWMMMRRWMR
MWWSWRMKMWRWMMWRMWRWRWWMWRMMWMWRMMRMMWMMWMMWRWWM
MRRWMMMMRMWAMMMYAMMSRWMMMAGMGCMAGAACGCCCAGCACAGCCAGATGACAG
GCGTAGAATACCGGGAAAAAATCCCCGGGCCAGAAACGGGGTAATGATTTTCCTGCACACGACACCAG
ACCAACGGTCAGCACCGTCATCACAAGACCGGCCACCGCCGCTGCATCACTGGTGGCAAGGTTAAGCG
CCGGAATAACAGCCAGCAGGCAGGCGACCAGCGCCAGACGTTCCGCTCTGTCTTCCGCCCGGTACAGT
CGGTGGCTGACTGCCAGCATCAGCAGCCCGGCAATGCCGTAACTGGTGCCGGACAAAGGCCCCCAACA
GGGCCATCAGAAGAATGGCCGCTGCTGTACGCCACMMACTGCGCGTWTMACAMMACGTCAGCAM
MTGTRSMRMMMMWRMMMMRRMMMMMMRRMWMWWMMMMWMMWMMMMRRRMM
MRMMWRMMWRMWMMWMWRMRMMMWRRRMMMWMMWWMMMMMTMMMMMMM
RMMRRWWMMWRRMMRRWWSMMKRMWMWRCGMMWRMMRRRRCMRMWWMMRMMM
MMACACCAGGGCMAMMAGCRRMMMRRMWMMWMRMMMRRMMMRMMMMMWMMMW
WMMWRMWWMMRMWRRMMWRWMMRMWWRMWRWRRWMMMRWMMMMWMMRMM
MMMRWRMMMMMRWWWWRMWRMWMWMMMRMWRTMMRRRTRMMMMRMMMMRM
MMWRMMWRMMMMMMRRYMWMRRWMMRKRMMRMMRRWRMMWMRWWMMRWRWM
RWRWWMRWRWKMWMMRKWRWMMWMMRWRWWWRWMWWMMWRWMWMMRYMW
MMRMMMRRRRWWWWMWMWWTMTGMMRRTCAMGMAMMMWMTMMMGMTMMATCTG
CTGCAGAMGCTCCTGCAGCAGATTTTCMCGGGCAACCTCCCGGACCGCTTCCCGCTCCCGTTGTGATT
CACGCAGCACACTCTCCGGCAGCGCGGCTTCCCGTTCAGTTATGGCAGCCCGGTCACGCTCCTGCCCGG
CAATTTCCMTGACAGCCAGCTCTGWWTTGCCGTCCGGCARRTMMRRWWWRWWMYMWRMMAW
WWWAMRRMMMMWWWMMWMTRMRMRMMRWWMMRWWWMMMRRMSRMWRRMMW
MWWMARMMTRMMRMWRWRMMMRWAMMTMWRMMRWRAMMRGTWCGCCATTTCMGGC
TCCCGGCTGGACGCTGTTGTCCGGGATATCCCCCMACAMGCRGCCATCGGTGATRGCACMRRRAWW
MMMMRRAMRRMMWWMMMMRRMMRWRMWWMMYMMMMMMMMMMWRWMMRRM
WWMWMWMRRRMMMWMMRRMMRMMMWMMWRMMWMWWMWMMRMMMRMMRR
MWWWMWMMRWWMMRRMWRMMMTRMARRRMMAMRAMMWRMRCMWMMMMGMTG
CMTWWWACMCGACMTTCACCGCTGAATCCMMGMAGCCCGWWTCMGTCATCCGTGGTCAGCGR
RTTCARCCASAWAMMRRMRRMMWWRMMRWWMMRRWMMMMMRMMRRMMRWRSMMM
MWMMRRWWRMRSMWMWYWMMRWMMMRKMRMMMWMMMMMRWRMRRRACTRTMT
MMMMCGGCCAGCMCCGCCTGACGGAGAACGGCACGGMMTGMMRMMAMRTMMMRMARMT
MMMGMGMMGTAYTRMAMMSMMGMWMWRMMWWMMWRMMMWMMMRRWMMRRW
WWMRRMWMMMMMMMMWMRWRRRMMRWWMMWWWYKRWMMRRMMWWRWWRM
WRRMMWMMRWMMMRMMMWRMMRRYWMWMRRWRWMMMMMWRMWMMWRMWR
MWWMMWRMRMKMMMRRRMMMMRWMSRMWRMMWMMMMRMMRRSMMTCATGAGGT
GTACTGGCAATAGCGGACACTACCATTTGTTCTTTTTTTAAGCAGCCATCTGATGATATTTTTCCCTGAA
GGCTGCCGGGGAGATATTCCCCAGACGAGAGTGACGACGCTGACGATTGTAGAAAATCTCAATGTATT
CCCGTATTACTGAGATGGCTTCATCCCGGTTATTAAAACGATAGTGGCTCAGGCTCTCATTTTTCAGCGT
TCCCCAGAAGCTTTCCATCGGAGCGTTGTCGTAACAGTTACCTTTACGCGACATTGATGTTTTCAGACCA
GACTGCTCCTGTATGACCCGGTAATCGTATGCGCAGTACTGTGAACCTCGATCAGAGTGGTGGATTAGC
CCGGCAGGTGGGCGCTGGCTCCTGAGCGCCATAAACAGGGCTTTACCTGTCAGCTCTTTTGTCATGCGC
TCTCCCATGGCGTAGCCGACAATTTCGCACGTATAAACATCTTTGATGCCAGCGAGGTACAACCATCCCT
CCTGTGTGGCAACATACGTCAGGTCCGCCACCCAGACCTGATTTGGTGCTGTAGGAGCGAACGTCTGG
TTCAGCAGATTTGGCGCAACTGGCAGATTGTGGTTCGAGTTCGTAGTCGCTCTGAACTTGCGTTTCTGC
TTACAGCGTAGCCTTAGCTCCTTACGAAGACGTGCCAGTCGGTCACGACCAACGATGATGCCATTCTCT
GCCAGCTCCGTCTGGAGCCGCCGGGTTCCATATGTTTCGCGAGTGCGGATATGTGCCACCTTAATCTCC
AGTTTTAGCCGCTCATCACTTTGTTTTCTGTCTGAGGGTTCATGCTGTACCCAGTTGTAATAACCGCTCCT
GGATACACCAAATACCTGACACATCGCTTCAATGGGAAATTGTTGTCGCCATTGTTCGATTAACGCGTA
TTTTTCAGCGACTCCTGTGCAAAATACGCTGTTGCTTTTTTTAATATATCTCGCTCAAGGCGAGCTTCATT
TAACGCCTTACGCAGTTGCAGAATTTCAGATTCCAGTTCAGCCACCGTGCGGGAACCAGGAGTACCGA
GCCCTTTTCTGGCGGCGGTAACCCATTGTCCTAAAGTGCCTTCAGGAAGAGATAATCGGGAAGCGCCTT
CACTGATCGAAAGTTGATTTTCAGGAACCGTTCTGACAGCTTCGGCTTTGAACTCTTTAGAGTAACGTTG
GGTTTTTCTGCTCATTATTAGCTCCTTCTGATGCCATTCTATTTCAGGAAGGAGTGTCCGTTAAACTCAG
GCTACCTCATYMRWWWMMRRWWMMMWWMMRWRMMWWMMMRMRMRMWRRMMMM
RRWWWMMMWMRMMMMMWRWRMAMCGTGGGCGGTGATGGCATAGGCCAGGTCAATATGTT
GCTCTGCCCGCTCCTGACCGGGGCGAATCACACGGGTCTGCTGACCGTCAGAAAGTGTGACACTGTCA
CCGGAAACCGCTGTCACCGTCCAGACGCTGTTGGCCACATAACCGCGCTCCCGGTCACTCTTCGTGAAG
CGTATCCTGTCACCGGTCCCCACCCGGATGGTGTCCGGGGTGTACAGCGTGACGCCTTCAGCCACCGCC
TCCCGGGGCGAAATCAGCCGCGTGTTACCTTCCGCATCCTGCAGGGTTATCAGCCCGTCATCCTTGCTG
ATACCGGCAATCCGGTGATACACATTATCCACCAGGGCAAGGGCATCCGGGTTATTCTCCCAGGTGGA
GAGACGACGCAGCTCCCCGTCGCGTATATTCGCTGTGTTCAGGACAGGCACCATGACCTGCACCTGCCC
CAGTTCTCCGGCCTTTTCCCGTGCATCATGAATCATGCTGTTCAGTACGCGCCGATCCTCATTCAGGTGC
GTGACAATCAGCGTCTGCTCCCGTGCTTCCGGTGTCCTGCCGGTATAGTCGCGGACAATGGCTTCATAC
AGTGTCATGGGGACATCCGGAAAAGCCTCGCCTTTCAGCATCGCCTTCTGCTGCGCTTCTGCCAGTTTC
GCTTCCTGGCTGTGACTGAACTCCGTCACGGAGTGCTCMGGTGCCCATGCGCCCTCCAGACGTGGCAC
CTGAGACGGTTTCACACGCTCAAGCCCGGACAGCGCCCTTTCCACATCCCGGTTAATCAGGCTGTATAC
CGCCTCCCGCAGTTCCGGCGTCTGACGCACAATCTCCTTCATGATGACCACATCGGCAGCACTGCGCGT
CTGCTGGAGACGGAAAGGCTGACCAGGCGCGATGGCCTGCAGCTGGTCCGTGTCACCGCTGGCCACA
GCCCGACCGCCACCGACCGCAATCAGGGCGTATGCCCGTGCCATGTCGGTATTGCCCACCATTGAGCTC
TCATCGAGCAGGAACAGCGTGTTGCTGAAATCCGGCGTTTCTCCGCTGCGCTGCTGCAGCTGCGTGTCA
TGCAGAAAGGACGCCAGTGTCTGCGCATCCACGCCGGCGCTGCGCATCTCCCCGACCGCGCGGTGCGT
GGGACCCAGCCCAACGACACGGGGACGTTCACTCTCCGGCAGCATGTTCACGGCTGACATCACCGCCC
GGAACTGTGTGGTCTTACCCACACCGGCATAGCCCTGCACCACCGTGAAACGGTCGGACGTTTCCAGTA
TCATACGGGTGGCGGCGCGCTGTCCTGATGTCAGTTTCTCCATGAGTTCGCCAGGTACTCTCTCCATCA
GCGGCGTGACCGCCTCCTTACCTTCGAGAATATGGCGAAGAATGCTCTTTTCTGCCTCATACGACGCAC
GGGAAACCAGCAGGCCTGTGCCATAGCCTTTTGCCACATCCACATACAGTAAATCACCCCGTTTTATCTG
CGCATTGATTTCCCCTCCCAGGTCAGCAAAACCGGTTCCTTCTGCAGCAAACGACTTCGCCTCTGTCAGC
AGGTCCACCATGCTGAAGGCCAGGTTTTTACTTTCCAGCACAGGAAGGGCCAGATGAATGGCCTGCTG
TGCCGGCGTGTGAAGCCCGGCTTTCTGCAGACTGATAGCGGTTTCCAGCAATGTCTCACCGGCACGCGC
CTTTATCTGCTCAGAAACCACCGTAAAGGAGGGATGGCGGGCAAGTTTTTCCGCAGTACGGGTTTCATC
CAGTGAGGAATACAGCCGGACATCACGACCACTGCGGGCCAGACCGTTCAGGGTGGCATTGTCCATTG
CCATCTGTGTGACGGAGGCAAAAACCGTCGCACTGTCGCTGACGGAATGCCCGGGCGTTTCCACCCAG
CCGTTCTCCAGCTTCAGTGCCGTGAACGGTGAATCGCTCACAGGCAGGGTGGCCGGTTCAGCCCGTCCC
GGCACAACAACCGTCATCGCATCTTCACTGACGGATGCCACCTGCAGGCGGTCACCGCCGGAGACGCG
GAGTCCGGGAATTTTCCCTGTCACCCTCAGACGCTCACCGTCTGCCACCGGCATTTTTTCCGGCCGGAAC
AGCGACCAGCTGCTGTCCAGGGAAGAAATACGCACCACCTGCGTTTCGCCCTGCGCATTCCGCAGGGT
CAGGCTGTGACTCTGCGCCGTCACACGTTCTGTCACATAGCGGTCATGACTGCGTGTCTCCGGGTTCCA
CTGCTCCATCACCATTCCCGGGCGGTACATATCCCGCAGATAACGGCTCCGGCTGTCCAGCCAGACCGG
TGACAGGGCGGTCATGGTTACCTCCGGGTGTCCGAGCACGCCCTGTGTTTTCAGCTCACTGCGAATGGC
CTGTGTCAGTATGGCCTGTTCCCGTACTCCGCTGACCTGTGCCACGCTCTCTTCTCCGGCTTTCACGCTG
GCCGCAAAATCTCCGGCCAGCCGGGCATAGCGGACATTACGGTCCGGTTCACTGATGATGGTGGCCGG
TCGCTGTTCTCCCCCCTGCCAGCGATATGTGTTCACCCCGGCATCCTTCATGGCCATCAGTGCACTGCCT
GTACCGGTTCGCTGCCCGCTGTMGGTWAWMMGGAMMTGTACGTTATGACGTGCGGCACCATCCAG
CAGGGTTAACGTCTCTTTCAGGGAGAGTTTTTCGCCCTGGTCGACGATAACGGTACTGCCCGGCGTGAA
GGCCATGCCTTCCAGCAACTGACGACGTCCGGTTATCAGTTCACCGGACAGCCGTTCATCCTGCTTCAG
GTTCATCTGCGAGCGACGGTCAGCAGCGATAATCTGCACCTCCCGCCCCTGCTCCCGGGCCATCATGAC
CAGTTCAGCCACCCGCTCACGCTGCCCGGCTGCACCGCCCTGCCCGGACACAATGGCCAGCGACGGAC
GGTCCTGTGCCAGAACACTGACGGCATCGCTGTAACCGGCCGTCCGGGGGACACTTTTCTCCGGATGT
ACGGTCACCCGGTTCTGTTTCATGATGTCACGACTGAGTGCCCGGACTGACAGCTCATCGAGCATATGA
ATCCCGGACGTGAACAGCCCCTTCTCACGATCGAGGGGGATAAGCTGCTCACGGCTGATGGCCTCATC
GATACCGGCGCGTGCCCGTTCAATCACACCATTTTCCGGCGGCAGTATGCCAACCGTCCTGGCCAGCAG
GTCCGTATACATAAACTGCACTTTGCGTTCACTTAATCCGGCAATGGCCTGTGTCACCGCCTGCTGCACA
TCCGGCCCGTCCTGTGAAGCGGGTCCGGGTGTCTGCGTGCGGGTATATGCCCGTTGTTCCGCTGCGTCA
CGATATGCCCGGATGTCGAACCCGGTTTCCTTCAGCGTCTGCATCCACTCGGCCATTCTGACTTCAGGAT
CGACGTGCTGTTTGGATTTACGCGTATCCAGCGCCGCCACATCCCGGGATTTCAGCGAGGCGTCTTCCC
CGACGGCCTCCCGGATAGTCTGTGAGCGTCCGGAAAAGGCCTCCACCGGTACACCCGGCATTTCCCAC
ATACCGTGCTTACCGACCACTTCAGTTTCATAGCCCAGCGCCTCAACCTGCTCTTTCCGTTTTTCCCGGTA
GAGCCTGCCAAAGGCAATCTGATTAGCGTACACATTCTCAATGAACCCCGTTTTCCCCACTTTGTCACTG
CTCAGTGTCTTCCATTCGCCGTTATGCTGCGTAACATTAGTCACCACCGCATGCGTGTGTAACTGTGGTT
CCTGATCGCGACTGGTGTCGTGGTTAAACAGTGCCATCACCAGATTACCGGTCAGCACCGTTTCTGACT
GTCCGTCCGTCATCACCCGTGTGGAGGCCAGTGCCTCCACCTGACGGACAGCAAAATCCACGGCCTGG
TTATGTGCTTCAATAAGACGTTTGTCCCCCCCGAGCATGGCCATCATGGAGACACTTTTGGGAGCGGAG
AAGGTCAGGTCGTAGCCGGGACGATGCCTGTTACTGCCATCCTGCATGCGGCTTAAATCCGCCCCGTCC
GGCAGCCTGCCCTCCAGAAGGCGGGTAAAGACATCCTTATCGACACTGCCCTGCAGCCCCAGTTGTTCA
GCCCCCTGGCCGGCCCAGCGTTCTCCCATGCTGCCCAGCACATAGTAATTATCCTTGTCGGTATAATAAT
TATCGGCACTTCCGGCCGATCTGACCTGCGCGATACTCATCATAAACGTATATCCCTTTGACATATCCCG
GCATCAATCCCACAATAGATATACACAAGACATATCCACATAAGGAGGCAAATAATGGAAACCACCGT

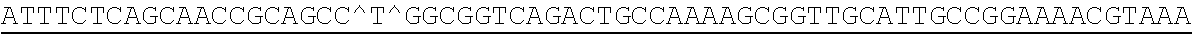

ACGCGTTGAAGTGATTGCTGTCGGACGAACCAGAATCATTACGCCAGCCGGAGAGACATGGGACGAA
TGGTTCGACGGACACAGCGTCAGCGCCGATTTTATGGATAACAGGGAACAGCCCGGCATGCAGGAAA

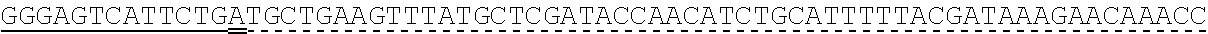

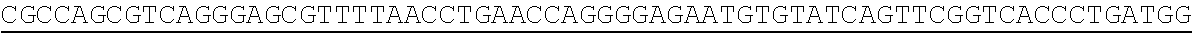

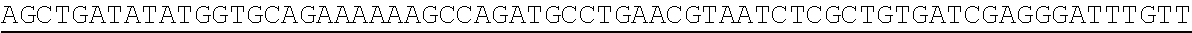

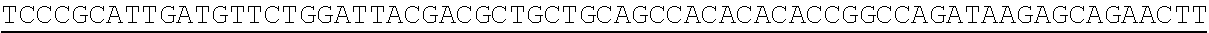

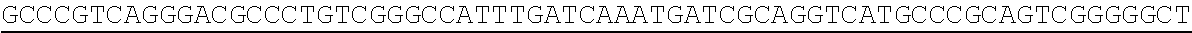

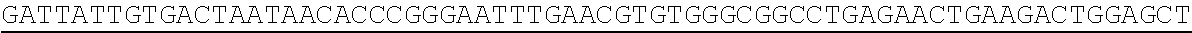

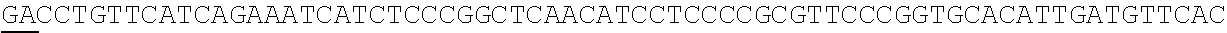

CTCTTCACGACGCTGCATCTGCTGCCAGGTGTCCGGATGATTTTCCTGCTGCCATGCCTCATAAGCGGCC
ATATCCACCACTTCACCGGATTCACTGATCCCGGGTGGCAGTTGCTGTTCCATCTCTTCTTCCGGTTTCAT
TTTCAGCTCCTGCTCAATCCCCCCTGCCGGAACATTCACACCTGAATCTGACTTCTTATCGTTGATGACAG
GAGACACCGGCTGTTGCGGCTGTTGCGGCTGTTGCGGCTGTTGCGGCTGTTGCGGCTGTTGCGGCTGT
TGCGGCTGTTGCGGCTGTTGCGGCTGTTGCGGCTGTTGCGGCTGTTGCGGCTGTTGCGGCTGTTCAGC
CTGAGTCACGTCTTCTCCGGAAACAACCTCCGGGACATCCGGTTCGAAGAGGCTGGCCATCTGACGACC
TTCTGCTTCCCTTGCGGCAAGTACGGCACTCAGACGGTTCTCCATTTCCGGGTTGATGTCACGCGGAAT
AAACTCCGGAGCGACCTTCGGACGTTCCTGATATTTCAGTGAGAGTTTGACTGCCGGATACGGTCCGG
GCAGGGTGACATAGCAGGTCAGATCCGGCAGGGACTGAATGTCGGAATAACTGACCAGCGTCTGGCG
CTCCATATCCTTACCGGTCGATACCCCGTCACGTACCGGATCAGCACCGTAGGAATACTGTTCGCTGGCT
TTCAGGTGCTCTTTCTCACCAATTTCACCGACAGCGAACTCCGCAATCTTATGGCTGGGAGAACGGAAA
AAGGCACGGGTGTTCATGACGTCAAACAGCGTGGCAGCCGCTTTCTCACCGTAGATATCTTCCAGCTGG
GCATAGGACTGGATACCAAACACATAACAGCCACCGAACTTACGGGCTTCCGGCAGGATCTCCACCAG
GTCCGGCAGTTTGTGTAATGTGGGTAACTCATCACAGAAGAACCACACACGACGATTACGGTTTTCTCC
CATTGCCAGCAGACCACGAATGGCAATGGACAGCCACATGGAGATCACCGGCTTCAGGGAGGCATGA
GTGTCGGCATTCGACGAGATAAACAGCCAGCCGTTTTTCTGATCCTCCCGGACACCACGCATCCAGTCA
CGGATGGTGAAGGGCTCACCGTTATGCTCAATCCCCTGCAGGTAACGGATGGCTTTCACATAGTTGGTC
AGCACAGCACGGATGGAAATCGCCGTTTTCTCAATTTTCTCTTCCACCAGGTTGGCCGCCGGTGAATTA
CGCAGGTAGGTACGCAGTTTTTCAATTTTGATGGAAAGCAGTGTGTCCACCAGTTTGCTGTAGCTGCGG
TTGGGGTCATTACGCATCAGATACGCCGCTTCCGCAAAAATGGTACGTCCTGAACCCTGCCAGAACGG
GTCCTCTTTTGTGCCCATCGGGATCAGAGTATTTGCGGTATTATCAAAATCCGGCTGTGTCAGGCACTCC
TTCCACAAATCCCAGGCGGCACAGCGTGCATCCAGCGGATTCAGGATCTTATCGATGGAGGGATCATA
GTAACTTTTAACAAATTCCCCTGAACGGTCATAAATCACCACCATATCACCACGCTGACGGGCGTAGTT
GGCCAGACGACGGATAACCTCCGACTTACCGGCCCCGACCGTGCCGTGCAGACAGAAGTTCTGGATTT
CGGAATCCCGGATAATCGGCAGATCGCCAATCCGGATATCGGAGTCCCTGCCGTCTTTTTTCAGCATCC
GGGCAACGTCTTTCGGATTGTCTGTCAGTTGACGACCACCTGTGACTTCATTTTCGCTCTGTTGTTTACC
CTGACGCCCCAGAATCCAGGAGACAACAAAGAAGGTAATCAGGCAAATAACCAGTGCCACAACTGTGG
CCAGAACGAATGCAGACCATAACTGCTCACCGCACCAGATCATATATTTATCATGCAGTACCTGAGCAG
CGTTCATCCGGAAGGTTTTGCCGTAATACTGAATCTCATATACCGGCTGAGACTWGAWWMMMTMM
MGMATGCMTTMMMGCGTGGTACACMACCAGTMMMTACAGCCATTCACAAACGTCTGCCAGCTTA
TTTTTACCCMWMMMMMMMRMMMMMMRMRWMWMMMRMMMMMMMWMMMMMRR
MRRWMMMRMMWMMWMWWRRSRMWMWRRMWRMMMMWRMRRMWMMRMRRWMM
WRRMWMMMMMTWATTGATAGTGTTTTATGTTCAGATAATGCCCGATGACTTTGTCATGCAGCTCC
ACCGATTTTGAGAACGACAGCGACTTCCGTCCCAGCCGTGCCAGGTGCTGCCTCAGATTCAGGTTATGC
CGCTMAATGAGGTGTACTGGCAATAGCGGACACTACCATTTGTTCTTTTTTTAAGCAGCCATCTGATGA
TATTTTTCCCTGAAGGCTGCCGGGGAGATATTCCCCAGACGAGAGTGACGACGCTGACGATTGTAGAA
AATCTCAATGTATTCCCGTATTACTGAGATGGCTTCATCCCGGTTATTAAAACGATAGTGGCTCAGGCTC
TCATTTTTCAGCGTTCCCCAGAAGCTTTCCATCGGAGCGTTGTCGTAACAGTTACCTTTACGCGACATTG
ATGTTTTCAGACCAGACTGCTCCTGTATGACCCGGTAATCGTATGCGCAGTACTGTGAACCTCGATCAG
AGTGGTGGATTAGCCCGGCAGGTGGGCGCTGGCTCCTGAGCGCCATAAACAGGGCTTTACCTGTCAGC
TCTTTTGTCATGCGCTCTCCCATGGCGTAGCCGACAATTTCGCACGTATAAACATCTTTGATGCCAGCGA
GGTACAACCATCCCTCCTGTGTGGCAACATACGTCAGGTCCGCCACCCAGACCTGATTTGGTGCTGTAG
GAGCGAACGTCTGGTTCAGCAGATTTGGCGCAACTGGCAGATTGTGGTTCGAGTTCGTAGTCGCTCTG
AACTTGCGTTTCTGCTTACAGCGTAGCCTTAGCTCCTTACGAAGACGTGCCAGTCGGTCACGACCAACG
ATGATGCCATTCTCTGCCAGCTCCGTCTGGAGCCGCCGGGTTCCATATGTTTCGCGAGTGCGGATATGT
GCCACCTTAATCTCCAGTTTTAGCCGCTCATCACTTTGTTTTCTGTCTGAGGGTTCATGCTGTACCCAGTT
GTAATAACCGCTCCTGGATACACCAAATACCTGACACATCGCTTCAATGGGAAATTGTTGTCGCCATTGT
TCGATTAACGCGTATTTTTCAGCGACTCCTGTGCAAAATACGCTGTTGCTTTTTTTAATATATCTCGCTCA
AGGCGAGCTTCATTTAACGCCTTACGCAGTTGCAGAATTTCAGATTCCAGTTCAGCCACCGTGCGGGAA
CCAGGAGTACCGAGCCCTTTTCTGGCGGCGGTAACCCATTGTCCTAAAGTGCCTTCAGGAAGAGATAAT
CGGGAAGCGCCTTCACTGATCGAAAGTTGATTTTCAGGAACCGTTCTGACAGCTTCGGCTTTGAACTCT
TTAGAGTAACGTTGGGTTTTTCTGCTCATTATTAGCTCCTTCTGATGCCATTCTATTTCAGGAAGGAGTG
TCCGTTAAACTCAGGCTACCTCAGGGTCAGTMMWRMMWMMMMRMWRWMRWRRWMMMRY
MMMMMMWRRMRRWWMMWMMMMMRRGARGTMAGRMWRMWRMMWRMRRMMWMM
RMRMWMMMSRWMMRRWMMMMMMMRWWWRMMMMRWWMMYMMWRWRRMRMMR
RWMRMMMWSMRRMMWMMWRWMAMMGMMAMMRTMMRRMMRRMRRAWRMMRTRM
MMRMMKMMGCCYTSATATTCCGGCATCATTTTWTKCGCCAGCRMRTMYACMKACGCYWCRGCAW
MMMGAMMATCSWKRMMMMMMRRRWMMRRYMRMMMRRMSMRMMRRWMRMMMWMW
WMMMMRWWMMRMRWRMMMRRWWWMRMMWMWWRMMWRMWRKWWWCMTMCGT
TGTWMMMMWRWWMYRMRMMAMMRRRMRRTGCCGTGCCTCCATCTTWGCCGGGCSWTAAGG
CRGGGATGCACARRRCRCMMRRRMMRMMRMMRMMYRMMRMRMMMKRMYRRMRRRARWWR
RMRRRWRMRMMMRMRMMMWMKRMRMRRRWRMMMKMMMRRMMMMTCACRRCAGACRR
GAARGARRGACCGGGTGACCGCAGCGGCGAAGCGTACCCTTGCATCAACGCCAGCCCGGCTAMGATG
GAGGCACGGCACARMMMGCAMMCGRCGCTGAGGCYACAKARCTGAAAACCCACAAGGCCATGCCC
CGCATGGCCGGGTAAAGCGGAGCCKGCGGCGACWTCCTTTGTGCTCTKTCCGGGGACCGGCWGGAA
AAAGGCGGGGATTTACCCCATAAGCGCTAACTTAAGGGTTGAACCATCTGAAGAACGCGACGCCTCGG
TGCCTCGTTAAGACGATGCCTCGCGTTCTTCAATTGCGTTTTGTAGGCTGTCAGGGATACTGTCCCACGA
ATGGCCACCTGTAAGCTCCAGATGACCATTTTTGTTATTCTCCACAACGCGTTAGTTCTTCTTTTCGGATC
CGGCACTTCTGGGGGGAAAATCCAGCGATGGCTGGATTATGTCGTCAATTAAAAATGCGGCGAGTAGA
TTAGCAAATATCCACGCTTTCGCGAGTTCAAGTTCCTTTGCACGCAAAGCATCCAAGTGCAGCAAACTTT
TGAGCCGCTTAAAAGCCAGTTCAATTTGCCATCGCAGACGGTAACAATCAGCCACTTGCTCTGCTGAAT
ATTCATCTTCCGATAATGATGTTAGCAATAGCACATGGCCCGCTGCTTCCAGCGTTTCCGCCTGAACTAC
TCGTCCTTTTCGACGATTCTCGCTGAGCAGTCGGGTTTTACTGATTAATGCTTTTTCGGGAGGAAGTGAT
ACGGCAATGAGACGTGCAGGAAAGGGAGCTCCGGCTTTTTTATTACCTGAATTGCCTATCATTACAGTG
GTTTCACCGTTCTTACCGCAATCCAGCCCGCGCAGAAAATCCATCATGTCAAAGCGCATTCCTTCTGCAG
TTAACCAGCGCAATCCTCGCCAGTAAACCCGGACGATATAATCAGCTTCTCCAAAAGCAAGTGAGCGGA
TACATTCGGGACGCGAACCGAATCCCCGGTCAGCAATTCGTATCTCGTCTGCCGTTTGCGCAAATCGAT
CCAGCCGTTCAGCGTCTCTGCTGTCGGTTAGCTCAAAATCAGTGAACTGACAGGTATGAGGATCATATC
CCATATGTAGTCGCCATTCAGCGCTGCCGCCCCCGGGCGCGCTGATTGCTGTTCCATCGACAAGACGCA
ATCAAGGTGCGGCGGGAGTCGATGGCATGCGCGTAAGTGAACTGCCGGACTACCTGAGGCACCACTG
GCCAGAGCTGAAAGCGCAACTGCTGTCCGGCAGATACCGCCCATCCCCTGTGAGAAGAGTATCCATAC
TGAAGCCCGGCGGCGGCGAACGTCTGCTGGGCATCCAGATGGTGGTGGATCGCTTCATCCAGCAGGCT
ATGATGCAGGTATTGCAGGCACTGTGAGATAGCTCGTTCAGCGACAACAGCTACGGGTTCCGTCCCGG
TCGTTCGGCCCACCAGGCAGTGATACAGGCCCAGGAATATATCGGGGCCGGGTATCACTGGGTGGTCG
ACCTCGATCTGGAGAAGTTCTTCGACCGGATAAATCACGACGTGCTGATGAGCCGGATAGAGAAACGA
GTATCGGATAAACGGGTGTTGTCACTTATCCGCAGGTTCCTGAATGCAGGGGTAATGGACGCAGGTCT
GGTAAGGCCGGTGACAGAAGGAACGCCGCAGGGCGGCGTGATCTCGCCCCTGCTCTCAAATCTGTTCC
TGCACTATGCCTTTGATATGTGGATGCAACGTCAGTGTCCCGACGTTCCCTTCGAAAGGTACGCCGATG
ATGGTGTGCCACGAAGGCGCGCACCAGGGACGTGAGTCCCTGTGCAGCCATGCTGCACAAGAGATGA
GGGAAGGCCCCTCGGTGTCGGCGTGCAGGCGCAGGCACCAAACCACCCACTGCTGCTGCATTTAAGAG
ATGTGGTGGTGAGCGAGATGGGAAAAGGTCGGACGTGAGCCGATCAGGTATGGATCGAAGAGACGA
ACGAAAGTGAACCGCTGATGATGTGTCGAAAGCATATAGATGATGTCAAAACCATGGTTATCCGGCTC
CGGCGTTAACGTCACGGGAAGCAGTGCCGGCATATTTTTTCCGGAAGGCAGCAGGCCACCTTTCCGGT
ATTGATGGCGCCAGTTGAAGAGCAGGTTATCGTTGATTCCATTTTCACGGGCAATCTGCGCCACACAGG
CTCCGGGCTGCAGTGACTGCTCCACTAAGGCGATTTTGAACTCATAAGGGAAGTTGGGGCGCCGGGGA
CGCTTTTTTACCCCGGGGACTTCGGGCATAACGGTACTTTCAGTAAGTACGACAGGTACTGTGGAGGCA
CTCCCGTAAAGACGGGCGTCAAGTTCCCGCTCCGACATACCTGCGGGCAGAGGCCATGAAAGGCCAGC
TTTGCGGAAGCGCACAAACATACTACAAACTGTTGATTTTGGTACGCCCAGGCGACGTCCGGCTACAAC
GAGGGGTAAATGTTCTTCAAAGTGAAGGCGTAAAGTGTGGATGGTGCAGAAGATGCCGATTTCCAGCC
CCCGTTTTCGGGCTGCATACAGCAGATACTCCACCGCCAGACGACACACGTCATTGCAGCCGTCAAGCT
GCCGTGRTTCMGTGCCGTTCTAAATAGCTGCGCGGAATAGTAGATCACTTTGAGGGAACTTAGCCCGG
ATTGTGCGATCTGATCAATCGCCAAATCAAAACAAATCACCAACCGGACTGAGCAATGCCGATCATAGC
ACCAATTTCCCGTGACGAACGACGCCTGATGCAGAAAGCCATCCATAAAACACACGATAAAAATTATGC
CCGCAGACTGACTGCCATGCTGATGCTGCACCGGGGCGACCGTGTCAGCGACGTTGCCAGAACGCTCT
GCTGCGCCCGCTCCTCTGTTGGACGCTGGATTAACTGGTTCACGCAGTCGGGTGTTGAGGGACTGAAA
TCATTACCTGCCGGGCGTGCCCGTCGCTGGCCGTTTGAGCATATCTGCACACTGTTACGTGAGCTGGTA
AAACATTCTCCCGGCGACTTTGGCTACCAGCGTTCACGCTGGAGTACAGAACTGCTGGCAATAAAAATC
AATGAGATAACCGGTTGCCAGTTAAATGCCGGAACCGTTCGCCGCTGGTTGCCGTCTGCGGGGATTGT
GTGGCGAAGGGCTGCGCCAACTCTGCGTATCCGTGACCCGCATAAAGATGAAAAGATGGCAGCAATCC
ATAAAGCACTGGACGAATGCAGCGCAGAACATCAGGTCTTTTATGAAGATGAAGTGGATATCCATCTT
AATCCCAAAATCGGTGCGGACTGGCAACTGCGCGGACAGCAAAAACGGGTGGTCACGCCGGGACAGA
ATGAAAAATATTATCTGGCCGGAGCGCTGCACAGCGGGACAGGTAAAGTCAGCTGTGTGGGCGGCAA
CAGCAAAAGTTCGGCGCTGTTCATCAGCCTGCTGAAGCGGCTTAAAGCGACATACCGTCGGGCGAAAA
CCATCACGCTGATCGTGGACAACTACATTATCCACAAAAGCCGGGAAACACAGAGCTGGCTGAAGGAG
AACCCGAAGTTCAGGGTCATTTATCAGCCGGTTTACTCGCCATGGGTGAATCATGTTGAACGGCTATGG
CAGGCACTTCACGACACAATAACGCGTAATCATCAGTGCAGCTCAATGTGGCAACTGTTGAAAAAAGTT
CGCCATTTTATGGAAACCGTCAGCCCATTCCCCGGAGGCAAACATGGACTGGCAAAAGTGTAGCGGTA
TTAAGCGCAGCTATTTAGGCATAAATTAAATTTAACGAACTGCATCTACACAGTCCTCCGTGCCAAACG
GGCAACACACGGAAGGGGGATGTGTTTCGATTGGTAACCTGTAAGAAACAACTAGTGGAAATACCTGT
ACTTTATAATTTCAAGGGTACGGGTCCGCTTCGTTTGATTTGCGCGTTTTTAACAATTTTAAAACCCAGA
TATACTGTTCCGGATGTGACCCCACCAAAATTTCCACTGTTTTGTTCATTTGTCGGGCAATCATTTTATTG
TCCGCGCTGGCAATGCATGCATCCATTGGTGGCCGAACTTCAATAGTCAGGAAATGTTTTTTTTCATCAT
AAACCGGGAAAAGCGGTATAATCATAGCCTGACTGATGTTCATCAGACGTCCAATAATTGGTAATGTCG
CTTTATAGGTCGCAAAAAAATCAGCAAATTCACTGTATTCAGGACCATGATCCTGATCTGGAAGGTAAT
ACCCCCAGTATCCCTGGCGTACTGACTGAATAAATGGTITTATCCCATCCTCCCGGGAATGTAAGCGCCC
CCCGAATTTACGCCGTACAGAATTCCAGACATAATCAATCACTGGATTTCGTTGCTGATGAAACATGGC
GGCCATTTTTTCCCCCTGAGCTGCCAGCAACATTGCAGGAATATCCACGCTCCAGCCATGGGGAACCAG
AAAAATCACATTTCTGTTATTGTGCCGGATTTCCTCAAGAATTTCGAGGCCTTTCCAGAACACTCGTTTCT
GGAACTTTTCCGGACCACGAATTGCCAGTTCTGCCATCATCACTATGGATTGCAATGCTGTGGCAAACA
TATTGTCGACAATTATTTCCCGTTCTGTATCACTCTTTTCCGGGAAACAAAGCGATAAATTAATCGTCGC
CCTGCGACGAGCTTTTCTGCTCAATCTGCCCACCCAACGCCCTATTTTGGCCAAGACAGGATCTCTGAAT
GAAGGGGGAAACATTGAAATACCTGCAATCATTCCCAAAACGAACCATGTAAACCAGTAAACAGGGGA
AAGATAATTTTTCTTAAATTCAGGAATAAACTCGGATTTGTATTTTTTCATTATTTAGTGCGAATGCCCCT
ACCAAAATCATCATTAATATTTTATTCAGAGAATACATCAAACCTTAATTTAAGTCCTGATGTTCTGAGTT
TACAGGATATGCATCCAAAGAGCGGATAGCAGTTTCATGGATATAATCCAACAAAGCGTATCGTTTTCG
ATACTCAGAGCGTTTTTTACTAGCTATATTGCTTAGCGGCTTTCGAACCACCTGTGTTGGTAATTTGTAC
CAGTCACCACAGGTTTTCCCTGCTACAGACTCCCAGAAATCACTGTAGACAGCAACAAAGGTTTTGCGT
TTCTGATACCAGTATCTCAATTGCCGGAAAACATGACTGTGCTCACTGACTGCCAGACACTCAGAGACA
TTACAAGCTTTCATCAACGCACAAAATGCTTCAAAAATAATCCGTTTTGGGAAAAGCCCATAACAGGCC
CGGGTTGCGCACCGAATAACATCAGGTCCGGTATTTTTGGGGGCTCCCTGTAATCCGCCAATAAAAGCG
GTGTGTCCATTTTGAGTCAGAATGACTGAAAATGTTAACCGGGCAATCACTATTTTATTGAAAAACAAT
GACAACATCAACTCTCCTTCCCGGTCAAAACCACTGGGAGAACACTGGATATCAAAATCCTCTCCATCTT
TCCCGATGAATTGGACTAATGATACCGATTTTTGTGGAAACAAAAATTCAGAATATTTTTCTGGCAAATT
TTGCACAAAGCGATAATGCTCAAGAATATATTGCCCCCGGGACCAGGCTCGCCCCCCTTTATGCAAATA
AGGTCGATGAATTCTTGCTGGCAACGTGGGTTGTATTGCCAGAATATCCTCAATGCACTGCAAGGAACG
CAGTTCGTAATAATATCGAATACTGGCAACCGGATTTATAGCACATCGCAAAATGTACATGAACCGGAA
TTTTCTGTGTTCCCACTTCCCCGGGAGTGAATCTGAGGAAAAAACAATCCTTTTAAGGAAACCGATAAA
TGATAAGTTACTTACAGAAAACATATTGTTACTTGTGCTTCGCTAATGTGAGTATTTTTAACGCAGTTTTA
TTAATATCAAACCGCTCTTCTATATCCTGTTTTGCCTGGCGAGCCATCTGATAAAAACGCCCAGGGTCAT
TAAAAAAATCATTCAGGGCGCACATCCATGCGTGCTTATTACCTGCTGGTAACAATGTCCCGGTCTGGT
TGTTCTGAATAACATCAGGGATTCCACCAATCTGACTGGCCAGTACAGGCACAGAAAATGCCGATGCTT
CTGCCAGTACCATACCAAACGATTCGTTTTCTGAAGGCAGAACCACCAGACTGGCAACCCGATATACGG
GGGCGGCAGGAAAAACATTGTCAGCAATAAAAACATCATCATGCATGCCCATACTGTCAATCTGATACT
GTAAATGCTCCCGCAGTTCAGGCGAACCAGACCCCACAATCAACCAACAGAACTGTCGTCCATTCATTT
TTAAATGGAACAGTAAATTCAACATAAATTCGTGCCCTTTTTCCGGGCGCAACATTCCTACCTGAGCAAT
AACGGGGCATCCCCTTCGGGACGCCAGCCAAGAAAGAACATTTGGAGGCAAACTGTTTCGGGAATCAA
CGTATAATTTCTGGAAATCAAAGCCTGGAGGCACAACAGTAACCCGGGTTCGACATCCTTCCTGCTCCA
GATGTGTCTTCATACTTGTTCCGGGAACAATCACCTCATCGCAAAAATGATTTATTGAAAAAACTTTTGT
TTTTCGTGTCAAATATGTCTTTTGCCTGATAATTCTGAATGGATGTTTCCAAGTGAATAACCGTACTAAA
CCAACAATATTGCTATCATGCCCACTGTGACAAACGATTGCATTGGGCTGAAAACCATGAACTATTCCG
AGTAATCTCCATGCAGTAGGGATGTGAAGACTGTTTCGAAATAACGCGAATGTGATATCAATTCCCAAT
TTACTGGCTTCAAAAGCAATTTTGCTGTTTTCCCTGCAGACAAGCAGAACTGAATGCCCCATTTTCTTCA
GGGCCTTCATTTGAGCAACAGCCTGTAATTCCTGACCACCTATATTCGGTGATGATTCCGTAAATAGTAT
ATTCATTATTAATCATCCGGTAATCTTGGCCCCTTATGAAAGGCCAGCAACGATGAAAAAAACGGTGTT
GTATAATAAAAAAGTCGTCTTTTCAGCCATGCGCAACTTTCCCGTTCTTTGGTGCTTATTCTTCCTATCCG
TGTAGTTCCTTTAGCAGGAGCATTCATTCTTCTTTCTGTTGTATATAAATAATAGAATCCAAGTTCTTCAG
CGACCTGAATATAATCCTTGTTATAATAACCTTCCGGCCAACACAAATGTTTACTGCATTTTCCTGTCATT
TCTTTCAAATATTCTCTCCCGCTCAACAAGTCCTGCCGAAGATGTTTACACTGTTCTTCTCGTGAACTGAA
CTTTTTATCCCAGCGTGTATGAGTATGCGTATGAACATGAAATTCGACCAGACCAGATTGCAACATTTCG
TTTACTTCAGACCAGCGAAGCATAACATTATCAGCATTCCCTGTCGCTATTTGATGTTCGCAATCGCGAT
GAGAATATTCCTTTCCAGGAGAGTGACGAACCGGACCATTCCCAATAAAACCGGTAATTAAAAAAATAT
GTGCTTTGAGATTAAACTCTTTTAATAAAGGATATACCTGAAACCAGTTGTCAAGGTATCCATCATCAAA
TGTCAACATAACGCTTTTCCGGGGGAGTTTTCCGCCCCGATAAAAAAACTCCAGTTCATCTGATGACAG
TGTTTTCCAGTTGTTTTCAGCCAACCATTTCATTTGCTTCCGGAATGTCACGGGAGAAAGTGTAACCAGT
CCGGGGCAATGACTGACATGATGATACATTAATACCGGAACATGATTGGCTTTGAACAAAATGCCCCCT
TCGTTTAACATTTAAAGTTTCTGGCACCTAGTAAGCAGAGACAAAAGGAAAACCCAATAAAGCTGGTGC
ATTTTATTTCCAGATACTGGATGAATAACATAGCAAACCCATGGCATATTTGATATAATTTACTAAATTA
AACATCAACAATTCATAAACAATGATATATTATCATATCTTTTAAAAACGACTCACTAAGAACCTTGAGG
TGGCACACTGAATTTGGCTACCTGAACAGTGAGGTGTACTGGCAATAGCGGACACTACCATTTGTTCTT
TTTTTAAGCAGCCATCTGATGATATTTTTCCCTGAAGGCTGCCGGGGAGATATTCCCCAGACGAGAGTG
ACGACGCTGACGATTGTAGAAAATCTCAATGTATTCCCGTATTACTGAGATGGCTTCATCCCGGTTATTA
AAACGATAGTGGCTCAGGCTCTCATTTTTCAGCGTTCCCCAGAAGCTTTCCATCGGAGCGTTGTCGTAAC
AGTTACCTTTACGCGACATTGATGTTTWMRRMMMRRWMYRMWYCWSTATGACCCGGTAATCGTAT
GCGCAGTACTGTGAACCTCGATCAGAGTGGTGGATTAGCCCGGCAGGTGGGCGCTGGCTCCTGAGCG
CCATAAACAGGGCTTTACCTGTCAGCTCTTTTGTCATGCGCTCTCCCATGGCGTAGCCGACAATTTCGCA
CGTATAAACATCTTTGATGCCAGCGAGGTACAACCATCCCTCCTGTGTGGCAACATACGTCAGGTCCGC
CACCCAGACCTGATTTGGTGCTGTAGGAGCGAACGTCTGGTTCAGCAGATTTGGCGCAACTGGCAGAT
TGTGGTTCGAGTTCGTAGTCGCTCTGAACTTGCGTTTCTGCTTACAGCGTAGCCTTAGCTCCTTACGAAG
ACGTGCCAGTCGGTCACGACCAACGATGATGCCATTCTCTGCCAGCTCCGTCTGGAGCCGCCGGGTTCC
ATATGTTTCGCGAGTGCGGATATGTGCCACCTTAATCTCCAGTTTTAGCCGCTCATCACTTTGTTTTCTGT
CTGAGGGTTCATGCTGTACCCAGTTGTAATAACCGCTCCTGGATACACCAAATACCTGACACATCGCTTC
AATGGGAAATTGTTGTCGCCATTGTTCGATTAACGCGTATTTTTCAGCGACTCCTGTGCAAAATACGCTG
TTGCTTTTTTTAATATATCTCGCTCAAGGCGAGCTTCATTTAACGCCTTACGCAGTTGCAGAATTTCAGAT
TCCAGTTCAGCCACCGTGCGGGAACCAGGAGTACCGAGCCCTTTTCTGGCGGCGGTAACCCATTGTCCT
AAAGTGCCTTCAGGAAGAGATAATCGGGAAGCGCCTTCACTGATCGAAAGTTGATTTTCAGGAACCGT
TCTGACAGCTTCGGCTTTGAACTCTTTAGAGTAACGTTGGGTTTTTCTGCTCATTATTAGCTCCTTCTGAT
GCCATTCTATTTCAGGAAGGAGTGTCCGTTAAACTCAGGCTACCTCACAKMRRWRMYMWSYWMRM
MWMMKWMMMMSMMMKSKRMWMMMMWRMMMMMMMRRMMWWWYMRMRMMRMR
WWWMTTTACGGGGTGACGCTTTCATTAACCTGTACACGAACTTCTATTCCKGCATCRTGACAGGCCK
GCAGCYACTGCGCCAYTTCMAGMRGATMGCMMTMMMGGCGTACMACCMMRCCTTCTTTATTCCA
TAACTGCAGAGCATTTTTCTGATTGTTCCATCTGATTTCGGGGGGTATTCCGGGCAAGGTTGTTCAGTA
AGGCATTCCTTCGTAAAATCAGCATGTTTTTTGCACGACGGAAACCGGCAGCCATGAAAATCTGTCTTT
ATCATACTCTGAATCCTGACACGATCCCGGGTTATAAAAAATTTGCACAGGCTATTGCCACCGATAACTT
TGTTCAGGCTGATGTTCGTAAAATTGACACCAATCTTTACCGCGCCCGGCTCAGTATCCGGGACCGATT
ACTGTTTTCGCTGTATCGCTACCACGGTGAAACTATCTGCCTTGTTCTGGAATATATTCGCAATCATGCC
TATAACACTTCACGTTTTCTTCGCAGAAACGTGGTGATTGATGAAGGGCGCTTACAACAACAACCAGTG
CCGGATCCTGTTGATATCGCGACTGAAGCGCTGACGTATATCAATCCGTCTCATGGGCGTTTCCACCGC
CTGGATAAAATGCTGTCCTTTGATGATGATCAGCAGGCACTGTACGAACACCCGCTACCGCTGGTGATT
GTTGGCTCGGCGGGAAGTGGTAAGACGGCGCTGGTACTGGAAAAAATGAAGCAGGCTGCTGGTGATA
TACTGTATCTCAGCCTTTCCTCATTTCTGGTGGAAAAAGCGCGTACTCTCTATGACGCTTCAGGGGAAG
GGAGTGAGGTACAGAATATTGACTTTCTTTCGCTGACGGAATTTCTGGAAACTCTGCGCATTCCGAAAG
GCAGGGAGGTGACTTTCAGTGCGTTCAGCGACTGGCTGCCCCGCAACAGGGCTATCGCCGCACTGGGG
GCTGCCCATACCCTGTATGAGGAGTTCAGGGGCGTGATCGGTGCGGTTGCCTCCGGGAATGGGCCGCT
GTCCCGGGAGGCGTATCTGTCACTGGGGATACGGCAGTCGCTCTATGGCATGGAGGACCGCCCGACG
GTCTATGTCCTGTTTGAACGTTATATCGCATGGCTGAAACAGTCGCATCAGTATGACAGTAATCTGCTCA
GTCATCAGTATCTTTCGTTGGCAACTCCCCGTTATGATGTCATTTTTGTTGATGAAGTTCAGGATATGAC
GCCTGTTCAACTGCAACTGGTACTGAAAACGCTACGTCATCCCGGTCAGTTTTTACTCTGTGGTGATGCC
AATCAGATAGTTCATCCGAGCTTTTTCTCATGGAGTTCGCTCAAAAGTCTCTTTTTCCGGCAGCAACAGG
GAAACGACACAACCGTTAATATCCTGCAGGCGAATTACCGTAACGGGCACCACGTCACTGCACTGGCT
AACCGGTTGCTGAGACTCAAACAGGTCCGCTTTAGTGCTATTGATCGGGAAAGTCACCATTTTGTCAGA
AGCTGCGGTCAGGCAGAAGGGACCATCCGGTTGCTGGACGACAGGGAAGAGACAAAGCAGGAACTG
AATGCCAAAACCTCACTTTCGAATCGTGTAGCGGTCATTGTGATGCATCCGGAACAGAAAGCACAGGC
GCGATGCTGGTTCAGTACGCCGCTGGTCTTTTCGGTTCAGGAGGTTAAGGGGCTGGAATACGAAACGG
TGATCCTTTACAATATAGTAAGCGCTGCCCGGCAGGCATTTGATGATATTTGCGAGGGTCTTACACCGG
CTGATCTTGAGGGCGAGGCCCGTTATTCCCGTCCGCGGGACAGGCAGGATCGTTCAGCGGAAATCTAT
AAATTTTTTACCAACGCGCTGTATGTTGCGCTCACCCGTGCTACACACAATGTGTATCTTGTTGAACAGC
AGGTGGAGCATCCTCTGTGGAGTCTGCTTGCGCTCACGCATCAGGAAGAGCCTCTGAATCTGCAGGAA
GAGATTTCCAGCCGTGATGAGTGGCAGAAAACCGCACATCTGCTTGAAAAACAGGGGAAGCAGGAGC
AGGCGGACACCATCCGTAGCCGGATCCTGCAGACATCTGAGATGCCATGGCAGATTATCACCGCAGAG
GATGCACGACAGTGGAAACAGCATATCCTCGCAGGTACAGCCGATAAAACCATACAACTGCAGGCGCT
GGAGTACAGTCTGATTTATTCCCTGTTCCCGCTGTATAATGCACTGTACCGGGAAGATTTTAAGCCCACA
AGGCAGCCCAGAACGAAGACTCTGCAGTTACTGGAGCTGAAGTATTTCCGGCCATACTCAATGAATAA
CCCGGTTGCCGTGTTGCGGGATATTGAACGTTATGGTGTTGATCACCGTTCGCCGTTTAATCTTACTCCG
CTGATGTCAGCAGCCCGGGCAGGTAATATTGCACTGGTTCAGTTACTGCTGGAGCGTGGTGCAGATCC
CTTGCTGACCGGTAATGACGGGCTGGCGGCTTATCATCAAGTTCTGTCAGCGGCGGTCAGTACGCCCC
GTTATGCGCAACAGAAAAGTGCGCAGTTGTATACCCTGCTGAAACCTGAAAGTCTGTCACTGCAGGTG
GAAGGACGCCTCATCAAACTGGACAACCGGCAGATGGCAATGTTTCTGGTGATCCTGATGCAGGCGCT
GTTCCACACGCATCTGGGAAGTGCTCTGTTTTTTTCGGAAGCCTTCAGTGCGGCACGACTGGCAGAGTG
TGTGGTTCATCTTCCTGAAGCGTTGCTGCCGGAACGCCGCAAACGAAGGAGTTATATCAGCAGCCAGCT
CTCTCAACATGAAGTTAACAGCAAAAATCCTTATGGAAAAAAGCTTTTTCTGAGGCTGAACCACGGACA
GTATATTCTTAACCCGGGGCTTAAAATAAGGCAGGGTGATGTGTGGCGCGCGGTTTATGAGTTGCAGT
CACCGGAAGATCTGGGGCATGATTTGCAGACTTATCTGCAGGATATGTCACCGGAATTAGTGGATATG
CTGGGAGGGAAGAAGGGTTTTTACGAACGCAGTGAAAAGTCTGTCGGATACTGGGTGGGGGGGATCC
GTCGTGCCGCTCAAAAAGCGTGAGGTTCTGTGCTGACAGGATGTAACCGAACTTCATCTGTACGTAAGT
ACGTGATGTCTCCCGCCCACTTCTGGTTCGGACCACTGGCGTAAAAATCCTGCTCCAGCAGATTTTCTGA
CACGGGCAGGCCATGTGCGCGGTAACTGACCGGGCTGAACTTCCGGGAGGCCTTTGCCCTCAGCCCCT
GACGGCGCAGGCTTGCCGCCACGGTTTTTACGTTAAAGTGGTAACCCTGAGCACGCAGTTCATCCGTCA
GGCGTGGTGCACCGCAACGCTGTTTTGACCGGGTAAAGAACGCTGTCGCAGTGCTGACGGAACTGTTG
ACGAGGGCTTATCCCTGTCCGCCGCTGACACCACGTTCAACAGTTTGTGGACTGTTCGCTCGCTTCCGG
AAATCAGGGCTTTCATGGCCTTTGCCTGCAGGTATGTCGGAGCAGGAACTTGATGCCCTTCTTTACGGA
AGTGCCTCCACAGTACCTGTCGTACTTACTGAAAGCACCGTTATGCCCAAACTCCCGGTGGTAAAAAAA
CGTCCCCGGCGGCCTTAATGCCAGTCAGTTAAGCAACTGACTGGCTCTTTTTCGGGGCTGTGGGGTATT
TCCAGGGCCTCTCCTTTACCACTCTCGGGAAGGCCCTTTCCCTTCTCGTCGGTAATTTCACAAGTIGTCCC
ATACTTGCAAGATCGCGCATCAGCTCCGGTATACGTCCCGGTGAAGCGCCCTGCAATGTCATCAGCATT
CTCATCACCATTCCGCATGATTCTGAGAAACTCAGTTGATTCGGCCAGTAACCTTTCAGATGTTCCGCCA
TTTTAATCATCTGATATCTCACCAGATTATAAGCCAGTAAGACACCCCACAGCTCTTGCTCCACAAGCTC
CGGCTTTTTACTTCTCAGCGTCAGCCTGCTCAGTTGCATCGTCTGTTTTATCTCCCTGTATCCCAGTTCGA
TTTCCCAGCGATGACTGTACAGATCCGCCATTTCTCCTCCGGGGAAGCGCATGGCGTCCGTCATCGACG
TCAGCAGAWRRCMGMCTYKWMMWYWGMGMGTCRYRRTMMKYRRKMRKRYKKYMRYWWMMW
WWMYMMRWMMMKKMYAMWWYKWWMSTSSMYGCRRRMTRKWWWWMRRMKWYMMYRR
MWRMWMRMMYWYMMMYMRWWYYMKRMRSWMYWMMWMWWRYRYWMMYWYKMKRRK
MKKWMKYRWMMRRWRKMSKKKYWYKYMYSMMMRRMWMMMSRYMWWWWMSMRWSMM
MRYRMRWMRYMMSMWKWRKMMMYWRMMRWMMRRKWKYWRYMRMMKRYWYSWWMYM
WMMSYYRMKMMKMRMRMKMRKYWWMKMWSWWMYKMMYYRYKYYRRRSRMWSMRKMMSK
MMRYRRMWRKMYRRYYMKYWSSMYMYSRMMRMMSMKWYWRMMMWKMRRRWMSRKMRM
MRKSKWMMMRRMMYRYRKMYRRMRRRRRRMKSMYSMRYYRWWSWMYKRKRYMYMMRRKRYRY
RMMMRMRMMYMMMRYMRRKKRMYMKMWSKSKMRSKMMSMMMYMSKKSRKMKKYRKMKKK
RSSKYMWKMMMSRKSWRYKMKKYWWYYSWKRMYMSRMKKSRRMYMRMMYMMMKYYMSMRR
MRSWRSYRRRSMYRMMWMRMRRSRMKKRSRKSRMMRMMSSKRMRRKKSYYMKSSRKMRWRMYR
KYMMRKSKRWYMRMRMKMYRKYKMMKMRRYWMYWWMMRMYMRRRMRMYMYKMYMMMRR
KRYMYYRKRMSRKMMYWKMRRRSKRRWRMYSRMRMKKKMRWRKYRKWMSMSWWMMYKMYY
MRSYMRRSMMMMRRKMRMYSMSWYMRRKRKMKWKSWRMYYYMMMRRRRMMRKSWRKKRKW
WMYKMRKRKMWWYKYRMSRRRMYRYMRKWWMRMKRRMYKRYMYMMYRYRMRKRWMMMM
WYSMKRRMMMMMRKRMKSKKKWMYRSRYKYKWMSMSRRMMWYWKRRYMKSKMMMMWRWK
MMWYMRMWRMWMRRMMWWMSKMMGCCGGGGACGTTTTTTTACCACCGGGAGTTTGGGCAT
AACGGTGCTTTCAGTAAGTACGACAGGTACTGTGGAGGCACTTCCGTAAAGAAGGGCATCAAGTTCCT
GCTCCGACATACCTGCGGGCAGAGGCCATGAAAGGCCAGCGTTGCGGAAGCGCACGAACATACCACA
GACAGTTGATTTTGGTACGCCCAGGCGACGTCCGGCCACAACCCGTGGTAAATGTTCTTCGAAATGAA
GGCGTAAAGCTTCAGTGATTCAGGTCCGGTATTCCATACGATAGTGTCCACTTCAAAATGATGGACATT
ATTTTTGTGGAACCGGAGGTAACAAACCAGACGGTTTAAATGAGCCGGTTACGTTTCAGGTGTCTCTGG
CTGGAAATCCGTGCGTGATTCGCCTTGCTGCACAGGTATGCATGTGGTTAAAATTTATCATACGCGATC
GCGGCGGTTTTTCGGGTGGTTTGTTGCTATTTTTACCTGTCTGCTGCCGTGATCGCACTGAACGCATTCT
GGCGGTGCATACAATTAAGATATTACGTTAATATCGCGAGGGAATGGTCCCCATATTCTCATAGAAGAG
CCTCTGGCTTATAGCATCAACAAATACGGCTATAAATATTTCCTGTTTAATAACGAATAAAAATCATTTG
TACGCATTTGTAATCGTCGGTCGATAATCTCTTTATCTTTTTTGGTTGCAGCGTAATATTCAGCATAAATA
TTGCGAAAATCTATTGGGAATAAACTTTCACTTTCTTTATCATACAGCATATTACCTGTATTAAGATCGTA
ATGGATTATACTCAACTCATTCAATTTACATATCAACTGTAGATAAAGGCTCTCTATATTATCAGGGATA
TCAGCTGTATCAATGTCACTAAGGGGAGTCCCAGGCACGCGCAGCATTCGGAGGTACACATCTCCGCC
ATACTGTATAACAACGGATGCTTCATCGCCATAAAAAGCATTAAAAAGCTCAGATTCTTGCCAAGCCAT
TTCCAGAATCTCATTGTACTGGTTGCCAATAAGATCATACTTTTTATACAAAGCAGATGAATCATTCACA
TCTTCAAAGATTTCTGCTGTCGACCCCTGACCTATTAACTTACCGAGAATGGGCTCCGTTACTATGCCAG
CATGAGAATTATTATTCTCAAATGGAAAAGGTGTTTGAATAATGGTAGATGTTATTTTCATGTCATACCT
TTGTTACGATATTTAAAAATCACAAGAGATTTTCACACAAAATAACGTTACACGCTACCTTGTGTGGTCC
AGATGTTCCTTCCACACGACCGTTGCGGCCTTTATACGGCTGCCCTGCCAGGCTGCTGGCTGAATAACC
AATCCGTTCGTCCGGTTCAATGGCGGCCATCAGGTTCTGCTCCGCAATGTACTACGAATGCGCTTCCTGT
CCTGGATGGCCAGCATGGTCCAGAACATMTCCATCATTTATCAGCAATCTTCCTGCTCATTTCATCCCGC
CATGCAGCAGCAGAGGATTCGACCATGTCATGGGGAAGCACATCACCGCATTCGAGTTGCTGACGGCC
TGCCGCAACCTGGTCACGGAACCAAGAATTGTGCTGCCGTTCAACGGTCTGGCGCATAAAATCCCGGA
TTAACTGAGAGCCATTACGGTCCATGCTTTTTGCAGCGGCCATAAAAGCATCCTTCAGTTCTGCGTCGAT
TCTTACACTCATATTAACCTGTGCCATTTGCCACCTCGTCATTACCACGTATATACACAGTATATAACTGT
TCAGATATCGTCACCAGGATATGCCGCGCCAGCGGCATGTAAGGCGGCACTCCGTTGTTGCATATGGC
AGAGGAGTAAAACCGCCGAAGCCCGGCGTAAGCCGGTACGTATTGACAGGTTTCACCTGTCCTATCAC
TGGCGCTGACAGTWWWMWRMRMWRWRMWWMWRMMWRRWRMMRWWRWRMRMMRW
WWWMMRRMMMWMRMMRRRMMRRWMSMWMGTCAGTCAGCTGACGGTAAATCTGCTGTTCA
GTCTCACGCATCACCTGTGCACCGGCTTCCCTCTCCGCATCCGCATCACCGCTCAGACCTGATGCTTTCA
GCCGGTCAGCCACCCTCTGAGRGTACTCATTCTCCAGCATCTCATAYTTCTGCTCTTCTGCCWGCGCCCA
GCGGTCAGCTTCCGTACGCTTCAGTACAGCATGCCATGGTCCCCAGAGGGAGAACCAGTCCGTAAATTC
ATTCTCTTCACGGCTTCTGACCATGGCTTCGGCAGTGCGGAGGTCATTTGCTGTCACTCCCGACACGCCA
TAGAAACGCATTTCCTTCACGGCAGTGGAGAGCTGAAGTTTCTCTGCGAGCATGGTCTGGAAGGCCAG
GTAGACTTCTATCTCATCCACAAAATGGAGAGTTCTGACTTTATCCCGGGCAATGTCCTCCAGAATTTCG
AGGCGGAACATTTCCCTGCCCAGGGAGAGCAGAGCGCCGGTATCATTATCGAAAAGGCCTTCAGATGC
CTGATGGACCAGGAGGGTTTTCCGGAGATTGTTCCATGTGAGCGCGACACGGTCCTCACAGCTCTCAGT
GGCATCAGCAGCAACAGCGAAAGACTGCTGTCGAAGCTCCGCAGAGGCACTGAGTTTTTCCAGCCATG
CAGCGACCTGTTCACGGAATCCGGAGGTATTGCGTGCAGAGACGGTATCGGAAAGGCGGTCAAGGAA
CGCGGAAAAGGTGTTGGCRTGCTCTTCATGTTCAAAAGCATGCCATATCTGTGATACATCAGATTGTTT
GTTTTCCGGGAACCATGCTGTCACGGCATCAGCCAGGGGGCGATGGARWRWMWWMWRWYRWM
YRWMMMWMMWRRMRMMRKMMMWMYRYSRRMMRWRRWMRWMMRRYRMMRMRRWW
MMWMYWWRYMRRRMWWRMMRMRMMWRRRMWGMYMMWRRRWWMKMMMWMMWM
WSWMWWRMMMMWWMMWWCMTCARMTWMMRMMWRMWTTCCGGGATATGACTTATCTG
ATTTCTATCAAAAAAATACTCTCTGACAACGGGATTATTTCCCTCAGAAAATGCTGGTAGCTCAGTGAG
GAAATTACGGGTCGCTCTGAGATTTTTAAGAGCCTGTGGGAGCGATGGTAAGGAGACTATTTCGTTAT
AGCTGATATTCATAGTCAATAACGAATCGGGTAAAGCTGGCAGGTTTTCCAGCTCATTGTGTTGTACCT
GAAGTGACTCCAGCGCAGGAGGTAGCACGGGCAATTCTGATAATTTATTGCTGGCGGAATATAATTTTT
TTAGCGTAACAGGCAGTTCAGGTAGGTTAGTTAATTGATTATATGATACATTGAGCAGCGTTATCTGAG
CTGGTAAGTTGTCAGGTAGCGAGGACAGATTTAAACGGTCCAGTCGAAGTTCATCGGAATTATTGATR
AGACATTCTTTAAGTMGGGAGACAGCCTCATCACGCTCTTCACCGGGGAGCGCTTGTTTTTCCCATTTA
TCCCATGCTGAAAAGTAATCAGCATATGTACCGGAAATAGTGTTATAAAAAGAATTTTGGGRCAATGAA
AAGTTATTATTTATCGGTAACATGATGGRCATYCAGTTAAAGCCGRTGGACAAATGGCTTTGTTAAAAG
AAATAAGTAGGAGTTTTTATGAAGTTCATTCCATGAAATGAACTTCAGCACTTTTTTATTATAATAAGGA
YAAACAAAATTGGCTGTAAAAAAACGATACTGTTTCAGTTAACYGTGAATGGCCTGTTTGCACCGTATG
TGACAGTAATACGCTGCTTCATGATAAGCCCTCAAAAACATACTTATGATATGTATATGAGTATATATTA
ATGGTACAAACCATCAATATCCATAAAAAATACACCATTAATTCGCTATAAGTTTTTAATACAATTTTTTC
TGCTGCGATTTTTTTATACGATAATTAGCATACTTATAACACCGTAAGTAAATTACATGAAAGCAGGCAC
AGCGTGATCCGATTTTTCTGCGTATAACGATTTTCCGGAAGTGTAATACGGTTGCGCCCCGGGCCGGGC
CCCCTTTTCTGTTGTTGTGTACGGGCCCACAGAGAATGGTACCGGAGGCGGGCTCAGAAGTACTGAGT
CGGGTGATCGCGTCAGGCCGCGAAGTACTGACACGGGTATTGGTGACTGGTGATGAAGTACTGCCCCG
GGTATGATTTCAGGCTGATTCAGGTTCTGATTCTCTGTCAGTCCGCGTGGTTGCTGGCCTGGCTGATTTC
CTGTTGTCAGGCGTTCAACAACCGACTGCTGGTTATAGCCCGGTTCACGAACACCTGACGGCAGGTATC
ACTCACCCGGTGAAGGTAAATCCCTCAAGAGGCAGCAACTCTGCCAGTCGCTCCTCCGGCCATTCCGGC
AGACGCGTCAGGACGTCCGTCAGCCAGGCATGTGGCTCCAGACCATTACGTTTTGCTGTTTACAGAAG
GCTCATTATCTGTGCCGCCCGGTTTCCGGCCATCAGTGAACCGGCGAACAGCCACGCTTTCCGTCCCAT
AACAACCGGGCGAATGGCACGCTCGCAGATATTGTTGTCCAGGGGCACCGCTCCGTCACCCAGGAAGC
AGCTCAGTTCATCGCGCCGGTTCAGAATATAGTTAATCGCCTTATTTAATGGCCCGTCGGGCGGACAGC
ACGGCTCCTGCTCTTCAAGCCAGGCAAACAGGTCATTCACTATCGGCCGGGAGTACCGTTATCGCCACT
GCCGTATTTTTTCCACAGGGCGCTGTCAGCGCCAGTGATATAAGACGGTAATTCGCCATTTGGATTGTC
CGCTCCACCCAACATGTTGTTTCCTTAAGGTTCTCACACCAGAAAGGACATCAACATGCTGAGCAGAGA
GGACTTTTACATGATAAAGCAAATGCRMMMRCMRRRMRMRTACTGAGGTGTACTGGCAATAGCGG
ACACTACCATTTGTTCTTTTTTTAAGCAGCCATCTGATGATATTTTTCCCTGAAGGCTGCCGGGGAGATA
TTCCCCAGACGAGAGTGACGACGCTGACGATTGTAGAAAATCTCAATGTATTCCCGTATTACTGAGATG
GCTTCATCCCGGTTATTAAAACGATAGTGGCTCAGGCTCTCATTTTTCAGCGTTCCCCAGAAGCTTTCCA
TCGGAGCGTTGTCGTAACAGTTACCTTTACGCGACATTGATGTTTTCAGACCAGACTGCTCCTGTATGAC
CCGGTAATCGTATGCGCAGTACTGTGAACCTCGATCAGAGTGGTGGATTAGCCCGGCAGGTGGGCGCT
GGCTCCTGAGCGCCATAAACAGGGCTTTACCTGTCAGCTCTTTTGTCATGCGCTCTCCCATGGCGTAGCC
GACAATTTCGCACGTATAAACATCTTTGATGCCAGCGAGGTACAACCATCCCTCCTGTGTGGCAACATA
CGTCAGGTCCGCCACCCAGACCTGATTTGGTGCTGTAGGAGTGAACGTCTGGTTCAGCAGATTTGGCG
CAACTGGCAGATTGTGGTTCGAGTTCGTAGTCGCTCTGAACTTGCGTTTCTGCTTACAGCGTAGCCTTA
GCTCCTTACGAAGACGTGCCAGTCGGTCACGACCAACGATGATGCCATTCTCTGCCAGCTCCGTCTGGA
GCCGCCGGGTTCCATATGTTTCGCGAGTGCGGATATGTGCCACCTTAATCTCCAGTTTTAGCCGCTCATC
ACTTTGTTTTCTGTCTGAGGGTTCATGCTGTACCCAGTTGTAATAACCGCTCCTGGATACACCAAATACC
TGACACATCGCTTCAATGGGAAATTGTTGTCGCCATTGTTCGATTAACGCGTATTTTTCAGCGACTCCTG
TGCAAAATACGCTGTTGCTTTTTTTAATATATCTCGCTCAAGGCGAGCTTCATTTAACGCCTTACGCAGTT
GCAGAATTTCAGATTCCAGTTCAGCCACCGTGCGGGAACCAGGAGTACCGAGCCCTTTTCTGGCGGCG
GTAACCCATTGTCCTAAAGTGCCTTCAGGAAGAGATAATCGGGAAGCGCCTTCACTGATCGAAAGTTG
ATTTTCAAGAACCGTTCTGACAGCTTCGGCTTTGAACTCTTTAGAGTAACGTTGGGTTTTTCTGCTCATTA
TTAGCTCCTTCTGATGCCATTCTATTTCAGGAAGGAGTGTCCGTTAAACTCAGGCTACCTCATACATCGT
CGATATTGCGACTCAGATTGGTTGCTCTGAACGGACGGTCAGACGCTACCTCAAATACCCTGAACCGCC
AGCCAGAAAGACCCGCCACAAAATGGTTAAGCTGAAACCGTTTATGGATTACATCGACATGCGCCTGG
CAGAGAATGTCTGGAATAGCGAGGTTATCTTCGCGGAGATTAAGGCAATGGGTTATACGGGCGGACG
TTCCATGCTGCGTTACTACATCCAGCCCAAACGTAAAATGCGTCCGTCAAAAAGAACAGTTCGCTTCGA
AACTCAGCCTGGATACCAGCTCCAGCATGACTGGGGCGAAGTTGAGGTGGAGGTTGCCGGGCAACGG
TGCAAAGTTAACTTTGCGGTTAATACGCTGGGGTTCTCCCGCAGCTTCCATGTCTTCGCCGCACCAAAAC
AGGATGCTGAGCATACCTACGAATCACTGGTTCGCGCCTTCCGCTACTTCGGTGGTTGTGTGAAAACGG
TGCTGGTTGATAACCAGAAGGCTGCGGTGCTGAAGAATAACAACGGGAAAGTCGTGTTCAACTCCGGA
TTCCTGTTGCTGGCTGACCACTATAACTTCCTGCCACGGGCATGCCGTCCACGCAGGGCCAGAACAAAA
GGTAAGGTTGAGCGGATGGTGAAATACCTCAAGGAGAACTTCTTCGTCCGGTACCGCAGGTTCGACAG
CTTCACTCATGTCAATCAACAACTGGAGCAATGGATAGCCGATGTGGCTGACAAACGGGAGCTTCGCC
AGTTCAAAGAAACGCCGGAACAGCGCTTCGCGCTGGAGCAGGAACATCTGCAGCCGTTACCGGATACA
GACTTCGATACCAGTTACTTCGATATCCGCCATGTGTCCTGGGACAGCTATATCGAGGTTGGTGGTAAT
CGTTACAGCGTTCCCGAAGCGCTGTGTGGTCAGCCGGTATCGATACGAATATCGCTGGATGACGAGTT
GCGGATCTACAGTAATGAGAAACTGGTGGCCTCACATCGCCTCTGTTCAGCATCGTCTGGCTGGCAGAC
AGTGCCGGAGCATCACGCCCCGCTCTGGCAGCAGGTCAGTCAGGTGGAACATCGACCACTGAGTGCCT
ATGAGGAGCTGTTGTGATGCATGAACTGGAAGTCCTGCTGAGTCGCCTGAAAATGGAGCATCTGAGTT
ATCACGTTGAAAGCCTGCTGGAACAGGCAGCTAAAAAAGAGCTGAACTACCGGGAGTTCCTGTGCATG
GCGCTACAGCAGGAATGGAACGGCAGGCATCAGCGCGGTATGGAGTCCAGGCTGAAGCAGGCTCGTC
TGCCGTGGGTCAAAACGCTGGAGCAGTTCGACTTTACCTTCCAGCCGGGCATCGACCGTAAGGTTGTCC
GGGAACTGGCTGGTCTGGCGTTCGTGGAGCGCAGCGAAAACGTGATCCTGCTGGGCCCTCCTGGTGTC
GGAAAAACTCATCTGGCCATAGCTCTTGGCGTGAAAGCGGTGGATGCGGGACATCGGGTACTGTTTAT
GCCACTGGACAGACTGATCGCGACACTGATGAAAGCGAAACAGGAAAATCGGCTGGAGCGTCAGCTA
CAGCAACTGAGTTATGCCCGGGTGTTGATCCTGGATGAAATAGGCTATCTGCCGATGAACAGAGAGGA
AGCCAGTCTGTTCTTCCGGCTACTGAACCGTCGATATGAAAAAGCGAGCATCATGCTGACGTCAAACAA
AGGGTTCGCAGACTGGGGAGAAATGTTCGGAGATCACGTACTGGCAACAGCGATCCTGGATCGGCTG
CTACATCACTCAACCACGCTGAATATCAAAGGAGAGAGTTACCGGTTAAAAGAGAAACGTAAAGCTGG
AGTGCTGACCAAAAACACAACGCCAATCAGTGATGATGAAATGGTGGAAAGCGGACAGCATCAGTAA
CGAAAGTATTTAGCGGGCATGAAAATGGCAAATAACGGTCAAACATCGTGGCGTTGACAGCGCTMAM
RRMTRARWTWMTCAATGCGGTAAAGACCCGCGATTTTTCTCAGCTCCACGCCTGCACGGGGCTCCCT
GCTGGCCTTATACAGATTTGCGAAGCCGCGCCCTGCGTGACTCCAGCAACAGGCTGATGTGATGCCGG
GGTGATTATCCGCCAGAGATTTGTACACGCTGTATCCGTCACTCACCAGCGTACCGCCACACCAGCACT
GCAGCCAGGTCTCAGGGTATCTGAGGGCCCGTCCGGTCTGCGAGTCAAAACAGACGACCGGCGGACC
GCTTCTTTCACCGCTGACGTAAGCACACAGCCAGCCGGAGCAGGATTTACCGCCTTTCCGGGTATCAAG
GAGCCTCAGGGACGTTTCATCTGCATGAATAACATCCCGTGTCAGAAGCTCATGGCGTAGCAGTTTCAC
CAGCGGGGCMAGTGCAGCTCCGGCAACAMMRMMMMWMTMMRMMMWYRWMMWRMMRRR
RMRMWYMMMMMMMMWWYRRRMRMMMWWRWRMWRMWRRYKMWMMMKRRRMMRM
WRRWMRRWMWMWWWRMWRMWRMMMMYMWRRRWMMWSRCTCCAACTTATTGATAGTG
TTTTATGTTCAGATAATGCCCGATGACTTTGTCATGCAGCTCCACCGATTTTGAGAACGACAGCGACTTC
CGTCCCAGCCGTGCCAGGTGCTGCCTCAGATTCAGGTTATGCCGCTCAATTCGCTGCGTATATCGCTTG
CTGATTACGTGCAGCTTTCCCTTCAGGCGGGATTCATACAGCGGCCAGCCATCCGTCATCCATATCACCA
CGTCAAAGGGTGACAGCAGGCTCATAAGACGCCCCAGCGTCGCCATAGTGCGTTCACCGAATACGTGC
GCAACAACCGTCTTCCGGAGACTGTCATACGCGTAAAACAGCCAGCGCTGGCGCGATTTAGCCCCGAC
ATAGCCCCACTGTTCGTCCATTTCCGCGCAGACGATGACGTCACTGCCCGGCTGTATGCGCGAGGTTAC
CGACTGCGGCCTGAGTTTTTTAAGTGACGTAAAATCGTGTTGAGGCCAACGCCCATAATGCGTGCAGTT
GCCCGGCATCCAACGCCATTCATGGCCATATCAATGATTTTCTGGTGCGTACCGGGTTGAGAAGCGGTG
TAAGTGAACTGCATTTGCCATGTTTTACGGCAGTGAGAGCAGAGATCGCGCTGATGTCCGGCAGTGCTT
TTGCCGTTACGCACCACCCCGTCAGTAGCTGAACAGGAGGGACAGCTGATAGAAACAGAAGCCACTGG
AGCACCTCAAAAACACCATCATACACTAAATCAGTAAGTTGGCAGCATCACCCCSSMWKMRWWRWW
MMWMMWMWWKMMWMRWMRWRMWWMMWWWMMRMMRRWMMMMRRMMMMW
MMKRMRMRWMMRMRYMWRWMMRRMRRMMWRMRRTRGWTACATGTAGCACAGGTGTRAAA
CCGCCGAAGCCCGGCGYAAGCCGGTACGGTCGTCCGAATATCACCAGTCCCCCTCCGGCTCTGCGAGG
CCATGCGGCCTGTCAGCGATGAAAATGCCTCTGTGCGCGGTCGGAGTGGTCCCGACAAGGGTTTACCC
GCAGTCGGGGCGTATCTCCGCGTTAGCGGGCCGTGAGGGCCGCTTACGAGCGTGTATGAGAGACGTA
CAGTAGTTTTGTCCGCTCACACCAGTACAAACCAGTCGAAATACCGGTAACGACGATTACTCTTGTCTCT
GATGCCGGTGGACTACGAGATTTTATTTACTGGCGTCCTGATGCGGTCAGGACTGGTATGGAACCTGTT
TATGTGATACTTAATGCTCTTCTGGATTCCGGGCGATTTACGCGTAAACTTATTCTGTTAGGGCTGTCAG
GCTCATTTTCCTATATCTTCGGCTCAATTGTTGCGACACTGGGAATGGGACTTGTTGTCGATTATCTGGG
ATGGGGGGCCACATTTATCGTACTGATACTGTCTGCTGTTTTTGCCATCATTTTCACACTGATGAGTCGT
GAACGCTCACTGGAGTTCGAAAAAGAGTAATTTTTTCAGGAAAAACATAAAATTACTGACTAAATATTT
ATTTTTTGAATCGCAGGGCATTCCGGATAAGGTTGATATCGTGTCACGGCTGAAGACCTATGGATACAG
TATTTCTGGTGTGGAGACGGATGACGGATATAAGGCGCTTGTCCGCGCTTTTCAGCTTCATTTCCGGCA
GAAAAATTATGACGGCATAATGGATGCTGAAACCGCTGCCATCCTGTATGCATTGCTGGAGAAGTATTT
CCCGGGTAAATAAGGAGTGAGTCGTCAGAGCTGCGATATATCCCCTTCCTCCATGGGCTGTCTCTTATA
CACAAATCCCCACCCGGGGATTTGTGATGTCAGATGTCCCCTGTAACACTATGTTTTCATTGCGCTCTAT
GAAGCTGAGTGAGCGTAACGACTGGAGCTGCTTCTGCGGTGCTCCGGTGGCGAACGTGAAGTCATACT
CTTCGAACATTTTCACCGCCGGGAAGGCTACCATTCGGGTATACATCGCCTGTTTACGTTGATGACGTG
CCAGTTTTTCTTCATGAAGCATGATGCTTCAAGAAGGGCACTGTTGCAAAGTTATCGATGAGGTAGCTG
ACCATCACTGCTGTCGGAATCGATCTTGCCCTTCTGTTTCCAGTCGCCTCAGCAAACTATTTTTCAGTTAC
CACCTGTCCTGCGGTAAGCGTAAACCGACCACCGTATGTAGCCATTAGACAAGAATTGGTAACTTAGAC
GCCCATCTGACATAGACGAACACCTAAGTATGGAATTACAGGACTGGCGAAAAGAACCTCGTAAAAAG
TATTCGAATGAATTCAAACTTCGGGTGATGTCTTCAATCGACAGAAGTTTCTGGTAATCTACTGGTATTT
AAGGTTTTCCATGGCTACCGTTACTGTTCATTGTCCGCGCTGTCATTCCGATGAAGTTTATCGACATGGC
CGTAGTTGTTCCCGGCATGAGCGTTTCCGTTGTCGCTCATGTAAACGTGTTTTCCAGCTCACTTACTCTTA
TGAAGCCAGAAAACCCGGCGTTAAGGAGCAGATCGTTGAAATGGCACATAACGGCGCAGGGGGCCGT
GATACCGCCAGGACACTGAAAATCGGCATCAATACCGTCATACGGACATTAAAAAGCTCCCGCCCCGG
CGGATAACATCTGCGCCAGTCGCTCACGCTGATGTTGCCCTTATCTGTGAACTTGATGAGCAGTGGCGC
TTCGTTGGCAGCAAGGCTGCAACAACACTGGCTCTGGTATGCGTACAACACTAAAACTGGCGGTGTGTT
GGCTTACACTTTCGGGCCACGGAATGATGAGACCTGTCGGGAACTGTTAGCCCTGTTTACTCCGTTCTG
TATATATTAGGCCGTTGGTGGTAAGTGGATCACTTTTTAGACTCGAATAGCTGCAGTTCACAAAAACAG
GAATCCGGCTCCGTTTTTTGTTGTCATATCAGAGGGATATTTGTTATGAAGAAAATATAGTGAGTATATA
TACTGTCAGGATACAATATGATTAATGGGGTGTCGTTACAGGGAACTGCTGGATATGAGGCACATACA
GAGGAGGGAAATGTAAATGTTAAAAAATTGCTGGAATCTTTAAACTCTAAAAGTCTTGGTGATATGGA
TAAAGATTCTGAATTGGCAGCGACTTTACAGAAAATGATAAATCCGTCTGGTGGGGATGGTAACTGTTC
AGGGTGTGCGCTTCATGCTTGTATGGCTATGTTAGGGTATGGAGTAAGAGAGGCTCCCGTACCTAATG
AAATATCAGAGTATATGACCGGATTTTTTCATAGGCATTTAGAGCAAATTGATAGTGAAGGCATAGTGT
CACATCCTAATGAAACTTATAGTAAATTTCGTGAGCGTATTGCAGAAAATATTTTACAAAACACTTCAAA
AGGAAGTGTAGTAATGATTTCTATTGAACAGGCCACTCATTGGATAGCTGGATTTAACGATGGTGAAA
AAATTATGTTTTTGGATGTTCAAACAGGAAAGGGATTTAATCTTTATGATCCTGTAGAAAAATCGCCAG
ATGCATTTGTAGATGAAAATAGTTCGGTTCAGGTAATTCATGTATCTGATCAGGAATTTGATCACTATGC
TAACAGTAGTAGTTGGAAGAGTAAGAGGCTTTGCTGATGCTGAGAGCCTGTACATAGATTTGAGCCGG
CCCCTTGAAACGCCAGACAGTGGATGTATCTTAAAATAAGAGATAGGCTTGGATATATGTCTAACACTA
GTTCTAATTTTGAGATGACTGGAACTTTGCTGGGGCTGGAGCTTCGTAAACGTAAAACTCCTCAGGAAA
AAATTGCCATTATTCAGCAGACAATGGAGCCTGGCATGACGGTGTCCCATGTGGCGCGTTTGCATGGTA
TTCAGCCCAGCCTGTTGCTTAAGTGGAAAAAATAATATCTGGAAGGAAGTCTCACTGCTGTTGCTGCCG
GAGAGGATGTCGTTCCTGCCTCAGAACTGGCTGCCGCTATAAAACAAATAAAAGAGTTACAGCGCTTG
CTGGGTAGACTGGCCCCCTGAATCTCCAGACAACCAGTATCACTTAAATAAGTGATAGTCTTAATACTA
GTTTTTAGACTAGTCATTGGAGTACAGATGATTGATGTCTTAGGGCCGGAGAAACGCAGACGGCGTAC
CACACAGGAAAAGATCGCAATTGTTCAGCAGAGCTTTGAACCGGGGATGACGGTCTCCCTCGTTGCCC
GGCAACATGGTGTAGCAGCCAGCCAGTTATTTCTCTGGCGTAAGCAATACCAGGAAGGAAGTCTTACT
GCTGTCGCCGCCGGAGAACAGGTTGTTCCTGCCTCTGAACTTGCTGCCGCCATGAAGCAGATTAAAGA
ACTCCAGCGCCTGCTCGGCAAGAAAACGATGGAAAATGAACTCCTCAAAGAAGCCGTTGAATATGGAC
GGGCAAAAAAGTGGATAGCGCACGCGCCCTTATTGCCCGGGGATGGGGAGTAAGCTTAGTCAGCCGT
TGTCTCCGGGTGTCGCGTGCGCAGTTGCACGTCATTCTCAGACGAACCGATGACTGGATGGATGGCCG
CCGCAGTCGTCACACTGATGATACGGATGTGCTTCTCCGTATACACCATGTTATCGGAGAGCTGCCCAC
GTATGGTTATCGTCGGGTATGGGCGCTGCTTCGCAGACAGGCAGAACTTGATGGTATGCCTGCGATCA
ATGCCAAACGTGTTTACCGGATCATGCGCCAGAATGCGCTGTTGCTTGAGCGAAAACCTGCTGTACCGC
CATCGAAACGGGCACATACAGGCAGAGTGGCCGTGAAAGAAAGCAATCAGCGATGGTGCTCTGACGG
GTTCGAGTTCTGCTGTGATAACGGAGAGAGACTGCGTGTCACGTTCGCGCTGGACTGCTGTGATCGTG
AGGCACTGCACTGGGCGGTGACTACCGGCGGCTTCAACAGTGAAACAGTACAGGACGTCATGCTGGG
AGCGGTGGAACGCCGCTTCGGCAACGATCTTCCGTCGTCTCCAGTGGAGTGGCTGACGGATAATGGTT
CATGCTACCGGGCTAATGAAACACGCCAGTTCGCCCGGATGTTGGGACTTGAACCGAAGAACACGGCG
GTGCGGAGTCCGGAGAGTAACGGAATAGCAGAGAGCTTCGTGAAAACGATAAAGCGTGACTACATCA
GTATCATGCCCAAACCAGACGGGTTAACGGCAGCAAAGAACCTTGCAGAGGCGTTCGAGCATTATAAC
GAATGGCATCCGCATAGTGCGCTGGGTTATCGCTCGCCACGGGAATATCTGCGGCAGCGGGCTTGTAA
TGGGTTAAGTGATAACAGATGTCTGGAAATATAGGGGCAAATCCAAMWWMRWMRMMMMWMW
RWWWWWMRMWMMMMRMMMMRMWMMMWWWTTGAGGTAGCCTGAGTTTAACGGACAC
TCCTTCCTGAAATMGMMWRRMMWMMRMMRRMRMYMMWMWWRMMRRRWMMMRMMM
KRWWWMWRRYRMWMMRRMRMMMRMRMMMAMMWRMMRRRMWMMWMMMMMMM
WMYRMWMRMRRWWMMMMRWMMWRMRMMWMMRMWWMMMRRRWMMWMMMRR
MRMMRWYWRGTCTGAAAACMTMMMWRTMRMRWMMMRGTAAMTGTTRMRAMMMMRMTC
MRMWRRMMMRMWWMWRRRRMMMRMWRMMMRMWRRWMMMRRRMMRRMRWMMM
MMRWMRWMRMRRTGAARGACCGTAATGGGTCCGGAGCAAAGGGCTTGCCCCATTGTGCTGATGG
TGCGGCGGCAACCACGGGCGACAATGCCGATGGGAGGACTGCTGTAAAATCGGCAAAGCCATTTCCT
GTAAGTAAACGACAGGTATGGGAAGCTTATAAACGAGTAAAAGCCAACCGTGGGGCGGCAGGCATTG
ACGGGCAGACACTGGCCGGGTTTGACGAGAATGTTACAGACAATCTCTACAAACTGTGGAACAGAATG
GCTTCCGGCAGTTATATGCCGCAGGCGGTAAGGCGTGTGGACATTCCCAAAGCTGACGGTGGTGTACG
TCCGCTCGGTATTCCGGCCGTGTCGGACCGGATCGCCCAGATGGTCGTTAAGCAGATACTCGAGCCGG
TACTGGAACCCCTGTTCCATGCAGACTCGTATGGTTATCGACCGGGTAAATCCGCACACCAGGCGATTG
CGCAGGCGAGGAAACGCTGCTGGAAGTTTGACTGGGTGGTGGAAGTCGATATTAAAGGCTTCTTTGAT
GATATCGATCATGACCTGCTACTGAAGACAGTGCAGCATCATACTCAGGCACGCTGGGTGGTGATGTA
CATTGAGCGCAGGCTGAAGGCACCGGTGCAAATGCCGGATGGAGCAATGCTGGCACGGGGTCGAGG
AACGCCGCAGGGCGGCGTAATCTCCCCCCTGCTCTCAAATCTGTTCCTGCACTATGCCTTTGATATGTGG
ATGCAACGTCAGTTTCCCGGCGTTCCCTTCGAAAGGTACGCCGATGATGTTGTATGCCACTGTCACAGC
CAGTGGCAGGCTGACGCTCTGATAAGTGGACTGAGGCAGCGGCTTGCGCAATGCGGGCTGCAGCTGC
ATCCGCAAAAAACACGAATAGTTTACTGTAAGGATGCTGACCGACGGGGGGACTACCCGGAAACGAG
CTTCGACTTTCTGGGCTATACATTCCGGCCGCGTCTGTCGATGAATCGCTGGGGTAAGACCTTCGTTAA
CTTCAGTCCGGCGATGAGTGCCAGAGCAGGAAAAGCAATCCGTCAGGAGGTGAGGAGAATCGCAGTG
ACAAGTCCTTGTACGAGCTGGCGCATATGTTCAATGCGAAAATCAGAGGCTGGATAAAGTACTACGGT
GCTTTTTATAAATCAGCACTGTATCTGACGCTGAGGCAAATCGATCGCAAACTCGTGTTATGGCTGCCCC
GTAAACACAAGCGACTTAGGGGCCACCGACGACGGGCCAGCCACTGGCTGGCCCGTGTTGCGCGGAG
TGAAACGAGGTTGTTTGCACACTGGCCGTTACTATGGGGACAGGCCTCAATGAGAAGAGCCGGATGA
GCTGAGAGGTTCACGTCCGGTTCCGTGAGCACCAGAGGGGGAGGTTCCCTCGGGTGACTCGACTTGTA
GTACATTGTTCACGAATGAGTGATGCAACAAGGCTGAAGAACAGGTTATCGGTGCGCTTCTCGGAAGT
CGGGTTGGTCCTGAACGCAGGGAAGACGAATATCGCCTACATTGACACGTTTAAAAGGAGAAACGTCG
CTACGAGTTTCTCCTTTCTCGGATATGACTTCAAAGTGCGTACTCTGAAGAATTTCAAAGGCGAATTGTA
CAGAAAATGTATGCCGGGTGCATCAAATGCAGCAATGTGCAAAATAACAGAAACAATCAAGAAGTGGC
GTATACATCGCTCAACAGCTGAGAGTTTGCTGGATTTTGCGAGGCGCTACAATGCGATAGTGAGAGGC
TGGATTGAGTACTATGGAAAGTTCTGGTCCAGAAACTTCAGCTATCGACTGTGGAGTGCAATGCAGTCA
CGTTTGCTTAAGTGGATGCAGTCTAAATACAGACTTTCGAACCGGAAGGCTCAGCGAAAGCTGGCGCT
GGTAAGGAAACAGTATCCGAAGCTATTTGCCCACTGGTATTTACTACGTGCATCGAATGAGTGATCAAG
AGCCGTATGACGCGAGAGTGTCACGTACGGTTCCGTGAGCAGCCGGAGAGTGAAACTCTCTCCGGCG
GGGTTCCGATAGCGGTGGCGAACATGCGGCGGTGTTGTACTCGCTGATCGGCACATGCCGTCTGAACA
ATGTGGAGCCAGAAAAATGGCTGCGTTACGTCATTGAGCATATCCAGGACTGACCGGCAAACCGGGTA
CGCGATCTGTTGCCCTGGAAAGTTGATCTGAGCTCTTAGTAAATATCAATACGGTTCTGACGAGCCGCT
TACGCAGATCATGCCATAATGAATAAACTTTAATCAGCGAAATTGACGAAGAAGCTGGCAGCGCTCCA
ACATMMRWWMMRMMRMMMRRWYMMMWWMMRMMMMMMMWRRMMRMMRMMRM
MMMRMMMMMWMMMRMRMMWRMMWYMMYRWMWMRMMMMYMWMMRMMRRGAC
TACTTTCACAAAGTCACAGCGACTGTCAGCAAAAAACACGTGATGATCGTCAATTACGTTATCACTGCC
AACTGACTTCACCTTTTGGTTCATATGAAGCAGCATCCAGCGGTGAATGTTCCCGGATATATTCGATAA
GTACTTCAGCATCAATAAATCCGGTATTTATGTATCCGGGTCTATTATCAATACGTGGATAGCCATCCCC
ACCAGTAGCATTAAAGCTAAATGTTGTCATACGATATTTTTTGTTAAGGTCAACCGATTTACCTTTTATTT
TTAGCATTTCGATTTTTCCTTTTTTGACTACAAAGCTGGTATTCAAAAATTGTGGGTAGGCTCCGGAATC
CGGTTTCATCTGTGCCACTGTAGCTAGGTAGTCAGCTACTTCTTTCCCCGTCAAATCAATACTTACTACC
GTATTACCAAATGGCTGAACTTTCAGTATATCTTTGTATGTGATGTTACCACTCTCTATGGAATCTCGAAT
TTCCCCACCACTGACAACAGCAAAATCTGCGTCAATACGTTCTGTCAGAGCGCTCAGAATCAGATGGCC
CATACTGGTCTGAACAAAACGAACTTTACTTCTGTCCCCTTCCAGACGTCCGTTCACAACCCCAACCTTC
ACATCCAGTTGTGCTTTGCCTTTATTCTGGAATGGTGTCAGTAAGGATAACATCTGTGGATCTTCAGTAA
TTTCGGGGGTGTAAAAAGAAAATTCCGTTTTTCCATCTTCTCTGGTAATTCTCATTTTGAGATTAACTGG
GTGTAACTGATAATTAACCAGTTTCATTGTACCGTTACAGAATTCAAAATCAGCCTGACCGACATATTTT
CCCCACTCATGCGCTTGTACAATCCAGATGCCATTTTGTTTATCTGGTACACAGGTTGTCCCCGGAATAT
AATTCCACTGTTTTTTATTATCGCTGGCCATGCAGACAGGAGCTTGTGAATGTCCTCCCACAATCATTGC
TAATGAGCCAGTCGGCAAACTGCGAGCCATTTCAACATCACCAGGAGCATTCGAACCTGACTCGCCATT
GTCATAGTGACCCATATGTGTTGCCGCAATAATAATATCTGGCTTTTCCTGCTGATTAAGTTCATCAATG
ACACTCCTCGCCTCAGCAGCAGGTTGACGGAACTCAATATCAGTAAAATATTCAGAATTTCCTGTTTTCG
CAGTATCATCTGTCGTCAAGCCAATGACTGCAATTTTCAAATTCTGACGGATGAAAATTTTCCATGGACT
GAATAAACGCCTCCCTGTACTTTTTTGATATATGTTAGCAGAAAGGAAAGGAAATGTGGCCCATTTTTCC
TGCTGACGTAATATATTTAGAGGATTATCAAATTCATGATTACCGACGGCCATTGCATCATAACCAATTA
GATTCATTCCACGAATATCGGGTTCAGCCTTCTGAAGGTCTGATTCAGGTACACCGGTGTTAATATCACC
ACCAGATAATAATAGAACACTTCCGCCATTATTTATTACTTTCTGCCGGATATTGTCCACCAGAGTTTTTT
GGGAGGATAACCCGTACTCACCATGATTGTTACGCCAAAAGTGTCCATGGTGGTCATTTGTATGCAGAA
CGGTTATTTTATATACTTTATCTGTTTCATACGCTATAGCAGGATTTCCTGTCAGTAATGACAGGGTGAA
CATTATTAGCGTAATGTTTTTTTTTAACGGTATCATTGTACCTGTATGAACGCATCCCCGTTTGCAAGGCT
TCCGTTGTTCTGACATCGACGAGTAGTTGCTGCTCTGTATCCGGCCTTATTGCAGTCAGAGCTGCGGGC
CACTATAAGATTCGCTGACCTGAGCCTTATTCCCTTCACGGACTTTATGTCTAGGACACAATTCAGGTTT
ATCAGRWMACGGTCTTACCTGTCGTTGTCATCACGTCGATCACCATAGCCCCCTTTCAGCATTCACCCT
TCTATAAGGTTTGATGTTTATCAGTTTCAGCAATTAATATGGTCTGATACTGACGTGAACCGCCCCAAGT
TTCCTGGAGAATGTTTTATCTGTGAACTCAGGCTGCCAGATCATCGTTTCCGATAGAAGCATAATAAGCT
TTTTCTGCTTCTGCCGGAGGGATGTGGCCCAGCCTTTCCAGCAATCGTCGATTATTATACCAGTCCACCC
ACGTGAGTGTGGCCAGTTCCACTTCTGCACGGTTTTTCCAGCTCTTACGGTGTATTACCTCCGCTTTGTA
AAGACCATTGATGCTCTCCGCCATCGCGTTGTCATACGAGTCGCCTGTACTTCCTGTTGATGCCAGTAAT
CCGGCTTCCTTAAGCCGCTGTGTGTAGGATACATACTGAGAACCTTTATCACTGTGATGGACCGTGCCG
GACGGTCGACGGGCCCATAACGCCTGCTCCAGTGCATCCAGCACGAATGTCGTTTCCATGGACGATGA
GACCCGCCACCCCACGATGTATCCGGCAAACACATCAATGATGAACGCCACATAGACGAAGCCCTGCC
ATGTGCTGACGTAAGTAAAATCAGCCACCCACAGCTGGTCAGGTCGTTCTGCCACGAACTGACGGTTTA
CGCGGTCGCCTGCGGCAACGGCTTTCCGGCTGATGGTCGTACGGACCTTTTTACCCCGGAGAACACCG
GCAAGTCCCATAACCGCCATGAGACGTGCCACAGTGCATCTGGCCACTCTGATACCTTCCCGTAACAAC
TRAMRMMMRMMWWWMMMMMSWMYMMMWKGTGATTTTCATCGTATACGCGCTGTATCTCT
TTCTTCAGCCAGTCATCGCGCTGCGCACGGGCACTGCGTTTATCCGGATGATGTCGCTGTTGCTGACAG
TGGTAATACGTTGACGGGGCAATATGCAGTTCGCTGCATAGCGGTCCGACCCCGTACTGCTCACGCAG
CTTATCCAGCAGTGGCATCATTTTTTCCAGAGGCGGTCAAACTCCGCCTTCGCAAAATAAGCGGAAGCC
TGGCGAAGGATATCGTTACTGCGGCGCAGTTCACGATTTTCACGCTCCAGCTCTTTCAGACGCTGACGT
TCAGCGGTGGTGAGCCCWCCATCACCGCCCCCGGTATCCCGCTCATGCTGGCGAACCCRGACACGCAG
AGTCTCCGGCGTACAGCCAATCTTTGGGGCAATGGAACAAATTGCCGCCCACTGTGAGTCATATTCATC
CTGACTTTCCAGAACCATACGAATCGCCCGCTGACGGACTTCGGGGGAAAAACGAGTATTTTTAGTCAT
CCTGTTTACCTCTTTCTCAGGRAGTTTAGTCTCCAGGATTCCCGGGGCAGTTCAGTTCAGGCTGTACGCT
TCCCCATGTAATTTGCCTCGCCACTTCACCGGGCTCAGGACCCCGTAGGATCTTCACCGAAAAAAATGC
ATCACCAGTTGCTTTATTATTTGTTTTAGGTAGACCTGTTACTAATTTATACCTACTCTGACCAAGTCTGG
ATAAGAAAGGCACACCTGCACATTTCTACTTGGAAATGTTTTCTTTTACCATTTTGCTCAAAAATTTCAAC
ATCCAATATTCCTCGTACAGAACTACTCAAGTCCTGAATAGAAAAAGTTCCAGCTGGTACAGTAGAGTC
ATATATTACTCGACCTTGTTGAGATACAACAACACGTGCATTAGTTTCTGCAATACCAGTAATCTGAGGC
GCATAACCACGAATATTTGGCGGTAGCATTCGTTCATCGCTTTCTAATGCAATTCCTGTGTAACGCCAAG
CAACAGCGCATTCTGGCGCATGATCTGGTAAACACGTTTGGCATTGATCGCAGGCATACCATCATGTGC
ACAAAAACCCACCGAGCTTTTACACGGTGGGTCCCAGAGAAATGCAGGACATCAACACGCCCTGCATTT
TTATTATCAGAAGGTATATTTCACACCCAAAATACCTTGGGTGTCTCTGTAACTGTTATTACCCAATTGCT
GAGATACGTTTCCCCAGATGCTTAGGTTATCTGCAAGACGCCCCCCCACACCAGTTTTGAGTTCAATCAC
ATTACGGTTGCCTATCTGGTGATTTGACATGCCATTCATAATTACACCATATTGCTTTGAGCTCCATTTCC
AATTCACTTCCAAAAATGGTTCGAACTTACGAAGAGTCTCCTTATCGATACAACTCTGACTAATTAAGGA
TGTACGCAATCCCAATTTTGCTTGAATATTATTGACCCCAGAGGATGTGACCATAGTGCCATTGTGTTCA
ATATAGTCGTTTGCTTTTACCCCATGCCTAGTTATCTGGACTTGTGGATTGAGATACAATGCGTTGTCAA
TATTATTATGAGCAGTCCAGCGTATGGTTGGGTAGATATAGCCAGCTTCCAAAGCTCCTGTTATTCCTTT
GCTGGAATATTTCTCACCAGTTAACCCATCTCCTTTGACTGTATTATTAAACCAACCGTACTGCAACCAA
GCATCCATATATAGACCTGTTCGTTCTTTTTCATTCTGAAACCAAGAACTGTACAAACCGGCAGTATAAC
CGCTCACAGTTCCTCGTGAACTATAACTAGTATACTTATTATGAGTTTTGCTATCCTGATATCCATAAGCT
CCCATAATACCCACATGCAAGCCATCATGATCAGTGAAGTTTGTCTTCAAAATATCTCCACCAATCTGAT
AAACCATCCTGTTAGCTGTGGTTTTTAATTGTCTATCAGCTAAATTATGCTCATTATGTCCACCAACAGTA
CGAATCCACATGGTGGTTTCGCTGAGCTGACCTGTAGTAGGATCAACAATAGTGCTACCGTGTCGGTCA
TAGAGAGAAGGACTAAACATCGTGTTCATTGCTATCAGGTTAGCAATATAACTGCCAGCTTCAGGGCGC
CATGTGTGAATACTACCTTCACCCAAATTAAGTGAAGATACAAGCTGTGAACTAGCTCGGCGACTACTC
ATTTGAGTAGACTCTTGATTGCTCATCTGTTTTGATTCTTGATTATCCATCTGACTAGTTAGATACCACTT
ATTGGTATTCAGTCCAGATACAGTGCCCTGTTTCAGGCGATAGTCATAACTACCAGCAACAATACGGCC
TTTCTGAATAAAAGCATTCTTATCAGAAGAATCTGTCGAAATAATTTGAACACCTTCAAGAGTTTTATCT
CCAGAGCCTCCCTCATTTGTCACTGCAACATAGGTAATACCAGACGCATGGCCTGCAATTTCCAGCCGG
TCAGTTTTACTGTTGTCATTGCCGAGAACCGCGCCCAAAGATAATATTCCGCCCTTTCCATGCCAATTAC
CTTTCTGAACATAGGTCTGACCGACATTTGACGAACTATTATTGAGAATAACATGACCGTTTTTCTCCAG
AGTCATGTTACCTGTGACAATGACGGGGTCAGGCAAAAAAGGGGCATTAATATTATTGATTAGCACAG
TCCCACTATTTAATAAAGTGTTAACAGTTTGGTTCATGCCTGAAAAATTCAAGGTAGCACCTTTATTAAC
AATAACACCAGCGGTACGTGTCATAGCTTCAACTGTCCCCATTTITTAGAATGCCTGCATTGATGTTGGTG
AAACCAGAGTAGGTATTTTTTTCCGCCAAAATGAGAGTTCCCTCCCCTGATTTTGTTAGGGATTTTCCGT
CCCAGCTATTTATATTATGATGGCTGAGATTATCTGCCAGAATAGTACTAACAGTAAGTTCAGCGTTTTC
TTTCAGATTGAACTCTCCATGACTGTCGCCATCTGTATCATTCCAGCTCAAACCGTAGATCAATTTATTCT
GGTCACTGATATAGCTTTTTGTATAGTCAATAGCACCAGTAGTGAGTAACGGATTGATGATAGATATAT
TAGAAACACTGAAACCTGTTATCCCTTTATCTGCACTTAGCAGTTTATAGTCCTTCTGCGCCCAGTTATTT
ATAATAGCTTCCACTTTTACATTGTTGCCAAATGACATATTTCCAGAAGCTTCCAGACTACTGATTCCAGC
TAAATTTAAAGAACTGTTCTCACCAAAAGAAACGTCACCTGAAACAGTGAATTTGTTACCTGATAGTGA
AACAGTTGTACAATCACCAGCACATAAATCACCTTCAATAGTTGGTGTGGTCCCAGTGTAATCATTGTTG
ATAATTTTCAGAATGGATGAATTGTTAACCTGTACATCACCAGTAATGACAGAACCTTCATGGAGTATTA
ATGAATTGTTTCCACCAGTGATGTGGATAGCATTACCTACTACATTTTGTGTTCCATCATCTTCTTTACCT
GAAATTATATATCCGCTATTAGCAATGGTCATATTGCTACCATTTATAGCATCCCCCMAATGTCCAMM
MWWRMMRMMWMMMMWMRCTMMAWWGTTGATTATAGACAGGTTATCTCCTGTGATAGCATT
ACCACCGTAGCCATCATAACCATAACCGTAAGCAGCACCTCCGTTGCCTCCAGCGAACGTGCCCTTATT
GATTACAGAGAGGCTGCTTCCTGTGATGGCATCACCTMMAWWACMGMMATMAMCMTMAMMA
WAMWWGWWAMMTMMMWWMMMAMMRRMMMWMRTMSMTTTGTTGATTATGGGTAGAW
TGAMTMMTGTGAMGGCATCACMTMMATTMMMMMMATCACTATCACTATAACTATCGCCACMM
WGTCCACCTGAAAWMMWTTCTCCATTTATTATAAACAGATTGTCTCCTGTAACAGCCTCACCACCATC
ACCGTTATGGTCAGCACCGCTACCGCCGTTACCACCAAGAATCATGCCTTGATTGATTATAGACAAGTC
AGATCCGGTAATAGAGTCACCACCATTACCGCCACAACTATTACCATCATTATTATCACCAGCACCACCA
TGACCTGCGCTAATCATAATATCAGAGTTAATCCGTAGCTTAGTTCTAGATGCATGAGAGGGGTTGGAA
GAAGTTATATACCACGCATCCATTCCATCTTCACCAGCTCCCAAGGGAGAAAGTCCATCAACAGGTGTT
AATAATTTTTCATAATTGTCCTCTGAAAAATGAAGTTCTTGAGTACCCGAAAGAGGAATAGCAAAAGCT
ATTGGCCCCCCGAGCAACAGGGATGCACCAACAACAAAAGGAGAAAAAGACAATTTGCATGTTTTTTC
CTTTACCGTAGGTAATTCTCCGGCCCCCCCCTGTGAACATTGGGTCATATTACAAAAAAATTTGTGAATT
TGATTCATGCACTATATTATCAGTAAGTGGTTGATAAACCCCTAAAAAGATTCCGTTTTATCCGGAATAA
AGGGACGATATATGCAAAAATATATTAAACAAAGCCTCAACCAAAACATGAGAGCTGTGTTATATATCA
ATGCAACGGGAGAAATTTACCTGTGTCTTGCTTTGAAAAAAAGAGGGGGAAAGCTATATGTTCGATAA
TAAATTAATAATTTTACTATGATAGTCTGCTTCAGACCGCAACTTTTCAGTTCTTTCTGTACGCTTGCCCA
AAGTTTAACCACTTTGTTTTTCTATACACCCAATGGTTCTATCTGCCCCTTGATGTTAATCCCCAAAGCCA
ATAACATGAATGTATTCATATCACAACTTTCCTACCAGACTCTCCAATGCAATAACTGCATAATTGTTTTT
AGTGGAAATGTATCGTTTTCTTAAAGAGAAGAATAACATTCCATTTTATTATGTTCCCAATTTAATTATAT
TAATATATATTGGCATTAATAGGAAAATACATCAGGAGAAATCAAATGCAGACATCAAACATAACTAAC
CATGAAAGAAATGACTCATCATGGATGTCAACTGTCAAATCAACTACAGAAGTAAGTTGGAATAAATTG
AGTTTTTGTGACGTTTTACTGAAAATAATAACTTTCGGCATATATTCTCCACACGAAACTCTAGCCGAAA
AATATAGCGAAAAAAAACTAATGGATAGTTTCTCACCATCACTATCACAAGATAAAATGGACGGAGAG
TTTGCCCACGCAAACATAGATGGCATTTCAATACGGCTATGCCTTAACAAAGGCATATGTTCGGTTTTTT
ATCTTGATGGAGATAAAATACAATCCACTCAACTTTCATCAAAAGAATATAATAACTTGTTAAGTTCACT
CCCCCCCAAACAATTCAACTTAGGAAAAGTACATACTATTACAGCTCCGGTATCAGGGAATTTTAAAACT
CACAAGCCTGCACCAGAAGTTATTGAAACAGCCATTAATTGCTGTACATCGATAATACCAAACGATGAT
TACTTCCCCGTTAAAGACACTGATTTTAATAGTGTTTGGCATGATATTTACAGAGATATTAGAGCATCTA
ACAGCAATAGTACAAAAATATATTTCAACAATATTGAAATACCACTAAAATTAATTGCAGATCTAATTAA
TGAGTTAGGAATTAATGAATTCATTGATAGCAAAAAAGAGCTTCAAATGCTAAGTTACAACCAAGTAAA
TAAAATAATCAATTCAAACTTCCCCCAACAAGACCTATGTTTTCAAACAGAAAAACTTTTGTTTACTTCAC
TCTTTCAAGACCCTGCATTTATATCTGCACTAACATCTGCATTCTGGCAATCTCTTCACATCACGTCTTCCT
CTGTGGAACACATATATGCCCAAATTATGTCCGAAAACATAGAAAACAGGCTTAATTTCATGCCTGAAC
AACGAGTTATTAACAATTGTGGACATATTATAAAAATCAACGCAGTTGTCCCTAAAAACGACACTGCAA
TCTCTGCCTCTGGGGGGAGGGCTTACGAAGTTAGCTCATCAATATTACCATCGCACATAACATGTAATG
GTGTTGGTATTAATAAAATAGAAACAAGCTATCTCGTTCATGCTGGAACACTTCCAAGCAGTGAAGGGC
TTCGCAATGCTATCCCTCCAGAAAGTCGTCAAGTATCATTTGCCATCATATCTCCTGATGTTTAAGCCAAT
ATTATATTGGCTGCCAGACTGTAAAAAAACGTTCTTCAGAACAGCTCTATAAACTATTAAACCAAAGCTA
AAAGGATGGGGACCAATAGAATCTCAGCCCATCCTTTTTATATCATTATGAACCGCCCCGGAAATCCTG
GAGACTAAACTCCCTGAGAAAGAGGTAAACAGGATGACTAAAAATACACGTTTTTCCCCTGAAGTCCGT
CAACGGGCAGTTCGTATGGTTCTGGAAAGTCAGGGCGAATATGACTCACAATGGGCAGCGATTTGTTC
CATTGCCCCAAAGATTGGCTGTATACTGGAGACTCTGCGTGTCTGGGTACGCCAGCATGAGCGGGATA
CCGGGGGCGGTGAGGTGGGCTCACCACCGCTGAACGTCAGCGTCTGAAAGAGCCGGAACGTGAAAAT
CGTGAACTGCGCCGCAGTAACAATATCCTTCGCCTGGCTTCCGCTTATTTTGCAAAGGCGGAGTTCGAC
CGCCTCTGGAAAAAATAATGCCACTGCTGGATAAGCTGCGTGAGCAGTACGGGGTCGGAYCGGTATGC
AGTGAACTGCATATTGCCCCGTCAACGTATTACCACTGTCAGCAACAGCGACATCATCATGATAAACGC
AGTGCCCGTGCGCAGCGCGATGACTGGCTGAAGAAAGAGATACTGCGCGTATACGATGAGAATCATC
AGGTATACGCTGTGCGTAAAGTCTGGCATCAGTTGTTACGGGAAGGTATCAGAGTGGCCAGATGCACT
GTGGCACGTCTCATGGCGGTTATGGGGCTTGCCGGTGTTCTCCGGGGTAAAAAGGTCCACACTACCGT
CAGCCGGAAAGCCGTTGCCGCAGGCGACCGCGTAAACCGTCATCAAGGCAATATGCCGCGTACTCCGG
GTGGCCCGCAGCGGCTGGTATACGTGGTGTCAGCGGCGGACAAGGATAAACACACGTCAGCAGTTCC
GTCAGCACTGCGACAGCGTTGTCCGCAAGGCTTTTACCCAGTCCAGCGTTACGGTGCCCCACGCCTGAC
GGATGAACTGTGTGCTCTGGTTACCACTTTAACGTAAAAACCGTGGCAGCCAGTCTGCGCCGTCAGGA
ACTGAGCGCAAAAGCCTCCCAGAAGTTCAGCCCGATCAGTTACCGTGCACACGGCCTGCCTGTATCAGA
AAATCTGCTGACGCAGGATTTTTACGCCAGTGGCCCGAACCAGAAGTGGGCGGGAGACATCACGTACT
ATTACAGCTCACCGACTGCGGGGAAACACGGGGCGCCGGGTTATTGAAGGTGACCTGTCCAGCTACTT
CGACACAGTACATCACCGGCTGCTGATGAAAGCCGTCTGCCGCAGGATCAGTGATGCACGTTTTATGA
GACTGCTGTGGAAAACGCCGTGTTGATGTTGGTCTCTTCCGGGCGGTCAGTGAAAGTATGCCACAGGG
CGGTGTGATCTCGCCGTTGCTGTCGAACATCATACTGAATGAGTTCGACCAGTATCTGAATAAACGCTA
CCTGAGTGGAAAAGCCAGAAAGGATCGCTGGTACTGGAATCACAGTATCCAGCGAGGCCGAAGTACT
GCTGTCAAAGAAAACTGGCAATGGAAACCTGCCGTTGCTTACTGTTGCTATGCCGATGACTGTGTGCCA
CGAAGGCGCGTCCTGGGGACGTGAGTCCCTGTGCAGCCATGCGGCACAAGAGATGAGGGAAGGCCCC
TCGGTGTCGGCGTGCAGGCGCAGGCACCAAACCATCCACTGCTGCTGCATTTAAGAGATGTGGTGGTG
AGCGAGATGGGAAAAGGTCGGACGTGAGCCGATCAGGTATGGTTCGAAGAGACGAACGAMAGTGAA
CCGCTGATGATGTRWMRAMAGMMWMWMRATGATGTCAMAACAATGGTGGAGTCGTTCCTGTGG
GATATGTTTACTGCCTGCGCGGCATCCGGCATAAAAGCGGCGTGAAGTCGATTCAGGCTCTTGTGGGG
AACGTGGACCTGTCACCCCGATGTGAAGGGAGAAGCGCAAGTGGAAGCCCCATGAGCGCCAGAGTAC
CAATGCGGGGTACAGGGACGGAGCACCAAGCGATTGCGCAGTAAACATGCTTGACAACTTAATTGGG
GCCCCCTCTTCCTTTTATGGTGTAATTGTTTGATTGAATACTTCCGGGGATTTCAGTATGAAATGAAGTA
TATTTAATATACTTTACAAAAAGTTTGTATTAGTGGAATAAGGACAATAGTTTATCCAAACTGCCAAGCC
CCTTTATTATAAGTAAGATCTGGTACTAAATAAGCGTTCTAAACCGCCCCGAGTAGGCGGCCTGACGGC
TGCGGCTTGTCATGGCCTGTGATTACCGTTATAAAAACAGACAATATCATTGTCTTTCAGGTAGTTATAT
GGCCCGTTCAGCTAAACCACGCAAACGAAAACCTGCACCTCAAAGAAGCAAACTCCTCCGCTATGTCGT
AAAACTTCACGAAGATGATTTTTTTGACGAAGAAGAAGCTGAGGTTCTGCGCTTTGATAATTTTGACGA
TGCCGTTGAGTGTTGCGCAGACCTGAATATTCCGTTCTTTGTGGATGCCGGAAACAAAAAACTGGTCTT
CTGGTTTGTACGTGTTGATGACGAAGGGTATCCGGAAATTGCCCGCTGCACGGAGCGGGAATTTGCGA
CAATTCCTGCCGGTATCAGCGCCGACGGCATGTATTGCCCTGAATGCGGTACGGTTCACTGGCCGGATG
GCGTTATTCCGCCTTTCTGATGCTTCCCCGTTTTGCCGATATTTTTCAGCAGGGAAACCGATGGCTTAAC
TGGCTGGAGAAGCAGCCGGTGCAGATGAGCCGCCTGGAACATTATGCCGGTCAGGATGAAATTGGCC
TGCGTTACAACAGCCACCGGACAAAACGAGAAGAAAACCTTGTGATGAGTGGCGACGAGTTTATGGAA
AGGTTCTCCTGGCATGTGGCGGATAAGGGGTTCCGGATGGTGATACGGGGTCCTGAGTCCGGTGAAG
CGGCGATTACTGGAAGATGTGGTGTACGCCATAACGGAGACAGTGAGAAAAACGGCGAGGCAAATCA
CAAGGAGAGGGATGTATCAGCAGTGACTGAAGGTTGACCCGCTGAAGTGCGAGCTGTGCGGAGGTCA
GATGCGGTTTACGGGGCTGAAGAGGGGTTACCGTCTGGCAGAGCAGGTTATGATGCATGAGCCGCTG
GCTCGAATACGGTGGTGCGGCTGAGAGTCGCAGAGGGGAAGTTGCGTCCATGTTKMRRRRMMMRR
MRMMMMMMMMMMWMAGAGATACATTGAATCAATCAATGTAAGCAATATTAAGTGTCTATACC
GATTACTTGATGATCAGGTTGTGTTGAATAAACGCGCATTCTTCGGTGCTTTTTTAATCTTTTGGGGATT
TAAATTCCTATCGATGGACATGAATGCAGGCTATATAAGAGCAGCGCGCATCCACTTACCCAATGCGGT
TGAGAAAATAGCCTTCGATCGCTTTCATGTGGCGAAACAACCGGGTGAGGTGGTGGATAAAACCCGCC
AGAATGAACCGCCCCGGGTTTCCTGGAGAGTGTTTTATCTGTGAACTCAGGCTGCCAGATCATCGTTTC
CGATGGAAGCATAATAAGCTTTTTCTGCTTCTGCCGGAGGAGTATGGCCCAGCCTTTCCAGCAATCGTC
GATTGTTATACCAGTCCACCCACGTGAGTGTGGCCAGTTCCACTTCTGCACGGTTTTTCCAGCTCTTACG
GTGTATTACCTCCGCTTTGTAAAGACCATTGATGMTCTCYGCCATCGCGTTGTCATACGAGTCGCCTGTA
CTTCCTGTTGATGCCAGTAATCCGGCTTCCTTAAGCCGCTGTGTGTAGGCCAGCGATACATACTGAGAA
CCTTTATCACTGTGATGGACCGTGCCGGACGGTCGACGGGCCCATAACGCCTGCTCCAGTGCATCCAGC
ACGAATGTCGTTTCCATGGACGATGAGACCCGCCACCCCACGATGTATCCGGCAAACACATCAATGATG
AACGCCAMMTMRMMRMMGMMMWRMMWTGTGTCAACGCCACGATGTTTGACCGTTATTTGCCA
TTTTCATGCCCGCTAAATACTTTCGTTACTGATGCTGTCCGCTTTCCACCATTTCATCATCACTGATTGGC
GTTGTGTTTTTGGTCAGCACTCCAGCTTTACGTTTCTCTTTTAACCGGTAACTCTCTCCTTTGATATTCAGC
GTGGTTGAGTGATGTAGCAGCCGATCCAGGATCGCTGTTGCCAGTACGTGATCTCCGAACATTTCTCCC
CAGTCTGCGAACCCTTTGTTTGACGTCAGTATGATGCTCGCTTTTTCATATCGACGGTTCAGTAGCCGGA
AGAACAGACTGGCTTCCTCTCTGTTCATCGGCAGATAGCCTATTTCATCCAGGATCAACACCCGGGCAT
AACTCAGTTGCTGTAGCTGACGCTCCAGCCGATTTTCCTGTTTCGCTTTCATCAGTGTCGCGATCAGTCT
GTCCAGTGGCATAAACAGTACCCGATGTCCCGCATCCACCGCTTTCACGCCAAGAGCTATGGCCAGATG
AGTTTTTCCGACACCAGGAGGGCCCAGCAGGATCACGTTTTCGCTGCGCTCCACGAACGCCAGACCAG
CCAGTTCCCGGACAACCTTACGGTCGATGCCCGGCTGGAAGGTAAAGTCGAACTGCTCCAGCGTTTTGA
CCCACGGCAGACGAGCCTGCTTCAGCCTGGACTCCATACCGCGCTGATGCCTGCCGTTCCATTCCTGCT
GTAGCGCCATGCACAGGAACTCCCGGTAGTTCAGCTCTTTTTTAGCTGCCTGTTCCAGCAGGCTTTCAAC
GTGATAACTCAGATGCTCCATTTTCAGGCGACTCAGCAGGACTTCCAGTTCATGCATCACAACAGCTCCT
CATAGGCACTCAGTGGTCGATGTTCCACCTGACTGACCTGCTGCCAGAGCGGGGCGTGATGCTCCGGC
ACTGTCTGCCAGCCAGACGATGCTGAACAGAGGCGATGTGAGGCCACCAGTTTCTCATTACTGTAGATC
CGCAACTCGTCATCCAGCGATATTCGTATCGATACCGGCTGACCACACAGCGCTTCGGGAACGCTGTAA
CGATTACCACCAACCTCGATATAGCTGTCCCAGGACACATGGCGGATATCGAAGTAACTGGTATCGAA
GTCTGTATCCGGTAACGGCTGCAGATGTTCCTGCTCCAGCGCGAAGCGCTGTTCCGGCGTTTCTTTGAA
CTGGCGAAGCTCCCGTTTGTCAGCCACATCGGCTATCCATTGCTCCAGTTGTTGATTGACATGAGTGAA
GCTGTCGAACCTGCGGTACCGGACGAAGAAGTTCTCCTTGAGGTATTTCACCATCCGCTCAACCTTACCT
TTTGTTCTGGCCCTGCGTGGACGGCATGCCCGTGGCAGGAAGTTATAGTGGTCAGCCAGCAACAGGAA
TCCGGAGTTGAACACGACTTTCCCGTTGTTATTCTTCAGCACCGCAGCCTTCTGGTTATCAACCAGCACC
GTTTTCACACAACCACCGAAGTAGCGGAAGGCGCGAACCAGTGATTCGTAGGTATGCTCAGCATCCTG
TTTTGGTGCGGCGAAGACATGGAAGCTGCGGGAGAACCCCAGCGTATTAACCGCAAAGTTAACTTTGC
ACCGTTGCCCGGCAACCTCCACCTCAACTTCGCCCCAGTCATGCTGGAGCTGGTATCCAGGCTGAGTTT
CGAAGCGAACTGTTCTTTTTGACGGACGCATTTTACGTTTGGGCTGGATGTAGTAACGCAGCATGGAAC
GTCCGCCCGTATAACCCATTGCCTTAATCTCCGCGAAGATAACCTCGCTATTCCAGACATTCTCTGCCAG
GCGCATGTCGATGTAATCCATAAACGGTTTCAGCTTAACCATTTTGTGGCGGGTCTTTCTGGCTGGCGG
TTCAGGGTATTTGAGGTAGCGTCTGACCGTCCGTTCAGAGCAACCAATCTGAGTCGCAATATCGACGAT
GTACGCGCCCTGCTGGCGCATTTGCTTTATCATGTAAAAGTCCTCTCTGCTCAGCATGTTGATGTCCTTT
CTGGTGTGAGAACCTTAAGGAAACAACATGTTGGGTGGAGCGGACAATCCAAATGGCGAATTACCGTC
TTATATCACTGGCGCTGACACATGTGCTGACGTAAGTAAAATCAGCCACCCACAGCTGGTCAGGTCGTT
CTGCCACGAACTGACGGTTTACGCGGTCGCCTGCGGCAAAGGCTTTCCGGCTGATGGTCGTACGGACC
TTTTTACCCCGGAGAACACCGGCAAGTCCCATAACCGCCATGAGACGTGCCACAGTGCATCTGGCCACT
CTGATACCTTCCCGTAACAACTGACGCCAGACTTTACGCACACCGTATACCTTGTGATTITCATCGTATA
CGCGCTGTATCTCTTTCTTCAGCCAGTCATCGCGCTGCGCACGGGCACTGCGTTTATCCGGATGATGTC
GCTGTTGCTGACAGTGGTAATACGTTGACGGGGCAATATGCAGTTCGCTGCATAGCGGTCCAACCCCG
TACTGCTCACGCAGCTTATCCAGCAGTGGCATCATTTTTTCCAGAGGCGGTCGAACTCCGCCTTCGCAAA
ATAAGCGGAAGCCTGGCGAAGGATATCGTTACTGCGGCGCAGTTCACGATTTTCACGCTCCAGCTCTTT
CAGACGCTGACGTTCAGCGGTGGTGAGCCCTCCATCACCGCCCCCGGTATCCCGCTCATGCTGGCGAAC
CCGGACACGCAGAGTCTCCGGCGTACAGCCAATCTTTGGAGCAATGGAACAAATTGTCGCCCATTGTG
AGTCATATTCGCCCTGACTTTCCAGAACCATACGGACTGCCCGTTGACGGACTTCGGGGGAAAAACGA
GTATTTTTAGTCATCCTGTTTACCTCTTTCTCAGGAAGTTTAGTCTCCAGGATTCCCGGGGCGGTTCAGA
ATGAACATCCGCATCTTCCTGTTGAAAGTCGTCGTCAGGCAAAGGGAACTCGCTTCCTGTGGCAGCACA
GCGATAAATGGATGACCGAATCCAGGCAGGAAAAGCTGATATGGCTGCGTGCGCAGATGAAGCTGAC
GAGCCTGTGCTGGGCGCTGAAAGAACTGGCAAAGGATATCTGGAGCAGACCATGGAGCGAGGAAAG
ACGCAACGACTGGCAGAGATGGCTGTCGCTGGCGGCCAACAGTGACGTCCCCATGATGAAGAATGTG
GCTAAAACGATAGGGAAAAGGTTGTACGGTATCCTGAACGCCATGCGCCATGGCGTATCAAACGGAAA
TGCGGAAGCACTGAACAGCAAGATCAGGCTGCTGAGAATAAAAGCCAAGGGCTACCGCAACCGGGAG
CGCTTTAAGCTGGGAGTGATGTTCCACTATGGGAAACTGAATATGGCGTTCTAAGCCTTCCCACCATGA
TCGGGGAAGACCCAGATATGAACAGGCACATTCAGAGTTACTTTAAAGAGCTTACTGAACGTCTGAGG
TGTTAGAGCCATGACTGCCGCAGCATTGCTGGCGGAAATGCCTGAATCGAGCAGCCTCTCGAGACGTG
AAATCAGCGCACTTGTCGGTGTTGCTCAGGTAAACCGGGACTCGGGAACTCTGCGCGGTCGACGAACC
ATTTTCGGTGATTGTGCTGGGGAAGAACAGCTCTGTACATGGCGGCGCTTGAGGCACCCCGGTTTAAT
CTGGTGATAAAGGCGTTTTACATGCGCCTACTTGCAGCCGGAAACGCCAAAAAAGTGGCACTGGTTGC
CTGTATGCGTAAGTTGCTGACCATTATGAACGCAATGCTCAGAAAAAACGAAGAATGGAATGAATCGT
ATTTATGAAAACTGGATTGTGGGCCATAAATATTCCAAAAAGATAGCCCGCTCCGTTTATTACCGGAAC
GGGCGAGAGCATGACATTGTCTGTAATTATAATGAGACTGCTGATTCTACTTATTTTTAATTTCAAGAAA
AGCGTTTTCTACAAAGAATTATGAGATTTAATGTTTTGTTTAGACAATATTATCATTCTGTTCAATAATGA
AAGTCTTCATCAACAACAAGTAAAAACAACGTCCCTCTCATCGAACCGGACTTGCAGATTTCCCGCATCC
GGCTCTCTTGCAACACATCAGACCTTCACGTTCGGGCGGTTATGGGGAAGGTCCAGCAAGCTGTATGA
GCCCTAACTGTTTGAATACCAGTAGATTACCAGAAACTTCTATCGATTGAAGACATTACCAACTAGCGTT
ACTAAATGTTGACCAAGTTTTGTTCACTTGTCTGGTTTTGATAGTAGCATTCTGCAGAGTATTTTGATAT
GATAAATATGCATTAACAATGATATTATTGTTAATTTCCTAGCATGAAAATTAAAAGATCAACTTTTTATC
TCGAATATTTTTTACATTATCAGTTGGTTTCTTATGAATGACAATTCATTGCTTAGAAATTCTTCATTATTC
ATCGCCTATATGGGATGTGTAGGGTGGGTCAGTGCTTATTCTTATGGCTGGGGAACCTCTTTTTACTAT
GGATTTCCATGGTGGGTTGTTGGTGCAGGTTTGGATGATGTTGCCAGAAGTTTACTCTATGCTATCATA
GTAATGGGTATACTTTTTACCGGTTGGGGGATTGGTATTTTATTTTTTCTTCTTATAAAGAAAAGAAGTA
AAATACAAGACTTGAGTTTTTTTAGACTATTTTTTGCTATTACCCTACTGTTTTTCCCTGTAATATTCGAAC
TTTTAATTCTCAAACAATATTTCATACTTCCATTATCACTTTCATGCATTATTAGTTCATTGGTTATTTCAA
TAATAATTCGTATATATGGACGGATCTTTTCTGTATCATGTTTTTCAGATATCCCTTTTGTTAGAGAACAC
CGCATTAAGTTAATTATGGCTGGTTTTTTGGTTTATTTTTGGCTTTTTTCCTTTCTTGTTGGCTGGTATAAA
CCTCAATTAAAAAAAGAATATCAGATGCTTTGTTATAATAATAGTTGGTATTATATTCTTGCTCGTTATG
ATTCACGGCTCGTTTTGTCTAGTTCATTTAAAGATGACAGTAACAGATTCTTAATATTTAATACAGAACA
GTCGGGATTTTATGAAATCAATGATGTTTATGTCAGAAAGTAATAATCCTCTCCCACAAAGTGAATTAA
ATGAATATATAATGCGTTCGAAGTATATGTATTGAGCTGTCATACTGAGAAACAAACCTTAGGGCAGCA
CAATTAAAGCCAGAAACAACTATGCAAGAAAGTTACTAAATAAGTGCTCGGAACAGTAAAACACTCAG
GGGGGACTCAGTCCGGATTATGTAATCAGATAAATTGCCAAACATCACAAATCACCAGCCGGACTGAG
TGATGCCGAGCATTGCACCAATACCCCGTGATGAACGATGCCTGATGCAGAAAGCTCCCTATAAAACAA
AGAACAAAAACTACGACCGGCGATTCACCGTCATACTGATGCTGCATCGTGGTGACACTGTCTGTCACT
TTCATGTTGACTGAACAACTCTGCTGCGAAGAAGGTGTACCGCCGGCGGGATTTGATTGTCCGGCGTCT
AGCTTCCACTTCCCTGATGGAACTGAAAGTTGTTATGGCCGGTCAGCTTGCCCTGTCCGCAGGACATGA
GGTGCTGTGACGGACAGCACCAGTAACGGAAAGTGACAATGAAAATGAATACTGCGCTGATAGTTGC
ACTAATGTGTATGTGGTATGCGGTACCCGCTGCTGCAAAAGAGACTTTACTGGCTATGCCACGTAATTC
AACAGAACATTGCTATGCGGAGATTAATGTGCACGGACCATATGGGGTGTATTTCCGAGTGGTCCCGC
ATCCCCCCGGGGGAAAGAGCTGGGTCGAATGTAACAGTGACTACTATTACTCTGATAAACCACCGGGA
GTACAGATACTCGGAACCCGTGCAGGGTGTCGGGTATACGGAATCTGCGGGACCACCTCGACACTGCA
TGTGGCTGGAAGAGGGGTTGTGTGTATTAAAAATATATGCAGTCCACGTGGGATGATAATTCACAGGA
TACGGAAACGTCCTGTAGTGGCTGTAAGCGATGAGATGTGATCTTACCCAGCAATAGTGGACACGCGG
CTAAGTGAGTAAACTCTCAGTCAGAGGTGACTCACATGACAAAAACAGTATCAACCAGCAAAAAAACA
CGTAAACAGCATTCGCCTGAATTTTGCAGTGAAGCCCTGAAGCTTGCTGAACGCATCGGTGTTGCTGCC
GCAGCCCGTGAACTCAGCCTGTATGAATCTCAGCTCTATGCGTGGCGCAGCAAACTGCAGCAACAGAT
GACCTCGTCTGAGCGTGAAAGTGAACTGGCCGCTGAAAATGCCCGCCTGAAGCGGCAACTGGCGGAG
CAGGCAGAGGAACTGGCTATCCTCCAAAAGGCCGCGACATACTTTGCGAAGCGCCTGAAATGAAGTAT
GTCTTTATTGAAAAACATCAGGCTGAGTTCAGCATCAAGGCAATGTGCCGCGTGCTCCGGGTGGCCCG
CAGCGGCTGGTATACGTGGTGTCAGCGGCGGACAGGGATAAGCCCTCGTCAGCAGTTCCGTCAGCACT
GCGACAGCGTTGTCCTCGCGGCGGCCTTTACCCGGTCAAAACAGCGTTACGGTGCACCACGCCTGACG
GATGAACTGCGTGCTCAGGGTTACCACTTTAACGTAAAAACCGTGGCGGCAAGCCTGCGCCGTCAGGG
GCTGAGGGCAAAGGCCTCCCGGAAGTTCAGCCCGGTCAGTTACCGCGCACATGGCCTGAGGTGTACTG
GCAATAGCGGACACCACCATTTGTTCTTTTTCTAAGCAGTCATCTGATAATATTTTATCCTGAAGGCTGC
CGGGGAGATATTCCCCAGACGAGAGTGACGACGCTGACGATTGTAGAAAATCTCAATGTATTCCCGTA
TTACTGAGATGGCCTCATCCCGGCTCTTAAAACGATAGTGGCTCAGGCTTTCATTTTTCAGCGTTCCCCA
GAAGCTTTCCATCGGAGCGTTGTCGTAACAGTTTGAGGTAGCMWRMSWKMRWMWWMMRMWM
YMWWMRMWMMSRRMWWRRMMWYRMMRWWMRMWMMKMRMWWMRMMWMMMWW
MRMWMMMMMRRRMMMYWMMMWWKWWMWRMMRMWWWWWRMMWMRWWWWW
WYRMWRMYWWMMMMWRWYRRRRRMWWWWWMSWMMRRRMMWMWSMMWRMMRR
MMMMMWMWRWRWRWRWWMWRWMMYMRWMWRWMMRRRMWRRMMWMWRMWRW
WRWWRRRWWMWMMMWMMRMMMMWRMMMWWWMWMWMMMRRRMMMWRRMW
WRSYMSWMWMRYRMWKMWWWRWMMRYMMMRMWMMWMWRWRMWMWMMMRMM
RRWMMWWMMMMWRRRMRWWMMWWMMMWRWKMMMRMMMMMRMWYRWMMMR
KMYWMRMRMMWKWMWWWWWWRMMMMYRWWWMMRMYMWWWMWRMMMMMWR
MYMWMMMMMMRWMMMWRMRMMMMMWRWWMMMRWMMWMRMMMRRWYMWM
MMRWMMMSRMSRMWRWMWWWMWRKMKRMRWRMMWRKWMRMMMMRMRMMWWR
RMWMWMMMRMWMWWMMMRMMWMMWRWMRRMMMRRWWMWMMRWMRRWWW
WWMYYRYRRMWWWWRWMRWRRYMRWWMMRMMRKWMMMWMMWMWRWWMMRMM
SRMMRMMMWMWWRMRRMMSRRWRMMRMMRSMMWYMMRKWRWWRMMWRMWMWW
WMWRRMMRRMMWWWYWWMWMMMMMRKRMMMMMMRRMWRWWWKMKMWRMW
MMRMMRMWMRWMMWMMMMMMRMMRWSWMMRMMYWWMMWMRMMMRMMMRW
WMRWMRYMRRWMWRMRRMKWWWMMSRWWRMSRWRWMMRRMYMMMRRMMMYMRS
MRRRRRWWKMMRWWRRRKMMRWWWRWWWMRMMRRWWMWMRMWMMYMYRRYWRW
WMMWWMWRWMRRWRRWMRYMRMMMRRMMMMMRMRRRWWWWMRYRMWMWRMM
RMMWMWRMMRRMWRWWRWWMRMMMWRRMRKRMWMMSRRRMRMWYMMMRWWM
WMMMRRWRMWRMMRMMSMMSMWWRWYRMWMMMRMKWMWSWMMRRRRYRWWMW
MRWRMMRRRMMMYRWMMMMRRMMWKWWMMMRMMKWWMMRRRMMRWMYWWWW
KMYRMMRWRMMMRMMRMMMMMRRMMWRMMMRRYMMRMMMRMYMMRRWWRRRRW
WWRRYWWYMMRMMWRRMWWMMMMMMMMMRRMMWRWRRMMRRMYYYSKMMKRYW
WMMMWRSWMRMWMMRRMRWWMMRRKRWMMMWRWMMMMWRMMSMMRRWRRMR
WKMKRRMMWRWRMMRMRRMWWRRWWRWRWRWWWMWMWWMMWRWRWRMWRMS
MRRMYRWWRMRMYMMMMMSYRYMSMWKKWRSYMRMRMWRWMMYMMMRMMWRMMM
MMMMWKMMRWRMMRRMSRMWRMMMRMMSRRWWMRWRWRRMMMRRMRWMMMMR
WRWMRRRMMWMKWMMMMRWRRWMRWMMSSSMMMMMMWWMRSRRMWWWRWMW
WMMKWRWMYWMMWRMRRYWMMMRMRRMRRMRRMRMRMRYKMWSRWRMMWMMRR
MMWRYRSMMRMRWWMWRMRMRRRWWWRMWWMMSRWMWMYMYWMRWWRRWMRR
WWMWWMRWRWMMRWMMRMMMMYMMMRMMRRMRYSMMWWMMRMRMMMWRMR
MRWRWRRMMRRWWWSMWYMMRRWKSMKRRMMMSMMRMMMWMRMWSRMMRMWRM
RRRMRMMRWWWKMKRMWWWWRRWWWMWMWWWMRRYWWRMRMRRRMRRRSMRRR
MWMRWMMMMRWRMMMWMRWRMWMMRMMMRMRMMKRMWMMMWRYMRWWRM
WRMMMKRMYMRWMRMRMWMSRMWWWYWWMRMWRMRMWWMWRMRKWWMKMRKR
RMWRMRMMSMWWMRRWMMRMMKMMMMRKMMRRWMWRRMMMMRRMWRKRRRMM
WWWMMMMMWRRMMWYMWRWMMYRMMMWWMKMMMRMYWMRMMMWMRRWMW
MRWYRRMYRMMMMYMWYMMMRMMMMRMMRMWWKMMMSRMRRMRWMWMWMWM
MRRWRWYMRRMWWMMWMMMMRRRMMMRWMRRMMMMMMRMMRWMRMWMMMR
MRMKWWKMRMMMRMMMRMKRMWMMWRYMSMMWRRMRMRRMRWMRMRWWWWW
WWMMRMWWMMRRWMRMMMMRRYMRMMRWRYRRRWMWMMMMWMRMWWWRWMR
MMMRMMWRWRMMWWKWMYWMMMWMMMMMMMMSWKRRYRMRWMMMRMMMRR
WWKYYWMWWWWYYRKMWMRMMRWMSMKMMWRKMMRRMMKWMMMMRMWMWRM
MRMMRMRRWRWMWWWRRKMRRRMMMMWRMRWWWMWMMMRMRWRWMMWRRRM
WMWMWMMRMMRRWMMYRRMMMYRMSMRMRMMRMRKMWWWKMMRYRRRMMMRM
MMMWMMMMMMSRRWWRMRRMRMWRMWRWRWMMMKWMMRWMMRMWMMMMMR
RWWMSWMMRMWWMWWMWMRMMMWWRWRMWRMMRKMWMWWMRWMMMMRWY
WMMMWMRWRMWMMWRRMWWWWMWMWWWWWRKMMMWWRMMRWMWMMMM
WRRWWYMMMMMMRRMMMWRRMRMMWRWRRWRMMMWRMRKWMMWWMMMRRMR
YRMMWRRMRMMWWMMMRWMMMYRMWMRRMMRWMWRMMMMMWMRWRMWMRM
WWRWSWYRMMMMMMWRYRMWMRRMRMWRKMWRMMMMMWMRWKMWMRMWWSKR
MWRMMRMMMKRSRMWMSMWWMMRMWRMMMWWWWRMMSMCTGAGTTTAACGGACAC
TCCTTCCTGAAATAGAATGGCATCAGAAGGAGCCAATAATGAGCAGAAAAACCCAACGTTACTCTACG
GAGTTTAAAGCCGAAGCCGTCAAAACGGTTCCTGAAAATCAACTTTCGATCAGTGAAGGCGCTTCCCGA
TTATCTGTTCCTGAAGGCACTTTAGGACAATGGGTTACCGCCGCAAGAAAAGGGCTCAATACACCAGG
CTCCCGCACAGTGGCTGAGCTGGAATCTGAAGTTATGCAACTGCGTAAGGCTTTAAATGAAGCACGAC
TTGAGCGAGATATATTAAAAAAAGCAACAGCGTATTTTGCMCAGGAGTCGCTGAAAAATACGCGTTAA
TCGAACAATGGCGACAACAATTTCCCATTGAAGCGATGTGTCAGGTATTTGGTGTATCCAGGAGCGGTT
ATWMCGACCGGGTACMRCMTGCCCCCTCAGACAGAAAACAAAGTGATGAGCGGCTAAAACTGGAG
ATTAAGGTGGCACATATCCGCACTCGCGAAACATATGGAACCCGGCGGCTCCAGACGGAGCTGGCAGA
TAACGGAATTATCGTCGGTCGTGACAGACTGGCACGTCTTCGTAAGGAGCTGAGGCTGCACTGTAAGC
AGAAACGCAAGTTCAGGGCGACCACAAGCTCTGACCACAATCTGCCGGTTACGCCGAATCTGCTGAAT
CAGAACTTTACGCCCACAGCACCTAATCAGGTCTGGGTCGCAGATATAACGTATGTTGCCACCCGGGAA
GGATGGCTGTATCTCGMWRRMRWMMMMRMWRWWKMWMMRWRMRMMMWWRKMRRWW
MMRMMMKRRRMRMMMRMMWRMMRMMMRMRYWGMMGGGTAAAGCCCTRTTWMWRGMR
MTMMRRMRMMARMAMMMAMMTRMMRGRMWMMWMMMMMRMWMYRMWMRWRRW
WMMMMRWMMTRMGCMWACGATTACCGGGTCATACAGGAGCAGTCTGGTCTGAAAACATCAAT
GTCGCGTAAAGGTAACTGTTACGACAACGCTCCGATGGAAATCTTCTGGGGAACGCTGAAAAATGAAA
GCCTGAGCCACTATCGTTTTAAGAGCCGGGATATTTCGTCAGCATACGGGAAAACTGATTGAGGTCGG
CCTCAGTTTTGAGGCCTTTAGCCAGTTCAGCCGCAAGGGCTTTAAGCTTCTTTTCGTCCATAATTTACCT
GTCTCCGTTGTTGGAGTAAACATATCAAAAACAGGCAATTACACAATTTAAATTACAGTCTCAAAACAA
ACCCAACGGTCAATATAGTTTACCAGCAAGCTTACTAGCAAAGTGCTAATCATTTGGTATCTTTCGTGCT
ATTCTCCTTTTACGTAAGTACTTAAGGGGAACGTATTTACTACTTTTAAAGAAAGCTCCGTTACTATCTG
AAATGCAATGAGATAATAAACGATTACTCTCGGTTAGTATCTCATGATCATTTACAGACTTTGCATAATC
GCGAATACTATATAACAGAGTTTTAACATTACAGGATGAATAGTCTCTCTTATCAACTCCTAATAAAAGA
AGGATTATGGTCAGTCGTTTTTGCTGTCTTATCATATATTATTTAGTGCAGTTCCTAACAAAAACATAAC
AATTTTCTACAATTGATCCTGTGACTACATAAATATCTTCAACAAAAATATTTATATCAGCAAAAGAGTT
ATTATGAATGATTATAAATCATCAGGTGTTTTTTCAACGACCAACTCCAGAGCACAACGATAAATGTCTT
TTCTAACCAGGTCACATTCAGATGAAATCTTTCTGAGTAAAACATTTATTATATCCTTATTGCTAAGGAA
AGATATTGGGTTCAATACCTCATTAGCAATGGAGTTAATGATGGAAGATTCTATTTCAAATTCAGACTG
ATCAATACCTATTTTATTACAGAAATTAACTCCCATATAACACATCTCCTTTACTTATTAATGAGTGTTTTC
AACCTGCTCAAGCCAAACAATAAGACGTAGGACTTCATCCAAGTGTTCAAAATCAACAAAACTATATTTT
GTATGTGTTTTATATAGTTTTCTAGCTAATTTCACATCACGCACAGTTGGTATACCAACTTCATTTGCATA
TTTTCTGACAGCCAAGGCACACTGGTTAGTTTCAATGAGAGAAATAAAAGGTGCAGGCGCTATTTCTGG
ATTAAAATAAATACCAATTGCAATATGAGTCGGGTTCATTACCACTAATTTTGAATTACGTATATCAGAT
TTAGTCTGCTCTGAAAGAATCTCGATATGCAACTCACGCCTTCTCGACTTTGTCTCAAAGTGTCCCTCTTG
CTCTATATATTCTCTTTTTATCTCCTGTTTATCCATCATCATATCTTTCATGTATAAAATGAACTCAATCAC
AAAGTCAAGAATAATAACAAAAATAGAAAATGCCAAGAAAAATAATATTATATCCTTAAACAGCCCCCC
CCAAATAAGATATAAGCCATCAACACTAGAAAACACCTGAGAAAAAATTATTTTTCGGTCATTAATCCA
AAAGAAATAGGTTGTTAATGCTAGAATAATAAGAAGCAGAATGCTTTTGAAAAATTCTTTTATTGTCTTT
ATACTAAATATTTTTTTTAACCCTTTAACAGGGTTTAATACTGAAAAATCAATCTTGATAGCTTTAGTCGC
AAGAACAAACTTTGTTTGAACCAATGTTGGCAACACAGCCGAAAGAACACAGAAAAAAAGTGGGAAG
CCAATTATCTTAAAAAAGACAATAACCACTGCAAAAAAGTATTTACCCTCATTAATTTCGAAGTCATTAA
TTATTACATATCTGTATAAGAGCATTACATCACTTAAGGAAAAGAATGATATTATAGTAAATGTCCCTAC
CAGAATAATAACAACAGTCGTTAAATCCTTAAATTTAAATGACTGTCCTTTTTTTGCGGCATCCTTTAGTT
TTTTAGGTGTCGGCTTTTCTGTTTTATTTGCCATAATATTTTATCTAACAAATAGATTTGTGAAAAATTTA
TGTTCACCGAGGAAAAATTGAACTTTAGAAAAGTAAATAGTAGAAGAACAGATGAAAATAATAAATAT
TGCAAGTAAACTTTTAATAGTTAATGATACGGAAAAAGCATTCATCTGCGGAGCAAATCTCGATAATAC
ACCAAGTAATACTTCTGATAGTAACAATACTATCATAACAGGACTGGCTAAAATAACAGCCTGACTTGC
CAATAAAGTCAGAAATGTTAAGATATTTGAGACACGGAAAGAACATTGAGAAAATAACGGGCATATAT
TATAAGACAATTGTATGGATTCTAAAATAAAGACCATACCACCACTGTATAGAAATACAACTGCAGAAA
AAAGATTGAAAAATTTTGCCAACTCAGACGTATCAACACCATTGGCAGGATCAATTGAACTACTAAGCG
TTGCCCCACGCTGGTTGTCAATAATGCTACCAACAGCATGAAATATCCAAAAGGGAAGAGACAAGCAA
AAAGAGAGAAGGAGGCCAACAATTATTTCCTTGAACATAAGGAAATAAACATGTTCAAAAACAGAAGA
ACCTATGTCTACCTTACCAGAAGTAATTAATCCTGATGCAACAAGAAAAATCACAGGTATTCTAATAGAT
GGAGAAATTATACCGTTATTTAAAAATGGAAGAAAGAAAAATAATGGAGCCAATCTAAAAAAAACGCC
GTTCAGGAGTATTAAAAACACATGAATACTTTCGAACCAGCTTGAAATGTCCATCAAACCCCACTCTTAA
TTAAAAACATTATTTCATGACAAAAAGACAATAAAACCTCACCATACCATCCAGAAAGAAGTAGCAAAG
ATATTGAGACACCTATAAGCTTTATACCAAAAGGAAGTGTCTGCTCTTGGAGTTGAGTCACTGTCTGTA
ACAAACCAATAGATAATCCAATAACCGTAGCTATACCTACTGGCCATAAGGAAAAGATAAGGATTAAAT
AAAGAGCCTTATTACCCATATAAACTATATCAGACACATTACCTCCATCATCTAAGCAGGAACATTGATA
TATTGTTCAATCAAGGCCTTAGACAGGATACCCCACCCATCAAGCGCAACAAATAATACAAGTTTTATTG
GTACTGATATTGTGATTGGACTCATCATCATCATACCAAGTGCAAGTAAAATGCTTGAAATAACAAGAT
CAACAACAACAAATGGCAAATATAAATAAAAACCTATTTTGAACGCATCTTTGATTTCACTTAGCGCATA
TGCAGGCAAGAGGGAAAACAATGAATAATCATTATTCTCTGCACTCTTTAAATCAGCATTTTCTTCTTCA
GACCTTTGGAAAAAACGTGCTAACTCAAGATCTGTATGCTTTTTCAAGTATTGCTTATATTCCATCAAGC
CAGAATCAGAAAAACGGACAATATCAGATATTGTATCAAATTTTTGAGGGCCATTTAGATAATTTTCAT
AACCAGCTTCAATAATGGGTTTCATCACGAACAAAGCCATAATCAATGCAATACCATTGAGAGTCATAT
TTGATGGAACCTGCTGCAAACCAAGTGCATTTCGTACCATTACAAAAACAATAGAAAACTTTATATAAC
AGGTACCAGCAGCAACTAAAAATGGTAATAGCGTAAAAAAAGAAAGTGTAGCGATGAGGGACATGTC
ACTCAGCATGAGATTACTCCTTTACCATCCAAGAACTAATCTCGATACCATAACCATCCTCAATAGAAAC
AAGTTCCCCATGCCCAACCAAAGAACCATTTACTTTAATATTTACATGTTTAACTATGTCATCGGGAAAC
TTGAATAATTCGTTTTCTACATACATTTTTAGTTCATTGATTGTCATATTTTTTTCTAAAAGAATAAAGTCC
ACTTTTACATTGATATCATCATAATTGAATAATGCCAAAGAAACTTCTGTGTGTTCTGACTCACCATTACT
TTCTGACAGATTAATTTTTGCCTCATTATTATCATTCACAATATAATCCCCAATAATAACTTGATTACACA
ATAATAAATTATAAAGTTTCTGAACTATGATTATATCACCAGTGCGAATTCTTTTTAAAGTAGCCAGCCT
TACATTAGATGAACCTAAGCAAAACTCTAAAAGATGATGCAATAAGTCAAGAGAAACTTGATTACGTAA
TACCGGCATCTTGTCAAAATAAATAATACCTAAAGAAGAGGAAAAACAGCATAAATGACTCCCAGTAGT
TGCATACTGGATTTCATTCGTGATCCGGAAAGTAATCTTATCAGACAGTTCTGGAATCGAAAATACAGA
TGACGACTCTATAAAGACACATTTTGATAAATGCAGTAAAGATTCTTGTGTAGAAAATCCTGAAAATAC
CTCGTATTTTTTTTTCAAAAAAGATAATGTATCAATACGAATTACCCAATCCCCACACACGGAAGTAAGA
GTGACTAAACTTTCACTGCCTGTAAATTTATTTTTGAATGTCTGAATCGAAAAACGCTCGCAGAGTTGCT
TTGCATATAAAATCTGTAGTTTTTCGTTAGCGTCAAAATGTTTAATTCTTAGCATTCTTCTTCACTCTGTTC
ATTATATCCTGTACTATTATTTTTATTCCCCTTATTATGTTGACTGCTATCAAATCTATAATTACCCTCAGC
ATCTTGTTTCAAAATTAAGTCAAGTTTATTTCCTACAAATTGATCTGACGGTTTTAGAACAAAAGAACCA
GACTCCACTGAAATACTAACAGAATGTCCAGCCCCCCACTGTTTAAAGTGGTAAGTCATTTCACTATTTT
TTTGAAAAACATTTTTATCATTTTCAACTATTGATTTTTCTTGCAATGACATGGATGGGGTTATGTCTGCA
GTATTTTTATTCATTACAGGCAGCTCAAATGATTTAGCAACACTTCCATTTAGTAAAGAAATGATGTCAG
CAATATTCTTATCAATAGACAAGCTATGATTTTTTAAACCAATTTGACCATTTCTATTATCAGATTCTAGC
AGATTATCAGGGTTTACAGAAGAAGTATGACTTCTTTCGAAGTTCTTGTCTGCATTTCCACTCTTATTATA
TGAAAAGATCCCCCTCATATCGCCGATATTTATTTTTTCCCTTGCTTTTATATCTTGATCTGGTATCAAAAT
ATTTTCTTTAATGGCCATTGATTCTGGAATATCAGAATATGTCGCTCGTTCGTTTCTAAGTTCGAACGCAT
TTTTAATGCCACTATAAATTTCTCCTTTATCATGCTTATGTTCAAAGTGCTTATCAATAGTAAAATAGTTA
GATGCAATTAAAGAGTACTCAGGAATTTCAACCTTCCTCTTCTTCTTCAAAACGAGACTATCAATATTAT
CTATTGAAGATAGTTTCTCACATTTTTCTATCTGTTTGTCACTACTAAAATTTAGGTTTATATTATCTAATG
CCATACTTCATATCCTCTTGTGATAACTCATCAAGTATCAACTGTTTGGCAAAGTTATTTCTTTTAATCAA
GCCAATCCGTTTACATATTTTACTCTCTTTATGAAGCAGTTTTCTTTTAGAAGCATTTGCCTCATCAATATC
TTTTTGAATTTCTTCAATTCTTCCCTTTAGAAGAATTCGTTCAGACTGATATTGGATAATATGCTTTTTTAC
TATAGCGATCTTACGTCTTGTATTGAAAAGCTGACTAACACTCTGAGCGCCTACAGGTTCTAAGTCATTC
TCCAGTTGCAGGAAGTATATAATCCTTTTATCTAATGCCTCCACATCTCGATACAAAGAGTTTTTTTTTTA
GTGTTAATATGTGTGCTTCAATTAATTTAACAGACCGTTGATATTTATCTTTGATTTTTAAAACCTTATCT
AATTGTTTCACCAATAAGCTCCATTGTCTGCTCATAAGTAAAACCGAGTCGATAATCTTGCTTCAGAAAA
CTCTCAACAACAGATATTTTGTTGTAGATTTTATCCTGAGAGGCATTCTCCCCCGGTTTGTATTCACCAAA
GTCAATAATTGTCCTTAGCTCTTCTATTTCAGAAAGTAGCTCCCGAAATGCAGCGGCCATAATACGATGT
TTTTCATCAACAACCTGTGTAAATACCCTGCTTATACTTTTTAAGGAATCAATTGCAGGAAATTGTCCTTT
TTGGGCTAGATTTCTGCTCAAATATATATGTCCATCTAAAATGGATCTTACCTCTTCAGCTAATGGATCA
GCAAAATCATCATCCTCCAAAAGAACAGTGTAAAATGCAGTAATAGAGCCACCTGCCTTTAACTTTCCT
GGCCTTTCAAGAAGTCTGGGTAAGCTATCAAAAACCGAAACCGGATAGCCTCTTCTGGCAGGTGATTCT
CCAGCGGCTAAGGCCACATCACGTAATGCTCTGGCATACCTTGTTAATGAATCAATAAAAAGCGCTACT
TTATGTCCTTCAGTCCTAAAAAATTCGGCTATAGCAGTGGCTATATATGCAGCATTACAACGATCAACCG
AAGAGTAATCCGAAGTTGCATATACTAAAACACACCTGTTTTTTTTCTCAGAGTTTTTCAAATAATCAAC
CGTTTCTGTAACCTCTCGACCTCGCTCACCAATTAACCCAATAACATATATATCAGCACCACTATGTTCAA
TGAGCATATTCATGAGAAAAGTTTTGCCACAACCAGCTGACGCAAAAATCCCCATTCGCTGTCCTTCACC
ACACGTGAGTAAAGAATCAATAACCTTAATACCTGTTAAAAAAGGCTTCTCAATTGCAGCCCTTTCACTA
TATAGCGGAGGAGCATTATCTACAGGTCGATAAAGAATTTCACTGTTATCCGTAACAGCAAACTTATCA
GTAACCTCCCCTAAAGGATTGACTACTGCTCCCAATAATCCACGGCCGACTTGCGTATGAAGAAACTGG
GCAGTGGGCTTAATCAACGTTTGCCGTGAAAGTCCACGAGAATTTCCAATCAAGCTTAATATTGTTTTTT
CATCATGAAATCCTACAACCTGAGCTCTTGCAACAATTTCATTACTTTCAATTCCAGCCTGAATGTTACAA
ATCTCACCAATCGAAACATCGCTAAGACTTGTTTCCAAGATTGGCCCCGAGATTCTATTAGGAAAAGAT
AATTGAGTGAGCAATTTTGTATAGCTCATTGATTATAAGACCTCATTTAAGATTTCCATCCTCTGATAAA
ACTCATGCAGAATCTCTGCAAAAACAATCCCATCGTGAACGTAGTCATCTTTAATAACAACTCTTAACTG
TAAGTACTCATCAGAACGATGAAGCTCTACCAGCTCATTAATGCTATAACTAAAGTTCATTAACATTAAA
TTTAATATGTTATAAGCAGATGACTGAAGTTTTACATCACTTGGTGCATCAAAATTAGCCCATAACATAA
CCTGCTCATTGACAAGAGCTATATTAATAGCAGGCATACTATCCAAAGAAATGGTAATCGCTGAATGGC
TGTCTAAATCTGTAATAATTGATGGAGGACAACCAATCGTGAAAAGACTATCTCTAACTAATTGAACTA
AATTAATGTTACTCATATTTAAACCTCACTAAATAGTCTTTAATACATTAATGGTATATGCTTCATCAATC
TCAGCATACGATATAACGAGTATACTCTTAAATCTGTTATCTATAAGTCTTTTAACAAACCTACGTATATC
TACTGATACCAAAAGAACGAAATTTTTTTTTGCATTTCTCAATTCTCTCAAAGCATGTGCTAAAGTTTCCA
TTACCTCATCCGAAACCTCTATATCCATATTCAAGAAAGAGCCACCAGAGGTTTGCCTTATCCCTTTTCTT
ATTGCATCCTCAATATATCCGGAAAGCATCACAACTTTAATCTCACCAGAAACAGCTATTTTACTACAAA
TATACCTAGAAAGTGATGCACGGACATGTTCAACTAATGTTATGACATCTTTTTCTCTGGGAGCCCAAA
GCGCCAAAGACTCCATAATAAGTTTTAAATTGCGAACAGAAATATTTTCTCCAAGCAATCTTTGTAATAC
CTCAGAAATTCTCTGTATAGTCACATGTCGGAAGACTTCCTTTAATAGATCAGGATACCGATTTTCAAAC
TGATCTAACATATTTTTTGTTTCTTGTATACCAAAAATCTCATTTATGTTGTTTAATAAAGCTTGTGACAA
CTGGTGATAAAATTCATCTTGAGCACTTTTAATTTTAGCATCAATATTAGTTAGATTTTCTGTATATGAAA
CGTCTACCCAAGAAATAACACGTTCATTATATGAAGTTGAAACAACAGGAATACCTAGAGCATCTATAT
CTCCATTTTCATCTGTAATACACACTTTGTCAAAATATATATTAAAACTATCGGCACGCACCTCATTTATT
AACAAAACAATATCATCGACCTTAAGCTCATTGCTTGTTCTATATAAAATAGTTGGAAGCCTGACACCAT
AATCAATAAAGAACTGACTTCTTATCCTTTCAATAAGACCTTCCATATCATTTGCATTTATCTTATTTTCGG
CAAATAATAAAATTAAAGGAACGGTTTCTGAACTAATAGCATCCAGATTTTCTATCATTGCCAAAGATG
AGTCATGGCTATTATCAATATCAAATGTACCAGTATAGCCACTAGAATCAGACTCAGACAGACTTTTTTC
TTTTTCTACGACCTTTTTATAATAGAATAAAGCCGTCAAAGTAACTGCTATCAGGAAAAAAACAAAAAAC
GGAAAACCTGGCAACATCCCTATCGCCAAGGCAAGAGCTGATGTAACAATGAGAACAAAAGGATTCCC
AAATATCTGGGACATTATGTTACGTCCCATATTATCGCTATCACCATTAACTCTTGTAACGATAAATCCTG
CACTTATAGAAATTAACAGAGCAGGAATCTGAGAGACCAATCCGTCACCAATAGTTAGTATGGTATAAG
TAGATAATGCACCAGATAGGGACATTCCATGTTGACTCATACCAACAGAAATACCTCCTATTAAATTGA
CAAAAATAATAATGATGCCAGCAATTGCATCACCTTTAATGAACTTCATTGCTCCATCAAATGAACCATA
CAATTGACTCTCACGTTCCAGAATACTGCGTCTTTCTTTAGCTCCTGCGGCATCAATAATTCCAGCCTTTA
AATCCGCATCAATGCTCATTTGTTTCCCCGGCATACCGTCAAGAGAAAAGCGAGCAGCTACCTCCGCAA
CCCTTTCAGATCCTTTTGTTATAACAATGAACTGCACAACAGTTACTATTGAAAAAATAACAAAACCTAC
AGCCAGACTATCACCAATGACAAACTGGCCAAATGTAGTAATAATTTTACCCGCATCCGCATCAACTAA
AATTAGTCGACTAGTACTTATTGATAAAGCGAGACGAAATAATGTCGTTATCAACAAAACCGAAGGGA
ATGTAGAAAAACTCAATATTCTTTCAATATAAAATGATCCCATAAAAACGAGAATAGCAAGTACAATAT
TAAGCCCTATTAAAAAATCAACAAGGTATGTAGGTAATGGTATGATCAACATAGCAATGATCATAACCA
TAAGCACTAATATAATTAATTCAGGCTTAGTACTTACTTGCTTAAGAAAAGACTGGATCACTTTCATCTC
CTGTTATCTAGAAAGCTCTTTCTTGTATGCACTTGTGACAAGCTCTCGTAGAATATTGATCGCAATTTCTC
TTTCACTTTCACTTTTAAATATATCATGAGGACTTTCATTAACAGAAGATAGATATGTTTGATACACTACA
GAATCCTCATGACTGGCTCGTGATACTTCTATAACAGAAGTAAGAATTTGCTCACTTTCACTTGGATATT
GAAAAATATATAATAAAGTTAGCAATAGCTGCTCTGCACTTAAACTGCCATCTTTTAGCAAGCTTGAGG
ATTCCAGTTTCTTCATAAAAATTACGTCTGAAGTTCTAATCATCTTAATTGCAATTAATTTAGATAACACA
AAACCAAACTCATAAGCATTACAGCTCGGTTTCTCAGAATCCATGTCTACAATCAGTGACTGCTCTACAA
AATTCACAATTGTATGTCTGTGATTAAAACCAAAATTACCAAGCCATTCTATATACTGATCTGTTGTTGAT
ATGTTCCCCATGATAAAACCACGATAACATGCACGCAAAAGCTGTGGTTCAAGTTTCATTTTGTTGCCAA
ATAATTTAGCCTGTATAGCTGAATTTATTCCAGCATTAACACTTTTTTCACTACCTGATATTATCTCATTAA
TAATTTTAGTTAATGATTCGACTTGTTCAGCAGTAAGATCTTTTTCATTCAAAAGTTCCCTTAATGCTAAG
ACTTGATCACTTGGATCTTTAAAATATCTCTTTAGTCTGTCTATAAAATCCCGATCTAGAGGAAGGTTAT
CTTTTAACGTTCTCTTTAAATCAAAAATCTTGTGATTAATTTCATCTTCTTCCCCATCTAAAATACGTTCCT
GACTATCACTATTTGTTCCTTTCAGTTTCTCAAGGTCTCTTCTGTTTATAAATGACGAAAGAGCGGAAGC
CATCTCATCAGATGAGTCAATATACTTGCTATTAGCCAAGCCAGAATCAAGCTCTGCATCAGCAGTCTCA
TCTCCATCATCTGAATTTGTCATTGCATTCAACTTATCAATGAATGCAGAGCTAAAAACTCCTGTATTTTT
AACATCAAGCATACATCACCTACTTTATAGTTAGTAATTTAAGTATGAAACCAATGATTTTTCGTCTTCCA
GCAAAGTAGTTTCAGATGGGATTATTTGTGTAGTCTTTTGTATTTCTCTTTCAGATATGAGGCTTTTATAT
TCTGCAGTATTATAATAACTTGACTCCTTTATCTCACGGGGTTGAATCAGAAAAACCCTTACAATATTAC
TGATATTACTTGTTTTATACTTGAACACATTACCGATTACAGGAATAGAGGAGAGAAAAGGAATAGATA
CTATTTCGTTAGAGTTCGTTTCATGCGTATAACCACCTATCAACAAACTTTTGCCTTGTGGAACTCTCGCT
ATTGTACTGATTTTGGTCCTTCCAACTTCTGGAAGGACAGATGTATTTTCATTATTATAATTGTAATTTGA
CTGACTATTTCCTGTTCCATCCTCTATCGTTAAAGACATCTCAATTTGTCCCCGAGATGAAAAGCGAGGT
ATAACATTTATCAATGTTCCATACGTAACATGCTCCAACGAAGAATTGCGCTCACCAACTAATGACACAT
AAAAAGTCCTGTTATTATCAAATATAGCCGGAATATTCTCTTGTGTCAAAATCACCGGCCTTGAAACTAC
ATTTGCTTTCTTTTTCTGATTTAATGCCATAACTGAAGCAATAAACTTATTGCCGTCCAAAGTGCTTATCG
ATGCTGATGAGCTCATATTAAATGACGCACCAAACGAATCACCAAATGAAGCCGTTCCTTGCCAATTAA
CACCAAGGTTATTTAATTCAGATTTATCAATATCGATTATCCATAATGATAATTCTATATGTCGTTTGGCT
ACATCTAACTGAGTGATAATATCGCGAATAATCTGTATTTGCTGATCATTACCTTTGACTAATATAGAAT
TGGTTTCTGGATATGCAATAAGTGATACATCCTCTAATATAGAACTATTGGTAACTGAACTAAATGAAA
AGTCATTACTATCCTCAGATACTTTCTGTGTAATATTAAAGGGTGGCATCGGATCATTTTGGGCTTGTCT
GTTACTCAGAGCCTTACCATTATTTAATAAACGTTCTACAACAGTAGCCACCCCTGGGATGACAATATCC
TCCCCTCTCATATTATAAGTTCTGTCACTAACAAATGTATTTTTTAATTTTATAACCCCAAAATTAACTTTA
TCTGTCCCAATAGAAGAAACCTGCTTATCAAGCAAAGTTGCTGTATTAGCTACCAGTTCAACCAATGCC
GGAGGACCAGATATATAGAATGTTTTATCACTAATATTTCCTCTTATAGGATATCTGTGATCATAAAGGT
TAGCATCCTTCAGATACTGAATAAGATAATTAAGTGATATATTCTCCAGCAAAATAACTTTGCTTATTAA
TTCTCCAGAGTCATATATGTATAATGCATTTCCATCTTTATACCATATGAGTCCCACAAGTAATGTCAGTT
TTTCAAGCATTTCTTCTGGATTAGATAAATCAAACTCCCCAGATATTCTCTTTTTTGCAGCTTGTTTGCTA
ACAACAATAGGATAATTTAATAACGCAGAAAAACGGTCAAAAAAAGATCCTACCGTATCACTTTGAGCA
ACATATTTCGCTATGTCATTTTGTTCTAATAAATGAGAGTCAATATTATTAGCATTAACTATCAATGGTAA
CAATACAATCAAGAGAGTCAAAGATTTAATATTAAATTTTTTCATTTATTTTTTTCACTAAAAAAGTAATA
TTCGATAATTCTCTTGCAGAAAAGCCTAGTCGTGTTTTTATCTCATTTGAAAAATGGGATGTTGATGCAT
ACCCATTATTCATGGCAGCAGAAGTGATTGTTTGATTGTGCAAAAATACATCTAATAAACCATTTACTAA
CCGCCAAGTGTTCAGTTGTTCTTTGACTTTTGCCCCTAAAGCTTTTCTACATAAAGATCTAAAATAAGCCT
CCGACACACCATAATGCTCTGTCAGACTCTTTATTCTTACCTCCTTTTCTTTCTCAATAGTTCTCAGAAGAT
AAAGAACCAAATGATATGACTCAGTCATTCGTATCATAGCCAGTGCATTACTTACATTCTGGTCAGATG
AAAAGATGAATCTTAACATTAACTTATCAAGCATCGATTCGGTATTACAACTATCCCGAAGATCATTCTC
ATGATAGAAATGAATACATTTTTCAGCATGGCTATGCTCTACTAGAGCACCACTGAAATAGTCCCAAAA
AGCAAGAAATTTTGCACAACTATTAATGTTAAAGCGATAATGATAGTTCACATTATCACTGAATTTTATA
TTTTGATTTCGGACAAAAAAAAACCCTTCCATGCTGGAGCCAGCTCGATTATGTTACGTTCTTTATCTTTT
ACAAGCTCAACGTTAACTGGTTCATGTCCACTAGAGTAAATGTAAAACATATTACTTGTATTTAGACCTT
TATATTTACTCACAATACAATTATATCCCTCGAATTAATTTTAATTTAACTTCCCCATCCTTCTCTATAAGG
ATGGTATCATTTATCTTATTACCTTTTAAAACAACCCCGTCTTTCAGCATAGAAAATAGTTTATCTATAGC
TATCTTTTGTGCATCCATGAAGCACCCATCTACTTCCTTAACATTAAAGTTGCCCATGAATTTACCTGCAT
TAAAACTATAAACCGCCTTACCACTAATACATTTAGAGTAAACATTTATACTTTTATTGGTTGTATTAAAC
GACCATAATAAATCATTTGGAATAGAAAAATAATCCTTACTTACAGGAACAATATGCCATTCTTTTTCAG
AATTAGATGATGACTTTAAAGCACACCCACTCAGTGTTAATAGCAATATACTTAAAATAAATATTTTCAA
CTTATTACTACCATGTCGAATCATCTGCCTCTCTCCCTGCTTCAAAGACACTAATTAATGAATCGAAGGTT
GTAGTCTTAATATAGAAATCAATATTGTTATATATGGACAATGATGCATAAAATCCCTCATGCAAATCGA
CATATTGATTTGCATCTATGAGTTTATTAAGAAGTGAACTTTTTTGTGCAGCTGTAAGATAAGGAAAAC
GTGAATTGAATAATTGTTCAATCTTATTGTGCAGTTCATCATTATCTATGGAATTTAATTCACTCTGTGAG
AAATATTGCATTATTTCATAGATTATATCCTCATCAAATGATTGCGAAGATGATAATCTCTCATTTAAATT
AACAAATTCTTCACTATTTAATCTTTGCTGCAGAGCATTGCTTGCATTTATTTGATTAATCATTAACAGGA
TTCTCTTTTAACAAGTCTTCTGCAATTTGTCTCAGTTTCTTATGTGCTAACTCTTTGAAATATTTTTTATCAT
TTTTTGTTAATTTTTCCGCAAGTTCTATTTTAACACCTGATATTACATCCTGAGGACTAAACTCCACTACTT
GATTACCACTACAGAATATATAATTATTCCCTTCAGAATATTTTAAAATAATCTTGACATCTGAGCGATAT
TTATGGAGATCAAGTTCTAACTTTTTCCTCAGTGCTAAATTACACTTGGGCAAAACCACTTTTAATTCTGT
TACTGTATTACGCAACGAACTAAGCCACTGCTCCAATAAAGTGCTAATGATCCTCTCATCATGTAAATTT
TCTGAAAGCAAACTAGTTATATAGTCCTCAATATTTCTTCTGTTTTCATTTTGCTTTAACTTCCATTCACAA
ATATAGTCAATAATATGCTCAAAAGCAGTCTGTATACCAATTTGATAACCATCACATACAGCATCTATAC
GCAGTGATTCAGCTTTTTTCGTAGCATCCTTTATAACACAAATTGCTTTCTTCTTACTCTCAGTGTCTCTAT
CTTTTATGGTTTTACATAATTCCTTTTCAATTCTCTTTATAACAACACCATCATAGGCATGATGTCTGGAT
ACCGGTAAAGTGCCTTTTTGCATATTGCATACCTTCATTTAATCGGGACAAATTTCTTTCTACAGGCGTAT
AATTTCCCTCCAAAGCAATTGGATTAAATAATAAGTTTATTCTTTCATATATACCAACAGATATATTTTCC
TTCCACGCAATAAGATGCCAAAAACCACTTCGAATAGCGGCCTCCCCTTGCTTACTGATTTTGCCCCCAT
AATAACTAATAACCATCTCACTATGATTAATTAACCGAGACTCTTGACTACCGATAAAATAAGCAACAGC
TGGAAGTAAATTCCAGTTGGCAACAAACATAGCGGTTACCCCGCTTAAAGGTTCAATATCGCAATTTAG
ATCATATTTATTAATTATGAGATTATTAATCTCACTTCTGATTACGCCATTTTCTAACAGTTCAGAAGGCA
AATTCAACCGATTTCTATTTATATAGAACGCTGGATCAAAAATTATTGAAAGATATTTTTTATAAATTCCA
TCCATTCTTATCATATTTTGTTTCTCTTGAACCACCCTGTTTTGAATGCCCATACCAAAAGAATGACAACT
AGTACAGCCATCCCGAGAAATAAATATATTACTTCATTTGTTAAAAATTCCGATTTAATTTCCTTAACAG
GTTGTACATTTGTATAAACATATTCTTCTTTCGGAGTTAATATGACAGATATATTTTCATACTTAACATCA
GAAAAGGTGTTTTTCAAAAATCGCTTAATATTACTAACTAATAGTTCAGACTCTTTCGGTGAGTCATATA
TAGCGATTACTGATATATGCATCGGTTTTGAAGATATATTTTTTTCTTCAAGATCATAGCTAACATGTATT
TTTGCCGAAATAACACCACCAATAGAAACTAAAGACTGTTCCAGCCGTTGCTCAATAGCACTATATAAA
CGGGCCTTTTCAGCTCTTGGAGAAGACACTAATGAATCTGTAGGAAACATTTGTGAGATATCAACTCTC
TCCGGATTTGGCAAATCGTACATGCGCATCAAATCAACTGCCGATGCAAATGTCCCCTTTTCGACTTGTA
CCGAGATCCCCTGTTTACCTCCATCAACTTTTCTAGCAGTAATATTATGGCGTTCTAGCACAGATATTATT
TCATTTGCCTGTCTTTGAGATAAATTAGAAATTAACTCTTCACGTTGCTCACATCCAATCAACATCAGCAA
CAAGAATAAAATAAAACCTTTATACCTAATCATATAAATTAAGACTTTAATAAAGTTTCAACAGCCGAAA
CTGTTTTTCTTGCTAACGTGCCAGCTAATGATACTCCTATTGAATAATTTGAGAGCGTCGTCTGTAACTT
GGCCAAAGATTCTGGGTTTAATGATTCTGGATTGCTTACCATCTCCATTATTTGTGATACTTGAATATCT
GTATCCATCTTAATATCACTATATTTAGCCGACACGACATCTTCCAGACTTGATATCTCTTGAGATTGAAA
ATCACTGGCTTTGATAATATCAACCTGATTGACTGGATAAATGTAATTCATGAATGCTCCCTATTATCTG
AAGTTTTGAATAATTGCAGCATCAACATCCTTAATCACTTTCACTGTATTGGATTGCGCGTTCCTATATAA
TGTATATTCAGATAATTTACTTTGGTATTCAGCCAGCAACTGTGGATTCGAAGGATTTTTAGCTAATTTA
TCTAGTGCCAATGTTAGTTCACCTTGTAATGTTTGAGTTCCATCATCAAAAGTTTCAGATAATGAACTCA
GTGTCCAATCATCATTCGGTACTGTAACACTCATTTTATCCTCACTTATTITTATCTAAAAAAAACCAGTG
TTTATCATTCAACATTACATAACTGTCTTTGCTGTTAAGATATGATTTACCTTTAAAATCATCATCTATCAA
AAGTACAGAAAACTCAATATACTGATCTCCATATATATTTTTATGTTCAGAAATCAGTCTATTAATGAGC
TCAAGCTTTTCATCTGTCAGTTCTTCTCTGACAGAATATGTAACTTTATTATTTTCACAAATCTCTTTATAC
TGGACATTTACCTTTGTGAAAATACCTTTAGCATCCCCCCTTGCATTATGATCGCTAAGATAGTTGAATTT
AATATTTTTGATATACGGAAATTCAACAATCAGAGCATTTTTCAATTTATCAAGTTCGGTATCTTTTGAAC
TATTTCTTTCTTTACTTAAAAACACCAGTAATTCCGCTTTATCACTCACTAAATTCAACACATACAAATTG
ATAAATGGGAATCGTTGATTGATATATTGTTGAATTCTATTTATTTCTTTGTTTGACACCAGAATATATCT
GCATTTGGTAAATCCGGTTTTATTTAAAGCTACAGATGCCCATACCGCATCATTTTGTGTTTCGGCAAGA
ATATAATTGCACTGTCCAGTGTTGACTATTCCATATCTCTTATCATCTATAATTTCAGCAATTTCTTTTACT
TCATCTTTCTTTAAAGAGAGGGAAAAAATTATAATTAACACAACAATAACAGCAAAGAAAAAAAACACC
GAGTGGTTCTTATACATTCCGTTTAGTATATGGCCTCTCGATTTATCCTCCCTCATATTTTTAAGGTGAAA
AGAAATCCCGTCATATTCAAATGAATTAAATTGTGAATTGATATCAATTTGCTCACCTTTTTCATCACTAA
ATTGACAGATATTATCTTTGCTTATGGTCAAATAAATTATACCATCCGATTTACTATCTGTTACAGGAATA
ATATTTTCTGGGAAATTGTCGTTTCCAAACTCAAGTGTTTCTATCGTCCGTCCGAGAACAATTAAGTTATT
CCCATAATATAACGGAAACTCATCCCCGACACCGTTTGTGAGTTTCACTAATAACCTGAATGGTGCAAG
ATTTGAGTTCTTTGCCTCAGACATAAATCCTCTTATATCCTTCGATTATTCTGCTTGCTCTCTGCTGCTATC
CGCAAATAGCGTCTATAGATATCCTCTGCGTACTTTAATCTTTCTTTTTTCTTTTTCGGAGACGTTCCTGC
ATTATAGGCTCCAACAGCCTCCCACCCTCTTCCATACATCATCATAAACTCATTTAATAACTTTGCTGCAT
AATGTACAGAAAGACATGGATGACTAATTAGCATTTCCTCAGAATATCCCATTTCTCTAAGTCTTTTAGA
ATGAAAGTCATTTATTTGCATTATACCATAATCTTTACTTCCATTGTTATTAACATTGACTGCAAATTTATT
AAATCCGGATTCTTTTTCCGCAATCGCTTTTAACAAACTTGATGGAATATTGTACCTTTCACCAGCCTTAT
CCCAACAATCAGCTCTCCCTAAATATGGGATAAAACACAGCAAAATAAATACGAAACGGGACATTAATA
CCCCTTCATTCTTCGCGCAAATTCATCCTTCAGCCAACGCACACGCGAAATATAATATTCTGTATTAACAT
ATACATCCTCGAATTCATTTATCCCGGTAAGATCTAATCGCGCAATTAGATTTCCGCCTTCATCATCAGTA
GCTAAGCATATCTTTTCTGAGTAATTTAGGCTCAACGCATATATAAGATCATTTATATTTTCAGGCAATG
CACAAAAAGGGCATAATAAATTTATTGAATCTCCTTCTTTTTCAATATATATTAGCACATCATCATCAAGC
ATGATTGCTTGATCATCGATATCAATTAAAGGGATACCCAAGGCTCGGCAAATAACATCTGCTAAATCT
TCCATATATTCCTCTTATACAAATGACGAATACCCTTTCACCATATTCCATATTTTTGAGTCCCCTATTCTT
TCAGAATAAGATAGCTCTAACGAAGATAGCGGCAATTTTTTCATGACTTTATTTCCGGGCACACCAGTAT
TCATCTCTTGGATTTCCATATTACCACTATTCATTAGAATAGTAGAAAATAATCTTTTCTCTTCTGAGGAT
AATTTACTATTTAATTGAGAAAACTGACCTGTTTCATGTTTTCTTATTATTTCTCTTTTAATTTCAGCATCC
TGCATACCTGTTCTGTCCTTCCCACTCTTACAATTCCAGCAAGGTACAGCACCTATAGTATAAGCCAATA
GAGTCATTCTTTGTGACAATTTATACGGCTCTCCATTATCATTTTTTTGTAACTTCTTGTTTATAATCTCTT
TTATCTGGTTAGCCAGATATATCACATCATTCTTACATGGTGGATTTTTTTCTATTGCTTCTGCAGCCCAA
CCTCCAATCACACCATTTTTGAGGAAATTATCCCCCAACAAAGAACATATTGATTCATCATTAAGCTTGT
CAACATTCCTCCAGCCTAAGCCCATTTTTAGCGCTAATTCATTTACACCAAAGTTAAATGTCACCACTTTT
AGATTAACGCTTACTTCTTTCAGAAGACCATCACTATTCCGAATTAATAATTTCGTTGGCTCCCCCCTTTT
AGAGTTTAACCCTTTTAATGCGTTCACCTGATCTTTTAACATACTCTCCTCTCCCCCGGTTAAACTGGTCG
GCGTCAGAAGAGAAGTCGAAACAATCTTTAAATCTACTGTTTTACCAGACAAAGCCTGTGATAATAACT
CTGGTCTGCTATATAATGCTGCGCTTACTAACTCCTCAGCTTTATTACGAGCAGCAACAGCTCTTTCAGA
TGAGTTTTTTTTCAATCCATAGGCTGATATAACTCCATGTCTGATACCCGAAAAAATTTCCTTCCCCTCGT
CATCCACCACTTTTGAAAGCCACATATTCGCGATATGATCACTCTCTCTCGTAGAAGCACAACAAATTCC
TTTTCCATTATACTCTTTGACAAATATATTTTTATTCCCTATCTTCATATGAGAGGCAGGTGTTAATTGAA
AACCATAATTTTTCCCATGATGACTAATATTCTTGTTGACCGGTCCCCAATTCTTATTATTTAAATAATCA
GTAAATATTTGTTTTATTTTCGTTTGCAATTCCTTTTTATTTTTAATGCCTAGCTGTGCTCTTATCTCTTCAC
TTATTACTTTATTAAGCTCATTAATATGTCTTTTATTAACTTCACCAAGTTTATAGGTATCTAATCGTTCCA
TCATGGCTTTAATTTTTGACGTATCACCACCTGCCAACTTTATTGCCTCGTAAAAAACTTCAGCTCTGTCA
CGCGCAAACAAAAGGCTTTTTCCTGTTAAGCTTGTTTGGTTCTGTAAATATAATCGATTAAGCGCTTCGA
TCCCCTTATTTCTTTCTCCATTTTTAACTCTTTGATATGAAATCACGCTTACACCTTTGTGCTTTCCGGTTTC
TTCTGCGCCTTTATAGGAATCTCCGCTTTGAAATGAAACCTGATGCAATCCCAAATTAGTTATGTGCATT
TAATTATCCTCAGTCATAAATGGGGCATTGATTGCAGTTTTGTACCTCGTITTACAACAAAAAAGATACT
TATTCAACAGGGTTCTCTCTTTTTCTATAACACTCCGAGCCTGATATATCAGACACTTTAACTTGTGGGAT
TTCATGATGAAACGAGCACTACATATGCTCACGAGGTACATATTATGTACAACAAAAGAAAATTTGATT
ATGTTAACAGGCTCAAGTTTCTAAATTTATTAATTGAATATATTATATATCAAATACATTTTACATCCCCA
CAATCACCACGTAATGGAGAGTTAATAAAGTATGATCCTCAAAATTAGCAATTTCATTGACGCAAGCAA
TACAAAAGGGCCTATACGCGTTGAAGATACAGAGCATGGACCTATATTGGTAGCACAGAAATTCAACC
TAAAGGATCTCTTTTTCAGAACATTAAGCACTATTAATGCAAAGATAAATTCACAGATATTAAATGAACA
ACTAAAAAATTATCGACTAGCGAATCAAAAATCCTTATTGCTTTTCCTCAAGACATTAGCTAGCGAAAAA
TCTGCAGAAAGTGCGTTTGCCGCCTATGAAGCAGTCAAAAATTCAATTCAACATTCTTTTACAGGTAAA
GACATCAAATTAATGTTGAATACCGCAGAGCGTTTTCATGGCATCGGTACAGCCAAAAATCTTGAAAGA
CATTTAGTTTTTCGTTGCTGGGGAAATAGAGGCATAACCCATTTAGGGCATACCAGTATCAGTATAAAA
AACAATTTACTACAGGAACCAACTCATACCTATCTCAGCTGGTATCCCGGCGGGAATGTTACAAAAGAT
ACAGAAATAAACTACCTTTTTGAAAAACGCTCAGGTTACAGTGTCGATACCTATAAACAAGACAAACTA
AATATGATTTCAGACCAAACAGCCGAAAGACTTGATGCAGGCCAAGAGGTTAGAAACTTATTAAATTCA
AAACAAGATCAAAATAACAACAAAAAAATATTTTTTCCCAGAGCTAATCAAAAAAAAGACCCCTATGGT
TATTGGGGGGTCAGTGCTGATAAAGTATACATTCCATTATCAGGTGATAATAAAACAAAGGATGGTAA
AATCAGCTATAACCTTTTTGGCCTTGATGAAACAAACATGTCAAAATTTATATGCCAAAAGAAAGCAGA
TGCTTTCAGACAATTGGCAAATTATAAATTAATCAGTAAATCTGAAAACTGTGCTGGTATGGCACTTAAT
GTTCTTAAAGCAGGAAATTCTGAAATATACTTTCCACTCCCTGACGTTAAATTGGTTGCTACACCTAACA
ATGTATACGCGTATGCAAATAAAGTTAGGCAACGCATTGAAAGTCTCAATCAGTCTTATAACGAGATAA
TGAAGTATATTGAATCTGATTTTGATCTTTCCAGATTGACTCAATTACGACGTAGCTATCTTAAAAGTTTC
AATAAAATTAACCTTATCCACACCCCAAAAACATTCAAGCCTTTATCAATTTCACTTTACAAACATCCTAC
TGAAAATGTATCTTCAGAAGACTTTGATGCGGTCATCAACGCTTGCCACTCATACTTAGTAAAATCAGCA
CCCAGCAATATGTCTCGTGTATTAAACGAATTAAAAACCGGAGCAACAGATAAAAAAGAAGAAATTAT
CGAAAAGTCAATAAAAACTATTGATTATTACAATTCTCTTAAATCGCCAGATCTTGGAACCAAACTTTAC
ATTCACGACTTATTACAGGTCAATAAGCTTTTATTGAATAACTCTCATTCTAATATATAGAAGGCCATAG
AAATGTGTCGCAAACTATATGATAAACTTTATGAAATAACAGGTGCAAAGCTTGATTTTAACGATAAAA
ATCAAGCCTTTATATTGCTCGAAGAACAAATACCTGTTTGTATAACAGATAATGATGAATACATCTTTTT
GACCGGACTTCTTAATGAACATGAACTTITTACAGAAAATATTATTAATCCAGAACACATACTCATCCTT
AACTACTCTTTGAGTAGAGATTATGGTTCTTCTATCTGTCTTCTCCCTGATACTCATCAATGTGTTTTGAC
CAAGAAACACTACAAAAAATATCTTTCACCTGATGAACTCATTGAATCTTTGTATGAATTTTTATTTTGTA
TTAAACTAACCATTGCAAACATCACTTCAGAAGTGAACTAACATATAGGGGGTATCATGCAAATTCTAA
ACAAAATACTTCCACAGGTTGAATTCGCGATTCCTCGCCCATCATTTAATTCGTTAAGTTACAATAAGCT
TGTAAAGAAAATATTATCTGTTTTTAACTTAAAACAACGCTTTCCTCAGAAGAATTTTGGATGCCCGGTG
AATATAAATAAAATCAGGGACAACGTCATAGACAAAATAAAAGATAGTAATTCGGGGAATCAACTATT
TTGTTGGATGAGCCAAGAGCGAACCTCTTATGTCTCTTCAATGATAAACAGAAGTATAGATGAAATGGC
CATACACAATGGAGTTGTTTTGACCTCAGATAACAAAAAAAACATATTTGCAGCTATTGAAAAAAAATT
CCCGGATATAAAACTTGATGAAAAATCAGCACAAACATCTATCAGTCATACAGCACTAAACGAGATTGC
CTCATCTGGCCTCAGAGCAAAGATTTTAAAACGCTATAGCAGTAACATGGATTTGTTTAACACACAAAT
GAAGGATCTTACAAATCTAGTATCCAGTTCCGTCTACGATAAAATATTTAATGAATCAACAAAAGTTTTA
CAAATAGAAATAAGCGCAGAGGTTCTAAAGGCCGTATACCGTCAAAGCAATACAAATTAAATCTGTTTA
TATTAAATCTTATACTTATTAAAGTAAAGCAGACAAAAAGGAGACCTTATGTCTTTAAATATCACTGAAA
ATGAAAGCATCTCTACTGCAGTAATTGATGCAATTAACTCTGGCGCTACACTGAAAGATATTAATGCAA
TTCCTGATGATATGATGGATGACATTTATTCATATGCTTATGACTTTTACAACAAAGGAAGAATAGAGG
AAGCTGAAGTTTTCTTCAGATTTTTATGTATATACGACTTTTACAATGTAGACTACATTATGGGACTCGC
AGCTATTTATCAGATAAAAGAACAGTTCCAACAAGCAGCAGACCTTTATGCTGTCGCTTTTGCATTAGG
AAAAAATGACTATACACCGGTATTCCATACTGGACAATGCCAGCTTCGGTTGAAAGCCCCCTTGAAAGC
CAAAGAGTGCTTCGAACTCGTAATTCAACACAGCAATGATGAAAAATTAAAAATAAAAGCACAATCATA
CTTGGACGCAATTCAGGATATCAAGGAGTAATTATTATGCATAATGTAAGTACCACAACCACTGGTTTG
TCTCTTGCCAAAATATTGGCTTCCACTGAGCTTGGAGACAATACTATCCAGGCTGCAAATGATGCAGCT
AACAAATTATTTTCTCTTACAATTGCTGATCTTACTGCTAACAAAAATATTAATACAACTAATTCACACTC
AACTTCAAATATATTAATCCCTGAACTTAAAGCACCAAAGTCATTAAATGCAAGTTCCCAACTAACGCTT
TTAATTGGAAACCTTATTCAAATACTCGGTGAAAAATCTTTAACTGCATTAACAAATAAAATTACTGCTT
GGAAGTCTCAGCAACAGGCAAGACAGCAAAAAAACCTAGAATTCTCCGATAAAATTAATACTCTTCTAT
CTGAAACTGAAGGACTAACCAGAGACTATGAAAAACAAATTAATAAACTAAAAAACGCAGATTCTAAA
ATAAAAGACCTAGAAAATAAAATTAACCAAATTCAAACAAGATTATCCGAACTCGACCCAGATTCACCA
GAAAAGAAAAAATTAAGCCGGGAAGAAATACAACTCACTATCAAAAAAGACGCAGCAGTTAAAGACA
GGACATTGATTGAGCAGAAAACCCTGTCAATTCATAGCAAACTTACAGATAAATCAATGCAACTCGAAA
AAGAAATAGACTCTTTTTCTGCATTTTCAAACACAGCATCTGCTGAACAGCTATCAACCCAGCAGAAATC
ATTAACCGGACTTGCCAGTGTTACTCAATTGATGGCAACCTTTATTCAACTAGTTGGAAAAAATAATGA
AGAATCTTTAAAAAATGATCTGGCTCTATTCCAGTCTCTCCAAGAATCAAGAAAAACTGAAATGGAGAG
AAAATCTGATGAGTATGCTGCTGAAGTACGTAAAGCAGAAGAACTCAACAGAGTAATGGGTTGTGTTG
GGAAAATACTTGGGGCACTTTTAACTATCGTTAGTGTTGTTGCAGCAGCTTTTTCTGGAGGAGCCTCTCT
AGCACTGGCAGCTGTTGGTTTAGCTCTTATGGTTACGGATGCTATAGTACAAGCAGCGACCGGCAATTC
CTTCATGGAACAAGCCCTGAATCCGATCATGAAAGCAGTCATTGAACCCTTAATCAAACTCCTTTCAGAT
GCATTTACAAAAATGCTCGAAGGCTTGGGCGTCGACTCGAAAAAAGCCAAAATGATTGGCTCTATTCTG
GGGGCAATCGCAGGCGCTCTTGTCCTGGTTGCAGCAGTAGTTCTCGTAGCCACTGTTGGTAAACAGGC
AGCAGCAAAACTTGCAGAGAATATTGGCAAAATAATAGGTAAAACCCTCACAGACCTTATACCAAAGTT
TCTCAAGAATTTTTCTTCTCAACTGGACGATTTAATCACTAATGCTGTTGCCAGATTAAATAAATTTCTTG
GTGCAGCGGGTGATGAAGTAATATCCAAACAAATTATTTCCACCCATTTAAACCAAGCAGTTTTATTAG
GAGAAAGTGTTAACTCTGCCACACAAGCGGGAGGAAGTGTCGCTTCTGCTGTTTTCCAGAACAGCGCG
TCTACAAATCTAGCAGACCTGACATTATCGAAATATCAAGTTGAACAACTGTCAAAATATATCAGTGAA
GCAATAGAAAAATTCGGCCAATTGCAGGAAGTAATTGCAGATCTATTAGCCTCAATGTCCAACTCTCAG
GCTAATAGAACTGATGTTGCAAAAGCAATTTTGCAACAAACTACTGCTTGATACAAATAAGGAGAATGT
TATGGAAATTCAAAACACAAAATCAACCCAGATTTTATATACAGATATATCCACAAAACAAACTCAAAG
TTCTTCCGAAACACAAAAATCACAAAATTATCAGCAGATTGCAGCGCATATTCCACTTAATGTCGGTAAA
AATCCCGTATTAACAACCACATTAAATGATGATCAACTTTTAAAGTTATCAGAGCAGGTTCAGCATGATT
CAGAAATCATTGCTCGCCTTACTGACAAAAAGATGAAAGATCTTTCAGAGATGAGTCACACCCTTACTC
CAGAGAACACTCTGGATATTTCCAGTCTTTCTTCTAATGCTGTTTCTTTAATTATTAGTGTAGCCGTTCTA
CTTTCTGCTCTCCGCACTGCAGAAACTAAATTGGGCTCTCAATTGTCATTGATTGCGTTCGATGCTACAA
AATCAGCTGCAGAGAACATTGTTCGGCAAGGCCTGGCAGCCCTATCATCAAGCATTACTGGAGCAGTC
ACACAAGTAGGTATAACGGGTATCGGTGCCAAAAAAACGCATTCAGGGATTAGCGACCAAAAAGGAG
CCTTAAGAAAGAACCTTGCCACTGCTCAATCTCTTGAAAAAGAGCTTGCAGGTTCTAAATTAGGGTTAA
ATAAACAAATAGATACAAATATCACCTCACCACAAACTAACTCTAGCACAAAATTTTTAGGTAAAAATAA
ACTGGCGCCAGATAATATATCCCTGTCAACTGAACATAAAACTTCTCTTAGTTCTCCCGATATTTCTTTGC
AGGATAAAATTGACACCCAGAGAAGAACTTACGAGCTCAATACCCTTTCTGCGCAGCAAAAACAAAAT
ATTGGCCGTGCAACAATGGACACATCAGCCGTTGCTGGTAATATATCCACATCAGGAGGGCGTTATGC
ATCTGCTCTTGAAGAAGAAGAACAACTAATCAGTCAGGCCAGCAGTAAACAAGCAGAGGAAGCATCCC
AAGTATCTAAAGAAGCATCCCAAGCGACAAATCAATTAATACAAAAATTATTGAATATAATTGACAGCA
TCACCCAATCAAGGAATTCGACAGCCAGTCAGATTGCTGGTAACATTCGAGCTTAATATATCCAAGAGC
CATAATAATATATGGCTCTTCCTGTAAGGAAATAACCATGAATATAACAACTCTGACTAATAGTATTTCC
ACCTCATCATTCAGTCCAAACAATACCAACGGTTCATCAACCGAAACAGTTAATTCTGATATAAAAACAA
CGACCAGTTCTCATCCTGTAAGTTCCCTTACTATGCTCAACGACACCCTTCATAATATCAGAACAACAAA
TCAGGCATTAAAGAAAGAGCTTTCACAAAAAACGTTGACTAAAACATCGCTAGAAGAAATAGCATTAC
ATTCATCTCAGATTAGCATGGATGTAAATAAATCCGCTCAACTATTGGATATTCTTTCCAGGAACGAATA
TCCAATTAATAAAGACGCAAGAGAATTATTACATTCAGCCCCGAAAGAAGCCGAGCTTGATGGAGATC
AAATGATATCTCATAGAGAACTGTGGGCTAAAATTGCAAACTCCATCAATGATATTAATGAACAGTATC
TGAAAGTATATGAACATGCCGTTAGTTCATATACTCAAATGTATCAAGATTTTAGCGCTGTTCTTTCCAG
TCTTGCCGGCTGGATCTCTCCCGGAGGTAACGACGGAAACTCCGTGAAATTACAAGTCAACTCGCTTAA
AAAAGCATTGGAAGAACTCAAGAAAAAATATGAAGATAAACCGCTATATCCAGCAACTAATACTGTTA
GTCAGAAAGAAGCAGATAAATGGCTTACAGAATTAGGTGGAACAATCGGCAAGGTATCTAAAAAAAA
CGGGGGATATGTTGTCAATATAAACATGACCCCAATAGACAATATGTTAAAAAGCTTAAATAATCTAGG
TGGAAATGGCGAGGTTGTGCTAGATAATGCAAAATATCAGGCATGGAATGCCGGATTCTCTGCCGAAG
ATGAAACAATGAAAAATAATCTTCAAACTTTAGTTCAAAAATACAGTAATGCCAATAGTATTTTTGATAA
TTTAGTAAAGGTTTTGAGTAGTACAATAAGCTCATGTACAGATACAGATAAACTTTTTCTCCATTTCTGA
GGTGCGCCATGCATAATGTAAATAATACTCAAGCGCCAACATTCTTATATAAGGCAACTTCACCATCATC
AACAGAATACAGCGAGTTAAAAAGCAAAATATCCGATATCCATAGTTCGCAAACTTCTCTAAAAACACC
AGCATCAGTGTCTGAAAAAGAAAGCTTTGCAACGTCTTTTAATCAGAAATGTCTTGATTTTTTATTTTCTT
CCTCAGGGAAAGAAGATGTGTTAAGAAGCATTTATTCCAACTCAATGAATGCGTATGCCAAAAGCGAG
ATTCTCGAATTTTCAAATGTTTTGTACTCCTTAGTACATCAAAATGGTCTTAATTTTGAAAACGAAAAGG
GACTTCAAAAAATTGTCGCACAGTATTCGGAACTAATTATAAAAGATAAATTATCCCAAGATTCTGCCTT
TGGACCATGGTCGGCAAAGAATAAGAAACTCCATCAATTACGACAAAACATTGAGCACAGACTTGCAC
TATTAGCACAACAACACACATCTGGTGAAGCTTTATCATTGGGACAAAAACTCCTCAATACTGAAGTAT
CATCATTTATCAAGAATAATATTCTTGCTGAATTAAAGTTAAGTAATGAAACTGTTTCATCTCTCAAACTA
GATGATTTAGTTGACGCACAGGCAAAACTTGCCTTTGATAGTTTGCGCAATCAACGTAAAAATACTATT
GATAGTAAAGGATTTGGTATAGGTAAACTGTCAAGAGACTTAAATACAGTAGCCGTGTTTCCTGAGCTG
TTGAGAAAAGTCCTTAATGATATTTTAGAAGATATAAAAGATTCGCATCCTATCCAAGATGGCCTCCCTA
CACCTCCCGAAGATATGCCAGATGGCGGACCAACCCCCGGAGCCAATGAGAAAACATCCCAACCTGTA
ATTCACTATCATATAAATAATGATAATAGAACTTACGATAATAGAGTTTTTGACAACAGAGTATATGACA
ATAGCTATCACGAGAACCCAGAAAATGATGCACAGTCTCCTACTTCTCAGACAAACGATCTATTATCCC
GTAACGGAAACTCATTACTAAATCCACAAAGAGCACTAGTTCAAAAAGTAACTTCCGTTCTACCACACTC
TATATCAGATACTGTCCAGACATTTGCAAATAATTCAGCTTTAGAAAAGGTTTTCAACCATACTCCAGAT
AATTCGGATGGAATAGGTTCAGACCTGTTAACTACGAGTAGTCAAGAAAGATCTGCAAATAACTCTCTT
TCTCGGGGACACAGGCCTCTGAACATACAGAACTCTTCAACCACCCCCCCTCTCCACCCGGAAGGAGTG
ACAAGCAGTAATGATAACTCATCAGATACAACTAAAAGTAGCGCTTCTCTTTCTCATAGAGTAGCTTCGC
AAATCAATAAATTCAACTCAAACACTGATTCAAAAGTACTTCAGACTGATTTTTTCTCAAGAAATGGAGA
CACATATTTAACACGGGAAACGATATTTGAAGCGTCAAAAAAAGTAACAAACTCCCTAAGTAATCTTAT
ATCTCTCATTGGAACTAAATCAGGAACACAAGAACGAGAGTTACAGGAAAAATCAAAGGACATTACAA
AATCCACAACAGAACACAGAATAAACAACAAATTAAAAGTTACAGATGCAAATACAATAAACTACGTAA
CAGAAACCAACGCAGATACAATTGATAAAAATCATGCGATCTATGAAAAGGCAAAAGAAGTATCTAGC
GCCCTCAGCAAGGTATTGTCAAAAATTGACGATACCTCTGCAGAATTACTTACAGATGATATATCTGATT
TAAAAAATAACAATGATATTACAGCTGAAAACAATAATATATATAAAGCAGCAAAAGATGTAACCACTT
CCCTATCAAAAGTATTAAAGAATATCAATAAGGATTAAAATATGATAAAAGAAAAAATATTATCAATAG
TGGCCTTTTGCTATGGTATAGCGTACAGCAAACTATCTGAAGAAACTAAATTTATTGAAGATCTTTCTGC
CGATTCTCTTTCTCTGATTGAAATGCTGGATATGATTAGTTTTGAGTTCAATCTACGTATAGATGAATCT
GCATTAGAACACATTATTACTATTGGAGATCTTATTTCTGTAGTCAAAAATAGTACAAAATCAATATAGT
AATTGATAAGCATTTTTTCATCTATGGAGCTCTCACATCAGAGCTCCACAAGAATATTATTCTTTTATCCA
ATAAAGATAAATTGCATCAATCCAGCTATTAAAATAGTAATTTTTAAGACTACCGTTGACTATCATCAAC
AGTTACATACAATCTTGCTCACACTGCATTTAACTTTTGTCAATATAAATCAATATGAATAAACAGGGTG
TGATATGGTGGATTTGTGCAACGACTTGTTAAGTATAAAGGAAGGCCAAAAGAAAGAGTTTACACTCC
ATTCTGGTAATAAAGTTTCCTTTATCAAAGCCAAGATTCCTCATAAAAGGATCCAAGATTTAACCTTCGT
CAACCAAAAAACGAATGTACGCGATCAAGAATCCCTAACAGAAGAATCATTAGCCGATATCATAAAAA
CTATAAAGCTACAACAATTCTTCCCTGTAATAGGAAGGGAGATTGATGGTAGAATTGAAATTCTGGATG
GCACTCGTAGAAGAGCATCTGCAATATATGCAGGAGCAGATCTTGAAGTTCTATATTCAAAAGAATATA
TATCTACTCTTGATGCCAGAAAACTAGCAAACGATATACAAACAGCAAAAGAGCATAGCATCCGAGAA
CTTGGTATCGGTCTTAATTTTCTGAAAGTATCAGGGATGTCCTATAAAGACATAGCCAAAAAAGAGAAT
CTGTCTCGCGCGAAAGTCACTCGTGCCTTTCAGGCAGCAAGCGTTCCACAGGAAATAATATCTCTATTTC
CAATCGCGTCAGAACTTAACTTTAATGACTACAAGATATTATTCAATTATTATAAAGGACTTGAAAAGGC
TAATGAATCTCTTAGTTCTACACTACCAATATTAAAGGAAGAAATAAAAGACCTTGATACAAATTTGCCC
CCGGACATATATAAAAAAGAAATTTTAAACATCATAAAGAAAAGCAAAAACAGAAAGCAAAACCCTTC
GCTAAAAGTTGACTCGTTATTTATTTCTAAAGACAAACGGACTTACATAAAAAGAAAAGAGAATAAAAC
AAACAGAACTCTAATATTTACATTATCTAAAATAAACAAAACAGTTCAGAGAGAAATAGATGAAGCTAT
TCGGGATATAATTTCTCGCCATCTATCGTCTTCATAAACATCGAACACTGATGTAAACTGCCCAATTCAG
ATAATAAAAATCTTCGTTGAACTTGACTTTCAGCCGGTGGGGTAATTCCGTCGGCTATCTGTTATTCAAT
CCCCACGCACTGTGTTTCCTTAAAATTACGGTGAAAACTCATACCCTGCTGACCACGAGAACATTATCTG
GTGAAATTAGTAATATTCATATCATTGAAAATGAATTGTCAACTAAGGGATGCGGCATGATGGTCAAAA
TTTTATGTGAAGATGAGTGACTGGCCGAAAATATTCAGCGTATTTTTAATTGTTTCAATAAAAACAGCG
AGGATTAGCAGTCAACGCTTTTACTACTCAATTTTTTTACATGGTGATAAACAAAAAATCCACCATTAAC
AGACTTCGTTCACAGTATCAAAAAAGCTTTAAGAATATATTAGCAGATTTCTCAAATATAATTACTGTTT
TATTCTCCTAATCTTCCCATAAAATGACCGAATTACATTATTTTATAATTATAAACAAGTTTGATTTTAAA
ATCATATTAATTATTAACAAGAGATTTATTTTTCTAACCCCAAAAGTTATGETTAGTCAAAGAGTAACTTA
TAATATCCATACCCCTCATAGGGCTTACAGTTTAAATTGCGGGTATAAAATACATCTATTTTCAAATAAA
ATAAAGCATACTCCAATTACAACGAGTGACTAAAATAAAAATATCATCCAAGTCAATCACTGTTTGCTCT
GCAAAATATGTCAGCCTTCTCAAACTTACCAGCACTTAAATATTTAAGCACACAATCATTCCTTGAGGGA
GTTCACTTATTTCAAGAGAAACATTTCGTCTTCCCACACAACTAATCCGCCCAATATAGCTAAATTTGTAC
TCAGTAATATCATTTGTTATGCCCCCCGCCCCCAAAATATTCAAGCTCCTTCACAGATAAAAAACGACAA
TTTACAAAGCCTCATTAGTTATAACTATGGAAATACCTATTTTAGTATATCCTATAAAGTTAGAATTACTT
CTTCTTGCACCTAATTCCATAGTTGAAAAACAAGAATAATTTTCTCCTACAGCTGGATCCATATAGCTTCC
ATCAGGTCTACACAACACCCAGTGGAGGCCGACAGGAACTCCAATAAAGGATACAGCAACAGCTTCCA
AAACTCTTTGGTTAGAAGATAACACATCTCGTTGATGTACTATATTACATCCCATGCCAATTAATAAGTC
CCTTGCATCGGGATAAATAAAACTAATTACTTGCGAAAAAATGTTACTTTCTTCAACGACATAAGCATTC
AATCCAAGAAGCCTCGTAGCCATGACTATCTTATCAGGCATAGAATAACCGGCATCCGCGATATTATCT
TTGTGTATTCTCGGATTATAATTACCACTAGTTATTAAGTATAAATCCCGCTCACAACGATTATCCAAATC
AAGAGAACTCTTCCTTGTCATTTCGGACATAGACCCTTTGTATTGTGGTATTTTATCAACCCCTAATTCCT
TTGCAGCACAGAGTAAACTGGCAGCTCCACAAGAATTGTCATACATCTGCTTCCTCGCCAAAAAACAAC
CTTGTTCGTTTACTCTATTCTCATTTACCCTCACATCAGTCTGATGAGATATCATATATTCTCTTGGTATAG
CCCCCTGCAATAAAACCCCATATAACATAACACCTGTATGAATACATCCCCGTTTGCAAGGCTTCCGTTG
TTCCGACATCGACGAGTAATTGCAGCACAGTATTGCATCCAGCAATGCGAACTACTATGAGAATTAGCT
GACCGGGTCCTTATTCCCCGAACGAACTTTAAATCCAAAACCTCATACTCTTTTCCGTCGTCATCACGTCC
GTCACCATAACAACCTTTCAGCATCCACCCTTTTATTAAGAGTTAATGTCCATCAGCATCAGCAATTAAT
ACCGCCTGATACTGACATATTTTTGTCGTATTTACGTCCATAATCGGTTATTGTGATTCAGGTCACACCA
GCTTATTTGCTATTGTTACAATAACCACCTTGGTTGAGAGCTGTTTTTTCAACTTTGTTATCGACTCCCCC
AACTCCTTGGTCAGTAAGGCTGCCGACCGAGCACAGTGAATAGATAGTATCCGCAGGTACCACCATGC
TTACTTAATACTCAGAAGGCGAAGTGCTCTCCAATTAACTCTGCCGCACTATGCTTATAAGCTGCTTTTC
AATGCCGTCTATCAACATGTCCAGTTCATTCAATCTGTTGTACTGGTAATCCAACGCAGTAATGATATAT
GCAGGTATTCTTTCATCCACTCCAGTAACGTTTCATGCAAAGCATTAATTTACGCGCTACGGAACTTCAC
CAGTTGACTGAGCAAACGATGTGGGACCAGTACTGCCTGCAGTTCTTCTATTGTTACAGCGATTTCTTTA
CCAAACAGCTGAACCACCATCAAGATAAAGATAGCCTGAACATTCATCGCACCATTITGTTACCAATGG
CAAAAGATTTAACGAACCGTGCCTGCATCAGGCGGACTTTTTATCCCTGCTTTGTTAGCTCCCTAACCTT
GTACAGAGAGCCTCCATAAGCTCCCATATCAAGCAGACATGGTTCGCAGTTACTGACATACACCAGAAA
ACTCTGACAGCAGACTCACTTATCCTCCACTTCACTCATATCCTTATTGGTGAGATGAACMWRMMWR
MMMWRWWWWRMRMYMWMMMMRMMMMMMRRWRWMWMWWWMMCCTGTAGAGAT
TCTACAATCTGTAGCGTTATGCATCTAATGTTAGGCTCTATGATGTCAGAAATCTGCGAGACTMWACAC
CTTCTGCATTTACCTTTATTTATCACCATGCTCCTCAGTTAACGGAGAACGTGTTCATTGCATTTTGTCAC
TTACATTTCGCCTTTGGTACCGGGGGGATATAGGTTCCAACGTTCCGCATGAGAACCTGAACCGGCTTC
ACGCCACCTTGATACCAGATGCCGCACAGGCAGTCAACAAGTGTCACGAACGGTGCAATAGTGATCCA
CACCCAACGCCTGAAATCAGATCCAGGGGGTAATCTGCTCTCCTGATTCAGGAGAGCTTATGGTCACTT
TTGAGACAGTTATGGAAATTAAAATCCTGCACAAGCAGGGAATGAGTAGCCGGGCGATTGCCAGAGA
ACTGGGGCTCTCCCGCAATACCGTTAAACGTTATTTGCAGGCAAAATCTGAGCCGCCAAAATATACGCC
GCGACCTGCTGTTGCTTCACTCCTGGATGAATACCGGGATTATATTCGTCAACGCATCGCCGATGCTCAT
CCTTACAAAATCCCGGCAACGGTAATCGCTCGCGAGATCAGAGACCAGGGATATCGTGGCGGAATGAC
CATTCTCAGGGCATTCATTCGTTCTCTCTCGGTTCCTCAGGAGCAGGAGCCTGCCGTTCGGTTCGAAACT
GAACCCGGACGACAGATGCAGGTTGACTGGGGCACTATGCGTAATGGTCGCTCACCGCTTCACGTGTT
CGTTGCTGTTCCCGGATACAGCCGAATGTTGTACATCGAATTCACTGACAATATGCGTTATGACACGCT
GGAGACCTGCCATCGTAATGCGTTCCGCTTCTTTGGTGGTGTGCCGCGCGAAGTGTTGTATGACAATAT
GAAAACTGTGGTTCTGCAACGTGACGCATATCAGACCGGTCAGCACCGGTTCCATCCTTCGCTGTGGCA
GTTCGGCAAGGAGATGGGCTTCTCTCCCCGACTGTGTCGCCCCTTCAGGGCACAGACTAAAGGTAAGG
TGGAACGGATGGTGCAGTACACCCGTAACAGTTTTTACATCCCACTAATGACTCGCCTGCGCCCGATGG
GGAGCACTGTCGATGTTGAAACAGCCAACCGCCACGGTCTGCGCTGGCTGCACGATGTCGCTAACCAA
CGAAAGCATGAAACAATCCAGGCCCGTCCCTGTGATCGCTGGCTCGAAGAGCAGCAGTCCATGCTGGC
ACTGCCTCCGGAGAAAAAAGAGTATGACGTGCATCCTGGTGAAAATCTGGTGAGCTTCGACAACCCCG
TTACTTTATTCGTACCCCTTATAATGGGGTGTTAGCCAGCCAGACCCGGCATGATTACTGCCCCCAGTCG
TCCATGATCCGGGGGGTGATGTCACCGGGTCTGGTGGGGCGCTGGTAACCGCTAATAGGGGTCAGGT
CAGGCACTTTTGCCGGGACCGTCTGTAACGTGGATGCCGGTACCTGCTCCCCGTGGTTATCTGGTTAAC
CCATATACAAGGGAGACAGAATGACCGAATCCAGCGATTACGAATCCGTCCAGGTCTTTATCGGCGTT
GATGTCGGTAAAGATACGCATCACGCTGTTGCCATTAATCGTTCAGGTAAACGCCTGTTCGATAAAGCA
TTACCCAACGACGAAAACAAACTCAGGTCGCTAATATCTGACCTGAAACAACATGGTCAGATACTGCTG
GTTGTTGATCAGCCAGCTACCATCGGTGCGTTACCTGTCGCCGTTGCCCGCTCAGAAGGAGTCCTTGTC
GGATACCTCCCTGGACTGGCCATGCGCCGCATAGCCGACTTACACGCCGGTGAAGCTAAAACTGATGC
TCGTGACGCTGCCATCATTGCCGAAGCTGCCCGTACCCTGCCTCACGCGCTACGCACGCTGAAACTGGC
TGACGAGCAAATCGCCGAACTCTCCATGCTCTGCGGCTTCGATGATGATCTTGCCGCACAGACAACGCA
GGCCAGCAACCGTATCCGCGGCCTTCTGACCCAGATACATCCGGCACTGGAGCGCGTTCTCGGTCCGA
GACTTGAGCACCCGGCGGTACTCGATCTTCTCCAGCGATATCCCTCACCAGAAAAACTCGCTTCGCTGG
GTGAGAAGAAGCTGGCAGCCCAGCTCTGCAAACTTGCGCCTCGTCTGGGTAAACGCCTTGCAGCAGAC
ATAGCTCAGGCACTGGCCGAACAAACCGTCGTCGTTCCCGGCACGAATGCCGCTGCCGTAGTACTGCC
ACGTCTGGCACTCCAGCTCATCACGCTGCGTAAGCAAAGAGACGAGGTGGCGCTTGAGGTAGAACAGC
GAGTTCTTGCTCACCCTCTTTACCCGGTCCTGACCAGTATGCCCGGAGTCGGTGTCAGGACCGCAGCCA
GACTCCTCACCGAGGTCGCCTGCCGCGCCTTCGCCTCTGCCGCACATCTCGCTGCTTATGCTGGCCTTGC
GCCGGTAACTCGGCGATCCGGCTCGTCAATACGCGGTGAGCATCCCTCGCGACGGGGTAATAAAGCTC
TCAAACGGGCGTTGTTCCTGTCGGCCTTCGCCGCGCTCAGGGATCCGCTCTCCAGGGCTTACTACACCC
GCAAAATGAGTCAGGGAAAACGACACAATCAGGCGCTTATCGCCCTGGCGAGACGACGCTGCGACGT
TCTGTTCGCCATGATGCGCGACGGGACTTTTTATACCCCGCAGGGGTCATAACATGCTTGACAACTTAA
TAGGGGCACCCCCCAAAGCTGCTGTCTCTTGATCAGAGCTCCTGATCAAGAGACTTCATCACCAGGTAA
CCCTCAACCATATCCTGAAGTCTGACCAGCCATCCCACATGACGACCCAACCGGGACGACCTGTGCGTT
TGTAGACTGGCCCCCTGAATCTCCAGACAACCAGTATCACTTAAATAAGTGATAGTCTTAATACTAGTTT
TTAGACTAGTCATTGGAGTACAGATGATTGATGTCTTAGGGCCGGAGAAACGCAGACGGCGTACCACA
CAGGAAAAGATCGCAATTGTTCAGCAGAGCTTTGAACCGGGGATGACGGTCTCCCTCGTTGCCCGGCA
ACATGGTGTAGCAGCCAGCCAGTTATTTCTCTGGCGTAAGCAATACCAGGAAGGAAGTCTTACTGCTGT
CGCCGCCGGAGAACAGGTTGTTCCTGCCTCTGAACTTGCTGCCGCCATGAAGCAGATTAAAGAACTCCA
GCGCCTGCTCGGCAAGAAAACGATGGAAAATGAACTCCTCAAAGAAGCCGTTGAATATGGACGGGCA
AAAAAGTGGATAGCGCACGCGCCCTTATTGCCCGGGGATGGGGAGTAAGCTTAGTCAGCCGTTGTCTC
CGGGTGTCGCGTGCGCAGTTGCACGTCATTCTCAGACGAACCGATGACTGGATGGATGGCCGCCGCAG
TCGTCACACTGATGATACGGATGTGCTTCTCCGTATACACCATGTTATCGGAGAGCTGCCCACGTATGG
TTATCGTCGGGTATGGGCGCTGCTTCGCAGACAGGCAGAACTTGATGGTATGCCTGCGATCAATGCCA
AACGTGTTTACCGGATCATGCGCCAGAATGCGCTGTTGCTTGAGCGAAAACCTGCTGTACCGCCATCGA
AACGGGCACATACAGGCAGAGTGGCCGTGAAAGAAAGCAATCAGCGATGGTGCTCTGACGGGTTCGA
GTTCTGCTGTGATAACGGAGAGAGACTGCGTGTCACGTTCGCGCTGGACTGCTGTGATCGTGAGGCAC
TGCACTGGGCGGTGACTACCGGCGGCTTCAACAGTGAAACAGTACAGGACGTCATGCTGGGAGCGGT
GGAACGCCGCTTCGGCAACGATCTTCCGTCGTCTCCAGTGGAGTGGCTGACGGATAATGGTTCATGCTA
CCGGGCTAATGAAACACGCCAGTTCGCCCGGATGTTGGGACTTGAACCGAAGAACACGGCGGTGCGG
AGTCCGGAGAGTAACGGAATAGCAGAGAGCTTCGTGAAAACGATAAAGCGTGACTACATCAGTATCAT
GCCCAAACCAGACGGGTTAACGGCAGCAAAGAACCTTGCAGAGGCGTTCGAGCATTATAACGAATGGC
ATCCGCATAGTGCGCTGGGTTATCGCTCGCCACGGGAATATCTGCGGCAGCGGGCTTGTAATGGGTTA
AGTGATAACAGATGTCTGGAAATATAGGGGCAAATCCAGTTWKMWRWMMWRMYMWMGMMM
MARGCCCATTTCAGGGTCGGTGCCTGCACCGGAAGCGGCTTTCCTTCCAGCTTAACCCACAGTAGTTTC
CACTCTGTCGGCGTCAGTATTTTCTCACAGCTGTCATTTTGTGTTTCTTCACTGACACCTCCCTGCCGCAG
GCCCAGCACCCGTACCGCTATAAACGCCTGGATAACCACCATGCGCTCAAGGTTATCCCGGGTCTGCAT
TCGCAGCGATTCCATGTACCACCACTTTTCCACGCCTTGTGGTATTCCTCTGTCAGCCAGCGTCGCTCGT
AATGGCTGACAATACGTCGTGCATCGGCGGCACTCGCCGCTTTTCCTGACGTCAGCAGATGCCAGCAG
GCACCGTCCTCTGCCTGCTCCCGGCAACAGACATACGTGAGCGGGAGCGCCTGGCCGCTGTTGTCGGG
ATTTTTTATGCTGACTTCGCTGTAACTGATGAACATCCGGGCCTGACGGTCTGCCAGCCGTATGCAGCC
GAACAGAGACGGTGCGATGCTACCTGTTGCTCATTACTGTAGATCCGCAGTTCATCATCCAGCGAGATC
CGTATTGATACCAGCTGGCCACACAGGCTTTCCGGCACACTGTAACGGTTCCCGCCAACCTCGATATAG
CCATCCCAGGAGACATGACGGATATCGAAGTCCGTACCCAGCAGCGGCTGCAGATGCTCCTGTTCCTGC
GTGAAGCGCTGTTCCGGTGTCTGCCTGAACTGGCGAAGTTCCCGCCTGTCGGCAACGTCAGCCATCCAC
TGCGCAGCATGAGAGTTTCCTTTGTGGTGTAGGAACCTTAAGGAAACAACATGTTGGGTGGAGCGGAC
AATCCAAATGGTGAAGGGGGGATGCGGACGATTTCCTGGACTGCTTTATCATTCCAGCCCGCATTGTTG
GCGGTACATTTTGGGGCTGACGGCACCCAGCGATAATTTTATACGCCGCTCGTTATACCATCTGATGTA
AGCATCCACTTGCTGCATGAACTTTTCTGGCGTGATGCCCGACCATTTACGCCCATAATACATTTCCGTT
TTCAGTCTGCCGAAAAAGCCTTCGCATGCAGCATTATCAGGTGAACATCCTTTACGGGACATTGAGCGA
ATAAGACCTGCTGCATTCACTCTTTCCAGCCAGCCTGGCCAGCGATAATGCCCACCTCTGTCACTGTGTA
TCACCGGTCGTTCGCCAGCATTTAACGTTTCGACAGCGCTATCCAGCATAGTGTTGACCAGTTCAGCATC
AGGGCGTGTACTGAGAGACCAGCTCACAACTTTTCCATCGAAGCAGTCCACCACTGGTGACAGCCAGA
CTTTACCTGCAGGGAGCTGGAACTCCGTGATATCTGTCAGCTATTTCTGATTAGGTTGCTCCGCCTTAAA
ATCTCTGGCGATAAGGTTATCCGGAGCCGGTCCGATTTCTCCGCAATATGAGCTGTAGCGACGACGAC
GGTTACGACTGACGACAAGCTGTTCCTCCACCATAAGTCTGCGTACAACCTTTTCTGATAGCCGCCCCCC
CTCATGCCTGAGCATCGCATGCAGGCGACGATAGCCATAACACTGGTAATTACTGTTAAAGATATCTGT
CAGCATCTTACGTATCGTGGCGTATTTATCACCGGCTTTCAGTGCAGCCCGGTGATAAAAATAACTGCT
ACGGGCAAGGCCCAGAACTGAGGTGTACTGGCAATAGCGGACACCACCATTTGTTCTTTTTCTAAGCAG
TCATCTGATAATATTTTATCCTGAAGGCTGCCGGGGAGATATTCCCCAGACGAGAGTGACGACGCTGAC
GATTGTAGAAAATCTCAATGTATTCCCGTATTACTGAGATGGCCTCATCCCGGCTCTTAAAACGATAGTG
GCTCAGGCTTTCATTTTTCAGCGTTCCCCAGAAGCTTTCCATCGGAGCGTTGTCGTAACAGTTACCTTTA
CGCGACATTGATGTTTTCAGACCAGACTGCTCCTGTATGACCCGGTAATCGTATGCGCAGWMMWRW
RMMMMTMRAWMRRTRWRRTRRATTAGMCCGGCAGGTGGRCGCTGGCTCCTGAGCGCMATAAAC
MRRRMWWWRMMYRWMMRMWMWWWYRWMMWRMRYWMWMMMMWRRMRWMRMMR
AMMMWWWMRMMMRWMWMMMMMWMWWTGACGCCGGCGAGATACAGCCATCCTTCCCG
GGTGGCAACATACGTTATATCTGCGACCCAGACCTGATTAGGTGCTGTGGGCGTAAAGTTCTGATTCAG
CAGATTCGGCGTAACCGGCAGATTGTGGTCAGAGTTTGTGGTCGCCCTGAACTTGCGTTTCTGCTTACA
GTGCAGCCTCAGCTCCTTACGAAGACGTGCCAGTCTGTCACGACCGACGATAATTCCGTTATCTGCCAG
CTCCGTCTGGAGCCGCCGGGTTCCATATGTTTCGCGAGTGCGGATATGTGCCACCTTAATCTCCAGTTTT
AGCCGCTCATCACTTTGTTTTCTGTCTGAGGGGACATGCTGTACCCGGTCGTAATAACCGCTCCTGGATA
CACCAGGTAMSMRMMAMMWMRMWWMMMMMRRRMMKWRWWRWMRMMMWWRWWC
GACTAACGCGTATTTTTCAGCGACTCCTGGGCAAAATACGCTGTTGCTTTTTTTAATATATCTCGCTCAA
GTCGTGCTTCATTTAAAGCCTTACGCAGTTGCATAACTTCAGATTCCAGCTCAGCCACTGTGCGGGAGC
CTGGTGTATTGAGCCCTTTTCTTGCGGCGGTAACCCATTGTCCTAAAGTGCCTTCAGGAACAGATAATC
GGGAAGCGCCTTCACTGATCGAAAGCTGATTTTCAGGAACCGTTTTGACGGCTTCGGCTTTAAACTCCG
TAGAGTAACGTTGGGTTTTTCTGCTCATTATTGGCTCCTTCTGATGCCATTCTATTTCAGGAAGGAGTGT
CCGTTAAACTCAGGCTACCTCACTCCCGTAAAGACGGGAGTCAAGTTCCCGCTCCGACATAACTGCGTG
CAATGGATTTTGAAGGAACCGTCAGCCCATTCCCCGGAGGCAAACATGGGCTGGCAAAAGTGTAGCGG
TATTAGGCACAGCTATTTAGGAAAGAGGGACCATCTCCATCTCATTAAATGGACCACGCCAGACAATTC
ACTGTCATGCTGATGCTGCACTGTGGGGACAGAATCACTCATGATGACTTCGATAATCGACGACATTAT
TATTTGGCCGAGCTTTTAGGTCATATCTCAAATCGTTTGCCTAATATTGCTCCATTTTTTAATAAAAAATC
AATCATTTTTGAATCTTTGCTTTTCATTGCATTGTCAAGCATAGTGTCCCCACTATTTGCTTTTTGAAATTT
TTTGTTTACATCAACCAATCCATTATTGATAAAATATTCTAGCACTTTATAGTTGGCACCATCTTTACTAA
GTAAGTATTCAACATCATACAGATTACAATATCTATTGTTGTTCATTTTCTCCATCTCTGCGACATCTTGT
TTTGTAAAATTAAATTTAGATAGTAAATAAAGTGCTATATCAGCTTTTTTATTGCCTAAAGAATAAGCAA
GGGCCTGATGAGCCATTTTTTTATCAGTCACTTTATTGTTAATCTCTTCATAATTAGATGCTTTAATCGCA
TCCTCCAAAGATATGTCTCTGAGTTTTATTTTTTTGAAGTTATCTGTACTTAGCATTCTTGGTATATGATA
CTCAAGCTGAAACACAAAGTTTATAATTCTATCCAGACTAACAGGATCAATATTTTTGTTATAACAAAAC
TCTCTGAATCCTTGATCTTTACTCTTTCTTATAAAATCAATAAGATGCAACCCAATTCCAAGCCTCATATC
TTTAGTGTTAAATATTGGAACATCAGTTTTCCCCTGATTACCATGCACATACCTGCTAACTTCATCCACAA
CCTCTTTAAAGTTATCTAAGAAAAATGATTGGTGCTCATGGTAAAAAACAGGTGGAATAGCATTATCAA
AGTGATCGGTTAATGTCATATATCCATATGGAGAGTCATGATCAATGATATACGCATTGGCGCCAAAAT
CTACAGTATGATGTGTTTTATTTAGTGGGAGTTTTTCTTGATGATTGGAAATTTCAACCCCAAAAAAAAC
AAAATCGTGGTTAGAGATTTCTTTAATATCCTCAGAGTATGTATTCTTATGTGTTTGTATATTTTCAGACT
GAATTTTATCAAAGGATTTAATATTAAGAATATTATTTGACAGATTAGAGTTACTTTGATGTCTGAAGTT
AAAATTAGAATTAATAATAATAGAAAGTAAGTGACGTTCGTCTGAAGAAAGAGGATGAACTTTTTGTAT
TATCTCAGAGATATAGTGTCTGGACACCTTATCGAGATGCTCAAGTGACCGAAATGATGACCGTGCTCC
AGATGGCGCTTCAATAGAAAGATTTAGTGATTTGAAACTGGTCCCTTGAGAAAACATTTTGCTATAACT
TTGCGCTGCTATAGACTTCCTTAAAAAGTTTTTCACAGTATTAGCACAGTGATCTAAATGTTTTTCACTTA
TGCCAGAAGAAGATGAGTTTGTTACGCGTAATATATTTTTTTGTAATGCTTTTGTTGCTTTATTCGGATTC
GTTTTTCCATCAACAAGATCAATATTATTCTGAACATTAATATGATTTACTGCTTCAGGTATTTTCATCCC
CCCCCCAATTATGTGCGTTTTTATCTGTGGTTATTTAGATAACATTATCTAACTGGACACTTTCTTGTATG
TATTATAGCTAAATGAACAGATTTTAATATGTTGTATGGTTTTCACTCTGAAATCCCCTGAATTTAATCAC
GTTTCCTTTACTGAAGGCAATTTTAACCGTAGTATCAATTATTAACCCTTGGGCTTGATATCCCTCTCCTC
TTCTGGGCACGGGGGATGTCAGATTACTTATTACAGAAACCAAAAGAATTGTAAGCGTCATCTCAGTGT
CGTCTATTTCAATGTCTGATGATTGGCGATGATAGAGTCCTCTTATAAGTAAGCGCCCCATCAGCGACG
TCTTGTGAAAATTGTCCTGTCTGGCAACAATCGCGCCCATCTATATTGATGGACACGAACGATGAATTCC
CAGACAACAAAAGATATTCCCTGCTTCCGTTCTTATTTGCCTGATGCCCTGCGTTTAAGATTTGAAGATA
AACTGACCATCCGGGCCATCGCTCAGCGTCTGGGTCTCAGTCATTCCACAATACATACGCTTTTTCAGCG
ATTTCTTGCATCCGGTATCGCATGGCCATTGCCCGATTCAGTTTCATTCGCTCAACTTGACGCCATCCTTT
ATGCCAACAGAAAGAAGGAATTAACAGAGCCTCAAATCAGAGAAGGCTCATGGCGAAAAGAACGGCG
AACCAGCTATAGCCGTGAATTTAAGGTCCGTCTGGCTAAGCAGGCATTACAGCCTGGTGCTGTTGTTGC
CCGGATCGCCAGAGAACACGATATCAATGATAACCTGCTGTTTAAATGGAAAAGCCAGTACGAGGACG
GCTTACTGAGCGATGATGACATACAGGAATGCATGCCTGTCCCGGTGGCACTGACTGATACGCCGGAG
CCGACCAGACCAGTTACAAATCCCTTCTGGCGTAAGAACCACGGCAGCCTGGCGGCTGCAAATCGGGG
TGTGGCAGAATATGAGCTTTCAGAGTAACCATCCAGAATGAGTTCCCATGTCACACAACCTGGAGCACC
AGAAAGTCCACACCAGAATGGTGAAAGAGGTTCTGAAAGCCGTCGCCAGAGCCAATAATCACCCTTAT
CAGTCCGTATTTACAGACTTTATCGCCGGTCATCCCTCCTGCACGGTCTGTTTCTGGGAAACCTTCCACA
AAATGTACCCGGATTCACCGTACGAATACGTCACGTTCTGCCATACCTGCCGTCGTTTTGATTTATACGA
AACAGAAGCGGAGATGAAGGCGGATGACCCGAAGTGGTGGTGATTTTCAGCCCCGACCGCTGAAACG
TCTGTTCACGGCCAACCAGTGCTGGACATCCTTCCTGGATGCGGGCGGTCTGCGCGATATCGAGGTTG
AGGCTGTCACAAAAATGCTGGCCTGCGGCACGCGGATACTGGGAGTGAAGGAATACAACTGCGATAA
ACCTGAGTGCCCCCACGTCAGATACGTCACTAACTCATGCGGCAGCCGTGCCTGCCCGTCCTGCGGAAA
AAAGGCCACAGACCTGTGGATAGCGACACAGCTGAATCGTCTTCCTGACTGCGACTGGGTACATCTGG
TCTTCACCCTGCCGGACACGCTGTGGCCGGTGTTCGAAAGCAACCGGTGGCTGCTGAATGACGTGTGC
CGTCTGGCGGTGGAGAATCTGCTGTATGCCGCCCGGAAACGGGGGCAGGAACCCGGTATCTTCTGCGC
CATCCACACGTATGGCCGTCGTCTCAACTGGCATCCGCATGTACATGTGTCTGTAACCTGTGGATGTCT
GAATAAGCATGGTCAGTGGAAAAAGCTGAGCTTCCTGAAAGACGCGATGCGTTCGCGGTGGATGTGG
AATATGCGGCAACGGCTTCTGAAAGCGTGGTCAGAGGGGCTGGCGATGCCGGAGTCGTTGTCACATAT
CACGACGGAATCACAGTGGAGAAGCCTGGTGCTGAAAGCCGGAGGAAAATACTGGCATGTGTACATG
TCGAAAAAAACGGCCGGCGGGAGGAATACGGCGCGCTACCTGGGTCGTTATCTGAAGAAGCCGCCGA
TAGCGGCCTCCCGGCTGGCACATTACAACGGAGGGGCGAGCCTGAGCTTCCGTTACCTGGACCACAAA
ACGGGAGAAACGGCGACGGAAACATGCACAAATGGTGAAACAGTTCCTGAGTCGTGACCCGTTCGAA
TGCGTGCTGTGTGGCGGCCGGATGGTATACCGCCGGGCCATTGCGGGGCTGAATGTGGAGGGGCTGA
AGAAAAACGCGCGGGATATCAGTCTGCTGAGGTATATGCCGGCCTGACACAGGCTAAGTGTGCCCGG
GAATCGGTGCAAGTATAAAAAACAGCCTGTTRCNNNNNNNNNNNNNNNNNNNNNNNNNNNNNNN
NNNNNNNNNNNNNNNNNNNNNCAGAAAGGCTCACGTGGCATAAGGCGCGGACAGGGGTATCGCT
TAGTTTTTCAATTTCCTATACGTCAAGCTGTGCACGTCCATCCGATGCCATTTTCTCATTTGACTTTCGGG
GGATTACTGCCTTTATACCGTTATTTTTCAGCTCATTGCGCAATGCGCGCCCTGAATACGCTTTATCAGC
CAGTACCGCATGTCCACGGCGTTTCATGCTGCCGTTCTGACGCTGAACGCCAATCCCGTCCAGAAGTCG
TTGCGCGAACTGGCTTTCATGAGCTTGTCCGGGGCTTAGCACGATATTTAACGGGAGGCCACTTCCGTC
TGTTGCCAGATGGATTTTGGTGCCAAAACCGCCGCGAGAGCGACCCGGCCCATTATCTCCGTCGATATC
GGGATGTTTTTTTGAGCACCGGCGGCACATTTCAGCGCMMKRMWMWWMMYRMMMWMMMRM
RMRGTRRCARACMARTSAAWMMMRMMRWWWRMMYMMMGRRTRMYKAYTCCARCTTATTGAT
AGTGTTTTATGTTCAGATAATGCCCGATGACTTTGTCATGCAGCTCCACCGATTTTGAGAACGACAGCG
ACTTCCGTCCCAGCCGTGCCAGGTGCTGCCTCAGATTCAGGTTATGCCGCTCAATTCGCTGCGTATATCG
CTTGCTGATTACGTGCAGCTTTCCCTTCAGGCGGGATTCATACAGCGGCCAGCCATCCGTCATCCATATC
ACCACGTCAAAGGGTGACAGCAGGCTCATAAGACGCCCCAGCGTCGCCATAGTGCGTTCACCGAATAC
GTGCGCAACAACCGTCTTCCGGAGACTGTCATACGCGTAAAACAGCCAGCGCTGGCGCGATTTAGCCC
CGACATAGCCCCACTGTTCGTCCATTTCCGCGCAGACGATGACGTCACTGCCCGGCTGTATGCGCGAGG
TTACCGACTGCGGCCTGAGTTTTTTAAGTGACGTAAAATCGTGTTGAGGCCAACGCCCATAATGCGTGC
AGTTGCCCGGCATCCAACGCCATTCATGGCCATATCAATGATTTTCTGGTGCGTACCGGGTTGAGAAGC
GGTGTAAGTGAACTGCAGTTGCCATGTTTTACGGCAGTGAGAGCAGAGATAGCGCTGATGTCCGGCAG
TGCTTTTGCCGTTACGCACCACCCCGTCAGTAGCTGAACAGGAGGGACAGCTGATAGAAACAGAAGCC
ACTGGAGCACCTCAAAAACACCATCATACACTAAATCAGTAAGTTGGCAGCATCACCTGCWKYMMRW
MRMRMMRRMMMMMWRWWRMMMMWMMWMYWMMWMMSRMMWRMMWWWRMM
MMWMRRWWMRMRMRRWWMWMMMMMRWTWTCCMTGACCCATATCGTTCGGGTAAATCGC
GCCATGGTGCACCGGAACATAACACCCAGAACATGCCATTGATGATTTTACGGTGCTCAGCCCATGGGC
GTCCGGCCCGTGGTGTGGCGGGTTCAGGAGGTAACAGGGGCTTGATGATGGTCCATGCCTCATCGGG
AAGGTCGTAGCAAGCCATAGTTCAATATGTTGTGTAAACAGACAGTTACTATAACTCAGATGATTAAGG
GACACAGCCTACTATTCCGCGCAGCTATTTAGCTCTCTTTTTGTTGAGCGCCCATGATTCTTTCATTTATT
GTTACTAATTTTTTATGTTATCATCATGTTATGAGCATGCCCTGTTCTGCGTCTCTATTTTATCTATCATAT
TCTGAGCATTAACTACATAACAAGGCGCAAGATATATATCCAAAAACAAATAATACTGTTGCAATAATT
CCTTTCAGTAAAGGAAGTGTGATTAAACTCAGGGGATTTCAGAGTGAAAATCGTACAACATATTAAAAT
ATGTTCGTTATAATACATACAAGAAAATATTAAGTTAGATAATATTATTCTAAATAACTGCAGATAAAAA
CGCACATAATGGGGGATGTITTATGAAAATACCTGAAGCAGTAAATCATATTAATGTTCAGAACAATAT
TGATCTTGTTGATGGAAAAATAAATCCGAACAAAGATACAAAAGCATTACAAAAAAACATATCATGCGT
AACAAACTCATCCTCTTCTGGCATAAGTGAAAAACATTTAGATCACTGTGCTGATACTGTGAAAAGCTTT
TTAAGGAAGTCTATAGCAGCGCAAAGTTATAGTAAAATGTTCTCTCAAGGGACCAGTTTCAAATCATTA
AATCTTTCTATTGAAGCGCCATCTGGAGCACGGTCATCATTTCGGTCACTTGAGCACCTCGATAAGGTGT
CCAGACACTATCTTTCTGAAATAATACAAAAAACTCATCCTCTTTCTTCGGATGAACGTCACTTGCTCTCT
ATTATTATTAATTCTGATTTTAACTTCAGACATCAAAGTAACGCTAACCTGTCAAATAATACTCTTAATAT
TAAGTCCTTTGATAAAATTAAGTCTGAGAATATACAAACATATAAAAACACATTCTCTGAGGATATTGAA
GAAATAGCTAACCACGATTTTGTTTTTTTTGGGGTTGAAATTTCCAATCATCAAGAAACACTCCCACTAA
ATAAAACACATCATACTGTAGATTTTGGTGCTAATGCGTATATCATTGATCATGACTCTCCATATGGATA
TATGACATTAACCGACCACTTTGATAATGCTATTCCACCTGTTTTTTACCATGAGCACCAATCATTTTTCTT
AGATAACTTTAAAGAGGTTGTGGATGAAGTTAGCAGGTATGTGCATGGTAATCAGGGAAAAACTGATG
TGCCAATATTTAATACTAAAGATATGAGGCTTGGAATTGGGTTGCATCTTATTGATTTTATAAGAAAGA
GTAAAGATCAAAGATTCCGAGAGTTTTGTTATAATAAAAACATTGATCCTGTTAGTCTGGATAGAATTA
TAAACTTTGTGTTTCAGCTTGAGTATCATATACCAAGAATGCTAAGCACAGATAACTTCAAAAAGATAA
GACTCAGAGATATATCTTTGGAGGATGCCATTAAAGCATCCAATTATGAAGAAATTAACAATAAGGTGA
CTGATAAAAAAATGGCTCATCAAGCTCTTGCTTATTCTCTAGGTAATGCAAAATCTGATATGGCACTTTA
TTTACTATCTAAATTTAATTTTACAAAACAAGATGTCGCAGAGATGGAGAAAATGAACAACAATATGTA
TTGTGAGCTGTATGATGTTGAGTATTTACTTAGTGAAGATAGTGCCAACTATAAAGTGCTAGAATATTTT
ATTAATAATGGATTGGTTGATGTAAACAAAAGATTTCAAAAAGCAAATAGTGGGGACACTATGCTTGA
CAATGCAATGAAAAGCAAAGATTCAAAAATGATTGATTTTTTATTAAAAAATGGAGCGGTATCAGGCAA
ACGATTTGGGAGGTGACCTAAAATATATTTAGGTTGTGCCCTGAATCTGACGTTGTCATTTTTGTTATAT
TGATCGATATAGGGGATGGTGCAGACCGTTCCCCTGCAACAACACATTGCATGTGGTGAACGCCCCGG
GAATCCTGGAGACTAGGCTGTGTCCCTTAATTACGTAAGCGTTGATAAAAGAGTCGGATGCAGCCCAG
TTTCACCATCGCCAGGTAATTTCTCGCCGTTTTGTCGTAACGCGTGGCGATGCGGCGATATTCTTTCAGC
CGCCCAAAGCACCGTTCAACGACATTACGATTGCAGTACGCATCACGATCAAGCTGTGCACGTCCATCC
GATGCCATTTTCTCATTTGACTTTCGGGGGATTACTGCCTTTATACCGTTATTTTTCAGCTCGTTGCGCAA
TGCGCGCCCTGAATACGCTTTATCAGCCAGTACCGCATGTCCACGGCGTTTCATGCTGCCGTTCTGACG
CTGAACGCCAATCCCGTCCAGAAGTCGTTGCGCGAACTGGCTTTCATGAGCTTGTCCGGGGCTTAGCAC
GATATTTAACGGGAGGCCACTTCCGTCTGTTGCCAGATGGATTTTGGTGCCAAAACCGCCGCGAGAGC
GACCCGGCCCATTATCTCCGTCGATATCGGGATGTTTTTTTGAGCACCGGCGGCACATTTCAGCGCCCG
GATATTACTGCCATCCAGCGCGGTGGCAGACCAGTCAATAAAGCCGTTTGCATCAAGTAGCGAAAGCA
ACCTGTTGAAAATAATATTAATCACGCCTGACTTTGACCATCGGTTAAAGCGGTTATAAACAGTTTTCCA
TGACCCATATCGTTCGGGTAAATCGCGCCATGGTGCACCGGAACATAACACCCAGAACATGCCATTGAT
GATTTTACGGTGCTCAGCCCATGGGCGTCCGGCCCGTGGTGTGGCGGGTTCAGGAGGTAACAGAGGC
TTGATGATGGTCCATGCCTCATCGGGAAGGTCGTAGCGAGCCATAGTTCACGTATAGGAAATTGAAAA
ACTAAGCGATACCCCTGTCCGCGCCTTATGCCACGTGAGCCTTTCTGTTTTTCCCCACCACTCCGGTGTTT
TCTCTCTCTGCATGGTCGATTCTTATACCGCAACAGGCTGTTTTTTATACTTGCACCGATTCCCGGGCACA
CTTAGCCTGTGTCAGGCCGGCATATACCTCAGCAGACTGATATCCCGCGCGTTTTTCTTCAGCCCCTCCA
CATTCAGCCCCGCAATGGCCCGGCGGTATACCATCCGGCCGCCACACAGCACGCATTCGAACGGGTCA
CGACTCAGGAACTGTTTCACCATTTGTGCATNNTTCCGTCGCCGTTTCTCCCGTTTTGTGGTCCAGGTAA
CGGAAGCTCAGGCTCGCCCCTCCGTTGTAATGTGCCAGCCGGGAGGCCGCTATCGGCGGCTTCTTCAG
ATAACGACCCAGGTAGCGCGCCGTATTCCTCCCGCCGGCCGTTTTTTTCGACATGTACACATGCCAGTAT
TTTCCTCCGGCTTTCAGCACCAGGCTTCTCCACTGTGATTCCGTCGTGATATGTGACAACGACTCCGGCA
TCGCCAGCCCCTCTGACCACGCTTTCAGAAGCCGTTGCCGCATATTCCACATCCACCGCGAACGCATCGC
GTCTTTCAGGAAGCTCAGCTTTTTCCACTGACCATGCTTATTCAGACATCCACAGGTTACAGACACATGT
ACATGCGGATGCCAGTTGAGACGACGGCCATACGTGTGGATGGCGCAGAAGATACCGGGTTCCTGCCC
CCGTTTCCGGGCGGCATACAGCAGATTCTCCACCGCCAGACGGCACACGTCATTCAGCAGCCACCGGTT
GCTTTCGAACACCGGCCACAGCGTGTCCGGCAGGGTGAAGACCAGATGTACCCAGTCGCAGTCAGGAA
GACGATTCAGCTGTGTCGCTATCCACAGGTCTGTGGCCTTTTTTCCGCAGGACGGGCAGGCACGGCTGC
CGCATGAGTTAGTGACGTATCTGACGTGGGGGCACTCAGGTTTATCGCAGTTGTATTCCTTCACTCCCA
GTATCCGCGTGCCGCAGGCCAGCATTTTTGTGACAGCCTCAACCTCGATATCGCGCAGACCGCCCGCAT
CCAGGAAGGACGTCCARMMMWRRWWRRYMRWRMMMRRMMRWWWMMRMRRKMKRRRAGT
AAAAGCGGACAACATGAGGACGGATTAACCCCCGGTATCCTGTAGCCGGGTAAGGGCTGCTGTGTCGC
CACGGCGCTCCAGGATGGAGGCCGCGACCATCGCAGGTGGTGGGTCCATGGGTTTGAGCATCTTCATC
CAGTCCCATTCACGTTGTGAGAGCTTCTCAGCACCAACAGAGAGCAGAACGGTATCTATACTTTTGCGG
GTCAACATAAGAGGATGAGTCCTTAATAAAACAGGTCCTCAGAGCATACATATTCTGCGGAGCGTGCG
GCAACAACAGGACGAACAGGGGCGGTTTTCAATTTGCAGCCGTCAGGCTGCCGTGGTTCAATATGTTG
TGTAAACAGACAGTTACTATAACTCAGATGATTAAGGGACACAGCCTGGAACCCAGGATGTTGCAGGG
AAGAGAACTTCATTCAAGCTTCATCATGAGAAGCCGATCAGCCAAAATGGTGGTGTCTATGATATGGAT
AACATCAGCGTGGTAACACCTAAAAGGCATATTGATATTCACCGAGGTGAACCGCCCCGGGTTTCCTGG
AGAGTGTTTTATCTGTGAACTCAGGCTGCCAGATCATCGTTTCCGATGGAAGCATAATAAGCTTTTTCTG
CTTCTGCCGGAGGAGTATGGCCCAGCCTTTCCAGCAATCGTCGATTGTTATACCAGTCCACCCACGTGA
GTGTGGCCAGTTCCACTTCTGCACGGTTTTTCCAGCTCTTACGGTGTATTACCTTCGCTTTGTAAAGACC
ATTGATGCTCTCCGCCATCGCGTTGTCATACGAGTCGCCTGTACTTCCTGTTGATGCCAGTAATCCGGCT
TCCTTAAGCCGCTGTGTGTAGGCCAGCGATACATACTGAGAACCTTTATCACTGTGATGGACCGTGCCG
GACGGTCGACGGGCCCATAACGCCTGCTCCAGTGCATCCAGCACGAATGTCGTTTCCATGGACGATGA
GACCCGCCACCCCACGATGTATCCGGCAAACACATCAATGATGAACGCCACATAGACGAAGCCCTGCC
ATGTGCTGACGTAAGTAAAATCAGCCACCCACAGCTGGTCAGGTCGTTCTGCCACGAACTGACGGTTTA
CGCGGTCGCCTGCGGCAACGGCTTTCCGGCTGATGGTCGTACGGACCTTTTTACCCCGGAGAACACCG
GCAAGTCCCATAACCGCCATGAGACGTGCCACAGTGCATCTGGCCACTCTGATACCTTCCCGTAACAAC
TGACGCCAGACTTTACGCACACCGTATACCTTGTGATTTTCATCGTATACGCGCTGTATCTCTTTCTTCAG
CCAGTCATCGCGCTGCGCACGGGCACTGCGTTTATCCGGATGATGTCGCTGTTGCTGACAGTGGTAATA
CGTTGACGGGGCAATATGCAGTTCGCTGCATAGCGGTCCGACCCCGTACTGCTCACGCAGCTTATCCAG
CAGTGGCATCATTTTTTCCAGAGGCGGTCAAACTCCGCCTTCGCAAAATAAGCGGAAGCCTGGCGAAG
GATATCGTTACTGCGGCGCAGTTCACGATTTTCACGCTCCAGCTCTTTCAGACGCTGACGTTCAGCGGT
GGTGAGCCCTCCATCACCGCCCCCGGTATCCCGCTCATGCTGGCGAACCCGGACACGCAGAGTCTCCG
GCGTACAGCCAATCTTTGGAGCAATGGAACAAATTGTCGCCCATTGTGAGTCATATTCGCCCTGACTTTC
CAGAACCATACGGACTGCCCGTTGACGGACTTCGGGGGAAAAACGAGTATTTTTAGTCATCCTGTTTAC
CTCTTTCTCAGGRAGTTTAGTCTCCAGGATTCCCGGGGCGGTTCAGGCCAGTACCTCGTCAGTCAACTG
ACGGTAAATCTGCTGTTCAGTCTCACGCATCACCTGTGCACCGGCTTCCCTCTCCGCATCCGCATCACCG
CTCAGACCTGATGCTTTCAGCCGGTCAGCCACCCTCTGAGAGTACTCATTCTCCAGCATCTCATACTTCT
GCTCTTCTGCCTGCGCCCAGCGGTCAGCTTCCGTACGCTTCAGTACAGCATGCCATGGTCCCCAGAGGG
AGAACCAGTCCGTAAATTCATTCTCTTCACGGCTTCTGACCATGGCTTCGGCAGTGCGGAGGTCATTTG
CTGTCACTCCCGACACGCCATAGAAACGCATTTCCTTCACGGCAGTGGAGAGCTGAAGTTTCTCTGCGA
GCATGGTCTGGAAGGCCAGGTAGACTTCTATCTCATCCACAAAATGGAGAGTTCTGACTTTATCCCGGG
CAATGTCCTCCAGAATTTCGAGGCGGAACATTTCCCTGCCCAGGGAGAGCAGAGCGCCGGTATCATTAT
CGAAAAGGCCTTCTGATGCCTGATGGACCAGGAGGGTTTTCCGGAGATTGTTCCATGTGAGCGCGACA
CGGTCCTCACAGCTCTCAGTGGCATCAGCAGCAACAGCGAAAGACTGCTGTCGAAGCTCCGCAGAGGC
ACTGAGTTTTTCCAGCCATGCAGCGACCTGTTCACGGAATCCGGAGGTATTGCGTGCAGAGACGGTATC
GGAAAGGCGGTCAAGGAACGCGGAAAAGGTGTTGGCATGCTCTTCATGTTCAAAAGCATGCCATATCT
GTGATACATCAGATTGTTTGTTTTCCGGGAACCATGCTGTCACGGCATCAGCCAGGGGGCGATGGAGT
GTATTCTGTTGTCCGTCACTCATGGAGAAGTAAATCTGCGGGCCGTGGTAGTCCGGCGAAGAGGTTAA
TCTTTGCAGGGATTGCAGAACGCGAGTCGACAATGGATTCCCTGAAATGTTAACATTCGTACAAATGGG
CAATGTGATTATATTTTCGGGTAATGAAGTTAGTAGGTTATTGGAAACATCCAGTGCCAATAGTTCTTGC
GGTAATCTGGATAATCTGCGTAGTTGATTTTCACCTACCTTCAAATATTTTAAGCGCTGAGGAAATTCTG
GTAGTTCCTGTAGTCTGTTGCCGGAAACAAAGAGTTCTTGTAGTCTATGAGGCAATTCAGGCAAGACAG
TAAGGCGATTACCTTCAATACGTAATATTGTTAGCGTTGATGGAAGCTCTGGTAACGTCTCCAGACTATT
AAAACGTGCCGAGAGTGATTGTAAATATGGTGGTAATGATGGCAGACATGACAAATTTCGATTGAATG
AAATATTAAGCTTAATCAGTTGAGATGGAAGTGTCGGGAGTATATTCAAGTTGTTACCATTTACATGAA
GTTCTGTCAAAAGCGGAGAATTTTCAGGTAGGGAGATTAACTCATTGTTGGAAATATTAAGTTCACGTA
TATGCAAAGGGAGTGGTGGTAAAGAACGTAGTTTCAGTAAGCTTAAATTGAGAACAGGCTCCCGGGTA
TCCAGACATATTTTCAATAATCGAACCGCCTGGATGCGATTTTCTGTTGTTGTTCCTTCCCTAGCCCAGTC
ATCCCAGATGCGGTGGTAATCTCTGGTACACTGTTCTTCTACCGATAAACGAGATATACATGATAAGCC
ACAAAGGGAACGAAAAAAAGAATGATTGTTGATCGGTTTCATTTATTAACCCCTCACTATTCATATCTTG
GTCTATTAATGCAAATAAAACGAGAAAGTCTTTAAAAAAACAATCCTAGAGCTTTATCGGCGTTTTCATT
CCAGCTGGAGGAATATGTTCCTGTCTTTCCAGTAACCGTCGATTGTTATATCAGTCTACCCATGTTAATG
TGACCAGTTCTACTTTCATCCAGTTTGGATTGATCTTTATCCCCTATATTTTCAAACAATTTTTATCATTTA
ACTCATTACTGTCTCGATATTCCCATAGCAAACGATCTGGTTGGTTAGCCCCTCAAAAAAGTCAGTTGCA
CGCCGTAAATCCCTGCCGGTATTGAGCGAGGATTTACGGGTTGTTGTTAAGGTCCGTAATCGGGTCACA
RWWWWTCCGGAGTCARWCMRGTCTRYGGTTTATGCTTATGCGACGTGATTATGAATGGTGCAGTCG
TGAGCCGTTTTCAGACAATCGCAGGGCCAGTACCTCGTCAGTCAACTGACGGTAAATCTGCTGTTCAGT
CTCACGCATCACCTGTGCACCGGCTTCCCTCTCCGCATCCGCATCACCGCTCAGACCTGATGCTTTCAGC
CGGTCAGCCACCCTCTGAGRGTACTCATTCTCCAGCATCTCATACTTCTGCTCTTCTGCCTGCGCCCAGC
GGTCAGCTTCCGTACGCTTCAGTACAGCATGCCATGGTCCCCAGAGGGAGAACCAGTCCGTAAATTCAT
TCTCTTCACGGCTTCTGACCATGGCTTCGGCAGTGCGGAGGTCATTTGCTGTCACTCCCGACACGCCATA
GAAACGCATTTCCTTCACGGCAGTGGAGAGCTGAAGTTTCTCTGCGAGCATGGTCTGGAAGGCCAGGT
AGACTTCTATCTCATCCACAAAATGGAGAGTTCTGACTTTATCCCGGGCAATGTCCTCCAGAATTTCGAG
GCGGAACATTTCCCTGCCCAGGGAGAGCAGAGCGCCGGTATCATTATCGAAAAGGCCTTCTGATGCCT
GATGGACCAGGAGGGTTTTCCGGAGATTGTTCCATGTGAGCGCGACACGGTCCTCACAGCTCTCAGTG
GCATCAGCAGCAACAGCGAAAGACTGCTGTCGAAGCTCCGCAGAGGCACTGAGTTTTTCCAGCCATGC
AGCGACCTGTTCACGGAATCCGGAGGTATTGCGTGCAGAGACGGTATCGGAAAGGCGGTCAAGGAAC
GCGGAAAAGGTGTTGGCATGCTCTTCATGTTCAAAAGCATGCCATATCTGTGATACATCAGATTGTTTG
TTTTCCGGGAACCATGCTGTCACGGCATCAGCCAGGGGGCGATGGAGTGTATTCTGTTGTCCGTCACTC
ATGGAGAAGTAAATCTGCGGGCCGTGGTAGTCCGGCGAAGAGGTTAATCTTTGCAGGGATTGCAGAA
CGCGAGTCGACAATGGATTCCCTGAAATATTAATCCTGTAACTTGAATCTAATTGTGAAAAACTCTCCG
GAAGAGTGGTTATCTGATTAAAATGAAAATAATATTCCTTTTGTCTGATATTGTTTCTATCAGAAAATTG
AGGAAAAGTAACCAGATTATTGCGCTGAGCACAGAGAATTTCCAGTTTATCCGGTAAGTCGGGTAATG
TTGTCAGCCTGTTATAATAAACATGTAAAAATTTCAATTTTTCGGGAAGAGCCGGTAAGTTATATAGCCT
GTTATTTTCAACAAATAATGATGTTAAATGGTTTGGCAAAACAGGAAGGTTTTCCAGTTGATTAGAACG
AACACTTAAAGTTGTTAATGACTCAGGAAGTTCTGGTATCACAGAAAGCCTGTTATTATCAGCATTAAG
TTCTTTGAGAAATGCAGGCAGCTTTGGCAATGATGTTAATTTATTATTTTCTACAATCAAAGCAGAAATA
TGCTTTGGTAATTCGGGAAGAGACGTCAAATTTAGGTCTGATAAATTTAATACTGCTTCTTGATTCTGAA
AGCACTGGGAGAGTCTGTTAAAAGCAATGCCTCGCTCTTCCCCGGGAGAAGAGTTCTTTTCCCATTCAG
TCAGGATTCTCAGATAATATTCATTACTGGTTGAGTTTGAGTTAATAGAGGGGGAGCAAGAAACTGAT
GAGTGTGTATTATTTACAGAGAACATAATGETAGGTCAACCTTATATTTGTGAGAATAAATATTTAAAAT
TGTATATTTATACAAATTTCACTTACCATTTCCGATATTAAGTTTTAATAGTTTCCATTTTACACATAACAG
AAGTTAGTTGTAATTTATTTATTGAGGTTTTCTATGGAATTCTTTTTTTATTGTGACTGCAGTCAATAATA
ACACTGAATTCAACATTCCAGTGAGCAAGGAAATGTAATCCGAGTAAGTATATTTGTTTTACATCATATT
TTAGCCAAGCTCATTTATTCCTATTATAGAATGATAAATTAAAATTACTGTAAAAAAACGATACACATTT
AATACGAGGGAGGCCGGCAGAAAAACGGCTTGCGGTAAGGCAGGAGAAATGTGTGCCGCGAATGAA
GTCCTTGTATGACTGGATAAAGCTCCAGAGGAAAACTTTGTCAAACCATGCTGAAATGGTAAAGGCGTT
CAACTATATCCTGAATCACTGGAATGCACTGAATGAGTTTTGCCGGGTAGTACTTCCAATCATTAGAATA
TGTGCTTTTCAATGAAGGAACCGATGACTTTTTCGTGGATTTCCACGGAACGCGAGAAGCAGATTGTTT
TACGTGCCAGCCGCTTGATGCGGGGGGGGAGGGTCAGGTTGTTACGTTCATACGTTGCGTGAAGATTT
TGCCGGTCAGGTGTTTCTCCTCCGGCACTTCTCTGGCGTAGCTGCCCCAGTCATCGCTGGTCACCATGCC
ACCATGCCGATACAGAACGGAGTCAACAGGGCTAACAGTTCCCGACAGGTCTCATCATTCCGTGGCCC
GCAAGTGTAAGCCAACACACCGCCAGTTTTAGTGTTGTACGCATACCAGGGCCAGTGTTGTTGCGCCTT
GCTGCCAACGAAGCGCCACTGCTCATCAAGTTCACAGATAAGGGCAACATCAGCGTGAGCGACTGGCG
CAGATGTTATCCGCCGGGGCGAGAGCTTTTTAATGTCCGTATGACGGTATTGATGCCGATTTTCAGTGT
TCTGGCGGTATAACAGCCCCCTGCGCCGTTATGTGCCATTTCAACGATCTGCTCCTTAACGCCGGGTTTT
CTGGCTTCATAAGAGTAAGTGAGCTGGAAAACACGTTTACATGAGCGACAACGGAAACGCTCATGCCG
GGAACAACTACGGCCATGTCGATAAACTTCATCGGAATGACAGCGCGGACAATGAACAGTAACGGTAG
TCATGGAAAACCTCAAATACCAGTAGATTACCAGAAACTTCTATCGATTGAAGACATTACCTTTTGCCGC
AATAGGTGGGGTAACATTGGTGAAAACGCATTACGCCGGGTGGCAGTTGGAAAAAAATTATCTTTTTT
CGGCTCTGATCGTGGTGGAGAAAATGCGGCAATAATCTAAATAGCTGCGCTTAATACCGCTACACTTTT
GCCAGCCCATGTTTGCCTCCGGGGAATGGGCTGACGGTTTCCATAAAATGGCGAACTTTTTTCAACAGT
TGCCACATTGAGCTGCACTGATGATTACGCGTTATTGTGTCGTGAAGTGCCTGCCATAGCCGTTCAACA
TGATTCACCCATGGCGAGTAAACCGGCTGATAAATGACCCTGAACTTCGGGTTCTCCTTCAGCCAGCTC
TGTGTTTCCCGGCTTTTGTGGATAATGTAGTTGTCCACGATCAGCGTGATGGTTTTCGCCCGACGGTAT
GTCGCTTTAAGCCGCTTCAGCAGGCTGATGAACAGCGCCGAACTTTTGCTGTTGCCGCCCACACAGCTG
ACTTTACCTGTCCCGCTGTGCAGCGCTCCGGCCAGATAATATTTTTCATTCTGTCCCGGCGTGACCACCC
GTTTTTGCTGTCCGCGCAGTTGCCAGTCCGCACCGATTTTGGGATTAAGATGGATATCCACTTCATCTTC
ATAAAAGACCGGATGCTCTGCGCTGCATTCGTCCAGTGCTTTATGGATTGCTGCCATCTTTTCATCTTTA
TGCGGGTCACGGATACGCAGAGTTGGCGCAGCCCTTCGCCACACAATCCCCGCAGACGGCAACCAGCG
GCGAACGGTTCCGGCATTTAACTGGCAACCGGTTATCTCATTGATTTTTATTGCCAGCAGTTCTGTACTC
CAGCGTGAACGCTGGTAGCCAAAGTCGCCGGGAGAATGTTTTACCAGCTCACGTAACAGTGTGCAGAT
ATGCTCAAACGGCCAGCGACGGGCACGCCCGGCAGGTAATGATTTCAGTCCCTCAACACCCGACTGCG
TGAACCAGTTAATCCAGCGTCCAACAGAGGAACGGGCGCAGCAGAGCGTTCTGGCAACGTCGCTGACA
CGGTCGCCCCGGTGCAGCATCAGCATGGCAGTCAGTCTGCGGGCATAATTTTTATCGTGTGTTTTATGG
ATGGCTTTCTGCATCAGGCGTCGTTCGTCACGGGAAATTGGTGCTATGATCGGCATTGCTCAGTCCGGT
TGGTGATTTGTTTTGATTTGGCGATTGATCAGATCGCACAATCCGGGCTAAGTTCCCTCAAAGTGATCTA
CTATTCCGCGCAGCTATTTAGGYWRWKTCCCWWMATTACGTAAGCGTTGATAAAAGAGTCGGATGC
AGCCCAGTTTCACCATCGCCAGGTAATTTCTCGCCGTTTTGTCGTAACGCGTGGCGATGCGGCGATATT
CTTTCAGCCGCCCAAAGCACCGTTCAACGACATTACGATTGCAGTACGCATCACGATCAAGCTGTGCAC
GTCCATCCGATGCCATTTTCTCATTTGACTTTCGGGGGATTACTGCCTTTATACCGTTATTTTTCAGCTCG
TTGCGCAATGCGCGCCCTGAATACGCTTTATCAGCCAGTACCGCATGTCCACGGCGTTTCATGCTGCCG
TTCTGACGCTGAACGCCAATCCCGTCCAGAAGTCGTTGCGCGAACTGGCTTTCATGAGCTTGTCCGGGG
CTTAGCACGATATTTAACGGGAGGCCACTTCCGTCTGTTGCCAGATGGATTTTGGTGCCAAAACCGCCG
CGAGAGCGACCCGGCCCATTATCTCCGTCGATATCGGGATGTTTTTTTGAGCACCGGCGGCACATTTCA
GCGCCCGGATATTACTGCCATCCAGCGCGGTGGCAGACCAGTCAATAAAGCCGTTTGCATCAAGTAGC
GAAAGCAACCTGTTGAAAATAATATTAATCACGCCTGACTTTGACCATCGGTTAAAGCGGTTATAAACA
GTTTTCCATGACCCATATCGTTCGGGTAAATCGCGCCATGGTGCACCGGAACATAACACCCAGAACATG
CCATTGATGATTTTACGGTGCTCAGCCCATGGGCGTCCGGCCCGTGGTGTGGCGGGTTCAGGAAGTAA
CAGGGGCTTGATGATTCGAGCATTTTCTGCCCGCAGTGCCGATAATTCATCGCTGTCAGATTTGTGAAC
CATGCCCTGCTGTAAATCTCCGCATAGATTATGTCTGTTTGATCTCAGTCATCCGGTGTCTGGCGAGAAG
ACGCACCACGGTGGAAGGGTGAACCCGAAACAGACGGGCGGCATCAGCACCTGTTTTCTGCCCCGATG
TGACGAGCGAGACAATCTCTTTTTGTTGCTGGGGTGTTAACTTTGGTCGCCATCCACCAACCCGTCCTG
GCGTGCCGCTGCAAGTCCACTCTTTGTCCGTTCACGAAGCATGGCGCGTTCAAATTCAGCAAAGGAACC
AACGATTTGCACCAAATACTTTTTACGAGGTTCTTTTCGCCAGTCCTGTAATTCCATACTCAGGTGTCCAT
CTATGTCAGATGGGCGTTTAAGTTACCAATTATCGCTTACTTACGGGGTGAGTATCGTAAAAAAGCGGC
TCTGCAAGTCCTCCTAATTGGTGACATTACCGACCATTTTTCTTCCTTATGTTTTGAGGGTATGTCTTTAA
ATATTTAAAGAAAACTTTAGAAAAATACGATGGTGAACTGTAACCTAACTCAGATGATATGACAGAAAT
GGGAGTATTTGTCCTTCGTAATTTCCTGATAGCTTCTGTAAGCTTTATTTTATGTACAAATGTACTTATAG
AACCGACCTCTTTGTACATTCTTCTTTTTAAAGAAGATTCACTCATTAAAACCGCACTGGCCAACTCGGA
ATTACTCCAGTTACGGGAGTAATTTGCCATCACAATTCTGGCGATTTTTTCAGACACAGAAACAAATGAT
GGTGCATGAAGAATAAGCCCAACATTTTTGTTTTTTTTCAGAATACATGAAAGCAAATAAGAACTCCGA
ACTTTATCGTAAATGGCATCTGTCTCCGTCTTTTTATTATCAGTGCACTGAGGTAGCCTGAGTTTAACGG
ACACTCCTTCCTGAAATAGAATGGCATCAGAAGGAGCTAATAATGAGCAGAAAAACCCAACGTTACTCT
AAAGAGTTCAAAGCCGAAGCTGTCAGAACGGTTCCTGAAAATCAACTTTCGATCAGTGAAGGCGCTTC
CCGATTATCTCTTCCTGAAGGCACTTTAGGACAATGGGTTACCGCCGCCAGAAAAGGGCTCGGTACTCC
TGGTTCCCGCACGGTGGCTGAACTGGAATCTGAAATTCTGCAACTGCGTAAGGCGTTAAATGAAGCTC
GCCTTGAGCGAGATATATTAAAAAAAGCAACAGCGTATTTTGCACAGGAGTCGCTGAAAAATACGCGT
TAATCGAACAATGGCGACAACAATTTCCCATTGAAGCGATGTGTCAGGTATTTGGTGTATCCAGGAGCG
GTTATTACAACTGGGTACAGCATGAACCCTCAGACAGAAAACAAAGTGATGAGCGGCTAAAACTGGAG
ATTAAGGTGGCACATATCCGCACTCGCGAAACATATGGAACCCGGCGGCTCCAGACGGAGCTGGCAGA
GAATGGCATCATCGTTGGTCGTGACCGACTGGCACGTCTTCGTAAGGAGCTAAGGCTACGCTGTAAGC
AGAAACGCAAGTTCAGAGCGACTACGAACTCGAACCACAATCTGCCAGTTGCGCCAAATCTGCTGAAC
CAGACGTTCGCTCCTACAGCACCAAATCAGGTCTGGGTGGCGGACCTGACGTATGTTGCCACACAGGA
GGGATGGTTGTACCTCGCTGGCATCAAAGATGTTTATACGTGCGAAATTGTCGGCTACGCCATGGGAG
AGCGCATGACAAAAGAGCTGACAGGTAAAGCCCTGTTTATGGCGCTCAGGAGCCAGCGCCCACCTGCC
GGGCTAATCCACCACTCTGATCGAGGTTCACAGTACTGCGCATACGATTACCGGGTCATACAGGAGCA
GTCTGGTCTGAAAACATCAATGTCGCGTAAAGRWMMMWWRWKMWYMRMMMWRMMRRCRTM
MMRCTTCAGTGATCCAGGTCCGGTATTCCATACGATAGTGTCCACTTCAAAATGATGGACATTATTTTT
GTGGAACCGGAGGTAACAGACCAGACGGTTTAAATGAGCCGGTTACCTCAAATTCAGTGTATCACCAC
GAGATAATATTCAGCTTTTTATATTCTTCATAATTTCCAGAAAATCACTTCCATTTTTTTTGTCCAAGTGTT
CTGCTATTTCTTCAGGTGTAATATTTTCATTTTTTATTATTTCCATATAAGTTTTTATTGCCTCAAAATTCCT
ATTTTGAAAAGCTATAATTAATCCAGATACCCCATTTGGGCCTTCTGCTGCCAGTAATTTTGATGCCTGA
TGTGGAGTAAGGTCTGCAGTGTGAAGTATTTTCCCATAAGCAATAATTGTTTCTACACATCCATTATGCA
AGGCTACAAACAATCCAGATAAACCATTAGAATCTTTCGCTTCTAACAGCTCTGCTTTTTTATCCAAGTTA
AGTTTTGCCTCATTCAAGATATCACCATAAGCTAAGATTGCATCAGCCTGTCCATTATTCAATGCTAACA
GAAGTCCGGGAACATTGTCATATCTCCTTGAGGCCAGCAAATTTACAATATCCTCTGAGTTGAGGAGAG
GGGGGCTAAGAATGAGCTCACCGTATGCGCGTATAGCATCTGCATGTCCATTTTGAAGAGCAAACATT
AGTCCGGGTATCTGTCTGTGTACTTCATCATAATAATAAGCGGTCAGCAAATTTTTCAGCTTATTGTATT
CGATATTTATTGCTCTCTTCTTGAGAAGCCTTCCGTATGTTTTGATAGTTTCTGCATGCCCATTTTGAAGT
GCTTGGGATAAACCCGATATTCGTCCTCCCATTCCTTCCGCAGTTAGTAGTTCTACCGTTTTATCAAGGTT
AAGTCCAGAAGTTTCCAGGAGTTCTCCGTATGCTAAGATTGTATCTGAATGCCCATTTTGTAAAGCTAAA
AGCAATCCTGACAAACCATCGTCATTTTTGGCTGCCAAAAGTAATTCTAACGTATTGTCTGAAAGGTCA
GATAGTTGTACGACTCTTGTTACTTTTTGGATTAGCTCAGTCAATCCCATACGCATCGCGTGATAAATAA
CTTTGGGGTGAAGTAAATTATTAGGCCATCTGATTATGGAGGACATGCTTGTTGGAGTATGTCTGTCCA
CAAATATAGACATACCGTCGGATATAAGACCATAACATTTCTGATGTTTTTCATCAAGAAAATTATCTAC
AGTCAGTTTTTTTATTTTAGCTAAGTTAAATTTGTTAAACTCTGCTCGTAAATGAGTTGCTGTATGGTTGG
GATCATAAAGCTGAATGACAAAATCCCCCCCATTTTTAGTTTCTTTAATCTTCAAGCCTAATGCCATATTG
TGATTTGATGTACTTATTTCTAGTGCTCTGCTTTTTTCGGAATGAAAAATCATTTCATTGAAAATATAAAA
AATAATATCTCCCCATGAACCGTTTTCAACAAAATAAATAATAGCAGGCACATTAATAGCCTTTTCTTCTT
CACTCACTGAAACATAATTTTTAATTTCATCCGGAAAAGCAAAGTGATGATAATCCACGTGACCACTACT
TTTGTTAAACTGTGCTATCCAGTAGGAAGCTAAATGTCGACAGACAATAAGTCTTCCAGAGTGATCGGT
AACTTGGCAATTTAGATTTAATAAAAAACTCCTCTTGTCAGAAAAATATGGCAATTGTTTTGAGTGCAAT
AGAGACTGGATTGTCTCAACAGAGACGTTGTCTTTATTTATCATATTTTGCAAACATATTTTCCTTGATG
GAATTAAATTTACTGATGGCATATGTATATATTCCTTTCATTGATGGGTTGTGACGCAGACTTAGTGTTG
TTGTACTCTTTGCCCTTTGGATGATTGATGGCAAAGATTGATGTGGTTTGTCCCCGTTACTCTGAAACTC
AGGGGGGTATCCGGAACGGCCATTCCAGTTCAGGGGGGCTGCTCTATCGCTGTAAGCAGTGCCTGAAG
ACCTTCCAGCTCGACTACCGCTACAACGGGGCGAAACCCATCAAACTTTCCAACTATTATCACATTGAGC
CATACTGACAATAAACAATATATCGATATTTTGTGCAAATACTGCCTTTTGCTAAACGATATTCAATTTTG
ATTAAATATATTTATTGTCAGATGTTGCACCATATTTCAATAATAGTITTATCATTTCTGCATTCTCATATT
TTATTGCGTTATCCAACATACAATCCCCACTATTAGTTTTTTTGAACTTTGTGTTTACATCAACCAATCCCT
TATTGATAAAATACTCAAGTACTTTACAGCTTGAATTATGAGCGCTAAGTAGATACTCAATATCATACAA
CTCATGCTTTATGGAAATAACATCTTGTCTAGTTATCTCAAAATTAGATAATATATATAAGGCGACATCC
TCTTTTTGATTAGCAATCGAAAGAAAAAGAGCCTGGAGAGCAATTTTTTTGTTAGTGACCTGCTTGTTAA
TTTCTTCGTAATTAGATGCTGTAACAGCCTCCTCTAAGGATATTTCTCTAATCTTAACTTTTTTGAAGTTTT
CTGTACTTACCATCCTAGGTATATGGTACTCTGGCTGAAAAACAAAGTTTATGATTCTATCCAGATCCAC
AGGGGCAAGATTTTTTCCATAGCAAAACTCCTTGAAGCTTTGGTCTTCACTTTTTCTAATAAAGTCAATC
AGGTATAATCCGAGCCCTAACTTCATATCTTTAGTATTGAATATTGGTACATCTATAATTCCTTTACTTCC
ATGTACGTATCGACTAACTTCTTTATTAACCTCTGAAAATTTATCTAAAAATGATTGGTGCTCATGGTAA
AAAACAGGTGGAATAGCATTATCAAAGTGATCGGTTAATGTCATATATCCATATGGAGAGTCATGATCA
ATGATATACGCATTTGCACCAAAATCAACAGTGTGATGTTTTGTATTCAGGGGGTGTTTTTTCTGATGGC
TAGAGATTTCAACGCCAAAAAACACAAAGTCATGATTAGCTATTTTTTTTATATCATCAGCGTATGTGTT
TCGTTTTGTGCATATTCCTTCAGACTGAATTTTATTGAATGACTTAATGTTTAAAATTTTATCAGACAAAT
CAGAATTACTTTGATGTCTGAAGATTAAATCAGAATTAATAATTAGCGAAAGCAAATTGAGTTCCTCTG
CAGAAAGAGGATGAAGTTTTTCCCTTATTTCAGATATATAATGCTTCGATAATCTGTCAAGGTGCTCAAT
AGCCTTAAATGAGGCTTTAGCGTCTGATGGTGCATCAATTGCTATATTTAAAGATTTAAAGTTAGAGCCT
TGAGAGAACATTCTACTATAACTCTGTGCTGCTATAGTTTTCCTAAAGAAATCTTTAATAATATTTGCACT
ATCTTTTAAATTTTTTTCATCTGGCATAGTTTGTTGTGCAGCGTTTCGCACTGATGTCACTTTTGGAAACA
ATGTATGTAATCTGTTATCCATAGAATCTATATTGCATTGGGTATTTGCTGAGTTCAGTGTTTCTGATATA
TTCATAAAATGAACCTTATATGAAAACAGTTTTAATTAATATCTTTCCCTACAAGATATAGCAGGGTATG
AATTACAATTTTTGAGTATATATACAATATACTGCATGTAATTAGATAAGGGTGTTATATACTTTTGATG
TTATGTGATGCCAGATATTTCAAAATATCTAAAATCCGATGCTTAAGAACTCAAGTTAATCTTTTTTGTG
AAATTATTTTCATAATTTAATAACTATATCAATTTTATTGAACAGCAATGAATTTTACTATAAAAAAACGA
TACTTTCTCTGATTCCTCTTATGGAGTGCTTCCAGTTAGTGGAATGTGTATTCTGCAATGAAATCCTCGAT
GATTTTTTCATGAAGCTCAACTAAGTGTGAAAAACTGATGTTTTTTTAATAGAGGTTAGTTTGGCCATAT
ATAATTATTGTACTTTTGCAGTTTTTTTGTTACTTCCCTTATGAGTTCAAAATCGCCTTAATGGAGCAGTC
ACTGCAACCCAGAGCCTGTGTGGCGTAGATCGCCCGTGAAAATGGAATCTGCCGTTCGGTTTTTGCGCG
AAGTTTTTCTGAGCTTTTACCGAACTACATACGCTGCAGTTTCGCAACCAGCGCCTTCAGTCGGTTGATT
TCGGAAGCATAAGCCGCCATCCGCTGTGAGAGCAGGCGGTTGTATTCAGCCATCTGGTGGATAGTGTC
CTGTTGCGTCTGCAACAGTGCCCGCAGGCGGGCGTTCACGTATAGGAAATTGAAAAACTAAGCGATAC
CCCTGTCCGCGCCTTATGCCACGTGAGCCTTTCTGTTTTTCCCCACCACTCCGGTGTTTTCTCTCTCTGCAT
GGTCGATTCTTATACCGCAACAGGCTGTTTTTTATACTTGCACCGATTCCCGGGCACACTTAGCCTGTGT
CAGGCCGGCATATACCTCAGCAGACTGATATCCCGCGCGTTTTTCTTCAGCCCCTCCACATTCAGCCCCG
CAATGGCCCGGCGGTATACCATCCGGCCGCCACACAGCACGCATTCGAACGGGTCACGACTCAGGAAC
TGTTTCACCATTTGTACATAGCACACTTTCGCCACTGGTTCCGGTTTATCCATCCCCAGAGCACGGTACA
CCTGCGGCAGCTTCTCTCCACACACCCGGTTGGCAAGGAACCCGAAGTACCTCACCATCTTAAAAAACT
TCTCCGGGATGTGCTGTTTCAGCCTCGCGACCAGCTCACGCTGTGTCAGCGTTTCCGTCGCCGTTTCTCC
CGTTTTGTGGTCCAGGTAACGGAAGCTCAGGCTCGCCCCTCCGTTGTAATGTGCCAGCCGGGAGGCCG
CTATCGGCGGCTTCTTCAGATAACGACCCAGGTAGCGCGCCGTATTCCTCCCGCCGGCCGTTTTTTTCGA
CATGTACACATGCCAGTATTTTCCTCCGGCTTTCAGCACCAGGCTTCTCCACTGTGATTCCGTCGTGATA
TGTGACAACGACTCCGGCATCGCCAGCCCCTCTGACCACGCTTTCAGAAGCCGTTGCCGCATATTCCAC
ATCCACCGCGAACGCATCGCGTCTTTCAGGAAGCTCAGCTTTTTCCACTGACCATGCTTATTCAGACATC
CACAGGTTACAGACACATGTACATGCGGATGCCAGTTGAGACGACGGCCATACGTGTGGATGGCGCA
GAAGATACCGGGTTCCTGCCCCCGTTTCCGGGCGGCATACAGCAGATTCTCCACCGCCAGACGGCACA
CGTCATTCAGCAGCCACCGGTTGCTTTCGAACACCGGCCACAGCGTGTCCGGCAGGGTGAAGACCAGA
TGTACCCAGTCGCAGTCAGGAAGACGATTCAGCTGTGTCGCTATCCACAGGTCTGTGGCCTTTTTTCCG
CAGGACGGGCAGGCACGGCTGCCGCATGAGTTAGTGACGTATCTGACGTGGGGGCACTCAGGTTTATC
GCAGTTGTATTCCTTCACTCCCAGTATCCGCGTGCCGCAGGCCAGCATTTTTGTGACAGCCTCAACCTCG
ATATCGCGCAGACCGCCCGCATCCAGGAAGGACGTCCAGCACTGGTTGGCTGTGAACAGGCGTTTCAG
CGGTCGGGGCTGAAAATCACCACCACTTCGGGTCATCCGCCTTCATCTCCGCTTCTGTTTCGTATAAATC
AAAACGACGGCAGGTATGGCAGAACGTGACGTATTCGTACGGTGAATCCGGGTACATTTTGTGGAAG
GTTTCCCAGAAACAGACCGTGCAGGAGGGATGACCGGTGATAAAGTCTGCAAATACGGACTGATAAG
GGTGATTATTGGCTCTGGCGACGGCTTTCAGAACCTCTTTCACCATTCTGGTGTGGACTTTCTGGTGCTC
CAGGTTGTGTGACATGGGAACTCATTCTGGATGGTTACTCTGAAAGCTCATATTCTGCCACACCCCGATT
TGCAGCCGCCAGGCTGCCGTGGTTCAGGTAAACGCCTGTTCGATAAAGCATTACCCAACGACGAAAAC
AAACTCAGGTCGCTAATATCTGACCTGAAACAACATGGTCAGATACTGCTGGTTGTTGATCAGCCAGCT
ACCATCGGTGCGTTACCTGTCGCCGTTGCCCGCTCAGAAGGAGTCCTTGTCGGATACCTCCCTGGACTG
GCCATGCGCCGCATAGCCGACTTACACGCCGGTGAAGCTAAAACTGATGCTCGTGACGCTGCCATCATT
GCCGAAGCTGCCCGTACCCTGCCTCACGCGCTACGCACGCTGAAACTGGCTGACGAGCAAATCGCCGA
ACTCTCCATGCTCTGCGGCTTCGATGATGATCTTGCCGCACAGACAACGCAGGCCAGCAACCGTATCCG
CGGCCTTCTGACCCAGATACATCCGGCACTGGAGCGCGTTCTCGGTCCGAGACTTGAGCACCCGGCGG
TACTCGATCTTCTCCAGCGATATCCCTCACCAGAAAAACTCGCTTCGCTGGGCTTCGCTGGGTGAGAAG
AAGCTGGCAGCCCAGCTCTGCAAACTTGCGCCTCGTCTGGGTAAACGCCTTGCAGCAGACATAGCTCA
GGCACTGGCCGAACAAACCGTCGTCGTTCCCGGCACGAATGCCGCTGCCGTAGTACTGCCACGTCTGG
CACTCCAGCTCATCACGCTGCGTAAGCAAAGAGACGAGGTGGCGCTTGAGGTAGAACAGCGAGTTCTT
GCTCACCCTCTTTACCCGGTCCTGACCAGTATGCCCGGAGTCGGTGTCAGGACCGCAGCCAGACTCCTC
ACCGAGGTCGCCTGCCGCGCCTTCGCCTCTGCCGCACATCTCGCTGCTTATGCTGGCCTTGCKCCGGTAA
CTCGGCGATCCGGCTCGTCAATACGCGGTGAGCATCCCTCGCGACGGGGTAATAAAGCTCTCAAACGG
GCGTTGTTCCTGTCGGCCTTCGCCGCGCTCAGGGATCCGCTCTCCAGGGCTTACTACACCCGCAAAATG
AGTCAGGGAAAACGACACAATCAGGCGCTTATCGCCCTGGCGAGACGACGCTGCGACGTTCTGTTCGC
CATGATGCGCGACGGGACTTTTTATACCCCGCAGGGGTCATAACATGCTTGACAACTTAATAGGGGCCC
CCCCCTAAGCCGTCTATGGGAAGGAGTCTGCTGTTATTCCAGAGAATGGTAATTCAGTCATATATTTCTC
CATCTATGACTTGTCAATACCATCCTGATATTTGTGGGTCAGCAATATATTCTACTAAAAGGTATTTAAG
ATCATTGCGTCCCTGCACTCGAATAATGGTGAAGAAAGCATGATGAATATATCACGATAACGTTCAGTA
TGTATCGAGGATAGATGGAGTTACTCCTCTCTGAAATAGAATTACTGCTCTACTTTTTCTGTTTCCAGAA
ATTACTAGTAACAAACCCTGATATATTCTTTTGAAAATGGAGCTTAAACGAGTCTGAAATTAACTTCAGA
TATTAGAATGCCGTTGGCTTTGTTTTTCAACAATTCCTACTTCATTTATCTTTGTCAACCATTGCATTTCTG
TAGCAGTGAATTTCAACGTTTTCGTCCGGTTGGCAGCAGGTATTTGTAATTCGTATATAGCTGCTTTTTT
ATTGAAAACAGTATATCCATCTGTAGTAACAGTAGAAGATAATAATACAGGATTTGATACTTCAAGGAT
GATATTTTCTTGTTTTTGTGGAAGGCAAGGGAATATAGTTTGTGTAAGCATATTGCTTCCTTATGTTATA
TTTTAGTAAAGAATTTATTTTTTACATTATAGTCGGCTAAATGAAGTTTACTGTAAAAAAACGATACTTTT
TTCGTAAGCGATGAGATGTGGAGCCAGAAAAATGGCTGCGTTACGTCATTGAACTGTCCTTGACCCAC
GATATTCAGACAGTCGTTGTATCTTAAGTTGACATTACCCGCTGACGTCGATATTCCCTTGGTGAGAGAY
MMMYMARMRMMMWRWRMRRMWRRWWWWMMWWRWMMWKYWMRMWMRMMMMW
RMMMRRWWMWRMMMWRMMRWWMWYRRWWWMRRMMKRMMMRMRMWRYMRWMW
YSMWWYMWMRWWWWMMMRMMMMKWWMWRMYMWYMMMWWMMWMWRMKRRM
WRMWRMYWRMMRWYRWKMMRRRMWMYMRMWWMMMMWMWMWRRMRMMMWRMM
RWRWWWMMWRAGCCGTATAAGCCGAACCGTTATCGGCCACTGGATTGGCCGTTCCGGTCCTTTAT
CGCCATATCGCTGACATGACATCCTGCACYGTYGCTTTRTCRTAAYCTCCCGTGCYTGCKGMMMMRW
MWMWGRMTTCACGATCGCAATATGGAACGTAACGCTGTCATCGGGAGAATCGTCAATGTGGCATTC
GGTTMASRYWRMWMWRMMWWWMMRWRWYMMMMMRRMMWMWMKMMWWWWRMY
RMWWWWMWWWWWMRRWWWMMWWMMMWRMRRRMRMMRWMKMMRWMMMKMY
RMMRRWRRMWMMMRMWRMWMWWMRMRRRWWRKMWMSMGMMWTACAGAGGTCCGTC
ATATGAGCCTGCCACTTTCTTACGTCGCGAATCAGCTTTGTTCCGCCGTCGCCAAGCGCTAACGTGSGTT
ACTCTTGTACCGGGCTACGTCGCCTTCAGGGAACTGGTTATTCTCATTTTTTATTTGTGACATTGTGGTT
GTTGTGCGACAGACATTGCTATTCGGCCCATATCCCAGATAAAACCCGCCAGCTCACGTGCAACAGCAA
CAATTGTAATATTGACATTCTTTCCTTTGGCCTGAAGTTTTCGATACCTCTTACAAAGCCTGAGTTGAGCA
TCCCATGCTCTGTCAATGACGGGGGGGGTAAATTTTCCTGCCTTTTTTGTATTGCCGGACTGATGCGTG
CCGGATGACGGTAACTCCACGCTGCCTCGACCAATAGCTTTCTGGCATAGCTGTTTCCGGCTTTGGTAA
TGCTGCCCTGATGTCTGCTTCCACCCGATGAATATTCTGATGGCGTGATACCAAACCATGACATCAGTTG
TCGTGGATGTTCAAAACGGGTCAGATCACCCAGTTCAGAAATTAATCCAACCGCAGTAATAAACTGAAT
CCCTCGCATGGCTTGCAGCGCCTCTACTACCGGATACAGCCTCCATTCGGTAACGGCCTCCTTCAGGGC
GGATTCCAGCCGTTCACATTGTGCCTGCCGATCTTCTATTGTACGACGGTGTTCGTCAAAAGCTAATTGT
CGCCAGGGACTTTCGAATGAGTATTTGCTGAGCCAGCGTCGGTGTGCAGGGCCCCAATCTGCTCGCCC
GACATAATGGACCCCATGAACCAGAAGAAACGATTTCAGACGTTGCCTTGCATGCCGTAAATCATCGCG
GGCAGATGCCCATGCCCGGGCCAAATCCCGGAATGCCTCATCTTCAATGCCGGGTACGTAGACAGCAG
AAAGATCTCCTGCACGCAGTGAACGCACAAGTCTGATGGCATCACGGCGATCGGTTTTAACCCGCTCCC
CCGGTTTTTTCGGGATAAGTGAGGGGGCGCAGACCATGCAGTCAAAACCGGATTTCACCAGCCGACGA
TAAAGTCCATAACCACAGGGCCCCGCTTCATAAACGATACTGACCTGCGATGACTTTGACCTGAGACGT
TTACAAAGATTCTGAATATCAGTAGGTGATGTGCCAATTTTACCCATCAGTTCAACAGGTTCTGAATTGA
TAGCATAAGCGACAGTAATCGATTCTTTGTGAACATCTAACCCGACGTACAATGTGTTATTGTTGTTCAT
GCTGGCCTCCGTGGTGGAAATACCGGGGTGTAGCAACAGCTCACCCATGCGGCTCTGACAACGATGTT
AACCCGCGTTAAATATCCCACGTTGGCCAGCATCTGTCTCACGGAAAGTCATACTGCCTTACCAATATCA
ACACCAACCGGATTTGTGGAGCCTCCAGTCTGAAGGGGCTTTATGCGTTCCGTATAGGATATTATGATG
CTGGAGTTTTGCGAAGCTCCACTCCTTTCCGAACATTTACACAGATATTGCCTAAGAAAAGTGAAACCAT
AATATGGTTATAGTCCCTTTCAGTGCAAATACTTAGCTTGTTGCACAGAGAAATAGAAGCTGCATAAGC
TCTTTCTTCAAAAAATGTAAATAAAGTTAAATATAGGAAAAATTACTTAATCTATCTTAATAACGGAAAG
ATTTTGTATACAATCACTGTTACAGAAATTTCTTAGTTACTCTGTAAACACTAAATATAGTTTGGTATATT
CTGTTGAATTTATGATGGATATGGGACATAAAAACAAAATAGATATAAAGGTTCGCTTGCATAACTATA
TTATTTTATATGCAAAAAGGTGTTCAATGACGGTTAGCTCAGGCAATGAAACTTTGACTATCGATGAAG
GGCAAATTGCTTTTATAGAGCGAAATATACAAATAAACGTCTCCATAAAAAAATCTGATAGCATTAATC
CATTTGAGATTATAAGCCTTGACAGAAATTTATTATTAAGCATTATTAGAATAATGGAACCAATTTATTC
ATTTCAACACTCCTATTCTGAGGAGAAAAGGGGGTTAAACAAAAAAATATTCCTCCTCTCTGAGGAGGA
GGTTTCTATCGATTTGTTCAAATCTATAAAAGAGATGCCTTTCGGCAAAAGAAAGATCTATAGTTTAGCT
TGCCTTTTATCAGCTGTTTCTGATGAGGAAGCTTTATATACTTCGATATCGATAGCTTCTTCTCTTAGTTT
TTCTGATCAGATAAGGAAGATTGTTGAAAAAAACATCGAGAAGAGATGGCGTCTTTCTGATATTTCAAA
TAACTTGAATTTATCAGAAATAGCTGTTAGAAAACGATTGGAGAGTGAAAAATTAACATTTCAACAAAT
CCTTCTTGATATTCGCATGCATCATGCAGCAAAGCTTTTATTGAATAGTCAAAGCTATATTAATGATGTA
TCAAGACTTATCGGAATATCAAGCCCATCTTATTTTATAAGGAAATTTAATGAATATTATGGTATAACTC
CAAAGAAATTTTACTTATATCATAAAAAATTTTAAATGCTTCATAGCCCATCGCTATTGCCAGATGGGTT
TTCCCCACACCTGATGGCCCCAGTAATACGATATTTTCATTACGTTCACGTATAGGAAATTGAAAAACTA
AGCGATACCCCTGTCCGCGCCTTATGCCACGTGAGCCTTTCTGTTTTTCCCCACCACTCCGGTGTTTTCTC
TCTCTGCATGGTCGATTCTTATACCGCAACAGGCTGTTTTTTATACTTGCACCGATTCCCGGGCACACTT
AGCCTGTGTCAGGCCGGCATATACCTCAGCAGACTGATATCCCGCGCGTTTTTCTTCAGCCCCTCCACAT
TCAGCCCCGCAATGGCCCGGCGGTATACCATCCGGCCGCCACACAGCACGCATTCGAACGGGTCACGA
CTCAGGAACTGTTTCACCATTTGTGCATAGCACACTTTCGCCACTGGTTCCGGTTTATCCATCCCCAGAG
CACGGTACACCTGCGGCAGCTTCTCTCCACACACCCGGTTGGCAAGGAACCCGAAGTACCTCACCATCT
TAAAAAACTTCTCCGGGATGTGCTGTTTCAGCCTCGCGACCAGCTCACGCTGTGTCAGCGTTTCCGTCG
CCGTTTCTCCCGTTTTGTGGTCCAGGTAACGGAAGCTCAGGCTCGCCCCTCCGTTGTAATGTGCCAGCC
GGGAGGCCGCTATCGGCGGCTTCTTCAGATAACGACCCAGGTAGCGCGCCGTATTCCTCCCGCCGGCC
GTTTTTTTCGACATGTACACATGCCAGTATTTTCCTCCGGCTTTCAGCACCAGGCTTCTCCACTGTGATTC
CGTCGTGATATGTGACAACGACTCCGGCATCGCCAGCCCCTCTGACCACGCTTTCAGAAGCCGTTGCCG
CATATTCCACATCCACCGCGAACGCATCGCGTCTTTCAGGAAGCTCAGCTTTTTCCACTGACCATGCTTA
TTCAGACATCCACAGGTTACAGACACATGTACATGCGGATGCCAGTTGAGACGACGGCCATACGTGTG
GATGGCGCAGAAGATACCGGGTTCCTGCCCCCGTTTCCGGGCGGCATACAGCAGATTCTCCACCGCCA
GACGGCACACGTCATTCAGCAGCCACCGGTTGCTTTCGAACACCGGCCACAGCGTGTCCGGCAGGGTG
AAGACCAGATGTACCCAGTCGCAGTCAGGAAGACGATTCAGCTGTGTCGCTATCCACAGGTCTGTGGC
CTTTTTTCCGCAGGACGGGCAGGCACGGCTGCCGCATGAGTTAGTGACGTATCTGACGTGGGGGCACT
CAGGTTTATCGCAGTTGTATTCCTTCACTCCCAGTATCCGCGTGCCGCAGGCCAGCATTTTTGTGACAGC
CTCAACCTCGATATCGCGCAGACCGCCCGCATCCAGGAAGGACGTCCAGCACTGGTTGGCCGTGAACA
GACGTTTCAGCGGGGGGGGAGTAAAAGCGGACAACATGAGGACGGATTAACCCCCGGTATCCTGTAG
CCGGGTAAGGGCTGCTGTGTCGCCACGGCGCTCCAGGATGGAGGCCGCGACCATCGCAGGTGGTGGG
TCCATGGGTTTGAGCATCTTCATCCAGTCCCATTCACGTTGTGAGAGCTTCTCAGCACCAACAGAGAGC
AGAACGGTATCTATACTTTTGCGGGTCAACATAAGAGGATGAGTCCTTAATAAAACAGGTCCTCAGAGC
ATACATATTCTGCGGAGCGTGCGGCAACAACAGGACGAACAGGGGCGGTTTTCAATTTGCAGCCGTCA
GGCTGCCGTGGTTCAGCGCCTGATGGGAATCCTCTCACTTAAGGCAGCGATTAAGGTCAAGCGATACC
GCTCTTACAGAGGAGAGGTAGGGCAAACCGCCCCCAATGTTCTCCAAAGAGATTTCAAGGCTACGCGG
CCAAACGAGAAGTGGGTTACCGATGTTACTGAATTTGCAGTCAATGGGCGCAAGCTGTATTTGTCTCCA
GTAATAGATCTCTTCAACAACGAAGTTATTTCTTACAGCCTTTCGGAAAGACCAGTGATGAACATGGTC
GAGAATATGCTCGATCAGGCATTCAAAAAGCTTAATGAACCGCCCCGGGAATCCTGGAGACTAAACTT
CCTGAGAAAGAGGTAAACAGGATGACTAAAAATACTCGTTTTTCCCCCGAAGTCCGTCAACGGGCAGT
CCGTATGGTTCTGGAAAGTCAGGGCGAATATGACTCACAATGGGCGACAATTTGTTCCATTGCTCCAAA
GATTGGCTGTACGCCGGAGACTCTGCGTGTCCGGGTTCGCCAGCATGAGCGGGATACCGGGGGCGGT
GATGGAGGGCTCACCACCGCTGAACGTCAGCGTCTGAAAGAGCTGGAGCGTGAAAATCGTGAACTGC
GCCGCAGTAACGATATCCTTCGCCAGGCTTCCGCTTATTTTGCGAAGGCGGAGTTTGACCGCCTCTGGA
AAAAATGATGCCACTGCTGGATAAGCTGCGTGAGCAGTACGGGGTCGGACCGCTATGCAGCGAACTG
CATATTGCCCCGTCAACGTATTACCACTGTCAGCAACAGCGACATCATCCGGATAAACGCAGTGCCCGT
GCGCAGCGCGATGACTGGCTGAAGAAAGAGATACAGCGCGTATACGATGAAAATCACAAGGTATACG
GTGTGCGTAAAGTCTGGCGTCAGTTGTTACGGGAAGGTATCAGAGTGGCCAGATGCACTGTGGCACGT
CTCATGGCGGTTATGGGACTTGCCGGTGTTCTCCGGGGTAAAAAGGTCCGTACGACCATCAGCCGGAA
AGCCGTTGCCGCAGGCGACCGCGTAAACCGTCAGTTCGTGGCAGAACGACCTGACCAGCTGTGGGTG
GCTGATTITACTTACGTCAGCACATGGCAGGGCTTCGTCTATGTGGCGTTCATCATTGATGTGTTTGCCG
GATACATCGTGGGGTGGCGGGTCTCATCGTCCATGGAAACGACATTCGTGCTGGATGCACTGGAGCAG
GCGTTATGGGCCCGTCGACCGTCCGGCACGGTCCATCACAGTGATAAAGGTTCTCAGTATGTATCGCTG
GCCTACACACAGCGGCTTAAGGAAGCCGGATTACTGGCATCAACAGGAAGTACAGGCGACTCGTATGA
CAACGCGATGGCGGAGAGCATCAATGGTCTTTACAAAGCGAAGGTAATACACCGTAAGAGCTGGAAA
AACCGTGCAGAAGTGGAACTGGCCACACTCACGTGGGTGGACTGGTATAACAATCGACGATTGCTGGA
AAGGCTGGGCCATACTCCTCCGGCAGAAGCAGAAAAAGCTTATTATGCTTCCATCGGAAACGATGATCT
GGCAGCCTGAGTTCACAGATAAAACACTCTCCAGGAAACCCGGGGCGGTTCAGTTGCTGAACTGCTGG
AACCGCTGTATGACATCCTGCGCCAGTATGTGCAGATGCCCGGTAAAGTCCATGCCGATGATATCCCCG
TTCCGGTCCAGGAGCCGGGCAGCGGTAAAACAAGGACTGCCCGGCTGTGGGTCTACGTTCGTGATGAC
CGTAACGCCGGTTCGGAAATTGCAGCATAAGTTATCCATGCTCTGAGGTGGGTGGAGGTRGGMMMT
MMRRTCATGTCAACGCCACGATGTTTGACCGTTATTTGCCATTTTCATGCCCGCTAAATACTTTCGTTAC
TGATGCTGTCCGCTTTCCACCATTTCATCATCACTGATTGGCGTTGTGTTTTTGGTCAGCACTCCAGCTTT
ACGTTTCTCTTTTAACCGGTAACTCTCTCCTTTGATATTCAGCGTGGTTGAGTGATGTAGCAGCCGATCC
AGGATCGCTGTTGCCAGTACGTGATCTCCGAACATTTCTCCCCAGTCTGCGAACCCTTTGTTTGACGTCA
GTATGATGCTCGCTTTTTCATATCGACGGTTCAGTAGCCGGAAGAACAGACTGGCTTCCTCTCTGTTCAT
CGGCAGATAGCCTATTTCATCCAGGATCAACACCCGGGSMTMAMTMRKWWRMKRYMRMWRMMS
SYMMMSMYRRYWWWYMWRWWWSRMWWWMMWMMRWGWMRCGATCAGTCTGTCCAGTG
GCATAAACAGTACCCGATGTCCCGCATCCACCGCTTTCACGCCAAGAGCTATGGCCAGATGAGTTTTTC
CGACACCAGGAGGGCCCAGCAGGATCACGTTTTCGCTGCGCTCCACGAACGCCAGACCAGCCAGTTCC
CGGACAACCTTACGGTCGATGCCCGGCTGGAAGGTAAAGTCGAACTGCTCCAGCGTTTTGACCCACGG
CAGACGAGCCTGCTTCAGCCTGGACTCCATACCGCGCTGATGCCTGCCGTTCCATTCCTGCTGTAGCGC
CATGCACAGGAACTCCCGGTAGTTCAGCTCTTTTTTAGCTGCCTGTTCCAGCAGGCTTTCAACGTGATAA
CTCAGATGCTCCATTTTCAGGCGACTCAGCAGGACTTCCAGTTCATGCATCACAACAGCTCCTCATAGGC
ACTCAGTGGTCGATGTTCCACCTGACTGACCTGCTGCCAGAGCGGGGCGTGATGCTCCGGCACTGTCT
GCCAGCCAGACGATGCTGAACAGAGGCGATGTGAGGCCACCAGTTTCTCATTACTGTAGATCCGCAAC
TCGTCATCCAGCGATATTCGTATCGATACCGGCTGACCACACAGCGCTTCGGGAACGCTGTAACGATTA
CCACCAACCTCGATATAGCTGTCCCAGGACACATGGCGGATATCGAAGTAACTGGTATCGAAGTCTGTA
TCCGGTAACGGCTGCAGATGTTCCTGCTCCAGCGCGAAGCGCTGTTCCGGCGTTTCTTTGAACTGGCGA
AGCTCCCGTTTGTCAGCCACATCGGCTATCCATTGCTCCAGTTGTTGATTGACATGAGTGAAGCTGTCG
AACCTGCGGTACCGGACGAAGAAGTTCTCCTTGAGGTATTTCACCATCCGCTCAACCTTACCTTTTGTTC
TGGCCCTGCGTGGACGGCATGCCCGTGGCAGGAAGTTATAGTGGTCAGCCAGCAACAGGAATCCGGA
GTTGAACACGACTTTCCCGTTGTTATTCTTCAGCACCGCAGCCTTCTGGTTATCAACCAGCACCGTTTTCA
CACAACCACCGAAGTAGCGGAAGGCGCGAACCAGTGATTCGTAGGTATGCTCAGCATCCTGTTTTGGT
GCGGCGAAGACATGGAAGCTGCGGGAGAACCCCAGCGTATTAACCGCAAAGTTAACTTTGCACCGTTG
CCCGGCAACCTCCACCTCAACTTCGCCCCAGTCATGCTGGAGCTGGTATCCAGGCTGAGTTTCGAAGCG
AACTGTTCTTTTTGACGGACGCATTTTACGTTTGGGCTGGATGTAGTAACGCAGCATGGAACGTCCGCC
CGTATAACCCATTGCCTTAATCTCCGCGAAGATAACCTCGCTATTCCAGACATTCTCTGCCAGGCGCATG
TCGATGTAATCCATAAACGGTTTCAGCTTAACCATTTTGTGGCGGGTCTTTCTGGCTGGCGGTTCAGGG
TATTTGAGGTAGCGTCTGACCGTCCGTTCAGAGCAACCAATCTGAGTCGCAATATCGACGATGTACGCG
CCCTGCTGGCGCATTTGCTTTATCATGTAAAAGTCCTCTCTGCTCAGCATGTTGATGTCCTTTCTGGTGTG
AGAACCTTAAGGAAACAACATGTTGGGTGGAGCGGACAATCCAAATGGCGAATTACCGTCTTATATCA
CTGGCGCTGACAGTCAGGGTTTACAGGAGCACTGACCAAACGACCCGGTGCTGCTTCATTCTAAGGAA
TGAGGTGGTGAGCGACTCGGGAAAAGGCAGAGATCTACTGTCACGTGGGGTTCGGGAAGCTGAACGA
AAGTAAACTGTCCGATGAAGCATCGAAATCTTAGATGATGTCAAAATGGGGCCGTACAGCCGCCCCCT
GATAAGCGCAGAAGACACCTGCTTACTGACTGCGCGGCATCCGGTGTGAAGGCAGCAGGATCCCGGG
CCAGACCATCTCAGGGAACGTGAGAACCTGTTTCCGGCACGTATCGGGAAACAGGGGCGGACCCATTC
GTAGTAGTGATGAAAGATCTGTAATGGGTCTGGAGCGAAGGGATGGGGATCACATTCATTGCAGAAG
ACACAACCCAAAAGGGGATGACGTAACTGATGCAACGAAAATCATTTGAAATACCAAAAGCACTGGTC
TGGGCATCATATCTGGATGTGCGTCGCAACAAGGGAGCCCCCGGATGCGACGGGCAGACTTTGAAAAT
GTTCGACCAGCAACGTGATGGCAATCTATACAAGATCTGGAACCGGCTATGTTCGGGAACATGGTTTCC
GCCACCAGTGCTGGAAAAACGGATCCCGAAGTCGAATGGCAAGGAACGCATTCTGGGGATCCCAACA
GTATCCGATCGAATTGCTCAGGGAGCGATAAAACTTTTCATGGAAGAAAAGCTTGACCCGATTTTCCAT
GCTGATTCATATGGTTACCGACCAGGCAAATCAGCTCATGACGCTCTGAAACAATGTGCCATCCGGTGC
TGGCGTTATAGCTGGATACTGGAAGTGGACATCAGTGCGTTTTTCGACCATGTGAGACATGATCTGGT
GCTCAAAGCTCTGGAACACCACGGGATGCCTAAATGGGTCATCCTGTACTGTCGACGCTGGATGGAAG
CGCCAATGCAGAGTTGTGAAAATGGAGAATTAATAACCCGAACACGAGGCACTCCGCAGGGGGGGGT
CATTAGTCCGCTACTTGCTAACTTGTTCCATCACTATGCATTTGATTTGTGGATGGAAAGAGAATATCGG
GGGTACCGTTTGAGAGGTACGCTGACGATATTGTAGTACATTGTTCACGAATGAGTGATGCAACAAGG
CTGAAGAACAGGTTATCGGAGCGCTTCTCGGAAGTCGGGCTGGTCCTGAATGCAGGGAAAACGAATAT
CGCCTACATTGACACGTTTAAAAGGAGAAACGTCGCAACGAGTTTCACCTTTCTCGGATATGACTTCAA
AGTGCGTACGCTGAAGAATTTCAAAGGCGAACGGTACCGAAAATGCATGCCGGGTGCGTCAAATGCA
GCAATGCGCAAAATAACAGAAACAATCAAGAAGTGGCGTATACATCGCTCAACAGCTGAGAGTTTGCT
GGATTTTGCGAGGCGCTACAATGCGATAGTGAGAGGCTGGATCGAGTACTACGGAAAGTTCTGGTCCA
GAAATTTCAACTATCGACTGTGGAGTGCAATGCAGTCACGTCTGCTCAAGTGGATGCAGTCTAAATACA
GACTTTCGAACCGGAAGGCTCAGCGAAAGCTGACGCTGGTAAGGAAGGAGTATCCGAAGCTATTTGTT
CACTGGTATTTACTACGTGCATCAAATGAGTGATCAAGAGCCGTATGACGCGAGAGTGTCACGTACGG
TTCCGTGAGCAGCCGGGGAGTGAAATTCTCTCCGGCTGACTCGACTCGCGGGTTCCGACAGCGATGAC
GAACATGCGGCGGTGTTGTACTCGCTGATCGGCACATGCCGTCTGAACAATGTGGAACCAGAAAAATG
GCTGCGTTACGTCATTGAGCATATCCAGGACTGGCTGGCAAACCGGGTACGTGATCTGTTGCCCTGGA
AAGTTGATCTGAGCTCTCAGTAAATATCAATACGGTTCTGACGAGCCGCTTACTTTTGGCGTAAATCTGC
TGCGCCTTTACTGAACACAATGCCGTATTCAGCAAGAAGTCCACGAATGTGATTTATCAGTTGTGTCCTT
TCCATTGAAACCAACCGACGCATATTCAAAAAAGTCTGGACGTCCTGTTGCTCCAGAGTTTTTATTGGTA
CGGGTCGTATAGTTCCATGTTGGCAGGCCTCAGCTATTGCCTGTGCGTCGTTATAATCATTTTTCTGGCA
CCGCTGATAAGCTTTAACATGTTGTGCCGGAAGCAATATTACATCATGGTCGAGTTTCCTGATTTCCCGG
GCCCAGTAATGAGATGCCCCGCCAGCCTCCATCGCAACAGTCATTTTAGGCTGTTGCACCAGCCAGTTA
AGCAGTTGATTGCGAGAAAGTTTGGTCTTTCCTGCGGGTTTGCCATGAGCATTGATACTGAAAAGATAA
AAGTTCGTTTTAGCCAGATCAATGCCGGTGATTTTCTGTTCAGACATGGTATTCCGCTTACTTTTGTGGT
TAAAGTTTATCCATCATGGCGCAATTGAAGGCTGCCGTGAAAGAGGGGGAAGACTATCTCATCGGTTA
CTGTTAAACGGCTAAGAGAAACTATCTTTTTCAAGCAAAGAAGATACGGTGAAACAGGCGATTTCACC
GTGAAATACTGTTCAAAAAAAACGCTAATAAAAGCAAAATTTCTTTTAAAACAGCCAATTAAAAACTGG
TGAGTTGCTGGCGAAAAACTTGAATTTTCACAGCGAATTCAGATTATCTTTTAGTACAAGAGCAATCAT
ACGATCTAACTTATCCTGAACTTCCAACGGTAATCTGCCGAATTCATATGAGAACGTACGTCCCTTTACT
CGTTTTCGAGCAAATTTATCTTTACTATCAAATTTCCATAACAAAGTTACAACCGCTTTATCCTTTACTCCT
GAATCAAGCAATGAAGACATTTCCTTGGTTATAAGCCGCATAATACTGTTTTTAACCTCGTCTTCAGAAA
GATTGTCATCAGCAGTAATAAGCTCAATACTCGGCGAGATATTCTTCACAACTTCATCAATAGATATATC
ACCCAGCCTCAGTCGTTCACTTAATTCAGCCAGTTGCCTGTAATCGGCGTATGTCAGCTCAGACTGAACA
GGAAAAAGAGACACAAAATCTTTAGGTACGGACGCTGCTTGGAGGGCACGAGTCACCTTAGCTGCCGA
CAATCCCTCTCTTTCCGCAATTTGCTTCTGGTTCATACCGGCCTCTTTGAGACGTACTAAACGCAACCCAA
TTTCACGAATATTATGTTCAAGTGACGTCTGCAAATCTTTCGCCAGATGCTGAGCTTCAGAAACAGTAA
GCTCATCATCCGTTACAAGTACACGTAATCCAACCTTGCATAGCAAAGCTGCTGCCCGACGACGGGAAC
CATCCAAAATCTCAATAAGATCGCCAGTCCGAATCCCAATACATGGATAGAATTGTTGTAAATGTATAG
TTCTGGTGATGTCTTTTAGTGAGTCCGGCGTCAGGGCTGTCTGTTCGCGGCCATTCACCTCTTCAACCAC
AAAAGTTTGAGTGTCCACTTTGTCATGATCAACCTTGATCTCAGTAAATTTTGCCTTTCGGCCAGTATTG
AGGGTAAATACATGAACTGGAGCCGATATTTCTTCAGTATTATTCAGGATATTTGTATTCAGCGTACGG
CCAATAGTCGGTCGGTGCTTTCTGTTTTCCATCAGTAGTTGGCCCTTATAAATTCAATACGATCAAAAAC
TGCTTTAGCGAAATCTTCTGCTGCCATACGTGCATTTTTCAAAGCTTCACCACTGCCAACATAAGTAACA
GGATTCGCAGATATCACGGTATCAAACGACTCGCCGCTTCTCTCAAAACCATCAAGACGTGGCATTGAA
ACATCAAGCATGTCACCACCGAAAATCTCTTTTGTCAGGCTATGGCAGTATTTATGATCTGACTTATTCG
CCAGTTTCGACATGAAGCCAATATTTGCCTGCAAACGGCATGAACATCCATCCTGTTCAATAATCTGTAC
AAGTTCAGGTAAACGAGCCAAATATTTAAGCGTGGAATGGAAATCAACTTGTGCTGGCGGCACCGGTG
TAAACATGATATCTGCGGCAGCAATGGCATTTTTCAAAAATGCGTCCAGATGCGGGCCTGTATCAATAA
GGATGAAGTCGAAATCATGCTTCAGCTTATCAATAATATTCTCACGCAATATCGCGTGTTTGTTTTGTCC
TAATAGATGCTCCTCGCACAGAGTATCCCAGTTTGATGCAATAAAGGCGTCGTCAATAGAAGCCGGCAT
AACATAAACGCCAGGGATAACTGACGGAACGATAAAATCTTCCAGTAGTTCCTCACGACTCACGTTCTG
CAACATAGCCTGCGGTGCGGTTGTATCAACCAGCCCCACCGCATGCAAGTAGTTCAGAAACATAGTGG
CTGATGACTGGGGATCTAAATCAAGGAGCAGAATACGTAAATCTTCTGACAGCAGGTGAGGATGAGCA
CGCAGGGCGTGAGCTACGGAAACACTACTCACGGTTTTAGACACACCGCCTTTTAGACTACCGATGAAT
ATGGAATATGCCTCACTATAACGATCGCGATATTTAGGAATGCCACGATGTGCATAGATATCGATAATG
TTCTGGATGGTTAATGCATATTTTGTCGCGGTTCCTGCTGGGCGTTTTTCAAACTGATAGCCCTGAGCCT
CCATCTCACCGACTGCACCATCCACGCTTGCGCGCGTTAATTTTGGAAGCTTAGCTACAGCAGACTTTGA
ATAGACTTGATAAAAAACATCAGCCTGAAACTCCTGCTTTTGCTCCTGAATCTGTTTTGTTAGGGCGAGC
AACATTTTATCTGCACGCTGCGCAACCTTACTTAACTGCTCAAAACTAGTCATTCCCCCTCCGTGCTATGT
AGGATTTTAACTTATTACAAAAAAAACTGCATTAGCAGGGTAATGCATGCAAGTACATATTGTACATGC
TTATATTAAAAATGCGCGCATTGCTGATTTATTAATCATCGGAATGAATATTACTGTAAGATAGTATGCC
TTTCCGGGAGAAAGACGCTGATTTCCCCCGAATAATCCAGTTCACCACCACACTCCGGACAACAGCTTT
CTTCAGGCTCCAGATGGTGTATTTCGCGGGAAACATGTTCCGGTAACGGACGGCGATGACGTGACTGT
CGCAACAGGCGGGGGTAGTGCTTCCAATCAGTAGAACATGTGCTTTTCAATGAAGGAGCCGATGACTT
TTTCGTGGATTTCCACGGAACGCGAGAAGCAGATTGTTTTACGTGCCAGCCGCTTGATGCGGGTGCGG
AGGGTCCGGTTGTTACGTTCAATGCGTTGCGTGAAGATTTTGCCGGTCAGGTGTTTCTCCTTCGGCACTT
CTCTGGCGTAGCTGCCCCAGTCATCGCTGGTCACCATGCCACCATGCCGATACAGAACGGAGTCAACAG
GGCTAACAGTTCCCGACAGGTCTCATCATTCCGTGGCCCGAAAGTCGAAAGTGTAAGCCAACACACCG
CCAGTTTTAGTGTTGTACGCATACCAGAGCCAGTGTTGTGTCAACGACGGATGAAAAGTGATCCACTTA
TATCTCCACCAACGGCCCAATATTGATCCACCGTTTTACTCAGGATTAGCTTCTGCTATAACCCCGGCCTT
TCGTTTCTGTCTGAGTCGATAGCTTTCTCCTTTGATTTGAACGAAATAATCCGACGGTGAATCACGCTGA
TATTCGCGGGTTGGATACCGCATTACAGAGGTCCGTCATATGAGCCTGCCACTTTCTTACGTCGCGAAT
CAGCTTTGTTCCGCCGTCGCCAAGCGCTAACGTGGGTTACTCTTGTACCGGGCTACGTCGCCTTCAGGG
AACTGGTTATTCTCATTTTTTATTTGTGACATTGTGGTTGTTGTGCGACAGACATTGCTATTCGGCCCATA
TCCCAGATAAAACCCGCCAGCTCACGTGCAACAGCAACAATTGTAATATTGACATTCTTTCCTTTGGCCT
GAAGTTTTCGATACCTCTTACAAAGCCTGAGTTGAGCATCCCATGCTCTGTCAATGACGGGGGGGGTA
AATTTTCCTGCCTTTTTTGTATTGCCGGACTGATGCGTGCCGGATGACGGTAACTCCACGCTGCCTCGAC
CAATAGCTTTCTGGCATAGCTGTTTCCGGCTTTGGTAATGCTGCCCTGATGTCTGCTTCCACCCGATGAA
TATTCTGATGGCGTGATACCAAACCATGACATCAGTTGTCGTGGATGTTCAAAACGGGTCAGATCACCC
AGTTCAGAAATTAATCCAACCGCAGTAATAAACTGAATCCCTCGCATGGCTTGCAGCGCCTCTACTACC
GGATACAGCCTCCATTCGGTAACGGCCTCCTTCAGGGCGGATTCCAGCCGTTCACATTGTGCCTGCCGA
TCTTCTATTGTACGACGGTGTTCGTCAAAAGCTAATTGTCGCCAGGGACTTTCGAATGAGTATTTGCTGA
GCCAGCGTCGGTGTGCAGGGCCCCAATCTGCTCGCCCGACATAATGGACCCCATGAACCAGAAGAAAC
GATTTCAGACGTTGCCTTGCATGCCGTAAATCATCGCGGGCAGATGCCCATGCCCGGGCCAAATCCCGG
AATGCCTCATCTTCAATGCCGGGTACGTAGACAGCAGAAAGATCTCCTGCACGCAGTGAACGCACAAG
TCTGATGGCATCACGGCGATCGGTTTTAACCCGCTCCCCCGGTTTTTTCGGGATAAGTGAGGGGGCGCA
GACCATGCAGTCAAAACCGGATTTCACCAGCCGACGATAAAGTCCATAACCACAGGGCCCCGCTTCATA
AACGATACTGACCTGCGATGACTTTGACCTGAGACGTTTACAAAGATTCTGAATATCAGTAGGTGATGT
GCCAATTTTACCCATCAGTTCAACAGGTTCTGAATTGATAGCATAAGCGACAGTAATCGATTCTTTGTGA
ACATCTAACCCGACGTACAATGTGTTATTGTTGTTCATGCTGGCCTCCGTGGTGGAAATACCGGGGTGT
AGCAACAGCTCACCCATGCGGCTCTGACAACGATGTTAACCCGCGTTAAATATCCCACGTTGGCCAGCA
TCTGTCTCACGGAAAGTCATACTGCCTTATAAGGCTTTCCAGTTGCAACTGCCCGGCGAGCGCCATCAG
TCGTTGATGTTGCAGTTCCATCATCACGCCACTCCTCTGCAGAATGAGTCGTAGATGGAGAGTGGATGA
TGCTGGGGGGTGCCCCTATTAAGTTGTCAAGCATGTTATGACCCCTGCGGGGTATAAAAAGTCCCGTCG
CGCATCATGGCGAACAGAACGTCGCAGCGTCGTCTCGCCAGGGCGATAAGCGCCTGATTGTGTCGTTT
TCCCTGACTCATTTTGCGGGTGTAGTAAGCCCTGGAGAGCGGATCCCTGAGCGCGGCGAAGGCCGACA
GGAACAACGCCCGTTTGAGAGCTTTATTACCCCGTCGCGAGGGATGCTCACCGCGTATTGACGAGCCG
GATCGCCGAGTTACCGGCGCAAGGCCAGCATAAGCAGCGAGATGTGCGGCAGAGGCGAAGGCGCGG
CAGGCGACCTCGGTGAGGAGTCTGGCTGCGGTCCTGACACCGACTCCGGGCATACTGGTCAGGACCG
GGTAAAGAGGGTGAGCAAGAACTCGCTGTTCTACCTCAAGCGCCACCTCGTCTCTTTGCTTACGCAGCG
TGATGAGCTGGAGTGCCAGACGTGGCAGTACTACGGCAGCGGCATTCGTGCCGGGAACGACGACGGT
TTGTTCGGCCAGTGCCTGAGCTATGTCTGCTGCAAGGCGTTTACCCAGACGAGGCGCAAGTTTGCAGA
GCTGGGCTGCCAGCTTCTTCTCACCCAGCGAAGCGAGTTTTTCTGGTGAGGGATATCGCTGGAGAAGA
TCGAGTACCGCCGGGTGCTCAAGTCTCGGACCGAGAACGCGCTCCAGTGCCGGATGTATCTGGGTCAG
AAGGCCGCGGATACGGTTGCTGGCCTGCGTTGTCTGTGCGGCAAGATCATCATCGAAGCCGCAGAGCA
TGGAGAGTTCGGCGATTTGCTCGTCAGCCAGTTTCAGCGTGCGTAGCGCGTGAGGCAGGGTACGGGC
AGCTTCGGCAATGATGGCAGCGTCACGAGCATCAGTTTTAGCTTCACCGGCGTGTAAGTCGGCTATGC
GGCGCATGGCCAGTCCAGGGAGGTATCCGACAAGGACTCCTTCTGAGCGGGCAACGGCGACAGGTAA
CGCACCGATGGTAGCTGGCTGATCAACAACCAGCAGTATCTGACCATGTTGTTTCAGGTCAGATATTAG
CGACCTGAGTTTGTTTTCGTCGTTGGGTAATGCTTTATCGAACAGGCGTTTACCTGAACGATTAATGGC
AACAGCGTGATGCGTATCTTTACCGACATCAACGCCGATAAAGACCTGGACGGATTCGTAATCGCTGG
ATTCGGTCATTCTGTCTCCCTTGTATATGGGTTAACCAGATAACCACGGGGAGCAGGTACCGGCATCCA
CGTTACAGACGGTCCCGGCAAAAGTGCCTGACCTGACCCCTATTAGCGGTTACCAGCGCCCCACCAGAC
CCGGTGACATCACCCCCCGGATCATGGACGACTGGGGGCAGTAATCATGCCGGGTCTGGCTGGCTAAC
ACCCCATTATAAGGGGTACGAATAAAGTAACGGGGTTGTCGAAGCTCACCAGATTTTCACCAGGATGC
ACGTCATACTCTTTTTTCTCCGGAGGCAGTGCCAGCATGGACTGCTGCTCTTCGAGCCAGCGATCACAG
GGACGGGCCTGGATTGTTTCATGCTTTCGTTGGTTAGCGACATCGTGCAGCCAGCGCAGACCGTGGCG
GTTGGCTGTTTCAACATCGACAGTGCTCCCCATCGGGCGCAGGCGAGTCATTAGTGGGATGTAAAAAC
TGTTACGGGTGTACTGCACCATCCGTTCCACCTTACCTTTAGTCTGTGCCCTGAAGGGGCGACACAGTC
GGGGAGAGAAGCCCATCTCCTTGCCGAACTGCCACAGCGAAGGATGGAACCGGTGCTGACCGGTCTG
ATATGCGTCACGTTGCAGAACCACAGTTTTCATATTGTCATACAACACTTCGCGCGGCACACCACCAAA
GAAGCGGAACGCATTACGATGGCAGGTCTCCAGCGTGTCATAACGCATATTGTCAGTGAATTCGATGT
ACAACATTCGGCTGTATCCGGGAACAGCAACGAACACGTGAAGCGGTGAGCGACCATTACGCATAGTG
CCCCAGTCAACCTGCATCTGTCGTCCGGGTTCAGTTTCGAACCGAACGGCAGGCTCCTGCTCCTGAGGA
ACCGAGAGAGAACGAATGAATGCCCTGAGAATGGTCATTCCGCCACGATATCCCTGGTCTCTGATCTCG
CGAGCGATTACCGTTGCCGGGATTTTGTAAGGATGAGCATCGGCGATGCGTTGACGAATATAATCCCG
GTATTCATCCAGGAGTGAAGCAACAGCAGGTCGCGGCGTATATTTTGGCGGCTCAGATTTTGCCTGCAA
ATAACGTTTAACGGTATTGCGGGAGAGCCCCAGTTCTCTGGCAATCGCCCGGCTACTCATTCCCTGCTT
GTGCAGGATTTTAATTTCCATAACTGTCTCAAAAGTGACCATAAGCTCTCCTGAATCAGGAGAGCAGAT
TACCCCCTGGATCTGATTTCAGGCGTTGGGTGTGGATCACTATTGCACCGTTCGTGACAGTGTTGTTGC
GCCTTGCTGCCAACGAAGCTCCACTGCCCATCAAGTTCACAGATAAGGGCAACATCAGCGTGAGCGAC
TGGCGCAGATGTTATCCGCCGGGGGGGGAGCTTTTTAATGTCCGTATGACKGTATTRATGCCGATTTTC
AGCGTACGGGCAGTATCGCGAACYCCGGCACCGTTGAAAGYCAWTWMMRYRATTTGGTYTTTAACTC
CCGGTTTACGGGSCTCGTAACAGTMASTGAGCTGAAAYACCCGGTGACAGTCACGGCMRCGGAATCG
MTCATGTCYTTTTGGGTTCTGCCCGTGACGGTAAACCTGAACGGACTGGCAACGGGGGCAATGAARRT
WMMCTRTGACCATGAAASAACCTTAAAAGCGRGTATTATAWCCYCTAATTCAACTAATTGGAGGCATT
GTCATTITTAATCTATGGATACACTTAAAAATCCTCTCACCTTACGTTAAAAACCTTCTCATGTTTAGAGT
TTTTTATTATGTTTATCATAATGATATGACGATCTGAGTTTTTAAAATCAAGAAGAAAAGATAAGTTTTTT
AAATGCACAGTGCCTGTGGATAACTTTGCGTAAATCCGGAGGACAGATCACTCTGAAGCAAGACAACA
GCATCTGAAAACAATATGCTCACGTTTCAACCACAGGGCGTAAGTGATCCTACCCACGTAATATGGACA
CAGGCCTAAGCGAGGTTCTGGTTTTCAAATTGTTCCGGACTGAGGCCGCCACACCAACTGTGCCGTCGC
CAGCGATTGTAATCACATTCGATATAATTAAACACTGTTGCCCGCATTATTTCCCGGCTGATAAAGTGTT
CTCCGTGGAGACATTCCACTTTCAGCGAATGAAAGAAGCTTTCCACGCAGGCATTATCGTAGCAGCAGC
CCTTTGCGCTCATACTGCCACGCAGATTATGCCACTTCAGCAGCGCCTGATAATCTGCTGAACAGTACTG
ACTTCCTCGGTCCGAGTGAACGATAACGTTCCGGGGTCTCTTACGCCGCCACAGCGCCATCTGCAGGGC
ATCGCAGGCCAGTTGCGCCGTCAGGCGTGGTGACATGGACCAGCCAATAACGGCACGTGACCACAGG
TCAATGACCACAGCCAGATACAGCCAGCCTTCATCTGTACGTAAGTACGTGATGTCTCCCGCCCACTTCT
GGTTCGGACCACTGGCGTAAAAATCCTGCTCCAGCAGATTTTCTGACACAGGCAGGCCATGTGCGCGG
TAACTGACCGGGCTGAACTTCCGGGAGGCCTTTGCCCTCAGCCCCTGACGGCGCAGGCTTGCCGCCAC
GGTTTTTACGTTAAAGTGGTAACCCTGAGCACGCAGTTCATCCGTCAGGCGTGGTGCACCGTAACGCTG
TTTTGACCGGGTAAAGGCCGCCGCGAGGACAACGCTGTCGCAGTGCTGACGGAACTGCTGACGAGGG
CTTATCCCTGTCCGCCGCTGACACCACGTATACCAGCCGCTGCGGGCCACCCGGAGCACGCGGCACATT
GCCTTGATGCTGAACTCAGCCTGATGTTTTTCAATAAAGACATACTTCATTTCAGGCGCTTCGCAAAGTA
TGTCGCGGCCTTTTGGAGGATAGCCAGTTCCTCTGCCTGCTCCGCCAGTTGCCGCTTCAGGCGGGCATT
TTCAGCGGCCAGTTCACTTTCACGCTCAGACGAGGTCATCTGTTGCTGCAGTTTGCTGCGCCACGCATA
GAGCTGAGATTCATACAGGCTGAGTTCACGGGCTGCGGCAGCAACACCGATGCGTTCAGCAAGCTTCA
GGGCTTCACTGCGAAATTCAGGCGAATGCTGTTTACGTGTTTTTTTGCTGGTTGATACTGTTTTTGTCAT
GTGAGTCACCTCTGACTGAGAGTTTACTCACTTAGCCGCGTGTCCACTATTGCTGGGTAAGATCAATTT
GCTCACTGGTATTTACTACGTGCATCGAATGAGTGATCAAGAGCCGTATGACGCGAGAGTGTCACGTA
CGGTTCCGTGAGCAGCTGGAGAGTGAAACTCTCTCCGGCTGACTCGACTCGCAGGTTCCGACAGCGGT
GACGAACATGCAGCGGTGTTGTTCTCGCTGATCGGCACACGCCGTCTGAACAATGTGGAGCCAGAAAA
ATGGCTGCGTTACGTTATTGAACATATCCAGGACTGACCGGCAAACCGGGTACGCGATCTGTTGCCTTG
GAAAGTTGATCTGACTTCTCAGTAAATATCAATATGATTCTGACGAGCCGCTTACCTTTGAGCAGAGAA
AGGGGAGAGGGATCCCACATCCATGAGCTAGGAAAATACAAAAACTGGCTATACAACAATTCAATATA
TTAGGACTTACTTGCGAGGAGAAATATAGCACCGCTCAGGCAATTCCTGGGTATTTACTTTTCTTAGTTT
CTTTACTCGGGATGTTCTGTCATCAGAAAGGCGATTCTAAATTTGTAATATAGTCACTCATATTCCCACG
TAATTTCTTTAGATCAATATATTGCACATTTTTCTCAAAAAACTGATCGGAAATAAGATTTGCAACATTGC
TTGTTGTATCATTTTTAACGCTTCTATTCATCCCTTCACAAAGTTCGTCCATTTTCTTTTTGAAATAACTGC
TTCCTGATACAAGATGATTTATGGAACTTTGTGCAGCATTGATGTCTAGAGAAATGCGATAATCTTTCTC
AACCTGTTTAAATATCTGTTTACGGAGGTTATCGCCGATTTCTCGGCTGTGTTCTGCACAAAACTTATCA
ATACATTGGTTTACCACCCGGGATACGTAGACTTTTTTTTCATCATTTTTCCAATCTGTAATATTTTCACC
GATTTGTTCTGAAACAGAGCTCAAAATACTAGTATTTATTTTGGTTGATGATATTTTTACTATCCTTTTCC
CCATTGTACAGCGGATAATCTCATCCACCTTTCCTGAAAAATATCTTTTGTGAGATAAACTGATTCCAAA
ATTATTAAAAGATGTTCCAAGCATTATAATCACTTTGTCATGTAAAAATAGTAACATTGTCATTATTTTAG
CTGATTCAAACAATTCTGTAAATGCTTTGAGTTCTTTACTCCCTTGGCTCAATGATGGACCGTCTGGAAT
CCCAGAGACTAAACTCTCTGAGAAAGAGAGTATGAAAAAACTTGGGGCAGTCCCTAAATGAAGGGACC
AACAGCGGTAAAATAACCGGCATCATTCATAATGATGCCCTGTTAGTTGTAGTTATGAAGATTTACGTC
ATTAAAATTGCTGTTCGTAGTGTCAGTCCTATGGTCTGGCATCAGCTGAGGATCGTAGCCGATACATCA
CTGGCTTCACGACATTCGTGTTGAGGCTATTCATGAAAACTCCACGCTGAAAACGCCGTTTTGTATAAAC
GGTCACGGTATGCCCGGAGCCACTGTTGCTGATGAATTCGATAAAACCTTGGCGTTTCTTGAAGCTATC
GTTAATGCGGATAATGAAACAACGATTGGTGAGATTCGCTCTTTTGCCGACGCCCTGGACGCTGTCCGG
TTCAATCGCAACAAAATCAACCGGCAGCTGAGCAAACCCAACCTTGCCTCTCTGGCGCTGGAGCATGAA
GTGATTTGGCTGGGACGTAGCCGCTGACAGCTTCTATAAAAGTATGATCCTACCCACGTAATATGAACA
CAGGCTTAAGTGAGGTTCTGGTTTTCAAATTGTTCAGGACTGAGACCTCCACACCAACTGTGCCGTCGT
CAGCGATTGTAATCGCACTTGATATAATTAAACACWRYMWMWWWMMWWWRWRRMRWMWM
MMRWMWRMRRRRRMWWWMWRMMWYMMRWMWWRRRMMMWMWRMWRMMRRMM
MWMWRYRMRRMWMMMMAMMYTCCTGTATTTCCCTTCCTCTATCACAGTGCACTTTACTTAATAG
GGTGGGATGTGTTCATTACATTTATTGTGTTTTTTATGATTGTTTTTGCGTCCAGTGTCGTTCGTCATGG
GAAACTGGTTCTATGGGTAGTATCGCTCAGTCTAGTTAACTGTTTGTTTCTTTTTAGAAATTAAGCAGAT
CACACAATTCGGGGAGAGATCCTTCTAAGTGCAATTCTTTGCAGCTATTTAGCACACTTAAAACAACTCT
TGTATTTTGTCAGAAATAATATTAAACATGTCTTGTTCATTATTGTTAAGTCTGTCTGCTATTTCTGTAAG
TTTTATCCCACTATGATGGCATATATTTATATATGCCATCATAGCCTCGATATTCCCTTTCCCTGCGGCCA
GAAATAATCCTGGTAAACCCTCATTATCTCTTACATCCAATAGTTTAATCGTTTCTTTTGGTGATAAATCA
AAAGTTTTAATAAAATCTCCATAAGCTTGAATCGCAGCGGAATGACCTTCTTGAAGAGCTATGAACAAA
GCAGATTCCCCAGCATTATTTCGTGCTGCAAGAACTTCTAGTTTTTCCTCTTTGGGTATTTCGGCTGCCAA
CAGCGATTCACCGTATTTACGCGCTGCATCGGCATTTCCATGCCAAATCAATGAAAACAAATTATGGCG
GTTATACTCCTTATTATTCTTTGTCGGAATGGATATGCTCTGTATTTTGGCCAATTTTCTGAAAGAAACAC
CACTATATCCCCCCCCTGATTTTTCTTCATTGATGGCGTGGGATATAGCGGAATTTGTCACGTTAATGAT
ATTACTTTTCATTCCTTTATTTTCATTAGCAGTATTGCTGCCTATTATTAGGTGCATATTTTTGTTGTTTGC
TGGATGTAATCCATAGTTATTTATTGACATTTTTGTTCATCCTTTTTGCTAAAAAAAACAATAACCTATTTT
TGTAATGTAATAGATATTAGTCTATTGCCAATCTAAAAAAACCATTGCCACATTGAATGGCTATATTCAC
CAATAAATTCTTTCGATTTCGTATATTGACATATCGTAGATATATCTTATCCCTAAGATTCAATAATCGAG
TCCGTTTACCATTCTGTGCAAGCGCCTTCACTCAGAAGTGACCGTCAAGGCGATCACCAAACTTGATAAT
AAAGCAGCTCATTGCCATACGCCAGTCCTTAAGGGGCGTTGTCCATTTCTGTGATGCTGCCTAGATTTAA
CTGCCAGCTCCGGCTTATCTACATCTAAATATGTGAGCCTAATACCGCTACACTTTGGCCAGACTATGTT
TTTCTCCGGGAAGTGGGCTAACCGTCTCCATAAAGTGACGAACTTTTTTCAACAGTGGCCATATTGAAC
GGCACTGATGATTACGGGTTATTGTCTCATGAAGAGCCGGCCACATCACTGACAGCAGTGACTTCAGG
CCTTCAGCATCAGACAGCCTGAACCAGTTAATCCATCGCCCGACAGATTTAGTAATCTAGGGCTGGTTTT
TCTTCAGCTGTTGCGGTTCGAAAGTGCCATTACGATCACGCAGAGTACGGATTTCCAGAGGTTCATCTC
CAGTAGTGATAATCTTCGTTAAGCAACCATTGCGGGAATTGCTGCCAGATCTAAGCTGATTTTTCTCGCA
CCCAAGATGATGAGTCATATTAGCATTGAGTACGGTTTCATTTTCAGCATCCAATCAAACTGACTGAGAT
CTTCAGGATTTTTGAGACTTTTGGCTAGTTCATTAGCTAAAGCCCGCAACTTATTAGATGTCAACTGTTTT
TCATTCATAAATTCACCTTCATTTGATATCAAATTAAATAGTTCAACATCAGAGAATTATACAAACTTATG
TACAGGCTCCTATGATCTTCGAATCCTGAAGATTATAGTGAAAATAAGTTTTCACGTTGAAATCCCACAT
AGTTAGTATATGATAAATAATCATGGATGACCTGATTAACCTCTTGGGGTAGTGCTITTATAGAAGAAT
AAATATTCTACTAATTTAATACATCACCTAAAATAGTATTGTTTTTTATGGTTGGTTGAAGGTCGTGGTG
ATGATGTTAGTCCTTTTGAGAATATTCCAGTGCCAGATCTTACTTATAATAAAGGGGCTTGGCAGTTTGG
ATAAACTATTGTTCTTATTCCACTAATACAAACTTTTTGTAAAGTATATTAAATATACTTCATTTCATACTG
AAATCCCCGGAAGTATTCAATCAAACAATTACACCATAAAAGGAAGAGGGGGCCCTAATTAAGTTGTC
AAGCATGTTTATGATACCTGCAGGGTATAAATTTTCTGGCTGCAAGCAGACACATATAAAACGTCTTTAT
TGATCCTACCCACATAATATGGACACAGTCGTAAGTGAGGCTCTGGTTTTCAAATTGTTCCGGACTGAG
ACCRCCACACCAACKGTRCCRTCGCCAGCGATTGTAATCGCACTCGATATAACTAAACACTGTTGCCCGC
ACTATTTCCCGATGGATAATAGTGTTCTCCGTGGAGACATTCCATTTTCAGCGAATGAATTTTCATCTGT
TGCTGTATCCAGTCATACAGTGACTGCATCAGTGGCGCGGCTCTGGCTTTTCTTGCCGCCAGACGCTGT
TCTGCTGAACATCCCCGGACTTCTGCCTCGATGGCATACAGTTCACCGATACGCTGCTGGGCTTCCGTTA
TTCTGCCGGATTCGTACAACGCCCGGTAACTACCGTAAGCATCGGCCTGAAGCACTCCGCTGTAACCGG
CCAGATGATTCTGTGGATGGATACCTTTCCGGTCCGGTGAGTACGCGAACCAGACCGCCGGAGGCATT
TCCGAACCGGCGTTACGGTCATCACGAACGTAGACCCACAGCCGGGCAGTCCTTGTTTTACCGCTGCCC
GGCTCCTGGACCGGAACGGGGATATCATCGGCATGGACTTTACCGGGCATCAGCACATACTGGCGCAG
GATGTCATACAGCGGTTCCAGCAGTTCAGCAACAGCACCTGTCCAGCGCCCCAGTGTGGCACGGCTCA
GCTCCACGCCCTGACGACGGTATATTTCTGACTGGCGGTATAACGGCAGATAGTCTGCATATTTTCCAG
CGACAACATGGGCCAGAAGCCCCGCTCCGGCATAACTGCGTGCAATGGGTTTTGAAGGCACCGGTGCC
TGCACGATATGGTCACACCGGCAACAGGCCAGTTTCGGACGTTGTGTTTCGATAACCTTAAAGGCGCTG
CTGATAAGCTCCAGCTGCTCTAACACATCACATCCCAGAGAACTGAGTTCGCCACCACAGGCCGGGCAG
CATTCCTCTTCCGGACGGATGACCCGGGTTTCACGGGGAAGTGAGGCAGGTAACGGTTTATGGGCTGA
AGACTGGCGCAGGGGAGATGGCAGTACCGGGTCATATTGCTCACCCAGCGTTTCCGCCATTTCTTCCTG
AAGTGCGCTGATTCGCTCCTGTGCTTCCTGTATCTGCCGTTCGGTTTTTGCGCGAAGTTTTTCTGAGCTTT
TACCGAACTGCATGCGCTGCAGTTTCGCAACCAGCGCCTTCAGCCGGTTGATTTCGGAAGCATAAGCCG
CCACCCGCTGTGAGAGCAGGCGGTTGTATTTCGCCATCTGGCGGATGGTGTCCTGTTGCGTCTGCAACA
GTGCCCGCAGGCGGGCATTCTCATGAGCAAGTGAGGTGTCCATATCCTCACTTTACAACGGGTTATATG
CGGATGCCAGCGCGTTCCGTTCGTTTCGGGTGTTTCCAGTCGATGCCCTCAAGAAGCATGGATAACTGG
GCCGGAGTAAGGTGCACCTTGCCGTCACGGGTGACCGGCCAGACGAAGCGGCCCCGCTCCAGGCGTC
TGGTAAAGAGGCACAGTCCGTCACTGTCAGCCCACAACACTTTTATCTGGTCACCCCGGCGTCCGCGGA
AGATGAACAGATGCCCGGAGAACGGGTCATCCTTCAGGACGTTCTGAACTTTTGAGGCCAGGCCGTTA
AAGCCATTTCGCATATCGGTGATACCTGCAACCAGCCAGATACGCGAACCTGCAGGGAAAGATATCAT
CAGTGGCTGCTCCCTTTTATTTCGCGGATAAGTATCTGTAATAACGCCGGAGTCAGTTTACCTTTAAGCC
TGAGGAAAGGGAGCGCAATATGAAGAGCTCAGAAAACTGGGTAAAGGCGATCATCTGGTGAAGCTGA
AAACCAGCCCGCAGGCACGAAAAAAGTGGCCGGGACTGGGAAATGAAGTGACAGCCCGCCTGCTGAC
CGTGACGCGCAAAGGAAAAGTCTGCCATCTGCTGACGTCGATGACGGACGCCATGCGCTTCCCCGGAG
GAGAAATGGCGGATCTGTACAGTCATCGCTGGGAAATCGAACTGGGATACAGGGAGATAAAACAGAC
GATGCAACTGAGCAGGCTGACGCTGAGAAGTAAAAAGCCGGAGCTTGTGGAGCAAGAGCTGTGGGGT
GTCTTACTGGCTTATAATCTGGTGAGATATCAGATGATTAAAATGGCGGAACATCTGAAAGGTTACTGG
CCGAATCAACTGAGTTTCTCAGAATCATGCGGAATGGTGATGAGAATGCTGATGACATTGCAGGGCGC
TTCACCGGGACGTATACCGGAGCTGATGCGCGATCTTGCAAGTATGGGACAACTTGTGAAATTACCGA
CGAGAAGGGAAAGGGCCTTCCCGAGAGTGGTAAAGGAGAGGCCCTGGAAATACCCCACAGCCCCGAA
AAAGAGCCAGTCAGTTGCTTAACTGACTGGCATTAAGGCCGCCGGGGACGTTTTTTTACCACCGGGAG
TTTGGGCATAACGGTGCTTTCAGTAAGTACGACAGGTACTGTGGAGGCACTTCCGTAAAGAAGGGCAT
CAAGTTCCTGCTCCGACATACCTGCGGGCAGAGGCCATGAAAGGCCAGCGTTGCGGAAGCGCACGAA
CATACCACAGACAGTTGATTTTGGTACGCCCAGGCGACGTCCGGCCACAACCCGTGGTAAATGTTCTTC
GAAATGAAGGCGTAAAGCTTCAGTGATCCAGGTCCGGTATTCCATACGATAGTGTCCACTTCAAAATGA
TGGACATTATTTTTGTGGAGCCGGAGGAAACAGACCAGACGGTTTAAATGAGCCGGTTACGAAGAAGC
TGAGGTTCTGCGCTTTGATAATTTTGACGATGCCGTTGAGTGTTGCGCAGACCTGAATATTCCGTTCTTT
GTGGATGCCGGAAACAAAAAACTGGTCTTCTGGTTTGTACGTGTTGATGACGAAGGGTATCCGGAAAT
TGCCCGCTGCATGGAGCGGGAATTTGCGACAATTCCTGCCGGTATCAGCGCCGACGGCATGTATTGCC
CTGAATGCGGTACGGTTCACTGGCCGGATGGCGTTATTCCGCCTTTCTGATGCTTCCCCGTTTTGCCGAT
ATTTTTCAGCAGGGAAACCGATGGCTTAACTGGCTGGAGAAGCAGTCGGTGCAGATGAGCCGCCTGGA
ACATTATGCCGGTCAGGATGAAATTGGCCTGCGTTACAACAGCCACCGGACAAAACGAGAAGAAAACC
TTGTGATGAGTGGCGACGAGTTTATGGAAAGGTTCTCCTGGCATGTGGCGGATAAGGGGTTCCGGATG
GTGATACGGGGTCCTGAGTCCGGTGAAGCGGCGATTACTGGAAGATGTGGTGTACGCCATAACGGAG
ACAGTGAGAAAAACGGCGAGGCAAATCACAAGGAGAGGGATGTATCAGCAGTGACTGAAGGTTGACC
CGCTGAAGTGCGAGCTGTGCGGAGGTCAGATGCGGTTTACGGGGCTGAAGAGGGGTTACCGTCTGGC
AGAGCAGGTTATGATGCATGAGCCGCTGGCTCGAATACGGTGGTGCGGCTGAGAGTCGCAGAGGGGA
AGTTGCGTCCATGTTGAGGGGAACGGAGCAAAAAACCATCAGAGATACATTGAATCAATCAATGTAAG
CAATATTAAGTGTCTATACCGATTACTTGATGATCAGGTTGTGTTGAATAAACGCGCATTCTTCGGTGCT
TTTTTAATCTTTTGGGGATTTAAATTCCTATCGATGAACTGTCGATATGAAAAAGCGAGCATCATACTGA
CGTCAAACAAAGGGGTCGCAGACTGGGGAGAAATGTTCGGAGATCACGTGCTGGCAACAGCGATCCT
AAATAGCTGCGCTTAATACCGCTACACTTTTGCCAGCCCATGTTTGCCTCCGGGGAATGGGCTGACGGT
TTCCATAAAATGGCGAACTTTTTTCAACAGTTGCCACATTGAGCTGCACTGATGATTACGCGTTATTGTG
TCGTGAAGTGCCTGCCATAGCCGTTCAACATGATTCACCCATGGCGAGTAAACCGGCTGATAAATGACC
CTGAACTTCGGGTTCTCCTTCAGCCAGCTCTGTGTTTCCCGGCTTTTGTGGATAATGTAGTTGTCCACGA
TCAGCGTGATGGTTTTCGCCCGACGGTATGTCGCTTTAAGCCGCTTCAGCAGGCTGATGAACAGCGCCG
AACTTTTGCTGTTGCCGCCCACACAGCTGACTTTACCTGTCCCGCTGTGCAGCGCTCCGGCCAGATAATA
TTTTTCATTCTGTCCCGGCGTGACCACCCGTTTTTGCTGTCCGCGCAGTTGCCAGTCCGCACCGATTTTG
GGATTAAGATGGATATCCACTTCATCTTCATAAAAGACCGGATGCTCTGCGCTGCATTCGTCCAGTGCTT
TATGGATTGCTGCCATCTTTTCATCTTTATGCGGGTCACGGATACGCAGAGTTGGCGCAGCCCTTCGCC
ACACAATCCCCGCAGACGGCAACCAGCGGCGAACGGTTCCGGCATTTAACTGGCAACCGGTTATCTCAT
TGATTTTTATTGCCAGCAGTTCTGTACTCCAGCGTGAACGCTGGTAGCCAAAGTCGCCGGGAGAATGTT
TTACCAGCTCACGTAACAGTGTGCAGATATGCTCAAACGGCCAGCGACGGGCACGCCCGGCAGGTAAT
GATTTCAGTCCCTCAACACCCGACTGCGTGAACCAGTTAATCCAGCGTCCAACAGAGGAACGGGCGCA
GCAGAGCGTTCTGGCAACGTCGCTGACACGGTCGCCCCGGTGCAGCATCAGCATGGCAGTCAGTCTGC
GGGCATAATTTTTATCGTGTGTTTTATGGATGGCTTTCTGCATCAGGCGTCGTTCGTCACGGGAAATTG
GTGCTATGATCGGCATTGCTCAGTCCGGTTGGTGATTTGTTTTGATTTGGCGATTGATCAGATCGCACA
ATCCGGGCTAAGTTCCCTCAAAGTGATCTACTATTCCGCGCAGCTATTTAGATCGRMWRCWMMMMR
MTGAMTATMMMMRRARARARWWACMGRTWMMAAGAGAAACGTAAAGCTGGAGTGCTGACCAA
AAACACAACGCCAATCAGTGATGATGAAATGGTGGAAAACGGACARCATCAGTMACRMMMRTMTCT
TAGCAGGCATRMRAATCGTCCGCATCCCCTAAACGGTCAMMATGCTTCTGCCGTGAAGGAGGGAGAA
TGCCATCTCATCACTTATATCTGGCGAATGAAACACAGATGGTCGGCCTTGCCCCCAAACACACGGCGG
TGCAAAATGGAATGGCAGAAAACTTCGTCAAAACGATGAAATATGATTAAATCAGTGTTATGTTCGAAC
CTGACGTGTTAACAGCAGTAAAGTAGTCTCGCGAAGGTGTTCAAACATTATAATGAATGGCATCCTCAT
AGTGCGCAGGGTTATCGCTTGCTGCGGAAATATCTGAAGCGACGTACCAGTAATGGGGAAAATGATAG
CTGGTTTCTGAAAATATAGAGGAAAATGTGATATATATATCCTGTTAATATTTAAGGAGAATATAAAAT
AAAATGAAAGTCTCGTTTAAGTCTTTAGGCTATATTTTTCATGATATATATAATAAAAAACACACTATCG
ATGAGTTCAACGATGTGGTAAGAAAAGCAGTTTTATCAGGTAAAATTAACGAGCTGAATGCATGTCAT
AAAGTGGCAATTTTTCTTGCCGAAAAAGACAATGAGATAACAAAGAAAGACAAAGCCAAGATAATAGA
TACATTAACTGAAAACTATTCCATTGAATTTCAACAATTAATGAATATTAGTGAGCGCACGTTAAATTCA
TCTTTATATATTACTCCTGGTGAAAGTGGTTTTGTCAGTTTTGTTAATCGTGAAGGAAAAATATGTCATA
CTGCATATGTAAAATCATCAGATAATAGCATGGCATATTACCATGCAAATTATAGCAGCATTGACAAAT
ATATTACGGATATGTGCGGCCTTATTTGTATGAGGCATATTGAGTCTACTGGCATTATTTTTTATATGCTT
GATGAAAAAGTTTTATCTGCCATTGCTGAATTTATGAATGAAAAAGGATGGCGGGCTGCGTTTTGTTCA
GCAAAGAACTTATATAAATGTGTTTAATTTTAGAAAAACTATTTGGTAAAATCCAACAAGAGTCGAGAG
GTTCGCGTTGCTTCAGCAGGGCCTCAGCCTGTTCAACCAGACTCAGTTGCAGGTCGTGACGCAGATTTA
GTGGTGGTGGTTGATTGTCAGATACCAGCCGATGATCTTGTAGTGCATATCATCAGACTTTGAGAAGCA
TATGGTTTTGCGGGTAAGCCGCTTAAGATGTGTCCGCAGGTTCAGATTATGTCGCTCTATCCGCTGAGT
GCATTTCTTGCTTATAACATGACTGGTTGAGGACAATAAAGTTCGGTATACCGGCTCAGGCATCGGTCA
TGTAGAAGGCAATGCTAAACTGGCTCAGCAAACTAAGAGCAGCGCGCATCCACTTACCCAATGCGGTT
GAGAAAATAGCCTTCGATCGCTTTCATGTGGCGAAACAACCGGGTGAGGTGGTGGATAAAACCCGCCA
GAATGAACATCCGCATCTTCCTGTTGAAAGTCGTCGTCAGGCAAAGGGAACTCGCTTCCTGTGGCAGCA
CAGCAATAAATGGATGACCGAATCCAGGCAGGAAAAGCTGATATGGCTGCGTGCGCAGATGAAGCTG
ACGAGCCTGTGCTGGGCGCTGAAAGAACTGGCAAAGGATATCTGGAGCAGACCATGGAGCGAGGAAA
GACGCAACGACTGGCAGAGATGGCTGTCGCTGGCGGCCAACAGTGACGTCCCCATGATGAAGAATGT
GGCTAAAACGATAGGGAAAAGGTTGTACGGTATCCTGAACGCCATGCGCCATGGCGTATCAAACGGA
AATGCGGAAGCACTGAACAGCAAGATCAGGCTGCTGAGAATAAAAGCCAAGGGCTACCGCAACCGGG
AGCGCTTTAAGCTGGGAGTGATGTTCCACTATGGGAAACTGAATATGGCGTTCTAAGCCTTCCCACCAT
GATCGGAGAAGACCCTTACGCTTACATACATTACATATAATGTATCGTTTTATAAAAGATGATAGACAA
TCAATATAAGATAATATATCTATTTTATAGAGGACGTTTTCTATGCCCATAAAAAAGCCCTGTCTAAAAC
TAAATTTAGATTCTTTGAATGTTGTGAGGTCTGAAATACCGCAGATGCTTTCTGCGAATGAAAGATTGA
AAAATAACTTCAATATATTGTACAACCAAATACGGCAATACCCAGCGTACTATTTCAAAGTAGCATCGA
ACGTGCCAAATTACTCTGATATATGTCAATTTTTTTCTGTCATGTACCAAGGATTCCAGATTGTGAACCAT
AGTGGAGATGTGTTTATTCATGCATGTCGTGAAAATCCTCAGAGTAAGGGGGATTTTGTTGGGGACAA
GTTTCATATTAGTATAGCTAGGGAGCAAGTTCCTCTGGCATTCCAAATTTTGTCTGGTTTATTATTTTCAG
AGGACAGTCCTATAGATAAATGGAAGATAACTGATATGAATCGAGTTTCTCAGCAATCTCGTGTGGGG
ATAGGAGCTCAGTTTACGCTATATGTAAAATCAGATCAGGAATGCTCGCAATATAGTGCTTTATTACTTC
ATAAAATACGACAATTTATAATGTGTCTCGAGTCTAATCTATTAAGAAGCAAAATCGCTCCCGGGGAGT
ATCCGGCGTCAGATGTTAGACCTGAAGACTGGAAATATGTCAGCTATCGTAATGAACTACGAAGCGAT
CGAGATGGAAGTGAAAGGCAAGAGCAAATGTTACGAGAGGAACCATTTTATCGTTTGATGATAGAGTA
GAGAAGATGCCAGCTACACCACCTTGTAGCTTGAAAACTGGTTAGAATAACTACGATTAGATTTGGCAC
TGCGTATGCAGCCTGATTCCTGCCCGTGACAGAGCATTCGGCTCATAAACTGCTGAGCTACTCATCTGA
AATCCGAACAGTTGTCCACCACTTCAGCGTGCATCTCCTGTTTGAGTCGTTTGATTGCTTTTCGGGATAT
TAACCTGTCGGATTTTGCATTAGTTAATGCTCCCATCAGGCCTTCATCTTTTCCAGTGCCTATTTTCCTGTT
CTCCTTTTGCACAATATAATCAGATCATCTGTATCGTTGACGAGCTCGCTATCTTATAGGAGCATTCTGA
ATAAGCTACCTCCTTGTAAGGAGGCTTTTACTATTAGTAACTGCAATCTAATAGTATTATAACATCTCTAT
GGTTGTCTTCTATGCCGTTAAATAAAACATTTTCATCTAGTATATTTTCAACAAAAAATTCGTTATCAACA
GATATGTCTGTTAACAGAGATAATAGAACAATAACAAGTTCTATTATGAGGGTCTCAAACTCCAGTGAA
CTAATTCAGTTTAAGAATAAAACAGCACCTTATTTTTCAGAAAAGAGAAATGTGAAGGTAAATATAAAT
GGAGTGGCAAAAGATATATATGGAAGACAAATAGTTTGCCGTCACCTAGCTTCCTATTGGGAAATGAA
TTTTATGGAAACAAATGGGAAGGTAAACTATCAGCTTCTTAGCACCCCTGATGCTATTGCAAAAAATGT
ATGTTTGGAAAAAACGGAAGATTTTTCAAAATCACCAGCTTATATTTATTTTGTGGAAAATAAAAAGTG
GGGAACAGTTATTACTAATTTTTTTTATAACATGAAAAAAAATGGAGACTTTGTCAGAACATTATCAGCA
TGCACATTAAACCACCAAATGGCGTTAGGGTTGAAAATAAAGCGCGTACAAGAAAGTGAAAAATGGGT
TGTTCAGTTTTTCGATCCTAATCGAACTGTTACTCATAAACGTACTGTATTTACTTGTGATAGCCATTTCG
AACTGAGTCAATTATCCGCTAAGGATTTTTTTGATGATTTTTATTGGAAAATTTATGGTCTGGAGCAACC
CGGGCAGGTAATATTTGAAGATAGGCATAATTCCCCTCTAACAAACACAGTGAAACTTTTACCAGATGA
ATTGATCAATTCCAGAGTTATATATCATGCGATAACAAAGAATCTGACAGAAGTATTATTTATACTGATG
GAAAAATACAAAAATGGTGAAATTTCTCAATCTAAACTGGTGAACCTTCTGGCCACGAGAAGCTCTGAT
GGTACTCCTGCATTTTATATAGCACTTCAAAACGGATGTTCAGACATTATTCAAGTATATGGTAAGATAT
TAAATATGTGCAATCTTAGTCAAGAAACAATACTTACATTGCTTGCTGCGGTAGGGGCAAACAATGTGC
CGGGTTTATGCATGTCTTTTATGAACGGACATGTAGACACAATAAAAGCCTATGGGGAAATAGTCTTTA
AAACCCCGTTAACTTCCGATAAGAGATTATATCTATTAGCTGCGAAAGACAGCCATGATTTACCTGGGT
TATTTTTTGCATTGCAGAATGGACATGCAGATAGTATTCGTATGTTCGGAAGTCTGCTTAATAAGAAAAT
GCTTAGTAGTGAACAAATTAAAGAATTATTGAAAGTAAAGCATGGGTTGTTTATGGCACTTCAAAATGG
GCACACAAAGGCCATTATGGCGTATGGAGATATACTAAAAATACTCCCCCCTCACCAGGAATATATAGA
TGAATTATTATGGATAAAAAATCCCAACGGTACATCAGGACTTTTTATGGCATTTTATAATGGCCATACC
GAAACAATACGTGCCTTTTGTAATATTCTAAAAAACTATAGTTTTACCACAAGACGATTGGTCGAAATGC
TGTCCGCAACTAATAAAGATGGTATTCCTGGTGTATTTGTATCTGTAGTAAATAGAGACAAAGAAACAA
TATTAGAGTATTGTAGAATAATAAAAGAGAACAATCTGGAACCGGATACTATAGCAGAACAATTTTCAA
AGAAGATGAAAAAAACGTTTATTGAAATAATAAATAGATTTAATCATTTTTTATGAGACTGTAATTTAAA
TTGTGTAATTACCTGTTTTTGATATGTTCACTCCAACAAGGTGTAGTGACCGGGTAACTGCCAACTTTCA
GGCAACCACCTCGATTAGCAACCTTATTTCCATATCGAGTTCAAATATCCCGTAGGGGTTTACATGGGC
GTAAACTGATGATTGTTCAGGTTTCCATGGTGTGGCCAAATCTTCTTCATGGATAAAGGTCACATCCAG
AGGGTGAGTATGTTCCAATATTTAATACTAAAGATATGAGGCTTGGAATTGGGTTGCATCTTATTGATTT
TATAAGAAAGAGTAAAGATCAAGGATTCCGAGAGTTTTGTTATAACAAAAACATTGATCCTGTTAGTCT
GGATAGAATTATAAACTTTGTGTTTCAGCTTGAGTATCATATACCAAGAATGCTAAGTACAGATAACTTC
AAAAAAATAAAACTCAGAGACATATCTTTGGAGGATGCGATTAAAGCATCTAATTATGAAGAGATTAAC
AATAAAGTGACTGATAAAAAAATGGCTCATCAGGCCCTTGCTTATTCTTTAGGCAATAAAAAAGCTGAT
ATAGCACTTTATTTACTATCTAAATTTAATTTTACAAAACAAGATGTCGCAGAGATGGAGAAAATGAAC
AACAATAGATATTGTAATCTGTATGATGTTGAATACTTACTTAGTAAAGATGGTGCCAACTATAAAGTG
CTAAATAGCTGCGCTTAATACCGCTACACTTTTGCCAGCCCATGTTTGCCTCCGGGGAATGGGCTGACG
GTTTCCATAAAATGGCGAACTTTTTTCAACAGTTGCCACATTGAGCTGCACTGATGATTACGCGTTATTG
TGTCGTGAAGTGCCTGCCATAGCCGTTCAACATGATTCATCCATGGCGAGTAAACCGGCTGATAAATGA
CCCTGAACTTCGGGTTCTCCTTCAGCCAGCTCTGTGTTTCCCGGCTTTTGTGGATAATGTAGTTGTCCAC
GATCAGCGTGATGGTTTTCGCCCGACGGTATGTCGCTTTAAGCCGCTTCAGCAGGCTGATGAACAGCGC
CGAACTTTTGCTGTTGCCGCCCACACAGCTGACTTTACCTGTCCCGCTGTGCAGCGCTCCGGCCAGATAA
TATTTTTCATTCTGTCCCGGCGTGACCACCCGTTTTTGCTGTCCGCGCAGTTGCCAGTCCGCACCGATTTT
GGGATTAAGATGGATATCCACTTCATCTTCATAAAAGACCGGATGCTCTGCGCTGCATTCGTCCAGTGC
TTTATGGATTGCTGCCATCTTTTCATCTTTATGCGGGTCACGGATACGCAGAGTTGGCGCAGCCCTTCGC
CACACAATCCCCGCAGACGGCAACCAGCGGCGAACGGTTCCGGCATTTAACTGGCAACCGGTTATCTC
ATTGATTTTTATTGCCAGCAGTTCTGTACTCCAGCGTGAACGCTGGTAGCCAAAGTCGCCGGGAGAATG
TTTTACCAGCTCACGTAACAGTGTGCAGATATGCTCAAACGGCCAGCGACGGGCACGCCCGGCAGGTA
ATGATTTCAGTCCCTCAACACCCGACTGCGTGAACCAGTTAATCCAGCGTCCAACAGAGGAACGGGCG
CAGCAGAGCGTTCTGGCAACGTCGCTGACACGGTCGCCCCGGTGCAGCATCAGCATGGCAGTCAGTCT
GCGGGCATAATTTTTATCGTGTGTTTTATGGATGGCTTTCTGCATCAGGCGTCGTTCGTCACGGGAAATT
GGTGCTATGATCGGCATTGCTCAGTCCGGTTGGTGATTTGTTTTGATTTGGCGATTGATCAGATCGCAC
AATCCGGGCTAAGTTCCCTCAAAGTGATCTACTATTCCGCGCAGCTATTTAGAATATTTTATCAATAATG
GATTGGTTGATGTAAACAAAAAATTTCAAAAAGCAAATAGTGGGGACACTATGCTTGACAATGCAATG
AAAAGCAAAGATTCAAAAATGATTGATTTTTTATTAAAAAATGGAGCGGTATCAGGCAAACGATTTGAG
AGGTGACCTAAAATATATTTAGGTTGTGCCCTGAATCTGAAGTTGTCATTTCTGTTATATTGATCGACAT
AGGGGATGGTGCAGACCGTTCCCCTGCAACACCACATTGCATGTGGTGAACGGAACCGCCCCGAGTTT
CCTAGAGAATGTTTTATCTGTGAACTCAGGTTGCCAGATCATCGTTTCCGATGGCAGCATAATAAGCTTT
TTCTGCTTCTGCCGGAGGGATATGTCCCAGCCTTTCCAGTAATCCCTTAAATTCAGGGGTAAAACGTGG
TGTGCCCATAGACTCCTCCTATGCTCATATAGGGTAGATTTGTCTATGGGCCCTCCATAGCCTGATACAG
GCTGTCCAGCTTTATGGGGTCAGTTCATTGGTAGGAGTATTTAATTCTTCAAACTCTTTTTTGGCTTCGCT
AAGGCTTTTGGTAAATTCAGGTGTATTATGAAGTACTGCAACAACCGATGCTCCCATTAAACGCCCAGC
CTCTACATCGCTTTGCCAATGCGCACCGCAGATGACCCGACTTTCTCCAAACTCATATCCACGTCGAAGT
ATTTCCGCTTTACGTTGAGGATTAATCTCCGCAAGTATCAGTGCTACTGCCCAACCAAAGGATGCATGA
CCAGAGGGATAAGAGCCAGTGATAGCCATGTTCTCATCTTTATCAGGTGTACAGGTTGCGTCTTTATAA
ATAACGAATGGACGAACTCTTTTATAATATTCTTTGGCGTTACGCACGGCGTAATCATGGCTATCCTGTA
GTACTTGTGTTAACAACTGATATAAGATTGGGGTATTTTCCCTTGAAATATCCATGCCATAAGCATCTGA
AAATGCAAGACCAAAATTCTCGTAGTATGCATCTTCGCTAGCGAGACGTACACGATTAGCATCCCGGAG
CGAGCGGCCGAATTCATAATGAGCTTTGTCAGCCTGAAATACTACTGAATCCTCTGCCGGAGGCGGCG
GAAGTATTGACAAACTGTCTGGTGAAGTTTGTTGAGTGAGAAAACCTTCTGCCTTCAGAGCATTTGCTG
AGGGGATAAAAATCATATTTGTAGCAATACAAAAAAGAAGAAAGTTTTTGGTTTTCATAATTTATTTCCT
TAGCATAATTAATTGATGATTTTGGTCAGCTGTAAAAAACGAGCTGTGTTACAAAAAACACAAGTCATG
CGGAAAAATTTGAACAGAGTGATGGAAAAACAAAAAATAGTCTTATATTAAAGTAATAACATTATTTTT
CATATTCACGAAAAATAATGTGGTAAAAGAATGATGTTTTTTATCTATTTGTTCGTATCGTTTTGTCTCTG
TATTATGATGATTTGTGATTTTCCCTCATAGGGAAGCACTTTTCTACTAAAGATAAAATAACTTCATCTTA
AATGCAAACAATTAACTATAGGTTAAAAATITTATACTAATCCAGTTCTTTATTAATAATAAAAGTCTTTT
TCTGAGAGCTTCTTTTTACTGTATGCTCCGGATCAGCAGGAATTGAAGTTGAACGATAATGTGAGTAGG
GAAAAAGCTCTTGACCATAGTGAACTCCATATCCATGATAGCCGGATATTTTTAGACCATCACTCAATGA
CTCATCGTTTTCAAGGTGTTTTTTTATCTGCTCTAGCAAAGATTCTTTTTCCTTAAAAAAAGGCAGAGAGT
TAAGGATACAAGAAAGACTTTCAGCAGGGGCATTGTTAAAATTTTTAGGAGCCACTTTATCAGCGGAG
CCGCATGAAGTAAAACGGATATCTTTCACATTTAGAATACCACAGTCTTTCATTCTTGAAATGATTGTTT
GTACATCAATAAGTTCCGAACCAAGGCCTAATTGATGAGAACCAGGACTACCGTGCCCAAGAATATAA
ACCTTATTATACTCTTTAAAATCAGTATCGTTTACTGTTTTTATTTTCGCTTTTTTTACAGAAAAATAAGCA
GCAGCATTCAATGCGTCATCATCATTATTTATTACTATTGGTTCACTCTGTGGTAGCCATGATTCAGCCAT
TTTTAGAAAATCATTTTTACTTTTTTCATTTTTAAAAACATAATCATATAACATCTTGTTCTTAAATACTCTT
GGTATTTGTTGTCCCATAATAGATAGAACATCATTGCTATCACAAAAACCTTTCTCTCCTAAAAAAACCA
ATGCTGCTTTTGGGTGAGTACATGATGAATTTTTTACCCTTTTTATTATATGTGCAAATGCAATATCTGAT
ATGCTTTCATTCTTTTTATTGACTATTCTACAGTATGGTCTAACACCATCTAAATTCATACCTACATGACTC
CATATATTGTTTTATAAACATCATAGTAAAGGGTGTTATATTTTCATATAGTATGCAAGCTTCTTTGTATA
CTATGATTTAAAAAAACGGAACAAAAATGCAATTATATTCTGGCATTGTTTAATGCTGTTTGTCTTTTTTA
TTCATTATTTCAATGAAAGAATTTCAACTACAAAGTTAATTGATCCTACCTACATAATATAGACACAGTA
CCTAAAGTACCGTGGAGATCATCCGGGGAAGCCCCGGGTCTGATACCAGTGCAGGTAGACCGCTATCT
TGTTCCGGTAATGTTCTGCCGTTCTTTACGGCATCACCTCTAGCAGAAACATCGCGTAACTGCGCCAAGT
ATGATGAACTTCTCTTACCGCTGCGATCTGGTTCAACAATATGCAGATGGTACACAACCTCTTTAACCAG
CCCATCCGGATTATTCAGAGCCGCTTCCGCAATCCGGAATAAAAGTGCTGTTTTACCGTGAACCCTCTGA
AAATCAGCGAGCAGTGTTTTAACCAGCCGACGTTCAGAGCGTACTGACAGACGGTGGACAATTTGAAT
AAACAGTTCAATCAGACCGTAGATGCTCTCCCGTTRGMRACMTRCTGGRARCRRTRGAAMAMMGCTT
CRGCAAMGAGCTTCCGGCGTCTCCAGTGAAGTGACTGACGGATAATGGTTCATGCTACCGGGCTAATG
AAACACGCCAGTTCACCCGGATGTTGGGACTTTGTTGAACCGAMGMACACGGCGGTGCGGAGTCCGG
AGAGTAACAGAATAGCAAAGAGCTTCGTGAAAACAATAAAGTGTGACTACATCAGTATCATGCCCAAA
CCTGACGGGTTAACGGCAGCAAAGAACCTTGCAGAGGCATTCGAGCATTATAACGAATAGCATCCGCA
TAGTGCGCTGGATTATCGCTCGCCACGGGAATATCTGCGGCAGCGGGCCAATGATAACAGGTGTCTGG
AAATATAGGGGCAAAGCCACCCACGAACGATCCAGCATATAATCAAGAAATTAGATTTCATTTAACATA
AGATAGATTATGTGCACCAAGAGGCGTTTCCTTAATTTCTGAATTTACAGGCTCTTTTGCTCTCTTTGCTA
TGCTGTGGATTTGGAAAGATAGAGTGCTGGCTGATTATTTAGGTATTTTCATCAGCAT
PINV_53G sequence
(SEQ ID NO: 34)
GTCCCTGATAGCACTGTTCCATCAGCGCTTTCAGGCGATAATAGTAGACCTTGGGTAGCCTCTGCCCAT
GTGTAACTTGATACCCCGAGAACAGCCACGATGATCTAAGCGGTTCTTATTTCTCCGGTCTCAGGGTCG
GTAATGCCAACAGTAAGTCCACAGTAATCAACGAAGAGTTTTTTGAACCGCCCCGGAAATCCTGGAGA
CTAAACGCCCTGAGAAAGAGGTAAACAGAATGACTAAAAATACACGTTTTTCCCCCGAAGTCCGTCAAC
GGGCAGTTCGTATGGTTCTGGAAAGTCAGGGCGAATATGACTCACAATGGGCGGCAATTTTTAGCCAT
GAAAACGTAATCAACTCGGTGAACCAATTCAAAAAATATACTTTATACCTGCGGAAGTGAGAAAACCAA
TATATTCAATACTTGCACTATTTTTAATGGTGCCAGATATATTGGTACTTTGCTCATAAAGAGATGTATCA
CCTTTTTTATTAGAAATGTAATTTCTGGAGCCTTCTATATAAAATTTTGCACTGGGGGTGACATAGTATC
CAATATTCGCAGCAACAGAGCAGTAATTTTGGTTTTTTATTTCATCTTTAAAAATTGTCTCAGTCTGATAG
TGTTTATCTGTATCAGATGAACGAACCCAACCACTATATTTCAGTTCTGCTCCAAACTCAAAATTCTCATG
GCGGTAATTAGCAGTTAATCCAATATAAGGTATTTTGAAGAGCTGTTTGTAATCTATTGTTCTTTCACCA
CTAGGATGTGCCCCTTTTTTATTCCTGTTCCCCCCGTTTTCACTATAAATATAACTCCCTCCCATTGCATTA
AAACTGTAACGACTCTCTTGGTAACCTGCTATTAGTCCAAGTCGATAATCCAAATTATTTAATAACCAAC
CTTTAAGATTCAGCTCGAATTCGTTAGCATCACGGAGATGCGTATTGGGATGCCAGCTCTGGTCGGTCC
ATACCTGAGGGGTATTGGAATTGTTCCAGTCTTTATCAACCATGCTTGCTTTCTGGTTACCTAAAGTAGT
CCAACCGGAAACTCCGAAAGACACTTTTGGAATTAGCTTCCAATCGATGCCACCTCTAACAATAGTCGC
ATTACTGTATTTCCAGTCCAGTTGACTAATTTTTCTCCCCCCTTCCTTGGGATGATAAACGCGTTCTTTTG
TTTTGCCACTCAGAGATCCTAGACTAATATCCGTACTGATGTTGTCCGGTATGAAAAGTGGATAGTTAG
TGGTAGCATGTGTAGTAAAGATCGCTGGTACACAAAGTGCAAGTACAAGGAATTTTAATTTCATCGAG
AACTCTACTTTTTTGGTTGAAATGTCCATGATCTTCTTACTTTATAAGATAAACGTCAATGCCAAGCATTA
TTTTAATTAAATATTTGTTTATGTTACCAAGAACATATCTTTTATTATAATATTTATTGAACTTTTGTAAAA
ATAAAAAAAATACCTCATTGTGCGAATAAAGTAACGGGGGCCATAGATTTCTCTACCTACCTTTTGGTG
TTCATACCTGAGGCTGATAGTTTCAGGATTCCGCGACAATTCTACTCACCTTCTTGTTCTCTCGCCAATAT
GAAATCAATCAGAAAAGCGTTCCATAAGAACAACGATAAGACAATAAATTGTCATCACGGCTGCAATA
GCCAACGTGCCTTTCAGTACCAACATGACAACCTCCTGTATTTGACGTAACGTGTGCTGATAAATATGTA
TTTGTATCTCAACTGACTCAATACAACACTTAAGTATAGTTTTTCAGATTTTGTTTTTCCCGTGAAATATT
ATGGAAATGGCAGAAGCAATACATTGAATTCCGATTCGAGTGAATACGGCTAGCGGGCACCTCACTTT
AGCACTGAAGCCATCAACTITTATTTGACTTATTTCAATGGAAAGCTTTCTCACTATCAGTACAGGTAAT
TCCCATCCTTTTAAAGTCGAGAGTCAGACTGCGACTAATACAGTTAAAGTGACCAAACTCAGTTATCCTC
TACACATTTTACCTCATGTTTACTTGGACAATTCATTGGTGCTTTTATAAAAGAATAAATATTCTACTAAT
TTAATGCATCACCTAAAATAGTATTTTTTATAATAGAACTGTATCTATAGTATATTATTAAAGCTTAATTA
TTTATTATGCTTTAATGTGGTTGTCCGATTAAGGACTAATCCTTGAAAACGCCGTATAGGGCGGGAGTT
CACTCTATTGCGATAACGTAACCGTACAATATTGTGCGTTTCCCAAATTGACCACTACTGAACTATTCTG
GGATTTAAATGCAATAAAATGGTTGGTTGAAGGTCGTGGTGATGATGTTAGTCCTTTTGAGAATATTCC
AGTGCCAGATCTTACTTATAATAAAGAGGCTTGGCAGTTTGGATAAGCTATTGTCCTTATTCCACTAATA
CAAACTTTTTGTAAAGTATATTAAATATACTTCATTTCATACTGAAATCCCCGGAAGTATTCAATCAAACA
ATTACACCATAAAAGGAAGAGGGAGCCCCAATTAAGTTATCAAGCATGTTTATGATACCTGCAGGGTAT
AAATTTTCTGGCTGCAAGCAGACACATATAAAACGTCTTTATTGATCCTACCCACATAATATGGACGCAG
TCGTAAGTGAGGCTCTGTGAGCCTACCCACGTAATATGGACACAGGCCTAAGCGAGGTTCTGGTTTTCA
AATTGTTCCGGACTGAGGCCGCCACACCAACGGTGCCGTCGCCAGCGATTGTAATCGCACTCGATATAA
TTAAACACTGTTGCCCGCACTATTTCCCGATAGATAAAGTGTTCTCCGTGGAGACATTCCACTTAGGAAC
AATTATATTGTTCATGGTAATGTTTATATTTGAGTATAGAGATTAAAATGATTAGTCCCATCAAGAATAT
TAAGAACGTATTTCCAATAAATACTGCAAATACTGAATATATAGTGAGAAATATTTATCCTAGAGTTGAA
CATGGTTACTTTAATGAATCACCAAACATATATGACAAGAAGTATATCTCTGGTATAACCAGAAGTATG
GCACAATTAAAAATAGAGGAGTTTATTAATGAGAAATCCAGACGACTTAATTATATGAAGACAATGTAT
TCTCCATGTCCGGAAGATTTTCAGCCTATTTCCAGAGACGAAGCCAGTACACCGGAAGGAAGCTGGTTG
ACAGTTATATCTGGAAAACGTCCTATGGGGCAATTTTCTGTGGATAGTTTGTACCATCCTGACTTACATG
CATTATGTGAGCTCCCAGAGATTAGTTGTAAGATCTTCCCTAAAGAAAATAGTGATTTTTTGTATATAAT
TGTTGTATTCAGAAATGATTCTCCCCAAGGAGAACTACGGGCAAATCGGTTTATAGAACTATATGATAT
AAAAAGAGAGATTATGCAAGTGTTAAGAGATGAGTCGCCAGAGTTAAAGTCTATTAAATCTGAGATTA
TTATTGCTCGTGAAATGGGAGAACTTTTTAGTTATGCATCAGAGGAAATAGATAGCTACATAAAACAAA
TGAATGACAGGCTTTCTCAAATCAAGGCTCGAATGCCTGTTACTTAATGTATATTAATTGTATGAAGTGG
TCAACAAAAACTGGCCACCGAGTTAGAGTTTTTCCAGTATCGATTTTCCGATTCGTTTGGGGGTAACCCA
CCGTTATATTCGTGCGGTCTTAGTGCGCTGTAATATCCAACGATATAGTCCGTTATGGCGTGAGCTGCCT
CGCTGAAGCTTACGTAACCCACCATCGGCATCCATTCGTTCTTCAGACTCCTGAAGAAGCGTTCCATTGG
GCTGTTATCCCAGCAGTTTCCGCGCCGGCTCATACTCTGTCTGATCTGGTATCGCCACAATAACTGCCGG
AACTGCCTGCTCGTATAATGACTGCCCTGATCGCTGTGGAACATCACCCCGCCGGGCTTACCACGGGTT
TCCCATGCCATTTCCAGCGCTTTCATGGTGAGCCTGCTGTCCGGCGAGAACGACATGGCCCAGCCCACT
GGTTTTCTTGCGAACAGGTCGAGAACAACGGCGAGGTACGCCCAGCGCTTACCCGTCCAGATATAGGT
CACATCACCGCACCACACCTGATTTGGCTCGGTCACGGCGAACTGCCTTTCAAGGTAGTTAGGGATAGC
AACATGTTCATGACCACCACGTTTATACCGGTGAGTCGGCTGCTGACAGCTGACCAGCCCCAGCTCTTT
CATGAGCCTGCCAGCAAGCCAGCGTCCCATCTGGTAGCCTCTCCGGGTTGCCATTGTGGCGATGCTTCT
TGCTCCGGCCGAACCGTGGCTGATGCCATGTAGCTCAAGTACCTGACTGCGTAATACAGCCCGTCTGCC
GTCTGGTTTTTCAGGACGGTTTTTCCAGTATCTGTAGCTGCTGCGATGAACCCCCGAGTAGGCAGCCTG
ACGGCTGCGGCTTGTCATGGTCTGCAATTACCGTTATAAAAACAGGCAATGTCATTGTCTTTCAGGTGG
TTATATGGCCCGTTCAGCTAAACCCCGTAAACGTAAACCCGCCTCTCAAAGAAGCAAACTTCCCCGCTAT
GTCGTGAAGCTTCACGAAGATGATTTTTTTGACGAAGAAGACGCAGAAGTTCTGCGCTTTGATAGTTTT
GACGATGCCGTTGAATGCTGCGCAGACCTGAATATTCCGTTCTTTGTGGATGCGGGAAACAAAAAGCT
GGTCTTCTGGTTTGTACGTGTTGATGACGAAGGGTATCCGGAAATCGCCCGCTGCACGGAGCGGGAAT
TTGCGACCATTCTTTCCGGTATCAGCGCCGGCGGTATGTACTGCCCGGAGTGTGGCACGGTTCACTGGC
CGGACGGAGTCGCCCCGCCCTTCTGATGCTTCCCCGTTTTGCCGATATCTTTCAGCAGGGAAACCGCTG
GCTTAACTGGCTGGAGAAGCAGCCGGAAGGGGCAGTACGTCCGGTGGTGATTGAGTCGGTGACCAAA
ATCATGGCGTGCGGGACCACACTGATGGGGTATACACAGTGGTGCTGTTCATCACCGGACTGTTGCCA
TACAAAAAAGGTCTGCTTCCGGTGTAAAAGCCGCTCCTGCCCGCACTGCGGGGTGAAGGCTGGCGCAC
AGTGGATACAGTATCTGCTGAGCCTGGTCCCCGACTGCCCGTGGCAGCATATTGTGTTCACACTTCCCT
GCCAGTACTGGTCCCTGGTGTTCCACAACCGGTGGTTACTGACAGAGATGAGCCGCATTGCAGCGGAT
GTGATACTGGAAATCTGCCATCAGGCAGATGTGGAGCCGGGGATATTCACGGTGATCCACACATGGGG
GCGTGACCAGCAGTGGCATCCGCATATCCATTTATCGACAACTGCCGGTGGTGTGACGTCGGGCCACA
CCTGGAAAAATCTTCATTTTTACGCCCGTAAGGTGATGAGCATGTGGCGCTACCGGATAACACGGTTGC
TGTCACGGAAATACCCTGACCTGGTGATGCCGGATGCGCTGGCAGCAGAAGGAAGCAGTAAACGGGA
ATGGAATCGCTTCCTGGACACGCATTACCGCCGTGGCTGGAATGTCAACGTATCCCGGGTGATGGATA
ATGCCACCCATGTGGCGGTGTACTTTGGCTCTTACCTGAAAAAGCCACCAGTGCCGATGAGTCGTCTGG
AGCATTATGCCGGTCAGGATGAAATCGGTCTGCGTTACAACAGTCACCGAACAAAACGGGAAGAATAC
CTGTTGATGAGTGGAGATGAGTTCATGGAAAGGTTCTCCTGGCATGTGGCGGATAAGGGGTTCCGTAT
GGTGAGGTACTACGGTTTTTTGAGTCCGGCGAAACGGCGGTTACTGGAAGAAGTGGTGTACATCATAA
CGGAGACAGTGAGAAAAACGGCGATGCAAATCACCTGGAGAGGGATGTATCAGAGGTTACTGAAGGT
TGACCCGCTGAAGTGCGTGCTGTGCGGGAGTCAGATGCGTTTTACGGGGCTGAAGCGGGGCTACCGT
CTGGCAGAGCAGGTCCTGATGCATGAGCTGCTGGCCCGGATGCGGTGGTGCGGCTGAGAGCCGCAGA
GGGGAAGTTGCGTCCATTTTGAGGGAAACGGAGCAAAAAACCGCCATTCATACCCTGAATCAATCAGA
GCCATCCAGTTTAATCGTCGGTTCCGTTCATATGGAGCAAAAGTGGTGTTGAAGAAACATCCGTTTTGT
GGTGTTTTTTTAATCTTTTTGGGGTTTTAATTCCTATCGATGAACGCAACCTGACAAGAGAGAGAACTCA
GGTCGGGCTTCAAGCAGCACGAGCCAGAGGACGTAAGGGCGGCAGGCCGAAAACACTGAGTAAGGA
TAAACAGGCCCTTGCTGTTCAACTCTATAACGAGAAAAAACATACAGTTGCGCAGATTTGTGTGTTGAT
GGGGATATCGAGGCCAACGCTGTACAAATACATAGAGTCAGCCAGGTTGTTCAAGAAATGAACTTAGC
GTACGTTTTCGTACGGTTGGGCTTCAAATCCCTAATACGTTGACGGGGCAATATGCAGTTCGCTGCATA
GCGGTCCGACCCCGTACTGCTCACGCAGCTTATCCAGCAGTGGCATCATTTTTTCCAGAGGCGGTCGAA
CTCCGCCTTCGCAAAATAAGCGGAAGCTTGGCGAAGGATATCGTTACTGCGGCGCAGTTCACGATTTTC
ACGCTCCAGCTCTTTCAGACGCTGACGTTCAGCGGTGGTGAGCCCTCCATCACCGCCCCCGGTATCCCG
CTCATGCTGGCGAACCCGGACACGCAGAGTCTCCGGCGTACAGCCAATCTTTGGAGCAATGGAACAAA
TTGTCGCCCATTGTGAGTCATATTCGCCCTGACTTTCCAGAACCATACGGACTGCCCGTTGACGGACTTC
GGGGGAAAAACGAGTATTTTTAGTCATCCTGTTTACCTCTTTCTCAGGAAGTTTAGTCTCCAGGATTCCC
GGGGCGGTTCATTTGAGGATCGCCATGATTTGGCTGTCGGTAAATTTTGATGTTTTCATAAAGAATCTC
CTGCGTTTATGGTACGAGAAAATTCTACTTTTGACGCTGGTTAATTTACGGGGTGATTACCAGACCAATC
GCCAGTGGCGCAAAGCCCGCAGGAGCGCGTTTTTCTGTCGCTCCGGACAAAAAACGCACTGAGGACAA
ACTCGCTCAGCATACAGGCGCTTAACGCATAGCCGCCAGGTGAATGGAGGCCATAACCATTAGCCGCA
AAACCGCTGGTTGTCGCATCAAACCCAGCCTTACCGCTGGCGATACCATACAGCACTGCCGCAGCGGCA
ATACCGCCGATAACCTGAGCGATAATGTAAGGTAACACGCGCGCTGCAGGGAAGCGTCCACCGGCCCA
CAGACCCAAGGTCACCGCGGGGTTAAAATGCGCACCAGAGATGTGCCCGACCGCATAAGCCATGGTG
ACTACTGTCAGACCAAACGCCAACGCCACGCCCAAAAAGCCTATACCTAACTGTGGGAAAGCAGCAGA
GATCAAGGCGCTACCACAGCCACCCAGAACCAGCCAGAAAGTGCCGAAAAATTCGGCGCTAAAAGGTT
TAAACATACACTGATGTCCTGTTTGTTAAAGAGCAGGCATCAGGGTAAAGGCGGTCACGATGTACCGC
CTAGCGGGGATTTGTAAGAGAGGTGAAGAATTGGTGTGGAAGGTTAAACCTAAGCAATCACAGGCGA
ATGACATTGCTCGCTTGTGATAACAGCGATTTTTTCTGCTGCCAAACTAGATCGCAAATATCATCAGTTG
GATTGAATGCCGCTGGTGCTTGTAACAACAAACTGCGAATTCGCTCATGATCAAAGTCATGACAAGCTT
GATCCAGCTCTTTAAGTAAGAGCGATAGATCCTGCCACGGTAGCATCACTTCGTTGGCCGTCATAATTC
GTGGATGAGAGGTACCTTGAACTGAATCACCAATCAGTAATTCTTCATACAGTTTCTCACCTGGACGTA
ATCCCGTAACTTCAATGGCAATATCGCCATCTGGATTTTTATCATCCCGTACACTCAAGCCACTTAACCG
GATCATGCGTTTGGCTAAGTCATAAATCTTGACCGGATCGCCCATGTCTAAGACAAAGACATCGCCGCC
ATGCCCCATCGCCCCCGCTTGAATCACCAACTGTGATGCTTCCGGAATTGTCATGAAATAGCGAATAAT
GTCACGATGAGTCAAGGTAACTGGCCCACCTTGGGCAATCTGTTTTTCAAACAACGGCACGACAGAGC
CCGAAGAACCGAGTACATTACCAAATCGCACCATACAAAAGCGAGTTTGGCTTTGCCGTGCAGACAAA
GCCTGCAATACCAATTCGGCCAGACGCTTAGTTGTCCCCATAGTGTTGGTCGGGCGCACCGCTTTATCG
GTGGAAATCAACACAAAAGTTTCAACGCCACTTTCGATCGCTGACTCAGCGCAGTACAAGGTACCAAAC
ACGTTATTACGTACCCCTTCCACCACATTATGCTCAACCAGAGGCACATGTTTATAAGCGGCCGCATGAT
AAACCGTTTGAATACCAAAGGACTTCATCACCATCTGTAAGCGATTCTGACGCTGCACCGAGCCCAACA
GAGGGATCACTGGAACATTCAAAACTTCTTTGTCACACAGCGCCGAGAGCTCTTTCTCAATAGCGTAGA
GGGCATATTCAGACAGTTCAAATAGAACCAATTTGGCCGGCTTATATCGAACAATTTGACGACAAAGCT
CAGAGCCGATCGAGCCTCCCGCCCCAGTGACCATAACGGCTTTGCCAGTAATGTTTTCGGCCATCAATT
TGGCATCAGGAGCAACCGGATCACGACCTAGTAAGTCATCAATCGATACTTTTTTTAGATTACTGATTTG
TGCTCGACCTTCGACTAAATCGACCATACCCGGAATAGAGAGAACTTCACACGGTAGATGCTCTAAACG
AGTAATCACTTTCTGCCTTTGTGACTTACTGACGCTCGGCATCGCCAAAAGAATTTTCTTTATACCATAGC
GATCTACAAGGTAACTCAGTTTATCCGAGGGATAAACTGTTACACCATGAATGACAGCCTTATGCAAGC
GAGGATTATCATCCACAAATGCCACAGGAAAATATTCTTTTGCCTGCATTAATGCTGGCAATAATTGTCG
GCCAGATTCACCAGCGCCATAAATCAAAACAGGCGCACTACCCTTAACTCCATAGTTAAGTATCATTCTA
AAAAGCAATCTAGAGCCTGATGTCAGTAAAAGTAAAACCAAAAAATAAATTCCGACAACAGTGCGCGG
CAACATTATGGATAGCGAATATGATATAACGACAAGAGATAACGTTGAGGCCAGAATACCAACTGGTA
TTAGCAACATTATCTTTGCACTAACATACCGTAAGACTGCCCGATAAAGCCCAATCCTGATAAATATAAA
CACGGTAAATATGGTAAGAATAGCTGCAGCAAACCACATCGGTGCACTAAGAAAAGCCGTTTGCTCAT
CAAGCCTCAACCAATAAGACAACCAAAATGAGAATATCACCATAACTACATCAATGCAGATGATGATAC
CTCTCTTAACAATTCTTGGCAGCTCCAATATTCTATCAATCATCCTACACTACCTTATTATCGCGACAAAA
TCTTAATAATAGTTTTCCAAATTATCACACAATCATACTTTACTGAATTGTTAGCAACATAATCTAAATAA
TATCTTTGCTTGATTGGAAGAATTATATCTATATAAGCCCTACGTGGGTCATCATAACTAGACAAAATCT
CATTTTCATCTACCATTTCTATAGATGCTAAGTCAGTTATCCCTGGCCTAACCGATAAAACTTTTTCCCTT
ATATCATCAGGATACTCATCAATAAACTCCCTCACTTCCGGTCTTGGGCCAACCAAACTCATTGTTCCAG
AAAGAACATCTATCAATTGAGGAAGCTCATCGATTTTGTACTTCCGTAAATACCATCCAACTCTGGTTAC
CCGAGCATCTTGACCAACTGTTATCCGTCCTTTTTTTTCTGAATCGATAAACATCGTTCTAAATTTATGTA
TCCTAAAATCTTTCCCAAATCTCCCAACTCTATACTGCCTAAAAAAAACCCCCCCTTTAGAATCAGCAACA
ATTAACATTGACACAATGATAAAAACAGGAAATAGAAAAAGCAGGCCTAAGCCTGCCACGATAACATC
AAAAATACGTTTCATTTAATATAAAATACCATTACATAAATAATTGTCTAATCGATTTGATAACGAACAA
CTGATCTTGATCCGTCATTTTAGTATAAAGAGGAATAGATATTTCATTTAAATAACACTCCTCAGAAACT
GGAAAGTCACTGGCGTTCAAATTATATGTATCACGCCAAACCGGTTGCTTATGCAACGGTATAAAATGG
ACAGAACAACCAATTCCAAGATCTGATAACTTCTTAATAAAATCATCGCGATTAATAGCCGAATCAGTTT
TTAAGCGGATAGGATATAGATGCCAAGCATGAATATCACTAGCATTAGTAGGCCATTCAGGCAATTCAA
GTGGCAATTCTTTTAACGCATCATCGTAAATTTTTGCCATTCGTTGACGTTTTTTCTGAAAATCATCGATC
TTTCTAAGTTGATGAATACCGATTGCCGCACAGATATCAGGCATATTGTATTTAAACCCTGGAGCTACAA
CCTCATAAAACCAAGAAGGAGTTTTAGACTGGTACCGGTCAAAAGCGTCACGACTGATTCCATGTAAAC
GCATTACCTTACAACGCTCAATTATATCTTTATTTTTTGAAACAACCATTCCGCCTTCACCGGTTGTCATA
GTTTTATTGGCGTAGAAGCTAAAAACCGTAGCATCTGAATCAAGCGTTCCTATCTTACTTCCTTTATATG
TTGTAGGAAATGCATGAGCGGCATCCTCGACAACCTTTAGGTCATATTTTTTAGCAATTGATAAGATTG
AATCCATGTCACAAGCTAATCCAGCGAAGTGTACTGGAATAATCGCCTTTGTTTCATTAGTAATAGCCTT
CTCAATGGCATCAACTGATATATTAAATGTTTTACGATCTACATCAACAATTACAGGATCAGCACCAAGG
TACCTGACAATTTCGGCAGTAGCAGTGAATGTATATGATGGGACAATAACTTGGTCTCCCGGCTTTACG
CCAACAGCTTCAAGAGCCAAATGTAAGCCCGACGTAGCAGAGTTAACAGCTAATGATTGAACGTTCGC
TCCTAGGTAATTAGAAAATTCTTGTTCAAATTGCTTAGCCTTAGGACCTGTCGTAATCCAACCTGAACGT
AAAGAGTCAATTACCTCTGCAATTTCTTCTTCGCCAATTTCAGGTAACGCAAAAGGAATAAAATTTTTCA
TTTAAAATACCTTTTCAAAAAAGATAATAACGATCCACATCCATAAGAAATATGATAAGACAAAAATAT
AAAAGGAAGAAATACTCTACCTATAAAACTTCGCCCATCTCGAATTGAAAACAAAATAGAAATAAGAAA
ATAAAAAAATAAAAAAGATAAATATATATAATCACCAGTAGCAAAGAAAAGAGAAAAAGATACAATTA
AATACAAAACAAAAAAAAGTGGCACTATATGTCTAAAATAATATGCTCCAGACATAACAACCCAAAAAC
CATCAAGAAAACCTTTCTTTATGAAATCTTTAAAATTATCTCTAACATAATACTCAACAAATAAACTATTA
TCTGTATATAATGACAAATTATTGCTTAAAACTCTCTTATGAAAATCAATATCTTGATTTCTATCTAATAC
TTCATTAAAAAGTCCAACATCAAAAAACACATCTTTATTATATAAAGCAAAGACAGCCGTATCTGTTTTT
TTTAGTCGATTATTTTTGTCTACGCACCTAAATGGAGAATTACCAACACCAAATTTCGATGCATAAAAAT
CCGCTACAATTGAACCAAACTTAGTTTTATAAGAATTAGTTAATACACCTCCAACACCAACAACGCTATC
ATCAAACTGCATAATATTAAAATAATCATCTAATAATTTAGATATATAGTTATTTGGAATATCACTATGT
GCATCAACTCTACACACAAACTTACCATTAGCTTCTAGCACCCCAATATTCCAACCCGTAGCAAGAGTTC
TTTTTTTATTATTGATAATTTTATGCTTAATGTTGCTATGTTTAGAAAAAACATCCTGAACAATAGATATT
GTATTATCAGATGAATCCCCATCTACAATAATCAATTCAAATTTATCTAATCCAACATCTTGATTAATAAA
TGCCAAAAGAGATTTTTCAATATACTTCTCTGCATTAAAGCAAACTATAATTAAAGATACTAGAACATCA
CTCACAAATACCACTCCTCATAAAATATAGTTTTCTTTTCCCTGAGTGAACTGAATCAACAAACAAAAAT
CTATTATCAACCGAGATCCTTGGATGTAAGTCACATCGAGAATAAGAAAAAAAACTCATTGACTCATAA
AATAATCCAAGCTCGCGATAATCATTTTTGACAAGGTCATAAACAAACAACTTTTTAATTCTATTTTTATC
TGGGTAAGTATCAGATATAATATATCTATTATGAACTATAGTAGGATGTCCATCAGAATATTTTCTTGCA
TCAAAAAACAATTTCTCTGTATTACAGGATTCAATTGACAATCTATAATAATTTAGCGAATCTATTTCTGC
ACCAAAAAAACCTATAATTTCATCATTCCCATTCCAACAACAATGACTAATAATCGATTCATTTATTAGGA
CTCGAACATTATCATTTATTAAATTCCAATGTATTAACCTTTCACGCTTTCCTTTAGGTGTATAGTATCGA
TGTATGAACATAACGGAACGTCCATCGGGAGCAAACATAAAATGATTGACCACATGTTCAACATCAATA
TTACATTTAAAATTAGTTCTCTTTAGCATTTCGTCTAAGGATAAAAATAATTCTATTCCCCCCGTGTTCAA
ATTAATGATTGAAATATCTGTAGAAAATGATTTTTTATTATAATAACCATAATCGCTATCATATTTAGCCA
GCCATGAGAAATCTAACGTCACAGCCTTGTAATTATTCACATCATAAATCGGATAGTTTATTTTTTTTATA
ACCTTCATCAAAGACAAAGAATAGACAACACTAATGTATCCATTATTTTCATAGTCATTAAAAATTATAT
TGTCATCATCAATCCACATTAATCGGCTTCCCTGCTGCCAATTAAATGCTCGCGTATCATATAGCTTATAA
ACTTTGTTATTAAGAAGGTCTTTTACGCAAATAGATATATACTTAACTTTCTTTGGATGTCGTTTAGTGCT
AAACGCACTAGAGTGGAACAATACGTACCGCCCATTAGAGCTCATTGGACTATGGTCATAATAGCCAAA
AAATGATTCTTCAGAATCATCAATTGAAATCTCGTATATTTTTGATTGAATAACCGTCTTATTTTTATTCA
AAATAATTCCGAATAATGATGCGATATATAAATAAAAGAACTTAATAACATCCCTTACCTTAGGATACTT
ACTCAAGGCTAAGGAAATTTTTCTTTCAACATATTCTAACAACATATTCTCATCCTAAATAGCTGCGCTTA
ATACCGCTACACTTTTGCCAGCCCATGTTTGCCTCCGGGGAATGGGCTGACGGTTTCCATAAAATGGCG
AACTTTTTTCAACAGTTGCCACATTGAGCTGCACTGATGATTACGCGTTATTGTGTCGTGAAGTGCCTGC
CATAGCCGTTCAACATGATTCATCCATGGCGAGTAAACCGGCTGATAAATGACCCTGAACTTCGGGTTC
TCCTTCAGCCAGCTCTGTGTTTCCCGGCTTTTGTGGATAATGTAGTTGTCCACGATCAGCGTGATGGTTT
TCGCCCGACGGTATGTCGCTTTAAGCCGCTTCAGCAGGCTGATGAACAGCGCCGAACTTTTGCTGTTGC
CGCCCACACAGCTGACTTTACCTGTCCCGCTGTGCAGCGCTCCGGCCAGATAATATTTTTCATTCTGTCC
CGGCGTGACCACCCGTTTTTGCTGTCCGCGCAGTTGCCAGTCCGCACCGATTTTGGGATTAAGATGGAT
ATCCACTTCATCTTCATAAAAGACCGGATGCTCTGCGCTGCATTCGTCCAGTGCTTTATGGATTGCTGCC
ATCTTTTCATCTTTATGCGGGTCACGGATACGCAGAGTTGGCGCAGCCCTTCGCCACACAATCCCCGCA
GACGGCAACCAGCGGCGAACGGTTCCGGCATTTAACTGGCAACCGGTTATCTCATTGATTTTTATTGCC
AGCAGTTCTGTACTCCAGCGTGAACGCTGGTAGCCAAAGTCGCCGGGAGAATGTTTTACCAGCTCACGT
AACAGTGTGCAGATATGCTCAAACGGCCAGCGACGGGCACGCCCGGCAGGTAATGATTTCAGTCCCTC
AACACCCGACTGCGTGAACCAGTTAATCCAGCGTCCAACAGAGGAACGGGCGCAGCAGAGCGTTCTG
GCAACGTCGCTGACACGGTCGCCCCGGTGCAGCATCAGCATGGCAGTCAGTCTGCGGGCATAATTTTT
ATCGTGTGTTTTATGGATGGCTTTCTGCATCAGGCGTCGTTCGTCACGGGAAATTGGTGCTATGATCGG
CATTGCTCAGTCCGGTTGGTGATTTGTTTTGATTTGGCGATTGATCAGATCGCACAATCCGGGCTAAGTT
CCCTCAAAGTGATCTACTATTCCGCGCAGCTATTTAGAATTACGTATAAAAACCACCGATAAAAAAATA
GAAATATAAATATATATCGCTGGCGTCAATATATACTCATTAAAAAAGACATAGAAAGCCAAAGCAAAA
ACGCATCCTTTACCAATATACCCTCCGCATTTAAAAATTGGAGACAGCATTATATAAAATACAGAAATAA
GCAAAATAAAACCAATAATCCCTAACTGAGCCGAATAAGATAAATAAAAATTATGCGCCAACCCAAAGT
TGGTACTTCCTAAAAACTTTTTATCTATAGCCTTTACATAATTTGCAACACCTAATCCAGTGCCAATTAAA
TAATTTTCTTTTAAAACATCAACCCCAATAAATACTAACTCAACTCGACGATAATCATTCTGACTTTTTGT
AAGAGTGTTACCACCAGAGAATATAACCTGTGACGAAGACAGCATTCGTTCAAGACGAGTGCCTATAT
CAGTAAAAGAAATACCTAAAGTTAGAAGAGGAACAAAAAACAATGATATTAATAACTTTTCCTTCTTTG
TTAACTGAAGAAAAACAAAAAACAACGATATAGCAAATGATATTATTCCTGCTCGGCTATCCAGAAAAA
GAAAGAACAGAGAGTACAATACAGATGAGACATACCAAGACTTTCGACCACGATAAAAGTATGAATAT
ACACAATAAAAGCACACTACTATAATTGCGGTGGCATTTCTATTTAAATAAACTATAAAATTACTATCTT
CAGAATAACCAATAAATAATTTTACAACTCCACAGATAATACCAAAACACATTACAACATTTATATTTCT
AACCGAATGAGTTAAATAAAGCTCATCTTTACCAATGCAAAATGACACCAAAAGAACCAAAGCTATAAA
ATTAGAAAAAAACGATAATAAAATTACGACACCTGATATATCCGCTGATAACAACAACATCTGAATGCT
GATATAAATCAGGAATATGATGCCTAAAAATAATAATTTCTTTGTCCACAGAGAAACGTGAAACCTCAA
CGTTATTATAGATGTTAAAACAACAAAGAAATAAAATATTAGATCTGGAATATAGCCAATAGGAACTCC
AAAAAAACGCTCTAAATCAGAGTTCATTTGTCCATAAAAAAAAACCGGAAGGATATTAAATAACAAAA
GTACAGGATATAGGTATATTAACATACTTAACCTTTATTAGGCTTCCAATCGAGTCTAAACATATTAATG
GAACGATATAAAACCACAACATATTAATATAACAACCGCTGCTGACCATTAATAATAAAATAAAAGAAA
ATTGGGACAAAGATCATCATTAGTGAATACTTATCATGAAGAATTTTCCCACAATCTTTTTTCACAGAAA
TAATATAAAATATTCCAATTACTAATGTTGCAATGGCACTTCCAAAAACCGCCCCACTCCATCCATACCG
AGGTATAAAATAAAAGAAAACCGCCATTGAAATGACCACACCAATACAATACGATTTAACTCGCTCGCT
TTGATTTCCACTACACACAAGATAAAGTTCATTTATTAGCCAAAAATTATATTTAATAAGAGCCATAAAC
ATTATCAAAAATGTTGCAGAAGATATTACCTTATACTCATCACCAAACAATATCCCTACGAAATAGATGC
CAAAAAAATAATAGCATAAACATAGTAATAAGACTAACAGTGATATATATCGTTGTAATTTCCTTATATT
ACCCCATTCAGAATTTTTCAGATAATAAGAAAATGAAGGCAATAAAACAGTATAAATAACATTAAACAC
TATTATTAGACCGGTATTAACCCTAGTTACCACCTGATATATAGCAACACTATTATAAGAGAAATATTCT
TTTAATATAAAAAGGTCAATACTAGATAAAACAACATTTAGTAACACCAGAATAGCGAACGGAGTTGCA
TCCAAAAAATCTTTATAAACTAAAATACTTGGTCTTTTTATATGAAAACATGGCACATTGCATTTATGTG
ATGAGAAAACCAAAAGCGAAATTGAGTATATGAATATTGATATCGCACTATAATATAGAGGTTCGATAA
ACAGAGATAAAAATAAATTACACCCAATATATATAATTGTTGAGTAGATATTTATATATAATTCACATCT
AAACCGACCAACAGCCTGTAGCAAGCTAAATCTACCACTTGAAATACAAACAAAATATGCAGATGAAAT
ATAAACAAGCAGAGTTAATGAAATTGAATCAGAAACATAAGAATAAAACATGAAGATTATAAAAATGA
AGAAAAAAACATATGTTTTAACTCCATCACTTCTTGATAATAATTCTGATGCAGAATAACTATTATCAGA
AATATCTTTCACTAATTTTGTTCTAAATCCATAATCAGAGAAGTTAAGCACAAACCAATAAACAGTAAAC
ACATAACTAATAATACCAAATGTAGTAATATCTGACACATGTGTGAAATAAACAAAAACTCCTATTTGAA
ATATTGCTTTAAGTAAAAATGTACCACCTGCATCAATCATTTTTCTAAACATACACTGACCATTTAGAATA
AATACATGGATTTTCACCCCTGCATTAATAACAGAGGTGAAATAGATTATTTATTCATGTTAATATAATT
TAGCCTTTTAAAAATCTCACATACCACGGCATTGAAAGTCGTAATCCCTCTCTGATTTTTATATTTGGTCT
ATACTTTAGTAAATCTATAGCCTTAGTAACATCAGCCTGAGAATGCCTAACATCTCCAGATCTAAACTCT
CTATACTTAATGCTCAATTTATCGATATGGTGAATTAAATTAAGCTCATCATAAATGTAACCAGATAATT
CATTTAACGTTGTTCTATCACCAACTGCAACATTATATATATTATCTTTAGCACTGTCCTTCGCTAATGCA
GATAATATATTCATTTGTATAACATTATCTATATAACAAAAATCACGACTCGTTTCACCATCGCCATTAAT
ATATACGTCATCACCTTTAAGCATTGCTGCTGTCCATTTTGGAATTACTGCAGCATAAGCTCCATTAGGA
TCTTGACGACGACCAAATACATTAAAATAACGTAATCCAATAGTTTTAAAACCATATGTTCGAGCATATA
CCTGAGCATAAATCTCGTTAACATATTTAGTAACTGCATAAGGAGAAAGTGGATTACCAATGTTTTCCTC
TACTTTTGGTAGTGCGGGATGATCTCCATAAGTTGAGCTTGATGCAGCATAAGTAAAACTTTGTACTTGT
GCATTTTTAGCCGCATGTAAGATATTCAAAAATCCAGTAATATTAGTTGCATTGGTTGTTATAGGATCAA
CAATTGAACGAGGTACAGAACCTAGCGCAGCCTGATGTAAGACATGATCAACACCTTTCATAACTTGCT
CACAGGTAGTGAGATCTCGAATATCACCTTCTATAAAGCAAAATCGACTCCACTGTTCAGTGGAAACTA
ATGTITTAACTTCATCAAGATTATATTGATGTCCCGTGGAAAAGTTATCTAACCCAATAACAACCTGGTT
TAATTTAAGTAACTTTTCTAAAAGATTTGATCCTATAAAGCCAGCGACACCAGTAATTAACCAAGTTTTC
GGTGAAAAAATTAACTGCTGTGTAATCTCTTCATAGCGAGACATATAAATATCCATGTATATTAATCAAG
TGCTAAACCTAGCATAAAATGCTGAGGTGTATCATATCAAAGAGGATTTGATTTTTTTAATCACTCTTAC
AAGCGCATATCGATACTTTTTTTATCAAGCACATATTTTAAATCGAACAAAACGTGCTCGTCTTTACCTAA
TGCACGAATAGCGCTCTCTCCCATCTCGCGAAACTCATTGTGTGCAACAGCGATAATTATTGCATCATAA
TGATTAACTTTAGGATCTTCACATAAAGTTAATCCATATTCATGTTGTGCCTCATCGGTAGAACACCATG
GATCTATAATATCTATATTGATATTGTATTCTTTTAACTCTGAAATAATATCAATCACTTTAGTGTTTCGTA
GATCTGGGCAATTCTCTTTAAATGTAAGCCCCATCACTAACACATTCGCCCCTTCAACTTGAATCCGTTGT
TTCAACATTTTTTTGACTAACTGGGAAACGACATACTGCCCCATACTATCATTTAAACGACGTCCGGCTA
AAATCATCTCCGGATGATAGCCGACACTTTGCGCTTTATGTGTAAGATAATAAGGATCTACACCTATACA
GTGGCCACCTACTAAACCGGGCCTAAAAGGTAAAAAATTCCACTTCGTACCTGCAGCCTCAAGAACCTC
TAAGGTATCAATCCCTAACTTATTAAATATAATAGATAACTCATTAATCAATGCAATATTGACATCTCGCT
GCGTGTTTTCAATTACTTTTGCAGCCTCTGCTACTTTTATCGATGATGCTTTATGCGTACCGACAGTAATT
ATTAATTTATATAGCTGATCTACATACTCAGCAACATCCGGTGTAGATCCACTGGTCACCTTAAGGATAT
TAGTTACACGATGCTCTTTATCCCCAGGATTAATACGCTCAGGTGAATAACCGGCAAAAAAATCAATAT
TAAACTTAAGACCTGATACTTTCTCTACAACTGGTATACAATCTTCTTCTGTCGCTCCAGGGTAAACTGTT
GACTCATAAATAATAACATCGCCTTTCTTTATTATCTTACCCAATGTTTCAGATGCTTTAATTAGAGGTGT
TAGATCTGGCTGTTTATGTTTATCAATTGGAGTTGGAACAGTTACAATAAATACATTACACTCTTTTAGT
GCATCTAATGAACAGGCGTACGTCAATTTAGTTGCTTCTAACAACTCTAAATTTGAGCACTCTAATGTAC
TATCGTGACCATTTCGTAATTCATCAATACGAGACTTATTAATATCAAATCCAATCGTCGTTACTTTCTTT
CCAAACTCAACAGCAAGAGGCAATCCAACATAACCAAGGCCTATAATCCCAATTTTCGCATTCAAAGTA
TCGAATTTCATTTTCTTATCCATCATAAAGTGCACATAGAACATCTATTTCGCATTGACACAACAATACGT
AACCCAGTTTAATCTAACTTGGTTTTATCTTTTAATGTCGCATAAACTAATGTACCAACTATAGCAAACAT
TGCCCCTGGTAATGCACCAAGAATCAAAATTAATGCTTTGTTGGGTTTGTCTTTTATAACTGGCTCAGAT
GGCTTCATTTGATATCGATATGCTTGCGCCTGAATGTTATCGCGTAACATTCCTTCTAACTCATCAATTTG
ACGTTTCACTTCGTAATATCTAAATGGGTAAATAATTGGCGCATCTTTAGTTGCTTTTAGCTCTGCTGAT
AACAGTTTTTCGCCGAGCAAGAACTTATAGCTACTTTGCGCATTATCGAAGCCGCCTTTGCTAGAGCGA
TAATCAATGATTCCGGCTTGTTCCGCAGTTGTTAAATCTCGCTGAATATGAGCTATTTGATCAAGCTTTT
CTGATTTTCTCTGGAAATCTATGTCAATAATTTCTTTATTTAGATTAACCAAAAGAGTATTAACATTTTGA
CCAAACTCATCAAGACTTAATGTTACAGCTTGATTATTAACATTCTTTAGATATCCCTGCAGTAGTTTATA
TGCCTCTTGCGCTGAGTCTGCCGATGCTTGTATTATGTAGTAACTTGTATTTCCCTTTTCCTTTGGTGGCG
TAATCACAAGACCTTTATTCGCTTGCTCAGCTAACCAAATTTTCTTATCTTGTTCAGTAGATAAATTATTT
GTTTCCTTTTTATAATACTCAGTACTCTCTAGAAATTTAACTTTGGTGTCTGGCGATTCAGCAATCAAAAT
CAAATCGGAAAACAATCCATTAGCCAACGCATTCGTATCTAACGGTTGATTTAATATTCGGGCATACGC
TTGGTGAAATTTAAGATAGCTGCCTAATTCAGCAATACGTGGTGCTTGAATATAGGTTTTAGCCGTCCA
CTGCTCTTTTGCGGTAAAGGCATACACTGCTGCTAACCCCGTAGTCAGCAACGTGACACATAAAATAAC
CCATTTTTTCTTCCACAAGGTGCCTAAAAGCTCGAAAAGATCGATTTCATCCTCTGCTTTTGGCATATAA
GTTGGATAGACCCCTTGCGGGATCAGATAAGGGGTCTGTTGTGGTTCAGATGCTTTTGACATGAAACCA
ATCCATTATTTATCAATGGGTTAAAAATAAAAATGAAGAATAAGCCTGACAAAAAAGTACGCTACCGCC
CTTGAGTTTCAGCTCCCAAAGCGCTGCGCAGAAACAGGATATAGCCAACTGCGATACAACAAGTGATA
TCTGGGATTAGAGGACATAATCGAGAACACTCCGTGTGAAGAGACATATACAGTCCCACACTTCCCTGC
TGCAAACCGGCGGATAGCCCGCAGAATTTGGAGTGCAGATTATAAAGCAATGCCTGTATAAGACCAAA
TCTGCCTTCAAAATGATAACAGTATGTTTGTAAATTCTGTGAGGCTAAATAGCTGCGCTTAATACCGCTA
CACTTTTGCCAGCCCATGTTTGCCTCCGGGGAATGGGCTGACGGTTTCCATAAAATGGCGAACTTTTTTC
AACAGTTGCCACATTGAGCTGCACTGATGATTACGCGTTATTGTGTCGTGAAGTGCCTGCCATAGCCGT
TCAACATGATTCACCCATGGCGAGTAAACCGGCTGATAAATGACCCTGAACTTCGGGTTCTCCTTCAGC
CAGCTCTGTGTTTCCCGGCTTTTGTGGATAATGTAGTTGTCCACGATCAGCGTGATGGTTTTCGCCCGAC
GGTATGTCGCTTTAAGCCGCTTCAGCAGGCTGATCGAGTAGGCAGCCTGACGGCTGCGGCTTGTCATG
GTCTGCAATTACCGTTATAAAAACAGGCAATGTCATTGTCTTTCAGGTGGTTATATGGCCCGTTCAGCTA
AACCCCGTAAACGTAAACCCGCCTCTCAAAGAAGCAAACTTCCCCGCTATGTCGTGAAGCTTCACGAAG
ATGATTTTTTTGACGAAGAAGACGCAGAAGTTCTGCGCTTTGATAGTTTTGACGATGCCGTTGAATGCT
GCGCAGACCTGAATATTCCGTGAGGTAGCCTGAGTTTAACGGACACTCCTTCCTGAAATAGAATGGCAT
CAGAAGGAGCTAATAATGAGCAGAAAAACCCAACGTTACTCTAAAGAGTTCAAAGCCGAAGCTGTCAG
AACAGTTCCTGAAAATCAACTTTCGATCAGTGAAGGCGCTTCCCGATTATCTCTTCCTGAAGGCACTTTA
GGACAATGGGTTACCGCCGCCAGAAAAGGGCTCGGTACTCCTGGTTCCCGCACGGTGGCTGAACTGGA
ATCTGAAATTCTGCAACTGCGTAAGGCGTTAAATGAAGCTCGCCTTGAGCGAGATATATTAAAAAAAGC
AACAGCGTATTTTGCACAGGAGTCGCTGAAAAATACGCGTTAATCGAACAATGGCGACAACAATTTCCC
ATTGAAGCGATGTGTCAGGTATTTGGTGTATCCAGGAGCGGTTATTACAACTGGGTACAGCATGAACC
CTCAGACAGAAAACAAAGTGATGAGCGGCTAAAACTGGAGATTAAGGTGGCACATATCCGCACTCGCG
AAACATATGGAACCCGGCGGCTCCAGACGGAGCTGGCAGAGAATGGCATCATCGTTGGTCGTGACCG
ACTGGCACGTCTTCGTAAGGAGCTAAGGCTACGCTGTAAGCAGAAACGCAAGTTCAGAGCGACTACGA
ACTCGAACCACAATCTGCCAGTTGCGCCAAATCTGCTGAACCAGACGTTCACTCCTACAGCACCAAATC
AGGTCTGGGTGGCGGACCTGACGTATGTTGCCACACAGGAGGGATGGTTGTACCTCGCTGGCATCAAA
GATGTTTATACGTGCGAAATTGTCGGCTACGCCATGGGAGAGCGCATGACAAAAGAGCTGACAGGTAA
AGCCCTGTTTATGGCGCTCAGGAGCCAGCGCCCACCTGCCGGGCTAATCCACCACTCTGATCGAGGTTC
ACAGTACTGCGCATACGATTACCGGGTCATACAGGAGCAGTCTGGTCTGAAAACATCAATGTCGCGTA
AAGGTAACTGTTACGACAACGCTCCGATGGAAAGCTTCTGGGGAACGCTGAAAAATGAGAGCCTGAG
CCACTATCGTTTTAATAACCGGGATGAAGCCATCTCAGTAATACGGGAATACATTGAGATTTTCTACAAT
CGTCAGCGTCGTCACTCTCGTCTGGGGAATATCTCCCCGGCAGCCTTCAGGGAAAAATATCATCAGATG
GCTGCTTAAAAAAAGAACAAATGGTAGTGTCCGCTATTGCCAGTACACCTCACCGTTCTTTGTGGATGC
GGGAAACAAAAAGCTGGTCTTCTGGTTTGTACGTGTTGATGACGAAGGGTATCCGGAAATCGCCCGCT
GCACGGAGCGGGAATTTGCGACCATTCTTTCCGGTATCAGCGCCGGCGGTATGTACTGCCCGGAGTGT
GGCACGGTTCACTGGCCGGACGGAGTCGCCCCGCCCTTCTGATGCTTCCCCGTTTTGCCGATATCTTTCA
GCAGGGAAACCGCTGGCTTAACTGGCTGGAGAAGCAGCCGGAAGGGGCAGTACGTCCGGTGGTGATT
GAGTCGGTGACCAAAATCATGGCGTGCGGGACCACACTGATGGGGTATACACAGTGGTGCTGTTCATC
ACCGGACTGTTGCCATACAAAAAAGGTCTGCTTCCGGTGTAAAAGCCGCTCCTGCCCGCACTGCGGGG
TGAAGGCTGGCGCACAGTGGATACAGTATCTGCTGAGCCTGGTCCCCGACTGCCCGTGGCAGCATATT
GTGTTCACACTTCCCTGCCAGTACTGGTCCCTGGTGTTCCACAACCGGTGGTTACTGACAGAGATGAGC
CGCATTGCAGCGGATGTGATACTGGAAATCTGCCATCAGGCAGATGTGGAGTCGGGGATATTCACGGT
GATCCACACATGGGGGCGTGACCAGCAGTGGCATCCGCATATCCATTTATCGACAACTGCCGGTGGTG
TGACGTCGGGCCACACCTGGAAAAATCTTCATTTTTACGCCCGTAAGGTGATGAGCATGTGGCGCTACC
GGATAACACGGTTGCTGTCACGGAAATACCCTGACCTGGTGATGCCGGATGCGCTGGCAGCAGAAGG
AAGCAGTAAACGGGAATGGAATCGCTTCCTGGACACGCATTACCGCCGTGGCTGGAATGTCAACGTAT
CCCGGGTGATGGATAATGCCACCCATGTGGCGGTGTACTTTGGCTCTTACCTGAAAAAGCCACCAGTGC
CGATGAGTCGTCTGGAGCATTATGCCGGTCAGGATGAAATCGGTCTGCGTTACAACAGTCACCGAACA
AAACGGGAAGAATACCTGTTGATGAGTGGAGATGAGTTCATGGAAAGGTTCTCCTGGCATGTGGCGG
ATAAGGGGTTCCGTATGGTGAGGTACTACGGTTTTTTGAGTCCGGCGAAACGGCGGTTACTGGAAGAA
GTGGTGTACATCATAACGGAGACAGTGAGAAAAACGGCGATGCAAATCACCTGGAGAGGGATGTATC
AGAGGTTACTGAAGGTTGACCCGCTGAAGTGCGTGCTGTGCGGGAGTCAGATGCGTTTTACGGGGCT
GAAGCGAGGCTACCGTCTGGCAGAGCAGGTCCTGATGCATGAGCTGCTGGCCCGGATGCGGTGGTGC
GGCTGAGAGCCGCAGAGGGGAAGTTGCGTCCATTTTGAGGGAAACGGAGCAAAAAACCGCCATTCAT
ACCCTGAATCAATCAGAGCCATCCAGTTTAATCGTCGGTTCCGTTCATATGGAGCAAAAGTGGTGTTGA
AGAAACATCCGTTTTGTGGTGTTTTTTTAATCTTTTTGGGGTTTTAATTCCTATCGATCAAGCGGCTGGC
ACGTAAAACAATCTGCTTCTCGCGTTCCGTGGAAATCCACGAAAAGTCATCGGCTCCTTCATTGAAAAA
CACATATTCTACTGATTGGAAGCGCCACCCGAAAAAGTATCGTTTTTTTACAGTAAACTTCATTTAGCCG
ACTATAATGTAAAAAAATAAATTTTTTACCAAAATATAACATAAGGAAGTAATATGCTTACACAAACTAT
ATTCCCTTGTCTTCCCAAAAAAATAAGAAAATATCATCCCTGAAGTATCAAATACTATATTATTATCTTCT
ACTGTTACTACAGATGGATATACTGTTTTTAATAAAAAAAACAGCCATATACGAATTGCAAATATCTGCT
ACCAACCGAACGAAAAGGTTGAAATTCACAGCTACAGAAATACAATGGTTGACAAAGATAAATGAAGC
CGGAATTGATAAAAAACAAAGCCAACGGTATTCTGATTTCTGAAGTTAATTTCAGACTCGTTTAAGCTCT
ATTTTCAAAAAAAATATATCAGGGTTTGTTACTAATAACTTCTGAAAACAGAAAAAAAATAGAGCAGTA
ATTCTATTTCAGAGAGGAGTAACTCCATCTATCCTCGATACGTACTGAACGTTATCGTGATATATTCATC
ATGCTTTCTTCACCATTATTCGAGTACAGGGAGAGACGCAATGACCTTAAATACCTTTTTATGGCTGAAT
TACCATTCTCAGGAATAACAGCAGACTCCTTCCCATAGACGGCTTAGGGGGAAAGCAATACCCGGAGA
AAGGGTACAAAGCAAACTGTTTTAAAATTAATAAGCTATATTCTATTTCACTAGAATTCTTATCATTGAT
AAGAAATTTTAGTTATAATGATTAAATCAACCAATATACAGGCAATCGGTTCTGGTATTATGCATCAAAT
AAACAATATATACTCGTTAACTCCATTTTCTTTACCTATGGAACTGACTCCATCTTGTAATGAATTTTATTT
AAAAGCCTGGAGTGAATGGGAAAAGAACGGTACCCCAGGCGAGCAACGCAATATCGCCTTCAATAGG
CTGAAAATATGTTTACAAAATCAAGAGGCAGAATTAAATTTATCTGAGTTAGATTTAAAAACATTACCA
GATTTACCGCCTCAGATAACAACACTGGAAATAAGAAAAAACCTATTAACACATCTCCCTGATTTACCAC
CAATGCTTAAGGTAATACATGCTCAATTTAATCAACTGGAAAGCTTACCTGCCTTACCCGAGACGTTAG
AAGAGCTTAATGCGGGTGATAACAAGATAAAAGAATTACCATTTCTTCCTGAAAATCTAACTCATTTAC
GGGTTCATAATAACCGATTGCATATTCTGCCACTATTGCCACCGGAACTAAAATTACTGGTAGTTTCTGG
AAACAGATTAGACAGCATTCCCCCCTTTCCAGATAAGCTTGAAGGGCTGGCTCTGGCTAATAATTTTATA
GAACAACTACCGGAATTACCTTTTAGTATGAACAGGGCTGTGCTAATGAATAATAATCTGACAACACTT
CCGGAAAGTGTCCTGAGATTAGCTCAGAATGCCTTCGTAAATGTTGCAGGTAATCCATTGTCTGGCCAT
ACCATGCGTACACTACAACAAATAACCACCGGACCAGATTATTCTGGTCCTCGAATATTTTTCTCTATGG
GAAATTCTGCCACAATTTCCGCTCCAGAACACTCCCTGGCTGATGCCGTGACAGCATGGTTCCCGGAAA
ACAAACAATCTGATGTATCACAGATATGGCATGCTTTTGAACATGAAGAGCATGCCAACACCTTTTCCG
CGTTCCTTGACCGCCTTTCCGATACCGTCTCTGCACGCAATACCTCCGGATTCCGTGAACAGGTCGCTGC
ATGGCTGGAAAAACTCAGTGCCTCTGCGGAGCTTCGACAGCAGTCTTTCGCTGTTGCTGCTGATGCCAC
TGAGAGCTGTGAGGACCGTGTCGCGCTCACATGGAACAATCTCCGGAAAACCCTCCTGGTCCATCAGG
CATCAGAAGGCCTTTTCGATAATGATACCGGCGCTCTGCTCTCCCTGGGCAGGGAAATGTTCCGCCTCG
AAATTCTGGAGGACATTGCCCGGGATAAAGTCAGAACTCTCCATTTTGTGGATGAGATAGAAGTCTACC
TGGCCTTCCAGACCATGCTCGCAGAGAAACTTCAGCTCTCCACTGCCGTGAAGGAAATGCGTTTCTATG
GCGTGTCGGGAGTGACAGCAAATGACCTCCGCACTGCCGAAGCCATGGTCAGAAGCCGTGAAGAGAA
TGAATTTACGGACTGGTTCTCCCTCTGGGGACCATGGCATGCTGTACTGAAGCGTACGGAAGCTGACC
GCTGGGCGCAGGCAGAAGAGCAGAAGTATGAGATGCTGGAGAATGAGTACTCTCAGAGGGTGGCTG
ACCGGCTGAAAGCATCAGGTCTGAGCGGTGATGCGGATGCGGAGAGGGAAGCCGGTGCACAGGTGA
TGCGTGAGACTGAACAGCAGATTTACCGTCAGCTGACTGACGAGGTACTGGCCCTGCGATTGTCTGAA
AACGGCTCAAATCATATCGCATAACCCACTGATAATGTTTACTACCGGATGTAGCTTTTTTCATGCCCGA
GATGGTCAGTTAGTTCCGCATTAAGCACCGTTTCGACAGTTAATTTCGTCTGCATACGGGAAAACTGAT
TGAGGTCGGCCTCGGTTTTGAGGCCTTTAGCCAGTTCAGCCGCAAGGGCTTTAAGCTTCTTTTCGTCCAT
AATTTACCTGTCTCCGTTGTTGGAGTGAACATATCAAAAACAGGCAATTACACAATTTAAATTACAGTCT
CAAAACAAAACCAACTGTCAATAATAGTTTACCAGCAAGTTTACTCGCAGAGTGCTAGGCTGTGTCCCT
TAATTACGTAAGCGTTGATAAAAGAGTCGGATGCAGCCCAGTTTCACCATCGCCAGGTAATTTCTCGCC
GTTTTGTCGTAACGCGTGGCGATGCGGCGATATTCTTTCAGCCGCCCAAAGCACCGTTCAACGACATTA
CGATTGCAGTACGCATCACGATGAACCACGGCAGCCTGACGGCTGCAAATTGAAAACCGCCCCTGTTC
GTCCTGTTGTTGCCGCACGCTCCGCAGAATATGTATGCTCTGAGGACCTGTTTTATTAAGGACTCATCCT
CTTATGTTGACCCGCAAAAGTATAGATACCGTTCTGCTCTCTGTTGGTGCTGAGAAGCTCTCACAACGTG
AATGGGACTGGATGAAGATGCTCAAACCCATGGACCCACCACCTGCGATGGTCGCGGCCTCCATCCTG
GAGCGCCGTGGCGACACAGCAGCCCTTACCCGGCTACAGGATACCGGGGGTTAATCCGTCCTCATGTT
GTCCGCTTTTACTCCCCGCCCGCTGAAACGTCTGTTCACGGCCAACCAGTGCTGGACGTCCTTCCTGGAT
GCGGGCGGTCTGCGCGATATCGAGGTTGAGGCTGTCACAAAAATGCTGGCCTGCGGCACGCGGATAC
TGGGAGTGAAGGAATACAACTGCGATAAACCTGAGTGCCCCCACGTCAGATACGTCACTAACTCATGC
GGCAGCCGTGCCTGCCCGTCCTGCGGAAAAAAGGCCACAGACCTGTGGATAGCGACACAGCTGAATC
GTCTTCCTGACTGCGACTGGGTACATCTGGTCTTCACCCTGCCGGACACGCTGTGGCCGGTGTTCGAAA
GCAACCGGTGGCTGCTGAATGACGTGTGCCGTCTGGCGGTGGAGAATCTGCTGTATGCCGCCCGGAAA
CGGGGGCAGGAACCCGGTATCTTCTGCGCCATCCACACGTATGGCCGTCGTCTCAACTGGCATCCGCAT
GTACATGTGTCTGTAACCTGTGGATGTCTGAATAAGCATGGTCAGTGGAAAAAGCTGAGCTTCCTGAA
AGACGCGATGCGTTCGCGGTGGATGTGGAATATGCGGCAACGGCTTCTGAAAGCGTGGTCAGAGGGG
CTGGCGATGCCGGAGTCGTTGTCACATATCACGACGGAATCACAGTGGAGAAGCCTGGTGCTGAAAGC
CGGAGGAAAATACTGGCATGTGTACATGTCGAAAAAAACGGCCGGCGGGAGGAATACGGCGCGCTAC
CTGGGTCGTTATCTGAAGAAGCCGCCGATAGCGGCCTCCCGGCTGGCACATTACAACGGAGGGGCGA
GCCTGAGCTTCCGTTACCTGGACCACAAAACGGGAGAAACGGCGACGGAAACGCTGACACAGCGTGA
GCTGGTCGCGAGGCTGAAACAGCACATCCCGGAGAAGTTTTTTAAGATGGTGAGGTACTTCGGGTTCC
TTGCCAACCGGGTGTGTGGAGAGAAGCTGCCGCAGGTGTACCGTGCTCTGGGGATGGATAAACCGGA
ACCAGTGGCGAAAGTGTGCTATGCACAAATGGTGAAACAGTTCCTGAGTCGTGACCCGTTCGAATGCG
TGCTGTGTGGCGGCCGGATGGTATACCGCCGGGCCATTGCGGGGCTGAATGTGGAGGGGCTGAAGAA
AAACGCGCGGGATATCAGTCTGCTGAGGTATATGCCGGCCTGACACAGGCTAAGTGTGCCCGGGAATC
GGTGCAAGTATAAAAAACAGCCTGTTGCGGTATAAGAATCGACCATGCAGAGAGAGAAAACACCGGA
GTGGTGGGGAAAAACAGAAAGGCTCACGTGGCATAAGGCGCGGACAGGGGTATCGCTTAGTTTTTCA
ATTTCCTATACGTCGAGTCCACAGAGAAAATAACAATTCTGAGTTATCTGCGGGAAAGGTTATTTATAT
GTTACTATCTCATTCTCTCACTGGCGAGATTATAAACGTTACTACGTTCTCGTTTTCCTACCGCAACAACC
GCAATAATTAACATATCGTCAATCACCTGATAAACCAGACGAAATCCCGATGCACGTAGTTTAATTTTGT
AGCAGTCCTTTAGCCCTCTTAGTTTTGCCGACGGTATATGAGGATTTTCGCTGCATTTTTTTAGTTTTTTC
ACAAACTGTTGTTGAATGGTCTTATCCAGTTTCAGCCATTCCTTGAGCGCATCATCCCTGAATTTTACCGT
ATAAGTCATAAATAACTGTCCAGGTCCACATCATGCAGTGGCTGATTGCTGCGTTCAGTAACCAGTTTC
ACCAGTTCCTGGTCATCCAGAGCATCAAGCATCCTTTCATACAGTTCAGCAGGCACACAGTAGAATGCC
GGCTGATTCCGGTTAAGAATGGCGACAGGGTAACCATCTCCGGCACTGACTGTAGCCATAGGATTTTTT
TTCAGTTCACTGACACTTGCACTGGTCTCAGACAGAATGATGTTAGGCATAACGCACCTTTGAGACTGTT
TAACAAGTCTTAACGATATCCCTGAAACGACCACTTAACAAGTCTTTATTACTCACACTTTTGCAAATGC
ATTAAATGCTGCTTGTATTATTCTGATGCCGCGCCAGCGGCATGTAAGGCGGCACGCTGCGGTTACATG
TGATACCGGAGTAAAACCGCCGAAGCCCGGCGTCAGCCGGTACTGATTGACAGATTTCACCTTACCCAT
CCCCAGCCCTGCTAAGCCATACCCGTTTTCAGCCATGACAGAGCTTCTGTGCGCGGTCGGAGTGGTCCC
GACGAGGGTTTACCCGAAGTCGGGGCGTATCTCCGCGTTAGCGGGCCGTCAGGGCCGCTTACGAGCGT
GTACTCGATACGTCCAGCCAGAAGACTGACAGCGATGATGATGTAGTTACAACATTCATAATTAAAAGC
GACTCTGTTCCCGCCCTTCGGGGGGGGCAGCGGCGCTTTTCAGTTATGAGGAGGGGCGTTGTGGTTT
CAGTTCTGCGTCGGACTTGGGTTTTTTAGGGGTTTATGTTTGCATGTTGTAACTAAATCGGCTTCGGTCG
GGGCCCGCCGCTTGCGGTGGGAGGTGCATATCTGTCTGTCCACAGGACAGGCAGTGAACAGGTTTTCT
TTTTAAATGGATGTAATTAAGTAGTTTAAAGGAGATATAAACAGGTGTTTAAAAGATACATTGCACCCT
GTAAGGCTGACGGCTGGCGCTTTATGACATGAACGGTTGTAACCTTATGGGGAAGTCCCTTGCAGTTTA
ATGTGGATAAGCAAAATTCCCCGTCTGTGAGGCGTGTTTTGTATCAAAAACAGGGGGGACCGGATGCA
CCAGAAGGTGGATGATGAGGTTGTTTTTTGTATGTAGCGCTGATTTTTTGTGCACTGGCGGGCTTCAGG
CGTGCGAATGCCTCCGGCGCGTGCCGGATTATTCTGAGGAGGTCACTTTCAGGGGGAAGCTGTGGCCA
GCCGGCTGTAATTACGGTTACGTGACAGAATCATGCGCTCCTTCACACGACGCTCCACTTCACGTTTTAC
CGCCTCACGACTGGCAGTGAAGCGCCCTTCCGAGATTTCACGCGTCAGTTGCCGTTTCACCAGGGTGAC
AATATCCTGACGCTGCCTGTCCGCATCACGACGCGCACGGGCACGCTTCATTCCACGGGACTTAAGCTC
TGTCTGGTAACTGCGGAAACGCTCACGCACAAAACGCCAGGCTTTCGCCATCAGTTCATCCATACCCAG
GGTATCCAGTCCCTGCTTCTTGCGCTGTCTGTTTTCCCATTCCACACGACTGCGGCGCGCGGACGCCACC
GCCTCCTCTGATACATCGAGGGCGGCAAACAGCGCCGGTGTGAACGTGATGTCGGTCGGAATGTTGCA
GCCGATAAGCGGGTCATATTCCGTCTGGTAGGTAATCAGCCCCAGCTCTGCCAGGAACGTCAGGGCAC
GCGTGGCACGGGTGATGGAAAGTGTTCCGGCATCAGACTCCGTCGCCAGTCCGCACTCAATGGCCAGC
GTGGTGATGGAGCACTGAACGCGGTTGGCCAGCGGGTCATAGTGGAAACACAGCCCCTGCAGCAGCG
CATCAATGGCCCGGCGACGTAGTACCGGTGGCATGCGCCGACGCAGACCAAGCGAACGGGCATGTGC
CACATGAATGGCGAAATCAAAACGGGAGGTGAAGCCCACCGCCTTTTCCATCAGTTTCCCGCAGAACG
GAAGCGTCCGGGCACCTTCACGGGGTGTGAACACCGGATTCGGGTTCTTTACCTGGCGGTAATAAGTT
TGTTGAAGATCAGTCACACCATCCTGCACTTACAATGCGCAGAAGGAGCGAGCACAGAAAGAAGTCTT
GAACTTTTCCGGGCATATAACTATACTCCCCGCATAGCTTGATTGTTGGCTATACGGTTTAAGTGGGCCC
CGGTAATCTTTCTGACTCGCCAAAGTTAAAGAAGATTATCGGGGTTTTTGCTTTTCTGGCTCCTGTACAT
CCACATCAGAACCAGTTCCCTGCCACCTTATGGCGTGGCCAGCCATAAAATTCCTTAAACGATCAGTAAT
CTAGCATGCTACGCCACAAAGTAAAGTCTTTACTTTAGTATATCCAGTCTCTGCAGTTCATCTTTGATGAT
TTTCTCAACGAACTGAGCCTGTGTTATCCCCTCTCTCTCGCAGTACTCAACCATGAGATCTTTCAGAGGA
TTTTTGACAAAAACTTTTATCTCTTTGTGTGTAAGACGTTTTCTTGCAACTGCGGCCATTTGTTTCTCAGA
GTCAGTCATAGGCTTACCTCTGCGCACAAACCGCTTTTGACTCAATGAGGAAGTCACTGCATTTTCTGTC
TGCGACATCTCGCCTCCTCAATACTCAAACAGGGATCGTTTCGCAGAGGATACTACAGTTTTTTGAAATC
AGCGACTTGAGAATTGTGGCGAAGATCTGTGTTGGCGCACAAATCAACGAGGATTACTGTCGTTTAAT
GTGATTTAAACTGTGAAATAGTATGGTTTTCAGTTATGGAAACGCCGGGAGCGAGGAAAACTTGCTTTT
TCCCGTTTCCGGGGTTGGACAACTAAGCAACGCGAAGGCGTCAGCTACGATGTTCCGGTGACTGCTGA
TCAGCCCCTGTTGTATTCAGCCATTCTCCGGTCATCTGCAGCAGTTTTCTGGCATCGTGGCTTCTGAGCA
ACGGAATACCAGCCTGCCTCAGAACCTCTTCCAGAAGAATATCCCGGCGTCTGCGTTCCGGTCGTAAAT
GGCTGGCATCATCCAGCTCTACTGCTGCAACAATACTGAACGAACGCAGCTCAACGATAACCACATCAA
CATGCCACTGAGACACCATCCTGAATAACTGCCACCACTGGCGTGATCGTGGCCGGATATTCCCGTTCA
GCTGGACGATATCCGCGACCCGTACCTGCGGACACAGATACCACCGCTTCGGTGATGCTGCCAACTTAC
TGATTTAGTGTATGATGGTGTTTTTGAGGTGCTCCAGTGGCTTCTGTTTCTATCAGCTGTCCCTCCTGTTC
AGCTACTGACGGGGTGGTGCGTAACGGCAAAAGCACTGCCGGACATCAGCGCTATCTCTGCTCTCACT
GCCGTAAAACATGGCAAATGCAGTTCACTTACACCGCTTCTCAACCCGGTACGCACCAGAAAATCATTG
ATATGGCCATGAATGGCGTTGGATGCCGGGCAACTGCACGCATTATGGGCGTTGGCCTCAACACGATT
TTACGTCACTTAAAAAACTCAGGCCGCAGTCGGTAACCTCGCGCATACAGCCGGGCAGTGACGTCATC
GTCTGCGCGGAAATGGACGAACAGTGGGGCTATGTCGGGGCTAAATCGCGCCAGCGCTGGCTGTTTTA
CGCGTATGACAGTCTCCGGAAGACGGTTGTTGCGCACGTATTCGGTGAACGCACTATGGCGACGCTGG
GGCGTCTTATGAGCCTGCTGTCACCCTTTGACGTGGTGATATGGATGACGGATGGCTGGCCGCTGTAT
GAATCCCGCCTGAAGGGAAAGCTGCACGTAATCAGCAAGCGATATACGCAGCGAATTGAGCGGCATA
ACCTGAATCTGAGGCAGCACCTGGCACGGCTGGGACGGAAGTCGCTGTCGTTCTCAAAATCGGTGGAG
CTGCATGACAAAGTCATCGGGCATTATCTGAACATAAAACACTATCAATAAGTTGGAGTCATTACCCAC
CGCTTCATATCCACAATTCTGAACAGTCCTTTCATAAAACTGACTTCCCTTTCTGTCAGCAGCGTCTTCTG
ACGTTGCCAGTATTCACCGCTGGACAGTATTCGCTCTGCATCACCGGTCCGTATACCTGCCGCATGCAAT
TGCATCAGCAAAGGAGAATGCTGATTTACACTTCTGAGGATAATCCGGATAAGTAGCGCTGCAATAATC
AGAACAATGAGAATAAAAATAATCAACTTCATTATTTATCTGCTCCTGGCAATTAATTATTGCTGAATTA
TTACCTTACATATTTCCATATCTCTTTTGGTATCCTGTCATATAACTTTCCGGTGATTAATTCCGCTAACCG
GTGGTCTGCTGCTGAATTAAATGATTCACGTTCACTGTTTGATAAACTGTCTCTGTTTTTATGAATAATTT
TTTCCAGCGTATTGACTGATGAACATCGCCGTAACTGATACAGCCATTCCTGTTTTGTTTTTGCCATTTTT
ATTCCCGCTGAGAACAGATGAATGATAGGATTGCCATCGCTTTATTTACGGTGCCTCCATATCCAGGGG
GGAACCGGATCAGCATCTGACCAGAGCCCACGTTTTTGTTGTCTGGCTTCGTTCTGAAGGACAGGTAGC
ACGGGATCTGTGTTGTACTGCTCATATACCCAGGCAGCCCCGGCCCGGACCATAAACTGATTAATATTC
ATTCCGTCTGGTGCATAAACCTGCCCGAGAATACGACCATAACGATCTCGCTGAAAATATGTGACGGTA
ACCGTTTTTCCTGCGACCAGTGATTTCATCATGTCTGTTGACCACCGGCCATAATCCTGTTTCTTTTCCGG
TGCATCAATATTTACCAGCCTGATCCGCACCGCCTTCAGTGAGTCCATGACCTCGATGGTATCACCGTCC
AGTACACGAACAACCCGTCCATGAATATCTGCAGACAATACCGGCCATGAAAAGATAAATAATACCAGT
GGAATATATTTCCTCATGTTATTCTCCATATCAGATAAATGGCACCTGTATTTCAGACCCCCTGAATAGG
GCAGCACACATTGAATAACACCTTATTTTGTGAAATTAATCACAAAATATAAAAAATCGACTTAATGCG
GAAAAGACTCTGTGCTACTCAGAACTGTCCGGTCATACTGCCCGACCGGATATTAAGAAGCAAATCTCA
TAAACTCCTTTCAGCCTTCTGACTCCTGGAGTAATGAAGGCTGCTTTTTTCGTTACACCCTTCCCCGGCA
GCGCCGGTGGCAGTGTTCAGCCCTTTTTCGGGCAAACTGCTGCCAGTCTGTCCTGGGCGCAACACAGG
CAGCCCGTCGCCATGCTTCTGCCGCTTCCATCCAGCGTCCACTGACTTCCAGAATAACGGCCCGCCCGCC
GGCACGCTGGTAACGCCAGACCTGGCCGTTCCGTGAACTGTTTCTGAATCCATTCATTTTCTTGTCCCCC
TTTAGTTGTCAGTATTATTGTGATAAATAATTCAGTGGACTGGCTTCCTTTCGGAAGCGGAAGTTATTTC
CTTTTAAGCCTTATTTCCCTGCCTCCTCTCTGATGGAGGCTTTTTTTTATTTCTCATCAAGCACGGCCTGA
AGTTCTGCCTTTATCCGGTTCTGGCGGCGGATTTTATCCAGACGCGCTGCCGCATACGCTTCCTCCTCCT
GAGAAATATGCTCCGTCACATACCCTTCCGTGTCATACCGGCAGGCACCGGCTTTCATGGCACACAGAT
AGCTTTCTGAACGGGTGATGGCCTTCAGCGCCCTGCGCAGTTTTTTATGCGAGAGCGGGATATTCCGCT
GCGCCACGTCTTCCAGTAACACGTCCCGGATACCGCAGGCCAGAAGCCGGAGGGTGTCACCGTCAAAT
AATCCCGGCCACCAGGGTTTCAGGGTATTCACGGCCTCATCCAGCGTGGGCAGGTTCAGATAAATGGA
CAGTGCCTGTCTGGCCTGCGCTTTTTTTGCCGCCAGCTCTGCTTCCCGGGCTGCTTTCTCGGCCAGCTTCT
GCTTTTTCACCTTCCACTTTGGTGGCGTGGTGACATTGATGATGGTTTTCCGGCTGCGGACAGGCGTCTC
CCCTTCCGTTTTCCGCTTCAGTGTCAGTACCGGTCGCTTCTGCTCTGTCATGCTGTGTTCTGCCTCTGACG
AATCATCCTGTTACGGTCAAAGATACCACACCCGTCACAGCGCCAGAACGCCCAGCACAGCCAGATGAC
AGGCGTAGAATACCGGGAAAAAATCCCCGGGCCAGAAACGGGGTAATGATTTTCCTGCACACGACACC
AGACCAACGGTCAGCACCGTCATCACAAGACCGGCCACCGCCGCTGCATCACTGGTGGCAAGGTTAAG
CGCCGGAATAACAGCCAGCAGGCAGGCGACCAGCGCCAGACGTTCCGCTCTGTCTTCCGCCCGGTACA
GTCGGTGGCTGACTGCCAGCATCAGCAGCCCGGCAATGCCGTAACTGGTGCCGGACAAAGGCCCCCAA
CAGGGCCATCAGAAGAATGGCCGCTGCTGTACGCCACCCACTGCGCGTTTCACACCACGTCAGCACCTG
TGCCGCCACTGCAAAGGCAAACAGGATATTCCCCTCATACCAGGGAAAGCCTGCCAGATAATATGCGA
ACTGGGCAATAATTCCCCATCCCCACAGCCGGTTAATGGCCGGTTGCCGGATATGCGCATGACGGGAC
AGATTCAGCCCCCACACCAGGGCAAAAAGCGGAAAGGCTCCTCGCCCGGCCAGAAACATCCATTCCTG
CTTCAGCTGGAATATCAGATTGATGTGGTCCAGTACCATCAGCACCAGTGCCACCGTTTTGATGATATCC
CGCTGTCCGGGTGACCAGACCAGAAATGACTGCAACCAGGTATCGGTCCGTGCAGCCAGTGAATCGTT
ACGTGTCGTGTTCGTGTTATCCGTTGTCATCAGTGTTTGTCTTCCTGTATCAGTCTCCGCCCGGGGTTTTC
TCTTTCTGCAGGTCACGAACCATATCCCGCTCCATCTGCTGCAGACGCTCCTGCAGCAGATTTTCCCGGG
CAACCTCCCGGACCGCTTCCCGCTCCCGTTGTGATTCACGCAGCACACTCTCCGGCAGCGCGGCTTCCC
GTTCAGTTATGGCAGCCCGGTCACGCTCCTGCCCGGCAATTTCCCTGACAGCCAGCTCTGTTTTGCCGTC
CGGCAGGTCAGGTTTGTTCTCTGCCATTTTACGGACAATTTCATCTGCGCGACGTTCCGTTTCACGGCGG
ATGGCCTCTTCAGCCTGCCGCTGTGCCAGTACCTCTGCCGTGACCGGTTCGCCATTTCCGGCTCCCGGCT
GGACGCTGTTGTCCGGGATATCCCCCCACACGCGGCCACCGGTGATGGCACCGGGATTCCACGGACGG
CCTTCACCGGCAATGCTTACCACCACACCACTGTCAGGATTATCACGGGCAATCCGGACGCCATCCTGC
ATATTATCAGCCAGCAGGCTTTCTCCGTTACGGCTGCCCTGCAGGGCCACGAACTGCGCATCCCCGCTG
CCTTTTACCCGACCTTCACCGCTGAATCCCCGCAGCCCGTTTCCGTCATCCGTGGTCAGCGGGTTCAGCC
AGATACCGGCGGACTTGCCGTTACGGTCAAACGCCGGCAGTGCCACATACGGTTGCGGATATTTACGT
CCCGGAGCAATAAACCGTGCAGGACTGTCTCCCCCGGCCAGCCCCGCCTGACGGAGAACGGCACGGCC
TGCCGCCACGTCCCGCAGCTCCCGCGCCGTACTGAACAGCCGCTCTGCATTCATGACCTCCCGGTCCGG
TTTCGGCTCAAACACATCGTGGGCAGTTCCTTTCTGTACGGCATTGTTGATGGCATCCGTCCAGCCCTGA
CGGTTATCGGTGTACACCTGCACATGCTGCTTCATACGCGACAGGGCCACGTAGGCTGACTCAAAGCC
GGCCATCAGTTTCCGGTTACCTTCCGTGCCTTCAAGCGCGATGGCAAAGGTTTCACTCGCCCCCTGTGC
ACCGTGGGCGGTGATGGCATAGGCCAGGTCAATATGTTGCTCTGCCCGCTCCTGACCGGGGCGAATCA
CACGGGTCTGCTGACCGTCAGAAAGTGTGACACTGTCACCGGAAACCGCTGTCACCGTCCAGACGCTG
TTGGCCACATAACCGCGCTCCCGGTCACTCTTCGTGAAGCGTATCCTGTCACCGGTCCCCACCCGGATG
GTGTCCGGGGTGTACAGCGTGACGCCTTCAGCCACCGCCTCCCGGGGCGAAATCAGCCGCGTGTTACC
TTCCGCATCCTGCAGGGTTATCAGCCCGTCATCCTTGCTGATACCGGCAATCCGGTGATACACATTATCC
ACCAGGGCAAGGGCATCCGGGTTATTCTCCCAGGTGGAGAGACGACGCAGCTCCCCGTCGCGTATATT
CGCTGTGTTCAGGACAGGCACCATGACCTGCACCTGCCCCAGTTCTCCGGCCTTTTCCCGTGCATCATGA
ATCATGCTGTTCAGTACGCGCCGATCCTCATTCAGGTGCGTGACAATCAGCGTCTGCTCCCGTGCTTCCG
GTGTCCTGCCGGTATAGTCGCGGACAATGGCTTCATACAGTGTCATGGGGACATCCGGAAAAGCCTCG
CCTTTCAGCATCGCCTTCTGCTGCGCTTCTGCCAGTTTCGCTTCCTGGCTGTGACTGAACTCCGTCACGG
AGTGCTCCGGTGCCCATGCGCCCTCCAGACGTGGCACCTGAGACGGTTTCACACGCTCAAGCCCGGAC
AGCGCCCTTTCCACATCCCGGTTAATCAGGCTGTATACCGCCTCCCGCAGTTCCGGCGTCTGACGCACA
ATCTCCTTCATGATGACCACATCGGCAGCACTGCGCGTCTGCTGGAGACGGAAAGGCTGACCAGGCGC
GATGGCCTGCAGCTGGTCCGTGTCACCGCTGGCCACAGCCCGACCGCCACCGACCGCAATCAGGGCGT
ATGCCCGTGCCATGTCGGTATTGCCCACCATTGAGCTCTCATCGAGCAGGAACAGCGTGTTGCTGAAAT
CCGGCGTTTCTCCGCTGCGCTGCTGCAGCTGCGTGTCATGCAGAAAGGACGCCAGTGTCTGCGCATCCA
CGCCGGCGCTGCGCATCTCCCCGACCGCGCGGTGCGTGGGACCCAGCCCAACGACACGGGGACGTTCA
CTCTCCGGCAGCATGTTCACGGCTGACATCACCGCCCGGAACTGTGTGGTCTTACCCACACCGGCATAG
CCCTGCACCACCGTGAAACGGTCGGACGTTTCCAGTATCATACGGGTGGCGGCGCGCTGTCCTGATGTC
AGTTTCTCCATGAGTTCGCCAGGTACTCTCTCCATCAGCGGCGTGACCGCCTCCTTACCTTCGAGAATAT
GGCGAAGAATGCTCTTTTCTGCCTCATACGACGCACGGGAAACCAGCAGGCCTGTGCCATAGCCTTTTG
CCACATCCACATACAGTAAATCACCCCGTTTTATCTGCGCATTGATTTCCCCTCCCAGGTCAGCAAAACC
GGTTCCTTCTGCAGCAAACGACTTCGCCTCTGTCAGCAGGTCCACCATGCTGAAGGCCAGGTTTTTACTT
TCCAGCACAGGAAGGGCCAGATGAATGGCCTGCTGTGCCGGCGTGTGAAGCCCGGCTTTCTGCAGACT
GATAGCGGTTTCCAGCAATGTCTCACCGGCACGCGCCTTTATCTGCTCAGAAACCACCGTAAAGGAGG
GATGGCGGGCAAGTTTTTCCGCAGTACGGGTTTCATCCAGTGAGGAATACAGCCGGACATCACGACCA
CTGCGGGCCAGACCGTTCAGGGTGGCATTGTCCATTGCCATCTGTGTGACGGAGGCAAAAACCGTCGC
ACTGTCGCTGACGGAATGCCCGGGCGTTTCCACCCAGCCGTTCTCCAGCTTCAGTGCCGTGAACGGTGA
ATCGCTCACAGGCAGGGTGGCCGGTTCAGCCCGTCCCGGCACAACAACCGTCATCGCATCTTCACTGAC
GGATGCCACCTGCAGGCGGTCACCGCCGGAGACGCGGAGTCCGGGAATTTTCCCTGTCACCCTCAGAC
GCTCACCGTCTGCCACCGGCATTTTTTCCGGCCGGAACAGCGACCAGCTGCTGTCCAGGGAAGAAATAC
GCACCACCTGCGTTTCGCCCTGCGCATTCCGCAGGGTCAGGCTGTGACTCTGCGCCGTCACACGTTCTG
TCACATAGCGGTCATGACTGCGTGTCTCCGGGTTCCACTGCTCCATCACCATTCCCGGGCGGTACATATC
CCGCAGATAACGGCTCCGGCTGTCCAGCCAGACCGGTGACAGGGCGGTCATGGTTACCTCCGGGTGTC
CGAGCACGCCCTGTGTTTTCAGCTCACTGCGAATGGCCTGTGTCAGTATGGCCTGTTCCCGTACTCCGCT
GACCTGTGCCACGCTCTCTTCTCCGGCTTTCACGCTGGCCGCAAAATCTCCGGCCAGCCGGGCATAGCG
GACATTACGGTCCGGTTCACTGATGATGGTGGCCGGTCGCTGTTCTCCCCCCTGCCAGCGATATGTGTT
CACCCCGGCATCCTTCATGGCCATCAGTGCACTGCCTGTACCGGTTCGCTGCCCGCTGTCGGTTATCAG
GACCTGTACGTTATGACGTGCGGCACCATCCAGCAGGGTTAACGTCTCTTTCAGGGAGAGTTTTTCGCC
CTGGTCGACGATAACGGTACTGCCCGGCGTGAAGGCCATGCCTTCCAGCAACTGACGACGTCCGGTTA
TCAGTTCACCGGACAGCCGTTCATCCTGCTTCAGGTTCATCTGCGAGCGACGGTCAGCAGCGATAATCT
GCACCTCCCGCCCCTGCTCCCGGGCCATCATGACCAGTTCAGCCACCCGCTCACGCTGCCCGGCTGCAC
CGCCCTGCCCGGACACAATGGCCAGCGACGGACGGTCCTGTGCCAGAACACTGACGGCATCGCTGTAA
CCGGCCGTCCGGGGGACACTTTTCTCCGGATGTACGGTCACCCGGTTCTGTTTCATGATGTCACGACTG
AGTGCCCGGACTGACAGCTCATCGAGCATATGAATCCCGGACGTGAACAGCCCCTTCTCACGATCGAG
GGGGATAAGCTGCTCACGGCTGATGGCCTCATCGATACCGGCGCGTGCCCGTTCAATCACACCATTTTC
CGGCGGCAGTATGCCGACCGTCCTGGCCAGCAGGTCCGTATACATAAACTGCACTTTGCGTTCACTTAA
TCCGGCAATGGCCTGTGTCACCGCCTGCTGCACATCCGGCCCGTCCTGTGAAGCGGGTCCGGGTGTCT
GCGTGCGGGTATATGCCCGTTGTTCCGCTGCGTCACGATATGCCCGGATGTCGAACCCGGTTTCCTTCA
GCGTCTGCATCCACTCGGCCATTCTGACTTCAGGATCGACGTGCTGTTTGGATTTACGCGTATCCAGCG
CCGCCACATCCCGGGATTTCAGCGAGGCGTCTTCCCCGACGGCCTCCCGGATAGTCTGTGAGCGTCCGG
AAAAGGCCTCCACCGGTACACCCGGCATTTCCCACATACCGTGCTTACCGACCACTTCAGTTTCATAGCC
CAGCGCCTCAACCTGCTCTTTCCGTTTTTCCCGGTAGAGCCTGCCAAAGGCAATCTGATTAGCGTACACA
TTCTCAATGAACCCCGTTTTCCCCACTTTGTCACTGCTCAGTGTCTTCCATTCGCCGTTATGCTGCGTAAC
ATTAGTCACCACCGCATGCGTGTGTAACTGTGGTTCCTGATCGCGACTGGTGTCGTGGTTAAACAGTGC
CATCACCAGATTACCGGTCAGCACCGTTTCTGACTGTCCGTCCGTCATCACCCGTGTGGAGGCCAGTGC
CTCCACCTGACGGACAGCAAAATCCACGGCCTGGTTATGTGCTTCAATAAGACGTTTGTCCCCCCCGAG
CATGGCCATCATGGAGACACTTTTGGGAGCGGAGAAGGTCAGGTCGTAGCCGGGACGATGCCTGTTAC
TGCCATCCTGCATGCGGCTTAAATCCGCCCCGTCCGGCAGCCTGCCCTCCAGAAGGCGGGTAAAGACAT
CCTTATCGACACTGCCCTGCAGCCCCAGTTGTTCAGCCCCCTGGCCGGCCCAGCGTTCTCCCATGCTGCC
CAGCACATAGTAATTATCCTTGTCGGTATAATAATTATCGGCACTTCCGGCCGATCTGACCTGCGCGATA
CTCATCATAAACGTATATCCCTTTGACATATCCCGGTATCAATCCCACAATAGATATACACAAGACATAT

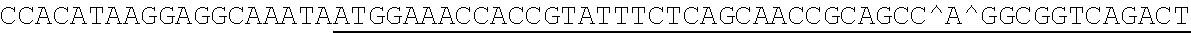

GCCAAAAGCGGTTGCATTGCCGGAAAACGTAAAACGCGTTGAAGTGATTGCTGTCGGACGAACCAGA
ATCATTACGCCAGCCGGAGAGACATGGGACGAATGGTTCGACGGACACAGCGTCAGCGCCGATTTTAT

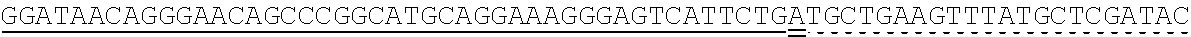

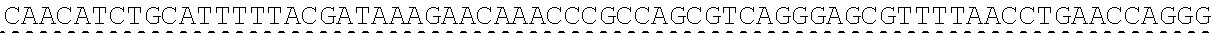

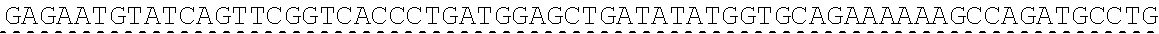

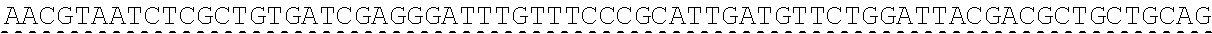

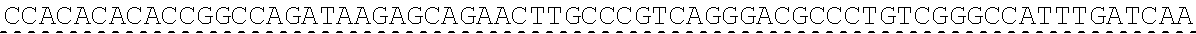

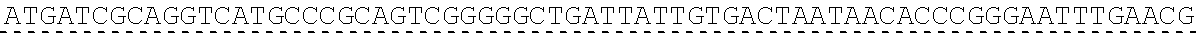

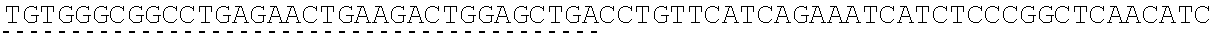

CTCCCCGCGTTCCCGGTGCACATTGATGTTCACCTCTTCACGACGCTGCATCTGCTGCCAGGTGTCCGGA
TGATTTTCCTGCTGCCATGCCTCATAAGCGGCCATATCCACCACTTCACCGGATTCACTGATCCCGGGTG
GCAGTTGCTGTTCCATCTCTTCTTCCGGTTTCATTTTCAGCTCCTGCTCAATCCCCCCTGCCGGAACATTC
ACACCTGAATCTGACTTCTTATCGTTGATGACAGGAGACACCGGCTGTTGCGGCTGTTGCGGCTGTTGC
GGCTGTTGCGGCTGTTGCGGCTGTTGCGGCTGTTGCGGCTGTTGCGGCTGTTGCGGCTGTTCAGCCTG
AGTCACGTCTTCTCCGGAAACAACCTCCGGGACATCCGGTTCGAAGAGGCTGGCCATCTGACGACCTTC
TGCTTCCCTTGCGGCAAGTACGGCACTCAGACGGTTCTCCATTTCCGGGTTGATGTCACGCGGAATAAA
CTCCGGAGCGACCTTCGGACGTTCCTGATATTTCAGTGAGAGTTTGACTGCCGGATACGGTCCGGGCA
GGGTGACATAGCAGGTCAGATCCGGCAGGGACTGAATGTCGGAATAACTGACCAGCGTCTGGCGCTC
CATATCCTTACCGGTCGATACCCCGTCACGTACCGGATCAGCACCGTAGGAATACTGTTCGCTGGCTTTC
AGGTGCTCTTTCTCACCAATTTCACCGACAGCGAACTCCGCAATCTTATGGCTGGGAGAACGGAAAAAG
GCACGGGTGTTCATGACGTCAAACAGCGTGGCAGCCGCTTTCTCACCGTAGATATCTTCCAGCTGGGCA
TAGGACTGGATACCAAACACATAACAGCCACCGAACTTACGGGCTTCCGGCAGGATCTCCACCAGGTC
CGGCAGTTTGTGTAATGTGGGTAACTCATCACAGAAGAACCACACACGACGATTACGGTTTTCTCCCAT
TGCCAGCAGACCACGAATGGCAATGGACAGCCACATGGAGATCACCGGCTTCAGGGAGGCATGAGTG
TCGGCATTCGACGAGATAAACAGCCAGCCGTTTTTCTGATCCTCCCGGACACCACGCATCCAGTCACGG
ATGGTGAAGGGCTCACCGTTATGCTCAATCCCCTGCAGGTAACGGATGGCTTTCACATAGTTGGTCAGC
ACAGCACGGATGGAAATCGCCGTTTTCTCAATTTTCTCTTCCACCAGGTTGGCCGCCGGTGAATTACGC
AGGTAGGTACGCAGTTTTTCAATTTTGATGGAAAGCAGTGTGTCCACCAGTTTGCTGTAGCTGCGGTTG
GGGTCATTACGCATCAGATACGCCGCTTCCGCAAAAATGGTACGTCCTGAACCCTGCCAGAACGGGTCC
TCTTTTGTGCCCATCGGGATCAGAGTATTTGCGGTATTATCAAAATCCGGCTGTGTCAGGCACTCCTTCC
ACAAATCCCAGGCGGCACAGCGTGCATCCAGCGGATTCAGGATCTTATCGATGGAGGGATCATAGTAA
CTTTTAACAAATTCCCCTGAACGGTCATAAATCACCACCATATCACCACGCTGACGGGCGTAGTTGGCC
AGACGACGGATAACCTCCGACTTACCGGCCCCGACCGTGCCGTGCAGACAGAAGTTCTGGATTTCGGA
ATCCCGGATAATCGGCAGATCGCCAATCCGGATATCGGAGTCCCTGCCGTCTTTTTTCAGCATCCGGGC
AACGTCTTTCGGATTGTCTGTCAGTTGACGACCACCTGTGACTTCATTTTCGCTCTGTTGTTTACCCTGAC
GCCCCAGAATCCAGGAGACAACAAAGAAGGTAATCAGGCAAATAACCAGTGCCACAACTGTGGCCAG
AACGAATGCAGACCATAACTGCTCACCGCACCAGATCATATATTTATCATGCAGTACCTGAGCAGCGTT
CATCCGGAAGGTTTTGCCGTAATACTGAATCTCATATACCGGCTGAGACTTGATTAAATCCCGCATGCCT
TCCAGCGTGGTACACCACCAGTAAATACAGCCATTCACAAACGTCTGCCAGCTTATTTTTACCCATAAAA
CCAGACCAACGAGTATCCAGAAAAAAATAAACAGGCAGTAAAGCATAATATTGGCGATCTGGCTGAAC
ATGCGGATACGCGGTAATGGCTCCAACTTATTGATAGTGTTTTATGTTCAGATAATGCCCGATGACTTT
GTCATGCAGCTCCACCGATTTTGAGAACGACAGCGACTTCCGTCCCAGCCGTGCCAGGTGCTGCCTCAG
ATTCAGGTTATGCCGCTCAATGAGGTGTACTGGCAATAGCGGACACTACCATTTGTTCTTTTTTTAAGCA
GCCATCTGATGATATTTTTCCCTGAAGGCTGCCGGGGAGATATTCCCCAGACGAGAGTGACGACGCTG
ACGATTGTAGAAAATCTCAATGTATTCCCGTATTACTGAGATGGCTTCATCCCGGTTATTAAAACGATAG
TGGCTCAGGCTCTCATTTTTCAGCGTTCCCCAGAAGCTTTCCATCGGAGCGTTGTCGTAACAGTTACCTT
TACGCGACATTGATGTTTTCAGACCAAACTGCTCCTGTATGACCCGGTAATCGTATGCGCAGTACTGTG
AACCTCGATCAGAGTGGTGGATTAGCCCGGCAGGTGGGCGCTGGCTCCTGAGCGCCATAAACAGGGC
TTTACCTGTCAGCTCTTTTGTCATGCGCTCTCCCATGGCGTAGCCGACAATTTCGCACGTATAAACATCTT
TGATGCCAGCGAGGTACAACCATCCCTCCTGTGTGGCAACATACGTCAGGTCCGCCACCCAGACCTGAT
TTGGTGCTGTAGGAGCGAACGTCTGGTTCAGCAGATTTGGCGCAACTGGCAGATTGTGGTTCGAGTTC
GTAGTCGCTCTGAACTTGCGTTTCTGCTTACAGCGTAGCCTTAGCTCCTTACGAAGACGTGCCAGTCGG
TCACGACCAACGATGATGCCATTCTCTGCCAGCTCCGTCTGGAGCCGCCGGGTTCCATATGTTTCGCGA
GTGCGGATATGTGCCACCTTAATCTCCAGTTTTAGCCGCTCATCACTTTGTTTTCTGTCTGAGGGTTCAT
GCTGTACCCAGTTGTAATAACCGCTCCTGGATACACCAAATACCTGACACATCGCTTCAATGGGAAATT
GTTGTCGCCATTGTTCGATTAACGCGTATTTTTCAGCGACTCCTGTGCAAAATACGCTGTTGCTTTTTTTA
ATATATCTCGCTCAAGGCGAGCTTCATTTAACGCCTTACGCAGTTGCAGAATTTCAGATTCCAGTTCAGC
CACCGTGCGGGAACCAGGAGTACCGAGCCCTTTTCTGGCGGCGGTAACCCATTGTCCTAAAGTGCCTTC
AGGAAGAGATAATCGGGAAGCGCCTTCACTGATCGAAAGTTGATTTTCAGGAACCGTTCTGACAGCTT
CGGCTTTGAACTCTTTAGAGTAACGTTGGGTTTTTCTGCTCATTATTAGCTCCTTCTGATGCCATTCTATT
TCAGGAAGGAGTGTCCGTTAAACTCAGGCTACCTCAGGGTCAGTCCTGCATCCCCGCTGTCGTGGTACA
GCCACAATTGGCGGTTAATCACAAGAGAAGTCAGAATGATGCCTGCGGCATCAGCACTCACGGTACGG
TCAAACCAGTTTGCACCGTTCACCATGTGGAAACGGTCGCCATCCGGCATCATGTAACCGCCACCGTCC
GGCAGGCGGATGAAGTGCCAGAAGCCGCCCTGATATTCCGGCATCATTTTTTGCGCCAGCGCGTACAC
ATACGCCTCGGCATACAGAAAATCGTTGCCAAACAGGTAAGGCAGAAAGGAGAGACGGTCGCACTCTT
CAACATTCAGAGTGACAGGTTTCGCATATTGCATGATGTTTTCCTCCGTTGTTCACATATTCCGCGACAC
CGGGCGGTGCCGTGCCTCCATCTTAGCCGGGCGATAAGGCGGGGATGCACAGGGCGCAAGGGAAGCC
GCAGCCTGACGCACACGGCTGGCGGGAGTTGGAGGGTGAAACCGCGAAATCTGAGCGGGTGAACGA
CCGGAAAATCACGGCAGACGGGAAGGAGGGACCGGGTGACCGCAGCGGCGAAGCGTACCCTTGCAT
CAACGCCAGCCCGGCTAAGATGGAGGCACGGCACAGCCCGCACACGACGCTGAGGCTACAGAGCTGA
AAACCCACAAGGCCATGCCCCGCATGGCCGGGTAAAGCGGAGCCGGCGGCGACTTCCTTTGTGCTCTG
TCCGGGGACCGGCAGGAAAAAGGCGGGGATTTACCCCATAAGCGCTAACTTAAGGGTTGAACCATCTG
AAGAACGCGACGCCTCGGTGCCTCGTTAAGACGATGCCTCGCGTTCTTCAATTGCGTTTTGTAGGCTGT
CAGGGATACTGTCCCACGAATGGCCACCTGTAAGCTCCAGATGACCATTTTTGTTATTCTCCACAACGCG
TTAGTTCTTCTTTTCGGATCCGGCACTTCTGGGGGGAAAATCCAGCGATGGCTGGATTATGTCGTCAAT
TAAAAATGCGGCGAGTAGATTAGCAAATATCCACGCTTTCGCGAGTTCAAGTTCCTTTGCACGCAAAGC
ATCCAAGTGCAGCAAACTTTTGAGCCGCTTAAAAGCCAGTTCAATTTGCCATCGCAGACGGTAACAATC
AGCCACTTGCTCTGCTGAATATTCATCTTCCGATAATGATGTTAGCAATAGCACATGGCCCGCTGCCTCC
AGCGTTTCCGCCTGAACTACTCGTCCTTTTCGACGATTCTCGCTGAGCAGTCGGGTTTTACTGATTAATG
CTTTTTCGGGAGGAAGTGATACGGCAATGAGACGTGCAGGAAAGGGAGCTCCAGCTTTTTTATTACCT
GAATTGCCTATCATTACAGTGGTTTCACCGTTCTTACCGCAATCCAGCCCGCGCAGAAAATCCATCATGT
CAAAGCGCATTCCTTCTGCAGTTAACCAGCGCAATCCTCGCCAGTAAACCCAGACGATATAATCAGCTT
CTCCAAAAGCAAGTGAGCGGATACATTCGGGACGCGAACCGAATCCCCGGTCAGCAATTCGTATCTCG
TCTGCCGTTTGCGCAAATCGATCCAGCCGTTCAGCGTCTCTGCTGTCGGTTAGCTCAAAATCAGTGAACT
GACAGGTATGAGGATCATATCCCATATGTAGTCGCCATTCAGCGCTGCCGCCCCCGGGCGCGCTGATTG
CTGTTCCATCGACAAGACGCAATCAAGGTGCGGCGGGAGTCGATGGCATGCGCGTAAGTGAACTGCC
GGACTACCTGAGGCACCACTGGCCAGAGCTGAAAGCGCAACTGCTGTCCGGCAGATACCGCCCATCCC
CTGTGAGAAGAGTATCCATACTGAAGCCCGGCGGCGGCGAACGTCTGCTGGGCATCCAGATGGTGGT
GGATCGCTTCATCCAGCAGGCTATGATGCAGGTATTGCAGGCACTGTGAGATAGCTCGTTCAGCGACA
ACAGCTACGGGTTCCGTCCCGGTCGTTCGGCCCACCAGGCAGTGATACAGGCCCAGGAATATATCGGG
GCCGGGTATCACTGGGTGGTCGACCTCGATCTGGAGAAGTTCTTCGACCGGATAAATCACGACGTGCT
GATGAGCCGGATAGAGAAACGAGTATCGGATAAACGGGTGTTGTCACTTATCCGCAGGTTCCTGAATG
CAGGGGTAATGGACGCAGGTCTGGTAAGGCCGGTGACAGAAGGAACGCCGCAGGGCGGCGTGATCT
CGCCCCTGCTCTCAAATCTGTTCCTGCACTATGCCTTTGATATGTGGATGCAACGTCAGTGTCCCGACGT
TCCCTTCGAAAGGTACGCCGATGATGGTGTGCCACGAAGGCGCGCACCAGGGACGTGAGTCCCTGTGC
AGCCATGCTGCACAAGAGATGAGGGAAGGCCCCTCGGTGTCGGCGTGCAGGCGCAGGCACCAAACCA
CCCACTGCTGCTGCATTTAAGAGATGTGGTGGTGAGCGAGATGGGAAAAGGTCGGACGTGAGCCGAT
CAGGTATGGATCGAAGAGACGAACGAAAGTGAACCGCTGATGATGTGTCGAAAGCATATAGATGATG
TCAAAACCATGGTTATCCGGCTCCGGCGTTAACGTCACGGGAAGCAGTGCCGGCATATTTTTTCCGGAA
GGCAGCAGGCCACCTTTCCGGTATTGATGGCGCCAGTTGAAGAGCAGGTTATCGTTGATTCCATTTTCA
CGGGCAATCTGCGCCACACAGGCTCCGGGCTGCAGTGACTGCTCCACTAAGGCGATTTTGAACTCATAA
GGGAAGTTGGGGCGCCGGGGACGCTTTTTTACCCCGGGGACTTCGGGCATAACGGTACTTTCAGTAAG
TACGACAGGTACTGTGGAGGCACTCCCGTAAAGACGGGCGTCAAGTTCCCGCTCCGACATACCTGCGG
GCAGAGGCCATGAAAGGCCAGCTTTGCGGAAGCGCACAAACATACTACAAACTGTTGATTTTGGTACG
CCCAGGCGACGTCCGGCTACAACGAGGGGTAAATGTTCTTCAAAGTGAAGGCGTAAAGTGTGGATGG
TGCAGAAGATGCCGATTTCCAGCCCCCGTTTTCGGGCTGCATACAGCAGATTCTCCACCGCCAGACGAC
ACACGTCATTGCAGCCGTCAAGCTGCCGTGGTTCCGTGCCGTTCTAAATAGCTGCGCGGAATAGTAGAT
CACTTTGAGGGAACTTAGCCCGGATTGTGCGATCTGATCAATCGCCAAATCAAAACAAATCACCAACCG
GACTGAGCAATGCCGATCATAGCACCAATTTCCCGTGACGAACGACGCCTGATGCAGAAAGCCATCCA
TAAAACACACGATAAAAATTATGCCCGCAGACTGACTGCCATGCTGATGCTGCACCGGGGCGACCGTG
TCAGCGACGTTGCCAGAACGCTCTGCTGCGCCCGCTCCTCTGTTGGACGCTGGATTAACTGGTTCACGC
AGTCGGGTGTTGAGGGACTGAAATCATTACCTGCCGGGCGTGCCCGTCGCTGGCCGTTTGAGCATATC
TGCACACTGTTACGTGAGCTGGTAAAACATTCTCCCGGCGACTTTGGCTACCAGCGTTCACGCTGGAGT
ACAGAACTGCTGGCAATAAAAATCAATGAGATAACCGGTTGCCAGTTAAATGCCGGAACCGTTCGCCG
CTGGTTGCCGTCTGCGGGGATTGTGTGGCGAAGGGCTGCGCCAACTCTGCGTATCCGTGACCCGCATA
AAGATGAAAAGATGGCAGCAATCCATAAAGCACTGGACGAATGCAGCGCAGAACATCAGGTCTTTTAT
GAAGATGAAGTGGATATCCATCTTAATCCCAAAATCGGTGCGGACTGGCAACTGCGCGGACAGCAAAA
ACGGGTGGTCACGCCGGGACAGAATGAAAAATATTATCTGGCCGGAGCGCTGCACAGCGGGACAGGT
AAAGTCAGCTGTGTGGGCGGCAACAGCAAAAGTTCGGCGCTGTTCATCAGCCTGCTGAAGCGGCTTAA
AGCGACATACCGTCGGGCGAAAACCATCACGCTGATCGTGGACAACTACATTATCCACAAAAGCCGGG
AAACACAGAGCTGGCTGAAGGAGAACCCGAAGTTCAGGGTCATTTATCAGCCGGTTTACTCGCCATGG
GTGAATCATGTTGAACGGCTATGGCAGGCACTTCACGACACAATAACGCGTAATCATCAGTGCAGCTCA
ATGTGGCAACTGTTGAAAAAAGTTCGCCATTTTATGGAAACCGTCAGCCCATTCCCCGGAGGCAAACAT
GGACTGGCAAAAGTGTAGCGGTATTAAGCGCAGCTATTTAGGCATAAATTAAATTTAACGAACTGCATC
TACACAGTCCTCCGTGCCAAACGGGCAACACACGGAAGGGGGATGTGTTTCGATTGGTAACCTGTAAG
AAACAACTAGTGGAAATACCTGTACTTTATAATTTCAAGGGTACGGGTCCGCTTCGTTTGATTTGCGCGT
TTTTAACAATTTTAAAACCCAGATATACTGTTCCGGATGTGACCCCACCAAAATTTCCACTGTTTTGTTCA
TTTGTCGGGCAATCATTTTATTGTCCGCGCTGGCAATGCATGCATCCATTGGTGGCCGAACTTCAATAGT
CAGGAAATGTTTTTTTTCATCATAAACCGGGAAAAGCGGTATAATCATAGCCTGACTGATGTTCATCAG
ACGTCCAATAATTGGTAATGTCGCTTTATAGGTCGCAAAAAAATCAGCAAATTCACTGTATTCAGGACC
ATGATCCTGATCTGGAAGGTAATACCCCCAGTATCCCTGGCGTACTGACTGAATAAATGGTTTTATCCCA
TCCTCCCGGGAATGTAAGCGCCCCCCGAATTTACGCCGTACAGAATTCCAGACATAATCAATCACTGGA
TTTCGTTGCTGATGAAACATGGCGGCCATTTTTTCCCCCTGAGCTGCCAGCAACATTGCAGGAATATCCA
CGCTCCAGCCATGGGGAACCAGAAAAATCACATTTCTGTTATTGTGCCGGATTTCCTCAAGAATTTCGA
GGCCTTTCCAGAACACTCGTTTCTGGAACTTTTCCGGACCACGAATTGCCAGTTCTGCCATCATCACTAT
GGATTGCAATGCTGTGGCAAACATATTGTCGACAATTATTTCCCGTTCTGTATCACTCTTTTCCGGGAAA
CAAAGCGATAAATTAATCGTCGCCCTGCGACGAGCTTTTCTGCTCAATCTGCCCACCCAACGCCCTATTT
TGGCCAAGACAGGATCTCTGAATGAAGGGGGAAACATTGAAATACCTGCAATCATTCCCAAAACGAAC
CATGTAAACCAGTAAACAGGGGAAAGATAATTTTTCTTAAATTCAGGAATAAACTCGGATTTGTATTTTT
TCATTATTTAGTGCGAATGCCCCTACCAAAATCATCATTAATATTTTATTCAGAGAATACATCAAACCTTA
ATTTAAGTCCTGATGTTCTGAGTTTACAGGATATGCATCCAAAGAGCGGATAGCAGTTTCATGGATATA
ATCCAACAAAGCGTATCGTTTTCGATACTCAGAGCGTTTTTTACTAGCTATATTGCTTAGCGGCTTTCGA
ACCACCTGTGTTGGTAATTTGTACCAGTCACCACAGGTTTTCCCTGCTACAGACTCCCAGAAATCACTGT
AGACAGCAACAAAGGTTTTGCGTTTCTGATACCAGTATCTCAATTGCCGGAAAACATGACTGTGCTCAC
TGACTGCCAGACACTCAGAGACATTACAAGCTTTCATCAACGCACAAAATGCTTCAAAAATAATCCGTTT
TGGGAAAAGCCCATAACAGGCCCGGGTTGCGCACCGAATAACATCAGGTCCGGTATTTTTGGGGGCTC
CCTGTAATCCGCCAATAAAAGCGGTGTGTCCATTTTGAGTCAGAATGACTGAAAATGTTAACCGGGCAA
TCACTATTTTATTGAAAAACAATGACAACATCAACTCTCCTTCCCGGTCAAAACCACTGGGAGAACACTG
GATATCAAAATCCTCTCCATCTTTCCCGATGAATTGGACTAATGATACCGATTTTTGTGGAAACAAAAAT
TCAGAATATTTTTCTGGCAAATTTTGCACAAAGCGATAATGCTCAAGAATATATTGCCCCCGGGACCAG
GCTCGCCCCCCTTTATGCAAATAAGGTCGATGAATTCTTGCTGGCAACGTGGGTTGTATTGCCAGAATA
TCCTCAATGCACTGCAAGGAACGCAGTTCGTAATAATATCGAATACTGGCAACCGGATTTATAGCACAT
CGCAAAATGTACATGAACCGGAATTTTCTGTGTTCCCACTTCCCCGGGAGTGAATCTGAGGAAAAAACA
ATCCTTTTAAGGAAACCGATAAATGATAAGTTACTTACAGAAAACATATTGTTACTTGTGCTTCGCTAAT
GTGAGTATTTTTAACGCAGTTTTATTAATATCAAACCGCTCTTCTATATCCTGTTTTGCCTGGCGAGCCAT
CTGATAAAAACGCCCAGGGTCATTAAAAAAATCATTCAGGGCGCACATCCATGCGTGCTTATTACCTGC
TGGTAACAATGTCCCGGTCTGGTTGTTCTGAATAACATCAGGGATTCCACCAATCTGACTGGCCAGTAC
AGGCACAGAAAATGCCGATGCTTCTGCCAGTACCATACCAAACGATTCGTTTTCTGAAGGCAGAACCAC
CAGACTGGCAACCCGATATACGGGGGGGGCAGGAAAAACATTGTCAGCAATAAAAACATCATCATGCA
TGCCCATACTGTCAATCTGATACTGTAAATGCTCCCGCAGTTCAGGCGAACCAGACCCCACAATCAACC
AACAGAACTGTCGTCCATTCATTTTTAAATGGAACAGTAAATTCAACATAAATTCGTGCCCTTTTTCCGG
GCGCAACATTCCTACCTGAGCAATAACGGGGCATCCCCTTCGGGACGCCAGCCAAGAAAGAACATTTG
GAGGCAAACTGTTTCGGGAATCAACGTATAATTTCTGGAAATCAAAGCCTGGAGGCACAACAGTAACC
CGGGTTCGACACCCTTCCTGCTCCAGATGTGTCTTCATACTTGTTCCGGGAACAATCACCTCATCGCAAA
AATGATTTATTGAAAAAACTTTTGTTTTTCGTGTCAAATATGTCTTTTGCCTGATAATTCTGAATGGATGT
TTCCAAGTGAATAACCGTACTAAACCAACAATATTGCTATCATGCCCACTGTGACAAACGATTGCATTG
GGCTGAAAACCATGAACTATTCCGAGTAATCTCCATGCAGTAGGGATGTGAAGACTGTTTCGAAATAAC
GCGAATGTGATATCAATTCCCAATTTACTGGCTTCAAAAGCAATTTTGCTGTTTTCCCTGCAGACAAGCA
GAACTGAATGCCCCATTTTCTTCAGGGCCTTCATTTGAGCAACAGCCTGTAATTCCTGACCACCTATATT
CGGTGATGATTCCGTAAATAGTATATTCATTATTAATCATCCGGTAATCTTGGCCCCTTATGAAAGGCCA
GCAACGATGAAAAAAACGGTGTTGTATAATAAAAAAGTCGTCTTTTCAGCCATGCGCAACTTTCCCGTT
CTTTGGTGCTTATTCTTCCTATCCGTGTAGTTCCTTTAGCAGGAGCATTCATTCTTCTTTCTGTTGTATATA
AATAATAGAATCCAAGTTCTTCAGCGACCTGAATATAATCCTTGTTATAATAACCTTCCGGCCAACACAA
ATGTTTACTGCATTTTCCTGTCATTTCTTTCAAATATTCTCTCCCGCTCAACAAGTCCTGCCGAAGATGTTT
ACACTGTTCTTCTCGTGAACTGAACTTITTATCCCAGCGTGTATGAGTATGCGTATGAACATGAAATTCG
ACCAGACCAGATTGCAACATTTCGTTTACTTCAGACCAGCGAAGCATAACATTATCAGCATTCCCTGTCG
CTATTTGATGTTCGCAATCGCGATGAGAATATTCCTTTCCAGGAGAGTGACGAACCGGACCATTCCCAA
TAAAACCGGTAATTAAAAAAATATGTGCTTTGAGATTAAACTCTTTTAATAAAGGATATACCTGAAACCA
GTTGTCAAGGTATCCATCATCAAATGTCAACATAACGCTTTTCCGGGGGAGTTTTCCGCCCCGATAAAA
AAACTCCAGTTCATCTGATGACAGTGTTTTCCAGTTGTTTTCAGCCAACCATTTCATTTGCTTCCGGAATG
TCACGGGAGAAAGTGTAACCAGTCCGGGGCAATGACTGACATGATGATACATTAATACCGGAACATGA
TTGGCTTTGAACAAAATGCCCCCTTCGTTTAACATTTAAAGTTTCTGGCACCTAGTAAGCAGAGACAAAA
GGAAAACCCAATAAAGCTGGTGCATTTTATTTCCAGATACTGGATGAATAACATAGCAAACCCATGGCA
TATTTGATATAATTTACTAAATTAAACATCAACAATTCATAAACAATGATATATTATCATATCTTTTAAAA
ACGACTCACTAAGAACCTTGAGGTGGCACACTGAATTTGGCTACCTGAACAGTGAGGTGTACTGGCAA
TAGCGGACACTACCATTTGTTCTTTTTTTAAGCAGCCATCTGATGATATTTTTCCCTGAAGGCTGCCGGG
GAGATATTCCCCAGACGAGAGTGACGACGCTGACGATTGTAGAAAATCTCAATGTATTCCCGTATTACT
GAGATGGCTTCATCCCGGTTATTAAAACGATAGTGGCTCAGGCTCTCATTTTTCAGCGTTCCCCAGAAG
CTTTCCATCGGAGCGTTGTCGTAACAGTTACCTTTACGCGACATTGATGTTTTCAGACCAAACTGCTCCT
GTATGACCCGGTAATCGTATGCGCAGTACTGTGAACCTCGATCAGAGTGGTGGATTAGCCCGGCAGGT
GGGCGCTGGCTCCTGAGCGCCATAAACAGGGCTTTACCTGTCAGCTCTTTTGTCATGCGCTCTCCCATG
GCGTAGCCGACAATTTCGCACGTATAAACATCTTTGATGCCAGCGAGGTACAACCATCCCTCCTGTGTG
GCAACATACGTCAGGTCCGCCACCCAGACCTGATTTGGTGCTGTAGGAGCGAACGTCTGGTTCAGCAG
ATTTGGCGCAACTGGCAGATTGTGGTTCGAGTTCGTAGTCGCTCTGAACTTGCGTTTCTGCTTACAGCG
TAGCCTTAGCTCCTTACGAAGACGTGCCAGTCGGTCACGACCAACGATGATGCCATTCTCTGCCAGCTC
CGTCTGGAGCCGCCGGGTTCCATATGTTTCGCGAGTGCGGATATGTGCCACCTTAATCTCCAGTTTTAG
CCGCTCATCACTTTGTTTTCTGTCTGAGGGTTCATGCTGTACCCAGTTGTAATAACCGCTCCTGGATACA
CCAAATACCTGACACATCGCTTCAATGGGAAATTGTTGTCGCCATTGTTCGATTAACGCGTATTTTTCAG
CGACTCCTGTGCAAAATACGCTGTTGCTTTTTTTAATATATCTCGCTCAAGGCGAGCTTCATTTAACGCCT
TACGCAGTTGCAGAATTTCAGATTCCAGTTCAGCCACCGTGCGGGAACCAGGAGTACCGAGCCCTTTTC
TGGCGGCGGTAACCCATTGTCCTAAAGTGCCTTCAGGAAGAGATAATCGGGAAGCGCCTTCACTGATC
GAAAGTTGATTTTCAAGAACCGTTCTGACAGCTTCGGCTTTGAACTCTTTAGAGTAACGTTGGGTTTTTC
TGCTCATTATTAGCTCCTTCTGATGCCATTCTATTTCAGGAAGGAGTGTCCGTTAAACTCAGGCTACCTC
ACAGAGGTGATATGTTCACCTCAGAACAACACAGGTGCTCCAATGAAAAAAAGGAATTTCAGCGCAGA
GTTTATTTACGGGGTGACGCTTTCATTAACCTGTACACGAACTTCTATTCCGGCATCATGACAGGCCTGC
AGCTACTGCGCCATTTCCAGCGGATCGCCCTCCCGGCGTACCACCCCACCTTCTTTATTCCATAACTGCA
GAGCATTTTTCTGATTGTTCCATCTGATTTCGGGGGGTATTCCGGGCAAGGTTGTTCAGTAAGGCATTC
CTTCGTAAAATCAGCATGTTTTTTGCACGACGGAAACCGGCAGCCATGAAAATCTGTCTTTATCATACTC
TGAATCCTGACACGATCCCGGGTTATAAAAAATTTGCACAGGCTATTGCCACCGATAACTTTGTTCAGG
CTGATGTTCGTAAAATTGACACCAATCTTTACCGCGCCCGGCTCAGTATCCGGGACCGATTACTGTTTTC
GCTGTATCGCTACCACGGTGAAACTATCTGCCTTGTTCTGGAATATATTCGCAATCATGCCTATAACACT
TCACGTTTTCTTCGCAGAAACGTGGTGATTGATGAAGGGCGCTTACAACAACAACCAGTGCCGGATCCT
GTTGATATCGCGACTGAAGCGCTGACGTATATCAATCCGTCTCATGGGCGTTTCCACCGCCTGGATAAA
ATGCTGTCCTTTGATGATGATCAGCAGGCACTGTACGAACACCCGCTACCGCTGGTGATTGTTGGCTCG
GCGGGAAGTGGTAAGACGGCGCTGGTACTGGAAAAAATGAAGCAGGCTGCTGGTGATATACTGTATC
TCAGCCTTTCCTCATTTCTGGTGGAAAAAGCGCGTACTCTCTATGACGCTTCAGGGGAAGGGAGTGAG
GTACAGAATATTGACTTTCTTTCGCTGACGGAATTTCTGGAAACTCTGCGCATTCCGGAAGGCAGGGAG
GTGACTTTCAGTGCGTTCAGCGACTGGCTGCCCCGCAACAGGGCTATCGCCGCACTGGGGGCTGCCCA
TACCCTGTATGAGGAGTTCAGGGGCGTGATCGGTGCGGTTGCCTCCGGGAATGGGCCGCTGTCCCGG
GAGGCGTATCTGTCACTGGGGATACGGCAGTCGCTCTATGGCATGGAGGACCGCCCGACGGTCTATGT
CCTGTTTGAACGTTATATCGCATGGCTGAAACAGTCGCATCAGTATGACAGTAATCTGCTCAGTCATCA
GTATCTTTCGTTGGCAACTCCCCGTTATGATGTCATTTTTGTTGATGAAGTTCAGGATATGACGCCTGTT
CAACTGCAACTGGTACTGAAAACGCTACGTCATCCCGGTCAGTTTTTACTCTGTGGTGATGCCAATCAG
ATAGTTCATCCGAGCTTTTTCTCATGGAGTTCGCTCAAAAGTCTCTTTTTCCGGCAGCAACAGGGAAACG
ACACAACCGTTAATATCCTGCAGGCGAATTACCGTAACGGGCACCACGTCACTGCACTGGCTAACCGGT
TGCTGAGACTCAAACAGGTCCGCTTTAGTGCTATTGATCGGGAAAGTCACCATTTTGTCAGAAGCTGCG
GTCAGGCAGAAGGGACCATCCGGTTGCTGGACGACAGGGAAGAGACAAAGCAGGAACTGAATGCCA
AAACCTCACTTTCGAATCGTGTAGCGGTCATTGTGATGCATCCGGAACAGAAAGCACAGGCGCGATGC
TGGTTCAGTACGCCGCTGGTCTTTTCGGTTCAGGAGGTTAAGGGGCTGGAATACGAAACGGTGATCCT
TTACAATATAGTAAGCGCTGCCCGGCAGGCATTTGATGATATTTGCGAGGGTCTTACACCGGCTGATCT
TGAGGGCGAGGCCCGTTATTCCCGTCCGCGGGACAGGCAGGATCGTTCAGCGGAAATCTATAAATTTT
TTACCAACGCGCTGTATGTTGCGCTCACCCGTGCTACACACAATGTGTATCTTGTTGAACAGCAGGTGG
AGCATCCTCTGTGGAGTCTGCTTGCGCTCACGCATCAGGAAGAGCCTCTGAATCTGCAGGAAGAGATTT
CCAGCCGTGATGAGTGGCAGAAAACCGCACATCTGCTTGAAAAACAGGGGAAGCAGGAGCAGGCGGA
CACCATCCGTAGCCGGATCCTGCAGACATCTGAGATGCCATGGCAGATTATCACCGCAGAGGATGCAC
GACAGTGGAAACAGCATATCCTCGCAGGTACAGCCGATAAAACCATACAACTGCAGGCGCTGGAGTAC
AGTCTGATTTATTCCCTGTTCCCGCTGTATAATGCACTGTACCGGGAAGATTTTAAGCCCACAAGGCAGC
CCAGAACGAAGACTCTGCAGTTACTGGAGCTGAAGTATTTCCGGCCATACTCAATGAATAACCCGGTTG
CCGTGTTGCGGGATATTGAACGTTATGGTGTTGATCACCGTTCGCCGTTTAATCTTACTCCGCTGATGTC
AGCAGCCCGGGCAGGTAATATTGCACTGGTTCAGTTACTGCTGGAGCGTGGTGCAGATCCCTTGCTGA
CCGGTAATGACGGGCTGGCGGCTTATCATCAAGTTCTGTCAGCGGCGGTCAGTACGCCCCGTTATGCG
CAACAGAAAAGTGCGCAGTTGTATACCCTGCTGAAACCTGAAAGTCTGTCACTGCAGGTGGAAGGACG
CCTCATCAAACTGGACAACCGGCAGATGGCAATGTTTCTGGTGATCCTGATGCAGGCGCTGTTCCACAC
GCATCTGGGAAGTGCTCTGTTTTTTTCGGAAGCCTTCAGTGCGGCACGACTGGCAGAGTGTGTGGTTCA
TCTTCCTGAAGCGTTGCTGCCGGAACGCCGCAAACGAAGGAGTTATATCAGCAGCCAGCTCTCTCAACA
TGAAGTTAACAGCAAAAATCCTTATGGAAAAAAGCTTTTTCTGAGGCTGAACCACGGACAGTATATTCT
TAACCCGGGGCTTAAAATAAGGCAGGGTGATGTGTGGCGCGCGGTTTATGAGTTGCAGTCACCGGAA
GATCTGGGGCATGATTTGCAGACTTATCTGCAGGATATGTCACCGGAATTAGTGGATATGCTGGGAGG
GAAGAAGGGTTTTTACGAACGCAGTGAAAAGTCTGTCGGATACTGGGTGGGGGGGATCCGTCGTGCC
GCTCAAAAAGCGTGAGGTTCCGTGCTGACAGGATGTAACCGAACTTCATCTGTACGTAAGTACGTGAT
GTCTCCCGCCCACTTCTGGTTCGGACCACTGGCGTAAAAATCCTGCTCCAGCAGATTTTCTGACACGGG
CAGGCCATGTGCGCGGTAACTGACCGGGCTGAACTTCCGGGAGGCCTTTGCCCTCAGCCCCTGACGGC
GCAGGCTTGCCGCCACGGTTTTTACGTTAAAGTGGTAACCCTGAGCACGCAGTTCATCCGTCAGGCGTG
GTGCACCGCAACGCTGTTTTGACCGGGTAAAGAACGCTGTCGCAGTGCTGACGGAACTGTTGACGAGG
GCTTATCCCTGTCCGCCGCTGACACCACGTTCAACAGTTTGTGGACTGTTCGCTCGCTTCCGGAAATCAG
GGCTTTCATGGCCTTTGCCTGCAGGTATGTCGGAGCAGGAACTTGATGCCCTTCTTTACGGAAGTGCCT
CCACAGTACCTGTCGTACTTACTGAAAGCACCGTTATGCCCAAACTCCCGGTGGTAAAAAAACGTCCCC
GGCGGCCTTAATGCCAGTCAGTTAAGCAACTGACTGGCTCTTTTTCGGGGCTGTGGGGTATTTCCAGGG
CCTCTCCTTTACCACTCTCGGGAAGGCCCTTTCCCTTCTCGTCGGTAATTTCACAAGTTGTCCCATACTTG
CAAGATCGCGCATCAGCTCCGGTATACGTCCCGGTGAAGCGCCCTGCAATGTCATCAGCATTCTCATCA
CCATTCCGCATGATTCTGAGAAACTCAGTTGATTCGGCCAGTAACCTTTCAGATGTTCCGCCATTTTAAT
CATCTGATATCTCACCAGATTATAAGCCAGTAAGACACCCCACAGCTCTTGCTCCACAAGCTCCGGCTTT
TTACTTCTCAGCGTCAGCCTGCTCAGTTGCATCGTCTGTTTTATCTCCCTGTATCCCAGTTCGATTTCCCA
GCGATGACTGTACAGATCCGCCATTTCTCCTCCGGGGAAGCGCATGGCGTCCGTCATCGACGTCAGCA
GATGGCAGACTTTTCCTTTGCGCGTCACGGTCAGCAGGCGGGCTGTCACTTCATTTCCCAGTCCCGGCC
ACTTTTTTCGTGCCTGCGGGCTGGTTTTCAGCTTCACCAGATGATCGCCTTTACCCAGTTTTCTGAGCTCT
TCATATTGCGCTCCCTTTCTGAGAGGTATCATCCAGTGGCGGTGTTCTCCCGCCAGGCTCCAGGCATTTA
ACAGTCCCAGTGAGTAATAACCTTTATCCATTAACGTCAGAGTGTTATCGCCGGTTTGTTCTATAAGTTG
CTCAGCAAGCTCATTTTCGCTGTTCTTCATCGTGCCGAAGGCTGCAGCCGTCAGCAGATGGCTGGTCAG
TTCCATCTGGCAGACCATTTTGACCTGCGGGTAGAGCGCCGGGTTCCCGGCATGTGTCTGGCGGGGGA
AGGCTGCATCGTTCTCTGGTGTATCCGGTGTGCGCCAGAACACACCATCGATGGCCAGCAGGGTCAGG
CCGCACCAGTGCGGATGCGGCGTGGCGTTATGCCAGAGCTGCGCTGTTTTCGTGAACACGCGGCGGAC
AGCCTCACTTCCCAGGCGCTGGCGGGCCTGAATAACGGCACTGGGGGCAACGAAGGGGCGATTGCCC
GGCAGCATGATGTCCAGGCGATTCACAATCTGGTGAAGAGGTTCTTTACGCTCAAGCGCCATGCCAAC
AATACACCAGACCATCATTTCGAGGGGAAGACGGCGCTTGCGTAGCGTTACAGTACCTGATTCGGCAA
GGCAACGAGAGATGAGTTCGGGGTCGAGGTAATCCCCCAGAGAAGTCAGTGGGTTACGCAGAGAATC
GTAACGGGATACCAGATCAAGAGCCTGTCCAATGTGCATAAAAAAATCCGGAAACAAGTGAGCGTTTC
CGGATTCTTACACAGCCACTGGATCGGTCAACTGATCCTTAACTGATCGGCATTAGGCCGCCGGGGACG
TTTTTTTACCACCGGGAGTTTGGGCATAACGGTGCTTTCAGTAAGTACGACAGGTACTGTGGAGGCACT
TCCGTAAAGAAGGGCATCAAGTTCCTGCTCCGACATACCTGCGGGCAGAGGCCATGAAAGGCCAGCGT
TGCGGAAGCGCACGAACATACCACAGACAGTTGATTTTGGTACGCCCAGGCGACGTCCGGCCACAACC
CGTGGTAAATGTTCTTCGAAATGAAGGCGTAAAGCTTCAGTGATTCAGGTCCGGTATTCCATACGATAG
TGTCCACTTCAAAATGATGGACATTATTTTTGTGGAACCGGAGGTAACAAACCAGACGGTTTAAATGAG
CCGGTTACGTTTCAGGTGTCTCTGGCTGGAAATCCGTGCGTGATTCGCCTTGCTGCACAGGTATGCATG
TGGTTAAAATTTATCATACGCGATCGCGGCGGTTTTTCGGGTGGTTTGTTGCTATTTTTACCTGTCTGCT
GCCGTGATCGCACTGAACGCATTCTGGCGGTGCATACAATTAAGATATTACGTTAATATCGCGAGGGA
ATGGTCCCCATATTCTCATAGAAGAGCCTCTGGCTTATAGCATCAACAAATACGGCTATAAATATTTCCT
GTTTAATAACGAATAAAAATCATTTGTACGCATTTGTAATCGTCGGTCGATAATCTCTTTATCTTTTTTGG
TTGCAGCGTAATATTCAGCATAAATATTGCGAAAATCTATTGGGAATAAACTTTCACTTTCTTTATCATAC
AGCATATTACCTGTATTAAGATCGTAATGGATTATACTCAACTCATTCAATTTACATATCAACTGTAGAT
AAAGGCTCTCTATATTATCAGGGATATCAGCTGTATCAATGTCACTAAGGGGAGTCCCAGGCACGCGCA
GCATTCGGAGGTACACATCTCCGCCATACTGTATAACAACGGATGCTTCATCGCCATAAAAAGCATTAA
AAAGCTCAGATTCTTGCCAAGCCATTTCCAGAATCTCATTGTACTGGTTGCCAATAAGATCATACTTTTT
ATACAAAGCAGATGAATCATTCACATCTTCAAAGATTTCTGCTGTCGACCCCTGACCTATTAACTTACCG
AGAATGGGCTCCGTTACTATGCCAGCATGAGAATTATTATTCTCAAATGGAAAAGGTGTTTGAATAATG
GTAGATGTTATTTTCATGTCATACCTTTGTTACGATATTTAAAAATCACAAGAGATTTTCACACAAAATA
ACGTTACACGCTACCTTGTGTGGTCCAGATGTTCCTTCCACACGACCGTTGCGGCCTTTATACGGCTGCC
CTGCCAGGCTGCTGGCTGAATAACCAATCCGTTCGTCCGGTTCAATGGCGGCCATCAGGTTCTGCTCCG
CAATGTACTACGAATGCGCTTCCTGTCCTGGATGGCCAGCATGGTCCAGAACATCTCCATCATTTATCAG
CAATCTTCCTGCTCATTTCATCCCGCCATGCAGCAGCAGAGGATTCGACCATGTCATGGGGAAGCACAT
CACCGCATTCGAGTTGCTGACGGCCTGCCGCAACCTGGTCACGGAACCAAGAATTGTGCTGCCGTTCAA
CGGTCTGGCGCATAAAATCCCGGATTAACTGAGAGCCATTACGGTCCATGCTTTTTGCAGCGGCCATAA
AAGCATCCTTCAGTTCTGCGTCGATTCTTACACTCATATTAACCTGTGCCATTTGCCACCTCGTCATTACC
ACGTATATACACAGTATATAACTGTTCAGATATCGTCACCAGGATATGCCGCGCCAGCGGCATGTAAGG
CGGCACTCCGTTGTTGCATATGGCAGAGGAGTAAAACCGCCGAAGCCCGGCGTAAGCCGGTACGTATT
GACAGGTTTCACCTGTCCTATCACTGGCGCTGACAGTTTTATGCGATGTGATTATGAATGGTGCAGTTG
TGAGCCGTTTTCAGACAATCGCAGGGCCAGTACCTCGTCAGTCAGCTGACGGTAAATCTGCTGTTCAGT
CTCACGCATCACCTGTGCACCGGCTTCCCTCTCCGCATCCGCATCACCGCTCAGACCTGATGCTTTCAGC
CGGTCAGCCACCCTCTGAGGGTACTCATTCTCCAGCATCTCATATTTCTGCTCTTCTGCCAGCGCCCAGC
GGTCAGCTTCCGTACGCTTCAGTACAGCATGCCATGGTCCCCAGAGGGAGAACCAGTCCGTAAATTCAT
TCTCTTCACGGCTTCTGACCGTGGCTTCGGCAGTGCGGAGGTCATTTGCTGTCACTCCCGACACGCCAT
AGAAACGCATTTCCTTCACGGCAGTGAAGAGCTGAAGTTTCTCTGCGAGCATGGTCTGGAAGGCCAGG
TAGACTTCTATCTCATCCACAAAATGGAGAGTTCTGACTTTATCCCGGGCAATGTCCTCCAGAATTTCGA
GGCGGAACATTTCCCTGCCCAGGGAGAGCAGAGCGCCGGTATCATTATCGAAAAGGCCTTCTGATGCC
TGATGGACCAGGAGGGTTTTCCGGAGATTGTTCCATGTGAGCGCGACACGGTCCTCACAGCTCTCAGT
GGCATCAGCAGCAACAGCGAAAGACTGCTGTCGAAGCTCCGCAGAGGCACTGAGTTTTTCCAGCCATG
CAGCGACCTGTTCACGGAATCCGGAGGTATTGCGTGCAGAGACGGTATCGGAAAGGCGGTCAAGGAA
CGCGGAAAAGGTGTTGGCATGCTCTTCATGTTCAAAAGCATGCCATATCTGTGATACATCAGATTGTTT
GTTTTCCGGGAACCATGCTGTCACGGCATCAGCCAGGGGGCGATGGAGTGTATTCTGTTGTCCGTCACT
CATGGAGAAGTAAATCCGTGGGCCGTGGTAGTCCGGCGAAGAGGTTAATCTTTGCAGGGCTTGCAGA
GCATGGGATGATAATGGGTTATCACTAATATGTATTGAACATTCATTCCTCAGATTAAGAATGCTTTCCG
GGATATGACTTATCTGATTTCTATCAAAAAAATACTCTCTGACAACGGGATTATTTCCCTCAGAAAATGC
TGGTAGCTCAGTGAGGAAATTACGGGTCGCTCTGAGATTTTTAAGAGCCTGTGGGAGCGATGGTAAGG
AGACTATTTCGTTATAGCTGATATTCATAGTCAATAACGAATCGGGTAAAGCTGGCAGGTTTTCCAGCT
CATTGTGTTGTACCTGAAGTGACTCCAGCGCAGGAGGTAGCACGGGCAATTCTGATAATTTATTGCTGG
CGGAATATAATTTTTTTAGCGTAACAGGCAGTTCAGGTAGGTTAGTTAATTGATTATATGATACATTGA
GCAGCGTTATCTGAGCTGGTAAGTTGTCAGGTAGCGAGGACAGATTTAAACGGTCCAGTCGAAGTTCA
TCGGAATTATTGATAAGACATTCTTTAAGTCGGGAGACAGCCTCATCACGCTCTTCACCGGGGAGCGCT
TGTTTTTCCCATTTATCCCATGCTGAAAAGTAATCAGCATATGTACCGGAAATAGTGTTATAAAAAGAAT
TTTGGGGCAATGAAAAGTTATTATTTATCGGTAACATGATGGGCATCCAGTTAAAGCCGGTGGACAAAT
GGCTTTGTTAAAAGAAATAAGTAGGAGTTTTTATGAAGTTCATTCCATGAAATGAACTTCAGCACTTTTT
TATTATAATAAGGATAAACAAAATTGGCTGTAAAAAAACGATACTGTTTCAGTTAACCGTGAATGGCCT
GTTTGCACCGTATGTGACAGTAATACGCTGCTTCATGATAAGCCCTCAAAAACATACTTATGATATGTAT
ATGAGTATATATTAATGGTACAAACCATCAATATCCATAAAAAATACACCATTAATTCGCTATAAGTTTT
TAATACAATTTTTTCTGCTGCGATTTTTTTATACGATAATTAGCATACTTATAACACCGTAAGTAAATTAC
ATGAAAGCAGGCACAGCGTGATCCGATTTTTCTGCGTATAACGATTTTCCGGAAGTGTAATACGGTTGC
GCCCCGGGCCGGGCCCCCTTTTCTGTTGTTGTGTACGGGCCCACAGAGAATGGTACCGGAGGCGGGCT
CAGAAGTACTGAGTCGGGTGATCGCGTCAGGCCGCGAAGTACTGACACGGGTATTGGTGACTGGTGA
TGAAGTACTGCCCCGGGTATGATTTCAGGCTGATTCAGGTTCTGATTCTCTGTCAGTCCGCGTGGTTGCT
GGCCTGGCTGATTTCCTGTTGTCAGGCGTTCAACAACCGACTGCAGGTTATAGCCCGGTTCACGAACAC
CTGACGGCAGGTATCACTCACCCGGTGAAGGTAAATCCCTCAAGAGGCAGCAACTCTGCCAGTCGCTC
CTCCGGCCATTCCGGCAGACGCGTCAGGACGTCCGTCAGCCAGGCATGTGGCTCCAGACCATTACGTTT
TGCTGTTTACAGAAGGCTCATTATCTGTGCCGCCCGGTTTCCGGCCATCAGTGAACCGGCGAACAGCCA
CGCTTTCCGTCCCATAACAACCGGGCGAATGGCACGCTCGCAGATATTGTTGTCCAGGGGCACCGCTCC
GTCACCCAGGAAGCAGCTCAGTTCATCGCGCCGGTTCAGAATATAGTTAATCGCCTTATTTAATGGCCC
GTCGGGCGGACAGCACGGCTCCTGCTCTTCAAGCCAGGCAAACAGGTCATTCACTATCGGCCGGGAGT
ACCGTTATCGCCACTGCCGTATTTTTTCCACAGGGCGCTGTCAGCGCCAGTGATATAAGACGGTAATTC
GCCATTTGGATTGTCCGCTCCACCCAACATGTTGTTTCCTTAAGGTTCTCACACCAGAAAGGACATCAAC
ATGCTGAGCAGAGAGGACTTTTACATGATAAAGCAAATGCGCCAGCAGGGCGCGTACATCGTCGATAT
TGCGACTCAGATTGGTTGCTCTGAACGGACGGTCAGACGCTACCTCAAATACCCTGAACCGCCAGCCAG
AAAGACCCGCCACAAAATGGTTAAGCTGAAACCGTTTATGGATTACATCGACATGCGCCTGGCAGAGA
ATGTCTGGAATAGCGAGGTTATCTTCGCGGAGATTAAGGCAATGGGTTATACGGGCGGACGTTCCATG
CTGCGTTACTACATCCAGCCCAAACGTAAAATGCGTCCGTCAAAAAGAACAGTTCGCTTCGAAACTCAG
CCTGGATACCAGCTCCAGCATGACTGGGGCGAAGTTGAGGTGGAGGTTGCCGGGCAACGGTGCAAAG
TTAACTTTGCGGTTAATACGCTGGGGTTCTCCCGCAGCTTCCATGTCTTCGCCGCACCAAAACAGGATGC
TGAGCATACCTACGAATCACTGGTTCGCGCCTTCCGCTACTTCGGTGGTTGTGTGAAAACGGTGCTGGT
TGATAACCAGAAGGCTGCGGTGCTGAAGAATAACAACGGGAAAGTCGTGTTCAACTCCGGATTCCTGT
TGCTGGCTGACCACTATAACTTCCTGCCACGGGCATGCCGTCCACGCAGGGCCAGAACAAAAGGTAAG
GTTGAGCGGATGGTGAAATACCTCAAGGAGAACTTCTTCGTCCGGTACCGCAGGTTCGACAGCTTCACT
CATGTCAATCAACAACTGGAGCAATGGATAGCCGATGTGGCTGACAAACGGGAGCTTCGCCAGTTCAA
AGAAACGCCGGAACAGCGCTTCGCGCTGGAGCAGGAACATCTGCAGCCGTTACCGGATACAGACTTCG
ATACCAGTTACTTCGATATCCGCCATGTGTCCTGGGACAGCTATATCAAGGTTGGTGGTAATCGTTACA
GCGTTCCCGAAGCGCTGTGTGGTCAGCCGGTATCGATACGAATATCGCTGGATGACGAGTTGCGGATC
TACAGTAATGAGAAACTGGTGGCCTCACATCGCCTCTGTTCAGCATCGTCTGGCTGGCAGACAGTGCCG
GAGCATCACGCCCCGCTCTGGCAGCAGGTCAGTCAGGTGGAACATCGACCACTGAGTGCCTATGAGGA
GCTGTTGTGATGCATGAACTGGAAGTCCTGCTGAGTCGCCTGAAAATGGAGCATCTGAGTTATCACGTT
GAAAGCCTGCTGGAACAGGCAGCTAAAAAAGAGCTGAACTACCGGGAGTTCCTGTGCATGGCGCTAC
AGCAGGAATGGAACGGCAGGCATCAGCGCGGTATGGAGTCCAGGCTGAAGCAGGCTCGTCTGCCGTG
GGTCAAAACGCTGGAGCAGTTCGACTTTACCTTCCAGCCGGGCATCGACCGTAAAGTTGTCCGGGAAC
TGGCTGGTCTGGCGTTCGTGGAGCGCAGCGAAAACGTGATCCTGCTGGGCCCTCCTGGTGTCGGAAAA
ACTCATCTGGCCATAGCTCTTGGCGTGAAAGCGGTGGATGCGGGACATCGGGTACTGTTTATGCCACT
GGACAGACTGATCGCGACACTGATGAAAGCGAAACAGGAAAACCGGCTGGAGCGTCAGCTACAGCAA
CTGAGTTATGCCCGGGTGTTGATCCTGGATGAAATAGGCTATCTGCCGATGAACAGAGAGGAAGCCAG
TCTGTTCTTCCGGCTACTGAACCGTCGATATGAAAAAGCGAGCATCATACTGACGTCAAACAAAGGGTT
CGCAGACTGGGGAGAAATGTTCGGAGATCACGTACTGGCAACAGCGATCCTGGATCGGCTGCTACATC
ACTCAACCACGCTGAATATCAAAGGAGAGAGTTACCGGTTAAAAGAGAAACGTAAAGCTGGAGTGCTG
ACCAAAAACACAACGCCAATCAGTGATGATGAAATGGTGGAAAGCGGACAGCATCAGTAACGAAAGT
ATTTAGCGGGCATGAAAATGGCAAATAACGGTCAAACATCGTGGCGTTGACAGCGCTCACGGATGAGT
TTCTCAATGCGGTAAAGACCCGCGATTTTTCTCAGCTCCACGCCTGCACGGGGCTCCCTGCTGGCCTTAT
ACAGATTTGCGAAGCCGCGCCCTGCGTGACTCCAGCAACAGGCTGATGTGATGCAGGGGTGATTATCC
GCCAGAGATTTGTACACGCTGTATCCGTCACTCACCAGCGTACCGCCACACCAGCACTGCAGCCAGGTC
TCAGGGTATCTGAGGGCCCGTCCGGTCTGCGAGTCAAAACAGACGACCGGCGGACCGCTTCTTTCACC
GCTGACGTAAGCACACAGCCAGCCGGAGCAGGATTTACCGCCTTTCCGGGTATCAAGGAGCCTCAGGG
ACGTTTCATCTGCATGAATAACATCCCGTGTCAGAAGCTCATGGCGTAGCAGTTTCGCCAGCGGGGCCA
GTGCAGCTCCGGCAACACCGACCATATCCGCCATTGTACTGACGGGGAGCTCCACACCCATTCGGGAG
AAAATGTGCTGCTGGCGATACAGGGGCAGATGGTCGGTATATTTGCTGATGACAACCTGGGTAATGGC
TCCAACTTATTGATAGTGTTTTATGTTCAGATAATGCCCGATGACTTTGTCATGCAGCTCCACCGATTTTG
AGAACGACAGCGACTTCCGTCCCAGCCGTGCCAGGTGCTGCCTCAGATTCAGGTTATGCCGCTCAATTC
GCTGCGTATATCGCTTGCTGATTACGTGCAGCTTTCCCTTCAGGCGGGATTCATACAGCGGCCAGCCAT
CCGTCATCCATATCACCACGTCAAAGGGTGACAGCAGGCTCATAAGACGCCCCAGCGTCGCCATAGTG
CGTTCACCGAATACGTGCGCAACAACCGTCTTCCGGAGACTGTCATACGCGTAAAACAGCCAGCGCTG
GCGCGATTTAGCCCCGACATAGCCCCACTGTTCGTCCATTTCCGCGCAGACGATGACGTCACTGCCCGG
CTGTATGCGCGAGGTTACCGACTGCGGCCTGAGTTTTTTAAGTGACGTAAAATCGTGTTGAGGCCAACG
CCCATAATGCGTGCAGTTGCCCGGCATCCAACGCCATTCATGGCCATATCAATGATTTTCTGGTGCGTAC
CGGGTTGAGAAGCGGTGTAAGTGAACTGCAGTTGCCATGTTTTACGGCAGTGAGAGCAGAGATAGCG
CTGATGTCCGGCAGTGCTTTTGCCGTTACGCACCACCCCGTCAGTAGCTGAACAGGAGGGACAGCTGA
TAGAAACAGAAGCCACTGGAGCACCTCAAAAACACCATCATACACTAAATCAGTAAGTTGGCAGCATC
ACCCCCGATTAGTTGTTAATCATATTTAATCATAGTGATTACTTTCAGACGGAACAAGGCAAATAATGCG
AGTCAGCGCATATAAGGCGGCATACGGTGGTTACATGTAGCACAGGTGTAAAACCGCCGAAGCCCGG
CGCAAGCCGGTACGGTCGTCCGAATATCACCAGTCCCCCTCCGGCTCTGCGAGGCCATGCGGCCTGTCA
GCGATGAAAATGCCTCTGTGCGCGGTCGGAGTGGTCCCGACAAGGGTTTACCCGCAGTCGGGGCGTAT
CTCCGCGTTAGCGGGCCGTGAGGGCCGCTTACGAGCGTGTATGAGAGACGTACAGTAGTTTTGTCCGC
TCACACCAGTACAAACCAGTCGAAATACCGGTAACGACGATTACTCTTGTCTCTGATGCCGGTGGACTA
CGAGATTTTATTTACTGGCGTCCTGATGCGGTCAGGACTGGTATGGAACCTGTTTATGTGATACTTAAT
GCTCTTCTGGATTCCGGGCGATTTACGCGTAAACTTATTCTGTTAGGGCTGTCAGGCTCATTTTCCTATA
TCTTCGGCTCAATTGTTGCGACACTGGGAATGGGACTTGTTGTCGATTATCTGGGATGGGGGGCCACAT
TTATCGTACTGATACTGTCTGCTGTTTTTGCCATCATTTTCACACTGATGAGTCGTGAACGCTCACTGGA
GTTCGAAAAAGAGTAATTTTTTCAGGAAAAACATAAAATTACTGACTAAATATTTATTTTTTGAATCGCA
GGGCATTCCGGATAAGGTTGATATCGTGTCACGGCTGAAGACCTATGGATACAGTATTTCTGGTGTGG
AGACGGATGACGGATATAAGGCGCTTGTCCGCGCTTTTCAGCTTCATTTCCGGCAGAAAAATTATGACG
GCATAATGGATGCTGAAACCGCTGCCATCCTGTATGCATTGCTGGAGAAGTATTTCCCGGGTAAATAAG
GAGTGAGTCGTCAGAGCTGCGATATATCCCCTTCCTCCATGGGCTGTCTCTTATACACAAATCCCCACCC
GGGGATTTGTGATGTCAGATGTCCCCTGTAACACTATGTTTTCATTGCGCTCTATGAAGCTGAGTGAGC
GTAACGACTGGAGCTGCTTCTGCGGTGCTCCGGTGGCGAACGTGAAGTCATACTCTTCGAACATTTTCA
CCGCCGGGAAGGCTACCATTCGGGTATACATCGCCTGTTTACGTTGATGACGTGCCAGTTTTTCTTCATG
AAGCATGATGCTTCAAGAAGGGCACTGTTGCAAAGTTATCGATGAGGTAGCTGACCATCACTGCTGTC
GGAATCGATCTTGCCCTTCTGTTTCCAGTCGCCTCAGCAAACTATTTTTCAGTTACCACCTGTCCTGCGGT
AAGCGTAAACCGACCACCGTATGTAGCCATTAGACAAGAATTGGTAACTTAGACGCCCATCTGACATAG
ACGAACACCTAAGTATGGAATTACAGGACTGGCGAAAAGAACCTCGTAAAAAGTATTCGAATGAATTC
AAACTTCGGGTGATGTCTTCAATCGACAGAAGTTTCTGGTAATCTACTGGTATTTAAGGTTTTCCATGGC
TACCGTTACTGTTCATTGTCCGCGCTGTCATTCCGATGAAGTTTATCGACATGGCCGTAGTTGTTCCCGG
CATGAGCGTTTCCGTTGTCGCTCATGTAAACGTGTTTTCCAGCTCACTTACTCTTATGAAGCCAGAAAAC
CCGGCGTTAAGGAGCAGATCGTTGAAATGGCACATAACGGCGCAGGGGGCCGTGATACCGCCAGGAC
ACTGAAAATCGGCATCAATACCGTCATACGGACATTAAAAAGCTCCCGCCCCGGCGGATAACATCTGCG
CCAGTCGCTCACGCTGATGTTGCCCTTATCTGTGAACTTGATGAGCAGTGGCGCTTCGTTGGCAGCAAG
GCTGCAACAACACTGGCTCTGGTATGCGTACAACACTAAAACTGGCGGTGTGTTGGCTTACACTTTCGG
GCCACGGAATGATGAGACCTGTCGGGAACTGTTAGCCCTGTTTACTCCGTTCTGTATATATTAGGCCGT
TGGTGGTAAGTGGATCACTTTTTAGACTCGAATAGCTGCAGTTCACAAAAACAGGAATCCGGCTCCGTT
TTTTGTTGTCATATCAGAGGGATATTTGTTATGAAGAAAATATAGTGAGTATATATACTGTCAGGATAC
AATATGATTAATGGGGTGTCGTTACAGGGAACTGCTGGATATGAGGCACATACAGAGGAGGGAAATG
TAAATGTTAAAAAATTGCTGGAATCTTTAAACTCTAAAAGTCTTGGTGATATGGATAAAGATTCTGAATT
GGCAGCGACTTTACAGAAAATGATAAATCCGTCTGGTGGGGATGGTAACTGTTCAGGGTGTGCGCTTC
ATGCTTGTATGGCTATGTTAGGGTATGGAGTAAGAGAGGCTCCCGTACCTAATGAAATATCAGAGTAT
ATGACCGGATTTTTTCATAGGCATTTAGAGCAAATTGATAGTGAAGGCATAGTGTCACATCCTAATGAA
ACTTATAGTAAATTTCGTGAGCGTATTGCAGAAAATATTTTACAAAACACTTCAAAAGGAAGTGTAGTA
ATGATTTCTATTGAACAGGCCACTCATTGGATAGCTGGATTTAACGATGGTGAAAAAATTATGTTTTTG
GATGTTCAAACAGGAAAGGGATTTAATCTTTATGATCCTGTAGAAAAATCGCCAGATGCATTTGTAGAT
GAAAATAGTTCGGTTCAGGTAATTCATGTATCTGATCAGGAATTTGATCACTATGCTAACAGTAGTAGT
TGGAAGAGTAAGAGGCTTTGCTGATGCTGAGAGCCTGTACATAGATTTGAGCCGGCCCCTTGAAACGC
CAGACAGTGGATGTATCTTAAAATAAGAGATAGGCTTGGATATATGTCTAACACTAGTTCTAATTTTGA
GATGACTGGAACTTTGCTGGGGCTGGAGCTTCGTAAACGTAAAACTCCTCAGGAAAAAATTGCCATTAT
TCAGCAGACAATGGAGCCTGGCATGACGGTGTCCCATGTGGCGCGTTTGCATGGTATTCAGCCCAGCC
TGTTGCTTAAGTGGAAAAAATAATATCTGGAAGGAAGTCTCACTGCTGTTGCTGCCGGAGAGGATGTC
GTTCCTGCCTCAGAACTGGCTGCCGCTATAAAACAAATAAAAGAGTTACAGCGCTTGCTGGGTAGACTG
GCCCCCTGAATCTCCAGACAACCAGTATCACTTAAATAAGTGATAGTCTTAATACTAGTTTTTAGACTAG
TCATTGGAGTACAGATGATTGATGTCTTAGGGCCGGAGAAACGCAGACGGCGTACCACACAGGAAAA
GATCGCAATTGTTCAGCAGAGCTTTGAACCGGGGATGACGGTCTCCCTCGTTGCCCGGCAACATGGTGT
AGCAGCCAGCCAGTTATTTCTCTGGCGTAAGCAATACCAGGAAGGAAGTCTTACTGCTGTCGCCGCCG
GAGAACAGGTTGTTCCTGCCTCTGAACTTGCTGCCGCCATGAAGCAGATTAAAGAACTCCAGCGCCTGC
TCGGCAAGAAAACGATGGAAAATGAACTCCTCAAAGAAGCCGTTGAATATGGACGGGCAAAAAAGTG
GATAGCGCACGCGCCCTTATTGCCCGGGGATGGGGAGTAAGCTTAGTCAGCCGTTGTCTCCGGGTGTC
GCGTGCGCAGTTGCACGTCATTCTCAGACGAACCGATGACTGGATGGATGGCCGCCGCAGTCGTCACA
CTGATGATACGGATGTGCTTCTCCGTATACACCATGTTATCGGAGAGCTGCCCACGTATGGTTATCGTC
GGGTATGGGCGCTGCTTCGCAGACAGGCAGAACTTGATGGTATGCCTGCGATCAATGCCAAACGTGTT
TACCGGATCATGCGCCAGAATGCGCTGTTGCTTGAGCGAAAACCTGCTGTACCGCCATCGAAACGGGC
ACATACAGGCAGAGTGGCCGTGAAAGAAAGCAATCAGCGATGGTGCTCTGACGGGTTCGAGTTCTGCT
GTGATAACGGAGAGAGACTGCGTGTCACGTTCGCGCTGGACTGCTGTGATCGTGAGGCACTGCACTGG
GCGGTGACTACCGGCGGCTTCAACAGTGAAACAGTACAGGACGTCATGCTGGGAGCGGTGGAACGCC
GCTTCGGCAACGATCTTCCGTCGTCTCCAGTGGAGTGGCTGACGGATAATGGTTCATGCTACCGGGCTA
ATGAAACACGCCAGTTCGCCCGGATGTTGGGACTTGAACCGAAGAACACGGCGGTGCGGAGTCCGGA
GAGTAACGGAATAGCAGAGAGCTTCGTGAAAACGATAAAGCGTGACTACATCAGTATCATGCCCAAAC
CAGACGGGTTAACGGCAGCAAAGAACCTTGCAGAGGCGTTCGAGCATTATAACGAATGGCATCCGCAT
AGTGCGCTGGGTTATCGCTCGCCACGGGAATATCTGCGGCAGCGGGCTTGTAATGGGTTAAGTGATAA
CAGATGTCTGGAAATATAGGGGCAAATCCAAATTAGTAGAACATATGTTTTTCGATAAACGCACCGATA
ACTTTTTGAGGTAGCCTGAGTTTAACGGACACTCCTTCCTGAAATAGAATGGCATCAGAAGGAGCCAAT
ATTGACGGGTAAAGCCCTGTTTATGGTGCTCAGGAGCCAGCGCCCACCTGCCGGGCTAATCCACCACTC
CGATCGAGGTTCACAGTACTGCGCATACGATTACCGGGTCATACAGGAGCAGTTTGGTCTGAAAACAT
CAATGTCGCGTAAAGGTAACTGTTACGACAACGCTCCGATGGAAAGCTTCTGGGGAACGCTGAAAAAT
GGTACAGGGACGGAGTAAACCGTAGTAGCGGTGAAGGACCGTAATGGGTCCGGAGCAAAGGGCTTG
CCCCATTGTGCTGATGGTGCGGCGGCAACCACGGGCGACAATGCCGATGGGAGGACTGCTGTAAAATC
GGCAAAGCCATTTCCTGTAAGTAAACGACAGGTATGGGAAGCTTATAAACGAGTAAAAGCCAACCGTG
GGGCGGCAGGCATTGACGGGCAGACACTGGCCGGGTTTGACGAGAATGTTACAGACAATCTCTACAA
ACTGTGGAACAGAATGGCTTCCGGCAGTTATATGCCGCAGGCGGTAAGGCGTGTGGACATTCCCAAAG
CTGACGGTGGTGTACGTCCGCTCGGTATTCCGGCCGTGTCGGACCGGATCGCCCAGATGGTCGTTAAG
CAGATACTCGAGCCGGTACTGGAACCCCTGTTCCATGCAGACTCGTATGGTTATCGACCGGGTAAATCC
GCACACCAGGCGATTGCGCAGGCGAGGAAACGCTGCTGGAAGTTTGACTGGGTGGTGGAAGTCGATA
TTAAAGGCTTCTTTGATGATATCGATCATGACCTGCTACTGAAGACAGTGCAGCATCATACTCAGGCAC
GCTGGGTGGTGATGTACATTGAGCGCAGGCTGAAGGCACCGGTGCAAATGCCGGATGGAGCAATGCT
GGCACGGGGTCGAGGAACGCCGCAGGGCGGCGTAATCTCCCCCCTGCTCTCAAATCTGTTCCTGCACT
ATGCCTTTGATATGTGGATGCAACGTCAGTTTCCCGGCGTTCCCTTCGAAAGGTACGCCGATGATGTTG
TATGCCACTGTCACAGCCAGTGGCAGGCTGACGCTCTGATAAGTGGACTGAGGCAGCGGCTTGCGCAA
TGCGGGCTGCAGCTGCATCCGCAAAAAACACGAATAGTTTACTGTAAGGATGCTGACCGACGGGGGG
ACTACCCGGAAACGAGCTTCGACTTTCTGGGCTATACATTCCGGCCGCGTCTGTCGATGAATCGCTGGG
GTAAGACCTTCGTTAACTTCAGTCCGGCGATGAGTGCCAGAGCAGGAAAAGCAATCCGTCAGGAGGTG
AGGAGAATCGCAGTGACAAGTCCTTGTACGAGCTGGCGCATATGTTCAATGCGAAAATCAGAGGCTGG
ATAAAGTACTACGGTGCTTTTTATAAATCAGCACTGTATCTGACGCTGAGGCAAATCGATCGCAAACTC
GTGTTATGGCTGCCCCGTAAACACAAGCGACTTAGGGGCCACCGACGACGGGCCAGCCACTGGCTGGC
CCGTGTTGCGCGGAGTGAAACGAGGTTGTTTGCACACTGGCCGTTACTATGGGGACAGGCCTCAATGA
GAAGAGCCGGATGAGCTGAGAGGTTCACGTCCGGTTCCGTGAGCACCAGAGGGGGAGGTTCCCTCGG
GTGACTCGACTTGTAGTACATTGTTCACGAATGAGTGATGCAACAAGGCTGAAGAACAGGTTATCGGT
GCGCTTCTCGGAAGTCGGGTTGGTCCTGAACGCAGGGAAGACGAATATCGCCTACATTGACACGTTTA
AAAGGAGAAACGTCGCTACGAGTTTCTCCTTTCTCGGATATGACTTCAAAGTGCGTACTCTGAAGAATT
TCAAAGGCGAATTGTACAGAAAATGTATGCCGGGTGCATCAAATGCAGCAATGTGCAAAATAACAGAA
ACAATCAAGAAGTGGCGTATACATCGCTCAACAGCTGAGAGTTTGCTGGATTTTGCGAGGCGCTACAA
TGCGATAGTGAGAGGCTGGATTGAGTACTATGGAAAGTTCTGGTCCAGAAACTTCAGCTATCGACTGT
GGAGTGCAATGCAGTCACGTTTGCTTAAGTGGATGCAGTCTAAATACAGACTTTCGAACCGGAAGGCT
CAGCGAAAGCTGGCGCTGGTAAGGAAACAGTATCCGAAGCTATTTGCCCACTGGTATTTACTACGTGC
ATCGAATGAGTGATCAAGAGCCGTATGACGCGAGAGTGTCACGTACGGTTCCGTGAGCAGCCGGAGA
GTGAAACTCTCTCCGGCGGGGTTCCGATAGCGGTGGCGAACATGCGGCGGTGTTGTACTCGCTGATCG
GCACATGCCGTCTGAACAATGTGGAGCCAGAAAAATGGCTGCGTTACGTCATTGAGCATATCCAGGAC
TGACCGGCAAACCGGGTACGCGATCTGTTGCCCTGGAAAGTTGATCTGAGCTCTTAGTAAATATCAATA
CGGTTCTGACGAGCCGCTTACGCAGATCATGCCATAATGAATAAACTTTAATCAGCGAAATTGACGAAG
AAGCTGGCAGCGCTCCAACATCAGTTAAGCCGCAAGGTTAAATTCAGCAACAACTGGCAGAAGCAGAA
ACACAAAATACAGCAACTGCATTCCCATATCGCAAACATCCGCAGAGACTACTTTCACAAAGTCACAGC
GACTGTCAGCAAAAAACACGTGATGATCGTCAATTACGTTATCACTGCCAACTGACTTCACCTTTTGGTT
CATATGAAGCAGCATCCAGCGGTGAATGTTCCCGGATATATTCGATAAGTACTTCAGCATCAATAAATC
CGGTATTTATGTATCCGGGTCTATTATCAATACGTGGATAGCCATCCCCACCAGTAGCATTAAAGCTAAA
TGTTGTCATACGATATTTTTTGTTAAGGTCAACCGATTTACCTTTTATTTTTAGCATTTCGATTTTTCCTTTT
TTGACTACAAAGCTGGTATTCAAAAATTGTGGGTAGGCTCCGGAATCCGGTTTCATCTGTGCCACTGTA
GCTAGGTAGTCAGCTACTTCTTTCCCCGTCAAATCAATACTTACTACCGTATTACCAAATGGCTGAACTTT
CAGTATATCTTTGTATGTGATGTTACCACTCTCTATGGAATCTCGAATTTCCCCACCACTGACAACAGCA
AAATCTGCGTCAATACGTTCTGTCAGAGCGCTCAGAATCAGATGGCCCATACTGGTCTGAACAAAACGA
ACTTTACTTCTGTCCCCTTCCAGACGTCCGTTCACAACCCCAACCTTCACATCCAGTTGTGCTTTGCCTTT
ATTCTGGAATGGTGTCAGTAAGGATAACATCTGTGGATCTTCAGTAATTTCGGGGGTGTAAAAAGAAA
ATTCCGTTTTTCCATCTTCTCTGGTAATTCTCATTTTGAGATTAACTGGGTGTAACTGATAATTAACCAGT
TTCATTGTACCGTTACAGAATTCAAAATCAGCCTGACCGACATATTTTCCCCACTCATGCGCTTGTACAAT
CCAGATGCCATTTTGTTTATCTGGTACACAGGTTGTCCCCGGAATATAATTCCACTGTTTTTTATTATCGC
TGGCCATGCAGACAGGAGCTTGTGAATGTCCTCCCACAATCATTGCTAATGAGCCAGTCGGCAAACTGC
GAGCCATTTCAACATCACCAGGAGCATTCGAACCTGACTCGCCATTGTCATAGTGACCCATATGTGTTG
CCGCAATAATAATATCTGGCTTTTCCTGCTGATTAAGTTCATCAATGACACTCCTCGCCTCAGCAGCAGG
TTGACGGAACTCAATATCAGTAAAATATTCAGAATTTCCTGTTTTCGCAGTATCATCTGTCGTCAAGCCA
ATGACTGCAATTTTCAAATTCTGACGGATGAAAATTTTCCATGGACTGAATAAACGCCTCCCTGTACTTT
TTTGATATATGTTAGCAGAAAGGAAAGGAAATGTGGCCCATTTTTCCTGCTGACGTAATATATTTAGAG
GATTATCAAATTCATGATTACCGACGGCCATTGCATCATAACCAATTAGATTCATTCCACGAATATCGGG
TTCAGCCTTCTGAAGGTCTGATTCAGGTACACCGGTGTTAATATCACCACCAGATAATAATAGAACACTT
CCGCCATTATTTATTACTTTCTGCCGGATATTGTCCACCAGAGTTTTTTGGGAGGATAACCCGTACTCAC
CATGATTGTTACGCCAAAAGTGTCCATGGTGGTCATTTGTATGCAGAACGGTTATTTTATATACTTTATC
TGTTTCATACGCTATAGCAGGATTTCCTGTCAGTAATGACAGGGTGAACATTATTAGCGTAATGTTTTTT
TTTAACGGTATCATTGTACCTGTATGAACGCATCCCCGTTTGCAAGGCTTCCGTTGTTCTGACATCGACG
AGTAGTTGCTGCTCTGTATCCGGCCTTATTGCAGTCAGAGCTGCGGGCCACTATAAGATTCGCTGACCT
GAGCCTTATTCCCTTCACGGACTTTATGTCTAGGACACAATTCAGGTTTATCAGGTCACGGTCTTACCTG
TCGTTGTCATCACGTCGATCACCATAGCCCCCTTTCAGCATTCACCCTTCTATAAGGTTTGATGTTTATCA
GTTTCAGCAATTAATATGGTCTGATACTGACGTGAACCGCCCCAAGTTTCCTGGAGAATGTTTTATCTGT
GAACTCAGGCTGCCAGATCATCGTTTCCGATAGAAGCATAATAAGCTTTTTCTGCTTCTGCCGGAGGGA
TGTGGCCCAGCCTTTCCAGCAATCGTCGATTATTATACCAGTCCACCCACGTGAGTGTGGCCAGTTCCAC
TTCTGCACGGTTTTTCCAGCTCTTACGGTGTATTACTTCCGCTTTGTAAAGACCATTGATGCTCTCCGCCA
TCGCGTTGTCATACGAGTCGCCTGTACTTCCTGTTGATGCCAGTAATCCGGCTTCCTTAAGCCGCTGTGT
GTAGGCCAGCGATACATACTGAGAACCTTTATCACTGTGATGGACCGTGCCGGACGGTCGACGGGCCC
ATAACGCCTGCTCCAGTGCATCCAGCACGAATGTCGTTTCCATGGACGATGAGACCCGCCACCCCACGA
TGTATCCGGCAAACACATCAATGATGAACGCCACATAGACGAAGCCCTGCCATGTGCTGACGTAAGTA
AAATCAGCCACCCACAGCTGGTCAGGTCGTTCTGCCACGAACTGACGGTTTACGCGGTCGCCTGCGGC
AACGGCTTTCCGGCTGATGGTCGTACGGACCTTITTACCCCGGAGAACACCGGCAAGTCCCATAACCGC
CATGAGACGTGCCACAGTGCATCTGGCCACTCTGATACCTTCCCGTAACAACTGACGCCAGACTTTACG
CACACCGTATACCTTGTGATTTTCATCGTATACGCGCTGTATCTCTTTCTTCAGCCAGTCATCGCGCTGCG
CACGGGCACTGCGTTTATCCGGATGATGTCGCTGTTGCTGACAGTGGTAATACGTTGACGGGGCAATA
TGCAGTTCGCTGCATAGCGGTCCGACCCCGTACTGCTCACGCAGCTTATCCAGCAGTGGCATCATTTTTT
CCAGAGGCGGTCAAACTCCGCCTTCGCAAAATAAGCGGAAGCCTGGCGAAGGATATCGTTACTGCGGC
GCAGTTCACGATTTTCACGCTCCAGCTCTTTCAGACGCTGACGTTCAGCGGTGGTGAGCCCTCCATCACC
GCCCCCGGTATCCCGCTCATGCTGGCGAACCCGGACACGCAGAGTCTCCGGCGTACAGCCAATCTTTGG
AGCAATGGAACAAATTGTCGCCCATTGTGAGTCATATTCGCCCTGACTTTCCAGAACCATACGGACTGC
CCGTTGACGGACTTCGGGGGAAAAACGAGTATTTTTAGTCATCCTGTTTACCTCTTTCTCAGGAAGTTTA
GTCTCCAGGATTCCCGGGGCAGTTCAGTTCAGGCTGTACGCTTCCCCATGTAATTTGCCTCGCCACTTCA
CCGGGCTCAGGACCCCGTAGGATCTTCACCGAAAAAAATGCATCACCAGTTGCTTTATTATTTGTTTTAG
GTAGACCTGTTACTAATTTATACCTACTCTGACCAAGTCTGGATAAGAAAGGCACACCTGCACATTTCTA
CTTGGAAATGTTTTCTTTTACCATTTTGCTCAAAAATTTCAACATCCAATATTCCTCGTACAGAACTACTC
AAGTCCTGAATAGAAAAAGTTCCAGCTGGTACAGTAGAGTCATATATTACTCGACCTTGTTGAGATACA
ACAACACGTGCATTAGTTTCTGCAATACCAGTAATCTGAGGCGCATAACCACGAATATTTGGCGGTAGC
ATTCGTTCATCGCTTTCTAATGCAATTCCTGTGTAACGCCAAGCAACAGCGCATTCTGGCGCATGATCTG
GTAAACACGTTTGGCATTGATCGCAGGCATACCATCATGTGCACAAAAACCCACCGAGCTTTTACACGG
TGGGTCCCAGAGAAATGCAGGACATCAACACGCCCTGCATTTTTATTATCAGAAGGTATATTTCACACC
CAAAATACCTTGGGTGTCTCTGTAACTGTTATTACCCAATTGCTGAGATACGTTTCCCCAGATGCTTAGG
TTATCTGCAAGACGCCCCCCCACACCAGTTTTGAGTTCAATCACATTACGGTTGCCTATCTGGTGATTTG
ACATGCCATTCATAATTACACCATATTGCTTTGAGCTCCATTTCCAATTCACTTCCAAAAATGGTTCGAAC
TTACGAAGAGTCTCCTTATCGATACAACTCTGACTAATTAAGGATGTACGCAATCCCAATTTTGCTTGAA
TATTATTGACCCCAGAGGATGTGACCATAGTGCCATTGTGTTCAATATAGTCGTTTGCTTTTACCCCATG
CCTAGTTATCTGGACTTGTGGATTGAGATACAATGCGTTGTCAATATTATTATGAGCAGTCCAGCGTAT
GGTTGGGTAGATATAGCCAGCTTCCAAAGCTCCTGTTATTCCTTTGCTGGAATATTTCTCACCAGTTAAC
CCATCTCCTTTGACTGTATTATTAAACCAACCGTACTGCAACCAAGCATCCATATATAGACCTGTTCGTTC
TTTTTCATTCTGAAACCAAGAACTGTACAAACCGGCAGTATAACCGCTCACAGTTCCTCGTGAACTATAA
CTAGTATACTTATTATGAGTTTTGCTATCCTGATATCCATAAGCTCCCATAATACCCACATGCAAGCCATC
ATGATCAGTGAAGTTTGTCTTCAAAATATCTCCACCAATCTGATAAACCATCCTGTTAGCTGTGGTTTTTA
ATTGTCTATCAGCTAAATTATGCTCATTATGTCCACCAACAGTACGAATCCACATGGTGGTTTCGCTGAG
CTGACCTGTAGTAGGATCAACAATAGTGCTACCGTGTCGGTCATAGAGAGAAGGACTAAACATCGTGT
TCATTGCTATCAGGTTAGCAATATAACTGCCAGCTTCAGGGCGCCATGTGTGAATACTACCTTCACCCAA
ATTAAGTGAAGATACAAGCTGTGAACTAGCTCGGCGACTACTCATTTGAGTAGACTCTTGATTGCTCAT
CTGTTTTGATTCTTGATTATCCATCTGACTAGTTAGATACCACTTATTGGTATTCAGTCCAGATACAGTGC
CCTGTTTCAGGCGATAGTCATAACTACCAGCAACAATACGGCCTTTCTGAATAAAAGCATTCTTATCAGA
AGAATCTGTCGAAATAATTTGAACACCTTCAAGAGTTTTATCTCCAGAGCCTCCCTCATTTGTCACTGCA
ACATAGGTAATACCAGACGCATGGCCTGCAATTTCCAGCCGGTCAGTTTTACTGTTGTCATTGCCGAGA
ACCGCGCCCAAAGATAATATTCCGCCCTTTCCATGCCAATTACCTTTCTGAACATAGGTCTGACCGACAT
TTGACGAACTATTATTGAGAATAACATGACCGTTTTTCTCCAGAGTCATGTTACCTGTGACAATGACGG
GGTCAGGCAAAAAAGGGGCATTAATATTATTGATTAGCACAGTCCCACTATTTAATAAAGTGTTAACAG
TTTGGTTCATGCCTGAAAAATTCAAGGTAGCACCTTTATTAACAATAACACCAGCGGTACGTGTCATAG
CTTCAACTGTCCCCATTTTTAGAATGCCTGCATTGATGTTGGTGAAACCAGAGTAGGTATTTTTTTCCGC
CAAAATGAGAGTTCCCTCCCCTGATTTTGTTAGGGATTTTCCGTCCCAGCTATTTATATTATGATGGCTG
AGATTATCTGCCAGAATAGTACTAACAGTAAGTTCAGCGTTTTCTTTCAGATTGAACTCTCCATGACTGT
CGCCATCTGTATCATTCCAGCTCAAACCGTAGATCAATTTATTCTGGTCACTGATATAGCTTTTTGTATAG
TCAATAGCACCAGTAGTGAGTAACGGATTGATGATAGATATATTAGAAACACTGAAACCTGTTATCCCT
TTATCTGCACTTAGCAGTTTATAGTCCTTCTGCGCCCAGTTATTTATAATAGCTTCCACTTTTACATTGTTG
CCAAATGACATATTTCCAGAAGCTTCCAGACTACTGATTCCAGCTAAATTTAAAGAACTGTTCTCACCAA
AAGAAACGTCACCTGAAACAGTGAATTTGTTACCTGATAGTGAAACAGTTGTACAATCACCAGCACATA
AATCACCTTCAATAGTTGGTGTGGTCCCAGTGTAATCATTGTTGATAATTTTCAGAATGGATGAATTGTT
AACCTGTACATCACCAGTAATGACAGAACCTTCATGGAGTATTAATGAATTGTTTCCACCAGTGATGTG
GATAGCATTACCTACTACATTTTGTGTTCCATCATCTTCTTTACCTGAAATTATATATCCGCTATTAGCAAT
GGTCATATTGCTACCATTTATAGCATCCCCCCAATGTCCACCATTACCGCCTAAAATAGCTCCATTGTTGA
TTATAGACAGGTTATCTCCTGTGATAGCATTACCACCGTAGCCATCATAACCATAACCGTAAGCAGCACC
TCCGTTGCCTCCAGCGAACGTGCCCTTATTGATTACAGAGAGGCTGCTTCCTGTGATGGCATCACCTCCA
TTACCGCCATCACCCTCACCATAATTGTTACCTCCATTACCACCGGAAATAGTCCCTTTGTTGATTATGGG
TAGATTGACTCCTGTGACGGCATCACCTCCATTCCCCCCATCACTATCACTATAACTATCGCCACCATGTC
CACCTGAAATAATTTCTCCATTTATTATAAACAGATTGTCTCCTGTAACAGCCTCACCACCATCACCGTTA
TGGTCAGCACCGCTACCGCCGCTACCACCAAGAATCATGCCTTGATTGATTATAGACAAGTCAGATCCG
GTAATAGAGTCACCACCATTACCGCCACAACTATTACCATCATTATTATCACCAGCACCACCATGACCTG
CGCTAATCATAATATCAGAGTTAATCCGTAGCTTAGTTCTAGATGCATGAGAGGGGTTGGAAGAAGTTA
TATACCACGCATCCATTCCATCTTCACCAGCTCCCAAGGGAGAAAGTCCATCAACAGGTGTTAATAATTT
TTCATAATTGTCCTCTGAAAAATGAAGTTCTTGAGTACCCGAAAGAGGAATAGCAAAAGCTATTGGCCC
CCCGAGCAACAGGGATGCACCAACAACAAAAGGAGAAAAAGACAATTTGCATGTTTTTTCCTTTACCGT
AGGTAATTCTCCGGCCCCCCCCTGTGAACATTGGGTCATATTACAAAAAAATTTGTGAATTTGATTCATG
CACTATATTATCAGTAAGTGGTTGATAAACCCCTAAAAAGATTCCGTTTTATCCGGAATAAAGGGACGA
TATATGCAAAAATATATTAAACAAAGCCTCAACCAAAACATGAGAGCTGTGTTATATATCAATGCAACG
GGAGAAATTTACCTGTGTCTTGCTTTGAAAAAAAGAGGGGGAAAGCTATATGTTCGATAATAAATTAAT
AATTTTACTATGATAGTCTGCTTCAGACCGCAACTTTTCAGTTCTTTCTGTACGCTTGCCCAAAGTTTAAC
CACTTTGTTTTTCTATACACCCAATGGTTCTATCTGCCCCTTGATGTTAATCCCCAAAGCCAATAACATGA
ATGTATTCATATCACAACTTTCCTACCAGACTCTCCAATGCAATAACTGCATAATTGTTTTTAGTGGAAAT
GTATCGTTTTCTTAAAGAGAAGAATAACATTCCATTTTATTATGTTCCCAATTTAATTATATTAATATATA
TTGGCATTAATAGGAAAATACATCAGGAGAAATCAAATGCAGACATCAAACATAACTAACCATGAAAG
AAATGACTCATCATGGATGTCAACTGTCAAATCAACTACAGAAGTAAGTTGGAATAAATTGAGTTTTTG
TGACGTTTTACTGAAAATAATAACTTTCGGCATATATTCTCCACACGAAACTCTAGCCGAAAAATATAGC
GAAAAAAAACTAATGGATAGTTTCTCACCATCACTATCACAAGATAAAATGGACGGAGAGTTTGCCCAC
GCAAACATAGATGGCATTTCAATACGGCTATGCCTTAACAAAGGCATATGTTCGGTTTTTTATCTTGATG
GAGATAAAATACAATCCACTCAACTTTCATCAAAAGAATATAATAACTTGTTAAGTTCACTCCCCCCCAA
ACAATTCAACTTAGGAAAAGTACATACTATTACAGCTCCGGTATCAGGGAATTTTAAAACTCACAAGCC
TGCACCAGAAGTTATTGAAACAGCCATTAATTGCTGTACATCGATAATACCAAACGATGATTGCTTCCCC
GTTAAAGACACTGATTTTAATAGTGTTTGGCATGATATTTACAGAGATATTAGAGCATCTAACAGCAAT
AGTACAAAAATATATTTCAACAATATTGAAATACCACTAAAATTAATTGCAGATCTAATTAATGAGTTAG
GAATTAATGAATTCATTGATAGCAAAAAAGAGCTTCAAATGCTAAGTTACAACCAAGTAAATAAAATAA
TCAATTCAAACTTCCCCCAACAAGACCTATGTTTTCAAACAGAAAAACTTTTGTTTACTTCACTCTTTCAA
GACCCTGCATTTATATCTGCACTAACATCTGCATTCTGGCAATCTCTTCACATCACGTCTTCCTCTGTGGA
ACACATATATGCCCAAATTATGTCCGAAAACATAGAAAACAGGCTTAATTTCATGCCTGAACAACGAGT
TATTAACAATTGTGGACATATTATAAAAATCAACGCAGTTGTCCCTAAAAACGACACTGCAATCTCTGCC
TCTGGGGGGAGGGCTTACGAAGTTAGCTCATCAATATTACCATCGCACATAACATGTAATGGTGTTGGT
ATTAATAAAATAGAAACAAGCTATCTCGTTCATGCTGGAACACTTCCAAGCAGTGAAGGGCTTCGCAAT
GCTATCCCTCCAGAAAGTCGTCAAGTATCATTTGCCATCATATCTCCTGATGTTTAAGCCAATATTATATT
GGCTGCCAGACTGTAAAAAAACGTTCTTCAGAACAGCTCTATAAACTATTAAACCAAAGCTAAAAGGAT
GGGGACCAATAGAATCTCAGCCCATCCTTTTTATATCATTATGAACCGCCCCGGAAATCCTGGAGACTA
AACTCCCTGAGAAAGAGGTAAACAGGATGACTAAAAATACACGTTTTTCCCCTGAAGTCCGTCAACGG
GCAGTTCGTATGGTTCTGGAAAGTCAGGGCGAATATGACTCACAATGGGCAGCGATTTGTTCCATTGCC
CCAAAGATTGGCTGTATACTGGAGACTCTGCGTGTCTGGGTACGCCAGCATGAGCGGGATACCGGGG
GCGGTGAGGTGGGCTCACCACCGCTGAACGTCAGCGTCTGAAAGAGCCGGAACGTGAAAATCGTGAA
CTGCGCCGCAGTAACAATATCCTTCGCCTGGCTTCCGCTTATTTTGCAAAGGCGGAGTTCGACCGCCTCT
GGAAAAAATAATGCCACTGCTGGATAAGCTGCGTGAGCAGTACGGGGTCGGATCGGTATGCAGTGAA
CTGCATATTGCCCCGTCAACGTATTACCACTGTCAGCAACAGCGACATCATCATGATAAACGCAGTGCC
CGTGCGCAGCGCGATGACTGGCTGAAGAAAGAGATACTGCGCGTATACGATGAGAATCATCAGGTAT
ACGCTGTGCGTAAAGTCTGGCATCAGTTGTTACGGGAAGGTATCAGAGTGGCCAGATGCACTGTGGCA
CGTCTCATGGCGGTTATGGGGCTTGCCGGTGTTCTCCGGGGTAAAAAGGTCCACACTACCGTCAGCCG
GAAAGCCGTTGCCGCAGGCGACCGCGTAAACCGTCATCAAGGCAATATGCCGCGTACTCCGGGTGGCC
CGCAGCGGCTGGTATACGTGGTGTCAGCGGCGGACAAGGATAAACACACGTCAGCAGTTCCGTCAGC
ACTGCGACAGCGTTGTCCGCAAGGCTTTTACCCAGTCCAGCGTTACGGTGCCCCACGCCTGACGGATGA
ACTGTGTGCTCTGGTTACCACTTTAACGTAAAAACCGTGGCAGCCAGTCTGCGCCGTCAGGAACTGAGC
GCAAAAGCCTCCCAGAAGTTCAGCCCGATCAGTTACCGTGCACACGGCCTGCCTGTATCAGAAAATCTG
CTGACGCAGGATTTTTACGCCAGTGGCCCGAACCAGAAGTGGGCGGGAGACATCACGTACTATTACAG
CTCACCGACTGCGGGGAAACACGGGGCGCCGGGTTATTGAAGGTGACCTGTCCAGCTACTTCGACACA
GTACATCACCGGCTGCTGATGAAAGCCGTCTGCCGCAGGATCAGTGATGCACGTITTATGAGACTGCTG
TGGAAAACGCCGTGTTGATGTTGGTCTCTTCCGGGCGGTCAGTGAAAGTATGCCACAGGGCGGTGTGA
TCTCGCCGTTGCTGTCGAACATCATACTGAATGAGTTCGACCAGTATCTGAATAAACGCTACCTGAGTG
GAAAAGCCAGAAAGGATCGCTGGTACTGGAATCACAGTATCCAGCGAGGCCGAAGTACTGCTGTCAAA
GAAAACTGGCAATGGAAACCTGCCGTTGCTTACTGTTGCTATGCCGATGACTGTGTGCCACGAAGGCG
CGTCCTGGGGACGTGAGTCCCTGTGCAGCCATGCGGCACAAGAGATGAGGGAAGGCCCCTCGGTGTC
GGCGTGCAGGCGCAGGCACCAAACCATCCACTGCTGCTGCATTTAAGAGATGTGGTGGTGAGCGAGAT
GGGAAAAGGTCGGACGTGAGCCGATCAGGTATGGTTCGAAGAGACGAACGAAAGTGAACCGCTGAT
GATGTGTCGAAAGCATATAGATGATGTCAAAACAATGGTGGAGTCGTTCCTGTGGGATATGTTTACTGC
CTGCGCGGCATCCGGCATAAAAGCGGCGTGAAGTCGATTCAGGCTCTTGTGGGGAACGTGGACCTGTC
ACCCCGATGTGAAGGGAGAAGCGCAAGTGGAAGCCCCATGAGCGCCAGAGTACCAATGCGGGGTACA
GGGATGGAGCACCAAGCGATTGCGCAGTAAACATGCTTGACAACTTAATTGGGGCCCCCTCTTCCTTTT
ATGGTGTAATTGTTTGATTGAATACTTCCGGGGATTTCAGTATGAAATGAAGTATATTTAATATACTTTA
CAAAAAGTTTGTATTAGTGGAATAAGGACAATAGTTTATCCAAACTGCCAAGCCCCTTTATTATAAGTA
AGATCTGGTACTAAATAAGCGTTCTAAACCGCCCCGAGTAGGCGGCCTGACGGCTGCGGCTTGTCATG
GCCTGTGATTACCGTTATAAAAACAGACAATATCATTGTCTTTCAGGTAGTTATATGGCCCGTTCAGCTA
AACCACGCAAACGAAAACCTGCACCTCAAAGAAGCAAACTCCTCCGCTATGTCGTAAAACTTCACGAAG
ATGATTTTTTTGACGAAGAAGAAGCTGAGGTTCTGCGCTTTGATAATTTTGACGATGCCGTTGAGTGTT
GCGCAGACCTGAATATTCCGTTCTTTGTGGATGCCGGAAACAAAAAACTGGTCTTCTGGTTTGTACGTG
TTGATGACGAAGGGTATCCGGAAATTGCCCGCTGCACGGAGCGGGAATTTGCGACAATTCCTGCCGGT
ATCAGCGCCGACGGCATGTATTGCCCTGAATGCGGTACGGTTCACTGGCCGGATGGCGTTATTCCGCCT
TTCTGATGCTTCCCCGTTTTGCCGATATTTTTCAGCAGGGAAACCGATGGCTTAACTGGCTGGAGAAGC
AGCCGGTGCAGATGAGCCGCCTGGAACATTATGCCGGTCAGGATGAAATTGGCCTGCGTTACAACAGC
CACCGGACAAAACGAGAAGAAAACCTTGTGATGAGTGGCGACGAGTTTATGGAAAGGTTCTCCTGGCA
TGTGGCGGATAAGGGGTTCCGGATGGTGATACGGGGTCCTGAGTCCGGTGAAGCGGCGATTACTGGA
AGATGTGGTGTACGCCATAACGGAGACAGTGAGAAAAACGGCGAGGCAAATCACAAGGAGAGGGAT
GTATCAGCAGTGACTGAAGGTTGACCCGCTGAAGTGCGAGCTGTGCGGAGGTCAGATGCGGTTTACG
GGGCTGAAGAGGGGTTACCGTCTGGCAGAGCAGGTTATGATGCATGAGCCGCTGGCTCGAATACGGT
GGTGCGGCTGAGAGTCGCAGAGGGGAAGTTGCGTCCATGTTGAGGGGAACGGAGCAAAAAACCATC
AGAGATACATTGAATCAATCAATGTAAGCAATATTAAGTGTCTATACCGATTACTTGATGATCAGGTTG
TGTTGAATAAACGCGCATTCTTCGGTGCTTTTTTAATCTTTTGGGGATTTAAATTCCTATCGATGGACAT
GAATGCAGGCTATATAAGAGCAGCGCGCATCCACTTACCCAATGCGGTTGAGAAAATAGCCTTCGATC
GCTTTCATGTGGCGAAACAACCGGGTGAGGTGGTGGATAAAACCCGCCAGAATGAACCGCCCCGGGTT
TCCTGGAGAGTGTTTTATCTGTGAACTCAGGCTGCCAGATCATCGTTTCCGATGGAAGCATAATAAGCT
TTTTCTGCTTCTGCCGGAGGAGTATGGCCCAGCCTTTCCAGCAATCGTCGATTGTTATACCAGTCCACCC
ACGTGAGTGTGGCCAGTTCCACTTCTGCACGGTTTTTCCAGCTCTTACGGTGTATTACCTCCGCTTTGTA
AAGACCATTGATGCTCTCCGCCATCGCGTTGTCATACGAGTCGCCTGTACTTCCTGTTGATGCCAGTAAT
CCGGCTTCCTTAAGCCGCTGTGTGTAGGCCAGCGATACATACTGAGAACCTTTATCACTGTGATGGACC
GTGCCGGACGGTCGACGGGCCCATAACGCCTGCTCCAGTGCATCCAGCACGAATGTCGTTTCCATGGA
CGATGAGACCCGCCACCCCACGATGTATCCGGCAAACACATCAATGATGAACGCCACATAGACGAAGC
CCTGCCATGTGTCAACGCCACGATGTTTGACCGTTATTTGCCATTTTCATGCCCGCTAAATACTTTCGTTA
CTGATGCTGTCCGCTTTCCACCATTTCATCATCACTGATTGGCGTTGTGTTTTTGGTCAGCACTCCAGCTT
TACGTTTCTCTTTTAACCGGTAACTCTCTCCTTTGATATTCAGCGTGGTTGAGTGATGTAGCAGCCGATC
CAGGATCGCTGTTGCCAGTACGTGATCTCCGAACATTTCTCCCCAGTCTGCGAACCCTTTGTTTGACGTC
AGTATGATGCTCGCTTTTTCATATCGACGGTTCAGTAGCCGGAAGAACAGACTGGCTTCCTCTCTGTTCA
TCGGCAGATAGCCTATTTCATCCAGGATCAACACCCGGGCATAACTCAGTTGCTGTAGCTGACGCTCCA
GCCGATTTTCCTGTTTCGCTTTCATCAGTGTCGCGATCAGTCTGTCCAGTGGCATAAACAGTACCCGATG
TCCCGCATCCACCGCTTTCACGCCAAGAGCTATGGCCAGATGAGTTTTTCCGACACCAGGAGGGCCCAG
CAGGATCACGTTTTCGCTGCGCTCCACGAACGCCAGACCAGCCAGTTCCCGGACAACCTTACGGTCGAT
GCCCGGCTGGAAGGTAAAGTCGAACTGCTCCAGCGTTTTGACCCACGGCAGACGAGCCTGCTTCAGCC
TGGACTCCATACCGCGCTGATGCCTGCCGTTCCATTCCTGCTGTAGCGCCATGCACAGGAACTCCCGGT
AGTTCAGCTCTTTTTTAGCTGCCTGTTCCAGCAGGCTTTCAACGTGATAACTCAGATGCTCCATTTTCAG
GCGACTCAGCAGGACTTCCAGTTCATGCATCACAACAGCTCCTCATAGGCACTCAGTGGTCGATGTTCC
ACCTGACTGACCTGCTGCCAGAGCGGGGCGTGATGCTCCGGCACTGTCTGCCAGCCAGACGATGCTGA
ACAGAGGCGATGTGAGGCCACCAGTTTCTCATTACTGTAGATCCGCAACTCGTCATCCAGCGATATTCG
TATCGATACCGGCTGACCACACAGCGCTTCGGGAACGCTGTAACGATTACCACCAACCTCGATATAGCT
GTCCCAGGACACATGGCGGATATCGAAGTAACTGGTATCGAAGTCTGTATCCGGTAACGGCTGCAGAT
GTTCCTGCTCCAGCGCGAAGCGCTGTTCCGGCGTTTCTTTGAACTGGCGAAGCTCCCGTTTGTCAGCCA
CATCGGCTATCCATTGCTCCAGTTGTTGATTGACATGAGTGAAGCTGTCGAACCTGCGGTACCGGACGA
AGAAGTTCTCCTTGAGGTATTTCACCATCCGCTCAACCTTACCTTTTGTTCTGGCCCTGCGTGGACGGCA
TGCCCGTGGCAGGAAGTTATAGTGGTCAGCCAGCAACAGGAATCCGGAGTTGAACACGACTTTCCCGT
TGTTATTCTTCAGCACCGCAGCCTTCTGGTTATCAACCAGCACCGTTTTCACACAACCACCGAAGTAGCG
GAAGGCGCGAACCAGTGATTCGTAGGTATGCTCAGCATCCTGTTTTGGTGCGGCGAAGACATGGAAGC
TGCGGGAGAACCCCAGCGTATTAACCGCAAAGTTAACTTTGCACCGTTGCCCGGCAACCTCCACCTCAA
CTTCGCCCCAGTCATGCTGGAGCTGGTATCCAGGCTGAGTTTCGAAGCGAACTGTTCTTTTTGACGGAC
GCATTTTACGTTTGGGCTGGATGTAGTAACGCAGCATGGAACGTCCGCCCGTATAACCCATTGCCTTAA
TCTCCGCGAAGATAACCTCGCTATTCCAGACATTCTCTGCCAGGCGCATGTCGATGTAATCCATAAACG
GTTTCAGCTTAACCATTTTGTGGCGGGTCTTTCTGGCTGGCGGTTCAGGGTATTTGAGGTAGCGTCTGA
CCGTCCGTTCAGAGCAACCAATCTGAGTCGCAATATCGACGATGTACGCGCCCTGCTGGCGCATTTGCT
TTATCATGTAAAAGTCCTCTCTGCTCAGCATGTTGATGTCCTTTCTGGTGTGAGAACCTTAAGGAAACAA
CATGTTGGGTGGAGTGGACAATCCAAATGGCGAATTACCGTCTTATATCACTGGCGCTGACACATGTGC
TGACGTAAGTAAAATCAGCCACCCACAGCTGGTCAGGTCGTTCTGCCACGAACTGACGGTTTACGCGGT
CGCCTGCGGCAAAGGCTTTCCGGCTGATGGTCGTACGGACCTTTTTACCCCGGAGAACACCGGCAAGT
CCCATAACCGCCATGAGACGTGCCACAGTGCATCTGGCCACTCTGATACCTTCCCGTAACAACTGACGC
CAGACTTTACGCACACCGTATACCTTGTGATTTTCATCGTATACGCGCTGTATCTCTTTCTTCAGCCAGTC
ATCGCGCTGCGCACGGGCACTGCGTTTATCCGGATGATGTCGCTGTTGCTGACAGTGGTAATACGTTGA
CGGGGCAATATGCAGTTCGCTGCATAGCGGTCCGACCCCGTACTGCTCACGCAGCTTATCCAGCAGTG
GCATCATTTTTTCCAGAGGCGGTCAAACTCCGCCTTCGCAAAATAAGCGGAAGCCTGGCGAAGGATATC
GTTACTGCGGCGCAGTTCACGATTTTCACGCTCCAGCTCTTTCAGACGCTGACGTTCAGCGGTGGTGAG
CCCTCCATCACCGCCCCCGGTATCCCGCTCATGCTGGCGAACCCAGACACGCAGAGTCTCCGGCGTACA
GCCAATCTTTGGAGCAATGGAACAAATTGTCGCCCATTGTGAGTCATATTCGCCCTGACTTTCCAGAACC
ATACGGACTGCCCGTTGACGGACTTCGGGGGAAAAACGAGTATTTTTAGTCATCCTGTTTACCTCTTTCT
CAGGAAGTTTAGTCTCCAGGATTCCCGGGGCGGTTCAGAATGAACATCCGCATCTTCCTGTTGAAAGTC
GTCGTCAGGCAAAGGGAACTCGCTTCCTGTGGCAGCACAGCGATAAATGGATGACCGAATCCAGGCA
GGAAAAGCTGATATGGCTGCGTGCGCAGATGAAGCTGACGAGCCTGTGCTGGGCGCTGAAAGAACTG
GCAAAGGATATCTGGAGCAGACCATGGAGCGAGGAAAGACGCAACGACTGGCAGAGATGGCTGTCG
CTGGCGGCCAACAGTGACGTCCCCATGATGAAGAATGTGGCTAAAACGATAGGGAAAAGGTTGTACG
GTATCCTGAACGCCATGCGCCATGGCGTATCAAACGGAAATGCGGAAGCACTGAACAGCAAGATCAGG
CTGCTGAGAATAAAAGCCAAGGGCTACCGCAACCGGGAGCGCTTTAAGCTGGGAGTGATGTTCCACTA
TGGGAAACTGAATATGGCGTTCTAAGCCTTCCCACCATGATCGGGGAAGACCCAGATATGAACAGGCA
CATTCAGAGTTACTTTAAAGAGCTTACTGAACGTCTGAGGTGTTAGAGCCATGACTGCCGCAGCATTGC
TGGCGGAAATGCCTGAATCGAGCAGCCTCTCGAGACGTGAAATCAGCGCACTTGTCGGTGTTGCTCAG
GTAAACCGGGACTCGGGAACTCTGCGCGGTCGACGAACCATTTTCGGTGATTGTGCTGGGGAAGAACA
GCTCTGTACATGGCGGCGCTTGAGGCACCCCGGTTTAATCTGGTGATAAAGGCGTITTACATGCGCCTA
CTTGCAGCCGGAAACGCCAAAAAAGTGGCACTGGTTGCCTGTATGCGTAAGTTGCTGACCATTATGAA
CGCAATGCTCAGAAAAAACGAAGAATGGAATGAATCGTATTTATGAAAACTGGATTGTGGGCCATAAA
TATTCCAAAAAGATAGCCCGCTCCGTTTATTACCGGAACGGGCGAGAGCATGACATTGTCTGTAATTAT
AATGAGACTGCTGATTCTACTTATTTTTAATTTCAAGAAAAGCGTTTTCTACAAAGAATTATGAGATTTA
ATGTTTTGTTTAGACAATATTATCATTCTGTTCAATAATGAAAGTCTTCATCAACAACAAGTAAAAACAA
CGTCCCTCTCATCGAACCGGACTTGCAGATTTCCCGCATCCGGCTCTCTTGCAACACATCAGACCTTCAC
GTTCGGGCGGTTATGGGGAAGGTCCAGCAAGCTGTATGAGCCCTAACTGTTTGAATACCAGTAGATTA
CCAGAAACTTCTATCGATTGAAGACATTACCAACTAGCGTTACTAAATGTTGACCAAGTTTTGTTCACTT
GTCTGGTTTTGATAGTAGCATTCTGCAGAGTATTTTGATATGATAAATATGCATTAACAATGATATTATT
GTTAATTTCCTAGCATGAAAATTAAAAGATCAACTTTTTATCTCGAATATTTTTTACATTATCAGTTGGTT
TCTTATGAATGACAATTCATTGCTTAGAAATTCTTCATTATTCATCGCCTATATGGGATGTGTAGGGTGG
GTCAGTGCTTATTCTTATGGCTGGGGAACCTCTTTTTACTATGGATTTCCATGGTGGGTTGTTGGTGCAG
GTTTGGATGATGTTGCCAGAAGTTTACTCTATGCTATCATAGTAATGGGTATACTTTTTACCGGTTGGGG
GATTGGTATTTTATTTTTTCTTCTTATAAAGAAAAGAAGTAAAATACAAGACTTGAGTTTTTTTAGACTAT
TTTTTGCTATTACCCTACTGTTTTTCCCTGTAATATTCGAACTTTTAATTCTCAAACAATATTTCATACTTCC
ATTATCACTTTCATGCATTATTAGTTCATTGGTTATTTCAATAATAATTCGTATATATGGACGGATCTTTT
CTGTATCATGTTTTTCAGATATCCCTTTTGTTAGAGAACACCGCATTAAGTTAATTATGGCTGGTTTTTTG
GTTTATTTTTGGCTTTTTTCCTTTCTTGTTGGCTGGTATAAACCTCAATTAAAAAAAGAATATCAGATGCT
TTGTTATAATAATAGTTGGTATTATATTCTTGCTCGTTATGATTCACGGCTCGTTTTGTCTAGTTCATTTA
AAGATGACAGTAACAGATTCTTAATATTTAATACAGAACAGTCGGGATTTTATGAAATCAATGATGTTT
ATGTCAGAAAGTAATAATCCTCTCCCACAAAGTGAATTAAATGAATATATAATGCGTTCGAAGTATATG
TATTGAGCTGTCATACTGAGAAACAAACCTTAGGGCAGCACAATTAAAGCCAGAAACAACTATGCAAG
AAAGTTACTAAATAAGTGCTCGGAACAGTAAAACACTCAGGGGGGACTCAGTCCGGATTATGTAATCA
GATAAATTGCCAAACATCACAAATCACCAGCCGGACTGAGTGATGCCGAGCATTGCACCAATACCCCGT
GATGAACGATGCCTGATGCAGAAAGCTCCCTATAAAACAAAGAACAAAAACTACGACCGGCGATTCAC
CGTCATACTGATGCTGCATCGTGGTGACACTGTCTGTCACTTTCATGTTGACTGAACAACTCTGCTGCGA
AGAAGGTGTACCGCCGGCGGGATTTGATTGTCCGGCGTCTAGCTTCCACTTCCCTGATGGAACTGAAA
GTTGTTATGGCCGGTCAGCTTGCCCTGTCCGCAGGACATGAGGTGCTGTGACGGACAGCACCAGTAAC
GGAAAGTGACAATGAAAATGAATACTGCGCTGATAGTTGCACTAATGTGTATGTGGTATGCGGTACCC
GCTGCTGCAAAAGAGACTTTACTGGCTATGCCACGTAATTCAACAGAACATTGCTATGCGGAGATTAAT
GTGCACGGACCATATGGGGTGTATTTCCGAGTGGTCCCGCATCCCCCCGGGGGAAAGAGCTGGGTCGA
ATGTAACAGTGACTACTATTACTCTGATAAACCACCGGGAGTACAGATACTCGGAACCCGTGCAGGGT
GTCGGGTATACGGAATCTGCGGGACCACCTCGACACTGCATGTGGCTGGAAGAGGGGTTGTGTGTATT
AAAAATATATGCAGTCCACGTGGGATGATAATTCACAGGATACGGAAACGTCCTGTAGTGGCTGTAAG
CGATGAGATGTGATCTTACCCAGCAATAGTGGACACGCGGCTAAGTGAGTAAACTCTCAGTCAGAGGT
GACTCACATGACAAAAACAGTATCAACCAGCAAAAAAACACGTAAACAGCATTCGCCTGAATTTTGCAG
TGAAGCCCTGAAGCTTGCTGAACGCATCGGTGTTGCTGCCGCAGCCCGTGAACTCAGCCTGTATGAATC
TCAGCTCTATGCGTGGCGCAGCAAACTGCAGCAACAGATGACCTCGTCTGAGCGTGAAAGTGAACTGG
CCGCTGAAAATGCCCGCCTGAAGCGGCAACTGGCGGAGCAGGCAGAGGAACTGGCTATCCTCCAAAA
GGCCGCGACATACTTTGCGAAGCGCCTGAAATGAAGTATGTCTTTATTGAAAAACATCAGGCTGAGTTC
AGCATCAAGGCAATGTGCCGCGTGCTCCGGGTGGCCCGCAGCGGCTGGTATACGTGGTGTCAGCGGC
GGACAGGGATAAGCCCTCGTCAGCAGTTCCGTCAGCACTGCGACAGCGTTGTCCTCGCGGCGGCCTTT
ACCCGGTCAAAACAGCGTTACGGTGCACCACGCCTGACGGATGAACTGCGTGCTCAGGGTTACCACTTT
AACGTAAAAACCGTGGCGGCAAGCCTGCGCCGTCAGGGGCTGAGGGCAAAGGCCTCCCGGAAGTTCA
GCCCGGTCAGTTACCGCGCACATGGCCTGAGGTGTACTGGCAATAGCGGACACCACCATTTGTTCTTTT
TCTAAGCAGTCATCTGATAATATTTTATCCTGAAGGCTGCCGGGGAGATATTCCCCAGACGAGAGTGAC
GACGCTGACGATTGTAGAAAATCTCAATGTATTCCCGTATTACTGAGATGGCCTCATCCCGGCTCTTAAA
ACGATAGTGGCTCAGGCTTTCATTTTTCAGCGTTCCCCAGAAGCTTTCCATCGGAGCGTTGTCGTAACA
GTTTGAGGTAGCCTGAGTGCGTATTCCGCTCTATTCGATCACGGATTGGCATCGAAGTTCGATCAGCGA
TTCGCATCCATTCGATCAACCGGGAACTTAACTTTTTCTACCGCTTTTTAACTCGTTTTTTCGCTGCTTTCC
ACTGTAGGGGGATTTTTCCTCAGGGACTCTCCCTGAAGGACCACTCTGTGTGAGTTATGTACCAGTCTG
TCCAGGATGGCATCTGCTGTTGTTGGGTTCTCCATCAGACCATGCCATTTATCTACCGGGAACTGGCTTA
CCACTATCGTGCTGCTTTGTCCGTAACGATCCTCTGTGATCTCCAGAAGGTCATTACACTGGGCGTTACT
TAACTGTTCCAGCCCCAGATCGTCCAGGATTAGAACCTGTATTTTTTGCAACTGTTTCAGATATTTCAGC
CAACTGCCATCAACACGTCCCTGAGCCAACTGTTCCAGTAATCGCCCGGTTCTACAGTACCGGACGCTG
TATTTCTGTCGACATGCCTGTTCGCCCAGCGCATTGGCTATCCAGCTCTTACCGCATCCTGTCGGACCGG
TTATCAGTAGGTTTTTCCTGTGGATTATGTAGTGGCCGTTCAGCAGTTCCCTCATCTGTTCAGCCCGCAG
ACCTCTTGAGGCCGGATAACGCAGCCCTTCCGGTGTTGCCTGATATTTCAGGCAGGCATTTTTTCTCAAC
CGTGCCACACGACTGTTTTCTCTGCTCAGAAGCTCGTCATCAACCAGCAGTGTCAGCCTTTCCTCGAACG
ACAGTTCGTCGTAAGTCTGAGGCGTTTCCCGTTGCCGTATCAGGCTCCCGGCCATCGCCGGGAGTTTCA
GTTGGGTCAGTTTGTTTAACAGATTATCACTCATACGGTTGTTCCTTCTGTCAGTGGTAGTAGCCAGGAC
CACGCAGGTTTTCATGCTCTGAAGCATATGCCGGATGTTGTTCGCCCTGGCGGGATAACGGAAGCTCAA
CGTTATCCCGATGATGACGCAGCAGATTGTCGATAAAGCGTCTGTCCGGGGCGTTATAGTGCAGGGCA
ACGTAACAGGCCTTTTCCAGCCGTTCCGGGCCGTATTTTTTCTGAAGTGCCAGCAGACCCAGGACTGCA
CGGTAAGCCAGCTCCGGATGGGGTTTGGCTTTCAGCATGGATTCAACCACCCGGCCTGTGGACGGACC
TGTACGGTTTCCCTGCTCGATAAGGCGTTCCGGTGTCCATGTCCCATGCCGCCGGTGGCGTTCTGGCAT
GTGAAGCGGATTGGTTGTGTGTTTATATTCATGTGTGCTGCGCGGATGTTGCGCCACACACTGCCCCTT
GTGCCAGAGCTGTACCACCGACTGCCCCACCTTCAGTGACAGACGCTGACCGACCAGTTCGTGTGGAA
CGGAGTACCAGTGACGGGCATATTCAACGTGGTAGTCAGGGCCAACCTTCACGGCTTTGTATTCCGTGT
ATTCATAAGGCTCCAGAGGCAGCGGAGAGAGTGCTGGTGCATCCAGCATGCGGAAGCGTTCTGCGCG
GGTTTGATTACCGTATCCCTTCATTGGTCGGTTATTCATGTCCGTCAGCAACTCCCGAAGGCGTGCATTC
AGCGCCCTGAGCGTGTGGAAGGTTTCATTACGGATGCGGGCAAGAAGCCATAGCTCGACGATGAGGA
CGCCGTTTTCTGCTTTTGGTTTATCTTTAGGTTTGCGCGGGCGGGCCGGGATAATAACCGTGCCATAGT
GCTCAGCCAGCGCCTGATAACTGTCGTTGATGACAGGCTCATAGCGATCGGCTTTTTTCACTGCGCTTCT
GAGGTTATCGGGGATGAGCAGCTTCGGTACGCCGCCCAGGAAGGTCAGGCAACGGCTGTGGGCATTT
AACCATGACATCATGTCCTGACCTTCGCACGCTTCAACATAGGTATAGTTGGACGCCCCCATTACAGCCA
CGAAGATTGCCACGCGGCGTATCTCTCCGGTGTCCGGATTAATAACAGGGACCGTAGGACCACAGAAG
TCGATAAAGAGCTTTTCGCCAGCACGATGCTCCTGTCGCATGGAGAGGCGTCGCGTTTTTTTCCACTTCC
GGTAGCAACGGCAGAAGTGTGAGTATCCCATCGCTTTGTCACCCGCCTGTGCCTTGTATTCCATCCACA
ACAGTTGGCGCGTCACGCCAGGTTTTCTCATTTCCGTATCGAAGTACAGCATGTCAGGCATAACAAGCT
CTGACGCAGAGGTGTCTTTGGGCGGGAAAATGAGTTTCTCCAGCGTGTCATGGGATATATCCGCAGGT
AATGGCCATGACAGAGAAGAGGCTTTGAAGCGGGCAAGAACCTCAAAAACGGTTGAGGCGCTGATGT
GTAACGTCAGTCCGATACAACGGTTAGTCAGCTTCTTATCGAACTTGAGCTGAAGGATCTTAAAACAAG
TTTCCATAGTGCTCCTGGATTTTCTCTTTTTGGACATTGACGTCTCCACTGGTTTAAACACGGCAATGGA
GACTGTGGTGAAAAGAGTTAATTCCCGGAGTGACTGGCGAATTCCAATCAATGATCGGAAGTATGCCA
AATCGTGATCGATTGTGATGCCAAACTGTGATCGGAGATGGATGCCAAATCATGATCGATTCGAATGCC
AAACTGCGATCGATTCAGATGCCATTTTGCAGCCTGAGTTTAACGGACACTCCTTCCTGAAATAGAATG
GCATCAGAAGGAGCCAATAATGAGCAGAAAAACCCAACGTTACTCTACGGAGTTTAAAGCCGAAGCCG
TCAAAACGGTTCCTGAAAATCAACTTTCGATCAGTGAAGGCGCTTCCCGATTATCTGTTCCTGAAGGCA
CTTTAGGACAATGGGTTACCGCCGCAAGAAAAGGGCTCAATACACCAGGCTCCCGCACAGTGGCTGAG
CTGGAATCTGAAGTTATGCAACTGCGTAAGGCTTTAAATGAAGCACGACTTGAGCGAGATATATTAAA
AAAAGCAACAGCGTATTTTGCCCAGGAGTCGCTGAAAAATACGCGTTAATCGAACAATGGCGACAACA
ATTTCCCATTGAAGCGATGTGTCAGGTATTTGGTGTATCCAGGAGCGGTTATTACGACCGGGTACAGCA
TGCCCCCTCAGACAGAAAACAAAGTGATGAGCGGCTAAAACTGGAGATTAAGGTGGCACATATCCGCA
CTCGCGAAACATATGGAACCCGGCGGCTCCAGACGGAGCTGGCAGATAACGGAATTATCGTCGGTCGT
GACAGACTGGCACGTCTTCGTAAGGAGCTGAGGCTGCACTGTAAGCAGAAACGCAAGTTCAGGGCGA
CCACAAGCTCTGACCACAATCTGCCGGTTACGCCGAATCTGCTGAATCAGAACTTTACGCCCACAGCAC
CTAATCAGGTCTGGGTCGCAGATATAACGTATGTTGCCACCCGGGAAGGATGGCTGTATCTCGCTGGC
GTCAAAGATGTTTATACGTGCGAAATTGTCGGTTACGCCATGGGAGAACGCATGACGAAAGAGTTGAC
GGGTAAAGCCCTGTTTATGGCGCTCAGGAGCCAGCACCCACCTGCCGGGCTAATCCACCACTCCGATCG
AGGTTCACAGTACTGCGCATACGATTACCGGGTCATACAGGAGCAGTCTGGTCTGAAAACATCAATGTC
GCGTAAAGGTAACTGTTACGACAACGCTCCGATGGAAATCTTCTGGGGAACGCTGAAAAATGAAAGCC
TGAGCCACTATCGTITTAAGAGCCGGGATATTTCGTCAGCATACGGGAAAACTGATTGAGGTCGGCCTC
AGTTTTGAGGCCTTTAGCCAGTTCAGCCGCAAGGGCTTTAAGCTTCTTTTCGTCCATAATTTACCTGTCTC
CGTTGTTGGAGTAAACATATCAAAAACAGGCAATTACACAATTTAAATTACAGTCTCAAAACAAACCCA
ACGGTCAATATAGTTTACCAGCAAGCTTACTAGCAAAGTGCTAATCATTTGGTATCTTTCGTGCTATTCT
CCTTTTACGTAAGTACTTAAGGGGAACGTATTTACTACTTTTAAAGAAAGCTCCGTTACTATCTGAAATG
CAATGAGATAATAAACGATTACTCTCGGTTAGTATCTCATGATCATTTACAGACTTTGCATAATCGCGAA
TACTATATAACAGAGTTTTAACATTACAGGATGAATAGTCTCTCTTATCAACTCCTAATAAAAGAAGGAT
TATGGTCAGTCGTTTTTGCTGTCTTATCATATATTATTTAGTGCAGTTCCTAACAAAAACATAACAATTTT
CTACAATTGATCCTGTGACTACATAAATATCTTCAACAAAAATATTTATATCAGCAAAAGAGTTATTATG
AATGATTATAAATCATCAGGTGTTTTTTCAACGACCAACTCCAGAGCACAACGATAAATGTCTTTTCTAA
CCAGGTCACATTCAGATGAAATCTTTCTGAGTAAAACATTTATTATATCCTTATTGCTAAGGAAAGATAT
TGGGTTCAATACCTCATTAGCAATGGAGTTAATGATGGAAGATTCTATTTCAAATTCAGACTGATCAATA
CCTATTTTATTACAGAAATTAACTCCCATATAACACATCTCCTTTACTTATTAATGAGTGTTTTCAACCTGC
TCAAGCCAAACAATAAGACGTAGGACTTCATCCAAGTGTTCAAAATCAACAAAACTATATTTTGTATGT
GTTTTATATAGTTTTCTAGCTAATTTCACATCACGCACAGTTGGTATACCAACTTCATTTGCATATTTTCT
GACAGCCAAGGCACACTGGTTAGTTTCAATGAGAGAAATAAAAGGTGCAGGCGCTATTTCTGGATTAA
AATAAATACCAATTGCAATATGAGTCGGGTTCATTACCACTAATTTTGAATTACGTATATCAGATTTAGT
CTGCTCTGAAAGAATCTCGATATGCAACTCACGCCTTCTCGACTTTGTCTCAAAATGTCCCTCTTGCTCTA
TATATTCTCTTTTTATCTCCTGTTTATCCATCATCATATCTTTCATGTATAAAATGAACTCAATCACAAAGT
CAAGAATAATAACAAAAATAGAAAATGCCAAGAAAAATAATATTATATCCTTAAACAGCCCCCCCCAAA
TAAGATATAAGCCATCAACACTAGAAAACACCTGAGAAAAAATTATTTTTCGGTCATTAATCCAAAAGA
AATAGGTTGTTAATGCTAGAATAATAAGAAGCAGAATGCTTTTGAAAAATTCTTTTATTGTCTTTATACT
AAATATTTTTTTTAACCCTTTAACAGGGTTTAATACTGAAAAATCAATCTTGATAGCTTTAGTCGCAAGA
ACAAACTTTGTTTGAACCAATGTTGGCAACACAGCCGAAAGAACACAGAAAAAAAGTGGGAAGCCAAT
TATCTTAAAAAAGACAATAACCACTGCAAAAAAGTATTTACCCTCATTAATTTCGAAGTCATTAATTATT
ACATATCTGTATAAGAGCATTACATCACTTAAGGAAAAGAATGATATTATAGTAAATGTCCCTACCAGA
ATAATAACAACAGTCGTTAAATCCTTAAATTTAAATGACTGTCCTTTTTTTGCGGCATCCTTTAGTTTTTT
AGGTGTCGGCTTTTCTGTTTTATTTGCCATAATATTTTATCTAACAAATAGATTTGTGAAAAATTTATGTT
CACCGAGGAAAAATTGAACTTTAGAAAAGTAAATAGTAGAAGAACAGATGAAAATAATAAATATTGCA
AGTAAACTTTTAATAGTTAATGATACGGAAAAAGCATTCATCTGCGGAGCAAATCTCGATAATACACCA
AGTAATACTTCTGATAGTAACAATACTATCATAACAGGACTGGCTAAAATAACAGCCTGACTTGCCAAT
AAAGTCAGAAATGTTAAGATATTTGAGACACGGAAAGAACATTGAGAAAATAACGGGCATATATTATA
AGACAATTGTATGGATTCTAAAATAAAGACCATACCACCACTGTATAGAAATACAACTGCAGAAAAAAG
ATTGAAAAATTTTGCCAACTCAGACGTATCAACACCATTGGCAGGATCAATTGAACTACTAAGCGTTGC
CCCACGCTGGTTGTCAATAATGCTACCAACAGCATGAAATATCCAAAAGGGAAGAGACAAGCAAAAAG
AGAGAAGGAGGCCAACAATTATTTCCTTGAACATAAGGAAATAAACATGTTCAAAAACAGAAGAACCT
ATGTCTACCTTACCAGAAGTAATTAATCCTGATGCAACAAGAAAAATCACAGGTATTCTAATAGATGGA
GAAATTATACCGTTATTTAAAAATGGAAGAAAGAAAAATAATGGAGCCAATCTAAAAAAAACGCCGTT
CAGGAGTATTAAAAACACATGAATACTTTCGAACCAGCTTGAAATGTCCATCAAACCCCACTCTTAATTA
AAAACATTATTTCATGACAAAAAGACAATAAAACCTCACCATACCATCCAGAAAGAAGTAGCAAAGATA
TTGAGACACCTATAAGCTTTATACCAAAAGGAAGTGTCTGCTCTTGGAGTTGAGTCACTGTCTGTAACA
AACCAATAGATAATCCAATAACCGTAGCTATACCTACTGGCCATAAGGAAAAGATAAGGATTAAATAAA
GAGCCTTATTACCCATATAAACTATATCAGACACATTACCTCCATCATCTAAGCAGGAACATTGATATAT
TGTTCAATCAAGGCCTTAGACAGGATACCCCACCCATCAAGCGCAACAAATAATACAAGTTTTATTGGT
ACTGATATTGTGATTGGACTCATCATCATCATACCAAGTGCAAGTAAAATGCTTGAAATAACAAGATCA
ACAACAACAAATGGCAAATATAAATAAAAACCTATTTTGAACGCATCTTTGATTTCACTTAGCGCATATG
CAGGCAAGAGGGAAAACAATGAATAATCATTATTCTCTGCACTCTTTAAATCAGCATTTTCTTCTTCAGA
CCTTTGGAAAAAACGTGCTAACTCAAGATCTGTATGCTTTTTCAAGTATTGCTTATATTCCATCAAGCCA
GAATCAGAAAAACGGACAATATCAGATATTGTATCAAATTTTTGAGGGCCATTTAGATAATTTTCATAA
CCAGCTTCAATAATGGGTTTCATCACGAACAAAGCCATAATCAATGCAATACCATTGAGAGTCATATTT
GATGGAACCTGCTGCAAACCAAGTGCATTTCGTACCATTACAAAAACAATAGAAAACTTTATATAACAG
GTACCAGCAGCAACTAAAAATGGTAATAGCGTAAAAAAAGAAAGTGTAGCGATGAGGGACATGTCAC
TCAGCATGAGATTACTCCTTTACCATCCAAGAACTAATCTCGATACCATAACCATCCTCAATAGAAACAA
GTTCCCCATGCCCAACCAAAGAACCATTTACTTTAATATTTACATGTTTAACTATGTCATCGGGAAACTT
GAATAATTCGTTTTCTACATACATTTTTAGTTCATTGATTGTCATATTTTTTTCTAAAAGAATAAAGTCCAC
TTTTACATTGATATCATCATAATTGAATAATGCCAAAGAAACTTCTGTGTGTTCTGACTCACCATTACTTT
CTGACAGATTAATTTTTGCCTCATTATTATCATTCACAATATAATCCCCAATAATAACTTGATTACACAAT
AATAAATTATAAAGTTTCTGAACTATGATTATATCACCAGTGCGAATTCTTTTTAAAGTAGCCAGCCTTA
CATTAGATGAACCTAAGCAAAACTCTAAAAGATGATGCAATAAGTCAAGAGAAACTTGATTACGTAATA
CCGGCATCTTGTCAAAATAAATAATACCTAAAGAAGAGGAAAAACAGCATAAATGACTCCCAGTAGTT
GCATACTGGATTTCATTCGTGATCCGGAAAGTAATCTTATCAGACAGTTCTGGAATCGAAAATACAGAT
GACGACTCTATAAAGACACATTTTGATAAATGCAGTAAAGATTCTTGTGTAGAAAATCCTGAAAATACC
TCGTATTTTTTTTTCAAAAAAGATAATGTATCAATACGAATTACCCAATCCCCACACACGGAAGTAAGAG
TGACTAAACTTTCACTGCCTGTAAATTTATTTTTGAATGTCTGAATCGAAAAACGCTCGCAGAGTTGCTT
TGCATATAAAATCTGTAGTTTTTCGTTAGCGTCAAAATGTTTAATTCTTAGCATTCTTCTTCACTCTGTTCA
TTATATCCTGTACTATTATTTTTATTCCCCTTATTATGTTGACTGCTATCAAATCTATAATTACCCTCAGCA
TCTTGTTTCAAAATTAAGTCAAGTTTATTTCCTACAAATTGATCTGACGGTTTTAGAACAAAAGAACCAG
ACTCCACTGAAATACTAACAGAATGTCCAGCCCCCCACTGTTTAAAGTGGTAAGTCATTTCACTATTTTTT
TGAAAAACATTTTTATCATTTTCAACTATTGATTTTTCTTGCAATGACATGGATGGGGTTATGTCTGCAGT
ATTTTTATTCATTACAGGCAGCTCAAATGATTTAGCAACACTTCCATTTAGTAAAGAAATGATGTCAGCA
ATATTCTTATCAATAGACAAGCTATGATTTTTTAAACCAATTTGACCATTTCTATTATCAGATTCTAGCAG
ATTATCAGGGTTTACAGAAGAAGTATGACTTCTTTCGAAGTTCTTGTCTGCATTTCCACTCTTATTATATG
AAAAGATCCCCCTCATATCGCCGATATTTATTTTTTCCCTTGCTTTTATATCTTGATCTGGTATCAAAATAT
TTTCTTTAATGGCCATTGATTCTGGAATATCAGAATATGTCGCTCGTTCGTTTCTAAGTTCGAACGCATTT
TTAATGCCACTATAAATTTCTCCTTTATCATGCTTATGTTCAAAGTGCTTATCAATAGTAAAATAGTTAGA
TGCAATTAAAGAGTACTCAGGAATTTCAACCTTCCTCTTCTTCTTCAAAACGAGACTATCAATATTATCTA
TTGAAGATAGTTTCTCACATTTTTCTATCTGTTTGTCACTACTAAAATTTAGGTTTATATTATCTAATGCCA
TACTTCATATCCTCTTGTGATAACTCATCAAGTATCAACTGTTTGGCAAAGTTATTTCTTTTAATCAAGCC
AATCCGTTTACATATTTTACTCTCTTTATGAAGCAGTTTTCTTTTAGAAGCATTTGCCTCATCAATATCTTT
TTGAATTTCTTCAATTCTTCCCTTTAGAAGAATTCGTTCAGACTGATATTGGATAATATGCTTTTTTACTAT
AGCGATCTTACGTCTTGTATTGAAAAGCTGACTAACACTCTGAGCGCCTACAGGTTCTAAGTCATTCTCC
AGTTGCAGGAAGTATATAATCCTITTATCTAATGCCTCCACATCTCGATACAAAGAGTTTTTTTTTTAGTG
TTAATATGTGTGCTTCAATTAATTTAACAGACCGTTGATATTTATCTTTGATTTTTAAAACCTTATCTAATT
GTTTCACCAATAAGCTCCATTGTCTGCTCATAAGTAAAACCGAGTCGATAATCTTGCTTCAGAAAACTCT
CAACAACAGATATTTTGTTGTAGATTTTATCCTGAGAGGCATTCTCCCCCGGTTTGTATTCACCAAAGTC
AATAATTGTCCTTAGCTCTTCTATTTCAGAAAGTAGCTCCCGAAATGCAGCGGCCATAATACGATGTTTT
TCATCAACAACCTGTGTAAATACCCTGCTTATACTTTTTAAGGAATCAATTGCAGGAAATTGTCCTTTTTG
GGCTAGATTTCTGCTCAAATATATATGTCCATCTAAAATGGATCTTACCTCTTCAGCTAATGGATCAGCA
AAATCATCATCCTCCAAAAGAACAGTGTAAAATGCAGTAATAGAGCCACCTGCCTTTAACTTTCCTGGCC
TTTCAAGAAGTCTGGGTAAGCTATCAAAAACCGAAACCGGATAGCCTCTTCTGGCAGGTGATTCTCCAG
CGGCTAAGGCCACATCACGTAATGCTCTGGCATACCTTGTTAATGAATCAATAAAAAGCGCTACTTTAT
GTCCTTCAGTCCTAAAAAATTCGGCTATAGCAGTGGCTATATATGCAGCATTACAACGATCAACCGAAG
AGTAATCCGAAGTTGCATATACTAAAACACACCTGTTTTTTTTCTCAGAGTTTTTCAAATAATCAACCGTT
TCTGTAACCTCTCGACCTCGCTCACCAATTAACCCAATAACATATATATCAGCACCACTATGTTCAATGA
GCATATTCATGAGAAAAGTTTTGCCACAACCAGCTGACGCAAAAATCCCCATTCGCTGTCCTTCACCACA
CGTGAGTAAAGAATCAATAACCTTAATACCTGTTAAAAAAGGCTTCTCAATTGCAGCCCTTTCACTATAT
AGCGGAGGAGCATTATCTACAGGTCGATAAAGAATTTCACTGTTATCCGTAACAGCAAACTTATCAGTA
ACCTCCCCTAAAGGATTGACTACTGCACCCAATAATCCACGGCCGACTTGCGTATGAAGAAACTGGGCA
GTGGGCTTAATCAACGTTTGCCGTGAAAGTCCACGAGAATTTCCAATCAAGCTTAATATTGTTTTTTCAT
CATGAAATCCTACAACCTGAGCTCTTGCAACAATTTCATTACTTTCAATTCCAGCCTGAATGTTACAAATC
TCACCAATCGAAACATCGCTAAGACTTGTTTCCAAGATTGGCCCCGAGATTCTATTAGGAAAAGATAAT
TGAGTGAGCAATTTTGTATAGCTCATTGATTATAAGACCTCATTTAAGATTTCCATCCTCTGATAAAACTC
ATGCAGAATCTCTGCAAAAACAATCCCATCGTGAACGTAGTCATCTTTAATAACAACTCTTAACTGTAAG
TACTCATCAGAACGATGAAGCTCTACCAGCTCATTAATGCTATAACTAAAGTTCATTAACATTAAATTTA
ATATGTTATAAGCAGATGACTGAAGTTTTACATCACTTGGTGCATCAAAATTAGCCCATAACATAACCTG
CTCATTGACAAGAGCTATATTAATAGCAGGCATACTATCCAAAGAAATGGTAATCGCTGAATGGCTGTC
TAAATCTGTAATAATTGATGGAGGACAACCAATCGTGAAAAGACTATCTCTAACTAATTGAACTAAATT
AATGTTACTCATATTTAAACCTCACTAAATAGTCTTTAATACATTAATGGTATATGCTTCATCAATCTCAG
CATACGATATAACGAGTATACTCTTAAATCTGTTATCTATAAGTCTTTTAACAAACCTACGTATATCTACT
GATACCAAAAGAACGAAATTTTTTTTTGCATTTCTCAATTCTCTCAAAGCATGTGCTAAAGTTTCCATTAC
CTCATCCGAAACCTCTATATCCATATTCAAGAAAGAGCCACCAGAGGTTTGCCTTATCCCTTTTCTTATTG
CATCCTCAATATATCCGGAAAGCATCACAACTTTAATCTCACCAGAAACAGCTATTTTACTACAAATATA
CCTAGAAAGTGATGCACGGACATGTTCAACTAATGTTATGACATCTTTTTCTCTGGGAGCCCAAAGCGC
CAAAGACTCCATAATAAGTTTTAAATTGCGAACAGAAATATTTTCTCCAAGCAATCTTTGTAATACCTCA
GAAATTCTCTGTATAGTCACATGTCGGAAGACTTCCTTTAATAGATCAGGATACCGATTTTCAAACTGAT
CTAACATATTTTTTGTTTCTTGTATACCAAAAATCTCATTTATGTTGTTTAATAAAGCTTGTGACAACTGG
TGATAAAATTCATCTTGAGCACTTTTAATTTTAGCATCAATATTAGTTAGATTTTCTGTATATGAAACGTC
TACCCAAGAAATAACACGTTCATTATATGAAGTTGAAACAACAGGAATACCTAGAGCATCTATATCTCC
ATTTTCATCTGTAATACACACTTTGTCAAAATATATATTAAAACTATCGGCACGCACCTCATTTATTAACA
AAACAATATCATCGACCTTAAGCTCATTGCTTGTTCTATATAAAATAGTTGGAAGCCTGACACCATAATC
AATAAAGAACTGACTTCTTATCCTTTCAATAAGACCTTCCATATCATTTGCATTTATCTTATTTTCGGCAA
ATAATAAAATTAAAGGAACGGTTTCTGAACTAATAGCATCCAGATTTTCTATCATTGCCAAAGATGAGT
CATGGCTATTATCAATATCAAATGTACCAGTATAGCCACTAGAATCAGACTCAGACAGACTTTTTTCTTT
TTCTACGACCTTTTTATAATAGAATAAAGCCGTCAAAGTAACTGCTATCAGGAAAAAAACAAAAAACGG
AAAACCTGGCAACATCCCTATCGCCAAGGCAAGAGCTGATGTAACAATGAGAACAAAAGGATTCCCAA
ATATCTGGGACATTATGTTACGTCCCATATTATCGCTATCACCATTAACTCTTGTAACGATAAATCCTGCA
CTTATAGAAATTAACAGAGCAGGAATCTGAGAGACCAATCCGTCACCAATAGTTAGTATGGTATAAGTA
GATAATGCACCAGATAGGGACATTCCATGTTGACTCATACCAACAGAAATACCTCCTATTAAATTGACA
AAAATAATAATGATGCCAGCAATTGCATCACCTTTAATGAACTTCATTGCTCCATCAAATGAACCATACA
ATTGACTCTCACGTTCCAGAATACTGCGTCTTTCTTTAGCTCCTGCGGCATCAATAATTCCAGCCTTTAAA
TCCGCATCAATGCTCATTTGTTTCCCCGGCATACCGTCAAGAGAAAAGCGAGCAGCTACCTCCGCAACC
CTTTCAGATCCTTTTGTTATAACAATGAACTGCACAACAGTTACTATTGAAAAAATAACAAAACCTACAG
CCAGACTATCACCAATGACAAACTGGCCAAATGTAGTAATAATTTTACCCGCATCCGCATCAACTAAAAT
TAGTCGACTAGTACTTATTGATAAAGCGAGACGAAATAATGTCGTTATCAACAAAACCGAAGGGAATG
TAGAAAAACTCAATATTCTTTCAATATAAAATGATCCCATAAAAACGAGAATAGCAAGTACAATATTAA
GCCCTATTAAAAAATCAACAAGGTATGTAGGTAATGGTATGATCAACATAGCAATGATCATAACCATAA
GCACTAATATAATTAATTCAGGCTTAGTACTTACTTGCTTAAGAAAAGACTGGATCACTTTCATCTCCTG
TTATCTAGAAAGCTCTTTCTTGTATGCACTTGTGACAAGCTCTCGTAGAATATTGATCGCAATTTCTCTTT
CACTTTCACTTTTAAATATATCATGAGGACTTTCATTAACAGAAGATAGATATGTTTGATACACTACAGA
ATCCTCATGACTGGCTCGTGATACTTCTATAACAGAAGTAAGAATTTGCTCACTTTCACTTGGATATTGA
AAAATATATAATAAAGTTAGCAATAGCTGCTCTGCACTTAAACTGCCATCTTTTAGCAAGCTTGAGGATT
CCAGTTTCTTCATAAAAATTACGTCTGAAGTTCTAATCATCTTAATTGCAATTAATTTAGATAACACAAAA
CCAAACTCATAAGCATTACAGCTCGGTTTCTCAGAATCCATGTCTACAATCAGTGACTGCTCTACAAAAT
TCACAATTGTATGTCTGTGATTAAAACCAAAATTACCAAGCCATTCTATATACTGATCTGTTGTTGATAT
GTTCCCCATGATAAAACCACGATAACATGCACGCAAAAGCTGTGGTTCAAGTTTCATTTTGTTGCCAAAT
AATTTAGCCTGTATAGCTGAATTTATTCCAGCATTAACACTTTTTTCACTACCTGATATTATCTCATTAATA
ATTTTAGTTAATGATTCGACTTGTTCAGCAGTAAGATCTTTTTCATTCAAAAGTTCCCTTAATGCTAAGAC
TTGATCACTTGGATCTTTAAAATATCTCTTTAGTCTGTCTATAAAATCCCGATCTAGAGGAAGGTTATCTT
TTAACGTTCTCTTTAAATCAAAAATCTTGTGATTAATTTCATCTTCTTCCCCATCTAAAATACGTTCCTGAC
TATCACTATTTGTTCCTTTCAGTTTCTCAAGGTCTCTTCTGTTTATAAATGACGAAAGAGCGGAAGCCATC
TCATCAGATGAGTCAATATACTTGCTATTAGCCAAGCCAGAATCAAGCTCTGCATCAGCAGTCTCATCTC
CATCATCTGAATTTGTCATTGCATTCAACTTATCAATGAATGCAGAGCTAAAAACTCCTGTATTITTAACA
TCAAGCATACATCACCTACTTTATAGTTAGTAATTTAAGTATGAAACCAATGATTTTTCGTCTTCCAGCAA
AGTAGTTTCAGATGGGATTATTTGTGTAGTCTTTTGTATTTCTCTTTCAGATATGAGGCTITTATATTCTG
CAGTATTATAATAACTTGACTCCTTTATCTCACGGGGTTGAATCAGAAAAACCCTTACAATATTACTGAT
ATTACTTGTTTTATACTTGAACACATTACCGATTACAGGAATAGAGGAGAGAAAAGGAATAGATACTAT
TTCGTTAGAGTTCGTTTCATGCGTATAACCACCTATCAACAAACTTTTGCCTTGTGGAACTCTCGCTATTG
TACTGATTTTGGTCCTTCCAACTTCTGGAAGGACAGATGTATTTTCATTATTATAATTGTAATTTGACTGA
CTATTTCCTGTTCCATCCTCTATCGTTAAAGACATCTCAATTTGTCCCCGAGATGAAAAGCGAGGTATAA
CATTTATCAATGTTCCATACGTAACATGCTCCAACGAAGAATTGCGCTCACCAACTAATGACACATAAAA
AGTCCTGTTATTATCAAATATAGCCGGAATATTCTCTTGTGTCAAAATCACCGGCCTTGAAACTACATTT
GCTTTCTTTTTCTGATTTAATGCCATAACTGAAGCAATAAACTTATTGCCGTCCAAAGTGCTTATCGATGC
TGATGAGCTCATATTAAATGACGCACCAAACGAATCACCAAATGAAGCCGTTCCTTGCCAATTAACACC
AAGGTTATTTAATTCAGATTTATCAATATCGATTATCCATAATGATAATTCTATATGTCGTTTGGCTACAT
CTAACTGAGTGATAATATCGCGAATAATCTGTATTTGCTGATCATTACCTTTGACTAATATAGAATTGGT
TTCTGGATATGCAATAAGTGATACATCCTCTAATATAGAACTATTGGTAACTGAACTAAATGAAAAGTC
ATTACTATCCTCAGATACTTTCTGTGTAATATTAAAGGGTGGCATCGGATCATTTTGGGCTTGTCTGTTA
CTCAGAGCCTTACCATTATTTAATAAACGTTCTACAACAGTAGCCACCCCTGGGATGACAATATCCTCCC
CTCTCATATTATAAGTTCTGTCACTAACAAATGTATTTTTTAATTTTATAACCCCAAAATTAACTTTATCTG
TCCCAATAGAAGAAACCTGCTTATCAAGCAAAGTTGCTGTATTAGCTACCAGTTCAACCAATGCCGGAG
GACCAGATATATAGAATGTTTTATCACTAATATTTCCTCTTATAGGATATCTGTGATCATAAAGGTTAGC
ATCCTTCAGATACTGAATAAGATAATTAAGTGATATATTCTCCAGCAAAATAACTTTGCTTATTAATTCTC
CAGAGTCATATATGTATAATGCATTTCCATCTTTATACCATATGAGTCCCACAAGTAATGTCAGTTTTTCA
AGCATTTCTTCTGGATTAGATAAATCAAACTCCCCAGATATTCTCTTTTTTGCAGCTTGTTTGCTAACAAC
AATAGGATAATTTAATAACGCAGAAAAACGTTCAAAAAAAGATCCTACCGTATCACTTTGAGCAACATA
TTTCGCTATGTCATTTTGTTCTAATAAATGAGAGTCAATATTATTAGCATTAACTATCAATGGTAACAATA
CAATCAAGAGAGTCAAAGATTTAATATTAAATTTTTTCATTTATTTTTTTCACTAAAAAAGTAATATTCGA
TAATTCTCTTGCAGAAAAGCCTAGTCGTGTTTTTATCTCATTTGAAAAATGGGATGTTGATGCATACCCA
TTATTCATGGCAGCAGAAGTGATTGTTTGATTGTGCAAAAATACATCTAATAAACCATTTACTAACCGCC
AAGTGTTCAGTTGTTCTTTGACTTTTGCCCCTAAAGCTTTTCTACATAAAGATCTAAAATAAGCCTCCGAC
ACACCATAATGCTCTGTCAGACTCTTTATTCTTACCTCCTTTTCTTTCTCAATAGTTCTCAGAAGATAAAG
AACCAAATGATATGACTCAGTCATTCGTATCATAGCCAGTGCATTACTTACATTCTGGTCAGATGAAAA
GATGAATCTTAACATTAACTTATCAAGCATCGATTCGGTATTACAACTATCCCGAAGATCATTCTCATGA
TAGAAATGAATACATTTTTCAGCATGGCTATGCTCTACTAGAGCACCACTGAAATAGTCCCAAAAAGCA
AGAAATTTTGCACAACTATTAATGTTAAAGCGATAATGATAGTTCACATTATCACTGAATTTTATATTTTG
ATTTCGGACAAAAAAAAACCCTTCCATGCTGGAGCCAGCTCGATTATGTTACGTTCTTTATCTTTTACAA
GCTCAACGTTAACTGGTTCATGTCCACTAGAGTAAATGTAAAACATATTACTTGTATTTAGACCTTTATA
TTTACTCACAATACAATTATATCCCTCGAATTAATTTTAATTTAACTTCCCCATCCTTCTCTATAAGGATGG
TATCATTTATCTTATTACCTTTTAAAACAACCCCGTCTTTCAGCATAGAAAATAGTTTATCTATAGCTATCT
TTTGTGCATCCATGAAGCACCCATCTACTTCCTTAACATTAAAGTTGCCCATGAATTTACCTGCATTAAAA
CTATAAACTGCCTTACCACTAATACATTTAGAGTAAACATTTATACTTTTATTGGTTGTATTAAACGACCA
TAATAAATCATTTGGAATAGAAAAATAATCCTTACTTACAGGAACAATATGCCATTCTTTTTCAGAATTA
GATGATGACTTTAAAGCACACCCACTCAGTGTTAATAGCAATATACTTAAAATAAATATTTTCAACTTAT
TACTACCATGTCGAATCATCTGCCTCTCTCCCTGCTTCAAAGACACTAATTAATGAATCGAAGGTTGTAG
TCTTAATATAGAAATCAATATTGTTATATATGGACAATGATGCATAAAATCCCTCATGCAAATCGACATA
TTGATTTGCATCTATGAGTTTATTAAGAAGTGAACTTTTTTGTGCAGCTGTAAGATAAGGAAAACGTGA
ATTGAATAATTGTTCAATCTTATTGTGCAGTTCATCATTATCTATGGAATTTAATTCACTCTGTGAGAAAT
ATTGCATTATTTCATAGATTATATCCTCATCAAATGATTGCGAAGATGATAATCTCTCATTTAAATTAACA
AATTCTTCACTATTTAATCTTTGCTGCAGAGCATTGCTTGCATTTATTTGATTAATCATTAACAGGATTCT
CTTTTAACAAGTCTTCTGCAATTTGTCTCAGTTTCTTATGTGCTAACTCTTTGAAATATTTTTTATCATTTTT
TGTTAATTTTTCCGCAAGTTCTATTTTAACACCTGATATTACATCCTGAGGACTAAACTCCACTACTTGAT
TACCACTACAGAATATATAATTATTCCCTTCAGAATATTTTAAAATAATCTTGACATCTGAGCGATATTTA
TGGAGATCAAGTTCTAACTTTTTCCTCAGTGCTAAATTACACTTGGGCAAAACCACTTTTAATTCTGTTAC
TGTATTACGCAACGAACTAAGCCACTGCTCCAATAAAGTGCTAATGATCCTCTCATCATGTAAATTTTCT
GAAAGCAAACTAGTTATATAGTCCTCAATATTTCTTCTGTTTTCATTTTGCTTTAACTTCCATTCACAAATA
TAGTCAATAATATGCTCAAAAGCAGTCTGTATACCAATTTGATAACCATCACATACAGCATCTATACGCA
GTGATTCAGCTTTTTTCGTAGCATCCTTTATAACACAAATTGCTTTCTTCTTACTCTCAGTGTCTCTATCTT
TTATGGTTTTACATAATTCCTTTTCAATTCTCTTTATAACAACACCATCATAGGCATGATGTCTGGATACC
GGTAAAGTGCCTTTTTGCATATTGCATACCTTCATTTAATCGGGACAAATTTCTTTCTACAGGCGTATAAT
TTCCCTCCAAAGCAATTGGATTAAATAATAAGTTTATTCTTTCATATATACCAACAGATATATTTTCCTTC
CACGCAATAAGATGCCAAAAACCACTTCGAATAGCGGCCTCCCCTTGCTTACTGATTTTGCCCCCATAAT
AACTAATAACCATCTCACTATGATTAATTAACCGAGACTCTTGACTACCGATAAAATAAGCAACAGCTG
GAAGTAAATTCCAGTTGGCAACAAACATAGCGGTTACCCCGCTTAAAGGTTCAATATCGCAATTTAGAT
CATATTTATTAATTATGAGATTATTAATCTCACTTCTGATTACGCCATTTTCTAACAGTTCAGAAGGCAAA
TTCAACCGATTTCTATTTATATAGAACGCTGGATCAAAAATTATTGAAAGATATTTTTTATAAATTCCATC
CATTCTTATCATATTTTGTTTCTCTTGAACCACCCTGTTTTGAATGCCCATACCAAAAGAATGACAACTAG
TACAGCCATCCCGAGAAATAAATATATTACTTCATTTGTTAAAAATTCCGATTTAATTTCCTTAACAGGTT
GTACATTTGTATAAACATATTCTTCTTTCGGAGTTAATATGACAGATATATTTTCATACTTAACATCAGAA
AAGGTGTTTTTCAAAAATCGCTTAATATTACTAACTAATAGTTCAGACTCTTTCGGTGAGTCATATATAG
CGATTACTGATATATGCATCGGTTTTGAAGATATATTTTTTTCTTCAAGATCATAGCTAACATGTATTTTT
GCCGAAATAACACCACCAATAGAAACTAAAGACTGTTCCAGCCGTTGCTCAATAGCACTATATAAACGG
GCCTTTTCAGCTCTTGGAGAAGACACTAATGAATCTGTAGGAAACATTTGTGAGATATCAACTCTCTCCG
GATTTGGCAAATCGTACATGCGCATCAAATCAACTGCCGATGCAAATGTCCCCTTTTCGACTTGTACCGA
GATCCCCTGTTTACCTCCATCAACTTTTCTAGCAGTAATATTATGGCGTTCTAGCACAGATATTATTTCAT
TTGCCTGTCTTTGAGATAAATTAGAAATTAACTCTTCACGTTGCTCACATCCAATCAACATCAGCAACAA
GAATAAAATAAAACCTTTATACCTAATCATATAAATTAAGACTTTAATAAAGTTTCAACAGCCGAAACTG
TTTTTCTTGCTAACGTGCCAGCTAATGATACTCCTATTGAATAATTTGAGAGCGTCGTCTGTAACTTGGC
CAAAGATTCTGGGTTTAATGATTCTGGATTGCTTACCATCTCCATTATTTGTGATACTTGAATATCTGTAT
CCATCTTAATATCACTATATTTAGCCGACACGACGTCTTCCAGACTTGATATCTCTTGAGATTGAAAATC
ACTGGCTTTGATAATATCAACCTGATTGACTGGATAAATGTAATTCATGAATGCTCCCTATTATCTGAAG
TTTTGAATAATTGCAGCATCAACATCCTTAATCACTTTCACTGTATTGGATTGCGCGTTCCTATATAATGT
ATATTCAGATAATTTACTTTGGTATTCAGCCAGCAACTGTGGATTCGAAGGATTTTTAGCTAATTTATCT
AGTGCCAATGTTAGTTCACCTTGTAATGTTTGAGTTCCATCATCAAAAGTTTCAGATAATGAACTCAGTG
TCCAATCATCATTCGGTACTGTAACACTCATTTTATCCTCACTTATTITTATCTAAAAAAAACCAGTGTTTA
TCATTCAACATTACATAACTGTCTTTGCTGTTAAGATATGATTTACCTTTAAAATCATCATCTATCAAAAG
TACAGAAAACTCAATATACTGATCTCCATATATATTTTTATGTTCAGAAATCAGTCTATTAATGAGCTCAA
GCTTTTCATCTGTCAGTTCTTCTCTGACAGAATATGTAACTTTATTATTTTCACAAATCTCTTTATACTGGA
CATTTACCTTTGTGAAAATACCTTTAGCATCCCCCCTTGCATTATGATCGCTAAGATAGTTGAATTTAATA
TTTTTGATATACGGAAATTCAACAATCAGAGCATTTTTCAATTTATCAAGTTCGGTATCTTTTGAACTATT
TCTTTCTTTACTTAAAAACACCAGTAATTCCGCTTTATCACTCACTAAATTCAACACATACAAATTGATAA
ATGGGAATCGTTGATTGATATATTGTTGAATTCTATTTATTTCTTTGTTTGACACCAGAATATATCTGCAT
TTGGTAAATCCGGTTTTATTTAAAGCTACAGATGCCCATACCGCATCATTTTGTGTTTCGGCAAGAATAT
AATTGCACTGTCCAGTGTTGACTATTCCATATCTCTTATCATCTATAATTTCAGCAATTTCTITTACTTCAT
CTTTCTTTAAAGAGAGGGAAAAAATTATAATTAACACAACAATAACAGCAAAGAAAAAAAACACCGAG
TGGTTCTTATACATTCCGTTTAGTATATGGCCTCTCGATTTATCCTCCCTCATATTTTTAAGGTGAAAAGA
AATCCCGTCATATTCAAATGAATTAAATTGTGAATTGATATCAATTTGCTCACCTTTTTCATCACTAAATT
GACAGATATTATCTTTGCTTATGGTCAAATAAATTATACCATCCGATTTACTATCTGTTACAGGAATAAT
ATTTTCTGGGAAATTGTCGTTTCCAAACTCAAGTGTTTCTATCGTCCGTCCGAGAACAATTAAGTTATTCC
CATAATATAACGGAAACTCATCCCCGACACCGTTTGTGAGTTTCACTAATAACCTGAATGGTGCAAGAT
TTGAGTTCTTTGCCTCAGACATAAATCCTCTTATATCCTTCGATTATTCTGCTTGCTCTCTGCTGCTATCCG
CAAATAGCGTCTATAGATATCCTCTGCGTACTTTAATCTTTCTTTTTTCTTTTTCGGAGACGTTCCTGCATT
ATAGGCTCCAACAGCCTCCCACCCTCTTCCATACATCATCATAAACTCATTTAATAACTTTGCTGCATAAT
GTACAGAAAGACATGGATGACTAATTAGCATTTCCTCAGAATATCCCATTTCTCTAAGTCTTTTAGAATG
AAAGTCATTTATTTGCATTATACCATAATCTTTACTTCCATTGTTATTAACATTGACTGCAAATTTATTAAA
TCCGGATTCTTTTTCCGCAATCGCTTTTAACAAACTTGATGGAATATTGTACCTTTCACCAGCCTTATCCC
AACAATCAGCTCTCCCTAAATATGGGATAAAACACAGCAAAATAAATACGAAACGGGACATTAATACCC
CTTCATTCTTCGCGCAAATTCATCCTTCAGCCAACGCACACGCGAAATATAATATTCTGTATTAACATATA
CATCCTCGAATTCATTTATCCCGGTAAGATCTAATCGCGCAATTAGATTTCCGCCTTCATCATCAGTAGCT
AAGCATATCTTTTCTGAGTAATTTAGGCTCAACGCATATATAAGATCATTTATATTTTCAGGCAATGCAC
AAAAAGGGCATAATAAATTTATTGAATCTCCTTCTTTTTCAATATATATTAGCACATCATCATCAAGCATG
ATTGCTTGATCATCGATATCAATTAAAGGGATACCCAAGGCTCGGCAAATAACATCTGCTAAATCTTCCA
TATATTCCTCTTATACAAATGACGAATACCCTTTCACCATATTCCATATTTTTGAGTCCCCTATTCTTTCAG
AATAAGATAGCTCTAACGAAGATAGCGGCAATTTTTTCATGACTTTATTTCCGGGCACACCAGTATTCAT
CTCTTGGATTTCCATATTACCACTATTCATTAGAATAGTAGAAAATAATCTTTTCTCTTCTGAGGATAATT
TACTATTTAATTGAGAAAACTGACCTGTTTCATGTTTTCTTATTATTTCTCTTTTAATTTCAGCATCCTGCA
TACCTGTTCTGTCCTTCCCACTCTTACAATTCCAGCAAGGTACAGCACCTATAGTATAAGCCAATAGAGT
CATTCTTTGTGACAATTTATACGGCTCTCCATTATCATTTTTTTGTAACTTCTTGTTTATAATCTCTTTTATC
TGGTTAGCCAGATATATCACATCATTCTTACATGGTGGATTTTTTTCTATTGCTTCTGCAGCCCAACCTCC
AATCACACCATTTTTGAGGAAATTATCCCCCAACAAAGAACATATTGATTCATCATTAAGCTTGTCAACA
TTCCTCCAGCCTAAGCCCATTTTTAGCGCTAATTCATTTACACCAAAGTTAAATGTCACCACTTTTAGATT
AACGCTTACTTCTTTCAGAAGACCATCACTATTCCGAATTAATAATTTCGTTGGCTCCCCCCTTTTAGAGT
TTAACCCTTTTAATGCGTTCACCTGATCTTTTAACATACTCTCCTCTCCCCCGGTTAAACTGGTCGGCGTC
AGAAGAGAAGTCGAAACAATCTTTAAATCTACTGTTTTACCAGACAAAGCCTGTGATAATAACTCTGGT
CTGCTATATAATGCTGCGCTTACTAACTCCTCAGCTTTATTACGAGCAGCAACAGCTCTTTCAGATGAGT
TTTTTTTCAATCCATAGGCTGATATAACTCCATGTCTGATACCCGAAAAAATTTCCTTCCCCTCGTCATCC
ACCACTTTTGAAAGCCACATATTCGCGATATGATCACTCTCTCTCGTAGAAGCACAACAAATTCCTTTTCC
ATTATACTCTTTGACAAATATATTTTTATTCCCTATCTTCATATGAGAGGCAGGTGTTAATTGAAAACCAT
AATTTTTCCCATGATGACTAATATTCTTGTTGACCGGTCCCCAATTCTTATTATTTAAATAATCAGTAAAT
ATTTGTTTTATTTTCGTTTGCAATTCCTTTTTATTTTTAATGCCTAGCTGTGCTCTTATCTCTTCACTTATTA
CTTTATTAAGCTCATTAATATGTCTTTTATTAACTTCACCAAGTTTATAGGTATCTAATCGTTCCATCATG
GCTTTAATTTTTGACGTATCACCACCTGCCAACTTTATTGCCTCGTAAAAAACTTCAGCTCTGTCACGCGC
AAACAAAAGGCTTTTTCCTGTTAAGCTTGTTTGGTTCTGTAAATATAATCGATTAAGCGCTTCGATCCCC
TTATTTCTTTCTCCATTTTTAACTCTTTGATATGAAATCACGCTTACACCTTTGTGCTTTCCGGTTTCTTCTG
CGCCTTTATAGGAATCTCCGCTTTGAAATGAAACCTGATGCAATCCCAAATTAGTTATGTGCATTTAATT
ATCCTCAGTCATAAATGGGGCATTGATTGCAGTTTTGTACCTCGTITTACAACAAAAAAGATACTTATTC
AACAGGGTTCTCTCTTTTTCTATAACACTCCGAGCCTGATATATCAGACACTTTAACTTGTGGGATTTCAT
GATGAAACGAGCACTACATATGCTCACGAGGTACATATTATGTACAACAAAAGAAAATTTGATTATGTT
AACAGGCTCAAGTTTCTAAATTTATTAATTGAATATATTATATATCAAATACATTTTACATCCCCACAATC
ACCACGTAATGGAGAGTTAATAAAGTATGATCCTCAAAATTAGCAATTTCATTGACGCAAGCAATACAA
AAGGGCCTATACGCGTTGAAGATACAGAGCATGGACCTATATTGGTAGCACAGAAATTCAACCTAAAG
GATCTCTTTTTCAGAACATTAAGCACTATTAATGCAAAGATAAATTCACAGATATTAAATGAACAACTAA
AAAATTATCGACTAGCGAATCAAAAATCCTTATTGCTTTTCCTCAAGACATTAGCTAGCGAAAAATCTGC
AGAAAGTGCGTTTGCCGCCTATGAAGCAGTCAAAAATTCAATTCAACATTCTTTTACAGGTAAAGACAT
CAAATTAATGTTGAATACCGCAGAGCGTTTTCATGGCATCGGTACAGCCAAAAATCTTGAAAGACATTT
AGTTTTTCGTTGCTGGGGAAATAGAGGCATAACCCATTTAGGGCATACCAGTATCAGTATAAAAAACAA
TTTACTACAGGAACCAACTCATACCTATCTCAGCTGGTATCCCGGCGGGAATGTTACAAAAGATACAGA
AATAAACTACCTTTTTGAAAAACGCTCAGGTTACAGTGTCGATACCTATAAACAAGACAAACTAAATAT
GATTTCAGACCAAACAGCCGAAAGACTTGATGCAGGCCAAGAGGTTAGAAACTTATTAAATTCAAAAC
AAGATCAAAATAACAACAAAAAAATATTTTTTCCCAGAGCTAATCAAAAAAAAGACCCCTATGGTTATT
GGGGGGTCAGTGCTGATAAAGTATACATTCCATTATCAGGTGATAATAAAACAAAGGATGGTAAAATC
AGCTATAACCTTTTTGGCCTTGATGAAACAAACATGTCAAAATTTATATGCCAAAAGAAAGCAGATGCT
TTCAGACAATTGGCAAATTATAAATTAATCAGTAAATCTGAAAACTGTGCTGGTATGGCACTTAATGTTC
TTAAAGCAGGAAATTCTGAAATATACTTTCCACTCCCTGACGTTAAATTGGTTGCTACACCTAACAATGT
ATACGCGTATGCAAATAAAGTTAGGCAACGCATTGAAAGTCTCAATCAGTCTTATAACGAGATAATGAA
GTATATTGAATCTGATTTTGATCTTTCCAGATTGACTCAATTACGACGTAGCTATCTTAAAAGTTTCAATA
AAATTAACCTTATCCACACCCCAAAAACATTCAAGCCTTTATCAATTTCACTTTACAAACATCCTACTGAA
AATGTATCTTCAGAAGACTTTGATGCGGTCATCAACGCTTGCCACTCATACTTAGTAAAATCAGCACCCA
GCAATATGTCTCGTGTATTAAACGAATTAAAAACCGGAGCAACAGATAAAAAAGAAGAAATTATCGAA
AAGTCAATAAAAACTATTGATTATTACAATTCTCTTAAATCGCCAGATCTTGGAACCAAACTTTACATTC
ACGACTTATTACAGGTCAATAAGCTTTTATTGAATAACTCTCATTCTAATATATAGAAGGCCATAGAAAT
GTGTCGCAAACTATATGATAAACTTTATGAAATAACAGGTGCAAAGCTTGATTTTAACGATAAAAATCA
AGCCTTTATATTGCTCGAAGAACAAATACCTGTTTGTATAACAGATAATGATGAATACATCTTTTTGACC
GGACTTCTTAATGAACATGAACTTTTTACAGAAAATATTATTAATCCAGAACACATACTCATCCTTAACT
ACTCTTTGAGTAGAGATTATGGTTCTTCTATCTGTCTTCTCCCTGATACTCATCAATGTGTTTTGACCAAG
AAACACTACAAAAAATATCTTTCACCTGATGAACTCATTGAATCTTTGTATGAATTTTTATTTTGTATTAA
ACTAACCATTGCAAACATCACTTCAGAAGTGAACTAACATATAGGGGGTATCATGCAAATTCTAAACAA
AATACTTCCACAGGTTGAATTCGCGATTCCTCGCCCATCATTTAATTCGTTAAGTTACAATAAGCTTGTA
AAGAAAATATTATCTGTTTTTAACTTAAAACAACGCTTTCCTCAGAAGAATTTTGGATGCCCGGTGAATA
TAAATAAAATCAGGGACAACGTCATAGACAAAATAAAAGATAGTAATTCGGGGAATCAACTATTTTGTT
GGATGAGCCAAGAGCGAACCTCTTATGTCTCTTCAATGATAAACAGAAGTATAGATGAAATGGCCATA
CACAATGGAGTTGTTTTGACCTCAGATAACAAAAAAAACATATTTGCAGCTATTGAAAAAAAATTCCCG
GATATAAAACTTGATGAAAAATCAGCACAAACATCTATCAGTCATACAGCACTAAACGAGATTGCCTCA
TCTGGCCTCAGAGCAAAGATTTTAAAACGCTATAGCAGTAACATGGATTTGTTTAACACACAAATGAAG
GATCTTACAAATCTAGTATCCAGTTCCGTCTACGATAAAATATTTAATGAATCAACAAAAGTTTTACAAA
TAGAAATAAGCGCAGAGGTTCTAAAGGCCGTATACCGTCAAAGCAATACAAATTAAATCTGTTTATATT
AAATCTTATACTTATTAAAGTAAAGCAGACAAAAAGGAGACCTTATGTCTTTAAATATCACTGAAAATG
AAAGCATCTCTACTGCAGTAATTGATGCAATTAACTCTGGCGCTACACTGAAAGATATTAATGCAATTCC
TGATGATATGATGGATGACATTTATTCATATGCTTATGACTTTTACAACAAAGGAAGAATAGAGGAAGC
TGAAGTTTTCTTCAGATTTTTATGTATATACGACTTTTACAATGTAGACTACATTATGGGACTCGCAGCTA
TTTATCAGATAAAAGAACAGTTCCAACAAGCAGCAGACCTTTATGCTGTCGCTTTTGCATTAGGAAAAA
ATGACTATACACCGGTATTCCATACTGGACAATGCCAGCTTCGGTTGAAAGCCCCCTTGAAAGCCAAAG
AGTGCTTCGAACTCGTAATTCAACACAGCAATGATGAAAAATTAAAAATAAAAGCACAATCATACTTGG
ACGCAATTCAGGATATCAAGGAGTAATTATTATGCATAATGTAAGTACCACAACCACTGGTTTGTCTCTT
GCCAAAATATTGGCTTCCACTGAGCTTGGAGACAATACTATCCAGGCTGCAAATGATGCAGCTAACAAA
TTATTTTCTCTTACAATTGCTGATCTTACTGCTAACAAAAATATTAATACAACTAATTCACACTCAACTTCA
AATATATTAATCCCTGAACTTAAAGCACCAAAGTCATTAAATGCAAGTTCCCAACTAACGCTTTTAATTG
GAAACCTTATTCAAATACTCGGTGAAAAATCTTTAACTGCATTAACAAATAAAATTACTGCTTGGAAGTC
TCAGCAACAGGCAAGACAGCAAAAAAACCTAGAATTCTCCGATAAAATTAATACTCTTCTATCTGAAAC
TGAAGGACTAACCAGAGACTATGAAAAACAAATTAATAAACTAAAAAACGCAGATTCTAAAATAAAAG
ACCTAGAAAATAAAATTAACCAAATTCAAACAAGATTATCCGAACTCGACCCAGATTCACCAGAAAAGA
AAAAATTAAGCCGGGAAGAAATACAACTCACTATCAAAAAAGACGCAGCAGTTAAAGACAGGACATTG
ATTGAGCAGAAAACCCTGTCAATTCATAGCAAACTTACAGATAAATCAATGCAACTCGAAAAAGAAATA
GACTCTTTTTCTGCATTTTCAAACACAGCATCTGCTGAACAGCTATCAACCCAGCAGAAATCATTAACCG
GACTTGCCAGTGTTACTCAATTGATGGCAACCTTTATTCAACTAGTTGGAAAAAATAATGAAGAATCTTT
AAAAAATGATCTGGCTCTATTCCAGTCTCTCCAAGAATCAAGAAAAACTGAAATGGAGAGAAAATCTGA
TGAGTATGCTGCTGAAGTACGTAAAGCAGAAGAACTCAACAGAGTAATGGGTTGTGTTGGGAAAATAC
TTGGGGCACTTTTAACTATCGTTAGTGTTGTTGCAGCAGCTTTTTCTGGAGGAGCCTCTCTAGCACTGGC
AGCTGTTGGTTTAGCTCTTATGGTTACGGATGCTATAGTACAAGCAGCGACCGGCAATTCCTTCATGGA
ACAAGCCCTGAATCCGATCATGAAAGCAGTCATTGAACCCTTAATCAAACTCCTTTCAGATGCATTTACA
AAAATGCTCGAAGGCTTGGGCGTCGACTCGAAAAAAGCCAAAATGATTGGCTCTATTCTGGGGGCAAT
CGCAGGCGCTCTTGTCCTGGTTGCAGCAGTAGTTCTCGTAGCCACTGTTGGTAAACAGGCAGCAGCAA
AACTTGCAGAGAATATTGGCAAAATAATAGGTAAAACCCTCACAGACCTTATACCAAAGTTTCTCAAGA
ATTTTTCTTCTCAACTGGACGATTTAATCACTAATGCTGTTGCCAGATTAAATAAATTTCTTGGTGCAGCG
GGTGATGAAGTAATATCCAAACAAATTATTTCCACCCATTTAAACCAAGCAGTTTTATTAGGAGAAAGT
GTTAACTCTGCCACACAAGCGGGAGGAAGTGTCGCTTCTGCTGTTTTCCAGAACAGCGCGTCGACAAAT
CTAGCAGACCTGACATTATCGAAATATCAAGTTGAACAACTGTCAAAATATATCAGTGAAGCAATAGAA
AAATTCGGCCAATTGCAGGAAGTAATTGCAGATCTATTAGCCTCAATGTCCAACTCTCAGGCTAATAGA
ACTGATGTTGCAAAAGCAATTTTGCAACAAACTACTGCTTGATACAAATAAGGAGAATGTTATGGAAAT
TCAAAACACAAAATCAACCCAGATTTTATATACAGATATATCCACAAAACAAACTCAAAGTTCTTCCGAA
ACACAAAAATCACAAAATTATCAGCAGATTGCAGCGCATATTCCACTTAATGTCGGTAAAAATCCCGTA
TTAACAACCACATTAAATGATGATCAACTITTAAAGTTATCAGAGCAGGTTCAGCATGATTCAGAAATCA
TTGCTCGCCTTACTGACAAAAAGATGAAAGATCTTTCAGAGATGAGTCACACCCTTACTCCAGAGAACA
CTCTGGATATTTCCAGTCTTTCTTCTAATGCTGTTTCTTTAATTATTAGTGTAGCCGTTCTACTTTCTGCTC
TCCGCACTGCAGAAACTAAATTGGGCTCTCAATTGTCATTGATTGCGTTCGATGCTACAAAATCAGCTGC
AGAGAACATTGTTCGGCAAGGCCTGGCAGCCCTATCATCAAGCATTACTGGAGCAGTCACACAAGTAG
GTATAACGGGTATCGGTGCCAAAAAAACGCATTCAGGGATTAGCGACCAAAAAGGAGCCTTAAGAAA
GAACCTTGCCACTGCTCAATCTCTTGAAAAAGAGCTTGCAGGTTCTAAATTAGGGTTAAATAAACAAAT
AGATACAAATATCACCTCACCACAAACTAACTCTAGCACAAAATTTTTAGGTAAAAATAAACTGGCGCC
AGATAATATATCCCTGTCAACTGAACATAAAACTTCTCTTAGTTCTCCCGATATTTCTTTGCAGGATAAAA
TTGACACCCAGAGAAGAACTTACGAGCTCAATACCCTTTCTGCGCAGCAAAAACAAAATATTGGCCGTG
CAACAATGGACACATCAGCCGTTGCTGGTAATATATCCACATCAGGAGGGCGTTATGCATCTGCTCTTG
AAGAAGAAGAACAACTAATCAGTCAGGCCAGCAGTAAACAAGCAGAGGAAGCATCCCAAGTATCTAA
AGAAGCATCCCAAGCGACAAATCAATTAATACAAAAATTATTGAATATAATTGACAGCATCACCCAATC
AAGGAATTCGACAGCCAGTCAGATTGCTGGTAACATTCGAGCTTAATATATCCAAGAGCCATAATAATA
TATGGCTCTTCCTGTAAGGAAATAACCATGAATATAACAACTCTGACTAATAGTATTTCCACCTCATCAT
TCAGTCCAAACAATACCAACGGTTCATCAACCGAAACAGTTAATTCTGATATAAAAACAACGACCAGTT
CTCATCCTGTAAGTTCCCTTACTATGCTCAACGACACCCTTCATAATATCAGAACAACAAATCAGGCATT
AAAGAAAGAGCTTTCACAAAAAACGTTGACTAAAACATCGCTAGAAGAAATAGCATTACATTCATCTCA
GATTAGCATGGATGTAAATAAATCCGCTCAACTATTGGATATTCTTTCCAGGAACGAATATCCAATTAAT
AAAGACGCAAGAGAATTATTACATTCAGCCCCGAAAGAAGCCGAGCTTGATGGAGATCAAATGATATC
TCATAGAGAACTGTGGGCTAAAATTGCAAACTCCATCAATGATATTAATGAACAGTATCTGAAAGTATA
TGAACATGCCGTTAGTTCATATACTCAAATGTATCAAGATTTTAGCGCTGTTCTTTCCAGTCTTGCCGGC
TGGATCTCTCCCGGAGGTAACGACGGAAACTCCGTGAAATTACAAGTCAACTCGCTTAAAAAAGCATTG
GAAGAACTCAAGAAAAAATATGAAGATAAACCGCTATATCCAGCAACTAATACTGTTAGTCAGAAAGA
AGCAGATAAATGGCTTACAGAATTAGGTGGAACAATCGGCAAGGTATCTAAAAAAAACGGGGGATAT
GTTGTCAATATAAACATGACCCCAATAGACAATATGTTAAAAAGCTTAAATAATCTAGGTGGAAATGGC
GAGGTTGTGCTAGATAATGCAAAATATCAGGCATGGAATGCCGGATTCTCTGCCGAAGATGAAACAAT
GAAAAATAATCTTCAAACTTTAGTTCAAAAATACAGTAATGCCAATAGTATTTTTGATAATTTAGTAAAG
GTTTTGAGTAGTACAATAAGCTCATGTACAGATACAGATAAACTTTTTCTCCATTTCTGAGGTGCGCCAT
GCATAATGTAAATAATACTCAAGCGCCAACATTCTTATATAAGGCAACTTCACCATCATCAACAGAATAC
AGCGAGTTAAAAAGCAAAATATCCGATATCCATAGTTCGCAAACTTCTCTAAAAACACCAGCATCAGTG
TCTGAAAAAGAAAGCTTTGCAACGTCTTTTAATCAGAAATGTCTTGATTTTTTATTTTCTTCCTCAGGGAA
AGAAGATGTGTTAAGAAGCATTTATTCCAACTCAATGAATGCGTATGCCAAAAGCGAGATTCTCGAATT
TTCAAATGTTTTGTACTCCTTAGTACATCAAAATGGTCTTAATTTTGAAAACGAAAAGGGACTTCAAAAA
ATTGTCGCACAGTATTCGGAACTAATTATAAAAGATAAATTATCCCAAGATTCTGCCTTTGGACCATGGT
CGGCAAAGAATAAGAAACTCCATCAATTACGACAAAACATTGAGCACAGACTTGCACTATTAGCACAAC
AACACACATCTGGTGAAGCTTTATCATTGGGACAAAAACTCCTCAATACTGAAGTATCATCATTTATCAA
GAATAATATTCTTGCTGAATTAAAGTTAAGTAATGAAACTGTTTCATCTCTCAAACTAGATGATTTAGTT
GACGCACAGGCAAAACTTGCCTTTGATAGTTTGCGCAATCAACGTAAAAATACTATTGATAGTAAAGGA
TTTGGTATAGGTAAACTGTCAAGAGACTTAAATACAGTAGCCGTGTTTCCTGAGCTGTTGAGAAAAGTC
CTTAATGATATTTTAGAAGATATAAAAGATTCGCATCCTATCCAAGATGGCCTCCCTACACCTCCCGAAG
ATATGCCAGATGGCGGACCAACCCCCGGAGCCAATGAGAAAACATCCCAACCTGTAATTCACTATCATA
TAAATAATGATAATAGAACTTACGATAATAGAGTTTTTGACAACAGAGTATATGACAATAGCTATCACG
AGAACCCAGAAAATGATGCACAGTCTCCTACTTCTCAGACAAACGATCTATTATCCCGTAACGGAAACT
CATTACTAAATCCACAAAGAGCACTAGTTCAAAAAGTAACTTCCGTTCTACCACACTCTATATCAGATAC
TGTCCAGACATTTGCAAATAATTCAGCTTTAGAAAAGGTTTTCAACCATACTCCAGATAATTCGGATGGA
ATAGGTTCAGACCTGTTAACTACGAGTAGTCAAGAAAGATCTGCAAATAACTCTCTTTCTCGGGGACAC
AGGCCTCTGAACATACAGAACTCTTCAACCACCCCCCCTCTCCACCCGGAAGGAGTGACAAGCAGTAAT
GATAACTCATCAGATACAACTAAAAGTAGCGCTTCTCTTTCTCATAGAGTAGCTTCGCAAATCAATAAAT
TCAACTCAAACACTGATTCAAAAGTACTTCAGACTGATTTTTTCTCAAGAAATGGAGACACATATTTAAC
ACGGGAAACGATATTTGAAGCGTCAAAAAAAGTAACAAACTCCCTAAGTAATCTTATATCTCTCATTGG
AACTAAATCAGGAACACAAGAACGAGAGTTACAGGAAAAATCAAAGGACATTACAAAATCCACAACAG
AACACAGAATAAACAACAAATTAAAAGTTACAGATGCAAATACAATAAACTACGTAACAGAAACCAAC
GCAGATACAATTGATAAAAATCATGCGATCTATGAAAAGGCAAAAGAAGTATCTAGCGCCCTCAGCAA
GGTATTGTCAAAAATTGACGATACCTCTGCAGAATTACTTACAGATGATATATCTGATTTAAAAAATAAC
AATGATATTACAGCTGAAAACAATAATATATATAAAGCAGCAAAAGATGTAACCACTTCCCTATCAAAA
GTATTAAAGAATATCAATAAGGATTAAAATATGATAAAAGAAAAAATATTATCAATAGTGGCCTTTTGC
TATGGTATAGCGTACAGCAAACTATCTGAAGAAACTAAATTTATTGAAGATCTTTCTGCCGATTCTCTTT
CTCTGATTGAAATGCTGGATATGATTAGTTTTGAGTTCAATCTACGTATAGATGAATCTGCATTAGAACA
CATTATTACTATTGGAGATCTTATTTCTGTAGTCAAAAATAGTACAAAATCAATATAGTAATTGATAAGC
ATTTTTTCATCTATGGAGCTCTCACATCAGAGCTCCACAAGAATATTATTCTTTTATCCAATAAAGATAAA
TTGCATCAATCCAGCTATTAAAATAGTAATTTTTAAGACTACCGTTGACTATCATCAACAGTTACATACA
ATCTTGCTCACACTGCATTTAACTTTTGTCAATATAAATCAATATGAATAAACAGGGTGTGATATGGTGG
ATTTGTGCAACGACTTGTTAAGTATAAAGGAAGGCCAAAAGAAAGAGTTTACACTCCATTCTGGTAATA
AAGTTTCCTTTATCAAAGCCAAGATTCCTCATAAAAGGATCCAAGATTTAACCTTCGTCAACCAAAAAAC
GAATGTACGCGATCAAGAATCCCTAACAGAAGAATCATTAGCCGATATCATAAAAACTATAAAGCTACA
ACAATTCTTCCCTGTAATAGGAAGGGAGATTGATGGTAGAATTGAAATTCTGGATGGCACTCGTAGAA
GAGCATCTGCAATATATGCAGGAGCAGATCTTGAAGTTCTATATTCAAAAGAATATATATCTACTCTTGA
TGCCAGAAAACTAGCAAACGATATACAAACAGCAAAAGAGCATAGCATCCGAGAACTTGGTATCGGTC
TTAATTTTCTGAAAGTATCAGGGATGTCCTATAAAGACATAGCCAAAAAAGAGAATCTGTCTCGCGCGA
AAGTCACTCGTGCCTTTCAGGCAGCAAGCGTTCCACAGGAAATAATATCTCTATTTCCAATCGCGTCAG
AACTTAACTTTAATGACTACAAGATATTATTCAATTATTATAAAGGACTTGAAAAGGCTAATGAATCTCT
TAGTTCTACACTACCAATATTAAAGGAAGAAATAAAAGACCTTGATACAAATTTGCCCCCGGACATATA
TAAAAAAGAAATTTTAAACATCATAAAGAAAAGCAAAAACAGAAAGCAAAACCCTTCGCTAAAAGTTG
ACTCGTTATTTATTTCTAAAGACAAACGGACTTACATAAAAAGAAAAGAGAATAAAACAAACAGAACTC
TAATATTTACATTATCTAAAATAAACAAAACAGTTCAGAGAGAAATAGATGAAGCTATTCGGGATATAA
TTTCTCGCCATCTATCGTCTTCATAAACATCGAACACTGATGTAAACTGCCCAATTCAGATAATAAAAAT
CTTCGTTGAACTTGACTTTCAGCCGGTGGGGTAATTCCGTCGGCTATCTGTTATTCAATCCCCACGCACT
GTGTTTCCTTAAAATTACGGTGAAAACTCATACCCTGCTGACCACGAGAACATTATCTGGTGAAATTAGT
AATATTCATATCATTGAAAATGAATTGTCAACTAAGGGATGCGGCATGATGGTCAAAATTTTATGTGAA
GATGAGTGACTGGCCGAAAATATTCAGCGTATTTTTAATTGTTTCAATAAAAACAGCGAGGATTAGCAG
TCAACGCTTTTACTACTCAATTTTTTTACATGGTGATAAACAAAAAATCCACCATTAACAGACTTCGTTCA
CAGTATCAAAAAAGCTTTAAGAATATATTAGCAGATTTCTCAAATATAATTACTGTTTTATTCTCCTAATC
TTCCCATAAAATGACCGAATTACATTATTTTATAATTATAAACAAGTTTGATTTTAAAATCATATTAATTA
TTAACAAGAGATTTATTTTTCTAACCCCAAAAGTTATGCTTAGTCAAAGAGTAACTTATAATATCCATAC
CCCTCATAGGGCTTACAGTTTAAATTGCGGGTATAAAATACATCTATTTTCAAATAAAATAAAGCATACT
CCAATTACAACGAGTGACTAAAATAAAAATATCATCCAAGTCAATCACTGTTTGCTCTGCAAAATATGTC
AGCCTTCTCAAACTTACCAGCACTTAAATATTTAAGCACACAATCATTCCTTGAGGGAGTTCACTTATTTC
AAGAGAAACATTTCGTCTTCCCACACAACTAATCCGCCCAATATAGCTAAATTTGTACTCAGTAATATCA
TTTGTTATGCCCCCCGCCCCCAAAATATTCAAGCTCCTTCACAGATAAAAAACGACAATTTACAAAGCCT
CATTAGTTATAACTATGGAAATACCTATTTTAGTATATCCTATAAAGTTAGAATTACTTCTTCTTGCACCT
AATTCCATAGTTGAAAAACAAGAATAATTTTCTCCTACAGCTGGATCCATATAGCTTCCATCAGGTCTAC
ACAACACCCAGTGGAGGCCGACAGGAACTCCAATAAAGGATACAGCAACAGCTTCCAAAACTCTTTGG
TTAGAAGATAACACATCTCGTTGATGTACTATATTACATCCCATGCCAATTAATAAGTCCCTTGCATCGG
GATAAATAAAACTAATTACTTGCGAAAAAATGTTACTTTCTTCAACGACATAAGCATTCAATCCAAGAAG
CCTCGTAGCCATGACTATCTTATCAGGCATAGAATAACCGGCATCCGCGATATTATCTTTGTGTATTCTC
GGATTATAATTACCACTAGTTATTAAGTATAAATCCCGCTCACAACGATTATCCAAATCAAGAGAACTCT
TCCTTGTCATTTCGGACATAGACCCTTTGTATTGTGGTATTTTATCAACCCCTAATTCCTTTGCAGCACAG
AGTAAACTGGCAGCTCCACAAGAATTGTCATACATCTGCTTCCTCGCCAAAAAACAACCTTGTTCGTTTA
CTCTATTCTCATTTACCCTCACATCAGTCTGATGAGATATCATATATTCTCTTGGTATAGCCCCCTGCAAT
AAAACCCCATATAACATAACACCTGTATGAATACATCCCCGTTTGCAAGGCTTCCGTTGTTCCGACATCG
ACGAGTAATTGCAGCACAGTATTGCATCCAGCAATGCGAACTACTATGAGAATTAGCTGACCGGGTCCT
TATTCCCCGAACGAACTTTAAATCCAAAACCTCATACTCTTTTCCGTCGTCATCACGTCCGTCACCATAAC
AACCTTTCAGCATCCACCCTTTTATTAAGAGTTAATGTCCATCAGCATCAGCAATTAATACCGCCTGATA
CTGACATATTTTTGTCGTATTTACGTCCATAATCGGTTATTGTGATTCAGGTCACACCAGCTTATTTGCTA
TTGTTACAATAACCACCTTGGTTGAGAGCTGTTTTTTCAACTTTGTTATCAACTCCCCCAACTCCTTGGTC
AGTAAGGCTGCCGACCGAGCACAGTGAATAGATAGTATCCGCAGGTACCACCATGCTTACTTAATACTC
AGAAGGCGAAGTGCTCTCCAATTAACTCTGCCGCACTATGCTTATAAGCTGCTTTTCAATGCCGTCTATC
AACATGTCCAGTTCATTCAATCTGTTGTACTGGTAATCCAACGCAGTAATGATATATGCAGGTATTCTTT
CATCCACTCCAGTAACGTTTCATGCAAAGCATTAATTTACGCGCTACGGAACTTCACCAGTTGACTGAGC
AAACGATGTGGGACCAGTACTGCCTGCAGTTCTTCTATTGTTACAGCGATTTCTTTACCAAACAGCTGAA
CCACCATCAAGATAAAGATAGCCTGAACATTCATCGCACCATTTTGTTACCAATGGCAAAAGATTTAAC
GAACCGTGCCTGCATCAGGCGGACTTTTTATCCCTGCTTTGTTAGCTCCCTAACCTTGTACAGAGAGCCT
CCATAAGCTCCCATATCAAGCAGACATGGTTCGCAGTTACTGACATACACCAGAAAACTCTGACAGCAG
ACTCACTTATCCTCCACTTCACTCATATCCTTATTGGTGAGATGAACCTGAATGACATGTTTTGCGACATA
AACGCCAACCAGTGTATATTTCACCTGTAGAGATTCTACAATCTGTAGCGTTATGCATCTAATGTTAGGC
TCTATGATGTCAGAAATCTGCGAGACTCTACACCTTCTGCATTTACCTTTATTTATCACCATGCTCCTCAG
TTAACGGAGAACGTGTTCATTGCATTTTGTCACTTACATTTCGCCTTTGGTACCGGGGGGATATAGGTTC
CAACGTTCCGCATGAGAACCTGAACCGGCTTCACGCCACCTTGATACCAGATGCCGCACAGGCAGTCAA
CAAGTGTCACGAACGGTGCAATAGTGATCCACACCCAACGCCTGAAATCAGATCCAGGGGGTAATCTG
CTCTCCTGATTCAGGAGAGCTTATGGTCACTTTTGAGACAGTTATGGAAATTAAAATCCTGCACAAGCA
GGGAATGAGTAGCCGGGCGATTGCCAGAGAACTGGGGCTCTCCCGCAATACCGTTAAACGTTATTTGC
AGGCAAAATCTGAGCCGCCAAAATATACGCCGCGACCTGCTGTTGCTTCACTCCTGGATGAATACCGGG
ATTATATTCGTCAACGCATCGCCGATGCTCATCCTTACAAAATCCCGGCAACGGTAATCGCTCGCGAGA
TCAGAGACCAGGGATATCGTGGCGGAATGACCATTCTCAGGGCATTCATTCGTTCTCTCTCGGTTCCTC
AGGAGCAGGAGCCTGCCGTTCGGTTCGAAACTGAACCCGGACGACAGATGCAGGTTGACTGGGGCAC
TATGCGTAATGGTCGCTCACCGCTTCACGTGTTCGTTGCTGTTCCCGGATACAGCCGAATGTTGTACATC
GAATTCACTGACAATATGCGTTATGACACGCTGGAGACCTGCCATCGTAATGCGTTCCGCTTCTTTGGT
GGTGTGCCGCGCGAAGTGTTGTATGACAATATGAAAACTGTGGTTCTGCAACGTGACGCATATCAGAC
CGGTCAGCACCGGTTCCATCCTTCGCTGTGGCAGTTCGGCAAGGAGATGGGCTTCTCTCCCCGACTGTG
TCGCCCCTTCAGGGCACAGACTAAAGGTAAGGTGGAACGGATGGTGCAGTACACCCGTAACAGTTTTT
ACATCCCACTAATGACTCGCCTGCGCCCGATGGGGAGCACTGTCGATGTTGAAACAGCCAACCGCCAC
GGTCTGCGCTGGCTGCACGATGTCGCTAACCAACGAAAGCATGAAACAATCCAGGCCCGTCCCTGTGA
TCGCTGGCTCGAAGAGCAGCAGTCCATGCTGGCACTGCCTCCGGAGAAAAAAGAGTATGACGTGCATC
CTGGTGAAAATCTGGTGAGCTTCGACAACCCCGTTACTTTATTCGTACCCCTTATAATGGGGTGTTAGCC
AGCCTGAGGTGTACTGGCAATAGCGGACACCACCATTTGTTCTTTTTCTAAGCAGTCATCTGATAATATT
TTATCCTGAAGGCTGCCGGGGAGATATTCCCCAGACGAGAGTGACGACGCTGACGATTGTAGAAAATC
TCAATGTATTCCCGTATTACTGAGATGGCCTCATCCCGGCTCTTAAAACGATAGTGGCTCAGGCTTTCAT
TTTTCAGCGTTCCCCAGAAGCTTTCCATCGGAGCGTTGTCGTAACAGTTACCTTTACGCGACATTGATGT
TTTCAGACCAGACTGCTCCTGTATGACCCGGTAATCGTATGCGCAGTACTGTGAACCTCGATCGGTGTG
GTGGATTAGCCCGGCAGGTGGGCGCTGGCTCCTGAGCGCCATAAACAGGGCTTTACCCGTCAACTCTTT
CGTCATGCGCTCTCCCATGGCGTAACCGACAATTTCGCACGTATAAACATCTTTGACGCCGGCGAGATA
CAGCCATCCTTCCCGGGTGGCAACATACGTTATATCTGCGACCCAGACCTGATTAGGTGCTGTGGGCGT
AAAGTTCTGATTCAGCAGATTCGGCGTAACCGGCAGATTGTGGTCAGAGTTTGTGGTCGCCCTGAACTT
GCGTTTCTGCTTACAGTGCAGCCTCAGCTCCTTACGAAGACGTGCCAGTCTGTCACGACCGACGATAAT
TCCGTTATCTGCCAGCTCCGTCTGGAGCCGCCGGGTTCCATATGTTTCGCGAGTGCGGATATGTGCCAC
CTTAATCTCCAGTTTTAGCCGCTCATCACTTTGTTTTCTGTCTGAGGGGACATGCTGTACCCGGTCGTAA
TAACCGCTCCTGGATACACCAGGTACCCGACACATCGCTTCAACAGGGAATTGTTGTCGCCATTGTTCG
ACTAACGCGTATTTTTCAGCGACTCCTGGGCAAAATACGCTGTTGCTTTTTTTAATATATCTCGCTCAAGT
CGTGCTTCATTTAAAGCCTTACGCAGTTGCATAACTTCAGATTCCAGCTCAGCCACTGTGCGGGAGCCT
GGTGTATTGAGCCCTTTTCTTGCGGCGGTAACCCATTGTCCTAAAGTGCCTTCAGGAACAGATAATCGG
GAAGCGCCTTCACTGATCGAAAGCTGATTTTCAGGAACCGTTTTGACGGCTTCGGCTTTAAACTCCGTA
GAGTAACGTTGGGTTTTTCTGCTCATTATTGGCTCCTTCTGATGCCATTCTATTTCAGGAAGGAGTGTCC
GTTAAACTCAGGCTACCTCACTCCCGTAAAGACGGGAGTCAAGTTCCCGCTCCGACATAACTGCGTGCA
ATGGATTTTGAAGGAACCGTCAGCCCATTCCCCGGAGGCAAACATGGGCTGGCAAAAGTGTAGCGGTA
TTAGGCACAGCTATTTAGGAAAGAGGGACCATCTCCATCTCATTAAATGGACCACGCCAGACAATTCAC
TGTCATGCTGATGCTGCACTGTGGGGACAGAATCACTCATGATGACTTCGATAATCGACGACATTATTA
TTTGGCCGAGCTTTTAGGTCATATCTCAAATCGTTTGCCTAATATTGCTCCATTTTTTAATAAAAAATCAA
TCATTTTTGAATCTTTGCTTTTCATTGCATTGTCAAGCATAGTGTCCCCACTATTTGCTTTTTGAAATTTTT
TGTTTACATCAACCAATCCATTATTGATAAAATATTCTAGCACTTTATAGTTGGCACCATCTTTACTAAGT
AAGTATTCAACATCATACAGATTACAATATCTATTGTTGTTCATTTTCTCCATCTCTGCGACATCTTGTTTT
GTAAAATTAAATTTAGATAGTAAATAAAGTGCTATATCAGCTTTTTTATTGCCTAAAGAATAAGCAAGG
GCCTGATGAGCCATTTTTTTATCAGTCACTTTATTGTTAATCTCTTCATAATTAGATGCTTTAATCGCATCC
TCCAAAGATATGTCTCTGAGTTTTATTTTTTTGAAGTTATCTGTACTTAGCATTCTTGGTATATGATACTC
AAGCTGAAACACAAAGTTTATAATTCTATCCAGACTAACAGGATCAATATTTTTGTTATAACAAAACTCT
CTGAATCCTTGATCTTTACTCTTTCTTATAAAATCAATAAGATGCAACCCAATTCCAAGCCTCATATCTTT
AGTGTTAAATATTGGAACATCAGTTTTCCCCTGATTACCATGCACATACCTGCTAACTTCATCCACAACCT
CTTTAAAGTTATCTAAGAAAAATGATTGGTGCTCATGGTAAAAAACAGGTGGAATAGCATTATCAAAGT
GATCGGTTAATGTCATATATCCATATGGAGAGTCATGATCAATGATATACGCATTGGCGCCAAAATCTA
CAGTATGATGTGTTTTATTTAGTGGGAGTTTTTCTTGATGATTGGAAATTTCAACCCCAAAAAAAACAAA
ATCGTGGTTAGAGATTTCTTTAATATCCTCAGAGTATGTATTCTTATGTGTTTGTATATTTTCAGACTGAA
TTTTATCAAAGGATTTAATATTAAGAATATTATTTGACAGATTAGAGTTACTTTGATGTCTGAAGTTAAA
ATTAGAATTAATAATAATAGAAAGTAAGTGACGTTCGTCTGAAGAAAGAGGATGAACTTTTTGTATTAT
CTCAGAGATATAGTGTCTGGACACCTTATCGAGATGCTCAAGTGACCGAAATGATGACCGTGCTCCAGA
TGGCGCTTCAATAGAAAGATTTAGTGATTTGAAACTGGTCCCTTGAGAAAACATTTTGCTATAACTTTGC
GCTGCTATAGACTTCCTTAAAAAGTTTTTCACAGTATTAGCACAGTGATCTAAATGTTTTTCACTTATGCC
AGAAGAAGATGAGTTTGTTACGCGTAATATATTTTTTTGTAATGCTTTTGTTGCTTTATTCGGATTCGTTT
TTCCATCAACAAGATCAATATTATTCTGAACATTAATATGATTTACTGCTTCAGGTATTTTCATCCCCCCC
CCCAATTATGTGCGTTTTTATCTGTGGTTATTTAGATAACATTATCTAACTGGACACTTTCTTGTATGTAT
TATAGCTAAATGAACAGATTTTAATATGTTGTATGGTTTTCACTCTGAAATCCCCTGAATTTAATCACGTT
TCCTTTACTGAAGGCAATTTTAACCGTAGTATCAATTATTAACCCTTGGGCTTGATATCCCTCTCCTCTTC
TGGGCACGGGGGATGTCAGATTACTTATTACAGAAACCAAAAGAATTGTAAGCGTCATCTCAGTGTCG
TCTATTTCAATGTCTGATGATTGGCGATGATAGAGTCCTCTTATAAGTAAGCGCCCCATCAGCGACGTCT
TGTGAAAATTGTCCTGTCTGGCAACAATCGCGCCCATCTATATTGATGGACACGAACGATGAATTCCCA
GACAACAAAAGATATTCCCTGCTTCCGTTCTTATTTGCCTGATGCCCTGCGTTTAAGATTTGAAGATAAA
CTGACCATCCGGGCCATCGCTCAGCGTCTGGGTCTCAGTCATTCCACAATACATACGCTTTTTCAGCGAT
TTCTTGCATCCGGTATCGCATGGCCATTGCCCGATTCAGTTTCATTCGCTCAACTTGACGCCATCCTTTAT
GCCAACAGAAAGAAGGAATTAACAGAGCCTCAAATCAGAGAAGGCTCATGGCGAAAAGAACGGCGAA
CCAGCTATAGCCGTGAATTTAAGGTCCGTCTGGCTAAGCAGGCATTACAGCCTGGTGCTGTTGTTGCCC
GGATCGCCAGAGAACACGATATCAATGATAACCTGCTGTTTAAATGGAAAAGCCAGTACGAGGACGGC
TTACTGAGCGATGATGACATACAGGAATGCATGCCTGTCCCGGTGGCACTGACTGATACGCCGGAGCC
GACCAGACCAGTTACAAATCCCTTCTGGCGTAAGAACCACGGCAGCCTGGCGGCTGCAAATCGGGGTG
TGGCAGAATATGAGCTTTCAGAGTAACCATCCAGAATGAGTTCCCATGTCACACAACCTGGAGCACCAG
AAAGTCCACACCAGAATGGTGAAAGAGGTTCTGAAAGCCGTCGCCAGAGCCAATAATCACCCTTATCA
GTCCGTATTTACAGACTTTATCGCCGGTCATCCCTCCTGCACGGTCTGTTTCTGGGAAACCTTCCACAAA
ATGTACCCGGATTCACCGTACGAATACGTCACGTTCTGCCATACCTGCCGTCGTTTTGATTTATACGAAA
CAGAAGCGGAGATGAAGGCGGATGACCCGAAGTGGTGGTGATTTTCAGCCCCGCCCGCTGAAACGTC
TGTTCACGGCCAACCAGTGCTGGACATCCTTCCTGGATGCGGGCGGTCTGCGCGATATCGAGGTTGAG
GCTGTCACAAAAATGCTGGCCTGCGGCACGCGGATACTGGGAGTGAAGGAATACAACTGCGATAAAC
CTGAGTGCCCCCACGTCAGATACGTCACTAACTCATGCGGCAGCCGTGCCTGCCCGTCCTGCGGAAAAA
AGGCCACAGACCTGTGGATAGCGACACAGCTGAATCGTCTTCCTGACTGCGACTGGGTACATCTGGTCT
TCACCCTGCCGGACACGCTGTGGCCGGTGTTCGAAAGCAACCGGTGGCTGCTGAATGACGTGTGCCGT
CTGGCGGTGGAGAATCTGCTGTATGCCGCCCGGAAACGGGGGCAGGAACCCGGTATCTTCTGCGCCAT
CCACACGTATGGCCGTCGTCTCAACTGGCATCCGCATGTACATGTGTCTGTAACCTGTGGATGTCTGAA
TAAGCATGGTCAGTGGAAAAAGCTGAGCTTCCTGAAAGACGCGATGCGTTCGCGGTGGATGTGGAAT
ATGCGGCAACGGCTTCTGAAAGCGTGGTCAGAGGGGCTGGCGATGCCGGAGTCGTTGTCACATATCAC
GACGGAATCACAGTGGAGAAGCCTGGTGCTGAAAGCCGGAGGAAAATACTGGCATGTGTACATGTCG
AAAAAAACGGCCGGCGGGAGGAATACGGCGCGCTACCTGGGTCGTTATCTGAAGAAGCCGCCGATAG
CGGCCTCCCGGCTGGCACATTACAACGGAGGGGCGAGCCTGAGCTTCCGTTACCTGGACCACAAAACG
GGAGAAACGGCGACGGAAACGCTGACACAGCGTGAGCTGGTCGCGAGGCTGAAACAGCACATCCCG
GAGAAGTTTTTTAAGATGGTGAGGTACTTCGGGTTCCTTGCCAACCGGGTGTGTGGAGAGAAGCTGCC
GCAGGTGTACCGTGCTCTGGGGATGGATAAACCGGAACCAGTGGCGAAAGTGTGCTATGCACAAATG
GTGAAACAGTTCCTGAGTCGTGACCCGTTCGAATGCGTGCTGTGTGGCGGCCGGATGGTATACCGCCG
GGCCATTGCGGGGCTGAATGTGGAGGGGCTGAAGAAAAACGCGCGGGATATCAGTCTGCTGAGGTAT
ATGCCGGCCTGACACAGGCTAAGTGTGCCCGGGAATCGGTGCAAGTATAAAAAACAGCCTGTTGCGGT
ATAAGAATCGACCATGCAGAGAGAGAAAACACCGGAGTGGTGGGGAAAAACAGAAAGGCTCACGTG
GCATAAGGCGCGGACAGGGGTATCGCTTAGTTTTTCAATTTCCTATACGTCAAGCTGTGCACGTCCATC
CGATGCCATTTTCTCATTTGACTTTCGGGGGATTACTGCCTTTATACCGTTATTTTTCAGCTCGTTGCGCA
ATGCGCGCCCTGAATACGCTTTATCAGCCAGTACCGCATGTCCACGGCGTTTCATGCTGCCGTTCTGAC
GCTGAACGCCAATCCCGTCCAGAAGTCGTTGCGCGAACTGGCTTTCATGAGCTTGTCCGGGGCTTAGCA
CGATATTTAACGGGAGGCCACTTCCGTCTGTTGCCAGATGGATTTTGGTGCCAAAACCGCCGCGAGAG
CGACCCGGCCCATTATCTCCGTCGATATCGGGATGTTTTTTTGAGCACCGGCGGCACATTTCAGCGCCC
GGATATTACTGCCATCCAGCGCGGTGGCAGACCAGTCAATAAAGCCGTTTGCATCAAGGGTAATGACT
CCAACTTATTGATAGTGTTTTATGTTCAGATAATGCCCGATGACTTTGTCATGCAGCTCCACCGATTTTG
AGAACGACAGCGACTTCCGTCCCAGCCGTGCCAGGTGCTGCCTCAGATTCAGGTTATGCCGCTCAATTC
GCTGCGTATATCGCTTGCTGATTACGTGCAGCTTTCCCTTCAGGCGGGATTCATACAGCGGCCAGCCAT
CCGTCATCCATATCACCACGTCAAAGGGTGACAGCAGGCTCATAAGACGCCCCAGCGTCGCCATAGTG
CGTTCACCGAATACGTGCGCAACAACCGTCTTCCGGAGACTGTCATACGCGTAAAACAGCCAGCGCTG
GCGCGATTTAGCCCCGACATAGCCCCACTGTTCGTCCATTTCCGCGCAGACGATGACGTCACTGCCCGG
CTGTATGCGCGAGGTTACCGACTGCGGCCTGAGTTTTTTAAGTGACGTAAAATCGTGTTGAGGCCAACG
CCCATAATGCGTGCAGTTGCCCGGCATCCAACGCCATTCATGGCCATATCAATGATTTTCTGGTGCGTAC
CGGGTTGAGAAGCGGTGTAAGTGAACTGCAGTTGCCATGTTTTACGGCAGTGAGAGCAGAGATAGCG
CTGATGTCCGGCAGTGCTTTTGCCGTTACGCACCACCCCGTCAGTAGCTGAACAGGAGGGACAGCTGA
TAGAAACAGAAGCCACTGGAGCACCTCAAAAACACCATCATACACTAAATCAGTAAGTTGGCAGCATC
ACCTGCATCAAGTAGCGAAAGCAACCTGTTGAAAATAATATTAATCACGCCTGACTTTGACCATCGGTT
AAAGCGGTTATAAACAGTTTTCCATGACCCATATCGTTCGGGTAAATCGCGCCATGGTGCACCGGAACA
TAACACCCAGAACATGCCATTGATGATTTTACGGTGCTCAGCCCATGGGCGTCCGGCCCGTGGTGTGGC
GGGTTCAGGAGGTAACAGGGGCTTGATGATGGTCCATGCCTCATCGGGAAGGTCGTAGCAAGCCATA
GTTCAATATGTTGTGTAAACAGACAGTTACTATAACTCAGATGATTAAGGGACACAGCCTACTATTCCG
CGCAGCTATTTAGCTCTCTTTTTGTTGAGCGCCCATGATTCTTTCATTTATTGTTACTAATTTTTTATGTTA
TCATCATGTTATGAGCATGCCCTGTTCTGCGTCTCTATTTTATCTATCATATTCTGAGCATTAACTACATA
ACAAGGCGCAAGATATATATCCAAAAACAAATAATACTGTTGCAATAATTCCTTTCAGTAAAGGAAGTG
TGATTAAACTCAGGGGATTTCAGAGTGAAAATCGTACAACATATTAAAATATGTTCGTTATAATACATA
CAAGAAAATATTAAGTTAGATAATATTATTCTAAATAACTGCAGATAAAAACGCACATAATGGGGGATG
TTTTATGAAAATACCTGAAGCAGTAAATCATATTAATGTTCAGAACAATATTGATCTTGTTGATGGAAAA
ATAAATCCGAACAAAGATACAAAAGCATTACAAAAAAACATATCATGCGTAACAAACTCATCCTCTTCT
GGCATAAGTGAAAAACATTTAGATCACTGTGCTGATACTGTGAAAAGCTTTTTAAGGAAGTCTATAGCA
GCGCAAAGTTATAGTAAAATGTTCTCTCAAGGGACCAGTTTCAAATCATTAAATCTTTCTATTGAAGCGC
CATCTGGAGCACGGTCATCATTTCGGTCACTTGAGCACCTCGATAAGGTGTCCAGACACTATCTTTCTGA
AATAATACAAAAAACTCATCCTCTTTCTTCGGATGAACGTCACTTGCTCTCTATTATTATTAATTCTGATTT
TAACTTCAGACATCAAAGTAACGCTAACCTGTCAAATAATACTCTTAATATTAAGTCCTTTGATAAAATT
AAGTCTGAGAATATACAAACATATAAAAACACATTCTCTGAGGATATTGAAGAAATAGCTAACCACGAT
TTTGTTTTTTTTGGGGTTGAAATTTCCAATCATCAAGAAACACTCCCACTAAATAAAACACATCATACTGT
AGATTTTGGTGCTAATGCGTATATCATTGATCATGACTCTCCATATGGATATATGACATTAACCGACCAC
TTTGATAATGCTATTCCACCTGTTTTTTACCATGAGCACCAATCATTTTTCTTAGATAACTTTAAAGAGGT
TGTGGATGAAGTTAGCAGGTATGTGCATGGTAATCAGGGAAAAACTGATGTGCCAATATTTAATACTA
AAGATATGAGGCTTGGAATTGGGTTGCATCTTATTGATTTTATAAGAAAGAGTAAAGATCAAAGATTCC
GAGAGTTTTGTTATAATAAAAACATTGATCCTGTTAGTCTGGATAGAATTATAAACTTTGTGTTTCAGCT
TGAGTATCATATACCAAGAATGCTAAGCACAGATAACTTCAAAAAGATAAGACTCAGAGATATATCTTT
GGAGGATGCCATTAAAGCATCCAATTATGAAGAAATTAACAATAAGGTGACTGATAAAAAAATGGCTC
ATCAAGCTCTTGCTTATTCTCTAGGTAATGCAAAATCTGATATGGCACTTTATTTACTATCTAAATTTAAT
TTTACAAAACAAGATGTCGCAGAGATGGAGAAAATGAACAACAATATGTATTGTGAGCTGTATGATGT
TGAGTATTTACTTAGTGAAGATAGTGCCAACTATAAAGTGCTAGAATATTTTATTAATAATGGATTGGTT
GATGTAAACAAAAGATTTCAAAAAGCAAATAGTGGGGACACTATGCTTGACAATGCAATGAAAAGCAA
AGATTCAAAAATGATTGATTTTTTATTAAAAAATGGAGCGGTATCAGGCAAACGATTTGGGAGGTGAC
CTAAAATATATTTAGGTTGTGCCCTGAATCTGACGTTGTCATTTTTGTTATATTGATCGATATAGGGGAT
GGTGCAGACCGTTCCCCTGCAACAACACATTGCATGTGGTGAACGCCCCGGGAATCCTGGAGACTAGG
CTGTGTCCCTTAATTACGTAAGCGTTGATAAAAGAGTCGGATGCAGCCCAGTTTCACCATCGCCAGGTA
ATTTCTCGCCGTTTTGTCGTAACGCGTGGCGATGCGGCGATATTCTTTCAGCCGCCCAAAGCACCGTTCA
ACGACATTACGATTGCAGTACGCATCACGATCAAGCTGTGCACGTCCATCCGATGCCATTTTCTCATTTG
ACTTTCGGGGGATTACTGCCTTTATACCGTTATTTTTCAGCTCGTTGCGCAATGCGCGCCCTGAATACGC
TTTATCAGCCAGTACCGCATGTCCACGGCGTTTCATGCTGCCGTTCTGACGCTGAACGCCAATCCCGTCC
AGAAGTCGTTGCGCGAACTGGCTTTCATGAGCTTGTCCGGGGCTTAGCACGATATTTAACGGGAGGCC
ACTTCCGTCTGTTGCCAGATGGATTTTGGTGCCAAAACCGCCGCGAGAGCGACCCGGCCCATTATCTCC
GTCGATATCGGGATGTTTTTTTGAGCACCGGCGGCACATTTCAGCGCCCGGATATTACTGCCATCCAGC
GCGGTGGCAGACCAGTCAATAAAGCCGTTTGCATCAAGTAGCGAAAGCAACCTGTTGAAAATAATATT
AATCACGCCTGACTTTGACCATCGGTTAAAGCGGTTATAAACAGTTTTCCATGACCCATATCGTTCGGGT
AAATCGCGCCATGGTGCACCGGAACATAACACCCAGAACATGCCATTGATGATTTTACGGTGCTCAGCC
CATGGGCGTCCGGCCCGTGGTGTGGCGGGTTCAGGAGGTAACAGAGGCTTGATGATGGTCCATGCCT
CATCGGGAAGGTCGTAGCGAGCCATAGTTCACGTATAGGAAATTGAAAAACTAAGCGATACCCCTGTC
CGCGCCTTATGCCACGTGAGCCTTTCTGTTTTTCCCCACCACTCCGGTGTTTTCTCTCTCTGTATGGTCGG
TTTTTATACAGTAACAGGCTGTTTTTTATACTTGCACCGATTCCCGGGCACACTTAGCCTGTGTCAGGCC
GGCATATACCTCAGCAGACTGATATCCCGCGCGTTTTTCTTCAGCCCCTCCACATTCAGCCCCGCAATGG
CCCGGCGGTATACCATCCGGCCGCCACACAGCACGCATTCGAACGGGTCACGACTCAGGAACTGTTTC
ACCATTTGTGCATAGCACACTTTCGCCACTGGTTCCGGTTTATCCATCCCCAGAGCACGGTACACCTGCG
GCAGCTTCTCTCCACACACCCGGTTGGCAAGGAACCCGAAGTACCTCACCATCTTAAAAAACTTCTCCG
GGATGTGCTGTTTCAGCCTCGCGACCAGCTCACGCTGTGTCAGCGTTTCCGTCGCCGTTTCTCCCGTTTT
GTGGTCCAGGTAACGGAAGCTCAGGCTCGCCCCTCCGTTGTAATGTGCCAGCCGGGAGGCCGCTATCG
GCGGCTTCTTCAGATAACGACCCAGGTAGCGCGCCGTATTCCTCCCGCCGGCCGTTTTTTTCGACATGTA
CACATGCCAGTATTTTCCTCCGGCTTTCAGCACCAGGCTTCTCCACTGTGATTCCGTCGTGATATGTGAC
AACGACTCCGGCATCGCCAGCCCCTCTGACCACGCTTTCAGAAGCCGTTGCCGCATATTCCACATCCACC
GCGAACGCATCGCGTCTTTCAGGAAGCTCAGCTTTTTCCACTGACCATGCTTATTCAGACATCCACAGGT
TACAGACACATGTACATGCGGATGCCAGTTGAGACGACGGCCATACGTGTGGATGGCGCAGAAGATA
CCGGGTTCCTGCCCCCGTTTCCGGGCGGCATACAGCAGATTCTCCACCGCCAGACGGCACACGTCATTC
AGCAGCCACCGGTTGCTTTCGAACACCGGCCACAGCGTGTCCGGCAGGGTGAAGACCAGATGTACCCA
GTCGCAGTCAGGAAGACGATTCAGCTGTGTCGCTATCCACAGGTCTGTGGCCTTTTTTCCGCAGGACGG
GCAGGCACGGCTGCCGCATGAGTTAGTGACGTATCTGACGTGGGGGCACTCAGGTTTATCGCAGTTGT
ATTCCTTCACTCCCAGTATCCGCGTGCCGCAGGCCAGCATTTTTGTGACAGCCTCAACCTCGATATCGCG
CAGACCGCCCGCATCCAGGAAGGATGTCCAGCACTGGTTGGCCGTGAACAGACGTTTCAGCGGGCGG
GGAGTAAAAGCGGACAACATGAGGACGGATTAACCCCCGGTATCCTGTAGCCGGGTAAGGGCTGCTG
TGTCGCCACGGCGCTCCAGGATGGAGGCCGCGACCATCGCAGGTGGTGGGTCCATGGGTTTGAGCATC
TTCATCCAGTCCCATTCACGTTGTGAGAGCTTCTCAGCACCAACAGAGAGCAGAACGGTATCTATACTTT
TGCGGGTCAACATAAGAGGATGAGTCCTTAATAAAACAGGTCCTCAGAGCATACATATTCTGCGGAGC
GTGCGGCAACAACAGGACGAACAGGGGCGGTTTTCAATTTGCAGCCGTCAGGCTGCCGTGGTTCAATA
TGTTGTGTAAACAGACAGTTACTATAACTCAGATGATTAAGGGACACAGCCTGGAACCCAGGATGTTG
CAGGGAAGAGAACTTCATTCAAGCTTCATCATGAGAAGCCGATCAGCCAAAATGGTGGTGTCTATGAT
ATGGATAACATCAGCGTGGTAACACCTAAAAGGCATATTGATATTCACCGAGGTGAACCGCCCCGGGT
TTCCTGGAGAGTGTTTTATCTGTGAACTCAGGCTGCCAGATCATCGTTTCCGATGGAAGCATAATAAGC
TTTTTCTGCTTCTGCCGGAGGAGTATGGCCCAGCCTTTCCAGCAATCGTCGATTGTTATACCAGTCCACC
CACGTGAGTGTGGCCAGTTCCACTTCTGCACGGTTTTTCCAGCTCTTACGGTGTATTACCTCCGCTTTGT
AAAGACCATTGATGCTCTCCGCCATCGCGTTGTCATACGAGTCGCCTGTACTTCCTGTTGATGCCAGTAA
TCCGGCTTCCTTAAGCCGCTGTGTGTAGGCCAGCGATACATACTGAGAACCTTTATCACTGTGATGGAC
CGTGCCGGACGGTCGACGGGCCCATAACGCCTGCTCCAGTGCATCCAGCACGAATGTCGTTTCCATGG
ACGATGAGACCCGCCACCCCACGATGTATCCGGCAAACACATCAATGATGAACGCCACATAGACGAAG
CCCTGCCATGTGCTGACGTAAGTAAAATCAGCCACCCACAGCTGGTCAGGTCGTTCTGCCACGAACTGA
CGGTTTACGCGGTCGCCTGCGGCAACGGCTTTCCGGCTGATGGTCGTACGGACCTTTTTACCCCGGAGA
ACACCGGCAAGTCCCATAACCGCCATGAGACGTGCCACAGTGCATCTGGCCACTCTGATACCTTCCCGT
AACAACTGACGCCAGACTTTACGCACACCGTATACCTTGTGATTTTCATCGTATACGCGCTGTATCTCTTT
CTTCAGCCAGTCATCGCGCTGCGCACGGGCACTGCGTTTATCCGGATGATGTCGCTGTTGCTGACAGTG
GTAATACGTTGACGGGGCAATATGCAGTTCGCTGCATAGCGGTCCGACCCCGTACTGCTCACGCAGCTT
ATCCAGCAGTGGCATCATTTTTTCCAGAGGCGGTCAAACTCCGCCTTCGCAAAATAAGCGGAAGCCTGG
CGAAGGATATCGTTACTGCGGCGCAGTTCACGATTTTCACGCTCCAGCTCTTTCAGACGCTGACGTTCA
GCGGTGGTGAGCCCTCCATCACCGCCCCCGGTATCCCGCTCATGCTGGCGAACCCGGACACGCAGAGT
CTCCGGCGTACAGCCAATCTTTGGAGCAATGGAACAAATTGTCGCCCATTGTGAGTCATATTCGCCCTG
ACTTTCCAGAACCATACGGACTGCCCGTTGACGGACTTCGGGGGAAAAACGAGTATTTTTAGTCATCCT
GTTTACCTCTTTCTCAGGAAGTTTAGTCTCCAGGATTCCCGGGGCGGTTCAGGCCAGTACCTCGTCAGTC
AACTGACGGTAAATCTGCTGTTCAGTCTCACGCATCACCTGTGCACCGGCTTCCCTCTCCGCATCCGCAT
CACCGCTCAGACCTGATGCTTTCAGCCGGTCAGCCACCCTCTGAGAGTACTCATTCTCCAGCATCTCATA
CTTCTGCTCTTCTGCCTGCGCCCAGCGGTCAGCTTCCGTACGCTTCAGTACAGCATGCCATGGTCCCCAG
AGGGAGAACCAGTCCGTAAATTCATTCTCTTCACGGCTTCTGACCATGGCTTCGGCAGTGCGGAGGTCA
TTTGCTGTCACTCCCGACACGCCATAGAAACGCATTTCCTTCACGGCAGTGGAGAGCTGAAGTTTCTCT
GCGAGCATGGTCTGGAAGGCCAGGTAGACTTCTATCTCATCCACAAAATGGAGAGTTCTGACTTTATCC
CGGGCAATGTCCTCCAGAATTTCGAGGCGGAACATTTCCCTGCCCAGGGAGAGCAGAGCGCCGGTATC
ATTATCGAAAAGGCCTTCTGATGCCTGATGGACCAGGAGGGTTTTCCGGAGATTGTTCCATGTGAGCGC
GACACGGTCCTCACAGCTCTCAGTGGCATCAGCAGCAACAGCGAAAGACTGCTGTCGAAGCTCCGCAG
AGGCACTGAGTTTTTCCAGCCATGCAGCGACCTGTTCACGGAATCCGGAGGTATTGCGTGCAGAGACG
GTATCGGAAAGGCGGTCAAGGAACGCGGAAAAGGTGTTGGCATGCTCTTCATGTTCAAAAGCATGCCA
TATCTGTGATACATCAGATTGTTTGTTTTCCGGGAACCATGCTGTCACGGCATCAGCCAGGGGGCGATG
GAGTGTATTCTGTTGTCCGTCACTCATGGAGAAGTAAATCTGCGGGCCGTGGTAGTCCGGCGAAGAGG
TTAATCTTTGCAGGGATTGCAGAACGCGAGTCGACAATGGATTCCCTGAAATGTTAACATTCGTACAAA
TGGGCAATGTGATTATATTTTCGGGTAATGAAGTTAGTAGGTTATTGGAAACATCCAGTGCCAATAGTT
CTTGCGGTAATCTGGATAATCTGCGTAGTTGATTTTCACCTACCTTCAAATATTTTAAGCGCTGAGGAAA
TTCTGGTAGTTCCTGTAGTCTGTTGCCGGAAACAAAGAGTTCTTGTAGTCTATGAGGCAATTCAGGCAA
GACAGTAAGGCGATTACCTTCAATACGTAATATTGTTAGCGTTGATGGAAGCTCTGGTAACGTCTCCAG
ACTATTAAAACGTGCCGAGAGTGATTGTAAATATGGTGGTAATGATGGCAGACATGACAAATTTCGATT
GAATGAAATATTAAGCTTAATCAGTTGAGATGGAAGTGTCGGGAGTATATTCAAGTTGTTACCATTTAC
ATGAAGTTCTGTCAAAAGCGGAGAATTTTCAGGTAGGGAGATTAACTCATTGTTGGAAATATTAAGTTC
ACGTATATGCAAAGGGAGTGGTGGTAAAGAACGTAGTTTCAGTAAGCTTAAATTGAGAACAGGCTCCC
GGGTATCCAGACATATTTTCAATAATCGAACCGCCTGGATGCGATTTTCTGTTGTTGTTCCTTCCCTAGC
CCAGTCATCCCAGATGCGGTGGTAATCTCTGGTACACTGTTCTTCTACCGATAAACGAGATATACATGAT
AAGCCACAAAGGGAACGAAAAAAAGAATGATTGTTGATCGGTTTCATTTATTAACCCCTCACTATTCAT
ATCTTGGTCTATTAATGCAAATAAAACGAGAAAGTCTTTAAAAAAACAATCCTAGAGCTTTATCGGCGT
TTTCATTCCAGCTGGAGGAATATGTTCCTGTCTTTCCAGTAACCGTCGATTGTTATATCAGTCTACCCATG
TTAATGTGACCAGTTCTACTTTCATCCAGTTTGGATTGATCTTTATCCCCTATATTTTCAAACAATTTTTAT
CATTTAACTCATTACTGTCTCGATATTCCCATAGCAAACGATCTGGTTGGTTAGCCCCTCAAAAAAGTCA
GTTGCACGCCGTAAATCCCTGCCGGTATTGAGCGAGGATTTACGGGTTGTTGTTAAGGTCCGTAATCGG
GTCACAGTTTTTCCGGAGTCAATCCGGTCTGCGGTTTATGCTTATGCGACGTGATTATGAATGGTGCAG
TCGTGAGCCGTTTTCAGACAATCGCAGGGCCAGTACCTCGTCAGTCAACTGACGGTAAATCTGCTGTTC
AGTCTCACGCATCACCTGTGCACCGGCTTCCCTCTCCGCATCCGCATCACCGCTCAGACCTGATGCTTTC
AGCCGGTCAGCCACCCTCTGAGAGTACTCATTCTCCAGCATCTCATACTTCTGCTCTTCTGCCTGCGCCC
AGCGGTCAGCTTCCGTACGCTTCAGTACAGCATGCCATGGTCCCCAGAGGGAGAACCAGTCCGTAAAT
TCATTCTCTTCACGGCTTCTGACCATGGCTTCGGCAGTGCGGAGGTCATTTGCTGTCACTCCCGACACGC
CATAGAAACGCATTTCCTTCACGGCAGTGGAGAGCTGAAGTTTCTCTGCGAGCATGGTCTGGAAGGCC
AGGTAGACTTCTATCTCATCCACAAAATGGAGAGTTCTGACTTTATCCCGGGCAATGTCCTCCAGAATTT
CGAGGCGGAACATTTCCCTGCCCAGGGAGAGCAGAGCGCCGGTATCATTATCGAAAAGGCCTTCTGAT
GCCTGATGGACCAGGAGGGTTTTCCGGAGATTGTTCCATGTGAGCGCGACACGGTCCTCACAGCTCTC
AGTGGCATCAGCAGCAACAGCGAAAGACTGCTGTCGAAGCTCCGCAGAGGCACTGAGTTTTTCCAGCC
ATGCAGCGACCTGTTCACGGAATCCGGAGGTATTGCGTGCAGAGACGGTATCGGAAAGGCGGTCAAG
GAACGCGGAAAAGGTGTTGGCATGCTCTTCATGTTCAAAAGCATGCCATATCTGTGATACATCAGATTG
TTTGTTTTCCGGGAACCATGCTGTCACGGCATCAGCCAGGGGGCGATGGAGTGTATTCTGTTGTCCGTC
ACTCATGGAGAAGTAAATCTGCGGGCCGTGGTAGTCCGGCGAAGAGGTTAATCTTTGCAGGGATTGCA
GAACGCGAGTCGACAATGGATTCCCTGAAATATTAATCCTGTAACTTGAATCTAATTGTGAAAAACTCT
CCGGAAGAGTGGTTATCTGATTAAAATGAAAATAATATTCCTTTTGTCTGATATTGTTTCTATCAGAAAA
TTGAGGAAAAGTAACCAGATTATTGCGCTGAGCACAGAGAATTTCCAGTTTATCCGGTAAGTCGGGTA
ATGTTGTCAGCCTGTTATAATAAACATGTAAAAATTTCAATTTTTCGGGAAGAGCCGGTAAGTTATATA
GCCTGTTATTTTCAACAAATAATGATGTTAAATGGTTTGGCAAAACAGGAAGGTTTTCCAGTTGATTAG
AACGAACACTTAAAGTTGTTAATGACTCAGGAAGTTCTGGTATCACAGAAAGCCTGTTATTATCAGCAT
TAAGTTCTTTGAGAAATGCAGGCAGCTTTGGCAATGATGTTAATTTATTATTTTCTACAATCAAAGCAGA
AATATGCTTTGGTAATTCGGGAAGAGACGTCAAATTTAGGTCTGATAAATTTAATACTGCTTCTTGATTC
TGAAAGCACTGGGAGAGTCTGTTAAAAGCAATGCCTCGCTCTTCCCCGGGAGAAGAGTTCTTTTCCCAT
TCAGTCAGGATTCTCAGATAATATTCATTACTGGTTGAGTTTGAGTTAATAGAGGGGGAGCAAGAAACT
GATGAGTGTGTATTATTTACAGAGAACATAATGETAGGTCAACCTTATATTTGTGAGAATAAATATTTAA
AATTGTATATTTATACAAATTTCACTTACCATTTCCGATATTAAGTTTTAATAGTTTCCATTTTACACATAA
CAGAAGTTAGTTGTAATTTATTTATTGAGGTTTTCTATGGAATTCTTTTTTTATTGTGACTGCAGTCAATA
ATAACACTGAATTCAACATTCCAGTGAGCAAGGAAATGTAATCCGAGTAAGTATATTTGTTTTACATCAT
ATTTTAGCCAAGCTCATTTATTCCTATTATAGAATGATAAATTAAAATTACTGTAAAAAAACGATACACA
TTTAATACGAGGGAGGCCGGCAGAAAAACGGCTTGCGGTAAGGCAGGAGAAATGTGTGCCGCGAATG
AAGTCCTTGTATGACTGGATAAAGCTCCAGAGGAAAACTTTGTCAAACCATGCTGAAATGGTAAAGGC
GTTCAACTATATCCTGAATCACTGGAATGCACTGAATGAGTTTTGCCGGGTAGTACTTCCAATCATTAGA
ATATGTGCTTTTCAATGAAGGAACCGATGACTTTTTCGTGGATTTCCACGGAACGCGAGAAGCAGATTG
TTTTACGTGCCAGCCGCTTGATGCGGGGGGGGAGGGTCAGGTTGTTACGTTCATACGTTGCGTGAAGA
TTTTGCCGGTCAGGTGTTTCTCCTCCGGCACTTCTCTGGCGTAGCTGCCCCAGTCATCGCTGGTCACCAT
GCCACCATGCCGATACAGAACGGAGTCAACAGGGCTAACAGTTCCCGACAGGTCTCATCATTCCGTGG
CCCGCAAGTGTAAGCCAACACACCGCCAGTTTTAGTGTTGTACGCATACCAGGGCCAGTGTTGTTGCGC
CTTGCTGCCAACGAAGCGCCACTGCTCATCAAGTTCACAGATAAGGGCAACATCAGCGTGAGCGACTG
GCGCAGATGTTATCCGCCGGGGCGAGAGCTTTTTAATGTCCGTATGACGGTATTGATGCCGATTTTCAG
TGTTCTGGCGGTATAACAGCCCCCTGCGCCGTTATGTGCCATTTCAACGATCTGCTCCTTAACGCCGGGT
TTTCTGGCTTCATAAGAGTAAGTGAGCTGGAAAACACGTTTACATGAGCGACAACGGAAACGCTCATG
CCGGGAACAACTACGGCCATGTCGATAAACTTCATCGGAATGACAGCGCGGACAATGAACAGTAACGG
TAGTCATGGAAAACCTCAAATACCAGTAGATTACCAGAAACTTCTATCGATTGAAGACATTACCTTTTGC
CGCAATAGGTGGGGTAACATTGGTGAAAACGCATTACGCCGGGTGGCAGTTGGAAAAAAATTATCTTT
TTTCGGCTCTGATCGTGGTGGAGAAAATGCGGCAATAATCTAAATAGCTGCGCTTAATACCGCTACACT
TTTGCCAGCCCATGTTTGCCTCCGGGGAATGGGCTGACGGTTTCCATAAAATGGCGAACTTTTTTCAAC
AGTTGCCACATTGAGCTGCACTGATGATTACGCGTTATTGTGTCGTGAAGTGCCTGCCATAGCCGTTCA
ACATGATTCACCCATGGCGAGTAAACCGGCTGATAAATGACCCTGAACTTCGGGTTCTCCTTCAGCCAG
CTCTGTGTTTCCCGGCTTTTGTGGATAATGTAGTTGTCCACGATCAGCGTGATGGTTTTCGCCCGACGGT
ATGTCGCTTTAAGCCGCTTCAGCAGGCTGATGAACAGCGCCGAACTTTTGCTGTTGCCGCCCACACAGC
TGACTTTACCTGTCCCGCTGTGCAGCGCTCCGGCCAGATAATATTTTTCATTCTGTCCCGGCGTGACCAC
CCGTTTTTGCTGTCCGCGCAGTTGCCAGTCCGCACCGATTTTGGGATTAAGATGGATATCCACTTCATCT
TCATAAAAGACCTGATGTTCTGCGCTGCATTCGTCCAGTGCTTTATGGATTGCTGCCATCTTTTCATCTTT
ATGCGGGTCACGGATACGCAGAGTTGGCGCAGCCCTTCGCCACACAATCCCCGCAGACGGCAACCAGC
GGCGAACGGTTCCGGCATTTAACTGGCAACCGGTTATCTCATTGATTTTTATTGCCAGCAGTTCTGTACT
CCAGCGTGAACGCTGGTAGCCAAAGTCGCCGGGAGAATGTTTTACCAGCTCACGTAACAGTGTGCAGA
TATGCTCAAACGGCCAGCGACGGGCACGCCCGGCAGGTAATGATTTCAGTCCCTCAACACCCGACTGC
GTGAACCAGTTAATCCAGCGTCCAACAGAGGAACGGGCGCAGCAGAGCGTTCTGGCAACGTCGCTGA
CACGGTCGCCCCGGTGCAGCATCAGCATGGCAGTCAGTCTGCGGGCATAATTTTTATCGTGTGTTTTAT
GGATGGCTTTCTGCATCAGGCGTCGTTCGTCACGGGAAATTGGTGCTATGATCGGCATTGCTCAGTCCG
GTTGGTGATTTGTTTTGATTTGGCGATTGATCAGATCGCACAATCCGGGCTAAGTTCCCTCAAAGGGAT
CTACTATTCCGCGCAGCTATTTAGGCTGTGTCCCTTAATTACGTAAGCGTTGATAAAAGAGTCGGATGC
AGCCCAGTTTCACCATCGCCAGGTAATTTCTCGCCGTTTTGTCGTAACGCGTGGCGATGCGGCGATATT
CTTTCAGCCGCCCAAAGCACCGTTCAACGACATTACGATTGCAGTACGCATCACGATCAAGCTGTGCAC
GTCCATCCGATGCCATTTTCTCATTTGACTTTCGGGGGATTACTGCCTTTATACCGTTATTTTTCAGCTCG
TTGCGCAATGCGCGCCCTGAATACGCTTTATCAGCCAGTACCGCATGTCCACGGCGTTTCATGCTGCCG
TTCTGACGCTGAACGCCAATCCCGTCCAGAAGTCGTTGCGCGAACTGGCTTTCATGAGCTTGTCCGGGG
CTTAGCACGATATTTAACGGGAGGCCACTTCCGTCTGTTGCCAGATGGATTTTGGTGCCAAAACCGCCG
CGAGAGCGACCCGGCCCATTATCTCCGTCGATATCGGGATGTTTTTTTGAGCACCGGCGGCACATTTCA
GCGCCCGGATATTACTGCCATCCAGCGCGGTGGCAGACCAGTCAATAAAGCCGTTTGCATCAAGTAGC
GAAAGCAACCTGTTGAAAATAATATTAATCACGCCTGACTTTGACCATCGGTTAAAGCGGTTATAAACA
GTTTTCCATGACCCATATCGTTCGGGTAAATCGCGCCATGGTGCACCGGAACATAACACCCAGAACATG
CCATTGATGATTTTACGGTGCTCAGCCCATGGGCGTCCGGCCCGTGGTGTGGCGGGTTCAGGAGGTAA
CAGGGGCTTGATGATTCGAGCATTTTCTGCCCGCAGTGCCGATAATTCATCGCTGTCAGATTTGTGAAC
CATGCCCTGCTGTAAATCTCCGCATAGATTATGTCTGTTTGATCTCAGTCATCCGGTGTCTGGCGAGAAG
ACGCACCACGGTGGAAGGGTGAACCCGAAACAGACGGGCGGCATCAGCACCTGTTTTCTGCCCCGATG
TGACGAGCGAGACAATCTCTTTTTGTTGCTGGGGTGTTAACTTTGGTCGCCATCCACCAACCCGTCCTG
GCGTGCCGCTGCAAGTCCACTCTTTGTCCGTTCACGAAGCATGGCGCGTTCAAATTCAGCAAAGGAACC
AACGATTTGCACCAAATACTTTTTACGAGGTTCTTTTCGCCAGTCCTGTAATTCCATACTCAGGTGTCCAT
CTATGTCAGATGGGCGTTTAAGTTACCAATTATCGCTTACTTACGGGGTGAGTATCGTAAAAAAGCGGC
TCTGCAAGTCCTCCTAATTGGTGACATTACCGACCATTTTTCTTCCTTATGTTTTGAGGGTATGTCTTTAA
ATATTTAAAGAAAACTTTAGAAAAATACGATGGTGAACTGTAACCTAACTCAGATGATATGACAGAAAT
GGGAGTATTTGTCCTTCGTAATTTCCTGATAGCTTCTGTAAGCTTTATTTTATGTACAAATGTACTTATAG
AACCGACCTCTTTGTACATTCTTCTTTTTAAAGAAGATTCACTCATTAAAACCGCACTGGCCAACTCGGA
ATTACTCCAGTTACGGGAGTAATTTGCCATCACAATTCTGGCGATTTTTTCAGACACAGAAACAAATGAT
GGTGCATGAAGAATAAGCCCAACATTTTTGTTTTTTTTCAGAATACATGAAAGCAAATAAGAACTCCGA
ACTTTATCGTAAATGGCATCTGTCTCCGTCTTTTTATTATCAGTGCACTGATGAGCACACTGAGATATTA
ACCAGGACATTGCATAGGGTTCATCTCCCCCCCCACTAGAAAAATCAATCAAAACATAATCGGGAGATA
TAAGCTGAGCTTTGGAAATTTTTTTTAATCGTTCAATGTCATGATCAAATAAAACGTTATGTTCACTTATT
TCCTCTGACGTAAATCGCACAACACAAATATCTCTTCTTTGAAGAACAGATATATCCCTGTCAAATAAAA
GAGAAAAAGGAACTAATTTATTAATAAATATACCATTGTATTTTGATACATAAATAACAGTATTTTGGAC
AGATATGTGGAATGGTTCGCCTGATGTATAGATAAAGTAGAAAAACGGCAGAGGGATTTCAATGTATT
TAAATATTTTCGCGTGCGTCCCTGAGTAAGGGACTACAGGAGGTTTATTATTAATAAATATCTGTTTTTT
CATATCAAAGCTTGATTTTTACTTATACATCTTTAGAATCAATCAGATTTTGTAAATCTGCGCCATCATCA
CACCTGATGGTATGAGAAAAGCAGCTCATCTTATCCCGTTATCAAAGCCTAAGACCTATGGTGTCCAGT
CACCTGACAAGCACATCCTCCATAGTTTTCGGTATCATTTTTTCTACCCAAAGTATCAACTTTCAGAAATA
CCGCCCATATTTAACAACATCAACGAACCGTCCAAGCCGAGCTGAAATCCTCTTCCTGAAACAACTCTGT
CAGCCCGCTAATCTGTTTCAGCCTCAGCACTTCATCCTGGTCCATTCCCAGCTCTGTACCAATACGTTCAT
TCGTCCAGCCCAGACGTGAAAGATCCCTGACAATATCAGACATTAAAGTGATCTGGTGTTTCCCCCTGG
CCCGGTTATGTCGAATGGTCGCGGCAATACGTGCGGCCTGTCCCTTTCTCTCCGGATTAATACAGGCTA
CAGGCAACCAGCCCCTCAGGCGTTTTCCTGTATCTGCTTCCCGCCCCAGTAACTGACGATGAAAACCATC
CACCACCAGATAATGATTCTTCTCCTCTGACACCACCACCGGTTGTGTGAAACCATCTTTCTCCAGAGAC
TGTTTCAGTAGTTTTTTCTCACCTAGTGCCATGACATTAGGGTTGTAATCGTTGGCCACCACCTCATCCG
CTTTCACCCACAACACACAGTCCACAGGCTCCTGTGCAAAAGGACTGACCTCATGAAGCACTCTGCGCA
GATGATTCAGAGCCTGTATGCTCTCTTTTTCAGTTCGTACCTGACTGAAATACTGACGAATCATGTTGCC
AAGTATCTCCGGTGTCACAGAATCCCCCATTCCTTCCTCCTTTCCTTCATACGCTGCAGGTAACGTTCATA
GTGCCGGGGTTTGTTCGGACTGAAGGAGAGAGTCCGGCACCAGAAATCATTCTTTATGAGTGTCTTAC
AGATACGTCGCCAGGACGGGATATCCCGGCTCCCCAGGTCATTCTCCTGTTCATCCGGGATGTCATCCG
GGAAGCCCCGCGTCTGATACCAGCGCAGGTAGACAGCAATTTTATTACGGTAATGTTCTGCCGTTCTTT
CCGGCATCACATCCAGCAGGAACATCGCATAGCTACGCCAGGTATGATGAGGAGGCTTTGTGATACGC
TTACGCAGGGCAAAATAGGCACCGCTCTCATTGGCATAAAGCGCCCCGCTGGCAGCACCCGACACCCG
CTCACACATCCTGGCCCAAGTTTCCGGCTCCAGAACATGATAAAGCCACAGCCCCTTACGCTGTTCAGG
ACCAAAAGGCTCACAGACACGCATGTTGCGTAACGGCACACAGGCACGGTGCATCAGGTCATACAGGG
GATTGTAGATAGCTCGGGTTCTGGCGTTATATATCCAGATATCACGGGCTTTCCAGTCATACAACGGAT
ACATGGTGTAATAAAACCCTTCAGGTGATGCCGTAGTCCAGGGTTTATCATCGGCATATCTCTGTTTGC
GACGGGATACCAGTCCCATAAAGCGGTTGAGGGACTCATCTGCCCGGACTCCGGTCAGAACAGCCACC
AGGTATTTATCCCTGGCAAACCAGGAGGAAAACGCCGGGATAAATTCCTCAAAGGTCATGGCATATCG
GTAAAAGGGAAAATACGCCATATCCGTAATAGCATATTCCGGTGGATGGCGAACCCAGGTCACACCAG
GCTCCCAGCATATCCACTCCGGCTGAAACTGAGAGACACCGTTTACCGTAGTCAGGGGAAGGGCCACC
CAGTAAAACCTGTCCGTCACATCCAGGTACATCTTCTGCATCTTCTGAATATGCGTAATGGTGCATTGAT
ATTGTGCTTCCCAGTCAATGAACAATACCGAAAAACGACGCCTCTTTTTGCGGGCCACTTCGGACACAA
GATGGAAAAGCACGGTAGAATCCTTACCACCAGAAAAGGACAAGCAGACAGAAGGAAACGTTCCAAA
TAGCCATTCAATGCGCTGAATGGCTGCAGACAGAACATTTTCACCTGTTTTGATTTTCGGTATGGTTCCT
GACATAATTGAACAAAATATTCAGTACTGGATGCTATTCAGCAATCCTTGATTAGTAAATCACAAAAAA
CATATTAATAATATTCTGGCACAAACTCATTTTTCAAGGGGGAATGCCCATAAAAAAGCAGAATAATTA
ATAATATTATTCTATTTTTTGTGAGCTATAAAGCCCATGGAGAGCAGGAAAAAACTCACTCAAATAGTG
ATAGTTTTAATTITTATCTATGTATACCACTTATTCCAGCCACTCCCGCTATCAACACAACTCCCCAATATT
ACAGTAACCGACGTTCCTTATCTCCGGATTATAAAACGCACTCCTGCTCCTTACTGATATTCAATGTTTAC
TGACAGTGACACCCTCCAGTGTTCCGGCATCTCGCCGTTCAGCCGTAATTCACCACTGCCAGCCACAGTC
TGTGCACCATACACGCCTTCAACCTGGCGCAGGAAGTCATCACTGAAATGACTCACCCCTGAATAACCA
CTGAGTGTCATTGTCTGAAGCCAGCCGGGGCCTTCCCCACTGAACATCTTCTGCAGAACGCCCGGAATA
TCAGAAACCTTCATGGCAGTGCTTTCACCTGGCAAACCATTCGTATTCAGGTCGTCATACATACCAACAT
ACACCGGTTGTCCGCCCTGCACATGGCGCAACACCCCCACAGCCTGAATTCTGAAAACAAATGTGCCTG
CCCGGGCATTATTATCTCCGGTGACAGGTAGCGTCACTTCAGCCATGCCAGGCGCTGTCCATACAAGTG
AAAACCCCTCAGTGCCTTTTCCGTAACTGACCCGGGGTGAAAGCCCCTCACGTCCTGCCGGTGTGGTCA
GGGTGGTTTTTCCCAGTAAAACGGTCATCGCCGGCAGTGCGACCTGAATCCCCCCCCCACCGCCACTCT
GTTTAACCTCCAGACCTCCATTTCCCTGCCCGACTTTCCATACCCACGGGTTACGGGTACTGGTCACCGG
GCCTTCCAGCTTAAGCTCTCCGCTGAAATTTTCTCCTGGCGTGTTCCAGGCCAGTACACAGGAAGGCAG
GTGGCTGGCAGCAAACAGAAACAACGTCACCTTTTTCATTCTCTTCTCTCCTTATATCCCTGCAACCATCA
CTGTACTGTAACCGTCACATTCAGCCCCGCCTGCCATGCTGCAGGTGTACCCTCTGCCGGCAACCGCAG
TTCAAAATCCGACACCTCAGAGGCATATGCCGCAGCCAGATCCATCACCCGACCGTCAGTCAGTATCCC
CTGGTTGATAACCTGTCCGTTATCCACCGCATTCATGCCATTGCCAAAATTCTGATTCATCAGTAACAGG
GAAGAGAGTCGGGCTTTCAGCCCCTGTGGCAGACTTCCCGGCTGTACATCCGTCACACTCTGTCCACTG
ACCAGCGACATGCCAGCCGGTACGGATGCCCCGTCGTCCTGTCTGCCGGTACTGACCGCCATTCCCTGA
TTCAGGGTAAAAAAAAGCTGTCCCGACACGTTCCCCGTCTCCGGGTCACGCACCGGGACAGAGGCGCG
AAAGTGCTGTGCTGAGGCACCATTTCCTTCCGTCACAGTAAAAGGCTGTCCGTTACTGCTGAAAGTAAT
AAATGGCGTGAATCCGGGACCACCGCGCTCAGCCACATCATGCAGATGTCCCTCCAGAAATGGCAGAG
CCCCTTTATCATGCAAATTAAAAACCAGTTGCCCGTTCTCTGTACGGGCATCGGCAGTATCCACAGACCA
GGTCTGGTCGGGAGAACTTATCTGCCAGGCCCATTTGGGTGCTTCATCAGTAACATAGCCATTAAACTG
AATTTCGCCGCCATCAAGAATATTTGCATATGGCACTGCCGAATAAAATATTGTTACCAAAATAACAGC
AGAAAGGAAGAATTTATAATGATGCGTTATTTTCATCGGCATTCATTCCTGTTTATCCGGATAAAACAAA
ACAGCTCAATAAAATAATTTTGTTAAAAAAAGCACAGAACCAATATGCGTCACAAGACACTCCAACAGA
AAAAACACCTGTTCTGACATACCGGTTCTGTATATTCAGGTCGGGTCACCGGAAACTGTGTTCTGAAGT
TTTAATCAGCAGTAAACGGGTAAGTGCTTACTGCTGAACACTGCTGAATTACATGTAGGAGATAACAAT
TGGCAGAGAAGCTTTCCACGGAACTGGGGTAGCAGACGTTATAGTCTGATCAAGAACTAGTTCCATGG
GGATATCCTTGGCAAGGCCGGATGCATAGTAACCTCGGTAAAGCTTGTCTCCATTACTGAAGTCGGTTT
CATCTGTATCGCCCGCCGTGTAAGGAATTTTAGTATCCCACGAGTCAGCAATACTGCTAAAAAGGTGGG
TAGCCCAAGTATAGGCAGCGGCGTCACTCCAGACACCTTTGTCATCTTTGGCAACTCCACCAAAGAAAG
CTTTACCTTTTGAACTGGCGAATAGCATGTTACTCTCTAACTTGTCGTCGCCTTCACCCCTCTTTTCGCTG
GCCTGACAGGCAACTCGCATTACGAACGCCAGAGTACCAATTTTCTTACCATCAGATGATGTGACATTA
ACTTGCGTAGGTAAATTCCCCTTATCCATATAATCCACATCAGCGGCTTTATTATAATCGATTTGTGGGG
CAATACCCTTTTCCCCCATGAAACCAGTCGGGTTGCTACGAATGCCAAGTATCGGTATGTCTTTTTCTAA
AAGAATGAAAACATTCTTCTGGTCAGGTTTAACGGTCACATCAAGCGCTGTTACCGGAGCACCTATTTC
CACTGCCCACGGTACTGTTTTCGCAGCTTTGCTCAGAGTACCACCCATGCTAAAGGTGCCACCGTTGCCA
CCAAGGCCAGAGGGATTCCACGCCATAGCAGAGCCGGATACTGCGGCAGAAGCAGCCACAGTCAGTG
CAATCAGAGTCTTTTTCATAAAATAATCACCTTGACGTTTCATAAATAATAAACTGAATCGGTAATCCTT
ATTTTCCCGGAATTACCGCAAAACCATCCCCTGCGGATTCATTTAAATAACCGCATTTCATTAAACACAG
TGTTTAACTCAGAATAGTTTTACGTTATTCCCGTTTTAAAAGACGGTAAATCCCTTTACCAATATGACCCG
CTCCGGTATCACGGGATGTACTGCATTTATAAAGGAAATGAATCCTCAGTCCTCCACCTGCAGCGCTAT
CTGCAGATTTCCCCGGTAACTGACAGGTACTTCCAGCTCTGCCGGTGCATCCGTTCTTAATTCAATCTGC
TTCTGTGCTTCCGGCAAAGTTATCACCATATCCTCACCCTGCTGTCTGGCGCTGATTGCCACTTTCTTTCC
GTCAACCATCAGGGCCGTCTTCAGGGTGATGCGGGCTTCCCCGTTATCCGGCAGCACCTGTTTCACCGT
GATATACGCCAAGCCACCAGATGCTGCTCTGGATTCTGCATTACGGATGGTCAGGCGGTCGGGAAGAG
GCGTCGTGTCACCACTCCGGTACAGCACCGGATCTGTCGGTATGCGGGTTACCTTCACCACGCTGTTAC
CCACCACCTGGCCATTCACCACCCCCAGATACTGCGCTTTCTCGCTGTATTCAGCGGCAAGTGTTGACTG
AATACTGAGTGCACTCAGGGCCAAGGCGACTACCATCATTGTTTTCTTCATTTATTACCCCTCTGTTCCGT
ATGCCGGAGGGCGATTATTTTTTATCAGATGAATTACCCTCTGTATCAGATGACTTCTGTGCAGACGATA
TTGCTGATTTCTTCTGGTTAATCGTCCATGTCTGCAGTTCACCGTAATCATTCAGAGAGTCCACTTTCACC
ACATTTCCCTTCACCTGTACTTCATCACCCGGCATGAACATCAGCAGTTTCTGCGCCGTCTCGTTATCCGC
GGCAATTTTCTTTCCGTTTCCGTCCAGCAGGCTGCCAATCGCAAAAATGTATGGCGTGGTGTTCACCAG
CATGGTCCTGCCATCCGGGCGGCTCTGCAGACTGATGCCTTCTTCTGCCCCCTTGCGACCTTCAAGCAGT
GCCTTCGGGCGATAGAACAGTTTCAGTTTCGTGCGCAGCGCCACTGCAATACCGCTGCCCTCCAGAGCC
GGCGGAATATCCTGCAGGTTCAGCCAGTACACCGACTCTTTATCCTTCGGCAGATGGTCCGAGGCCATG
ATGATACGCAGTGTCTGCTGACCATTCGGCTTCACCTTGAAGAAGGACGGCGTCACCACAAAAGTCGG
ACGGGTGTCTTTCTCCACGATATTGTCCACCCATGCCTGACCACCAAAGGTACGTTCCTTGTCATTGTTG
GTGACCGTGATGGTCAGCGCATCCTTGTCCCCGCGAAAGATATAGCGGGTCTGGTCTGCCGCCAGGGA
AGCCACCGCACTCTGACAGCTCATCATCAGCGCCAGCGTCATTCCCAGTGCTTTTGTCACCTGGTTAGTG
AAAAACGTCGTTACTGCGTTACGCTTACTCACACGTAATCTCCTGTAGTTTCTCAGTATCCTGCAGTCTG
GCCGCAGGTATCCGGCACTGGCCTAGGGTGATATCACCCGGCGCATCCACCGTGTTAATCATCAGCACA
CCATTGTTAGCTACAAAGCCCAGCGGTGTGTTCTTACTGTCACGTGCCCAGGTTCCCCCTGGCACAAACT
CACCGTCACGCTGCTTCACCTGCAGCAGGTAACGCTTAACTTTCAGGGCATCAAACGGCATCCAGACCA
CCGCCTTGTCCGTCGGCACCACCTTCTGACTGGTGTTCGTCAGTTCGGTGCTCAGCGGCAGTGTGCCCG
CATCAATCGTCACCGTGTTCCAGTCATAGCTGTTCAGCGGTACCACCAGGTTGCCGTCACTGTCAGTCTG
TCCGTCACCGGACGTCACCTTCACACCCGGTGTATCCTTCACGTTCACCACGGCCACGGTGTCCCCGGTC
GTGCGGCTGAACATAATGTCTTTCGCCATCGGGACGACCAGTACCGAACCGCTGACTGATACAGAACC
ACTGGTTCCGCCGGACTGTGAGTGGTTCAGGGCACCATTCAGATAAGCCCGGTCACCACTGTACGACG
CGTTTAGATAACTACTCACACCACCACCGCTGTCACGACCGCCGCCCAGGCTATAGCTGAAACGGTCAT
TCAGCGAGCCGGACACGCCAGTGCTGAAGCCCGTGCCACCGCCTTTACTGGTACTCACCGATGTACTGC
TGCTGTAGCGACGCTCAAACAGCGTGAATGGCACCGAGATAGAGGCTGACGCCGACCTGTTATTCTCT
CCCTGCGAATTCCGTGAGCCGGACCAGCCCAGATTCAGACTGACGCCCTTAATCTGTGTGCTCAGGGTA
CCGGTAATCCCTGTGTCCAGACCATTGTCATCGCTGTACCGATAACGGCCAGTCATCTGTGCCGGGTAA
TAACTCCGCTGCCAGCCGGACACCGACAGACTGAACAGCCCCCCCACCGACTGACTGATACCGGCATTC
CATTCATCCTTCGGCCGGCGACCATGATTTGACTGCGACCACAGCTCTGTCGGGTCAAAGGAGGACATG
TCCTCATATTCCTCACTCTGCCGTGACCAGCTCACCCGCAGTCCGGTGTTCGTCATCTCCAGTTGTTTACT
CCAGGACAACTGCACCTTATTTCCGCTGCGGCTGCCGTCACGGTATTCCGCCATGGCATAAGCCCCGTC
AGCAGACACTGCCCCCAGATAACTCAGCCCCAGAACAGCCCCGGCGCTAATACCCTGCCAGTCCCGGTT
AAACACGCCCCCGGTGCGCAGCGTCAGTCCATCCAGACCGTAACCGTATGACGCCGCAAACACGCCGC
CTTTCAGGTCACTGTCATCGTCAGACAAACCGACTGCCACGCTGAACTCGTGTTGTCCCGGGTTTAACTG
CCCGGCCATCACCGACAACGGGAAGAGCTGAGTCTGCTCACGACCGTCCTCGCCGGTGATTTTCATGGT
CACATCCCCACTGGTGTACAGCGGCACATCCGTGATGGAGAACGGACCTGCCGGCACCATCTCCGAAT
GCAGCAACCGCCCGTTCTGTGTCAGTGTCACCCTCGACGCCCCGTTCGCAATGCCGCTGAACACCGGGG
TATACCCCAGATTGCCCGGCTTCATGCTGTTGTTCCGGCTCAGCGACACTCCGTACGTTCCCGTACTGCC
CAGCAGACTGCTCCCGGTGGATGTTTTCCCGACCGTCAGGTCCGCACTCAGTGCGCGGATGGCCCGGG
TGGCCGTGAACGCGTTTATCGTGGCGGAGTTATCCCCGCTGTCACCACCGCTGGCTGAGGCAGAAGAA
TTCACCACCCAGTGCCCGATATTGGCCTTCAGGTCTGCCGAGCCAAAGGTCGAGGTGTTATTCCGGCCG
ATGTTGGCGTTCGCGTTATAGTTCACGCGAAATGCACCAGTCCCGTAATCCCAGTCCACGTTCTCCGGC
ATCTTCACCAGCCCCTTTTGGGGAATGGACAGCGCCAGACTCTGGGTGGAAACATCAAAATCCACCTTC
ACTGACGGCGCTTTTGTCAGCACATAACACTGCCGTGTGGCGTCATATCCCTCACGAAAGTAATCTGCA
CTGACGTAAATCCCGGCCTTCGCCAGCCATGCCTCTGTCAGGCACAGTTCATCACTGTCCTGTGGTGTCA
CATCCAGTATCTGTTTCCCGGCCTCCTTCCCGTTCAGGGACAGGTCAACCAGATAACGTCCCGGCGCAT
AGCTGCCGTTCAGCGCTGCCCAGACTTCCGGATTAACCCCGCCCCCCCCCCGGATAAAGGACATATCCA
GTTTTTCACCTGCAACACCTGTGGTACTGGCGAAAGCAGCTGATACAGCAAGCGCGGCCCGACGGGAA
AACAAAGACGAAACCAGCTCTGCAACCACCTTCTTTCCTGCACACACAACCCTTTCCTCTGTTACTGATA
CGTGACCACAAAGGTCAGCGCCCCTGTGTATTCCCCTGCCTTCACGTTCGCCGGGAACACCGGCTGTGC
ACGAAAACGGAACTGTCCCTTAACAGCAAACTCCACCGGGTAATTCACCGAATGACTGTCCTGAGTCAG
ACGTCCGCGATAATCCGCCTGTGTATCCACCGCCCGTACATCCACGCGAATACCATCACCAGCACCGCG
AGTGACCACGCCCTTTGCGTCCAGTTGGCCCGGGTTACCAAAATCCAGAGTTGCGACCTGCCCCGCTCG
AAAGGCTGAACAACCTTGAACCGTAGCGCCGGACTCATACAAGCGCACAACAAAGTCACGTGGCAATA
CAGGCACGCCACTGGATTTACGGTAAGTCCCGAATATCAGTCGACCACTGTTGCCGAAACCATCCGCAT
CCACCACCACATGACAGGCAGAACCTACAATATCCACATTGAAAACAACTCGCTGCCCCCCCCCTTTTCC
CGTATTGGAAATTAATAAAGCAGAGACAAACACAATATATTTAATACTCACTAATTTAGAGTTAATAACT
CACACCCCATTTTTATGAAAATTACATAAAAATAATTTAGAAAAAAAATAAACAAATATCCACCCGTTAA
AAACAAAAAAACCAAATAGTGCCAAAACATGAACCATTTTTACTATTTATACTTGCGTTGATTTGGCGTT
TACCTGCGATGCAATTATATAACAATTGATTAATACTTTGATTAATTAACATTTACATACATTAATAATGC
AACTCTATTGAGGTATAATATATAATACGGGAGTTGACATGGGATCCCGTATATCAGCAGTTATTTTTAC
TCGGCAGTTCATTGATTATTATCAATAATAAACGTAAACCACGTGGCCGAAATATTGATGGGATAACAC
CAAGAGGAAAAGGGTGATGACTCTAGGAACATATGTTTTTCGATAAAAGCACCGATAACTTTTTCATGA
AATTTTACAGAGCGAGAGAAACAGATGGTTCTGCGAGCCAAGCGTTTGATTCGGGTTCTCAGGGGCAG
ATTGTTGCGCTCAATGCGCTGAGTAAAAATCTTACCAGTCAGATACTTCTCCTTCGGTAACTTGAGGTAG
CCTGAGTTTAACGGACACTCCTTCCTGAAATAGAATGGCATCAGAAGGAGCTAATAATGAGCAGAAAA
ACCCAACGTTACTCTAAAGAGTTCAAAGCCGAAGCTGTCAGAACGGTTCCTGAAAATCAACTTTCGATC
AGTGAAGGCGCTTCCCGATTATCTCTTCCTGAAGGCACTTTAGGACAATGGGTTACCGCCGCCAGAAAA
GGGCTCGGTACTCCTGGTTCCCGCACGGTGGCTGAACTGGAATCTGAAATTCTGCAACTGCGTAAGGC
GTTAAATGAAGCTCGCCTTGAGCGAGATATATTAAAAAAAGCAACAGCGTATTTTGCACAGGAGTCGC
TGAAAAATACGCGTTAATCGAACAATGGCGACAACAATTTCCCATTGAAGCGATGTGTCAGGTATTTGG
TGTATCCAGGAGCGGTTATTACAACTGGGTACAGCATGAACCCTCAGACAGAAAACAAAGTGATGAGC
GGCTAAAACTGGAGATTAAGGTGGCACATATCCGCACTCGCGAAACATATGGAACCCGGCGGCTCCAG
ACGGAGCTGGCAGAGAATGGCATCATCGTTGGTCGTGACCGACTGGCACGTCTTCGTAAGGAGCTAAG
GCTACGCTGTAAGCAGAAACGCAAGTTCAGAGCGACTACGAACTCGAACCACAATCTGCCAGTTGCGC
CAAATCTGCTGAACCAGACGTTCGCTCCTACAGCACCAAATCAGGTCTGGGTGGCGGACCTGACGTAT
GTTGCCACACAGGAGGGATGGTTGTACCTCGCTGGCATCAAAGATGTTTATACGTGCGAAATTGTCGG
CTACGCCATGGGAGAGCGCATGACAAAAGAGCTGACAGGTAAAGCCCTGTTTATGGCGCTCAGGAGC
CAGCGCCCACCTGCCGGGCTAATCCACCACTCTGATCGAGGTTCACAGTACTGCGCATACGATTACCGG
GTCATACAGGAGCAGTCTGGTCTGAAAACATCAATGTCGCGTAAAGGTAACTGTTACGACAACGCTCC
GATGGAAAGCTTCTGGGGAACGCTGAAAAATGAGAGCCTGAGCCACTATCGTITTAATAACCGGGATG
AAGCCATCTCAGTAATACGGGAATACATTGAGATTTTCTACAATCGTCAGCGTCGTCACTCTCGTCTGG
GGAATATCTCCCCGGCAGCCTTCAGGGAAAAATATCATCAGATGGCTGCTTAAAAAAAGAACAAATGG
TAGTGTCCGCTATTGCCAGTACACCTCAGGCGTGTCAGATCCTGCTCTGATGTACTGACTCTCTCATAGA
TGCGAGCAATTTTCATTTACTGTAGCCAAAATGAGTATAGGTAATTTGGCTATCGTAAAAAAGGTGGTT
AAAAATTACAACCTGCAATGTAAATTGGCTAGTTAATTTTTGGCTATTTAAATTGACTTCTTAACGGCGT
TTACTGTTCCATTGCTCCCCAACCCCCAAGTACAGGCGACTCGTATGACAACGCGATGGCGGAGAGCAT
CAATGGTCTTTACAAAGCGGAGGTAATACACCGTAAGAGCTGGAAAAACCGTGCAGAAGTGGAACTA
GCCACACTCACGTGGGTGGACTGGTATAACAATCGACGATTGCTGGAAAGGCTGGGCCATACTCCTCC
GGCAGAAGCAGAAAAAGCTTATTATGCTTCCATCGGAAACGATGATCTGGCAGCCTGAGTTCACAGAT
AAAACACTCTCCAGGAAACCCGGGGCGGTTCACTGCGTGTCTGGGTTCGCCAGCATGAGCGGGATACC
GGGGGGGGTTCACACGAACATACCACAGACAGTTGATTTTGGTACGCCCAGGCGACGTCCGGCCACAA
CCCGTGGTAAATGTTCTTCGAAATGAAGGCGTAAAGCTTCAGTGATCCAGGTCCGGTATTCCATACGAT
AGTGTCCACTTCAAAATGATGGACATTATTTTTGTGGAACCGGAGGTAACAGACCAGACGGTTTAAATG
AGCCGGTTACCTCAAATTCAGTGTATCACCACGAGATAATATTCAGCTTTTTATATTCTTCATAATTTCTA
GAAAATCACTTCCATTTTTTTTGTCCAAGTGTTCTGCTATTTCTTCAGGTGTAATATTTTCATTTTTTATTA
TTTCCATATAAGTTTTTATTGCCTCAAAATTCCTATTTTGAAAAGCTATAATTAATCCAGATACCCCATTT
GGGCCTTCTGCTGCCAGTAATTTTGATGCCTGATGTGGAGTAAGGTCTGCAGTGTGAAGTATTTTCCCA
TAAGCAATAATTGTTTCTACACATCCATTATGCAAGGCTACAAACAATCCAGATAAACCATTAGAATCTT
TCGCTTCTAACAGCTCTGCTTTTTTATCCAAGTTAAGTTTTGCCTCATTCAAGATATCACCATAAGCTAAG
ATTGCATCAGCCTGTCCATTATTCAATGCTAACAGAAGTCCGGGAACATTGTCATATCTCCTTGAGGCCA
GCAAATTTACAATATCCTCTGAGTTGAGGAGAGGGGGGCTAAGAATGAGCTCACCGTATGCGCGTATA
GCATCTGCATGTCCATTTTGAAGAGCAAACATTAGTCCGGGTATCTGTCTGTGTACTTCATCATAATAAT
AAGCGGTCAGCAAATTTTTCAGCTTATTGTATTCGATATTTATTGCTCTCTTCTTGAGAAGCCTTCCGTAT
GTTTTGATAGTTTCTGCATGCCCATTTTGAAGTGCTTGGGATAAACCCGATATTCGTCCTCCCATTCCTTC
CGCAGTTAGTAGTTCTACCGTTTTATCAAGGTTAAGTCCAGAAGTTTCCAGGAGTTCTCCGTATGCTAAG
ATTGTATCTGAATGCCCATTTTGTAAAGCTAAAAGCAATCCTGACAAACCATCGTCATTTTTGGCTGCCA
AAAGTAATTCTAACGTATTGTCTGAAAGGTCAGATAGTTGTACGACTCTTGTTACTTTTTGGATTAGCTC
AGTCAATCCCATACGCATCGCGTGATAAATAACTTTGGGGTGAAGTAAATTATTAGGCCATCTGATTAT
GGAGGACATGCTTGTTGGAGTATGTCTGTCCACAAATATAGACATACCGTCGGATATAAGACCATAACA
TTTCTGATGTTTTTCATCAAGAAAATTATCTACAGTCAGTTTTTTTATTTTAGCTAAGTTAAATTTGTTAAA
CTCTGCTCGTAAATGAGTTGCTGTATGGTTGGGATCATAAAGCTGAATGACAAAATCCCCCCCATTTTTA
GTTTCTTTAATCTTCAAGCCTAATGCCATATTGTGATTTGATGTACTTATTTCTAGTGCTCTGCTTTTTTCG
GAATGAAAAATCATTTCATTGAAAATATAAAAAATAATATCTCCCCATGAACCGTTTTCAACAAAATAAA
TAATAGCAGGCACATTAATAGCCTTTTCTTCTTCACTCACTGAAACATAATTTTTAATTTCATCCGGAAAA
GCAAAGTGATGATAATCCACGTGACCACTACTTTTGTTAAACTGTGCTATCCAGTAGGAAGCTAAATGT
CGACAGACAATAAGTCTTCCAGAGTGATCGGTAACTTGGCAATTTAGATTTAATAAAAAACTCCTCTTGT
CAGAAAAATATGGCAATTGTTTTGAGTGCAATAGAGACTGGATTGTCTCAACAGAGACGTTGTCTTTAT
TTATCATATTTTGCAAACATATTTTCCTTGATGGAATTAAATTTACTGATGGCATATGTATATATTCCTTTC
ATTGATGGGTTGTGACGCAGACTTAGTGTTGTTGTACTCTTTGCCCTTTGGATGATTGATGGCAAAGAT
TGATGTGGTTTGTCCCCGTTACTCTGAAACTCAGGGGGGTATCCGGAACGGCCATTCCAGTTCAGGGG
GGCTGCTCTATCGCTGTAAGCAGTGCCTGAAGACCTTCCAGCTCGACTACCGCTACAACGGGGCGAAA
CCCATCAAACTTTCCAACTATTATCACATTGAGCCATACTGACAATAAACAATATATCGATATTTTGTGCA
AATACTGCCTTTTGCTAAACGATATTCAATTTTGATTAAATATATTTATTGTCAGATGTTGCACCATATTT
CAATAATAGTITTATCATTTCTGCATTCTCATATTTTATTGCGTTATCCAACATACAATCCCCACTATTAGT
TTTTTTGAACTTTGTGTTTACATCAACCAATCCCTTATTGATAAAATACTCAAGTACTTTACAGCTTGAAT
TATGAGCGCTAAGTAGATACTCAATATCATACAACTCATGCTTTATGGAAATAACATCTTGTCTAGTTAT
CTCAAAATTAGATAATATATATAAGGCGACATCCTCTTTTTGATTAGCAATCGAAAGAAAAAGAGCCTG
GAGAGCAATTTTTTTGTTAGTGACCTGCTTGTTAATTTCTTCGTAATTAGATGCTGTAACAGCCTCCTCTA
AGGATATTTCTCTAATCTTAACTTTTTTGAAGTTTTCTGTACTTACCATCCTAGGTATATGGTACTCTGGC
TGAAAAACAAAGTTTATGATTCTATCCAGATCCACAGGGGCAAGATTTTTTCCATAGCAAAACTCCTTGA
AGCTTTGGTCTTCACTTTTTCTAATAAAGTCAATCAGGTATAATCCGAGCCCTAACTTCATATCTTTAGTA
TTGAATATTGGTACATCTATAATTCCTTTACTTCCATGTACGTATCGACTAACTTCTTTATTAACCTCTGAA
AATTTATCTAAAAATGATTGGTGCTCATGGTAAAAAACAGGTGGAATAGCATTATCAAAGTGATCGGTT
AATGTCATATATCCATATGGAGAGTCATGATCAATGATATACGCATTTGCACCAAAATCAACAGTGTGA
TGTTTTGTATTCAGGGGGTGTTTTTTCTGATGGCTAGAGATTTCAACGCCAAAAAACACAAAGTCATGA
TTAGCTATTTTTTTTATATCATCAGCGTATGTGTTTCGTTTTGTGCATATTCCTTCAGACTGAATTTTATTG
AATGACTTAATGTTTAAAATTTTATCAGACAAATCAGAATTACTTTGATGTCTGAAGATTAAATCAGAAT
TAATAATTAGCGAAAGCAAATTGAGTTCCTCTGCAGAAAGAGGATGAAGTTTTTCCCTTATTTCAGATAT
ATAATGCTTCGATAATCTGTCAAGGTGCTCAATAGCCTTAAATGAGGCTTTAGCGTCTGATGGTGCATC
AATTGCTATATTTAAAGATTTAAAGTTAGAGCCTTGAGAGAACATTCTACTATAACTCTGTGCTGCTATA
GTTTTCCTAAAGAAATCTTTAATAATATTTGCACTATCTTTTAAATTTTTTTCATCTGGCATAGTTTGTTGT
GCAGCGTTTCGCACTGATGTCACTTTTGGAAACAATGTATGTAATCTGTTATCCATAGAATCTATATTGC
ATTGGGTATTTGCTGAGTTCAGTGTTTCTGATATATTCATAAAATGAACCTTATATGAAAACAGTTTTAA
TTAATATCTTTCCCTACAAGATATAGCAGGGTATGAATTACAATTTTTGAGTATATATACAATATACTGC
ATGTAATTAGATAAGGGTGTTATATACTTTTGATGTTATGTGATGCCAGATATTTCAAAATATCTAAAAT
CCGATGCTTAAGAACTCAAGTTAATCTTTTTTGTGAAATTATTTTCATAATTTAATAACTATATCAATTTTA
TTGAACAGCAATGAATTTTACTATAAAAAAACGATACTTTCTCTGATTCCTCTTATGGAGTGCTTCCAGTT
AGTGGAATGTGTATTCTGCAATGAAATCCTCGATGATTTTTTCATGAAGCTCAACTAAGTGTGAAAAAC
TGATGTTTTTTTAATAGAGGTTAGTTTGGCCATATATAATTATTGTACTTTTGCAGTTTTTTTGTTACTTCC
CTTATGAGTTCAAAATCGCCTTAATGGAGCAGTCACTGCAACCCAGAGCCTGTGTGGCGTAGATCGCCC
GTGAAAATGGAATCTGCCGTTCGGTTTTTGCGCGAAGTTTTTCTGAGCTTTTACCGAACTACATACGCTG
CAGTTTCGCAACCAGCGCCTTCAGTCGGTTGATTTCGGAAGCATAAGCCGCCATCCGCTGTGAGAGCAG
GCGGTTGTATTCAGCCATCTGGTGGATAGTGTCCTGTTGCGTCTGCAACAGTGCCCGCAGGCGGGCGT
TCACGTATAGGAAATTGAAAAACTAAGCGATACCCCTGTCCGCGCCTTATGCCACGTGAGCCTTTCTGTT
TTTCCCCACCACTCCGGTGTTTTCTCTCTCTGCATGGTCGATTCTTATACCGCAACAGGCTGTTTTTTATA
CTTGCACCGATTCCCGGGCACACTTAGCCTGTGTCAGGCCGGCATATACCTCAGCAGACTGATATCCCG
CGCGTTTTTCTTCAGCCCCTCCACATTCAGCCCCGCAATGGCCCGGCGGTATACCATCCGGCCGCCACAC
AGCACGCATTCGAACGGGTCACGACTCAGGAACTGTTTCACCATTTGTGCATAGCACACTTTCGCCACT
GGTTCCGGTTTATCCATCCCCAGAGCACGGTACACCTGCGGCAGCTTCTCTCCACACACCCGGTTGGCA
AGGAACCCGAAGTACCTCACCATCTTAAAAAACTTCTCCGGGATGTGCTGTTTCAGCCTCGCGACCAGC
TCACGCTGTGTCAGCGTTTCCGTCGCCGTTTCTCCCGTTTTGTGGTCCAGGTAACGGAAGCTCAGGCTC
GCCCCTCCGTTGTAATGTGCCAGCCGGGAGGCCGCTATCGGCGGCTTCTTCAGATAACGACCCAGGTA
GCGCGCCGTATTCCTCCCGCCGGCCGTTTTTTTCGACATGTACACATGCCAGTATTTTCCTCCGGCTTTCA
GCACCAGGCTTCTCCACTGTGATTCCGTCGTGATATGTGACAACGACTCCGGCATCGCCAGCCCCTCTG
ACCACGCTTTCAGAAGCCGTTGCCGCATATTCCACATCCACCGCGAACGCATCGCGTCTTTCAGGAAGC
TCAGCTTTTTCCACTGACCATGCTTATTCAGACATCCACAGGTTACAGACACATGTACATGCGGATGCCA
GTTGAGACGACGGCCATACGTGTGGATGGCGCAGAAGATACCGGGTTCCTGCCCCCGTTTCCGGGCGG
CATACAGCAGATTCTCCACCGCCAGACGGCACACGTCATTCAGCAGCCACCGGTTGCTTTCGAACACCG
GCCACAGCGTGTCCGGCAGGGTGAAGACCAGATGTACCCAGTCGCAGTCAGGAAGACGATTCAGCTG
TGTCGCTATCCACAGGTCTGTGGCCTTTTTTCCGCAGGACGGGCAGGCACGGCTGCCGCATGAGTTAGT
GACGTATCTGACGTGGGGGCACTCAGGTTTATCGCAGTTGTATTCCTTCACTCCCAGTATCCGCGTGCC
GCAGGCCAGCATTTTTGTGACAGCCTCAACCTCGATATCGCGCAGACCGCCCGCATCCAGGAAGGACG
TCCAGCACTGGTTGGCTGTGAACAGGCGTTTCAGCGGTCGGGGCTGAAAATCACCACCACTTCGGGTC
ATCCGCCTTCATCTCCGCTTCTGTTTCGTATAAATCAAAACGACGGCAGGTATGGCAGAACGTGACGTA
TTCGTACGGTGAATCCGGGTACATTTTGTGGAAGGTTTCCCAGAAACAGACCGTGCAGGAGGGATGAC
CGGTGATAAAGTCTGCAAATACGGACTGATAAGGGTGATTATTGGCTCTGGCGACGGCTTTCAGAACC
TCTTTCACCATTCTGGTGTGGACTTTCTGGTGCTCCAGGTTGTGTGACATGGGAACTCATTCTGGATGGT
TACTCTGAAAGCTCATATTCTGCCACACCCCGATTTGCAGCCGCCAGGCTGCCGTGGTTCAGGTAAACG
CCTGTTCGATAAAGCATTACCCAACGACGAAAACAAACTCAGGTCGCTAATATCTGACCTGAAACAACA
TGGTCAGATACTGCTGGTTGTTGATCAGCCAGCTACCATCGGTGCGTTACCTGTCGCCGTTGCCCGCTC
AGAAGGAGTCCTTGTCGGATACCTCCCTGGACTGGCCATGCGCCGCATAGCCGACTTACACGCCGGTG
AAGCTAAAACTGATGCTCGTGACGCTGCCATCATTGCCGAAGCTGCCCGTACCCTGCCTCACGCGCTAC
GCACGCTGAAACTGGCTGACGAGCAAATCGCCGAACTCTCCATGCTCTGCGGCTTCGATGATGATCTTG
CCGCACAGACAACGCAGGCCAGCAACCGTATCCGCGGCCTTCTGACCCAGATACATCCGGCACTGGAG
CGCGTTCTCGGTCCGAGACTTGAGCACCCGGCGGTACTCGATCTTCTCCAGCGATATCCCTCACCAGAA
AAACTCGCTTCGCTGGGCTTCGCTGGGTGAGAAGAAGCTGGCAGCCCAGCTCTGCAAACTTGCGCCTC
GTCTGGGTAAACGCCTTGCAGCAGACATAGCTCAGGCACTGGCCGAACAAACCGTCGTCGTTCCCGGC
ACGAATGCCGCTGCCGTAGTACTGCCACGTCTGGCACTCCAGCTCATCACGCTGCGTAAGCAAAGAGA
CGAGGTGGCGCTTGAGGTAGAACAGCGAGTTCTTGCTCACCCTCTTTACCCGGTCCTGACCAGTATGCC
CGGAGTCGGTGTCAGGACCGCAGCCAGACTCCTCACCGAGGTCGCCTGCCGCGCCTTCGCCTCTGCCG
CACATCTCGCTGCTTATGCTGGCCTTGCGCCGGTAACTCGGCGATCCGGCTCGTCAATACGCGGTGAGC
ATCCCTCGCGACGGGGTAATAAAGCTCTCAAACGGGCGTTGTTCCTGTCGGCCTTCGCCGCGCTCAGGG
ATCCGCTCTCCAGGGCTTACTACACCCGCAAAATGAGTCAGGGAAAACGACACAATCAGGCGCTTATC
GCCCTGGCGAGACGACGCTGCGACGTTCTGTTCGCCATGATGCGCGACGGGACTTTTTATACCCCGCAG
GGGTCATAACATGCTTGACAACTTAATAGGGGCACCCCCCTAAGCCGTCTATGGGAAGGAGTCTGCTG
TTATTCCAGAGAATGGTAATTCAGTCATATATTTCTCCATCTATGACTTGTCAATACCATCCTGATATTTG
TGGGTCAGCAATATATTCTACTAAAAGGTATTTAAGGTCATTGCGTCCCTGCACTCGAATAATGGTGAA
GAAAGCATGATGAATATATCACGATAACGTTCAGTATGTATCGAGGACAGATGGAGTTACTCCTCTCTG
AAATAGAATTACTGCTCTACTTTTTCTGTTTCCAGAAATTATTAGTAACAAACCCTGATATATTCTTTTGA
AAATGGAGCTTAAACGAGTCTGAAATTAACTTCAGATATTAGAATGCCGTTGGCTTTGTTTTTCAACAAT
TCCTACTTCATTTATCTTTGTCAACCATTGCATTTCTGTAGCAGTGAATTTCAACGTTTTCGTCCGGTTGG
CAGCAGGTATTTGTAATTCGTATATAGCTGCTTTTTTATTGAAAACAGTATATCCATCTGTAGTAACAGT
AGAAGATAATAATACAGGATTTGATACTTCAAGGATGATATTTTCTTGTTTTTGTGGAAGGCAAGGGAA
TATAGTTTGTGTAAGCATATTGCTTCCTTATGTTATATTTTAGTAAAGAATTTATTTTTTACATTATAGTCG
GCTAAATGAAGTTTACTGTAAAAAAACGATACTTTTTTCGTAAGCGATGAGATGTGGAGCCAGAAAAAT
GGCAGCGTTACGTCATTGAACTGTCCTTGACCCACGATATTCAGACAGTCGTTGTATCTTAAGTTGACAT
TACCCGCTGACGTCGATATTCCCTTGGTGAGAGATAACCCAACGCACTATGCGGATGGTTTTCATTGTA
ATGCTCGATCGCCACTGCAAGGTTATGCAATGCCGTTCTTGGTTTCGGCATGAACGCGATGCAGTCTTC
CTTCATCGTTTTCACGAACCGTTCTGCTATTCCATTACTCTGAGGGCTACTGACTGCCGTCGTGCAGGGC
TCCAGATTCAACTCTCTGGCGAACTGCCGTGTTTCATGAGCCGTATAAGCCGAACCGTTATCGGCCACT
GGATTGGCCGTTCCGGTCCTTTATCGCCATATCGCTGACATGACATCCTGCACCGTTGCTTTGTCATAAT
CTCCCGTGCCTGCTGCCCAGTCTATGGCTTCACGATCGCAATATGGAACGTAACGCTGTCATCGGGAGA
ATCGTCAATGTGGCATTCGGTTAACGCTGATATGACTTTCCGTGTCAAACCGGAATCTCTAATTTTGACG
ATTTTCTTTTTAGGTTTAATTCCCTGAGGGCGACGTAGCAGTAAATCCGACGGTGAATCACGCTGATATT
CGCGGGTTGGATACCGCATTACAGAGGTCCGTCATATGAGCCTGCCACTTTCTTACGTCGCGAATCAGC
TTTGTTCCGCCGTCGCCAAGCGCTAACGTGCGTTACTCTTGTACCGGGCTACGTCGCCTTCAGGGAACT
GGTTATTCTCATTTTTTATTTGTGACATTGTGGTTGTTGTGCGACAGACATTGCTATTCGGCCCATATCCC
AGATAAAACCCGCCAGCTCACGTGCAACAGCAACAATTGTAATATTGACATTCTTTCCTTTGGCCTGAA
GTTTTCGATACCTCTTACAAAGCCTGAGTTGAGCATCCCATGCTCTGTCAATGACGGGGCGGGGTAAAT
TTTCCTGCCTTTTTTGTATTGCCGGACTGATGCGTGCCGGATGACGGTAACTCCACGCTGCCTCGACCAA
TAGCTTTCTGGCATAGCTGTTTCCGGCTTTGGTAATGCTGCCCTGATGTCTGCTTCCACCCGATGAATAT
TCTGATGGCGTGATACCAAACCATGACATCAGTTGTCGTGGATGTTCAAAACGGGTCAGATCACCCAGT
TCAGAAATTAATCCAACCGCAGTAATAAACTGAATCCCTCGCATGGCTTGCAGCGCCTCTACTACCGGA
TACAGCCTCCATTCGGTAACGGCCTCCTTCAGGGCGGATTCCAGCCGTTCACATTGTGCCTGCCGATCTT
CTATTGTACGACGGTGTTCGTCAAAAGCTAATTGTCGCCAGGGACTTTCGAATGAGTATTTGCTGAGCC
AGCGTCGGTGTGCTGGGCCCCAATCTGCTCGCCCGACATAATGGACCCCATGAACCAGAAGAAACGAT
TTCAGACGTTGCCTTGCATGCCGTAAATCATCGCGGGCAGATGCCCATGCCCGGGCCAAATCCCGGAAT
GCCTCATCTTCAATGCCGGGTACGTAGACAGCAGAAAGATCTCCTGCACGCAGTGAACGCACAAGTCT
GATGGCATCACGGCGATCGGTTTTAACCCGCTCCCCCGGTTTTTTCGGGATAAGTGAGGGGGCGCAGA
CCATGCAGTCAAAACCGGATTTCACCAGCCGACGATAAAGTCCATAACCACAGGGCCCCGCTTCATAAA
CGATACTGACCTGCGATGACTTTGACCTGAGACGTTTACAAAGATTCTGAATATCAGTAGGTGATGTGC
CAATTTTACCCATCAGTTCAACAGGTTCTGAATTGATAGCATAAGCGACAGTAATCGATTCTTTGTGAAC
ATCTAACCCGACGTACAATGTGTTATTGTTGTTCATGCTGGCCTCCGTGGTGGAAATACCGGGGTGTAG
CAACAGCTCACCCATGCGGCTCTGACAACGATGTTAACCCGCGTTAAATATCCCACGTTGGCCAGCATC
TGTCTCACGGAAAGTCATACTGCCTTACCAATATCAACACCAACCGGATTTGTGGAGCCTCCAGTCTGA
AGGGGCTTTATGCGTTCCGTATAGGATATTATGATGCTGGAGTTTTGCGAAGCTCCACTCCTTTCCGAA
CATTTACACAGATATTGCCTAAGAAAAGTGAAACCATAATATGGTTATAGTCCCTTTCAGTGCAAATACT
TAGCTTGTTGCACAGAGAAATAGAAGCTGCATAAGCTCTTTCTTCAAAAAATGTAAATAAAGTTAAATA
TAGGAAAAATTACTTAATCTATCTTAATAACGGAAAGATTTTGTATACAATCACTGTTACAGAAATTTCT
TAGTTACTCTGTAAACACTAAATATAGTTTGGTATATTCTGTTGAATTTATGATGGATATGGGACATAAA
AACAAAATAGATATAAAGGTTCGCTTGCATAACTATATTATTTTATATGCAAAAAGGTGTTCAATGACG
GTTAGCTCAGGCAATGAAACTTTGACTATCGATGAAGGGCAAATTGCTTTTATAGAGCGAAATATACAA
ATAAACGTCTCCATAAAAAAATCTGATAGCATTAATCCATTTGAGATTATAAGCCTTGACAGAAATTTAT
TATTAAGCATTATTAGAATAATGGAACCAATTTATTCATTTCAACACTCCTATTCTGAGGAGAAAAGGG
GGTTAAACAAAAAAATATTCCTCCTCTCTGAGGAGGAGGTTTCTATCGATTTGTTCAAATCTATAAAAGA
GATGCCTTTCGGCAAAAGAAAGATCTATAGTTTAGCTTGCCTTTTATCAGCTGTTTCTGATGAGGAAGCT
TTATATACTTCGATATCGATAGCTTCTTCTCTTAGTTTTTCTGATCAGATAAGGAAGATTGTTGAAAAAA
ACATCGAGAAGAGATGGCGTCTTTCTGATATTTCAAATAACTTGAATTTATCAGAAATAGCTGTTAGAA
AACGATTGGAGAGTGAAAAATTAACATTTCAACAAATCCTTCTTGATATTCGCATGCATCATGCAGCAA
AGCTTTTATTGAATAGTCAAAGCTATATTAATGATGTATCAAGACTTATCGGAATATCAAGCCCATCTTA
TTTTATAAGGAAATTTAATGAATATTATGGTATAACTCCAAAGAAATTTTACTTATATCATAAAAAATTTT
AAATGCTTCATAGCCCATCGCTATTGCCAGATGGGTTTTCCCCACACCTGATGGCCCCAGTAATACGATA
TTTTCATTACGTTCACGTATAGGAAATTGAAAAACTAAGCGATACCCCTGTCCGCGCCTTATGCCACGTG
AGCCTTTCTGTTTTTCCCCACCACTCCGGTGTTTTCTCTCTCTGCATGGTCGATTCTTATACCGCAACAGG
CTGTTTTTTATACTTGCACCGATTCCCGGGCACACTTAGCCTGTGTCAGGCCGGCATATACCTCAGCAGA
CTGATATCCCGCGCGTTTTTCTTCAGCCCCTCCACATTCAGCCCCGCAATGGCCCGGCGGTATACCATCC
GGCCGCCACACAGCACGCATTCGAACGGGTCACGACTCAGGAACTGTTTCACCATTTGTGCATAGCACA
CTTTCGCCACTGGTTCCGGTTTATCCATCCCCAGAGCACGGTACACCTGCGGCAGCTTCTCTCCACACAC
CCGGTTGGCAAGGAACCCGAAGTACCTCACCATCTTAAAAAACTTCTCCGGGATGTGCTGTTTCAGCCT
CGCGACCAGCTCACGCTGTGTCAGCGTTTCCGTCGCCGTTTCTCCCGTTTTGTGGTCCAGGTAACGGAA
GCTCAGGCTCGCCCCTCCGTTGTAATGTGCCAGCCGGGAGGCCGCTATCGGCGGCTTCTTCAGATAACG
ACCCAGGTAGCGCGCCGTATTCCTCCCGCCGGCCGTTTTTTTCGACATGTACACATGCCAGTATTTTCCT
CCGGCTTTCAGCACCAGGCTTCTCCACTGTGATTCCGTCGTGATATGTGACAACGACTCCGGCATCGCC
AGCCCCTCTGACCACGCTTTCAGAAGCCGTTGCCGCATATTCCACATCCACCGCGAACGCATCGCGTCTT
TCAGGAAGCTCAGCTTTTTCCACTGACCATGCTTATTCAGACATCCACAGGTTACAGACACATGTACATG
CGGATGCCAGTTGAGACGACGGCCATACGTGTGGATGGCGCAGAAGATACCGGGTTCCTGCCCCCGTT
TCCGGGCGGCATACAGCAGATTCTCCACCGCCAGACGGCACACGTCATTCAGCAGCCACCGGTTGCTTT
CGAACACCGGCCACAGCGTGTCCGGCAGGGTGAAGACCAGATGTACCCAGTCGCAGTCAGGAAGACG
ATTCAGCTGTGTCGCTATCCACAGGTCTGTGGCCTTTTTTCCGCAGGACGGGCAGGCACGGCTGCCGCA
TGAGTTAGTGACGTATCTGACGTGGGGGCACTCAGGTTTATCGCAGTTGTATTCCTTCACTCCCAGTATC
CGCGTGCCGCAGGCCAGCATTTTTGTGACAGCCTCAACCTCGATATCGCGCAGACCGCCCGCATCCAGG
AAGGACGTCCAGCACTGGTTGGCCGTGAACAGACGTTTCAGCGGGGGGGGAGTAAAAGCGGACAACA
TGAGGACGGATTAACCCCCGGTATCCTGTAGCCGGGTAAGGGCTGCTGTGTCGCCACGGCGCTCCAGG
ATGGAGGCCGCGACCATCGCAGGTGGTGGGTCCATGGGTTTGAGCATCTTCATCCAGTCCCATTCACGT
TGTGAGAGCTTCTCAGCACCAACAGAGAGCAGAACGGTATCTATACTTTTGCGGGTCAACATAAGAGG
ATGAGTCCTTAATAAAACAGGTCCTCAGAGCATACATATTCTGCGGAGCGTGCGGCAACAACAGGACG
AACAGGGGCGGTTTTCAATTTGCAGCCGTCAGGCTGCCGTGGTTCAGCGCCTGATGGGAATCCTCTCAC
TTAAGGCAGCGATTAAGGTCAAGCGATACCGCTCTTACAGAGGAGAGGTAGGGCAAACCGCCCCCAAT
GTTCTCCAAAGAGATTTCAAGGCTACGCGGCCAAACGAGAAGTGGGTTACCGATGTTACTGAATTTGC
AGTCAATGGGCGCAAGCTGTATTTGTCTCCAGTAATAGATCTCTTCAACAACGAAGTTATTTCTTACAGC
CTTTCGGAAAGACCAGTGATGAACATGGTCGAGAATATGCTCGATCAGGCATTCAAAAAGCTTAATGA
ACCGCCCCGGGAATCCTGGAGACTAAACTTCCTGAGAAAGAGGTAAACAGGATGACTAAAAATACTCG
TTTTTCCCCCGAAGTCCGTCAACGGGCAGTCCGTATGGTTCTGGAAAGTCAGGGCGAATATGACTCACA
ATGGGCGACAATTTGTTCCATTGCTCCAAAGATTGGCTGTACGCCGGAGACTCTGCGTGTCCGGGTTCG
CCAGCATGAGCGGGATACCGGGGGCGGTGATGGAGGGCTCACCACCGCTGAACGTCAGCGTCTGAAA
GAGCTGGAGCGTGAAAATCGTGAACTGCGCCGCAGTAACGATATCCTTCGCCAGGCTTCCGCTTATTTT
GCGAAGGCGGAGTTTGACCGCCTCTGGAAAAAATGATGCCACTGCTGGATAAGCTGCGTGAGCAGTAC
GGGGTCGGACCGCTATGCAGCGAACTGCATATTGCCCCGTCAACGTATTACCACTGTCAGCAACAGCG
ACATCATCCGGATAAACGCAGTGCCCGTGCGCAGCGCGATGACTGGCTGAAGAAAGAGATACAGCGC
GTATACGATGAAAATCACAAGGTATACGGTGTGCGTAAAGTCTGGCGTCAGTTGTTACGGGAAGGTAT
CAGAGTGGCCAGATGCACTGTGGCACGTCTCATGGCGGTTATGGGACTTGCCGGTGTTCTCCGGGGTA
AAAAGGTCCGTACGACCATCAGCCGGAAAGCCGTTGCCGCAGGCGACCGCGTAAACCGTCAGTTCGTG
GCAGAACGACCTGACCAGCTGTGGGTGGCTGATTTTACTTACGTCAGCACATGGCAGGGCTTCGTCTAT
GTGGCGTTCATCATTGATGTGTTTGCCGGATACATCGTGGGGGGCGGGTCTCATCGTCCATGGAAAC
GACATTCGTGCTGGATGCACTGGAGCAGGCGTTATGGGCCCGTCGACCGTCCGGCACGGTCCATCACA
GTGATAAAGGTTCTCAGTATGTATCGCTGGCCTACACACAGCGGCTTAAGGAAGCCGGATTACTGGCA
TCAACAGGAAGTACAGGCGACTCGTATGACAACGCGATGGCGGAGAGCATCAATGGTCTTTACAAAGC
GAAGGTAATACACCGTAAGAGCTGGAAAAACCGTGCAGAAGTGGAACTGGCCACACTCACGTGGGTG
GACTGGTATAACAATCGACGATTGCTGGAAAGGCTGGGCCATACTCCTCCGGCAGAAGCAGAAAAAGC
TTATTATGCTTCCATCGGAAACGATGATCTGGCAGCCTGAGTTCACAGATAAAACACTCTCCAGGAAAC
CCGGGGCGGTTCAGTTGCTGAACTGCTGGAACCGCTGTATGACATCCTGCGCCAGTATGTGCTGATGC
CCGGTAAAGTCCATGCCGATGATATCCCCGTTCCGGTCCAGGAGCCGGGCAGCGGTAAAACAAGGACT
GCCCGGCTGTGGGTCTACGTTCGTGATGACCGTAACGCCGGTTCGGAAATTGCAGCATAAGTTATCCAT
GCTCTGAGGTGGGTGGAGGTGGGCCCTCCGGTCATGTCAACGCCACGATGTTTGACCGTTATTTGCCAT
TTTCATGCCCGCTAAATACTTTCGTTACTGATGCTGTCCGCTTTCCACCATTTCATCATCACTGATTGGCG
TTGTGTTTTTGGTCAGCACTCCAGCTTTACGTTTCTCTTTTAACCGGTAACTCTCTCCTTTGATATTCAGCG
TGGTTGAGTGATGTAGCAGCCGATCCAGGATCGCTGTTGCCAGTACGTGATCTCCGAACATTTCTCCCC
AGTCTGCGAACCCTTTGTTTGACGTCAGTATGATGCTCGCTTTTTCATATCGACGGTTCAGTAGCCGGAA
GAACAGACTGGCTTCCTCTCTGTTCATCGGCAGATAGCCTATTTCATCCAGGATCAACACCCGGGCATA
ACTCAGTTGCTGTAGCTGACGCTCCAGCCGATTTTCCTGTTTCGCTTTCATCAGTGTCGCGATCAGTCTG
TCCAGTGGCATAAACAGTACCCGATGTCCCGCATCCACCGCTTTCACGCCAAGAGCTATGGCCAGATGA
GTTTTTCCGACACCAGGAGGGCCCAGCAGGATCACGTTTTCGCTGCGCTCCACGAACGCCAGACCAGCC
AGTTCCCGGACAACCTTACGGTCGATGCCCGGCTGGAAGGTAAAGTCGAACTGCTCCAGCGTTTTGACC
CACGGCAGACGAGCCTGCTTCAGCCTGGACTCCATACCGCGCTGATGCCTGCCGTTCCATTCCTGCTGT
AGCGCCATGCACAGGAACTCCCGGTAGTTCAGCTCTTTTTTAGCTGCCTGTTCCAGCAGGCTTTCAACGT
GATAACTCAGATGCTCCATTTTCAGGCGACTCAGCAGGACTTCCAGTTCATGCATCACAACAGCTCCTCA
TAGGCACTCAGTGGTCGATGTTCCACCTGACTGACCTGCTGCCAGAGCGGGGCGTGATGCTCCGGCAC
TGTCTGCCAGCCAGACGATGCTGAACAGAGGCGATGTGAGGCCACCAGTTTCTCATTACTGTAGATCCG
CAACTCGTCATCCAGCGATATTCGTATCGATACCGGCTGACCACACAGCGCTTCGGGAACGCTGTAACG
ATTACCACCAACCTCGATATAGCTGTCCCAGGACACATGGCGGATATCGAAGTAACTGGTATCGAAGTC
TGTATCCGGTAACGGCTGCAGATGTTCCTGCTCCAGCGCGAAGCGCTGTTCCGGCGTTTCTTTGAACTG
GCGAAGCTCCCGTTTGTCAGCCACATCGGCTATCCATTGCTCCAGTTGTTGATTGACATGAGTGAAGCT
GTCGAACCTGCGGTACCGGACGAAGAAGTTCTCCTTGAGGTATTTCACCATCCGCTCAACCTTACCTTTT
GTTCTGGCCCTGCGTGGACGGCATGCCCGTGGCAGGAAGTTATAGTGGTCAGCCAGCAACAGGAATCC
GGAGTTGAACACGACTTTCCCGTTGTTATTCTTCAGCACCGCAGCCTTCTGGTTATCAACCAGCACCGTT
TTCACACAACCACCGAAGTAGCGGAAGGCGCGAACCAGTGATTCGTAGGTATGCTCAGCATCCTGTTTT
GGTGCGGCGAAGACATGGAAGCTGCGGGAGAACCCCAGCGTATTAACCGCAAAGTTAACTTTGCACC
GTTGCCCGGCAACCTCCACCTCAACTTCGCCCCAGTCATGCTGGAGCTGGTATCCAGGCTGAGTTTCGA
AGCGAACTGTTCTTTTTGACGGACGCATTTTACGTTTGGGCTGGATGTAGTAACGCAGCATGGAACGTC
CGCCCGTATAACCCATTGCCTTAATCTCCGCGAAGATAACCTCGCTATTCCAGACATTCTCTGCCAGGCG
CATGTCGATGTAATCCATAAACGGTTTCAGCTTAACCATTTTGTGGGGGTCTTTCTGGCTGGCGGTTCA
GGGTATTTGAGGTAGCGTCTGACCGTCCGTTCAGAGCAACCAATCTGAGTCGCAATATCGACGATGTAC
GCGCCCTGCTGGCGCATTTGCTTTATCATGTAAAAGTCCTCTCTGCTCAGCATGTTGATGTCCTTTCTGG
TGTGAGAACCTTAAGGAAACAACATGTTGGGTGGAGCGGACAATCCAAATGGCGAATTACCGTCTTAT
ATCACTGGCGCTGACAGTCAGGGTTTACAGGAGCACTGACCAAACGACCCGGTGCTGCTTCATTCTAAG
GAATGAGGTGGTGAGCGACTCGGGAAAAGGCAGAGATCTACTGTCACGTGGGGTTCGGGAAGCTGAA
CGAAAGTAAACTGTCCGATGAAGCATCGAAATCTTAGATGATGTCAAAATGGGGCCGTACAGCCGCCC
CCTGATAAGCGCAGAAGACACCTGCTTACTGACTGCGCGGCATCCGGTGTGAAGGCAGCAGGATCCCG
GGCCAGACCATCTCAGGGAACGTGAGAACCTGTTTCCGGCACGTATCGGGAAACAGGGGCGGACCCA
TTCGTAGTAGTGATGAAAGATCTGTAATGGGTCTGGAGCGAAGGGATGGGGATCACATTCATTGCAGA
AGACACAACCCAAAAGGGGATGACGTAACTGATGCAACGAAAATCATTTGAAATACCAAAAGCACTGG
TCTGGGCATCATATCTGGATGTGCGTCGCAACAAGGGAGCCCCCGGATGCGACGGGCAGACTTTGAAA
ATGTTCGACCAGCAACGTGATGGCAATCTATACAAGATCTGGAACCGGCTATGTTCGGGAACATGGTTT
CCGCCACCAGTGCTGGAAAAACGGATCCCGAAGTCGAATGGCAAGGAACGCATTCTGGGGATCCCAAC
AGTATCCGATCGAATTGCTCAGGGAGCGATAAAACTTTTCATGGAAGAAAAGCTTGACCCGATTTTCCA
TGCTGATTCATATGGTTACCGACCAGGCAAATCAGCTCATGACGCTCTGAAACAATGTGCCATCCGGTG
CTGGCGTTATAGCTGGATACTGGAAGTGGACATCAGTGCGTTTTTCGACCATGTGAGACATGATCTGGT
GCTCAAAGCTCTGGAACACCACGGGATGCCTAAATGGGTCATCCTGTACTGTCGACGCTGGATGGAAG
CGCCAATGCAGAGTTGTGAAAATGGAGAATTAATAACCCGAACACGAGGCACTCCGCAGGGGGGGGT
CATTAGTCCGCTACTTGCTAACTTGTTCCATCACTATGCATTTGATTTGTGGATGGAAAGAGAATATCGG
GGGTACCGTTTGAGAGGTACGCTGACGATATTGTAGTACATTGTTCACGAATGAGTGATGCAACAAGG
CTGAAGAACAGGTTATCGGAGCGCTTCTCGGAAGTCGGGCTGGTCCTGAATGCAGGGAAAACGAATAT
CGCCTACATTGACACGTTTAAAAGGAGAAACGTCGCAACGAGTTTCACCTTTCTCGGATATGACTTCAA
AGTGCGTACGCTGAAGAATTTCAAAGGCGAACGGTACCGAAAATGCATGCCGGGTGCGTCAAATGCA
GCAATGCGCAAAATAACAGAAACAATCAAGAAGTGGCGTATACATCGCTCAACAGCTGAGAGTTTGCT
GGATTTTGCGAGGCGCTACAATGCGATAGTGAGAGGCTGGATCGAGTACTACGGAAAGTTCTGGTCCA
GAAATTTCAACTATCGACTGTGGAGTGCAATGCAGTCACGTCTGCTCAAGTGGATGCAGTCTAAATACA
GACTTTCGAACCGGAAGGCTCAGCGAAAGCTGACGCTGGTAAGGAAGGAGTATCCGAAGCTATTTGTT
CACTGGTATTTACTACGTGCATCAAATGAGTGATCAAGAGCCGTATGACGCGAGAGTGTCACGTACGG
TTCCGTGAGCAGCCGGGGAGTGAAATTCTCTCCGGCTGACTCGACTCGCGGGTTCCGACAGCGATGAC
GAACATGCGGCGGTGTTGTACTCGCTGATCGGCACATGCCGTCTGAACAATGTGGAACCAGAAAAATG
GCTGCGTTACGTCATTGAGCATATCCAGGACTGGCTGGCAAACCGGGTACGTGATCTGTTGCCCTGGA
AAGTTGATCTGAGCTCTCAGTAAATATCAATACGGTTCTGACGAGCCGCTTACTTTTGGCGTAAATCTGC
TGCGCCTTTACTGAACACAATGCCGTATTCAGCAAGAAGTCCACGAATGTGATTTATCAGTTGTGTCCTT
TCCATTGAAACCAACCGACGCATATTCAAAAAAGTCTGGACGTCCTGTTGCTCCAGAGTTTTTATTGGTA
CGGGTCGTATAGTTCCATGTTGGCAGGCCTCAGCTATTGCCTGTGCGTCGTTATAATCATTTTTCTGGCA
CCGCTGATAAGCTTTAACATGTTGTGCCGGAAGCAATATTACATCATGGTCGAGTTTCCTGATTTCCCGG
GCCCAGTAATGAGATGCCCCGCCAGCCTCCATCGCAACAGTCATTTTAGGCTGTTGCACCAGCCAGTTA
AGCAGTTGATTGCGAGAAAGTTTGGTCTTTCCTGCGGGTTTGCCATGAGCATTGATACTGAAAAGATAA
AAGTTCGTTTTAGCCAGATCAATGCCGGTGATTTTCTGTTCAGACATGGTATCCCGCTTACTTTTGTGGT
TAAAGTTTATCCATCATGGCGCAATTGAAGGCTGCCGTGAAAGAGGGGGAAGACTATCTCATCGGTTA
CTGTTAAACGGCTAAGAGAAACTATCTTTTTCAAGCAAAGAAGATACGGTGAAACAGGCGATTTCACC
GTGAAATACTGTTCAAAAAAAACGCTAATAAAAGCAAAATTTCTTTTAAAACAGCCAATTAAAAACTGG
TGAGTTGCTGGCGAAAAACTTGAATTTTCACAGCGAATTCAGATTATCTTTTAGTACAAGAGCAATCAT
ACGATCTAACTTATCCTGAACTTCCAACGGTAATCTGCCGAATTCATATGAGAACGTACGTCCCTTTACT
CGTTTTCGAGCAAATTTATCTTTACTATCAAATTTCCATAACAAAGTTACAACCGCTTTATCCTTTACTCCT
GAATCAAGCAATGAAGACATTTCCTTGGTTATAAGCCGCATAATACTGTTTTTAACCTCGTCTTCAGAAA
GATTGTCATCAGCAGTAATAAGCTCAATACTCGGCGAGATATTCTTCACAACTTCATCAATAGATATATC
ACCCAGCCTCAGTCGTTCACTTAATTCAGCCAGTTGCCTGTAATCGGCGTATGTCAGCTCAGACTGAACA
GGAAAAAGAGACACAAAATCTTTAGGTACGGACGCTGCTTGGAGGGCACGAGTCACCTTAGCTGCCGA
CAATCCCTCTCTTTCCGCAATTTGCTTCTGGTTCATACCGGCCTCTTTGAGACGTACTAAACGCAACCCAA
TTTCACGAATATTATGTTCAAGTGACGTCTGCAAATCTTTCGCCAGATGCTGAGCTTCAGAAACAGTAA
GCTCATCATCCGTTACAAGTACACGTAATCCAACCTTGCATAGCAAAGCTGCTGCCCGACGACGGGAAC
CATCCAAAATCTCAATAAGATCGCCAGTCCGAATCCCAATACATGGATAGAATTGTTGTAAATGTATAG
TTCTGGTGATGTCTITTAGTGAGTCCGGCGTCAGGGCTGTCTGTTCGCGGCCATTCACCTCTTCAACCAC
AAAAGTTTGAGTGTCCACTTTGTCATGATCAACCTTGATCTCAGTAAATTTTGCCTTTCGGCCAGTATTG
AGGGTAAATACATGAACTGGAGCCGATATTTCTTCAGTATTATTCAGGATATTTGTATTCAGCGTACGG
CCAATAGTCGGTCGGTGCTTTCTGTTTTCCATCAGTAGTTGGCCCTTATAAATTCAATACGATCAAAAAC
TGCTTTAGCGAAATCTTCTGCTGCCATACGTGCATTTTTCAAAGCTTCACCACTGCCAACATAAGTAACA
GGATTCGCAGATATCACGGTATCAAACGACTCGCCGCTTCTCTCAAAACCATCAAGACGTGGCATTGAA
ACATCAAGCATGTCACCACCGAAAATCTCTTTTGTCAGGCTATGGCAGTATTTATGATCTGACTTATTCG
CCAGTTTCGACATGAAGCCAATATTTGCCTGCAAACGGCATGAACATCCATCCTGTTCAATAATCTGTAC
AAGTTCAGGTAAACGAGCCAAATATTTAAGCGTGGAATGGAAATCAACTTGTGCTGGCGGCACCGGTG
TAAACATGATATCTGCGGCAGCAATGGCATTTTTCAAAAATGCGTCCAGATGCGGGCCTGTATCAATAA
GGATGAAGTCGAAATCATGCTTCAGCTTATCAATAATATTCTCACGCAATATCGCGTGTTTGTTTTGTCC
TAATAGATGCTCCTCGCACAGAGTATCCCAGTTTGATGCAATAAAGGCGTCGTCAATAGAAGCCGGCAT
AACATAAACGCCAGGGATAACTGACGGAACGATAAAATCTTCCAGTAGTTCCTCACGACTCACGTTCTG
CAACATAGCCTGCGGTGCGGTTGTATCAACCAGCCCCACCGCATGCAAGTAGTTCAGAAACATAGTGG
CTGATGACTGGGGATCTAAATCAAGGAGCAGAATACGTAAATCTTCTGACAGCAGGTGAGGATGAGCA
CGCAGGGCGTGAGCTACGGAAACACTACTCACGGTTTTAGACACACCGCCTTTTAGACTACCGATGAAT
ATGGAATATGCCTCACTATAACGATCGCGATATTTAGGAATGCCACGATGTGCATAGATATCGATAATG
TTCTGGATGGTTAATGCATATTTTGTCGCGGTTCCTGCTGGGCGTTTTTCAAACTGATAGCCCTGAGCCT
CCATCTCACCGACTGCACCATCCACGCTTGCGCGCGTTAATTTTGGAAGCTTAGCTACAGCAGACTTTGA
ATAGACTTGATAAAAAACATCAGCCTGAAACTCCTGCTTTTGCTCCTGAATCTGTTTTGTTAGGGCGAGC
AACATTTTATCTGCACGCTGCGCAACCTTACTTAACTGCTCAAAACTAGTCATTCCCCCTCCGTGCTATGT
AGGATTTTAACTTATTACAAAAAAAACTGCATTAGCAGGGTAATGCATGCAAGTACATATTGTACATGC
TTATATTAAAAATGCGCGCATTGCTGATTTATTAATCATCGGAATGAATATTACTGTAAGATAGTATGCC
TTTCCGGGAGAAAGACGCTGATTTCCCCCGAATAATCCAGTTCACCACCACACTCCGGACAACAGCTTT
CTTCAGGCTCCAGATGGTGTATTTCGCGGGAAACATGTTCCGGTAACGGACGGCGATGACGTGACTGT
CGCAACAGGCGGGGGTAGTGCTTCCAATCAGTAGAACATGTGCTTTTCAATGAAGGAGCCGATGACTT
TTTCGTGGATTTCCACGGAACGCGAGAAGCAGATTGTTTTACGTGCCAGCCGCTTGATGCGGGTGCGG
AGGGTCCGGTTGTTACGTTCAATACGTTGCGTGAAGATTTTGCCGGTCAGGTGTTTCTCCTTCGGCACTT
CTCTGGCGTAGCTGCCCCAGTCATCGCTGGTCACCATGCCGATACAGAACGGAGTCAACAGGGCTAAC
AGTTCCCGACAGGTCTCATCATTCCGTGGCCCGAAAGTCGAAAGTGTAAGCCAACACACCGCCAGTTTT
AGTGTTGTACGCATACCAGAGCCAGTGTTGTGTCAACGACGGATGAAAAGTGATCCACTTATATCTCCA
CCAACGGCCCAATATTGATCCACCGTTTTACTCAGGATTAGCTTCTGCTATAACCCCGGCCTTTCGTTTCT
GTCTGAGTCGATAGCTTTCTCCTTTGATTTGAACGAAATAATCCGACGGTGAATCACGCTGATATTCGCG
GGTTGGATACCGCATTACAGAGGTCCGTCATATGAGCCTGCCACTTTCTTACGTCGCGAATCAGCTTTG
TTCCGCCGTCGCCAAGCGCTAACGTGCGTTACTCTTGTACCGGGCTACGTCGCCTTCAGGGAACTGGTT
ATTCTCATTTTTTATTTGTGACATTGTGGTTGTTGTGCGACAGACATTGCTATTCGGCCCATATCCCAGAT
AAAACCCGCCAGCTCACGTGCAACAGCAACAATTGTAATATTGACATTCTTTCCTTTGGCCTGAAGTTTT
CGATACCTCTTACAAAGCCTGAGTTGAGCATCCCATGCTCTGTCAATGACGGGGGGGGTAAATTTTCC
TGCCTTTTTTGTATTGCCGGACTGATGCGTGCCGGATGACGGTAACTCCACGCTGCCTCGACCAATAGC
TTTCTGGCATAGCTGTTTCCGGCTTTGGTAATGCTGCCCTGATGTCTGCTTCCACCCGATGAATATTCTG
ATGGCGTGATACCAAACCATGACATCAGTTGTCGTGGATGTTCAAAACGGGTCAGATCACCCAGTTCAG
AAATTAATCCAACCGCAGTAATAAACTGAATCCCTCGCATGGCTTGCAGCGCCTCTACTACCGGATACA
GCCTCCATTCGGTAACGGCCTCCTTCAGGGCGGATTCCAGCCGTTCACATTGTGCCTGCCGATCTTCTAT
TGTACGACGGTGTTCGTCAAAAGCTAATTGTCGCCAGGGACTTTCGAATGAGTATTTGCTGAGCCAGCG
TCGGTGTGCTGGGCCCCAATCTGCTCGCCCGACATAATGGACCCCATGAACCAGAAGAAACGATTTCA
GACGTTGCCTTGCATGCCGTAAATCATCGCGGGCAGATGCCCATGCCCGGGCCAAATCCCGGAATGCC
TCATCTTCAATGCCGGGTACGTAGACAGCAGAAAGATCTCCTGCACGCAGTGAACGCACAAGTCTGAT
GGCATCACGGCGATCGGTTTTAACCCGCTCCCCCGGTTTTTTCGGGATAAGTGAGGGGGCGCAGACCA
TGCAGTCAAAACCGGATTTCACCAGCCGACGATAAAGTCCATAACCACAGGGCCCCGCTTCATAAACGA
TACTGACCTGCGATGACTTTGACCTGAGACGTTTACAAAGATTCTGAATATCAGTAGGTGATGTGCCAA
TTTTACCCATCAGTTCAACAGGTTCTGAATTGATAGCATAAGCGACAGTAATCGATTCTTTGTGAACATC
TAACCCGACGTACAATGTGTTATTGTTGTTCATGCTGGCCTCCGTGGTGGAAATACCGGGGTGTAGCAA
CAGCTCACCCATGCGGCTCTGACAACGATGTTAACCCGCGTTAAATATCCCACGTTGGCCAGCATCTGT
CTCACGGAAAGTCATACTGCCTTATAAGGCTTTCCAGTTGCAACTGCCCGGCGAGCGCCATCAGTCGTT
GATGTTGCAGTTCCATCATCACGCCACTCCTCTGCAGAATGAGTCGTAGATGGAGAGTGGATGATGCTG
GGGGGTGCCCCTATTAAGTTGTCAAGCATGTTATGACCCCTGCGGGGTATAAAAAGTCCCGTCGCGCAT
CATGGCGAACAGAACGTCGCAGCGTCGTCTCGCCAGGGCGATAAGCGCCTGATTGTGTCGTTTTCCCTG
ACTCATTTTGCGGGTGTAGTAAGCCCTGGAGAGCGGATCCCTGAGCGCGGCGAAGGCCGACAGGAAC
AACGCCCGTTTGAGAGCTTTATTACCCCGTCGCGAGGGATGCTCACCGCGTATTGACGAGCCGGATCGC
CGAGTTACCGGCGCAAGGCCAGCATAAGCAGCGAGATGTGCGGCAGAGGCGAAGGCGCGGCAGGCG
ACCTCGGTGAGGAGTCTGGCTGCGGTCCTGACACCGACTCCGGGCATACTGGTCAGGACCGGGTAAAG
AGGGTGAGCAAGAACTCGCTGTTCTACCTCAAGCGCCACCTCGTCTCTTTGCTTACGCAGCGTGATGAG
CTGGAGTGCCAGACGTGGCAGTACTACGGCAGCGGCATTCGTGCCGGGAACGACGACGGTTTGTTCG
GCCAGTGCCTGAGCTATGTCTGCTGCAAGGCGTTTACCCAGACGAGGCGCAAGTTTGCAGAGCTGGGC
TGCCAGCTTCTTCTCACCCAGCGAAGCCCAGCGAAGCGAGTTTTTCTGGTGAGGGATATCGCTGGAGA
AGATCGAGTACCGCCGGGTGCTCAAGTCTCGGACCGAGAACGCGCTCCAGTGCCGGATGTATCTGGGT
CAGAAGGCCGCGGATACGGTTGCTGGCCTGCGTTGTCTGTGCGGCAAGATCATCATCGAAGCCGCAGA
GCATGGAGAGTTCGGCGATTTGCTCGTCAGCCAGTTTCAGCGTGCGTAGCGCGTGAGGCAGGGTACG
GGCAGCTTCGGCAATGATGGCAGCGTCACGAGCATCAGTTTTAGCTTCACCGGCGTGTAAGTCGGCTA
TGCGGCGCATGGCCAGTCCAGGGAGGTATCCGACAAGGACTCCTTCTGAGCGGGCAACGGCGACAGG
TAACGCACCGATGGTAGCTGGCTGATCAACAACCAGCAGTATCTGACCATGTTGTTTCAGGTCAGATAT
TAGCGACCTGAGTTTGTTTTCGTCGTTGGGTAATGCTTTATCGAACAGGCGTTTACCTGAACGATTAATG
GCAACAGCGTGATGCGTATCTTTACCGACATCAACGCCGATAAAGACCTGGACGGATTCGTAATCGCTG
GATTCGGTCATTCTGTCTCCCTTGTATATGGGTTAACCAGATAACCACGGGGAGCAGGTACCGGCATCC
ACGTTACAGACGGTCCCGGCAAAAGTGCCTGACCTGACCCCTATTAGCGGTTACCAGCGCCCCACCAGA
CCCGGTGACATCACCCCCCGGATCATGGACGACTGGGGGCAGTAATCATGCCGGGTCTGGCTGGCTAA
CACCCCATTATAAGGGGTACGAATAAAGTAACGGGGTTGTCGAAGCTCACCAGATTTTCACCAGGATG
CACGTCATACTCTTTTTTCTCCGGAGGCAGTGCCAGCATGGACTGCTGCTCTTCGAGCCAGCGATCACA
GGGACGGGCCTGGATTGTTTCATGCTTTCGTTGGTTAGCGACATCGTGCAGCCAGCGCAGACCGTGGC
GGTTGGCTGTTTCAACATCGACAGTGCTCCCCATCGGGCGCAGGCGAGTCATTAGTGGGATGTAAAAA
CTGTTACGGGTGTACTGCACCATCCGTTCCACCTTACCTTTAGTCTGTGCCCTGAAGGGGCGACACAGT
CGGGGAGAGAAGCCCATCTCCTTGCCGAACTGCCACAGCGAAGGATGGAACCGGTGCTGACCGGTCT
GATATGCGTCACGTTGCAGAACCACAGTTTTCATATTGTCATACAACACTTCGCGCGGCACACCACCAA
AGAAGCGGAACGCATTACGATGGCAGGTCTCCAGCGTGTCATAACGCATATTGTCAGTGAATTCGATG
TACAACATTCGGCTGTATCCGGGAACAGCAACGAACACGTGAAGCGGTGAGCGACCATTACGCATAGT
GCCCCAGTCAACCTGCATCTGTCGTCCGGGTTCAGTTTCGAACCGAACGGCAGGCTCCTGCTCCTGAGG
AACCGAGAGAGAACGAATGAATGCCCTGAGAATGGTCATTCCGCCACGATATCCCTGGTCTCTGATCTC
GCGAGCGATTACCGTTGCCGGGATTTTGTAAGGATGAGCATCGGCGATGCGTTGACGAATATAATCCC
GGTATTCATCCAGGAGTGAAGCAACAGCAGGTCGCGGCGTATATTTTGGCGGCTCAGATTTTGCCTGC
AAATAACGTTTAACGGTATTGCGGGAGAGCCCCAGTTCTCTGGCAATCGCCCGGCTACTCATTCCCTGC
TTGTGCAGGATTTTAATTTCCATAACTGTCTCAAAAGTGACCATAAGCTCTCCTGAATCAGGAGAGCAG
ATTACCCCCTGGATCTGATTTCAGGCGTTGGGTGTGGATCACTATTGCACCGTTCGTGACAGTGTTGTT
GCGCCTTGCTGCCAACGAAGCTCCACTGCCCATCAAGTTCACAGATAAGGGCAACATCAGCGTGAGCG
ACTGGCGCAGATGTTATCCGCCGGGGGGGGAGCTTTTTAATGTCCGTATGACGGTATTGATGCCGATTT
TCAGCGTACGGGCAGTATCGCGAACTCCGGCACCGTTGAAAGCCATTTCAGTGATTTGGTTTTTAACTC
CCGGTTTACGGGCCTCGTAACAGTAAGTGAGCTGAAACACCCGGTGACAGTCACGGCAACGGAATCGA
TCATGTCCTTTTGGGTTCTGCCCGTGACGGTAAACCTGAACGGACTGGCAACGGGGGCAATGAAGGTT
AACTGTGACCATGAAAGAACCTTAAAAGCGGGTATTATAACCCCTAATTCAACTAATTGGAGGCATTGT
CATTTTTAATCTATGGATACACTTAAAAATCCTCTCACCTTACGTTAAAAACCTTCTCATGTTTAGAGTTTT
TTATTATGTTTATCATAATGATATGACGATCTGAGTTTTTAAAATCAAGAAGAAAAGATAAGTTTTTTAA
ATGCACAGTGCCTGTGGATAACTTTGCGTAAATCCGGAGGACAGATCACTCTGAAGCAAGACAACAGC
ATCTGAAAACAATATGCTCACGTTTCAACCACAGGGCGTAAGTGATCCTACCCACGTAATATGGACACA
GGCCTAAGCGAGGTTCTGGTTTTCAAATTGTTCCGGACTGAGGCCGCCACACCAACTGTGCCGTCGCCA
GCGATTGTAATCACATTCGATATAATTAAACACTGTTGCCCGCATTATTTCCCGGCTGATAAAGTGTTCT
CCGTGGAGACATTCCACTTTCAGCGAATGAAAGAAGCTTTCCACGCAGGCATTATCGTAGCAGCAGCCC
TTTGCGCTCATACTGCCACGCAGATTATGCCACTTCAGCAGCGCCTGATAATCTGCTGAACAGTACTGAC
TTCCTCGGTCCGAGTGAACGATAACGTTCCGGGGTCTCTTACGCCGCCACAGCGCCATCTGCAGGGCAT
CGCAGGCCAGTTGCGCCGTCAGGCGTGGTGACATGGACCAGCCAATAACGGCACGTGACCACAGGTC
AATGACCACAGCCAGATACAGCCAGCCTTCATCTGTACGTAAGTACGTGATGTCTCCCGCCCACTTCTG
GTTCGGACCACTGGCGTAAAAATCCTGCTCCAGCAGATTTTCTGACACAGGCAGGCCATGTGCGCGGT
AACTGACCGGGCTGAACTTCCGGGAGGCCTTTGCCCTCAGCCCCTGACGGCGCAGGCTTGCCGCCACG
GTTTTTACGTTAAAGTGGTAACCCTGAGCACGCAGTTCATCCGTCAGGCGTGGTGCACCGTAACGCTGT
TTTGACCGGGTAAAGGCCGCCGCGAGGACAACGCTGTCGCAGTGCTGACGGAACTGCTGACGAGGGC
TTATCCCTGTCCGCCGCTGACACCACGTATACCAGCCGCTGCGGGCCACCCGGAGCACGCGGCACATTG
CCTTGATGCTGAACTCAGCCTGATGTTTTTCAATAAAGACATACTTCATTTCAGGCGCTTCGCAAAGTAT
GTCGCGGCCTTTTGGAGGATAGCCAGTTCCTCTGCCTGCTCCGCCAGTTGCCGCTTCAGGCGGGCATTT
TCAGCGGCCAGTTCACTTTCACGCTCAGACGAGGTCATCTGTTGCTGCAGTTTGCTGCGCCACGCATAG
AGCTGAGATTCATACAGGCTGAGTTCACGGGCTGCGGCAGCAACACCGATGCGTTCAGCAAGCTTCAG
GGCTTCACTGCGAAATTCAGGCGAATGCTGTTTACGTGTTTTTTTGCTGGTTGATACTGTTTTTGTCATG
TGAGTCACCTCTGACTGAGAGTTTACTCACTTAGCCGCGTGTCCACTATTGCTGGGTAAGATCAATTTGC
TCACTGGTATTTACTACGTGCATCGAATGAGTGATCAAGAGCCGTATGACGCGAGAGTGTCACGTACG
GTTCCGTGAGCAGCTGGAGAGTGAAACTCTCTCCGGCTGACTCGACTCGCAGGTTCCGACAGCGGTGA
CGAACATGCAGCGGTGTTGTTCTCGCTGATCGGCACACGCCGTCTGAACAATGTGGAGCCAGAAAAAT
GGCTGCGTTACGTTATTGAACATATCCAGGACTGACCGGCAAACCGGGTACGCGATCTGTTGCCTTGGA
AAGTTGATCTGACTTCTCAGTAAATATCAATATGATTCTGACGAGCCGCTTACCTTTGAGCAGAGAAAG
GGGAGAGGGATCCCACATCCATGAGCTAGGAAAATACAAAAACTGGCTATACAACAATTCAATATATT
AGGACTTACTTGCGAGGAGAAATATAGCACCGCTCAGGCAATTCCTGGGTATTTACTTTTCTTAGTTTCT
TTACTCGGGATGTTCTGTCATCAGAAAGGCGATTCTAAATTTGTAATATAGTCACTCATATTCCCACGTA
ATTTCTTTAGATCAATATATTGCACATTTTTCTCAAAAAACTGATCGGAAATAAGATTTGCAACATTGCTT
GTTGTATCATTTTTAACGCTTCTATTCATCCCTTCACAAAGTTCGTCCATTTTCTTTTTGAAATAACTGCTT
CCTGATACAAGATGATTTATGGAACTTTGTGCAGCATTGATGTCTAGAGAAATGCGATAATCTTTCTCAA
CCTGTTTAAATATCTGTTTACGGAGGTTATCGCCGATTTCTCGGCTGTGTTCTGCACAAAACTTATCAAT
ACATTGGTTTACCACCCGGGATACGTAGACTTTTTTTTCATCATTTTTCCAATCTGTAATATTTTCACCGA
TTTGTTCTGAAACAGAGCTCAAAATACTAGTATTTATTTTGGTTGATGATATTTTTACTATCCTTTTCCCC
ATTGTACAGCGGATAATCTCATCCACCTTTCCTGAAAAATATCTTTTGTGAGATAAACTGATTCCAAAAT
TATTAAAAGATGTTCCAAGCATTATAATCACTTTGTCATGTAAAAATAGTAACATTGTCATTATTTTAGCT
GATTCAAACAATTCTGTAAATGCTTTGAGTTCTTTACTCCCTTGGCTCAATGATGGACCGTCTGGAATCC
CAGAGACTAAACTCTCTGAGAAAGAGAGTATGAAAAAACTTGGGGCAGTCCCTAAATGAAGGGACCA
ACAGCGGTAAAATAACCGGCATCATTCATAATGATGCCCTGTTAGTTGTAGTTATGAAGATTTACGTCA
TTAAAATTGCTGTTCGTAGTGTCAGTCCTATGGTCTGGCATCAGCTGAGGATCGTAGCCGATACATCAC
TGGCTTCACGACATTCGTGTTGAGGCTATTCATGAAAACTCCACGCTGAAAACGCCGTTTTGTATAAAC
GGTCACGGTATGCCCGGAGCCACTGTTGCTGATGAATTCGATAAAACCTTGGCGTTTCTTGAAGCTATC
GTTAATGCGGATAATGAAACAACGATTGGTGAGATTCGCTCTTTTGCCGACGCCCTGGACGCTGTCCGG
TTCAATCGCAACAAAATCAACCGGCAGCTGAGCAAACCCAACCTTGCCTCTCTGGCGCTGGAGCATGAA
GTGATTTGGCTGGGACGTAGCCGCTGACAGCTTCTATAAAAGTATGATCCTACCCACGTAATATGAACA
CAGGCTTAAGTGAGGTTCTGGTTTTCAAATTGTTCAGGACTGAGACCTCCACACCAACTGTGCCGTCGT
CAGCGATTGTAATCGCACTTGATATAATTAAACACTGTATATTTCATTTGTGGAGTCTCCAGTCTGAGGG
GGCTTTATGCATTCCGTATTGGGCACTATGATGCCAGAAATCTGCGAGGCTCCACACCTTCCTGTATTTC
CCTTCCTCTATCACAGTGCACTTTACTTAATAGGGTGGGATGTGTTCATTACATTTATTGTGTTTTTTATG
ATTGTTTTTGCGTCCAGTGTCGTTCGTCATGGGAAACTGGTTCTATGGGTAGTATCGCTCAGTCTAGTTA
ACTGTTTGTTTCTTTTTAGAAATTAAGCAGATCACACAATTCGGGGAGAGATCCTTCTAAGTGCAATTCT
TTGCAGCTATTTAGCACACTTAAAACAACTCTTGTATTTTGTCAGAAATAATATTAAACATGTCTTGTTCA
TTATTGTTAAGTCTGTCTGCTATTTCTGTAAGTTTTATCCCACTATGATGGCATATATTTATATATGCCATC
ATAGCCTCGATATTCCCTTTCCCTGCGGCCAGAAATAATCCTGGTAAACCCTCATTATCTCTTACATCCAA
TAGTTTAATCGTTTCTTTTGGTGATAAATCAAAAGTTTTAATAAAATCTCCATAAGCTTGAATCGCAGCG
GAATGACCTTCTTGAAGAGCTATGAACAAAGCAGATTCCCCAGCATTATTTCGTGCTGCAAGAACTTCT
AGTTTTTCCTCTTTGGGTATTTCGGCTGCCAACAGCGATTCACCGTATTTACGCGCTGCATCGGCATTTC
CATGCCAAATCAATGAAAACAAATTATGGCGGTTATACTCCTTATTATTCTTTGTCGGAATGGATATGCT
CTGTATTTTGGCCAATTTTCTGAAAGAAACACCACTATATCCCCCCCCTGATTTTTCTTCATTGATGGCGT
GGGATATAGCGGAATTTGTCACGTTAATGATATTACTTTTCATTCCTTTATTTTCATTAGCAGTATTGCTG
TCTATTATTAGGTGCATATTTTTGTTGTTTGCTGGATGTAATCCATAGTTATTTATTGACATTTTTGTTCAT
CCTTTTTGCTAAAAAAAACAATAACCTATTTTTGTAATGTAATAGATATTAGTCTATTGCCAATCTAAAAA
AACCATTGCCACATTGAATGGCTATATTCACCAATAAATTCTTTCGATTTCGTATATTGACATATCGTAGA
TATATCTTATCCCTAAGATTCAATAATCGAGTCCGTTTACCATTCTGTGCAAGCGCCTTCACTCAGAAGT
GACCGTCAAGGCGATCACCAAACTTGATAATAAAGCAGCTCATTGCCATACGCCAGTCCTTAAGGGGC
GTTGTCCATTTCTGTGATGCTGCCTAGATTTAACTGCCAGCTCCGGCTTATCTACATCTAAATATGTGAG
CCTAATACCGCTACACTTTGGCCAGACTATGTTTTTCTCCGGGAAGTGGGCTAACCGTCTCCATAAAGTG
ACGAACTTTTTTCAACAGTGGCCATATTGAACGGCACTGATGATTACGGGTTATTGTCTCATGAAGAGC
CGGCCACATCACTGACAGCAGTGACTTCAGGCCTTCAGCATCAGACAGCCTGAACCAGTTAATCCATCG
CCCGACAGATTTAGTAATAAAGGGGCTTGGCAGTTTGGATAAACTATTGTTCTTATTCCACTAATACAAA
CTTTTTGTAAAGTATATTAAATATACTTCATTTCATACTGAAATCCCCGGAAGTATTCAATCAAACAATTA
CACCATAAAAGGAAGAGGGGGCCCTAATTAAGTTGTCAAGCATGTTTATGATACCTGCAGGGTATAAA
TTTTCTGGCTGCAAGCAGACACATATAAAACGTCTTTATTGATCCTACCCACATAATATGGACACAGTCG
TAAGTGAGGCTCTGGTTTTCAAATTGTTCCGGACTGAGACCACCACACCAACGGTGCCGTCGCCAGCGA
TTGTAATCGCACTCGATATAACTAAACACTGTTGCCCGCACTATTTCCCGATGGATAATAGTGTTCTCCG
TGGAGACATTCCATTTTCAGCGAATGAATTTTCATCTGTTGCTGTATCCAGTCATACAGTGACTGCATCA
GTGGCGCGGCTCTGGCTTTTCTTGCCGCCAGACGCTGTTCTGCTGAACATCCCCGGACTTCTGCCTCGAT
GGCATACAGTTCACCGATACGCTGCTGGGCTTCCGTGGTGATGTCGGTTGGCACTCTTGCATGCACATC
GTGGATTTTTCTCCGGACATGAGCCATACATGCGGCTTCCGTTATTCTGCCGGATTCGTACAACGCCCG
GTAACTACCGTAAGCATCGGCCTGAAGCACTCCGCTGTAACCGGCCAGATGATTCTGTGGATGGATACC
TTTCCGGTCCGGTGAGTACGCGAACCAGACCGCCGGAGGCATTTCCGAACCGGCGTTACGGTCATCAC
GAACGTAGACCCACAGCCGGGCAGTCCTTGTTTTACCGCTGCCCGGCTCCTGGACCGGAACGGGGATA
TCATCGGCATGGACTTTACCGGGCATCAGCACATACTGGCGCAGGATGTCATACAGCGGTTCCAGCAG
TTCAGCAACAGCACCTGTCCAGCGCCCCAGTGTGGCACGGCTCAGCTCCACGCCCTGACGACGGTATAT
TTCTGACTGGCGGTATAACGGCAGATAGTCTGCATATTTTCCAGCGACAACATGGGCCAGAAGCCCCGC
TCCGGCATAACTGCGTGCAATGGGTTTTGAAGGCACCGGTGCCTGCACGATATGGTCACACCGGCAAC
AGGCCAGTTTCGGACGTTGTGTTTCGATAACCTTAAAGGCGCTGCTGATAAGCTCCAGCTGCTCTGACA
CATCACATCCCAGAGAACTGAGTTCGCCACCACAGGCCGGGCAGCATTCCTCTTCCGGACGGATGACCC
GGGTTTCACGGGGAAGTGAGGCAGGTAACGGTTTATGGGCTGAAGACTGGCGCAGGGGAGATGGCA
GTACCGGGTCATATTGCTCACCCAGCGTTTCCGCCATTTCTTCCTGAAGTGCGCTGATTCGCTCCTGTGC
TTCCTGTATCTGCCGTTCGGTTTTTGCGCGAAGTTTTTCTGAGCTTTTACCGAACTGCATGCGCTGCAGTT
TCGCAACCAGCGCCTTCAGCCGGTTGATTTCGGAAGCATAAGCCGCCACCCGCTGTGAGAGCAGGCGG
TTGTATTTCGCCATCTGGCGGATGGTGTCCTGTTGCGTCTGCAACAGTGCCCGCAGGCGGGCATTCTCA
TGAGCAAGTGAGGTGTCCATATCCTCACTTTACAACGGGTTATATGCGGATGCCAGCGCGTTCCGTTCG
TTTCGGGTGTTTCCAGTCGATGCCCTCAAGAAGCATGGATAACTGGGCCGGAGTAAGGTGCACCTTGC
CGTCACGGGTGACCGGCCAGACGAAGCGGCCCCGCTCCAGGCGTCTGGTAAAGAGGCACAGTCCGTC
ACTGTCAGCCCACAACACTTTTATCTGGTCACCCCGGCGTCCGCGGAAGATGAACAGATGCCCGGAGA
ACGGGTCATCCTTCAGGACGTTCTGAACTTTTGAGGCCAGGCCGTTAAAGCCATTTCGCATATCGGTGA
TACCTGCAACCAGCCAGATACGCGAACCTGCAGGGAAAGATATCATCAGTGGCTGCTCCCTTTTATTTC
GCGGATAAGTATCTGTAATAACGCCGGAGTCAGTTTACCTTTAAGCCTGAGGAAAGGGAGCGCAATAT
GAAGAGCTCAGAAAACTGGGTAAAGGCGATCATCTGGTGAAGCTGAAAACCAGCCCGCAGGCACGAA
AAAAGTGGCCGGGACTGGGAAATGAAGTGACAGCCCGCCTGCTGACCGTGACGCGCAAAGGAAAAGT
CTGCCATCTGCTGACGTCGATGACGGACGCCATGCGCTTCCCCGGAGGAGAAATGGCGGATCTGTACA
GTCATCGCTGGGAAATCGAACTGGGATACAGGGAGATAAAACAGACGATGCAACTGAGCAGGCTGAC
GCTGAGAAGTAAAAAGCCGGAGCTTGTGGAGCAAGAGCTGTGGGGTGTCTTACTGGCTTATAATCTGG
TGAGATATCAGATGATTAAAATGGCGGAACATCTGAAAGGTTACTGGCCGAATCAACTGAGTTTCTCA
GAATCATGCGGAATGGTGATGAGAATGCTGATGACATTGCAGGGCGCTTCACCGGGACGTATACCGG
AGCTGATGCGCGATCTTGCAAGTATGGGACAACTTGTGAAATTACCGACGAGAAGGGAAAGGGCCTTC
CCGAGAGTGGTAAAGGAGAGGCCCTGGAAATACCCCACAGCCCCGAAAAAGAGCCAGTCAGTTGCTT
AACTGACTGGCATTAAGGCCGCCGGGGACGTTTTTTTACCACCGGGAGTTTGGGCATAACGGTGCTTTC
AGTAAGTACGACAGGTACTGTGGAGGCACTTCCGTAAAGAAGGGCATCAAGTTCCTGCTCCGACATAC
CTGCGGGCAGAGGCCATGAAAGGCCAGCGTTGCGGAAGCGCACGAACATACCACAGACAGTTGATTT
TGGTACGCCCAGGCGACGTCCGGCCACAACCCGTGGTAAATGTTCTTCGAAATGAAGGCGTAAAGCTT
CAGTGATCCAGGTCCGGTATTCCATACGATAGTGTCCACTTCAAAATGATGGACATTATTTTTGTGGAG
CCGGAGGAAACAGACCAGACGGTTTAAATGAGCCGGTTACGAAGAAGCTGAGGTTCTGCGCTTTGATA
ATTTTGACGATGCCGTTGAGTGTTGCGCAGACCTGAATATTCCGTTCTTTGTGGATGCCGGAAACAAAA
AACTGGTCTTCTGGTTTGTACGTGTTGATGACGAAGGGTATCCGGAAATTGCCCGCTGCATGGAGCGG
GAATTTGCGACAATTCCTGCCGGTATCAGCGCCGACGGCATGTATTGCCCTGAATGCGGTACGGTTCAC
TGGCCGGATGGCGTTATTCCGCCTTTCTGATGCTTCCCCGTTTTGCCGATATTTTTCAGCAGGGAAACCG
ATGGCTTAACTGGCTGGAGAAGCAGTCGGTGCAGATGAGCCGCCTGGAACATTATGCCGGTCAGGAT
GAAATTGGCCTGCGTTACAACAGCCACCGGACAAAACGAGAAGAAAACCTTGTGATGAGTGGCGACG
AGTTTATGGAAAGGTTCTCCTGGCATGTGGCGGATAAGGGGTTCCGGATGGTGATACGGGGTCCTGA
GTCCGGTGAAGCGGCGATTACTGGAAGATGTGGTGTACGCCATAACGGAGACAGTGAGAAAAACGGC
GAGGCAAATCACAAGGAGAGGGATGTATCAGCAGTGACTGAAGGTTGACCCGCTGAAGTGCGAGCTG
TGCGGAGGTCAGATGCGGTTTACGGGGCTGAAGAGGGGTTACCGTCTGGCAGAGCAGGTTATGATGC
ATGAGCCGCTGGCTCGAATACGGTGGTGCGGCTGAGAGTCGCAGAGGGGAAGTTGCGTCCATGTTGA
GGGGAACGGAGCAAAAAACCATCAGAGATACATTGAATCAATCAATGTAAGCAATATTAAGTGTCTAT
ACCGATTACTTGATGATCAGGTTGTGTTGAATAAACGCGCATTCTTCGGTGCTTTTTTAATCTTTTGGGG
ATTTAAATTCCTATCGATGAACTGTCGATATGAAAAAGCGAGCATCATACTGACGTCAAACAAAGGGGT
CGCAGACTGGGGAGAAATGTTCGGAGATCACGTGCTGGCAACAGCGATCCTAGATCGGCTGCTACACG
CTGAATATCAAAGGAGAGAGTTACCGGTTAAAAGAGAAACGTAAAGCTGGAGTGCTGACCAAAAACA
CAACGCCAATCAGTGATGATGAAATGGTGGAAAACGGACAGCATCAGTAACGAAAGTATCTTAGCAGG
CATGAAAATCGTCCGCATCCCCTAAACGGTCAAAATGCTTCTGCCGTGAAGGAGGGAGAATGCCATCTC
ATCACTTATATCTGGCGAATGAAACACAGATGGTCGGCCTTGCCCCCAAACACACGGCGGTGCAAAAT
GGAATGGCAGAAAACTTCGTCAAAACGATGAAATATGATTAAATCAGTGTTATGTTCGAACCTGACGT
GTTAACAGCAGTAAAGTAGTCTCGCGAAGGTGTTCAAACATTATAATGAATGGCATCCTCATAGTGCGC
AGGGTTATCGCTTGCTGCGGAAATATCTGAAGCGACGTACCAGTAATGGGGAAAATGATAGCTGGTTT
CTGAAAATATAGAGGAAAATGTGATATATATATCCTGTTAATATTTAAGGAGAATATAAAATAAAATGA
AAGTCTCGTTTAAGTCTTTAGGCTATATTTTTCATGATATATATAATAAAAAACACACTATCGATGAGTTC
AACGATGTGGTAAGAAAAGCAGTTTTATCAGGTAAAATTAACGAGCTGAATGCATGTCATAAAGTGGC
AATTTTTCTTGCCGAAAAAGACAATGAGATAACAAAGAAAGACAAAGCCAAGATAATAGATACATTAA
CTGAAAACTATTCCATTGAATTTCAACAATTAATGAATATTAGTGAGCGCACGTTAAATTCATCTTTATAT
ATTACTCCTGGTGAAAGTGGTTTTGTCAGTTTTGTTAATCGTGAAGGAAAAATATGTCATACTGCATATG
TAAAATCATCAGATAATAGCATGGCATATTACCATGCAAATTATAGCAGCATTGACAAATATATTACGG
ATATGTGCGGCCTTATTTGTATGAGGCATATTGAGTCTACTGGCATTATTTTTTATATGCTTGATGAAAA
AGTTTTATCTGCCATTGCTGAATTTATGAATGAAAAAGGATGGCGGGCTGCGTTTTGTTCAGCAAAGAA
CTTATATAAATGTGTTTAATTTTAGAAAAACTATTTGGTAAAATCCAACAAGAGTCGAGAGGTTCGCGTT
GCTTCAGCAGGGCCTCAGCCTGTTCAACCAGACTCAGTTGCAGGTCGTGACGCAGATTTAGTGGTGGT
GGTTGATTGTCAGATACCAGCCGATGATCTTGTAGTGCATATCATCAGACTTTGAGAAGCATATGGTTT
TGCGGGTAAGCCGCTTAAGATGTGTCCGCAGGTTCAGATTATGTCGCTCTATCCGCTGAGTGCATTTCT
TGCTTATAACATGACTGGTTGAGGACAATAAAGTTCGGTATACCGGCTCAGGCATCGGTCATGTAGAA
GGCAATGCTAAACTGGCTCAGCAAACTAAGAGCAGCGCGCATCCACTTACCCAATGCGGTTGAGAAAA
TAGCCTTCGATCGCTTTCATGTGGCGAAACAACCGGGTGAGGTGGTGGATAAAACCCGCCAGAATGAA
CATCCGCATCTTCCTGTTGAAAGTCGTCGTCAGGCAAAGGGAACTCGCTTCCTGTGGCAGCACAGCAAT
AAATGGATGACCGAATCCAGGCAGGAAAAGCTGATATGGCTGCGTGCGCAGATGAAGCTGACGAGCC
TGTGCTGGGCGCTGAAAGAACTGGCAAAGGATATCTGGAGCAGACCATGGAGCGAGGAAAGACGCAA
CGACTGGCAGAGATGGCTGTCGCTGGCGGCCAACAGTGACGTCCCCATGATGAAGAATGTGGCTAAA
ACGATAGGGAAAAGGTTGTACGGTATCCTGAACGCCATGCGCCATGGCGTATCAAACGGAAATGCGG
AAGCACTGAACAGCAAGATCAGGCTGCTGAGAATAAAAGCCAAGGGCTACCGCAACCGGGAGCGCTT
TAAGCTGGGAGTGATGTTCCACTATGGGAAACTGAATATGGCGTTCTAAGCCTTCCCACCATGATCGGA
GAAGACCCTTACGCTTACATACATTACATATAATGTATCGTTTTATAAAAGATGATAGACAATCAATATA
AGATAATATATCTATTTTATAGAGGACGTTTTCTATGCCCATAAAAAAGCCCTGTCTAAAACTAAATTTA
GATTCTTTGAATGTTGTGAGGTCTGAAATACCGCAGATGCTTTCTGCGAATGAAAGATTGAAAAATAAC
TTCAATATATTGTACAACCAAATACGGCAATACCCAGCGTACTATTTCAAAGTAGCATCGAACGTGCCA
AATTACTCTGATATATGTCAATTTTTTTCTGTCATGTACCAAGGATTCCAGATTGTGAACCATAGTGGAG
ATGTGTTTATTCATGCATGTCGTGAAAATCCTCAGAGTAAGGGGGATTTTGTTGGGGACAAGTTTCATA
TTAGTATAGCTAGGGAGCAAGTTCCTCTGGCATTCCAAATTTTGTCTGGTTTATTATTTTCAGAGGACAG
TCCTATAGATAAATGGAAGATAACTGATATGAATCGAGTTTCTCAGCAATCTCGTGTGGGGATAGGAG
CTCAGTTTACGCTATATGTAAAATCAGATCAGGAATGCTCGCAATATAGTGCTTTATTACTTCATAAAAT
ACGACAATTTATAATGTGTCTCGAGTCTAATCTATTAAGAAGCAAAATCGCTCCCGGGGAGTATCCGGC
GTCAGATGTTAGACCTGAAGACTGGAAATATGTCAGCTATCGTAATGAACTACGAAGCGATCGAGATG
GAAGTGAAAGGCAAGAGCAAATGTTACGAGAGGAACCATTTTATCGTTTGATGATAGAGTAGAGAAG
ATGCCAGCTACACCACCTTGTAGCTTGAAAACTGGTTAGAATAACTACGATTAGATTTGGCACTGCGTA
TGCAGCCTGATTCCTGCCCGTGACAGAGCATTCGGCTCATAAACTGCTGAGCTACTCATCTGAAATCCG
AACAGTTGTCCACCACTTCAGCGTGCATCTCCTGTTTGAGTCGTTTGATTGCTTTTCGGGATATTAACCT
GTCGGATTTTGCATTAGTTAATGCTCCCATCAGGCCTTCATCTTTTCCAGTGCCTATTTTCCTGTTCTCCTT
TTGCACAATATAATCAGATCATCTGTATCGTTGACGAGCTCGCTATCTTATAGGAGCATTCTGAATAAGC
TACCTCCTTGTAAGGAGGCTTTTACTATTAGTAACTGCAATCTAATAGTATTATAACATCTCTATGGTTGT
CTTCTATGCCGTTAAATAAAACATTTTCATCTAGTATATTTTCAACAAAAAATTCGTTATCAACAGATATG
TCTGTTAACAGAGATAATAGAACAATAACAAGTTCTATTATGAGGGTCTCAAACTCCAGTGAACTAATT
CAGTTTAAGAATAAAACAGCACCTTATTTTTCAGAAAAGAGAAATGTGAAGGTAAATATAAATGGAGT
GGCAAAAGATATATATGGAAGACAAATAGTTTGCCGTCACCTAGCTTCCTATTGGGAAATGAATTTTAT
GGAAACAAATGGGAAGGTAAACTATCAGCTTCTTAGCACCCCTGATGCTATTGCAAAAAATGTATGTTT
GGAAAAAACGGAAGATTTTTCAAAATCACCAGCTTATATTTATTTTGTGGAAAATAAAAAGTGGGGAAC
AGTTATTACTAATTTTTTTTATAACATGAAAAAAAATGGAGACTTTATCAGAACATTATCAGCATGCACA
TTAAACCACCAAATGGCGTTAGGGTTGAAAATAAAGCGCGTACAAGAAAGTGAAAAATGGGTTGTTCA
GTTTTTCGATCCTAATCGAACTGTTACTCATAAACGTACTGTATTTACTTGTGATAGCCATTTCGAACTGA
GTCAATTATCCGCTAAGGATTTTTTTGATGATTTTTATTGGAAAATTTATGGTCTGGAGCAACCCGGGCA
GGTAATATTTGAAGATAGGCATAATTCCCCTCTAACAAACACAGTGAAACTTTTACCAGATGAATTGAT
CAATTCCAGAGTTATATATCATGCGATAACAAAGAATCTGACAGAAGTATTATTTATACTGATGGAAAA
ATACAAAAATGGTGAAATTTCTCAATCTAAACTGGTGAACCTTCTGGCCACGAGAAGCTCTGATGGTAC
TCCTGCATTTTATATAGCACTTCAAAACGGATGTTCAGACATTATTCAAGTATATGGTAAGATATTAAAT
ATGTGCAATCTTAGTCAAGAAACAATACTTACATTGCTTGCTGCGGTAGGGGCAAACAATGTGCCGGGT
TTATGCATGTCTTTTATGAACGGACATGTAGACACAATAAAAGCCTATGGGGAAATAGTCTTTAAAACC
CCGTTAACTTCCGATAAGAGATTATATCTATTAGCTGCGAAAGACAGCCATGATTTACCTGGGTTATTTT
TTGCATTGCAGAATGGACATGCAGATAGTATTCGTATGTTCGGAAGTCTGCTTAATAAGAAAATGCTTA
GTAGTGAACAAATTAAAGAATTATTGAAAGTAAAGCATGGGTTGTTTATGGCACTTCAAAATGGGCAC
ACAAAGGCCATTATGGCGTATGGAGATATACTAAAAATACTCCCCCCTCACCAGGAATATATAGATGAA
TTATTATGGATAAAAAATCCCAACGGTACATCAGGACTTTTTATGGCATTTTATAATGGCCATACCGAAA
CAATACGTGCCTTTTGTAATATTCTAAAAAACTATAGTTTTACCACAAGACGATTGGTCGAAATGCTGTC
CGCAACTAATAAAGATGGTATTCCTGGTGTATTTGTATCTGTAGTAAATAGAGACAAAGAAACAATATT
AGAGTATTGTAGAATAATAAAAGAGAACAATCTGGAACCGGATACTATAGCAGAACAATTTTCAAAGA
AGATGAAAAAAACGTTTATTGAAATAATAAATAGATTTAATCATTTTTTATGAGACTGTAATTTAAATTG
TGTAATTACCTGTTTTTGATATGTTCACTCCAACAAGGTGTAGTGACCGGGTAACTGCCAACTTTCAGGC
AACCACCTCGATTAGCAACCTTATTTCCATATCGAGTTCAAATATCCCGTAGGGGTTTACATGGGCGTAA
ACTGATGATTGTTCAGGTTTCCATGGTGTGGCCAAATCTTCTTCATGGATAAAGGTCACATCCAGAGGG
TGAGTATGTTCCAATATTTAATACTAAAGATATGAGGCTTGGAATTGGGTTGCATCTTATTGATTTTATA
AGAAAGAGTAAAGATCAAGGATTCCGAGAGTTTTGTTATAACAAAAACATTGATCCTGTTAGTCTGGAT
AGAATTATAAACTTTGTGTTTCAGCTTGAGTATCATATACCAAGAATGCTAAGTACAGATAACTTCAAAA
AAAATAAAACTCAGAGACATATCTTTGGAGGATGGATTTGCCCCTATATTTCCAGACATCTGTTATCACT
TAACCCATTACAAGCCCGCTGCCGCAGATATTCCCGTGGCGAGCGATAACCCAGCGCACTATGCGGAT
GCCATTCGTTATAATGCTCGAACGCCTCTGCAAGGTTCTTTGCTGCCGTTAACCCGTCTGGTTTGGGCAT
GATACTGATGTAGTCACGCTTTATCGTTTTCACGAAGCTCTCTGCTATTCCGTTACTCTCCGGACTCCGCA
CCGCCGTGTTCTTCGGTTCAAGTCCCAACATCCGGGCGAACTGGCGTGTTTCATTAGCCCGGTAGCATG
AACCATTATCCGTCAGCCACTCCACTGGAGACGACGGAAGATCGTTGCCGAAGCGGCGTTCCACCGCTC
CCAGCATGACGTCCTGTACTGTTTCACTGTTGAAGCCGCCGGTAGTCACCGCCCAGTGCAGTGCCTCAC
GATCACAGCAGTCCAGCGCGAACGTGACACGCAGTCTCTCTCCGTTATCACAGCAGAACTCGAACCCGT
CAGAGCACCATCGCTGATTGCTTTCTTTCACGGCCACTCTGCCTGTATGTGCCCGTTTCGATGGCGGTAC
AGCAGGTTTTCGCTCAAGCAACAGCGCATTCTGGCGCATGATCCGGTAAACACGTTTGGCATTGATCGC
AGGCATACCATCAAGTTCTGCCTGTCTGCGAAGCAACGCCCATACCCGACGATAACCATACGTGGGCA
GCTCTCCGATAACATGGTGTATACGGAGAAGCACATCCGTATCATCAGTGTGACGACTGCGGCGGCCA
TCCATCCAGTCATCGGTTCGTCTGAGAATGACGTGCAACTGCGCACGCGACACCCGGAGACAACGGCT
GACTAAGCTTACTCCCCATCCCCGGGCAATAAGGGCGCGTGCGCTATCCACTTTTTTGCCCGTCCATATT
CAACGGCTTCTTTGAGGAGTTCATTTTCCATCGTTTTCTTGCCGAGCAGGCGCTGGAGTTCTTTAATCTG
CTTCATGGCGGCAGCAAGTTCAGAGGCAGGAACAACCTGTTCTCCGGCGGCCACAGCAGTAAGACTTC
CTTCCTGGTATTGCTTACGCCAGAGAAATAACTGGCTGGCTGCTACACCATGTTGCCGGGCAACGAGG
GAGACCGTCATCCCCGGTTCAAAGCTCTGCTGAACAATTGCGATCTTTTCCTGTGTGGTACGCCGTCTGC
GTTTCTCCGGCCCTAAGACATCAATCATCTGTACTCCAATGACTAGTCTAAAAACTAGTATTAAGACTAT
CACTTATTTAAGTGATACTGGTTGTCTGGAGATTCAGGGGGCCAGTCTAGAGGATGCGATTAAAGCATC
TAATTATGAAGAGATTAACAATAAAGTGACTGATAAAAAAATGGCTCATCAGGCCCTTGCTTATTCTTTA
GGCAATAAAAAAGCTGATATAGCACTTTATTTACTATCTAAATTTAATTTTACAAAACAAGATGTCGCAG
AGATGGAGAAAATGAACAACAATAGATATTGTAATCTGTATGATGTTGAATACTTACTTAGTAAAGATG
GTGCCAACTATAAAGTGCTAGAATATTTTATCAATAATGGATTGGTTGATGTAAACAAAAAATTTCAAA
AAGCAAATAGTGGGGACACTATGCTTGACAATGCAATGAAAAGCAAAGATTCAAAAATGATTGATTTTT
TATTAAAAAATGGAGCGGTATCAGGCAAACGATTTGAGAGGTGACCTAAAATATATTTAGGTTGTGCC
CTGAATCTGAAGTTGTCATTTCTGTTATATTGATCGACATAGGGGATGGTGCAGACCGTTCCCCTGCAA
CACCACATTGCATGTGGTGAACGGAACCGCCCCGAGTTTCCTAGAGAATGTTTTATCTGTGAACTCAGG
TTGCCAGATCATCGTTTCCGATGGCAGCATAATAAGCTTTTTCTGCTTCTGCCGGAGGGATATGTCCCAG
CCTTTCCAGTAATCCCTTAAATTCAGGGGTAAAACGTGGTGTGCCCATAGACTCCTCCTATGCTCATATA
GGGTAGATTTGTCTATGGGCCCTCCATAGCCTGATACAGGCTGTCCAGCTTTATGGGGTCAGTTCATTG
GTAGGAGTATTTAATTCTTCAAACTCTTTTTTGGCTTCGCTAAGGCTTTTGGTAAATTCAGGTGTATTATG
AAGTACTGCAACAACCGATGCTCCCATTAAACGCCCAGCCTCTACATCGCTTTGCCAATGCGCACCGCA
GATGACCCGACTTTCTCCAAACTCATATCCACGTCGAAGTATTTCCGCTTTACGTTGAGGATTAATCTCC
GCAAGTATCAGTGCTACTGCCCAACCAAAGGATGCATGACCAGAGGGATAAGAGCCAGTGATAGCCAT
TTTCTCATCTTTATCAGGTGTACAGGTTGCGTCTTTATAAATAACGAATGGACGAACTCTTTTATAATATT
CTTTGGCGTTACGCACGGCGTAATCATGGCTATCCTGTAGTACTTGTGTTAACAACTGATATAAGATTG
GGGTATTTTCCCTTGAAATATCCATGCCATAAGCATCTGAAAATGCAAGACCAAAATTCTCGTAGTATGC
ATCTTCGCTAGCGAGACGTACACGATTAGCATCCCGGAGCGAGCGGCCGAATTCATAATGAGCTTTGTC
AGCCTGAAATACTACTGAATCCTCTGCCGGAGGCGGCGGAAGTATTGACAAACTGTCTGGTGAAGTTT
GTTGAGTGAGAAAACCTTCTGCCTTCAGAGCATTTGCTGAGGGGATAAAAATCATATTTGTAGCAATAC
AAAAAAGAAGAAAGTTTTTGGTTTTCATAATTTATTTCCTTAGCATAATTAATTGATGATTTTGGTCAGCT
GTAAAAAACGAGCTGTGTTACAAAAAACACAAGTCATGCGGAAAAATTTGAACAGAGTGATGGAAAA
ACAAAAAATAGTCTTATATTAAAGTAATAACATTATTTTTCATATTCACGAAAAATAATGTGGTAAAAGA
ATGATGTTTTTTATCTATTTGTTCGTATCGTTTTGTCTCTGTATTATGATGATTTGTGATTTTCCCTCATAG
GGAAGCACTTTTCTACTAAAGATAAAATAACTTCATCTTAAATGCAAACAATTAACTATAGGTTAAAAAT
TTTATACTAATCCAGTTCTTTATTAATAATAAAAGTCTTTTTCTGAGAGCTTCTTTTTACTGTATGCTCCGG
ATCAGCAGGAATTGAAGTTGAACGATAATGTGAGTAGGGAAAAAGCTCTTGACCATAGTGAACTCCAT
ATCCATGATAGCCGGATATTTTTAGACCATCACTCAATGACTCATCGTTTTCAAGGTGTTTTTTTATCTGC
TCTAGCAAAGATTCTTTTTCCTTAAAAAAAGGCAGAGAGTTAAGGATACAAGAAAGACTTTCAGCAGG
GGCATTGTTAAAATTTTTAGGAGCCACTTTATCAGCGGAGCCGCATGAAGTAAAACGGATATCTTTCAC
ATTTAGAATACCACAGTCTTTCATTCTTGAAATGATTGTTTGTACATCAATAAGTTCCGAACCAAGGCCT
AATTGATGAGAACCAGGACTACCGTGCCCAAGAATATAAACCTTATTATACTCTTTAAAATCAGTATCGT
TTACTGTTTTTATTTTCGCTTTTTTTACAGAAAAATAAGCAGCAGCATTCAATGCGTCATCATCATTATTT
ATTACTATTGGTTCACTCTGTGGTAGCCATGATTCAGCCATTTTTAGAAAATCATTTTTACTTTTTTCATTT
TTAAAAACATAATCATATAACATCTTGTTCTTAAATACTCTTGGTATTTGTTGTCCCATAATAGATAGAAC
ATCATTGCTATCACAAAAACCTTTCTCTCCTAAAAAAACCAATGCTGCTTTTGGGTGAGTACATGATGAA
TTTTTTACCCTTTTTATTATATGTGCAAATGCAATATCTGATATGCTTTCATTCTTTTTATTGACTATTCTAC
AGTATGGTCTAACACCATCTAAATTCATACCTACATGACTCCATATATTGTTTTATAAACATCATAGTAAA
GGGTGTTATATTTTCATATAGTATGCAAGCTTCTTTGTATACTATGATTTAAAAAAACGGAACAAAAATG
CAATTATATTCTGGCATTGTTTAATGCTGTTTGTCTTTTTTATTCATTATTTCAATGAAAGAATTTCAACTA
CAAAGTTAATTGATCCTACCTACATAATATAGACACAGTACCTAAAGTACCGTGGAGATCATCCGGGGA
AGCCCCGGGTCTGATACCAGTGCAGGTAGACCGCTATCTTGTTCCGGTAATGTTCTGCCGTTCTTTACG
GCATCACCTCTAGCAGAAACATCGCGTAACTGCGCCAAGTATGATGAACTTCTCTTACCGCTGCGATCT
GGTTCAACAATATGCAGATGGTACACAACCTCTTTAACCAGCCCATCCGGATTATTCAGAGCCGCTTCC
GCAATCCGGAATAAAAGTGCTGTTTTACCGTGAACCCTCTGAAAATCAGCGAGCAGTGTTTTAACCAGC
CGACGTTCAGAGCGTACTGACAGACGGTGGACAATTTGAATAAACAGTTCAATCAGACCGTAGATGCT
CTCCCGTTGGCGACATGCTGGGAGCGGTGGAACACCGCTTCGGCAACGAGCTTCCGGCGTCTCCAGTG
AAGTGACTGACGGATAATGGTTCATGCTACCGGGCTAATGAAACACGCCAGTTCACCCGGATGTTGGG
ACTTTGTTGAACCGAAGAACACGGCGGTGCGGAGTCCGGAGAGTAACAGAATAGCAAAGAGCTTCGT
GAAAACAATAAAGTGTGACTACATCAGTATCATGCCCAAACCTGACGGGTTAACGGCAGCAAAGAACC
TTGCAGAGGCATTCGAGCATTATAACGAATGGCATCCGCATAGTGCGCTGGATTATCGCTCGCCACGG
GAATATCTGCGGCAGCGGGCCAATGATAACAGGTGTCTGGAAATATAGGGGCAAAGCCACCCACGAA
CGATCCAGCATATAATCAAGAAATTAGATTTCATTTAACATAAGATAGATTATGTGCACCAAGAGGCGT
TTCCTTAATTTCTGAATTTACAGGCTCTTTTGCTCTCTTTGCTATGCTGTGGATTTGGAAAGATAGAGTGC
TGGCTGATTATTTAGGTATTTTCATCAGCAT

1. An isolated plasmid encoding a bacterial VapBC toxin-antitoxin system, wherein the VapBC toxin-antitoxin system comprises a modified VapB amino acid sequence arranged to increase the maintenance of the plasmid in a bacterial host, wherein the modification to VapB comprises or consists of a substitution of at least one of Q12, F6, F51 or F60 residues relative to Shigella sonnei wild-type VapB or Shigella flexneri wild-type VapB, or structurally equivalent residues of Shigella sonnei wild-type VapB or Shigella flexneri wild-type VapB in a homologue thereof.

2. The isolated plasmid according to claim 1, wherein the modification to VapB comprises or consists of a mutation corresponding to Q12 of SEQ ID NO: 1 or SEQ ID NO: 2.

3. The isolated plasmid according to claim 1, wherein the Q12 mutation comprises or consists of a Q12L or Q12A mutation.

4. The isolated plasmid according to claim 1, wherein the modification to VapB comprises or consists of one or more phenylalanine substitutions.

5. The isolated plasmid according to claim 4, wherein the modification to VapB comprises or consists of a substitution at F6, and wherein the phenylalanine substitutions comprise or consist of one or more of F6, F51, or F60 of SEQ ID NO: 1 or SEQ ID NO: 2, or a structurally equivalent residue of Shigella sonnei wild-type VapB or Shigella flexneri wild-type VapB in a homologue thereof.

6. The isolated plasmid according to claim 1, wherein the modification to VapB comprises or consists of a substitution at F6, and wherein the F6 mutation comprises F6A, F6S or F6G.

7. The isolated plasmid according to claim 1, wherein the modification to VapB comprises or consists of a substitution at F51, and wherein the F51 mutation comprises F51A, F51S or F51G.

8. The isolated plasmid according to claim 1, wherein the modification to VapB comprises or consists of a substitution at F60, and wherein the F60 mutation comprises F60A, F60S or F60G.

9. The isolated plasmid according to claim 1, wherein the modified VapB amino acid sequence comprises or consists of the sequence of any of SEQ ID NO: 3-13.

10. The isolated plasmid according to claim 1, wherein the plasmid comprises pINV or pSTAB.

11. The isolated plasmid according to claim 1, wherein the plasmid is any plasmid of E. coli, Shigella spp., Salmonella spp., Enterobacter spp., Pseudomonas spp. or Yersinia spp., or other pathogenic bacterial species.

12. The isolated plasmid according to claim 1, wherein the plasmid is an expression plasmid encoding one or more polypeptides or nucleotides for expression.

13. The isolated plasmid according to claim 12, wherein the polypeptide or nucleotide for expression is a therapeutic molecule, a drug peptide, a pro-drug, a toxin, or an antigen for a vaccine.

14. A bacterial host cell containing a plasmid according to claim 1.

15. The isolated plasmid according to claim 1 or a bacterial host cell containing a plasmid according to claim 1, wherein the bacterial host is a Gram-negative bacterium.

16. The isolated plasmid according to claim 1, or a bacterial host cell containing a plasmid according to claim 1, or a bacterial host cell containing a plasmid according to claim 1 wherein the bacterial host is a Gram-negative bacterium, wherein the bacterial host is not S. sonnei CS14.

17. A nucleic acid encoding a modified VapB amino acid sequence arranged to increase the maintenance of a plasmid or genetic element in a bacterial host, wherein the modification to VapB comprises or consists of a substitution of at least one of Q12, F6, F51 and F60 residues relative to Shigella sonnei wild-type VapB or Shigella flexneri wild-type VapB, or structurally equivalent residues of Shigella sonnei wild-type VapB or Shigella flexneri wild-type VapB in a homologue thereof.

18. The nucleic acid according to claim 17, wherein the VapB is encoded in a VapBC toxin-antitoxin system in the nucleic acid.

19. A modified VapB polypeptide, the modification comprising or consisting of a substitution of at least one of Q12, F6, F51 or F60 residues relative to Shigella sonnei wild-type VapB, or structurally equivalent residues of Shigella sonnei wild-type VapB in a homologue thereof.

20. The modified VapB according to claim 19, wherein modified VapB polypeptide is isolated from a cell or is in a host cell, wherein the modified VapB is heterologous to the host cell.

21. A bacterial host cell comprising the modified VapB according to claim 19 or comprising a wild-type VapB of S. sonnei CS14, wherein the VapB of S. sonnei CS14 is heterologous to the bacterial host cell.

22. The bacterial host cell according to claim 21, wherein the bacterial host cell is not S. sonnei CS14.

23-24. (canceled)

25. A method of producing a polypeptide or nucleic acid product, the method comprising the cultivation of a bacterial host according to claim 21 wherein the bacterial host encodes and expresses the polypeptide or nucleic acid product; and optionally purifying/harvesting the polypeptide or nucleic acid product.

26. A method of antibiotic screening, the method comprising exposing a modified bacterial host according to claim 21 to one or more agents, and determining if the agents inhibit or kill the bacterial host.

27. A method of modifying a VapBC toxin-antitoxin system encoded in a nucleic acid, the method comprising modifying the nuclei acid to encode a modified VapB according to claim 19.

28. A method of enhancing the stabilisation/maintenance of a plasmid in a bacterial host, the method comprising the insertion of the nucleic acid according to claim 17 into the plasmid; or
wherein the plasmid encodes a VapBC toxin-antitoxin system, the method comprising modifying the nuclei acid to encode a modified VapB the modification to VapB comprising or consisting of a substitution of at least one of Q12, F6, F51 or F60 residues relative to Shigella sonnei wild-type VapB, or structurally equivalent residues of Shigella sonnei wild-type VapB in a homologue thereof.
